(19) United States
(12) Patent Application Publication
(10) Pub. No.: US 2025/0346602 A1
(43) Pub. Date: Nov. 13, 2025

(54) LACTAM SUBSTITUTED IMIDAZOPYRIDAZINE IL-17A MODULATORS AND USES THEREOF

(71) Applicant: DICE ALPHA, INC., Indianapolis, IN (US)

(72) Inventors: Hassane BELABED, South San Francisco, CA (US); Gary Edward Lee BRANDT, Alameda, CA (US); Paul R. FATHEREE, San Francisco, CA (US); Michael David FREIDBERG, Belmont, CA (US); Maureen Kay REILLY, Emerald Hills, CA (US)

(21) Appl. No.: 18/867,276

(22) PCT Filed: May 19, 2023
(86) PCT No.: PCT/US2023/067259

§ 371 Date: Nov. 19, 2024

Related U.S. Application Data
(60) Provisional application No. 63/343,756, filed on May 19, 2022.
(60) Provisional application No. 63/343,769, filed on May 19, 2022.

(51) Int. Cl. C07D 487/04; A61K 31/5025; C07D 519/00

(52) U.S. Cl. CPC C07D 487/04; A61K 31/5025; C07D 519/00

(57) ABSTRACT

The disclosure herein provides lactam substituted imidazopyridazine compounds of Formula (I), (II), (II-a), (II-a), (III), (IV), (IV-a), and (IV-b), or pharmaceutical compositions thereof, for the modulation of IL-17A. These compounds are useful in the treatment of inflammatory conditions such as psoriasis.

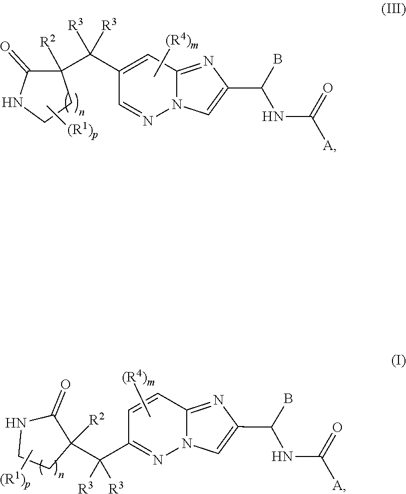

### CROSS-REFERENCE TO RELATED APPLICATIONS

This application claims the benefit of U.S. Provisional Patent Application No. 63/343,756, filed May 19, 2022, and U. S. Provisional Patent Application No. 63/343,769, filed May 19, 2022, each of which is incorporated herein by reference in their entirety.

### BACKGROUND OF THE INVENTION

The IL-17 family consists of six cytokines (IL-17A through IL-17F). Interleukin-17A (IL-17A), is an established pro-inflammatory cytokine, which is involved in the induction of IL-6, IL-8, G-CSF, TNF-α, IL-1β, PGE2, and IFN-γ, as well as numerous chemokines and other effectors. IL-17A can form homodimers or heterodimers with its family member, IL-17F and can bind to both IL-17 receptors, IL-17 RA and IL-17 RC, in order to mediate signaling. IL-17A is a major pathological cytokine expressed by Th17 cells, which are involved in the pathology of inflammation and autoimmunity, and also CD8+ T cells, γδ cells, NK cells, NKT cells, macrophages and dendritic cells. Additionally, IL-17A and Th17 are necessary for defense against various microbes despite their involvement in inflammation and autoimmune disorders. Further, IL-17A can actin cooperation with other inflammatory cytokines such as TNF-α, IFN-γ, and IL-1β to mediate pro-inflammatory effects.

To date, there area few biologics (Secukinumab and Ixekizumab) that have been approved to modulate IL-17A for the treatment of inflammatory diseases, such as psoriasis, ankylosing spondylitis, and psoriatic arthritis. These treatments require injection to a patient as they are not readily absorbed by the gut when orally ingested. Further, these approved biologic treatments have a high cost of entry for patients, limiting the availability to the patient population in need thereof.

There are a few small molecule modulators of IL-17A that have been approved for oral administration. However, while these have the convenience of oral administration and a lower cost of entry for patients, they lack the efficacy of approved biologics. Therefore, there exists a need for the development of potent small molecule IL-17A modulators for the treatment of inflammatory diseases and other associated disorders.

### SUMMARY OF THE INVENTION

In certain aspects, the present disclosure provides a compound represented by the structure of Formula (I):

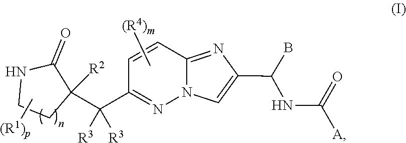

or a pharmaceutically acceptable salt thereof wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹,
   —N(R¹¹)S(O)₂R¹¹, —C(O)OR¹¹, —OC(O)R¹¹, —S(O)R¹¹, —S(O)₂R¹¹, —NO₂, —CN;
   C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂,
   —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹,
   —C(O)N(R¹¹)₂, —N(R¹¹)C(O)R¹¹, —C(O)OR¹¹, —OC(O)R¹¹, —NO₂, ═O, ═N(R¹¹), and —CN; and
   C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN;
B is selected from —C(H)(R⁵)₂ and C₃₋₁₀ carbocycle, wherein each C₃₋₁₀ carbocycle is optionally substituted with one or more substituents independently selected from:
   halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
   C₁₋₁₀ alkyl and C₃₋₁₀ carbocycle, any of which is optionally substituted with one or more substituents independently selected from halogen, —OR¹², —SR¹², —N(R¹²)₂,
   —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹²,
   —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁵ is independently selected at each occurrence from (i), (ii), and (iii):
   (i) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³,
      —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (iii) C₃₋₁₀ carbocycle optionally substituted with one or more substituents independently selected from:
      halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂,
      —N(R¹³)C(O)R¹³, —C(O)OR¹³, —OC(O)R¹³, —S(O)R¹³, —S(O)₂R¹³, —NO₂, ═O, ═S, ═N(R¹³), and —CN;
each R¹ is independently selected at each occurrence from:
   halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN;
   C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN; and
   C₃₋₁₀ carbocycle optionally substituted with one or more substituents independently selected from halogen, —OR^{14A}, —SR^{14A}, —N(R^{14A})₂, —C(O)R^{14A}, —C(O)N(R^{14A})₂, —N(R^{14A})C(O)R^{14A}, —C(O)OR^{14A}, —OC(O)R^{14A}, —S(O)R^{14A}, —S(O)₂R^{14A}, —NO₂, ═O, ═S, ═N(R^{14A}), —CN; and C₁₋₁₀ alkyl optionally substituted with one or more substituents independently selected from halogen, —OR^{14A}, —SR^{14A}, —N(R^{14A})₂, —C(O)R^{14A}, —C(O)N(R^{14A})₂, —N(R^{14A})C(O)R^{14A}, —C(O)OR^{14A}, —OC(O)R^{14A}, —S(O)R^{14A}, —S(O)₂R^{14A}, —NO₂, ═O, ═S, ═N(R^{14A}), and —CN; or
two R¹ substituents may come together to form a C₃₋₆ carbocycle, wherein the C₃₋₆ carbocycle is optionally substituted with one or more substituents independently selected from:
   halogen, —OR^{18A}, —N(R^{18A})₂, —C(O)R^{18A}, —C(O)N(R^{18A})₂, —NO₂, ═O, ═S, ═N(R^{18A}), —CN; and C₁₋₁₀ alkyl optionally substituted with one or more substituents independently selected from halogen, —OR^{18A}, —N(R^{18A})₂, —C(O)R^{18A}, —C(O)N(R^{18A})₂, —NO₂, ═O, ═S, ═N(R^{18A}), and —CN;
R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN; or
each R⁴ is independently selected at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R^{17A})C(O)R^{17A}, —N(R¹⁷)S(O)₂R¹⁷, —C(O)OR¹⁷, —OC(O)R¹⁷, —S(O)R¹⁷, —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, and —CN;
R¹¹, R^{11A}, R¹², R¹³, R¹⁴, R^{14A}, R¹⁵, R¹⁶, R¹⁷, R^{17A}, and R^{18A}, are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2;
m is selected from 0, 1 and 2; and
p is selected from 0, 1, 2, 3, 4, 5, and 6.

In certain aspects, the present disclosure provides a compound represented by the structure of Formula (III):

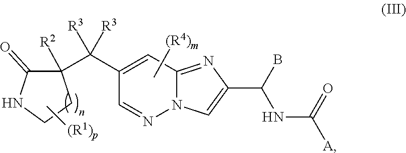

or a pharmaceutically acceptable salt thereof wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
   C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂
   —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
   C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂—N(R^{11A})C(O)R^{11A}, —C(O)OR^{11A}, —OC(O)R^{11A}, —NO₂, ═O, ═N(R^{11A}), and —CN;
B is selected from —C(H)(R⁵)₂ and C₃₋₁₀ carbocycle, wherein each C₃₋₁₀ carbocycle is optionally substituted with one or more substituents independently selected from:
   halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
   C₁₋₁₀ alkyl and C₃₋₁₀ carbocycle, any of which is optionally substituted with one or more substituents independently selected from halogen, —OR¹², —SR¹², —N(R¹²)₂,
   —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹²,
   —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁵ is independently selected at each occurrence from (i), (ii), and (iii):
   (i) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³,
      —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (iii) C₃₋₁₀ carbocycle optionally substituted with one or more substituents independently selected from:
      halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂,
      —N(R¹³)C(O)R¹³, —C(O)OR¹³, —OC(O)R¹³, —S(O)R¹³, —S(O)₂R¹³, —NO₂, ═O, ═S, ═N(R¹³), and —CN;
each R¹ is independently selected at each occurrence from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴,
   —S(O)₂R¹⁴, —NO₂, ═O, ═S, ═N(R¹⁴), —CN; and C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN;
R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN; or
each R⁴ is independently selected at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷,
   —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN;
R¹¹, R^{11A}, R¹², R¹³, R¹⁴, R¹⁵, R¹⁶, and R¹⁷ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2;
m is selected from 0, 1 and 2; and
p is selected from 0, 1, 2, 3, 4, 5, and 6.

In certain aspects, the present disclosure provides a pharmaceutical composition comprising a pharmaceutically acceptable excipient and a compound or salt of Formula (I), (II), (II-a), (II-b), (III), (IV), (IV-a), or (IV-b).

In certain aspects, the present disclosure provides a method of modulating IL-17A in a subject in need thereof, comprising administering to the subject a compound or salt of Formula (I), (II), (II-a), (II-b), (III), (IV), (IV-a), or (IV-b), or a pharmaceutical composition thereof.

In certain aspects, the present disclosure provides a method of treating an inflammatory disease or condition comprising administering to the subject a compound or salt of Formula (I), (II), (II-a), (II-b), (III), (IV), (IV-a), or (IV-b), or a pharmaceutical composition thereof. In some embodiments, the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, palmoplantar psoriasis, spondyloarthritis, and Non-infectious Uveitis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

“Alkyl” refers to a straight or branched hydrocarbon chain monovalent radical consisting solely of carbon and hydrogen atoms, containing no unsaturation, and preferably having from one to twelve carbon atoms (i.e., C₁-C₁₂ alkyl). The alkyl is attached to the remainder of the molecule through a single bond. In certain embodiments, an alkyl comprises one to twelve carbon atoms (i.e., C₁-C₁₂ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). For example, the alkyl group may be attached to the rest of the molecule by a single bind, such as, methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl), and the like.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. Alkylene chain may be optionally substituted by one or more substituents such as those substituents described herein. In certain embodiments, an alkylene comprises one to ten carbon atoms (i.e., C₁-C₁₀ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. Alkenylene chain may be optionally substituted by one or more substituents such as those substituents described herein. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. Alkynylene chain may be optionally substituted by one or more substituents such as those substituents described herein. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

The term “C_{x-y}” when used in conjunction with a chemical moiety, such as alkyl, alkenyl, or alkynyl is meant to include groups that contain from x to y carbons in the chain. For example, the term “C₁₋₆ alkyl” refers to substituted or unsubstituted saturated hydrocarbon groups, including straight-chain alkyl and branched-chain alkyl groups that contain from 1 to 6 carbons. The term —C_{x-y} alkylene- refers to a substituted or unsubstituted alkylene chain with from x to y carbons in the alkylene chain. For example, —C₁₋₆ alkylene- may be selected from methylene, ethylene, propylene, butylene, pentylene, and hexylene, any one of which is optionally substituted.

The terms “C_{x-y} alkenyl” and “C_{x-y} alkynyl” refer to unsaturated aliphatic groups analogous in length and possible substitution to the alkyls described above, but that contain at least one double or triple bond, respectively. The term —C_{x-y} alkenylene- refers to a substituted or unsubstituted alkenylene chain with from x to y carbons in the alkenylene chain. For example, —C₂₋₆ alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkynylene chain. For example, —C₂₋₆ alkynylene- may be selected from ethynylene, propynylene, butynylene, pentynylene, and hexynylene, any one of which is optionally substituted. An alkynylene chain may have one triple bond or more than one triple bond in the alkynylene chain.

The term “carbocycle” as used herein refers to a saturated, unsaturated or aromatic ring in which each atom of the ring is carbon. Carbocycle include 3- to 10-membered monocyclic rings and 6- to 12-membered bicyclic rings. Each ring of a bicyclic carbocycle may be selected from saturated, unsaturated, and aromatic rings. Bicyclic carbocycles may be fused, bridged or spiro-ring systems. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein.

“Cycloalkyl” refers to a stable fully saturated monocyclic or polycyclic hydrocarbon radical consisting solely of carbon and hydrogen atoms, which includes fused or bridged ring systems, and preferably having from three to twelve carbon atoms (i.e., C₃₋₁₂ cycloalkyl). In certain embodiments, a cycloalkyl comprises three to ten carbon atoms (i.e., C₃₋₁₀ cycloalkyl). In other embodiments, a cycloalkyl comprises five to seven carbon atoms (i.e., C₅₋₇ cycloalkyl). The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Cycloalkyl may be optionally substituted by one or more substituents such as those substituents described herein.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond (i.e., C₃₋₁₂ cycloalkenyl). In certain embodiments, a cycloalkenyl comprises three to ten carbon atoms (i.e., C₃₋₁₀ cycloalkenyl). In other embodiments, a cycloalkenyl comprises five to seven carbon atoms (i.e., C₅₋₇ cycloalkenyl). The cycloalkenyl may be attached to the rest of the molecule by a single bond. Examples of monocyclic cycloalkenyls include, e.g., cyclopentenyl, cyclohexenyl, cycloheptenyl, and cyclooctenyl. Cycloalkenyl may be optionally substituted by one or more substituents such as those substituents described herein.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Aryl may be optionally substituted by one or more substituents such as those substituents described herein.

A “C_{x-y} carbocycle” is meant to include groups that contain from x to y carbons in a ring For example, the term “C₃₋₆ carbocycle” can be a saturated, unsaturated or aromatic ring system that contains from 3 to 6 carbon atoms—any of which is optionally substituted as provided herein.

The term “heterocycle” as used herein refers to a saturated, unsaturated, non-aromatic or aromatic ring comprising one or more heteroatoms. Exemplary heteroatoms include N, O, Si, P, B, and S atoms. Heterocycles include 3- to 10-membered monocyclic rings and 6- to 12-membered bicyclic rings. Each ring of a bicyclic heterocycle may be selected from saturated, unsaturated, and aromatic rings. In some embodiments, the heterocycle comprises at least one heteroatom selected from oxygen, nitrogen, sulfur, or any combination thereof. In some embodiments, the heterocycle comprises at least one heteroatom selected from oxygen, nitrogen, or any combination thereof. In some embodiments, the heterocycle comprises at least one heteroatom selected from oxygen, sulfur, or any combination thereof. In some embodiments, the heterocycle comprises at least one heteroatom selected from nitrogen, sulfur, or any combination thereof. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Heterocycle may be optionally substituted by one or more substituents such as those substituents described herein. Bicyclic heterocycles may be fused, bridged or spiro-ring systems. In an exemplary embodiment, a heterocycle, e.g., pyridyl, may be fused to a saturated or unsaturated ring, e.g., cyclohexane, cyclopentane, or cyclohexene. Heterocycle may be optionally substituted by one or more substituents such as those substituents described herein.

“Heterocycloalkyl” refers to a stable 3- to 12-membered non-aromatic ring radical that comprises two to twelve carbon atoms and at least one heteroatom wherein each heteroatom may be selected from N, O, Si, P, B, and S atoms. In some embodiments, the heterocycloalkyl comprises at least one heteroatom selected from oxygen, nitrogen, sulfur, or any combination thereof. In some embodiments, the heterocycloalkyl comprises at least one heteroatom selected from oxygen, nitrogen, or any combination thereof. In some embodiments, the heterocycloalkyl comprises at least one heteroatom selected from oxygen, sulfur, or any combination thereof. In some embodiments, the heterocycloalkyl comprises at least one heteroatom selected from nitrogen, sulfur, or any combination thereof. The heterocycloalkyl may be selected from monocyclic or bicyclic, and fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Heterocycloalkyl may be optionally substituted by one or more substituents such as those substituents described herein.

The term “heteroaryl” refers to a radical derived from a 3- to 12-membered aromatic ring radical that comprises one to eleven carbon atoms and at least one heteroatom wherein each heteroatom may be selected from N, O, and S. In some embodiments, the heteroaryl comprises at least one heteroatom selected from oxygen, nitrogen, sulfur, or any combination thereof. In some embodiments, the heteroaryl comprises at least one heteroatom selected from oxygen, nitrogen, or any combination thereof. In some embodiments, the heteroaryl comprises at least one heteroatom selected from oxygen, sulfur, or any combination thereof. In some embodiments, the heteroaryl comprises at least one heteroatom selected from nitrogen, sulfur, or any combination thereof. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Heteroaryl includes aromatic single ring structures, preferably 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, thiazole, pyrazole, pyridine, pyrazine, pyridazine, and pyrimidine, and the like. Heteroaryl may be optionally substituted by one or more substituents such as those substituents described herein. Heteroaryl also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic. Heteroaryl may be optionally substituted by one or more substituents such as those substituents described herein.

An “X-membered heterocycle” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

“Alkoxy” refers to a radical bonded through an oxygen atom of the formula —O-alkyl, where alkyl is an alkyl chain as defined above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, and I). When an alkyl group is substituted with more than one halogen radicals, each halogen may be independently selected for example, 1-chloro,2-fluoroethane.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O) OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═NNH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═NNH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain. It will be understood by those skilled in the art that substituents can themselves be substituted, if appropriate.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases.

The phrase“pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms ““administer”, “administered”, “administers” and “administering” a compound should be understood to mean providing a compound of the invention or a prodrug of a compound of the invention to the individual in need.

As used herein, “treatment” or“treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

### Compounds

### Lactam Substituted 6-Imidazopyridazine IL-17A Modulators

In some aspects, the present disclosure provides a compound represented by the structure of Formula (I):

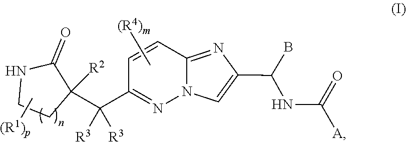

or a pharmaceutically acceptable salt thereof wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹,
   —N(R¹¹)S(O)₂R¹¹, —C(O)OR¹¹, —OC(O)R¹¹, —S(O)R¹¹, —S(O)₂R¹¹, —NO₂, —CN;
   C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂,
   —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹,
   —C(O)N(R¹¹)₂, —N(R¹¹)C(O)R¹¹, —C(O)OR¹¹, —OC(O)R¹¹, —NO₂, ═O, ═N(R¹¹), and —CN; and
   C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN;
B is selected from —C(H)(R⁵)₂ and C₃₋₁₀ carbocycle, wherein each C₃₋₁₀ carbocycle is optionally substituted with one or more substituents independently selected from:
   halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
   C₁₋₁₀ alkyl and C₃₋₁₀ carbocycle, any of which is optionally substituted with one or more substituents independently selected from halogen, —OR¹², —SR¹², —N(R¹²)₂,
   —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹²,
   —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁵ is independently selected at each occurrence from (i), (ii), and (iii):
   (iv) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
   (v) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³,
      —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (vi) C₃₋₁₀ carbocycle optionally substituted with one or more substituents independently selected from:
      halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂,
      —N(R¹³)C(O)R¹³, —C(O)OR¹³, —OC(O)R¹³, —S(O)R¹³, —S(O)₂R¹³, —NO₂, ═O, ═S, ═N(R¹³), and —CN;
each R¹ is independently selected at each occurrence from:
   halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN;
   C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN; and
   C₃₋₁₀ carbocycle optionally substituted with one or more substituents independently selected from halogen, —OR^{14A}, —SR^{14A}, —N(R^{14A})₂, —C(O)R^{14A}, —C(O)N(R^{14A})₂, —N(R^{14A})C(O)R^{14A}, —C(O)OR^{14A}, —OC(O)R^{14A}, —S(O)R^{14A}, —S(O)₂R^{14A}, —NO₂, ═O, ═S, ═N(R^{14A}), —CN; and C₁₋₁₀ alkyl optionally substituted with one or more substituents independently selected from halogen, —OR^{14A}, —SR^{14A}, —N(R^{14A})₂, —C(O)R^{14A}, —C(O)N(R^{14A})₂, —N(R^{14A})C(O)R^{14A}, —C(O)OR^{14A}, —OC(O)R^{14A}, —S(O)R^{14A}, —S(O)₂R^{14A}, —NO₂, ═O, ═S, ═N(R^{14A}), and —CN; or
two R¹ substituents may come together to form a C₃₋₆ carbocycle, wherein the C₃₋₆ carbocycle is optionally substituted with one or more substituents independently selected from:
   halogen, —OR^{18A}, —N(R^{18A})₂, —C(O)R^{18A}, —C(O)N(R^{18A})₂, —NO₂, ═O, ═S, ═N(R^{18A}), —CN; and C₁₋₁₀ alkyl optionally substituted with one or more substituents independently selected from halogen, —OR^{18A}, —N(R^{18A})₂, —C(O)R^{18A}, —C(O)N(R^{18A})₂, —NO₂, ═O, ═S, ═N(R^{18A}), and —CN;
R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN; or
each R⁴ is independently selected at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R^{17A})C(O)R^{17A}, —N(R¹⁷)S(O)₂R¹⁷, —C(O)OR¹⁷, —OC(O)R¹⁷, —S(O)R¹⁷, —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, and —CN;
R¹¹, R^{11A}, R¹², R¹³, R¹⁴, R^{14A}, R¹⁵, R¹⁶, R¹⁷, R^{17A}, and R^{18A}, are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2;
m is selected from 0, 1 and 2; and
p is selected from 0, 1, 2, 3, 4, 5, and 6.

In some embodiments, for the compound or salt of Formula (I), R¹ is optionally substituted C₃₋₁₀ carbocycle. In some embodiments, R¹ is optionally substituted C₃₋₁₀ saturated carbocycle. In some embodiments, R¹ is optionally substituted C₃₋₁₀ unsaturated carbocycle. In some embodiments, R¹ is selected from optionally substituted C₃ carbocycle, optionally substituted C₄ carbocycle, optionally substituted C₅ carbocycle, optionally substituted C₆ carbocycle, optionally substituted C₇ carbocycle, optionally substituted C₈ carbocycle, optionally substituted C₉ carbocycle, and optionally substituted C₁₀ carbocycle. In some embodiments, RI is selected from optionally substituted C₃₋₄ carbocycle, optionally substituted C₃₋₅ carbocycle, optionally substituted C₃₋₆ carbocycle, optionally substituted C₃₋₇ carbocycle, optionally substituted C₃₋₈ carbocycle, optionally substituted C₃₋₉ carbocycle, and optionally substituted C₃₋₁₀ carbocycle. In some embodiments, R¹ is optionally substituted C₃₋₁₀ carbocycle, wherein the optional substituents are as defined herein. In some embodiments, R¹ is optionally substituted C₃₋₁₀ carbocycle, wherein the optional substituents are as defined in Formula (I), (II), (II-a), or (II-b).

In some embodiments, for the compound or salt of Formula (I), R¹ is C₃₋₁₀ carbocycle optionally substituted with one or more substituents independently selected from halogen, —OR^{14A}, —SR^{14A}, —N(R^{14A})₂, —C(O)R^{14A}, —C(O)N(R^{14A})₂, —N(R^{14A})C(O)R^{14A}, —C(O)OR^{14A}, —OC(O)R^{14A}, —S(O)R^{14A}, —S(O)₂R^{14A}, —NO₂, ═O, ═S, ═N(R^{14A}), —CN; and C₁₋₁₀ alkyl optionally substituted with one or more substituents independently selected from halogen, —OR^{14A}, —SR^{14A}, —N(R^{14A})₂, —C(O)R^{14A}, —C(O)N(R^{14A})₂, —N(R^{14A})C(O)R^{14A}, —C(O)OR^{14A}, —OC(O)R^{14A}, —S(O)R^{14A}, —S(O)₂R^{14A}, —NO₂, ═O, ═S, ═N(R^{14A}), and —CN; and R^{14A} is selected from hydrogen, C₁₋₆ alkyl, C₁₋₆ haloalkyl, C₃₋₆ carbocycle, and 3- to 6-membered heterocycle.

In some embodiments, for the compound or salt of Formula (I), R¹ is C₃₋₁₀ carbocycle optionally substituted with one or more substituents independently selected from halogen, —OR^{14A}, —SR^{14A}, —N(R^{14A})₂, —C(O)R^{14A}, —C(O)N(R^{14A})₂, —N(R^{14A})C(O)R^{14A}, —C(O)OR^{14A}, —OC(O)R^{14A}, —S(O)R^{14A}, —S(O)₂R^{14A}, —NO₂, ═O, ═S, ═N(R^{14A}), —CN; and C₁₋₁₀ alkyl optionally substituted with one or more substituents independently selected from halogen, —OR^{14A}, SR^{14A}, —N(R^{14A})₂, —C(O)R^{14A}, —C(O)N(R^{14A})₂, —N(R^{14A})C(O)R^{14A}, —C(O)OR^{14A}, —OC(O)R^{14A}, —S(O)R^{14A}, —S(O)₂R^{14A}, —NO₂, ═O, ═S, ═N(R^{14A}), and —CN. In some embodiments, R¹ is C₃₋₆ carbocycle optionally substituted with one or more substituents independently selected from halogen, —OR^{14A}, —N(R^{14A})₂, —C(O)R^{14A}, —NO₂, —CN; and C₁₋₆ alkyl optionally substituted with one or more substituents independently selected from halogen, —OR^{14A}, —N(R^{14A})₂, —C(O)R^{14A}, —NO₂, and —CN. In some embodiments, R¹ is C₃₋₆ cycloalkyl optionally substituted with one or more substituents independently selected from halogen, —OR^{14A}, —N(R^{14A})₂, —C(O)R^{14A}, —NO₂, —CN, C₁₋₆ alkyl, and C₁₋₆haloalkyl. In some embodiments, R¹ is

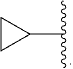 [SMILES: *C1CC1]

In some embodiments, for the compound or salt of Formula (I), two R¹ substituents may come together to form a C₃₋₆ carbocycle, wherein the C₃₋₆ carbocycle is optionally substituted with one or more substituents independently selected from: halogen, —OR^{18A}, —N(R^{18A})₂, —C(O)R^{18A}, —C(O)N(R^{18A})₂, —NO₂, ═O, ═S, ═N(R^{18A}), —CN; and C₁₋₁₀ alkyl optionally substituted with one or more substituents independently selected from halogen, —OR^{18A}, —N(R^{18A})₂, —C(O)R^{18A}, —C(O)N(R^{18A})₂, —NO₂, ═O, ═S, ═N(R^{18A}), and —CN; and R^{18A} is selected from hydrogen, C₁₋₆ alkyl, and C₁₋₆haloalkyl.

In some embodiments, for the compound or salt of Formula (I), two R¹ substituents may come together to form a C₃₋₆ carbocycle, wherein the C₃₋₆ carbocycle is optionally substituted with one or more substituents independently selected from: halogen, —OR^{18A}, —N(R^{18A})₂, —C(O)R^{18A}, —C(O)N(R^{18A})₂, —NO₂, ═O, ═S, ═N(R^{18A}), —CN; and C₁₋₁₀ alkyl optionally substituted with one or more substituents independently selected from halogen, —OR^{18A}, —N(R^{18A})₂, —C(O)R^{18A}, —C(O)N(R^{18A})₂, —NO₂, ═O, ═S, ═N(R^{18A}), and —CN. In some embodiments, two R¹ substituents may come together to form a C₃₋₆ cycloalkyl optionally substituted with one or more substituents independently selected from: halogen, —OR^{18A}, —N(R^{18A})₂, —C(O)R^{18A}, —C(O)N(R^{18A})₂, —NO₂, —CN, C₁₋₆ alkyl, and C₁₋₆haloalkyl. In some embodiments, two R¹ substituents on the same carbon atom may come together to form a C₃₋₆ cycloalkyl optionally substituted with one or more substituents independently selected from: halogen, —OR^{18A}, —N(R^{18A})₂, —C(O)R^{18A}, —C(O)N(R^{18A})₂, —NO₂, —CN, C₁₋₆ alkyl, and C₁₋₆ haloalkyl (For example two R¹ substituents on the same carbon atom may come together to form a C₃ cycloalkyl, wherein

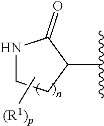

is represented by

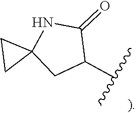 [SMILES: *C1CC2(CC2)NC1=O]

In some embodiments, two R¹ substituents on adjacent carbon atoms may come together to form a C₃₋₆ cycloalkyl optionally substituted with one or more substituents independently selected from: halogen, —OR^{18A}, —N(R^{18A})₂, —C(O)R^{18A}, —C(O)N(R^{18A})₂, —NO₂, —CN, C₁₋₆ alkyl, and C₁₋₆ haloalkyl (For example, two R¹ substituents on adjacent carbon atoms may come together to form a C₃ cycloalkyl, wherein

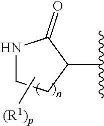

is represented by

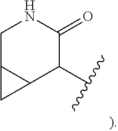 [SMILES: *C1C(=O)NCC2CC21]

In some embodiments, two R¹ substituents on different carbon atoms may come together to form a C₃₋₆ cycloalkyl optionally substituted with one or more substituents independently selected from: halogen, —OR^{18A}, —N(R^{18A})₂, —C(O)R^{18A}, —C(O)N(R^{18A})₂, —NO₂, —CN, C₁₋₆ alkyl, and C₁₋₆haloalkyl (For example, two R¹ substituents on different carbon atoms may come together to form a C₄ cycloalkyl, wherein

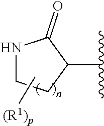

is represented by

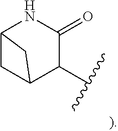 [SMILES: *C1C(=O)NC2CC1C2]

In some embodiments, two R¹ substituents may come together to form,

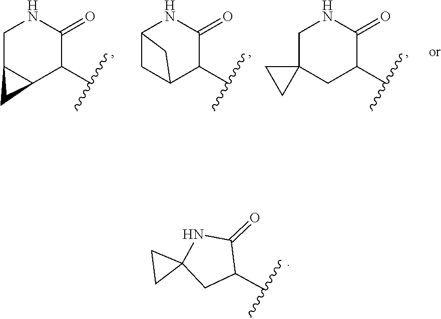

In some embodiments, for the compound or salt of Formula (I), R⁴ is —N(R^{17A})C(O)R^{17A}, and each R^{17A} is selected from hydrogen, C₁₋₆ alkyl, and C₁₋₆haloalkyl.

In some embodiments, for the compound or salt of Formula (I), A is 5- to 6-membered heteroaryl, any of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹,
—OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein C₃₋₁₀ carbocycle and 3- to 10-membered heterocycle are each optionally substituted with one or more substituents selected from: halogen, —OR¹¹,
—N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

In some embodiments, for the compound or salt Formula (I), A is 5- to 6-membered heteroaryl, any of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

In some embodiments, for the compound or salt Formula (I), A is 5- to 6-membered heteroaryl, any of which is optionally substituted with one or more substituents independently selected from:
C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
C₃₋₁₀ carbocycle optionally substituted with one or more substituents independently selected from halogen, —OR^{11A}, SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

In some embodiments, for the compound or salt Formula (I), A is 5- to 6-membered heteroaryl, any of which is optionally substituted with one or more substituents independently selected from:
C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

In some embodiments, for the compound or salt Formula (I), A is 5-membered heteroaryl, optionally substituted with one or more substituents independently selected from:
C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

In some embodiments, for the compound or salt of Formula (I), A is selected from pyrazolyl, imidazolyl, oxazolyl, isoxazolyl, oxadiazolyl, optionally substituted with C₃₋₆ carbocycle optionally substituted with one or more substituents independently selected from halogen, —OR^{11A}, —N(R^{11A})₂, —C(O)R^{11A}, —NO₂, —CN, C₁₋₆ alkyl, and C₁₋₆ haloalkyl. In some embodiments, A is selected from pyrazolyl, imidazolyl, oxazolyl, isoxazolyl, oxadiazolyl, optionally substituted with C₃-6 saturated carbocycle optionally substituted with one or more substituents independently selected from halogen, —OR^{11A}, —N(R^{11A})₂, —C(O)R^{11A}, —NO₂, —CN, C₁₋₆ alkyl, and C₁₋₆ haloalkyl.

In some embodiments, for the compound or salt of Formula (I), A is selected from pyrazolyl, imidazolyl, oxazolyl, isoxazolyl, oxadiazolyl, optionally substituted with C₃₋₆ cycloalkyl optionally substituted with one or more substituents independently selected from halogen, —OR^{11A}, —N(R^{11A})₂, —C(O)R^{11A}, —NO₂, —CN, C₁₋₆ alkyl, and C₁₋₆ haloalkyl. In some embodiments, A is selected from pyrazolyl, imidazolyl, oxazolyl, isoxazolyl, oxadiazolyl, optionally substituted with cyclopropyl, cyclobutyl, cyclopentyl, and cyclohexyl, each of which is optionally substituted with one or more substituents independently selected from halogen, —OR^{11A}, —N(R^{11A})₂, —C(O)R^{11A}, —NO₂, —CN, C₁₋₆ alkyl, and C₁₋₆ haloalkyl. In some embodiments, A is selected from

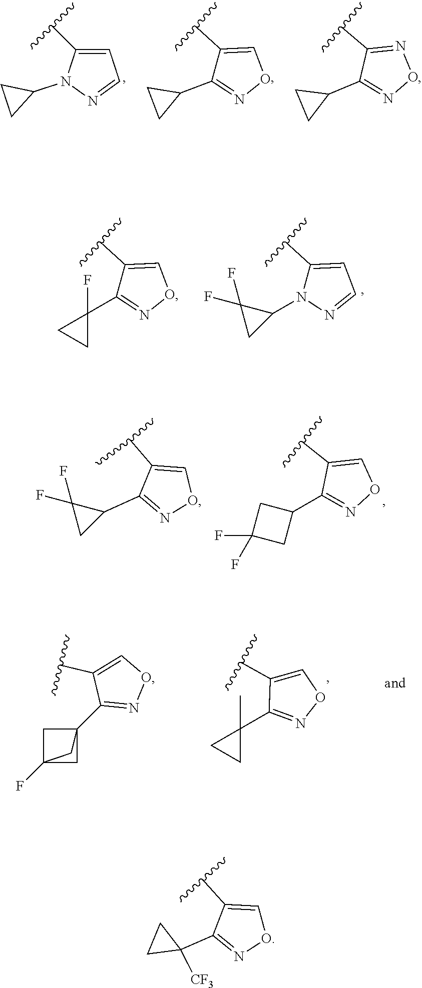

In some embodiments A is selected from

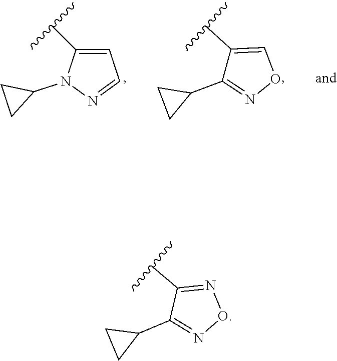

In some embodiments, A is selected from

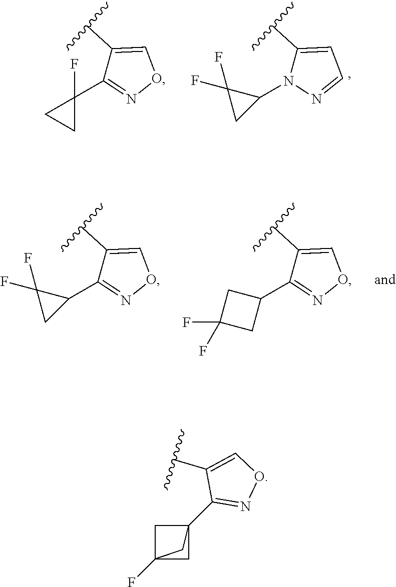

In some embodiments,

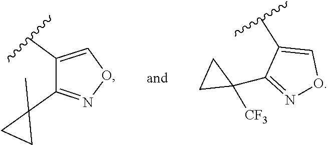

In some embodiments, for the compound or salt of Formula (I), R^{11A}, R^{14A}, R^{17A}, and R^{18A}, are each independently selected from
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN.

In some aspects, the present disclosure provides a compound represented by the structure of Formula (II):

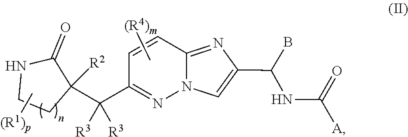

or a pharmaceutically acceptable salt thereof wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
   C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂,
   —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN;
B is selected from —C(H)(R⁵)₂ and C₃₋₁₀ carbocycle, wherein each C₃₋₁₀ carbocycle is optionally substituted with one or more substituents independently selected from:
   halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
   C₁₋₁₀ alkyl and C₃₋₁₀ carbocycle, any of which is optionally substituted with one or more substituents independently selected from halogen, —OR¹², —SR¹², —N(R¹²)₂,
   —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹²,
   —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁵ is independently selected at each occurrence from (i), (ii), and (iii):
   (i) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³,
      —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (iii) C₃₋₁₀ carbocycle optionally substituted with one or more substituents independently selected from:
      halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂,
      —N(R¹³)C(O)R¹³, —C(O)OR¹³, —OC(O)R¹³, —S(O)R¹³, —S(O)₂R¹³, —NO₂, ═O, ═S, ═N(R¹³), and —CN;
each R¹ is independently selected at each occurrence from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴,
   —S(O)₂R¹⁴, —NO₂, ═O, ═S, ═N(R¹⁴), —CN; and C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN;
R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN; or
each R⁴ is independently selected at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷,
   —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN;
R¹¹, R¹², R¹³, R¹⁴, R¹⁵, R¹⁶, and R¹⁷ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2;
m is selected from 0, 1 and 2; and
p is selected from 0, 1, 2, 3, 4, 5, and 6.

In some embodiments, for the compound or salt of Formula (II), n is 1.

In some aspects, Formula (II) is represented by the structure of Formula (II-a):

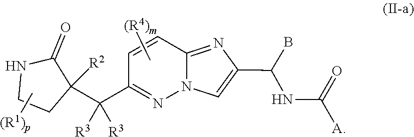

In some aspects, Formula (II) is represented by the structure of Formula (II-a):

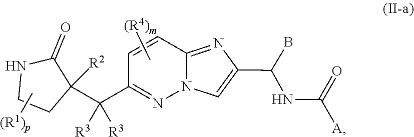

wherein R¹, R², R³, R⁴, A, B, p and m are each defined as in Formula (I).

In some aspects, Formula (II) is represented by the structure of Formula (II-a):

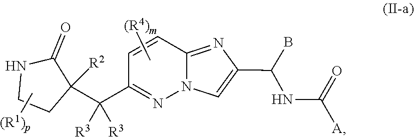

wherein R¹, R², R³, R⁴, A, B, p and m are each defined as in Formula (II).

In some embodiments, for the compound or salt of Formula (II), n is 2

In some aspects, Formula (II) is represented by the structure of Formula (II-b):

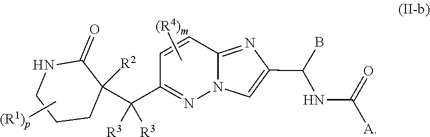

In some aspects, Formula (II) is represented by the structure of Formula (II-b):

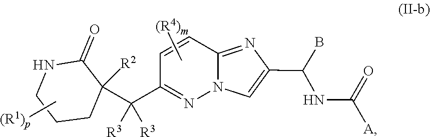

wherein R¹, R², R³, R⁴, A, B, p and m are each defined as in Formula (I).

In some aspects, Formula (II) is represented by the structure of Formula (II-b):

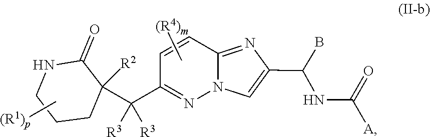

wherein R¹, R², R³, R⁴, A, B, p and m are each defined as in Formula (II).

In some embodiments, for the compound or salt of Formula (I), (II), (II-a), or (II-b), p is selected from 0, 1, 2, 3, 4, 5, and 6. In some embodiments, p is selected from 1, 2, 3, 4, 5, and 6. In some embodiments, p is selected from 0, 1, 2, 3, 4, and 5. In some embodiments, p is selected from 0, 1, 2, 3, and 4. In some embodiments, p is selected from 0, 1, 2, and 3. In some embodiments, p is selected from 0, 1, and 2. In some embodiments, p is selected from 0 and 1. In some embodiments, p is selected from 1, 2, 3, 4 and 5. In some embodiments, p is selected from 2, 3, 4 and 5. In some embodiments, p is selected from 3, 4 and 5. In some embodiments, p is selected from 4 and 5. In some embodiments, p is 0. In some embodiments, p is 1. In some embodiments, p is 2. In some embodiments, p is 3. In some embodiments, p is 4. In some embodiments, p is 5. In some embodiments, p is 6.

In some embodiments, for the compound or salt of Formula (I), (II), (II-a), or (II-b), each R¹ is selected from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN; and C₁₋₆ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN. In some embodiments, each R¹ is selected from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), and —CN. In some embodiments, each R¹ is selected from C₁₋₆ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN. In some embodiments, each R¹ is selected from halogen, —OR¹⁴, —N(R¹⁴)₂, —NO₂, —CN, C₁₋₆ alkyl, and C₁₋₆ haloalkyl. In some embodiments, each R¹ is selected from halogen and —CF₃. In some embodiments, each R¹ is —CF₃.

In some embodiments, for the compound or salt of Formula (I), (II), (II-a), or (II-b), each R¹ is selected from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN; and C₁₋₆ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN. In some embodiments, R¹ is selected from halogen, —OR¹⁴, —SR¹⁴, —N(R¹⁴)₂, —C(O)R¹⁴, —NO₂, —CN; and C₁₋₆ alkyl optionally substituted with one or more substituents independently selected from halogen, —OR¹⁴, —SR¹⁴, —N(R¹⁴)₂, —C(O)R¹⁴, —NO₂, —CN. In some embodiments, each R¹ is independently selected at each occurrence from halogen, C₁₋₆ alkyl, and C₁₋₆ haloalkyl. In some embodiments, each R¹ is independently selected at each occurrence from methyl, ethyl,

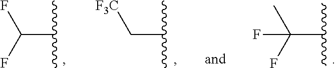

In some embodiments, for the compound or salt of Formula (I), (II), (II-a), or (II-b), R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN, and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN. In some embodiments, R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, and —CN. In some embodiments, R² is selected from C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN. In some embodiments, R² is selected from hydrogen, halogen, —OR¹⁵, —N(R¹⁵)₂, —C(O)R¹⁵, —NO₂, —CN, C₁₋₃ alkyl, and C₁₋₃ haloalkyl. In some embodiments, R² is selected from hydrogen, halogen, and C₁₋₃ alkyl. In some embodiments, R² is selected from hydrogen, halogen, methyl, and ethyl. In some embodiments, R² is hydrogen.

In some embodiments, for the compound or salt of Formula (I), (II), (II-a), or (II-b), R² is selected from hydrogen, C₁₋₃ alkyl, and C₁₋₃ haloalkyl.

In some embodiments, for the compound or salt of Formula (I), (II), (II-a), or (II-b), each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN, and C₁₋₆ alkyl optionally substituted with one or more substituents independently selected from halogen, —OR¹⁶, —SR¹⁶, —N(R¹⁶)₂, —C(O)R¹⁶, and —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN. In some embodiments, each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, and —CN. In some embodiments, each R³ is independently selected at each occurrence from C₁₋₆ alkyl optionally substituted with one or more substituents independently selected from halogen, —OR¹⁶, —SR¹⁶, —N(R¹⁶)₂, —C(O)R¹⁶, and —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN. In some embodiments, each R³ is independently selected at each occurrence from hydrogen and C₁₋₆ alkyl optionally substituted with one or more substituents independently selected from halogen,
—OR¹⁶, —SR¹⁶, —N(R¹⁶)₂, —C(O)R¹⁶, and —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN. In some embodiments, each R³ is independently selected at each occurrence from hydrogen, halogen, —OR¹⁶, —N(R¹⁶)₂, —NO₂, —CN, C₁₋₃ alkyl, and C₁₋₃ haloalkyl. In some embodiments, each R³ is independently selected at each occurrence from hydrogen and methyl. In some embodiments, each R³ is hydrogen.

In some embodiments, for the compound or salt of Formula (I), (II), (II-a), or (II-b), R³ is independently selected at each occurrence from hydrogen, methyl, ethyl, and

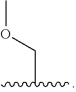 [SMILES: *COC]

In some embodiments, for the compound or salt of Formula (I), (II), (II-a), or (II-b), m is 2.

In some embodiments, for the compound or salt of Formula (I), (II), (II-a), or (II-b), m is 1.

In some embodiments, for the compound or salt of Formula (I), (II), (II-a), or (II-b), m is 0.

In some embodiments, for the compound or salt of Formula (I), (II), (II-a), or (II-b), each R⁴ is independently selected from at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN, and C₁₋₆ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —C(O)OR¹⁷, —OC(O)R¹⁷, —S(O)R¹⁷, —S(O)₂R¹⁷, —NO₂, ═O, ═S, ═N(R¹⁷), and —CN. In some embodiments, each R⁴ is independently selected from at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, and —CN. In some embodiments, each R⁴ is independently selected from at each occurrence from C₁₋₆ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —C(O)OR¹⁷, —OC(O)R¹⁷, —S(O)R¹⁷, —S(O)₂R¹⁷, —NO₂, ═O, ═S, ═N(R¹⁷), and —CN. In some embodiments, each R⁴ is selected from halogen, —OR¹⁷, —N(R¹⁷)₂, —NO₂, —CN, C₁₋₃ alkyl, and C₁₋₃ haloalkyl. In some embodiments, each R⁴ is selected from chloro, fluoro, methyl, ethyl, and —CN.

In some embodiments, for the compound or salt of Formula (I), (II), (II-a), or (II-b), m is 1; and R⁴ is selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, and —CN. In some embodiments, R⁴ is selected from halogen, —OR¹⁷, —N(R¹⁷)₂, —C(O)R¹⁷, —S(O)₂R¹⁷, and —CN; and R¹⁷ is selected at each occurrence from hydrogen; C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₆ carbocycle and 3- to 6-membered heterocycle, wherein each C₃₋₆ carbocycle and 3- to 6-membered heterocycle; and C₃₋₆ carbocycle and 3- to 6-membered heterocycle optionally substituted with one or more substituents independently selected from halogen, —OH, —O—C₁₋₆ alkyl, —O—C₁₋₆ haloalkyl, —NH₂, —NO₂, ═O, and —CN. In some embodiments, R⁴ is selected from —Cl, —CN,

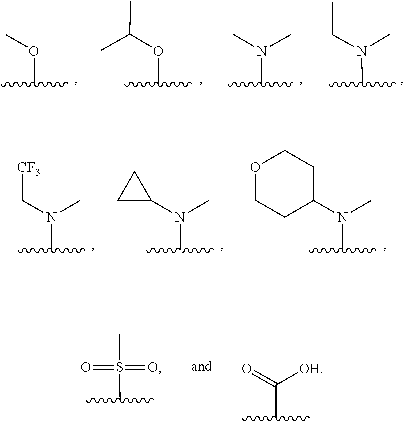

In some embodiments, R⁴ is C₁₋₆alkyl optionally substituted with one or more substituents independently selected from halogen, —OR¹⁷, and —CN; and R¹⁷ is selected at each occurrence from halogen, C₁₋₆ alkyl, and C₁₋₆haloalkyl. In some embodiments, R⁴ is selected from methyl,

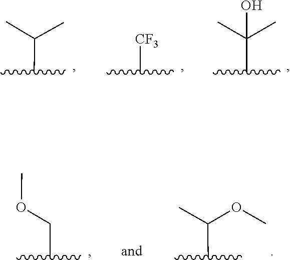

In some embodiments, for the compound or salt of Formula (I), (II), (II-a), or (II-b), A is optionally substituted C₃₋₆ carbocycle. In some embodiments, Ais optionally substituted saturated C₃₋₆ carbocycle. In some embodiments, A is optionally substituted unsaturated C₃₋₆ carbocycle. In some embodiments, A is selected from optionally substituted C₃₋₅ carbocycle, optionally substituted C₃₋₄ carbocycle, optionally substituted C₄₋₆ carbocycle, and optionally substituted C₅₋₆ carbocycle. In some embodiments, A is selected from optionally substituted C₃ carbocycle, optionally substituted C₄ carbocycle, optionally substituted C₅ carbocycle, and optionally substituted C₆ carbocycle.

In some embodiments, for the compound or salt of Formula (I), (II), (II-a), or (II-b), A is optionally substituted 5- to 6-membered heteroaryl. In some embodiments, A is optionally substituted 5- to 6-membered heteroaryl comprising at least one heteroatom selected from nitrogen, oxygen, and sulfur. In some embodiments, A is optionally substituted 5- to 6-membered heteroaryl comprising at least one nitrogen or oxygen heteroatom. In some embodiments, A is optionally substituted 5- to 6-membered heteroaryl comprising at least one nitrogen or sulfur heteroatom. In some embodiments, A is optionally substituted 5- to 6-membered heteroaryl comprising at least one sulfur or oxygen heteroatom. In some embodiments, A is optionally substituted 5- to 6-membered heteroaryl comprising at least one sulfur heteroatom. In some embodiments, A is optionally substituted 5- to 6-membered heteroaryl comprising at least one oxygen heteroatom. In some embodiments, A is optionally substituted 5- to 6-membered heteroaryl comprising at least one nitrogen heteroatom.

In some embodiments, for the compound or salt of Formula (I), (II), (II-a), or (II-b)), A is selected from a saturated C₃₋₆ carbocycle and a 5-membered heteroaryl, any of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹,
—OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein C₃₋₁₀ carbocycle and 3- to 10-membered heterocycle are each optionally substituted with one or more substituents selected from: halogen, —OR¹¹,
—N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN.

In some embodiments, for the compound or salt of Formula (I), (II), (II-a), or (II-b), A is 5- to 6-membered heteroaryl, each of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹,
—OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein C₃₋₁₀ carbocycle and 3- to 10-membered heterocycle are each optionally substituted with one or more substituents selected from: halogen, —OR¹¹,
—N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN.

In some embodiments, for the compound or salt of Formula (I), (II), (II-a), or (II-b), A is 5-membered heteroaryl optionally substituted with one or more substituents independently selected from:
halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN; and
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN, C₃₋₁₀ carbocycle and 3- to 10-membered heterocycle; wherein C₃₋₁₀ carbocycle and 3- to 10-membered heterocycle are each optionally substituted with one or more substituents selected from: —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

In some embodiments, for the compound or salt of Formula (I), (II), (II-a), or (II-b), A is 5- to 6-membered heteroaryl optionally substituted with one or more substituents independently selected from: halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN. In some embodiments, Ais 5- to 6-membered heteroaryl optionally substituted with one or more substituents independently selected from: C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN, C₃₋₆ carbocycle and 3- to 6-membered heterocycle; wherein the C₃₋₆ carbocycle and 3- to 6-membered heterocycle are each optionally substituted with one or more substituents selected from: —OR¹¹, —SR¹¹, —N(R¹¹)₂, —C(O)R¹¹,
—C(O)N(R¹¹)₂, —C(O)OR¹¹, —OC(O)R¹¹, —NO₂, and —CN.

In some embodiments, for the compound or salt of Formula (I), (II), (II-a), or (II-b), A is selected from 5- to 6-membered heteroaryl and C₃₋₆ carbocycle, any of which is optionally substituted with one or more substituents independently selected from C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN. In some embodiments, A is 5- to 6-membered heteroaryl optionally substituted with one or more substituents independently selected from C₃₋₁₀carbocycle optionally substituted with one or more substituents independently selected from halogen, —OR^{11A}, SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN. In some embodiments, A is 5-membered heteroaryl optionally substituted with C₃₋₁₀ carbocycle optionally substituted with one or more substituents independently selected from halogen, —OR^{11A}, —N(R^{11A})₂, —C(O)R^{11A}, —NO₂, —CN; and C₁₋₁₀ alkyl optionally substituted with one or more substituents selected from: halogen, —OR^{11A}, —N(R^{11A})₂, —C(O)R^{11A}, —NO₂, and —CN.

In some embodiments, for the compound or salt of Formula (I), (II), (II-a), or (II-b), A is selected from pyrazolyl, imidazolyl, oxazolyl, isoxazolyl, oxadiazolyl, triazolyl, and tetrazolyl, any one of which is optionally substituted with C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN. In some embodiments, A is selected from pyrazolyl and oxadiazolyl, each of which is optionally substituted with C₁₋₆ alkyl optionally substituted with one or more substituents independently selected from halogen, —OR¹¹, —N(R¹¹)₂, —C(O)R¹¹, —NO₂, and —CN. In some embodiments, A is selected from pyrazolyl and oxadiazolyl, each of which is optionally substituted with methyl, ethyl, isopropyl, or propyl. In some embodiments, A is selected from:

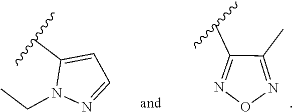

In some embodiments, A is

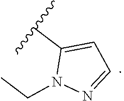 [SMILES: *c1ccnn1CC]

In some embodiments, A is

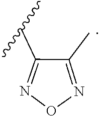 [SMILES: *c1nonc1C]

In some embodiments, for the compound or salt of Formula (I), (II), (II-a), or (II-b), A is selected from thiophenyl, thiadiazolyl, pyrrolyl, pyrazolyl, imidazolyl, oxazolyl, isoxazolyl, oxadiazolyl, triazolyl, and tetrazolyl, any one of which is optionally substituted with one or more substituents independently selected from:
halogen, —OR¹¹, —SR¹¹, —N(R¹¹)₂, —C(O)R¹¹, —C(O)N(R¹¹)₂, —C(O)OR¹¹, —OC(O)R¹¹,
—NO₂, —CN; and
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹,
—NO₂, —CN, C₃₋₁₀ carbocycle, and 3- to 10-membered heterocycle; wherein the C₃₋₁₀ carbocycle and 3- to 10-membered heterocycle are each optionally substituted with one or more substituents selected from: —OR¹¹, —SR¹¹, —N(R¹¹)₂, —C(O)R¹¹, —C(O)N(R¹¹)₂, —C(O)OR¹¹,
—OC(O)R¹¹, —NO₂, and —CN.

In some embodiments, for the compound or salt of Formula (I), (II), (II-a), or (II-b), A is selected from thiophenyl, thiadiazolyl, pyrrolyl, pyrazolyl, imidazolyl, oxazolyl, isoxazolyl, oxadiazolyl, and triazolyl, any one of which is optionally substituted with one or more substituents independently selected from halogen, —OR¹¹, —N(R¹¹)₂, —NO₂, —CN; and C₁₋₆ alkyl optionally substituted with one or more substituents independently selected from halogen, —OR¹¹, —N(R¹¹)₂, —NO₂, —CN, C₃₋₆ carbocycle and 3- to 6-membered heterocycle. In some embodiments, A is selected from thiophenyl, thiadiazolyl, pyrrolyl, pyrazolyl, imidazolyl, oxazolyl, isoxazolyl, oxadiazolyl, and triazolyl, any one of which is optionally substituted with one or more substituents independently selected from —OR¹¹, —N(R¹¹)₂, —CN; and C₁₋₃ alkyl optionally substituted with one or more substituents independently selected from halogen, —OR¹¹, —N(R¹¹)₂, and C₃₋₆ cycloalkyl. In some embodiments, A is selected from:

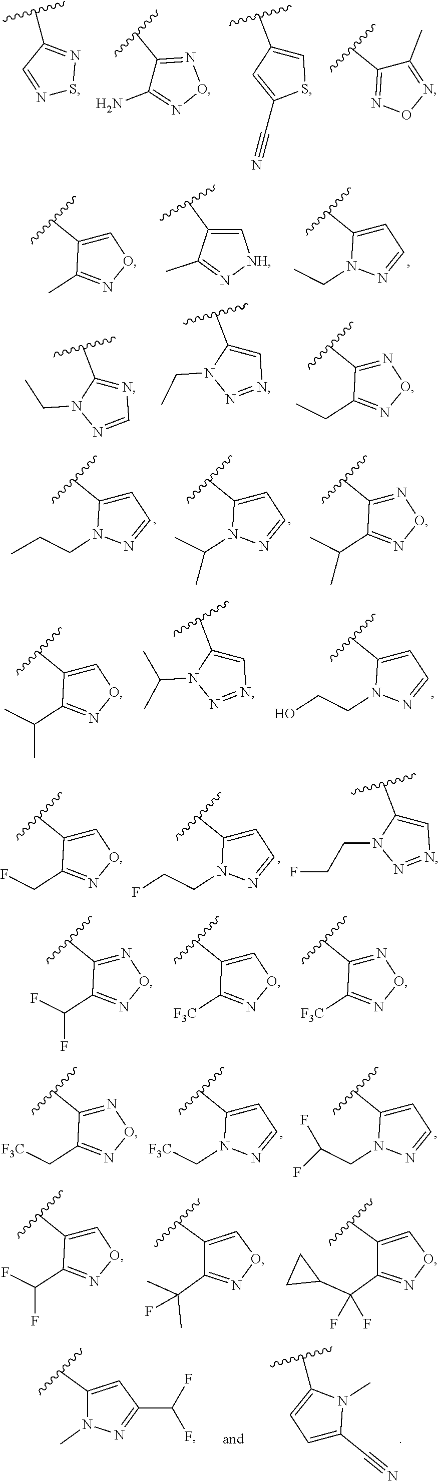

In some embodiments, for the compound or salt of Formula (I), (II), (II-a), or (II-b), A is optionally substituted C₃₋₆ carbocycle. In some embodiments, the optionally substituted C₃₋₆ carbocycle of A is selected from optionally substituted C₄₋₆ spirocyclic carbocycle and optionally substituted C₄₋₆ bridged carbocycle. In some embodiments, the optional substituents of the optionally substituted C₄₋₆ spirocyclic carbocycle and optionally substituted C₄₋₆ bridged carbocycle of A, are as defined as herein. In some embodiments, the optional substituents of the optionally substituted C₄₋₆ spirocyclic carbocycle and optionally substituted C₄₋₆ bridged carbocycle of A, are as defined as in Formula (II), (II-a), or (II-b).

In some embodiments, for the compound or salt of Formula (I), (II), (II-a), or (II-b), A is C₃₋₆ carbocycle optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN; and C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN. In some embodiments, optionally substituted C₃₋₆ carbocycle of A is selected from spirocyclic C₄₋₆ carbocycle and bridged C₄₋₆ carbocycle, each of which is optionally substituted with one or more substituents independently selected from halogen, —OR¹¹, —N(R¹¹)₂—NO₂, —CN, C₁₋₆ alkyl, and C₁₋₆haloalkyl. In some embodiments, A is

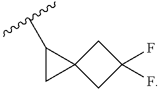 [SMILES: *C1CC12CC(F)(F)C2]

In some embodiments, for the compound or salt of Formula (I), (II), (II-a), or (II-b), B is —C(H)(R⁵)₂. In some embodiments, each R⁵ is independently selected at each occurrence from halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), and —CN. In some embodiments, each R⁵ is independently selected at each occurrence from C₁₋₆ alkyl optionally substituted with one or more substituents independently selected from halogen, —OR¹³, —SR¹³,
—N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), and —CN. In some embodiments, each R⁵ is independently selected at each occurrence from C₃₋₆ carbocycle optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), and —CN. In some embodiments, each R⁵ is independently selected at each occurrence from C₃₋₆ carbocycle optionally substituted with one or more substituents C₁₋₆ alkyl optionally substituted with one or more substituents independently selected from halogen, —OR¹³,
—SR¹³, —N(R¹³)₂, —C(O)R¹³, —C(O)N(R¹³)₂, —N(R¹³)C(O)R¹³, —C(O)OR¹³, —OC(O)R¹³, —S(O)R¹³, —S(O)₂R¹³, —NO₂, ═O, ═S, ═N(R¹³), and —CN.

In some embodiments, for the compound or salt of Formula (I), (II), (II-a), or (II-b), each R⁵ of —C(H)(R⁵)₂ is independently selected from:
halogen, —OR¹³, —N(R¹³)₂, —C(O)R¹³, —C(O)OR¹³, —NO₂, —CN;
C₁₋₆ alkyl optionally substituted with one or more substituents independently selected from halogen, —OR¹³, —N(R¹³)₂, —C(O)R¹³, —C(O)OR¹³, —NO₂, ═O, ═S, ═N(R¹³), and —CN; and
C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl.

In some embodiments, for the compound or salt of Formula (I), (II), (II-a), or (II-b), R⁵ is optionally substituted C₃₋₁₀ carbocycle. In some embodiments, R⁵ is selected from optionally substituted C₃₋₄ carbocycle, optionally substituted C₃₋₅ carbocycle, optionally substituted C₃₋₆ carbocycle, optionally substituted C₃₋₇ carbocycle, optionally substituted C₃₋₈ carbocycle, and optionally substituted C₃₋₉ carbocycle. In some embodiments, R⁵ is selected from optionally substituted C₄₋₁₀ carbocycle, optionally substituted C₅₋₁₀ carbocycle, optionally substituted C₆₋₁₀ carbocycle, optionally substituted C₇₋₁₀ carbocycle, optionally substituted C₈₋₁₀ carbocycle, and optionally substituted C₉₋₁₀ carbocycle. In some embodiments, R⁵ is an optionally substituted C₃₋₁₀ saturated carbocycle. In some embodiments, R⁵ is an optionally substituted C₃₋₁₀ unsaturated carbocycle. In some embodiments, R⁵ is selected from an optionally substituted C₃₋₆ carbocycle and optionally substituted C₆₋₁₀ carbocycle. In some embodiments, R⁵ is selected from an optionally substituted C₃₋₈ monocyclic carbocycle and optionally substituted C₆₋₁₀ bicyclic carbocycle.

In some embodiments, for the compound or salt of Formula (I), (II), (II-a), or (II-b), each R⁵ of —C(H)(R⁵)₂ is independently selected from C₃₋₆ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl. In some embodiments, —CH(R⁵)₂ is

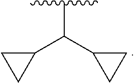 [SMILES: *C(C1CC1)C1CC1]

In some embodiments, for the compound or salt of Formula (I), (II), (II-a), or (II-b), each R⁵ of —C(H)(R⁵)₂ is independently selected from C₁₋₆ alkyl optionally substituted with one or more substituents independently selected from halogen, —OR¹³, —N(R¹³)₂, —C(O)R¹³, —C(O)OR¹³, —NO₂, ═O, ═S, ═N(R¹³), and —CN; and C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl. In some embodiments, each R⁵ of —C(H)(R⁵)₂ is independently selected from halogen, —OR¹³, —N(R¹³)₂, —C(O)R¹³, —C(O)OR¹³, —NO₂, ═O, ═S, ═N(R¹³), and —CN. In some embodiments, —CH(R⁵)₂ is selected from

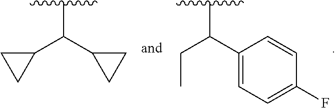 [SMILES: *C(C1CC1)C1CC1 ; *C(CC)c1ccc(F)cc1]

In some embodiments, B is selected from

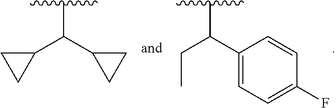 [SMILES: *C(C1CC1)C1CC1 ; *C(CC)c1ccc(F)cc1]

In some embodiments, B is

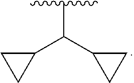 [SMILES: *C(C1CC1)C1CC1]

In some embodiments, B is

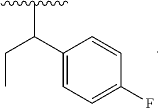 [SMILES: *C(CC)c1ccc(F)cc1]

In some embodiments, for the compound or salt of Formula (I), (II), (II-a), or (II-b), B is optionally substituted C₃₋₁₀ carbocycle. In some embodiments, B is selected from optionally substituted C₃₋₄ carbocycle, optionally substituted C₃₋₅ carbocycle, optionally substituted C₃₋₆ carbocycle, optionally substituted C₃₋₇ carbocycle, optionally substituted C₃₋₈ carbocycle, and optionally substituted C₃₋₉ carbocycle. In some embodiments, B is selected from optionally substituted C₄₋₁₀ carbocycle, optionally substituted C₅₋₁₀ carbocycle, optionally substituted C₆₋₁₀ carbocycle, optionally substituted C₇₋₁₀ carbocycle, optionally substituted C₈₋₁₀ carbocycle, and optionally substituted C₉₋₁₀ carbocycle. In some embodiments, B is an optionally substituted C₃₋₁₀ saturated carbocycle. In some embodiments, B is an optionally substituted C₃₋₁₀ unsaturated carbocycle. In some embodiments, B is selected from an optionally substituted C₃₋₆ carbocycle and optionally substituted C₆₋₁₀ carbocycle. In some embodiments, B is selected from an optionally substituted C₃₋₈ monocyclic carbocycle and optionally substituted C₆₋₁₀ bicyclic carbocycle.

In some embodiments, for the compound or salt of Formula (I), (II), (II-a), or (II-b), B is C₆₋₁₀ carbocycle optionally substituted with one or more substituents independently selected from: halogen, —OR¹², N(R¹²)₂, —C(O)R¹², —NO₂, ═O, ═S, ═N(R¹²), —CN; and C₁₋₆ alkyl optionally substituted with one or more substituents independently selected from halogen, —OR¹², N(R¹²)₂, —C(O)R¹², —NO₂, ═O, ═S, ═N(R¹²), and —CN. In some embodiments, B is C₆₋₁₀ carbocycle optionally substituted with one or more substituents independently selected from: halogen, —OR¹², N(R¹²)₂, —C(O)R¹², —NO₂, ═O, ═S, ═N(R¹²), and —CN. In some embodiments, B is C₆₋₁₀ carbocycle optionally substituted with one or more C₁₋₆ alkyl optionally substituted with one or more substituents independently selected from halogen, —OR¹², N(R¹²)₂, —C(O)R¹², —NO₂, ═O, ═S, ═N(R¹²), and —CN.

In some embodiments, for the compound or salt of Formula (I), (II), (II-a), or (II-b), B is selected from cyclohexyl and cycloheptyl, each of which is optionally substituted with one or more substituents independently selected from: halogen, —OR¹², N(R¹²)₂, —NO₂, —CN, and C₁₋₆ alkyl optionally substituted with one or more substituents independently selected from halogen, —OR¹², N(R¹²)₂, —C(O)R¹², —NO₂, ═O, ═S, ═N(R¹²), and —CN. In some embodiments, B is selected from cyclohexyl and cycloheptyl, each of which is optionally substituted with one or more substituents independently selected from halogen C₁₋₆alkyl, and C₁₋₆haloalkyl. In some embodiments, B is selected from:

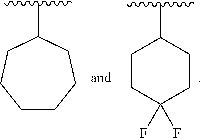 [SMILES: *C1CCCCCC1 ; *C1CCC(F)(F)CC1]

In some embodiments, B is selected from

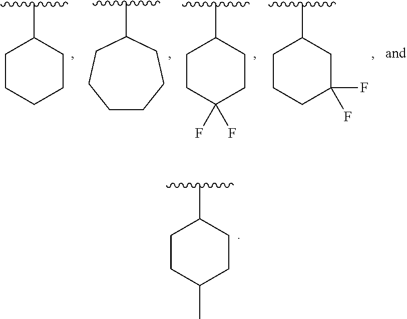

In some embodiments, B is

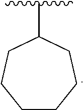 [SMILES: *C1CCCCCC1]

In some embodiments, B is

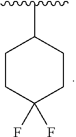 [SMILES: *C1CCC(F)(F)CC1]

In some embodiments, B is

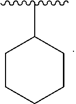 [SMILES: *C1CCCCC1]

In some embodiments, B is

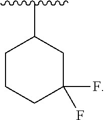 [SMILES: *C1CCCC(F)(F)C1]

In some embodiments, B is

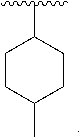 [SMILES: *C1CCC(C)CC1]

In some embodiments, for the compound or salt of Formula (I), (II), (II-a), or (II-b), B is selected from monocyclic C₃₋₇ carbocycle and bicyclic C₅₋₁₀ carbocycle, each of which is optionally substituted with one or more substituents independently selected from halogen, —OR¹², N(R¹²)₂, —C(O)R¹², —NO₂, ═O, ═S, ═N(R¹²), —CN; and C₁₋₆ alkyl and C₃₋₁₀ carbocycle, each of which is optionally substituted with one or more substituents independently selected from halogen, —OR¹², N(R¹²)₂, —C(O)R¹², —NO₂, ═O, ═S, ═N(R¹²), and —CN. In some embodiments, B is selected from monocyclic C₃₋₇ carbocycle optionally substituted with one or more substituents independently selected from halogen, —OR¹², N(R¹²)₂, —C(O)R¹², —NO₂, ═O, ═S, N(R¹²), —CN, C₁₋₆ alkyl, C₁₋₆ haloalkyl and C₃₋₆ cycloalkyl. In some embodiments, B is selected from cyclopropyl, cyclobutyl, cyclopentyl, cyclohexyl, and cycloheptyl, any of which is optionally substituted with one or more substituents independently selected from halogen, C₁₋₆ alkyl, C₁₋₆ haloalkyl and C₃₋₆ cycloalkyl. In some embodiments, B is selected from

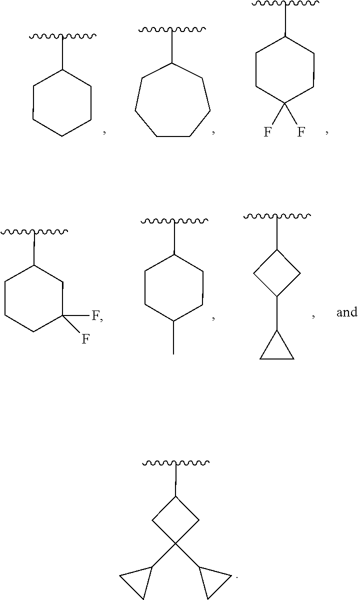

In some embodiments, for the compound or salt of Formula (I), (II), (II-a), or (II-b), B is selected from optionally substituted bicyclic C₅₋₁₀ carbocycle. In some embodiments, B is selected from optionally substituted fused C₅₋₁₀ carbocycle, optionally substituted bridged C₅₋₁₀ carbocycle, and optionally substituted spirocyclic C₅₋₁₀ carbocycle. In some embodiments, B is optionally substituted fused C₅₋₁₀ carbocycle. In some embodiments, B is optionally substituted bridged C₅₋₁₀ carbocycle. In some embodiments, B is optionally substituted spirocyclic C₅₋₁₀ carbocycle. In some embodiments, the optional substituents of the fused C₅₋₁₀ carbocycle, bridged C₅₋₁₀ carbocycle, and spirocyclic C₅₋₁₀ carbocycle are as defined as herein. In some embodiments, the optional substituents of the fused C₅₋₁₀ carbocycle, bridged C₅₋₁₀ carbocycle, and spirocyclic C₅₋₁₀ carbocycle are as defined as in Formula (I).

In some embodiments, for the compound or salt of Formula (I), (II), (II-a), or (II-b), B is selected from bicyclic C₅₋₁₀ carbocycle optionally substituted with one or more substituents independently selected from halogen, —OR¹², N(R¹²)₂, —C(O)R¹², —NO₂, ═O, ═S, N(R¹²), —CN, C₁₋₆ alkyl, and C₁₋₆ haloalkyl. In some embodiments, the bicyclic C₅₋₁₀ carbocycle of B is selected from fused C₅₋₁₀ carbocycle, bridged C₅₋₁₀ carbocycle, and spirocyclic C₅₋₁₀ carbocycle, any of which is optionally substituted with one or more substituents independently selected from halogen, C₁₋₆alkyl, and C₁₋₆ haloalkyl. In some embodiments, the bicyclic C₅₋₁₀ carbocycle of B is selected from fused C₅₋₁₀ carbocycle optionally substituted with one or more substituents independently selected from halogen, C₁₋₆alkyl, and C₁₋₆haloalkyl. In some embodiments, B is selected from

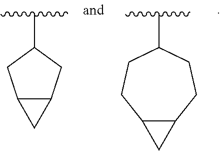 [SMILES: *C1CC2CC2C1 ; *C1CCC2CC2CC1]

In some embodiments, the bicyclic C₅₋₁₀ carbocycle of B is selected from bridged C₅₋₁₀ carbocycle optionally substituted with one or more substituents independently selected from halogen, C₁₋₆alkyl, and C₁₋₆haloalkyl. In some embodiments, B is

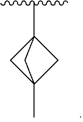 [SMILES: *C12CC(C)(C1)C2]

In some embodiments, the bicyclic C₅₋₁₀ carbocycle of B is selected from spirocyclic C₅₋₁₀ carbocycle optionally substituted with one or more substituents independently selected from halogen, C₁₋₆ alkyl, C₁₋₆ haloalkyl. In some embodiments, B is selected from

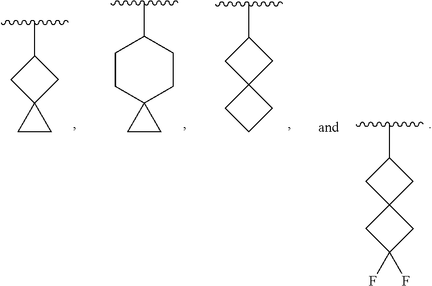 [SMILES: *C1CC2(CC2)C1 ; *C1CCC2(CC1)CC2 ; *C1CC2(CCC2)C1 ; *C1CC2(C1)CC(F)(F)C2]

In some embodiments, for the compound or salt of Formula (I), (II), (II-a), or (II-b), R¹¹, R¹², R¹³, R¹⁴, R¹⁵, R¹⁶, and R¹⁷ are each independently selected at each occurrence from:
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₆ carbocycle and 3- to 6-membered heterocycle, wherein each C₃₋₆ carbocycle and 3- to 6-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
C₃₋₆ carbocycle and 3- to 6-membered heterocycle optionally substituted with one or more substituents independently selected from: halogen, —OH, —O—C₁₋₆ alkyl, —O—C₁₋₆ haloalkyl, —NH₂, —NO₂, ═O, and —CN.

In certain aspects, the present disclosure provides a compound or salt represented by the structure of Formula (I) wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)OR¹¹, —NO₂, —CN;
   C₁₋₁₀ alkyl optionally substituted with one or more substituents independently selected halogen, —OR¹¹, —SR¹¹, —N(R¹¹)₂, —C(O)R¹¹, —C(O)OR¹¹, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —SR¹¹, —N(R¹¹)₂, —C(O)R¹¹, —C(O)OR¹¹, —NO₂, and —CN; and
   C₃₋₁₀ carbocycle optionally substituted with one or more substituents independently selected from halogen, —OR^{11A}, —SR^{11A}, —N(R^{11A})₂, —C(O)R^{11A}, —C(O)OR^{11A}, —NO₂, —CN; and C₁₋₁₀ alkyl optionally substituted with one or more substituents selected from: halogen, —OR^{11A}, —SR^{11A}, —N(R^{11A})₂, —C(O)R^{11A}, —C(O)OR^{11A}, —NO₂, and —CN, for example, A is 5-membered heteroaryl optionally substituted with C₃₋₁₀ carbocycle;
B is selected from —C(H)(R⁵)₂ and C₃₋₁₀ carbocycle, wherein each C₃₋₁₀ carbocycle is optionally substituted with one or more substituents independently selected from:
   halogen, —OR¹², —SR¹², —N(R¹²)₂, —C(O)R¹², —C(O)OR¹², —NO₂, —CN;
   C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)OR¹², —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —SR¹², —N(R¹²)₂, —C(O)R¹², —C(O)OR¹², —NO₂, and —CN, for example B is optionally substituted C₃₋₁₀ carbocycle;
each R⁵ is independently selected at each occurrence from (i), (ii), and (iii):
   (i) halogen, —OR¹³, —SR¹³, —N(R¹³)₂, —C(O)R¹³, —C(O)OR¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN; and
   (iii) C₃₋₁₀ carbocycle optionally substituted with one or more substituents independently selected from: halogen, —OR¹³, —SR¹³, —N(R¹³)₂, —C(O)R¹³, —C(O)OR¹³, —NO₂, —CN, and C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN,
   for example, each R⁵ is optionally substituted C₃₋₁₀ carbocycle or optionally substituted C₁₋₁₀ alkyl;
each R¹ is independently selected at each occurrence from:
   halogen, —OR¹⁴, —SR¹⁴, —N(R¹⁴)₂, —C(O)R¹⁴, —C(O)OR¹⁴, —NO₂, —CN;
   C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN; and
   C₃₋₁₀ carbocycle optionally substituted with one or more substituents independently selected from halogen, —OR¹⁴, —SR¹⁴, —N(R¹⁴)₂, —C(O)R¹⁴, —C(O)OR¹⁴, —NO₂, —CN; and C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN, for example, each R¹ is C₁₋₁₀ haloalkyl;
R² is selected from:
   hydrogen, halogen, —OR¹⁵, —SR¹⁵, —N(R¹⁵)₂, —C(O)R¹⁵, —C(O)OR¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)OR¹⁵, —NO₂, and —CN, for example R² is hydrogen;
each R³ is independently selected at each occurrence from:
   hydrogen, halogen, —OR¹⁶, —SR¹⁶, —N(R¹⁶)₂, —C(O)R¹⁶, —C(O)OR¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)OR¹⁶, —NO₂, and —CN, for example each R³ hydrogen or optionally substituted C₁₋₆ alkyl;
each R⁴ is independently selected at each occurrence from
   halogen, —OR¹⁷, —SR¹⁷, —N(R¹⁷)₂, —C(O)R¹⁷, —C(O)OR¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)OR¹⁷, —NO₂, and —CN, for example each R⁴ is halogen or optionally substituted C₁₋₁₀ alkyl;
R¹¹, R^{11A}, R¹², R¹³, R¹⁴, R¹⁵, R¹⁶, and R¹⁷ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2, for example p is 1;
m is selected from 0 and 1, for example m is 0; and
p is selected from 0, 1, and 2, for example p is 1.

In certain aspects, the present disclosure provides a compound or salt represented by the structure of Formula (I) wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)OR¹¹, —NO₂, —CN;
   C₁₋₁₀ alkyl optionally substituted with one or more substituents independently selected halogen, —OR¹¹, —SR¹¹, —N(R¹¹)₂, —C(O)R¹¹, —C(O)OR¹¹, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —SR¹¹, —N(R¹¹)₂, —C(O)R¹¹, —C(O)OR¹¹, —NO₂, and —CN; and
   C₃₋₁₀ carbocycle optionally substituted with one or more substituents independently selected from halogen, —OR^{11A}, —SR^{11A}, —N(R^{11A})₂, —C(O)R^{11A}, —C(O)OR^{11A}, —NO₂, —CN; and C₁₋₁₀ alkyl optionally substituted with one or more substituents selected from: halogen, —OR^{11A}, —SR^{11A}, —N(R^{11A})₂, —C(O)R^{11A}, —C(O)OR^{11A}, —NO₂, and —CN, for example A is 5-membered heteroaryl optionally substituted with C₃₋₁₀ carbocycle;
B is —C(H)(R⁵)₂ and each R⁵ is independently selected at each occurrence from (i), (ii), and (iii):
   (i) halogen, —OR¹³, —SR¹³, —N(R¹³)₂, —C(O)R¹³, —C(O)OR¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN; and
   (iii) C₃₋₁₀ carbocycle optionally substituted with one or more substituents independently selected from: halogen, —OR¹³, —SR¹³, —N(R¹³)₂, —C(O)R¹³, —C(O)OR¹³, —NO₂, —CN
      C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN,
   for example, each R⁵ is selected from optionally substituted C₃₋₁₀ carbocycle or optionally substituted C₁₋₁₀ alkyl;
each R¹ is independently selected at each occurrence from:
   halogen, —OR¹⁴, —SR¹⁴, —N(R¹⁴)₂, —C(O)R¹⁴, —C(O)OR¹⁴, —NO₂, —CN;
   C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN; and
   C₃₋₁₀ carbocycle optionally substituted with one or more substituents independently selected from halogen, —OR¹⁴, —SR¹⁴, —N(R¹⁴)₂, —C(O)R¹⁴, —C(O)OR¹⁴, —NO₂, —CN; and C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN, for example, each R¹ is C₁₋₁₀ haloalkyl;
R² is selected from:
   hydrogen, halogen, —OR¹⁵, —SR¹⁵, —N(R¹⁵)₂, —C(O)R¹⁵, —C(O)OR¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)OR¹⁵, —NO₂, and —CN, for example R² is hydrogen;
each R³ is independently selected at each occurrence from:
   hydrogen, halogen, —OR¹⁶, —SR¹⁶, —N(R¹⁶)₂, —C(O)R¹⁶, —C(O)OR¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)OR¹⁶, —NO₂, and —CN, for example each R³ hydrogen or optionally substituted C₁₋₆ alkyl;
each R⁴ is independently selected at each occurrence from
   halogen, —OR¹⁷, —SR¹⁷, —N(R¹⁷)₂, —C(O)R¹⁷, —C(O)OR¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)OR¹⁷, —NO₂, and —CN, for example each R⁴ is halogen or optionally substituted C₁₋₁₀ alkyl;
R¹¹, R^{11A}, R¹², R¹³, R¹⁴, R¹⁵, R¹⁶, and R¹⁷ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2, for example p is 1;
m is selected from 0 and 1, for example m is 0; and
p is selected from 0, 1, and 2, for example p is 1.

In certain aspects, the present disclosure provides a compound or salt represented by the structure of Formula (I), (II), (II-a), or (II-b), wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)OR¹¹, —NO₂, —CN; and
   C₁₋₁₀ alkyl optionally substituted with one or more substituents independently selected halogen, —OR¹¹, —SR¹¹, —N(R¹¹)₂, —C(O)R¹¹, —C(O)OR¹¹, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —SR¹¹, —N(R¹¹)₂, —C(O)R¹¹, —C(O)OR¹¹, —NO₂, and —CN, for example, A is 5-membered heteroaryl optionally substituted with C₁₋₁₀alkyl;
B is selected from —C(H)(R⁵)₂ and C₃₋₁₀ carbocycle, wherein each C₃₋₁₀ carbocycle is optionally substituted with one or more substituents independently selected from:
   halogen, —OR¹², —SR¹², —N(R¹²)₂, —C(O)R¹², —C(O)OR¹², —NO₂, —CN;
   C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)OR¹², —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —SR¹², —N(R¹²)₂, —C(O)R¹², —C(O)OR¹², —NO₂, and —CN, for example B is optionally substituted C₃₋₁₀ carbocycle;
each R⁵ is independently selected at each occurrence from (i), (ii), and (iii):
   (i) halogen, —OR¹³, —SR¹³, —N(R¹³)₂, —C(O)R¹³, —C(O)OR¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN; and
   (iii) C₃₋₁₀ carbocycle optionally substituted with one or more substituents independently selected from: halogen, —OR¹³, —SR¹³, —N(R¹³)₂, —C(O)R¹³, —C(O)OR¹³, —NO₂, —CN, and C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN,
   for example, each R⁵ is optionally substituted C₃₋₁₀ carbocycle or optionally substituted C₁₋₁₀ alkyl;
each R¹ is independently selected at each occurrence from:
   halogen, —OR¹⁴, —SR¹⁴, —N(R¹⁴)₂, —C(O)R¹⁴, —C(O)OR¹⁴, —NO₂, —CN;
   C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN; and
   C₃₋₁₀ carbocycle optionally substituted with one or more substituents independently selected from halogen, —OR¹⁴, —SR¹⁴, —N(R¹⁴)₂, —C(O)R¹⁴, —C(O)OR¹⁴, —NO₂, —CN; and C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN, for example, each R¹ is C₁₋₁₀ haloalkyl;
R² is selected from:
   hydrogen, halogen, —OR¹⁵, —SR¹⁵, —N(R¹⁵)₂, —C(O)R¹⁵, —C(O)OR¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)OR¹⁵, —NO₂, and —CN, for example R² is hydrogen;
each R³ is independently selected at each occurrence from:
   hydrogen, halogen, —OR¹⁶, —SR¹⁶, —N(R¹⁶)₂, —C(O)R¹⁶, —C(O)OR¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)OR¹⁶, —NO₂, and —CN, for example each R³ hydrogen or optionally substituted C₁₋₆ alkyl;
each R⁴ is independently selected at each occurrence from
   halogen, —OR¹⁷, —SR¹⁷, —N(R¹⁷)₂, —C(O)R¹⁷, —C(O)OR¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)OR¹⁷, —NO₂, and —CN, for example each R⁴ is halogen or optionally substituted C₁₋₁₀ alkyl;
R¹¹, R¹², R¹³, R¹⁴, R¹⁵, R¹⁶, and R¹⁷ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2, for example p is 1;
m is selected from 0 and 1, for example m is 0; and
p is selected from 0, 1, and 2, for example p is 1.

In certain aspects, the present disclosure provides a compound or salt represented by the structure of Formula (I), (II), (II-a), or (II-b), wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)OR¹¹, —NO₂, —CN;
   C₁₋₁₀ alkyl optionally substituted with one or more substituents independently selected halogen, —OR¹¹, —SR¹¹, —N(R¹¹)₂, —C(O)R¹¹, —C(O)OR¹¹, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —SR¹¹, —N(R¹¹)₂, —C(O)R¹¹, —C(O)OR¹¹, —NO₂, and —CN; and for example A is 5-membered heteroaryl optionally substituted with C₁₋₁₀ alkyl;
B is —C(H)(R⁵)₂ and each R⁵ is independently selected at each occurrence from (i), (ii), and (iii):
   (i) halogen, —OR¹³, —SR¹³, —N(R¹³)₂, —C(O)R¹³, —C(O)OR¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN; and
   (iii) C₃₋₁₀ carbocycle optionally substituted with one or more substituents independently selected from: halogen, —OR¹³, —SR¹³, —N(R¹³)₂, —C(O)R¹³, —C(O)OR¹³, —NO₂, —CN
      C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN,
   for example, each R⁵ is selected from optionally substituted C₃₋₁₀ carbocycle or optionally substituted C₁₋₁₀ alkyl;
each R¹ is independently selected at each occurrence from:
   halogen, —OR¹⁴, —SR¹⁴, —N(R¹⁴)₂, —C(O)R¹⁴, —C(O)OR¹⁴, —NO₂, —CN;
   C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN; and
   C₃₋₁₀ carbocycle optionally substituted with one or more substituents independently selected from halogen, —OR¹⁴, —SR¹⁴, —N(R¹⁴)₂, —C(O)R¹⁴, —C(O)OR¹⁴, —NO₂, —CN; and C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN, for example, each R¹ is C₁₋₁₀ haloalkyl;
R² is selected from:
   hydrogen, halogen, —OR¹⁵, —SR¹⁵, —N(R¹⁵)₂, —C(O)R¹⁵, —C(O)OR¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)OR¹⁵, —NO₂, and —CN, for example R² is hydrogen;
each R³ is independently selected at each occurrence from:
   hydrogen, halogen, —OR¹⁶, —SR¹⁶, —N(R¹⁶)₂, —C(O)R¹⁶, —C(O)OR¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)OR¹⁶, —NO₂, and —CN, for example each R³ hydrogen or optionally substituted C₁₋₆ alkyl;
each R⁴ is independently selected at each occurrence from
   halogen, —OR¹⁷, —SR¹⁷, —N(R¹⁷)₂, —C(O)R¹⁷, —C(O)OR¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)OR¹⁷, —NO₂, and —CN, for example each R⁴ is halogen or optionally substituted C₁₋₁₀ alkyl;
R¹¹, R¹², R¹³, R¹⁴, R¹⁵, R¹⁶, and R¹⁷ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2, for example p is 1;
m is selected from 0 and 1, for example m is 0; and
p is selected from 0, 1, and 2, for example p is 1.

In some aspects, the present disclosure provides a compound of Formula (I) selected from:

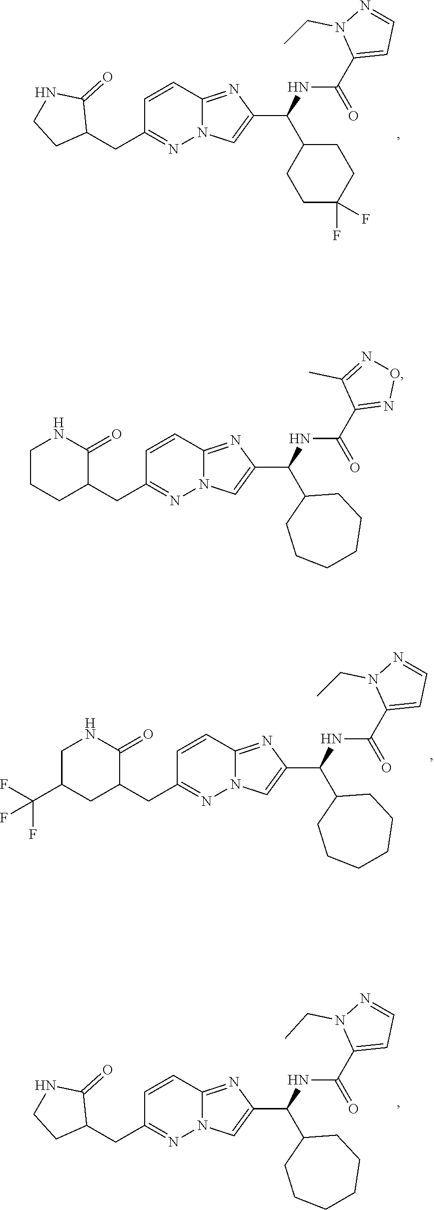 [SMILES: CCn1nccc1C(=O)N[C@H](c1cn2nc(CC3CCNC3=O)ccc2n1)C1CCC(F)(F)CC1 ; Cc1nonc1C(=O)N[C@H](c1cn2nc(CC3CCCNC3=O)ccc2n1)C1CCCCCC1 ; CCn1nccc1C(=O)N[C@H](c1cn2nc(CC3CC(C(F)(F)F)CNC3=O)ccc2n1)C1CCCCCC1 ; CCn1nccc1C(=O)N[C@H](c1cn2nc(CC3CCNC3=O)ccc2n1)C1CCCCCC1]

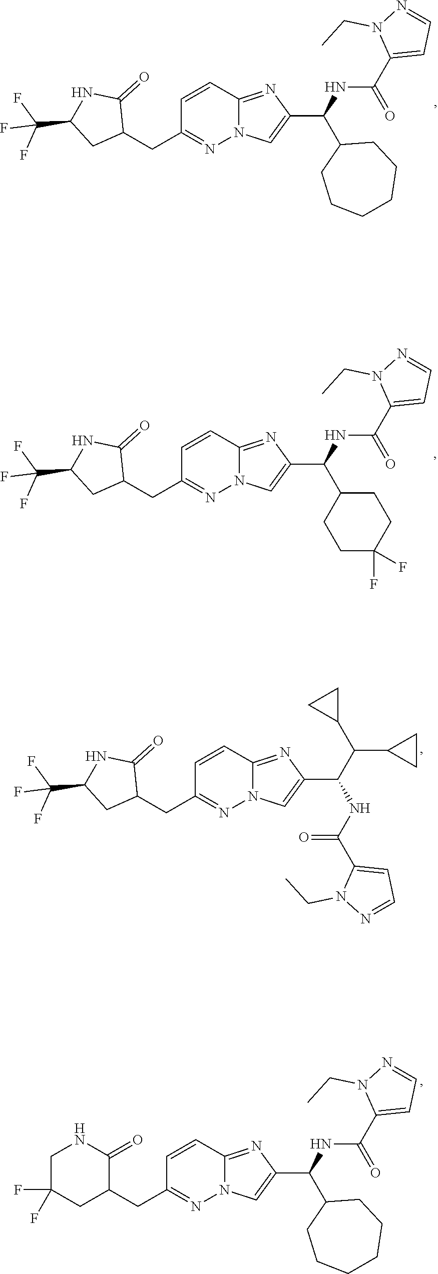 [SMILES: CCn1nccc1C(=O)N[C@H](c1cn2nc(CC3C[C@@H](C(F)(F)F)NC3=O)ccc2n1)C1CCCCCC1 ; CCn1nccc1C(=O)N[C@H](c1cn2nc(CC3C[C@@H](C(F)(F)F)NC3=O)ccc2n1)C1CCC(F)(F)CC1 ; CCn1nccc1C(=O)N[C@H](c1cn2nc(CC3C[C@@H](C(F)(F)F)NC3=O)ccc2n1)C(C1CC1)C1CC1 ; CCn1nccc1C(=O)N[C@H](c1cn2nc(CC3CC(F)(F)CNC3=O)ccc2n1)C1CCCCCC1]

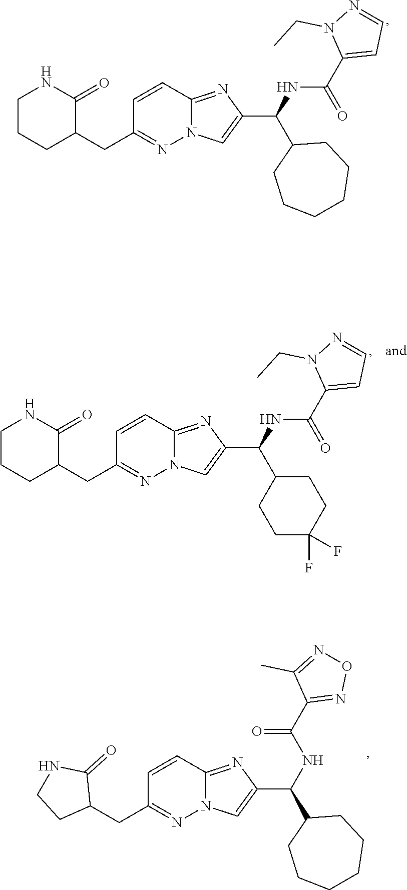 [SMILES: CCn1nccc1C(=O)N[C@H](c1cn2nc(CC3CCCNC3=O)ccc2n1)C1CCCCCC1 ; CCn1nccc1C(=O)N[C@H](c1cn2nc(CC3CCCNC3=O)ccc2n1)C1CCC(F)(F)CC1 ; Cc1nonc1C(=O)N[C@H](c1cn2nc(CC3CCNC3=O)ccc2n1)C1CCCCCC1]

or pharmaceutically acceptable salts thereof.

In some aspects, the present disclosure provides a compound of Formula (I) selected from:

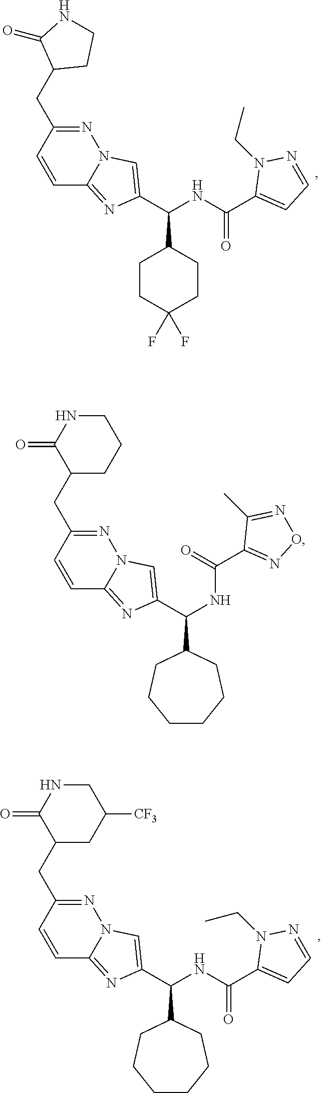 [SMILES: CCn1nccc1C(=O)N[C@H](c1cn2nc(CC3CCNC3=O)ccc2n1)C1CCC(F)(F)CC1 ; Cc1nonc1C(=O)N[C@H](c1cn2nc(CC3CCCNC3=O)ccc2n1)C1CCCCCC1 ; CCn1nccc1C(=O)N[C@H](c1cn2nc(CC3CC(C(F)(F)F)CNC3=O)ccc2n1)C1CCCCCC1]

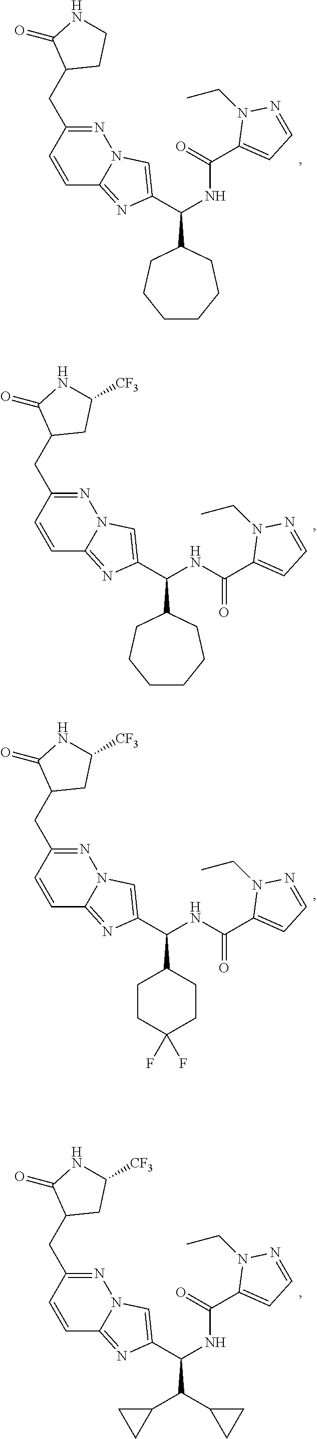 [SMILES: CCn1nccc1C(=O)N[C@H](c1cn2nc(CC3CCNC3=O)ccc2n1)C1CCCCCC1 ; CCn1nccc1C(=O)N[C@H](c1cn2nc(CC3C[C@@H](C(F)(F)F)NC3=O)ccc2n1)C1CCCCCC1 ; CCn1nccc1C(=O)N[C@H](c1cn2nc(CC3C[C@@H](C(F)(F)F)NC3=O)ccc2n1)C1CCC(F)(F)CC1 ; CCn1nccc1C(=O)N[C@H](c1cn2nc(CC3C[C@@H](C(F)(F)F)NC3=O)ccc2n1)C(C1CC1)C1CC1]

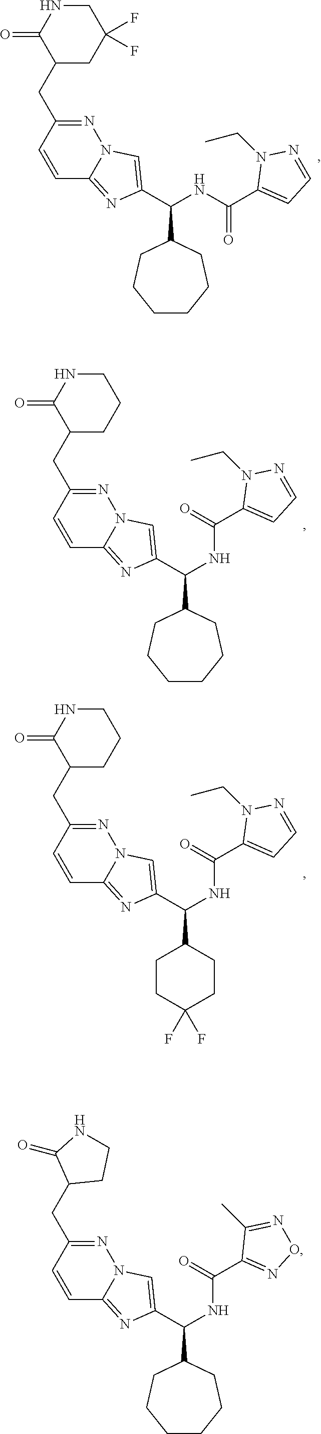 [SMILES: CCn1nccc1C(=O)N[C@H](c1cn2nc(CC3CC(F)(F)CNC3=O)ccc2n1)C1CCCCCC1 ; CCn1nccc1C(=O)N[C@H](c1cn2nc(CC3CCCNC3=O)ccc2n1)C1CCCCCC1 ; CCn1nccc1C(=O)N[C@H](c1cn2nc(CC3CCCNC3=O)ccc2n1)C1CCC(F)(F)CC1 ; Cc1nonc1C(=O)N[C@H](c1cn2nc(CC3CCNC3=O)ccc2n1)C1CCCCCC1]

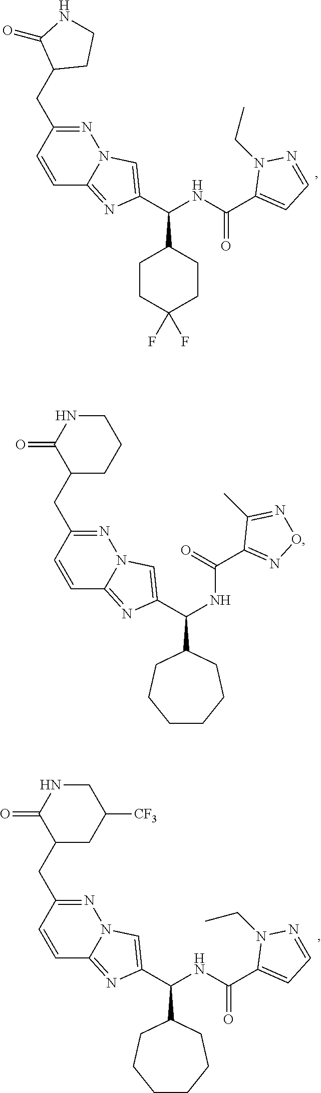 [SMILES: CCn1nccc1C(=O)N[C@H](c1cn2nc(CC3CCNC3=O)ccc2n1)C1CCC(F)(F)CC1 ; Cc1nonc1C(=O)N[C@H](c1cn2nc(CC3CCCNC3=O)ccc2n1)C1CCCCCC1 ; CCn1nccc1C(=O)N[C@H](c1cn2nc(CC3CC(C(F)(F)F)CNC3=O)ccc2n1)C1CCCCCC1]

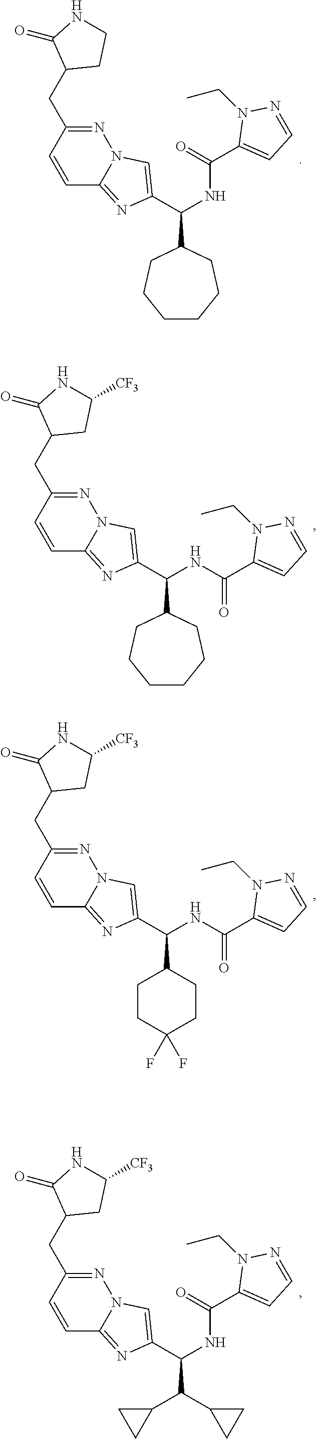 [SMILES: CCn1nccc1C(=O)N[C@H](c1cn2nc(CC3CCNC3=O)ccc2n1)C1CCCCCC1 ; CCn1nccc1C(=O)N[C@H](c1cn2nc(CC3C[C@@H](C(F)(F)F)NC3=O)ccc2n1)C1CCCCCC1 ; CCn1nccc1C(=O)N[C@H](c1cn2nc(CC3C[C@@H](C(F)(F)F)NC3=O)ccc2n1)C1CCC(F)(F)CC1 ; CCn1nccc1C(=O)N[C@H](c1cn2nc(CC3C[C@@H](C(F)(F)F)NC3=O)ccc2n1)C(C1CC1)C1CC1]

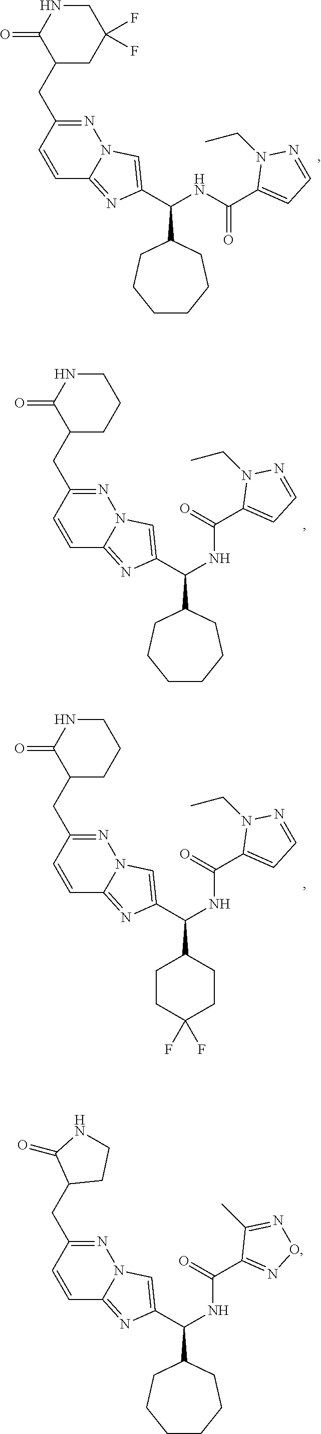 [SMILES: CCn1nccc1C(=O)N[C@H](c1cn2nc(CC3CC(F)(F)CNC3=O)ccc2n1)C1CCCCCC1 ; CCn1nccc1C(=O)N[C@H](c1cn2nc(CC3CCCNC3=O)ccc2n1)C1CCCCCC1 ; CCn1nccc1C(=O)N[C@H](c1cn2nc(CC3CCCNC3=O)ccc2n1)C1CCC(F)(F)CC1 ; Cc1nonc1C(=O)N[C@H](c1cn2nc(CC3CCNC3=O)ccc2n1)C1CCCCCC1]

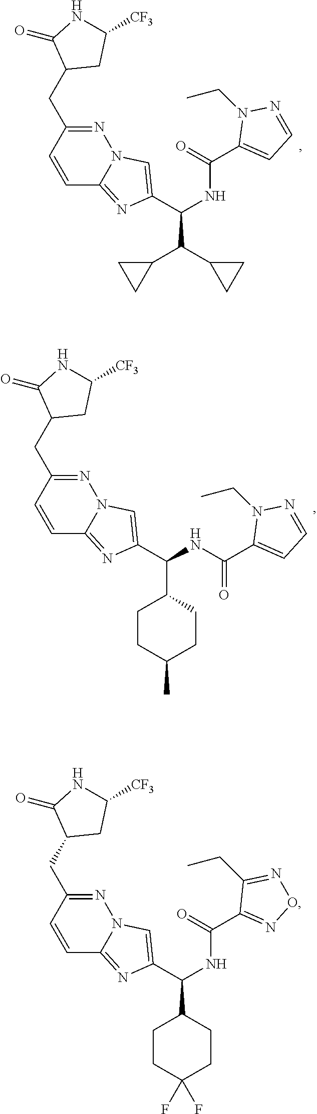 [SMILES: CCn1nccc1C(=O)N[C@H](c1cn2nc(CC3C[C@@H](C(F)(F)F)NC3=O)ccc2n1)C(C1CC1)C1CC1 ; CCn1nccc1C(=O)N[C@H](c1cn2nc(CC3C[C@@H](C(F)(F)F)NC3=O)ccc2n1)[C@H]1CC[C@H](C)CC1 ; CCc1nonc1C(=O)N[C@H](c1cn2nc(C[C@H]3C[C@@H](C(F)(F)F)NC3=O)ccc2n1)C1CCC(F)(F)CC1]

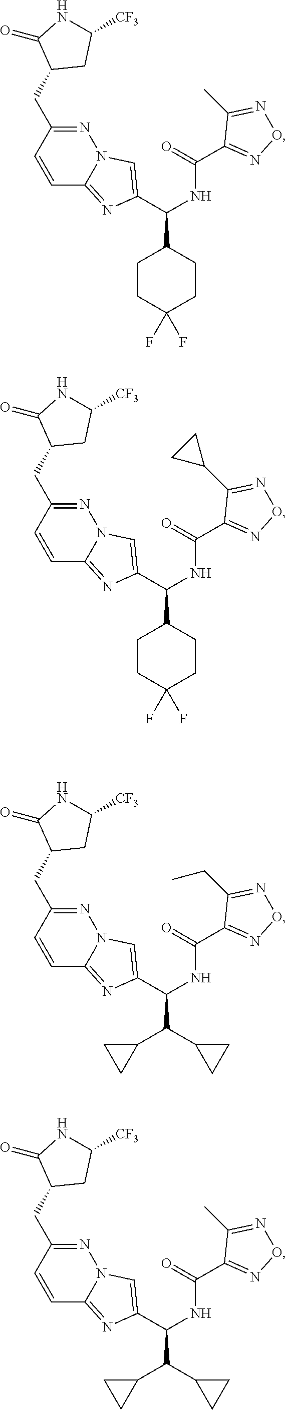 [SMILES: Cc1nonc1C(=O)N[C@H](c1cn2nc(C[C@H]3C[C@@H](C(F)(F)F)NC3=O)ccc2n1)C1CCC(F)(F)CC1 ; O=C(N[C@H](c1cn2nc(C[C@H]3C[C@@H](C(F)(F)F)NC3=O)ccc2n1)C1CCC(F)(F)CC1)c1nonc1C1CC1 ; CCc1nonc1C(=O)N[C@H](c1cn2nc(C[C@H]3C[C@@H](C(F)(F)F)NC3=O)ccc2n1)C(C1CC1)C1CC1 ; Cc1nonc1C(=O)N[C@H](c1cn2nc(C[C@H]3C[C@@H](C(F)(F)F)NC3=O)ccc2n1)C(C1CC1)C1CC1]

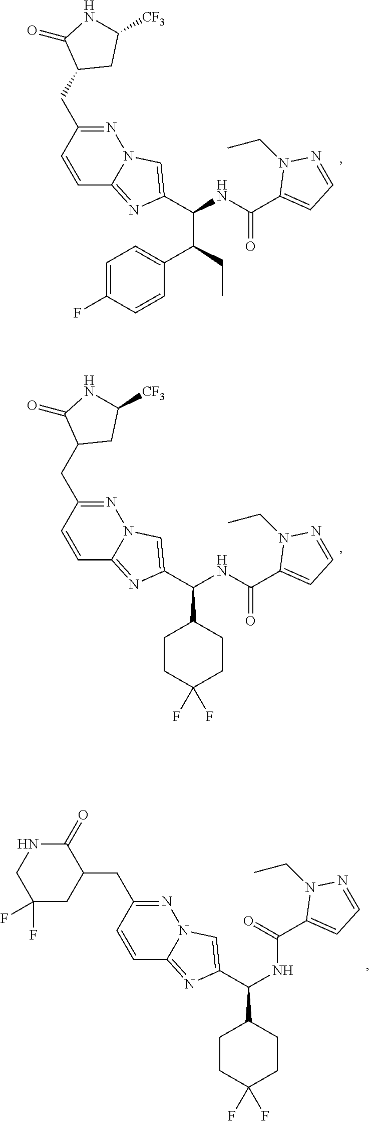 [SMILES: CC[C@@H](c1ccc(F)cc1)[C@H](NC(=O)c1ccnn1CC)c1cn2nc(C[C@H]3C[C@@H](C(F)(F)F)NC3=O)ccc2n1 ; CCn1nccc1C(=O)N[C@H](c1cn2nc(CC3C[C@H](C(F)(F)F)NC3=O)ccc2n1)C1CCC(F)(F)CC1 ; CCn1nccc1C(=O)N[C@H](c1cn2nc(CC3CC(F)(F)CNC3=O)ccc2n1)C1CCC(F)(F)CC1]

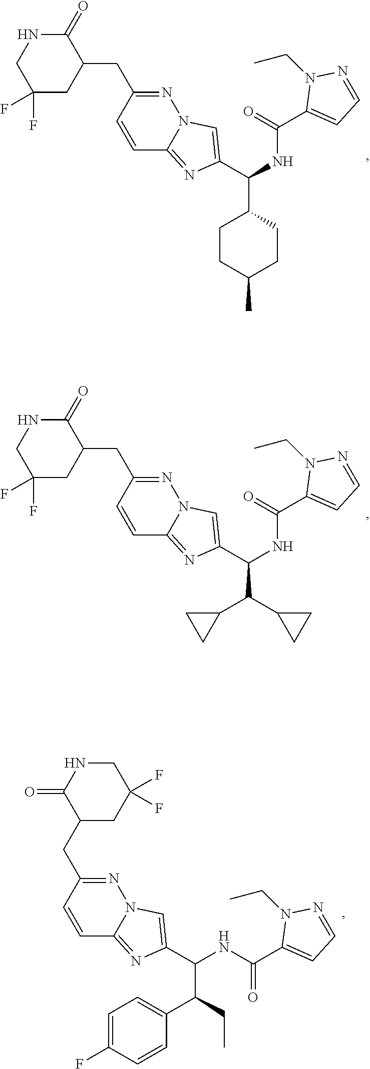 [SMILES: CCn1nccc1C(=O)N[C@H](c1cn2nc(CC3CC(F)(F)CNC3=O)ccc2n1)[C@H]1CC[C@H](C)CC1 ; CCn1nccc1C(=O)N[C@H](c1cn2nc(CC3CC(F)(F)CNC3=O)ccc2n1)C(C1CC1)C1CC1 ; CC[C@@H](c1ccc(F)cc1)C(NC(=O)c1ccnn1CC)c1cn2nc(CC3CC(F)(F)CNC3=O)ccc2n1]

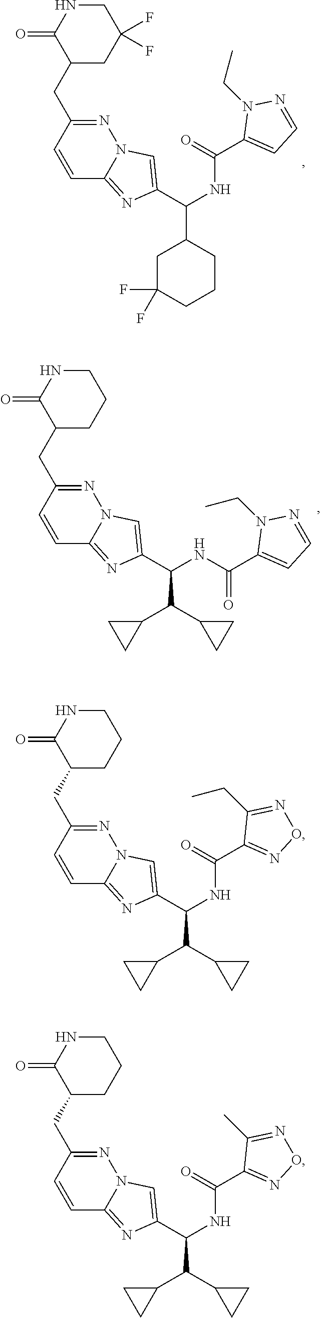 [SMILES: CCn1nccc1C(=O)NC(c1cn2nc(CC3CC(F)(F)CNC3=O)ccc2n1)C1CCCC(F)(F)C1 ; CCn1nccc1C(=O)N[C@H](c1cn2nc(CC3CCCNC3=O)ccc2n1)C(C1CC1)C1CC1 ; CCc1nonc1C(=O)N[C@H](c1cn2nc(C[C@H]3CCCNC3=O)ccc2n1)C(C1CC1)C1CC1 ; Cc1nonc1C(=O)N[C@H](c1cn2nc(C[C@H]3CCCNC3=O)ccc2n1)C(C1CC1)C1CC1]

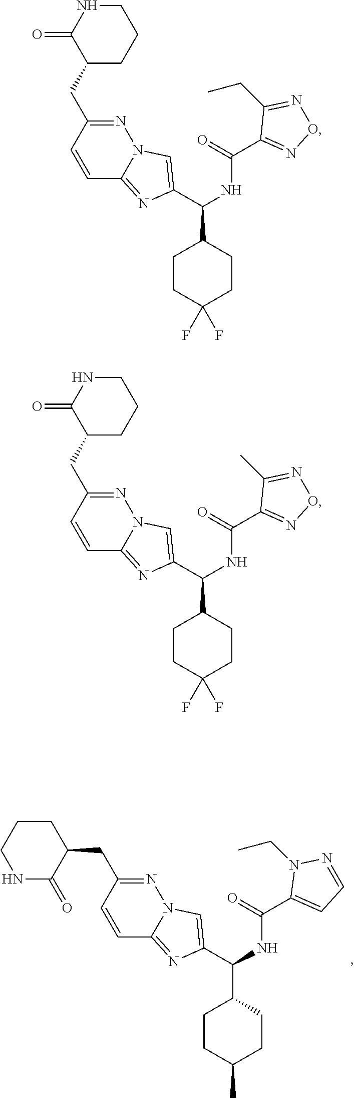 [SMILES: CCc1nonc1C(=O)N[C@H](c1cn2nc(C[C@H]3CCCNC3=O)ccc2n1)C1CCC(F)(F)CC1 ; Cc1nonc1C(=O)N[C@H](c1cn2nc(C[C@H]3CCCNC3=O)ccc2n1)C1CCC(F)(F)CC1 ; CCn1nccc1C(=O)N[C@H](c1cn2nc(C[C@@H]3CCCNC3=O)ccc2n1)[C@H]1CC[C@H](C)CC1]

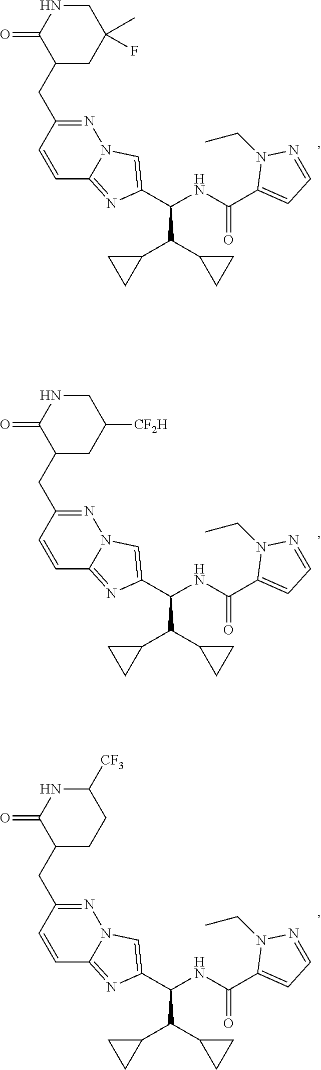 [SMILES: CCn1nccc1C(=O)N[C@H](c1cn2nc(CC3CC(C)(F)CNC3=O)ccc2n1)C(C1CC1)C1CC1 ; CCn1nccc1C(=O)N[C@H](c1cn2nc(CC3CC(C(F)F)CNC3=O)ccc2n1)C(C1CC1)C1CC1 ; CCn1nccc1C(=O)N[C@H](c1cn2nc(CC3CCC(C(F)(F)F)NC3=O)ccc2n1)C(C1CC1)C1CC1]

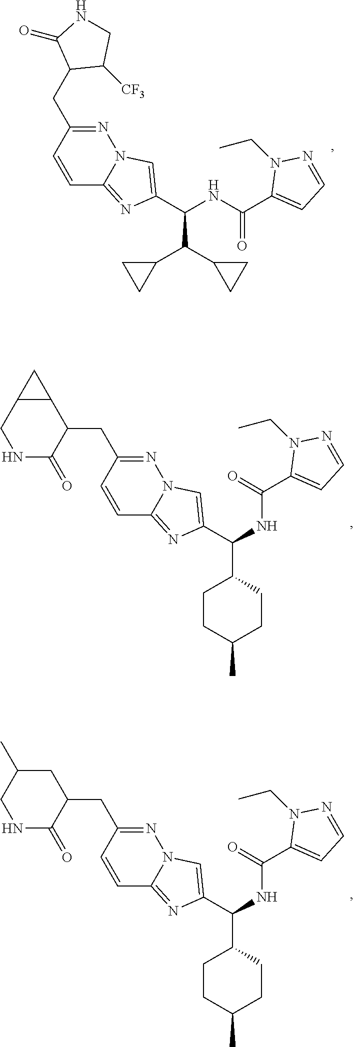 [SMILES: CCn1nccc1C(=O)N[C@H](c1cn2nc(CC3C(=O)NCC3C(F)(F)F)ccc2n1)C(C1CC1)C1CC1 ; CCn1nccc1C(=O)N[C@H](c1cn2nc(CC3C(=O)NCC4CC43)ccc2n1)[C@H]1CC[C@H](C)CC1 ; CCn1nccc1C(=O)N[C@H](c1cn2nc(CC3CC(C)CNC3=O)ccc2n1)[C@H]1CC[C@H](C)CC1]

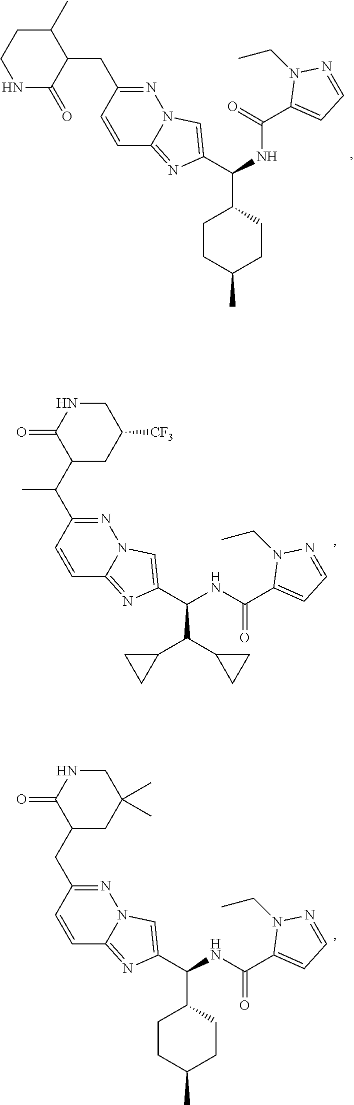 [SMILES: CCn1nccc1C(=O)N[C@H](c1cn2nc(CC3C(=O)NCCC3C)ccc2n1)[C@H]1CC[C@H](C)CC1 ; CCn1nccc1C(=O)N[C@H](c1cn2nc(C(C)C3C[C@@H](C(F)(F)F)CNC3=O)ccc2n1)C(C1CC1)C1CC1 ; CCn1nccc1C(=O)N[C@H](c1cn2nc(CC3CC(C)(C)CNC3=O)ccc2n1)[C@H]1CC[C@H](C)CC1]

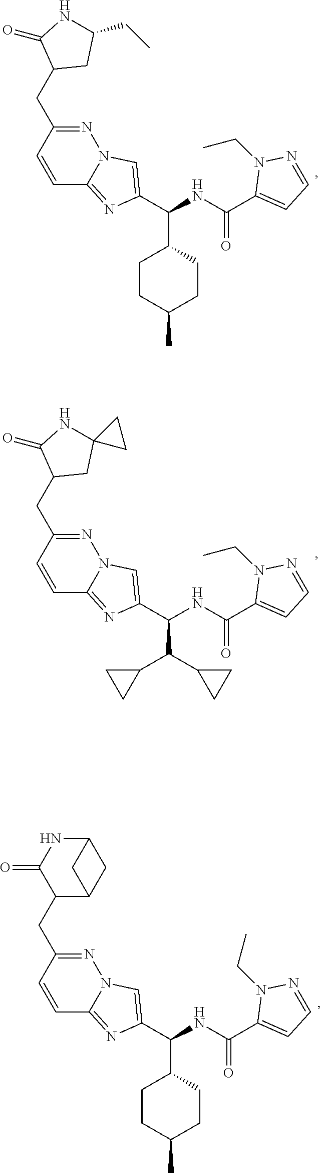 [SMILES: CC[C@@H]1CC(Cc2ccc3nc([C@@H](NC(=O)c4ccnn4CC)[C@H]4CC[C@H](C)CC4)cn3n2)C(=O)N1 ; CCn1nccc1C(=O)N[C@H](c1cn2nc(CC3CC4(CC4)NC3=O)ccc2n1)C(C1CC1)C1CC1 ; CCn1nccc1C(=O)N[C@H](c1cn2nc(CC3C(=O)NC4CC3C4)ccc2n1)[C@H]1CC[C@H](C)CC1]

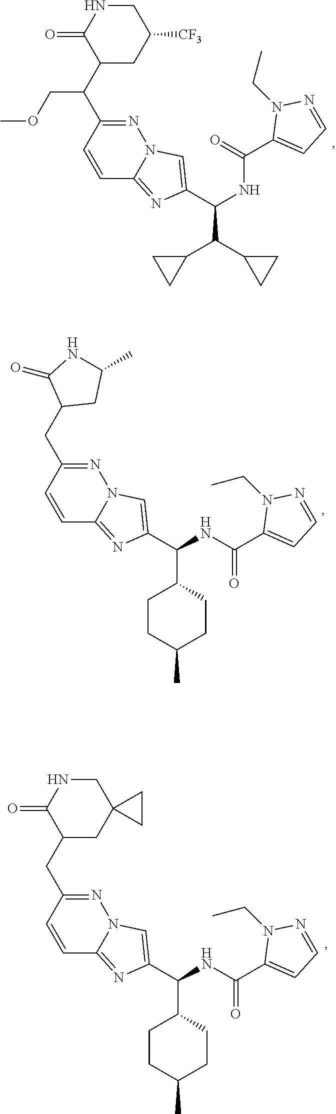 [SMILES: CCn1nccc1C(=O)N[C@H](c1cn2nc(C(COC)C3C[C@@H](C(F)(F)F)CNC3=O)ccc2n1)C(C1CC1)C1CC1 ; CCn1nccc1C(=O)N[C@H](c1cn2nc(CC3C[C@@H](C)NC3=O)ccc2n1)[C@H]1CC[C@H](C)CC1 ; CCn1nccc1C(=O)N[C@H](c1cn2nc(CC3CC4(CC4)CNC3=O)ccc2n1)[C@H]1CC[C@H](C)CC1]

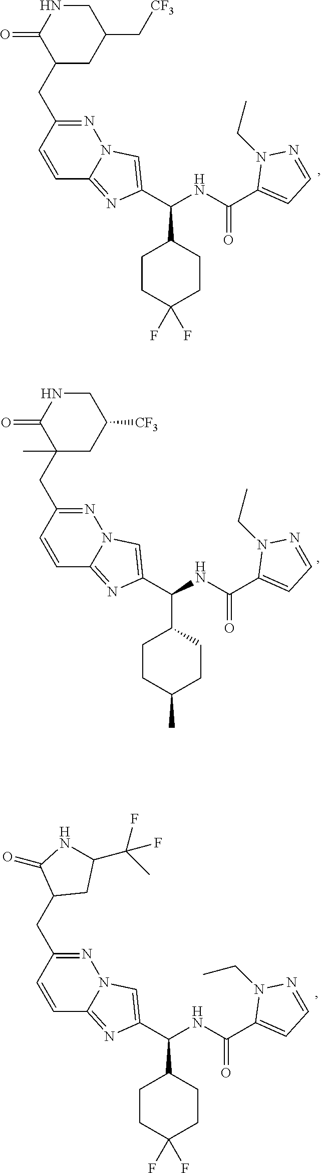 [SMILES: CCn1nccc1C(=O)N[C@H](c1cn2nc(CC3CC(CC(F)(F)F)CNC3=O)ccc2n1)C1CCC(F)(F)CC1 ; CCn1nccc1C(=O)N[C@H](c1cn2nc(CC3(C)C[C@@H](C(F)(F)F)CNC3=O)ccc2n1)[C@H]1CC[C@H](C)CC1 ; CCn1nccc1C(=O)N[C@H](c1cn2nc(CC3CC(C(C)(F)F)NC3=O)ccc2n1)C1CCC(F)(F)CC1]

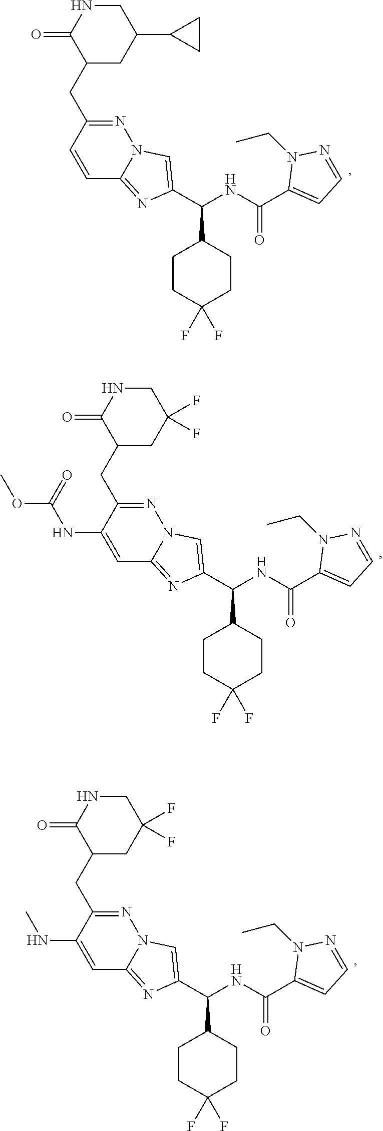 [SMILES: CCn1nccc1C(=O)N[C@H](c1cn2nc(CC3CC(C4CC4)CNC3=O)ccc2n1)C1CCC(F)(F)CC1 ; CCn1nccc1C(=O)N[C@H](c1cn2nc(CC3CC(F)(F)CNC3=O)c(NC(=O)OC)cc2n1)C1CCC(F)(F)CC1 ; CCn1nccc1C(=O)N[C@H](c1cn2nc(CC3CC(F)(F)CNC3=O)c(NC)cc2n1)C1CCC(F)(F)CC1]

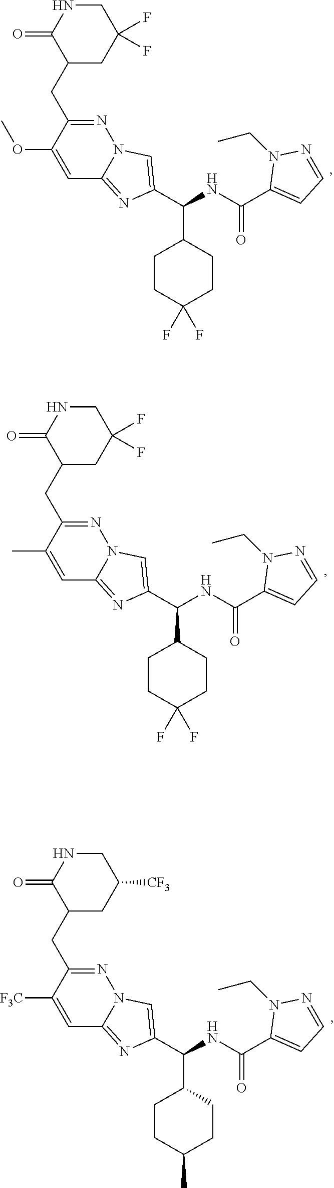 [SMILES: CCn1nccc1C(=O)N[C@H](c1cn2nc(CC3CC(F)(F)CNC3=O)c(OC)cc2n1)C1CCC(F)(F)CC1 ; CCn1nccc1C(=O)N[C@H](c1cn2nc(CC3CC(F)(F)CNC3=O)c(C)cc2n1)C1CCC(F)(F)CC1 ; CCn1nccc1C(=O)N[C@H](c1cn2nc(CC3C[C@@H](C(F)(F)F)CNC3=O)c(C(F)(F)F)cc2n1)[C@H]1CC[C@H](C)CC1]

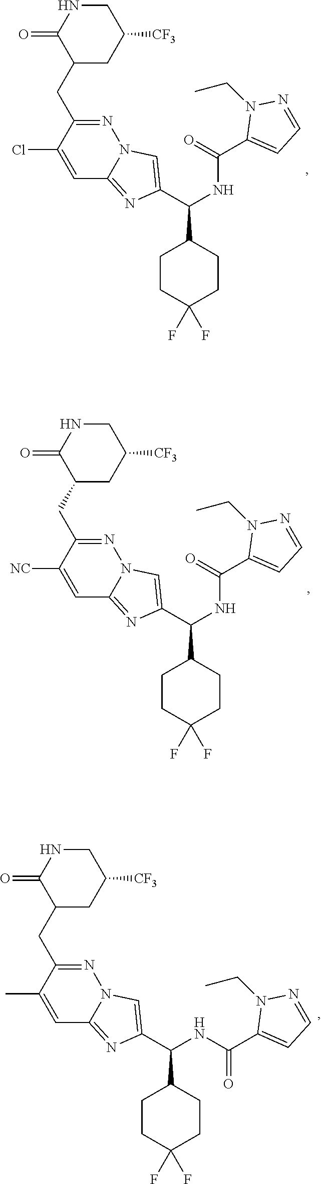 [SMILES: CCn1nccc1C(=O)N[C@H](c1cn2nc(CC3C[C@@H](C(F)(F)F)CNC3=O)c(Cl)cc2n1)C1CCC(F)(F)CC1 ; CCn1nccc1C(=O)N[C@H](c1cn2nc(C[C@H]3C[C@@H](C(F)(F)F)CNC3=O)c(C#N)cc2n1)C1CCC(F)(F)CC1 ; CCn1nccc1C(=O)N[C@H](c1cn2nc(CC3C[C@@H](C(F)(F)F)CNC3=O)c(C)cc2n1)C1CCC(F)(F)CC1]

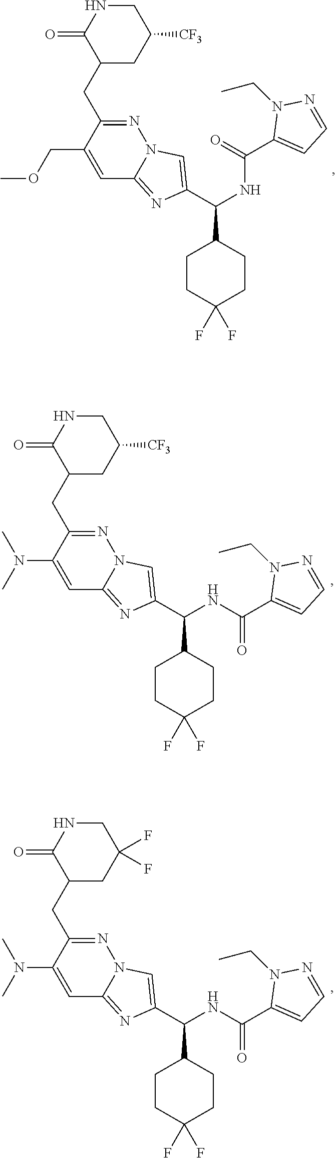 [SMILES: CCn1nccc1C(=O)N[C@H](c1cn2nc(CC3C[C@@H](C(F)(F)F)CNC3=O)c(COC)cc2n1)C1CCC(F)(F)CC1 ; CCn1nccc1C(=O)N[C@H](c1cn2nc(CC3C[C@@H](C(F)(F)F)CNC3=O)c(N(C)C)cc2n1)C1CCC(F)(F)CC1 ; CCn1nccc1C(=O)N[C@H](c1cn2nc(CC3CC(F)(F)CNC3=O)c(N(C)C)cc2n1)C1CCC(F)(F)CC1]

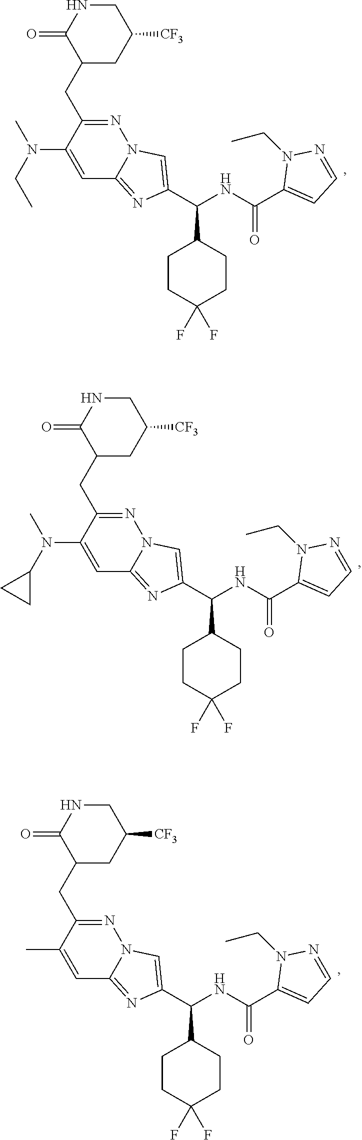 [SMILES: CCN(C)c1cc2nc([C@@H](NC(=O)c3ccnn3CC)C3CCC(F)(F)CC3)cn2nc1CC1C[C@@H](C(F)(F)F)CNC1=O ; CCn1nccc1C(=O)N[C@H](c1cn2nc(CC3C[C@@H](C(F)(F)F)CNC3=O)c(N(C)C3CC3)cc2n1)C1CCC(F)(F)CC1 ; CCn1nccc1C(=O)N[C@H](c1cn2nc(CC3C[C@H](C(F)(F)F)CNC3=O)c(C)cc2n1)C1CCC(F)(F)CC1]

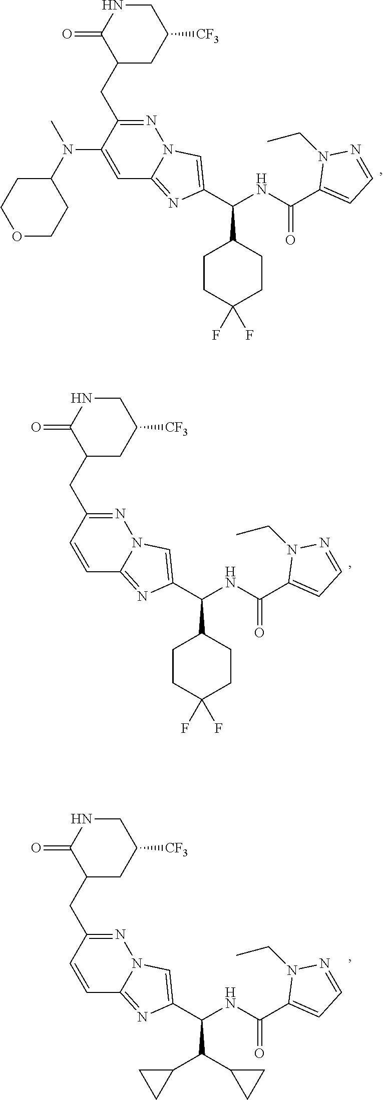 [SMILES: CCn1nccc1C(=O)N[C@H](c1cn2nc(CC3C[C@@H](C(F)(F)F)CNC3=O)c(N(C)C3CCOCC3)cc2n1)C1CCC(F)(F)CC1 ; CCn1nccc1C(=O)N[C@H](c1cn2nc(CC3C[C@@H](C(F)(F)F)CNC3=O)ccc2n1)C1CCC(F)(F)CC1 ; CCn1nccc1C(=O)N[C@H](c1cn2nc(CC3C[C@@H](C(F)(F)F)CNC3=O)ccc2n1)C(C1CC1)C1CC1]

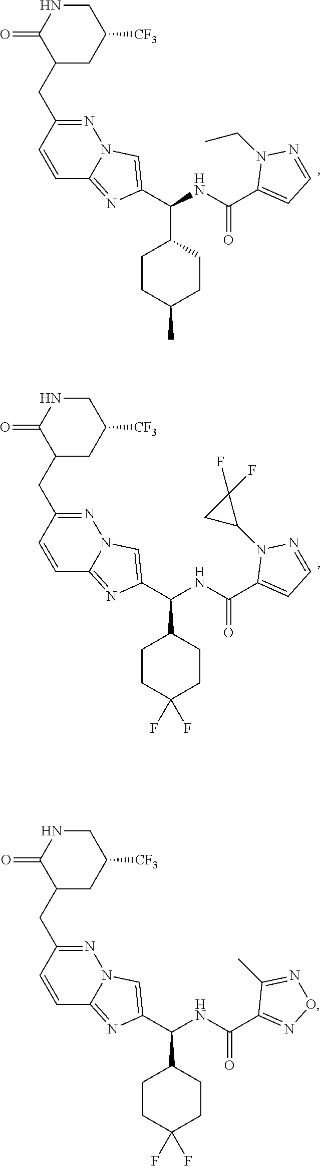 [SMILES: CCn1nccc1C(=O)N[C@H](c1cn2nc(CC3C[C@@H](C(F)(F)F)CNC3=O)ccc2n1)[C@H]1CC[C@H](C)CC1 ; O=C(N[C@H](c1cn2nc(CC3C[C@@H](C(F)(F)F)CNC3=O)ccc2n1)C1CCC(F)(F)CC1)c1ccnn1C1CC1(F)F ; Cc1nonc1C(=O)N[C@H](c1cn2nc(CC3C[C@@H](C(F)(F)F)CNC3=O)ccc2n1)C1CCC(F)(F)CC1]

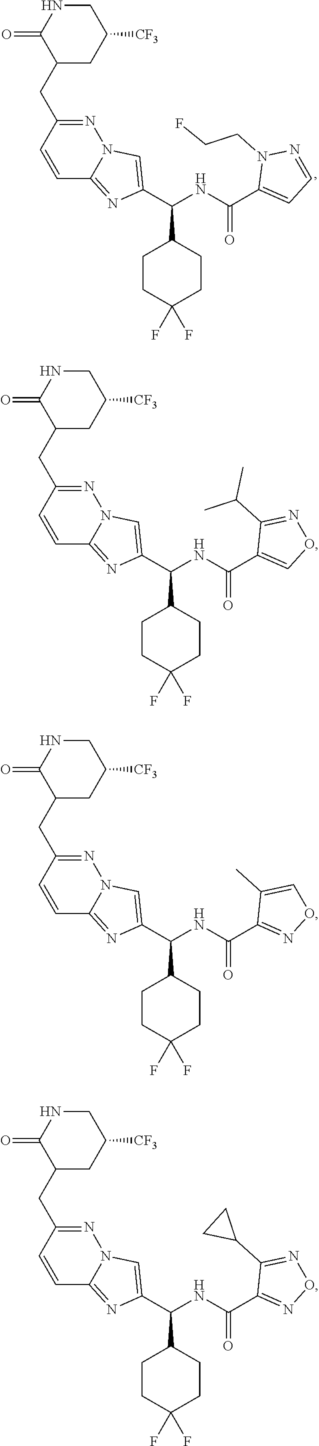 [SMILES: O=C(N[C@H](c1cn2nc(CC3C[C@@H](C(F)(F)F)CNC3=O)ccc2n1)C1CCC(F)(F)CC1)c1ccnn1CCF ; CC(C)c1nocc1C(=O)N[C@H](c1cn2nc(CC3C[C@@H](C(F)(F)F)CNC3=O)ccc2n1)C1CCC(F)(F)CC1 ; Cc1conc1C(=O)N[C@H](c1cn2nc(CC3C[C@@H](C(F)(F)F)CNC3=O)ccc2n1)C1CCC(F)(F)CC1 ; O=C(N[C@H](c1cn2nc(CC3C[C@@H](C(F)(F)F)CNC3=O)ccc2n1)C1CCC(F)(F)CC1)c1nonc1C1CC1]

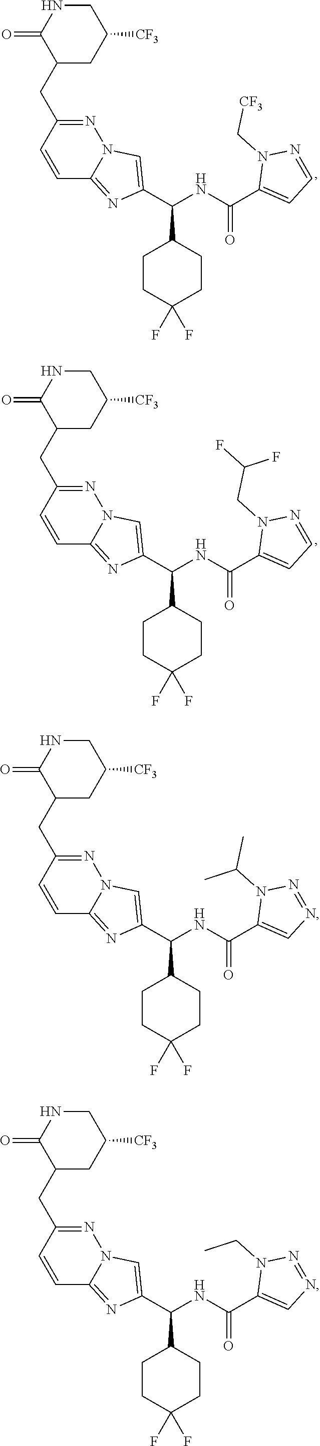 [SMILES: O=C(N[C@H](c1cn2nc(CC3C[C@@H](C(F)(F)F)CNC3=O)ccc2n1)C1CCC(F)(F)CC1)c1ccnn1CC(F)(F)F ; O=C(N[C@H](c1cn2nc(CC3C[C@@H](C(F)(F)F)CNC3=O)ccc2n1)C1CCC(F)(F)CC1)c1ccnn1CC(F)F ; CC(C)n1nncc1C(=O)N[C@H](c1cn2nc(CC3C[C@@H](C(F)(F)F)CNC3=O)ccc2n1)C1CCC(F)(F)CC1 ; CCn1nncc1C(=O)N[C@H](c1cn2nc(CC3C[C@@H](C(F)(F)F)CNC3=O)ccc2n1)C1CCC(F)(F)CC1]

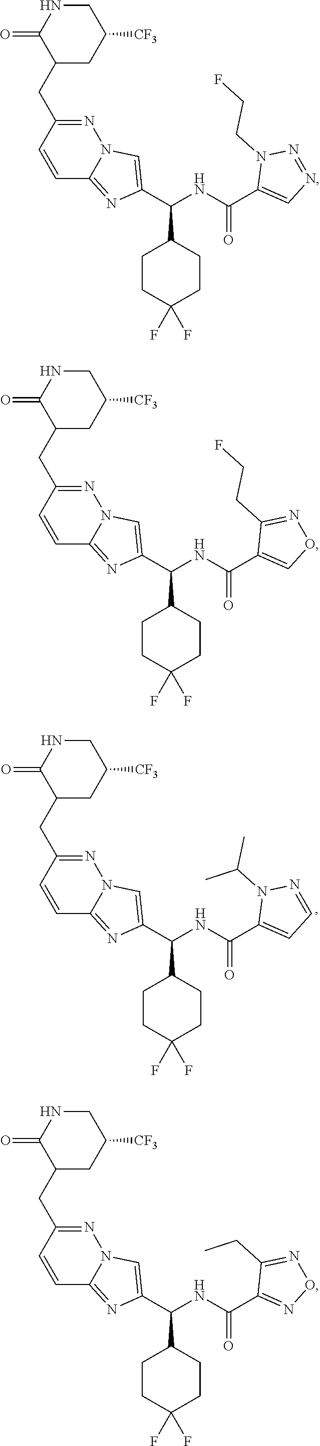 [SMILES: O=C(N[C@H](c1cn2nc(CC3C[C@@H](C(F)(F)F)CNC3=O)ccc2n1)C1CCC(F)(F)CC1)c1cnnn1CCF ; O=C(N[C@H](c1cn2nc(CC3C[C@@H](C(F)(F)F)CNC3=O)ccc2n1)C1CCC(F)(F)CC1)c1conc1CCF ; CC(C)n1nccc1C(=O)N[C@H](c1cn2nc(CC3C[C@@H](C(F)(F)F)CNC3=O)ccc2n1)C1CCC(F)(F)CC1 ; CCc1nonc1C(=O)N[C@H](c1cn2nc(CC3C[C@@H](C(F)(F)F)CNC3=O)ccc2n1)C1CCC(F)(F)CC1]

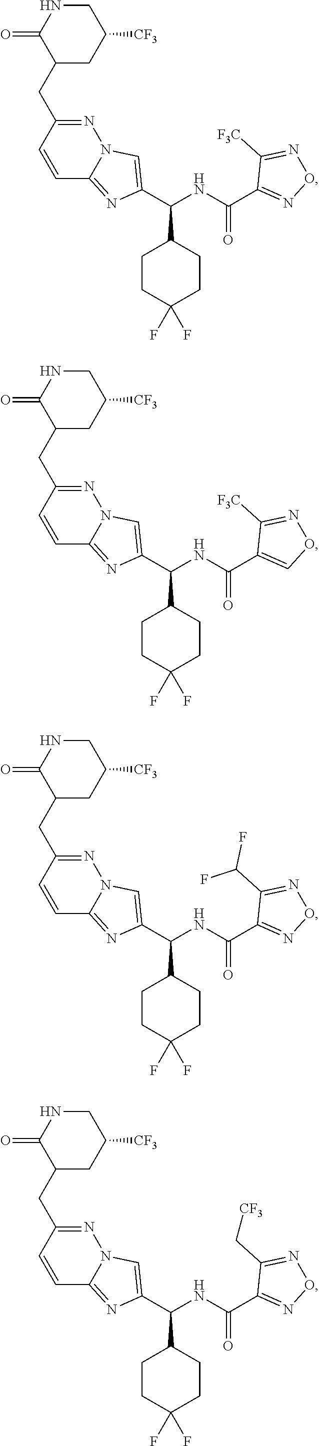 [SMILES: O=C(N[C@H](c1cn2nc(CC3C[C@@H](C(F)(F)F)CNC3=O)ccc2n1)C1CCC(F)(F)CC1)c1nonc1C(F)(F)F ; O=C(N[C@H](c1cn2nc(CC3C[C@@H](C(F)(F)F)CNC3=O)ccc2n1)C1CCC(F)(F)CC1)c1conc1C(F)(F)F ; O=C(N[C@H](c1cn2nc(CC3C[C@@H](C(F)(F)F)CNC3=O)ccc2n1)C1CCC(F)(F)CC1)c1nonc1C(F)F ; O=C(N[C@H](c1cn2nc(CC3C[C@@H](C(F)(F)F)CNC3=O)ccc2n1)C1CCC(F)(F)CC1)c1nonc1CC(F)(F)F]

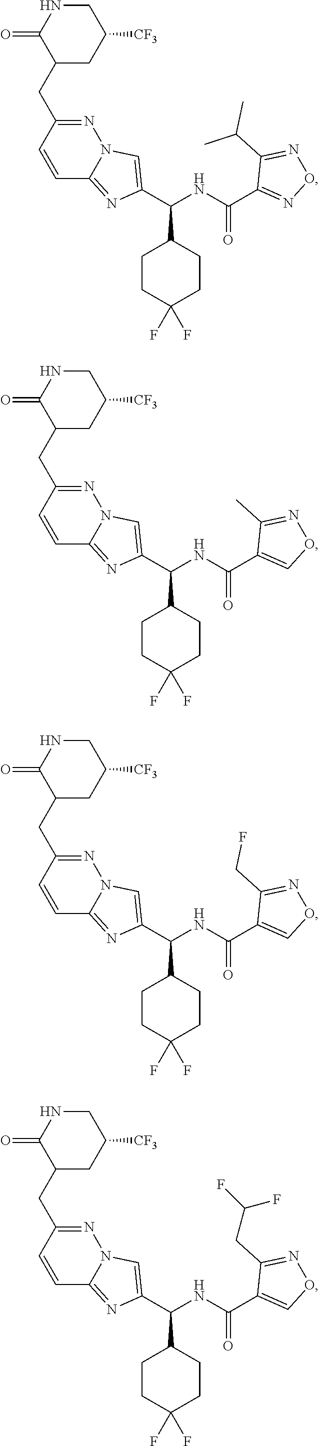 [SMILES: CC(C)c1nonc1C(=O)N[C@H](c1cn2nc(CC3C[C@@H](C(F)(F)F)CNC3=O)ccc2n1)C1CCC(F)(F)CC1 ; Cc1nocc1C(=O)N[C@H](c1cn2nc(CC3C[C@@H](C(F)(F)F)CNC3=O)ccc2n1)C1CCC(F)(F)CC1 ; O=C(N[C@H](c1cn2nc(CC3C[C@@H](C(F)(F)F)CNC3=O)ccc2n1)C1CCC(F)(F)CC1)c1conc1CF ; O=C(N[C@H](c1cn2nc(CC3C[C@@H](C(F)(F)F)CNC3=O)ccc2n1)C1CCC(F)(F)CC1)c1conc1CC(F)F]

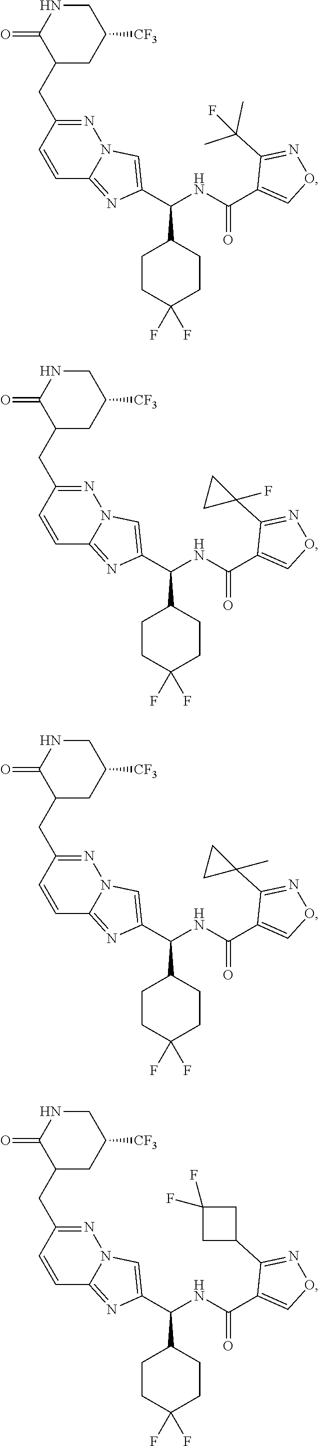 [SMILES: CC(C)(F)c1nocc1C(=O)N[C@H](c1cn2nc(CC3C[C@@H](C(F)(F)F)CNC3=O)ccc2n1)C1CCC(F)(F)CC1 ; O=C(N[C@H](c1cn2nc(CC3C[C@@H](C(F)(F)F)CNC3=O)ccc2n1)C1CCC(F)(F)CC1)c1conc1C1(F)CC1 ; CC1(c2nocc2C(=O)N[C@H](c2cn3nc(CC4C[C@@H](C(F)(F)F)CNC4=O)ccc3n2)C2CCC(F)(F)CC2)CC1 ; O=C(N[C@H](c1cn2nc(CC3C[C@@H](C(F)(F)F)CNC3=O)ccc2n1)C1CCC(F)(F)CC1)c1conc1C1CC(F)(F)C1]

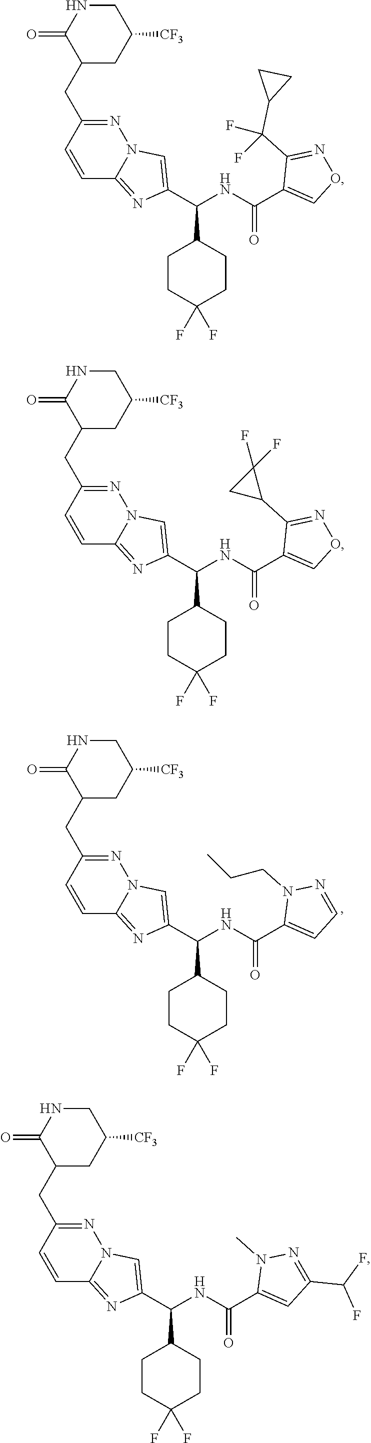 [SMILES: O=C(N[C@H](c1cn2nc(CC3C[C@@H](C(F)(F)F)CNC3=O)ccc2n1)C1CCC(F)(F)CC1)c1conc1C(F)(F)C1CC1 ; O=C(N[C@H](c1cn2nc(CC3C[C@@H](C(F)(F)F)CNC3=O)ccc2n1)C1CCC(F)(F)CC1)c1conc1C1CC1(F)F ; CCCn1nccc1C(=O)N[C@H](c1cn2nc(CC3C[C@@H](C(F)(F)F)CNC3=O)ccc2n1)C1CCC(F)(F)CC1 ; Cn1nc(C(F)F)cc1C(=O)N[C@H](c1cn2nc(CC3C[C@@H](C(F)(F)F)CNC3=O)ccc2n1)C1CCC(F)(F)CC1]

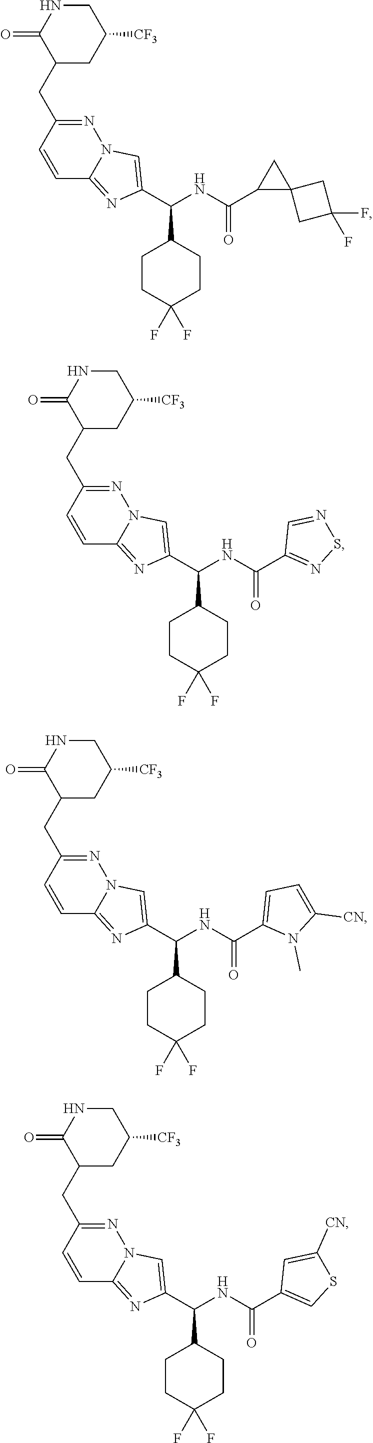 [SMILES: O=C1NC[C@H](C(F)(F)F)CC1Cc1ccc2nc([C@@H](NC(=O)C3CC34CC(F)(F)C4)C3CCC(F)(F)CC3)cn2n1 ; O=C(N[C@H](c1cn2nc(CC3C[C@@H](C(F)(F)F)CNC3=O)ccc2n1)C1CCC(F)(F)CC1)c1cnsn1 ; Cn1c(C#N)ccc1C(=O)N[C@H](c1cn2nc(CC3C[C@@H](C(F)(F)F)CNC3=O)ccc2n1)C1CCC(F)(F)CC1 ; N#Cc1cc(C(=O)N[C@H](c2cn3nc(CC4C[C@@H](C(F)(F)F)CNC4=O)ccc3n2)C2CCC(F)(F)CC2)cs1]

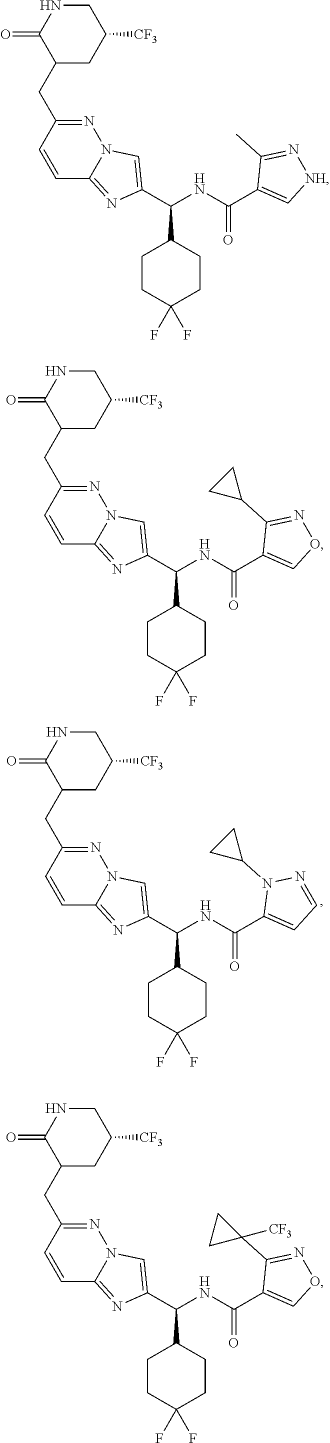 [SMILES: Cc1n[nH]cc1C(=O)N[C@H](c1cn2nc(CC3C[C@@H](C(F)(F)F)CNC3=O)ccc2n1)C1CCC(F)(F)CC1 ; O=C(N[C@H](c1cn2nc(CC3C[C@@H](C(F)(F)F)CNC3=O)ccc2n1)C1CCC(F)(F)CC1)c1conc1C1CC1 ; O=C(N[C@H](c1cn2nc(CC3C[C@@H](C(F)(F)F)CNC3=O)ccc2n1)C1CCC(F)(F)CC1)c1ccnn1C1CC1 ; O=C(N[C@H](c1cn2nc(CC3C[C@@H](C(F)(F)F)CNC3=O)ccc2n1)C1CCC(F)(F)CC1)c1conc1C1(C(F)(F)F)CC1]

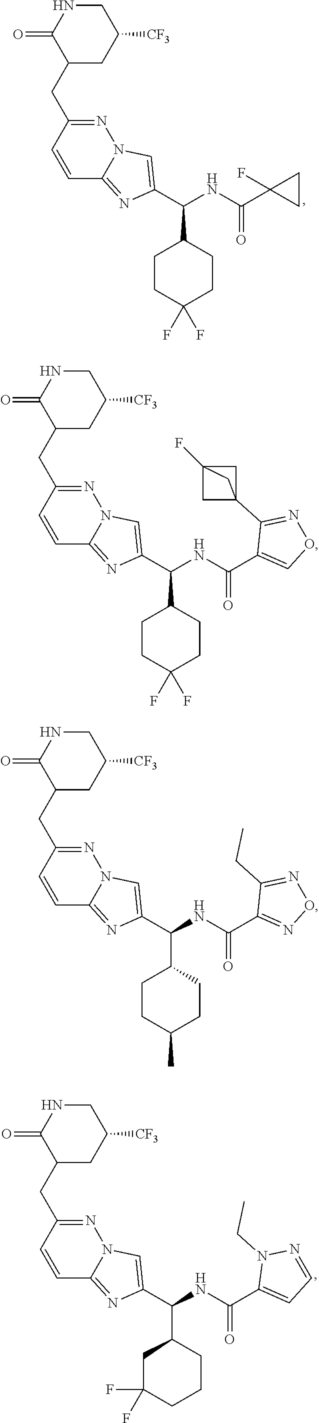 [SMILES: O=C1NC[C@H](C(F)(F)F)CC1Cc1ccc2nc([C@@H](NC(=O)C3(F)CC3)C3CCC(F)(F)CC3)cn2n1 ; O=C(N[C@H](c1cn2nc(CC3C[C@@H](C(F)(F)F)CNC3=O)ccc2n1)C1CCC(F)(F)CC1)c1conc1C12CC(F)(C1)C2 ; CCc1nonc1C(=O)N[C@H](c1cn2nc(CC3C[C@@H](C(F)(F)F)CNC3=O)ccc2n1)[C@H]1CC[C@H](C)CC1 ; CCn1nccc1C(=O)N[C@H](c1cn2nc(CC3C[C@@H](C(F)(F)F)CNC3=O)ccc2n1)[C@H]1CCCC(F)(F)C1]

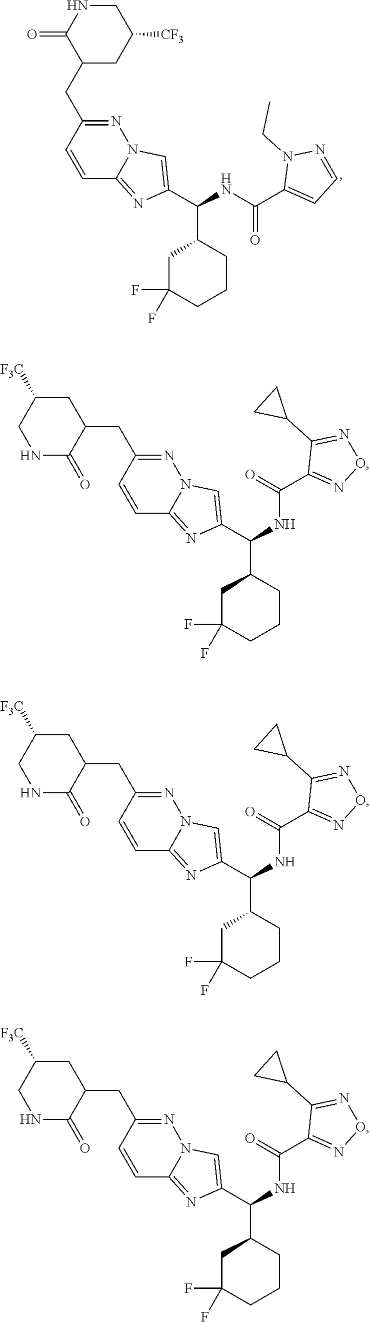 [SMILES: CCn1nccc1C(=O)N[C@H](c1cn2nc(CC3C[C@@H](C(F)(F)F)CNC3=O)ccc2n1)[C@@H]1CCCC(F)(F)C1 ; O=C(N[C@H](c1cn2nc(CC3C[C@@H](C(F)(F)F)CNC3=O)ccc2n1)[C@H]1CCCC(F)(F)C1)c1nonc1C1CC1 ; O=C(N[C@H](c1cn2nc(CC3C[C@@H](C(F)(F)F)CNC3=O)ccc2n1)[C@@H]1CCCC(F)(F)C1)c1nonc1C1CC1 ; O=C(N[C@H](c1cn2nc(CC3C[C@@H](C(F)(F)F)CNC3=O)ccc2n1)[C@H]1CCCC(F)(F)C1)c1nonc1C1CC1]

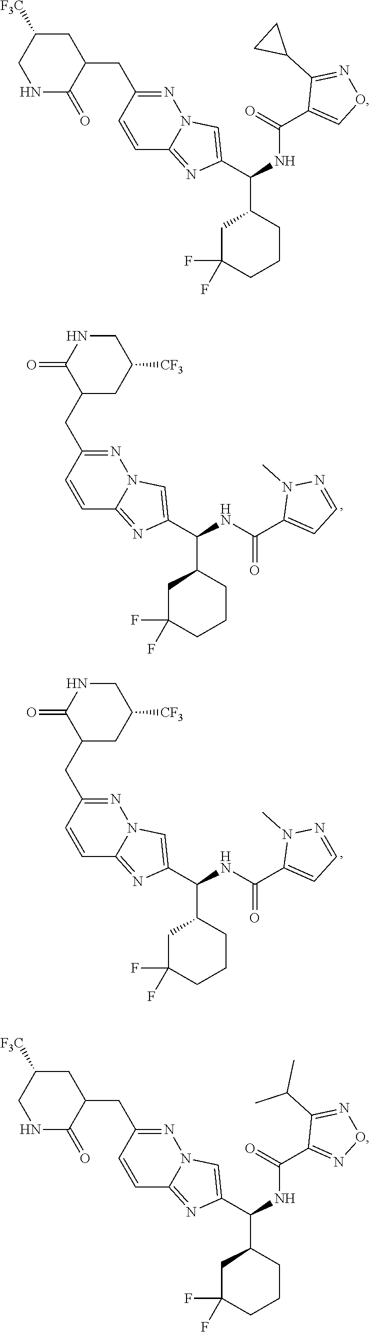 [SMILES: O=C(N[C@H](c1cn2nc(CC3C[C@@H](C(F)(F)F)CNC3=O)ccc2n1)[C@@H]1CCCC(F)(F)C1)c1conc1C1CC1 ; Cn1nccc1C(=O)N[C@H](c1cn2nc(CC3C[C@@H](C(F)(F)F)CNC3=O)ccc2n1)[C@H]1CCCC(F)(F)C1 ; Cn1nccc1C(=O)N[C@H](c1cn2nc(CC3C[C@@H](C(F)(F)F)CNC3=O)ccc2n1)[C@@H]1CCCC(F)(F)C1 ; CC(C)c1nonc1C(=O)N[C@H](c1cn2nc(CC3C[C@@H](C(F)(F)F)CNC3=O)ccc2n1)[C@H]1CCCC(F)(F)C1]

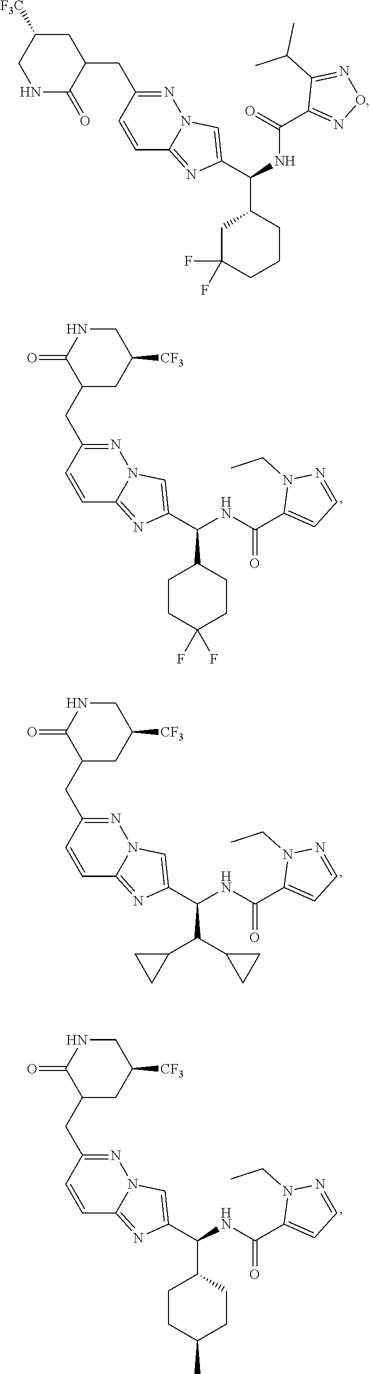 [SMILES: CC(C)c1nonc1C(=O)N[C@H](c1cn2nc(CC3C[C@@H](C(F)(F)F)CNC3=O)ccc2n1)[C@@H]1CCCC(F)(F)C1 ; CCn1nccc1C(=O)N[C@H](c1cn2nc(CC3C[C@H](C(F)(F)F)CNC3=O)ccc2n1)C1CCC(F)(F)CC1 ; CCn1nccc1C(=O)N[C@H](c1cn2nc(CC3C[C@H](C(F)(F)F)CNC3=O)ccc2n1)C(C1CC1)C1CC1 ; CCn1nccc1C(=O)N[C@H](c1cn2nc(CC3C[C@H](C(F)(F)F)CNC3=O)ccc2n1)[C@H]1CC[C@H](C)CC1]

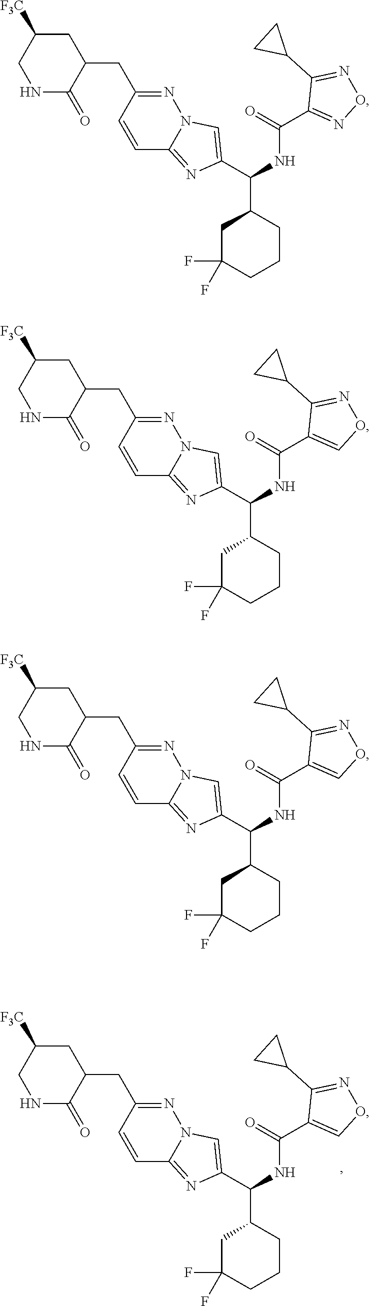 [SMILES: O=C(N[C@H](c1cn2nc(CC3C[C@H](C(F)(F)F)CNC3=O)ccc2n1)[C@H]1CCCC(F)(F)C1)c1nonc1C1CC1 ; O=C(N[C@H](c1cn2nc(CC3C[C@H](C(F)(F)F)CNC3=O)ccc2n1)[C@@H]1CCCC(F)(F)C1)c1conc1C1CC1 ; O=C(N[C@H](c1cn2nc(CC3C[C@H](C(F)(F)F)CNC3=O)ccc2n1)[C@H]1CCCC(F)(F)C1)c1conc1C1CC1 ; O=C(N[C@H](c1cn2nc(CC3C[C@H](C(F)(F)F)CNC3=O)ccc2n1)[C@@H]1CCCC(F)(F)C1)c1conc1C1CC1]

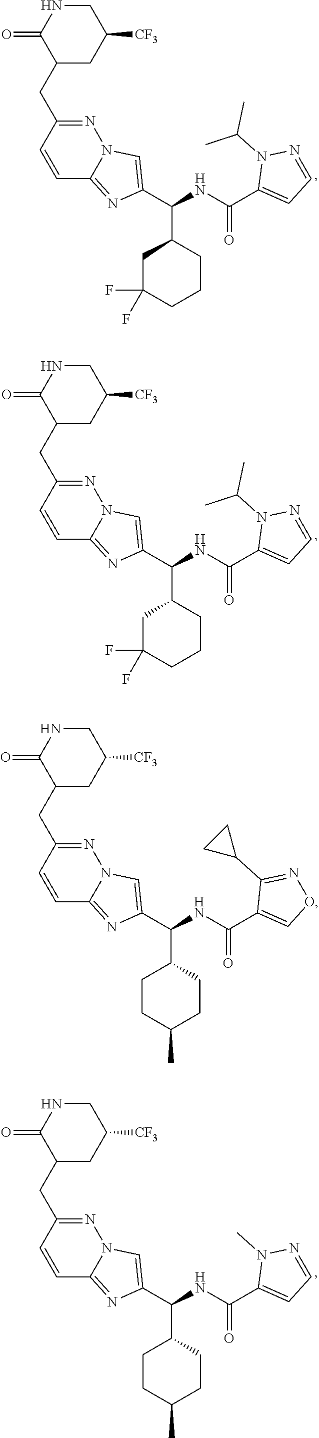 [SMILES: CC(C)n1nccc1C(=O)N[C@H](c1cn2nc(CC3C[C@H](C(F)(F)F)CNC3=O)ccc2n1)[C@H]1CCCC(F)(F)C1 ; CC(C)n1nccc1C(=O)N[C@H](c1cn2nc(CC3C[C@H](C(F)(F)F)CNC3=O)ccc2n1)[C@@H]1CCCC(F)(F)C1 ; C[C@H]1CC[C@H]([C@H](NC(=O)c2conc2C2CC2)c2cn3nc(CC4C[C@@H](C(F)(F)F)CNC4=O)ccc3n2)CC1 ; Cn1nccc1C(=O)N[C@H](c1cn2nc(CC3C[C@@H](C(F)(F)F)CNC3=O)ccc2n1)[C@H]1CC[C@H](C)CC1]

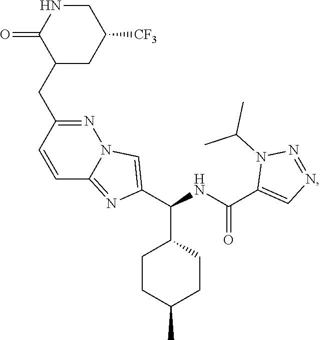 [SMILES: CC(C)n1nncc1C(=O)N[C@H](c1cn2nc(CC3C[C@@H](C(F)(F)F)CNC3=O)ccc2n1)[C@H]1CC[C@H](C)CC1]

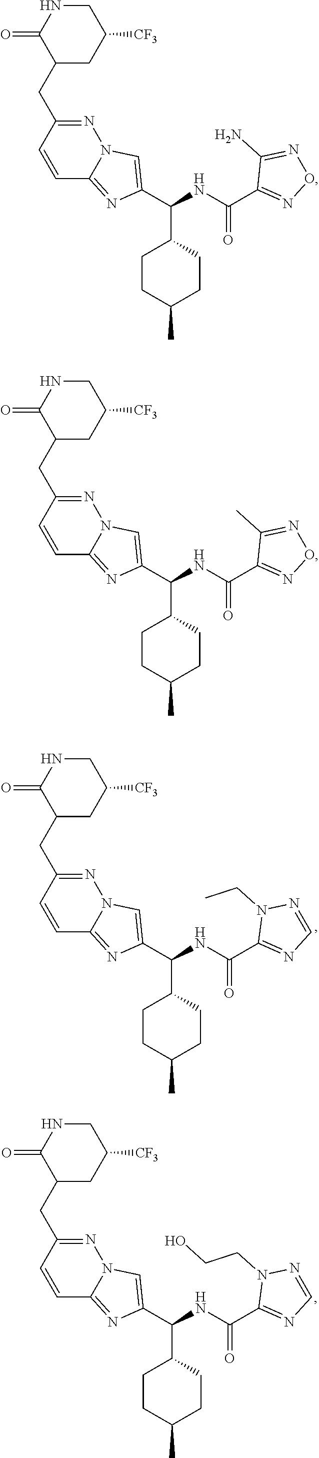 [SMILES: C[C@H]1CC[C@H]([C@H](NC(=O)c2nonc2N)c2cn3nc(CC4C[C@@H](C(F)(F)F)CNC4=O)ccc3n2)CC1 ; Cc1nonc1C(=O)N[C@H](c1cn2nc(CC3C[C@@H](C(F)(F)F)CNC3=O)ccc2n1)[C@H]1CC[C@H](C)CC1 ; CCn1ncnc1C(=O)N[C@H](c1cn2nc(CC3C[C@@H](C(F)(F)F)CNC3=O)ccc2n1)[C@H]1CC[C@H](C)CC1 ; C[C@H]1CC[C@H]([C@H](NC(=O)c2ncnn2CCO)c2cn3nc(CC4C[C@@H](C(F)(F)F)CNC4=O)ccc3n2)CC1]

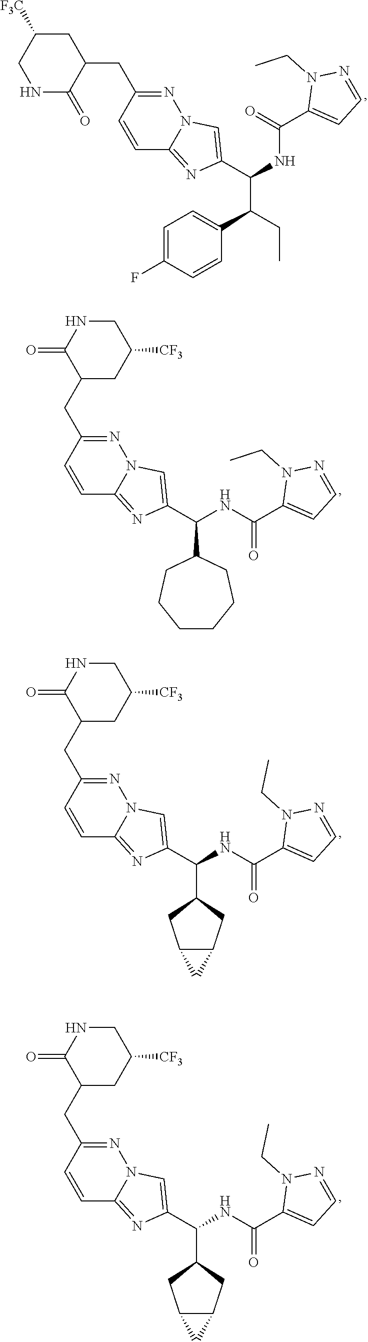 [SMILES: CC[C@@H](c1ccc(F)cc1)[C@H](NC(=O)c1ccnn1CC)c1cn2nc(CC3C[C@@H](C(F)(F)F)CNC3=O)ccc2n1 ; CCn1nccc1C(=O)N[C@H](c1cn2nc(CC3C[C@@H](C(F)(F)F)CNC3=O)ccc2n1)C1CCCCCC1 ; CCn1nccc1C(=O)N[C@H](c1cn2nc(CC3C[C@@H](C(F)(F)F)CNC3=O)ccc2n1)[C@@H]1C[C@H]2C[C@H]2C1 ; CCn1nccc1C(=O)N[C@@H](c1cn2nc(CC3C[C@@H](C(F)(F)F)CNC3=O)ccc2n1)[C@@H]1C[C@H]2C[C@H]2C1]

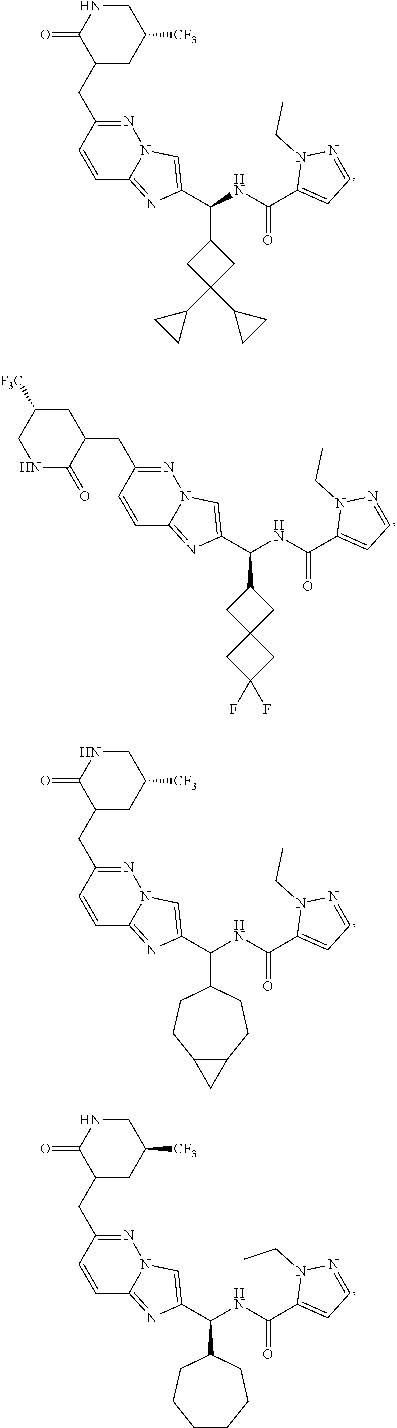 [SMILES: CCn1nccc1C(=O)N[C@H](c1cn2nc(CC3C[C@@H](C(F)(F)F)CNC3=O)ccc2n1)C1CC(C2CC2)(C2CC2)C1 ; CCn1nccc1C(=O)N[C@H](c1cn2nc(CC3C[C@@H](C(F)(F)F)CNC3=O)ccc2n1)C1CC2(C1)CC(F)(F)C2 ; CCn1nccc1C(=O)NC(c1cn2nc(CC3C[C@@H](C(F)(F)F)CNC3=O)ccc2n1)C1CCC2CC2CC1 ; CCn1nccc1C(=O)N[C@H](c1cn2nc(CC3C[C@H](C(F)(F)F)CNC3=O)ccc2n1)C1CCCCCC1]

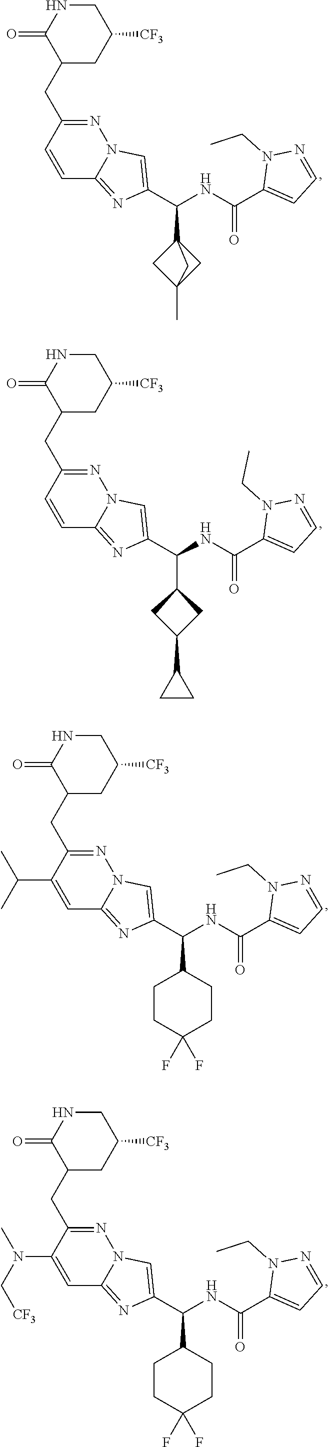 [SMILES: CCn1nccc1C(=O)N[C@H](c1cn2nc(CC3C[C@@H](C(F)(F)F)CNC3=O)ccc2n1)C12CC(C)(C1)C2 ; CCn1nccc1C(=O)N[C@H](c1cn2nc(CC3C[C@@H](C(F)(F)F)CNC3=O)ccc2n1)[C@H]1C[C@@H](C2CC2)C1 ; CCn1nccc1C(=O)N[C@H](c1cn2nc(CC3C[C@@H](C(F)(F)F)CNC3=O)c(C(C)C)cc2n1)C1CCC(F)(F)CC1 ; CCn1nccc1C(=O)N[C@H](c1cn2nc(CC3C[C@@H](C(F)(F)F)CNC3=O)c(N(C)CC(F)(F)F)cc2n1)C1CCC(F)(F)CC1]

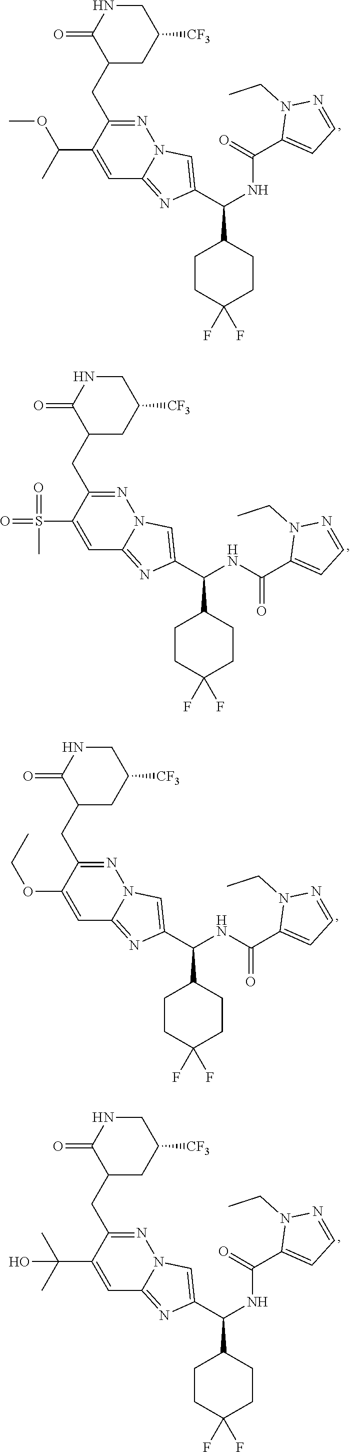 [SMILES: CCn1nccc1C(=O)N[C@H](c1cn2nc(CC3C[C@@H](C(F)(F)F)CNC3=O)c(C(C)OC)cc2n1)C1CCC(F)(F)CC1 ; CCn1nccc1C(=O)N[C@H](c1cn2nc(CC3C[C@@H](C(F)(F)F)CNC3=O)c(S(C)(=O)=O)cc2n1)C1CCC(F)(F)CC1 ; CCOc1cc2nc([C@@H](NC(=O)c3ccnn3CC)C3CCC(F)(F)CC3)cn2nc1CC1C[C@@H](C(F)(F)F)CNC1=O ; CCn1nccc1C(=O)N[C@H](c1cn2nc(CC3C[C@@H](C(F)(F)F)CNC3=O)c(C(C)(C)O)cc2n1)C1CCC(F)(F)CC1]

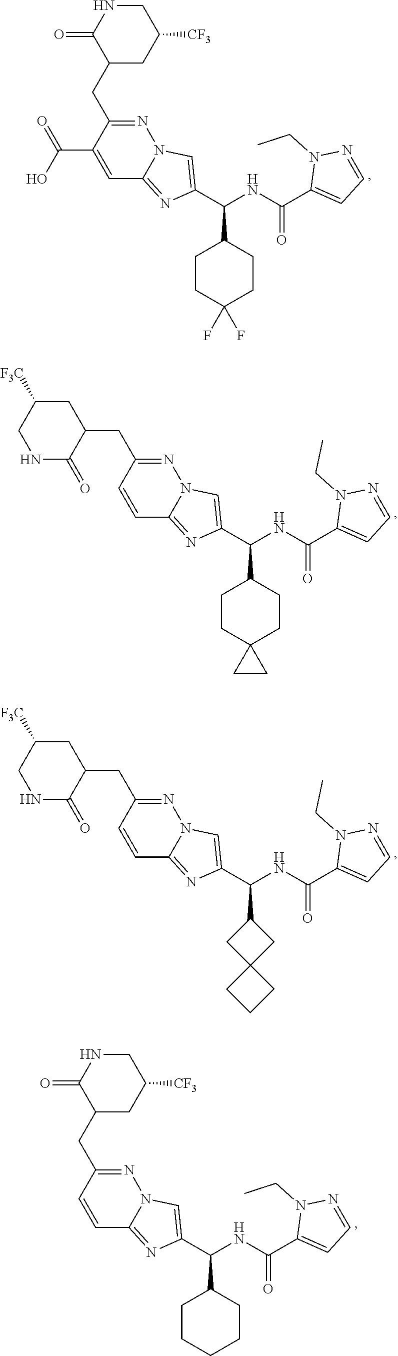 [SMILES: CCn1nccc1C(=O)N[C@H](c1cn2nc(CC3C[C@@H](C(F)(F)F)CNC3=O)c(C(=O)O)cc2n1)C1CCC(F)(F)CC1 ; CCn1nccc1C(=O)N[C@H](c1cn2nc(CC3C[C@@H](C(F)(F)F)CNC3=O)ccc2n1)C1CCC2(CC1)CC2 ; CCn1nccc1C(=O)N[C@H](c1cn2nc(CC3C[C@@H](C(F)(F)F)CNC3=O)ccc2n1)C1CC2(CCC2)C1 ; CCn1nccc1C(=O)N[C@H](c1cn2nc(CC3C[C@@H](C(F)(F)F)CNC3=O)ccc2n1)C1CCCCC1]

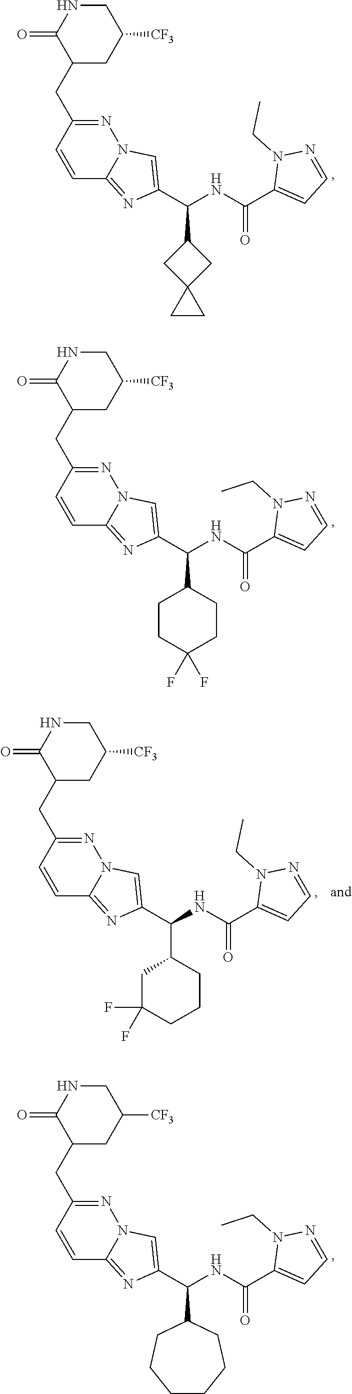 [SMILES: CCn1nccc1C(=O)N[C@H](c1cn2nc(CC3C[C@@H](C(F)(F)F)CNC3=O)ccc2n1)C1CC2(CC2)C1 ; CCn1nccc1C(=O)N[C@H](c1cn2nc(CC3C[C@@H](C(F)(F)F)CNC3=O)ccc2n1)C1CCC(F)(F)CC1 ; CCn1nccc1C(=O)N[C@H](c1cn2nc(CC3C[C@@H](C(F)(F)F)CNC3=O)ccc2n1)[C@@H]1CCCC(F)(F)C1 ; CCn1nccc1C(=O)N[C@H](c1cn2nc(CC3CC(C(F)(F)F)CNC3=O)ccc2n1)C1CCCCCC1]

or pharmaceutically acceptable salts thereof.

### Lactam Substituted 7-Imidazopyridazine IL-17A Modulators

In some aspects, the present disclosure provides a compound represented by structure of

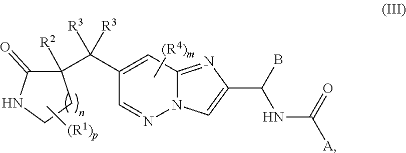

or a pharmaceutically acceptable salt thereof wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
   C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂,
   —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
   C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN;
B is selected from —C(H)(R⁵)₂ and C₃₋₁₀ carbocycle, wherein each C₃₋₁₀ carbocycle is optionally substituted with one or more substituents independently selected from:
   halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
   C₁₋₁₀ alkyl and C₃₋₁₀ carbocycle, any of which is optionally substituted with one or more substituents independently selected from halogen, —OR¹², —SR¹², —N(R¹²)₂,
   —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹²,
   —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁵ is independently selected at each occurrence from (i), (ii), and (iii):
   (i) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³,
      —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (iii) C₃₋₁₀ carbocycle optionally substituted with one or more substituents independently selected from:
      halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂,
      —N(R¹³)C(O)R¹³, —C(O)OR¹³, —OC(O)R¹³, —S(O)R¹³, —S(O)₂R¹³, —NO₂, ═O, ═S, ═N(R¹³), and —CN;
each R¹ is independently selected at each occurrence from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴,
   —S(O)₂R¹⁴, —NO₂, ═O, ═S, ═N(R¹⁴), —CN; and C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN;
R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN; or
each R⁴ is independently selected at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷,
   —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN;
R¹¹, R^{11A}, R¹², R¹³, R¹⁴, R¹⁵, R¹⁶, and R¹⁷ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2;
m is selected from 0, 1 and 2; and
p is selected from 0, 1, 2, 3, 4, 5, and 6.

In some embodiments, for the compound or salt of Formula (III), A is selected from 5- to 6-membered heteroaryl and C₃₋₆ carbocycle, each of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
C₁₋₆ alkyl optionally substituted with one or more substituents independently selected from halogen, —OR¹¹, —SR¹¹, —N(R¹¹)₂, —C(O)R¹¹, —C(O)N(R¹¹)₂,
—N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

In some embodiments, for the compound or salt of Formula (III), A is selected from 5- to 6-membered heteroaryl and C₃₋₆ carbocycle, each of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
C₃₋₁₀ carbocycle optionally substituted with one or more substituents independently selected from halogen, —OR^{11A}, SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

In some embodiments, for the compound or salt of Formula (III), A is selected from 5- to 6-membered heteroaryl and C₃₋₆ carbocycle, each of which is optionally substituted with one or more substituents independently selected from:
C₁₋₆ alkyl optionally substituted with one or more substituents independently selected from halogen, —OR¹¹, —SR¹¹, —N(R¹¹)₂, —C(O)R¹¹, —C(O)N(R¹¹)₂,
—N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
C₃₋₁₀ carbocycle optionally substituted with one or more substituents independently selected from halogen, —OR^{11A}, SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

In some embodiments, for the compound or salt of Formula (III), A is selected from 5- to 6-membered heteroaryl and C₃₋₆ carbocycle, any of which is optionally substituted with one or more substituents independently selected from C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN. In some embodiments, A is selected from 5- to 6-membered heteroaryl and C₃₋₆ carbocycle, any of which is optionally substituted with one or more substituents independently selected from C₃₋₁₀ carbocycle optionally substituted with one or more substituents independently selected from halogen, —OR^{11A}, —SR¹A, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), and —CN. In some embodiments, A is selected from 5- to 6-membered heteroayl and C₃₋₆ carbocycle, any of which is optionally substituted with one or more substituents independently selected from C₃₋₁₀carbocycle optionally substituted with one or more C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}) and —CN.

In some embodiments, for the compound or salt of Formula (III), A is selected from 5- to 6-membered heteroaryl optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂,
—N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

In some embodiments, for the compound or salt of Formula (III), A is selected from 5- to 6-membered heteroaryl optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
C₃₋₁₀ carbocycle optionally substituted with one or more substituents independently selected from halogen, —OR^{11A}, SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

In some embodiments, for the compound or salt of Formula (III), A is selected from 5- to 6-membered heteroaryl optionally substituted with one or more substituents independently selected from:
C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂,
—N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
C₃₋₁₀ carbocycle optionally substituted with one or more substituents independently selected from halogen, —OR^{11A}, SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

In some embodiments, for the compound or salt of Formula (III), A is 5- to 6-membered heteroaryl optionally substituted with halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), and —CN.

In some embodiments, for the compound or salt of Formula (III), A is 5- to 6-membered heteroaryl optionally substituted with C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN. In some embodiments, A is 5- to 6-membered heteroaryl optionally substituted with C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), and —CN. In some embodiments, A is 5- to 6-membered heteroaryl optionally substituted with C₃₋₁₀ carbocycle optionally substituted with one or more C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

In some embodiments, for the compound or salt of Formula (III), A is 5-membered heteroaryl optionally substituted with C₃₋₁₀ carbocycle optionally substituted with one or more substituents independently selected from halogen, —OR^{11A}, —N(R^{11A})₂, —C(O)R^{11A}, —NO₂, —CN; and C₁₋₁₀ alkyl optionally substituted with one or more substituents selected from: halogen, —OR^{11A}, —N(R^{11A})₂, —C(O)R^{11A}, —NO₂, and —CN. In some embodiments, A is 5-membered heteroayl optionally substituted with C₃₋₁₀ carbocycle optionally substituted with one or more substituents independently selected from halogen, —OR^{11A}, —N(R^{11A})₂, —C(O)R^{11A}, —NO₂, —CN. In some embodiments, A is 5-membered heteroaryl optionally substituted with C₃₋₁₀carbocycle optionally substituted with C₁₋₁₀ alkyl optionally substituted with one or more substituents selected from: halogen, —OR^{11A}, —N(R^{11A})₂, —C(O)R^{11A}, —NO₂, and —CN.

In some embodiments, for the compound or salt of Formula (III), A is 5-membered heteroaryl optionally substituted with C₃₋₆ carbocycle optionally substituted with one or more substituents independently selected from halogen, —OR^{11A}, —N(R^{11A})₂, —C(O)R^{11A}, —NO₂, —CN; and C₁₋₆ alkyl optionally substituted with one or more substituents selected from: halogen, —OR^{11A}, —N(R^{11A})₂, —C(O)R^{11A}, —NO₂, and —CN. In some embodiments, A is selected from pyrazolyl, imidazolyl, oxazolyl, isoxazolyl, oxadiazolyl, each of which optionally substituted with C₃₋₆ cycloalkyl optionally substituted with one or more substituents independently selected from halogen, —OR^{11A}, —N(R^{11A})₂, —C(O)R^{11A}, —NO₂, —CN, C₁₋₆ alkyl, and C₁₋₆ haloalkyl.

In some embodiments, for the compound or salt of Formula (III), A is oxazolyl, isoxazolyl, oxadiazolyl, each of which is optionally substituted with C₃₋₆ cycloalkyl optionally substituted with one or more substituents independently selected from halogen, —OR^{11A}, —N(R^{11A})₂, —C(O)R^{11A}, —NO₂, —CN, C₁₋₆ alkyl, and C₁₋₆ haloalkyl. In some embodiments, for the compound or salt of Formula (III), A is oxazolyl, isoxazolyl, oxadiazolyl, each of which is optionally substituted with C₃₋₆ cycloalkyl optionally substituted with one or more substituents independently selected from halogen, —OR^{11A}, —N(R^{11A})₂, —C(O)R^{11A}, —NO₂, —CN, C₁₋₆ alkyl, and C₁₋₆ haloalkyl. In some embodiments, for the compound or salt of Formula (III), A is oxazolyl, isoxazolyl, oxadiazolyl, each of which is optionally substituted with cyclopropyl, cyclobutyl, cyclopentyl, or cyclohexyl, each of which is optionally substituted with one or more substituents independently selected from halogen, —OR^{11A}, —N(R^{11A})₂, —C(O)R^{11A}, —NO₂, —CN, C₁₋₆ alkyl, and C₁₋₆ haloalkyl. In some embodiments, for the compound or salt of Formula (III), A is oxazolyl, isoxazolyl, oxadiazolyl, each of which is substituted one or more substituents independently selected from: cyclopropyl, cyclobutyl, cyclopentyl, and cyclohexyl, any of which is optionally substituted with one or more substituents independently selected from halogen, —OR^{11A}, —N(R^{11A})₂, —C(O)R^{11A}, —NO₂, —CN, C₁₋₆ alkyl, and C₁₋₆ haloalkyl.

In some embodiments, for the compound or salt of Formula (III), A is selected from:

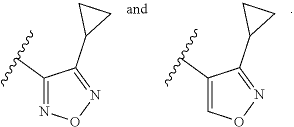 [SMILES: *c1nonc1C1CC1 ; *c1conc1C1CC1]

In some embodiments, A is

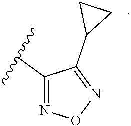 [SMILES: *c1nonc1C1CC1]

In some embodiments, A is

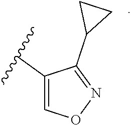 [SMILES: *c1conc1C1CC1]

In some aspects, the present disclosure provides a compound represented by structure of Formula (IV):

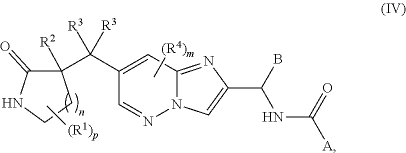

or a pharmaceutically acceptable salt thereof wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
   C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂,
   —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN;
B is selected from —C(H)(R⁵)₂ and C₃₋₁₀ carbocycle, wherein each C₃₋₁₀ carbocycle is optionally substituted with one or more substituents independently selected from:
   halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
   C₁₋₁₀ alkyl and C₃₋₁₀ carbocycle, any of which is optionally substituted with one or more substituents independently selected from halogen, —OR¹², —SR¹², —N(R¹²)₂,
   —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹²,
   —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁵ is independently selected at each occurrence from (i), (ii), and (iii):
   (i) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³,
      —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (iii) C₃₋₁₀ carbocycle optionally substituted with one or more substituents independently selected from:
      halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂,
      —N(R¹³)C(O)R¹³, —C(O)OR¹³, —OC(O)R¹³, —S(O)R¹³, —S(O)₂R¹³, —NO₂, ═O, ═S, ═N(R¹³), and —CN;
each R¹ is independently selected at each occurrence from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴,
   —S(O)₂R¹⁴, —NO₂, ═O, ═S, ═N(R¹⁴), —CN; and C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN;
R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN; or
each R⁴ is independently selected at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷,
   —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN;
R¹¹, R¹², R¹³, R¹⁴, R¹⁵, R¹⁶, and R¹⁷ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2;
m is selected from 0, 1 and 2; and
p is selected from 0, 1, 2, 3, 4, 5, and 6.

In some embodiments, for the compound or salt of Formula (III) or (IV), n is 2.

In some embodiments, for the compound or salt of Formula (III) or (IV), n is 1.

In certain embodiments, the structure of Formula (IV) is represented by Formula (IV-a):

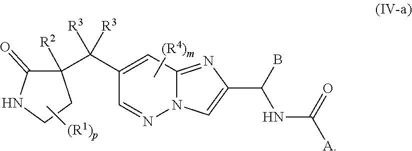

In certain embodiments, the structure of Formula (IV) is represented by Formula (IV-a):

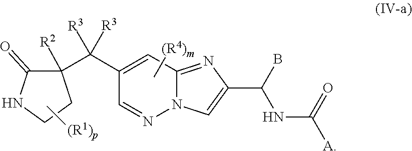

In certain embodiments, the structure of Formula (IV) is represented by Formula (IV-a):

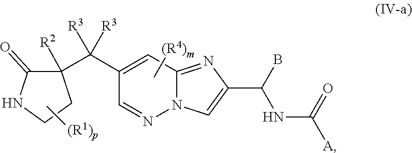

wherein R¹, R², R³, R⁴, A, B, p, and m are each defined as in Formula (III).

In certain embodiments, the structure of Formula (IV) is represented by Formula (IV-a):

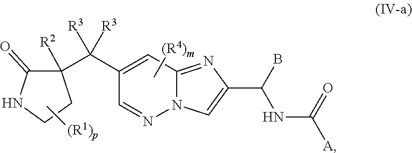

wherein R¹, R², R³, R⁴, A, B, p, and m are each defined as in Formula (IV).

In certain embodiments, the structure of Formula (IV) is represented by Formula (IV-b):

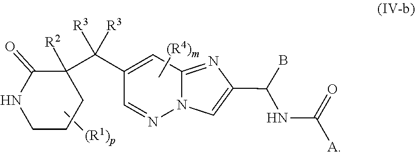

In certain embodiments, the structure of Formula (IV) is represented by Formula (IV-b):

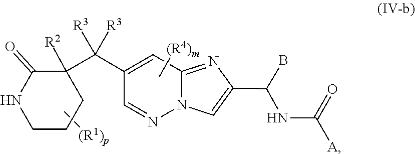

wherein R¹, R², R³, R⁴, A, B, p, and m are each defined as in Formula (III).

In certain embodiments, the structure of Formula (IV) is represented by Formula (IV-b):

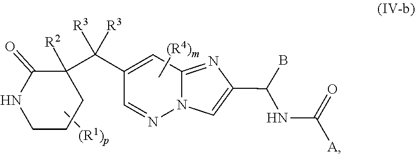

wherein R¹, R², R³, R⁴, A, B, p, and m are each defined as in Formula (IV).

In some embodiments, for the compound or salt of Formula (III), (IV), (IV-a) and (IV-b), p is selected from 0, 1, 2, 3, 4, 5, and 6. In some embodiments, p is selected from 1, 2, 3, 4, 5, and 6. In some embodiments, p is selected from 0, 1, 2, 3, 4, and 5. In some embodiments, p is selected from 0, 1, 2, 3, and 4. In some embodiments, p is selected from 0, 1, 2, and 3. In some embodiments, p is selected from 0, 1, and 2. In some embodiments, p is selected from 0 and 1. In some embodiments, p is selected from 1, 2, 3, 4 and 5. In some embodiments, p is selected from 2, 3, 4 and 5. In some embodiments, p is selected from 3, 4 and 5. In some embodiments, p is selected from 4 and 5. In some embodiments, p is 0. In some embodiments, p is 1. In some embodiments, p is 2. In some embodiments, p is 3. In some embodiments, p is 4. In some embodiments, p is 5. In some embodiments, p is 6.

In some embodiments, for the compound or salt of Formula (III), (IV), (IV-a) and (IV-b), R¹ is selected from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN; and C₁₋₆ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN. In some embodiments, R¹ is selected from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), and —CN. In some embodiments, R¹ is selected from C₁₋₆ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN. In some embodiments, R¹ is selected from halogen, —OR¹⁴, —N(R¹⁴)₂, —NO₂, —CN, C₁₋₆ alkyl, and C₁₋₆ haloalkyl. In some embodiments, R¹ is selected from halogen and —CF₃. In some embodiments, R¹ is —CF₃.

In some embodiments, for the compound or salt of Formula (III), (IV), (IV-a) and (IV-b), R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN, and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN. In some embodiments, R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, and —CN. In some embodiments, R² is selected from C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN. In some embodiments, R² is selected from hydrogen, halogen, —OR¹⁵, —N(R¹⁵)₂, —C(O)R¹⁵, —NO₂, —CN, C₁₋₃ alkyl, and C₁₋₃ haloalkyl. In some embodiments, R² is selected from hydrogen, halogen, and C₁₋₃ alkyl. In some embodiments, R² is selected from hydrogen, halogen, methyl, and ethyl. In some embodiments, R² is selected from hydrogen and methyl. In some embodiments, R² is hydrogen.

In some embodiments, for the compound or salt of Formula (III), (IV), (IV-a) and (IV-b), each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN, and C₁₋₆ alkyl optionally substituted with one or more substituents independently selected from halogen, —OR¹⁶, —SR¹⁶, —N(R¹⁶)₂, —C(O)R¹⁶, and —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN. In some embodiments, each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, and —CN. In some embodiments, each R³ is independently selected at each occurrence from C₁₋₆ alkyl optionally substituted with one or more substituents independently selected from halogen, —OR¹⁶, —SR¹⁶, —N(R¹⁶)₂, —C(O)R¹⁶, and —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN. In some embodiments, each R³ is independently selected at each occurrence from hydrogen and C₁₋₆ alkyl optionally substituted with one or more substituents independently selected from halogen, —OR¹⁶, —SR¹⁶, —N(R¹⁶)₂, —C(O)R¹⁶, and —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN. In some embodiments, each R³ is independently selected at each occurrence from hydrogen, halogen, —OR¹⁶, —N(R¹⁶)₂, —NO₂, —CN, C₁₋₃ alkyl, and C₁₋₃ haloalkyl. In some embodiments, each R³ is independently selected at each occurrence from hydrogen and methyl. In some embodiments, each R³ is hydrogen.

In some embodiments, for the compound or salt of Formula (III), (IV), (IV-a) and (IV-b), m is 2.

In some embodiments, for the compound or salt of Formula (III), (IV), (IV-a) and (IV-b), m is 1.

In some embodiments, for the compound or salt of Formula (III), (IV), (IV-a) and (IV-b), m is 0.

In some embodiments, for the compound or salt of Formula (III), (IV), (IV-a) and (IV-b), each R⁴ is independently selected from at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN, and C₁₋₆ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —C(O)OR¹⁷, —OC(O)R¹⁷, —S(O)R¹⁷, —S(O)₂R¹⁷, —NO₂, ═O, ═S, ═N(R¹⁷), and —CN. In some embodiments, each R⁴ is independently selected from at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, and —CN. In some embodiments, each R⁴ is independently selected from at each occurrence from C₁₋₆ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —C(O)OR¹⁷, —OC(O)R¹⁷, —S(O)R¹⁷, —S(O)₂R¹⁷, —NO₂, ═O, ═S, ═N(R¹⁷), and —CN. In some embodiments, each R⁴ is selected from halogen, —OR¹⁷, —N(R¹⁷)₂, —NO₂, —CN, C₁₋₃ alkyl, and C₁₋₃ haloalkyl. In some embodiments, each R⁴ is selected from chloro, fluoro, methyl, ethyl, and —CN.

In some embodiments, for the compound or salt of Formula (III), (IV), (IV-a) and (IV-b), A is optionally substituted C₃₋₆ carbocycle. In some embodiments, A is optionally substituted saturated C₃₋₆ carbocycle. In some embodiments, A is optionally substituted unsaturated C₃₋₆ carbocycle. In some embodiments, A is selected from optionally substituted C₃₋₅ carbocycle, optionally substituted C₃₋₄ carbocycle, optionally substituted C₄₋₆ carbocycle, and optionally substituted C₅₋₆ carbocycle. In some embodiments, A is selected from optionally substituted C₃ carbocycle, optionally substituted C₄ carbocycle, optionally substituted C₅ carbocycle, and optionally substituted C₆ carbocycle.

In some embodiments, for the compound or salt of Formula (III), (IV), (IV-a) and (IV-b), A is optionally substituted 5- to 6-membered heteroaryl. In some embodiments, A is optionally substituted 5- to 6-membered heteroaryl comprising at least one heteroatom selected from nitrogen, oxygen, and sulfur. In some embodiments, A is optionally substituted 5- to 6-membered heteroaryl comprising at least one nitrogen or oxygen heteroatom. In some embodiments, A is optionally substituted 5- to 6-membered heteroaryl comprising at least one nitrogen or sulfur heteroatom. In some embodiments, A is optionally substituted 5- to 6-membered heteroayl comprising at least one sulfur or oxygen heteroatom. In some embodiments, A is optionally substituted 5- to 6-membered heteroaryl comprising at least one sulfur heteroatom. In some embodiments, A is optionally substituted 5- to 6-membered heteroaryl comprising at least one oxygen heteroatom. In some embodiments, A is optionally substituted 5- to 6-membered heteroaryl comprising at least one nitrogen heteroatom.

In some embodiments, for the compound or salt of Formula (III), (IV), (IV-a) and (IV-b), A is selected from a saturated C₃₋₆ carbocycle and a 5-membered heteroaryl, any of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹,
—OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein C₃₋₁₀ carbocycle and 3- to 10-membered heterocycle are each optionally substituted with one or more substituents selected from: halogen, —OR¹¹,
—N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN.

In some embodiments, for the compound or salt of Formula (III), (IV), (IV-a) and (IV-b), A is 5- to 6-membered heteroaryl, each of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹,
—OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein C₃₋₁₀ carbocycle and 3- to 10-membered heterocycle are each optionally substituted with one or more substituents selected from: halogen, —OR¹¹,
—N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN.

In some embodiments, for the compound or salt of Formula (III), (IV), (IV-a) and (IV-b), A is 5-membered heteroaryl optionally substituted with one or more substituents independently selected from:
halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN; and
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN, C₃₋₁₀ carbocycle and 3- to 10-membered heterocycle; wherein C₃₋₁₀ carbocycle and 3- to 10-membered heterocycle are each optionally substituted with one or more substituents selected from: —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

In some embodiments, for the compound or salt of Formula (III), (IV), (IV-a) and (IV-b), A is 5- to 6-membered heteroaryl optionally substituted with one or more substituents independently selected from: halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN. In some embodiments, A is 5- to 6-membered heteroaryl optionally substituted with one or more C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN, C₃₋₆ carbocycle and 3- to 6-membered heterocycle; wherein C₃₋₆ carbocycle and 3- to 6-membered heterocycle are each optionally substituted with one or more substituents selected from: —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

In some embodiments, for the compound or salt of Formula (III), (IV), (IV-a) and (IV-b), A is selected from pyrazolyl, imidazolyl, oxazolyl, isoxazolyl, oxadiazolyl, triazolyl, and tetrazolyl, any one of which is optionally substituted with one or more C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN. In some embodiments, A is selected from pyrazolyl and oxadiazolyl, each of which is optionally substituted with C₁₋₆ alkyl optionally substituted with one or more substituents independently selected from halogen, —OR¹¹, —N(R¹¹)₂, —C(O)R¹¹, —NO₂, and —CN. In some embodiments, A is selected from pyrazolyl and oxadiazolyl, each of which is optionally substituted with methyl, ethyl, isopropyl, and or propyl. In some embodiments, A is

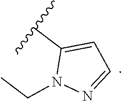 [SMILES: *c1ccnn1CC]

In some embodiments, for the compound or salt of Formula (III), (IV), (IV-a) and (IV-b), A is pyrazolyl optionally substituted with C₁₋₆ alkyl optionally substituted with one or more substituents independently selected from halogen, —OR¹¹, —N(R¹¹)₂, —C(O)R¹¹, —NO₂, and —CN. In some embodiments, A is pyrazolyl optionally substituted with methyl, ethyl, isopropyl, or propyl. In some embodiments, A is N

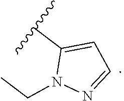 [SMILES: *c1ccnn1CC]

In some embodiments, for the compound or salt of Formula (III), (IV), (IV-a), and (IV-b), A is selected from pyrazolyl, oxazolyl, isoxazolyl, and oxadiazolyl, any of which is optionally substituted with one or more C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN. In some embodiments, A is selected from pyrazolyl, oxazolyl, and isoxazolyl, oxadiazolyl, any of which is optionally substituted with one or more C₁₋₆ alkyl optionally substituted with one or more substituents independently selected from halogen, —OR¹¹, —N(R¹¹)₂, —C(O)R¹¹, —NO₂, and —CN.

In some embodiments, for the compound or salt of Formula (III), (IV), (IV-a), and (IV-b), A is pyrazolyl, oxazolyl, and isoxazolyl, any of which is optionally substituted with one or more C₁₋₆ alkyl and C₁₋₆ haloalkyl. In some embodiments, A is pyrazolyl, oxazolyl, and isoxazolyl, any of which is optionally substituted with one or more methyl, ethyl, isopropyl, propyl, trifluoromethyl, and trifluoroethyl. In some embodiments, A is selected from

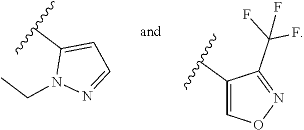

In some embodiments, A is selected from

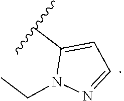 [SMILES: *c1ccnn1CC]

In some embodiments, A is selected from

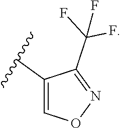 [SMILES: *c1conc1C(F)(F)F]

In some embodiments, for the compound or salt of Formula (III), (IV), (IV-a) and (IV-b), B is —C(H)(R⁵)₂. In some embodiments, each R⁵ is independently selected at each occurrence from halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), and —CN. In some embodiments, each R⁵ is independently selected at each occurrence from C₁₋₆ alkyl optionally substituted with one or more substituents independently selected from halogen, —OR¹³, —SR¹³,
—N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), and —CN. In some embodiments, each R⁵ is independently selected at each occurrence from C₃₋₆ carbocycle optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), and —CN. In some embodiments, each R⁵ is independently selected at each occurrence from C₃₋₆ carbocycle optionally substituted with one or more substituents C₁₋₆ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —S(O)R¹³, —S(O)₂R¹³, —NO₂, ═O, ═S, ═N(R¹³), and —CN.

In some embodiments, for the compound or salt of Formula (III), (IV), (IV-a) and (IV-b), each R⁵ of —C(H)(R⁵)₂ is independently selected from:
halogen, —OR¹³, —N(R¹³)₂, —C(O)R¹³, —C(O)OR¹³, —NO₂, —CN;
C₁₋₆ alkyl optionally substituted with one or more substituents independently selected from halogen, —OR¹³, —N(R¹³)₂, —C(O)R¹³, —C(O)OR¹³, —NO₂, ═O, ═S, ═N(R¹³), and —CN; and
C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl.

In some embodiments, for the compound or salt of Formula (III), (IV), (IV-a) and (IV-b), R⁵ is optionally substituted C₃₋₁₀ carbocycle. In some embodiments, R⁵ is selected from optionally substituted C₃₋₄ carbocycle, optionally substituted C₃₋₅ carbocycle, optionally substituted C₃₋₆ carbocycle, optionally substituted C₃₋₇ carbocycle, optionally substituted C₃₋₈ carbocycle, and optionally substituted C₃₋₉ carbocycle. In some embodiments, R⁵ is selected from optionally substituted C₄₋₁₀ carbocycle, optionally substituted C₅₋₁₀ carbocycle, optionally substituted C₆₋₁₀ carbocycle, optionally substituted C₇₋₁₀ carbocycle, optionally substituted C₈₋₁₀ carbocycle, and optionally substituted C₉₋₁₀ carbocycle. In some embodiments, R⁵ is an optionally substituted C₃₋₁₀ saturated carbocycle. In some embodiments, R⁵ is an optionally substituted C₃₋₁₀ unsaturated carbocycle. In some embodiments, R⁵ is selected from an optionally substituted C₃₋₆ carbocycle and optionally substituted C₆₋₁₀ carbocycle. In some embodiments, R⁵ is selected from an optionally substituted C₃₋₈ monocyclic carbocycle and optionally substituted C₆₋₁₀ bicyclic carbocycle.

In some embodiments, for the compound or salt of Formula (III), (IV), (IV-a) and (IV-b), each R⁵ of —C(H)(R⁵)₂ is independently selected from C₃₋₆ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl. In some embodiments, —CH(R⁵)₂ is

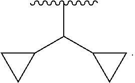 [SMILES: *C(C1CC1)C1CC1]

In some embodiments, for the compound or salt of Formula (III), (IV), (IV-a), and (IV-b), each R⁵ of —C(H)(R⁵)₂ is independently selected from: C₁₋₆ alkyl optionally substituted with one or more substituents independently selected from halogen, —OR¹³, —N(R¹³)₂, —C(O)R¹³, —C(O)OR¹³, —NO₂, ═O, ═S, ═N(R¹³), and —CN; and C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl

In some embodiments, for the compound or salt of Formula (III), (IV), (IV-a) and (IV-b), each R⁵ of —C(H)(R⁵)₂ is independently selected from C₁₋₆ alkyl, C₁₋₆ haloalkyl, C₃₋₇ cycloalkyl, and phenyl, wherein the C₃₋₇ cycloalkyl and phenyl are each optionally substituted with one or more substituents independently selected from halogen, C₁₋₆ alkyl, and C₁₋₆ haloalkyl. In some embodiments, each R⁵ of —C(H)(R⁵)₂ is independently selected from C₁₋₆ alkyl, C₃₋₇ cycloalkyl, and phenyl, wherein the C₃₋₇ cycloalkyl and phenyl are each optionally substituted with one or more substituents independently selected from halogen and C₁₋₆ alkyl. In some embodiments, each R⁵ of —C(H)(R⁵)₂ is independently selected from C₁₋₆ alkyl and C₃₋₇ cycloalkyl. In some embodiments, each R⁵ of —C(H)(R⁵)₂ is C₃₋₇ cycloalkyl. In some embodiments, each R⁵ of —C(H)(R⁵)₂ is independently selected from C₁₋₆ alkyl and phenyl.

In some embodiments, for the compound or salt of Formula (III), (IV), (IV-a) and (IV-b), —C(H)(R⁵)₂ is selected from

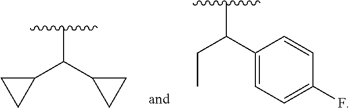 [SMILES: *C(C1CC1)C1CC1 ; *C(CC)c1ccc(F)cc1]

In some embodiments, —C(H)(R⁵)₂ is

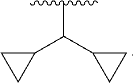 [SMILES: *C(C1CC1)C1CC1]

In some embodiments, —C(H)(R⁵)₂ is

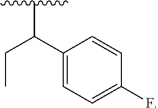 [SMILES: *C(CC)c1ccc(F)cc1]

In some embodiments, for the compound or salt of Formula (III), (IV), (IV-a) and (IV-b), B is selected from

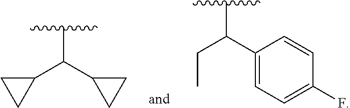 [SMILES: *C(C1CC1)C1CC1 ; *C(CC)c1ccc(F)cc1]

In some embodiments, B is

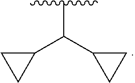 [SMILES: *C(C1CC1)C1CC1]

In some embodiments, B is

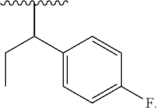 [SMILES: *C(CC)c1ccc(F)cc1]

In some embodiments, for the compound or salt of Formula (III), (IV), (IV-a) and (IV-b), B is optionally substituted C₃₋₁₀ carbocycle. In some embodiments, B is selected from optionally substituted C₃₋₄ carbocycle, optionally substituted C₃₋₅ carbocycle, optionally substituted C₃₋₆ carbocycle, optionally substituted C₃₋₇ carbocycle, optionally substituted C₃₋₈ carbocycle, and optionally substituted C₃₋₉ carbocycle. In some embodiments, B is selected from optionally substituted C₄₋₁₀ carbocycle, optionally substituted C₅₋₁₀ carbocycle, optionally substituted C₆₋₁₀ carbocycle, optionally substituted C₇₋₁₀ carbocycle, optionally substituted C₈₋₁₀ carbocycle, and optionally substituted C₉₋₁₀ carbocycle. In some embodiments, B is an optionally substituted C₃₋₁₀ saturated carbocycle. In some embodiments, B is an optionally substituted C₃₋₁₀ unsaturated carbocycle. In some embodiments, B is selected from an optionally substituted C₃₋₆ carbocycle and optionally substituted C₆₋₁₀ carbocycle. In some embodiments, B is selected from an optionally substituted C₃₋₈ monocyclic carbocycle and optionally substituted C₆₋₁₀ bicyclic carbocycle.

In some embodiments, for the compound or salt of Formula (III), (IV), (IV-a) and (IV-b), B is C₆₋₁₀ carbocycle optionally substituted with one or more substituents independently selected from: halogen, —OR¹², N(R¹²)₂, —C(O)R¹², —NO₂, ═O, ═S, ═N(R¹²), —CN, and C₁₋₆ alkyl optionally substituted with one or more substituents independently selected from halogen, —OR¹², N(R¹²)₂, —C(O)R¹², —NO₂, ═O, ═S, ═N(R¹²), and —CN. In some embodiments, B is C₆₋₁₀ carbocycle optionally substituted with one or more substituents independently selected from: halogen, —OR¹², N(R¹²)₂, —C(O)R¹², —NO₂, ═O, ═S, ═N(R¹²), —CN. In some embodiments, B is C₆₋₁₀ carbocycle optionally substituted with one or more C₁₋₆ alkyl optionally substituted with one or more substituents independently selected from halogen, —OR¹², N(R¹²)₂, —C(O)R¹², —NO₂, ═O, ═S, ═N(R¹²), and —CN.

In some embodiments, for the compound or salt of Formula (III), (IV), (IV-a) and (IV-b), B is selected from cyclohexyl and cycloheptyl, each of which is optionally substituted with one or more substituents independently selected from: halogen, —OR¹², N(R¹²)₂, —NO₂, —CN, and C₁₋₆ alkyl optionally substituted with one or more substituents independently selected from halogen, —OR¹², N(R¹²)₂, —C(O)R¹², —NO₂, ═O, ═S, ═N(R¹²), and —CN. In some embodiments, B is selected from cyclohexyl and cycloheptyl, each of which is optionally substituted with one or more substituents independently selected from halogen, C₁₋₆ alkyl, and C₁₋₆ haloalkyl. In some embodiments, B is

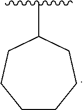 [SMILES: *C1CCCCCC1]

In some embodiments, B is selected from

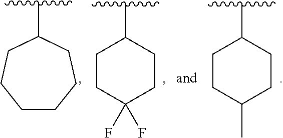

In some embodiments, B is

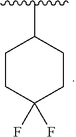 [SMILES: *C1CCC(F)(F)CC1]

In some embodiments, B is

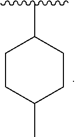 [SMILES: *C1CCC(C)CC1]

In some aspects, the present disclosure provides a compound of Formula (III) selected from:

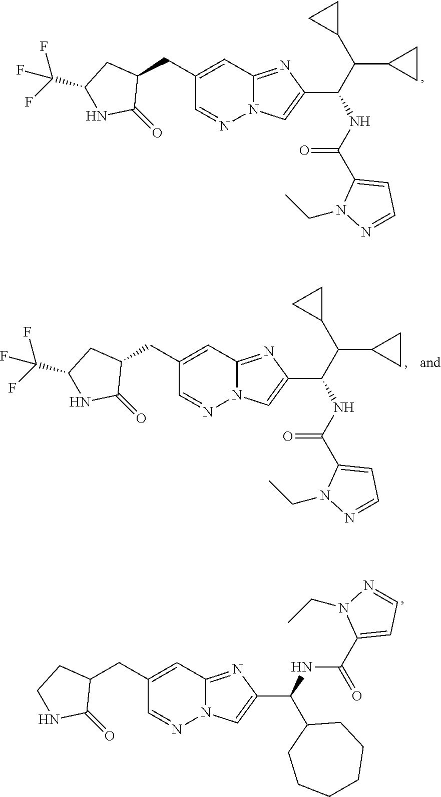 [SMILES: CCn1nccc1C(=O)N[C@H](c1cn2ncc(C[C@@H]3C[C@@H](C(F)(F)F)NC3=O)cc2n1)C(C1CC1)C1CC1 ; CCn1nccc1C(=O)N[C@H](c1cn2ncc(C[C@H]3C[C@@H](C(F)(F)F)NC3=O)cc2n1)C(C1CC1)C1CC1 ; CCn1nccc1C(=O)N[C@H](c1cn2ncc(CC3CCNC3=O)cc2n1)C1CCCCCC1]

or pharmaceutically acceptable salts thereof.

In some aspects, the present disclosure provides a compound of Formula (III) selected from

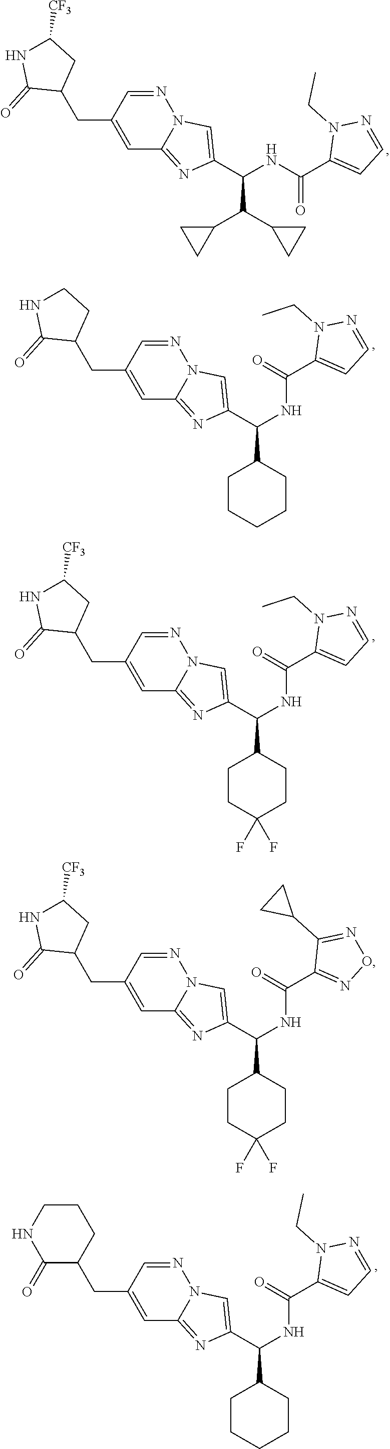

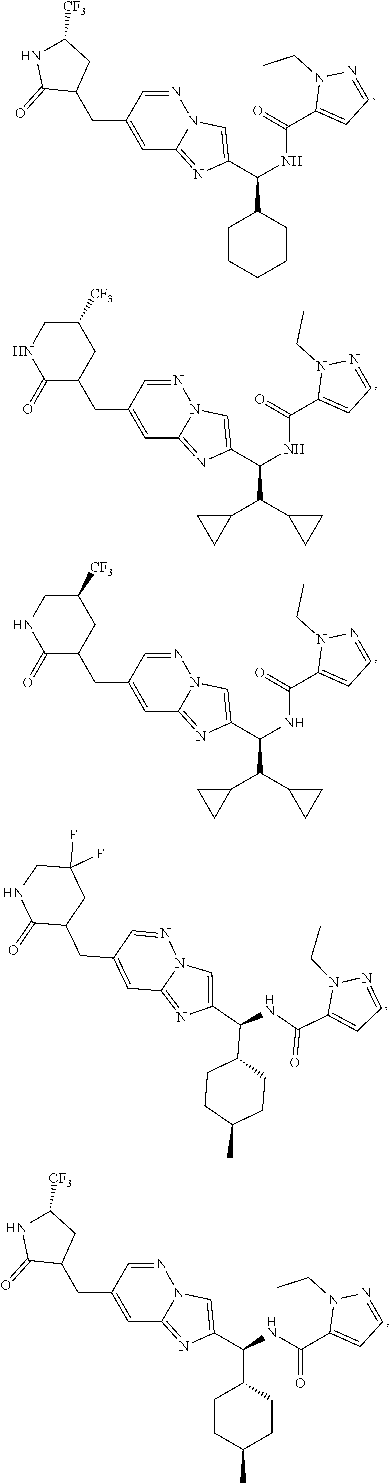

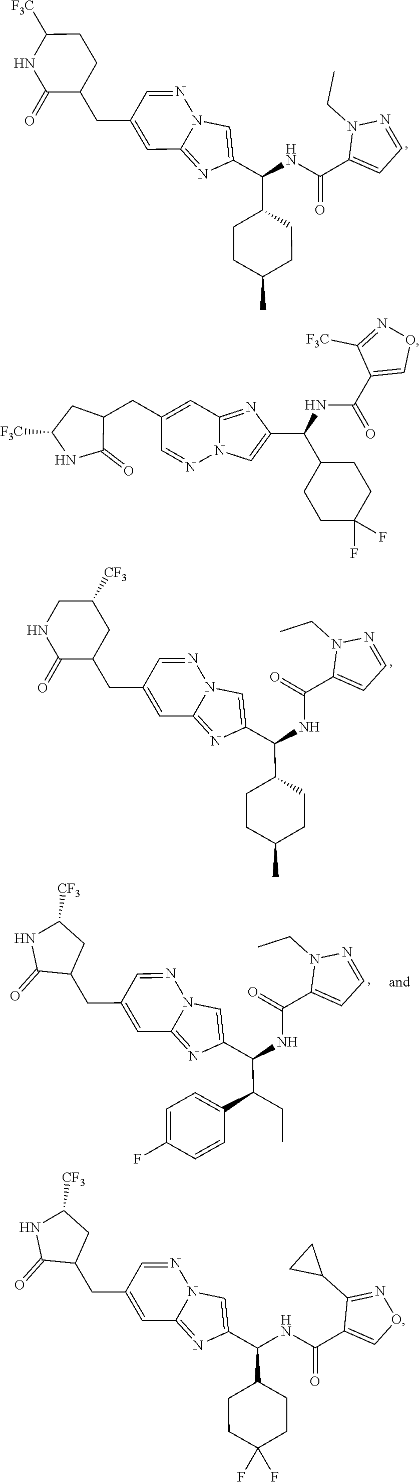 [SMILES: CCn1nccc1C(=O)N[C@H](c1cn2ncc(CC3CCC(C(F)(F)F)NC3=O)cc2n1)[C@H]1CC[C@H](C)CC1 ; O=C(N[C@H](c1cn2ncc(CC3C[C@@H](C(F)(F)F)NC3=O)cc2n1)C1CCC(F)(F)CC1)c1conc1C(F)(F)F ; CCn1nccc1C(=O)N[C@H](c1cn2ncc(CC3C[C@@H](C(F)(F)F)CNC3=O)cc2n1)[C@H]1CC[C@H](C)CC1 ; CC[C@@H](c1ccc(F)cc1)[C@H](NC(=O)c1ccnn1CC)c1cn2ncc(CC3C[C@@H](C(F)(F)F)NC3=O)cc2n1 ; O=C(N[C@H](c1cn2ncc(CC3C[C@@H](C(F)(F)F)NC3=O)cc2n1)C1CCC(F)(F)CC1)c1conc1C1CC1]

or pharmaceutically acceptable salts thereof.

In certain aspects, the present disclosure provides a compound or salt represented by the structure of Formula (III) wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)OR¹¹, —NO₂, —CN;
   C₁₋₁₀ alkyl optionally substituted with one or more substituents independently selected halogen, —OR¹¹, —SR¹¹, —N(R¹¹)₂, —C(O)R¹¹, —C(O)OR¹¹, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —SR¹¹, —N(R¹¹)₂, —C(O)R¹¹, —C(O)OR¹¹, —NO₂, and —CN; and
   C₃₋₁₀ carbocycle optionally substituted with one or more substituents independently selected from halogen, —OR^{11A}, —SR^{11A}, —N(R^{11A})₂, —C(O)R^{1A}, —C(O)OR^{11A}, —NO₂, —CN; and C₁₋₁₀ alkyl optionally substituted with one or more substituents selected from: halogen, —OR^{11A}, —SR^{11A}, —N(R^{11A})₂, —C(O)R^{11A}, —C(O)OR^{11A}, —NO₂, and —CN, for example, A is 5-membered heteroaryl optionally substituted with C₃₋₁₀ carbocycle;
B is selected from —C(H)(R⁵)₂ and C₃₋₁₀ carbocycle, wherein each C₃₋₁₀ carbocycle is optionally substituted with one or more substituents independently selected from:
   halogen, —OR¹², —SR¹², —N(R¹²)₂, —C(O)R¹², —C(O)OR¹², —NO₂, —CN;
   C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)OR¹², —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —SR¹², —N(R¹²)₂, —C(O)R¹², —C(O)OR¹², —NO₂, and —CN, for example B is optionally substituted C₃₋₁₀ carbocycle;
each R⁵ is independently selected at each occurrence from (i), (ii), and (iii):
   (i) halogen, —OR¹³, —SR¹³, —N(R¹³)₂, —C(O)R¹³, —C(O)OR¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN; and
   (iii) C₃₋₁₀ carbocycle optionally substituted with one or more substituents independently selected from: halogen, —OR¹³, —SR¹³, —N(R¹³)₂, —C(O)R¹³, —C(O)OR¹³, —NO₂, —CN, and C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN,
   for example, each R⁵ is optionally substituted C₃₋₁₀ carbocycle or optionally substituted C₁₋₁₀ alkyl;
each R¹ is independently selected at each occurrence from:
   halogen, —OR¹⁴, —SR¹⁴, —N(R¹⁴)₂, —C(O)R¹⁴, —C(O)OR¹⁴, —NO₂, —CN;
   C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN; and
   C₃₋₁₀ carbocycle optionally substituted with one or more substituents independently selected from halogen, —OR¹⁴, —SR¹⁴, —N(R¹⁴)₂, —C(O)R¹⁴, —C(O)OR¹⁴, —NO₂, —CN; and C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN, for example, each R¹ is C₁₋₁₀ haloalkyl;
R² is selected from:
   hydrogen, halogen, —OR¹⁵, —SR¹⁵, —N(R¹⁵)₂, —C(O)R¹⁵, —C(O)OR¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)OR¹⁵, —NO₂, and —CN, for example R² is hydrogen;
each R³ is independently selected at each occurrence from:
   hydrogen, halogen, —OR¹⁶, —SR¹⁶, —N(R¹⁶)₂, —C(O)R¹⁶, —C(O)OR¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)OR¹⁶, —NO₂, and —CN, for example each R³ hydrogen or optionally substituted C₁₋₆ alkyl;
each R⁴ is independently selected at each occurrence from
   halogen, —OR¹⁷, —SR¹⁷, —N(R¹⁷)₂, —C(O)R¹⁷, —C(O)OR¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)OR¹⁷, —NO₂, and —CN, for example each R⁴ is halogen or optionally substituted C₁₋₁₀ alkyl;
R¹¹, R^{11A}, R¹², R¹³, R¹⁴, R¹⁵, R¹⁶, and R¹⁷ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2, for example p is 1;
m is selected from 0 and 1, for example m is 0; and
p is selected from 0, 1, and 2, for example p is 1.

In certain aspects, the present disclosure provides a compound or salt represented by the structure of Formula (III) wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)OR¹¹, —NO₂, —CN;
   C₁₋₁₀ alkyl optionally substituted with one or more substituents independently selected halogen, —OR¹¹, —SR¹¹, —N(R¹¹)₂, —C(O)R¹¹, —C(O)OR¹¹, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —SR¹¹, —N(R¹¹)₂, —C(O)R¹¹, —C(O)OR¹¹, —NO₂, and —CN; and
   C₃₋₁₀ carbocycle optionally substituted with one or more substituents independently selected from halogen, —OR^{11A}, —SR^{11A}, —N(R^{11A})₂, —C(O)R^{11A}, —C(O)OR^{11A}, —NO₂, —CN; and C₁₋₁₀ alkyl optionally substituted with one or more substituents selected from: halogen, —OR^{11A}, —SR^{11A}, —N(R^{11A})₂, —C(O)R^{11A}, —C(O)OR^{11A}, —NO₂, and —CN, for example A is 5-membered heteroaryl optionally substituted with C₃₋₁₀ carbocycle;
B is —C(H)(R⁵)₂ and each R⁵ is independently selected at each occurrence from (i), (ii), and (iii):
   (i) halogen, —OR¹³, —SR¹³, —N(R¹³)₂, —C(O)R¹³, —C(O)OR¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN; and
   (iii) C₃₋₁₀ carbocycle optionally substituted with one or more substituents independently selected from: halogen, —OR¹³, —SR¹³, —N(R¹³)₂, —C(O)R¹³, —C(O)OR¹³, —NO₂, —CN
      C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN,
   for example, each R⁵ is selected from optionally substituted C₃₋₁₀ carbocycle or optionally substituted C₁₋₁₀ alkyl;
each R¹ is independently selected at each occurrence from:
   halogen, —OR¹⁴, —SR¹⁴, —N(R¹⁴)₂, —C(O)R¹⁴, —C(O)OR¹⁴, —NO₂, —CN;
   C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN; and
   C₃₋₁₀ carbocycle optionally substituted with one or more substituents independently selected from halogen, —OR¹⁴, —SR¹⁴, —N(R¹⁴)₂, —C(O)R¹⁴, —C(O)OR¹⁴, —NO₂, —CN; and C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN, for example, each R¹ is C₁₋₁₀ haloalkyl;
R² is selected from:
   hydrogen, halogen, —OR¹⁵, —SR¹⁵, —N(R¹⁵)₂, —C(O)R¹⁵, —C(O)OR¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)OR¹⁵, —NO₂, and —CN, for example R² is hydrogen;
each R³ is independently selected at each occurrence from:
   hydrogen, halogen, —OR¹⁶, —SR¹⁶, —N(R¹⁶)₂, —C(O)R¹⁶, —C(O)OR¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)OR¹⁶, —NO₂, and —CN, for example each R³ hydrogen or optionally substituted C₁₋₆ alkyl;
each R⁴ is independently selected at each occurrence from
   halogen, —OR¹⁷, —SR¹⁷, —N(R¹⁷)₂, —C(O)R¹⁷, —C(O)OR¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)OR¹⁷, —NO₂, and —CN, for example each R⁴ is halogen or optionally substituted C₁₋₁₀ alkyl;
R¹¹, R^{11A}, R¹², R¹³, R¹⁴, R¹⁵, R¹⁶, and R¹⁷ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2, for example p is 1;
m is selected from 0 and 1, for example m is 0; and
p is selected from 0, 1, and 2, for example p is 1.

In certain aspects, the present disclosure provides a compound or salt represented by the structure of Formula (III), (IV), (IV-a), or (IV-b), wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)OR¹¹, —NO₂, —CN; and
   C₁₋₁₀ alkyl optionally substituted with one or more substituents independently selected halogen, —OR¹¹, —SR¹¹, —N(R¹¹)₂, —C(O)R¹¹, —C(O)OR¹¹, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —SR¹¹, —N(R¹¹)₂, —C(O)R¹¹, —C(O)OR¹¹, —NO₂, and —CN, for example, A is 5-membered heteroaryl optionally substituted with C₁₋₁₀alkyl;
B is selected from —C(H)(R⁵)₂ and C₃₋₁₀ carbocycle, wherein each C₃₋₁₀ carbocycle is optionally substituted with one or more substituents independently selected from:
   halogen, —OR¹², —SR¹², —N(R¹²)₂, —C(O)R¹², —C(O)OR¹², —NO₂, —CN;
   C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)OR¹², —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —SR¹², —N(R¹²)₂, —C(O)R¹², —C(O)OR¹², —NO₂, and —CN, for example B is optionally substituted C₃₋₁₀ carbocycle;
each R⁵ is independently selected at each occurrence from (i), (ii), and (iii):
   (i) halogen, —OR¹³, —SR¹³, —N(R¹³)₂, —C(O)R¹³, —C(O)OR¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN; and
   (iii) C₃₋₁₀ carbocycle optionally substituted with one or more substituents independently selected from: halogen, —OR¹³, —SR¹³, —N(R¹³)₂, —C(O)R¹³, —C(O)OR¹³, —NO₂, —CN, and C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN,
   for example, each R⁵ is optionally substituted C₃₋₁₀ carbocycle or optionally substituted C₁₋₁₀ alkyl;
each R¹ is independently selected at each occurrence from:
   halogen, —OR¹⁴, —SR¹⁴, —N(R¹⁴)₂, —C(O)R¹⁴, —C(O)OR¹⁴, —NO₂, —CN;
   C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN; and
   C₃₋₁₀ carbocycle optionally substituted with one or more substituents independently selected from halogen, —OR¹⁴, —SR¹⁴, —N(R¹⁴)₂, —C(O)R¹⁴, —C(O)OR¹⁴, —NO₂, —CN; and C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN, for example, each R¹ is C₁₋₁₀ haloalkyl;
R² is selected from:
   hydrogen, halogen, —OR¹⁵, —SR¹⁵, —N(R¹⁵)₂, —C(O)R¹⁵, —C(O)OR¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)OR¹⁵, —NO₂, and —CN, for example R² is hydrogen;
each R³ is independently selected at each occurrence from:
   hydrogen, halogen, —OR¹⁶, —SR¹⁶, —N(R¹⁶)₂, —C(O)R¹⁶, —C(O)OR¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)OR¹⁶, —NO₂, and —CN, for example each R³ hydrogen or optionally substituted C₁₋₆ alkyl;
each R⁴ is independently selected at each occurrence from
   halogen, —OR¹⁷, —SR¹⁷, —N(R¹⁷)₂, —C(O)R¹⁷, —C(O)OR¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)OR¹⁷, —NO₂, and —CN, for example each R⁴ is halogen or optionally substituted C₁₋₁₀ alkyl;
R¹¹, R¹², R¹³, R¹⁴, R¹⁵, R¹⁶, and R¹⁷ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2, for example p is 1;
m is selected from 0 and 1, for example m is 0; and
p is selected from 0, 1, and 2, for example p is 1.

In certain aspects, the present disclosure provides a compound or salt represented by the structure of Formula (III), (IV), (IV-a), or (IV-b), wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)OR¹¹, —NO₂, —CN;
   C₁₋₁₀ alkyl optionally substituted with one or more substituents independently selected halogen, —OR¹¹, —SR¹¹, —N(R¹¹)₂, —C(O)R¹¹, —C(O)OR¹¹, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —SR¹¹, —N(R¹¹)₂, —C(O)R¹¹, —C(O)OR¹¹, —NO₂, and —CN; and for example A is 5-membered heteroaryl optionally substituted with C₁₋₁₀ alkyl;
B is —C(H)(R⁵)₂ and each R⁵ is independently selected at each occurrence from (i), (ii), and (iii):
   (i) halogen, —OR¹³, —SR¹³, —N(R¹³)₂, —C(O)R¹³, —C(O)OR¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN; and
   (iii) C₃₋₁₀ carbocycle optionally substituted with one or more substituents independently selected from: halogen, —OR¹³, —SR¹³, —N(R¹³)₂, —C(O)R¹³, —C(O)OR¹³, —NO₂, —CN
      C₁₋₁₀ alkyl optionally substituted with one or more substituents independently selected from halogen, —OR¹³, —SR¹³, —N(R¹³)₂, —C(O)R¹³, —C(O)OR¹³, —NO₂, and —CN,
   for example, each R⁵ is selected from optionally substituted C₃₋₁₀ carbocycle or optionally substituted C₁₋₁₀ alkyl;
each R¹ is independently selected at each occurrence from:
   halogen, —OR¹⁴, —SR¹⁴, —N(R¹⁴)₂, —C(O)R¹⁴, —C(O)OR¹⁴, —NO₂, —CN;
   C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN; and
   C₃₋₁₀ carbocycle optionally substituted with one or more substituents independently selected from halogen, —OR¹⁴, —SR¹⁴, —N(R¹⁴)₂, —C(O)R¹⁴, —C(O)OR¹⁴, —NO₂, —CN; and C₁₋₁₀ alkyl optionally substituted with one or more substituents independently selected from halogen, —OR¹⁴, —SR¹⁴, —N(R¹⁴)₂, —C(O)R¹⁴, —C(O)OR¹⁴, —NO₂, and —CN, for example, each R¹ is C₁₋₁₀ haloalkyl;
R² is selected from:
   hydrogen, halogen, —OR¹⁵, —SR¹⁵, —N(R¹⁵)₂, —C(O)R¹⁵, —C(O)OR¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)OR¹⁵, —NO₂, and —CN, for example R² is hydrogen;
each R³ is independently selected at each occurrence from:
   hydrogen, halogen, —OR¹⁶, —SR¹⁶, —N(R¹⁶)₂, —C(O)R¹⁶, —C(O)OR¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)OR¹⁶, —NO₂, and —CN, for example each R³ hydrogen or optionally substituted C₁₋₆ alkyl;
each R⁴ is independently selected at each occurrence from
   halogen, —OR¹⁷, —SR¹⁷, —N(R¹⁷)₂, —C(O)R¹⁷, —C(O)OR¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)OR¹⁷, —NO₂, and —CN, for example each R⁴ is halogen or optionally substituted C₁₋₁₀ alkyl;
R¹¹, R¹², R¹³, R¹⁴, R¹⁵, R¹⁶, and R¹⁷ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2, for example p is 1;
m is selected from 0 and 1, for example m is 0; and
p is selected from 0, 1, and 2, for example p is 1.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds or salts of Formula (I), (II), (II-a), (II-b), (III), (IV), (IV-a), or (IV-b), are intended to include all Z-, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-PrelogR-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)— or (S)—. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The compounds or salts for Formula (I), (II), (II-a), (II-b), (III), (IV), (IV-a), or (IV-b), may in some cases exist as diastereomers, enantiomers, or other stereoisomeric forms. The compounds presented herein include all diastereomeric, enantiomeric, and epimeric forms as well as the racemates, mixtures of diastereomers, and other mixtures thereof, to the extent they can be made by one of ordinary skill in the art by routine experimentation. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis. Furthermore, a mixture of two enantiomers enriched in one of the two can be purified to provide further optically enriched form of the major enantiomer by recrystallization and/or trituration.

In certain embodiments, compounds or salts for Formula (I), (II), (II-a), (II-b), (III), (IV), (IV-a), or (IV-b), may comprise two or more enantiomers or diastereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. In certain embodiments, the compounds or salts for Formula (I), (II), (II-a), (II-b), (III), (IV), (IV-a), or (IV-b), exist as tautomers. In circumstances where tautomerization is possible, a chemical equilibrium of the tautomers may exist. The exact ratio of the tautomers depends on several factors, including physical state, temperature, solvent, and pH. Some non-limiting examples of tautomeric equilibrium include:

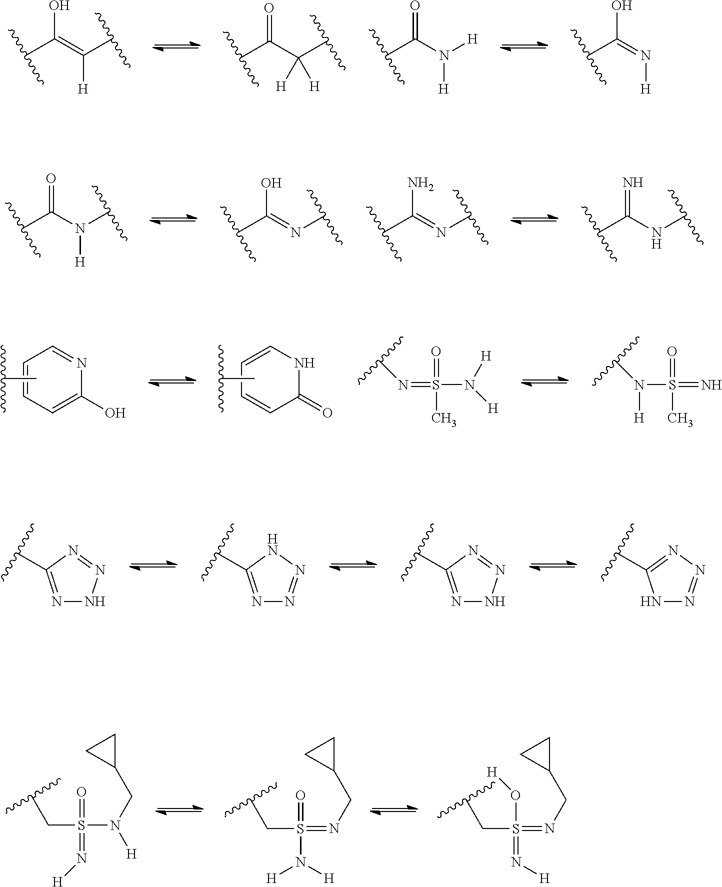

The compounds of Formula (I), (II), (II-a), (II-b), (III), (IV), (IV-a), or (IV-b), can be used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds of Formula (I), (II), (II-a), (II-b), (III), (IV), (IV-a), or (IV-b), have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Unless otherwise stated, compounds of Formula (I), (II), (II-a), (II-b), (III), (IV), (IV-a), or (IV-b), are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of Formula (I), (II), (II-a), (II-b), (III), (IV), (IV-a), or (IV-b), optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formula (I), (II), (II-a), (II-b), (III), (IV), (IV-a), or (IV-b). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride.

In certain embodiments, compounds or salts of Formula (I), (II), (II-a), (II-b), (III), (IV), (IV-a), or (IV-b), may be prodrugs. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Pharmaceutical Formulations

In some aspects, the present disclosure provides a pharmaceutical composition comprising a compound or salt of Formula (I), (II), (II-a), (II-b), (III), (IV), (IV-a), or (IV-b), and at least one pharmaceutically acceptable excipient. In some embodiments, the pharmaceutical composition comprises a compound or salt of Formula (I), (II), (II-a), (II-b), (III), (IV), (IV-a), or (IV-b), and a pharmaceutically acceptable excipient.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically-acceptable salt form.

Pharmaceutical compositions as often further can comprise more than one active compound (e.g., a compound, salt or conjugate and other agents) as necessary for the particular indication being treated. The active compounds can have complementary activities that do not adversely affect each other. Such molecules can be present in combination in amounts that are effective for the purpose intended.

A compound or salt of any one of Formula (I), (II), (II-a), (II-b), (III), (IV), (IV-a), or (IV-b), may be formulated in any suitable pharmaceutical formulation. A pharmaceutical formulation of the present disclosure typically contains an active ingredient (e.g., compound or salt of any one Formula I) and one or more pharmaceutically acceptable excipients or carriers, including but not limited to: inert solid diluents and fillers, diluents, sterile aqueous solution and various organic solvents, permeation enhancers, antioxidents, solubilizers, and adjuvants.

In certain embodiments, a compound or salt of Formula (I), (II), (II-a), (II-b), (III), (IV), (IV-a), or (IV-b), is formulated with a chelating agent or other material capable of binding metal ions, such as ethylene diamine tetra acetic acid (EDTA) and its salts are capable of enhancing the stability of a compound or salt of Formula (I), (II), (II-a), (II-b), (III), (IV), (IV-a), or (IV-b).

Pharmaceutical formulations may be provided in any suitable form, which may depend on the route of administration.

In some embodiments, the disclosure provides a pharmaceutical composition for oral administration containing at least one compound or salt of any one of Formula (I), (II), (II-a), (II-b), (III), (IV), (IV-a), or (IV-b), and a pharmaceutical excipient suitable for oral administration. The composition may be in the form of a solid, liquid, gel, semi-liquid, or semi-solid. In some embodiments, the composition further comprises a second agent.

Pharmaceutical compositions of the disclosure suitable for oral administration can be presented as discrete dosage forms, such as hard or soft capsules, cachets, troches, lozenges, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, or dispersible powders or granules, or syrups or elixirs. Such dosage forms can be prepared by any of the methods of pharmacy, which typically include the step of bringing the active ingredient(s) into association with the carrier. In general, the composition are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient(s) in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound or salt of any one of Formula (I), (II), (II-a), (II-b), (III), (IV), (IV-a), or (IV-b), moistened with an inert liquid diluent.

Pharmaceutical compositions may also be prepared from a compound or salt of any one of Formula (I), (II), (II-a), (II-b), (III), (IV), (IV-a), or (IV-b), and one or more pharmaceutically acceptable excipients. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

### Methods of Treatment

In some aspects, the present disclosure provides a method of modulating IL-17 A in a subject in need thereof, comprising administering to the subject a compound or salt of Formula (I), (II), (II-a), (II-b), (III), (IV), (IV-a), or (IV-b), or a pharmaceutical composition thereof.

Increased levels of IL-17A have been associated with several conditions including airway inflammation, rheumatoid arthritis (RA), osteoarthritis, bone erosion, intraperitoneal abscesses and adhesions, inflammatory bowel disorder (IBD), allograft rejection, psoriasis, psoriatic arthritis, ankylosing spondylitis, certain types of cancer, angiogenesis, atherosclerosis and multiple sclerosis (MS). Both IL-17A and IL-17R are upregulated in the synovial tissue of RA patients. IL-17A exerts its role in pathogenesis of RA through IL-1-β and TNF-α dependent and independent pathways. IL-17A stimulates secretion of other cytokines and chemokines, e.g., TNF-α, IL-1β, IL-6, IL-8 and Gro-α. IL-17A directly contributes to disease progression in RA. Injection of IL-17A into the mouse knee promotes joint destruction independently of IL-I β activity (Ann Rheum Dis 2000, 59:529-32). Anti-IL-1β antibody has no effect on IL-17A induced inflammation and joint damage (J. Immunol 2001, 167:1004-1013). In a streptococcal cell wall (SCW)-induced murine arthritis model, IL-17A induced inflammatory cell infiltration and proteoglycan depletion in wild-type and IL-1β knockout and TNF-α knockout mice. IL-17A knockout mice are phenotypically normal in the absence of antigenic challenge but have markedly reduced arthritis following type II collagen immunization (J. Immunol 2003, 171:6173-6177). Increased levels of IL-17A-secreting cells have also been observed in the facet joints of patients suffering from ankylosing spondylitis (H Appel et al., Arthritis Res Therap. 2011, 13:R95).

Multiple sclerosis is an autoimmune disease characterized by central nervous system (CNS) inflammation with damage to the myelin sheath surrounding axons. A hallmark of MS is that T cells infiltrate into the CNS. Higher numbers of IL-17A mRNA-expressing blood mono-nuclear cells (MNC) are detected during MS clinical exacerbation compared to remission (Multiple Sclerosis, 5:101-104, 1999). Furthermore, experimental autoimmune encephalomyelitis (“EAE”), a preclinical animal model for MS is significantly suppressed in IL-17A knockout mice.

In certain aspects, the disclosure provides methods of modulating IL-17A in a subject in need thereof, comprising administering to said subject a compound or salt of Formula (I), (II), (II-a), (II-b), (III), (IV), (IV-a), or (IV-b). In certain embodiments, a compound or salt of Formula (I), (II), (II-a), (II-b), (III), (IV), (IV-a), or (IV-b), inhibits the activity of IL-17A in a subject in need thereof.

In certain embodiments, a compound or salt of Formula (I), (II), (II-a), (II-b), (III), (IV), (IV-a), or (IV-b), can be used to treat or prevent a disease or condition that is mediated directly or indirectly by IL-17A. Such diseases include inflammatory diseases and conditions, proliferative diseases (e.g., cancer), autoimmune diseases and other disease described herein. The methods generally involve administering therapeutically effective amounts of compounds disclosed herein or a pharmaceutical composition thereof to the subject. In some embodiments, the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, palmoplantar psoriasis, spondyloarthritis, and Non-infectious Uveitis.

In some aspects, the present disclosure provides a method of a method of treating or preventing an inflammatory disease or condition in a subject in need thereof, comprising administering to the subject a compound or salt of Formula (I), (II), (II-a), (II-b), (III), (IV), (IV-a), or (IV-b), or a pharmaceutical composition thereof. In certain embodiments, a compound or salt of Formula (I), (II), (II-a), (II-b), (III), (IV), (IV-a), or (IV-b), is administered to a subject in need thereof to treat an inflammatory disease or condition, e.g., psoriasis.

In certain embodiments, a compound or salt of Formula (I), (II), (II-a), (II-b), (III), (IV), (IV-a), or (IV-b), is used to treat or prevent an inflammatory disease or condition is selected from, plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, Palmoplantar Psoriasis, Spondyloarthritis, and Non-infectious Uveitis. In certain embodiments, a compound or salt of Formula (I), (II), (II-a), (II-b), (III), (IV), (IV-a), or (IV-b), is used to treat or prevent psoriasis. In certain embodiments, a compound or salt of Formula (I), (II), (II-a), (II-b), (III), (IV), (IV-a), or (IV-b), is used for the treatment or prevention of a condition including, but not limited to, airway inflammation, ankylosing spondylitis, asthma, RA (including juvenile RA), as well as other inflammatory disorders, conditions, or diseases.

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Examples 1-209 show exemplary procedures for the preparation of the claimed IL-17A modulators. Example 210 provides IL-17 A/A bioassay IC₅₀ inhibition data. Abbreviations as used in the examples are as follows: DCM=dichloromethane; MeOH=methanol; ACN=acetonitrile; THF=tetrahydrofuran; EtOAc=ethylacetate; PE=petroleum ether; DMF=dimethylformamide; DMSO=dimethyl sulfoxide; DIEA=diisopropylethylamine; LDA=lithium diisopropylamide; FA=formic acid; TFA=trifluoroacetic acid; iPrOH=isopropyl alcohol; NBS=N-bromosuccinimide.

### Example 1: Preparation of Intermediate 1

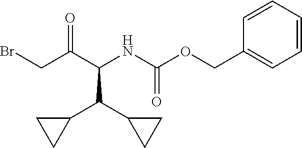 [SMILES: O=C(N[C@H](C(=O)CBr)C(C1CC1)C1CC1)OCc1ccccc1]

### Benzyl (S)-(4-bromo-1,1-dicyclopropyl-3-oxobutan-2-yl)carbamate. (Intermediate 1, Int. 1)

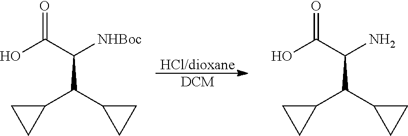 [SMILES: CC(C)(C)OC(=O)N[C@H](C(=O)O)C(C1CC1)C1CC1 ; N[C@H](C(=O)O)C(C1CC1)C1CC1]

A solution of (S)-2-((tert-butoxycarbonyl)amino)-3,3-dicyclopropylpropanoic acid (15.0 g, 55.6 mmol, 1.00 eq) in DCM (150 mL) and HCl in 1,4-dioxane (4 M, 25.0 mL, 1.80 eq) was stirred at 20° C. for 2 h. The reaction mixture was concentrated under reduced pressure to afford (S)-2-amino-3,3-dicyclopropylpropanoic acid (11.0 g, 53.4 mmol, 96% yield, HCl salt) as a white solid. LCMS [M+H]⁺=170.1 m/z.

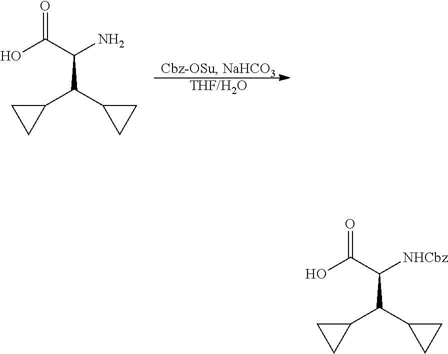 [SMILES: N[C@H](C(=O)O)C(C1CC1)C1CC1 ; O=C(N[C@H](C(=O)O)C(C1CC1)C1CC1)OCc1ccccc1]

To a solution of (S)-2-amino-3,3-dicyclopropylpropanoic acid (11.0 g, 53.4 mmol, 1.00 eq, HCl salt) in THE (100 mL) and water (50.0 mL) was added benzyl (2,5-dioxopyrrolidin-1-yl) carbonate (26.7 g, 106 mmol, 2.00 eq) and NaCO₃ (13.5 g, 160 mmol, 6.24 mL, 3.00 eq). The reaction mixture was stirred at RT for 2 h. The reaction mixture was diluted with a 1M aqueous solution of HCl to pH=4 and extracted with EtOAc, dried over anhydrous Na₂SO₄, filtered and concentrated under reduced pressure to give a residue which was purified by column chromatography (SiO₂, PE/EtOAc) to afford (S)-2-(((benzyloxy)carbonyl)amino)-3,3-dicyclopropylpropanoic acid (10.0 g, 32.9 mmol, 62% yield) as a white solid. ¹H NMR (400 MHz, CDCl₃) δ 7.41-7.30 (m, 5H), 5.57 (d, J=9.2 Hz, 1H), 5.14 (s, 2H), 4.73-4.47 (m, 1H), 0.88-0.67 (m, 3H), 0.59-0.35 (m, 4H), 0.32-0.13 (m, 4H). LCMS [M+H]⁺=304.2 m/z.

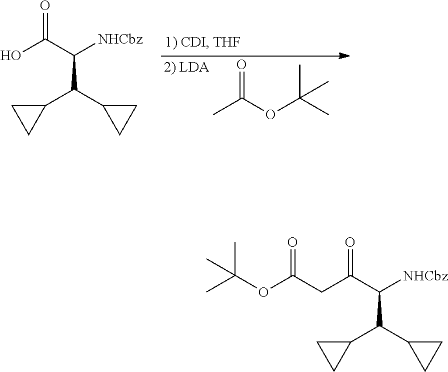

A solution of (S)-2-(((benzyloxy)carbonyl)amino)-3,3-dicyclopropylpropanoic acid (20.0 g, 65.9 mmol, 1.00 eq) in THF (250 mL) and 1,1′-carbonyldiimidazole (11.8 g, 72.5 mmol, 1.10 eq) was stirred at 0° C. for 2 h. Meanwhile, to a solution of tert-butyl acetate (23.0 g, 198 mmol, 26.5 mL, 3.00 eq) in THE (250 mL) was added a solution of LDA (2 M, 98.9 mL, 3.00 eq), and the reaction mixture was stirred at −78° C. for 2 h. The two reaction mixtures were combined and stirred at −78° C. for 2 h. The reaction mixture was diluted with a saturated aqueous solution of NH₄C₁, extracted with EtOAc, dried over anhydrous Na₂SO₄, filtered and concentrated under reduced pressure to give a residue, which was purified by column chromatography (SiO₂, PE/EtOAc) to afford tert-butyl (S)-4-(((benzyloxy)carbonyl)amino)-5,5-dicyclopropyl-3-oxopentanoate (15.0 g, 37.3 mmol, 57% yield) as a yellow oil. LCMS [M+Na]⁺=424.3 m/z.

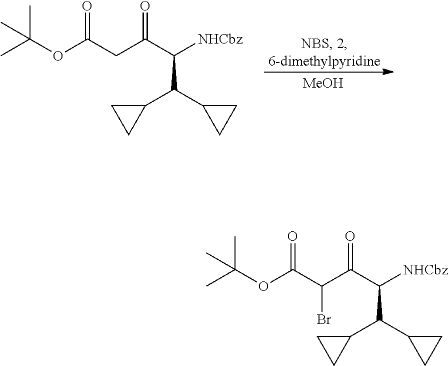 [SMILES: "MeOH" ; CC(C)(C)OC(=O)CC(=O)[C@@H](NC(=O)OCc1ccccc1)C(C1CC1)C1CC1 ; CC(C)(C)OC(=O)C(Br)C(=O)[C@@H](NC(=O)OCc1ccccc1)C(C1CC1)C1CC1]

To a solution of tert-butyl (S)-4-(((benzyloxy)carbonyl)amino)-5,5-dicyclopropyl-3-oxopentanoate (15.0 g, 37.3 mmol, 1.00 eq) in MeOH (150 mL) was added NBS (5.32 g, 29.8 mmol, 0.800 eq) and 2,6-dimethylpyridine (321 mg, 2.99 mmol, 348 mL, 0.0800 eq) at 0° C. The reaction mixture was stirred at 15° C. for 2 h. The reaction mixture was concentrated under reduced pressure to afford tert-butyl (4S)-4-(((benzyloxy)carbonyl)amino)-2-bromo-5,5-dicyclopropyl-3-oxopentanoate (15.0 g, 31.2 mmol, 84% yield) as a yellow solid. LCMS [M+H]⁺=480.2 m/z.

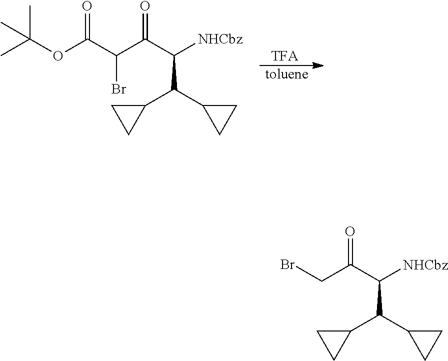 [SMILES: CC(C)(C)OC(=O)C(Br)C(=O)[C@@H](NC(=O)OCc1ccccc1)C(C1CC1)C1CC1 ; O=C(N[C@H](C(=O)CBr)C(C1CC1)C1CC1)OCc1ccccc1]

To a solution of tert-butyl (4S)-4-(((benzyloxy)carbonyl)amino)-2-bromo-5,5-dicyclopropyl-3-oxopentanoate (15.0 g, 31.2 mmol, 1.00 eq) in toluene (150 mL) was added TFA (17.8 g, 156 mmol, 11.5 mL, 5.00 eq). The reaction mixture was stirred at 80° C. for 2 h. The residue was diluted with a saturated aqueous solution of NaCO₃ to pH=7 and extracted with EtOAc, dried over anhydrous Na₂SO₄, filtered, and concentrated under reduced pressure to give a residue, which was purified by prep-HPLC (neutral condition; column: Kromasil Eternity XT (250 mm×80 mm, 10 mm); mobile phase: [water(NH₄HCO₃)-ACN]; B %: 45%-75%) to afford benzyl (S)-(4-bromo-1,1-dicyclopropyl-3-oxobutan-2-yl)carbamate. The title compound was isolated as the first eluting, single stereoisomer by chiral SFC purification (column: Daicel Chiralcel OJ (250 mm×50 mm, 10 mm); mobile phase: [Neu-MeOH]; B %: 20%-20%, 4.3 min); (6.00 g, 15.7 mmol, 50% yield) and was obtained as a white solid. ¹H NMR (400 MHz, CDCl₃) δ 7.43-7.31 (m, 5H), 5.65-5.55 (d, J=8.0 Hz, 1H), 5.12 (s, 2H), 4.88-4.78 (m, 1H), 4.26-4.10 (m, 2H), 0.79-0.63 (m, 3H), 0.59-0.41 (m, 4H), 0.30-0.14 (m, 4H). LCMS [M+H]⁺=380.1 m/z.

### Example 2: Preparation of Intermediate 2

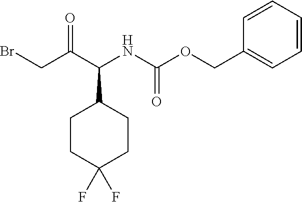 [SMILES: O=C(N[C@H](C(=O)CBr)C1CCC(F)(F)CC1)OCc1ccccc1]

### Benzyl(S)-(3-bromo-1-(4,4-difluorocyclohexyl)-2-oxopropyl)carbamate. (Intermediate 2, Int. 2)

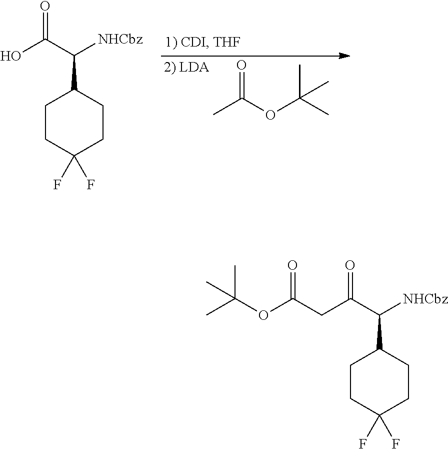

A solution of (S)-2-(((benzyloxy)carbonyl)amino)-2-(4,4-difluorocyclohexyl)acetic acid (30.0 g, 91.6 mmol, 1.00 eq) in THE (320 mL) and 1,1′-carbonyldiimidazole (16.3 g, 100 mmol, 1.10 eq) was stirred at 0° C. for 2 h. Meanwhile, to a solution of tert-butyl acetate (31.9 g, 274 mmol, 36.8 mL, 3.00 eq) in THE (200 mL) was added a solution of LDA (2 M, 160 mL, 3.50 eq), and the mixture was stirred at −70° C. for 1 h. The two reaction mixtures were mixed and stirred at −70° C. for 1 h. The reaction mixture was diluted with a saturated aqueous solution of NH₄Cl, extracted with EtOAc, washed with brine, dried over anhydrous Na₂SO₄, filtered, and concentrated under reduced pressure to give a residue which was purified by column chromatography (SiO₂, PE/EtOAc) to afford tert-butyl (S)-4-(((benzyloxy)carbonyl)amino)-4-(4,4-difluorocyclohexyl)-3-oxobutanoate (30.0 g, 70.5 mmol, 77% yield) as a yellow oil. LCMS [M+Na]⁺=448.1 m/z.

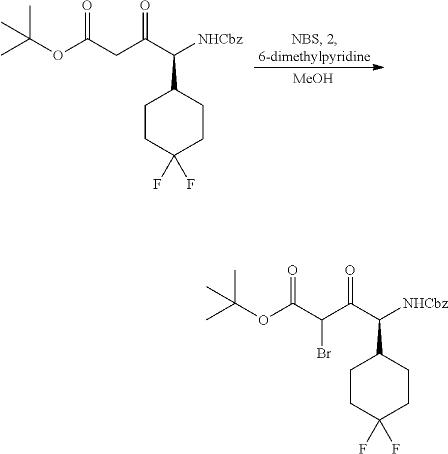 [SMILES: "MeOH" ; CC(C)(C)OC(=O)CC(=O)[C@@H](NC(=O)OCc1ccccc1)C1CCC(F)(F)CC1 ; CC(C)(C)OC(=O)C(Br)C(=O)[C@@H](NC(=O)OCc1ccccc1)C1CCC(F)(F)CC1]

To a solution of tert-butyl (S)-4-(((benzyloxy)carbonyl)amino)-4-(4,4-difluorocyclohexyl)-3-oxobutanoate (30.0 g, 70.5 mmol, 1.00 eq) in MeOH (300 mL) was added NBS (10.0 g, 56.4 mmol, 0.800 eq) and 2,6-dimethylpyridine (610 mg, 5.64 mmol, 0.08 eq) at 0° C. The mixture was stirred at RT for 2 h. The reaction was diluted with a saturated aqueous solution of NaCO₃, extracted with EtOAc, dried over anhydrous Na₂SO₄, filtered, and concentrated under reduced pressure to afford tert-butyl (4S)-4-(((benzyloxy)carbonyl)amino)-2-bromo-4-(4,4-difluorocyclohexyl)-3-oxobutanoate (20.0 g, 39.6 mmol, 56% yield) as a yellow solid, which was used in the next step without further purification

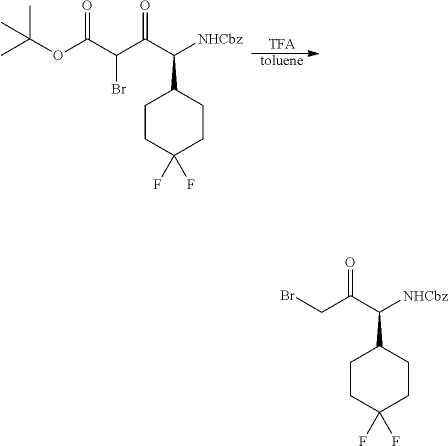 [SMILES: CC(C)(C)OC(=O)C(Br)C(=O)[C@@H](NC(=O)OCc1ccccc1)C1CCC(F)(F)CC1 ; O=C(N[C@H](C(=O)CBr)C1CCC(F)(F)CC1)OCc1ccccc1]

To a solution of tert-butyl (4S)-4-(((benzyloxy)carbonyl)amino)-2-bromo-4-(4,4-difluorocyclohexyl)-3-oxobutanoate (20.0 g, 39.6 mmol, 1.00 eq) in toluene (300 mL) was added TFA (27.1 g, 237 mmol, 17.6 mL, 6.00 eq). The reaction mixture was stirred at 75° C. for 2 h. The reaction mixture was diluted with a saturated aqueous solution of NaCO₃, and extracted with EtOAc, dried over anhydrous Na₂SO₄, filtered, and concentrated under reduced pressure to give a residue which was purified by prep-HPLC (basic condition; column: Kromasil Eternity XT (250 mm×80 mm, 10 mm); mobile phase: [water (NH₄HCO₃)-ACN]; B %: 45%-70%, 20 min) to afford benzyl (S)-(3-bromo-1-(4,4-difluorocyclohexyl)-2-oxopropyl)carbamate. The title compound was isolated as the first eluting, single stereoisomer by chiral SFC purification (column: Daicel Chiralcel OJ (250 mm×50 mm, 10 mm); mobile phase: [Neu-MeOH]; B %: 20%-20%, 4.3 min); (12.0 g, 26.4 mmol, 67% yield) and was obtained as a white solid. ¹H NMR (400 MHz, CDCl₃) δ 7.04-7.34 (m, 5H), 5.34 (d, J=8.8 Hz, 1H), 5.12 (s, 2H), 4.67 (dd, J=5.2 Hz, J=8.8 Hz, 1H), 4.03 (d, J=7.2 Hz, 2H), 2.15-2.12 (m, 2H), 1.97-1.94 (m, 1H), 1.81-1.35 (m, 6H). LCMS [M+H]⁺=404.0 m/z.

### Example 3: Preparation of Intermediate 3

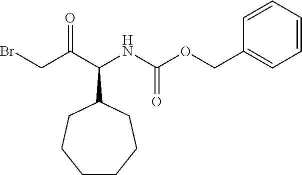 [SMILES: O=C(N[C@H](C(=O)CBr)C1CCCCCC1)OCc1ccccc1]

### Benzyl (S)-(3-bromo-1-cycloheptyl-2-oxopropyl)carbamate. (Intermediate 3, Int. 3)

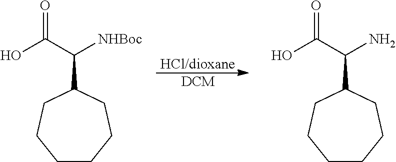 [SMILES: CC(C)(C)OC(=O)N[C@H](C(=O)O)C1CCCCCC1 ; N[C@H](C(=O)O)C1CCCCCC1]

To a solution of (S)-2-((tert-butoxycarbonyl)amino)-2-cycloheptylacetic acid (45.0 g 147 mmol, 1.00 eq) in DCM (400 mL) was added HCl in 1,4-dioxane (4.00 M, 368 mL, 10.0 eq) at 0° C. The mixture was stirred at RT for 3 h. The reaction mixture was concentrated under reduced pressure to afford (S)-2-amino-2-cycloheptylacetic acid (30.0 g, 144 mmol, 98% yield, HCl salt) as a white solid. LCMS [M+H]⁺=172.2 m/z.

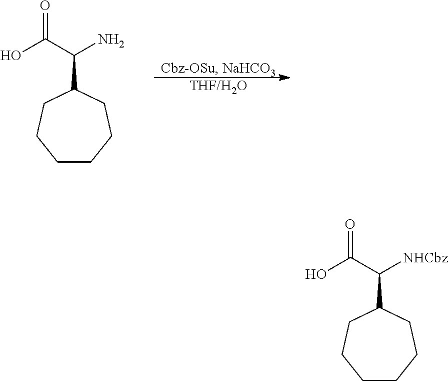 [SMILES: N[C@H](C(=O)O)C1CCCCCC1 ; O=C(N[C@H](C(=O)O)C1CCCCCC1)OCc1ccccc1]

To a solution of (S)-2-amino-2-cycloheptylacetic acid (30.0 g, 144 mmol, 1.00 eq, HCl salt) in THF (300 mL) and water (100 mL) was added benzyl (2,5-dioxopyrrolidin-1-yl) carbonate (71.9 g, 288 mmol, 2.00 eq) and NaCO₃ (36.4 g, 433 mmol, 16.8 mL, 3.00 eq). The reaction mixture was stirred at RT for 2 h. The reaction mixture was diluted with a 1 M aqueous solution of HCl to adjust pH=2 before extracting with DCM. The combined organic layers were dried over anhydrous Na₂SO₄, filtered, and concentrated under reduced pressure to give a residue, which was purified by column chromatography (SiO₂, PE/EtOAc) to afford (S)-2-(((benzyloxy)carbonyl)amino)-2-cycloheptylacetic acid (43.0 g, 140 mmol, 97% yield) as a white solid. ¹H NMR (400 MHz, CDCl₃) δ 7.42-7.31 (m, 5H), 5.27 (d, J=9.2 Hz, 1H), 5.13 (s, 2H), 4.40-4.37 (dd, J1=4.0, J2=8.8 Hz, 1H), 2.19-2.05 (d, J=3.5 Hz, 1H), 1.80-1.30 (m, 12H). LCMS [M+H]⁺=306.1 m/z.

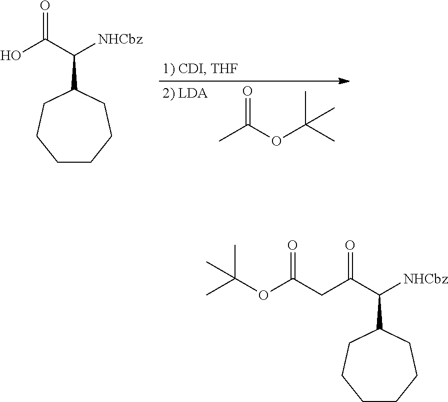

A solution of (S)-2-(((benzyloxy)carbonyl)amino)-2-cycloheptylacetic acid (56.0 g, 183 mmol, 1.00 eq) and 1,1′-carbonyldiimidazole (32.7 g, 201 mmol, 1.10 eq) in THF (600 mL) was stirred at 0° C. for 2 h. Meanwhile, to a solution of tert-butyl acetate (63.9 g, 550 mmol, 73.7 mL, 3.00 eq) in THF (500 mL) was added a solution of LDA (2 M, 275 mL, 3.00 eq), and the reaction mixture was stirred at −78° C. for 2 h. The two reaction mixtures were combined and stirred at −78° C. for 2 h. The reaction mixture was treated with a saturated aqueous solution of NH₄Cl and extracted with EtOAc, dried over anhydrous Na₂SO₄, filtered, and concentrated under reduced pressure to give a residue, which was purified by column chromatography (SiO₂, PE/EtOAc) to afford tert-butyl (S)-4-(((benzyloxy)carbonyl)amino)-4-cycloheptyl-3-oxobutanoate (36.0 g, 89.2 mmol, 49% yield) as a white solid. ¹H NMR (400 MHz, CDCl₃) δ 7.39-7.31 (m, 5H), 5.37 (d, J=8.8 Hz, 1H), 5.12 (s, 2H), 4.50-4.47 (dd, J1=3.6, J2=8.8 Hz, 1H), 3.48-3.42 (m, 2H), 2.14-2.06 (m, 1H), 1.85-1.55 (m, 7H), 1.49-1.44 (m, 11H), 1.39-1.13 (m, 3H). LCMS [M+Na]⁺=426.2 m/z.

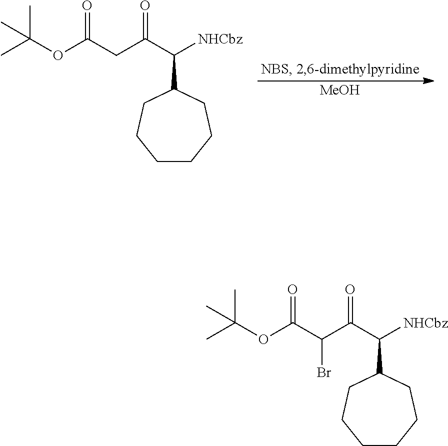 [SMILES: "MeOH" ; CC(C)(C)OC(=O)CC(=O)[C@@H](NC(=O)OCc1ccccc1)C1CCCCCC1 ; CC(C)(C)OC(=O)C(Br)C(=O)[C@@H](NC(=O)OCc1ccccc1)C1CCCCCC1]

To a solution of tert-butyl (S)-4-(((benzyloxy)carbonyl)amino)-4-cycloheptyl-3-oxobutanoate (36.0 g, 89.2 mmol, 1.00 eq) in MeOH (400 mL) was added NBS (13.5 g, 75.8 mmol, 0.85 eq) and 2,6-dimethylpyridine (771 mg, 7.14 mmol, 0.08 eq) at 0° C. The reaction mixture was stirred at 15° C. for 2 h. The reaction mixture was diluted with water and extracted with EtOAc. The combined organic layers were washed with a saturated aqueous solution of NaCO₃, dried over anhydrous Na₂SO₄, filtered, and concentrated under reduced pressure to afford tert-butyl (4S)-4-(((benzyloxy)carbonyl)amino)-2-bromo-4-cycloheptyl-3-oxobutanoate (40.0 g, 82.9 mmol, 93% yield) as a yellow oil, which was used directly in the next step without further purification.

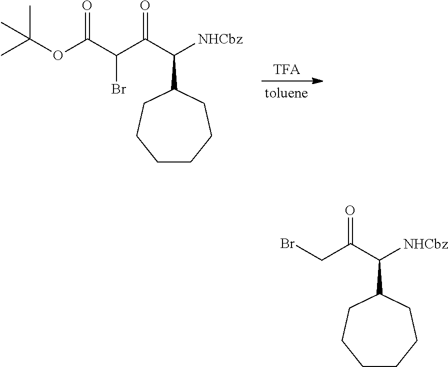 [SMILES: CC(C)(C)OC(=O)C(Br)C(=O)[C@@H](NC(=O)OCc1ccccc1)C1CCCCCC1 ; O=C(N[C@H](C(=O)CBr)C1CCCCCC1)OCc1ccccc1]

To a solution of tert-butyl (4S)-4-(((benzyloxy)carbonyl)amino)-2-bromo-4-cycloheptyl-3-oxobutanoate (40.0 g, 82.9 mmol, 1.00 eq) in toluene (400 mL) was added TFA (56.7 g, 497 mmol, 36.8 mL, 6.00 eq) at 0° C. The reaction mixture was stirred at 75° C. for 2 h. The reaction mixture was diluted with water, and a saturated aqueous solution of NaCO₃ was added to adjust the pH=9. The reaction mixture was extracted with DCM, and the combined organic layers were dried over anhydrous Na₂SO₄, filtered, and concentrated under reduced pressure to give a residue, which was purified by prep-HPLC (basic condition; column: Kromasil Eternity XT (250 mm×80 mm, 10 mm); mobile phase: [water (NH₄OH)-ACN]; B %: 55%-85%, 20 min) to afford benzyl (S)-(3-bromo-1-cycloheptyl-2-oxopropyl)carbamate. The title compound was isolated as the first eluting, single stereoisomer by chiral SFC purification (column: Daicel Chiralcel OJ (250 mm×30 mm, 10 mm); mobile phase: [0.1% NH₃H₂O in IPA]; B %: 20%-20%, 3 min); (11.3 g, 29.5 mmol, 36% yield) and was obtained as a white solid. ¹H NMR (400 MHz, MeOD) δ 7.39-7.27 (m, 5H), 5.10 (s, 2H), 4.45-4.37 (m, 1H), 4.22 (s, 2H), 2.21-2.04 (m, 1H), 1.70-1.28 (m, 12H). LCMS [M+H]⁺=384.2 m/z.

### Example 4: Preparation of Compound 1

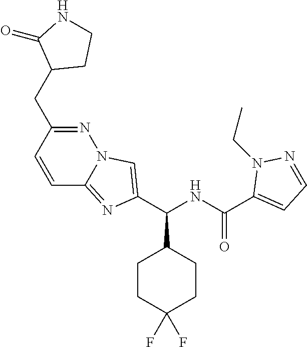 [SMILES: CCn1nccc1C(=O)N[C@H](c1cn2nc(CC3CCNC3=O)ccc2n1)C1CCC(F)(F)CC1]

### N-((1S)-(4,4-difluorocyclohexyl)(6-((2-oxopyrrolidin-3-yl)methyl)imidazo[1,2-b]pyridazin-2-yl)methyl)-1-ethyl-1H-pyrazole-5-carboxamide. (Compound 1)

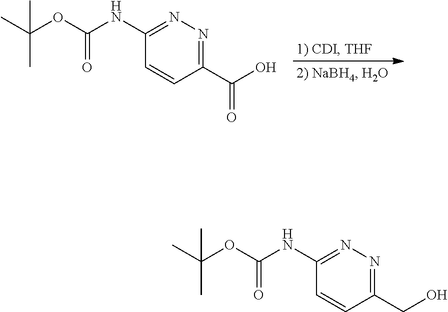 [SMILES: CC(C)(C)OC(=O)Nc1ccc(C(=O)O)nn1 ; CC(C)(C)OC(=O)Nc1ccc(CO)nn1]

To a solution of 6-((tert-butoxycarbonyl)amino)pyridazine-3-carboxylic acid (2.00 g, 8.36 mmol, 1.00 eq) in THF (80.0 mL) was added 1,1′-carbonyldiimidazole (4.28 g, 25.1 mmol, 3.00 eq). The resulting mixture was stirred at RT for 2 h and then cooled down to 0° C. for the addition of NaBH₄ (791 mg, 20.9 mmol, 2.50 eq) in water (10 mL). The reaction mixture was further stirred at RT for 2 h. After removal of the solvents, the residue was treated with water and extracted with EtOAc. The combined organics were washed with brine, dried over anhydrous Na₂SO₄, filtered and concentrated under reduced pressure to give a residue which was purified by column chromatography (SiO₂, PE/EtOAc) to afford tert-butyl (6-(hydroxymethyl)pyridazin-3-yl)carbamate (1.15 g, 5.1 mmol, 61% yield) as a colorless oil.

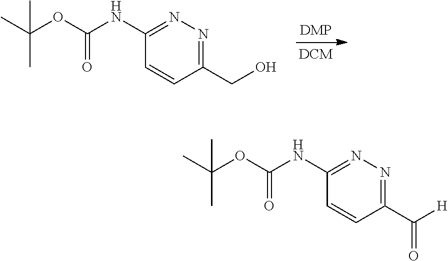 [SMILES: CC(C)(C)OC(=O)Nc1ccc(CO)nn1 ; CC(C)(C)OC(=O)Nc1ccc(C=O)nn1]

To a solution of tert-butyl (6-(hydroxymethyl)pyridazin-3-yl)carbamate (0.6 g, 2.66 mmol, 1.00 eq) in DCM (30 mL) was added Dess-Martin periodinane (1.22 g, 2.80 mmol, 1.05 eq). The reaction mixture was stirred at RT for 1 h. The reaction mixture was washed with 1 M aqueous solution of NaOH and the aqueous layer was extracted with EtOAc. The combined organic layers were washed with brine, dried over anhydrous Na₂SO₄, filtered, and concentrated under reduced pressure to afford tert-butyl (6-formylpyridazin-3-yl)carbamate (0.4 g, 1.78 mmol, 67% yield) as a brown solid. ¹H NMR (400 MHz, CDCl₃) δ 10.24 (s, 1H), 8.39 (d, J=9.28 Hz, 1H), 8.01 (d, J=9.38 Hz, 1H), 1.56 (s, 9H).

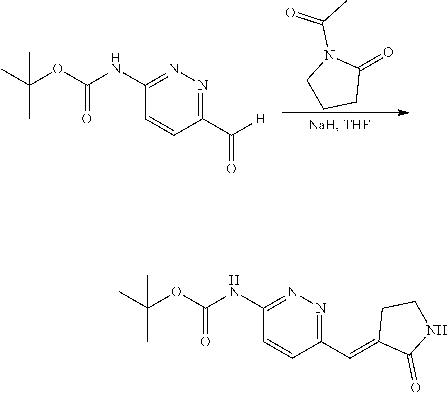

To a solution of tert-butyl (6-formylpyridazin-3-yl)carbamate (1.25 g, 9.86 mmol, 1.00 eq) in THF (44.0 mL) was added NaH (788 mg, 19.7 mmol, 2.00 eq) at 0° C. followed by the addition of 1-acetylpyrrolidin-2-one (1.10 g, 4.93 mmol, 0.5 eq). The reaction mixture was warmed up to RT and stirred for 16 h. The reaction mixture was quenched by iced water and extracted with EtOAc. The combined organic layers were washed with brine, dried over anhydrous Na₂SO₄, filtered, and concentrated under reduced pressure to give a residue which was purified by column chromatography (SiO₂, DCM/MeOH) to afford tert-butyl (E)-(6-((2-oxopyrrolidin-3-ylidene)methyl)pyridazin-3-yl)carbamate (540 mg, 3.75 mmol, 38% yield) as a colorless oil. LCMS [M+H]⁺=291.1 m/z.

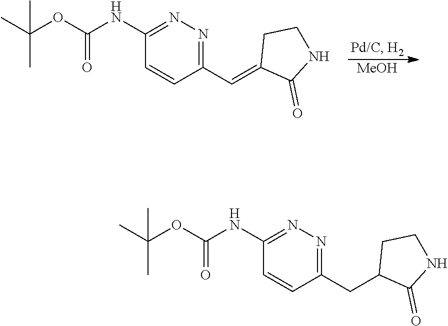 [SMILES: "MeOH" ; CC(C)(C)OC(=O)Nc1ccc(/C=C2\CCNC2=O)nn1 ; CC(C)(C)OC(=O)Nc1ccc(CC2CCNC2=O)nn1]

To a solution of tert-butyl (E)-(6-((2-oxopyrrolidin-3-ylidene)methyl)pyridazin-3-yl)carbamate (20 mg, 0.069 mmol, 1.00 eq) in MeOH (10 mL) was added Pd/C (36.7 mg, 10% purity) and the reaction mixture was first purged with argon followed by evacuation and purging with H₂ (3 times). The reaction mixture was stirred under H₂ pressure for 16 h. The reaction mixture was filtered through a Celite pad and concentrated under reduced pressure to obtain a residue which was purified by column chromatography (SiO₂, DCM/MeOH) to afford tert-butyl (6-((2-oxopyrrolidin-3-yl)methyl)pyridazin-3-yl)carbamate (14 mg, 0.048 mmol, 70% yield) as a yellow solid. ¹H NMR (400 MHz, CDCl₃) δ 8.12 (d, J=9.18 Hz, 1H), 7.69 (s, 1H), 7.38 (d, J=9.20 Hz, 1H), 5.90 (s, 1H), 3.40 (dd, J=14.27, 4.22 Hz, 1H), 3.20-3.31 (m, 2H), 3.01 (dd, J=14.27, 8.91 Hz, 1H), 2.90 (qd, J=8.93, 4.20 Hz, 1H), 2.23-2.31 (m, 1H), 1.94 (dq, J=12.92, 8.84 Hz, 1H), 1.51 (s, 9H). LCMS [M+H]⁺=293.2 m/z.

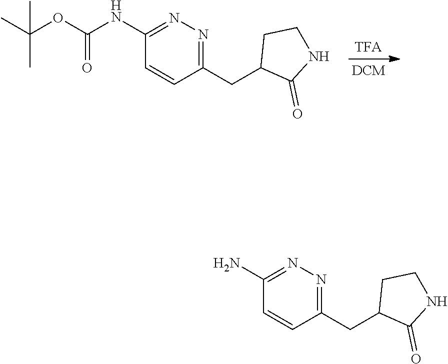 [SMILES: CC(C)(C)OC(=O)Nc1ccc(CC2CCNC2=O)nn1 ; Nc1ccc(CC2CCNC2=O)nn1]

To a solution of tert-butyl (6-((2-oxopyrrolidin-3-yl)methyl)pyridazin-3-yl)carbamate (60 mg, 0.205 mmol, 1.00 eq) in DCM (1.5 mL) was added TFA (1.5 mL). The reaction mixture was stirred at RT for 2 h. The reaction mixture was then concentrated under reduced pressure to afford 3-((6-aminopyridazin-3-yl)methyl)pyrrolidin-2-one (60 mg, 0.196 mmol, 95% yield, TFA salt) as light pink solid. LCMS [M+H]⁺=193.1 m/z.

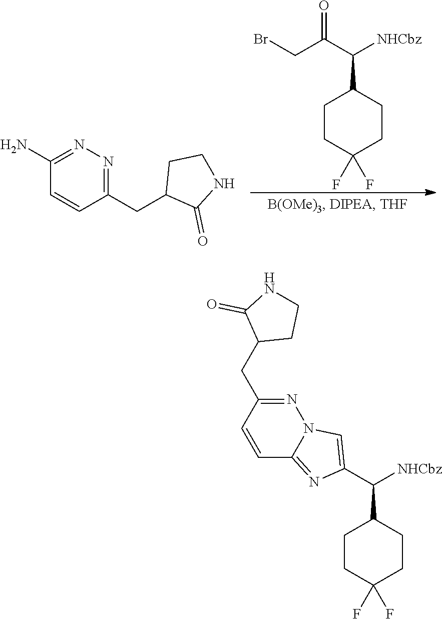 [SMILES: Nc1ccc(CC2CCNC2=O)nn1 ; O=C(N[C@H](C(=O)CBr)C1CCC(F)(F)CC1)OCc1ccccc1 ; O=C(N[C@H](c1cn2nc(CC3CCNC3=O)ccc2n1)C1CCC(F)(F)CC1)OCc1ccccc1]

To a solution of 3-((6-aminopyridazin-3-yl)methyl)pyrrolidin-2-one (60 mg, 0.196 mmol, 1.47 eq, TFA salt) and benzyl (S)-(3-bromo-1-(4,4-difluorocyclohexyl)-2-oxopropyl)carbamate (Int. 2; 50 mg, 0.124 mmol, 1.00 eq) in THF (2.5 mL) was added trimethyl borate (57 mg, 0.534 mmol, 0.06 mL, 4.00 eq) and DIEA (86 mg, 0.667 mmol, 0.116 mL, 5.00 eq). The reaction mixture was stirred at 75° C. for 1 h. The reaction mixture was concentrated under reduced pressure to give a residue which was purified by column chromatography (SiO₂, DCM/MeOH) to afford benzyl ((1S)-(4,4-difluorocyclohexyl)(6-((2-oxopyrrolidin-3-yl)methyl)imidazo[1,2-b]pyridazin-2-yl)methyl)carbamate (40 mg, 0.08 mmol, 65% yield) as a yellow oil. LCMS [M+H]⁺=498.2 m/z.

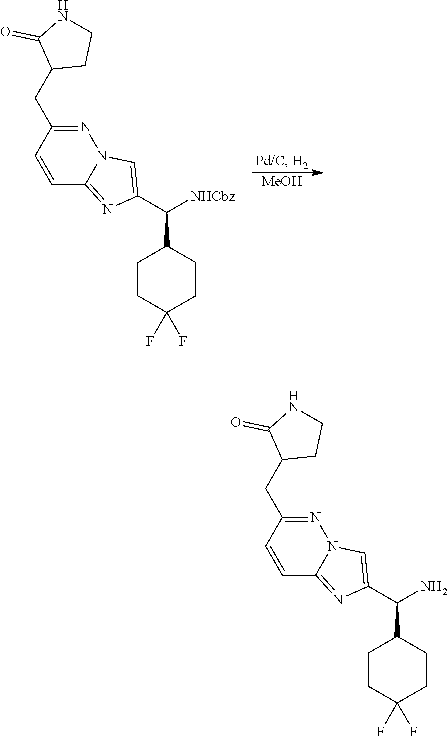 [SMILES: "MeOH" ; O=C(N[C@H](c1cn2nc(CC3CCNC3=O)ccc2n1)C1CCC(F)(F)CC1)OCc1ccccc1 ; N[C@H](c1cn2nc(CC3CCNC3=O)ccc2n1)C1CCC(F)(F)CC1]

To a solution of benzyl ((1S)-(4,4-difluorocyclohexyl)(6-((2-oxopyrrolidin-3-yl)methyl)imidazo[1,2-b]pyridazin-2-yl)methyl)carbamate (40 mg, 0.08 mmol, 1.00 eq) in MeOH (20 mL) was added Pd/C (43 mg, 10% purity) and the reaction mixture was first purged with argon followed by evacuation and purging with H₂ (3 times). The reaction mixture was stirred under H₂ pressure for 30 h. The reaction mixture was filtered through a Celite pad and concentrated under reduced pressure to afford 3-((2-((S)-amino(4,4-difluorocyclohexyl)methyl)imidazo[1,2-b]pyridazin-6-yl)methyl)pyrrolidin-2-one (29 mg, 0.08 mmol, 99% yield) as a yellow solid. LCMS [M+H]⁺=364.2 m/z.

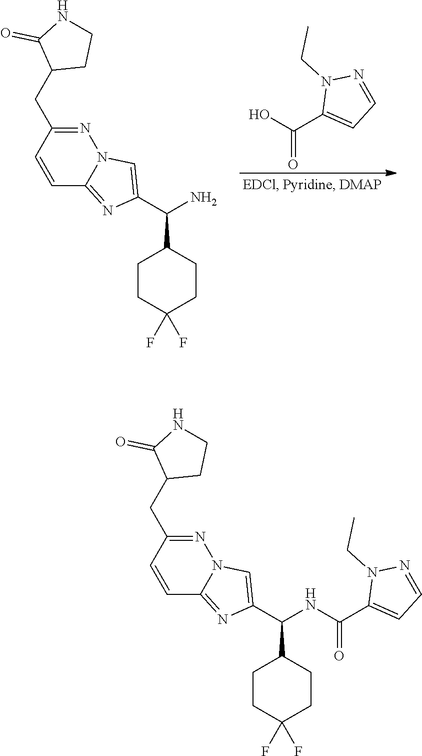

To a solution of 3-((2-((S)-amino(4,4-difluorocyclohexyl)methyl)imidazo[1,2-b]pyridazin-6-yl)methyl)pyrrolidin-2-one (29 mg, 0.08 mmol, 1.00 eq) and 1-ethyl-1H-pyrazole-5-carboxylic acid (22.4 mg, 0.160 mmol, 2.00 eq) in pyridine (2.5 mL) was added EDCI (61.2 mg, 0.319 mmol, 4.00 eq) and 4-dimethylaminopyridine (4.9 mg, 0.040 mmol, 0.50 eq). The reaction mixture was stirred at RT for 1 h. The reaction mixture was concentrated under reduced pressure, and the residue was dissolved in water and extracted with EtOAc. The combined organic layers were washed with brine, dried over Na₂SO₄, filtered, and concentrated under reduced pressure to give a residue, which was purified by column chromatography (SiO₂, DCM/MeOH) to afford N-((1S)-(4,4-difluorocyclohexyl)(6-((2-oxopyrrolidin-3-yl)methyl)imidazo[1,2-b]pyridazin-2-yl)methyl)-1-ethyl-1H-pyrazole-5-carboxamide. The title compound was isolated as the first eluting, single stereoisomer by chiral SFC purification (column: Daicel Chiralpak AS (250 mm×30 mm, 10 mm); mobile phase: [0.1% NH₃H₂O in MeOH]; B %: 40%-40%, 3 min); (2.71 mg, 5.58 mmol, 37% yield, 89% purity) and was obtained as a white solid. LCMS [M+H]+=486.3 m/z.

### Example 5: Preparation of Compound 2

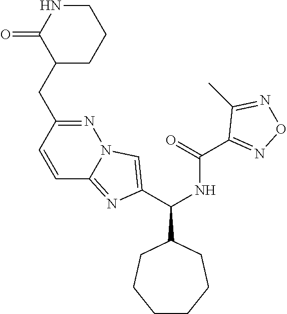 [SMILES: Cc1nonc1C(=O)N[C@H](c1cn2nc(CC3CCCNC3=O)ccc2n1)C1CCCCCC1]

### N-((1S)-cycloheptyl(6-((2-oxopiperidin-3-yl)methyl)imidazo[1,2-b]pyridazin-2-yl)methyl)-4-methyl-1,2,5-oxadiazole-3-carboxamide. (Compound 2)

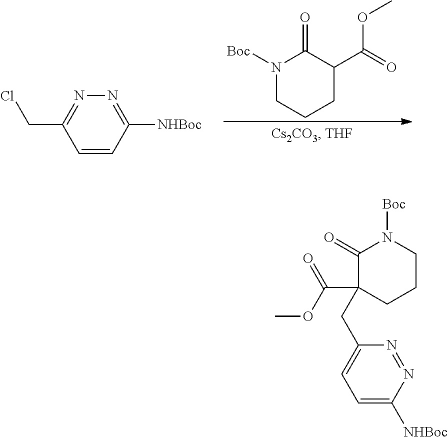 [SMILES: CC(C)(C)OC(=O)Nc1ccc(CCl)nn1 ; COC(=O)C1CCCN(C(=O)OC(C)(C)C)C1=O ; COC(=O)C1(Cc2ccc(NC(=O)OC(C)(C)C)nn2)CCCN(C(=O)OC(C)(C)C)C1=O]

To a solution of tert-butyl (6-(chloromethyl)pyridazin-3-yl)carbamate (1.27 g, 4.92 mmol, 1.20 eq) in THF (20.0 mL) was added Cs₂CO₃ (2.67 g, 8.21 mmol, 2.00 eq). The resulting reaction mixture was stirred at 20° C. for 1 h before adding 1-(tert-butyl) 3-methyl 2-oxopiperidine-1,3-dicarboxylate (1.00 g, 4.10 mmol, 1.00 eq). The reaction mixture was stirred at 70° C. for 2 h. The reaction mixture was diluted with water and extracted with EtOAc, dried over anhydrous Na₂SO₄, filtered, and concentrated under reduced pressure to give a residue, which was purified by re-crystallization from EtOAc at 20° C. to afford 1-(tert-butyl) 3-methyl 3-((6-((tert-butoxycarbonyl)amino)pyridazin-3-yl)methyl)-2-oxopiperidine-1,3-dicarboxylate (1.10 g, 2.37 mmol, 58% yield) as a light-yellow solid. ¹H NMR (400 MHz, CDCl₃) δ 8.09 (d, J=9.2 Hz, 1H), 7.65 (s, 1H), 7.45 (d, J=9.2 Hz, 1H), 3.75 (s, 3H), 3.64-3.59 (m, 2H), 3.50-3.45 (m, 2H), 2.25-2.08 (m, 2H), 1.90-1.76 (m, 2H), 1.52 (s, 9H), 1.49 (s, 9H). LCMS [M+H]⁺=465.4 m/z.

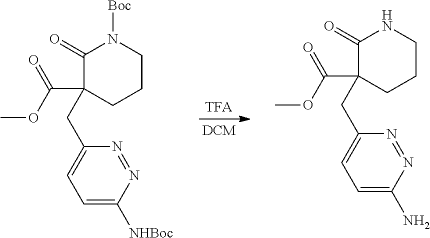 [SMILES: COC(=O)C1(Cc2ccc(NC(=O)OC(C)(C)C)nn2)CCCN(C(=O)OC(C)(C)C)C1=O ; COC(=O)C1(Cc2ccc(N)nn2)CCCNC1=O]

To a solution of 1-(tert-butyl) 3-methyl 3-((6-((tert-butoxycarbonyl)amino)pyridazin-3-yl)methyl)-2-oxopiperidine-1,3-dicarboxylate (1.00 g, 2.15 mmol, 1.00 eq) in DCM (5.00 mL) was added TFA (15.4 g, 135 mmol, 10.0 mL, 62.7 eq) at 0° C., the reaction mixture was stirred at 20° C. for 2 h. The reaction mixture was filtered and concentrated under reduced pressure to afford methyl 3-((6-aminopyridazin-3-yl)methyl)-2-oxopiperidine-3-carboxylate (1.10 g, crude, TFA salt) as a yellow oil. LCMS [M+H]⁺=265.1 m/z.

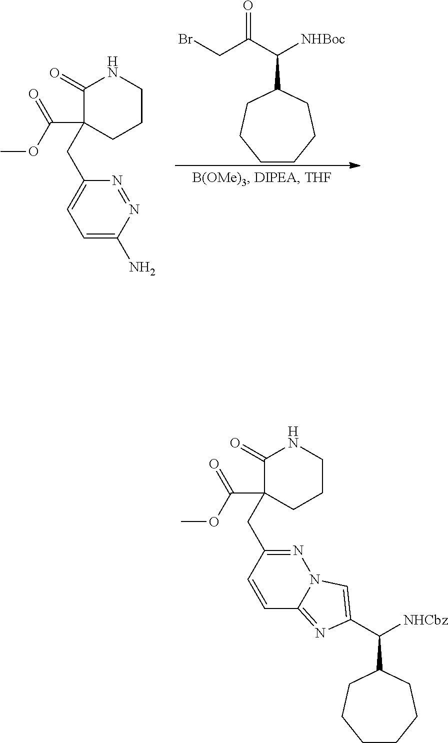 [SMILES: COC(=O)C1(Cc2ccc(N)nn2)CCCNC1=O ; CC(C)(C)OC(=O)N[C@H](C(=O)CBr)C1CCCCCC1 ; COC(=O)C1(Cc2ccc3nc([C@@H](NC(=O)OCc4ccccc4)C4CCCCCC4)cn3n2)CCCNC1=O]

To a solution of methyl 3-((6-aminopyridazin-3-yl)methyl)-2-oxopiperidine-3-carboxylate (700 mg, 1.85 mmol, 1.00 eq, TFA salt) and benzyl (S)-(3-bromo-1-cycloheptyl-2-oxopropyl)carbamate (Int. 3; 707 mg, 1.85 mmol, 1.00 eq) in THF (10.0 mL) was added trimethyl borate (961 mg, 9.25 mmol, 1.04 mL, 5.00 eq) and DIEA (2.39 g, 18.5 mmol, 3.22 mL, 10.0 eq). The reaction mixture was stirred at 70° C. for 3 h. The reaction mixture was diluted with water and extracted with EtOAc, dried over anhydrous Na₂SO₄, filtered, and concentrated under reduced pressure to give a residue, which was purified by prep-HPLC (column: Phenomenex luna C18 150*40 mm*15 mm; mobile phase: [water (FA)-ACN]; B %: 42%-72%, 10 min) to afford methyl 3-((2-((S)-(((benzyloxy)carbonyl)amino)(cycloheptyl)methyl)imidazo[1,2-b]pyridazin-6-yl)methyl)-2-oxopiperidine-3-carboxylate (350 mg, 639 mmol, 35% yield) as a yellow solid. LCMS [M+H]⁺=548.5 m/z.

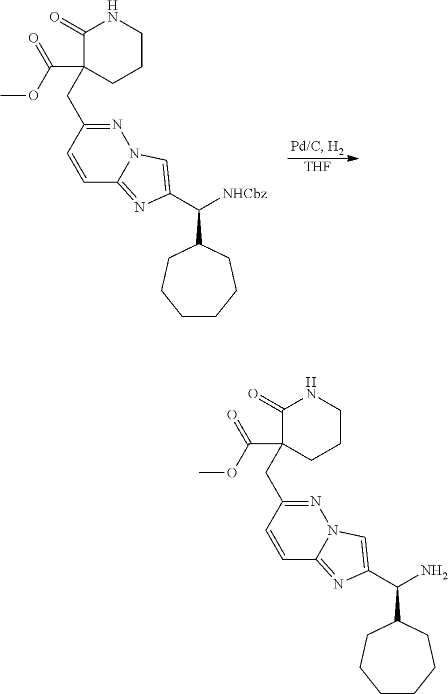 [SMILES: COC(=O)C1(Cc2ccc3nc([C@@H](NC(=O)OCc4ccccc4)C4CCCCCC4)cn3n2)CCCNC1=O ; COC(=O)C1(Cc2ccc3nc([C@@H](N)C4CCCCCC4)cn3n2)CCCNC1=O ; "THF"]

To a solution of methyl 3-((2-((S)-(((benzyloxy)carbonyl)amino (cycloheptyl)methyl)imidazo[1,2-b]pyridazin-6-yl)methyl)-2-oxopiperidine-3-carboxylate (350 mg, 639 mmol, 1.00 eq) in THF (5.00 mL) was added Pd/C (100 mg, 10.0% purity). The reaction mixture was stirred at 20° C. for 3 h under H₂ (25 psi). The reaction mixture was filtered through a pad of Celite and concentrated under reduced pressure to afford methyl 3-((2-((S)-amino (cycloheptyl)methyl)imidazo[1,2-b]pyridazin-6-yl)methyl)-2-oxopiperidine-3-carboxylate (200 mg, 484 mmol, 76% yield) as a light-yellow solid. LCMS [M+H]⁺=414.2 m/z.

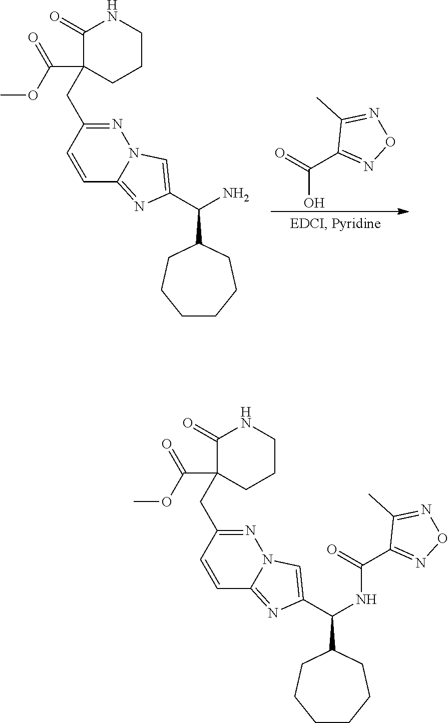

To a solution of methyl 3-((2-((S)-amino(cycloheptyl)methyl)imidazo[1,2-b]pyridazin-6-yl)methyl)-2-oxopiperidine-3-carboxylate (100 mg, 242 mmol, 1.00 eq) and 4-methyl-1,2,5-oxadiazole-3-carboxylic acid (46.5 mg, 363 mmol, 1.50 eq) in pyridine (3.00 mL) was added EDCI (139 mg, 725 mmol, 3.00 eq). The reaction mixture was stirred at 20° C. for 2 h. The reaction mixture was diluted with water and extracted with EtOAc, dried over anhydrous Na₂SO₄, filtered, and concentrated under reduced pressure to give a residue, which was purified by prep-TLC (SiO₂, DCM/MeOH) to afford methyl 3-((2-((S)-cycloheptyl(4-methyl-1,2,5-oxadiazole-3-carboxamido)methyl)imidazo[1,2-b]pyridazin-6-yl)methyl)-2-oxopiperidine-3-carboxylate (70.0 mg, 134 mmol, 55% yield) as a white solid. LCMS [M+H]⁺=524.2 m/z.

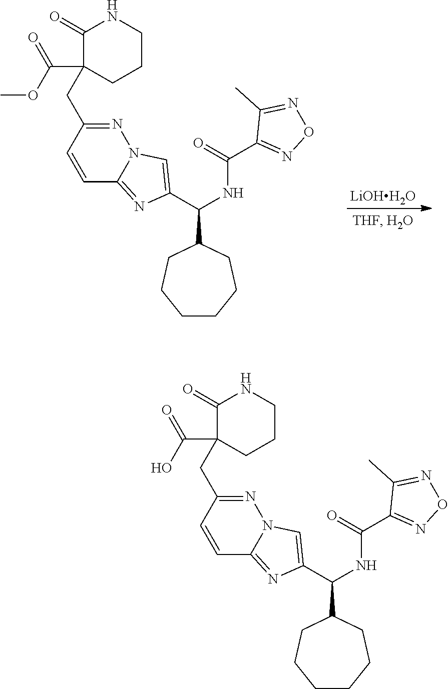 [SMILES: COC(=O)C1(Cc2ccc3nc([C@@H](NC(=O)c4nonc4C)C4CCCCCC4)cn3n2)CCCNC1=O ; "LiOH·H2O" ; Cc1nonc1C(=O)N[C@H](c1cn2nc(CC3(C(=O)O)CCCNC3=O)ccc2n1)C1CCCCCC1]

To a solution of methyl 3-((2-((S)-cycloheptyl(4-methyl-1,2,5-oxadiazole-3-carboxamido)methyl)imidazo[1,2-b]pyridazin-6-yl)methyl)-2-oxopiperidine-3-carboxylate (70.0 mg, 134 mmol, 1.00 eq) in THF (3.00 mL) and water (3.00 mL) was added LiOH·H₂O (16.8 mg 401 mmol, 3.00 eq). The reaction mixture was stirred at 20° C. for 3 h. The pH of the reaction mixture was adjusted to 5 with a 1 M HCl, extracted with EtOAc, dried over anhydrous Na₂SO₄, filtered, and concentrated under reduced pressure to afford 3-((2-((S)-cycloheptyl(4-methyl-1,2,5-oxadiazole-3-carboxamido)methyl)imidazo[1,2-b]pyridazin-6-yl)methyl)-2-oxopiperidine-3-carboxylic acid (55.0 mg, 108 mmol, 81% yield) as a white solid.

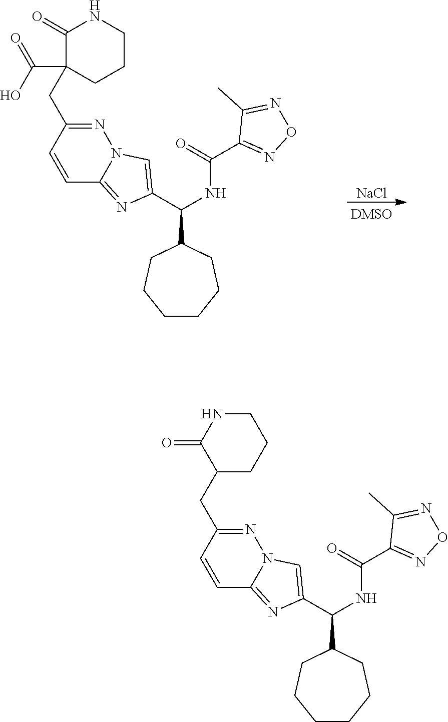 [SMILES: Cc1nonc1C(=O)N[C@H](c1cn2nc(CC3(C(=O)O)CCCNC3=O)ccc2n1)C1CCCCCC1 ; "NaCl" ; Cc1nonc1C(=O)N[C@H](c1cn2nc(CC3CCCNC3=O)ccc2n1)C1CCCCCC1]

To a solution of 3-((2-((S)-cycloheptyl(4-methyl-1,2,5-oxadiazole-3-carboxamido)methyl)imidazo[1,2-b]pyridazin-6-yl)methyl)-2-oxopiperidine-3-carboxylic acid (55.0 mg, 108 mmol, 1.00 eq) in DMSO (2.00 mL) was added NaCl (12.6 mg, 216 mmol, 2.00 eq). The reaction mixture was stirred at 120° C. for 2 h. The reaction mixture was then diluted with EtOAc and washed with water, and the combined organic layers were dried over Na₂SO₄, filtered, and concentrated under reduced pressure. The residue was purified by prep-TLC (SiO₂, DCM/MeOH) to afford N-((1S)-cycloheptyl(6-((2-oxopiperidin-3-yl)methyl)imidazo[1,2-b]pyridazin-2-yl)methyl)-4-methyl-1,2,5-oxadiazole-3-carboxamide (3.18 mg, 5.70 mmol, 5% yield, 84% purity) as a white solid. LCMS [M+H]⁺=466.2 m/z.

### Example 6: Preparation of Compound 3

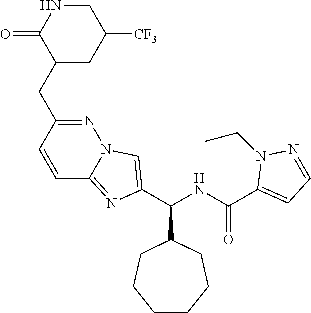 [SMILES: CCn1nccc1C(=O)N[C@H](c1cn2nc(CC3CC(C(F)(F)F)CNC3=O)ccc2n1)C1CCCCCC1]

### N-((1 S)-cycloheptyl(6-((2-oxo-5-(trifluoromethyl)piperidin-3-yl)methyl)imidazo[1,2-b]pyridazin-2-yl)methyl)-1-ethyl-1H-pyrazole-5-carboxamide. (Compound 3)

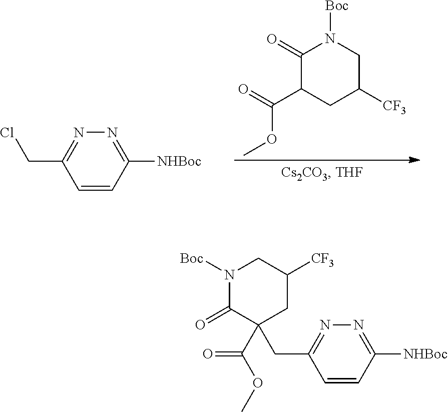 [SMILES: CC(C)(C)OC(=O)Nc1ccc(CCl)nn1 ; COC(=O)C1CC(C(F)(F)F)CN(C(=O)OC(C)(C)C)C1=O ; COC(=O)C1(Cc2ccc(NC(=O)OC(C)(C)C)nn2)CC(C(F)(F)F)CN(C(=O)OC(C)(C)C)C1=O]

To a solution of 1-(tert-butyl) 3-methyl 2-oxo-5-(trifluoromethyl)piperidine-1,3-dicarboxylate (350 mg, 1.08 mmol, 1.00 eq) in THE (4.00 mL) was added Cs₂CO₃ (1.40 g, 4.30 mmol, 4.00 eq). The reaction mixture was stirred at 15° C. for 30 min, then tert-butyl (6-(chloromethyl)pyridazin-3-yl)carbamate (262 mg, 1.08 mmol, 1.00 eq) was added. The reaction mixture was stirred at 55° C. for 12 h. The reaction mixture was diluted with water and extracted with EtOAc, dried over anhydrous Na₂SO₄, filtered, and concentrated under reduced pressure. The resulting residue was purified by prep-HPLC (column: Waters Xbridge C18 150*50 mm*10 mm; mobile phase: [water (NH4HCO3)-ACN]; B %: 45%-75%, 10 min) to afford 1-(tert-butyl) 3-methyl 3-((6-((tert-butoxycarbonyl)amino)pyridazin-3-yl)methyl)-2-oxo-5-(trifluoromethyl)piperidine-1,3-dicarboxylate (80.0 mg, 150 mmol, 14% yield) as a yellow oil solid. LCMS [M+H]⁺=533.3 m/z.

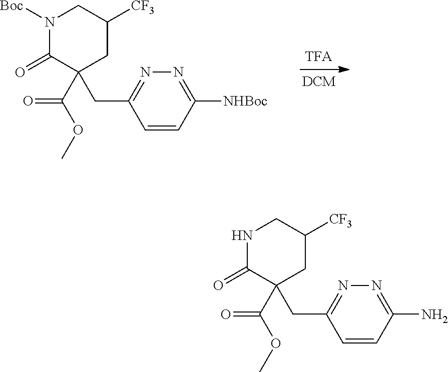 [SMILES: COC(=O)C1(Cc2ccc(NC(=O)OC(C)(C)C)nn2)CC(C(F)(F)F)CN(C(=O)OC(C)(C)C)C1=O ; COC(=O)C1(Cc2ccc(N)nn2)CC(C(F)(F)F)CNC1=O]

To a solution of 1-(tert-butyl) 3-methyl 3-((6-((tert-butoxycarbonyl)amino)pyridazin-3-yl)methyl)-2-oxo-5-(trifluoromethyl)piperidine-1,3-dicarboxylate (40.0 mg, 75.1 mmol, 1.00 eq) in DCM (0.50 mL) was added TFA (1.54 g, 13.5 mmol, 1.00 mL, 179 eq) at 0° C. The reaction mixture was stirred at 20° C. for 2 h. The reaction mixture was concentrated under reduced pressure to afford methyl 3-((6-aminopyridazin-3-yl)methyl)-2-oxo-5-(trifluoromethyl)piperidine-3-carboxylate (30 mg, crude, TFA salt) as a yellow solid.

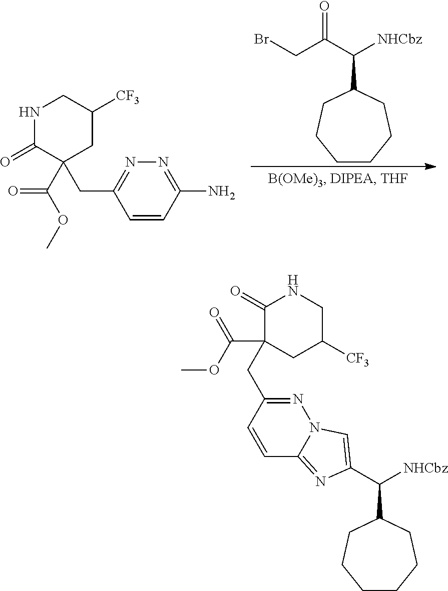 [SMILES: COC(=O)C1(Cc2ccc(N)nn2)CC(C(F)(F)F)CNC1=O ; O=C(N[C@H](C(=O)CBr)C1CCCCCC1)OCc1ccccc1 ; COC(=O)C1(Cc2ccc3nc([C@@H](NC(=O)OCc4ccccc4)C4CCCCCC4)cn3n2)CC(C(F)(F)F)CNC1=O]

To a solution of methyl 3-((6-aminopyridazin-3-yl)methyl)-2-oxo-5-(trifluoromethyl)piperidine-3-carboxylate (40.0 mg, 89.6 mmol, 1.00 eq, TFA salt) and benzyl (S)-(3-bromo-1-cycloheptyl-2-oxopropyl)carbamate (Int. 3; 55.2 mg, 144 mmol, 1.61 eq) in THF (2.00 mL) was added trimethyl borate (46.5 mg, 448 mmol, 50.6 mL, 5.00 eq) and DIEA (115 mg 896 mmol, 156 mL, 10.0 eq). The reaction mixture was stirred at 70° C. for 2 h. The reaction mixture was diluted with water and extracted with EtOAc, dried over anhydrous Na₂SO₄, filtered, and concentrated under reduced pressure to give a residue, which was purified by prep-TLC (SiO₂, PE/EtOAc) to afford methyl 3-((2-((S)-(((benzyloxy)carbonyl)amino) (cycloheptyl)methyl)imidazo[1,2-b]pyridazin-6-yl)methyl)-2-oxo-5-(trifluoromethyl)piperidine-3-carboxylate (60.0 mg, crude) as yellow solid. LCMS [M+H]⁺=616.2 m/z.

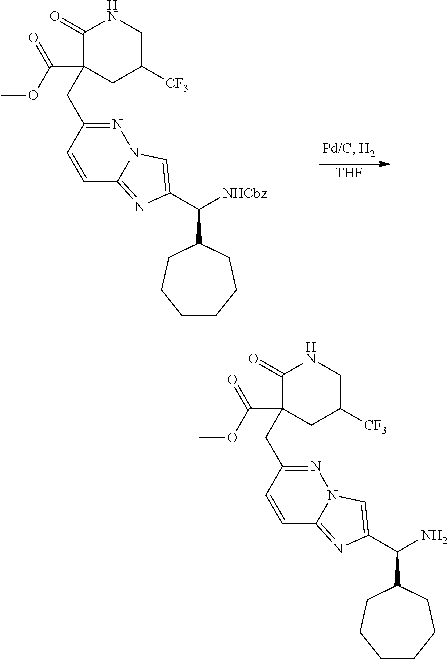 [SMILES: COC(=O)C1(Cc2ccc3nc([C@@H](NC(=O)OCc4ccccc4)C4CCCCCC4)cn3n2)CC(C(F)(F)F)CNC1=O ; COC(=O)C1(Cc2ccc3nc([C@@H](N)C4CCCCCC4)cn3n2)CC(C(F)(F)F)CNC1=O ; "THF"]

To a solution of methyl 3-((2-((S)-(((benzyloxy)carbonyl)amino) (cycloheptyl)methyl)imidazo[1,2-b]pyridazin-6-yl)methyl)-2-oxo-5-(trifluoromethyl)piperidine-3-carboxylate (60.0 mg, 97.4 mmol, 1.00 eq) in THF (3.00 mL) was added Pd/C (40.0 mg, 10% purity). The reaction mixture was stirred at 18° C. for 5 h under H₂ (15 psi). The reaction mixture was filtered through a pad of Celite and concentrated under reduced pressure to afford methyl 3-((2-((S)-amino(cycloheptyl)methyl)imidazo[1,2-b]pyridazin-6-yl)methyl)-2-oxo-5-(trifluoromethyl)piperidine-3-carboxylate (50.0 mg, crude) as a yellow solid. LCMS [M+H]⁺=482.3 m/z.

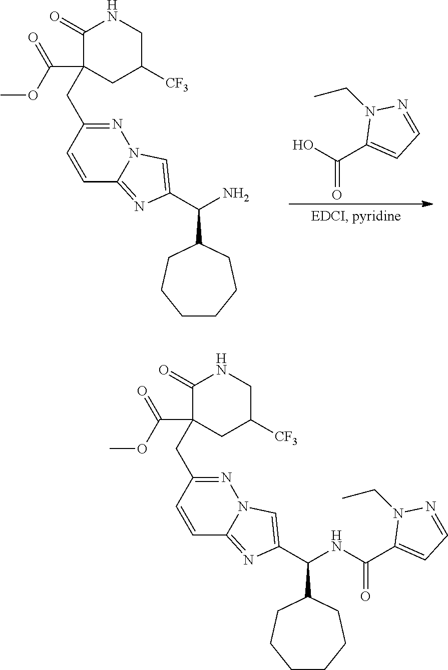

To a solution of methyl 3-((2-((S)-amino(cycloheptyl)methyl)imidazo[1,2-b]pyridazin-6-yl)methyl)-2-oxo-5-(trifluoromethyl)piperidine-3-carboxylate (40.0 mg, 83.0 mmol, 1.00 eq) and 1-ethyl-1H-pyrazole-5-carboxylic acid (13.9 mg, 99.6 mmol, 1.20 eq) in pyridine (1.00 mL) was added EDCI (63.7 mg, 332 mmol, 4.00 eq). The reaction mixture was stirred at 18° C. f or 2 h. The reaction mixture was diluted with water and extracted with EtOAc. The combined organic layers were dried over anhydrous Na₂SO₄, filtered, and concentrated under reduced pressure. The resulting residue was purified by prep-TLC (SiO₂, EtOAc/MeOH) to afford methyl 3-((2-((S)-cycloheptyl(1-ethyl-1H-pyrazole-5-carboxamido)methyl)imidazo[1,2-b]pyridazin-6-yl)methyl)-2-oxo-5-(trifluoromethyl)piperidine-3-carboxylate (45.0 mg, 74.5 mmol, 90% yield) as a yellow solid. LCMS [M+H]⁺=604.4 m/z.

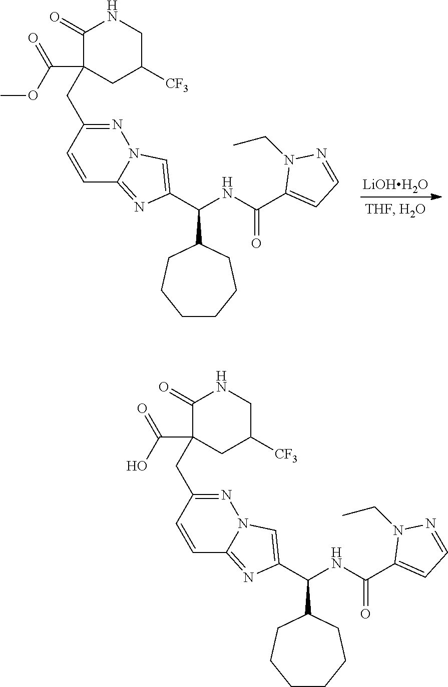

To a solution of methyl 3-((2-((S)-cycloheptyl(1-ethyl-1H-pyrazole-5-carboxamido)methyl)imidazo[1,2-b]pyridazin-6-yl)methyl)-2-oxo-5-(trifluoromethyl)piperidine-3-carboxylate (45.0 mg, 74.5 mmol, 1.00 eq) in THF (2.00 mL) and water (0.50 mL) was added LiOH·H₂O (9.38 mg, 223 mmol, 3.00 eq). The reaction mixture was stirred at 18° C. for 6 h. The reaction mixture was adjusted to pH=4 with a 1 M aqueous solution of HCl. The reaction mixture was concentrated under reduced pressure to afford 3-((2-((S)-cycloheptyl(1-ethyl-1H-pyrazole-5-carboxamido)methyl)imidazo[1,2-b]pyridazin-6-yl)methyl)-2-oxo-5-(trifluoromethyl)piperidine-3-carboxylic acid (40.0 mg, 67.8 mmol, 91% yield) as a white solid. LCMS [M+H]⁺=590.4 m/z.

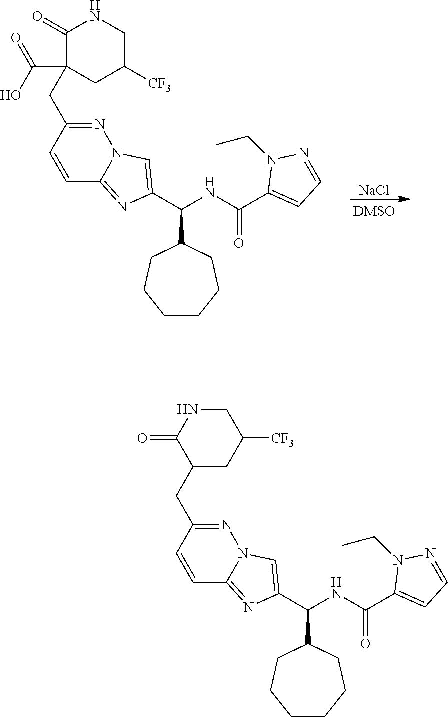

To a solution of 3-((2-((S)-cycloheptyl(1-ethyl-1H-pyrazole-5-carboxamido)methyl) imidazo[1,2-b]pyridazin-6-yl)methyl)-2-oxo-5-(trifluoromethyl)piperidine-3-carboxylic acid (40.0 mg, 67.8 mmol, 1.00 eq) in DMSO (1.00 mL) was added NaCl (11.8 mg, 203 mmol, 3.00 eq). The reaction mixture was stirred at 120° C. for 1 h. The reaction mixture was concentrated under reduced pressure to give a residue, which was purified by prep-HPLC (neutral condition; column: Phenomenex Luna C₁₈ (150 mm×25 mm, 10 mm); mobile phase: [water (HCOOH)-ACN]; B %: 41%-71%, 10 min) to afford N-((1S)-cycloheptyl(6-((2-oxo-5-(trifluoromethyl)piperidin-3-yl)methyl)imidazo[1,2-b]pyridazin-2-yl)methyl)-1-ethyl-1H-pyrazole-5-carboxamide (20.14 mg, 35.4 mmol, 52% yield) as a white solid. LCMS [M+H]⁺=546.3 m/z.

### Example 7: Preparation of Compound 4

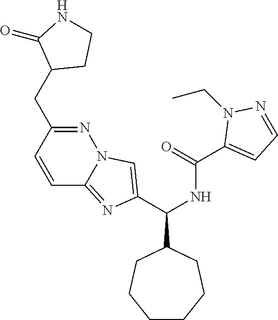 [SMILES: CCn1nccc1C(=O)N[C@H](c1cn2nc(CC3CCNC3=O)ccc2n1)C1CCCCCC1]

### N-((1S)-cycloheptyl(6-((2-oxopyrrolidin-3-yl)methyl)imidazo[1,2-b]pyridazin-2-yl)methyl)-1-ethyl-1H-pyrazole-5-carboxamide. (Compound 4)

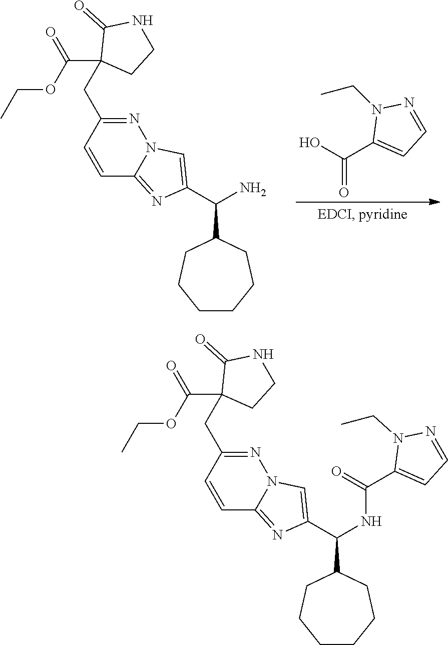

To a solution of 1-ethyl-1H-pyrazole-5-carboxylic acid (52.8 mg, 377 mmol, 1.20 eq) and ethyl 3-((2-((S)-amino(cycloheptyl)methyl)imidazo[1,2-b]pyridazin-6-yl)methyl)-2-oxopyrrolidine-3-carboxylate (130 mg, 314 mmol, 1.00 eq) in pyridine (6.00 mL) was added EDCl (180 mg, 94.14 mmol, 3.00 eq). The reaction mixture was stirred at RT for 3 h. The reaction mixture was diluted with water and extracted with DCM. The combined organic layers were washed with brine, dried over anhydrous Na₂SO₄, filtered, and concentrated under reduced pressure to afford ethyl 3-((2-((S)-cycloheptyl(1-ethyl-1H-pyrazole-5-carboxamido)methyl)imidazo[1,2-b]pyridazin-6-yl)methyl)-2-oxopyrrolidine-3-carboxylate (150 mg, crude) as a white solid.

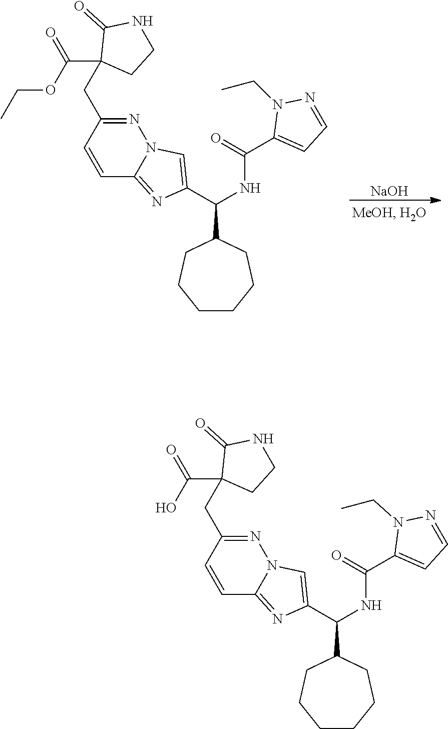 [SMILES: CCOC(=O)C1(Cc2ccc3nc([C@@H](NC(=O)c4ccnn4CC)C4CCCCCC4)cn3n2)CCNC1=O ; "NaOH" ; CCn1nccc1C(=O)N[C@H](c1cn2nc(CC3(C(=O)O)CCNC3=O)ccc2n1)C1CCCCCC1]

To a solution of ethyl 3-((2-((S)-cycloheptyl(1-ethyl-1H-pyrazole-5-carboxamido) methyl)imidazo[1,2-b]pyridazin-6-yl)methyl)-2-oxopyrrolidine-3-carboxylate (140 mg, 261 mmol, 1.00 eq) in MeOH (3.00 mL) and water (1.00 mL) was added NaOH (52.2 mg, 1.31 mmol, 5.00 eq). The reaction mixture was stirred at RT for 1 h. The reaction mixture was washed with EtOAc. The pH of the aqueous phase was adjusted to 3 with a 1 M aqueous solution of HCl and extracted with EtOAc. The combined organic layers were dried over anhydrous Na₂SO₄, filtered, and concentrated under reduced pressure to afford 3-((2-((S)-cycloheptyl(1-ethyl-1H-pyrazole-5-carboxamido)methyl)imidazo[1,2-b]pyridazin-6-yl)methyl)-2-oxopyrrolidine-3-carboxylic acid (100 mg, crude) as a white solid.

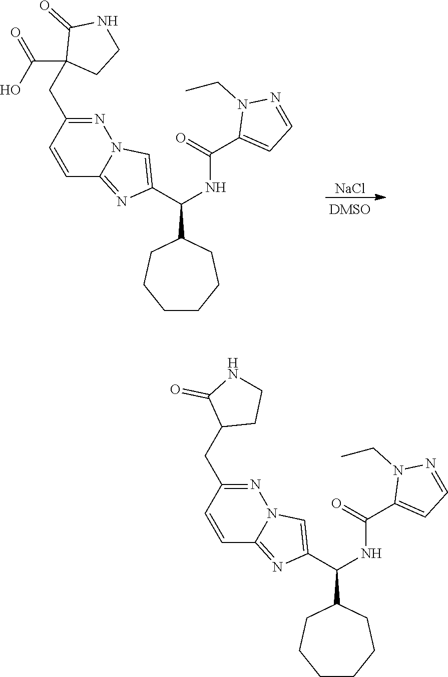 [SMILES: CCn1nccc1C(=O)N[C@H](c1cn2nc(CC3(C(=O)O)CCNC3=O)ccc2n1)C1CCCCCC1 ; "NaCl" ; CCn1nccc1C(=O)N[C@H](c1cn2nc(CC3CCNC3=O)ccc2n1)C1CCCCCC1]

To a solution of 3-((2-((S)-cycloheptyl(1-ethyl-1H-pyrazole-5-carboxamido)methyl)imidazo[1,2-b]pyridazin-6-yl)methyl)-2-oxopyrrolidine-3-carboxylic acid (100 mg, 197 mmol, 1.00 eq) in DMSO (8.00 mL) was added NaCl (34.5 mg, 591 mmol, 3.00 eq). The reaction mixture was stirred at 130° C. for 1 h. The reaction mixture was diluted with water and extracted with DCM. The combined organic layers were washed with brine, dried over anhydrous Na₂SO₄, filtered, and concentrated under reduced pressure. The resulting residue was purified by prep-HPLC (neutral condition; column: Waters Xbridge (150 mm×25 mm, 5 um); mobile phase: [water (NH₄OH)-ACN]; B %: 27%-54%, 9 min) to afford N—((1S)-cycloheptyl(6-((2-oxopyrrolidin-3-yl)methyl)imidazo[1,2-b]pyridazin-2-yl)methyl)-1-ethyl-1H-pyrazole-5-carboxamide (57.0 mg, crude) as a white solid. The title compound was isolated as the second eluting, single stereoisomer by chiral SFC purification (column: Daiel Chiralpak IG (250 mm×30 mm, 10 mm); mobile phase: [ACN/MeOH (0.1% NH₃H₂O)]; B %: 60%-60%, 3.6 min); (17.3 mg, 36.1 mmol, 31% yield) and was obtained as a white solid. LCMS [M+H]⁺=464.4 m/z.

### Example 8: Preparation of Compounds 5 and 6

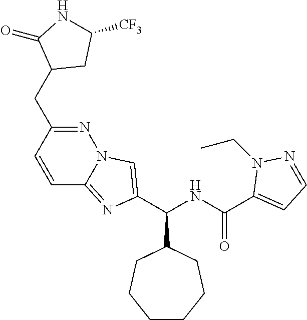 [SMILES: CCn1nccc1C(=O)N[C@H](c1cn2nc(CC3C[C@@H](C(F)(F)F)NC3=O)ccc2n1)C1CCCCCC1]

### N-((1S)-cycloheptyl(6-(((5S)-2-oxo-5-(trifluoromethyl)pyrrolidin-3-yl)methyl)imidazo[1,2-b]pyridazin-2-yl)methyl)-1-ethyl-1H-pyrazole-5-carboxamide. (Compounds 5 and 6)

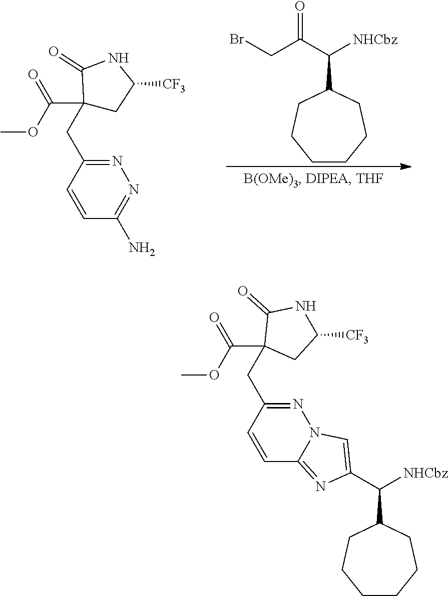 [SMILES: COC(=O)C1(Cc2ccc(N)nn2)C[C@@H](C(F)(F)F)NC1=O ; O=C(N[C@H](C(=O)CBr)C1CCCCCC1)OCc1ccccc1 ; COC(=O)C1(Cc2ccc3nc([C@@H](NC(=O)OCc4ccccc4)C4CCCCCC4)cn3n2)C[C@@H](C(F)(F)F)NC1=O]

To a solution of methyl (5S)-3-((6-aminopyridazin-3-yl)methyl)-2-oxo-5-(trifluoromethyl)pyrrolidine-3-carboxylate (300 mg, 694 mmol, 1.00 eq, TFA salt) in THF (5.00 mL) was added benzyl (S)-(3-bromo-1-cycloheptyl-2-oxopropyl)carbamate (Int. 3; 397 mg, 1.04 mmol, 1.50 eq), trimethyl borate (360 mg, 3.47 mmol, 391 mL, 5.00 eq), and DIEA (448 mg, 3.47 mmol, 604 mL, 5.00 eq). The reaction mixture was stirred at 80° C. for 2 h. The reaction mixture was diluted with water and extracted with EtOAc. The combined organic layers were washed with a saturated aqueous solution of NaCO₃, dried over anhydrous Na₂SO₄, filtered, and concentrated under reduced pressure. The resulting residue was purified by prep-TLC (SiO₂, DCM/MeOH) to afford methyl (5S)-3-((2-((S)-(((benzyloxy)carbonyl)amino)(cycloheptyl)methyl)imidazo[1,2-b]pyridazin-6-yl)methyl)-2-oxo-5-(trifluoromethyl)pyrrolidine-3-carboxylate (250 mg, 415 mmol, 60% yield) as a white solid. LCMS [M+H]⁺=602.3 m/z.

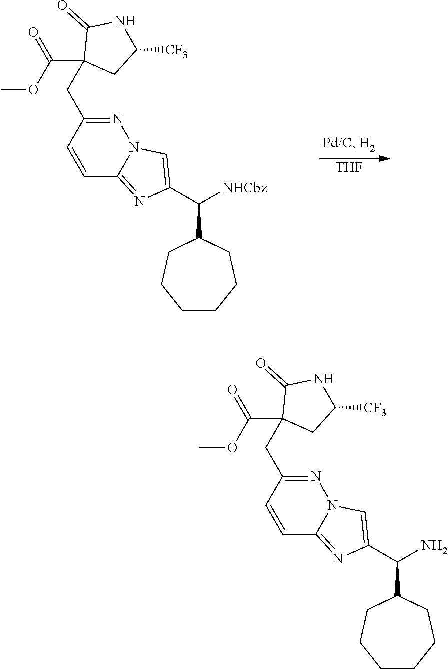 [SMILES: COC(=O)C1(Cc2ccc3nc([C@@H](NC(=O)OCc4ccccc4)C4CCCCCC4)cn3n2)C[C@@H](C(F)(F)F)NC1=O ; COC(=O)C1(Cc2ccc3nc([C@@H](N)C4CCCCCC4)cn3n2)C[C@@H](C(F)(F)F)NC1=O ; "THF"]

To a solution of methyl (5S)-3-((2-((S)-(((benzyloxy)carbonyl)amino)(cycloheptyl)methyl) imidazo[1,2-b]pyridazin-6-yl)methyl)-2-oxo-5-(trifluoromethyl)pyrrolidine-3-carboxylate (250 mg, 415 mmol, 1.00 eq) in THF (5.00 mL) was added Pd/C (25.0 mg, 10.0% purity) under N₂ atmosphere. The suspension was degassed and purged with H₂ (3 times). The reaction mixture was stirred at RT for 2 h under H₂ (15 psi). The reaction mixture was filtered through a pad of Celite with MeOH and concentrated under reduced pressure to afford methyl (5S)-3-((2-((S)-amino(cycloheptyl)methyl)imidazo[1,2-b]pyridazin-6-yl)methyl)-2-oxo-5-(trifluoromethyl) pyrrolidine-3-carboxylate (180 mg, 385 mmol, 93% yield) as a white solid. LCMS [M+H]⁺=468.4 m/z.

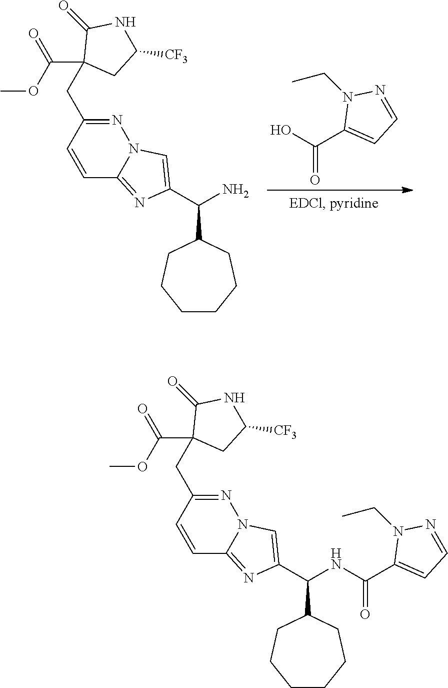

To a solution of methyl (5S)-3-((2-((S)-amino(cycloheptyl)methyl)imidazo[1,2-b]pyridazin-6-yl)methyl)-2-oxo-5-(trifluoromethyl)pyrrolidine-3-carboxylate (180 mg, 385 mmol, 1.00 eq) and 1-ethyl-1H-pyrazole-5-carboxylic acid (80.9 mg, 577 mmol, 1.50 eq) in pyridine (5.00 mL) was added EDCI (221 mg, 1.16 mmol, 3.00 eq). The reaction mixture was stirred at RT for 2 h. The reaction mixture was diluted with water and extracted with EtOAc. The combined organic layers were washed with a saturated aqueous solution of NaCO₃, dried over anhydrous Na₂SO₄, filtered, and concentrated under reduced pressure. The resulting residue was purified by prep-TLC (SiO₂, DCM/MeOH) to afford methyl (5S)-3-((2-((S)-cycloheptyl(1-ethyl-1H-pyrazole-5-carboxamido)methyl)imidazo[1,2-b]pyridazin-6-yl)methyl)-2-oxo-5-(trifluoromethyl)pyrrolidine-3-carboxylate (130 mg, 220 mmol, 57% yield) as a white solid. LCMS [M+H]⁺=590.3 m/z.

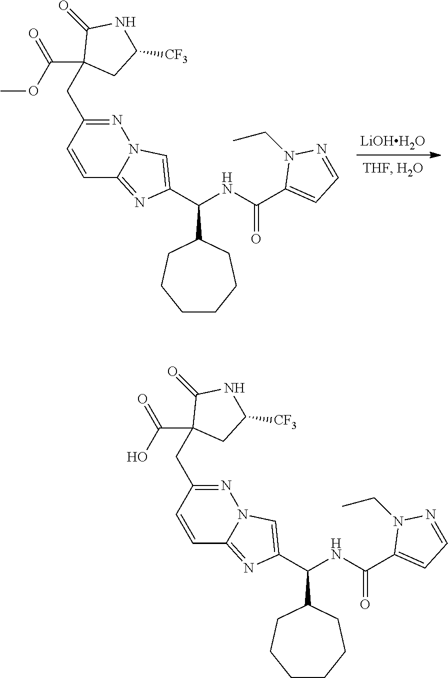 [SMILES: CCn1nccc1C(=O)N[C@H](c1cn2nc(CC3(C(=O)OC)C[C@@H](C(F)(F)F)NC3=O)ccc2n1)C1CCCCCC1 ; "LiOH·H2O" ; CCn1nccc1C(=O)N[C@H](c1cn2nc(CC3(C(=O)O)C[C@@H](C(F)(F)F)NC3=O)ccc2n1)C1CCCCCC1]

To a solution of methyl (5S)-3-((2-((S)-cycloheptyl(1-ethyl-1H-pyrazole-5-carboxamido)methyl)imidazo[1,2-b]pyridazin-6-yl)methyl)-2-oxo-5-(trifluoromethyl)pyrrolidine-3-carboxylate (130 mg, 220 mmol, 1.00 eq) in THF (4.00 mL) was added LiOH·H₂O (55.5 mg, 1.32 mmol, 6.00 eq) in water (1.00 mL) at 0° C. The reaction mixture was stirred at RT for 2 h. The reaction mixture was diluted with water, and a 1 M aqueous solution of HCl was added to adjust the pH to 2. The mixture was extracted with DCM. The combined organic layers were dried over anhydrous Na₂SO₄, filtered, and concentrated under reduced pressure to afford (5S)-3-((2-((S)-cycloheptyl(1-ethyl-1H-pyrazole-5-carboxamido)methyl)imidazo[1,2-b]pyridazin-6-yl)methyl)-2-oxo-5-(trifluoromethyl)pyrrolidine-3-carboxylic acid (120 mg, 208 mmol, 95% yield) as a white solid. LCMS [M+H]⁺=576.4 m/z.

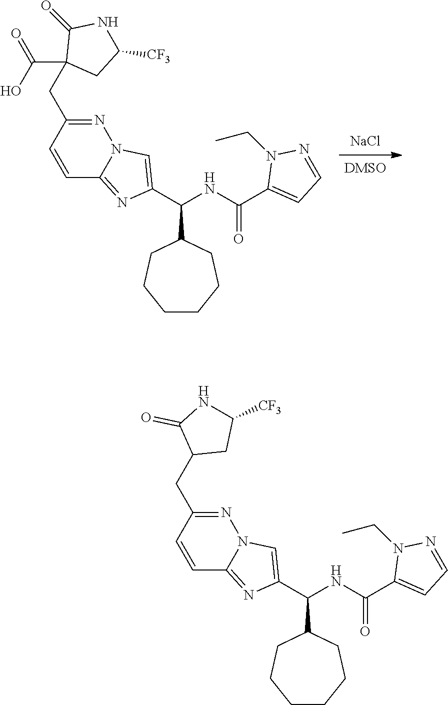

To a solution of 5S)-3-((2-((S)-cycloheptyl(1-ethyl-1H-pyrazole-5-carboxamido)methyl)imidazo[1,2-b]pyridazin-6-yl)methyl)-2-oxo-5-(trifluoromethyl)pyrrolidine-3-carboxylic acid (120 mg, 208 mmol, 1.00 eq) in DMSO (5.00 mL) was added NaCl (60.9 mg, 1.04 mmol, 5.00 eq). The reaction mixture was stirred at 120° C. for 2 h. The reaction mixture was diluted with water and extracted with EtOAc. The combined organic layers were washed with a saturated aqueous solution of NaCO₃, dried over anhydrous Na₂SO₄, filtered, and concentrated under reduced pressure. The resulting residue was purified by prep-TLC (SiO₂, DCM/MeOH) to afford N-((1S)-cycloheptyl(6-(((5S)-2-oxo-5-(trifluoromethyl)pyrrolidin-3-yl)methyl)imidazo[1,2-b]pyridazin-2-yl)methyl)-1-ethyl-1H-pyrazole-5-carboxamide. LCMS [M+H]⁺=532.4 m/z.

Compound 5: The title compound was isolated as the first eluting, single stereoisomer by chiral SFC purification (column: Daicel Chiralcel OJ-H (250 mm×30 mm, 5 mm); mobile phase: [0.1% NH₃H₂O in MeOH]; B %: 20%-20%, 2.0 min); (40.9 mg, 75.8 mmol, 566% yield) and was obtained as a white solid. LCMS [M+H]⁺=532.5 m/z.

Compound 6: The title compound was isolated as the second eluting, single stereoisomer by chiral SFC purification (column: Daicel Chiralcel OJ-H (250 mm×30 mm, 5 mm); mobile phase: [0.1% NH₃H₂O in MeOH]; B %: 20%-20%, 2.0 min); (14.6 mg, 26.9 mmol, 20% yield) as a white solid. LCMS [M+H]⁺=532.5 m/z.

### Example 9: Preparation of Compound 7

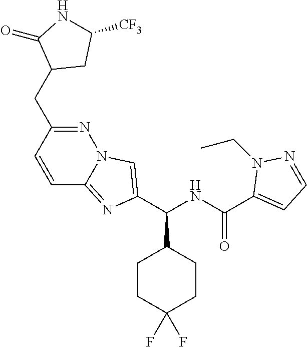 [SMILES: CCn1nccc1C(=O)N[C@H](c1cn2nc(CC3C[C@@H](C(F)(F)F)NC3=O)ccc2n1)C1CCC(F)(F)CC1]

### N-((1S)-(4,4-difluorocyclohexyl)(6-(((5S)-2-oxo-5-(trifluoromethyl)pyrrolidin-3-yl)methyl)imidazo[1,2-b]pyridazin-2-yl)methyl)-1-ethyl-1H-pyrazole-5-carboxamide. (Compound 7)

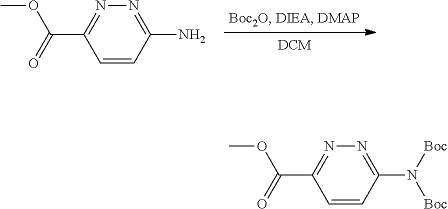 [SMILES: COC(=O)c1ccc(N)nn1 ; "Boc2O, DIEA, DMAP" ; COC(=O)c1ccc(N(C(=O)OC(C)(C)C)C(=O)OC(C)(C)C)nn1]

To a solution of methyl 6-aminopyridazine-3-carboxylate (10.0 g, 65.3 mmol, 1.00 eq) in DCM (100 mL) was added di-tert-butyl dicarbonate (42.7 g, 195 mmol, 45.0 mL, 3.00 eq), 4-dimethylaminopyridine (3.99 g, 32.6 mmol, 0.50 eq) and DIEA (33.7 g, 261 mmol, 45.5 mL, 4.00 eq) at 0° C. The reaction mixture was stirred at 15° C. for 12 h. The reaction mixture was concentrated under reduced pressure, and the obtained residue was purified by column chromatography (SiO₂, PE/EtOAc) to afford methyl 6-(bis(tert-butoxycarbonyl)amino) pyridazine-3-carboxylate (18 g, 50.94 mmol, 78% yield) as a white solid. ¹H NMR (400 MHz, CDCl₃) δ 8.21 (d, J=8.8 Hz, 1H), 7.85 (d, J=8.8 Hz, 1H), 4.08 (s, 3H), 1.50 (s, 18H). LCMS [M+H]⁺=354.3 m/z.

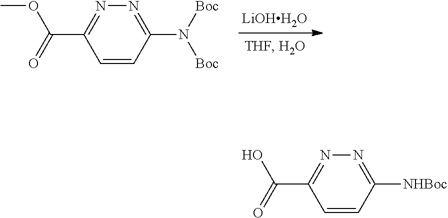 [SMILES: COC(=O)c1ccc(N(C(=O)OC(C)(C)C)C(=O)OC(C)(C)C)nn1 ; "LiOH·H2O" ; CC(C)(C)OC(=O)Nc1ccc(C(=O)O)nn1]

To a solution of methyl 6-(bis(tert-butoxycarbonyl)amino)pyridazine-3-carboxylate (19.5 g, 55 mmol, 1.00 eq) in THF (150 mL) and water (50.0 mL) was added LiOH·H₂O (4.63 g, 110 mmol, 2.00 eq) at 0° C. The reaction mixture was stirred at 20° C. for 12 h. The reaction mixture was diluted with a 1 M HCl to adjust pH to 5 and extracted with EtOAc. The combined organic layers were dried over anhydrous Na₂SO₄, filtered, and concentrated under reduced pressure to afford 6-((tert-butoxycarbonyl)amino)pyridazine-3-carboxylic acid (12.0 g, 50.1 mmol, 91% yield) as a white solid. ¹H NMR (400 MHz, CDCl₃) δ 8.55-8.48 (m, 1H), 8.41-8.20 (m, 1H), 8.18-7.95 (m, 1H), 1.58 (s, 9H). LCMS [M+Na]⁺=262.0 m/z.

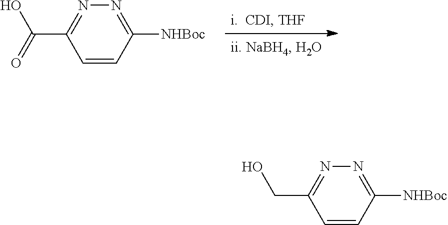 [SMILES: CC(C)(C)OC(=O)Nc1ccc(C(=O)O)nn1 ; "i.  CDI, THF" ; CC(C)(C)OC(=O)Nc1ccc(CO)nn1]

To a solution of 6-((tert-butoxycarbonyl)amino)pyridazine-3-carboxylic acid (12.0 g, 50.1 mmol, 1.00 eq) in THF (480 mL) was added 1,1′-carbonyldiimidazole (24.4 g, 150 mmol, 3.00 eq). The resulting mixture was stirred at 20° C. for 2 h, and then cooled down to 0° C. A solution of NaBH₄ (4.74 g, 125 mmol, 2.50 eq) in water (60.0 mL) was added dropwise. The reaction mixture was further stirred at 20° C. for 2 h. The reaction mixture was diluted with a saturated aqueous solution of NH₄Cl at 0° C. and extracted with EtOAc. The combined organic layers were dried over anhydrous Na₂SO₄, filtered, and concentrated under reduced pressure. The resulting residue was purified by column chromatography (SiO₂, PE/EtOAc) to afford tert-butyl (6-(hydroxymethyl)pyridazin-3-yl)carbamate (5.00 g, 22.2 mmol, 44% yield) as a white solid. ¹H NMR (400 MHz, CDCl₃) δ 8.25 (d, J=9.2 Hz, 1H), 7.67 (s, 1H), 7.49 (d, J=9.2 Hz, 1H), 4.91 (s, 2H), 1.55 (s, 9H). LCMS [M+H]⁺=226.1 m/z.

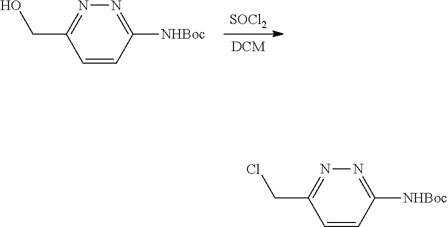 [SMILES: CC(C)(C)OC(=O)Nc1ccc(CO)nn1 ; "SOCl2" ; CC(C)(C)OC(=O)Nc1ccc(CCl)nn1]

A solution of tert-butyl (6-(hydroxymethyl)pyridazin-3-yl)carbamate (3.00 g, 13.3 mmol, 1.00 eq) in DCM (30.0 mL) and thionyl chloride (3.17 g, 26.6 mmol, 1.93 mL, 2.00 eq) was stirred at 0° C. for 1 h. The reaction mixture was concentrated under reduced pressure to afford tert-butyl (6-(chloromethyl)pyridazin-3-yl)carbamate (3.00 g, 12.3 mmol, 92% yield) as a yellow solid.

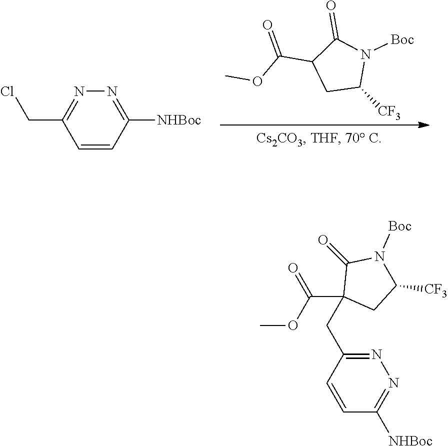 [SMILES: CC(C)(C)OC(=O)Nc1ccc(CCl)nn1 ; COC(=O)C1C[C@@H](C(F)(F)F)N(C(=O)OC(C)(C)C)C1=O ; COC(=O)C1(Cc2ccc(NC(=O)OC(C)(C)C)nn2)C[C@@H](C(F)(F)F)N(C(=O)OC(C)(C)C)C1=O]

To a solution of tert-butyl (6-(chloromethyl)pyridazin-3-yl)carbamate (1.00 g, 4.10 mmol, 1.00 eq) and 1-(tert-butyl) 3-methyl (5S)-2-oxo-5-(trifluoromethyl)pyrrolidine-1,3-dicarboxylate (2.11 g, 6.77 mmol, 1.10 eq) in DMF (10.0 mL) was added Cs₂CO₃ (4.01 g, 12.3 mmol, 2.00 eq). The reaction mixture was stirred at 55° C. for 2 h. The reaction mixture was diluted with a saturated aqueous solution of NaCO₃, extracted with EtOAc, dried over anhydrous Na₂SO₄, filtered, and concentrated under reduced pressure. The resulting residue was purified by column chromatography (SiO₂, PE/EtOAc) to afford 1-(tert-butyl) 3-methyl (5S)-3-((6-((tert-butoxycarbonyl)amino)pyridazin-3-yl)methyl)-2-oxo-5-(trifluoromethyl)pyrrolidine-1,3-dicarboxylate (1.30 g, 2.31 mmol, 61% yield) as a white solid. LCMS [M+H]⁺=519.3 m/z.

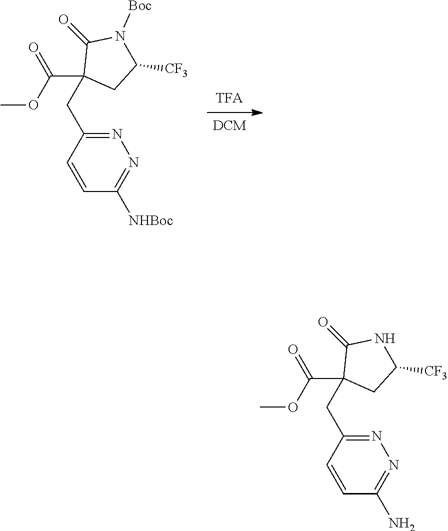 [SMILES: COC(=O)C1(Cc2ccc(NC(=O)OC(C)(C)C)nn2)C[C@@H](C(F)(F)F)N(C(=O)OC(C)(C)C)C1=O ; "TFA" ; COC(=O)C1(Cc2ccc(N)nn2)C[C@@H](C(F)(F)F)NC1=O]

A solution of 1-(tert-butyl)₃-methyl (5S)-3-((6-((tert-butoxycarbonyl)amino)pyridazin-3-yl)methyl)-2-oxo-5-(trifluoromethyl)pyrrolidine-1,3-dicarboxylate (1.00 g, 1.93 mmol, 1.00 eq) in DCM (10.0 mL) and TFA (1.10 g, 9.64 mmol, 714 mL, 5.00 eq) was stirred at 15° C. for 2 h. The reaction mixture was concentrated under reduced pressure to afford methyl (5S)-3-((6-aminopyridazin-3-yl)methyl)-2-oxo-5-(trifluoromethyl)pyrrolidine-3-carboxylate (810 mg, 1.87 mmol, 97% yield, TFA salt) as a yellow oil. LCMS [M+H]⁺=319.1 m/z.

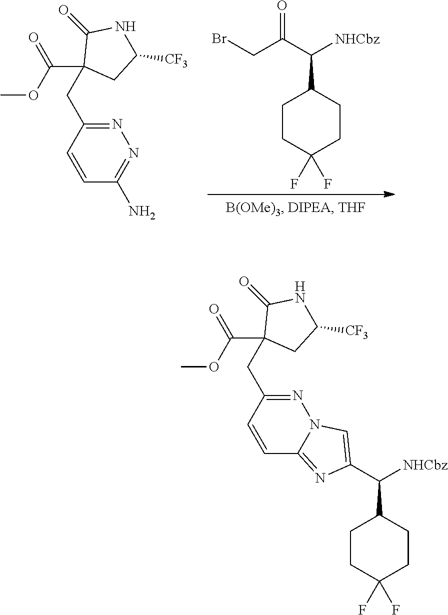 [SMILES: COC(=O)C1(Cc2ccc(N)nn2)C[C@@H](C(F)(F)F)NC1=O ; O=C(N[C@H](C(=O)CBr)C1CCC(F)(F)CC1)OCc1ccccc1 ; COC(=O)C1(Cc2ccc3nc([C@@H](NC(=O)OCc4ccccc4)C4CCC(F)(F)CC4)cn3n2)C[C@@H](C(F)(F)F)NC1=O]

To a solution of (5S)-3-((6-aminopyridazin-3-yl)methyl)-2-oxo-5-(trifluoromethyl) pyrrolidine-3-carboxylate (270 mg, 624 mmol, 1.00 eq, TFA salt) and benzyl (S)-(3-bromo-1-(4,4-difluorocyclohexyl)-2-oxopropyl)carbamate (Int. 2; 379 mg, 937 mmol, 1.50 eq) in THF (2.00 mL) was added DIEA (403 mg, 3.12 mmol, 544 mL, 5.00 eq) and trimethyl borate (324 mg 3.12 mmol, 352 mL, 5.00 eq). The reaction mixture was stirred at 80° C. for 2 h. The reaction mixture was diluted with a saturated aqueous solution of NaCO₃, extracted with EtOAc, dried over anhydrous Na₂SO₄, filtered, and concentrated under reduced pressure. The resulting residue was purified by prep-TLC (SiO₂, DCM/MeOH) to afford methyl (5S)-3-((2-((S)-(((benzyloxy)carbonyl)amino)(4,4-difluorocyclohexyl)methyl)imidazo[1,2-b]pyridazin-6-yl)methyl)-2-oxo-5-(trifluoromethyl)pyrrolidine-3-carboxylate (200 mg, 481 mmol, 51% yield) as a yellow solid. LCMS [M+H]⁺=624.2 m/z.

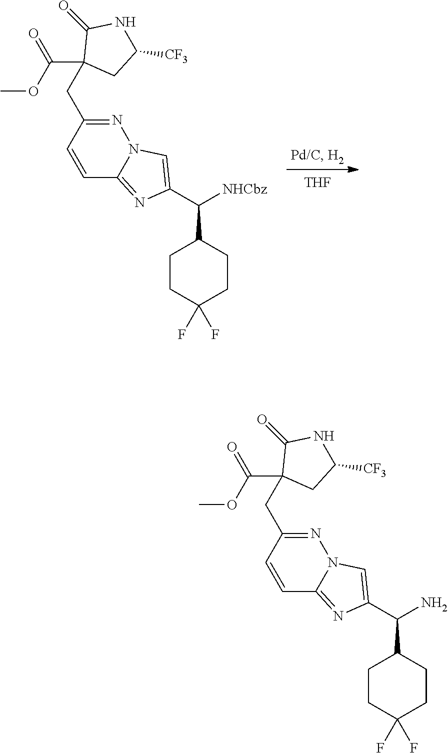 [SMILES: COC(=O)C1(Cc2ccc3nc([C@@H](NC(=O)OCc4ccccc4)C4CCC(F)(F)CC4)cn3n2)C[C@@H](C(F)(F)F)NC1=O ; "Pd/C, H2" ; "THF" ; COC(=O)C1(Cc2ccc3nc([C@@H](N)C4CCC(F)(F)CC4)cn3n2)C[C@@H](C(F)(F)F)NC1=O]

To a solution of methyl (5S)-3-((2-((S)-(((benzyloxy)carbonyl)amino)(4,4-difluorocyclohexyl)methyl)imidazo[1,2-b]pyridazin-6-yl)methyl)-2-oxo-5-(trifluoromethyl)pyrrolidine-3-carboxylate (200 mg, 481 mmol, 1.00 eq) in THE (10.0 mL) was added Pd/C (50.0 mg, 10% purity) under N₂. The suspension was degassed under vacuum and purged with H₂ (3 times). The reaction mixture was stirred at 15° C. for 12 h under H₂ (15 psi). The reaction mixture was filtered through a pad of Celite and concentrated under reduced pressure to afford methyl (5S)-3-((2-((S)-amino(4,4-difluorocyclohexyl)methyl)imidazo[1,2-b]pyridazin-6-yl)methyl)-2-oxo-5-(trifluoromethyl)pyrrolidine-3-carboxylate (130 mg, 265 mmol, 83% yield) as a yellow solid. LCMS [M+H]⁺=490.3 m/z.

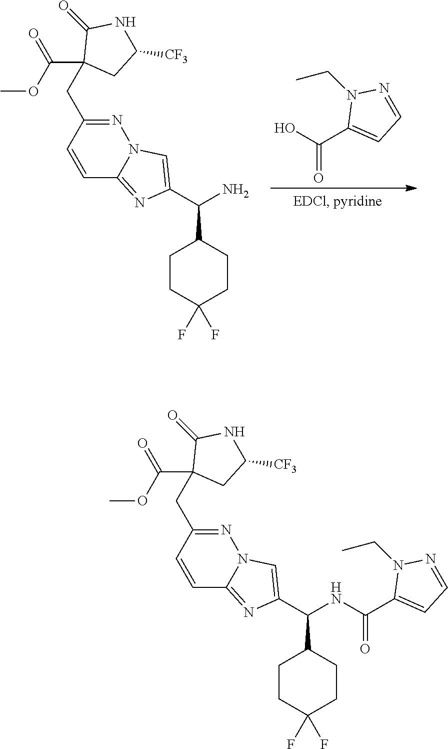

To a solution of methyl (5S)-3-((2-((S)-amino(4,4-difluorocyclohexyl)methyl)imidazo[1,2-b]pyridazin-6-yl)methyl)-2-oxo-5-(trifluoromethyl)pyrrolidine-3-carboxylate (130 mg, 265 mmol, 1.00 eq) and 1-ethyl-1H-pyrazole-5-carboxylic acid (55.8 mg, 398 mmol, 1.50 eq) in pyridine (5.00 mL) was added EDCI (101 mg, 531 mmol, 2.00 eq). The reaction mixture was stirred at 15° C. for 2 h. The reaction mixture was diluted with a saturated aqueous solution of NaCO₃ and extracted with EtOAc. The combined organic layers were dried over anhydrous Na₂SO₄, filtered, and concentrated under reduced pressure to give a residue, which was purified by prep-TLC (SiO₂, DCM/MeOH) to afford methyl (5S)-3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(trifluoromethyl)pyrrolidine-3-carboxylate (120 mg, 196 mmol, 74% yield) as a yellow solid. LCMS [M+H]⁺=612.3 m/z.

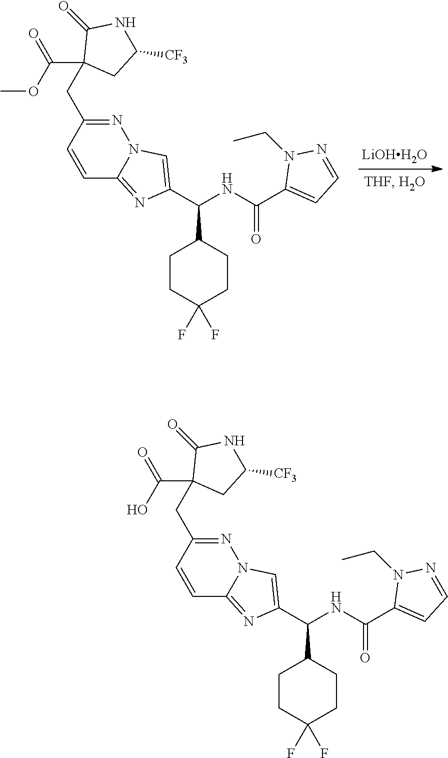

To a solution of methyl (5S)-3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(trifluoromethyl)pyrrolidine-3-carboxylate (120 mg, 196 mmol, 1.00 eq) in THF (5.00 mL) and water (5.00 mL) was added LiOH·H₂O (16.4 mg, 392 mmol, 2.00 eq) at 0° C. The reaction mixture was stirred at 15° C. for 2 h. The reaction mixture was acidified to pH=4 with a 1 M aqueous solution of HCl before extracting with EtOAc. The organic layers were dried over anhydrous Na₂SO₄, filtered, and concentrated under reduced pressure to afford (5S)-3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(trifluoromethyl)pyrrolidine-3-carboxylic acid (100 mg, 167 mmol, 85% yield) as a yellow solid. LCMS [M+H]⁺=598.3 m/z.

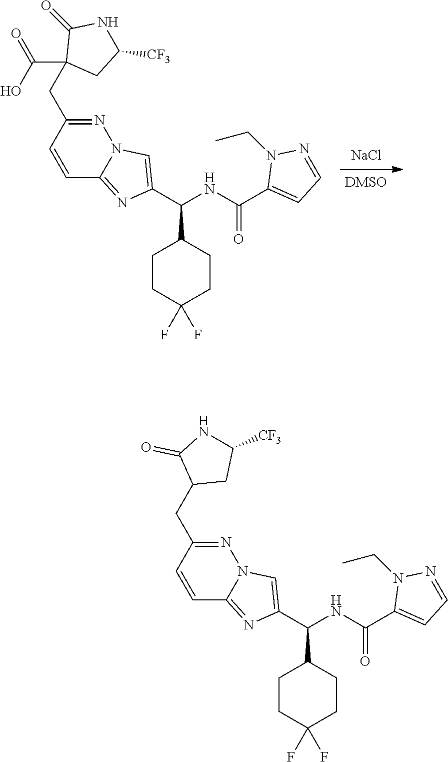

To a solution of (5S)-3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(trifluoromethyl)pyrrolidine-3-carboxylic acid (100 mg, 167 mmol, 1.00 eq) in DMSO (5.00 mL) was added NaCl (19.5 mg, 334 mmol, 2.00 eq). The reaction mixture was stirred at 120° C. for 2 h. The reaction mixture was diluted with water, extracted with EtOAc, dried over anhydrous Na₂SO₄, filtered, and concentrated under reduced pressure to give a residue, which was purified by prep-TLC (SiO₂, DCM/MeOH) to afford N-((1S)-(4,4-difluorocyclohexyl)(6-(((5S)-2-oxo-5-(trifluoromethyl) pyrrolidin-3-yl)methyl)imidazo[1,2-b]pyridazin-2-yl)methyl)-1-ethyl-1H-pyrazole-5-carboxamide. The title compound was isolated as the second eluting, single stereoisomer by chiral SFC purification (column: Daicel Chiralcel OJ (250 mm×50 mm, 10 mm); mobile phase: [0.1% NH₃H₂O in IPA]; B %: 20%-20%, 2.5 min); (17.53 mg, 28.79 mmol, 53% yield) and was obtained as a white solid. LCMS [M+H]⁺=554.3 m/z.

### Example 10: Preparation of Compound 8

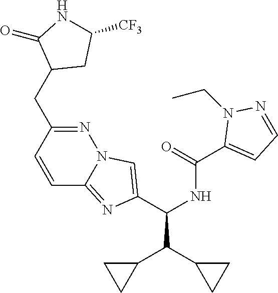 [SMILES: CCn1nccc1C(=O)N[C@H](c1cn2nc(CC3C[C@@H](C(F)(F)F)NC3=O)ccc2n1)C(C1CC1)C1CC1]

### N-((1 S)-2,2-dicyclopropyl-1-(6-(((5S)-2-oxo-5-(trifluoromethyl)pyrrolidin-3-yl)methyl)imidazo[1,2-b]pyridazin-2-yl)ethyl)-1-ethyl-1H-pyrazole-5-carboxamide. (Compound 8)

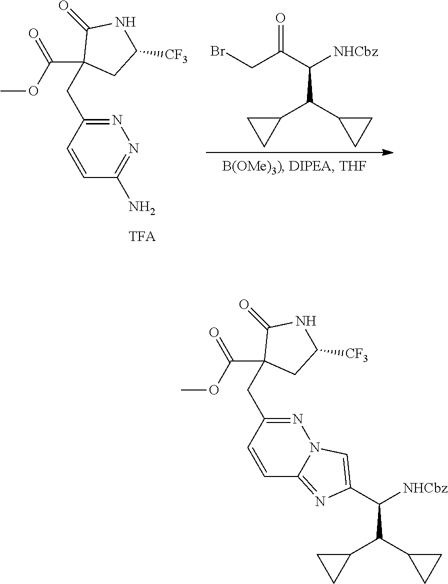 [SMILES: COC(=O)C1(Cc2ccc(N)nn2)C[C@@H](C(F)(F)F)NC1=O ; O=C(N[C@H](C(=O)CBr)C(C1CC1)C1CC1)OCc1ccccc1 ; COC(=O)C1(Cc2ccc3nc([C@@H](NC(=O)OCc4ccccc4)C(C4CC4)C4CC4)cn3n2)C[C@@H](C(F)(F)F)NC1=O]

To a solution of methyl (5S)-3-((6-aminopyridazin-3-yl)methyl)-2-oxo-5-(trifluoromethyl)pyrrolidine-3-carboxylate (300 mg, 694 μmol, 1.00 eq, TFA salt) in THE (5 mL) was added benzyl (S)-(4-bromo-1,1-dicyclopropyl-3-oxobutan-2-yl)carbamate (Int. 1; 395 mg, 1.04 mmol, 1.50 eq), B(OMe)₃ (360 mg, 3.47 mmol, 391 μL, 5.00 eq), and DIPEA (448 mg, 3.47 mmol, 604 μL, 5.00 eq). The mixture was stirred at 80° C. for 2 h. The reaction mixture was diluted with H₂O and extracted with EtOAc. The combined organic layers were washed with saturated aqueous NaHCO₃, dried over Na₂SO₄, filtered, and concentrated under reduced pressure. The residue was purified by prep-TLC (SiO₂, DCM:MeOH=10:1) to afford methyl (5 S)-3-((2-((S)-1-(((benzyloxy)carbonyl)amino)-2,2-dicyclopropylethyl)imidazo[1,2-b]pyridazin-6-yl)methyl)-2-oxo-5-(trifluoromethyl)pyrrolidine-3-carboxylate (250 mg, 416 μmol, 60% yield) as a white solid. LCMS [M+H]⁺=600.3 m/z.

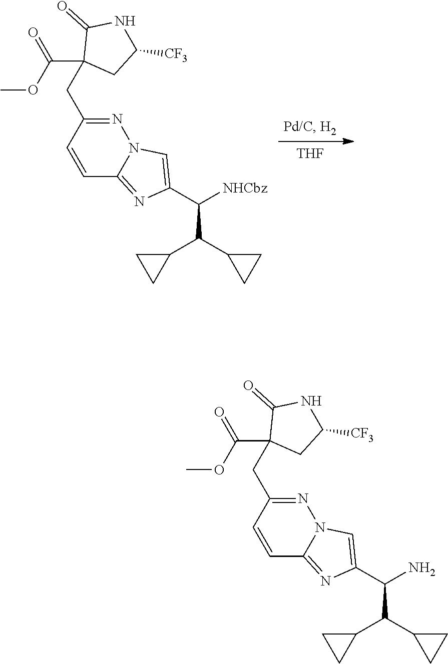 [SMILES: COC(=O)C1(Cc2ccc3nc([C@@H](NC(=O)OCc4ccccc4)C(C4CC4)C4CC4)cn3n2)C[C@@H](C(F)(F)F)NC1=O ; COC(=O)C1(Cc2ccc3nc([C@@H](N)C(C4CC4)C4CC4)cn3n2)C[C@@H](C(F)(F)F)NC1=O ; "THF"]

To a solution of methyl (5S)-3-((2-((S)-1-(((benzyloxy)carbonyl)amino)-2,2-dicyclopropylethyl)imidazo[1,2-b]pyridazin-6-yl)methyl)-2-oxo-5-(trifluoromethyl)pyrrolidine-3-carboxylate (250 mg, 416 μmol, 1.00 eq) in THF (5.00 mL) was added Pd/C (25.0 mg, 10.0% purity) under N₂ atmosphere. The suspension was degassed and purged with H₂ 3 times. The mixture was stirred under H₂ (15 psi) at RT for 2 h. The reaction mixture was filtered, the filter cake was washed with MeOH, and the filtrate was concentrated under reduced pressure to give methyl (5S)-3-((2-((S)-1-amino-2,2-dicyclopropylethyl)imidazo[1,2-b]pyridazin-6-yl)methyl)-2-oxo-5-(trifluoromethyl)pyrrolidine-3-carboxylate (150 mg, 322 μmol, 77% yield) as a white solid.

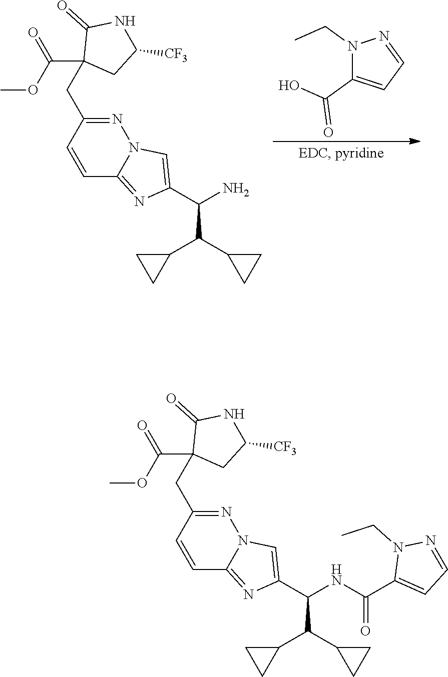

To a solution of methyl (5S)-3-((2-((S)-1-amino-2,2-dicyclopropylethyl)imidazo[1,2-b]pyridazin-6-yl)methyl)-2-oxo-5-(trifluoromethyl)pyrrolidine-3-carboxylate (150 mg, 322 μmol, 1.00 eq) and 1-ethyl-1H-pyrazole-5-carboxylic acid (67.7 mg, 483 μmol, 1.50 eq) in pyridine (5.00 mL) was added EDCI (185 mg, 966 μmol, 3.00 eq). The mixture was stirred at RT for 2 h. The reaction mixture was diluted with H₂O and extracted with EtOAc. The combined organic layers were washed with saturated aqueous NaHCO₃, dried over Na₂SO₄, filtered, and concentrated under reduced pressure to give a residue. The residue was purified by prep-TLC (SiO₂, DCM:MeOH=10:1) to afford methyl (5S)-3-((2-((S)-2,2-dicyclopropyl-1-(1-ethyl-1H-pyrazole-5-carboxamido)ethyl)imidazo[1,2-b]pyridazin-6-yl)methyl)-2-oxo-5-(trifluoromethyl)pyrrolidine-3-carboxylate (130 mg, 221 μmol, 69% yield) as a white solid. LCMS [M+H]⁺=588.3 m/z.

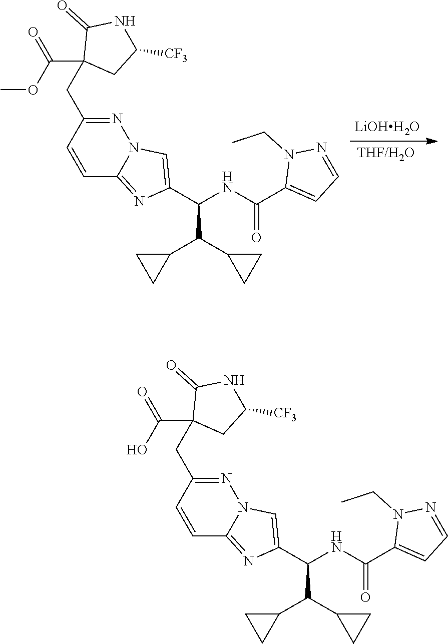

To a solution of methyl (5S)-3-((2-((S)-2,2-dicyclopropyl-1-(1-ethyl-1H-pyrazole-5-carboxamido)ethyl)imidazo[1,2-b]pyridazin-6-yl)methyl)-2-oxo-5-(trifluoromethyl)pyrrolidine-3-carboxylate (130 mg, 221 μmol, 1.00 eq) in THF (4.00 mL) was added LiOH·H₂O (55.7 mg, 1.33 mmol, 6.00 eq) in H₂O (1.00 mL) at 0° C. The mixture was stirred at RT for 1 h. The reaction mixture was diluted with H₂O, and 1 M HCl was added to adjust pH to 2. The mixture was then extracted with DCM, and the combined organic layers were dried over Na₂SO₄, filtered, and concentrated under reduced pressure to give (5S)-3-((2-((S)-2,2-dicyclopropyl-1-(1-ethyl-1H-pyrazole-5-carboxamido)ethyl)imidazo[1,2-b]pyridazin-6-yl)methyl)-2-oxo-5-(trifluoromethyl)pyrrolidine-3-carboxylic acid (120 mg, 209 μmol, 95% yield) as a white solid. LCMS [M+H]⁺=574.4 m/z.

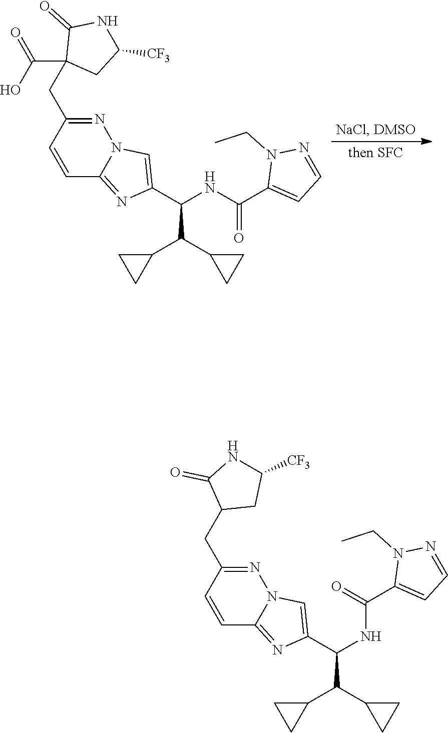

To a solution of (5S)-3-((2-((S)-2,2-dicyclopropyl-1-(1-ethyl-1H-pyrazole-5-carboxamido)ethyl)imidazo[1,2-b]pyridazin-6-yl)methyl)-2-oxo-5-(trifluoromethyl)pyrrolidine-3-carboxylic acid (120 mg, 209 μmol, 1.00 eq) in DMSO (2.00 mL) was added NaCl (61.1 mg 1.05 mmol, 5.00 eq). The mixture was stirred at 120° C. for 2 h. The reaction mixture was diluted with H₂O and extracted with EtOAc. The combined organic layers were washed with saturated aqueous NaHCO₃, dried over Na₂SO₄, filtered, and concentrated under reduced pressure. The residue was purified by prep-TLC (SiO₂, DCM:MeOH=10:1) to afford N-((1S)-2,2-dicyclopropyl-1-(6-(((5S)-2-oxo-5-(trifluoromethyl)pyrrolidin-3-yl)methyl)imidazo[1,2-b]pyridazin-2-yl)ethyl)-1-ethyl-1H-pyrazole-5-carboxamide as a mixture of two isomers (87.1 mg, 159 μmol, 76% yield) which was obtained as a white solid. The mixture of diastereomers was then purified by SFC (column: DAICEL CHIRALCEL OJ-H (250 mm*30 mm, 5 μm); mobile phase: [0.1% NH₃/H₂O EtOH]; B %:20%-20%, 2.2 min, Rt=0.865, 0.991 min). The second eluting single isomer (47.4 mg, 85.8 μmol, 54% yield) was obtained as a white solid. LCMS [M+H]⁺=530.4 m/z.

### Example 11: Preparation of Compounds 9 and 10

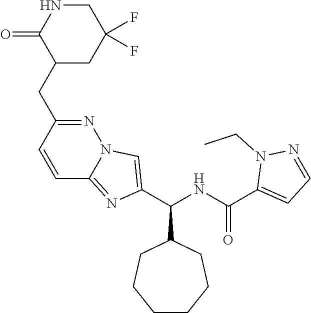 [SMILES: CCn1nccc1C(=O)N[C@H](c1cn2nc(CC3CC(F)(F)CNC3=O)ccc2n1)C1CCCCCC1]

### N-((1 S)-cycloheptyl(6-((5,5-difluoro-2-oxopiperidin-3-yl)methyl)imidazo[1,2-b]pyridazin-2-yl)methyl)-1-ethyl-1H-pyrazole-5-carboxamide. (Compounds 9 and 10)

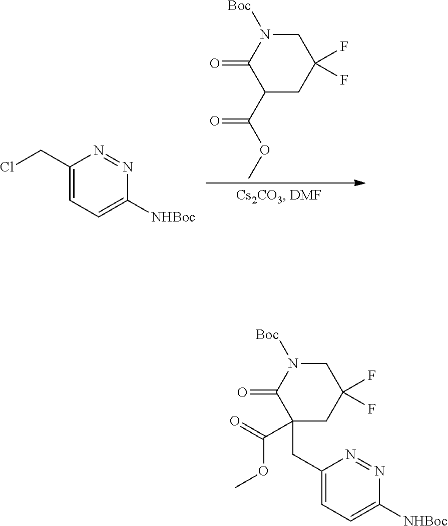 [SMILES: CC(C)(C)OC(=O)Nc1ccc(CCl)nn1 ; COC(=O)C1CC(F)(F)CN(C(=O)OC(C)(C)C)C1=O ; COC(=O)C1(Cc2ccc(NC(=O)OC(C)(C)C)nn2)CC(F)(F)CN(C(=O)OC(C)(C)C)C1=O]

To a solution of tert-butyl(6-(chloromethyl)pyridazin-3-yl)carbamate (0.200 g, 821 μmol, 1.00 eq) in DMF (4.00 mL) was added Cs₂CO₃ (535 mg, 1.64 mmol, 2.00 eq) and 1-(tert-butyl) 3-methyl 5,5-difluoro-2-oxopiperidine-1,3-dicarboxylate (289 mg, 985 μmol, 1.20 eq). The mixture was stirred at 70° C. for 2 h. The reaction mixture was diluted with H₂O and extracted with EtOAc. The combined organic layers were dried over Na₂SO₄, filtered, and concentrated under reduced pressure to give a residue. The residue was purified by prep-TLC (SiO₂, PE/EtOAc=1:1) to afford 1-(tert-butyl) 3-methyl 3-((6-((tert-butoxycarbonyl)amino)pyridazin-3-yl)methyl)-5,5-difluoro-2-oxopiperidine-1,3-dicarboxylate (0.220 g, 440 μmol, 54% yield) as a white solid. LCMS [M+H]⁺=501.3 m/z.

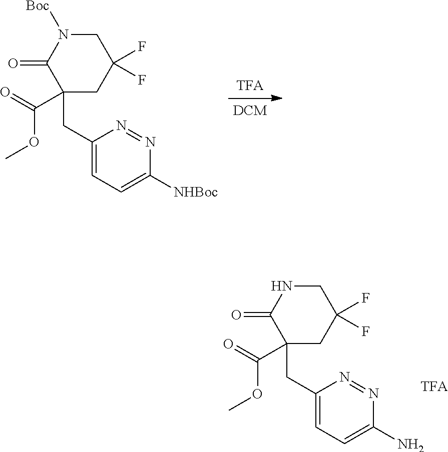 [SMILES: COC(=O)C1(Cc2ccc(NC(=O)OC(C)(C)C)nn2)CC(F)(F)CN(C(=O)OC(C)(C)C)C1=O ; COC(=O)C1(Cc2ccc(N)nn2)CC(F)(F)CNC1=O]

To a solution of 1-(tert-butyl) 3-methyl 3-((6-((tert-butoxycarbonyl)amino)pyridazin-3-yl)methyl)-5,5-difluoro-2-oxopiperidine-1,3-dicarboxylate (0.200 g, 400 μmol, 1.00 eq) in DCM (3.00 mL) was added TFA (456 mg, 4.00 mmol, 296 μL, 10.0 eq). The mixture was stirred at 15° C. for 3 h. The reaction mixture was concentrated under reduced pressure to give methyl 3-((6-aminopyridazin-3-yl)methyl)-5,5-difluoro-2-oxopiperidine-3-carboxylate (0.160 g, 386 μmol, 97% yield, TFA salt) as a yellow oil, which was used directly in the next step. LCMS [M+H]⁺=301.1 m/z.

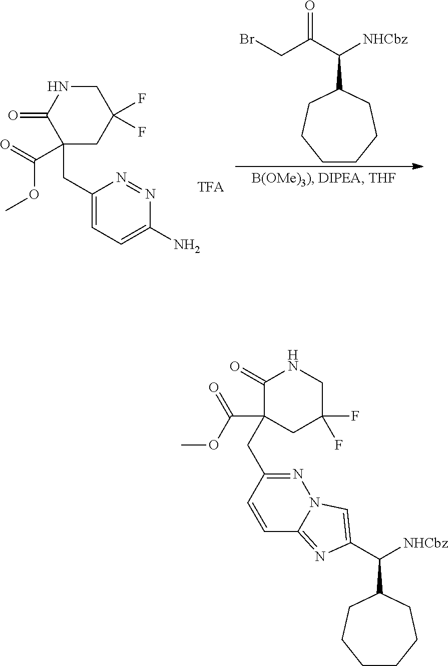 [SMILES: COC(=O)C1(Cc2ccc(N)nn2)CC(F)(F)CNC1=O ; O=C(N[C@H](C(=O)CBr)C1CCCCCC1)OCc1ccccc1 ; COC(=O)C1(Cc2ccc3nc([C@@H](NC(=O)OCc4ccccc4)C4CCCCCC4)cn3n2)CC(F)(F)CNC1=O]

To a solution of methyl 3-((6-aminopyridazin-3-yl)methyl)-5,5-difluoro-2-oxopiperidine-3-carboxylate (0.160 g, 386 μmol, 1.00 eq, TFA salt) and benzyl (S)-(3-bromo-1-cycloheptyl-2-oxopropyl)carbamate (Int. 3; 221 mg, 579 μmol, 1.50 eq) in THF (5.00 mL) was added B(OMe)₃ (201 mg, 1.93 mmol, 218 μL, 5.00 eq) and DIPEA (399 mg, 3.09 mmol, 538 μL, 8.00 eq). The mixture was stirred at 70° C. for 1 h. The reaction mixture was diluted with H₂O and extracted with EtOAc. The combined organic layers were dried over Na₂SO₄, filtered, and concentrated under reduced pressure. The residue was purified by prep-TLC (SiO₂, DCM:MeOH=10:1) to afford methyl 3-((2-((S)-(((benzyloxy)carbonyl)amino)(cycloheptyl)methyl)imidazo[1,2-b]pyridazin-6-yl)methyl)-5,5-difluoro-2-oxopiperidine-3-carboxylate (0.200 g, 343 μmol, 89% yield) as a yellow solid. LCMS [M+H]⁺=584.2 m/z.

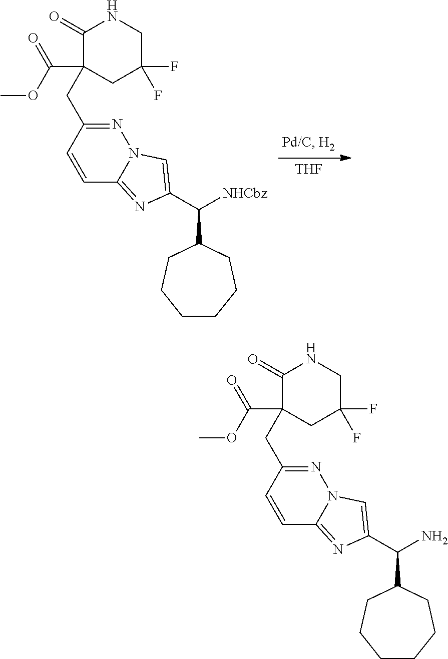 [SMILES: COC(=O)C1(Cc2ccc3nc([C@@H](NC(=O)OCc4ccccc4)C4CCCCCC4)cn3n2)CC(F)(F)CNC1=O ; "THF" ; COC(=O)C1(Cc2ccc3nc([C@@H](N)C4CCCCCC4)cn3n2)CC(F)(F)CNC1=O]

A mixture of methyl 3-((2-((S)-(((benzyloxy)carbonyl)amino) (cycloheptyl)methyl)imidazo[1,2-b]pyridazin-6-yl)methyl)-5,5-difluoro-2-oxopiperidine-3-carboxylate (0.150 g, 257 μmol, 1.00 eq) and Pd/C (0.100 g, 10.0% purity) in THE (3.00 mL) was degassed and purged with H₂ (15 psi) 3 times, then stirred at 15° C. for 2 h under H₂ (15 psi) atmosphere. The reaction mixture was filtered, and the filtrate was concentrated under reduced pressure to give methyl 3-((2-((S)-amino(cycloheptyl)methyl)imidazo[1,2-b]pyridazin-6-yl)methyl)-5,5-difluoro-2-oxopiperidine-3-carboxylate (0.110 g, 245 μmol, 95% yield) as a yellow solid. LCMS [M+Na]⁺=450.3 m/z.

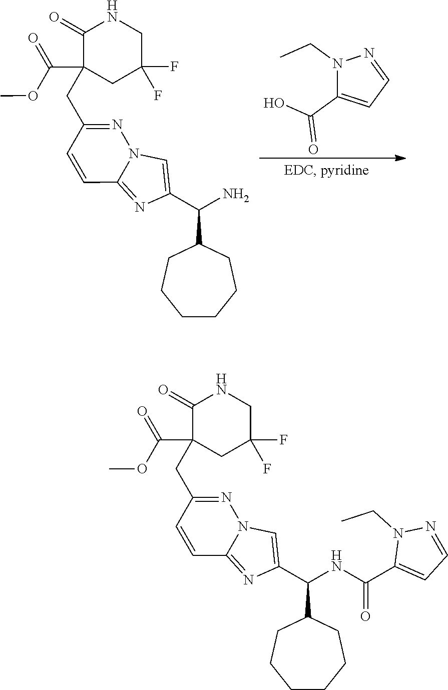

To a solution of methyl 3-((2-((S)-amino(cycloheptyl)methyl)imidazo[1,2-b]pyridazin-6-yl)methyl)-5,5-difluoro-2-oxopiperidine-3-carboxylate (0.110 g, 245 μmol, 1.00 eq) in pyridine (3.00 mL) was added EDCI (141 mg, 734 μmol, 3.00 eq) and 1-ethyl-1H-pyrazole-5-carboxylic acid (51.4 mg, 367 μmol, 1.50 eq). The mixture was stirred at 15° C. for 1 h. The reaction mixture diluted with H₂O and extracted with EtOAc. The combined organic layers were dried over Na₂SO₄, filtered, and concentrated under reduced pressure. The residue was purified by prep-TLC (SiO₂, DCM:MeOH=10:1) to afford methyl 3-((2-((S)-cycloheptyl(1-ethyl-1H-pyrazole-5-carboxamido)methyl)imidazo[1,2-b]pyridazin-6-yl)methyl)-5,5-difluoro-2-oxopiperidine-3-carboxylate (0.110 g, 192 μmol, 79% yield) as a white solid. LCMS [M+H]⁺=572.4 m/z.

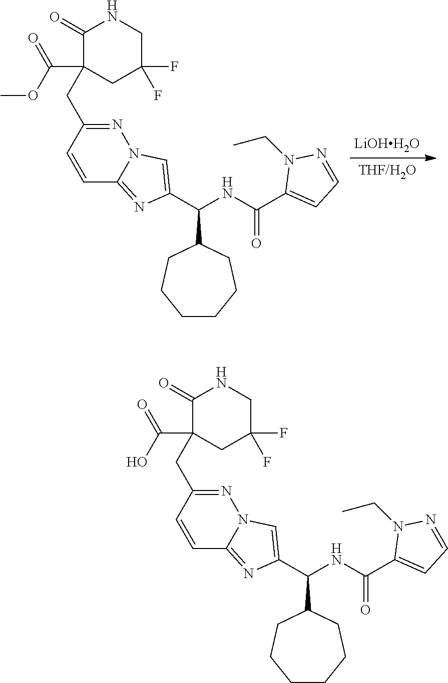

To a solution of methyl 3-((2-((S)-cycloheptyl(1-ethyl-1H-pyrazole-5-carboxamido) methyl)imidazo[1,2-b]pyridazin-6-yl)methyl)-5,5-difluoro-2-oxopiperidine-3-carboxylate (0.110 g, 192 μmol, 1.00 eq) in THE (3.00 mL) and H₂O (1.00 mL) was added LiOH·H₂O (24.2 mg 577 μmol, 3.00 eq). The mixture was stirred at 15° C. for 1 h. The reaction mixture pH was adjusted to 4 with 1 M HCl at 0° C. and concentrated under reduced pressure to give crude product, 3-((2-((S)-cycloheptyl(1-ethyl-1H-pyrazole-5-carboxamido)methyl)imidazo[1,2-b]pyridazin-6-yl)methyl)-5,5-difluoro-2-oxopiperidine-3-carboxylic acid, which was used directly in the next step.

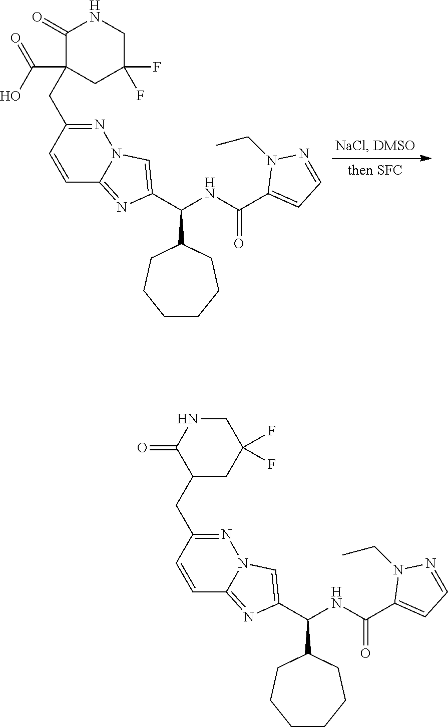 [SMILES: CCn1nccc1C(=O)N[C@H](c1cn2nc(CC3(C(=O)O)CC(F)(F)CNC3=O)ccc2n1)C1CCCCCC1 ; CCn1nccc1C(=O)N[C@H](c1cn2nc(CC3CC(F)(F)CNC3=O)ccc2n1)C1CCCCCC1]

To a solution of crude 3-((2-((S)-cycloheptyl(1-ethyl-1H-pyrazole-5-carboxamido)methyl)imidazo[1,2-b]pyridazin-6-yl)methyl)-5,5-difluoro-2-oxopiperidine-3-carboxylic acid from the previous step in DMSO (3.00 mL) was added NaCl (31.4 mg, 538 μmol, 3.00 eq). The mixture was stirred at 100° C. for 1 h. The reaction mixture was diluted with H₂O and extracted with EtOAc. The combined organic layers were washed with H₂O, dried over Na₂SO₄, filtered, and concentrated under reduced pressure to afford a mixture of diastereomers of N-((1 S)-cycloheptyl(6-((5,5-difluoro-2-oxopiperidin-3-yl)methyl)imidazo[1,2-b]pyridazin-2-yl)methyl)-1-ethyl-1H-pyrazole-5-carboxamide (0.045 g, 84.9 μmol, 44% yield over 2 steps) as a white solid. The residue was purified by SFC (column: DAICEL CHIRALPAK AS (250 mm*30 mm, 10 μm); mobile phase: [0.1% NH₃/H₂O MeOH]; B %: 35%-35%, 4.8 min, Rt=1.468, 1.614 min) to afford the separated title compounds as single isomers.

Compound 9: The title compound was obtained from SFC purification as the first eluting single stereoisomer (15.0 mg, 29.2 μmol, 38% yield) as a yellow solid. LCMS [M+H]⁺=514.4m/z.

Compound 10: The title compound was obtained from SFC purification as the second eluting single stereoisomer (15.0 mg, 29.2 μmol, 38% yield) as a yellow solid. LCMS [M+H]⁺=514.4m/z.

### Example 12: Preparation of Compounds 11 and 12

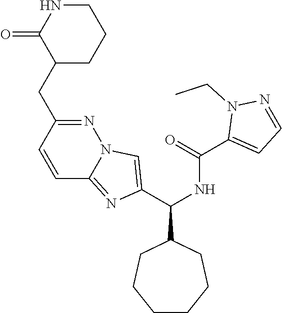 [SMILES: CCn1nccc1C(=O)N[C@H](c1cn2nc(CC3CCCNC3=O)ccc2n1)C1CCCCCC1]

### N-((1S)-cycloheptyl(6-((2-oxopiperidin-3-yl)methyl)imidazo[1,2-b]pyridazin-2-yl)methyl)-1-ethyl-1H-pyrazole-5-carboxamide. (Compounds 11 and 12)

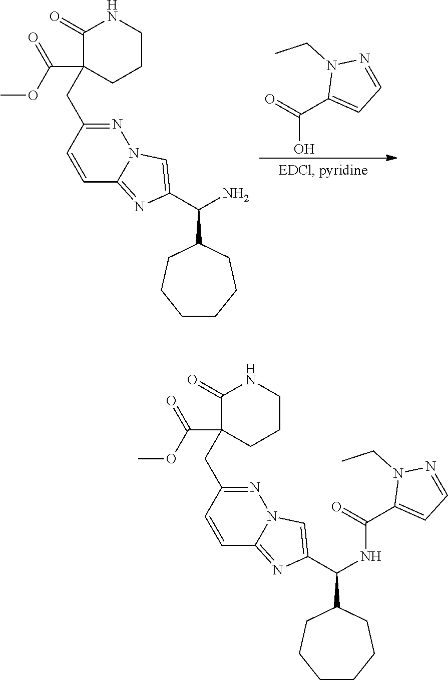

To a solution of methyl 3-((2-((S)-amino(cycloheptyl)methyl)imidazo[1,2-b]pyridazin-6-yl)methyl)-2-oxopiperidine-3-carboxylate (120 mg, 290 mmol, 1.00 eq) and 1-ethyl-1H-pyrazole-5-carboxylic acid (48.8 mg, 348 mmol, 1.20 eq) in pyridine (5.00 mL) was added EDCI (166 mg 870 mmol, 3.00 eq). The reaction mixture was stirred at RT for 1 h. The reaction mixture was diluted with water and extracted with EtOAc. The combined organic layers were washed with a saturated aqueous solution of NaCO₃, dried over anhydrous Na₂SO₄, filtered, and concentrated under reduced pressure. The resulting residue was purified by prep-TLC (SiO₂, DCM/MeOH) to afford methyl 3-((2-((S)-cycloheptyl(1-ethyl-1H-pyrazole-5-carboxamido)methyl)imidazo[1,2-b]pyridazin-6-yl)methyl)-2-oxopiperidine-3-carboxylate (100 mg, 186 mmol, 64% yield) as a white solid. LCMS [M+H]⁺=536.2 m/z.

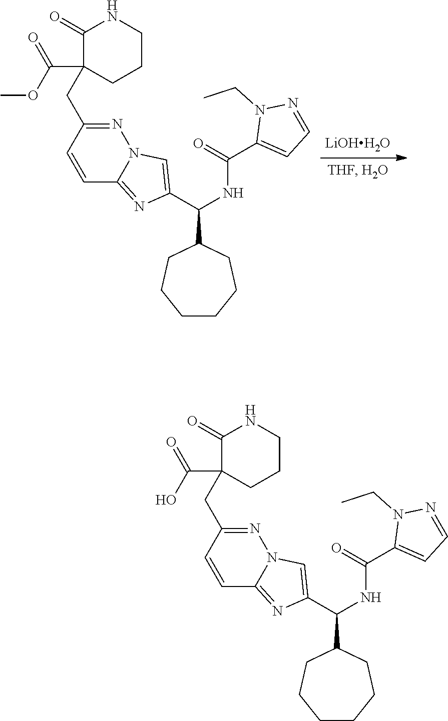

To a solution of methyl 3-((2-((S)-cycloheptyl(1-ethyl-1H-pyrazole-5-carboxamido)methyl)imidazo[1,2-b]pyridazin-6-yl)methyl)-2-oxopiperidine-3-carboxylate (100 mg, 186 mmol, 1.00 eq) in THF (3.00 mL) was added LiOH·H₂O (47.0 mg, 1.12 mmol, 6.00 eq) in water (1.00 mL). The reaction mixture was stirred at RT for 1 h. The reaction mixture was acidified to pH=2 by adding a 1 M aqueous solution of HCl. The reaction mixture was extracted with DCM. The combined organic layers were dried over anhydrous Na₂SO₄, filtered, and concentrated under reduced pressure to afford 3-((2-((S)-cycloheptyl(1-ethyl-1H-pyrazole-5-carboxamido)methyl)imidazo[1,2-b]pyridazin-6-yl)methyl)-2-oxopiperidine-3-carboxylic acid (90.0 mg, 172 mmol, 92% yield) as a white solid. LCMS [M+H]⁺=522.4 m/z.

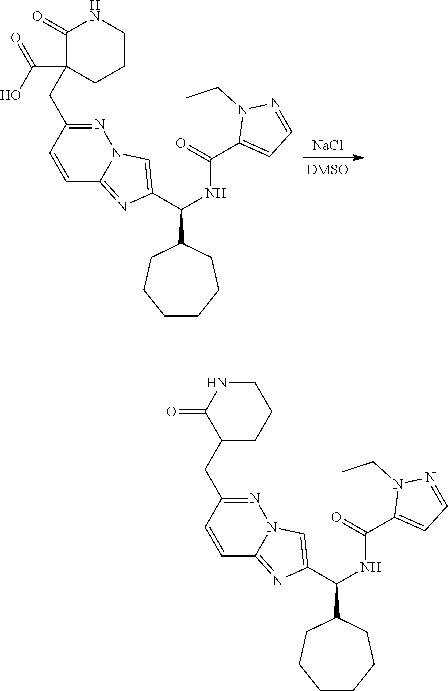

To a solution of 3-((2-((S)-cycloheptyl(1-ethyl-1H-pyrazole-5-carboxamido)methyl) imidazo[1,2-b]pyridazin-6-yl)methyl)-2-oxopiperidine-3-carboxylic acid (90.0 mg, 172 mmol, 1.00 eq) in DMSO (5.00 mL) was added NaCl (50.4 mg, 862 mmol, 5.00 eq). The reaction mixture was stirred at 80° C. for 1 h. The reaction mixture was diluted with water and extracted with EtOAc. The combined organic layers were washed with a saturated aqueous solution of NaCO₃, dried over anhydrous Na₂SO₄, filtered, and concentrated under reduced pressure. The resulting residue was purified by prep-TLC (SiO₂, DCM/MeOH) to afford N-((1S)-cycloheptyl(6-((2-oxopiperidin-3-yl)methyl)imidazo[1,2-b]pyridazin-2-yl)methyl)-1-ethyl-1H-pyrazole-5-carboxamide (59.2 mg, 122 mmol, 71% yield) as a white solid. LCMS [M+H]⁺=478.4 m/z.

Compound 11: The title compound was isolated as the first eluting, single stereoisomer by chiral SFC purification (column: Daicel Chiralpak AD (250 mm×30 mm, 10 mm); mobile phase: [0.1% NH₃H₂O in EtOH]; B %: 40%-40%, 3.16 min); (23.0 mg, 47.3 mmol, 40% yield) and was obtained as a white solid. LCMS [M+H]⁺=478.4 m/z.

Compound 12: The title compound was isolated as the second eluting, single stereoisomer by chiral SFC purification (column: Daicel Chiralpak AD (250 mm×30 mm, 10 mm); mobile phase: [0.10% NH₃H₂O in EtOH]; B %: 40%-40%, 3.16 min); (22.3 mg, 46.1 mmol, 399% yield) and was obtained as a white solid. LCMS [M+H]⁺=478.4 m/z.

### Example 13: Preparation of Compound 13

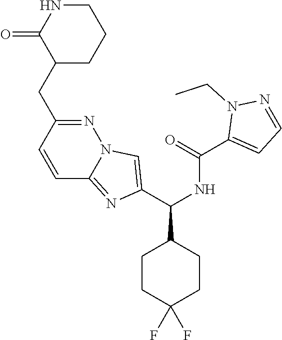 [SMILES: CCn1nccc1C(=O)N[C@H](c1cn2nc(CC3CCCNC3=O)ccc2n1)C1CCC(F)(F)CC1]

### N-((1 S)-(4,4-difluorocyclohexyl)(6-((2-oxopiperidin-3-yl)methyl)imidazo[1,2-b]pyridazin-2-yl)methyl)-1-ethyl-1H-pyrazole-5-carboxamide. (Compound 13)

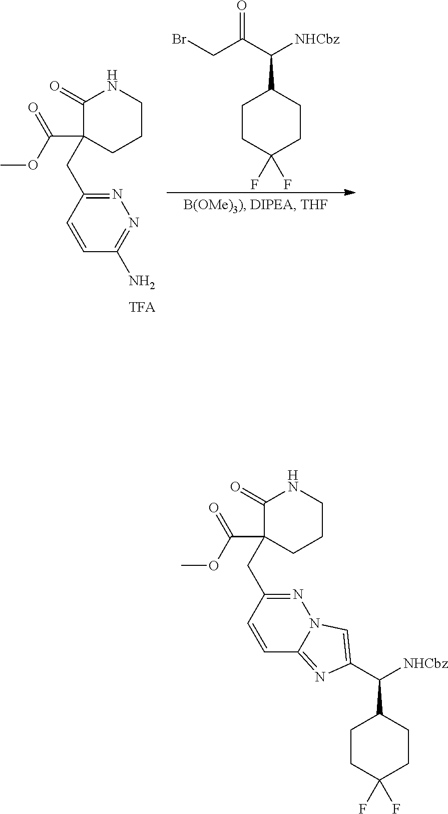 [SMILES: COC(=O)C1(Cc2ccc(N)nn2)CCCNC1=O ; O=C(N[C@H](C(=O)CBr)C1CCC(F)(F)CC1)OCc1ccccc1 ; COC(=O)C1(Cc2ccc3nc([C@@H](NC(=O)OCc4ccccc4)C4CCC(F)(F)CC4)cn3n2)CCCNC1=O]

To a solution of methyl 3-((6-aminopyridazin-3-yl)methyl)-2-oxopiperidine-3-carboxylate (350 mg, 925 μmol, 1.00 eq, TFA salt) and benzyl (S)-(3-bromo-1-(4,4-difluorocyclohexyl)-2-oxopropyl)carbamate (Int. 2; 374 mg, 925 μmol, 1.00 eq) in THF (4.00 mL) was added B(OMe)₃ (480 mg, 4.63 mmol, 522 μL, 5.00 eq) and DIPEA (1.20 g, 9.25 mmol, 1.61 mL, 10.0 eq). The mixture was stirred at 70° C. for 2 h. The residue was diluted with H₂O and extracted with EtOAc. The combined organic layers were dried over Na₂SO₄, filtered, and concentrated under reduced pressure. The residue was purified by prep-HPLC (column: Phenomenex Luna (C18 150*40 mm*15 μm); mobile phase: [H₂O (FA)-ACN]; B %: 40%-70%, 10 min) to afford methyl 3-((2-((S)-(((benzyloxy)carbonyl)amino)(4,4-difluorocyclohexyl)methyl)imidazo[1,2-b]pyridazin-6-yl)methyl)-2-oxopiperidine-3-carboxylate (220 mg, 386 μmol, 42% yield) as a yellow oil. LCMS [M+H]⁺=570.3 m/z.

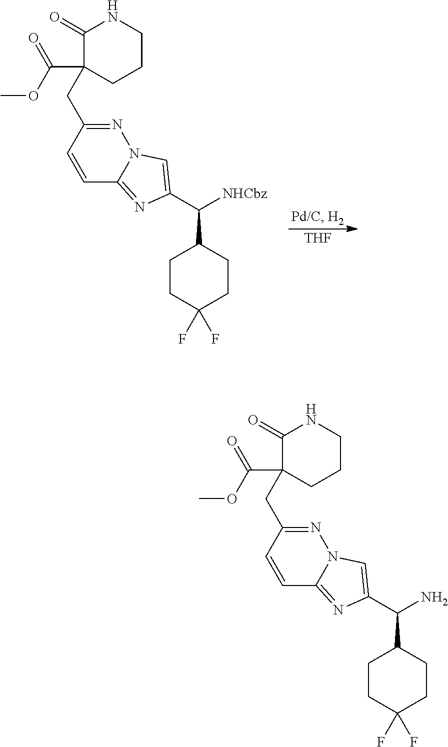 [SMILES: COC(=O)C1(Cc2ccc3nc([C@@H](NC(=O)OCc4ccccc4)C4CCC(F)(F)CC4)cn3n2)CCCNC1=O ; "Pd/C, H2" ; COC(=O)C1(Cc2ccc3nc([C@@H](N)C4CCC(F)(F)CC4)cn3n2)CCCNC1=O ; "THF"]

To a solution of methyl 3-((2-((S)-(((benzyloxy)carbonyl)amino)(4,4-difluorocyclohexyl)methyl)imidazo[1,2-b]pyridazin-6-yl)methyl)-2-oxopiperidine-3-carboxylate (200 mg, 351 μmol, 1.00 eq) in THF (4.00 mL) was added Pd/C (80.0 mg, 10% purity) under N₂. The suspension was degassed under vacuum and purged with H₂ three times. The mixture was stirred under H₂ (15.0 psi) at 18° C. for 20 h. The reaction mixture was filtered with MeOH washes, and the filtrate was concentrated under reduced pressure to afford methyl 3-((2-((S)-amino(4,4-difluorocyclohexyl)methyl)imidazo[1,2-b]pyridazin-6-yl)methyl)-2-oxopiperidine-3-carboxylate (120 mg, 275 μmol, 78% yield) as a yellow solid. LCMS [M+H]⁺=436.2 m/z.

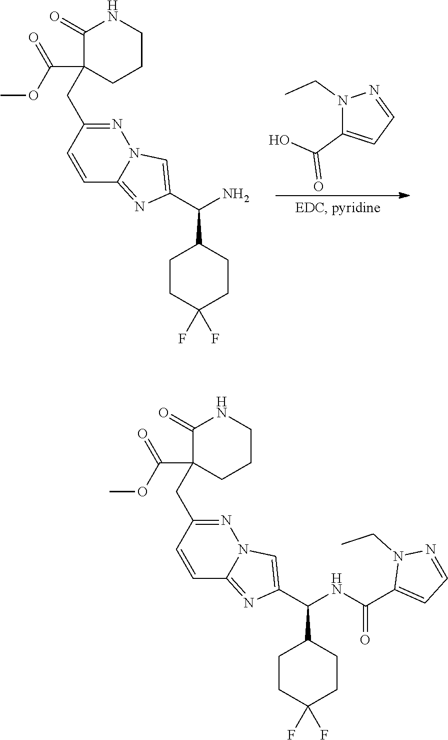

To a solution of methyl 3-((2-((S)-amino(4,4-difluorocyclohexyl)methyl)imidazo[1,2-b]pyridazin-6-yl)methyl)-2-oxopiperidine-3-carboxylate (120 mg, 275 μmol, 1.00 eq) and 1-ethyl-1H-pyrazole-5-carboxylic acid (57.9 mg, 413 μmol, 1.50 eq) in pyridine (2.00 mL) was added EDCI (264 mg, 1.38 mmol, 5.00 eq). The mixture was stirred at 18° C. for 2 h. The reaction mixture was then diluted with H₂O and extracted with EtOAc. The combined organic layers were dried over Na₂SO₄, filtered, and concentrated under reduced pressure. The residue was purified by prep-HPLC (column: Phenomenex Luna (C18 150*25 mm*10 μm); mobile phase: [water (FA)-ACN]; B %: 29%-59%, 10 min) to afford methyl 3-((2-((S)-(4,4-difluorocyclohexyl)(1-ethyl-1H-pyrazole-5-carboxamido)methyl)imidazo[1,2-b]pyridazin-6-yl)methyl)-2-oxopiperidine-3-carboxylate (100 mg, 179 μmol, 65% yield) as a yellow solid. LCMS [M+H]⁺=558.3 m/z.

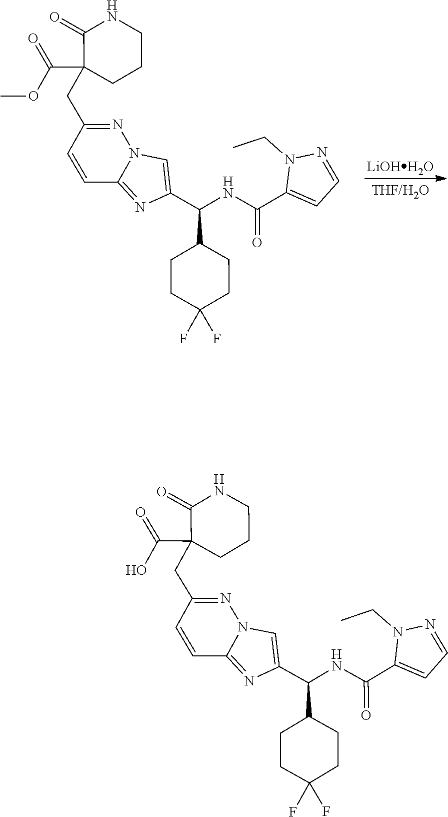

To a solution of methyl 3-((2-((S)-(4,4-difluorocyclohexyl)(1-ethyl-1H-pyrazole-5-carboxamido)methyl)imidazo[1,2-b]pyridazin-6-yl)methyl)-2-oxopiperidine-3-carboxylate (100 mg, 179 μmol, 1.00 eq) in THF (3.00 mL) was added LiOH·H₂O (22.5 mg, 538 μmol, 3.00 eq) and H₂O (0.500 mL). The mixture was stirred at 18° C. for 6 h. The reaction mixture pH was adjusted to 4 with 1 M HCl and then concentrated under reduced pressure to afford crude 3-((2-((S)-(4,4-difluorocyclohexyl)(1-ethyl-1H-pyrazole-5-carboxamido)methyl)imidazo[1,2-b]pyridazin-6-yl)methyl)-2-oxopiperidine-3-carboxylic acid, which was used in the next step without further purification. LCMS [M+H]⁺=544.3 m/z.

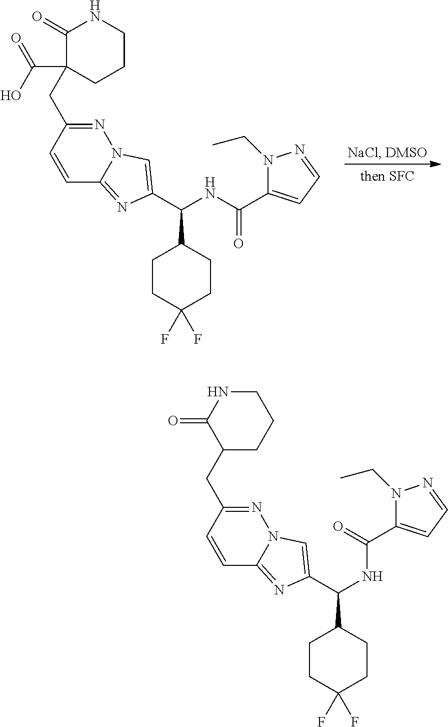

To a solution of crude 3-((2-((S)-(4,4-difluorocyclohexyl)(1-ethyl-1H-pyrazole-5-carboxamido)methyl)imidazo[1,2-b]pyridazin-6-yl)methyl)-2-oxopiperidine-3-carboxylic acid in DMSO (1.50 mL) was added NaCl (29.0 mg, 496 μmol, 3.00 eq). The mixture was stirred at 90° C. for 2 h. The reaction mixture was diluted with H₂O and extracted with EtOAc. The combined organic layers were dried over Na₂SO₄, filtered, and concentrated under reduced pressure. The residue was purified by prep-HPLC (column: Phenomenex Luna (C18 150*25 mm*10 μm); mobile phase: [water (FA)-ACN]; B %: 26%-56%, 10 min) to afford a mixture of diastereomers of N-((1 S)-(4,4-difluorocyclohexyl)(6-((2-oxopiperidin-3-yl)methyl)imidazo[1,2-b]pyridazin-2-yl)methyl)-1-ethyl-1H-pyrazole-5-carboxamide as a white solid. The mixture of diastereomers was then purified by SFC (column: DAICEL CHIRALPAK AD (250 mm*30 mm, 10 μm); mobile phase: [0.1% NH₃/H₂O EtOH]; B %: 40%-40%, 3.6 min, Rt=1.949 min) with the second eluting single stereoisomer as the title compound (24.28 mg, 46.6 μmol, 26% yield over 2 steps), obtained as a white solid. LCMS [M+H]⁺=500.3 m/z.

### Example 14: Preparation of Compound 14

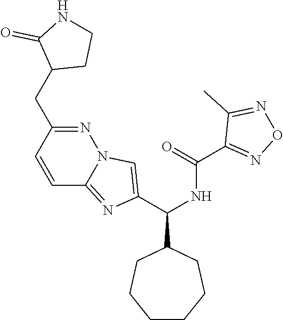 [SMILES: Cc1nonc1C(=O)N[C@H](c1cn2nc(CC3CCNC3=O)ccc2n1)C1CCCCCC1]

### N-((1 S)-cycloheptyl(6-((2-oxopyrrolidin-3-yl)methyl)imidazo[1,2-b]pyridazin-2-yl)methyl)-4-methyl-1,2,5-oxadiazole-3-carboxamide. (Compound 14)

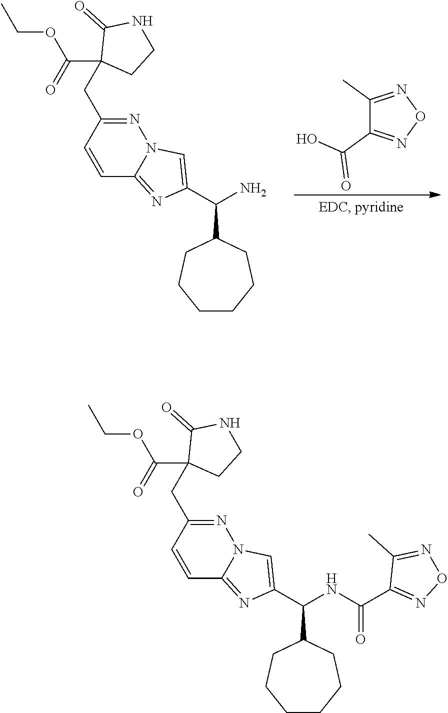

To a solution of ethyl 3-((2-((S)-amino(cycloheptyl)methyl)imidazo[1,2-b]pyridazin-6-yl)methyl)-2-oxopyrrolidine-3-carboxylate (130 mg, 314 μmol, 1.00 eq) and 4-methyl-1,2,5-oxadiazole-3-carboxylic acid (80.5 mg, 628 μmol, 2.00 eq) in pyridine (2.00 mL) was added EDCI (180 mg, 943 μmol, 3.00 eq). The mixture was stirred at RT for 1 h. The reaction mixture was diluted with H₂O and extracted with EtOAc. The combined organic layers were dried over anhydrous Na₂SO₄, filtered, and concentrated under vacuum. The residue was purified by prep-TLC (SiO₂, DCM:MeOH=10:1) to afford ethyl 3-((2-((S)-cycloheptyl(4-methyl-1,2,5-oxadiazole-3-carboxamido)methyl)imidazo[1,2-b]pyridazin-6-yl)methyl)-2-oxopyrrolidine-3-carboxylate (130 mg, 248.29 μmol, 79% yield) as a brown oil. LCMS [M+H]⁺=524.4 m/z.

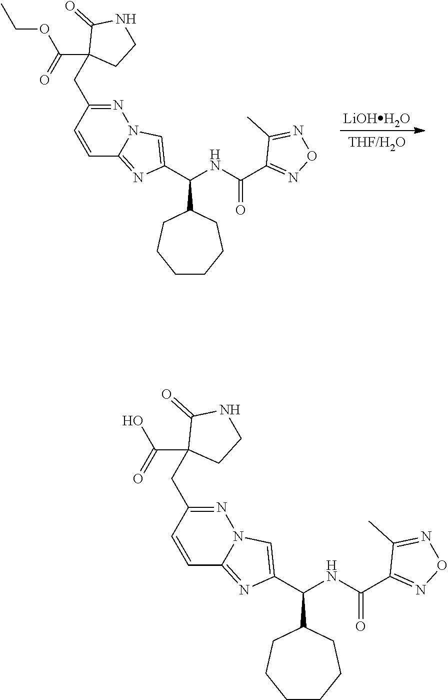

To a solution of ethyl 3-((2-((S)-cycloheptyl(4-methyl-1,2,5-oxadiazole-3-carboxamido)methyl)imidazo[1,2-b]pyridazin-6-yl)methyl)-2-oxopyrrolidine-3-carboxylate (130 mg, 248 μmol, 1.00 eq) in THF (4.00 mL) and H₂O (2.00 mL) was added LiOH·H₂O (20.8 mg 496 μmol, 2.00 eq) at 0° C. The mixture was stirred at RT for 1 h. The reaction mixture was diluted with H₂O, adjusted to pH 5 with 1 M HCl, and extracted with EtOAc. The combined organic layers were dried over anhydrous Na₂SO₄, filtered, and concentrated under reduced pressure to give 3-((2-((S)-cycloheptyl(4-methyl-1,2,5-oxadiazole-3-carboxamido)methyl)imidazo[1,2-b]pyridazin-6-yl)methyl)-2-oxopyrrolidine-3-carboxylic acid (80.0 mg, 161 μmol, 65% yield) as a yellow solid. LCMS [M+H]⁺=496.3 m/z.

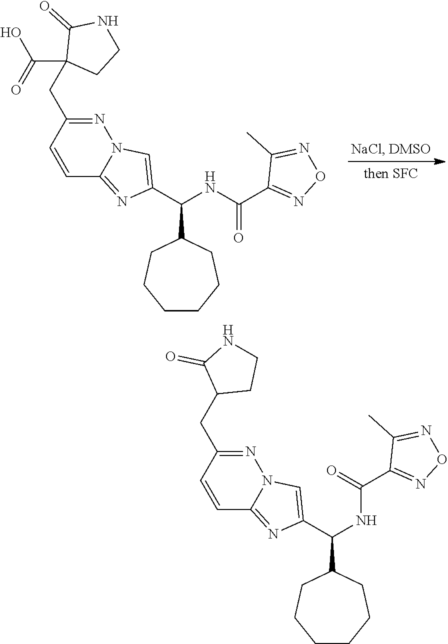

To a solution of 3-((2-((S)-cycloheptyl(4-methyl-1,2,5-oxadiazole-3-carboxamido)methyl) imidazo[1,2-b]pyridazin-6-yl)methyl)-2-oxopyrrolidine-3-carboxylic acid (80.0 mg, 161 μmol, 1.00 eq) in DMSO (2.00 mL) was added NaCl (37.7 mg, 645 μmol, 4.00 eq). The mixture was stirred at 25° C. for 2 h. The reaction mixture was diluted with H₂O and extracted with EtOAc. The combined organic layers were dried with anhydrous Na₂SO₄, filtered, and concentrated under vacuum. The residue was purified by prep-TLC (SiO₂, DCM:MeOH=10:1) to give a mixture of diastereomers, N-((1S)-cycloheptyl(6-((2-oxopyrrolidin-3-yl)methyl)imidazo[1,2-b]pyridazin-2-yl)methyl)-4-methyl-1,2,5-oxadiazole-3-carboxamide (43.8 mg, 93.7 μmol, 58% yield), as white solid. The mixture of diastereomers was then purified by SFC (column: DAICEL CHIRALPAK AS (250 mm*30 mm, 10 μm); mobile phase: [ACN/MeOH (0.1% NH₃/H₂O)]; B %: 45%-45%, 5.20 min, Rt=1.661 min). The first eluting single isomer (9.39 mg, 21.6 μmol, 22% yield) was obtained as white solid. LCMS [M+H]⁺=452.3 m/z.

### Example 15: General Procedure 1

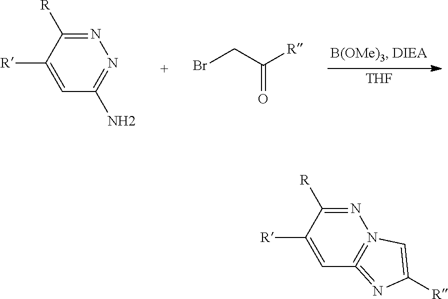

To a solution of aminopyridazine (1.00 eq) and bromoketone (1.00-1.20 eq) in THF was added DIEA (5.00-10.0 eq) and trimethyl borate (5.00-10.0 eq). The reaction mixture was stirred at 70° C. for 1-3 h. The reaction mixture was then diluted with water and extracted with EtOAc. The combined organic layers were washed with water then brine, dried over Na₂SO₄, filtered, and concentrated under reduced pressure to give a residue, which was then purified by prep-TLC, column chromatography, prep-HPLC, and/or prep-SFC to afford imidazo[1,2-b]pyridazine.

### Example 16: General Procedure 2

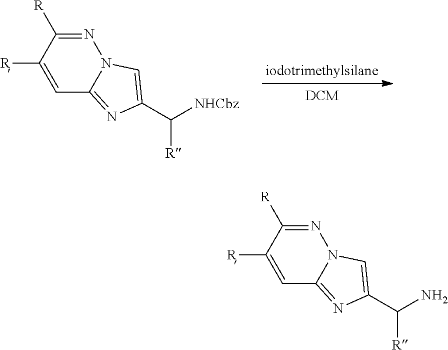

To a solution of Cbz-protected primary amine (1.00 eq) in DCM was added iodotrimethylsilane (1.50-8.55 eq) at 0° C. The reaction mixture was stirred at 0° C. for 2 h. The reaction mixture was diluted with 1 M HCl and washed with EtOAc. The aqueous phase was adjusted to pH=8-9 with saturated aqueous NaHCO₃ and extracted with EtOAc. The combined organic layers were dried over anhydrous Na₂SO₄, filtered, and concentrated under reduced pressure to afford free primary amine.

### Example 17: General Procedure 3

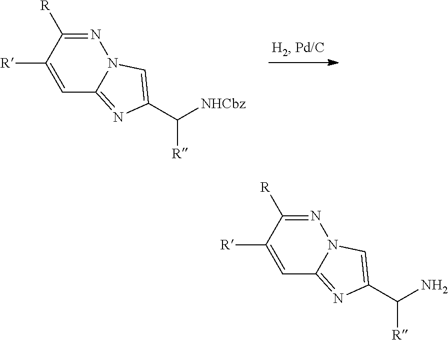

To a solution of Cbz-protected primary amine (1.00 eq) in THF, DCM, and/or MeOH (optionally with additive NH₃·H₂O), was added Pd/C (10.0% purity) under N₂ atmosphere. The suspension was degassed and purged with H₂ 3 times. The reaction mixture was stirred under H₂ (15 psi) at RT for 1-20 h. The reaction mixture was filtered then concentrated under reduced pressure to afford free primary amine.

### Example 18: General Procedure 4

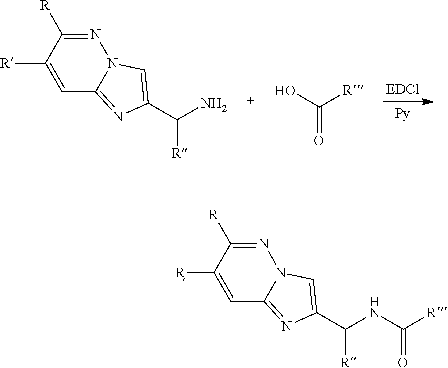

A solution of primary amine (1.00 eq), carboxylic acid (1.20-3.00 eq), and EDCI (2.00-3.00 eq) in pyridine were stirred at RT for 1-18 h. The reaction mixture was then diluted with water and extracted with EtOAc. The combined organic layers were optionally washed with water then brine, dried over Na₂SO₄, filtered, and concentrated under reduced pressure to give a residue, and the residue was purified by prep-TLC, column chromatography, prep-HPLC, and/or prep-SFC to afford amide.

### Example 19: General Procedure 5

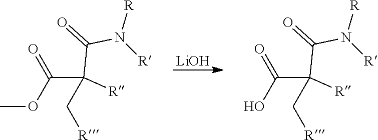

To a solution of ester in THF and H₂O was added LiOH·H₂O. The reaction mixture was stirred at RT for 0.5-48 h. The reaction mixture was optionally diluted with water and washed with EtOAc. The aqueous layer was adjusted to pH=2-7 with HCl (1 M) and extracted with EtOAc or DCM. The combined organic layers were dried over Na₂SO₄, filtered, and concentrated under reduced pressure to give carboxylic acid.

### Example 20: General Procedure 6

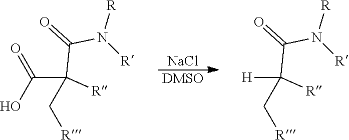

To a solution of carboxylic acid in DMF or DMSO was added NaCl (excess). The reaction mixture was stirred at 100-130° C. for 0.5-10 h. The reaction mixture was allowed to cool, then optionally diluted with H₂O and extracted with EtOAc, DCM, or DCM/MeOH. The combined organic layers were dried over Na₂SO₄, filtered, and concentrated to give a residue. The residue was purified by prep-TLC, column chromatography, prep-HPLC, and/or prep-SFC to afford decarboxylated product.

### Example 21: General Procedure 7

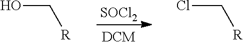

To a solution of benzylic alcohol (1.00 eq) in DCM (0.25 M) was added SOCl₂ (3.00 eq) at 0° C. The reaction mixture was stirred for 1 h before concentrating under reduced pressure at 30° C. to afford crude benzylic chloride as the desired product, which was used directly in the next step.

### Example 22: General Procedure 8

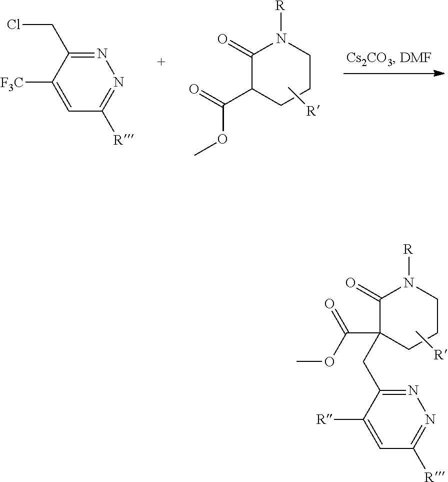

To a solution of the requisite 2-oxopiperidine-3-carboxylate ester (1.2 eq) in DMF (0.65 M) was added Cs₂CO₃ (2.0 eq). The reaction mixture was stirred for 30 min before adding benzylic chloride (1.0 eq). The reaction mixture was stirred at RT for 12 h. The reaction mixture was diluted with water (60 mL) and extracted with EtOAc (30 mL×3). The combined organic layers were washed with brine, dried over Na₂SO₄, filtered, and concentrated under reduced pressure to afford a residue, which was purified by prep-HPLC (FA in ACN/water) to afford the desired product.

### Example 23: General Procedure 9

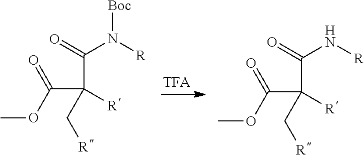

To a solution of Boc-protected starting material (1.0 eq) in DCM (0.1 M) was added TFA (˜100 eq) at 0° C. The reaction mixture was stirred at RT until the starting material (SM) was consumed (monitored by LCMS). The reaction mixture was concentrated under reduced pressure to afford the desired compound as a TFA salt, which was used directly in the next reaction without further purification.

### Example 24: General Procedure 10

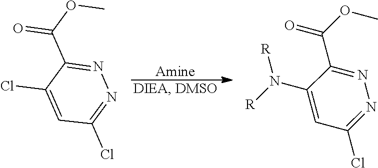

To a solution of methyl 4,6-dichloropyridazine-3-carboxylate (1.00 eq) in DMSO (0.5 M) was added amine (1.20 eq) and DIEA (2.00 eq). The reaction mixture was stirred at 80° C. for 1 h. The residue was diluted with H₂O and extracted with DCM. The combined organic layers were dried over Na₂SO₄, filtered, and concentrated under reduced pressure to give a residue. The residue was purified by column chromatography (1:1 PE:EtOAc) to afford the S_{N}Ar product.

### Example 25: General Procedure 11

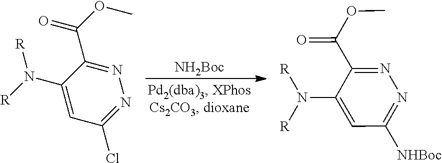

A mixture of chloropyridazine (1.00 eq), tert-butyl carbamate (5.00 eq), K₂CO₃ (3.00 eq), XPhos (0.100 eq), and Pd₂(dba)₃ (0.100 eq) in toluene (0.2 M) was degassed and purged with N₂ and stirred at 80° C. for 2 h. The reaction mixture was diluted with H₂O and extracted with DCM The combined organic layers were dried over Na₂SO₄, filtered, and concentrated under reduced pressure to give a residue. The residue was purified by column chromatography or prep-HPLC to afford the substituted tert-butyl pyridazin-3-ylcarbamate.

### Example 26: General Procedure 12

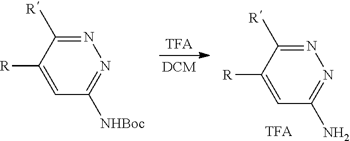

To a solution of substituted tert-butyl pyridazin-3-ylcarbamate (1.00 eq) in DCM (0.5-1 M) was added TFA (˜10 eq). The reaction mixture was stirred for 2 h before concentrating under reduced pressure to afford the aminopyridazine as a TFA salt, which was used directly in the next reaction.

### Example 27: General Procedure 13

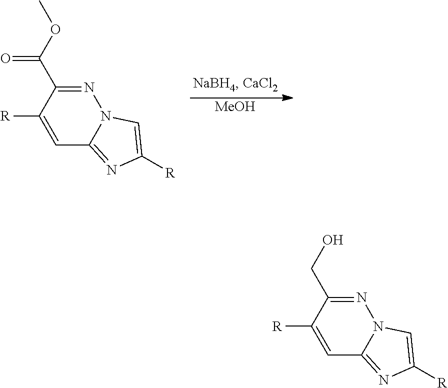

To a solution of ester (1.0 eq) in MeOH (0.25 M) was added CaCl₂ (2.0 eq) and NaBH₄ (5.0 eq) at 0° C. The reaction mixture was stirred at RT for 2 h before treating with saturated aqueous NH₄Cl at 0° C. and diluting with water. The reaction mixture was extracted with EtOAc, washed with brine, dried over anhydrous Na₂SO₄, filtered, and concentrated under reduced pressure to afford the alcohol, which was used in the next step without further purification.

### Example 29: General Procedure 14

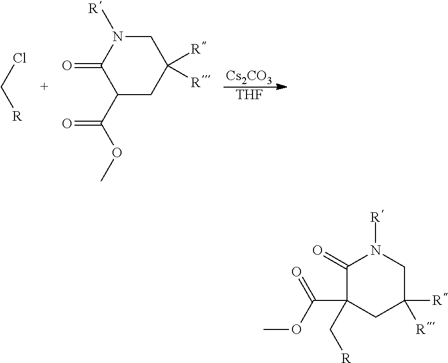

To a solution of chloride (1.0 eq) in THE (0.15 M) was added Cs₂CO₃ (3.0 eq) and substituted methyl 2-oxopiperidine-3-carboxylate (2.0 eq). The reaction mixture was stirred at 70° C. for 2 h before diluting with water and extracting with EtOAc. The organic layers were dried over anhydrous Na₂SO₄, filtered, and concentrated under reduced pressure to afford the requisite alkylation product, which was used in the next step without further purification.

### Example 30: General Procedure 15

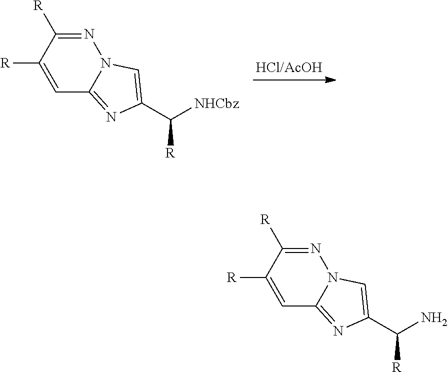

A solution of benzyl carbamate (1.00 eq) in HCl (0.2 M) and AcOH (0.02 M) was stirred at 50° C. for 30 min. The reaction mixture was adjusted with NaHCO₃ to pH ˜8, and then extracted with DCM. The combined organic layers were dried over anhydrous Na₂SO₄, filtered, and concentrated under reduced pressure to afford the requisite amine, which was used directly in the next reaction without further purification.

### Example 31: Preparation of Compound 15

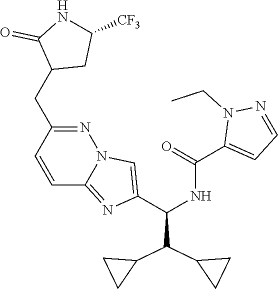 [SMILES: CCn1nccc1C(=O)N[C@H](c1cn2nc(CC3C[C@@H](C(F)(F)F)NC3=O)ccc2n1)C(C1CC1)C1CC1]

Compound 15 was isolated by chiral prep-SFC (DAICEL CHIRALCEL OJ-H (250 mm×30 mm, 5 um); 20% mobile phase: [0.1% NH₃H₂O EtOH]) as the first eluting stereoisomer of N-((1S)-2,2-dicyclopropyl-1-(6-(((5S)-2-oxo-5-(trifluoromethyl)pyrrolidin-3-yl)methyl)imidazo[1,2-b]pyridazin-2-yl)ethyl)-1-ethyl-1H-pyrazole-5-carboxamide (19.0 mg, 35.2 μmol, 22% yield), obtained as a white solid. LCMS [M+H]⁺=530.4 m/z.

### Example 32: Preparation of Compound 16

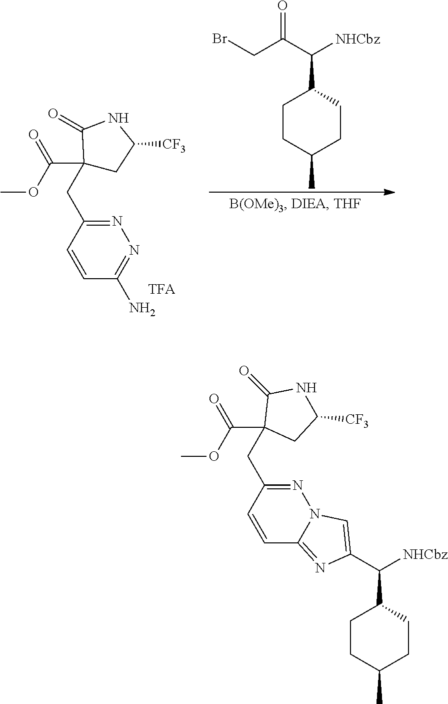 [SMILES: COC(=O)C1(Cc2ccc(N)nn2)C[C@@H](C(F)(F)F)NC1=O ; C[C@H]1CC[C@H]([C@H](NC(=O)OCc2ccccc2)C(=O)CBr)CC1 ; COC(=O)C1(Cc2ccc3nc([C@@H](NC(=O)OCc4ccccc4)[C@H]4CC[C@H](C)CC4)cn3n2)C[C@@H](C(F)(F)F)NC1=O]

A mixture of methyl (5S)-3-((6-aminopyridazin-3-yl)methyl)-2-oxo-5-(trifluoromethyl)pyrrolidine-3-carboxylate (160 mg, 370 μmol, 1.00 eq, TFA salt), benzyl ((S)-3-bromo-1-((1r,4S)-4-methylcyclohexyl)-2-oxopropyl)carbamate (212 mg, 555 μmol, 1.50 eq), DIEA (287 mg, 2.22 mmol, 386 μL, 6.00 eq), then B(OMe)₃ (230 mg, 2.22 mmol, 250 μL, 6.00 eq) in THF (4.00 mL) was degassed and purged with N₂ 3 times. Next, the reaction mixture was stirred at 70° C. for 3 h under N₂ atmosphere. The reaction mixture was diluted with H₂O (10.0 mL) and extracted with EtOAc (90.0 mL), dried over Na₂SO₄, filtered, and concentrated under reduced pressure to give a residue. The residue was purified by prep-TLC (DCM:MeOH=10:1). Methyl (5S)-3-((2-((S)-(((benzyloxy)carbonyl)amino)((1r,4S)-4-methylcyclohexyl)methyl)imidazo[1,2-b]pyridazin-6-yl)methyl)-2-oxo-5-(trifluoromethyl)pyrrolidine-3-carboxylate (200 mg, 332 μmol, 90% yield) was obtained as a white solid. LCMS [M+H]⁺=602.4 m/z.

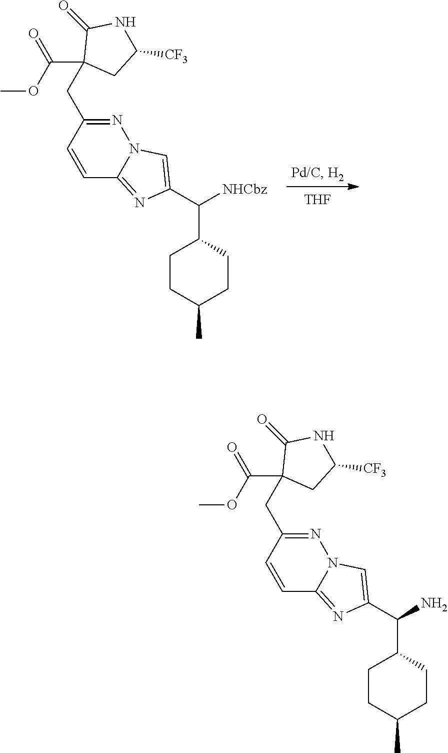 [SMILES: COC(=O)C1(Cc2ccc3nc(C(NC(=O)OCc4ccccc4)[C@H]4CC[C@H](C)CC4)cn3n2)C[C@@H](C(F)(F)F)NC1=O ; "Pd/C, H2" ; "THF" ; COC(=O)C1(Cc2ccc3nc([C@@H](N)[C@H]4CC[C@H](C)CC4)cn3n2)C[C@@H](C(F)(F)F)NC1=O]

To a solution of methyl (5S)-3-((2-((S)-(((benzyloxy)carbonyl)amino)((1r,4S)-4-methylcyclohexyl)methyl)imidazo[1,2-b]pyridazin-6-yl)methyl)-2-oxo-5-(trifluoromethyl)pyrrolidine-3-carboxylate (200 mg, 332 μmol, 1.00 eq) in THF (5.00 mL) was added Pd/C (100 mg, 10% purity). The reaction mixture was stirred at RT for 1 h under H₂ (15 psi). The reaction mixture was filtered and concentrated under reduced pressure to give a residue. Methyl (5S)-3-((2-((S)-amino((1r,4S)-4-methylcyclohexyl)methyl)imidazo[1,2-b]pyridazin-6-yl)methyl)-2-oxo-5-(trifluoromethyl)pyrrolidine-3-carboxylate (150 mg, 320 μmol, 97% yield) was obtained as a white solid. Crude product was used directly in the next step without further purification.

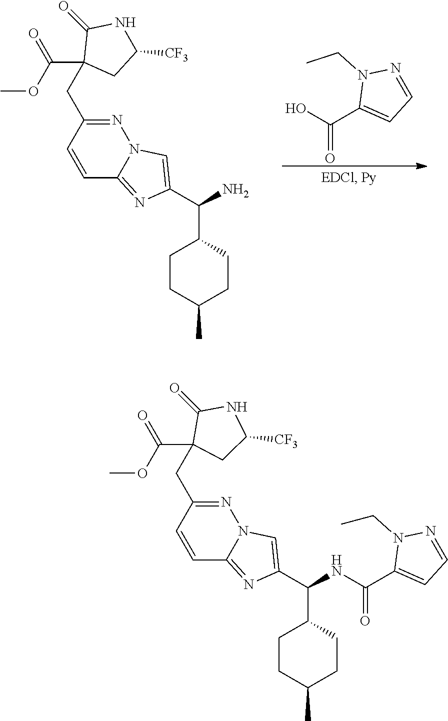

To a solution of methyl (5S)-3-((2-((S)-amino((1r,4S)-4-methylcyclohexyl)methyl)imidazo[1,2-b]pyridazin-6-yl)methyl)-2-oxo-5-(trifluoromethyl)pyrrolidine-3-carboxylate (150 mg, 320 μmol, 1.00 eq) and 1-ethyl-1H-pyrazole-5-carboxylic acid (67.4 mg, 481 μmol, 1.50 eq) in pyridine (3.00 mL) was added EDCI (246 mg 1.28 mmol, 4.00 eq). The reaction mixture was stirred at RT for 11 h. The reaction mixture was partitioned between EtOAc (60.0 mL) and H₂O (60.0 mL). The organic phase was separated, dried over Na₂SO₄, filtered, and concentrated under reduced pressure to give a residue. Methyl (5S)-3-((2-((S)-(1-ethyl-1H-pyrazole-5-carboxamido)((1r,4S)-4-methylcyclohexyl)methyl)imidazo[1,2-b]pyridazin-6-yl)methyl)-2-oxo-5-(trifluoromethyl)pyrrolidine-3-carboxylate (150 mg, 254 μmol, 79% yield) was obtained as a yellow oil. LCMS [M+H]⁺=590.4 m/z.

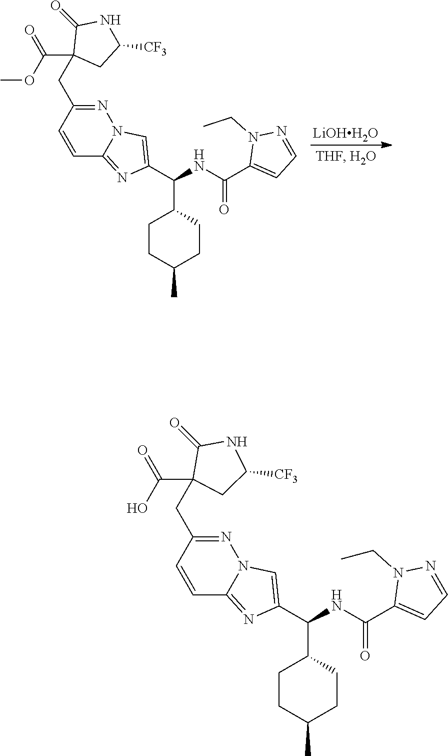

A solution of methyl (5S)-3-((2-((S)-(1-ethyl-1H-pyrazole-5-carboxamido)((1r,4S)-4-methylcyclohexyl)methyl)imidazo[1,2-b]pyridazin-6-yl)methyl)-2-oxo-5-(trifluoromethyl)pyrrolidine-3-carboxylate (150 mg, 254 μmol, 1.00 eq) in THF (8.00 mL) and H₂O (5.00 mL) was added LiOH·H₂O (21.3 mg, 508 μmol, 2.00 eq). The reaction mixture was stirred at RT for 2 h. The reaction mixture was adjusted pH to 5 with HCl (1 M), the reaction mixture was partitioned between EtOAc (60.0 mL) and H2O (60.0 mL). The organic phase was separated, dried over Na₂SO₄, filtered, and concentrated under reduced pressure to give a residue. (5S)-3-((2-((S)-(1-ethyl-1H-pyrazole-5-carboxamido)((1r,4S)-4-methylcyclohexyl)methyl)imidazo[1,2-b]pyridazin-6-yl)methyl)-2-oxo-5-(trifluoromethyl)pyrrolidine-3-carboxylic acid (120 mg, 208 μmol, 82% yield) was obtained as a yellow oil. LCMS [M+H]⁺=576.3 m/z.

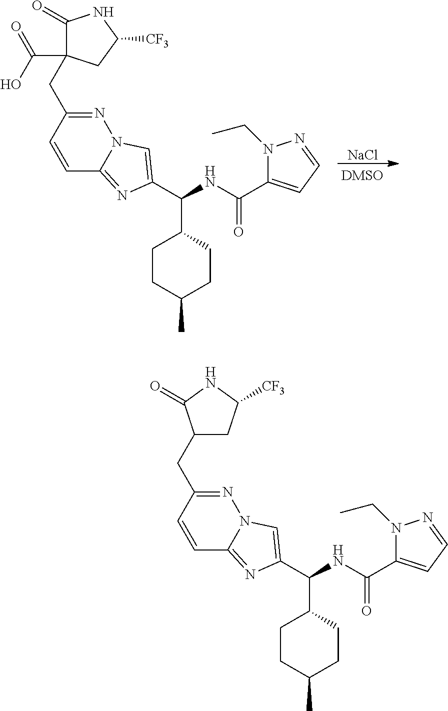

To a solution of (5S)-3-((2-((S)-(1-ethyl-1H-pyrazole-5-carboxamido)((1r,4S)-4-methylcyclohexyl)methyl)imidazo[1,2-b]pyridazin-6-yl)methyl)-2-oxo-5-(trifluoromethyl)pyrrolidine-3-carboxylic acid (120 mg, 208 μmol, 1.00 eq) in DMSO (2.00 mL) was added NaCl (60.9 mg, 1.04 mmol, 5.00 eq). The reaction mixture was stirred at 120° C. for 1 h. The reaction mixture was then filtered and concentrated to give a residue. The residue was purified by prep-HPLC (neutral condition; Waters Xbridge 150*25 mm*5 μm; mobile phase: [water (NH₄HCO₃)-ACN]; B %: 40%-70%, 8 min), the eluent was concentrated to remove organic solvents. The residual aqueous solution was lyophilized to give a white solid. Crude 1-ethyl-N-((1S)-((1r,4S)-4-methylcyclohexyl)(6-(((5S)-2-oxo-5-(trifluoromethyl)pyrrolidin-3-yl)methyl)imidazo[1,2-b]pyridazin-2-yl)methyl)-1H-pyrazole-5-carboxamide (104 mg, 195 μmol, 94% yield) was obtained as a mixture of stereoisomers. LCMS [M+H]⁺=532.4 m/z. Purification was then carried out using prep-SFC (DAICEL CHIRALCEL OJ (250 mm×30 mm, 10 μm); mobile phase: [0.1% NH₃H₂O EtOH]; B %: 15%). The eluent of the second eluting stereoisomer was concentrated to remove organic solvents. The residual aqueous solution was lyophilized to give a single stereoisomer of Compound 16 1-ethyl-N-((1S)-((1r,4S)-4-methylcyclohexyl)(6-(((5S)-2-oxo-5-(trifluoromethyl)pyrrolidin-3-yl)methyl)imidazo[1,2-b]pyridazin-2-yl)methyl)-1H-pyrazole-5-carboxamide (74.19 mg, 137 μmol, 70% yield), isolated as a white solid. LCMS [M+H]⁺=532.3 m/z.

### Example 33: Preparation of Compound 17

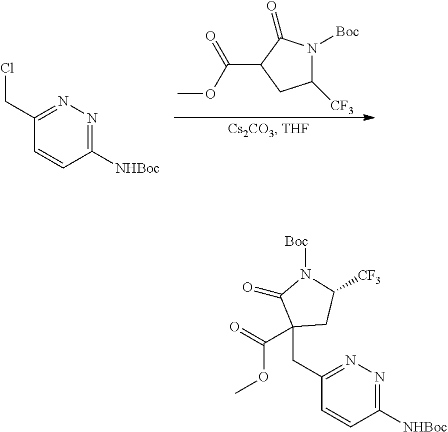 [SMILES: CC(C)(C)OC(=O)Nc1ccc(CCl)nn1 ; COC(=O)C1CC(C(F)(F)F)N(C(=O)OC(C)(C)C)C1=O ; COC(=O)C1(Cc2ccc(NC(=O)OC(C)(C)C)nn2)C[C@@H](C(F)(F)F)N(C(=O)OC(C)(C)C)C1=O]

A solution of tert-butyl (6-(chloromethyl)pyridazin-3-yl)carbamate (2.75 g, 8.85 mmol, 1.10 eq) and Cs₂CO₃ (5.24 g, 16.0 mmol, 2.00 eq) in THF (50.0 mL) was stirred at RT for 30 min. Then 1-(tert-butyl) 3-methyl (5S)-2-oxo-5-(trifluoromethyl)pyrrolidine-1,3-dicarboxylate (1.96 g 8.04 mmol, 1.00 eq) was added and stirred at 55° C. for 3 h. The residue was diluted with H₂O (60.0 mL) and extracted with EtOAc (60.0 mL×3), dried over Na₂SO₄, filtered, and concentrated under reduced pressure to give a residue. Crude product, 1-(tert-butyl) 3-methyl (5S)-3-((6-((tert-butoxy carbonyl)amino)pyridazin-3-yl)methyl)-2-oxo-5-(trifluoromethyl)pyrrolidine-1,3-dicarboxylate (5.40 g, crude), was obtained as a white oil. LCMS [M+H]⁺=519.6 m/z.

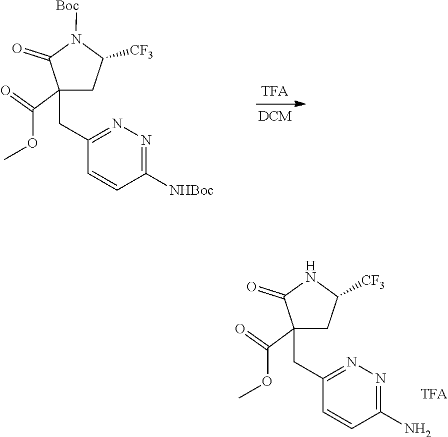 [SMILES: COC(=O)C1(Cc2ccc(NC(=O)OC(C)(C)C)nn2)C[C@@H](C(F)(F)F)N(C(=O)OC(C)(C)C)C1=O ; COC(=O)C1(Cc2ccc(N)nn2)C[C@@H](C(F)(F)F)NC1=O]

To a solution of 1-(tert-butyl) 3-methyl (5S)-3-((6-((tert-butoxycarbonyl)amino)pyridazin-3-yl)methyl)-2-oxo-5-(trifluoromethyl)pyrrolidine-1,3-dicarboxylate (5.30 g, 10.2 mmol, 1.00 eq) in DCM (60.0 mL) was added TFA (15.4 g, 135 mmol, 10.0 mL, 13.2 eq). The reaction mixture was stirred at 40° C. for 12 h. The reaction mixture was concentrated under the vacuum to give crude methyl (5S)-3-((6-aminopyridazin-3-yl)methyl)-2-oxo-5-(trifluoromethyl)pyrrolidine-3-carboxylate (5.60 g, TFA salt) as a red solid. LCMS [M+H]⁺=318.8 m/z.

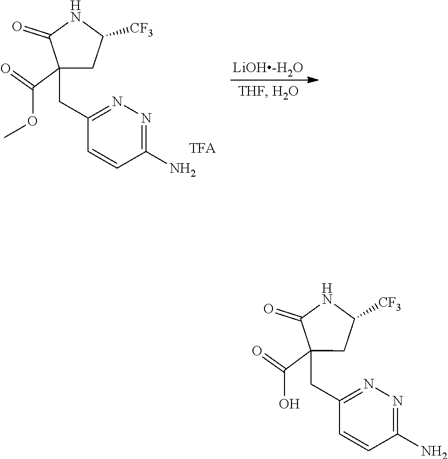 [SMILES: COC(=O)C1(Cc2ccc(N)nn2)C[C@@H](C(F)(F)F)NC1=O ; Nc1ccc(CC2(C(=O)O)C[C@@H](C(F)(F)F)NC2=O)nn1]

To a solution of crude methyl (5S)-3-((6-aminopyridazin-3-yl)methyl)-2-oxo-5-(trifluoromethyl)pyrrolidine-3-carboxylate (4.50 g, 10.4 mmol, 1.00 eq, TFA salt) in H₂O (13.0 mL) and THF (36.0 mL) was added LiOH·H₂O (1.75 g, 41.6 mmol, 4.00 eq). The reaction mixture was stirred at RT for 6 h. The reaction mixture was concentrated under the vacuum to give a residue. (5S)-3-((6-aminopyridazin-3-yl)methyl)-2-oxo-5-(trifluoromethyl)pyrrolidine-3-carboxylic acid (4.30 g, crude Li salt) was obtained as a yellow oil. LCMS [M+H]⁺=304.8 m/z.

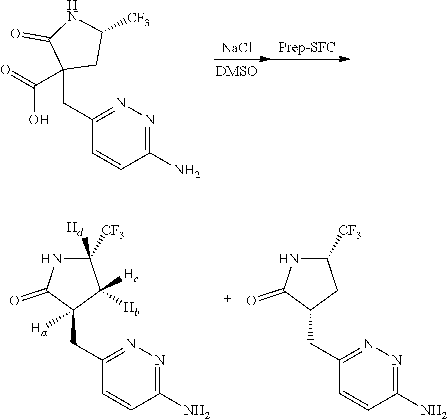

To a solution of (5S)-3-((6-aminopyridazin-3-yl)methyl)-2-oxo-5-(trifluoromethyl)pyrrolidine-3-carboxylic acid (4.30 g, 14.1 mmol, 1.00 eq, crude Li salt) in DMSO (40.0 mL) was added NaCl (1.65 g, 28.2 mmol, 2.00 eq). The reaction mixture was stirred at 120° C. for 8 h. The reaction mixture was concentrated under the vacuum to give a residue which was separated by SFC (DAICEL CHIRALCEL OX (250 mm×30 mm, 10 μm); mobile phase: [0.1% NH₃H₂O MeOH]; B %: 20%-20%, A 2.2; 70 min, RT (peak 1)=1.446 min, RT (peak 2)=1.586 min). The first eluting stereoisomer, (3S,5S)-3-((6-aminopyridazin-3-yl)methyl)-5-(trifluoromethyl)pyrrolidin-2-one (700 mg, crude), was obtained as a red solid. The relative stereochemistry was elucidated by 2D NMR, namely, utilizing HSQC δ (¹H, ¹³C): Hₐ (3.05, 39.0), H_{b} (2.3, 26.5), H_{c} (2.25, 26.5), and H_{d} (4.15, 53.0); and COSY for connectivity, showing Hₐ-H_{b}, Hₐ-H_{c}, H_{b}-H_{d}, and Hₑ-H_{d} correlations; and NOSEY for stereochemical configuration: strong NOESY cross-peaks observed between Hₐ and H_{b} as well as H_{c} and H_{d}. LCMS [M+H]⁺=260.9 m z. The second eluting stereoisomer, (3R,5S)-3-((6-aminopyridazin-3-yl)methyl)-5-(trifluoromethyl)pyrrolidin-2-one (500 mg, crude), was also obtained as a red solid with relative stereochemistry confirmed by deduction. LCMS [M+H]⁺=261.1 m/z.

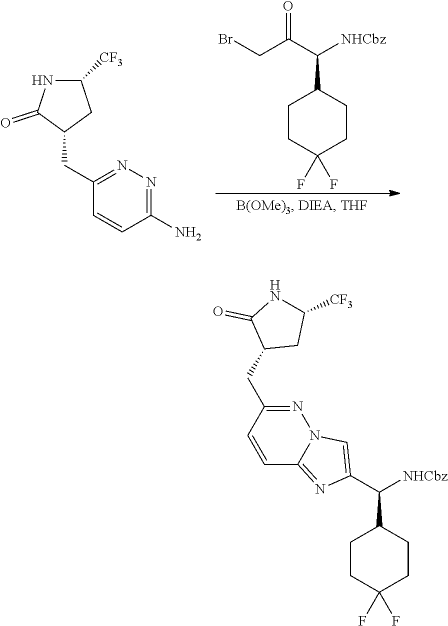 [SMILES: Nc1ccc(C[C@H]2C[C@@H](C(F)(F)F)NC2=O)nn1 ; O=C(N[C@H](C(=O)CBr)C1CCC(F)(F)CC1)OCc1ccccc1 ; O=C(N[C@H](c1cn2nc(C[C@H]3C[C@@H](C(F)(F)F)NC3=O)ccc2n1)C1CCC(F)(F)CC1)OCc1ccccc1]

To a solution of (3S,5S)-3-((6-aminopyridazin-3-yl)methyl)-5-(trifluoromethyl)pyrrolidin-2-one (200 mg, 711 μmol, 1.00 eq) in THF (1.00 mL) was added B(OMe)₃ (443 mg, 4.27 mmol, 482 μL, 6.00 eq), DIEA (551 mg, 4.27 mmol, 743 μL, 6.00 eq), and benzyl (S)-(3-bromo-1-(4,4-difluorocyclohexyl)-2-oxopropyl)carbamate (316 mg, 782 μmol, 1.10 eq) at 70° C. The reaction mixture was stirred at 70° C. for 1 h. The reaction mixture was diluted with H₂O (5.00 mL) and extracted with EtOAc (5.00 mL×3), the combined organic phase was dried over Na₂SO₄ and concentrated to give a residue. The residue was purified by prep-TLC (SiO₂, PE:EtOAc=0:1, R_{f}=0.50). The product, benzyl ((S)-(4,4-difluorocyclohexyl)(6-(((3R,5S)-2-oxo-5-(trifluoromethyl)pyrrolidin-3-yl)methyl)imidazo[1,2-b]pyridazin-2-yl)methyl)carbamate (300 mg, 530 μmol, 75% yield), was obtained as a white solid. LCMS [M+H]⁺=566.2 m/z.

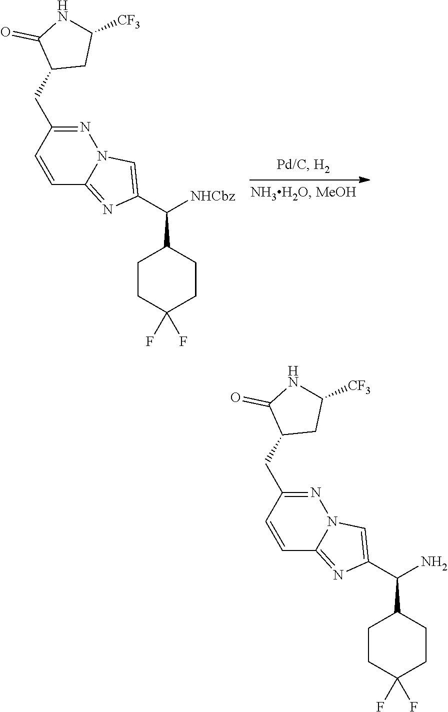 [SMILES: O=C(N[C@H](c1cn2nc(C[C@H]3C[C@@H](C(F)(F)F)NC3=O)ccc2n1)C1CCC(F)(F)CC1)OCc1ccccc1 ; N[C@H](c1cn2nc(C[C@H]3C[C@@H](C(F)(F)F)NC3=O)ccc2n1)C1CCC(F)(F)CC1]

To a solution of benzyl ((S)-(4,4-difluorocyclohexyl)(6-(((3R,5S)-2-oxo-5-(trifluoromethyl)pyrrolidin-3-yl)methyl)imidazo[1,2-b]pyridazin-2-yl)methyl)carbamate (300 mg, 530 μmol, 1.00 eq) in NH₃·H₂O (0.500 mL) and MeOH (3.00 mL) was added Pd/C (30.0 mg 10.0% purity) under N₂. The suspension was degassed under vacuum and purged with H₂ several times. The reaction mixture was stirred under H₂ (15 psi) at RT for 2 h. The reaction mixture was filtered with MeOH wash (30.0 mL) and the filtrate was concentrated to give crude (3R,5S)-3-((2-((S)-amino(4,4-difluorocyclohexyl)methyl)imidazo[1,2-b]pyridazin-6-yl)methyl)-5-(trifluoromethyl)pyrrolidin-2-one (250 mg, crude) as white oil. LCMS [M+H]⁺=432.5 m/z.

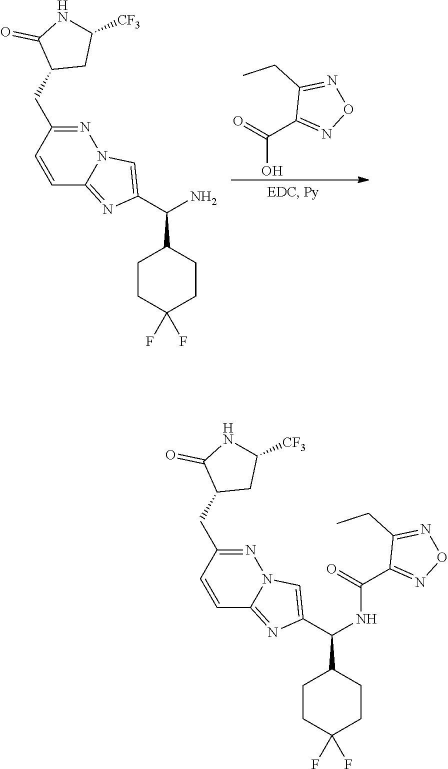

To a solution of (3R,5S)-3-((2-((S)-amino(4,4-difluorocyclohexyl)methyl)imidazo[1,2-b]pyridazin-6-yl)methyl)-5-(trifluoromethyl)pyrrolidin-2-one (80.0 mg, 185 μmol, 1.00 eq) and 4-ethyl-1,2,5-oxadiazole-3-carboxylic acid (39.5 mg, 278 μmol, 1.50 eq) in pyridine (2.00 mL) was added EDCI (71.1 mg, 370 μmol, 2.00 eq). The reaction mixture was stirred at RT for 1 h. The reaction mixture was diluted with H₂O (30.0 mL) and extracted with EtOAc (10.0 mL×3), and the combined organic phases were dried over Na₂SO₄ and concentrated to give a residue. The residue was purified by prep-TLC (SiO₂, PE:EtOAc=0:1, R_{f}=0.50). N—((S)-(4,4-difluorocyclohexyl)(6-(((3R,5S)-2-oxo-5-(trifluoromethyl)pyrrolidin-3-yl)methyl)imidazo[1,2-b]pyridazin-2-yl)methyl)-4-ethyl-1,2,5-oxadiazole-3-carboxamide (24.97 mg, 44.3 μmol, 24% yield) Compound 17 was obtained as a white solid. LCMS [M+H]⁺=556.4 m/z.

### Example 34: Preparation of Compound 18

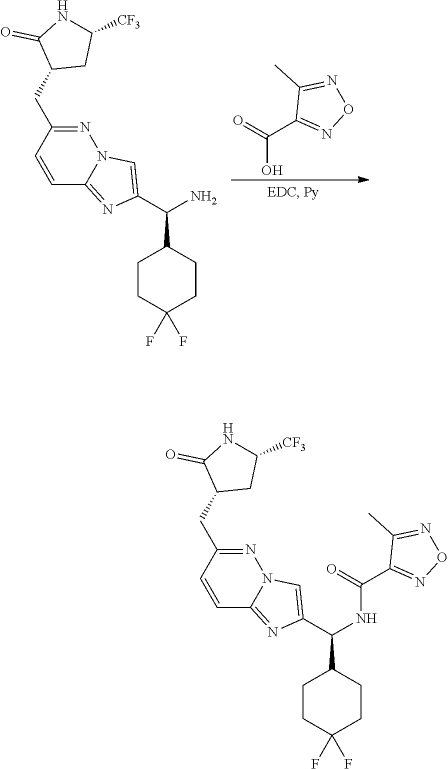

To a solution of (3R,5S)-3-((2-((S)-amino(4,4-difluorocyclohexyl)methyl)imidazo[1,2-b]pyridazin-6-yl)methyl)-5-(trifluoromethyl)pyrrolidin-2-one (80.0 mg, 185 μmol, 1.00 eq) and 4-methyl-1,2,5-oxadiazole-3-carboxylic acid (35.6 mg, 278 μmol, 1.50 eq) in pyridine (2.00 mL) was added EDCI (71.1 mg, 370 μmol, 2.00 eq). The reaction mixture was stirred at RT for 1 h. The reaction mixture was diluted with H₂O (30.0 mL) and extracted with EtOAc (10.0 mL×3), and the combined organic phases were dried over Na₂SO₄ and concentrated to give a residue. The residue was purified by prep-TLC (SiO₂, PE:EtOAc=0:1, R_{f}=0.50). Compound 18, N—((S)-(4,4-difluorocyclohexyl)(6-(((3R,5S)-2-oxo-5-(trifluoromethyl)pyrrolidin-3-yl)methyl)imidazo[1,2-b]pyridazin-2-yl)methyl)-4-methyl-1,2,5-oxadiazole-3-carboxamide (24.06 mg, 43.9 μmol, 24% yield), was obtained as a white solid. LCMS [M+H]⁺=542.2 m/z.

### Example 35: Preparation of Compound 19

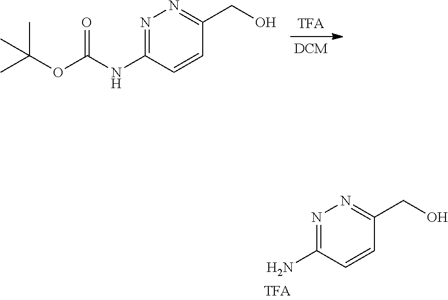 [SMILES: CC(C)(C)OC(=O)Nc1ccc(CO)nn1 ; Nc1ccc(CO)nn1]

To a solution of tert-butyl (6-(hydroxymethyl)pyridazin-3-yl)carbamate (110 g, 488.36 mmol, 1.00 eq) in DCM (1100 mL) was added TFA (445.47 g, 3.91 μmol, 289.27 mL, 8.00 eq) at RT. The reaction mixture was stirred at RT for 12 h. The reaction mixture was combined with that of two other reactions (155 mmol and 488 mmol reactions) for work-up. The combined reaction mixture was concentrated under reduced pressure to give the crude product. The crude product was used into the next step without further purification. Crude product, (6-aminopyridazin-3-yl) MeOH (502 g, crude, TFA salt), was obtained as a brown solid. LCMS [M+H]⁺=126.2 m/z.

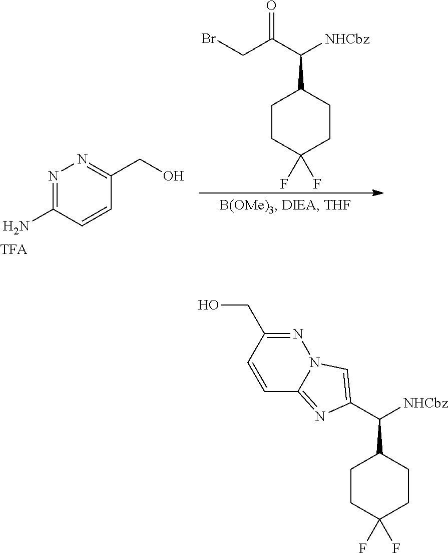 [SMILES: Nc1ccc(CO)nn1 ; O=C(N[C@H](C(=O)CBr)C1CCC(F)(F)CC1)OCc1ccccc1 ; O=C(N[C@H](c1cn2nc(CO)ccc2n1)C1CCC(F)(F)CC1)OCc1ccccc1]

A mixture of (6-aminopyridazin-3-yl) MeOH (75.0 g, 81.5 mmol, 26% purity, 1.00 eq, TFA salt), benzyl (S)-(3-bromo-1-(4,4-difluorocyclohexyl)-2-oxopropyl)carbamate (29.67 g, 73.38 mmol, 0.90 eq), B(OMe)₃ (16.95 g, 163.08 mmol, 18.42 mL, 2.00 eq), DIEA (84.31 g 652.31 mmol, 113.62 mL, 8.00 eq) in THF (800 mL) was degassed and purged with N₂ 3 times, and then the reaction mixture was stirred at 75° C. for 12 h under N₂ atmosphere. The reaction mixture on was combined with a second 81.5 mmol scale reaction for work-up. The combined reaction mixture was diluted with H₂O (2000 mL) and extracted with EA (1500 mL×3). The combined organic layers were washed with brine 1000 mL, dried over Na₂SO₄, filtered, and concentrated under reduced pressure to give a residue. The crude product was combined with 13.0 g crude material from a separate batch. The combined residue was purified by flash silica gel chromatography (ISCO®; 130 g SepaFlash® Silica Flash Column, Eluent of 0-50% EtOAc/PE gradient @100 mL/min). Desired product, benzyl (S)-((4,4-difluorocyclohexyl)(6-(hydroxymethyl)imidazo[1,2-b]pyridazin-2-yl)methyl)carbamate (50.7 g), was obtained as a yellow solid. LCMS [M+H]⁺=431.1 m/z.

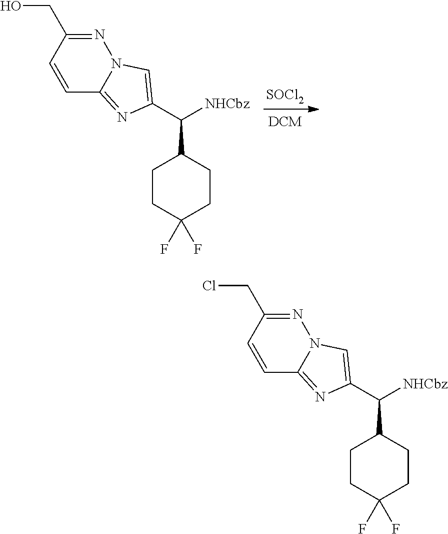

To a solution of benzyl (S)-((4,4-difluorocyclohexyl)(6-(hydroxymethyl)imidazo[1,2-b]pyridazin-2-yl)methyl)carbamate (1.50 g, 3.48 mmol, 1.00 eq) in DCM (20.0 mL) was added SOCl₂ (6.60 g, 55.4 mmol, 4.03 mL, 15.9 eq) at 0° C. The reaction mixture was stirred at RT for 1 h. The reaction mixture was concentrated under reduced pressure to give a residue. The product, benzyl (S)-((6-(chloromethyl)imidazo[1,2-b]pyridazin-2-yl)(4,4-difluorocyclohexyl)methyl)carbamate (1.55 g, 3.45 mmol, 99% crude yield), was obtained as a brown solid. LCMS [M+H]⁺=449.2 m/z.

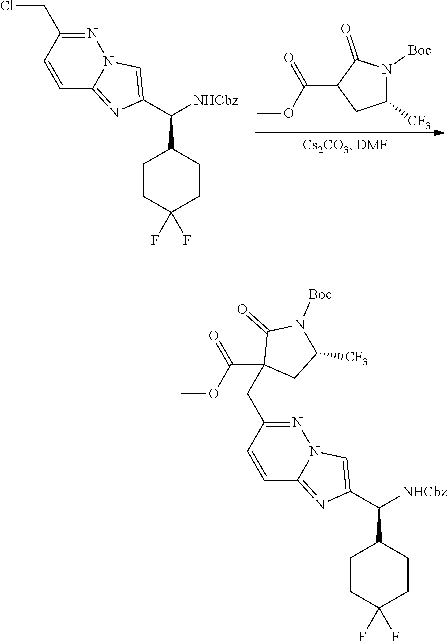 [SMILES: O=C(N[C@H](c1cn2nc(CCl)ccc2n1)C1CCC(F)(F)CC1)OCc1ccccc1 ; COC(=O)C1C[C@@H](C(F)(F)F)N(C(=O)OC(C)(C)C)C1=O ; COC(=O)C1(Cc2ccc3nc([C@@H](NC(=O)OCc4ccccc4)C4CCC(F)(F)CC4)cn3n2)C[C@@H](C(F)(F)F)N(C(=O)OC(C)(C)C)C1=O]

To solution of 1-(tert-butyl) 3-methyl (5S)-2-oxo-5-(trifluoromethyl)pyrrolidine-1,3-dicarboxylate (832 mg, 2.67 mmol, 2.00 eq) and Cs₂CO₃ (2.18 g, 6.68 mmol, 5.00 eq) in DMF (5.00 mL). The reaction mixture was stirred at RT for 30 min then benzyl (S)-((6-(chloromethyl)imidazo[1,2-b]pyridazin-2-yl)(4,4-difluorocyclohexyl)methyl)carbamate (600 mg, 1.34 mmol, 1.00 eq) was added. The reaction mixture was stirred at RT for 5 h. The reaction mixture was concentrated under reduced pressure to remove solvent, then diluted with H₂O (15.0 mL) and extracted with EtOAc (10.0 mL×3). The organic layers were dried over Na₂SO₄, filtered, and concentrated under reduced pressure to give a residue. The residue was purified by prep-TLC (DCM:MeOH=10:1). The product, 1-(tert-butyl) 3-methyl (5S)-3-((2-((S)-(((benzyloxy)carbonyl)amino)(4,4-difluorocyclohexyl)methyl)imidazo[1,2-b]pyridazin-6-yl)methyl)-2-oxo-5-(trifluoromethyl)pyrrolidine-1,3-dicarboxylate (250 mg, 345.45 μmol, 26% yield), was obtained as a brown solid. LCMS [M+H]⁺=724.4 m/z.

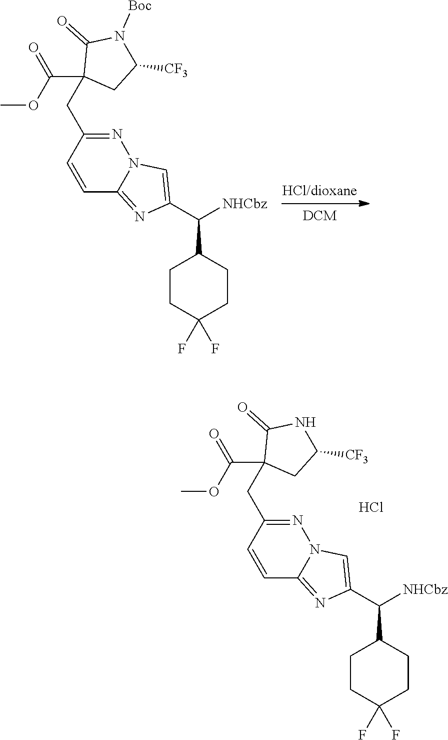 [SMILES: COC(=O)C1(Cc2ccc3nc([C@@H](NC(=O)OCc4ccccc4)C4CCC(F)(F)CC4)cn3n2)C[C@@H](C(F)(F)F)N(C(=O)OC(C)(C)C)C1=O ; COC(=O)C1(Cc2ccc3nc([C@@H](NC(=O)OCc4ccccc4)C4CCC(F)(F)CC4)cn3n2)C[C@@H](C(F)(F)F)NC1=O ; Cl]

To a solution of 1-(tert-butyl) 3-methyl (5S)-3-((2-((S)-(((benzyloxy)carbonyl)amino)(4,4-difluorocyclohexyl)methyl)imidazo[1,2-b]pyridazin-6-yl)methyl)-2-oxo-5-(trifluoromethyl)pyrrolidine-1,3-dicarboxylate (250 mg, 345 μmol, 1.00 eq) in DCM (3.00 mL) was added HCl/dioxane (4 M, 86.3 μL, 1.00 eq) at 0° C. The reaction mixture was stirred at RT for 1 h. The reaction mixture was concentrated under reduced pressure to dryness. Methyl (5S)-3-((2-((S)-(((benzyloxy)carbonyl)amino)(4,4-difluorocyclohexyl)methyl)imidazo[1,2-b]pyridazin-6-yl)methyl)-2-oxo-5-(trifluoromethyl)pyrrolidine-3-carboxylate (200 mg, 303 μmol, 88% yield, HCl salt) was obtained as a yellow solid. LCMS [M+H]⁺=624.3 m/z.

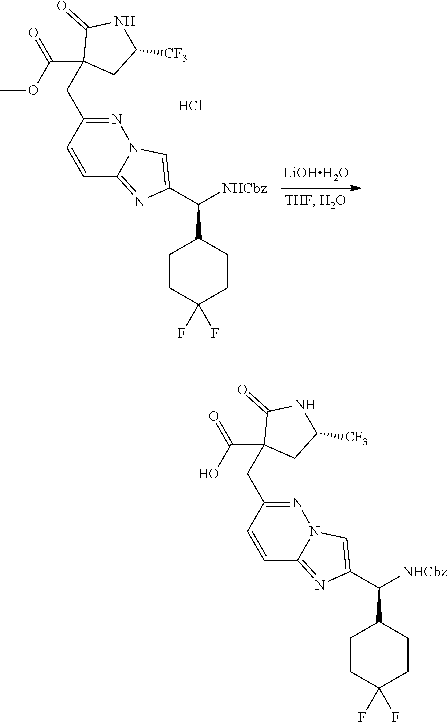 [SMILES: COC(=O)C1(Cc2ccc3nc([C@@H](NC(=O)OCc4ccccc4)C4CCC(F)(F)CC4)cn3n2)C[C@@H](C(F)(F)F)NC1=O ; Cl ; "LiOH·H2O" ; O=C(N[C@H](c1cn2nc(CC3(C(=O)O)C[C@@H](C(F)(F)F)NC3=O)ccc2n1)C1CCC(F)(F)CC1)OCc1ccccc1]

To a solution of methyl (5S)-3-((2-((S)-(((benzyloxy)carbonyl)amino)(4,4-difluorocyclohexyl)methyl)imidazo[1,2-b]pyridazin-6-yl)methyl)-2-oxo-5-(trifluoromethyl)pyrrolidine-3-carboxylate (200 mg, 303 μmol, 1.00 eq, HCl salt) in THE (1.00 mL) was added LiOH·H₂O (63.5 mg, 1.52 mmol, 5.00 eq) and H₂O (5.46 mg, 303 μmol, 5.46 μL, 1.00 eq). The reaction mixture was stirred at RT for 2 h. The reaction mixture was adjusted pH=7 with HCl (1 M) and was diluted with H₂O (10.0 mL) and extracted with EtOAc (10.0 mL×3). Organic layers were combined, dried over Na₂SO₄, filtered, and concentrated under reduced pressure to give a residue. Crude (5S)-3-((2-((S)-(((benzyloxy)carbonyl)amino)(4,4-difluorocyclohexyl)methyl)imidazo[1,2-b]pyridazin-6-yl)methyl)-2-oxo-5-(trifluoromethyl)pyrrolidine-3-carboxylic acid (180 mg, 295.3 μmol, 97% crude yield) was obtained as a light-yellow solid. LCMS [M+H]⁺=610.2 m/z.

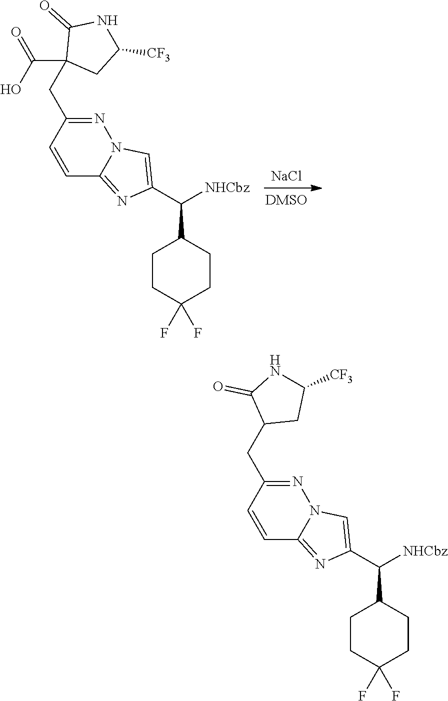 [SMILES: O=C(N[C@H](c1cn2nc(CC3(C(=O)O)C[C@@H](C(F)(F)F)NC3=O)ccc2n1)C1CCC(F)(F)CC1)OCc1ccccc1 ; "NaCl" ; O=C(N[C@H](c1cn2nc(CC3C[C@@H](C(F)(F)F)NC3=O)ccc2n1)C1CCC(F)(F)CC1)OCc1ccccc1]

To a solution of crude (5S)-3-((2-((S)-(((benzyloxy)carbonyl)amino)(4,4-difluorocyclohexyl)methyl)imidazo[1,2-b]pyridazin-6-yl)methyl)-2-oxo-5-(trifluoromethyl)pyrrolidine-3-carboxylic acid (180 mg, 295.3 μmol, 1.00 eq, crude) in DMF (3.00 mL) was added NaCl (34.5 mg, 590 μmol, 2.00 eq). The reaction mixture was stirred at 120° C. for 1 h. The reaction mixture was diluted with H₂O (5.00 mL) and extracted with EtOAc (10.0 mL×3). The organic layers were combined, dried over Na₂SO₄, filtered, and concentrated under reduced pressure to give a residue. The residue was purified by prep-TLC (DCM:MeOH=10:1). Benzyl ((1S)-(4,4-difluorocyclohexyl)(6-(((5S)-2-oxo-5-(trifluoromethyl)pyrrolidin-3-yl)methyl)imidazo[1,2-b]pyridazin-2-yl)methyl)carbamate (70.0 mg, 123.78 μmol, 42% yield) was obtained as a white solid. LCMS [M+H]⁺=566.3 m/z.

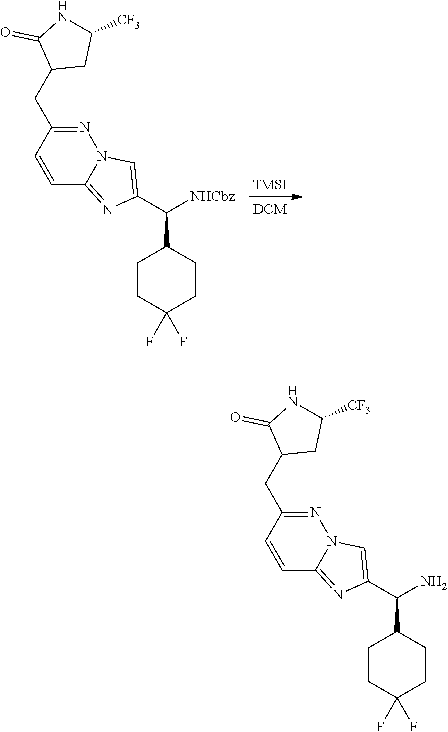 [SMILES: "TMSI" ; O=C(N[C@H](c1cn2nc(CC3C[C@@H](C(F)(F)F)NC3=O)ccc2n1)C1CCC(F)(F)CC1)OCc1ccccc1 ; N[C@H](c1cn2nc(CC3C[C@@H](C(F)(F)F)NC3=O)ccc2n1)C1CCC(F)(F)CC1]

To a solution of benzyl ((1S)-(4,4-difluorocyclohexyl)(6-(((5S)-2-oxo-5-(trifluoromethyl)pyrrolidin-3-yl)methyl)imidazo[1,2-b]pyridazin-2-yl)methyl)carbamate (65.0 mg, 114 μmol, 1.00 eq) in DCM (1.00 mL) was added TMSI (46.0 mg, 229 μmol, 31.2 μL, 2.00 eq) at 0° C. The reaction mixture was stirred at RT for 1 h. The reaction mixture was concentrated under reduced pressure to remove DCM. The residue was diluted with HCl (1 M, 5.00 mL) and washed with EtOAc (5.00 mL×3). The aqueous phase was adjusted to pH=9 with saturated aqueous Na₂CO₃ and extracted with EtOAc (5.00 mL×3). These organic layers were dried over Na₂SO₄, filtered, and concentrated under reduced pressure to give a residue. The product, (5S)-3-((2-((S)-amino(4,4-difluorocyclohexyl)methyl)imidazo[1,2-b]pyridazin-6-yl)methyl)-5-(trifluoromethyl)pyrrolidin-2-one (40.0 mg, 92.7 μmol, 81% yield), was obtained as a white solid. LCMS [M+H]⁺=432.1 m/z.

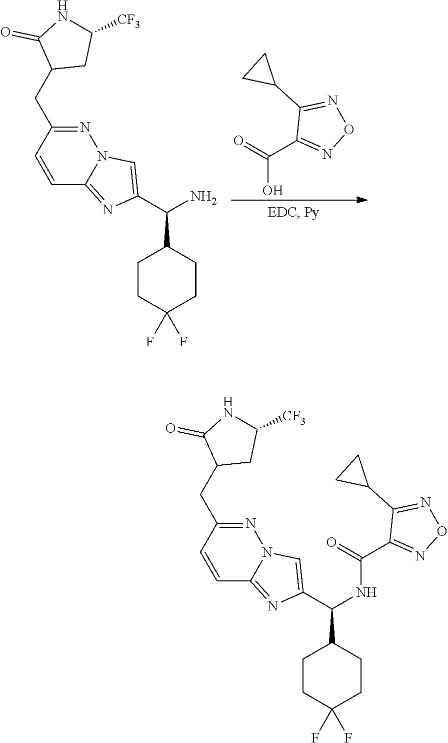 [SMILES: N[C@H](c1cn2nc(CC3C[C@@H](C(F)(F)F)NC3=O)ccc2n1)C1CCC(F)(F)CC1 ; O=C(O)c1nonc1C1CC1 ; O=C(N[C@H](c1cn2nc(CC3C[C@@H](C(F)(F)F)NC3=O)ccc2n1)C1CCC(F)(F)CC1)c1nonc1C1CC1]

To a solution of (5S)-3-((2-((S)-amino(4,4-difluorocyclohexyl)methyl)imidazo[1,2-b]pyridazin-6-yl)methyl)-5-(trifluoromethyl)pyrrolidin-2-one (35.0 mg, 81.1 μmol, 1.00 eq) and 4-cyclopropyl-1,2,5-oxadiazole-3-carboxylic acid (18.7 mg, 121 μmol, 1.50 eq) in pyridine (1.50 mL) was added EDCI (46.6 mg, 243 μmol, 3.00 eq). The reaction mixture was stirred at RT for 1 h. The reaction mixture was diluted with H₂O 5.00 mL and extracted with EtOAc (5.00 mL×3). The organic layers were combined, dried over Na₂SO₄, filtered, and concentrated under reduced pressure to give a residue. The residue was purified by prep-HPLC (UniSil 3-100 C₁₈ Ultra (150×25 mm×3 μm); mobile phase: [water (FA)-ACN]; gradient: 53%-73% B over 7 min). Crude 4-cyclopropyl-N-((1S)-(4,4-difluorocyclohexyl)(6-(((5S)-2-oxo-5-(trifluoromethyl)pyrrolidin-3-yl)methyl)imidazo[1,2-b]pyridazin-2-yl)methyl)-1,2,5-oxadiazole-3-carboxamide (18.0 mg, 31.72 μmol, 39% yield) as a mixture of stereoisomers was obtained as a white solid. LCMS [M+H]⁺=568.2 m/z.

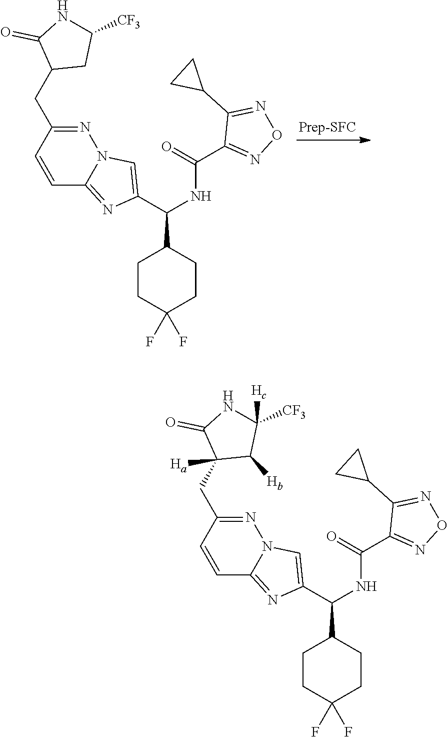

The mixture of stereoisomers (18.0 mg, 31.72 μmol) was purified by SFC (DAICEL CHIRALCEL OD (250 mm×30 mm, 10 μm); mobile phase: [CO₂-MeOH (0.1% NH₃H₂O)]; 25% B isocratic elution mode). Compound 19, 4-cyclopropyl-N—((S)-(4,4-difluorocyclohexyl)(6-(((3R,5S)-2-oxo-5-(trifluoromethyl)pyrrolidin-3-yl)methyl)imidazo[1,2-b]pyridazin-2-yl)methyl)-1,2,5-oxadiazole-3-carboxamide (8.82 mg, 15.49 μmol, 49% yield), was the second eluting stereoisomer, obtained as a white solid. The relative stereochemistry was elucidated by 2D NMR, namely, utilizing HSQC δ (¹H, ¹³C): Hₐ (3.05, 39.0), H_{b} (2.60, 26.5), and H_{c} (4.05, 53.5); and COSY for connectivity, showing Hₐ-H_{b} and H_{b}-H_{c} correlations; and NOSEY for stereochemical configuration: NOESY cross-peaks observed between Hₐ and H_{b} as well as H_{b} and H_{c}. LCMS [M+H]⁺=568.3 m/z.

### Example 36: Preparation of Compound 20

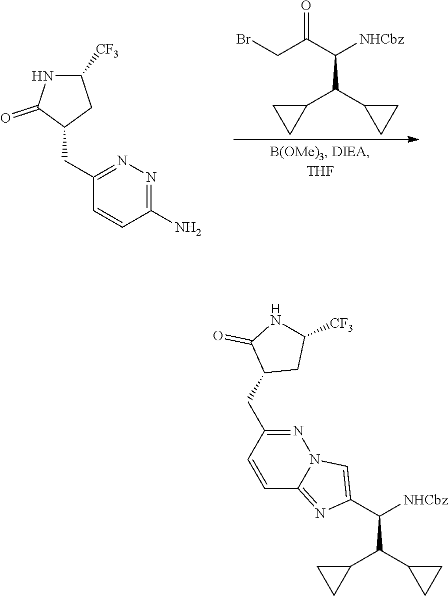 [SMILES: Nc1ccc(C[C@H]2C[C@@H](C(F)(F)F)NC2=O)nn1 ; O=C(N[C@H](C(=O)CBr)C(C1CC1)C1CC1)OCc1ccccc1 ; O=C(N[C@H](c1cn2nc(C[C@H]3C[C@@H](C(F)(F)F)NC3=O)ccc2n1)C(C1CC1)C1CC1)OCc1ccccc1]

To a solution of (3R,5S)-3-((6-aminopyridazin-3-yl)methyl)-5-(trifluoromethyl)pyrrolidin-2-one (200 mg, 768 μmol, 1.00 eq), benzyl (S)-(4-bromo-1,1-dicyclopropyl-3-oxobutan-2-yl)carbamate (350 mg, 922 μmol, 1.20 eq) in THE (3.00 mL) was added B(OMe)₃ (479 mg, 4.61 mmol, 520 μL, 6.00 eq) and DIEA (596 mg, 4.61 mmol, 803 μL, 6.00 eq). The reaction mixture was stirred at 70° C. for 2 h. The reaction mixture was diluted with water (40.0 mL) and extracted with EtOAc (40.0 mL×2). The combined organic layers were washed with brine (40.0 mL), dried over Na₂SO₄, filtered, and concentrated under reduced pressure to give a residue. The residue was purified by prep-TLC (SiO₂, PE:EtOAc=0:1, R_{f}=0.39). Benzyl ((S)-2,2-dicyclopropyl-1-(6-(((3R,5S)-2-oxo-5-(trifluoromethyl)pyrrolidin-3-yl)methyl)imidazo[1,2-b]pyridazin-2-yl)ethyl)carbamate (200 mg, 369 μmol, 48% yield) was obtained as a yellow oil. LCMS [M+H]⁺=542.4 m/z.

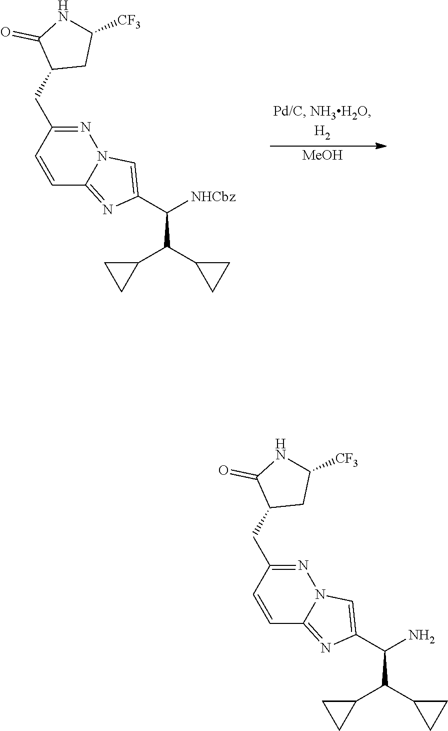 [SMILES: "MeOH" ; O=C(N[C@H](c1cn2nc(C[C@H]3C[C@@H](C(F)(F)F)NC3=O)ccc2n1)C(C1CC1)C1CC1)OCc1ccccc1 ; N[C@H](c1cn2nc(C[C@H]3C[C@@H](C(F)(F)F)NC3=O)ccc2n1)C(C1CC1)C1CC1]

To a solution of benzyl ((S)-2,2-dicyclopropyl-1-(6-(((3R,5S)-2-oxo-5-(trifluoromethyl)pyrrolidin-3-yl)methyl)imidazo[1,2-b]pyridazin-2-yl)ethyl)carbamate (200 mg 369 μmol, 1.00 eq) in MeOH (4.00 mL) was added Pd/C (30.0 mg, 10.0% purity) and NH₃·H₂O (364 mg, 2.60 mmol, 0.400 mL, 25.0% purity, 7.03 eq), and the reaction mixture was stirred at RT for 1 h under H₂ (15 psi) atmosphere. The reaction mixture was filtered, and the filtrate was concentrated under vacuum. (3R,5S)-3-((2-((S)-1-amino-2,2-dicyclopropylethyl)imidazo[1,2-b]pyridazin-6-yl)methyl)-5-(trifluoromethyl)pyrrolidin-2-one (140 mg, 343 μmol, 93% yield) was obtained as a yellow solid. LCMS [M+H]⁺=408.3 m/z.

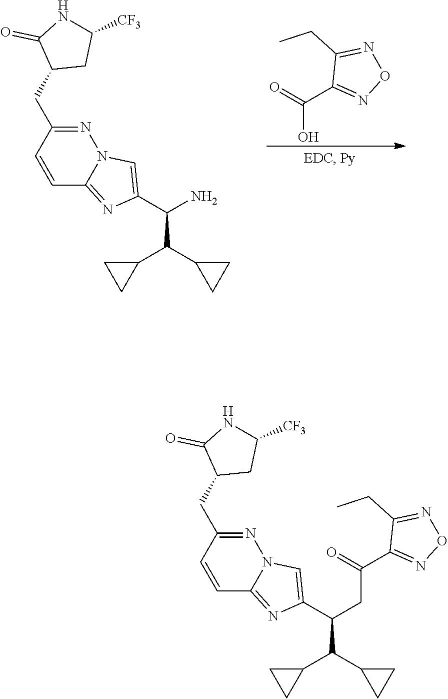

To a solution of (3R,5S)-3-((2-((S)-1-amino-2,2-dicyclopropylethyl)imidazo[1,2-b]pyridazin-6-yl)methyl)-5-(trifluoromethyl)pyrrolidin-2-one (65.0 mg, 159 μmol, 1.00 eq), 4-ethyl-1,2,5-oxadiazole-3-carboxylic acid (34.0 mg, 239 μmol, 1.50 eq) in pyridine (2.00 mL) was added EDCI (122 mg, 638 μmol, 4.00 eq). The reaction mixture was stirred at RT for 2 h. The reaction mixture was diluted with H₂O (20.0 mL) and extracted with DCM (20.0 mL×3), the combined organic layers were dried over Na₂SO₄ and concentrated to give a residue. The residue was purified by prep-TLC (SiO₂, PE:EtOAc=0:1, R_{f}=0.29). Compound 20, N—((S)-2,2-dicyclopropyl-1-(6-(((3R,5S)-2-oxo-5-(trifluoromethyl)pyrrolidin-3-yl)methyl)imidazo[1,2-b]pyridazin-2-yl)ethyl)-4-ethyl-1,2,5-oxadiazole-3-carboxamide, (24.37 mg, 45.3 μmol, 28% yield) was obtained as a white solid. LCMS [M+H]⁺=532.4 m/z.

### Example 37: Preparation of Compound 21

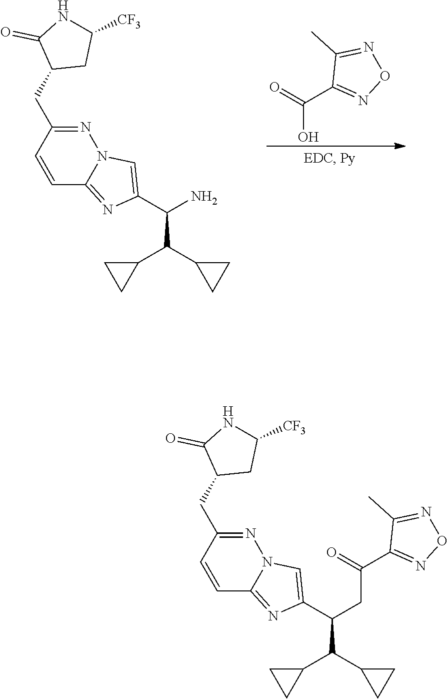

To a solution of (3R,5S)-3-((2-((S)-1-amino-2,2-dicyclopropylethyl)imidazo[1,2-b]pyridazin-6-yl)methyl)-5-(trifluoromethyl)pyrrolidin-2-one (60.0 mg, 147 μmol, 1.00 eq) and 4-methyl-1,2,5-oxadiazole-3-carboxylic acid (28.3 mg, 221 μmol, 1.50 eq) in pyridine (2.00 mL) was added EDCI (113 mg, 589 μmol, 4.00 eq). The reaction mixture was stirred at RT for 2 h. The reaction mixture was diluted with H₂O (20.0 mL) and extracted with DCM (20.0 mL×3). The combined organic layers were dried over Na₂SO₄ and concentrated to give a residue. The residue was purified by prep-TLC (SiO₂, PE:EtOAc=0:1, R_{f}=0.37). Compound 21, N—((S)-2,2-dicyclopropyl-1-(6-(((3R,5S)-2-oxo-5-(trifluoromethyl)pyrrolidin-3-yl)methyl)imidazo[1,2-b]pyridazin-2-yl)ethyl)-4-methyl-1,2,5-oxadiazole-3-carboxamide (24.56 mg, 46.9 μmol, 32% yield), was obtained as a white solid. LCMS [M+H]⁺=518.5 m/z.

### Example 38: Preparation of Compound 22

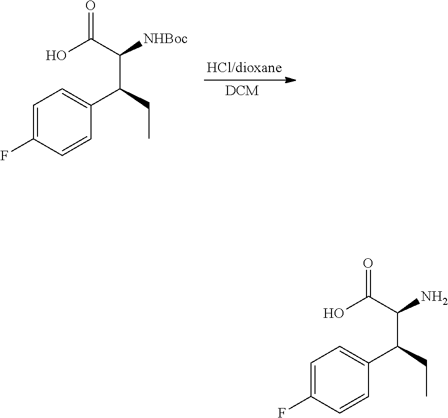 [SMILES: CC[C@@H](c1ccc(F)cc1)[C@H](NC(=O)OC(C)(C)C)C(=O)O ; CC[C@@H](c1ccc(F)cc1)[C@H](N)C(=O)O]

To a solution of (2S,3S)-2-((tert-butoxycarbonyl)amino)-3-(4-fluorophenyl)pentanoic acid (4.80 g, 15.4 mmol, 1.00 eq) in DCM (50.0 mL) was added HCl/dioxane (4.00 M, 20.0 mL, 5.19 eq). The reaction mixture was stirred at RT for 1 h. The reaction mixture was concentrated under reduced pressure to give a residue. The product, (2S,3S)-2-amino-3-(4-fluorophenyl)pentanoic acid (3.50 g, crude HCl salt), was obtained as a yellow solid. LCMS [M+H]⁺=212.1 m/z.

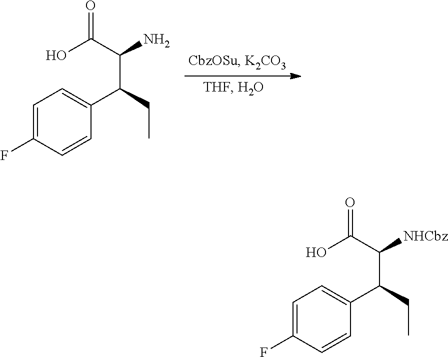 [SMILES: CC[C@@H](c1ccc(F)cc1)[C@H](N)C(=O)O ; CC[C@@H](c1ccc(F)cc1)[C@H](NC(=O)OCc1ccccc1)C(=O)O]

To a solution of (2S,3S)-2-amino-3-(4-fluorophenyl)pentanoic acid (3.50 g, crude HCl salt) in THF (30.0 mL), H₂O (30.0 mL) was added CbzOSu (4.13 g, 16.5 mmol, 1.00 eq) and K₂CO₃ (6.87 g, 49.7 mmol, 3.00 eq). The reaction mixture was stirred at RT for 12 h. The reaction mixture was acidized to pH=5 with HCl (1.00 M) and extracted with DCM (60.0 mL×3). The combined organic layers were washed brine (60.0 mL×2) and concentrated under reduced pressure to give a residue. The residue was purified by column chromatography (SiO₂, PE:EtOAc=1:0 to 1:1). The product, (2S,3S)-2-(((benzyloxy)carbonyl)amino)-3-(4-fluorophenyl)pentanoic acid (5.00 g, 14.4 mmol, 94% yield over 2 steps), was obtained as a white solid. LCMS [M+Na]⁺=368.1 m/z.

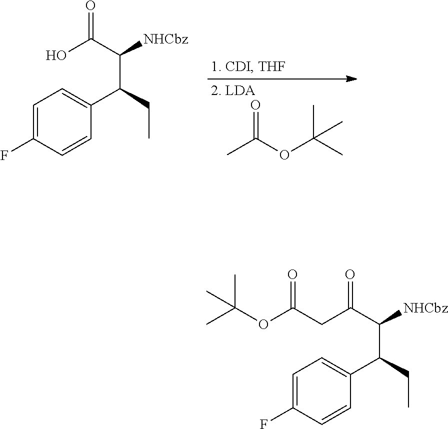

To a solution of (2S,3S)-2-(((benzyloxy)carbonyl)amino)-3-(4-fluorophenyl)pentanoic acid (2.00 g, 5.79 mmol, 1.00 eq) in THF (20.0 mL) was added CDI (1.13 g, 6.95 mmol, 1.20 eq) at 0° C. and the resulting mixture was stirred at 0° C. for 2 h. Meanwhile, to a solution of tert-butyl acetate (2.02 g, 17.4 mmol, 2.33 mL, 3.00 eq) in THF (20.0 mL) was added LDA (2.00 M, 8.69 mL, 3.00 eq) at −78° C. and the reaction mixture was stirred at −78° C. for 2 h. The residue was diluted with saturated aqueous NH₄Cl (40.0 mL) and extracted with EtOAc (60.0 mL×3). Combined organic layers were dried over Na₂SO₄, filtered and concentrated under reduced pressure to give a residue. The residue was purified by column chromatography (SiO₂, PE:EtOAc=1:0 to 5:1). The product, tert-butyl (4S,5S)-4-(((benzyloxy)carbonyl)amino)-5-(4-fluorophenyl)-3-oxoheptanoate (1.60 g, 3.61 mmol, 62% yield), was obtained as a yellow oil. LCMS [M+Na]⁺=466.3 m/z.

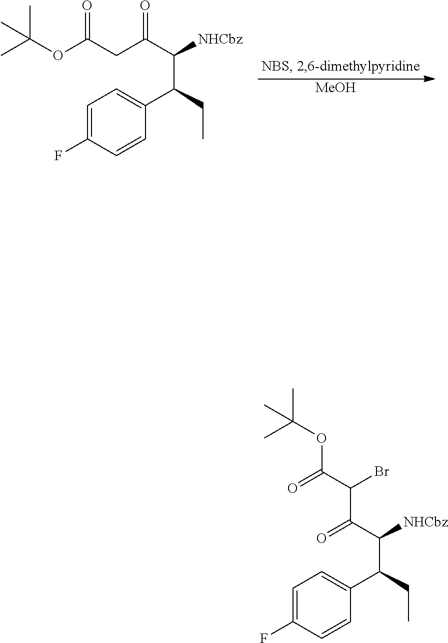 [SMILES: "MeOH" ; CC[C@@H](c1ccc(F)cc1)[C@H](NC(=O)OCc1ccccc1)C(=O)CC(=O)OC(C)(C)C ; CC[C@@H](c1ccc(F)cc1)[C@H](NC(=O)OCc1ccccc1)C(=O)C(Br)C(=O)OC(C)(C)C]

To a solution of tert-butyl (4S,5S)-4-(((benzyloxy)carbonyl)amino)-5-(4-fluorophenyl)-3-oxoheptanoate (1.60 g, 3.61 mmol, 1.00 eq) in MeOH (16.0 mL) was added NBS (513 mg, 2.89 mmol, 0.800 eq) and 2,6-dimethylpyridine (30.9 mg, 288 μmol, 33.6 μL, 0.0800 eq). The reaction mixture was stirred at RT for 1 h. The reaction mixture was concentrated under reduced pressure to give a residue. The product, tert-butyl (4S,5S)-4-(((benzyloxy)carbonyl)amino)-2-bromo-5-(4-fluorophenyl)-3-oxoheptanoate (1.80 g, crude), was obtained as a yellow oil. LCMS [M+Na]⁺=540.9 m/z.

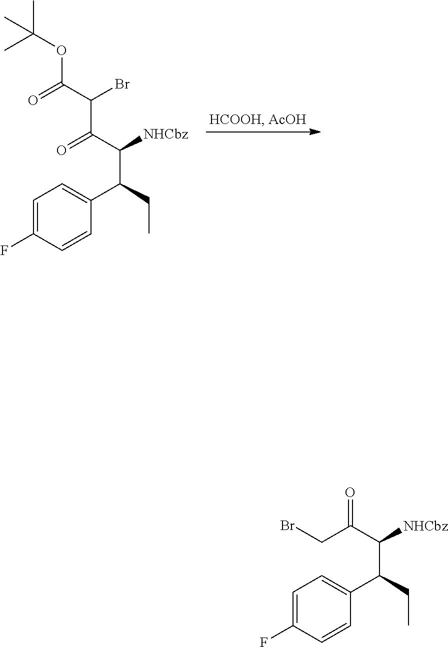 [SMILES: CC[C@@H](c1ccc(F)cc1)[C@H](NC(=O)OCc1ccccc1)C(=O)C(Br)C(=O)OC(C)(C)C ; CC[C@@H](c1ccc(F)cc1)[C@H](NC(=O)OCc1ccccc1)C(=O)CBr]

A solution of tert-butyl (4S,5S)-4-(((benzyloxy)carbonyl)amino)-2-bromo-5-(4-fluorophenyl)-3-oxoheptanoate (1.80 g, crude) in HCOOH (165 mg, 3.45 mmol, 5.00 mL, 1.00 eq) and AcOH (9.45 g, 157 mmol, 9.00 mL, 45.6 eq) was stirred at 80° C. for 1 h. The residue was diluted with saturated aqueous NaHCO₃ to pH=7 and extracted with EtOAc (80.0 mL×3). Combined organic layers were dried over Na₂SO₄, filtered, and concentrated under reduced pressure to give a residue. The residue was purified by column chromatography (SiO₂, PE:EtOAc=1:0 to 5:1). Benzyl ((3S,4S)-1-bromo-4-(4-fluorophenyl)-2-oxohexan-3-yl)carbamate (1.00 g 2.37 mmol, 66% yield over 2 steps) was obtained as a yellow oil. LCMS [M+H]⁺=422.1 m/z.

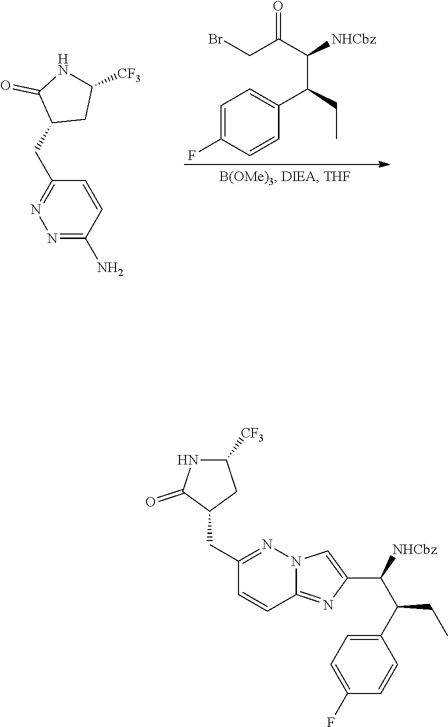

To a solution of benzyl ((3S,4S)-1-bromo-4-(4-fluorophenyl)-2-oxohexan-3-yl)carbamate (110 mg, 260 μmol, 1.00 eq) and (3R,5S)-3-((6-aminopyridazin-3-yl)methyl)-5-(trifluoromethyl)pyrrolidin-2-one (33.9 mg, 130 μmol, 0.500 eq) in THF (2.00 mL) was added B(OMe)₃ (162 mg, 1.56 mmol, 177 μL, 6.00 eq) and DIEA (202 mg, 1.56 mmol, 272 μL, 6.00 eq). The reaction mixture was stirred at 70° C. for 2 h. The reaction mixture was diluted with H₂O (100 mL) and extracted with a mixture of DCM and MeOH (DCM:MeOH=10:1, 100 mL×3). The combined organic phases were dried over Na₂SO₄ and concentrated to give a residue. The residue was purified by prep-TLC (PE:EtOAc=0:1, R_{f}=0.36). Benzyl ((1S,2S)-2-(4-fluorophenyl)-1-(6-(((3R,5S)-2-oxo-5-(trifluoromethyl)pyrrolidin-3-yl)methyl)imidazo[1,2-b]pyridazin-2-yl)butyl)carbamate (80.0 mg, 137 μmol, 53% yield) was obtained as a yellow solid. LCMS [M+H]⁺=584.3 m/z.

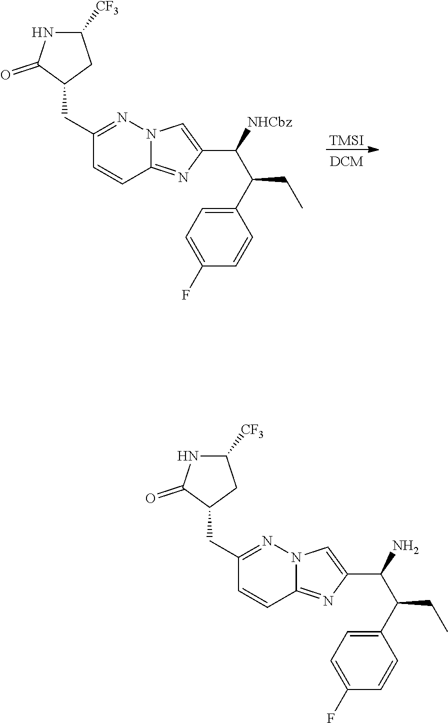 [SMILES: "TMSI" ; CC[C@@H](c1ccc(F)cc1)[C@H](NC(=O)OCc1ccccc1)c1cn2nc(C[C@H]3C[C@@H](C(F)(F)F)NC3=O)ccc2n1 ; CC[C@@H](c1ccc(F)cc1)[C@H](N)c1cn2nc(C[C@H]3C[C@@H](C(F)(F)F)NC3=O)ccc2n1]

A solution of benzyl ((1S,2S)-2-(4-fluorophenyl)-1-(6-(((3R,5S)-2-oxo-5-(trifluoromethyl)pyrrolidin-3-yl)methyl)imidazo[1,2-b]pyridazin-2-yl)butyl)carbamate (70.0 mg 120 μmol, 1.00 eq) in DCM (2.00 mL) was added TMSI (72.0 mg, 360 μmol, 49.0 μL, 3.00 eq) at 0° C., then the reaction mixture was stirred at RT for 2 h. The reaction mixture was diluted with water (30.0 mL) and adjusted with HCl (1.00 M) to pH=3, then washed with EtOAc (20.0 mL×2). The aqueous phase was adjusted pH to 9 with saturated aqueous NaHCO₃ and extracted with EtOAc (20.0 mL×3). The combined organic layers were dried over anhydrous Na₂SO₄, filtered, and concentrated under reduced pressure to give a residue. The product, (3R,5S)-3-((2-((1S,2S)-1-amino-2-(4-fluorophenyl)butyl)imidazo[1,2-b]pyridazin-6-yl)methyl)-5-(trifluoromethyl)pyrrolidin-2-one (30.0 mg, 66.8 μmol, 56% yield), was obtained as a yellow oil. LCMS [M+H]⁺=450.2 m/z.

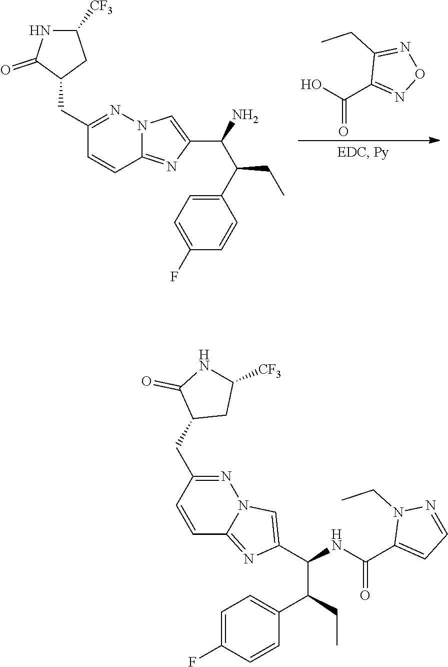

A solution of (3R,5S)-3-((2-((1S,2S)-1-amino-2-(4-fluorophenyl)butyl)imidazo[1,2-b]pyridazin-6-yl)methyl)-5-(trifluoromethyl)pyrrolidin-2-one (25.0 mg, 55.6 μmol, 1.00 eq) and 1-ethyl-1H-pyrazole-5-carboxylic acid (23.4 mg, 167 μmol, 3.00 eq) in pyridine (1.00 mL) was added EDCI (21.3 mg, 111 μmol, 2.00 eq), the reaction mixture was stirred at RT for 2 h. The reaction mixture was diluted with H₂O (50.0 mL) and extracted with a mixture of DCM and MeOH (DCM:MeOH=10:1, 50.0 mL×3). The combined organic phases were dried over Na₂SO₄ and concentrated to give a residue. The residue was purified by prep-TLC (DCM:MeOH=10:1, R_{f}=0.50). Compound 22, 1-ethyl-N-((1S,2S)-2-(4-fluorophenyl)-1-(6-(((3R,5S)-2-oxo-5-(trifluoromethyl)pyrrolidin-3-yl)methyl)imidazo[1,2-b]pyridazin-2-yl)butyl)-1H-pyrazole-5-carboxamide (23.79 mg, 40.5 μmol, 73% yield), was obtained as a white solid. LCMS [M+H]⁺=572.3 m/z.

### Example 39: Preparation of Compound 23

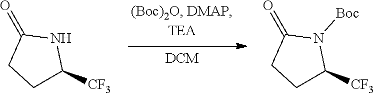 [SMILES: O=C1CC[C@H](C(F)(F)F)N1 ; CC(C)(C)OC(=O)N1C(=O)CC[C@@H]1C(F)(F)F]

To a solution of (R)-5-(trifluoromethyl)pyrrolidin-2-one (5.00 g, 32.7 mmol, 1.00 eq) in DCM (40.0 mL) was added DMAP (1.99 g, 16.3 mmol, 0.500 eq), (Boc)₂O (8.55 g, 39.2 mmol, 9.00 mL, 1.20 eq) and TEA (6.61 g, 65.3 mmol, 9.09 mL, 2.00 eq). The reaction mixture was stirred at RT for 4 h. The crude reaction mixture was diluted with H₂O (15.0 mL) and extracted with DCM (30.0 mL×3), dried over Na₂SO₄, filtered, and concentrated under reduced pressure to give a residue. The residue was purified by column chromatography (SiO₂, EtOAc/PE=3/1). tert-Butyl (R)-2-oxo-5-(trifluoromethyl)pyrrolidine-1-carboxylate was obtained as white solid. ¹H NMR (400 MHz, CDCl₃) δ 4.80-4.68 (s, 1H), 2.80-2.67 (m, 1H), 2.55-2.45 (m, 1H), 2.35-2.15 (m, 2H), 1.53 (s, 9H).

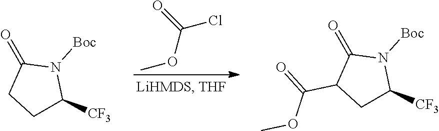

To a solution of tert-butyl (R)-2-oxo-5-(trifluoromethyl)pyrrolidine-1-carboxylate (2.00 g 7.90 mmol, 1.00 eq) in THF (20.0 mL) was added dropwise LiHMDS (1.00 M, 17.4 mL, 2.20 eq) at −70° C. After addition, the reaction mixture was stirred at this temperature for 1 h, and methyl chloroformate (2.20 g, 23.3 mmol, 1.80 mL, 2.95 eq) was added dropwise at −70° C. The resulting mixture was stirred at −70° C. for 1 h. The reaction mixture was diluted with H₂O (15.0 mL) and extracted with EtOAc (20.0 mL×3). The combined organic layers were washed with H₂O (15.0 mL×3), dried over Na₂SO₄, filtered, and concentrated under reduced pressure to give a residue. The residue was purified by column chromatography (SiO₂, PE/EtOAc=3/1). The product, 1-(tert-butyl) 3-methyl (5R)-2-oxo-5-(trifluoromethyl)pyrrolidine-1,3-dicarboxylate (2.20 g, 7.07 mmol, 90% yield), was obtained as yellow oil. ¹H NMR (400 MHz, CDCl₃) δ 4.76-4.73 (m, 1H), 3.86-3.74 (m, 4H), 2.85-2.67 (m, 1H), 2.45-2.35 (m, 1H), 1.53 (s, 9H).

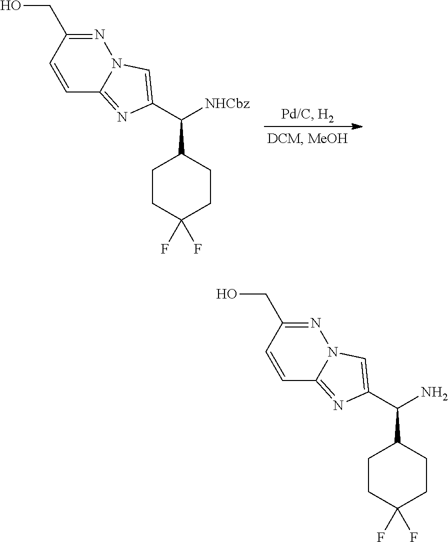 [SMILES: O=C(N[C@H](c1cn2nc(CO)ccc2n1)C1CCC(F)(F)CC1)OCc1ccccc1 ; "Pd/C, H2" ; N[C@H](c1cn2nc(CO)ccc2n1)C1CCC(F)(F)CC1]

To a solution of benzyl (S)-((4,4-difluorocyclohexyl)(6-(hydroxymethyl)imidazo[1,2-b]pyridazin-2-yl)methyl)carbamate (1.00 g, 2.32 mmol, 1.00 eq) in DCM (5.00 mL) and MeOH (0.500 mL) was added Pd/C (250 mg, 10.0% purity) under N₂. The suspension was degassed under vacuum and purged with H₂ several times. The reaction mixture was stirred under H₂ (15 psi) at RT for 20 h. The reaction mixture was filtered with MeOH (150 mL) and the filtrate was concentrated under reduced pressure to give (S)-(2-(amino(4,4-difluorocyclohexyl)methyl)imidazo[1,2-b]pyridazin-6-yl) MeOH (600 mg, 2.02 mmol, 87% yield) as yellow oil. LCMS [M+H]⁺=297.1 m/z.

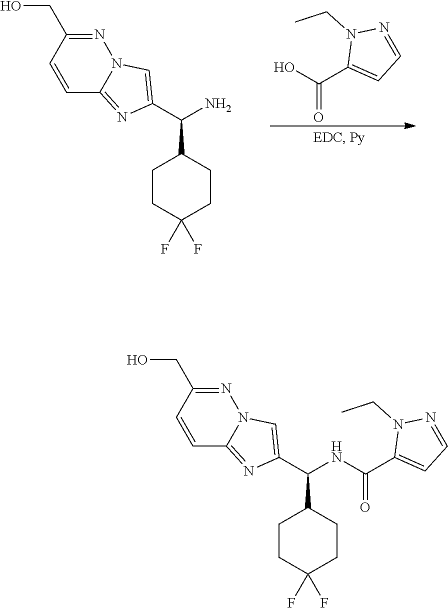

To a solution of (S)-(2-(amino(4,4-difluorocyclohexyl)methyl)imidazo[1,2-b]pyridazin-6-yl) MeOH (500 mg, 1.69 mmol, 1.00 eq) and 1-ethyl-1H-pyrazole-5-carboxylic acid (248 mg, 1.77 mmol, 1.05 eq) in pyridine (5.00 mL) was added EDCI (809 mg, 4.22 mmol, 2.50 eq). The reaction mixture was stirred at RT for 1 h. The residue was diluted with H₂O (15.0 mL) and extracted with EtOAc (15 mL×3). The combined organic layers were dried over Na₂SO₄, filtered, and concentrated under reduced pressure to give a residue. The residue was purified by column chromatography (SiO₂, EtOAc/PE=1/0). The product, (S)—N-((4,4-difluorocyclohexyl)(6-(hydroxymethyl)imidazo[1,2-b]pyridazin-2-yl)methyl)-1-ethyl-1H-pyrazole-5-carboxamide (600 mg, 1.43 mmol, 85% yield), was obtained as a yellow oil. ¹H NMR (400 MHz, CDCl₃) δ 7.90-7.84 (m, 2H), 7.45 (d, J=2.0 Hz, 1H), 7.25-7.13 (m, 2H), 6.63 (d, J=2.0 Hz, 1H), 5.31-5.23 (m, 1H), 4.86 (s, 2H), 4.63-4.55 (m, 2H), 2.17-2.08 (m, 1H), 2.02-1.95 (m, 1H), 1.81-1.61 (m, 5H), 1.57-1.48 (m, 1H), 1.43 (t, J=7.2 Hz, 3H), 1.38-1.31 (m, 1H).

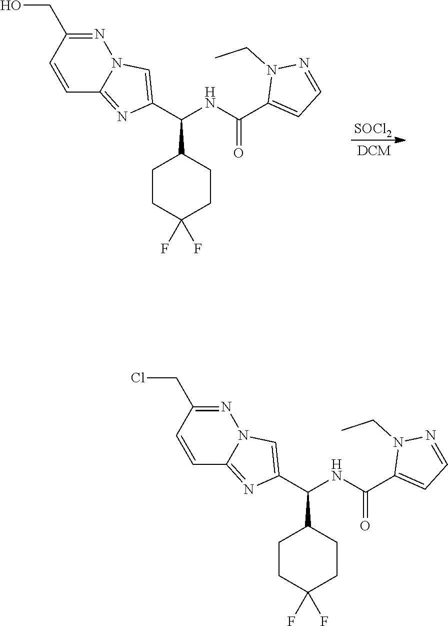

To a solution of (S)—N-((4,4-difluorocyclohexyl)(6-(hydroxymethyl)imidazo[1,2-b]pyridazin-2-yl)methyl)-1-ethyl-1H-pyrazole-5-carboxamide (300 mg, 717 μmol, 1.00 eq) in DCM (4.00 mL) was added SOCl₂ (256 mg, 2.15 mmol, 156 μL, 3.00 eq) at 0° C. The reaction mixture was stirred at 0° C. for 2 h. The reaction mixture was concentrated under reduced pressure to give (S)—N-((6-(chloromethyl)imidazo[1,2-b]pyridazin-2-yl)(4,4-difluorocyclohexyl)methyl)-1-ethyl-1H-pyrazole-5-carboxamide (300 mg, 687 μmol, 96% yield) as yellow solid. LCMS [M+H]⁺=437.2 m/z.

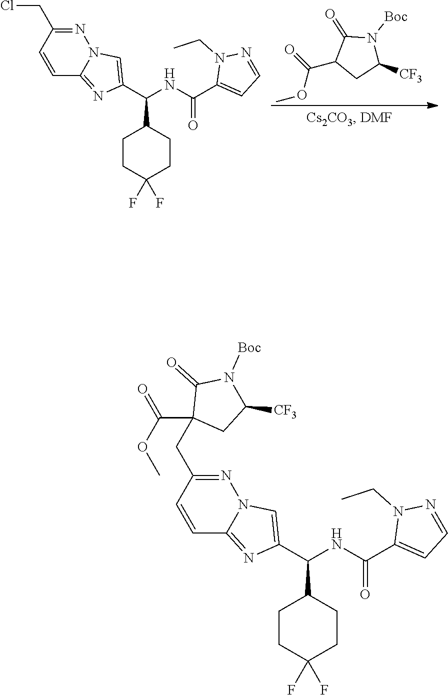

To a solution of (S)—N-((6-(chloromethyl)imidazo[1,2-b]pyridazin-2-yl)(4,4-difluorocyclohexyl)methyl)-1-ethyl-1H-pyrazole-5-carboxamide (160 mg, 366 μmol, 1.00 eq) and 1-(tert-butyl) 3-methyl (5R)-2-oxo-5-(trifluoromethyl)pyrrolidine-1,3-dicarboxylate (171 mg, 549 μmol, 1.50 eq) in DMF (4.00 mL) was added Cs₂CO₃ (477 mg, 1.46 mmol, 4.00 eq). The reaction mixture was stirred at RT for 4 h. The residue was diluted with H₂O (15.0 mL) and extracted with EtOAc (15.0 mL×3). The combined organic layers were dried over Na₂SO₄, filtered, and concentrated under reduced pressure to give a residue. The residue was purified by prep-HPLC (Phenomenex luna C18 150×25 mm×10 μm; mobile phase: [water (FA)-ACN]; B %: 53.0%-83.0%, 10 min). The product, 1-(tert-butyl) 3-methyl (5R)-3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(trifluoromethyl)pyrrolidine-1,3-dicarboxylate (110 mg, 154.6 μmol, 42% yield), was obtained as a yellow solid. LCMS [M+H]⁺=712.3 m/z.

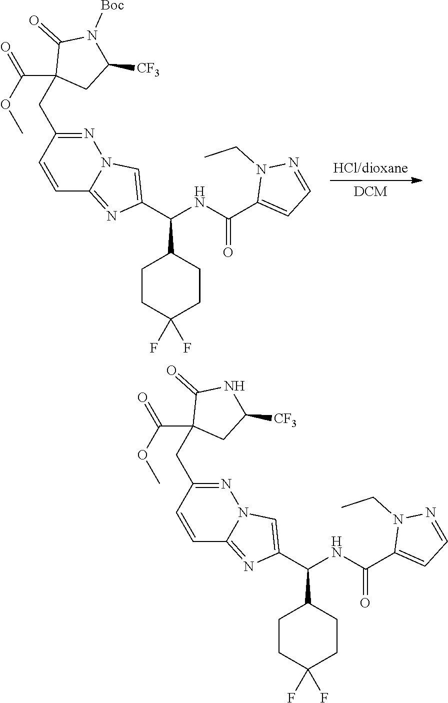

To a solution of 1-(tert-butyl) 3-methyl (5R)-3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(trifluoromethyl)pyrrolidine-1,3-dicarboxylate (90.0 mg, 126.5 μmol, 1.00 eq) in DCM (2.50 mL) was added HCl/dioxane (4.00 M, 2.00 mL, 63.3 eq) at 0° C. The reaction mixture was stirred at RT for 3 h. The reaction mixture was concentrated under reduced pressure to give methyl (5R)-3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(trifluoromethyl)pyrrolidine-3-carboxylate (75.0 mg, 115 μmol, 92% yield, HCl salt) as yellow solid. LCMS [M+H]⁺=612.3 m/z.

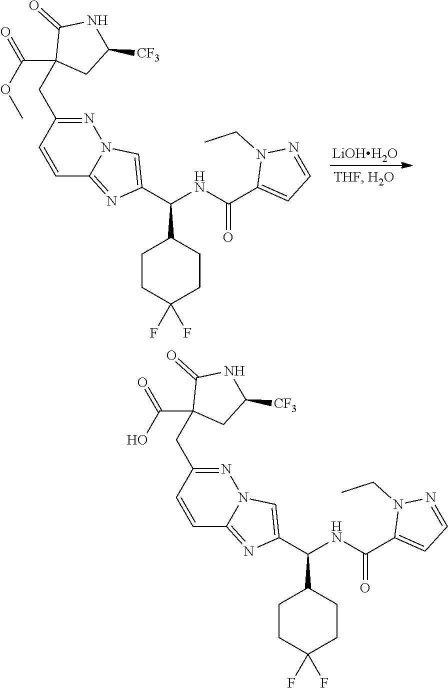

To a solution of methyl (5R)-3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(trifluoromethyl)pyrrolidine-3-carboxylate (75.0 mg, 116 μmol, 1.00 eq, HCl salt) in THF (2.00 mL) was added a solution of LiOH·H₂O (14.6 mg, 347 μmol, 3.00 eq) in H₂O (0.600 mL) at 0° C. The reaction mixture was stirred at RT for 2 h. The reaction mixture was diluted with H₂O (15.0 mL) then concentrated under reduced pressure to remove solvent. The residue was diluted with H₂O (15.0 mL) and adjusted to pH=4 with HCl (1 M), then extracted with EtOAc (30.0 mL). The combined organic layers were dried over Na₂SO₄, filtered, and concentrated under reduced pressure to afford (5R)-3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(trifluoromethyl)pyrrolidine-3-carboxylic acid (68.0 mg, 114 μmol, 98% yield) as yellow solid. LCMS [M+H]⁺=598.3 m/z.

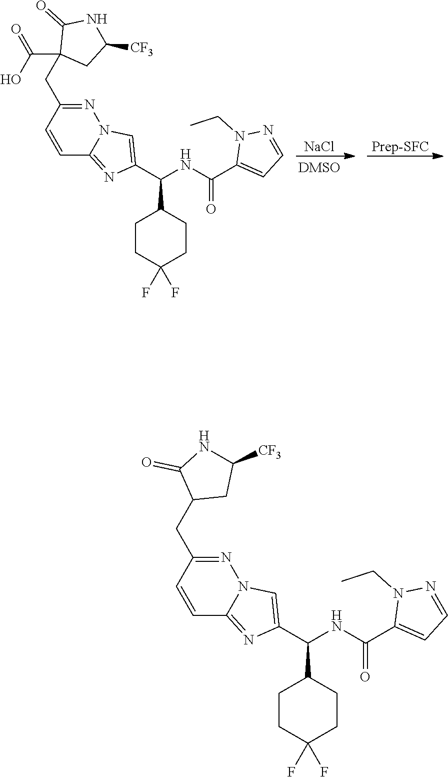

To a solution of (5R)-3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(trifluoromethyl)pyrrolidine-3-carboxylic acid (68.0 mg, 114 μmol, 1.00 eq) in DMSO (1.00 mL) was added NaCl (19.9 mg, 341 μmol, 3.00 eq). The reaction mixture was stirred at 120° C. for 1 h. The residue was diluted with H₂O (15.0 mL) and extracted with EtOAc (15.0 mL×3). Combined organic layers were dried over Na₂SO₄, filtered, and concentrated under reduced pressure to give a residue. The residue was purified by prep-TLC (SiO₂, DCM:MeOH=10:1), to afford crude white solid, N-((1S)-(4,4-difluorocyclohexyl)(6-(((5R)-2-oxo-5-(trifluoromethyl)pyrrolidin-3-yl)methyl)imidazo[1,2-b]pyridazin-2-yl)methyl)-1-ethyl-1H-pyrazole-5-carboxamide (45.0 mg, 81.3 μmol, 71% yield), as a mixture of stereoisomers. The stereoisomeric mixture was separated by SFC (DAICEL CHIRALPAK AS (250 mm×30 mm, 10 μm); 25% mobile phase: [0.1% NH₃H₂O in EtOH]). The first eluting single stereoisomer, Compound 23, N-((1S)-(4,4-difluorocyclohexyl)(6-(((5R)-2-oxo-5-(trifluoromethyl)pyrrolidin-3-yl)methyl)imidazo[1,2-b]pyridazin-2-yl)methyl)-1-ethyl-1H-pyrazole-5-carboxamide (8.94 mg, 15.5 μmol, 22% yield), was obtained as a white solid. LCMS [M+H]⁺=554.3 m/z.

### Example 40: Preparation of Compounds 24 and 25

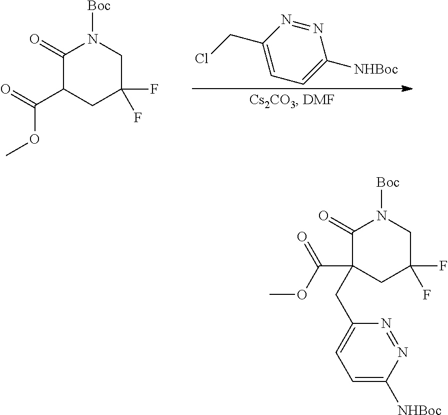 [SMILES: COC(=O)C1CC(F)(F)CN(C(=O)OC(C)(C)C)C1=O ; CC(C)(C)OC(=O)Nc1ccc(CCl)nn1 ; COC(=O)C1(Cc2ccc(NC(=O)OC(C)(C)C)nn2)CC(F)(F)CN(C(=O)OC(C)(C)C)C1=O]

To a solution of tert-butyl (6-(chloromethyl)pyridazin-3-yl)carbamate (1.40 g, 5.75 mmol, 1.00 eq) and 1-(tert-butyl) 3-methyl 5,5-difluoro-2-oxopiperidine-1,3-dicarboxylate (2.02 g, 6.89 mmol, 1.20 eq) in DMF (15.0 mL) was added Cs₂CO₃ (5.62 g, 17.2 mmol, 3.00 eq). The reaction mixture was stirred at 55° C. for 2 h. The reaction mixture was diluted with water (60.0 mL) and extracted with EtOAc (60.0 mL×2). The combined organic layers were washed with brine 30.0 mL, dried over Na₂SO₄, filtered, and concentrated under reduced pressure to give a residue. The residue was purified by column chromatography (SiO₂, PE:EtOAc=10:1 to 3:1). The product, 1-(tert-butyl) 3-methyl 3-((6-((tert-butoxycarbonyl)amino)pyridazin-3-yl)methyl)-5,5-difluoro-2-oxopiperidine-1,3-dicarboxylate (1.20 g, 2.40 mmol, 42% yield), was obtained as a white solid. LCMS [M+H]⁺=501.4 m/z.

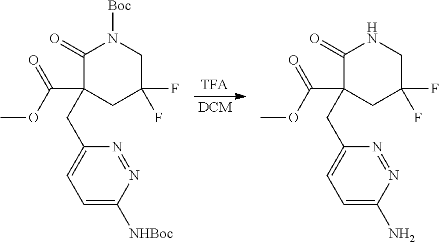 [SMILES: COC(=O)C1(Cc2ccc(NC(=O)OC(C)(C)C)nn2)CC(F)(F)CN(C(=O)OC(C)(C)C)C1=O ; COC(=O)C1(Cc2ccc(N)nn2)CC(F)(F)CNC1=O]

To a solution of 1-(tert-butyl) 3-methyl 3-((6-((tert-butoxycarbonyl)amino)pyridazin-3-yl)methyl)-5,5-difluoro-2-oxopiperidine-1,3-dicarboxylate (1.10 g, 2.20 mmol, 1.00 eq) in DCM (10.0 mL) was added TFA (5.08 g, 44.5 mmol, 3.30 mL, 20.3 eq). The reaction mixture was stirred at RT for 2 h. The reaction mixture was concentrated under reduced pressure to give a residue. The product, methyl 3-((6-aminopyridazin-3-yl)methyl)-5,5-difluoro-2-oxopiperidine-3-carboxylate (600 mg, 1.45 mmol, 66% yield, TFA salt), was obtained as a yellow oil. LCMS [M+H]⁺=300.9 m/z.

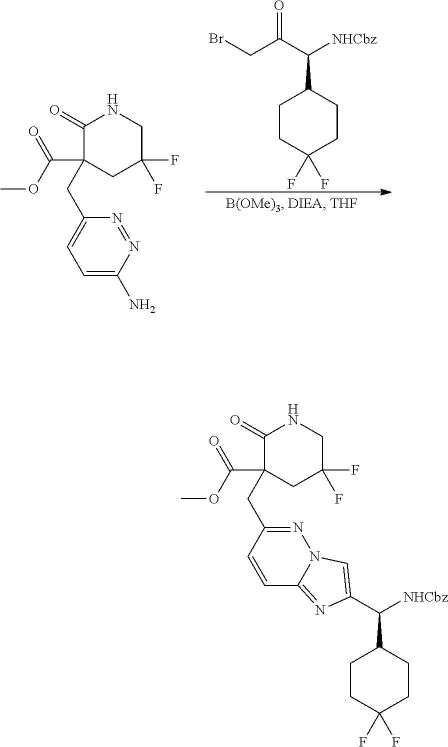 [SMILES: COC(=O)C1(Cc2ccc(N)nn2)CC(F)(F)CNC1=O ; O=C(N[C@H](C(=O)CBr)C1CCC(F)(F)CC1)OCc1ccccc1 ; COC(=O)C1(Cc2ccc3nc([C@@H](NC(=O)OCc4ccccc4)C4CCC(F)(F)CC4)cn3n2)CC(F)(F)CNC1=O]

To a solution of methyl 3-((6-aminopyridazin-3-yl)methyl)-5,5-difluoro-2-oxopiperidine-3-carboxylate (90.0 mg, 217 μmol, 1.00 eq, TFA salt), benzyl (S)-(3-bromo-1-(4,4-difluorocyclohexyl)-2-oxopropyl)carbamate (133 mg, 329 μmol, 1.5 eq) in THF (2.00 mL) was added B(OMe)₃ (186 mg, 1.80 mmol, 203 μL, 8.3 eq), and DIEA (232 mg, 1.80 mmol, 313 μL, 8.3 eq). The reaction mixture was stirred at 70° C. for 2 h. The reaction mixture was diluted with H₂O (30.0 mL) and extracted with EtOAc (30.0 mL×3) and the organic layers were washed with brine (40.0 mL). Then the organic layers were dried over Na₂SO₄, filtered, and concentrated under reduced pressure to give a residue. The residue was purified by prep-TLC (SiO₂, PE:EtOAc=0:1). The product, methyl 3-((2-((S)-(((benzyloxy)carbonyl)amino)(4,4-difluorocyclohexyl)methyl)imidazo[1,2-b]pyridazin-6-yl)methyl)-5,5-difluoro-2-oxopiperidine-3-carboxylate (100 mg, 165 μmol, 55% yield), was obtained as yellow oil. LCMS [M+H]⁺=606.4 m/z.

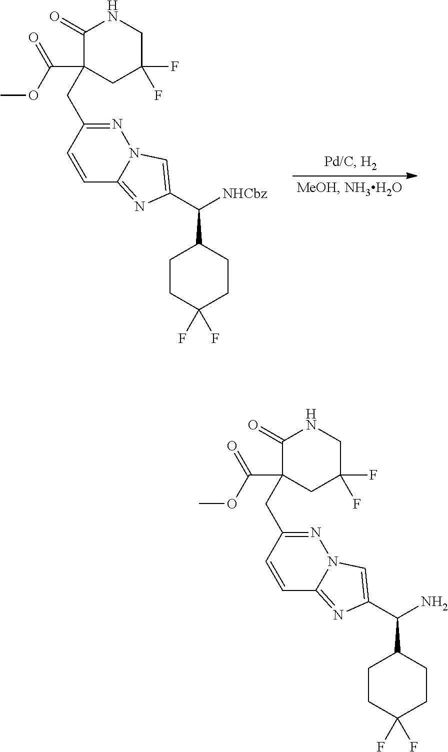 [SMILES: COC(=O)C1(Cc2ccc3nc([C@@H](NC(=O)OCc4ccccc4)C4CCC(F)(F)CC4)cn3n2)CC(F)(F)CNC1=O ; COC(=O)C1(Cc2ccc3nc([C@@H](N)C4CCC(F)(F)CC4)cn3n2)CC(F)(F)CNC1=O]

To a solution of methyl 3-((2-((S)-(((benzyloxy)carbonyl)amino)(4,4-difluorocyclohexyl)methyl)imidazo[1,2-b]pyridazin-6-yl)methyl)-5,5-difluoro-2-oxopiperidine-3-carboxylate (95 mg, 156 μmol, 1.00 eq) in MeOH (3.00 mL) was added NH₃·H₂O (0.300 mL) and Pd/C (30.0 mg, 10.0% purity) under N₂. The suspension was degassed under vacuum and purged with H₂ several times. The reaction mixture was stirred under H₂ (15 psi) at RT for 1 h. The reaction mixture was filtered, and the filtrate was concentrated under reduced pressure to give a residue. The product, methyl 3-((2-((S)-amino(4,4-difluorocyclohexyl)methyl)imidazo[1,2-b]pyridazin-6-yl)methyl)-5,5-difluoro-2-oxopiperidine-3-carboxylate (60.0 mg, 119 μmol, 76% yield), was obtained as a yellow solid. LCMS [M+H]⁺=472.2 m/z.

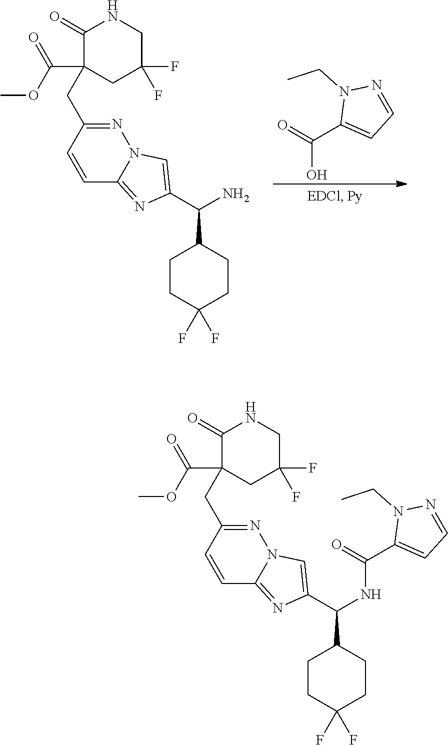

To a solution of methyl 3-((2-((S)-amino(4,4-difluorocyclohexyl)methyl)imidazo[1,2-b]pyridazin-6-yl)methyl)-5,5-difluoro-2-oxopiperidine-3-carboxylate (60.0 mg, 119 μmol, 1.00 eq) and 1-ethyl-1H-pyrazole-5-carboxylic acid (25.2 mg, 179 μmol, 1.50 eq) in pyridine (2.00 mL) was added EDCI (68.9 mg, 359 μmol, 3.00 eq). The reaction mixture was stirred at RT for 1 h. The reaction mixture was diluted with water (10.0 mL) and extracted with DCM (10.0 mL×3). The combined organic layers were dried over Na₂SO₄, filtered, and concentrated under reduced pressure to give a residue. Crude product, methyl 3-((2-((S)-(4,4-difluorocyclohexyl)(1-ethyl-1H-pyrazole-5-carboxamido)methyl)imidazo[1,2-b]pyridazin-6-yl)methyl)-5,5-difluoro-2-oxopiperidine-3-carboxylate (85.0 mg, crude), was obtained as a yellow oil. LCMS [M+H]⁺=594.3 m/z.

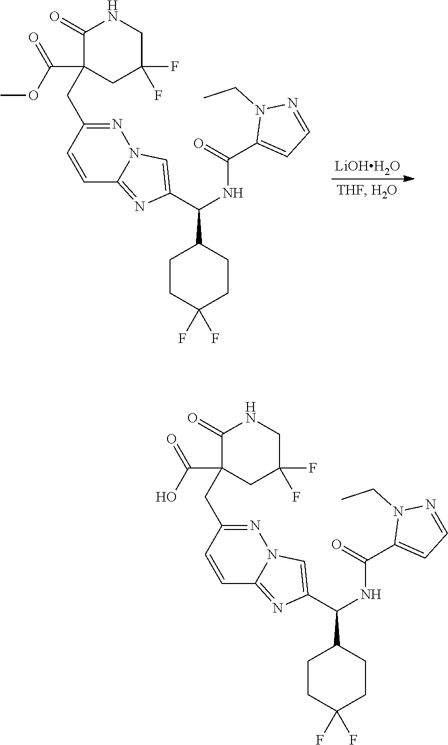

To a solution of methyl 3-((2-((S)-(4,4-difluorocyclohexyl)(1-ethyl-1H-pyrazole-5-carboxamido)methyl)imidazo[1,2-b]pyridazin-6-yl)methyl)-5,5-difluoro-2-oxopiperidine-3-carboxylate (85.0 mg, crude) in THF (3.00 mL) was added a solution of LiOH·H₂O (24.0 mg 572 μmol, 4.00 eq) in H₂O (1.00 mL). The reaction mixture was stirred at RT for 1 h. The reaction mixture was adjusted to pH=3-4 with 1.00 M HCl, and then concentrated under reduced pressure to give a residue. Crude 3-((2-((S)-(4,4-difluorocyclohexyl)(1-ethyl-1H-pyrazole-5-carboxamido)methyl)imidazo[1,2-b]pyridazin-6-yl)methyl)-5,5-difluoro-2-oxopiperidine-3-carboxylic acid (120 mg, crude) was obtained as a yellow oil. LCMS [M+H]⁺=580.2 m/z.

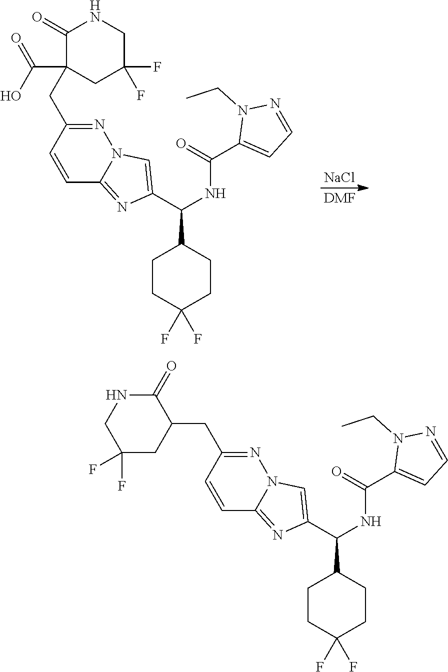

To a solution of 3-((2-((S)-(4,4-difluorocyclohexyl)(1-ethyl-1H-pyrazole-5-carboxamido)methyl)imidazo[1,2-b]pyridazin-6-yl)methyl)-5,5-difluoro-2-oxopiperidine-3-carboxylic acid (120 mg, crude) in DMF (3.00 mL) was added NaCl (36.3 mg, 621 μmol, 3.00 eq). The reaction mixture was stirred at 110° C. for 1 h. The reaction mixture was cooled to RT, and then concentrated under reduced pressure to give a residue. The residue was purified by prep-TLC (SiO₂, DCM:MeOH=10:1, R_{f}=0.50). The stereoisomeric mixture, N-((1S)-(6-((5,5-difluoro-2-oxopiperidin-3-yl)methyl)imidazo[1,2-b]pyridazin-2-yl)(4,4-difluorocyclohexyl)methyl)-1-ethyl-1H-pyrazole-5-carboxamide (40.0 mg, 67.2 μmol, 32% yield over 3 steps), was obtained as a yellow oil. LCMS [M+H]⁺=536.2 m/z. The reaction mixture was purified by prep-SFC (basic condition; DAICEL CHIRALCEL OD (250 mm×30 mm, 10 μm); mobile phase: [0.1% NH₃·H₂O EtOH]; B %: 35%). The first eluting stereoisomer of N-((1S)-(6-((5,5-difluoro-2-oxopiperidin-3-yl)methyl)imidazo[1,2-b]pyridazin-2-yl)(4,4-difluorocyclohexyl)methyl)-1-ethyl-1H-pyrazole-5-carboxamide (Compound 24, 9.91 mg, 18.2 μmol, 54% yield) was obtained as an off-white solid. LCMS [M+H]⁺=536.4 m/z. The second eluting stereoisomer of N-((1S)-(6-((5,5-difluoro-2-oxopiperidin-3-yl)methyl)imidazo[1,2-b]pyridazin-2-yl)(4,4-difluorocyclohexyl)methyl)-1-ethyl-1H-pyrazole-5-carboxamide (Compound 25, 8.85 mg, 14.5 μmol, 43% yield) was also obtained as an off-white solid. LCMS [M+H]⁺=536.4 m/z.

### Example 41: Preparation of Compounds 26 and 27

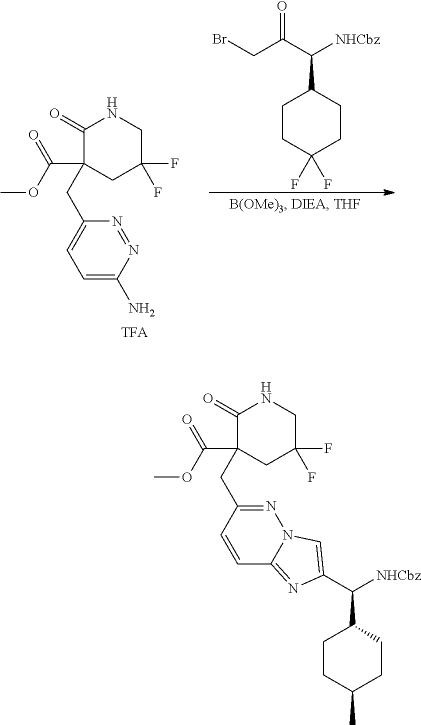 [SMILES: COC(=O)C1(Cc2ccc(N)nn2)CC(F)(F)CNC1=O ; O=C(N[C@H](C(=O)CBr)C1CCC(F)(F)CC1)OCc1ccccc1 ; COC(=O)C1(Cc2ccc3nc([C@@H](NC(=O)OCc4ccccc4)[C@H]4CC[C@H](C)CC4)cn3n2)CC(F)(F)CNC1=O]

To a solution of methyl 3-((6-aminopyridazin-3-yl)methyl)-5,5-difluoro-2-oxopiperidine-3-carboxylate (130 mg, 314 μmol, 1.00 eq, TFA salt), benzyl ((S)-3-bromo-1-((1r,4S)-4-methylcyclohexyl)-2-oxopropyl)carbamate (120 mg, 314 μmol, 1.00 eq) in THF (2.00 mL) was added B(OMe)₃ (196 mg, 1.88 mmol, 213 μL, 6.00 eq), DIEA (243 mg, 1.88 mmol, 328 μL, 6.00 eq), the reaction mixture was stirred at 70° C. for 1.5 h. The reaction mixture was diluted with H₂O (30.0 mL) and extracted with EtOAc (30.0 mL×3). The organic layers were washed with brine (40.0 mL×1), dried over Na₂SO₄, filtered, and concentrated under reduced pressure to give a residue. The residue was used in the next step directly without any purification. Crude product, methyl 3-((2-((S)-(((benzyloxy)carbonyl)amino)((1r,4S)-4-methylcyclohexyl)methyl)imidazo[1,2-b]pyridazin-6-yl)methyl)-5,5-difluoro-2-oxopiperidine-3-carboxylate (170 mg, crude), was obtained as a yellow oil. LCMS [M+H]⁺=584.5 m/z.

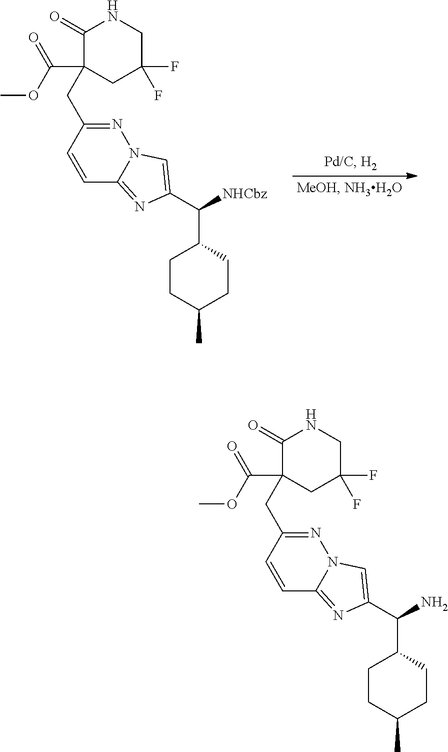 [SMILES: COC(=O)C1(Cc2ccc3nc([C@@H](NC(=O)OCc4ccccc4)[C@H]4CC[C@H](C)CC4)cn3n2)CC(F)(F)CNC1=O ; COC(=O)C1(Cc2ccc3nc([C@@H](N)[C@H]4CC[C@H](C)CC4)cn3n2)CC(F)(F)CNC1=O]

To an N₂-purged flask, Pd/C (60.0 mg, 291 μmol, 10.0% purity) was added followed by a solution of methyl 3-((2-((S)-(((benzyloxy)carbonyl)amino)((1r,4S)-4-methylcyclohexyl)methyl)imidazo[1,2-b]pyridazin-6-yl)methyl)-5,5-difluoro-2-oxopiperidine-3-carboxylate (170 mg, crude) in MeOH (3.00 mL) and NH₃·H₂O (0.300 mL). The suspension was degassed under vacuum and purged with H₂ several times. The reaction mixture was stirred under H₂ (15 psi) at RT for 2 h. The reaction mixture was filtered, and the filtrate was concentrated under reduced pressure. The residue was used in the next step directly without any purification. The product, methyl 3-((2-((S)-amino((1r,4S)-4-methylcyclohexyl)methyl)imidazo[1,2-b]pyridazin-6-yl)methyl)-5,5-difluoro-2-oxopiperidine-3-carboxylate (130 mg, 289 μmol, 92% yield over 2 steps) was obtained as a light-yellow oil. LCMS [M+H]⁺=450.1 m/z.

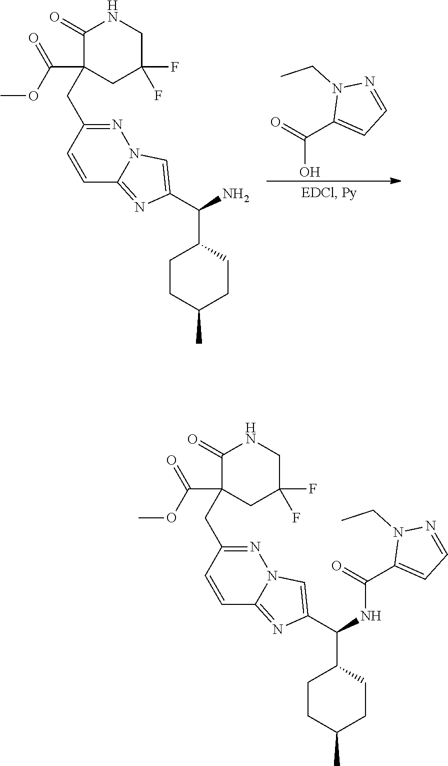

A mixture of methyl 3-((2-((S)-amino((1r,4S)-4-methylcyclohexyl)methyl)imidazo[1,2-b]pyridazin-6-yl)methyl)-5,5-difluoro-2-oxopiperidine-3-carboxylate (130 mg, 289 μmol, 1.00 eq), 1-ethyl-1H-pyrazole-5-carboxylic acid (44.6 mg, 318 μmol, 1.10 eq), and EDCI (166 mg, 868 μmol, 3.00 eq) in pyridine (3.00 mL) was stirred at RT for 2 h. The reaction mixture was diluted with H₂O (30.0 mL) and extracted with EtOAc (30.0 mL×3). The organic layers were washed with brine (40.0 mL) then dried over Na₂SO₄, filtered, and concentrated under reduced pressure to give a residue. The residue was used in the next step directly without any purification. The product, methyl 3-((2-((S)-(1-ethyl-1H-pyrazole-5-carboxamido)((1r,4S)-4-methylcyclohexyl)methyl)imidazo[1,2-b]pyridazin-6-yl)methyl)-5,5-difluoro-2-oxopiperidine-3-carboxylate (150 mg, 262 μmol, 91% yield) was obtained as a yellow oil. LCMS [M+H]⁺=572.2

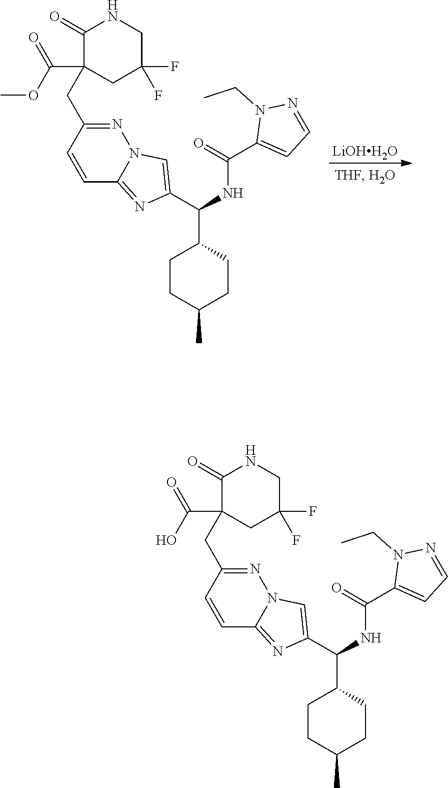

To a solution of methyl 3-((2-((S)-(1-ethyl-1H-pyrazole-5-carboxamido)((1r,4S)-4-methylcyclohexyl)methyl)imidazo[1,2-b]pyridazin-6-yl)methyl)-5,5-difluoro-2-oxopiperidine-3-carboxylate (150 mg, 262 μmol, 1.00 eq) in THF (4.00 mL) and H₂O (1.00 mL) was added LiOH·H₂O (44.1 mg, 1.05 mmol, 4.00 eq). The reaction mixture was stirred at RT for 1 h. The reaction mixture was acidified to pH=5 with HCl (1.00 M) and concentrated under reduced pressure to give a residue. The residue was used in the next step directly without any purification. The product, 3-((2-((S)-(1-ethyl-1H-pyrazole-5-carboxamido)((1r,4S)-4-methylcyclohexyl)methyl)imidazo[1,2-b]pyridazin-6-yl)methyl)-5,5-difluoro-2-oxopiperidine-3-carboxylic acid (140 mg, crude), was obtained as a white solid. LCMS [M+H]⁺=558.2 m/z.

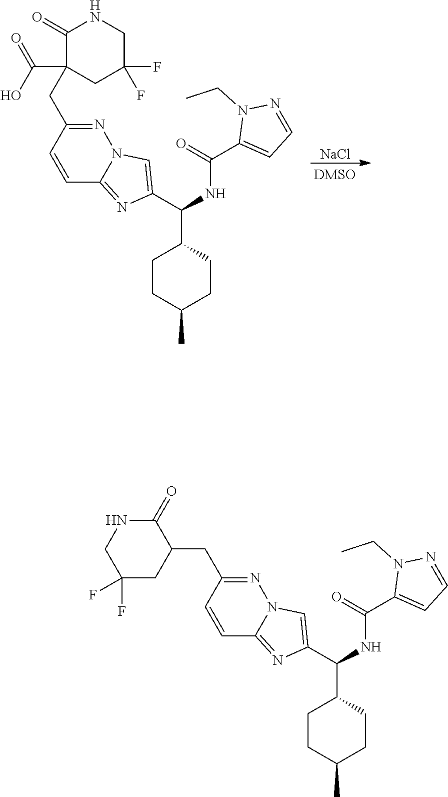

To a solution of 3-((2-((S)-(1-ethyl-1H-pyrazole-5-carboxamido)((1r,4S)-4-methylcyclohexyl)methyl)imidazo[1,2-b]pyridazin-6-yl)methyl)-5,5-difluoro-2-oxopiperidine-3-carboxylic acid (140 mg, crude) in DMF (2.00 mL) was added NaCl (44.0 mg, 753 μmol, 3.00 eq). The reaction mixture was stirred at 110° C. for 1 h. The reaction mixture was diluted with H₂O (30.0 mL) and extracted with EtOAc (30.0 mL×3). The organic layers were washed with brine (40.0 mL×1), dried over Na₂SO₄, filtered, and concentrated under reduced pressure to give a residue. The residue was purified by prep-TLC (SiO₂, DCM:MeOH=10:1). The stereoisomeric mixture, N-((1S)-(6-((5,5-difluoro-2-oxopiperidin-3-yl)methyl)imidazo[1,2-b]pyridazin-2-yl)((1r,4S)-4-methylcyclohexyl)methyl)-1-ethyl-1H-pyrazole-5-carboxamide (90.0 mg, 175 μmol, 67% yield over 2 steps), was obtained as a white solid. LCMS [M+H]⁺=514.2 m/z.

The mixture of stereoisomers was purified by prep-SFC (DAICEL CHIRALCEL OD (250 mm×30 mm, 10 μm); 30% mobile phase: [0.1% NH₃·H₂O EtOH]). The first eluting, single stereoisomer of N-((1S)-(6-((5,5-difluoro-2-oxopiperidin-3-yl)methyl)imidazo[1,2-b]pyridazin-2-yl)((1r,4S)-4-methylcyclohexyl)methyl)-1-ethyl-1H-pyrazole-5-carboxamide (Compound 26, 20.95 mg, 36.9 μmol, 42% yield) was obtained as a white solid. LCMS [M+H]⁺=514.2 m/z. The second eluting, single stereoisomer of N-((1S)-(6-((5,5-difluoro-2-oxopiperidin-3-yl)methyl)imidazo[1,2-b]pyridazin-2-yl)((1r,4S)-4-methylcyclohexyl)methyl)-1-ethyl-1H-pyrazole-5-carboxamide (Compound 27, 22.25 mg, 43.0 μmol, 49% yield) was obtained as a white solid. LCMS [M+H]⁺=514.3 m/z.

### Example 42: Preparation of Compounds 28 and 29

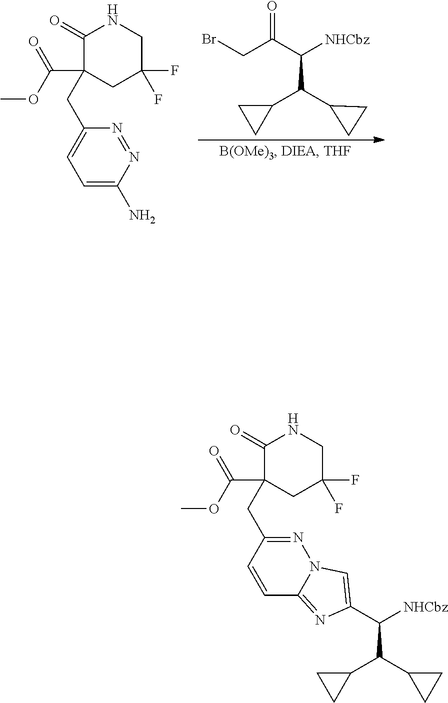 [SMILES: COC(=O)C1(Cc2ccc(N)nn2)CC(F)(F)CNC1=O ; O=C(N[C@H](C(=O)CBr)C(C1CC1)C1CC1)OCc1ccccc1 ; COC(=O)C1(Cc2ccc3nc([C@@H](NC(=O)OCc4ccccc4)C(C4CC4)C4CC4)cn3n2)CC(F)(F)CNC1=O]

To a solution of methyl 3-((6-aminopyridazin-3-yl)methyl)-5,5-difluoro-2-oxopiperidine-3-carboxylate (120 mg, 289 μmol, 1.00 eq, TFA salt) and benzyl (S)-(4-bromo-1,1-dicyclopropyl-3-oxobutan-2-yl)carbamate (121 mg, 318 μmol, 1.10 eq) in THF (2.00 mL) was added trimethyl borate (180 mg, 1.74 mmol, 196 μL, 6.00 eq) and DIEA (224 mg, 1.74 mmol, 302 μL, 6.00 eq). The reaction mixture was stirred at 70° C. for 1.5 h. The reaction mixture was diluted with H₂O (30.0 mL) and extracted with EtOAc (30.0 mL×3). The organic layers were washed with brine (40.0 mL), dried over Na₂SO₄, filtered, and concentrated under reduced pressure to give a residue. The residue was used in the next step directly without any purification. methyl 3-((2-((S)-1-(((benzyloxy)carbonyl)amino)-2,2-dicyclopropylethyl)imidazo[1,2-b]pyridazin-6-yl)methyl)-5,5-difluoro-2-oxopiperidine-3-carboxylate (165 mg, crude) was obtained as a yellow oil. LCMS [M+H]⁺=582.1 m/z.

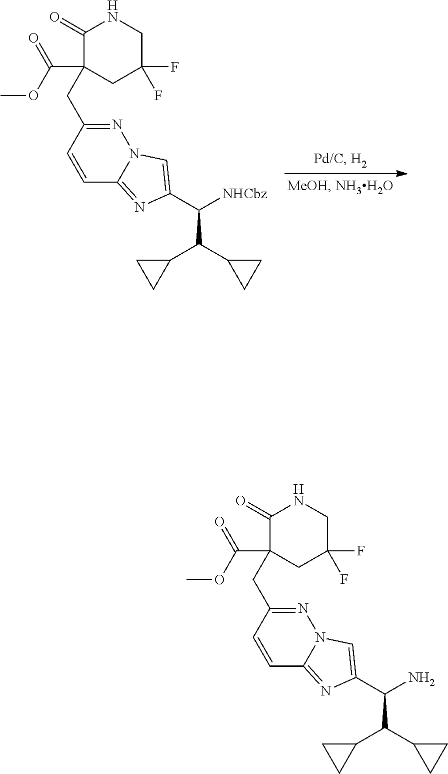 [SMILES: COC(=O)C1(Cc2ccc3nc([C@@H](NC(=O)OCc4ccccc4)C(C4CC4)C4CC4)cn3n2)CC(F)(F)CNC1=O ; COC(=O)C1(Cc2ccc3nc([C@@H](N)C(C4CC4)C4CC4)cn3n2)CC(F)(F)CNC1=O]

To a solution of methyl 3-((2-((S)-1-(((benzyloxy)carbonyl)amino)-2,2-dicyclopropylethyl)imidazo[1,2-b]pyridazin-6-yl)methyl)-5,5-difluoro-2-oxopiperidine-3-carboxylate (155 mg, crude) in MeOH (3.00 mL) and NH₃·H₂O (0.300 mL) was added Pd/C (100 mg, 10.0% purity) under N₂. The suspension was degassed under vacuum and purged with H₂ several times. The reaction mixture was stirred under H₂ (15 psi) at RT for 1 h. The reaction mixture was filtered and the filtrate was concentrated under reduced pressure to give a residue. Crude product, methyl 3-((2-((S)-1-amino-2,2-dicyclopropylethyl)imidazo[1,2-b]pyridazin-6-yl)methyl)-5,5-difluoro-2-oxopiperidine-3-carboxylate (110 mg, crude), was obtained as a yellow oil. LCMS [M+H]⁺=448.3 m/z.

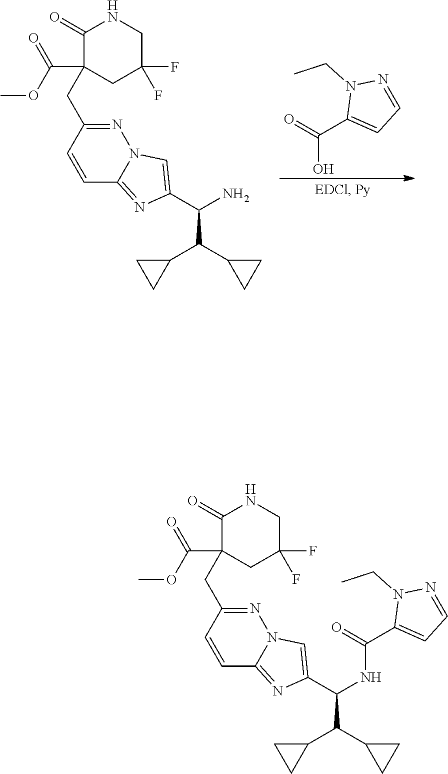

To a solution of methyl 3-((2-((S)-1-amino-2,2-dicyclopropylethyl)imidazo[1,2-b]pyridazin-6-yl)methyl)-5,5-difluoro-2-oxopiperidine-3-carboxylate (110 mg, crude) and 1-ethyl-1H-pyrazole-5-carboxylic acid (51.6 mg, 368 μmol, 1.50 eq) in pyridine (4.00 mL) was added EDCI (141 mg, 737 μmol, 3.00 eq). The reaction mixture was stirred at RT for 1 h. The reaction mixture was diluted with water (10.0 mL) and extracted with DCM (10.0 mL×3). The combined organic layers were dried over Na₂SO₄, filtered, and concentrated under reduced pressure to give a residue. The residue was purified by prep-TLC (SiO₂, PE:EtOAc=0:1, R_{f}=0.10). Methyl 3-((2-((S)-2,2-dicyclopropyl-1-(1-ethyl-1H-pyrazole-5-carboxamido)ethyl)imidazo[1,2-b]pyridazin-6-yl)methyl)-5,5-difluoro-2-oxopiperidine-3-carboxylate (95.0 mg, 165 μmol, 57% yield over 3 steps) was obtained as a yellow oil. LCMS [M+H]⁺=570.4 m/z.

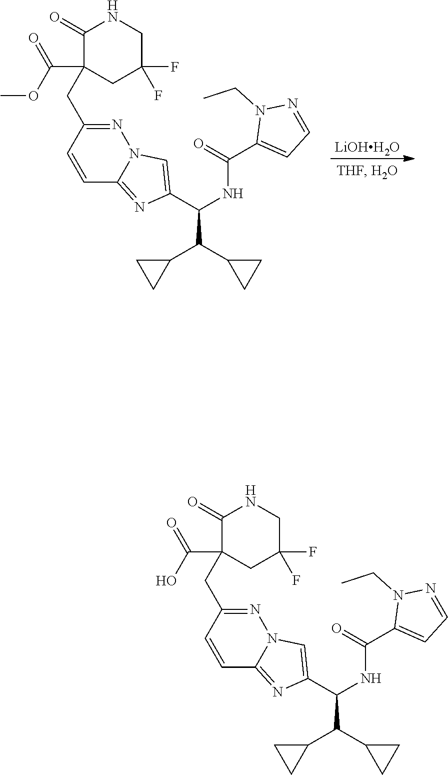

To a solution of methyl 3-((2-((S)-2,2-dicyclopropyl-1-(1-ethyl-1H-pyrazole-5-carboxamido)ethyl)imidazo[1,2-b]pyridazin-6-yl)methyl)-5,5-difluoro-2-oxopiperidine-3-carboxylate (95.0 mg, 165 μmol, 1.00 eq) in THF (3.00 mL) was added a solution of LiOH·H₂O (27.8 mg, 662 μmol, 4.00 eq) in H₂O (1.00 mL). The reaction mixture was stirred at RT for 1 h. The reaction mixture was adjusted to pH=3-4 with 1 M HCl then concentrated under reduced pressure to give a residue. The crude product, 3-((2-((S)-2,2-dicyclopropyl-1-(1-ethyl-1H-pyrazole-5-carboxamido)ethyl)imidazo[1,2-b]pyridazin-6-yl)methyl)-5,5-difluoro-2-oxopiperidine-3-carboxylic acid (100 mg, crude), was obtained as a yellow solid. LCMS [M+H]⁺=556.2 m/z.

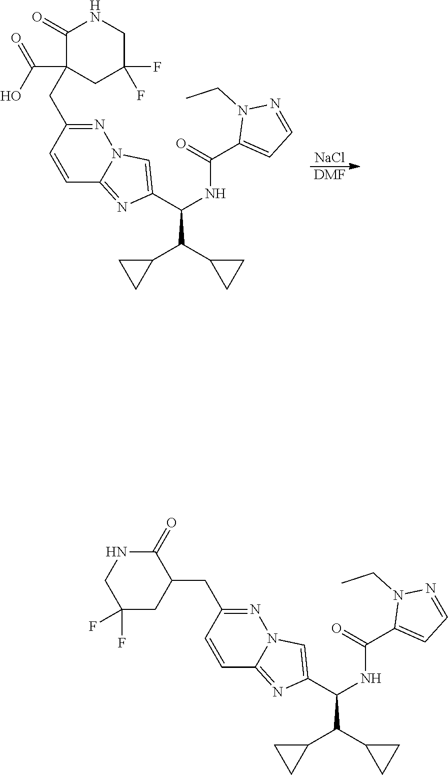

To a solution 3-((2-((S)-2,2-dicyclopropyl-1-(1-ethyl-1H-pyrazole-5-carboxamido)ethyl)imidazo[1,2-b]pyridazin-6-yl)methyl)-5,5-difluoro-2-oxopiperidine-3-carboxylic acid (100 mg, crude) in DMF (2.00 mL) was added NaCl (31.5 mg, 539 μmol, 3.00 eq). The reaction mixture was stirred at 110° C. for 1 h. The reaction mixture was cooled to RT, and then concentrated under reduced pressure to give a residue. The residue was purified by prep-TLC (SiO₂, DCM:MeOH=10:1, R_{f}=0.50). The mixture of stereoisomers, N-((1S)-2,2-dicyclopropyl-1-(6-((5,5-difluoro-2-oxopiperidin-3-yl)methyl)imidazo[1,2-b]pyridazin-2-yl)ethyl)-1-ethyl-1H-pyrazole-5-carboxamide (50.0 mg, 97.7 μmol, 59% yield over 2 steps), was obtained as a yellow oil. LCMS [M+H]⁺=512.5 m/z. The mixture of stereoisomers was purified by prep-SFC (basic condition; DAICEL CHIRALPAK AS (250 mm×30 mm, 10 μm); 35% mobile phase: [0.1% NH₃H₂O MeOH]). The first eluting stereoisomer of N-((1S)-2,2-dicyclopropyl-1-(6-((5,5-difluoro-2-oxopiperidin-3-yl)methyl)imidazo[1,2-b]pyridazin-2-yl)ethyl)-1-ethyl-1H-pyrazole-5-carboxamide (Compound 28, 15.68 mg, 30.6 μmol, 63% yield) was obtained as an off-white solid. LCMS [M+H]⁺=512.5 m/z. The second eluting stereoisomer of N-((1S)-2,2-dicyclopropyl-1-(6-((5,5-difluoro-2-oxopiperidin-3-yl)methyl)imidazo[1,2-b]pyridazin-2-yl)ethyl)-1-ethyl-1H-pyrazole-5-carboxamide (Compound 29, 16.39 mg, 31.8 μmol, 65% yield) was obtained as an off-white solid. LCMS [M+H]⁺=512.5 m/z.

### Example 43: Preparation of Compounds 30 and 31

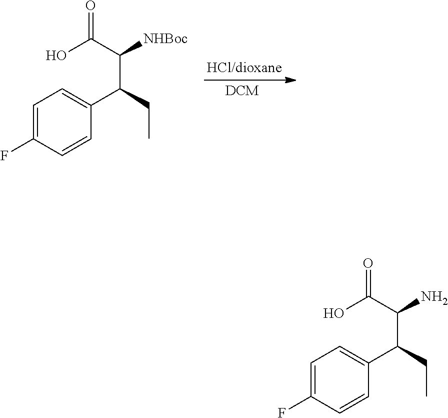 [SMILES: CC[C@@H](c1ccc(F)cc1)[C@H](NC(=O)OC(C)(C)C)C(=O)O ; CC[C@@H](c1ccc(F)cc1)[C@H](N)C(=O)O]

To a solution of (2S,3S)-2-((tert-butoxycarbonyl)amino)-3-(4-fluorophenyl)pentanoic acid (4.80 g, 15.4 mmol, 1.00 eq) in DCM (50.0 mL) was added HCl/dioxane (4.00 M, 20.0 mL, 5.19 eq). The reaction mixture was stirred at RT for 1 h. The reaction mixture was concentrated under reduced pressure to give a residue. (2S,3S)-2-amino-3-(4-fluorophenyl)pentanoic acid (3.50 g, crude HCl salt) was obtained as a yellow solid. LCMS [M+H]⁺=212.1 m/z.

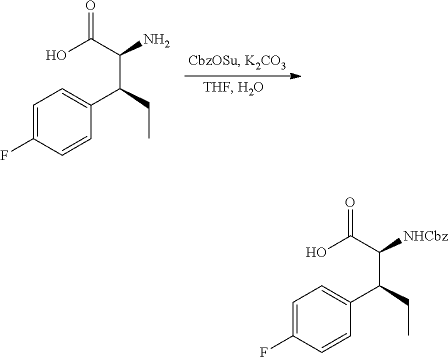 [SMILES: CC[C@@H](c1ccc(F)cc1)[C@H](N)C(=O)O ; CC[C@@H](c1ccc(F)cc1)[C@H](NC(=O)OCc1ccccc1)C(=O)O]

To a solution of (2S,3S)-2-amino-3-(4-fluorophenyl)pentanoic acid (3.50 g, crude HCl salt) in THF (30.0 mL) and H₂O (30.0 mL) was added CbzOSu (4.13 g, 16.5 mmol, 1.00 eq) and K₂CO₃ (6.87 g, 49.7 mmol, 3.00 eq). The reaction mixture was stirred at RT for 12 h. The reaction mixture was acidified to pH=5 with HCl (1.00 M) and extracted with DCM (60.0 mL×3). The organic layers were washed brine (60.0 mL×2) and concentrated under reduced pressure to give a residue. The residue was purified by column chromatography (SiO₂, PE:EtOAc=1:0 to 1:1). The product, (2S,3S)-2-(((benzyloxy)carbonyl)amino)-3-(4-fluorophenyl)pentanoic acid (5.00 g 14.4 mmol, 94% yield over 2 steps), was obtained as a white solid. LCMS [M+Na]⁺=368.1 m/z. ¹H NMR (400 MHz, CDCl₃) δ 12.79 (s, 1H), 7.38-7.23 (m, 6H), 7.18-7.16 (m, 1H), 7.09 (t, J=9.2 Hz, 2H), 5.03-4.87 (m, 2H), 4.24 (t, J=9.2 Hz, 1H), 2.91-2.85 (m, 1H), 1.71-1.53 (m, 2H), 0.65 (t, J=7.2 Hz, 3H).

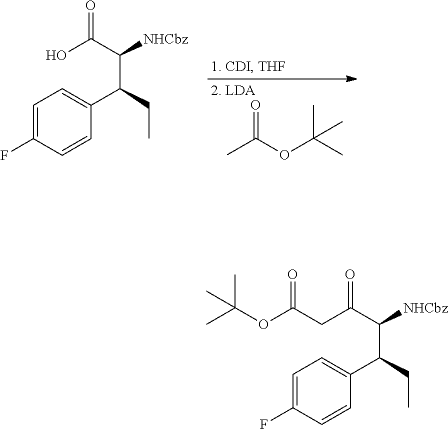

To a solution of (2S,3S)-2-(((benzyloxy)carbonyl)amino)-3-(4-fluorophenyl)pentanoic acid (2.00 g, 5.79 mmol, 1.00 eq) in THF (20.0 mL) was added CDI (1.13 g, 6.95 mmol, 1.20 eq) at 0° C. and the resulting mixture was stirred at 0° C. for 2 h. Meanwhile, to a solution of tert-butyl acetate (2.02 g, 17.4 mmol, 2.33 mL, 3.00 eq) in THF (20.0 mL) was added LDA (2.00 M, 8.69 mL, 3.00 eq) at −78° C. and the reaction mixture was stirred at −78° C. for 2 h. Then the two reaction solutions were mixed and stirred at −78° C. for 2 h. The residue was diluted saturated aqueous NH₄Cl (40.0 mL) and extracted with EtOAc (60.0 mL×3), dried over Na₂SO₄, filtered, and concentrated under reduced pressure to give a residue. The residue was purified by column chromatography (SiO₂, PE:EtOAc=1:0 to 5:1). The product, tert-butyl (4S,5S)-4-(((benzyloxy)carbonyl)amino)-5-(4-fluorophenyl)-3-oxoheptanoate (1.60 g, 3.61 mmol, 62% yield), was obtained as a yellow oil. LCMS [M+Na]⁺=466.3 m/z.

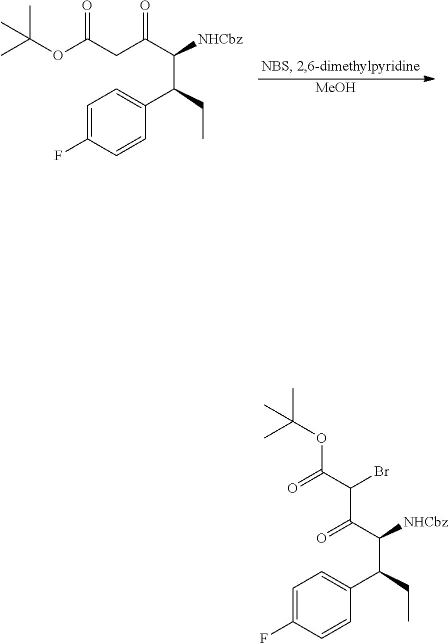 [SMILES: "MeOH" ; CC[C@@H](c1ccc(F)cc1)[C@H](NC(=O)OCc1ccccc1)C(=O)CC(=O)OC(C)(C)C ; CC[C@@H](c1ccc(F)cc1)[C@H](NC(=O)OCc1ccccc1)C(=O)C(Br)C(=O)OC(C)(C)C]

To a solution of tert-butyl (4S,5S)-4-(((benzyloxy)carbonyl)amino)-5-(4-fluorophenyl)-3-oxoheptanoate (1.60 g, 3.61 mmol, 1.00 eq) in MeOH (16.0 mL) was added NBS (513 mg, 2.89 mmol, 0.800 eq) and 2,6-dimethylpyridine (30.9 mg, 288 μmol, 33.6 μL, 0.0800 eq). The reaction mixture was stirred at RT for 1 h. The reaction mixture was concentrated under reduced pressure to give a residue. Crude tert-butyl (4S,5S)-4-(((benzyloxy)carbonyl)amino)-2-bromo-5-(4-fluorophenyl)-3-oxoheptanoate (1.80 g, crude) was obtained as a yellow oil. LCMS [M+H₃O]⁺=540.9 m/z.

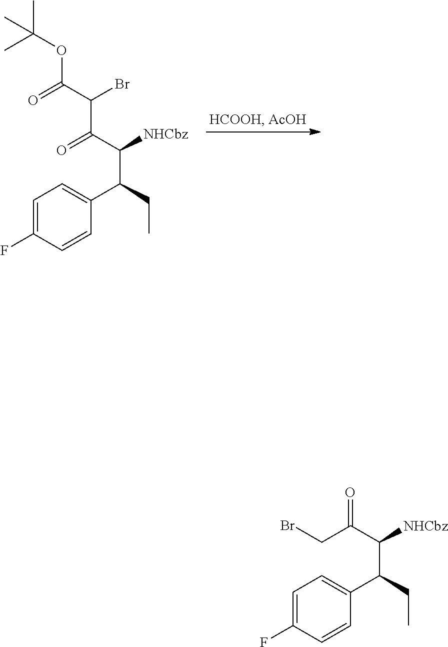 [SMILES: CC[C@@H](c1ccc(F)cc1)[C@H](NC(=O)OCc1ccccc1)C(=O)C(Br)C(=O)OC(C)(C)C ; CC[C@@H](c1ccc(F)cc1)[C@H](NC(=O)OCc1ccccc1)C(=O)CBr]

A solution of tert-butyl (4S,5S)-4-(((benzyloxy)carbonyl)amino)-2-bromo-5-(4-fluorophenyl)-3-oxoheptanoate (1.80 g) in HCOOH (165 mg, 3.45 mmol, 5.00 mL, 1.00 eq) and AcOH (9.45 g, 157 mmol, 9.00 mL, 45.6 eq) was stirred at 80° C. for 1 h. The residue was diluted with saturated aqueous NaHCO₃ to pH=7 and extracted with EtOAc (80.0 mL×3), dried over Na₂SO₄, filtered, and concentrated under reduced pressure to give a residue. The residue was purified by column chromatography (SiO₂, PE:EtOAc=1:0 to 5:1). Benzyl ((4S)-1-bromo-4-(4-fluorophenyl)-2-oxohexan-3-yl)carbamate (1.00 g, 2.37 mmol, 66% yield over 2 steps) was obtained as a yellow oil. SFC shows 55/45 mixture of stereoisomers. LCMS [M+H]⁺=422.1 m/z. ¹H NMR (400 MHz, CDCl₃) δ 8.03 (d, J=8.4 Hz, 0.5H), 7.63 (d, J=8.8 Hz, 0.5H), 7.38-7.22 (m, 6H), 7.16-7.07 (m, 3H), 5.10-4.85 (m, 2H), 4.63-4.47 (m, 2H), 4.45-4.24 (m, 1H), 3.01-2.95 (m, 1H), 1.73-1.49 (m, 2H), 0.61 (q, J=7.2 Hz, 3H).

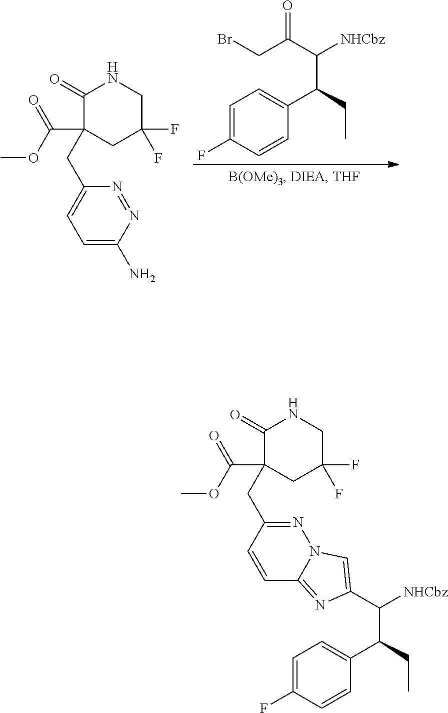

To a solution of methyl 3-((6-aminopyridazin-3-yl)methyl)-5,5-difluoro-2-oxopiperidine-3-carboxylate (200 mg, 482 μmol, 1.00 eq, TFA salt) and benzyl ((4S)-1-bromo-4-(4-fluorophenyl)-2-oxohexan-3-yl)carbamate (244 mg, 579 μmol, 1.20 eq) in THE (3.00 mL) was added B(OMe)₃ (301 mg, 2.90 mmol, 327 μL, 6.00 eq) and DIEA (374 mg, 2.90 mmol, 504 μL, 6.00 eq). The reaction mixture was stirred at 70° C. for 2 h. The reaction mixture was diluted with water (40.0 mL) and extracted with EtOAc (40.0 mL×2). The combined organic layers were washed with brine (40.0 mL), dried over Na₂SO₄, filtered, and concentrated under reduced pressure to give a residue. The residue was purified by prep-TLC (SiO₂, PE:EtOAc=0:1, R_{f}=0.39). Methyl 3-((2-((2S)-1-(((benzyloxy)carbonyl)amino)-2-(4-fluorophenyl)butyl)imidazo[1,2-b]pyridazin-6-yl)methyl)-5,5-difluoro-2-oxopiperidine-3-carboxylate (200 mg, 320 μmol, 66% yield) was obtained as a yellow oil. LCMS [M+H]⁺=624.4 m/z.

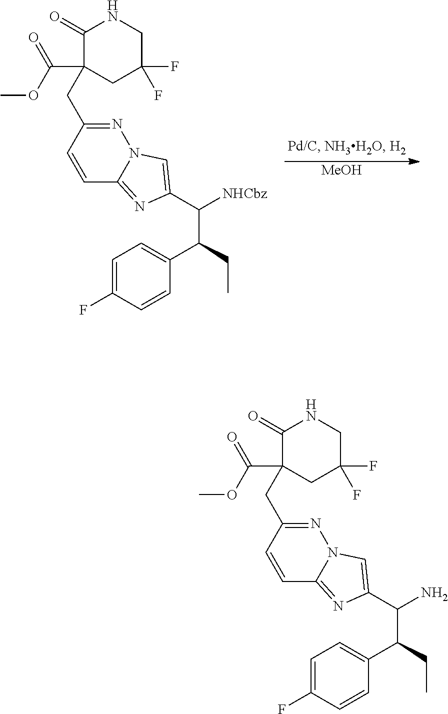 [SMILES: "MeOH" ; CC[C@@H](c1ccc(F)cc1)C(NC(=O)OCc1ccccc1)c1cn2nc(CC3(C(=O)OC)CC(F)(F)CNC3=O)ccc2n1 ; CC[C@@H](c1ccc(F)cc1)C(N)c1cn2nc(CC3(C(=O)OC)CC(F)(F)CNC3=O)ccc2n1]

To a solution of methyl 3-((2-((2S)-1-(((benzyloxy)carbonyl)amino)-2-(4-fluorophenyl)butyl)imidazo[1,2-b]pyridazin-6-yl)methyl)-5,5-difluoro-2-oxopiperidine-3-carboxylate (200 mg, 320 μmol, 1.00 eq) in MeOH (4.00 mL) was added Pd/C (20.0 mg, 10.0% purity) and NH₃·H₂O (182 mg, 1.30 mmol, 0.200 mL, 25.0% purity, 4.05 eq), and then the reaction mixture was stirred at RT for 1 h under H₂ (15 psi) atmosphere. The reaction mixture was filtered, and the filtrate was concentrated under reduced pressure. Methyl 3-((2-((2S)-1-amino-2-(4-fluorophenyl)butyl)imidazo[1,2-b]pyridazin-6-yl)methyl)-5,5-difluoro-2-oxopiperidine-3-carboxylate (150 mg, 306 μmol, 96% yield) was obtained as a yellow solid. LCMS [M+H]⁺=490.3 m/z.

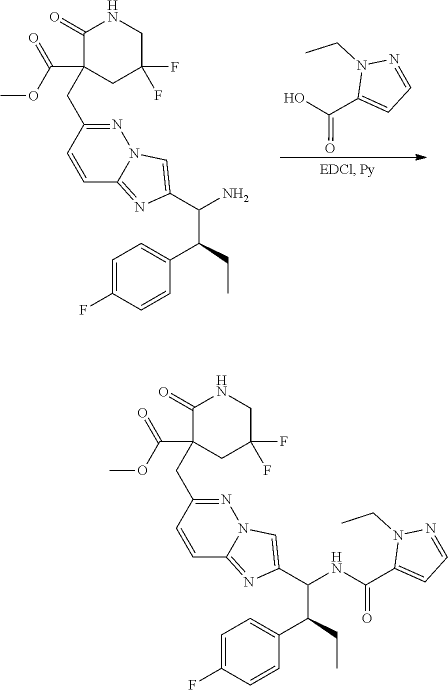

To a solution of methyl 3-((2-((2S)-1-amino-2-(4-fluorophenyl)butyl)imidazo[1,2-b]pyridazin-6-yl)methyl)-5,5-difluoro-2-oxopiperidine-3-carboxylate (150 mg, 306 μmol, 1.00 eq) and 1-ethyl-1H-pyrazole-5-carboxylic acid (64.4 mg, 459 μmol, 1.50 eq) in pyridine (3.00 mL) was added EDCI (235 mg, 1.23 mmol, 4.00 eq). The reaction mixture was stirred at RT for 2 h. The reaction mixture was diluted with H₂O (20.0 mL) and extracted with DCM (20.0 mL×3). The combined organic phase was dried over Na₂SO₄ and concentrated to give a residue. The residue was purified by prep-TLC (SiO₂, PE:EtOAc=0:1, R_{f}=0.36). The product, methyl 3-((2-((2S)-1-(1-ethyl-1H-pyrazole-5-carboxamido)-2-(4-fluorophenyl)butyl)imidazo[1,2-b]pyridazin-6-yl)methyl)-5,5-difluoro-2-oxopiperidine-3-carboxylate (170 mg, 278 μmol, 91% yield), was obtained as a yellow oil. SFC shows 37/35/15/13 mixture of stereoisomers. LCMS [M+H]⁺=612.7 m/z.

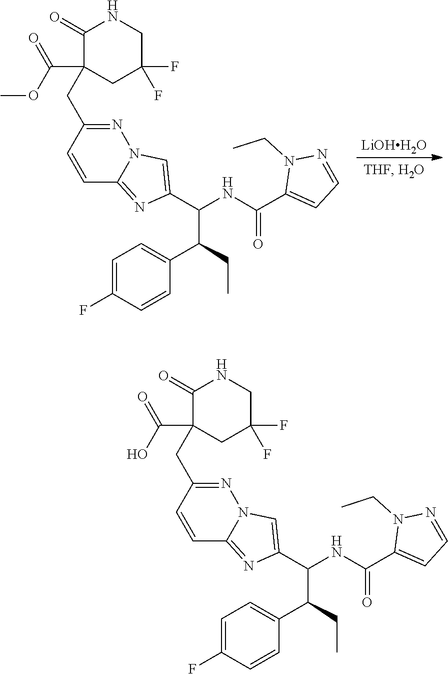

To a solution of methyl 3-((2-((2S)-1-(1-ethyl-1H-pyrazole-5-carboxamido)-2-(4-fluorophenyl)butyl)imidazo[1,2-b]pyridazin-6-yl)methyl)-5,5-difluoro-2-oxopiperidine-3-carboxylate (160 mg, 261 μmol, 1.00 eq) in THF (3.00 mL) and H₂O (1.00 mL) was added LiOH·H₂O (54.9 mg, 1.31 mmol, 5.00 eq). The reaction mixture was stirred at RT for 30 min. The reaction mixture was acidified to pH=5 with HCl (1.00 M) and concentrated under reduced pressure to give a residue. The crude product, 3-((2-((2S)-1-(1-ethyl-1H-pyrazole-5-carboxamido)-2-(4-fluorophenyl)butyl)imidazo[1,2-b]pyridazin-6-yl)methyl)-5,5-difluoro-2-oxopiperidine-3-carboxylic acid (180 mg, crude), was obtained as a yellow oil. LCMS [M+H]⁺=598.4 m/z.

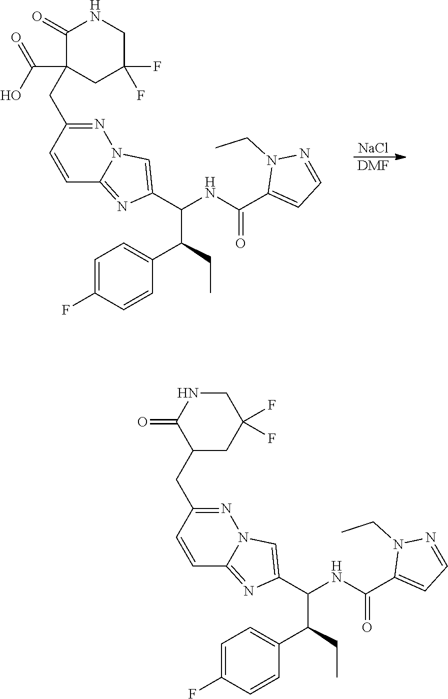

To a solution of 3-((2-((2S)-1-(1-ethyl-1H-pyrazole-5-carboxamido)-2-(4-fluorophenyl)butyl)imidazo[1,2-b]pyridazin-6-yl)methyl)-5,5-difluoro-2-oxopiperidine-3-carboxylic acid (170 mg) in DMF (2.00 mL) was added NaCl (49.8 mg, 853 μmol, 3.00 eq). The reaction mixture was stirred at 110° C. for 30 min. The reaction mixture was concentrated under reduced pressure to give a residue. The residue was purified by prep-TLC (SiO₂, DCM:MeOH=10:1, R_{f}=0.43). As a mixture of 4 stereoisomers, N-((2S)-1-(6-((5,5-difluoro-2-oxopiperidin-3-yl)methyl)imidazo[1,2-b]pyridazin-2-yl)-2-(4-fluorophenyl)butyl)-1-ethyl-1H-pyrazole-5-carboxamide (110 mg, 198 μmol, 76% yield over 2 steps) was obtained as a white solid. SFC shows 37/34/16/13 ratio of stereoisomers. LCMS [M+H]⁺=554.2 m/z.

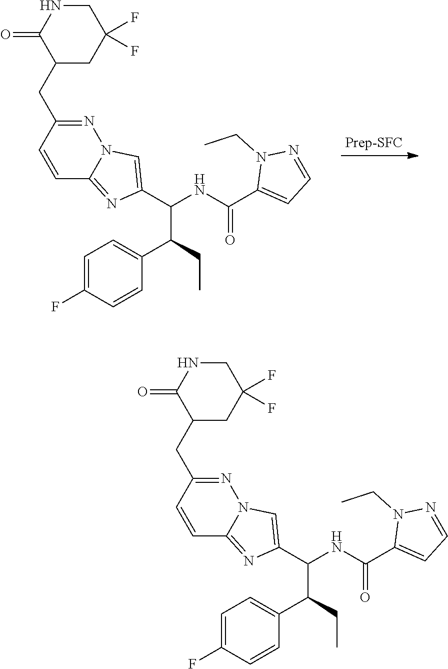 [SMILES: CC[C@@H](c1ccc(F)cc1)C(NC(=O)c1ccnn1CC)c1cn2nc(CC3CC(F)(F)CNC3=O)ccc2n1 ; CC[C@@H](c1ccc(F)cc1)C(NC(=O)c1ccnn1CC)c1cn2nc(CC3CC(F)(F)CNC3=O)ccc2n1]

The two major stereoisomers of N-((2S)-1-(6-((5,5-difluoro-2-oxopiperidin-3-yl)methyl)imidazo[1,2-b]pyridazin-2-yl)-2-(4-fluorophenyl)butyl)-1-ethyl-1H-pyrazole-5-carboxamide were separated by prep-SFC. First purification by prep-SFC (REGIS (S, S) WHELK-O1 (250 mm×25 mm, 10 μm); mobile phase: [0.1% NH₃·H₂O MeOH]; B %: 40%-40%, C8.7; 61 min). Second purification by prep-SFC (DAICEL CHIRALPAK AS (250 mm×30 mm, 10 μm); mobile phase: [0.1% NH₃·H₂O MeOH]; B %: 20%-20%, C8.3; 83 min). Third purification by prep-SFC (DAICEL CHIRALPAK AS (250 mm×30 mm, 10 μm); mobile phase: [0.1% NH₃H₂O MeOH]; B %: 25%-25%, 4.5 min, Rt=1.726, 1.428 min). The first major stereoisomer of N-((2S)-1-(6-((5,5-difluoro-2-oxopiperidin-3-yl)methyl)imidazo[1,2-b]pyridazin-2-yl)-2-(4-fluorophenyl)butyl)-1-ethyl-1H-pyrazole-5-carboxamide (Compound 30, 13.5 mg, 23.0 μmol, 39% yield) by SFC analysis: Rt=1.234 min, (DAICEL CHIRALPAK AS-3 (50×4.6 mm, 3 um); mobile phase: [0.05% DEA·MeOH]; B %: 5%-40%, 3 mL/min, 35° C.; Back Pressure: 100 bar. LCMS [M+H]⁺=554.4 m/z. The second major stereoisomer of N-((2S)-1-(6-((5,5-difluoro-2-oxopiperidin-3-yl)methyl)imidazo[1,2-b]pyridazin-2-yl)-2-(4-fluorophenyl)butyl)-1-ethyl-1H-pyrazole-5-carboxamide (Compound 31, 12.3 mg, 21.5 μmol, 36% yield) by SFC analysis: Rt=1.497 min, (DAICEL CHIRALPAK AS-3 (50×4.6 mm, 3 um); mobile phase: [0.05% DEA·EtOH]; B %: 5%-40%, 3 mL/min, 35° C.; Back Pressure: 100 bar. LCMS [M+H]⁺=554.4 m/z.

### Example 44: Preparation of Compound 32

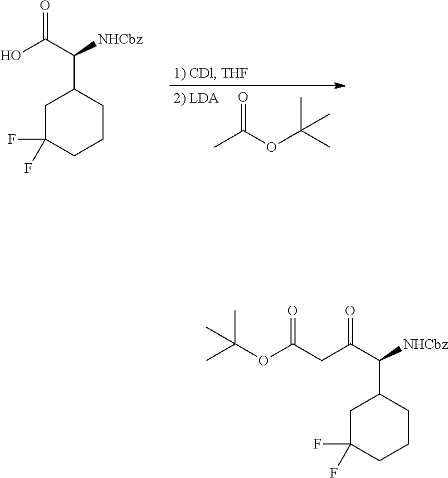

To a solution of (2S)-2-(((benzyloxy)carbonyl)amino)-2-(3,3-difluorocyclohexyl)acetic acid (2.00 g, 6.11 mmol, 1.00 eq, SFC 1/1) in THF (20.0 mL) was added CDI (1.09 g, 6.72 mmol, 1.10 eq) at 0° C. and the resulting mixture was stirred at 0° C. for 2 h. Meanwhile, to a solution of tert-butyl acetate (2.13 g, 18.3 mmol, 2.46 mL, 3.00 eq) in THE (20.0 mL) was added LDA (2.00 M, 9.17 mL, 3.00 eq) at −78° C. and the reaction mixture was stirred at −78° C. for 2 h. The two reaction mixtures were combined and stirred at −78° C. for 2 h. The residue was diluted with saturated aqueous NH₄Cl (40.0 mL) and extracted with EtOAc (60.0 mL×3). The combined organic layers were dried over Na₂SO₄, filtered, and concentrated under reduced pressure to give a residue. The residue was purified by column chromatography (SiO₂, PE:EtOAc=1:0 to 10:1). The product, tert-butyl (4S)-4-(((benzyloxy)carbonyl)amino)-4-(3,3-difluorocyclohexyl)-3-oxobutanoate (1.50 g, 3.53 mmol, 58% yield), was obtained as a yellow oil. LCMS [M−99]⁺=325.9 m/z.

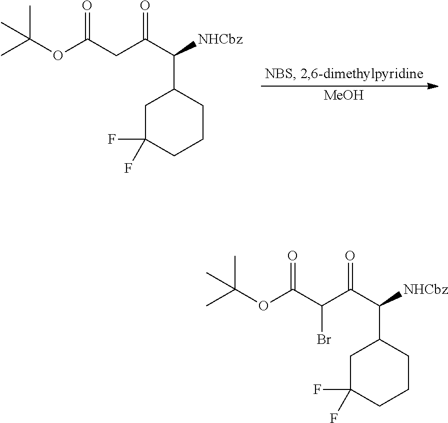 [SMILES: "MeOH" ; CC(C)(C)OC(=O)CC(=O)[C@@H](NC(=O)OCc1ccccc1)C1CCCC(F)(F)C1 ; CC(C)(C)OC(=O)C(Br)C(=O)[C@@H](NC(=O)OCc1ccccc1)C1CCCC(F)(F)C1]

To a solution of tert-butyl (4S)-4-(((benzyloxy)carbonyl)amino)-4-(3,3-difluorocyclohexyl)-3-oxobutanoate (1.50 g, 3.53 mmol, 1.00 eq) in MeOH (15.0 mL) was added NBS (502 mg, 2.82 mmol, 0.800 eq) and 2,6-dimethylpyidine (30.2 mg, 282 μmol, 32.8 μL, 0.0800 eq) at 0° C. The reaction mixture was stirred at RT for 2 h. The reaction mixture was concentrated under reduced pressure to remove MeOH. The residue was purified by column chromatography (SiO₂, PE:EtOAc=1:0 to 10:1). The product, tert-butyl (4S)-4-(((benzyloxy)carbonyl)amino)-2-bromo-4-(3,3-difluorocyclohexyl)-3-oxobutanoate (1.50 g, 2.97 mmol, 84% yield), was obtained as a yellow oil. LCMS [M−99]⁺=406.1 m/z.

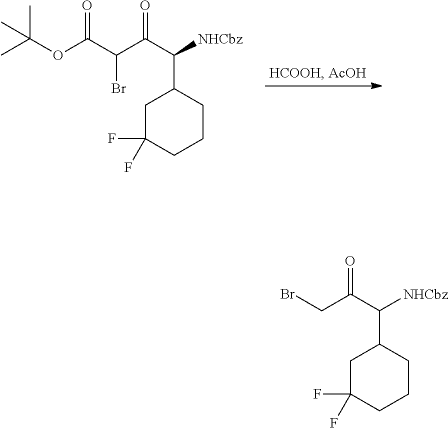 [SMILES: CC(C)(C)OC(=O)C(Br)C(=O)[C@@H](NC(=O)OCc1ccccc1)C1CCCC(F)(F)C1 ; O=C(NC(C(=O)CBr)C1CCCC(F)(F)C1)OCc1ccccc1]

A solution of tert-butyl (4S)-4-(((benzyloxy)carbonyl)amino)-2-bromo-4-(3,3-difluorocyclohexyl)-3-oxobutanoate (1.00 g, 1.98 mmol, 1.00 eq) in HCOOH (95.2 mg, 1.98 mmol, 5.00 mL, 1.00 eq) and AcOH (5.25 g, 87.4 mmol, 5.00 mL, 44.1 eq) was stirred at 80° C. for 2 h. The residue was diluted with saturated aqueous NaHCO₃ to pH=7 and extracted with EtOAc (60.0 mL×3). The combined organic layers were dried over Na₂SO₄, filtered, and concentrated under reduced pressure to give a residue. The residue was purified by column chromatography (SiO₂, PE:EtOAc=20:1 to 5:1). The product, benzyl (3-bromo-1-(3,3-difluorocyclohexyl)-2-oxopropyl)carbamate (1.10 g, crude), was obtained as a yellow solid. SFC shows a mixture of 4 stereoisomers (1/1/1/1). LCMS [M+H]⁺=406.2 m/z.

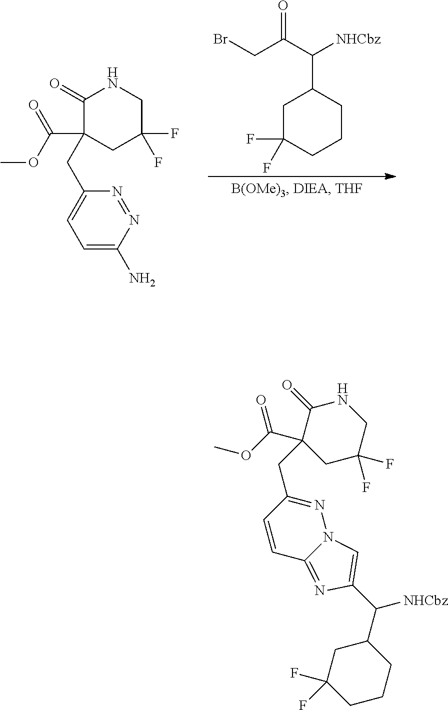 [SMILES: COC(=O)C1(Cc2ccc(N)nn2)CC(F)(F)CNC1=O ; O=C(NC(C(=O)CBr)C1CCCC(F)(F)C1)OCc1ccccc1 ; COC(=O)C1(Cc2ccc3nc(C(NC(=O)OCc4ccccc4)C4CCCC(F)(F)C4)cn3n2)CC(F)(F)CNC1=O]

To a solution of methyl 3-((6-aminopyridazin-3-yl)methyl)-5,5-difluoro-2-oxopiperidine-3-carboxylate (100 mg, 241 μmol, 1.00 eq, TFA salt) and benzyl (3-bromo-1-(3,3-difluorocyclohexyl)-2-oxopropyl)carbamate (202 mg, 499 μmol, 2.1 eq) in THF (4.00 mL) was added B(OMe)₃ (207 mg, 2.00 mmol, 225 μL, 8.3 eq) and DIEA (258 mg, 2.00 mmol, 348 μL, 8.3 eq). The reaction mixture was stirred at 70° C. for 4 h. The reaction mixture was diluted with water (30.0 mL) and extracted with EtOAc (30.0 mL×2). The combined organic layers were washed with brine 30.0 mL, dried over Na₂SO₄, filtered, and concentrated under reduced pressure to give a residue. The residue was purified by prep-TLC (SiO₂, PE:EtOAc=0:1, R_{f}=0.38). Methyl 3-((2-((((benzyloxy)carbonyl)amino)(3,3-difluorocyclohexyl)methyl)imidazo[1,2-b]pyridazin-6-yl)methyl)-5,5-difluoro-2-oxopiperidine-3-carboxylate (160 mg, crude) was obtained as a yellow oil and used directly in the next step. LCMS [M+H]⁺=606.4 m/z.

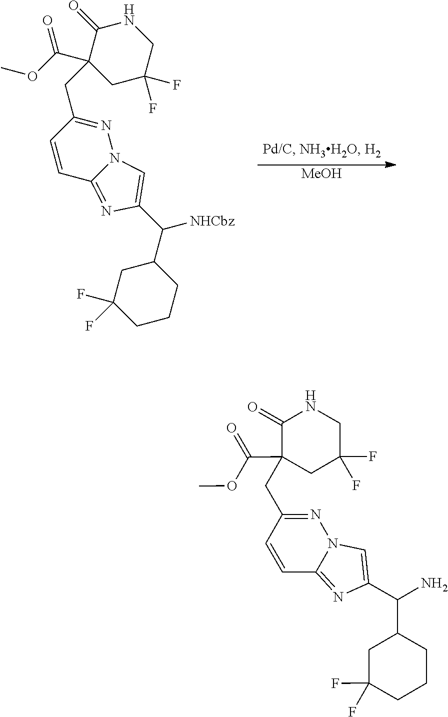 [SMILES: "MeOH" ; COC(=O)C1(Cc2ccc3nc(C(NC(=O)OCc4ccccc4)C4CCCC(F)(F)C4)cn3n2)CC(F)(F)CNC1=O ; COC(=O)C1(Cc2ccc3nc(C(N)C4CCCC(F)(F)C4)cn3n2)CC(F)(F)CNC1=O]

To a solution of methyl 3-((2-((((benzyloxy)carbonyl)amino)(3,3-difluorocyclohexyl)methyl)imidazo[1,2-b]pyridazin-6-yl)methyl)-5,5-difluoro-2-oxopiperidine-3-carboxylate (150 mg, crude) in MeOH (3.00 mL) was added Pd/C (15.0 mg, 10.0% purity) and NH₃·H₂O (1.09 g, 7.79 mmol, 1.20 mL, 25.0% purity, 31.4 eq), and then the reaction mixture was stirred at RT for 1 h under H₂ (15.0 psi) atmosphere. The reaction mixture was filtered, and the filtrate was concentrated under reduced pressure. The crude product, methyl 3-((2-(amino(3,3-difluorocyclohexyl)methyl)imidazo[1,2-b]pyridazin-6-yl)methyl)-5,5-difluoro-2-oxopiperidine-3-carboxylate (110 mg, crude) was obtained as a yellow oil and used directly in the next step. LCMS [M+H]⁺=472.3 m/z.

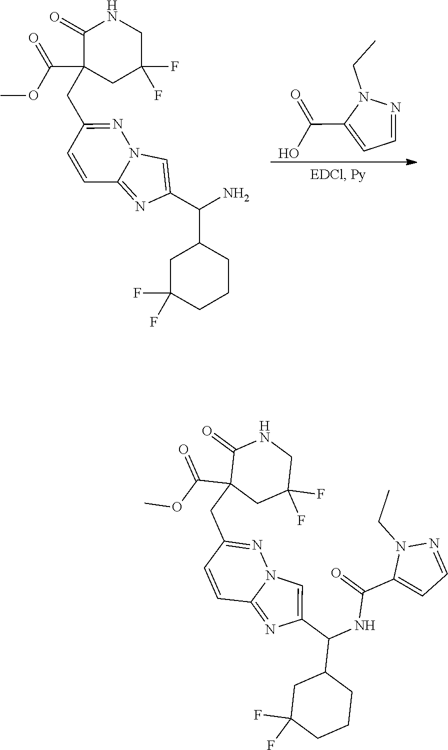

To a solution of methyl 3-((2-(amino(3,3-difluorocyclohexyl)methyl)imidazo[1,2-b]pyridazin-6-yl)methyl)-5,5-difluoro-2-oxopiperidine-3-carboxylate (100 mg crude) and 1-ethyl-1H-pyrazole-5-carboxylic acid (44.6 mg, 318 μmol, 1.50 eq) in pyridine (2.00 mL) was added EDCI (162 mg, 848 μmol, 4.00 eq). The reaction mixture was stirred at RT for 2 h. The reaction mixture was diluted with H₂O (20.0 mL) and extracted with DCM (20.0 mL×3). The combined organic phases were dried over Na₂SO₄ and concentrated to give a residue. The residue was purified by prep-TLC (SiO₂, PE:EtOAc=0:1, R_{f}=0.45). The product, methyl 3-((2-((3,3-difluorocyclohexyl)(1-ethyl-1H-pyrazole-5-carboxamido)methyl)imidazo[1,2-b]pyridazin-6-yl)methyl)-5,5-difluoro-2-oxopiperidine-3-carboxylate (80.0 mg, 134 μmol, 59% yield over 3 steps), was obtained as a yellow oil. LCMS [M+H]⁺=594.1 m/z.

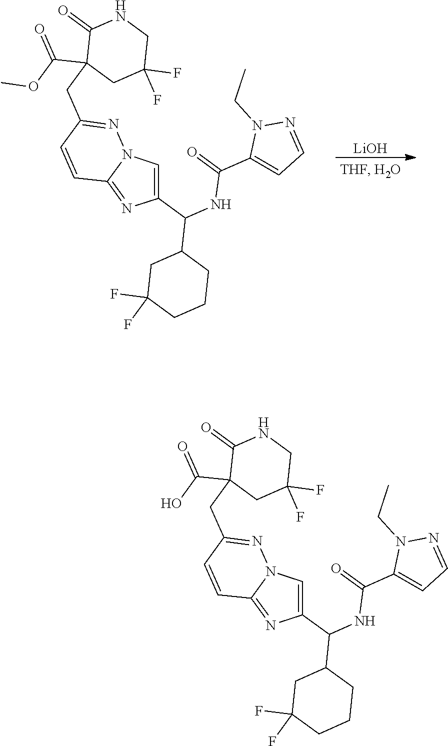

To a solution of methyl 3-((2-((3,3-difluorocyclohexyl)(1-ethyl-1H-pyrazole-5-carboxamido)methyl)imidazo[1,2-b]pyridazin-6-yl)methyl)-5,5-difluoro-2-oxopiperidine-3-carboxylate (70.0 mg, 118 μmol, 1.00 eq) in THF (3.00 mL) and H₂O (1.00 mL) was added LiOH·H₂O (19.8 mg, 471.7 μmol, 4.00 eq). The reaction mixture was stirred at RT for 30 min. The reaction mixture was acidified to pH=5 with HCl (1.00 M) and concentrated under reduced pressure to give a residue. The product, 3-((2-((3,3-difluorocyclohexyl)(1-ethyl-1H-pyrazole-5-carboxamido)methyl)imidazo[1,2-b]pyridazin-6-yl)methyl)-5,5-difluoro-2-oxopiperidine-3-carboxylic acid (70.0 mg, crude), was obtained as a colorless oil. LCMS [M+H]⁺=579.9 m/z.

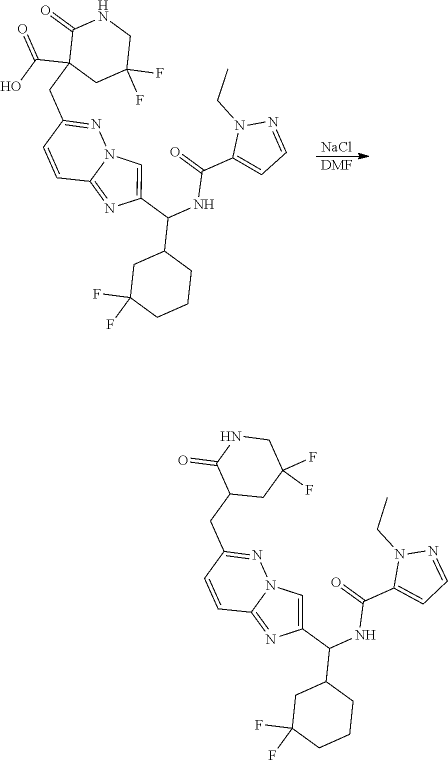

To a solution of 3-((2-((3,3-difluorocyclohexyl)(1-ethyl-1H-pyrazole-5-carboxamido)methyl)imidazo[1,2-b]pyridazin-6-yl)methyl)-5,5-difluoro-2-oxopiperidine-3-carboxylic acid (60.0 mg) in DMF (1.00 mL) was added NaCl (18.1 mg, 310 μmol, 3.00 eq). The reaction mixture was stirred at 110° C. for 1 h. The reaction mixture was concentrated under reduced pressure to give a residue. The residue was purified by prep-TLC (SiO₂, PE:EtOAc=0:1, R_{f}=0.40). The mixture of stereoisomers, N-((6-((5,5-difluoro-2-oxopiperidin-3-yl)methyl)imidazo[1,2-b]pyridazin-2-yl)(3,3-difluorocyclohexyl)methyl)-1-ethyl-1H-pyrazole-5-carboxamide (Compound 32, 43.78 mg, 79.8 μmol, 79% yield over 2 steps), was obtained as a white solid. LCMS [M+H]⁺=536.2 m/z.

### Example 45: Preparation of Compounds 33 and 34

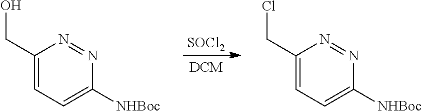 [SMILES: CC(C)(C)OC(=O)Nc1ccc(CO)nn1 ; "SOCl2" ; CC(C)(C)OC(=O)Nc1ccc(CCl)nn1]

To a solution of tert-butyl (6-(hydroxymethyl)pyridazin-3-yl)carbamate (1.00 g, 4.44 mmol, 1.00 eq) in DCM (10.0 mL) was added SOCl₂ (2.64 g, 22.2 mmol, 1.61 mL, 5.00 eq) at 0° C. The reaction mixture was stirred at RT for 3 h. The reaction mixture was concentrated under reduced pressure to give a residue. Crude product, tert-butyl (6-(chloromethyl)pyridazin-3-yl)carbamate (1.00 g, crude), was obtained as a white solid. LCMS [M+H]⁺=244.0 m/z.

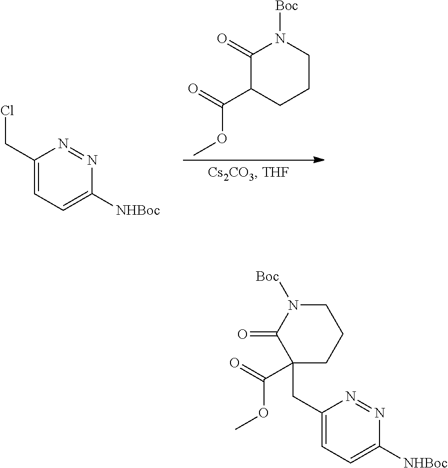 [SMILES: CC(C)(C)OC(=O)Nc1ccc(CCl)nn1 ; COC(=O)C1CCCN(C(=O)OC(C)(C)C)C1=O ; COC(=O)C1(Cc2ccc(NC(=O)OC(C)(C)C)nn2)CCCN(C(=O)OC(C)(C)C)C1=O]

To a solution of tert-butyl (6-(chloromethyl)pyridazin-3-yl)carbamate (350 mg) and 1-(tert-butyl) 3-methyl 2-oxopiperidine-1,3-dicarboxylate (406 mg, 1.58 mmol, 1.10 eq) in THE (10.0 mL) was added Cs₂CO₃ (1.40 g, 4.31 mmol, 3.00 eq). The reaction mixture was stirred at 70° C. for 2 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. Crude product, 1-(tert-butyl)3-methyl 3-((6-((tert-butoxycarbonyl)amino)pyridazin-3-yl)methyl)-2-oxopiperidine-1,3-dicarboxylate (600 mg, crude), was obtained as a white solid. LCMS [M+H]⁺=465.2 m/z.

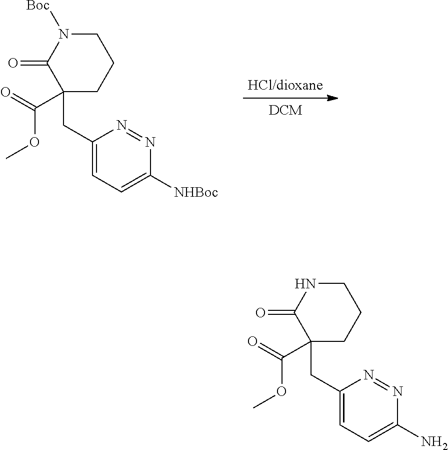 [SMILES: COC(=O)C1(Cc2ccc(NC(=O)OC(C)(C)C)nn2)CCCN(C(=O)OC(C)(C)C)C1=O ; COC(=O)C1(Cc2ccc(N)nn2)CCCNC1=O]

To a solution of 1-(tert-butyl) 3-methyl 3-((6-((tert-butoxycarbonyl)amino)pyridazin-3-yl)methyl)-2-oxopiperidine-1,3-dicarboxylate (600 mg, crude) in DCM (5.00 mL) was added HCl/dioxane (4.00 M, 3.60 mL, 11.1 eq). The reaction mixture was stirred at RT for 3 h. The reaction mixture was concentrated under reduced pressure to give a residue. The crude product, methyl 3-((6-aminopyridazin-3-yl)methyl)-2-oxopiperidine-3-carboxylate (340 mg, crude, HCl salt), was obtained as a white solid. LCMS [M+H]⁺=265.2 m/z.

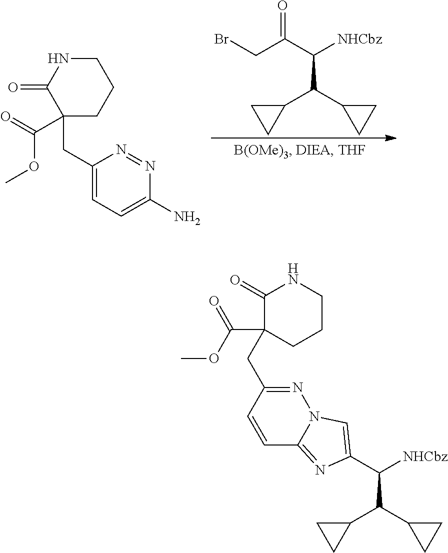 [SMILES: COC(=O)C1(Cc2ccc(N)nn2)CCCNC1=O ; O=C(N[C@H](C(=O)CBr)C(C1CC1)C1CC1)OCc1ccccc1 ; COC(=O)C1(Cc2ccc3nc([C@@H](NC(=O)OCc4ccccc4)C(C4CC4)C4CC4)cn3n2)CCCNC1=O]

To a solution of methyl 3-((6-aminopyridazin-3-yl)methyl)-2-oxopiperidine-3-carboxylate (340 mg) and Intermediate 1 (489 mg, 1.29 mmol, 1.00 eq) in THF (8.00 mL) was added DIEA (1.48 g, 11.4 mmol, 2.00 mL, 8.93 eq) and B(OMe)₃ (1.34 g, 12.8 mmol, 1.45 mL, 10.0 eq). The reaction mixture was stirred at 70° C. for 3 h under N₂. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The residue was purified by prep-TLC (SiO₂, DCM:MeOH=10/1, monitoring, DCM:MeOH=10:1, Rf (P1)=0.34). Methyl 3-((2-((S)-1-(((benzyloxy)carbonyl)amino)-2,2-dicyclopropylethyl)imidazo[1,2-b]pyridazin-6-yl)methyl)-2-oxopiperidine-3-carboxylate (600 mg, crude) was obtained as a white solid. LCMS [M+H]⁺=546.3 m/z.

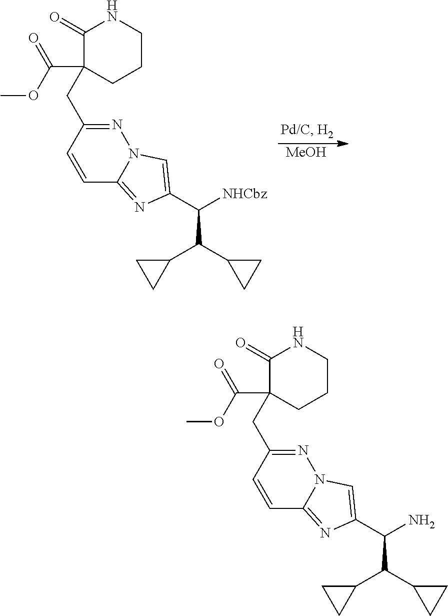 [SMILES: "MeOH" ; COC(=O)C1(Cc2ccc3nc([C@@H](NC(=O)OCc4ccccc4)C(C4CC4)C4CC4)cn3n2)CCCNC1=O ; COC(=O)C1(Cc2ccc3nc([C@@H](N)C(C4CC4)C4CC4)cn3n2)CCCNC1=O]

To a solution of methyl 3-((2-((S)-1-(((benzyloxy)carbonyl)amino)-2,2-dicyclopropylethyl)imidazo[1,2-b]pyridazin-6-yl)methyl)-2-oxopiperidine-3-carboxylate (200 mg) in MeOH (10.0 mL) was added Pd/C (35.0 mg, 10.0% purity). The reaction mixture was stirred at RT for 2 h under H₂ (15 psi). The reaction mixture was filtered and concentrated under reduced pressure to give a residue. The crude product, methyl 3-((2-((S)-1-amino-2,2-dicyclopropylethyl)imidazo[1,2-b]pyridazin-6-yl)methyl)-2-oxopiperidine-3-carboxylate (150 mg, crude), was obtained as a white solid. LCMS [M+H]⁺=412.1 m/z.

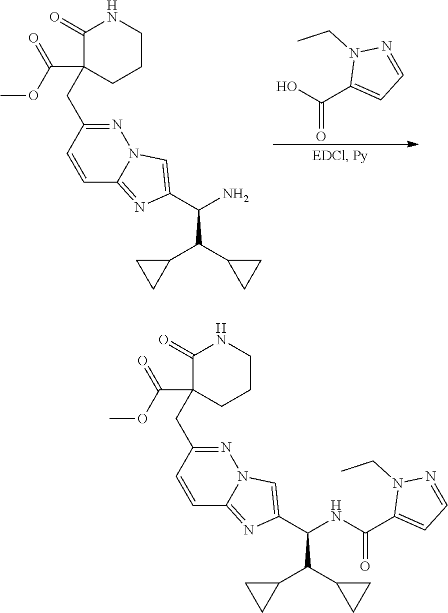

To a solution of methyl 3-((2-((S)-1-amino-2,2-dicyclopropylethyl)imidazo[1,2-b]pyridazin-6-yl)methyl)-2-oxopiperidine-3-carboxylate (0.150 g, 364 μmol, 1.00 eq) and 1-ethyl-1H-pyrazole-5-carboxylic acid (66.4 mg, 473 μmol, 1.30 eq) in pyridine (5.00 mL) was added EDCI (209 mg, 1.09 mmol, 3.00 eq). The reaction mixture was stirred at RT for 3 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The residue was purified by prep-TLC (SiO₂, DCM:MeOH=10:1, monitoring, DCM:MeOH=10:1, Rf(P1)=0.34). The product, methyl 3-((2-((S)-2,2-dicyclopropyl-1-(1-ethyl-1H-pyrazole-5-carboxamido)ethyl)imidazo[1,2-b]pyridazin-6-yl)methyl)-2-oxopiperidine-3-carboxylate (150 mg, 281 μmol, 54% yield over 6 steps) was obtained as a white solid. LCMS [M+H]⁺=534.4 m/z.

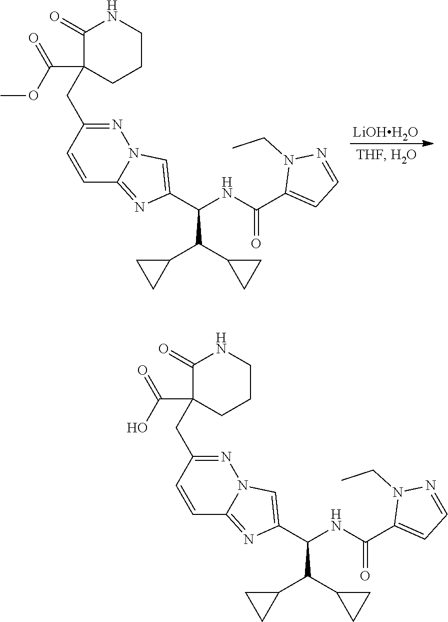

To a solution of methyl 3-((2-((S)-2,2-dicyclopropyl-1-(1-ethyl-1H-pyrazole-5-carboxamido)ethyl)imidazo[1,2-b]pyridazin-6-yl)methyl)-2-oxopiperidine-3-carboxylate (0.13 g 243 μmol, 1.00 eq) in THF (3.00 mL) and H₂O (1.00 mL) was added LiOH·H₂O (102 mg, 2.44 mmol, 10.0 eq). The reaction mixture was stirred at RT for 3 h. The combined reaction mixture was washed with EtOAc (20.0 mL×2). The pH of the aqueous phase was adjusted to 3 with 1 M HCl and extracted with EtOAc (20.0 mL×2). The combined organic layers were dried over Na₂SO₄, filtered, and concentrated under reduced pressure to give a residue. The product, 3-((2—((S)-2,2-dicyclopropyl-1-(1-ethyl-1H-pyrazole-5-carboxamido)ethyl)imidazo[1,2-b]pyridazin-6-yl)methyl)-2-oxopiperidine-3-carboxylic acid (0.12 g, crude), was obtained as a white solid. LCMS [M+H]⁺=520.1 m/z.

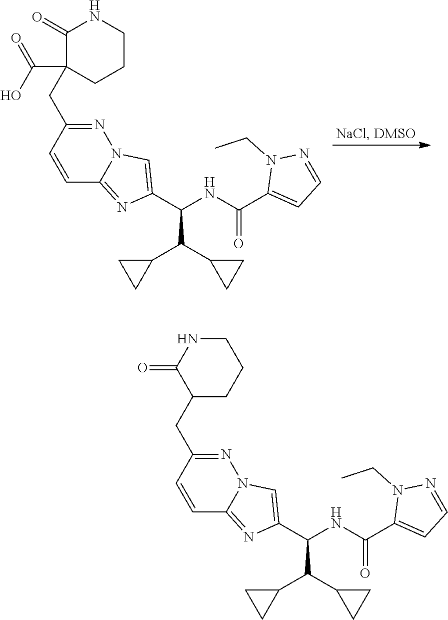 [SMILES: CCn1nccc1C(=O)N[C@H](c1cn2nc(CC3(C(=O)O)CCCNC3=O)ccc2n1)C(C1CC1)C1CC1 ; CCn1nccc1C(=O)N[C@H](c1cn2nc(CC3CCCNC3=O)ccc2n1)C(C1CC1)C1CC1]

To a solution of 3-((2-((S)-2,2-dicyclopropyl-1-(1-ethyl-1H-pyrazole-5-carboxamido)ethyl)imidazo[1,2-b]pyridazin-6-yl)methyl)-2-oxopiperidine-3-carboxylic acid (120 mg) in DMSO (6.00 mL) was added NaCl (67.4 mg, 1.15 mmol, 5.00 eq). The reaction mixture was stirred at 130° C. for 1 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The residue was purified by prep-TLC (SiO₂, DCM:MeOH=10:1, Rf(P1)=0.34) and prep-SFC (DAICEL CHIRALPAK AD (250 mm×30 mm, 10 μm); 40% mobile phase: [0.1% NH₃H₂O IPA]). The first eluting stereoisomer, N—((S)-2,2-dicyclopropyl-1-(6-(((R)-2-oxopiperidin-3-yl)methyl)imidazo[1,2-b]pyridazin-2-yl)ethyl)-1-ethyl-1H-pyrazole-5-carboxamide (Compound 34, 29.4 mg, 61.3 μmol, 25% yield over 2 steps), verified by X-ray crystallographic analysis of intermediate (R)-3-((6-aminopyridazin-3-yl)methyl)piperidin-2-one and subsequent semi-synthesis by General Procedure 2, was obtained as a yellow solid. LCMS [M+H]⁺=476.3 m/z. By deduction, the second eluting stereoisomer was found to have the absolute configuration, N—((S)-2,2-dicyclopropyl-1-(6-(((S)-2-oxopiperidin-3-yl)methyl)imidazo[1,2-b]pyridazin-2-yl)ethyl)-1-ethyl-1H-pyrazole-5-carboxamide (Compound 33, 37.3 mg, 75.9 μmol, 31% yield over 2 steps), obtained as a yellow solid. LCMS [M+H]⁺=476.2 m/z.

### Example 46: Preparation of Compound 35

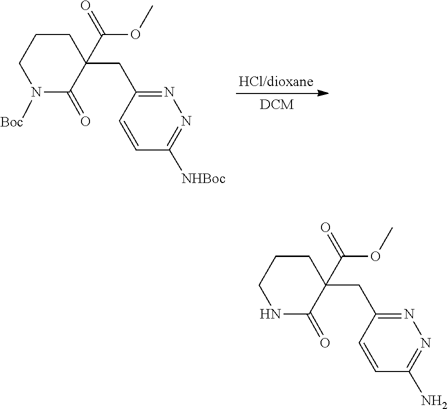 [SMILES: COC(=O)C1(Cc2ccc(NC(=O)OC(C)(C)C)nn2)CCCN(C(=O)OC(C)(C)C)C1=O ; COC(=O)C1(Cc2ccc(N)nn2)CCCNC1=O]

To a solution of 1-(tert-butyl) 3-methyl 3-((6-((tert-butoxycarbonyl)amino)pyridazin-3-yl)methyl)-2-oxopiperidine-1,3-dicarboxylate (30.5 g, 65.6 mmol, 1.00 eq) in DCM (250 mL) was added HCl/dioxane (4.00 M, 100 mL, 6.09 eq) at 0° C. The reaction mixture was stirred at 30° C. for 18 h. The reaction mixture was concentrated under reduced pressure to afford crude methyl 3-((6-aminopyridazin-3-yl)methyl)-2-oxopiperidine-3-carboxylate (20.0 g, crude, HCl salt) as yellow solid. LCMS [M+H]⁺=265.1 m/z.

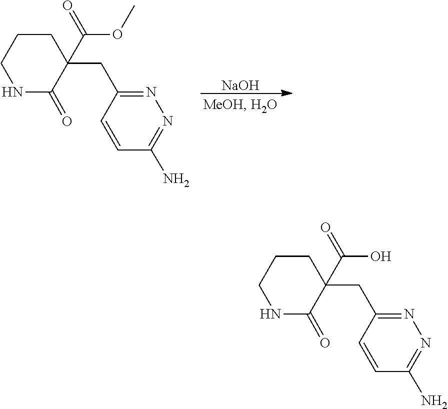 [SMILES: COC(=O)C1(Cc2ccc(N)nn2)CCCNC1=O ; "NaOH" ; Nc1ccc(CC2(C(=O)O)CCCNC2=O)nn1]

To a solution of methyl 3-((6-aminopyridazin-3-yl)methyl)-2-oxopiperidine-3-carboxylate (20.0 g, 66.5 mmol, 1.00 eq, HCl) in THF (200 mL) was added a solution of NaOH (7.98 g, 199 mmol, 3.00 eq) in H₂O (60.0 mL) at 0° C. The reaction mixture was stirred at 30° C. for 12 h. The reaction mixture was adjusted with 1.00 M HCl to pH=5 and concentrated under reduced pressure to afford crude 3-((6-aminopyridazin-3-yl)methyl)-2-oxopiperidine-3-carboxylic acid (20.0 g, crude) as yellow solid. LCMS [M+H]⁺=251.0 m/z.

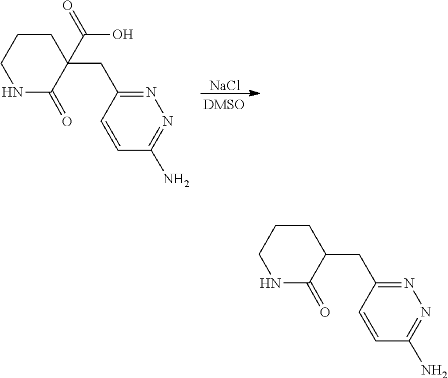 [SMILES: Nc1ccc(CC2(C(=O)O)CCCNC2=O)nn1 ; "NaCl" ; Nc1ccc(CC2CCCNC2=O)nn1]

To a solution of 3-((6-aminopyridazin-3-yl)methyl)-2-oxopiperidine-3-carboxylic acid (20 g, crude) in DMF (1 M) was added NaCl (3.00 eq), carried out in 2 separate batches. The reaction mixtures were stirred at 110° C. for 10 h. The reaction mixtures were then combined, filtered with MeOH washes, and the filtrate was concentrated to give a residue. The residue was purified by prep-HPLC (Kromasil Eternity XT 250×80 mm×10 μm; mobile phase: [water (NH₃H₂O v/v)-ACN]; B %: 0%-20%, 20 min, HPLC) to afford a mixture of stereoisomers, 3-((6-aminopyridazin-3-yl)methyl)piperidin-2-one (12 g, 58.1 mmol, 89% yield over 2 steps), as yellow solid. LCMS [M+Na]⁺=229.1 m/z. ¹H NMR (400 MHz, CDCl₃) δ 7.30 (d, J=9.2 Hz, 1H), 6.90 (d, J=9.2 Hz, 1H), 3.31-3.22 (m, 3H), 2.92-2.86 (m, 1H), 2.74-2.68 (m, 1H), 1.87-1.76 (m, 2H), 1.92-1.50 (m, 2H).

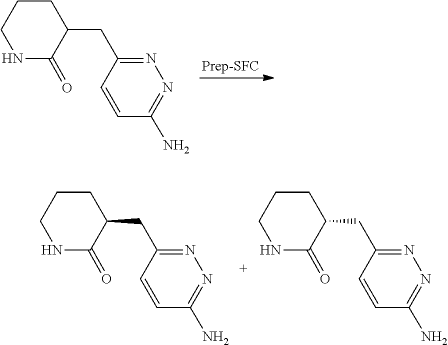 [SMILES: Nc1ccc(CC2CCCNC2=O)nn1 ; Nc1ccc(C[C@@H]2CCCNC2=O)nn1 ; Nc1ccc(C[C@H]2CCCNC2=O)nn1]

Compound 7 was separated by SFC (DAICEL CHIRALPAK IC (250 mm×50 mm, 10 μm); mobile phase: [ACN/MeOH (0.1% NH₃H₂O)]; B %: 50%-50%, 4.5 min,). The residue was purified by reversed-phase HPLC (0.1% NH₃·H₂O). The residue was purified by reversed-phase HPLC (0.1% NH₃·H₂O) to afford a white solid, (S)-3-((6-aminopyridazin-3-yl)methyl)piperidin-2-one (5.75 g, 27.8 mmol, 43% yield), as the first eluting enantiomer from prep-SFC. LCMS [M+H]⁺=207.2 m/z. The second eluting enantiomer, (R)-3-((6-aminopyridazin-3-yl)methyl)piperidin-2-one (5.50 g, 26.6 mmol, 41% yield), was obtained as brown solid, confirmed by X-ray crystallographic analysis. LCMS [M+H]⁺=207.1 m/z.

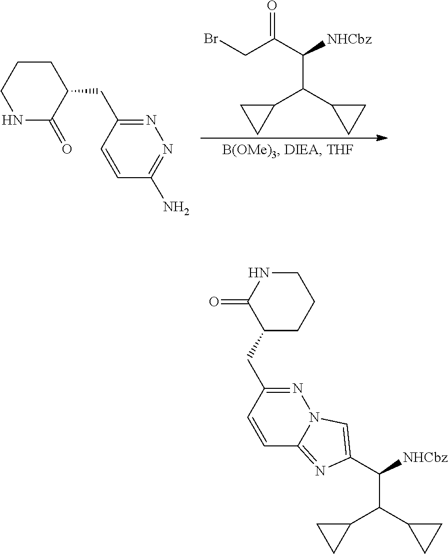

Benzyl ((S)-2,2-dicyclopropyl-1-(6-(((R)-2-oxopiperidin-3-yl)methyl)imidazo[1,2-b]pyridazin-2-yl)ethyl)carbamate was synthesized following General Procedure 1, using (R)-3-((6-aminopyridazin-3-yl)methyl)piperidin-2-one (1.30 g, 6.30 mmol, 1.00 eq), Intermediate 1 (2.64 g, 6.93 mmol, 1.10 eq), B(OMe)₃ (3.27 g, 31.5 mmol, 3.56 mL, 5.00 eq), and DIEA (8.15 g, 638.0 mmol, 10.9 mL, 10.0 eq). After 2 h at 70° C., crude product was purified by prep-HPLC (Kromasil Eternity XT 250×80 mm×10 μm; mobile phase: [water (NH₃H₂O v/v)-ACN]; B %: 38%-68%). Combined fractions were concentrated to remove MeCN and extracted with EtOAc (80.0 mL×3). The organic layers were dried over anhydrous Na₂SO₄, filtered, and concentrated under reduced pressure to afford benzyl ((S)-2,2-dicyclopropyl-1-(6-(((R)-2-oxopiperidin-3-yl)methyl)imidazo[1,2-b]pyridazin-2-yl)ethyl)carbamate (3.00 g, 6.15 mmol, 98% yield) as a yellow solid. LCMS [M+H]⁺=488.3 m/z.

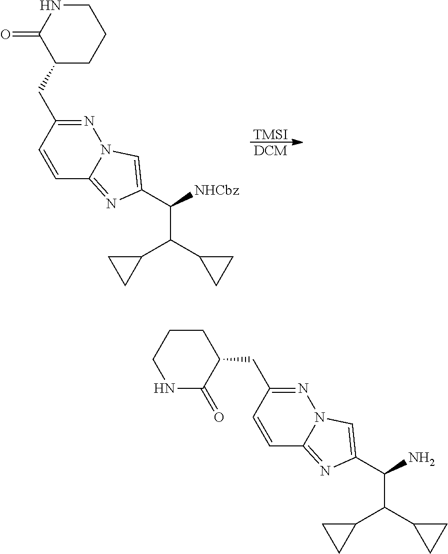

To a solution of benzyl ((S)-2,2-dicyclopropyl-1-(6-(((R)-2-oxopiperidin-3-yl)methyl)imidazo[1,2-b]pyridazin-2-yl)ethyl)carbamate (150 mg, 303 μmol, 1.00 eq) in DCM (6.00 mL) was added TMSI (90.9 mg, 454 μmol, 61.8 μL, 1.50 eq) at 0° C. The reaction mixture was stirred at 0° C. for 2 h. The residue was diluted with water (30.0 mL) and adjusted with HCl (1 M) to pH=3, then washed with EtOAc (30.0 mL×2). The aqueous phase was adjusted to pH=9 with saturated aqueous NaHCO₃ and extracted with EtOAc (40.0 mL×3). The combined organic layers were dried over anhydrous Na₂SO₄, filtered, and concentrated under reduced pressure to afford (R)-3-((2-((S)-1-amino-2,2-dicyclopropylethyl)imidazo[1,2-b]pyridazin-6-yl)methyl)piperidin-2-one (80.0 mg, 226 μmol, 75% yield) as yellow oil. LCMS [M+H]⁺=354.3 m/z.

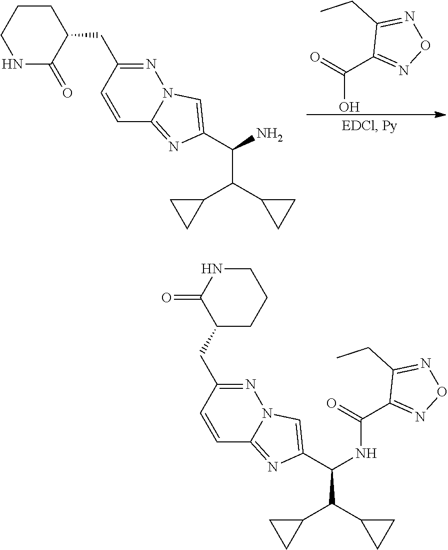

The title compound was synthesized following General Procedure 4, using (R)-3-((2-((S)-1-amino-2,2-dicyclopropylethyl)imidazo[1,2-b]pyridazin-6-yl)methyl)piperidin-2-one (40.0 mg 113 μmol, 1.00 eq), 4-ethyl-1,2,5-oxadiazole-3-carboxylic acid (24.0 mg, 170 μmol, 1.50 eq), EDCI (65.1 mg, 339 μmol, 3.00 eq), and pyridine (1.50 mL). The reaction mixture was stirred for 16 h. The residue was purified by prep-HPLC (Phenomenex luna C18.0 150×25.0 mm×10.0 um; mobile phase: [water (FA)-ACN]; B %: 43.0%-73.0%, 10.0 min). The product, N—((S)-2,2-dicyclopropyl-1-(6-(((R)-2-oxopiperidin-3-yl)methyl)imidazo[1,2-b]pyridazin-2-yl)ethyl)-4-ethyl-1,2,5-oxadiazole-3-carboxamide (Compound 35, 13.9 mg, 28.5 μmol, 25% yield), was obtained as a white solid. LCMS [M+H]⁺=478.3 m/z.

### Example 47: Preparation of Compound 36

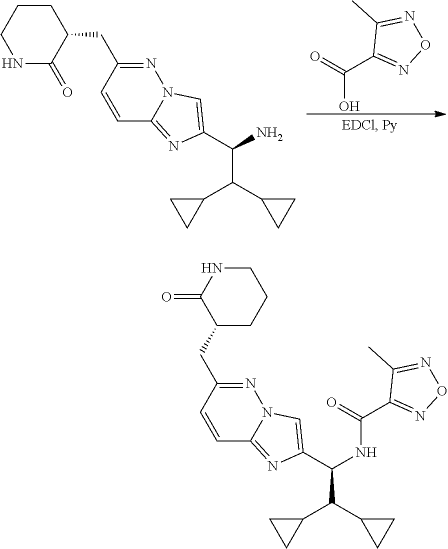

The title compound was synthesized following General Procedure 4, using (R)-3-((2-((S)-1-amino-2,2-dicyclopropylethyl)imidazo[1,2-b]pyridazin-6-yl)methyl)piperidin-2-one (40.0 mg 113 μmol, 1.00 eq), 4-methyl-1,2,5-oxadiazole-3-carboxylic acid (21.7 mg, 169 μmol, 1.50 eq), EDCI (65.9 mg, 339 μmol, 3.00 eq), and pyridine (1.50 mL). The reaction mixture was stirred for 6 h. The residue was purified by prep-HPLC (Phenomenex luna C18 150×25 mm×10 μm; mobile phase: [water (FA)-CAN]; B %: 38.0%-68.0%, 10.0 min). The product, N—((S)-2,2-dicyclopropyl-1-(6-(((R)-2-oxopiperidin-3-yl)methyl)imidazo[1,2-b]pyridazin-2-yl)ethyl)-4-methyl-1,2,5-oxadiazole-3-carboxamide, (Compound 36, 20.9 mg, 44.7 μmol, 40% yield), was obtained as white solid. LCMS [M+H]⁺=464.3 m/z.

### Example 48: Preparation of Compound 37

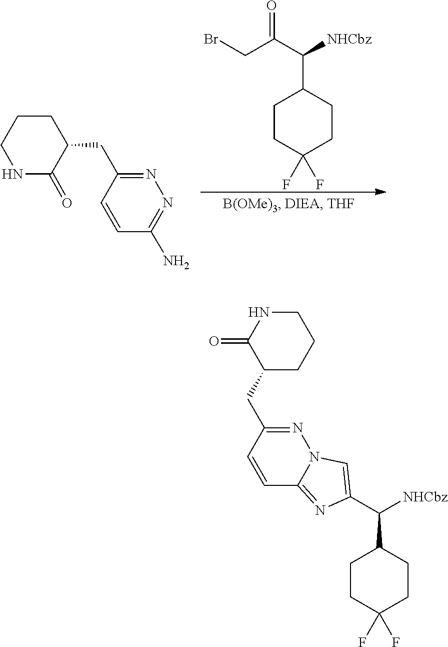 [SMILES: Nc1ccc(C[C@H]2CCCNC2=O)nn1 ; O=C(N[C@H](C(=O)CBr)C1CCC(F)(F)CC1)OCc1ccccc1 ; O=C(N[C@H](c1cn2nc(C[C@H]3CCCNC3=O)ccc2n1)C1CCC(F)(F)CC1)OCc1ccccc1]

Benzyl ((S)-(4,4-difluorocyclohexyl)(6-(((R)-2-oxopiperidin-3-yl)methyl)imidazo[1,2-b]pyridazin-2-yl)methyl)carbamate was synthesized following General Procedure 1, using (R)-3-((6-aminopyridazin-3-yl)methyl)piperidin-2-one (100 mg, 484 μmol, 1.00 eq), Intermediate 2 (235 mg, 5814 μmol, 1.20 eq), B(OMe)₃ (251 mg, 2.42 mmol, 273 μL, 5.00 eq), and DIEA (626 mg, 4.85 mmol, 844 μL, 10.0 eq). After 3 h at 70° C., crude product was purified by prep-TLC (SiO₂, EtOAc/MeOH=8:1). The product, benzyl ((S)-(4,4-difluorocyclohexyl)(6-(((R)-2-oxopiperidin-3-yl)methyl)imidazo[1,2-b]pyridazin-2-yl)methyl)carbamate (180 mg, 351 μmol, 73% yield), was obtained as a yellow solid. LCMS [M+H]⁺=512.3 m/z.

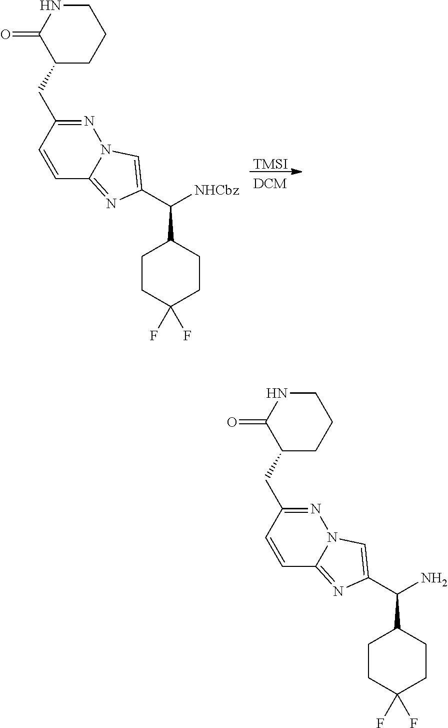 [SMILES: "TMSI" ; O=C(N[C@H](c1cn2nc(C[C@H]3CCCNC3=O)ccc2n1)C1CCC(F)(F)CC1)OCc1ccccc1 ; N[C@H](c1cn2nc(C[C@H]3CCCNC3=O)ccc2n1)C1CCC(F)(F)CC1]

Intermediate (R)-3-((2-((S)-amino(4,4-difluorocyclohexyl)methyl)imidazo[1,2-b]pyridazin-6-yl)methyl)piperidin-2-one was synthesized following General Procedure 2, using benzyl ((S)-(4,4-difluorocyclohexyl)(6-(((R)-2-oxopiperidin-3-yl)methyl)imidazo[1,2-b]pyridazin-2-yl)methyl)carbamate (120 mg, 234 μmol, 1.00 eq), TMSI (401 mg, 2.01 mmol, 273 μL, 8.55 eq), and DCM (4.00 mL). The product, (R)-3-((2-((S)-amino(4,4-difluorocyclohexyl)methyl)imidazo[1,2-b]pyridazin-6-yl)methyl)piperidin-2-one (70 mg, 185 μmol, 79% yield), was obtained as a yellow solid. LCMS [M+H]⁺=378.2 m/z.

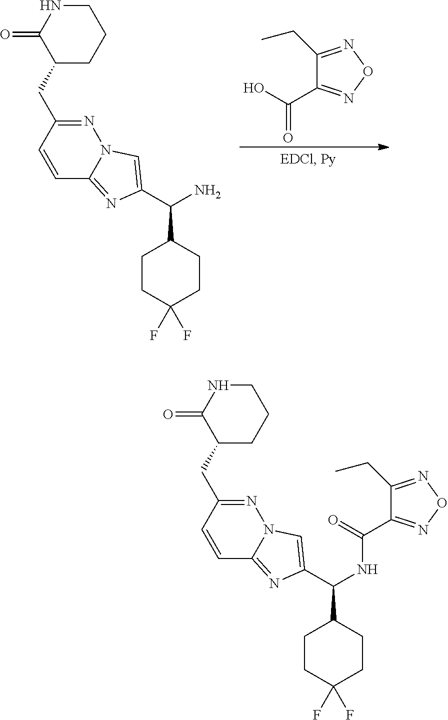

The title compound was synthesized following General Procedure 4, using (R)-3-((2-((S)-amino(4,4-difluorocyclohexyl)methyl)imidazo[1,2-b]pyridazin-6-yl)methyl)piperidin-2-one (30.0 mg, 79.4 μmol, 1.00 eq), 4-ethyl-1,2,5-oxadiazole-3-carboxylic acid (13.5 mg, 95.3 μmol, 1.20 eq), EDCI (45.7 mg, 238 μmol, 3.00 eq), and pyridine (1.00 mL). The reaction mixture was stirred for 2 h. The residue was purified by prep-HPLC (UniSil 3-100 C18 Ultra (150×25 mm×3 um); mobile phase: [water (FA)-ACN]; B %: 40%-60%, 7 min). The product, N—((S)-(4,4-difluorocyclohexyl)(6-(((R)-2-oxopiperidin-3-yl)methyl)imidazo[1,2-b]pyridazin-2-yl)methyl)-4-ethyl-1,2,5-oxadiazole-3-carboxamide, (Compound 37, 13.98 mg, 27.8 μmol, 35% yield), was obtained as a white solid. LCMS [M+H]⁺=502.3 m/z.

### Example 49: Preparation of Compound 38

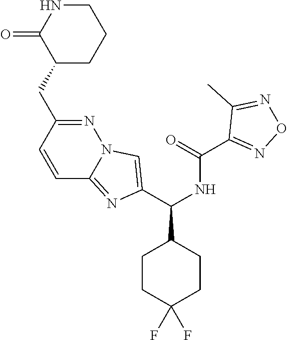 [SMILES: Cc1nonc1C(=O)N[C@H](c1cn2nc(C[C@H]3CCCNC3=O)ccc2n1)C1CCC(F)(F)CC1]

### N—((S)-(4,4-difluorocyclohexyl)(6-(((R)-2-oxopiperidin-3-yl)methyl)imidazo[1,2-b]pyridazin-2-yl)methyl)-4-methyl-1,2,5-oxadiazole-3-carboxamide. (Compound 38)

The title compound was synthesized following General Procedure 4, using (R)-3-((2-((S)-amino(4,4-difluorocyclohexyl)methyl)imidazo[1,2-b]pyridazin-6-yl)methyl)piperidin-2-one (35 mg, 92.7 μmol, 1.00 eq), 4-methyl-1,2,5-oxadiazole-3-carboxylic acid (14.2 mg, 111 μmol, 1.20 eq), EDCI (53.3 mg, 278 μmol, 3.00 eq), and pyridine (0.500 mL). The reaction mixture was stirred for 18 h. The residue was purified by prep-HPLC (Phenomenexluna C18 150×25 mm×10 μm; mobile phase: [water (FA)-ACN]; B %: 34%-64%, 10 min). The product, N—((S)-(4,4-difluorocyclohexyl)(6-(((R)-2-oxopiperidin-3-yl)methyl)imidazo[1,2-b]pyridazin-2-yl)methyl)-4-methyl-1,2,5-oxadiazole-3-carboxamide, (Compound 38, 11.18 mg, 22.8 μmol, 25% yield), was obtained as a yellow solid. LCMS [M+H]⁺=488.3 m/z.

### Example 50: Preparation of Compound 39

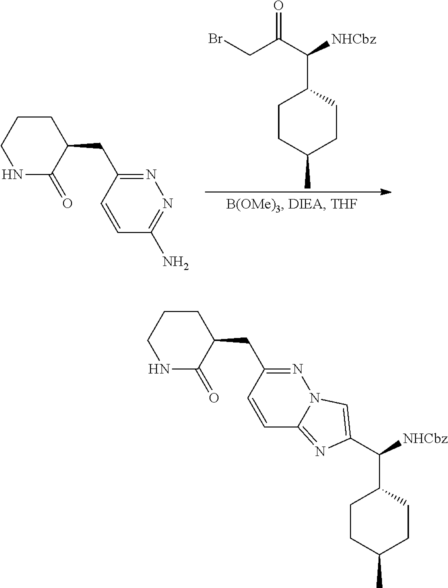 [SMILES: Nc1ccc(C[C@@H]2CCCNC2=O)nn1 ; C[C@H]1CC[C@H]([C@H](NC(=O)OCc2ccccc2)C(=O)CBr)CC1 ; C[C@H]1CC[C@H]([C@H](NC(=O)OCc2ccccc2)c2cn3nc(C[C@@H]4CCCNC4=O)ccc3n2)CC1]

Benzyl ((S)-((1r,4S)-4-methylcyclohexyl)(6-(((S)-2-oxopiperidin-3-yl)methyl)imidazo[1,2-b]pyridazin-2-yl)methyl)carbamate was synthesized following General Procedure 1, using (S)-3-((6-aminopyridazin-3-yl)methyl)piperidin-2-one (120 mg, 582 μmol, 1.00 eq), Intermediate 4 (267 mg, 698 μmol, 1.20 eq), B(OMe)₃ (302 mg, 2.91 mmol, 329 μL, 5.00 eq), and DIEA (376 mg, 2.91 mmol, 507 μL, 5.00 eq). After 3 h at 70° C., crude product was purified by prep-TLC (DCM:MeOH=10:1) to afford benzyl ((S)-((1r,4S)-4-methylcyclohexyl)(6-(((S)-2-oxopiperidin-3-yl)methyl)imidazo[1,2-b]pyridazin-2-yl)methyl)carbamate (90.0 mg, 184 μmol, 32% yield) as a light-yellow solid. LCMS [M+H]⁺=490.3 m/z.

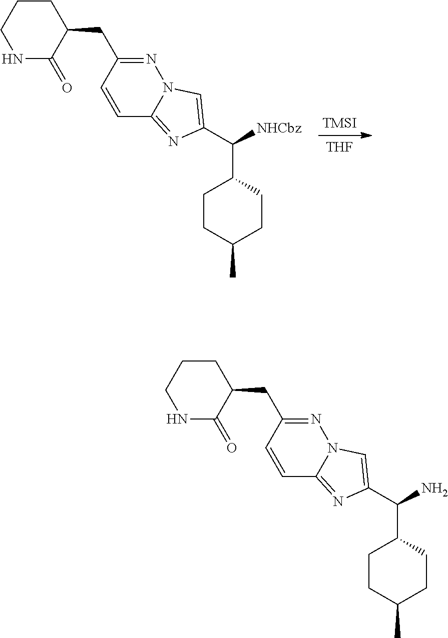 [SMILES: "TMSI" ; C[C@H]1CC[C@H]([C@H](NC(=O)OCc2ccccc2)c2cn3nc(C[C@@H]4CCCNC4=O)ccc3n2)CC1 ; C[C@H]1CC[C@H]([C@H](N)c2cn3nc(C[C@@H]4CCCNC4=O)ccc3n2)CC1 ; "THF"]

Intermediate (S)-3-((2-((S)-amino((1r,4S)-4-methylcyclohexyl)methyl)imidazo[1,2-b]pyridazin-6-yl)methyl)piperidin-2-one was synthesized following General Procedure 2, using benzyl ((S)-((1r,4S)-4-methylcyclohexyl)(6-(((S)-2-oxopiperidin-3-yl)methyl)imidazo[1,2-b]pyridazin-2-yl)methyl)carbamate (90.0 mg, 184 μmol, 1.00 eq), TMSI (110 mg, 551 μmol, 75.1 μL, 3.00 eq), and DCM (2.00 mL). The product, (S)-3-((2-((S)-amino((1r,4S)-4-methylcyclohexyl)methyl)imidazo[1,2-b]pyridazin-6-yl)methyl)piperidin-2-one (40.0 mg, 112 μmol, 61% yield), was obtained as a light-yellow solid and used directly in the next step.

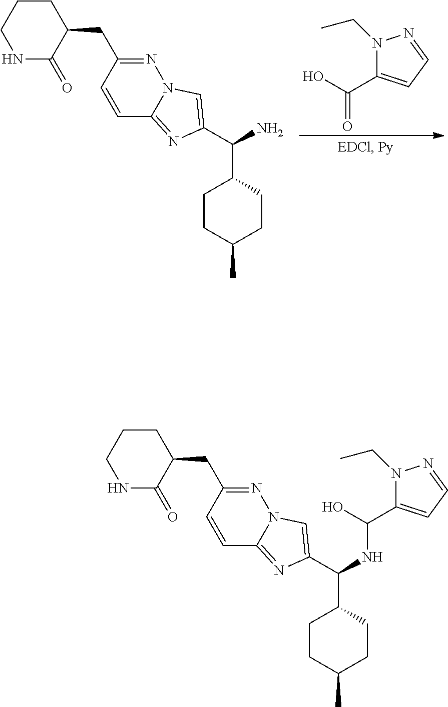

The title compound was synthesized following General Procedure 4, using (S)-3-((2-((S)-amino((1r,4S)-4-methylcyclohexyl)methyl)imidazo[1,2-b]pyridazin-6-yl)methyl)piperidin-2-one (40.0 mg, 112 μmol, 1.00 eq), 1-ethyl-1H-pyrazole-5-carboxylic acid (23.6 mg, 169 μmol, 1.50 eq), EDCI (64.7 mg, 338 μmol, 3.00 eq), and pyridine (2.00 mL). The reaction mixture was stirred for 1 h. The residue was purified by prep-HPLC (Phenomenex C18 75×30 mm×3 um; mobile phase: [water (FA)-ACN]; B %: 30%-60%, 7 min). The product, 1-ethyl-N—((S)-((1r,4S)-4-methylcyclohexyl)(6-(((S)-2-oxopiperidin-3-yl)methyl)imidazo[1,2-b]pyridazin-2-yl)methyl)-1H-pyrazole-5-carboxamide (Compound 39, 32.07 mg, 65.87 μmol, 59% yield), was obtained as a yellow solid. LCMS [M+H]⁺=478.2 m/z.

### Example 51: Preparation of Compound 40

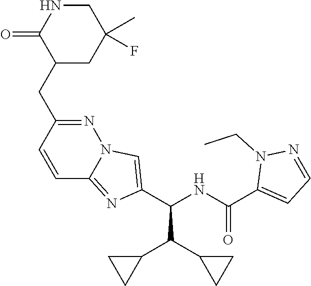 [SMILES: CCn1nccc1C(=O)N[C@H](c1cn2nc(CC3CC(C)(F)CNC3=O)ccc2n1)C(C1CC1)C1CC1]

### N-((1S)-2,2-dicyclopropyl-1-(6-((5-fluoro-5-methyl-2-oxopiperidin-3-yl)methyl)imidazo[1,2-b]pyridazin-2-yl)ethyl)-1-ethyl-1H-pyrazole-5-carboxamide. (Compound 40)

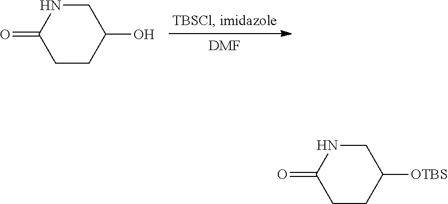

To a solution of 5-hydroxypiperidin-2-one (1.00 g, 8.69 mmol, 1.00 eq) in DMF (10.0 mL) was added TBSCl (1.96 g, 13.0 mmol, 1.60 mL, 1.50 eq) and imidazole (1.18 g, 17.4 mmol, 2.00 eq), then the reaction mixture was stirred at RT for 2 h. The reaction mixture was diluted with H₂O (90.0 mL) and extracted with DCM (150 mL×3). The combined organic phases were dried over Na₂SO₄ and concentrated to give a residue. The product, 5-((tert-butyldimethylsilyl)oxy)piperidin-2-one (2.55 g, crude), was obtained as a white solid. LCMS [M+H]⁺=230.0 m/z.

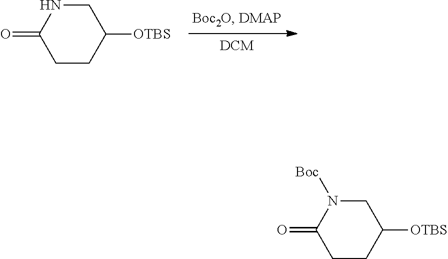

To a solution of 5-((tert-butyldimethylsilyl)oxy)piperidin-2-one (2.50 g, 10.9 mmol, 1.00 eq) in DCM (20.0 mL) was added DMAP (666 mg, 5.45 mmol, 0.500 eq) and Boc₂O (4.76 g, 21.8 mmol, 5.01 mL, 2.00 eq). The reaction mixture was stirred at RT for 2 h. The reaction mixture was concentrated under the vacuum to give a residue. The residue was purified by column chromatography (PE:EtOAc=3:1, R_{f}=0.50, SiO₂, PE:EtOAc=1:0 to 3:1). The product, tert-butyl 5-((tert-butyldimethylsilyl)oxy)-2-oxopiperidine-1-carboxylate (2.10 g, 6.37 mmol, 58% yield), was obtained as a light yellow oil. LCMS [M−99]⁺=230.4 m/z. ¹H NMR (400 MHz, CDCl₃) δ 4.16 (br t, J=4.8 Hz, 1H), 3.65 (t, J=4.8 Hz, 2H), 2.78-2.65 (m, 1H), 2.47-2.40 (m, 17.2 Hz, 1H), 2.02-1.91 (m, 1H), 1.88-1.81 (m 1H), 1.53 (s, 9H), 0.89 (s, 9H), 0.09 (s, 6H).

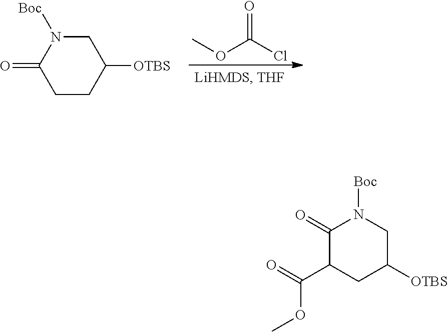

To a solution of tert-butyl 5-((tert-butyldimethylsilyl)oxy)-2-oxopiperidine-1-carboxylate (500 mg, 1.52 mmol, 1.00 eq) in THE (5.00 mL) was added dropwise LiHMDS (1.00M, 3.19 mL, 2.10 eq) at −70° C. The reaction mixture was stirred at −70° C. for 1 h, then methyl chloroformate (690 mg, 7.30 mmol, 566 μL, 4.81 eq) was added dropwise at −70° C. The reaction mixture was further stirred at −70° C. for 1 h. The reaction solution was quenched with 200 mL saturated NH₄Cl solution, then diluted with H₂O 100 mL and extracted with EtOAc (250 mL×3). The combined organic layers were dried over Na₂SO₄, filtered, and concentrated under reduced pressure to give a residue. The product, 1-(tert-butyl) 3-methyl 5-((tert-butyldimethylsilyl)oxy)-2-oxopiperidine-1,3-dicarboxylate (540 mg, 1.39 mmol, 92% yield), was obtained as a light-yellow oil. LCMS [M−99]⁺=287.9 m/z.

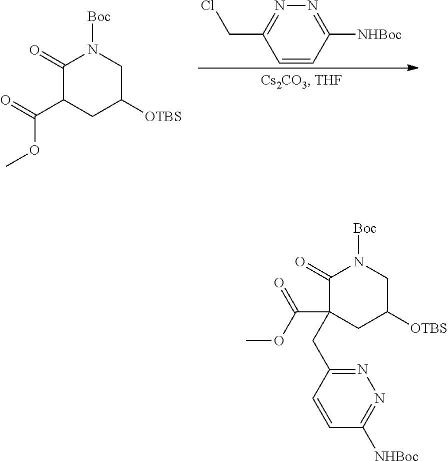

A solution of 1-(tert-butyl) 3-methyl 5-((tert-butyldimethylsilyl)oxy)-2-oxopiperidine-1,3-dicarboxylate (530 mg, 1.37 mmol, 1.00 eq) and tert-butyl (6-(chloromethyl)pyridazin-3-yl)carbamate (333 mg, 1.37 mmol, 1.00 eq) in THF (8.00 mL) was added Cs₂CO₃ (891 mg, 2.74 mmol, 2.00 eq). The reaction mixture was stirred at 55° C. for 1 h. The reaction mixture was diluted with H₂O (100 mL) and extracted with DCM (100 mL×3). The combined organic layers were dried over Na₂SO₄, filtered, and concentrated under reduced pressure to give a residue. The residue was purified by prep-TLC (PE:EtOAc=3:1, R_{f}=0.30). The product, 1-(tert-butyl) 3-methyl 3-((6-((tert-butoxy carbonyl)amino)pyridazin-3-yl)methyl)-5-((tert-butyldimethylsilyl)oxy)-2-oxopiperidine-1,3-dicarboxylate (550 mg, 925 μmol, 68% yield), was obtained as a white solid. LCMS [M+H]⁺=595.6 m/z. ¹H NMR (400 MHz, CDCl₃) δ 8.12 (dd, J₁=9.2 Hz, J₂=4.8 Hz, 1H), 7.62 (br s, 1H), 7.46 (dd, J₁=11.8 Hz, J₂=9.2 Hz, 1H), 4.26-4.16 (m, 0.5H), 4.05-3.95 (m, 0.5H), 3.76 (d, J=4.8 Hz, 3H), 3.70-3.50 (m, 4H), 2.57 (dd, J₁=14.0 Hz, J₂=5.6 Hz, 0.5H), 2.42 (dd, J₁=12.0 Hz, J₂=5.6 Hz, 1H), 1.98 (dd, J₁=14.0 Hz, J₂=8.0 Hz, 0.5H), 1.54 (s, 9H), 1.52 (d, J=1.8 Hz, 9H), 0.85 (d, J=4.8 Hz, 9H), 0.05 (d, J=2.0 Hz, 6H).

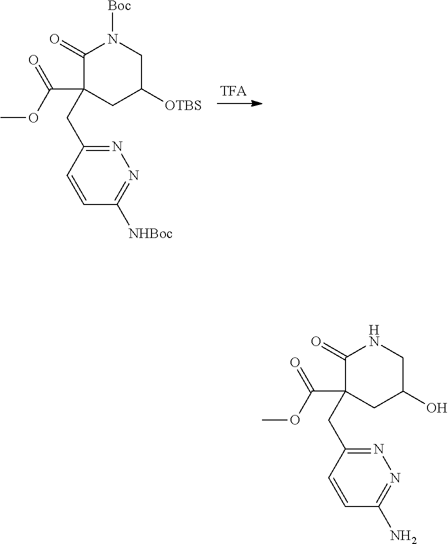 [SMILES: COC(=O)C1(Cc2ccc(NC(=O)OC(C)(C)C)nn2)CC(O[Si](C)(C)C(C)(C)C)CN(C(=O)OC(C)(C)C)C1=O ; COC(=O)C1(Cc2ccc(N)nn2)CC(O)CNC1=O]

A solution of 1-(tert-butyl) 3-methyl 3-((6-((tert-butoxycarbonyl)amino)pyridazin-3-yl)methyl)-5-((tert-butyldimethylsilyl)oxy)-2-oxopiperidine-1,3-dicarboxylate (530 mg, 891 μmol, 1.00 eq) in TFA (5.00 mL) was stirred at RT for 1 h. The reaction mixture was concentrated under the vacuum to give a residue. Methyl 3-((6-aminopyridazin-3-yl)methyl)-5-hydroxy-2-oxopiperidine-3-carboxylate (350 mg, 888 μmol, quantitative, TFA salt) was obtained as a yellow oil. LCMS [M+H]⁺=303.0 m/z.

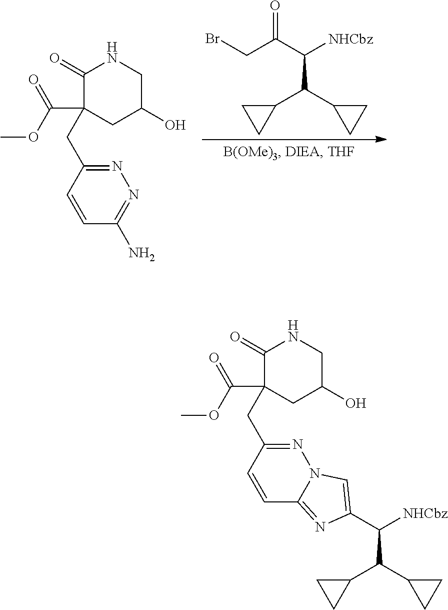 [SMILES: COC(=O)C1(Cc2ccc(N)nn2)CC(O)CNC1=O ; O=C(N[C@H](C(=O)CBr)C(C1CC1)C1CC1)OCc1ccccc1 ; COC(=O)C1(Cc2ccc3nc([C@@H](NC(=O)OCc4ccccc4)C(C4CC4)C4CC4)cn3n2)CC(O)CNC1=O]

Methyl 3-((2-((S)-1-(((benzyloxy)carbonyl)amino)-2,2-dicyclopropylethyl)imidazo[1,2-b]pyridazin-6-yl)methyl)-5-hydroxy-2-oxopiperidine-3-carboxylate was synthesized following General Procedure 1, using methyl 3-((6-aminopyridazin-3-yl)methyl)-5-hydroxy-2-oxopiperidine-3-carboxylate (350 mg, 888 μmol, 1.00 eq, TFA salt), Intermediate 1 (371 mg, 976 μmol, 1.10 eq), B(OMe)₃ (553 mg, 5.33 mmol, 602 μL, 6.00 eq), and DIEA (688 mg, 5.33 mmol, 928 μL, 6.00 eq). After 3 h at 70° C. and extraction with 10% MeOH in DCM, crude product was purified by prep-TLC (PE:EtOAc=0:1, R_{f}=0.30) to afford methyl 3-((2-((S)-1-(((benzyloxy)carbonyl)amino)-2,2-dicyclopropylethyl)imidazo[1,2-b]pyridazin-6-yl)methyl)-5-hydroxy-2-oxopiperidine-3-carboxylate (140 mg, 249 μmol, 28% yield) as a yellow oil. LCMS [M+H]⁺=562.5 m/z.

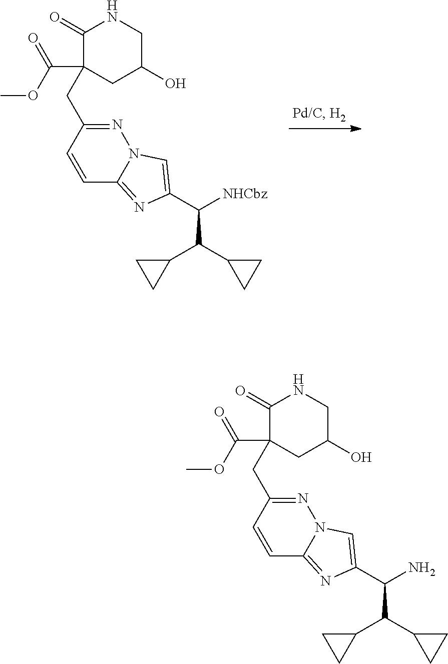 [SMILES: COC(=O)C1(Cc2ccc3nc([C@@H](NC(=O)OCc4ccccc4)C(C4CC4)C4CC4)cn3n2)CC(O)CNC1=O ; COC(=O)C1(Cc2ccc3nc([C@@H](N)C(C4CC4)C4CC4)cn3n2)CC(O)CNC1=O]

Methyl 3-((2-((S)-1-amino-2,2-dicyclopropylethyl)imidazo[1,2-b]pyridazin-6-yl)methyl)-5-hydroxy-2-oxopiperidine-3-carboxylate was synthesized following General Procedure 2, using methyl 3-((2-((S)-1-(((benzyloxy)carbonyl)amino)-2,2-dicyclopropylethyl)imidazo[1,2-b]pyridazin-6-yl)methyl)-5-hydroxy-2-oxopiperidine-3-carboxylate (130 mg, 231 μmol, 1.00 eq), TMSI (232 mg, 1.16 mmol, 158 μL, 5.00 eq), and DCM (2.00 mL) and letting stir at RT for 2 h and using 5:1 DCM/MeOH for extraction. The product, methyl 3-((2-((S)-1-amino-2,2-dicyclopropylethyl)imidazo[1,2-b]pyridazin-6-yl)methyl)-5-hydroxy-2-oxopiperidine-3-carboxylate (100 mg, crude), was obtained as a yellow oil. LCMS [M+H]⁺=428.2 m/z.

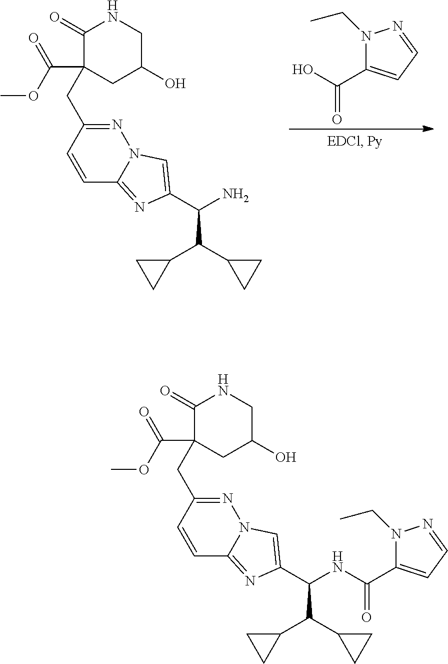

Methyl 3-((2-((S)-2,2-dicyclopropyl-1-(1-ethyl-1H-pyrazole-5-carboxamido)ethyl)imidazo[1,2-b]pyridazin-6-yl)methyl)-5-hydroxy-2-oxopiperidine-3-carboxylate was synthesized following General Procedure 4, using methyl 3-((2-((S)-1-amino-2,2-dicyclopropylethyl)imidazo[1,2-b]pyridazin-6-yl)methyl)-5-hydroxy-2-oxopiperidine-3-carboxylate (90 mg, crude), 1-ethyl-1H-pyrazole-5-carboxylic acid (88.5 mg, 632 μmol, 3.00 eq), EDCI (80.7 mg, 421 μmol, 2.00 eq), and pyridine (2.00 mL). The reaction mixture was stirred for 1 h. After reaction workup (5:1 DCM/MeOH), the residue was purified by prep-TLC (DCM:MeOH=10:1, R_{f}=0.50). The product, methyl 3-((2-((S)-2,2-dicyclopropyl-1-(1-ethyl-1H-pyrazole-5-carboxamido)ethyl)imidazo[1,2-b]pyridazin-6-yl)methyl)-5-hydroxy-2-oxopiperidine-3-carboxylate (70.0 mg, 127 μmol, 61% yield over 2 steps), was obtained as a yellow oil. LCMS [M+H]⁺=550.5 m/z.

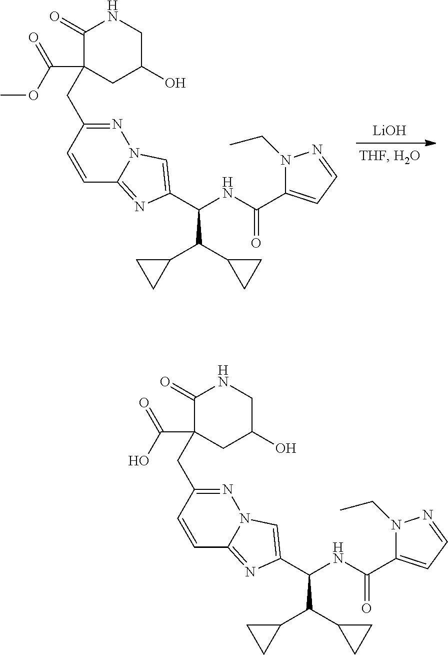 [SMILES: CCn1nccc1C(=O)N[C@H](c1cn2nc(CC3(C(=O)OC)CC(O)CNC3=O)ccc2n1)C(C1CC1)C1CC1 ; "LiOH" ; CCn1nccc1C(=O)N[C@H](c1cn2nc(CC3(C(=O)O)CC(O)CNC3=O)ccc2n1)C(C1CC1)C1CC1]

To a solution of methyl 3-((2-((S)-2,2-dicyclopropyl-1-(1-ethyl-1H-pyrazole-5-carboxamido)ethyl)imidazo[1,2-b]pyridazin-6-yl)methyl)-5-hydroxy-2-oxopiperidine-3-carboxylate (65.0 mg, 118 μmol, 1.00 eq) in THF (2.00 mL) was added LiOH·H₂O (19.9 mg 473 μmol, 4.00 eq) in H₂O (0.200 mL). The reaction mixture was stirred at RT for 1 h. The reaction mixture was adjusted to pH=4 with 1 M HCl, then the reaction mixture was concentrated under vacuum to give a residue. The crude product, 3-((2-((S)-2,2-dicyclopropyl-1-(1-ethyl-1H-pyrazole-5-carboxamido)ethyl)imidazo[1,2-b]pyridazin-6-yl)methyl)-5-hydroxy-2-oxopiperidine-3-carboxylic acid (63.0 mg, crude), was obtained as a yellow oil. LCMS [M+H]⁺=536.3 m/z.

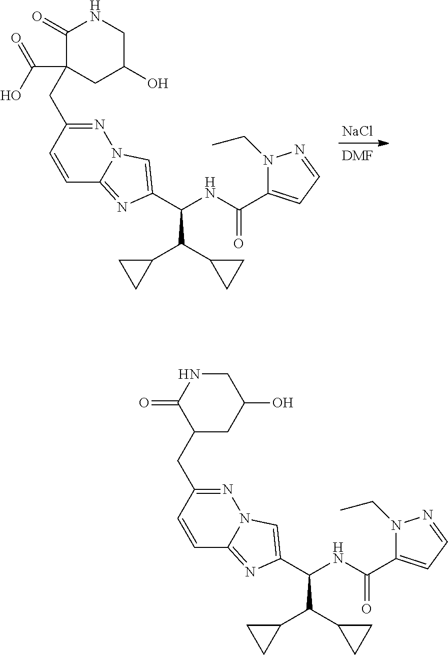

To a solution of crude 3-((2-((S)-2,2-dicyclopropyl-1-(1-ethyl-1H-pyrazole-5-carboxamido)ethyl)imidazo[1,2-b]pyridazin-6-yl)methyl)-5-hydroxy-2-oxopiperidine-3-carboxylic acid (63.0 mg, 118 μmol, 1.00 eq) in DMF (1.00 mL) was added NaCl (13.8 mg, 235 μmol, 2.00 eq), the reaction mixture was stirred at 120° C. for 30 min. The reaction mixture was diluted with H₂O (50.0 mL) and extracted with a mixture of 10:1 DCM/MeOH (50.0 mL×3). The combined organic phases were dried over Na₂SO₄, filtered, and concentrated to give a residue. The residue was purified by prep-TLC (DCM:MeOH=10:1, R_{f}=0.50). The product, N-((1S)-2,2-dicyclopropyl-1-(6-((5-hydroxy-2-oxopiperidin-3-yl)methyl)imidazo[1,2-b]pyridazin-2-yl)ethyl)-1-ethyl-1H-pyrazole-5-carboxamide (2.05 mg, 4.18 μmol, 3.5% yield over 2 steps), was obtained as a yellow oil. LCMS [M+H]⁺=492.6 m/z.

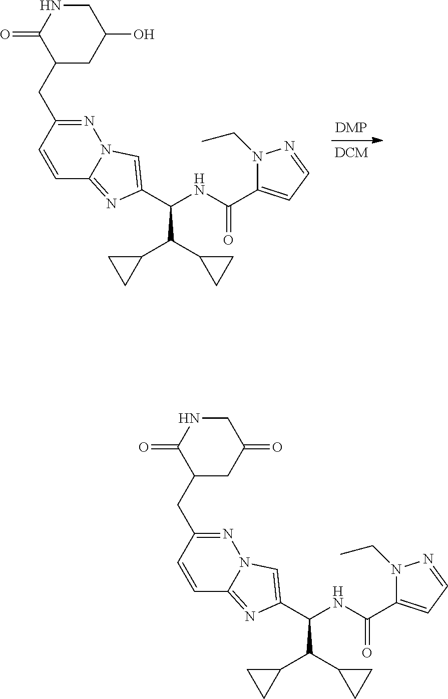

To a solution of N-((1S)-2,2-dicyclopropyl-1-(6-((5-hydroxy-2-oxopiperidin-3-yl)methyl)imidazo[1,2-b]pyridazin-2-yl)ethyl)-1-ethyl-1H-pyrazole-5-carboxamide (50.0 mg, 101.7 μmol, 1.00 eq) in DCM (1.00 mL) was added DMP (129 mg, 305 μmol, 94.5 μL, 3.00 eq). The reaction mixture was stirred at RT for 2 h. The reaction was diluted with H₂O (30.0 mL) and extracted with DCM (30.0 mL×3), dried with anhydrous Na₂SO₄, filtered, and concentrated under vacuum. The residue was purified by prep-TLC (DCM:MeOH=10:1, R_{f}=0.60). The product, N-((1S)-2,2-dicyclopropyl-1-(6-((2,5-dioxopiperidin-3-yl)methyl)imidazo[1,2-b]pyridazin-2-yl)ethyl)-1-ethyl-1H-pyrazole-5-carboxamide (40.0 mg, 81.7 μmol, 80% yield), was obtained as a yellow oil. LCMS [M+H]⁺=490.2 m/z. ¹H NMR (400 MHz, CDCl₃) δ 7.88 (s, 1H), 7.84 (d, J=9.2 Hz, 1H), 7.49 (d, J=2.0 Hz, 1H), 7.42 (br d, J=7.6 Hz, 1H), 7.03 (dd, J₁=9.6 Hz, J₂=2.4 Hz, 1H), 6.65 (t, J=1.8 Hz, 1H), 5.85 (br s, 1H), 5.55 (dd, J₁=8.4 Hz, J₂=5.6 Hz, 1H), 4.66-4.55 (m, 2H), 4.11-4.03 (m, 1H), 3.95-3.86 (m, 1H), 3.58-3.51 (m, 1H), 3.38-3.28 (m, 1H), 3.00 (dd, J₁=15.2 Hz, J₂=7.8 Hz, 1H), 2.92-2.83 (m, 1H), 2.64 (dd, J₁=16.8 Hz, J₂=12.8 Hz, 1H), 1.44 (t, J=7.2 Hz, 3H), 0.98-0.91 (m, 1H), 0.77-0.62 (m, 2H), 0.58-0.26 (m, 6H), 0.22-0.17 (m, 1H), 0.16-0.11 (m, 1H).

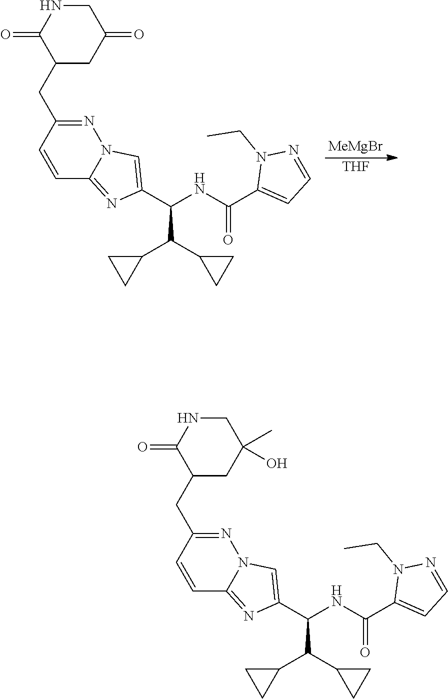 [SMILES: "MeMgBr" ; CCn1nccc1C(=O)N[C@H](c1cn2nc(CC3CC(=O)CNC3=O)ccc2n1)C(C1CC1)C1CC1 ; CCn1nccc1C(=O)N[C@H](c1cn2nc(CC3CC(C)(O)CNC3=O)ccc2n1)C(C1CC1)C1CC1 ; "THF"]

To a solution of N-((1S)-2,2-dicyclopropyl-1-(6-((2,5-dioxopiperidin-3-yl)methyl)imidazo[1,2-b]pyridazin-2-yl)ethyl)-1-ethyl-1H-pyrazole-5-carboxamide (40.0 mg, 81.7 μmol, 1.00 eq) in THF (1.00 mL) was added MeMgBr (3.00 M, 272 μL, 10.0 eq) at 0° C., then the reaction mixture was stirred at RT for 1 h. The reaction solution was quenched with 50.0 mL saturated NH₄Cl solution. The reaction mixture was diluted with H₂O (50.0 mL) and extracted with a mixture of DCM and MeOH (DCM:MeOH=10:1, 100 mL×3). The combined organic phases were dried over Na₂SO₄ and concentrated to give a residue. The residue was purified by prep-TLC (DCM:MeOH=10:1, R_{f}=0.60). The product, N-((1S)-2,2-dicyclopropyl-1-(6-((5-hydroxy-5-methyl-2-oxopiperidin-3-yl)methyl)imidazo[1,2-b]pyridazin-2-yl)ethyl)-1-ethyl-1H-pyrazole-5-carboxamide (30.0 mg, 59.3 μmol, 73% yield), was obtained as a yellow oil. LCMS [M+H]⁺=506.2 m/z. ¹H NMR (400 MHz, CDCl₃) δ 7.87 (s, 1H), 7.79 (br d, J=9.2 Hz, 1H), 7.50-7.40 (m, 2H), 6.99 (dd, J₁=9.2 Hz, J₂=2.4 Hz, 1H), 6.65 (d, J=2.0 Hz, 1H), 6.17-5.95 (m, 1H), 5.53 (dd, J₁=8.4 Hz, J₂=5.6 Hz, 1H), 5.30 (s, 1H), 4.59 (q, J=7.2 Hz, 2H), 3.49-2.88 (m, 6H), 2.62 (s, 1H), 1.43 (t, J=7.2 Hz, 3H), 1.38 (s, 3H), 0.96-0.91 (m, 1H), 0.76-0.62 (m, 2H), 0.56-0.38 (m, 4H), 0.34-0.29 (m, 2H), 0.22-0.14 (m, 1H), 0.12-0.11 (m, 1H).

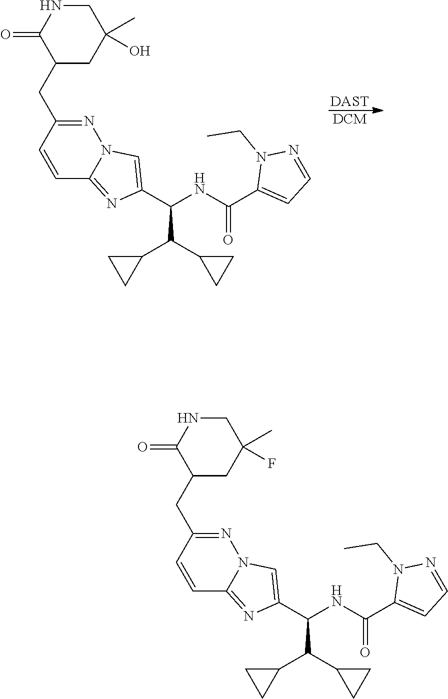

To a solution of N-((1S)-2,2-dicyclopropyl-1-(6-((5-hydroxy-5-methyl-2-oxopiperidin-3-yl)methyl)imidazo[1,2-b]pyridazin-2-yl)ethyl)-1-ethyl-1H-pyrazole-5-carboxamide (30.0 mg, 59.3 μmol, 1.00 eq) in DCM (1.00 mL) was added DAST (57.4 mg, 356 μmol, 47.0 μL, 6.00 eq) at 0° C. The reaction mixture was stirred at RT for 1 h. The reaction solution was quenched with 50.0 mL saturated NaHCO₃ solution. The residue was diluted with H₂O (50.0 mL) and extracted with 10:1 DCM/MeOH (100 mL×3). The combined organic phases were dried over Na₂SO₄ and concentrated to give a residue. The residue was purified by prep-TLC (DCM:MeOH=10:1, R_{f}=0.60), then further purified by prep-SFC (DAICEL CHIRALPAK AD (250 mm×30 mm, 10 μm); 35% mobile phase: [0.1% NH₃H₂O EtOH]). Compound 40, N-((1S)-2,2-dicyclopropyl-1-(6-((5-fluoro-5-methyl-2-oxopiperidin-3-yl)methyl)imidazo[1,2-b]pyridazin-2-yl)ethyl)-1-ethyl-1H-pyrazole-5-carboxamide (7.38 mg, 13.7 μmol, 23% yield), was obtained as a mixture of at least two stereoisomers, isolated as an off-white solid. LCMS [M+H]⁺=508.2 m/z.

### Example 52: Preparation of Compound 41

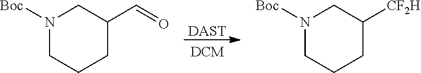

To a solution of tert-butyl 3-formylpiperidine-1-carboxylate (3.00 g, 14.0 mmol, 1.00 eq) in DCM (30.0 mL) was added DAST (11.3 g, 70.3 mmol, 9.29 mL, 5.00 eq) in DCM (30.0 mL) at 0° C. The reaction mixture was stirred at RT for 1 h. The reaction mixture was quenched by saturated aqueous NaHCO₃ (30.0 mL) at 0° C., and then diluted with H₂O (30.0 mL) and extracted with EtOAc (30.0 mL×3). The combined organic layers were washed with saturated aqueous NaHCO₃ (30.0 mL×2), dried over Na₂SO₄, filtered, and concentrated under reduced pressure to give a residue. The residue was purified by column chromatography (SiO₂, PE/EtOAc=100/1 to 10/1) (Plate 2, PE/EtOAc=3:1). The product, tert-butyl 3-(difluoromethyl)piperidine-1-carboxylate (1.40 g, 5.95 mmol, 42% yield), was obtained as a white solid. LCMS [M−55]⁺=180.4 m/z.

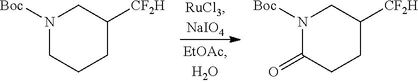

To a solution of tert-butyl 3-(difluoromethyl)piperidine-1-carboxylate (1.00 g, 4.25 mmol, 1.00 eq) in EtOAc (10.0 mL) and H₂O (2.00 mL) was added NaIO₄ (4.55 g, 21.2 mmol, 1.18 mL, 5.00 eq) and RuCl₃ (440 mg, 2.13 mmol, 141 μL, 0.50 eq) at 0° C. The reaction mixture was stirred at RT for 1 h. The reaction mixture was quenched by addition of saturated aqueous Na₂S₂O₃ (10.0 mL) at 0° C., then diluted with H₂O (30.0 mL) and extracted with EtOAc (30.0 mL×3). The combined organic layers were dried over Na₂SO₄, filtered, and concentrated under reduced pressure to give a residue. The residue was purified by column chromatography (SiO₂, PE/EtOAc=100/1 to 10/1) (PE:EtOAc=3:1). The product, tert-butyl 5-(difluoromethyl)-2-oxopiperidine-1-carboxylate (600 mg, 2.41 mmol, 57% yield), was obtained as a white solid. LCMS [M+Na]⁺=272.1 m/z.

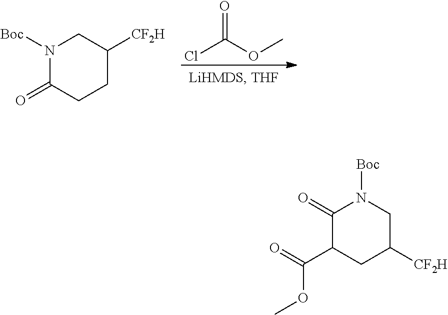

To a solution of tert-butyl 5-(difluoromethyl)-2-oxopiperidine-1-carboxylate (600 mg, 2.41 mmol, 1.00 eq) in THE (5.00 mL) was added LiHMDS (1 M, 3.61 mL, 1.50 eq) at −70° C. The reaction mixture was stirred at −70° C. for 30 mins. Methyl chloroformate (341 mg, 3.61 mmol, 279 μL, 1.50 eq) was then added at −70° C. The reaction mixture was further stirred at −70° C. for 1 h. The reaction mixture was quenched by addition of saturated aqueous NH₄Cl (10.0 mL at 0° C.), and then diluted with H₂O (30.0 mL) and extracted with EtOAc (30.0 mL×3). The combined organic layers were washed with saturated aqueous NaHCO₃ (30.0 mL×2), dried over Na₂SO₄, filtered, and concentrated under reduced pressure to give a residue. The residue was purified by column chromatography (SiO₂, PE/EtOAc=100/1 to 10/1) (PE:EtOAc=3:1). The product, 1-(tert-butyl) 3-methyl 5-(difluoromethyl)-2-oxopiperidine-1,3-dicarboxylate (550 mg 1.79 mmol, 74% yield), was obtained as a white solid. LCMS [M−99]⁺=208.1 m/z.

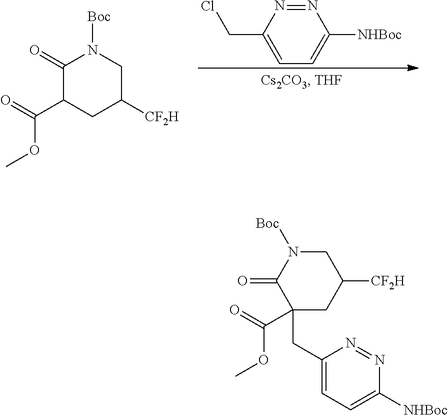

To a solution of 1-(tert-butyl) 3-methyl 5-(difluoromethyl)-2-oxopiperidine-1,3-dicarboxylate (250 mg, 813 μmol, 1.00 eq) in THF (5.00 mL) was added tert-butyl (6-(chloromethyl)pyridazin-3-yl)carbamate (237 mg, 976 μmol, 1.20 eq) and Cs₂CO₃ (795 mg, 2.44 mmol, 3.00 eq). The reaction mixture was stirred at 70° C. for 1 h. The reaction mixture was diluted with H₂O (30.0 mL) and extracted with EtOAc (30.0 mL×3). The combined organic layers were dried over Na₂SO₄, filtered, and concentrated under reduced pressure to give a residue. The residue was purified by prep-TLC (SiO₂, PE:EtOAc=2:1) (PE:EtOAc=2:1). The product, 1-(tert-butyl) 3-methyl 3-((6-((tert-butoxycarbonyl)amino)pyridazin-3-yl)methyl)-5-(difluoromethyl)-2-oxopiperidine-1,3-dicarboxylate (270 mg, 524 μmol, 65% yield), was obtained as a white solid that was used directly in the next step.

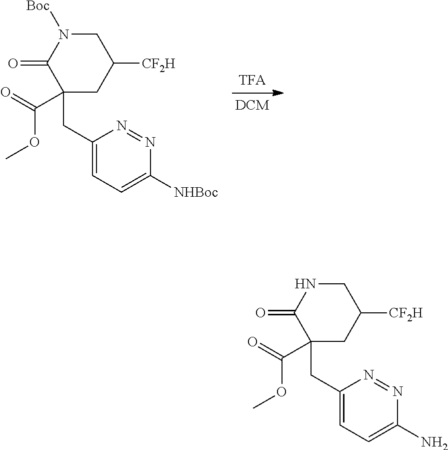 [SMILES: COC(=O)C1(Cc2ccc(NC(=O)OC(C)(C)C)nn2)CC(C(F)F)CN(C(=O)OC(C)(C)C)C1=O ; COC(=O)C1(Cc2ccc(N)nn2)CC(C(F)F)CNC1=O]

To a solution of 1-(tert-butyl) 3-methyl 3-((6-((tert-butoxycarbonyl)amino)pyridazin-3-yl)methyl)-5-(difluoromethyl)-2-oxopiperidine-1,3-dicarboxylate (270 mg, 524 μmol, 1.00 eq) in DCM (1.00 mL) was added TFA (1.66 g, 14.5 mmol, 1.08 mL, 27.8 eq) at 0° C. The reaction mixture was stirred at RT for 12 h. The reaction mixture was concentrated under reduced pressure to give a residue. Methyl 3-((6-aminopyridazin-3-yl)methyl)-5-(difluoromethyl)-2-oxopiperidine-3-carboxylate (220 mg, 513 μmol, 98% yield, TFA salt) was obtained as an orange oil. LCMS [M+H]⁺=315.1 m/z.

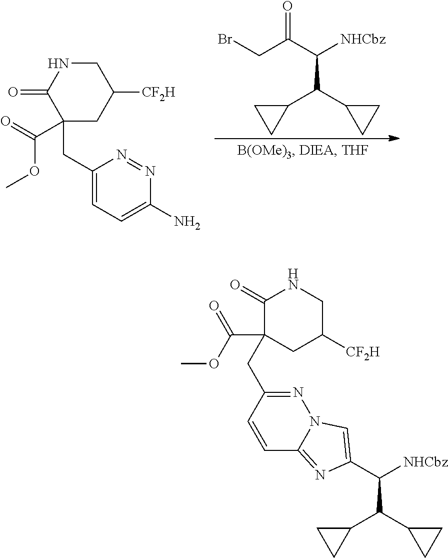 [SMILES: COC(=O)C1(Cc2ccc(N)nn2)CC(C(F)F)CNC1=O ; O=C(N[C@H](C(=O)CBr)C(C1CC1)C1CC1)OCc1ccccc1 ; COC(=O)C1(Cc2ccc3nc([C@@H](NC(=O)OCc4ccccc4)C(C4CC4)C4CC4)cn3n2)CC(C(F)F)CNC1=O]

Methyl 3-((2-((S)-1-(((benzyloxy)carbonyl)amino)-2,2-dicyclopropylethyl)imidazo[1,2-b]pyridazin-6-yl)methyl)-5-(difluoromethyl)-2-oxopiperidine-3-carboxylate was synthesized following General Procedure 1, using methyl 3-((6-aminopyridazin-3-yl)methyl)-5-(difluoromethyl)-2-oxopiperidine-3-carboxylate (135 mg, 315 μmol, 1.00 eq, TFA salt), Intermediate 1 (143 mg, 378 μmol, 1.20 eq), B(OMe)₃ (163 mg, 1.58 mmol, 178 μL, 5.00 eq), and DIEA (203 mg, 1.58 mmol, 274 μL, 5.00 eq). After 1 h at 70° C. and washing with saturated aqueous NH₄Cl during workup, crude product was purified by prep-TLC (SiO₂, DCM:MeOH=10:1). The product, methyl 3-((2-((S)-1-(((benzyloxy)carbonyl)amino)-2,2-dicyclopropylethyl)imidazo[1,2-b]pyridazin-6-yl)methyl)-5-(difluoromethyl)-2-oxopiperidine-3-carboxylate (181 mg, 303 μmol, 96% yield), was obtained as a yellow oil. LCMS [M+H]⁺=596.4 m/z.

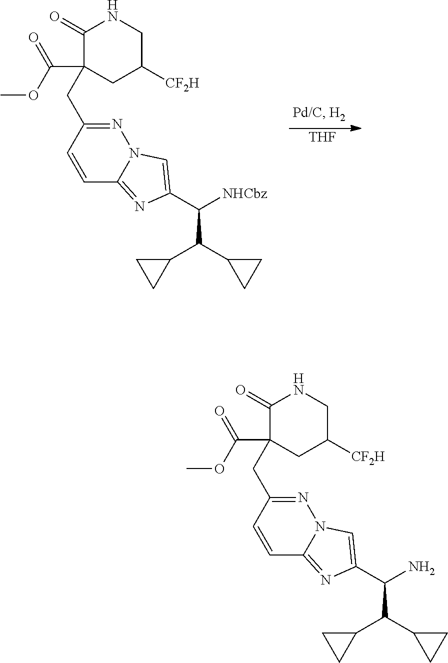 [SMILES: COC(=O)C1(Cc2ccc3nc([C@@H](NC(=O)OCc4ccccc4)C(C4CC4)C4CC4)cn3n2)CC(C(F)F)CNC1=O ; COC(=O)C1(Cc2ccc3nc([C@@H](N)C(C4CC4)C4CC4)cn3n2)CC(C(F)F)CNC1=O ; "THF"]

To a solution of methyl 3-((2-((S)-1-(((benzyloxy)carbonyl)amino)-2,2-dicyclopropylethyl)imidazo[1,2-b]pyridazin-6-yl)methyl)-5-(difluoromethyl)-2-oxopiperidine-3-carboxylate (181 mg, 303 μmol, 1.00 eq) in THF (5.00 mL) was added Pd/C (20.0 mg, 10.0% purity) under N₂ atmosphere. The suspension was degassed and purged with H₂ 3 times. The reaction mixture was stirred under H₂ (15 psi) at RT for 2 h. The reaction mixture was filtered, and the filter cake was washed with 100 mL MeOH. The filtrate was concentrated under reduced pressure to give a residue. Methyl 3-((2-((S)-1-amino-2,2-dicyclopropylethyl)imidazo[1,2-b]pyridazin-6-yl)methyl)-5-(difluoromethyl)-2-oxopiperidine-3-carboxylate (140 mg, 303 μmol, quantitative) was obtained as a white solid. LCMS [M+H]⁺=462.2 m/z.

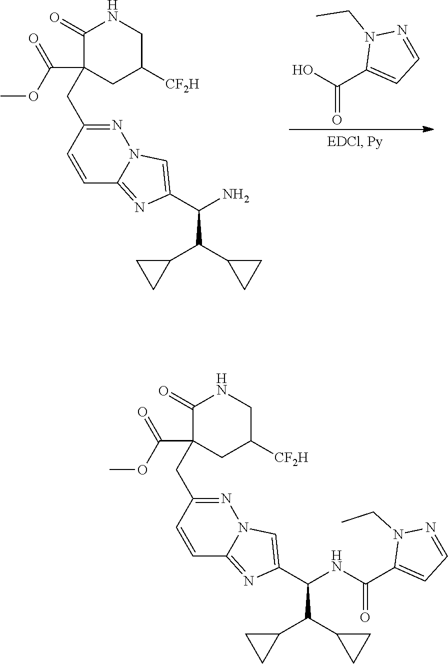

To a solution of methyl 3-((2-((S)-1-amino-2,2-dicyclopropylethyl)imidazo[1,2-b]pyridazin-6-yl)methyl)-5-(difluoromethyl)-2-oxopiperidine-3-carboxylate (65.0 mg, 140 μmol, 1.00 eq) in pyridine (5.00 mL) was added 1-ethyl-1H-pyrazole-5-carboxylic acid (23.6 mg, 169 μmol, 1.20 eq) and EDCI (81.0 mg, 422 μmol, 3.00 eq). The reaction mixture was stirred at RT for 1 h. The reaction mixture was diluted with H₂O (30.0 mL) and extracted with EtOAc (30.0 mL×3). The combined organic layers were washed with citric acid solution (30.0 mL×2), dried over Na₂SO₄, filtered, and concentrated under reduced pressure to give a residue. The residue was purified by prep-TLC (SiO₂, DCM:MeOH=10:1). Methyl 3-((2-((S)-2,2-dicyclopropyl-1-(1-ethyl-1H-pyrazole-5-carboxamido)ethyl)imidazo[1,2-b]pyridazin-6-yl)methyl)-5-(difluoromethyl)-2-oxopiperidine-3-carboxylate (40.0 mg, 68.5 μmol, 49% yield) was obtained as an orange oil. LCMS [M+H]⁺=584.4 m/z.

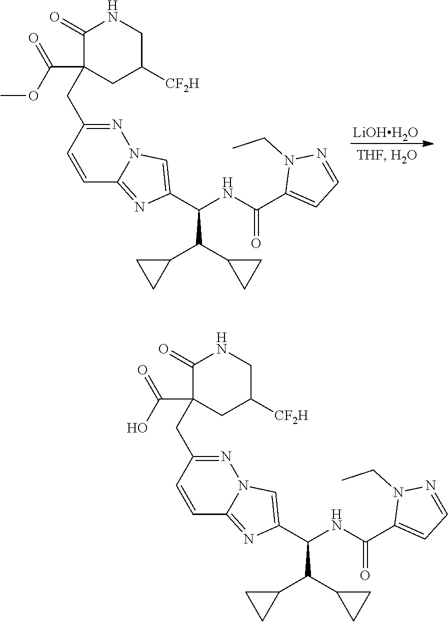

To a solution of methyl 3-((2-((S)-2,2-dicyclopropyl-1-(1-ethyl-1H-pyrazole-5-carboxamido)ethyl)imidazo[1,2-b]pyridazin-6-yl)methyl)-5-(difluoromethyl)-2-oxopiperidine-3-carboxylate (40.0 mg, 68.5 μmol, 1.00 eq) in THF (3.00 mL) was added LiOH·H₂O (8.63 mg, 205 μmol, 3.00 eq) and H₂O (1.00 mL) at 0° C. The reaction mixture was stirred at RT for 1 h. The reaction mixture was diluted with H₂O (10.0 mL) and adjusted to pH=3 with 1 M HCl. The reaction mixture was concentrated under reduced pressure to give a residue. The product, 3-((2-((S)-2,2-dicyclopropyl-1-(1-ethyl-1H-pyrazole-5-carboxamido)ethyl)imidazo[1,2-b]pyridazin-6-yl)methyl)-5-(difluoromethyl)-2-oxopiperidine-3-carboxylic acid (35.0 mg, crude), was obtained as a colorless solid. LCMS [M+H]⁺=570.5 m/z.

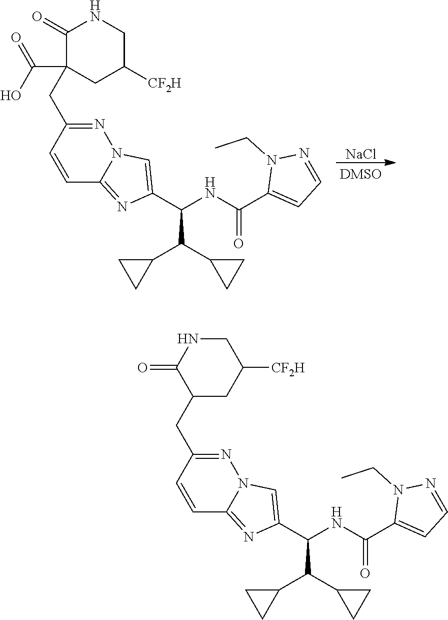

To a solution of 3-((2-((S)-2,2-dicyclopropyl-1-(1-ethyl-1H-pyrazole-5-carboxamido)ethyl)imidazo[1,2-b]pyridazin-6-yl)methyl)-5-(difluoromethyl)-2-oxopiperidine-3-carboxylic acid (35.0 mg, crude) in DMSO (5.00 mL) was added NaCl (17.9 mg, 307 μmol, 5.00 eq). The reaction mixture was stirred at 100° C. for 2 h. The reaction mixture was diluted with H₂O (30.0 mL) and extracted with EtOAc (30.0 mL×3). The combined organic layers were washed with H₂O (30.0 mL×2), dried over Na₂SO₄, filtered, and concentrated under reduced pressure to give a residue. The residue was purified by prep-TLC (SiO₂, DCM:MeOH=10:1). The product was obtained as a mixture of stereoisomers, N-((1S)-2,2-dicyclopropyl-1-(6-((5-(difluoromethyl)-2-oxopiperidin-3-yl)methyl)imidazo[1,2-b]pyridazin-2-yl)ethyl)-1-ethyl-1H-pyrazole-5-carboxamide (Compound 41, 28.5 mg, 53.2 μmol, 78% yield over 2 steps), isolated as an orange solid. LCMS [M+H]⁺=526.2 m/z.

### Example 53: Preparation of Compounds 42 and 43

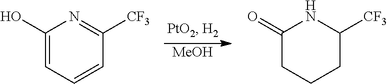 [SMILES: "MeOH" ; Oc1cccc(C(F)(F)F)n1 ; O=C1CCCC(C(F)(F)F)N1]

To a solution of 6-(trifluoromethyl)pyridin-2-ol (15.0 g, 91.9 mmol, 1.00 eq) in MeOH (100 mL) was added PtO₂ (2.09 g, 9.20 mmol, 0.100 eq). The reaction mixture was stirred at RT for 16 h under H₂. The reaction mixture was filtered and concentrated under reduced pressure to give a residue. The product, 6-(trifluoromethyl)piperidin-2-one (14.0 g, 83.7 mmol, 91% yield), was obtained as a white solid. ¹H NMR (400 MHz, CDCl₃) δ 6.90-6.66 (m, 1H), 4.03-3.85 (m, 1H), 2.42-2.33 (m, 2H), 2.12-1.96 (m, 2H), 1.91-1.69 (m, 2H).

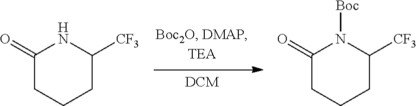 [SMILES: O=C1CCCC(C(F)(F)F)N1 ; CC(C)(C)OC(=O)N1C(=O)CCCC1C(F)(F)F]

To a solution of 6-(trifluoromethyl)piperidin-2-one (14.0 g, 83.7 mmol, 1.00 eq) in DCM (140 mL) was added (Boc)₂O (21.9 g, 100 mmol, 23.0 mL, 1.20 eq), TEA (16.9 g, 167 mmol, 23.3 mL, 2.00 eq) and DMAP (5.12 g, 41.88 mmol, 0.500 eq). The reaction mixture was stirred at RT for 5 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The product, tert-butyl 2-oxo-6-(trifluoromethyl)piperidine-1-carboxylate (18.0 g, crude), was obtained as a white solid and used in the next step without further purification.

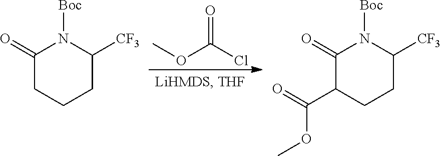

To a solution of tert-butyl 2-oxo-6-(trifluoromethyl)piperidine-1-carboxylate (17.0 g, crude) in THE (170 mL) was added LiHMDS (1.00 M, 69.9 mL, 1.10 eq) and was stirred at −70° C. for 30 min. The reaction mixture was added methyl chloroformate (9.62 g, 101 mmol, 7.89 mL, 1.60 eq). The reaction mixture was further stirred at −70° C. for 1 h. The reaction mixture was diluted with H₂O (100 mL) and extracted with DCM (100 mL×3). The combined organic layers were washed with brine (100 mL×3), dried over Na₂SO₄, filtered, and concentrated under reduced pressure to give a residue. The residue was purified by column chromatography (SiO₂, PE:EtOAc=3:1, monitoring, PE:EtOAc=3:1, Rf (P1)=0.15). The product, 1-(tert-butyl) 3-methyl 2-oxo-6-(trifluoromethyl)piperidine-1,3-dicarboxylate (19.0 g, crude), was obtained as a white solid as was used directly in the next step without further purification.

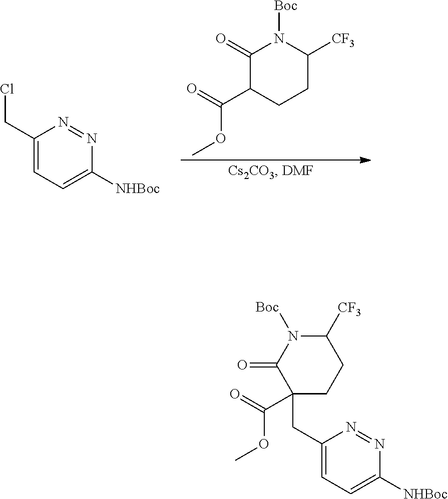

To a solution of tert-butyl (6-(chloromethyl)pyridazin-3-yl)carbamate (1.00 g, 4.10 mmol, 1.00 eq) and 1-(tert-butyl) 3-methyl 2-oxo-6-(trifluoromethyl)piperidine-1,3-dicarboxylate (1.47 g, crude, 4.51 mmol, 1.10 eq) in DMF (3.00 mL) was added Cs₂CO₃ (4.01 g, 12.3 mmol, 3.00 eq). The reaction mixture was stirred at RT for 3 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The product, 1-(tert-butyl) 3-methyl 3-((6-((tert-butoxycarbonyl)amino)pyridazin-3-yl)methyl)-2-oxo-6-(trifluoromethyl)piperidine-1,3-dicarboxylate (2.00 g, crude), was obtained as a white solid. LCMS [M+H]⁺=533.3 m/z.

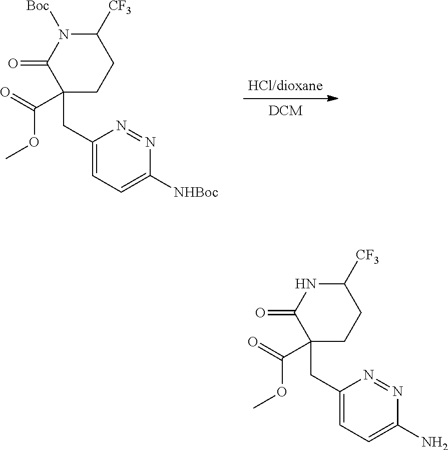 [SMILES: COC(=O)C1(Cc2ccc(NC(=O)OC(C)(C)C)nn2)CCC(C(F)(F)F)N(C(=O)OC(C)(C)C)C1=O ; COC(=O)C1(Cc2ccc(N)nn2)CCC(C(F)(F)F)NC1=O]

To a solution of 1-(tert-butyl) 3-methyl 3-((6-((tert-butoxycarbonyl)amino)pyridazin-3-yl)methyl)-2-oxo-6-(trifluoromethyl)piperidine-1,3-dicarboxylate (2.00 g, crude) in DCM (3.00 mL) was added HCl/dioxane (4.00 M, 22.2 mL, 23.6 eq). The reaction mixture was stirred at RT for 1 h. The reaction mixture was concentrated under reduced pressure to give a residue. The product, methyl 3-((6-aminopyridazin-3-yl)methyl)-2-oxo-6-(trifluoromethyl)piperidine-3-carboxylate (1.25 g, crude, HCl salt), was obtained as a white solid. LCMS [M+H]⁺=333.1 m/z.

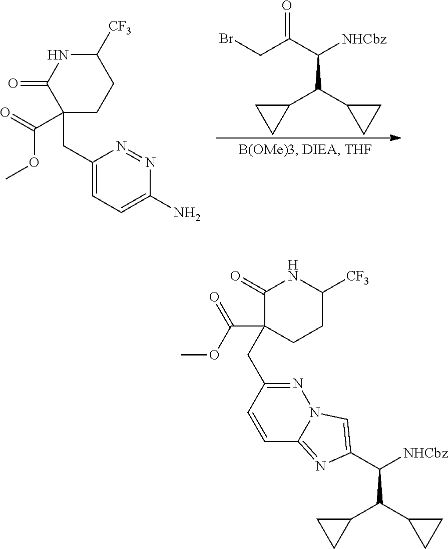 [SMILES: COC(=O)C1(Cc2ccc(N)nn2)CCC(C(F)(F)F)NC1=O ; O=C(N[C@H](C(=O)CBr)C(C1CC1)C1CC1)OCc1ccccc1 ; COC(=O)C1(Cc2ccc3nc([C@@H](NC(=O)OCc4ccccc4)C(C4CC4)C4CC4)cn3n2)CCC(C(F)(F)F)NC1=O]

Methyl 3-((2-((S)-1-(((benzyloxy)carbonyl)amino)-2,2-dicyclopropylethyl)imidazo[1,2-b]pyridazin-6-yl)methyl)-2-oxo-6-(trifluoromethyl)piperidine-3-carboxylate was synthesized following General Procedure 1, using methyl 3-((6-aminopyridazin-3-yl)methyl)-2-oxo-6-(trifluoromethyl)piperidine-3-carboxylate (0.650 g, crude), Intermediate 1 (743 mg, 1.96 mmol, 1.00 eq), B(OMe)₃ (1.02 g, 9.78 mmol, 1.10 mL, 5.00 eq), and DIEA (1.26 g, 9.78 mmol, 1.70 mL, 5.00 eq). The reaction mixture was stirred for 2 hat 70° C. The product, methyl 3-((2-((S)-1-(((benzyloxy)carbonyl)amino)-2,2-dicyclopropylethyl)imidazo[1,2-b]pyridazin-6-yl)methyl)-2-oxo-6-(trifluoromethyl)piperidine-3-carboxylate (1.00 g, crude), was obtained as a yellow oil. LCMS [M+H]⁺=614.1 m/z.

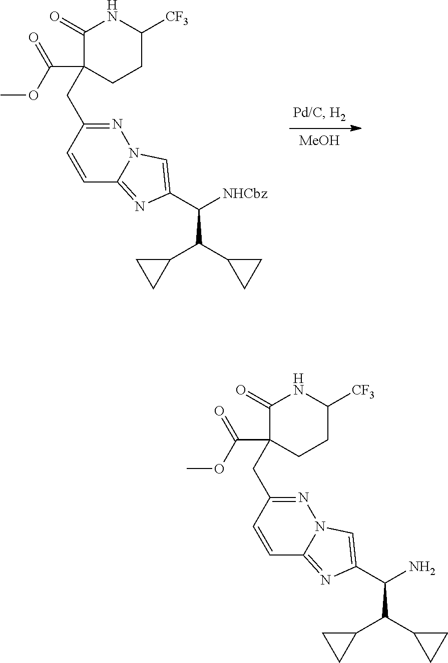 [SMILES: "MeOH" ; COC(=O)C1(Cc2ccc3nc([C@@H](NC(=O)OCc4ccccc4)C(C4CC4)C4CC4)cn3n2)CCC(C(F)(F)F)NC1=O ; COC(=O)C1(Cc2ccc3nc([C@@H](N)C(C4CC4)C4CC4)cn3n2)CCC(C(F)(F)F)NC1=O]

To a solution of methyl 3-((2-((S)-1-(((benzyloxy)carbonyl)amino)-2,2-dicyclopropylethyl)imidazo[1,2-b]pyridazin-6-yl)methyl)-2-oxo-6-(trifluoromethyl)piperidine-3-carboxylate (1.00 g, crude) in MeOH (5.00 mL) was added Pd/C (100 mg, 1.63 mmol, 10% purity, 1.00 eq). The reaction mixture was stirred at RT for 3 h under H₂ (15 psi). The reaction mixture was filtered and concentrated under reduced pressure to give a residue. Crude methyl 3-((2-((S)-1-amino-2,2-dicyclopropylethyl)imidazo[1,2-b]pyridazin-6-yl)methyl)-2-oxo-6-(trifluoromethyl)piperidine-3-carboxylate was obtained as a white solid (781 mg, crude) and used directly in the next step. LCMS [M+H]⁺=480.2 m/z.

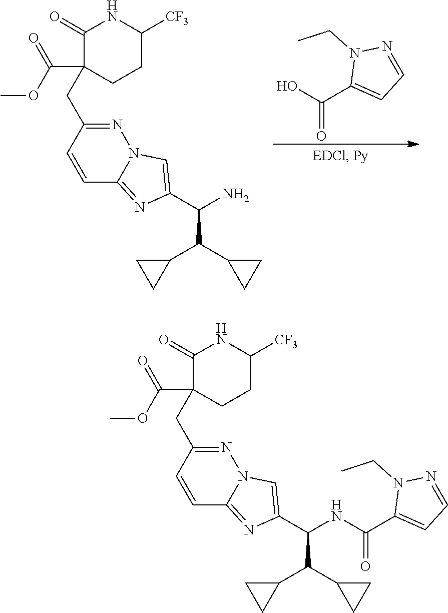

To a solution of methyl 3-((2-((S)-1-amino-2,2-dicyclopropylethyl)imidazo[1,2-b]pyridazin-6-yl)methyl)-2-oxo-6-(trifluoromethyl)piperidine-3-carboxylate (270 mg, crude) and 1-ethyl-1H-pyrazole-5-carboxylic acid (94.6 mg, 675 μmol, 1.20 eq) in pyridine (10.0 mL) was added EDCI (323 mg, 1.69 mmol, 3.00 eq). The reaction mixture was stirred at RT for 1 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The crude product was purified by prep-HPLC (Phenomenex luna C18 150×25 mm×10 μm; mobile phase: [water (FA)-ACN]; B %: 36%-66%, 10 min). Methyl 3-((2-((S)-2,2-dicyclopropyl-1-(1-ethyl-1H-pyrazole-5-carboxamido)ethyl)imidazo[1,2-b]pyridazin-6-yl)methyl)-2-oxo-6-(trifluoromethyl)piperidine-3-carboxylate (300 mg, 499 μmol, 68% yield over 5 steps) was obtained as a white solid. ¹H NMR (400 MHz, CDCl₃) δ 7.88-7.73 (m, 2H), 7.49 (d, J=2.0 Hz, 1H), 7.37 (d, J=8.0 Hz, 1H), 7.10-6.96 (m, 1H), 6.63 (d, J=2.0 Hz, 1H), 6.04-5.86 (m, 1H), 5.61-5.49 (m, 1H), 4.69-4.49 (m, 2H), 4.03-3.88 (m, 1H), 3.85-3.81 (m, 3H), 3.75-3.67 (m, 1H), 3.44-3.32 (m, 1H), 2.39-2.29 (m, 1H), 2.15-2.02 (m, 3H), 1.44 (t, J=8.0 Hz, 3H), 1.00-0.90 (m, 1H), 0.76-0.61 (m, 2H), 0.57-0.40 (m, 4H), 0.37-0.29 (m, 2H), 0.22-0.06 (m, 2H).

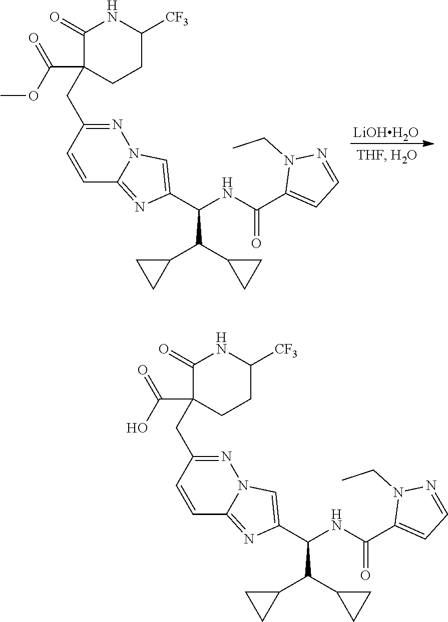

To a solution of methyl 3-((2-((S)-2,2-dicyclopropyl-1-(1-ethyl-1H-pyrazole-5-carboxamido)ethyl)imidazo[1,2-b]pyridazin-6-yl)methyl)-2-oxo-6-(trifluoromethyl)piperidine-3-carboxylate (200 mg, 332 μmol, 1.00 eq) in THF (3.00 mL) and H₂O (1.00 mL) was added LiOH·H₂O (139 mg, 3.32 mmol, 10.0 eq). The reaction mixture was stirred at RT for 3 h. The combined reaction mixture was washed with EtOAc (20.0 mL×2). The pH of the aqueous phase was adjusted to 3 with 1 M HCl and extracted with EtOAc (20.0 mL×2). The combined organic layers were dried over Na₂SO₄, filtered, and concentrated under reduced pressure to give a residue. The product, 3-((2-((S)-2,2-dicyclopropyl-1-(1-ethyl-1H-pyrazole-5-carboxamido)ethyl)imidazo[1,2-b]pyridazin-6-yl)methyl)-2-oxo-6-(trifluoromethyl)piperidine-3-carboxylic acid (193 mg, crude), was obtained as a white solid and used directly in the next step without further purification.

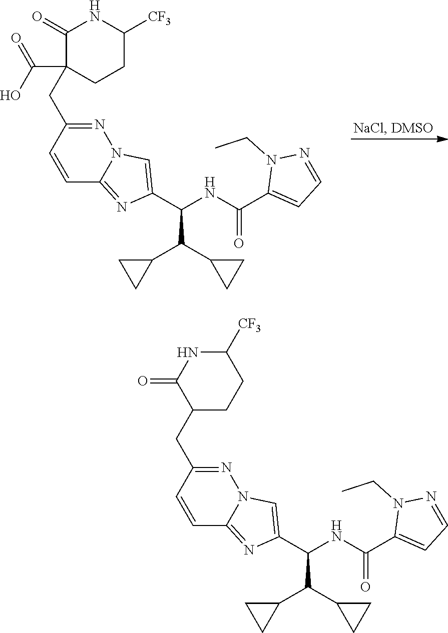

To a solution of 3-((2-((S)-2,2-dicyclopropyl-1-(1-ethyl-1H-pyrazole-5-carboxamido)ethyl)imidazo[1,2-b]pyridazin-6-yl)methyl)-2-oxo-6-(trifluoromethyl)piperidine-3-carboxylic acid (0.193 g, crude) in DMSO (5.00 mL) was added NaCl (191 mg, 3.28 mmol, 10.0 eq). The reaction mixture was stirred at 130° C. for 1 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The crude product was purified by prep-SFC (DAICEL CHIRAL PAK AD (250 mm×30 mm, 10 μm); 45% mobile phase: [0.1% NH₃H₂O EtOH]) and prep-HPLC (Phenomenexluna C18 150×25 mm×10 μm; mobile phase: [water(FA)-ACN]; B %: 33%-63%, 10 min). The first eluting, single stereoisomer of N-((1S)-2,2-dicyclopropyl-1-(6-((2-oxo-6-(trifluoromethyl)piperidin-3-yl)methyl)imidazo[1,2-b]pyridazin-2-yl)ethyl)-1-ethyl-1H-pyrazole-5-carboxamide (Compound 42, 7.96 mg, 14.4 μmol, 4.3% yield over 2 steps) was obtained as a yellow solid. LCMS [M+H]⁺=544.3 m/z. The fourth eluting, single stereoisomer of N-((1S)-2,2-dicyclopropyl-1-(6-((2-oxo-6-(trifluoromethyl)piperidin-3-yl)methyl)imidazo[1,2-b]pyridazin-2-yl)ethyl)-1-ethyl-1H-pyrazole-5-carboxamide (Compound 43, 16.8 mg, 30.0 μmol, 9.0% yield over 2 steps) was obtained as a yellow solid. LCMS [M+H]⁺=544.2 m/z.

### Example 54: Preparation of Compounds 44, 45, and 46

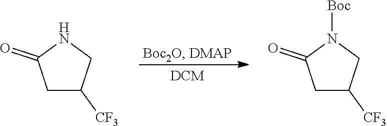

To a solution of 4-(trifluoromethyl)pyrrolidin-2-one (0.800 g, 5.23 mmol, 1.00 eq) in DCM (20.0 mL) was added TEA (1.06 g, 10.4 mmol, 1.45 mL, 2.00 eq), DMAP (192 mg, 1.57 mmol, 0.300 eq) and Boc₂O (1.48 g, 6.79 mmol, 1.56 mL, 1.30 eq). The reaction mixture was stirred at RT for 2 h. The reaction mixture was concentrated under reduced pressure to give a residue. The residue was purified by column chromatography (SiO₂, PE/EtOAc=30/1 to 3/1) to afford tert-butyl 2-oxo-4-(trifluoromethyl)pyrrolidine-1-carboxylate (1.15 g, 4.54 mmol, 87% yield) as a white solid. ¹H NMR (400 MHz, CDCl₃) δ 3.97-3.80 (m, 2H), 3.08-3.04 (m, 1H), 2.82-2.71 (m, 2H), 1.54 (s, 9H).

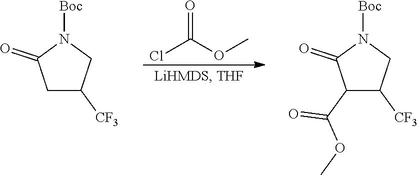

To a solution of tert-butyl 2-oxo-4-(trifluoromethyl)pyrrolidine-1-carboxylate (1.55 g, 6.12 mmol, 1.00 eq) in THF (25.0 mL) was added dropwise LiHMDS (1.00 M, 12.0 mL, 1.96 eq) at −70° C. After addition, the reaction mixture was stirred at −70° C. for 30 min, then methyl chloroformate (2.47 g, 26.1 mmol, 2.02 mL, 4.27 eq) was added dropwise at −70° C. under N₂. The resulting mixture was stirred at −70° C. for 2 h under N₂. The reaction mixture was quenched with saturated aqueous NH₄Cl (50.0 mL) at 0° C., and the resulting mixture was stirred for an additional 30 min, then extracted with EtOAc (50.0 mL×2). The combined organic layers were washed with saturated aqueous NaHCO₃ (50.0 mL×2) and brine (50.0 mL), then dried over Na₂SO₄, filtered, and concentrated under reduced pressure to afford 1-(tert-butyl) 3-methyl 2-oxo-4-(trifluoromethyl)pyrrolidine-1,3-dicarboxylate (1.80 g, 5.78 mmol, 94% yield) as a yellow oil. ¹H NMR (400 MHz, CDCl₃) δ 4.07-4.02 (m, 1H), 3.84 (s, 3H), 3.80-3.71 (m, 2H), 3.60-3.52 (m, 1H), 1.53 (s, 9H).

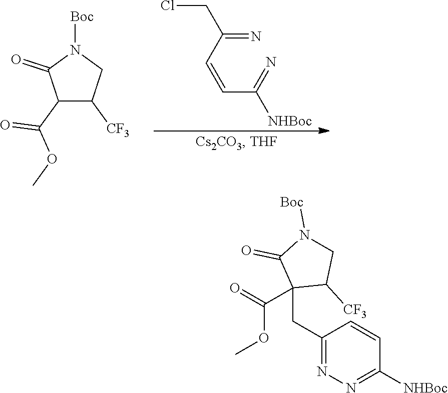

To a solution of 1-(tert-butyl) 3-methyl 2-oxo-4-(trifluoromethyl)pyrrolidine-1,3-dicarboxylate (0.900 g, 2.89 mmol, 1.00 eq) and tert-butyl (6-(chloromethyl)pyridazin-3-yl)carbamate (0.900 g, 3.69 mmol, 1.28 eq) in THF (20.0 mL) was added Cs₂CO₃ (10.0 g, 30.69 mmol, 10.6 eq). The reaction mixture was stirred at 55° C. for 4 h. The reaction mixture was filtered with EtOAc (200 mL), and the filtrate was concentrated to give a residue, which was purified by reverse-phase HPLC (0.1% FA condition) to afford 1-(tert-butyl) 3-methyl 3-((6-((tert-butoxycarbonyl)amino)pyridazin-3-yl)methyl)-2-oxo-4-(trifluoromethyl)pyrrolidine-1,3-dicarboxylate (0.700 g, 1.35 mmol, 47% yield) as a green gum. LCMS [M+H]⁺=519.4 m/z.

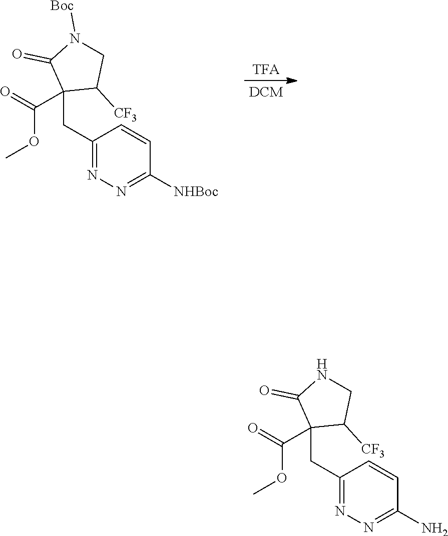 [SMILES: COC(=O)C1(Cc2ccc(NC(=O)OC(C)(C)C)nn2)C(=O)N(C(=O)OC(C)(C)C)CC1C(F)(F)F ; COC(=O)C1(Cc2ccc(N)nn2)C(=O)NCC1C(F)(F)F]

To a solution of 1-(tert-butyl) 3-methyl 3-((6-((tert-butoxycarbonyl)amino)pyridazin-3-yl)methyl)-2-oxo-4-(trifluoromethyl)pyrrolidine-1,3-dicarboxylate (0.700 g, 1.35 mmol, 1.00 eq) in DCM (6.00 mL) was added TFA (3.08 g, 27.0 mmol, 2 mL, 20.0 eq) at 0° C. The reaction mixture was stirred at RT for 3 h. The reaction mixture was concentrated under reduced pressure to afford methyl 3-((6-aminopyridazin-3-yl)methyl)-2-oxo-4-(trifluoromethyl)pyrrolidine-3-carboxylate (0.600 g, crude, TFA salt) as a green oil. LCMS [M+H]⁺=319.2 m/z.

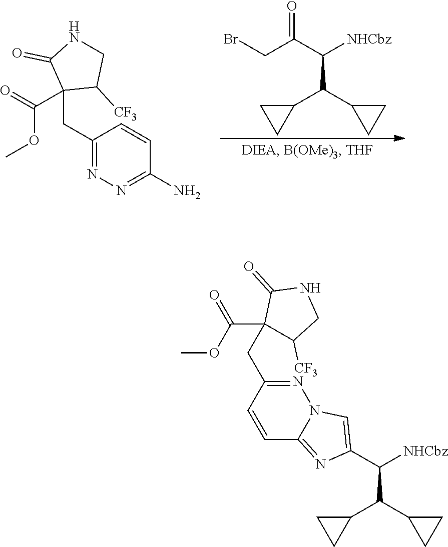 [SMILES: COC(=O)C1(Cc2ccc(N)nn2)C(=O)NCC1C(F)(F)F ; O=C(N[C@H](C(=O)CBr)C(C1CC1)C1CC1)OCc1ccccc1 ; COC(=O)C1(Cc2ccc3nc([C@@H](NC(=O)OCc4ccccc4)C(C4CC4)C4CC4)cn3n2)C(=O)NCC1C(F)(F)F]

To a solution of methyl 3-((6-aminopyridazin-3-yl)methyl)-2-oxo-4-(trifluoromethyl)pyrrolidine-3-carboxylate (0.500 g, crude, TFA salt) and Intermediate 1 (439 mg, 1.16 mmol, 1.00 eq) in THF (15.0 mL) was added DIEA (1.49 g, 11.5 mmol, 2.01 mL, 10.0 eq), and B(OMe)₃ (601 mg, 5.78 mmol, 653 μL, 5.00 eq). The reaction mixture was stirred at 70° C. for 3 h under N₂. The reaction mixture was diluted with H₂O (30.0 mL) and extracted with EtOAc (30.0 mL×3). The combined organic layers were washed with water (40.0 mL) followed by brine (40.0 mL), dried over Na₂SO₄, filtered, and concentrated under reduced pressure to give a residue. The residue was purified by column chromatography (SiO₂, PE/EtOAc=5/1 to 1/1) to afford methyl 3-((2-((S)-1-(((benzyloxy)carbonyl)amino)-2,2-dicyclopropylethyl)imidazo[1,2-b]pyridazin-6-yl)methyl)-2-oxo-4-(trifluoromethyl)pyrrolidine-3-carboxylate (0.490 g, 817 μmol, 73% yield over 2 steps) as a yellow solid. LCMS [M+H]⁺=600.4 m/z.

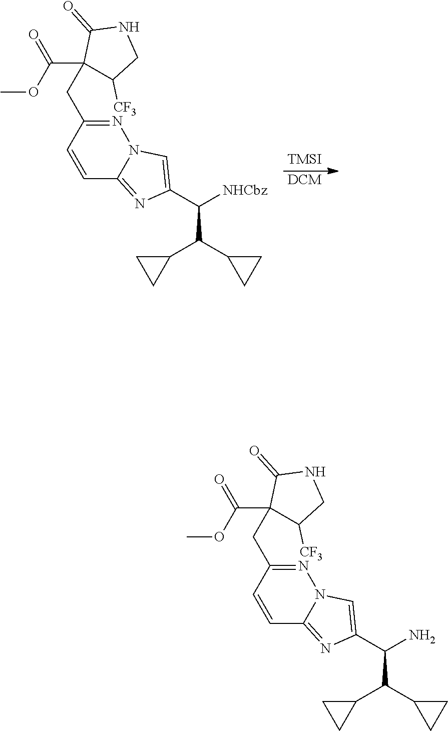 [SMILES: "TMSI" ; COC(=O)C1(Cc2ccc3nc([C@@H](NC(=O)OCc4ccccc4)C(C4CC4)C4CC4)cn3n2)C(=O)NCC1C(F)(F)F ; COC(=O)C1(Cc2ccc3nc([C@@H](N)C(C4CC4)C4CC4)cn3n2)C(=O)NCC1C(F)(F)F]

To a solution of methyl 3-((2-((S)-1-(((benzyloxy)carbonyl)amino)-2,2-dicyclopropylethyl)imidazo[1,2-b]pyridazin-6-yl)methyl)-2-oxo-4-(trifluoromethyl)pyrrolidine-3-carboxylate (300 mg, 500 umol, 1.00 eq) in DCM (8.00 mL) was added TMSI (200 mg, 1.00 mmol, 136 μL, 2.00 eq) at 0° C. The reaction mixture was stirred at RT for 2 h. The residue was diluted with water (20.0 mL) and adjusted with HCl (1 M) to pH=3, then washed with EtOAc (30.0 mL). The aqueous phase was adjusted to pH ˜9 with saturated aqueous NaHCO₃ and extracted with EtOAc (30.0 mL×2). The combined organic layers were dried over anhydrous Na₂SO₄, filtered, and concentrated under reduced pressure to afford methyl 3-((2-((S)-1-amino-2,2-dicyclopropylethyl)imidazo[1,2-b]pyridazin-6-yl)methyl)-2-oxo-4-(trifluoromethyl)pyrrolidine-3-carboxylate (0.190 g, 408 μmol, 82% yield) as a yellow solid. LCMS [M+H]⁺=466.2 m/z.

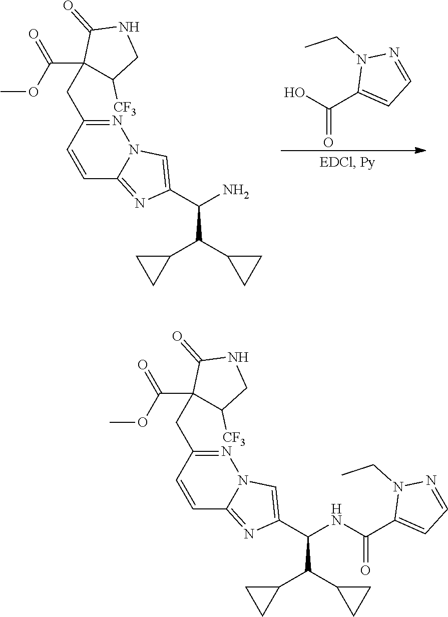

To a solution of methyl 3-((2-((S)-1-amino-2,2-dicyclopropylethyl)imidazo[1,2-b]pyridazin-6-yl)methyl)-2-oxo-4-(trifluoromethyl)pyrrolidine-3-carboxylate (0.190 g, 408 μmol, 1.00 eq) and 1-ethyl-1H-pyrazole-5-carboxylic acid (68.6 mg, 489 μmol, 1.20 eq) in pyridine (3.00 mL) was added EDCI (235 mg, 1.22 mmol, 3.00 eq). The reaction mixture was stirred at RT for 2 h. The reaction mixture was diluted with H₂O (30.0 mL) and extracted with EtOAc (30.0 mL×2). The combined organic layers were washed with H₂O (40.0 mL×2), dried over Na₂SO₄, filtered, and concentrated under reduced pressure to give a residue. The residue combined with a second reaction's crude product and was purified by prep-TLC (SiO₂, PE/EtOAc=0:1) to afford methyl 3-((2-((S)-2,2-dicyclopropyl-1-(1-ethyl-1H-pyrazole-5-carboxamido)ethyl)imidazo[1,2-b]pyridazin-6-yl)methyl)-2-oxo-4-(trifluoromethyl)pyrrolidine-3-carboxylate (275 mg, 468 μmol) as a yellow solid. LCMS [M+H]⁺=588.4 m/z.

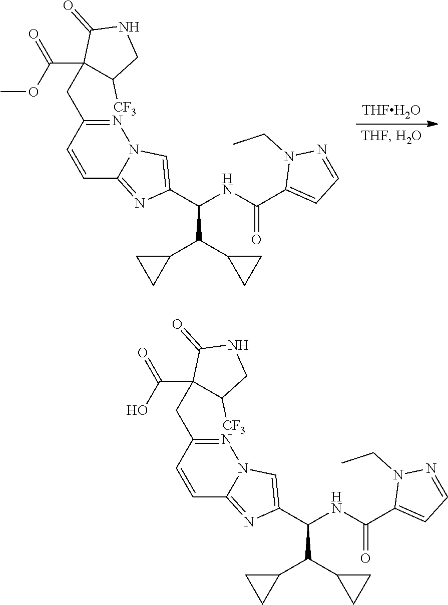

To a solution of methyl 3-((2-((S)-2,2-dicyclopropyl-1-(1-ethyl-1H-pyrazole-5-carboxamido)ethyl)imidazo[1,2-b]pyridazin-6-yl)methyl)-2-oxo-4-(trifluoromethyl)pyrrolidine-3-carboxylate (225 mg, 382 μmol, 1.00 eq) in THF (10.0 mL) was added a solution of LiOH·H₂O (64.2 mg, 1.53 mmol, 4.00 eq) in H₂O (3.00 mL) at 0° C. The reaction mixture was stirred at RT for 16 h. The reaction mixture was diluted with H₂O (30.0 mL), adjusted with 1 M HCl to pH=3, then extracted with EtOAc (30.0 mL×2). The combined organic layers were dried over anhydrous Na₂SO₄, filtered, and concentrated under reduced pressure to afford 3-((2-((S)-2,2-dicyclopropyl-1-(1-ethyl-1H-pyrazole-5-carboxamido)ethyl)imidazo[1,2-b]pyridazin-6-yl)methyl)-2-oxo-4-(trifluoromethyl)pyrrolidine-3-carboxylic acid (0.200 g, 348 μmol, 91% yield) as a yellow solid. LCMS [M+H]⁺=574.3 m/z.

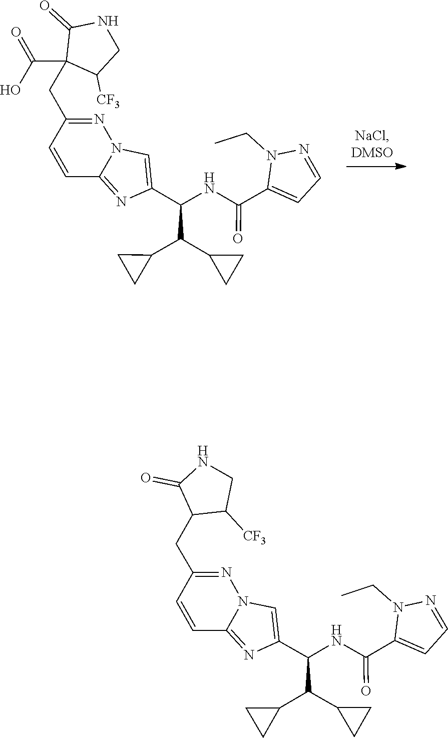

To a solution of 3-((2-((S)-2,2-dicyclopropyl-1-(1-ethyl-1H-pyrazole-5-carboxamido)ethyl)imidazo[1,2-b]pyridazin-6-yl)methyl)-2-oxo-4-(trifluoromethyl)pyrrolidine-3-carboxylic acid (0.200 g, 348 μmol, 1.00 eq) in DMSO (2.50 mL) was added NaCl (40.7 mg 697 μmol, 2.00 eq). The reaction mixture was stirred at 110° C. for 1 h. The reaction mixture was filtered, and the filtrate was concentrated to give a residue. The residue was purified by prep-HPLC (Phenomenex luna C18 150×25 mm×10 μm; mobile phase: [water (FA)-ACN]; B %: 39%-69%), then the reaction mixture was concentrated to remove MeCN and extracted with EtOAc (50.0 mL×2). The combined organic layers were dried over anhydrous Na₂SO₄, filtered, and concentrated under reduced pressure to give a residue. The compound was separated by SFC (DAICEL CHIRALPAK AD (250 mm×30 mm, 10 μm); 25% mobile phase: [0.1% NH₃H₂O EtOH]) to afford 4 separate peaks. The second eluting, single stereoisomer of N-((1S)-2,2-dicyclopropyl-1-(6-((2-oxo-4-(trifluoromethyl)pyrrolidin-3-yl)methyl)imidazo[1,2-b]pyridazin-2-yl)ethyl)-1-ethyl-1H-pyrazole-5-carboxamide (Compound 44, 61.11 mg, 114 μmol, 33% yield) was obtained as white solid. LCMS [M+H]⁺=530.2 m/z. The third peak of N-((1S)-2,2-dicyclopropyl-1-(6-((2-oxo-4-(trifluoromethyl)pyrrolidin-3-yl)methyl)imidazo[1,2-b]pyridazin-2-yl)ethyl)-1-ethyl-1H-pyrazole-5-carboxamide was collected (Compound 45, 2.50 mg, 4.47 μmol, 1.3% yield) and afforded a white solid, isolated as a mixture of stereoisomers. LCMS [M+H]⁺=530.2 m/z. The fourth eluting, single stereoisomer of N-((1S)-2,2-dicyclopropyl-1-(6-((2-oxo-4-(trifluoromethyl)pyrrolidin-3-yl)methyl)imidazo[1,2-b]pyridazin-2-yl)ethyl)-1-ethyl-1H-pyrazole-5-carboxamide (Compound 46, 3.51 mg, 5.84 μmol, 1.7% yield) was obtained as a white solid. LCMS [M+H]⁺=530.2 m/z.

### Example 55: Preparation of Compounds 47 and 48

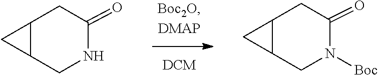 [SMILES: O=C1CC2CC2CN1 ; CC(C)(C)OC(=O)N1CC2CC2CC1=O]

To a solution of 3-azabicyclo[4.1.0]heptan-4-one (800 mg, 7.20 mmol, 1.00 eq) in DCM (8.00 mL) was added Boc₂O (2.36 g, 10.8 mmol, 2.48 mL, 1.50 eq), DMAP (87.9 mg, 719 μmol, 0.10 eq) and TEA (1.09 g, 10.8 mmol, 1.50 mL, 1.50 eq). The reaction mixture was stirred at RT for 12 h. The reaction mixture was partitioned between DCM (80.0 mL) and H₂O (80.0 mL). The organic phase was separated, dried over Na₂SO₄, filtered, and concentrated under reduced pressure to give a residue. The residue was purified by column chromatography (SiO₂, PE/EtOAc=10/1 to 5/1). The product, tert-butyl 4-oxo-3-azabicyclo[4.1.0]heptane-3-carboxylate (1.08 g, 5.11 mmol, 71% yield), was obtained as a yellow oil. ¹H NMR (400 MHz, CDCl₃) δ 3.39-4.01 (m, 1H), 3.84-3.92 (m, 1H), 2.74-2.85 (m, 1H), 2.55-2.67 (m, 1H), 1.52 (s, 9H), 1.20-1.34 (m, 2H), 0.65-0.75 (m, 1H), 0.44-0.51 (m, 1H).

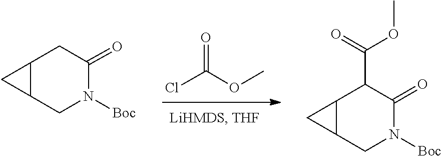

To a solution of tert-butyl 4-oxo-3-azabicyclo[4.1.0]heptane-3-carboxylate (900 mg, 4.26 mmol, 1.00 eq) in THF (10.0 mL) was added LiHMDS (1 M, 8.95 mL, 2.10 eq) at −70° C. for 1 h under N₂ atmosphere, then methyl chloroformate (684 mg, 7.24 mmol, 560 μL, 1.70 eq) was added at −70° C. under N₂ atmosphere. The reaction mixture was stirred at −70° C. for 1 h. The reaction mixture was treated with saturated aqueous NH₄Cl (20.0 mL) at 0° C., and then diluted with H₂O (60.0 mL) and extracted with EtOAc (60.0 mL). The organic phase was separated, dried over Na₂SO₄, filtered, and concentrated under reduced pressure to give a residue. The residue was purified by column chromatography (SiO₂, PE/EtOAc=10/1 to 3/1). The product, 3-(tert-butyl) 5-methyl 4-oxo-3-azabicyclo[4.1.0]heptane-3,5-dicarboxylate (600 mg, 2.23 mmol, 52% yield), was obtained as a yellow oil and used directly in the next step without further purification.

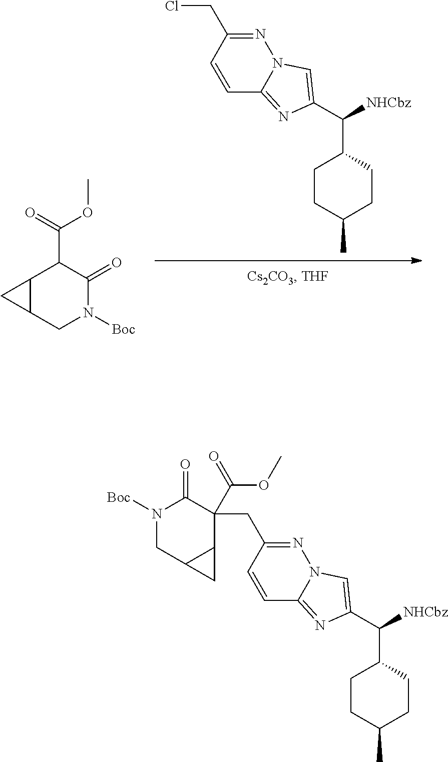

To a solution of 3-(tert-butyl) 5-methyl 4-oxo-3-azabicyclo[4.1.0]heptane-3,5-dicarboxylate (500 mg, 1.86 mmol, 1.00 eq) in THF (15.0 mL) was added Cs₂CO₃ (1.81 g, 5.57 mmol, 3.00 eq) and benzyl ((S)-(6-(chloromethyl)imidazo[1,2-b]pyridazin-2-yl)((1r,4S)-4-methylcyclohexyl)methyl)carbamate (1.19 g, 2.79 mmol, 1.50 eq). The reaction mixture was stirred at 70° C. for 2 h. The reaction mixture was partitioned between EtOAc (100 mL) and H₂O (100 mL). The organic phase was separated, dried over Na₂SO₄, filtered, and concentrated under reduced pressure to give a residue. The residue was purified by column chromatography (SiO₂, PE/EtOAc=10/1 to 1/1). The product, 3-(tert-butyl) 5-methyl 5-((2-((S)-(((benzyloxy)carbonyl)amino)((1r,4S)-4-methylcyclohexyl)methyl)imidazo[1,2-b]pyridazin-6-yl)methyl)-4-oxo-3-azabicyclo[4.1.0]heptane-3,5-dicarboxylate (400 mg, 606 μmol, 33% yield), was obtained as a yellow solid. LCMS [M+H]⁺=660.5 m/z. ¹H NMR (400 MHz, CDCl₃) δ 7.69-7.88 (m, 2H), 7.33-7.41 (m, 4H), 7.28-7.32 (m, 2H), 7.00-7.18 (m, 1H), 5.49-5.70 (m, 1H), 5.01-5.16 (m, 2H), 4.69-4.81 (m, 1H), 4.09-4.33 (m, 1H), 3.75-3.88 (m, 3H), 3.60-3.71 (m, 1H), 3.51-3.58 (m, 1H), 1.81-1.93 (m, 2H), 1.66-1.75 (m, 2H), 1.55-1.62 (m, 9H) 1.43-1.48 (m, 4H), 1.02-1.38 (m, 5H), 0.84-0.87 (m, 4H).

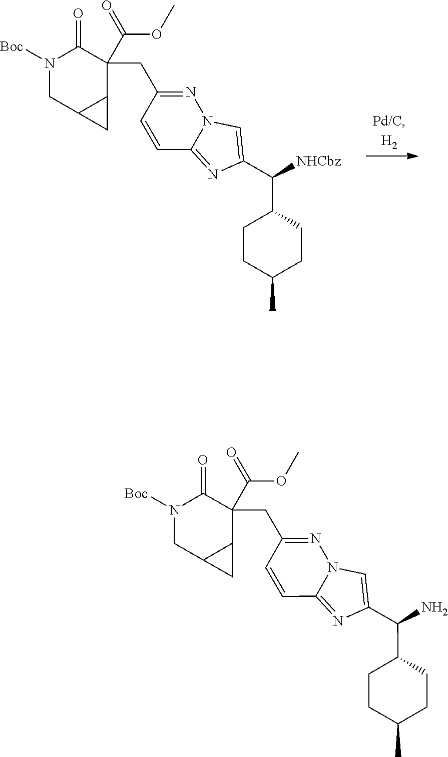 [SMILES: COC(=O)C1(Cc2ccc3nc([C@@H](NC(=O)OCc4ccccc4)[C@H]4CC[C@H](C)CC4)cn3n2)C(=O)N(C(=O)OC(C)(C)C)CC2CC21 ; COC(=O)C1(Cc2ccc3nc([C@@H](N)[C@H]4CC[C@H](C)CC4)cn3n2)C(=O)N(C(=O)OC(C)(C)C)CC2CC21]

To a solution of 3-(tert-butyl) 5-methyl 5-((2-((S)-(((benzyloxy)carbonyl)amino)((1r,4S)-4-methylcyclohexyl)methyl)imidazo[1,2-b]pyridazin-6-yl)methyl)-4-oxo-3-azabicyclo[4.1.0]heptane-3,5-dicarboxylate (400 mg, 606.27 μmol, 1.00 eq) in DCM (10.0 mL) and MeOH (1.00 mL) was added Pd/C (100 mg, 10.0% purity) under N₂ atmosphere. The suspension was degassed and purged with H₂ 3 times. The reaction mixture was stirred under H₂ (15 psi) at RT for 24 h. The reaction was filtered and concentrated under reduced pressure to give a residue. The product, 3-(tert-butyl) 5-methyl 5-((2-((S)-amino((1r,4S)-4-methylcyclohexyl)methyl)imidazo[1,2-b]pyridazin-6-yl)methyl)-4-oxo-3-azabicyclo[4.1.0]heptane-3,5-dicarboxylate (280 mg, 532 μmol, 88% yield), was obtained as a yellow solid. LCMS [M+H]⁺=526.3 m/z.

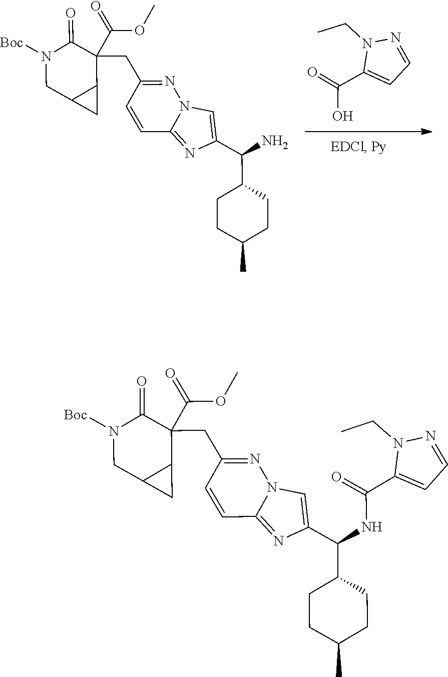

To a solution of 3-(tert-butyl) 5-methyl 5-((2-((S)-amino((1r,4S)-4-methylcyclohexyl)methyl)imidazo[1,2-b]pyridazin-6-yl)methyl)-4-oxo-3-azabicyclo[4.1.0]heptane-3,5-dicarboxylate (230 mg, 437 μmol, 1.00 eq) and 1-ethyl-1H-pyrazole-5-carboxylic acid (91.9 mg, 656 μmol, 1.50 eq) in pyridine (4.00 mL) was added EDCI (251 mg, 1.31 mmol, 3.00 eq). The reaction mixture was stirred at RT for 2 h. The reaction mixture was partitioned between EtOAc (80.0 mL) and H₂O (80.0 mL). The organic phase was separated, dried over Na₂SO₄, filtered, and concentrated under reduced pressure to give a residue. The residue was purified by prep-TLC (SiO₂, DCM/MeOH=10/1). The product, 3-(tert-butyl) 5-methyl 5-((2-((S)-(1-ethyl-1H-pyrazole-5-carboxamido)((1r,4S)-4-methylcyclohexyl)methyl)imidazo[1,2-b]pyridazin-6-yl)methyl)-4-oxo-3-azabicyclo[4.1.0]heptane-3,5-dicarboxylate (140 mg, 216 mol, 49% yield), was obtained as a white solid. LCMS [M+H]⁺=648.2 m/z.

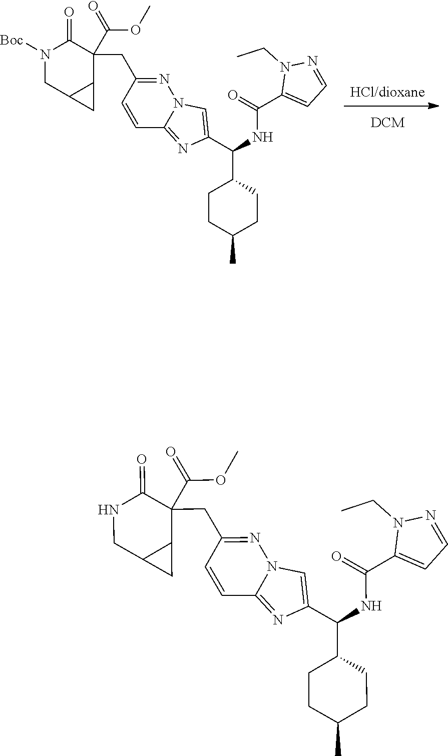 [SMILES: CCn1nccc1C(=O)N[C@H](c1cn2nc(CC3(C(=O)OC)C(=O)N(C(=O)OC(C)(C)C)CC4CC43)ccc2n1)[C@H]1CC[C@H](C)CC1 ; CCn1nccc1C(=O)N[C@H](c1cn2nc(CC3(C(=O)OC)C(=O)NCC4CC43)ccc2n1)[C@H]1CC[C@H](C)CC1]

To a solution of 3-(tert-butyl) 5-methyl 5-((2-((S)-(1-ethyl-1H-pyrazole-5-carboxamido)((1r,4S)-4-methylcyclohexyl)methyl)imidazo[1,2-b]pyridazin-6-yl)methyl)-4-oxo-3-azabicyclo[4.1.0]heptane-3,5-dicarboxylate (140 mg, 216 μmol, 1.00 eq) in DCM (2.00 mL) was added HCl/dioxane (4 M, 2.00 mL). The reaction mixture was stirred at RT for 1 h. The reaction was concentrated under reduced pressure to give a residue. The product, methyl 5-((2-((S)-(1-ethyl-1H-pyrazole-5-carboxamido)((1r,4S)-4-methylcyclohexyl)methyl)imidazo[1,2-b]pyridazin-6-yl)methyl)-4-oxo-3-azabicyclo[4.1.0]heptane-5-carboxylate (120 mg, 205.44 mol, 95% yield, HCl salt), was obtained as a white solid. LCMS [M+H]⁺=548.4 m/z.

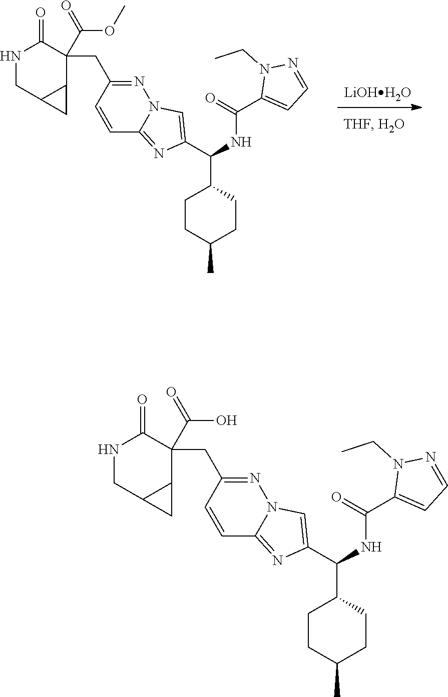

To a solution of methyl 5-((2-((S)-(1-ethyl-1H-pyrazole-5-carboxamido)((1r,4S)-4-methylcyclohexyl)methyl)imidazo[1,2-b]pyridazin-6-yl)methyl)-4-oxo-3-azabicyclo[4.1.0]heptane-5-carboxylate (120 mg, 205 μmol, 1.00 eq, HCl) in THF (1.00 mL) was added LiOH·H₂O (17.2 mg, 410 μmol, 2.00 eq) and H₂O (0.50 mL) at 0° C. The reaction mixture was stirred at RT for 3 h. The reaction was adjusted pH to 3 with 1 M HCl. The reaction mixture was partitioned between EtOAc (60.0 mL) and H₂O (60.0 mL). The organic phase was separated, dried over Na₂SO₄, filtered, and concentrated under reduced pressure to give a residue. The product, 5-((2-((S)-(1-ethyl-1H-pyrazole-5-carboxamido)((1r,4S)-4-methylcyclohexyl)methyl)imidazo[1,2-b]pyridazin-6-yl)methyl)-4-oxo-3-azabicyclo[4.1.0]heptane-5-carboxylic acid (100 mg, 187 μmol, 91% yield), was obtained as a white solid. LCMS [M+H]⁺=534.4 m/z.

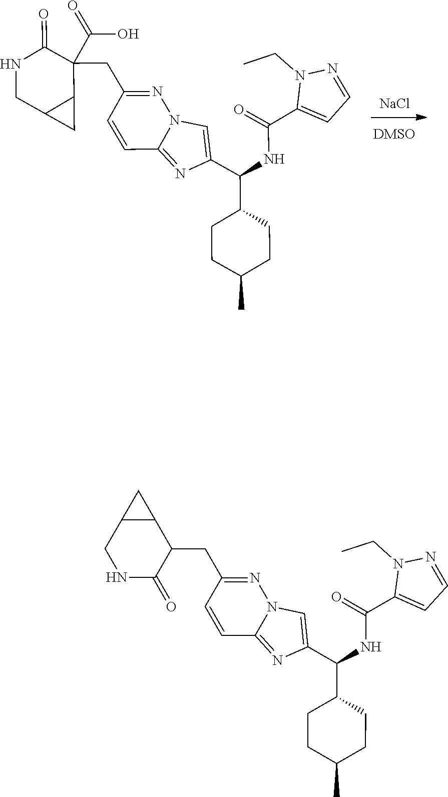

To a solution of 5-((2-((S)-(1-ethyl-1H-pyrazole-5-carboxamido)((1r,4S)-4-methylcyclohexyl)methyl)imidazo[1,2-b]pyridazin-6-yl)methyl)-4-oxo-3-azabicyclo[4.1.0]heptane-5-carboxylic acid (100 mg, 187 μmol, 1.00 eq) in DMSO (1.50 mL) was added NaCl (21.9 mg, 374 μmol, 2.00 eq). The reaction mixture was stirred at 120° C. for 1 h. The reaction mixture was filtered and concentrated under reduced pressure to give a residue. The residue was purified by prep-HPLC (neutral condition; Waters Xbridge 150×25 mm×5 μm; mobile phase: [water (NH₄HCO₃)-ACN]; B %: 39%-69%). The product, 1-ethyl-N-((1S)-((1r,4S)-4-methylcyclohexyl)(6-((4-oxo-3-azabicyclo[4.1.0]heptan-5-yl)methyl)imidazo[1,2-b]pyridazin-2-yl)methyl)-1H-pyrazole-5-carboxamide (56.0 mg, 114 μmol, 61% yield), was obtained as a white solid, isolated as a mixture of stereoisomers. LCMS [M+H]⁺=490.4 m/z.

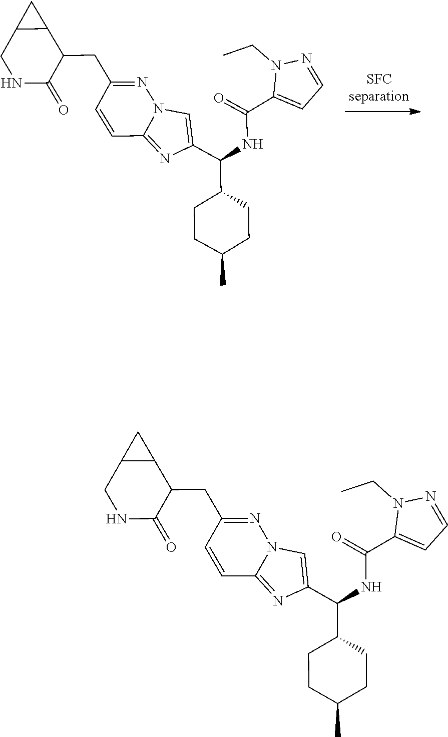

The residue was purified by SFC (condition: DAICEL CHIRALCEL OX (250 mm×30 mm, 10 μm); 45% mobile phase: [0.1% NH₃H₂O EtOH]). The first eluting, single stereoisomers of 1-ethyl-N-((1S)-((1r,4S)-4-methylcyclohexyl)(6-((4-oxo-3-azabicyclo[4.1.0]heptan-5-yl)methyl)imidazo[1,2-b]pyridazin-2-yl)methyl)-1H-pyrazole-5-carboxamide (Compound 47, 20.33 mg, 41.1 μmol, 36% yield) was obtained as a white solid. LCMS [M+H]⁺=490.2 m/z. The second eluting, single stereoisomer of 1-ethyl-N-((1S)-((1r,4S)-4-methylcyclohexyl)(6-((4-oxo-3-azabicyclo[4.1.0]heptan-5-yl)methyl)imidazo[1,2-b]pyridazin-2-yl)methyl)-1H-pyrazole-5-carboxamide (Compound 48, 27.73 mg, 56.0 μmol, 49% yield), was obtained as a white solid. LCMS [M+H]⁺=490.2 m/z.

### Example 56: Preparation of Compounds 49, 50, and 51

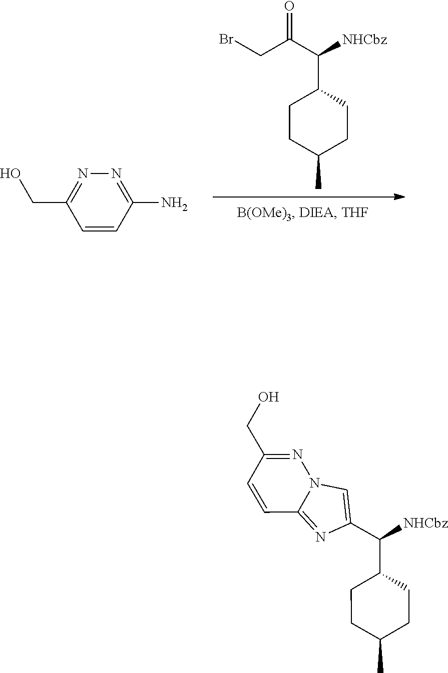 [SMILES: Nc1ccc(CO)nn1 ; C[C@H]1CC[C@H]([C@H](NC(=O)OCc2ccccc2)C(=O)CBr)CC1 ; C[C@H]1CC[C@H]([C@H](NC(=O)OCc2ccccc2)c2cn3nc(CO)ccc3n2)CC1]

To a solution of (6-aminopyridazin-3-yl) MeOH (1.00 g, 4.18 mmol, 1.00 eq, TFA salt) Intermediate 4 (1.92 g, 5.02 mmol, 1.20 eq) in THF (5.00 mL) was added B(OMe)₃ (2.17 g, 20.9 mmol, 2.36 mL, 5.00 eq) and DIEA (2.70 g, 20.9 mmol, 3.64 mL, 5.00 eq). The reaction mixture was stirred at 70° C. for 2 h. The reaction mixture was diluted with H₂O (30.0 mL) and extracted with EtOAc (30.0 mL×3). The combined organic layers were washed with saturated aqueous NaHCO₃ (30.0 mL×2), dried over Na₂SO₄, filtered, and concentrated under reduced pressure to give a residue. The residue was purified by column chromatography (SiO₂, PE/EtOAc=100/1 to 10/1). Benzyl ((S)-(6-(hydroxymethyl)imidazo[1,2-b]pyridazin-2-yl)((1r,4S)-4-methylcyclohexyl)methyl)carbamate (1.50 g, 3.67 mmol, 88% yield) was obtained as a white solid. LCMS [M+H]⁺=409.3 m/z.

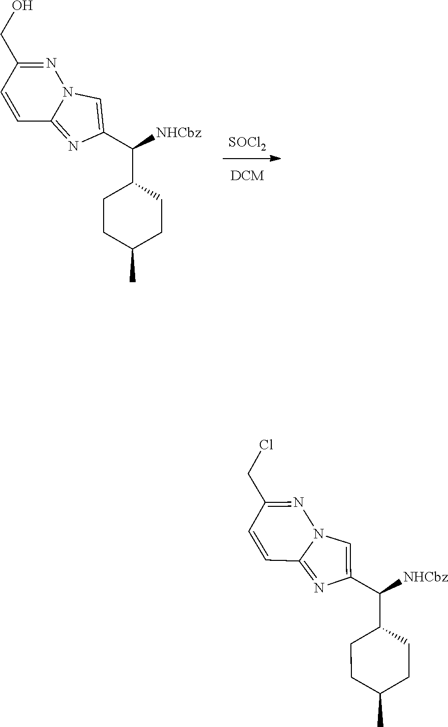

To a solution of benzyl ((S)-(6-(hydroxymethyl)imidazo[1,2-b]pyridazin-2-yl)((1r,4S)-4-methylcyclohexyl)methyl)carbamate (0.400 g, 979 μmol, 1.00 eq) in DCM (2.00 mL) was added SOCl₂ (328 mg, 2.76 mmol, 0.20 mL, 2.82 eq) at 0° C. The reaction mixture was stirred at RT for 1 h. The reaction mixture was concentrated under reduced pressure to give a residue. Benzyl ((S)-(6-(chloromethyl)imidazo[1,2-b]pyridazin-2-yl)((1r,4S)-4-methylcyclohexyl)methyl)carbamate (410 mg, 960 μmol, 98% yield) was obtained as a white solid, which was used directly in the next step without further purification.

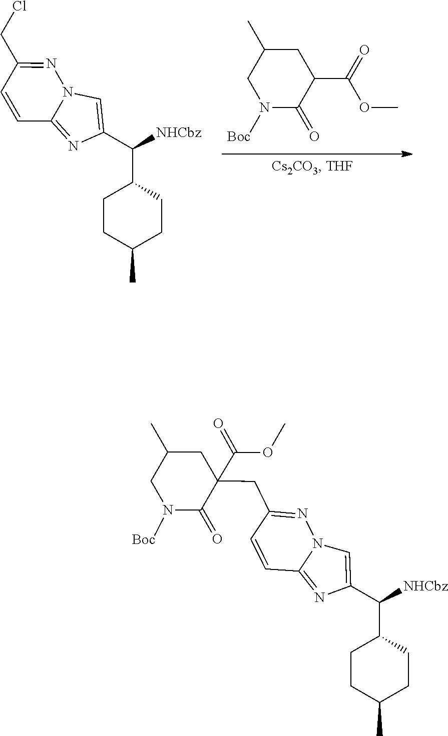 [SMILES: C[C@H]1CC[C@H]([C@H](NC(=O)OCc2ccccc2)c2cn3nc(CCl)ccc3n2)CC1 ; COC(=O)C1CC(C)CN(C(=O)OC(C)(C)C)C1=O ; COC(=O)C1(Cc2ccc3nc([C@@H](NC(=O)OCc4ccccc4)[C@H]4CC[C@H](C)CC4)cn3n2)CC(C)CN(C(=O)OC(C)(C)C)C1=O]

To a solution of 1-(tert-butyl) 3-methyl 5-methyl-2-oxopiperidine-1,3-dicarboxylate (460 mg, 1.08 mmol, 1.00 eq) in THF (5.00 mL) was added Cs₂CO₃ (1.05 g, 3.23 mmol, 3.00 eq). The reaction mixture was stirred at RT for 30 mins, then benzyl ((S)-(6-(chloromethyl)imidazo[1,2-b]pyridazin-2-yl)((1r,4S)-4-methylcyclohexyl)methyl)carbamate (350 mg, 1.29 mmol, 1.20 eq) was added and the reaction mixture was stirred at 70° C. for 2 h. The reaction mixture was diluted with H₂O (30.0 mL) and extracted with EtOAc (30.0 mL×3). The combined organic layers were washed with saturated aqueous NaHCO₃ (30.0 mL×2), dried over Na₂SO₄, filtered, and concentrated under reduced pressure to give a residue. The residue was purified by column chromatography (SiO₂, PE/EtOAc=100/1 to 10/1). The product, 1-(tert-butyl) 3-methyl 3-((2-((S)-(((benzyloxy)carbonyl)amino)((1r,4S)-4-methylcyclohexyl)methyl)imidazo[1,2-b]pyridazin-6-yl)methyl)-5-methyl-2-oxopiperidine-1,3-dicarboxylate (500 mg, 755 μmol, 70% yield), was obtained as a white solid. LCMS [M+H]⁺=662.5 m/z.

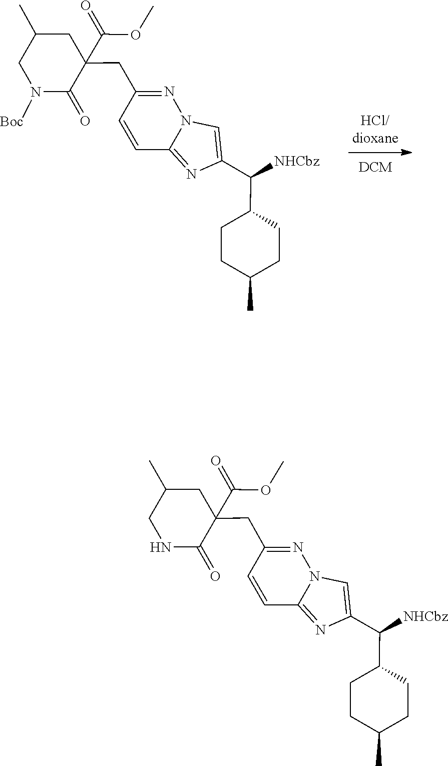 [SMILES: COC(=O)C1(Cc2ccc3nc([C@@H](NC(=O)OCc4ccccc4)[C@H]4CC[C@H](C)CC4)cn3n2)CC(C)CN(C(=O)OC(C)(C)C)C1=O ; COC(=O)C1(Cc2ccc3nc([C@@H](NC(=O)OCc4ccccc4)[C@H]4CC[C@H](C)CC4)cn3n2)CC(C)CNC1=O]

To a solution of 1-(tert-butyl) 3-methyl 3-((2-((S)-(((benzyloxy)carbonyl)amino)((1r,4S)-4-methylcyclohexyl)methyl)imidazo[1,2-b]pyridazin-6-yl)methyl)-5-methyl-2-oxopiperidine-1,3-dicarboxylate (500 mg, 755 μmol, 1.00 eq) in DCM (5.00 mL) was added HCl/dioxane (4 M, 4.44 mL, 23.5 eq) at 0° C. The reaction mixture was stirred at RT for 1 h. The reaction mixture was concentrated under reduced pressure to give a residue. Methyl 3-((2-((S)-(((benzyloxy)carbonyl)amino)((1r,4S)-4-methylcyclohexyl)methyl)imidazo[1,2-b]pyridazin-6-yl)methyl)-5-methyl-2-oxopiperidine-3-carboxylate (450 mg, 752 μmol, quantitative, HCl salt) was obtained as a white solid. LCMS [M+H]⁺=562.4 m/z.

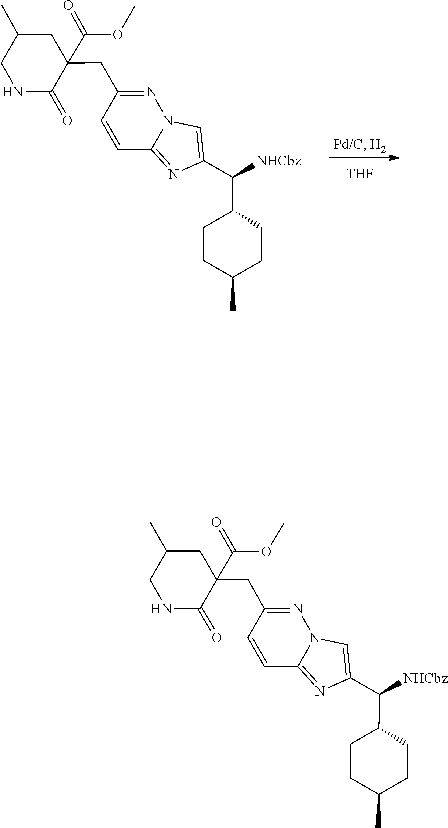 [SMILES: COC(=O)C1(Cc2ccc3nc([C@@H](NC(=O)OCc4ccccc4)[C@H]4CC[C@H](C)CC4)cn3n2)CC(C)CNC1=O ; "THF" ; COC(=O)C1(Cc2ccc3nc([C@@H](NC(=O)OCc4ccccc4)[C@H]4CC[C@H](C)CC4)cn3n2)CC(C)CNC1=O]

To a solution of methyl 3-((2-((S)-(((benzyloxy)carbonyl)amino)((1r,4S)-4-methylcyclohexyl)methyl)imidazo[1,2-b]pyridazin-6-yl)methyl)-5-methyl-2-oxopiperidine-3-carboxylate (450 mg, 752 μmol, 1.00 eq, HCl salt) in THF (5.00 mL) was added Pd/C (45.0 mg 10.0% purity) under N₂ atmosphere. The suspension was degassed and purged with H₂ 3 times. The reaction mixture was stirred under H₂ (15 psi) at RT for 1 h. The reaction mixture was filtered, and the cake was washed with 100 mL MeOH. The filtrate was then concentrated under reduced pressure to give a residue. Methyl 3-((2-((S)-(((benzyloxy)carbonyl)amino)((1r,4S)-4-methylcyclohexyl)methyl)imidazo[1,2-b]pyridazin-6-yl)methyl)-5-methyl-2-oxopiperidine-3-carboxylate (320 mg, 748 μmol, 99% yield) was obtained as a white solid. LCMS [M+H]⁺=428.1 m/z.

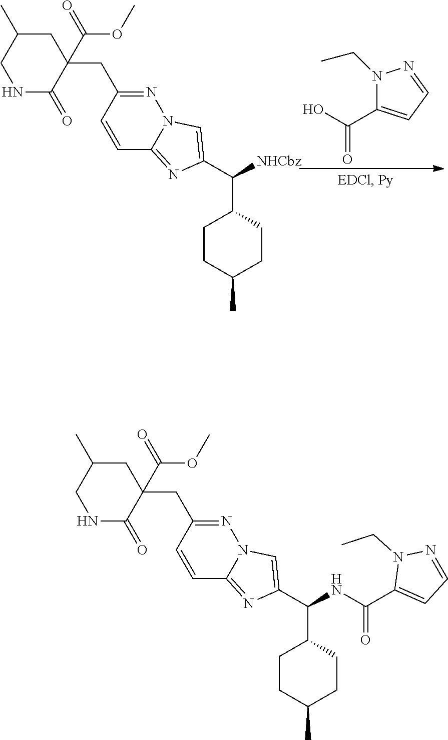

Methyl 3-((2-((S)-(1-ethyl-1H-pyrazole-5-carboxamido)((1r,4S)-4-methylcyclohexyl)methyl)imidazo[1,2-b]pyridazin-6-yl)methyl)-5-methyl-2-oxopiperidine-3-carboxylate was synthesized following General Procedure 4, using methyl 3-((2-((S)-(((benzyloxy)carbonyl)amino)((1r,4S)-4-methylcyclohexyl)methyl)imidazo[1,2-b]pyridazin-6-yl)methyl)-5-methyl-2-oxopiperidine-3-carboxylate (320 mg, 748 μmol, 1.00 eq), 1-ethyl-1H-pyrazole-5-carboxylic acid (157 mg, 1.12 mmol, 1.50 eq), EDCI (430 mg, 2.25 mmol, 3.00 eq), and pyridine (5.00 mL). The reaction mixture was stirred for 1 h. The residue was purified by prep-TLC (SiO₂, PE/EtOAc=1/1). The product, methyl 3-((2-((S)-(1-ethyl-1H-pyrazole-5-carboxamido)((1r,4S)-4-methylcyclohexyl)methyl)imidazo[1,2-b]pyridazin-6-yl)methyl)-5-methyl-2-oxopiperidine-3-carboxylate (350 mg, 636 μmol, 86% yield), was obtained as a white solid. LCMS [M+H]⁺=550.4 m/z.

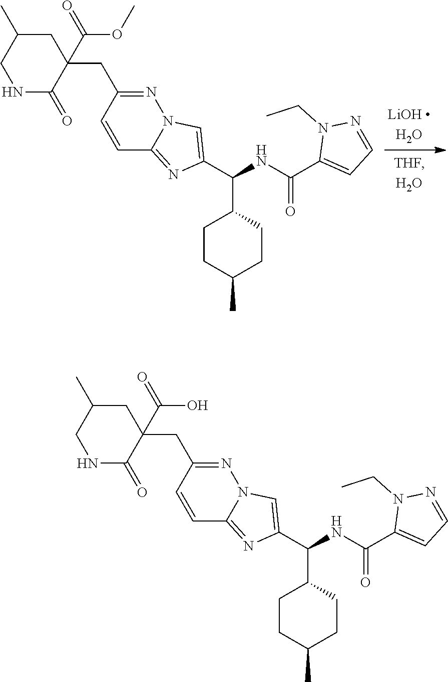 [SMILES: CCn1nccc1C(=O)N[C@H](c1cn2nc(CC3(C(=O)OC)CC(C)CNC3=O)ccc2n1)[C@H]1CC[C@H](C)CC1 ; CCn1nccc1C(=O)N[C@H](c1cn2nc(CC3(C(=O)O)CC(C)CNC3=O)ccc2n1)[C@H]1CC[C@H](C)CC1]

To a solution of methyl 3-((2-((S)-(1-ethyl-1H-pyrazole-5-carboxamido)((1r,4S)-4-methylcyclohexyl)methyl)imidazo[1,2-b]pyridazin-6-yl)methyl)-5-methyl-2-oxopiperidine-3-carboxylate (350 mg, 636 μmol, 1.00 eq) in THF (3.00 mL) was added LiOH·H₂O (106 mg, 2.55 mmol, 4.00 eq) in H₂O (1.00 mL) at 0° C. The reaction mixture was stirred at RT for 1 h. The reaction mixture was diluted with H₂O (10.0 mL) and 1 M HCl to adjusted pH to 2. The reaction mixture was extracted with DCM (20.0 mL×2). The combined organic layers were dried over Na₂SO₄, filtered, and concentrated under reduced pressure to give a residue. The product, 3-((2-((S)-(1-ethyl-1H-pyrazole-5-carboxamido)((1r,4S)-4-methylcyclohexyl)methyl)imidazo[1,2-b]pyridazin-6-yl)methyl)-5-methyl-2-oxopiperidine-3-carboxylic acid (300 mg, 560 μmol, 88% yield) was obtained as a white solid. LCMS [M+H]⁺=536.3 m/z.

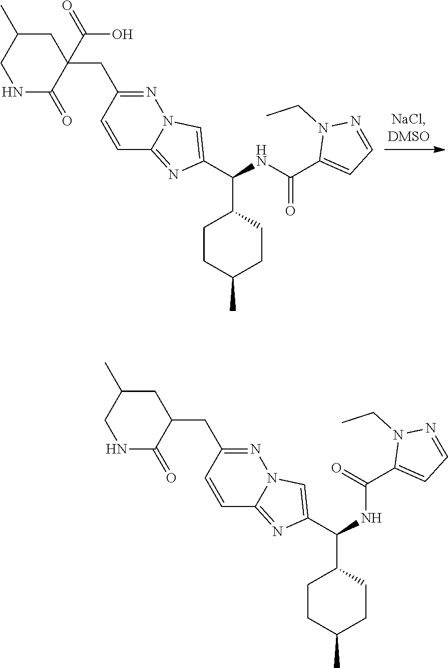 [SMILES: CCn1nccc1C(=O)N[C@H](c1cn2nc(CC3(C(=O)O)CC(C)CNC3=O)ccc2n1)[C@H]1CC[C@H](C)CC1 ; CCn1nccc1C(=O)N[C@H](c1cn2nc(CC3CC(C)CNC3=O)ccc2n1)[C@H]1CC[C@H](C)CC1]

To a solution of 3-((2-((S)-(1-ethyl-1H-pyrazole-5-carboxamido)((1r,4S)-4-methylcyclohexyl)methyl)imidazo[1,2-b]pyridazin-6-yl)methyl)-5-methyl-2-oxopiperidine-3-carboxylic acid (300 mg, 560 μmol, 1.00 eq) in DMSO (5.00 mL) was added NaCl (163 mg, 2.80 mmol, 5.00 eq). The reaction mixture was stirred at 100° C. for 1 h. The reaction mixture was diluted with H₂O (30.0 mL) and extracted with EtOAc (30.0 mL×3). The combined organic layers were washed with brine (30.0 mL×2), dried over Na₂SO₄, filtered, and concentrated under reduced pressure to give a residue. The residue was purified by prep-TLC (SiO₂, DCM/MeOH=10/1) then (DAICEL CHIRALPAK IC (250 mm×30 mm, 10 μm); 40% mobile phase: [ACN/EtOH (0.1% NH₃H₂O)]). A second SFC purification was carried out (DAICEL CHIRALPAK AD (250 mm×30 mm, 10 μm); 35% mobile phase: [0.1% NH₃H₂O EtOH]). The mixture of stereoisomers, 1-ethyl-N-((1S)-(6-((5-methyl-2-oxopiperidin-3-yl)methyl)imidazo[1,2-b]pyridazin-2-yl)((1r,4S)-4-methylcyclohexyl)methyl)-1H-pyrazole-5-carboxamide (152 mg, 56.46 μmol, 10% yield), was obtained as a white solid. LCMS [M+H]⁺=492.3 m/z. This mixture of stereoisomers was separated by SFC (DAICEL CHIRALPAK IC (250 mm×30 mm, 10 μm); 40% mobile phase: [ACN/EtOH (0.1% NH₃H₂O)]). A second SFC purification was carried out (DAICEL CHIRALPAK AD (250 mm×30 mm, 10 μm); 35% mobile phase: [0.1% NH₃H₂O EtOH]). The second eluting, single stereoisomer of 1-ethyl-N-((1S)-(6-((5-methyl-2-oxopiperidin-3-yl)methyl)imidazo[1,2-b]pyridazin-2-yl)((1r,4S)-4-methylcyclohexyl)methyl)-1H-pyrazole-5-carboxamide (Compound 49, 21.7 mg, 43.1 μmol, 7.7% yield) was obtained as a white solid. LCMS [M+H]⁺=492.3 m/z. The third eluting, single stereoisomer of 1-ethyl-N-((1S)-(6-((5-methyl-2-oxopiperidin-3-yl)methyl)imidazo[1,2-b]pyridazin-2-yl)((1r,4S)-4-methylcyclohexyl)methyl)-1H-pyrazole-5-carboxamide (Compound 50, 27.6 mg, 54. μmol, 9.7% yield) was obtained as a white solid. LCMS [M+H]⁺=492.3 m/z. The fourth eluting, single stereoisomer of 1-ethyl-N-((1S)-(6-((5-methyl-2-oxopiperidin-3-yl)methyl)imidazo[1,2-b]pyridazin-2-yl)((1r,4S)-4-methylcyclohexyl)methyl)-1H-pyrazole-5-carboxamide (Compound 51, 32.7 mg, 65.4 μmol, 12% yield) was obtained as a white solid. LCMS [M+H]⁺=492.4 m/z.

### Example 57: Preparation of Compounds 52, 53, and 54

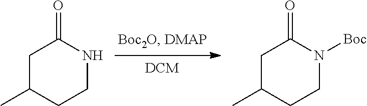

To a solution of 4-methylpiperidin-2-one (2.50 g, 22.1 mmol, 1.00 eq) in DCM (30.0 mL) was added Boc₂O (5.79 g, 26.5 mmol, 6.09 mL, 1.20 eq) and DMAP (540 mg, 4.42 mmol, 0.200 eq), the reaction mixture was stirred at RT for 3 h. The reaction mixture was filtered and concentrated under reduced pressure to give a residue. The residue was purified by column chromatography (SiO2, PE/EtOAc=100/1 to 3/1). The product, tert-butyl 4-methyl-2-oxopiperidine-1-carboxylate (2.50 g, 11.7 mmol, 53% yield), was obtained as a light yellow solid. ¹H NMR (400 MHz, CDCl₃) δ 3.83-3.78 (m, 1H), 3.56-3.49 (m, 1H), 2.58-2.52 (m, 1H), 2.15-2.08 (m, 1H), 2.04-1.91 (m, 2H), 1.51 (s, 9H), 1.49-1.41 (m, 1H), 1.02 (d, J=6.4 Hz, 3H).

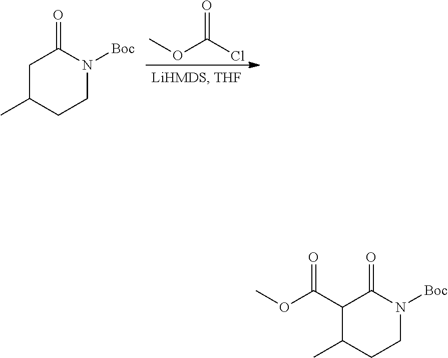

To a solution of tert-butyl 4-methyl-2-oxopiperidine-1-carboxylate (2.50 g, 11.7 mmol, 1.00 eq) in THF (40.0 mL) was added LiHMDS (1 M, 29.3 mL, 2.50 eq) at −78° C. After stirring for 30 min there was added methyl chloroformate (3.05 g, 32.3 mmol, 2.50 mL, 2.75 eq). The reaction mixture was further stirred at −78° C. for 2 h. The reaction mixture was quenched by saturated aqueous NH₄Cl (300 mL) and extracted with EtOAc (300 mL×3), dried over Na₂SO₄, filtered, and concentrated under reduced pressure to give a residue. The residue was purified by column chromatography (SiO₂, PE/EtOAc=100/1 to 3/1). The product, 1-(tert-butyl) 3-methyl 4-methyl-2-oxopiperidine-1,3-dicarboxylate (2.10 g, 7.74 mmol, 66% yield), was obtained as a light yellow oil. ¹H NMR (400 MHz, CDCl₃) δ 3.83-3.76 (m, 1H), 3.75 (s, 3H), 3.62-3.54 (m, 1H), 3.23 (d, J=11.2 Hz, 1H), 2.32-3.24 (m, 1H), 2.01-1.95 (m, 1H), 1.64-1.54 (m, 1H), 1.51 (s, 9H), 1.03 (d, J=6.8 Hz, 3H).

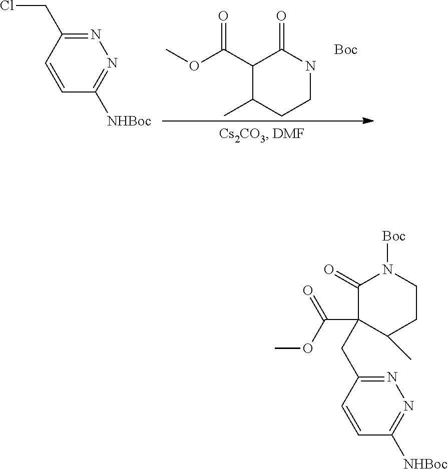 [SMILES: CC(C)(C)OC(=O)Nc1ccc(CCl)nn1 ; COC(=O)C1C(=O)NCCC1C ; "Boc" ; COC(=O)C1(Cc2ccc(NC(=O)OC(C)(C)C)nn2)C(=O)N(C(=O)OC(C)(C)C)CCC1C]

A solution of 1-(tert-butyl) 3-methyl 4-methyl-2-oxopiperidine-1,3-dicarboxylate (1.34 g 4.92 mmol, 1.20 eq) and Cs₂CO₃ (4.01 g, 12.3 mmol, 3.00 eq) in DMF (20.0 mL) was stirred for 30 min at RT. Then tert-butyl (6-(chloromethyl)pyridazin-3-yl)carbamate (1.00 g, 4.10 mmol, 1.00 eq) was added and the reaction mixture was stirred at 60° C. for 1 h. The reaction mixture was diluted with EtOAc (100 mL). The organic layer was washed with H₂O (50.0 mL×3), dried over Na₂SO₄, filtered, and concentrated under reduced pressure to give a residue. The residue was purified by column chromatography (SiO₂, DCM:MeOH=100/1 to 10/1). The product, 1-(tert-butyl) 3-methyl 3-((6-((tert-butoxycarbonyl)amino)pyridazin-3-yl)methyl)-4-methyl-2-oxopiperidine-1,3-dicarboxylate (0.850 g, crude), was obtained as a white solid, which was used directly in the next step without further purification.

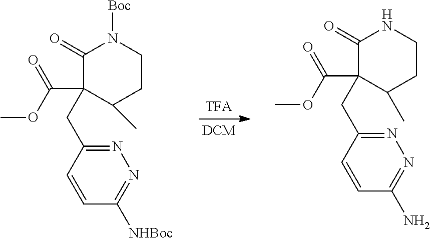 [SMILES: COC(=O)C1(Cc2ccc(NC(=O)OC(C)(C)C)nn2)C(=O)N(C(=O)OC(C)(C)C)CCC1C ; COC(=O)C1(Cc2ccc(N)nn2)C(=O)NCCC1C]

To a solution of 1-(tert-butyl) 3-methyl 3-((6-((tert-butoxycarbonyl)amino)pyridazin-3-yl)methyl)-4-methyl-2-oxopiperidine-1,3-dicarboxylate (0.700 g, crude) in DCM (5.00 mL) was added TFA (7.70 g, 67.5 mmol, 5.00 mL, 46.2 eq) at 0° C. The reaction mixture was stirred at RT for 10 h. The reaction mixture was then concentrated under reduced pressure to give a residue. The product, methyl 3-((6-aminopyridazin-3-yl)methyl)-4-methyl-2-oxopiperidine-3-carboxylate (0.570 g, 1.45 mmol, 50% yield over 2 steps, TFA salt), was obtained as a white solid. LCMS [M+H]⁺=279.2 m/z.

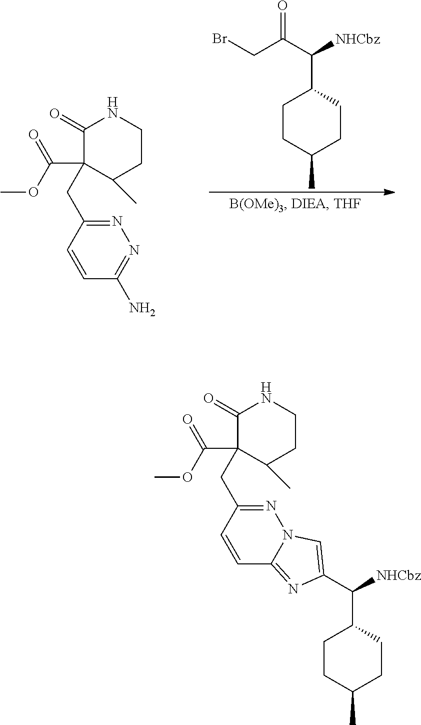 [SMILES: COC(=O)C1(Cc2ccc(N)nn2)C(=O)NCCC1C ; C[C@H]1CC[C@H]([C@H](NC(=O)OCc2ccccc2)C(=O)CBr)CC1 ; COC(=O)C1(Cc2ccc3nc([C@@H](NC(=O)OCc4ccccc4)[C@H]4CC[C@H](C)CC4)cn3n2)C(=O)NCCC1C]

To a solution of methyl 3-((6-aminopyridazin-3-yl)methyl)-4-methyl-2-oxopiperidine-3-carboxylate (0.570 g, 1.45 mmol, 1.00 eq, TFA salt), Intermediate 4 (666 mg, 1.74 mmol, 1.20 eq) in THF (10.0 mL) was added B(OMe)₃ (755 mg, 7.26 mmol, 820 μL, 5.00 eq) and DIEA (939 mg, 7.26 mmol, 1.27 mL, 5.00 eq). The reaction mixture was stirred at 70° C. for 1 h. The reaction mixture was diluted with H₂O (20.0 mL) and extracted with EtOAc (30.0 mL×3). The combined organic layers were dried over Na₂SO₄, filtered, and concentrated under reduced pressure to give a residue. The residue was purified by prep-TLC (DCM:MeOH=10:1). The product, methyl 3-((2-((S)-(((benzyloxy)carbonyl)amino)((1r,4S)-4-methylcyclohexyl)methyl)imidazo[1,2-b]pyridazin-6-yl)methyl)-4-methyl-2-oxopiperidine-3-carboxylate (0.400 g, 712 μmol, 49% yield), was obtained as a yellow solid. LCMS [M+H]⁺=562.4 m/z.

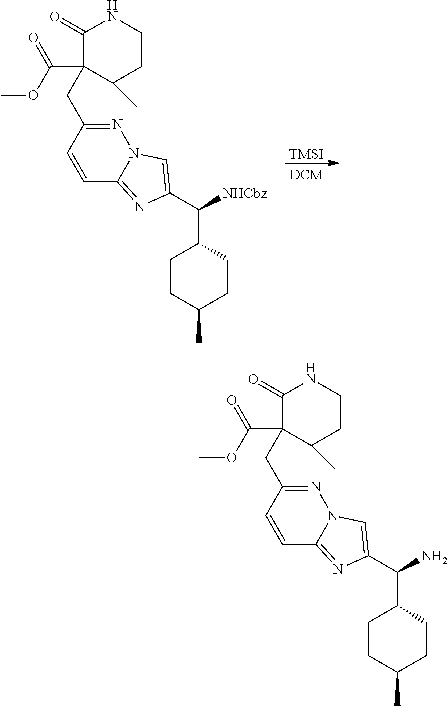 [SMILES: "TMSI" ; COC(=O)C1(Cc2ccc3nc([C@@H](NC(=O)OCc4ccccc4)[C@H]4CC[C@H](C)CC4)cn3n2)C(=O)NCCC1C ; COC(=O)C1(Cc2ccc3nc([C@@H](N)[C@H]4CC[C@H](C)CC4)cn3n2)C(=O)NCCC1C]

Intermediate methyl 3-((2-((S)-amino((1r,4S)-4-methylcyclohexyl)methyl)imidazo[1,2-b]pyridazin-6-yl)methyl)-4-methyl-2-oxopiperidine-3-carboxylate was synthesized following General Procedure 2, using methyl 3-((2-((S)-(((benzyloxy)carbonyl)amino)((1r,4S)-4-methylcyclohexyl)methyl)imidazo[1,2-b]pyridazin-6-yl)methyl)-4-methyl-2-oxopiperidine-3-carboxylate (0.400 g, 712 μmol, 1.00 eq), TMSI (285 mg, 1.42 mmol, 194 μL, 2.00 eq), and DCM (10.0 mL). The product, methyl 3-((2-((S)-amino((1r,4S)-4-methylcyclohexyl)methyl)imidazo[1,2-b]pyridazin-6-yl)methyl)-4-methyl-2-oxopiperidine-3-carboxylate (0.280 g, 655 μmol, 92% yield), was obtained as a yellow solid. LCMS [M+H]⁺=428.4 m/z.

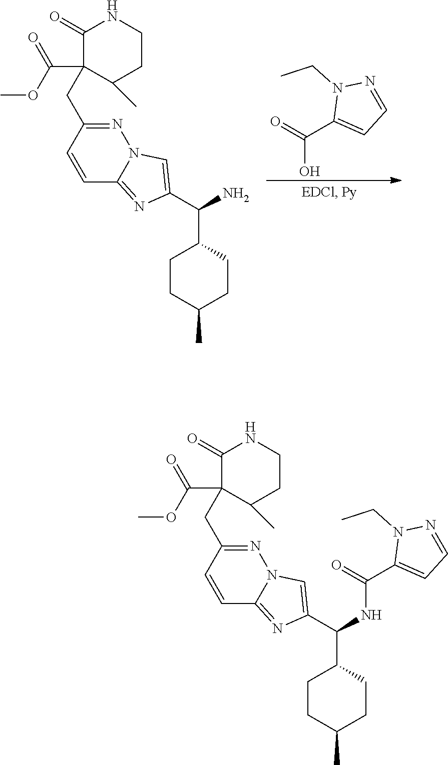

Intermediate methyl 3-((2-((S)-(1-ethyl-1H-pyrazole-5-carboxamido)((1r,4S)-4-methylcyclohexyl)methyl)imidazo[1,2-b]pyridazin-6-yl)methyl)-4-methyl-2-oxopiperidine-3-carboxylate was synthesized following General Procedure 4, using methyl 3-((2-((S)-amino((1r,4S)-4-methylcyclohexyl)methyl)imidazo[1,2-b]pyridazin-6-yl)methyl)-4-methyl-2-oxopiperidine-3-carboxylate (0.280 g, 655 μmol, 1.00 eq), 1-ethyl-1H-pyrazole-5-carboxylic acid (138 mg, 982 μmol, 1.50 eq), EDCI (377 mg, 1.96 mmol, 3.00 eq), and pyridine (8.00 mL). The reaction mixture was stirred for 5 h. The residue was purified by prep-TLC (DCM:MeOH=10:1). The product, methyl 3-((2-((S)-(1-ethyl-1H-pyrazole-5-carboxamido)((1r,4S)-4-methylcyclohexyl)methyl)imidazo[1,2-b]pyridazin-6-yl)methyl)-4-methyl-2-oxopiperidine-3-carboxylate (0.230 g, 413 μmol, 63% yield), was obtained as a yellow solid. LCMS [M+H]⁺=550.8 m/z.

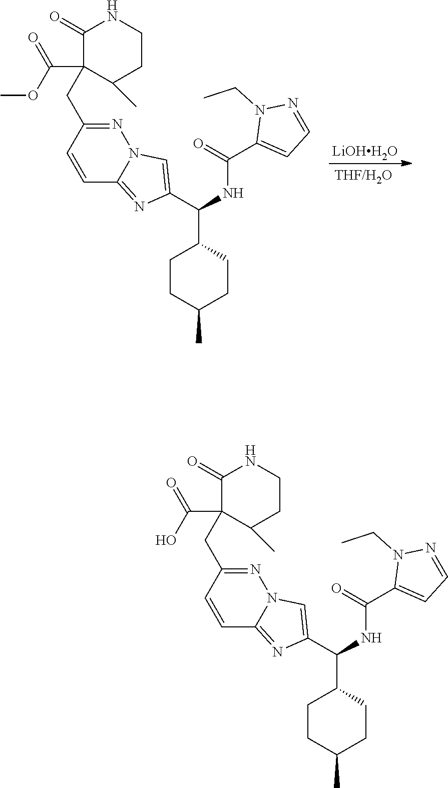

Intermediate 3-((2-((S)-(1-ethyl-1H-pyrazole-5-carboxamido)((1r,4S)-4-methylcyclohexyl)methyl)imidazo[1,2-b]pyridazin-6-yl)methyl)-4-methyl-2-oxopiperidine-3-carboxylic acid was synthesized following General Procedure 5, using methyl 3-((2-((S)-(1-ethyl-1H-pyrazole-5-carboxamido)((1r,4S)-4-methylcyclohexyl)methyl)imidazo[1,2-b]pyridazin-6-yl)methyl)-4-methyl-2-oxopiperidine-3-carboxylate (230 mg, 413 μmol, 1.00 eq) and LiOH·H₂O (52.0 mg, 1.24 mmol, 3.00 eq), stirring for 48 h. The product, 3-((2-((S)-(1-ethyl-1H-pyrazole-5-carboxamido)((1r,4S)-4-methylcyclohexyl)methyl)imidazo[1,2-b]pyridazin-6-yl)methyl)-4-methyl-2-oxopiperidine-3-carboxylic acid (170 mg, 317 μmol, 77% yield), was obtained as a white solid. LCMS [M+H]⁺=536.3 m/z.

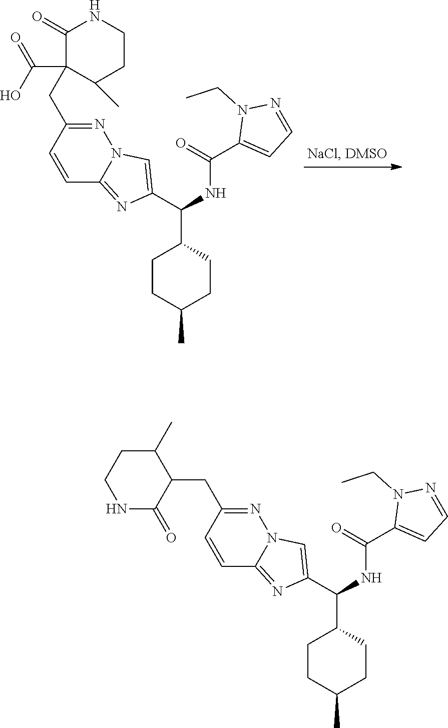 [SMILES: CCn1nccc1C(=O)N[C@H](c1cn2nc(CC3(C(=O)O)C(=O)NCCC3C)ccc2n1)[C@H]1CC[C@H](C)CC1 ; CCn1nccc1C(=O)N[C@H](c1cn2nc(CC3C(=O)NCCC3C)ccc2n1)[C@H]1CC[C@H](C)CC1]

The mixture of stereoisomers, 1-ethyl-N-((1S)-(6-((4-methyl-2-oxopiperidin-3-yl)methyl)imidazo[1,2-b]pyridazin-2-yl)((1r,4S)-4-methylcyclohexyl)methyl)-1H-pyrazole-5-carboxamide (150 mg, 305 μmol, 96% yield), was synthesized following General Procedure 6, using 3-((2-((S)-(1-ethyl-1H-pyrazole-5-carboxamido)((1r,4S)-4-methylcyclohexyl)methyl)imidazo[1,2-b]pyridazin-6-yl)methyl)-4-methyl-2-oxopiperidine-3-carboxylic acid (170 mg, 317 μmol, 1.00 eq), DMSO (2.00 mL), and NaCl (37.1 mg, 635 μmol, 2.00 eq), stirring at 120° C. for 30 min. The mixture of stereoisomers, 1-ethyl-N-((1S)-(6-((4-methyl-2-oxopiperidin-3-yl)methyl)imidazo[1,2-b]pyridazin-2-yl)((1r,4S)-4-methylcyclohexyl)methyl)-1H-pyrazole-5-carboxamide (150 mg, 305 μmol, 96% yield), was obtained as a white solid. LCMS [M+H]⁺=492.4 m/z.

The stereoisomeric mixture was then purified by prep-SFC (DAICEL CHIRALPAK AD (250 mm×30 mm, 10 μm); 45% mobile phase: [0.1% NH₃·H₂O EtOH]), then again purified by prep-SFC (REGIS (S,S) WHELK-01 (250 mm×25 mm, 10 μm); 50% mobile phase: [0.1% NH₃H₂O EtOH]), and once more by prep-SFC (DAICEL CHIRALPAK AS (250 mm×30 mm, 10 μm); 35% mobile phase: [0.10% NH₃H₂O MeOH]). The second eluting stereoisomer of 1-ethyl-N-((1S)-(6-((4-methyl-2-oxopiperidin-3-yl)methyl)imidazo[1,2-b]pyridazin-2-yl)((1r,4S)-4-methylcyclohexyl)methyl)-1H-pyrazole-5-carboxamide (Compound 52, 2.45 mg, 4.98 μmol, 1.6% yield) was obtained as a yellow solid. LCMS [M+H]⁺=492.3 m/z. The third eluting stereoisomer of 1-ethyl-N-((1S)-(6-((4-methyl-2-oxopiperidin-3-yl)methyl)imidazo[1,2-b]pyridazin-2-yl)((1r,4S)-4-methylcyclohexyl)methyl)-1H-pyrazole-5-carboxamide (Compound 53, 26.97 mg, 54.86 μmol, 18% yield) was obtained as a yellow solid. LCMS [M+H]⁺=492.4 m z. The fourth eluting stereoisomer of 1-ethyl-N-((1S)-(6-((4-methyl-2-oxopiperidin-3-yl)methyl)imidazo[1,2-b]pyridazin-2-yl)((1r,4S)-4-methylcyclohexyl)methyl)-1H-pyrazole-5-carboxamide (Compound 54, 5.65 mg, 11.49 μmol, 3.8% yield) was obtained as a white solid. LCMS [M+H]⁺=492.3 m/z.

### Example 58: Preparation of Compounds 55, 56, and 57

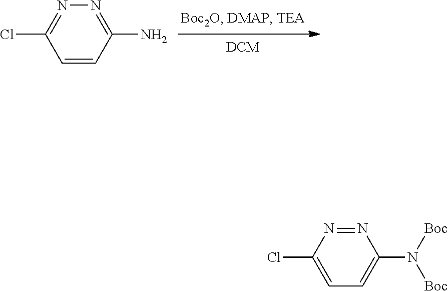 [SMILES: Nc1ccc(Cl)nn1 ; CC(C)(C)OC(=O)N(C(=O)OC(C)(C)C)c1ccc(Cl)nn1]

To a solution of 6-chloropyridazin-3-amine (17.0 g, 131 mmol, 1.00 eq) in DCM (170 mL) was added Boc₂O (57.3 g, 262 mmol, 60.3 mL, 2.00 eq), TEA (26.5 g, 262 mmol, 36.5 mL, 2.00 eq), and DMAP (801 mg, 6.56 mmol, 0.0500 eq). The reaction mixture was stirred at RT for 12 h. The reaction mixture was diluted with H₂O (200 mL) and extracted with DCM (200 mL×3), the combined organic layers were dried over Na₂SO₄ and concentrated to give a residue. The residue was purified by column chromatography (SiO₂, PE:EtOAc=1:0 to 3:1). The product, tert-butyl (tert-butoxycarbonyl)(6-chloropyridazin-3-yl)carbamate (27.0 g, 81.8 mmol, 62% yield), was obtained as a light-yellow solid. ¹H NMR (400 MHz, CDCl₃) δ 7.54 (d, J=1.6 Hz, 2H), 1.47 (s, 18H). LCMS [M+H]⁺=329.9 m/z.

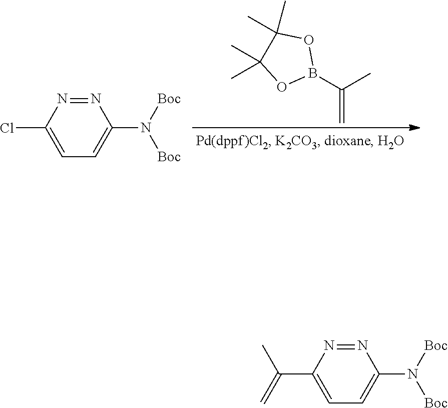 [SMILES: CC(C)(C)OC(=O)N(C(=O)OC(C)(C)C)c1ccc(Cl)nn1 ; C=C(C)B1OC(C)(C)C(C)(C)O1 ; C=C(C)c1ccc(N(C(=O)OC(C)(C)C)C(=O)OC(C)(C)C)nn1]

A mixture of tert-butyl (tert-butoxycarbonyl)(6-chloropyridazin-3-yl)carbamate (3.00 g 9.10 mmol, 1.00 eq), 4,4,5,5-tetramethyl-2-(prop-1-en-2-yl)-1,3,2-dioxaborolane (1.68 g, 10.0 mmol, 1.10 eq), Pd(dppf)Cl₂ (665 mg, 909 μmol, 0.100 eq), and K₂CO₃ (3.77 g, 27.3 mmol, 3.00 eq) in dioxane (30.0 mL) and H₂O (12.0 mL) was degassed and purged with N₂ 3 times, and then the reaction mixture was stirred at 85° C. for 12 h under N₂ atmosphere. The reaction mixture was diluted with water (60.0 mL) and extracted with EtOAc (60.0 mL×2). The combined organic layers were washed with brine (60.0 mL), dried over Na₂SO₄, filtered, and concentrated under reduced pressure to give a residue. The residue was purified by column chromatography (SiO₂, PE:EtOAc=10:1 to 5:1). The product, tert-butyl (tert-butoxycarbonyl)(6-(prop-1-en-2-yl)pyridazin-3-yl)carbamate (2.50 g, 7.45 mmol, 82% yield), was obtained as a white solid. LCMS [M+H]⁺=336.0 m/z.

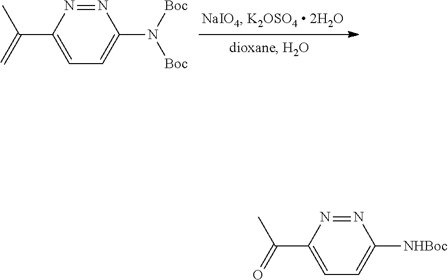 [SMILES: C=C(C)c1ccc(N(C(=O)OC(C)(C)C)C(=O)OC(C)(C)C)nn1 ; CC(=O)c1ccc(NC(=O)OC(C)(C)C)nn1]

To a solution of tert-butyl (tert-butoxycarbonyl)(6-(prop-i-en-2-yl)pyridazin-3-yl)carbamate (1.95 g, 5.81 mmol, 1.00 eq) in dioxane (20.0 mL) and H₂O (7.00 mL) was added NaIO₄ (4.97 g, 23.2 mmol, 1.29 mL, 4.00 eq) and K₂OsO₄·2H₂O (21.4 mg, 58.1 μmol, 0.0100 eq). The reaction mixture was stirred at RT for 4 h. The reaction mixture was quenched by addition of saturated aqueous Na₂SO₃ (60.0 mL) at 0° C., and then diluted with H₂O (60.0 mL) and extracted with EtOAc (60.0 mL×3). The combined organic layers were washed with brine (80.0 mL), dried over Na₂SO₄, filtered, and concentrated under reduced pressure to give a residue. The residue was purified by prep-TLC (SiO₂, PE:EtOAc=3:1, R_{f}=0.40). The product, tert-butyl (6-acetylpyridazin-3-yl)carbamate (1.00 g, 4.21 mmol, 73% yield) was obtained as a white solid. ¹H NMR (400 MHz, CDCl₃) δ 8.35 (d, J=9.2 Hz, 1H), 8.12 (d, J=9.2 Hz, 1H), 8.02 (s, 1H), 2.83 (s, 3H), 1.57 (s, 9H). LCMS [M−55]⁺=182.0 m/z.

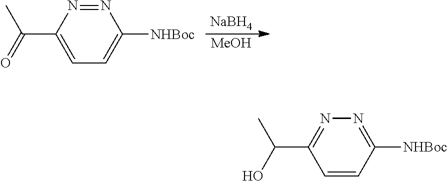 [SMILES: "MeOH" ; CC(=O)c1ccc(NC(=O)OC(C)(C)C)nn1 ; "NaBH4" ; CC(O)c1ccc(NC(=O)OC(C)(C)C)nn1]

To a solution of tert-butyl (6-acetylpyridazin-3-yl)carbamate (1.30 g, 5.48 mmol, 1.00 eq) in MeOH (20.0 mL) was added NaBH₄ (770 mg, 20.3 mmol, 3.71 eq) at 0° C. The reaction mixture was stirred at 0° C. for 2 h. The reaction solution was quenched with 40.0 mL saturated aqueous NH₄Cl solution. The resulting mixture was diluted with H₂O (60.0 mL) and extracted with EtOAc (60.0 mL×3), dried over Na₂SO₄, filtered, and concentrated under reduced pressure to give a residue. The product, tert-butyl (6-(1-hydroxyethyl)pyridazin-3-yl)carbamate (800 mg, 3.34 mmol, 61% yield) was obtained as a yellow oil. LCMS [M−55]⁺=183.9 m/z.

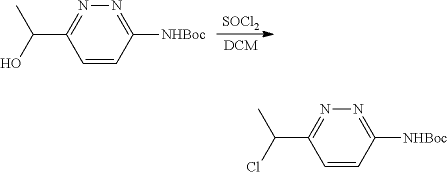 [SMILES: CC(O)c1ccc(NC(=O)OC(C)(C)C)nn1 ; "SOCl2" ; CC(Cl)c1ccc(NC(=O)OC(C)(C)C)nn1]

To a solution of tert-butyl (6-(1-hydroxyethyl)pyridazin-3-yl)carbamate (800 mg, 3.34 mmol, 1.00 eq) in DCM (10.0 mL) was added SOCl₂ (1.19 g, 10.0 mmol, 727 μL, 3.00 eq) at 0° C. The reaction mixture was stirred at RT for 12 h. The reaction mixture was concentrated under reduced pressure to give a residue. The product, tert-butyl (6-(1-chloroethyl)pyridazin-3-yl)carbamate (300 mg, 1.16 mmol, 35% yield), was obtained as a yellow oil. LCMS [M−55]⁺=201.9 m/z.

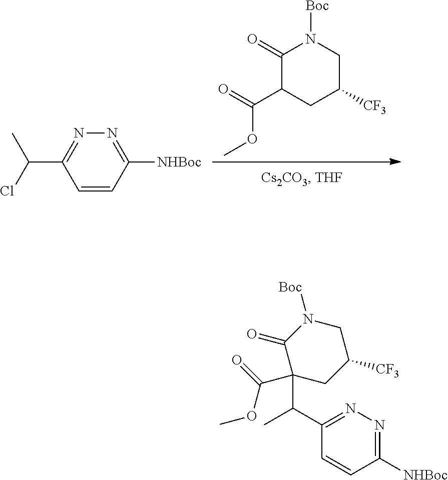 [SMILES: CC(Cl)c1ccc(NC(=O)OC(C)(C)C)nn1 ; COC(=O)C1C[C@@H](C(F)(F)F)CN(C(=O)OC(C)(C)C)C1=O ; COC(=O)C1(C(C)c2ccc(NC(=O)OC(C)(C)C)nn2)C[C@@H](C(F)(F)F)CN(C(=O)OC(C)(C)C)C1=O]

To a solution of 1-(tert-butyl) 3-methyl (5R)-2-oxo-5-(trifluoromethyl)piperidine-1,3-dicarboxylate (347 mg, 1.07 mmol, 1.10 eq) in THF (4.00 mL) was added Cs₂CO₃ (632 mg, 1.94 mmol, 2.00 eq). The reaction mixture was stirred at RT for 30 min. Then tert-butyl (6-(1-chloroethyl)pyridazin-3-yl)carbamate (250 mg, 970 μmol, 1.00 eq) was added and the reaction mixture was stirred at 55° C. for 1.5 h. The reaction mixture was diluted with water (30.0 mL) and extracted with EtOAc (30.0 mL×2). The combined organic layers were washed with brine 30.0 mL, dried over Na₂SO₄, filtered, and concentrated under reduced pressure to give a residue. The product, 1-(tert-butyl) 3-methyl (5R)-3-(1-(6-((tert-butoxycarbonyl)amino)pyridazin-3-yl)ethyl)-2-oxo-5-(trifluoromethyl)piperidine-1,3-dicarboxylate (400 mg, crude), was obtained as a yellow oil. LCMS [M+H]⁺=547.5 m/z.

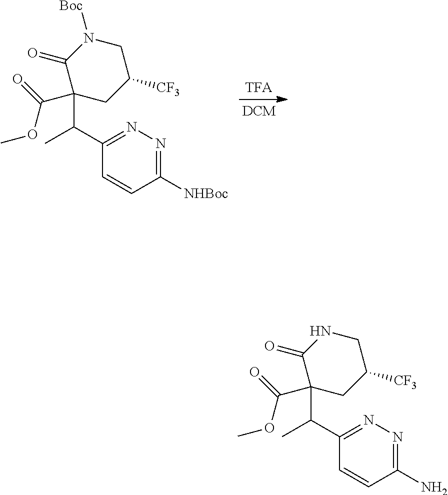 [SMILES: COC(=O)C1(C(C)c2ccc(NC(=O)OC(C)(C)C)nn2)C[C@@H](C(F)(F)F)CN(C(=O)OC(C)(C)C)C1=O ; COC(=O)C1(C(C)c2ccc(N)nn2)C[C@@H](C(F)(F)F)CNC1=O]

To a solution of 1-(tert-butyl) 3-methyl (5R)-3-(1-(6-((tert-butoxycarbonyl)amino)pyridazin-3-yl)ethyl)-2-oxo-5-(trifluoromethyl)piperidine-1,3-dicarboxylate (400 mg, 732 μmol, 1.00 eq) in DCM (2.00 mL) was added TFA (1.03 g, 9.00 mmol, 666 μL, 12.3 eq). The reaction mixture was stirred at RT for 2 h. The reaction mixture was concentrated under reduced pressure to give a residue. Methyl (5R)-3-(1-(6-aminopyridazin-3-yl)ethyl)-2-oxo-5-(trifluoromethyl)piperidine-3-carboxylate (330 mg, 716 μmol, 67% yield over 2 steps, TFA salt) was obtained as a yellow oil. LCMS [M+H]⁺=346.9 m/z.

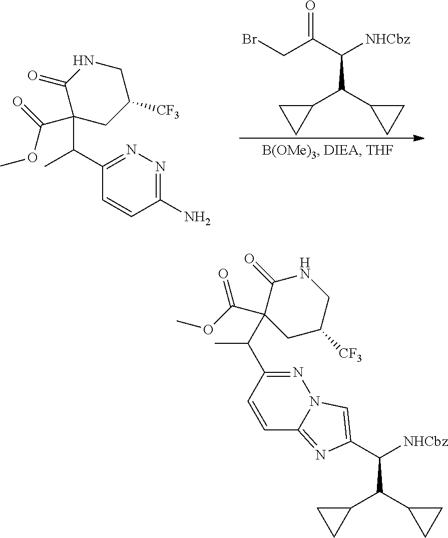 [SMILES: COC(=O)C1(C(C)c2ccc(N)nn2)C[C@@H](C(F)(F)F)CNC1=O ; O=C(N[C@H](C(=O)CBr)C(C1CC1)C1CC1)OCc1ccccc1 ; COC(=O)C1(C(C)c2ccc3nc([C@@H](NC(=O)OCc4ccccc4)C(C4CC4)C4CC4)cn3n2)C[C@@H](C(F)(F)F)CNC1=O]

To a solution of methyl (5R)-3-(1-(6-aminopyridazin-3-yl)ethyl)-2-oxo-5-(trifluoromethyl)piperidine-3-carboxylate (330 mg, 716 μmol, 1.00 eq, TFA salt) and Intermediate 1 (272 mg, 716 μmol, 1.00 eq) in THE (4.00 mL) was added B(OMe)₃ (447 mg, 4.30 mmol, 486 μL, 6.00 eq) and DIEA (556 mg, 4.30 mmol, 749 μL, 6.00 eq). The reaction mixture was stirred at 70° C. for 2 h. The reaction mixture was diluted with water (40.0 mL) and extracted with EtOAc (40.0 mL×2). The combined organic layers were washed with brine (40.0 mL), dried over Na₂SO₄, filtered, and concentrated under reduced pressure to give a residue. The residue was purified by prep-TLC (SiO₂, PE:EtOAc=0:1, R_{f}=0.54). Methyl (5R)-3-(1-(2-((S)-1-(((benzyloxy)carbonyl)amino)-2,2-dicyclopropylethyl)imidazo[1,2-b]pyridazin-6-yl)ethyl)-2-oxo-5-(trifluoromethyl)piperidine-3-carboxylate (230 mg, 366 μmol, 51% yield) was obtained as a yellow oil. LCMS [M+H]⁺=628.5 m/z.

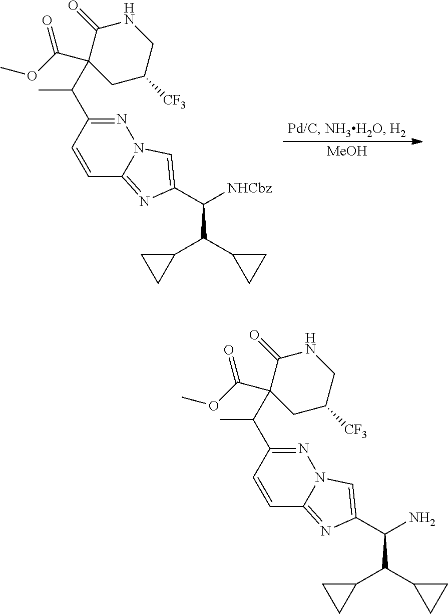 [SMILES: "MeOH" ; COC(=O)C1(C(C)c2ccc3nc([C@@H](NC(=O)OCc4ccccc4)C(C4CC4)C4CC4)cn3n2)C[C@@H](C(F)(F)F)CNC1=O ; "Pd/C, NH3·H2O, H2" ; COC(=O)C1(C(C)c2ccc3nc([C@@H](N)C(C4CC4)C4CC4)cn3n2)C[C@@H](C(F)(F)F)CNC1=O]

Methyl (5R)-3-(1-(2-((S)-1-amino-2,2-dicyclopropylethyl)imidazo[1,2-b]pyridazin-6-yl)ethyl)-2-oxo-5-(trifluoromethyl)piperidine-3-carboxylate was synthesized following General Procedure 3, using methyl (5R)-3-(1-(2-((S)-1-(((benzyloxy)carbonyl)amino)-2,2-dicyclopropylethyl)imidazo[1,2-b]pyridazin-6-yl)ethyl)-2-oxo-5-(trifluoromethyl)piperidine-3-carboxylate, Pd/C (30.0 mg) in MeOH (4.00 mL) and NH₃·H₂O (364 mg, 2.60 mmol, 0.400 mL, 25.0% purity, 7.09 eq), stirring under H₂ for 3 h. The product, methyl (5R)-3-(1-(2-((S)-1-amino-2,2-dicyclopropylethyl)imidazo[1,2-b]pyridazin-6-yl)ethyl)-2-oxo-5-(trifluoromethyl)piperidine-3-carboxylate (170 mg, 344 μmol, 94% yield), was obtained as a yellow oil. LCMS [M+H]⁺=494.0 m/z.

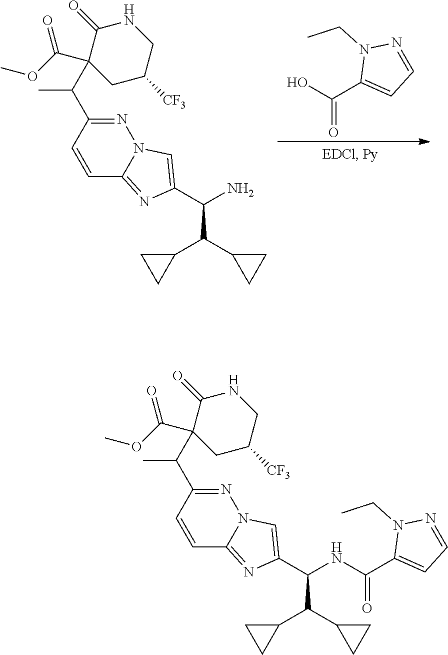

Methyl (5R)-3-(1-(2-((S)-2,2-dicyclopropyl-1-(1-ethyl-1H-pyrazole-5-carboxamido)ethyl)imidazo[1,2-b]pyridazin-6-yl)ethyl)-2-oxo-5-(trifluoromethyl)piperidine-3-carboxylate was synthesized following General Procedure 4, using methyl (5R)-3-(1-(2-((S)-1-amino-2,2-dicyclopropylethyl)imidazo[1,2-b]pyridazin-6-yl)ethyl)-2-oxo-5-(trifluoromethyl)piperidine-3-carboxylate (170 mg, 344 μmol, 1.00 eq), 1-ethyl-1H-pyrazole-5-carboxylic acid (72.4 mg, 516 μmol, 1.50 eq), and EDCI (264 mg, 1.38 mmol, 4.00 eq), stirring for 2 h. The residue was purified by prep-TLC (SiO₂, PE:EtOAc=0:1, R_{f}=0.34). The product, methyl (5R)-3-(1-(2-((S)-2,2-dicyclopropyl-1-(1-ethyl-1H-pyrazole-5-carboxamido)ethyl)imidazo[1,2-b]pyridazin-6-yl)ethyl)-2-oxo-5-(trifluoromethyl)piperidine-3-carboxylate (135 mg, 219 μmol, 64% yield), was obtained as a yellow oil. LCMS [M+H]⁺=616.2 m/Z.

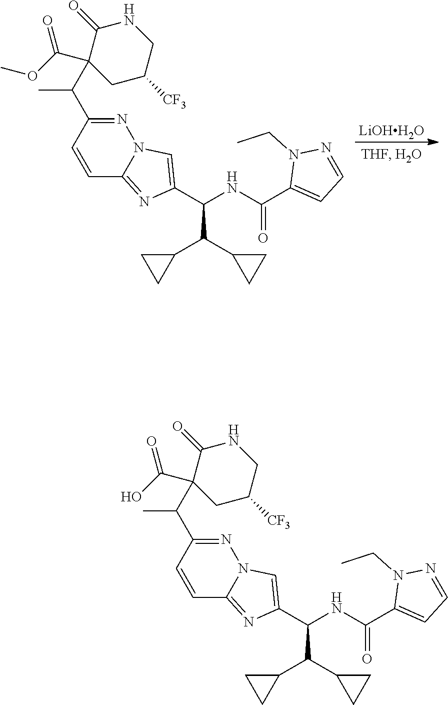

Intermediate (5R)-3-(1-(2-((S)-2,2-dicyclopropyl-1-(1-ethyl-1H-pyrazole-5-carboxamido)ethyl)imidazo[1,2-b]pyridazin-6-yl)ethyl)-2-oxo-5-(trifluoromethyl)piperidine-3-carboxylic acid was synthesized following General Procedure 5, using methyl (5R)-3-(1-(2-((S)-2,2-dicyclopropyl-1-(1-ethyl-1H-pyrazole-5-carboxamido)ethyl)imidazo[1,2-b]pyridazin-6-yl)ethyl)-2-oxo-5-(trifluoromethyl)piperidine-3-carboxylate (135 mg, 219 μmol, 1.00 eq), LiOH·H₂O (46.0 mg, 1.10 mmol, 5.00 eq), stirring for 2 h. The product, (5R)-3-(1-(2-((S)-2,2-dicyclopropyl-1-(1-ethyl-1H-pyrazole-5-carboxamido)ethyl)imidazo[1,2-b]pyridazin-6-yl)ethyl)-2-oxo-5-(trifluoromethyl)piperidine-3-carboxylic acid (120 mg, 199 μmol, 91% yield), was obtained as a white solid. LCMS [M+H]⁺=602.4 m/z.

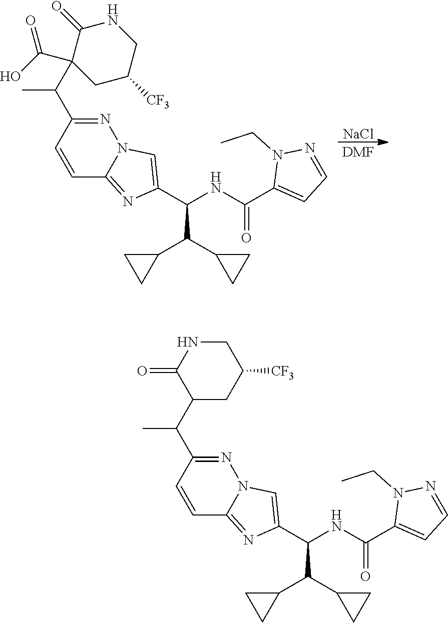

The mixture of stereoisomers, N-((1S)-2,2-dicyclopropyl-1-(6-(1-((5R)-2-oxo-5-(trifluoromethyl)piperidin-3-yl)ethyl)imidazo[1,2-b]pyridazin-2-yl)ethyl)-1-ethyl-1H-pyrazole-5-carboxamide, was synthesized following General Procedure 6, using (5R)-3-(1-(2-((S)-2,2-dicyclopropyl-1-(1-ethyl-1H-pyrazole-5-carboxamido)ethyl)imidazo[1,2-b]pyridazin-6-yl)ethyl)-2-oxo-5-(trifluoromethyl)piperidine-3-carboxylic acid (120 mg, 199 μmol, 1.00 eq), DMF (2.00 mL) and NaCl (34.9 mg, 598 μmol, 3.00 eq), stirring at 110° C. for 30 min. The residue was purified by prep-TLC (SiO₂, PE:EtOAc=0:1, R_{f}=0.43). The mixture of stereoisomers, N-((1S)-2,2-dicyclopropyl-1-(6-(1-((5R)-2-oxo-5-(trifluoromethyl)piperidin-3-yl)ethyl)imidazo[1,2-b]pyridazin-2-yl)ethyl)-1-ethyl-1H-pyrazole-5-carboxamide (100 mg, 167 μmol, 84% yield), was obtained as a white solid. LCMS [M+H]⁺=558.5 m/z.

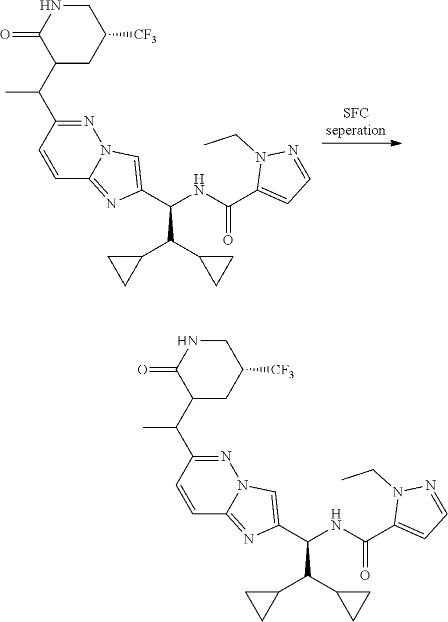 [SMILES: CCn1nccc1C(=O)N[C@H](c1cn2nc(C(C)C3C[C@@H](C(F)(F)F)CNC3=O)ccc2n1)C(C1CC1)C1CC1 ; CCn1nccc1C(=O)N[C@H](c1cn2nc(C(C)C3C[C@@H](C(F)(F)F)CNC3=O)ccc2n1)C(C1CC1)C1CC1]

The mixture of stereoisomers, N-((1S)-2,2-dicyclopropyl-1-(6-(1-((5R)-2-oxo-5-(trifluoromethyl)piperidin-3-yl)ethyl)imidazo[1,2-b]pyridazin-2-yl)ethyl)-1-ethyl-1H-pyrazole-5-carboxamide, was purified by prep-SFC (DAICEL CHIRALCEL OD (250 mm×30 mm, 10 μm); mobile phase: [0.1% NH₃H₂O IPA]; B %: 30%-30%, C8.3; 108 min) and again purified by prep-SFC (DAICEL CHIRALPAK AS (250 mm×30 mm, 10 μm); mobile phase: [0.1% NH₃H₂O MeOH]; B %: 20%-20%, C12; 48 min). The first eluting, single stereoisomer of N-((1S)-2,2-dicyclopropyl-1-(6-(1-((5R)-2-oxo-5-(trifluoromethyl)piperidin-3-yl)ethyl)imidazo[1,2-b]pyridazin-2-yl)ethyl)-1-ethyl-1H-pyrazole-5-carboxamide (Compound 55, 18.87 mg, 33.2 μmol, 20% yield) was obtained as a white solid. LCMS [M+H]⁺=558.5 m/z. The second eluting single stereoisomer of N-((1S)-2,2-dicyclopropyl-1-(6-(1-((5R)-2-oxo-5-(trifluoromethyl)piperidin-3-yl)ethyl)imidazo[1,2-b]pyridazin-2-yl)ethyl)-1-ethyl-1H-pyrazole-5-carboxamide (Compound 56, 6.41 mg, 11.4 μmol, 7.0% yield) was obtained as a white solid. LCMS [M+H]⁺=558.5 m/z. The third eluting, single stereoisomer of N-((1S)-2,2-dicyclopropyl-1-(6-(1-((5R)-2-oxo-5-(trifluoromethyl)piperidin-3-yl)ethyl)imidazo[1,2-b]pyridazin-2-yl)ethyl)-1-ethyl-1H-pyrazole-5-carboxamide (Compound 57, 6.90 mg, 12.2 μmol, 7.5% yield) was obtained as a white solid. LCMS [M+H]⁺=558.5 m/z.

### Example 59: Preparation of Compound 58

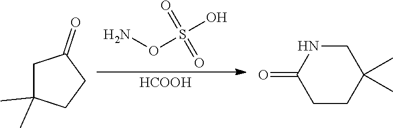

To a solution of 3,3-dimethylcyclopentan-1-one (3.00 g, 26.7 mmol, 1.00 eq) in HCOOH (27.0 mL) was added (aminooxy)sulfonic acid (4.54 g, 40.1 mmol, 1.50 eq). The reaction mixture was stirred at 120° C. for 12 h. The reaction mixture pH was adjusted to 7 with 5 M NaOH. The reaction mixture was partitioned between DCM (100 mL) and H₂O (100 mL). The organic phase was separated, dried over Na₂SO₄, filtered, and concentrated under reduced pressure to give a residue. The residue was purified by column chromatography (SiO₂, PE:EtOAc=5:1 to 1:1). The product, 5,5-dimethylpiperidin-2-one (1.20 g, crude), was obtained as a white solid. ¹H NMR (400 MHz, CDCl₃) δ 6.46 (s, 1H), 2.98-3.02 (m, 2H), 3.26 (t, J=6.8 Hz, 2H), 1.57-1.62 (m, 2H), 1.03 (s, 6H). LCMS [M+H]⁺=128.0 m/z.

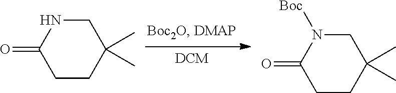

To a solution of 5,5-dimethylpiperidin-2-one (500 mg, crude) in DCM (5.00 mL) was added Boc₂O (2.57 g, 11.7 mmol, 2.71 mL, 3.00 eq) and DMAP (240 mg, 1.97 mmol, 0.50 eq) at 0° C. The reaction mixture was stirred at RT for 2 h. The reaction was concentrated under reduced pressure to give a residue. The residue was purified by column chromatography (SiO₂, PE/EtOAc=5/1 to 3/1). The product, tert-butyl 5,5-dimethyl-2-oxopiperidine-1-carboxylate (600 mg, crude), was obtained as a yellow oil. ¹H NMR (400 MHz, CDCl₃) δ 3.37 (s, 2H), 2.48 (t, J=6.8 Hz, 2H), 1.51 (s, 11H), 1.04 (s, 6H).

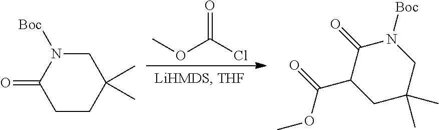

To a mixture of tert-butyl 5,5-dimethyl-2-oxopiperidine-1-carboxylate (400 mg, 1.76 mmol, 1.00 eq) in THF (4.00 mL) was added LiHMDS (1 M, 3.70 mL, 2.10 eq). The mixture was then degassed and purged with N₂ 3 times and stirred at −70° C. for 1 h. Next, there was added methyl chloroformate (831 mg, 8.80 mmol, 681 μL, 5.00 eq) and the reaction mixture was stirred at −70° C. for 1 h under N₂ atmosphere. The reaction mixture was partitioned between EtOAc (60.0 mL) and H₂O (60.0 mL). The organic phase was separated, dried over Na₂SO₄, filtered, and concentrated under reduced pressure to give a residue. The residue was purified by prep-TLC (SiO₂, PE:EtOAc=3:1). The product, 1-(tert-butyl) 3-methyl 5,5-dimethyl-2-oxopiperidine-1,3-dicarboxylate (290 mg, 1.02 mmol, 14% yield over 3 steps), was obtained as a yellow oil. ¹H NMR (400 MHz, CDCl₃) δ 3.77 (s, 3H), 3.47-3.59 (m, 2H), 3.35-3.43 (m, 1H), 1.96-2.08 (m, 1H), 1.79-1.89 (m, 1H), 1.53 (s, 9H), 1.07-1.11 (m, 6H).

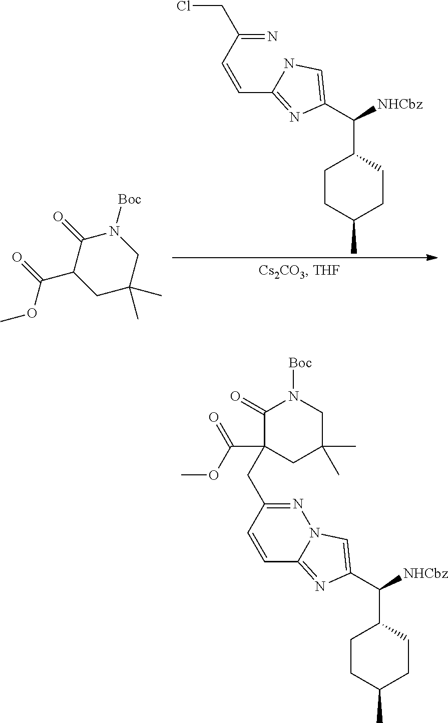

To a solution of 1-(tert-butyl) 3-methyl 5,5-dimethyl-2-oxopiperidine-1,3-dicarboxylate (160 mg, 560 μmol, 1.00 eq) in THF (3.00 mL) was added Cs₂CO₃ (548 mg, 1.68 mmol, 3.00 eq) and benzyl ((S)-(6-(chloromethyl)imidazo[1,2-b]pyridazin-2-yl)((1r,4S)-4-methylcyclohexyl)methyl)carbamate (359 mg, 841 μmol, 1.50 eq). The reaction mixture was stirred at 70° C. for 2 h. The reaction mixture was partitioned between EtOAc (60.0 mL) and H₂O (60.0 mL). The organic phase was separated, dried over Na₂SO₄, filtered, and concentrated under reduced pressure to give a residue. The residue was purified by prep-TLC (SiO₂, PE:EtOAc=1:1). The product, 1-(tert-butyl) 3-methyl 3-((2-((S)-(((benzyloxy)carbonyl)amino)((1r,4S)-4-methylcyclohexyl)methyl)imidazo[1,2-b]pyridazin-6-yl)methyl)-5,5-dimethyl-2-oxopiperidine-1,3-dicarboxylate (170 mg, 251 μmol, 45% yield), was obtained as white solid. LCMS [M+H]⁺=676.5 m/z.

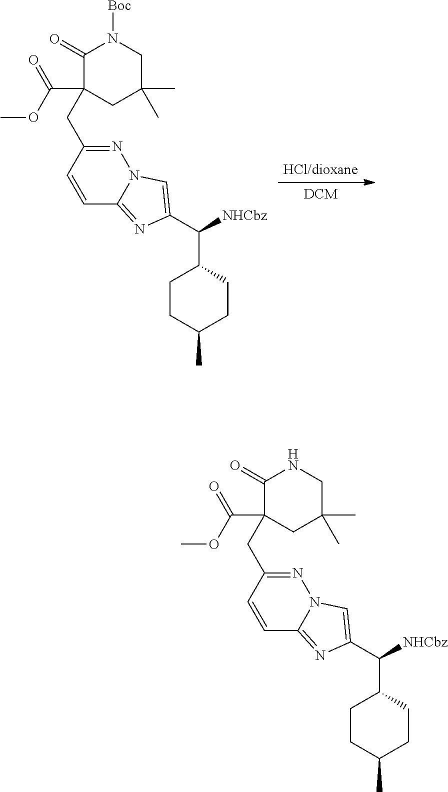 [SMILES: COC(=O)C1(Cc2ccc3nc([C@@H](NC(=O)OCc4ccccc4)[C@H]4CC[C@H](C)CC4)cn3n2)CC(C)(C)CN(C(=O)OC(C)(C)C)C1=O ; COC(=O)C1(Cc2ccc3nc([C@@H](NC(=O)OCc4ccccc4)[C@H]4CC[C@H](C)CC4)cn3n2)CC(C)(C)CNC1=O]

To a solution of 1-(tert-butyl) 3-methyl 3-((2-((S)-(((benzyloxy)carbonyl)amino)((1r,4S)-4-methylcyclohexyl)methyl)imidazo[1,2-b]pyridazin-6-yl)methyl)-5,5-dimethyl-2-oxopiperidine-1,3-dicarboxylate (170 mg, 251 μmol, 1.00 eq) in DCM (2.00 mL) was added HCl/dioxane (4 M, 4.00 mL, 63.6 eq). The reaction mixture was stirred at RT for 30 min. The reaction was concentrated under reduced pressure to give residue. The product, methyl 3-((2-((S)-(((benzyloxy)carbonyl)amino)((1r,4S)-4-methylcyclohexyl)methyl)imidazo[1,2-b]pyridazin-6-yl)methyl)-5,5-dimethyl-2-oxopiperidine-3-carboxylate (140 mg, 228 μmol, 91% yield, HCl salt), was obtained as a yellow solid. LCMS [M+H]⁺=576.3 m/z.

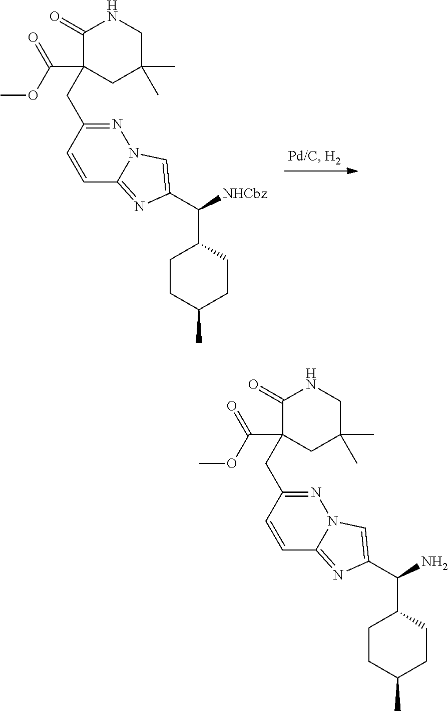 [SMILES: COC(=O)C1(Cc2ccc3nc([C@@H](NC(=O)OCc4ccccc4)[C@H]4CC[C@H](C)CC4)cn3n2)CC(C)(C)CNC1=O ; COC(=O)C1(Cc2ccc3nc([C@@H](N)[C@H]4CC[C@H](C)CC4)cn3n2)CC(C)(C)CNC1=O]

Methyl 3-((2-((S)-amino((1r,4S)-4-methylcyclohexyl)methyl)imidazo[1,2-b]pyridazin-6-yl)methyl)-5,5-dimethyl-2-oxopiperidine-3-carboxylate was synthesized following General Procedure 3, using methyl 3-((2-((S)-(((benzyloxy)carbonyl)amino)((1r,4S)-4-methylcyclohexyl)methyl)imidazo[1,2-b]pyridazin-6-yl)methyl)-5,5-dimethyl-2-oxopiperidine-3-carboxylate (140 mg, 228 μmol, HCl salt), Pd/C (20.0 mg) in 10:1 DCM:MeOH (3.30 mL), stirring under H₂ for 12 h. The product, methyl 3-((2-((S)-amino((1r,4S)-4-methylcyclohexyl)methyl)imidazo[1,2-b]pyridazin-6-yl)methyl)-5,5-dimethyl-2-oxopiperidine-3-carboxylate (90.0 mg, 203 μmol, 89% yield), was obtained as a white solid. LCMS [M+H]⁺=442.4 m/z.

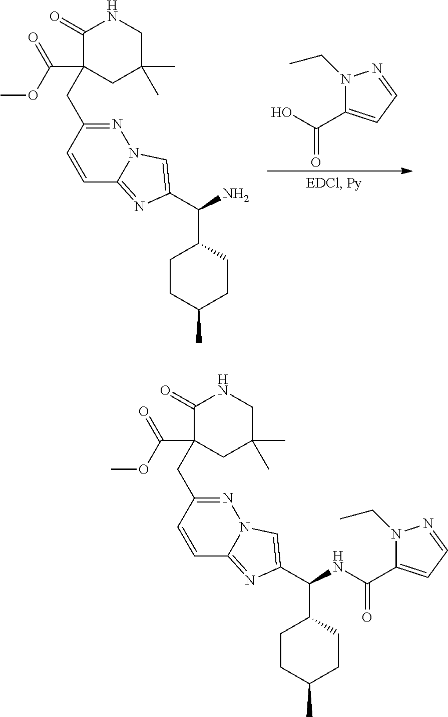

Methyl 3-((2-((S)-(1-ethyl-1H-pyrazole-5-carboxamido)((1r,4S)-4-methylcyclohexyl)methyl)imidazo[1,2-b]pyridazin-6-yl)methyl)-5,5-dimethyl-2-oxopiperidine-3-carboxylate was synthesized following General Procedure 4, using methyl 3-((2-((S)-amino((1r,4S)-4-methylcyclohexyl)methyl)imidazo[1,2-b]pyridazin-6-yl)methyl)-5,5-dimethyl-2-oxopiperidine-3-carboxylate (90.0 mg, 203 μmol, 1.00 eq), 1-ethyl-1H-pyrazole-5-carboxylic acid (42.8 mg, 305 μmol, 1.50 eq), pyridine (2.00 mL), and EDCI (117 mg, 611 μmol, 3.00 eq), stirring for 3 h. The residue was purified by prep-TLC (SiO₂, DCM:MeOH=10:1). The product, methyl 3-((2-((S)-(1-ethyl-1H-pyrazole-5-carboxamido)((1r,4S)-4-methylcyclohexyl)methyl)imidazo[1,2-b]pyridazin-6-yl)methyl)-5,5-dimethyl-2-oxopiperidine-3-carboxylate (70.0 mg, 124 μmol, 61% yield), was obtained as a yellow solid. LCMS [M+H]⁺=564.3 m/z.

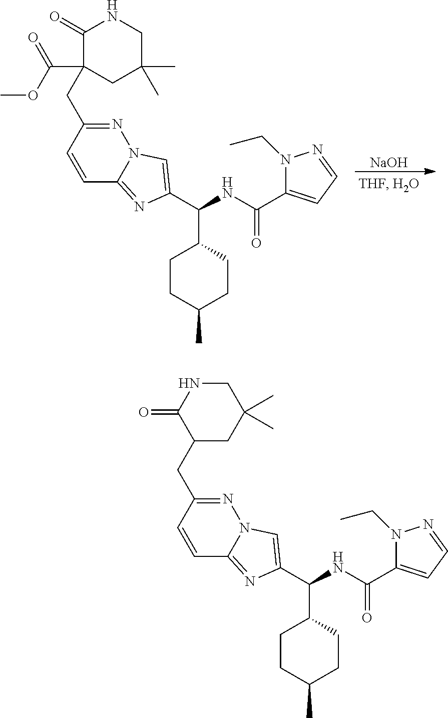

To a solution of methyl 3-((2-((S)-(1-ethyl-1H-pyrazole-5-carboxamido)((1r,4S)-4-methylcyclohexyl)methyl)imidazo[1,2-b]pyridazin-6-yl)methyl)-5,5-dimethyl-2-oxopiperidine-3-carboxylate (70.0 mg, 124 μmol, 1.00 eq) in THF (2.00 mL) was added NaOH (24.8 mg, 620 μmol, 5.00 eq) and H₂O (0.5 mL) at 0° C. The reaction mixture was stirred at 60° C. for 2 h. The reaction mixture was partitioned between EtOAc (60.0 mL) and H₂O (60.0 mL). The organic phase was separated, dried over Na₂SO₄, filtered, and concentrated under reduced pressure to give a residue. The residue was purified by prep-TLC (SiO₂, DCM:MeOH=10:1). The title compound as a mixture of stereoisomers, N-((1S)-(6-((5,5-dimethyl-2-oxopiperidin-3-yl)methyl)imidazo[1,2-b]pyridazin-2-yl)((1r,4S)-4-methylcyclohexyl)methyl)-1-ethyl-1H-pyrazole-5-carboxamide (Compound 58, 39.77 mg, 74.4 μmol, 60% yield), was obtained as a white solid. LCMS [M+H]⁺=506.3 m/z.

### Example 60: Preparation of Compound 59

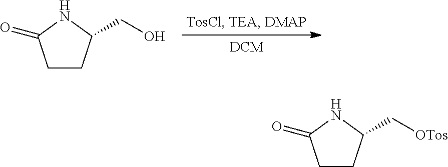

To a solution of (S)-5-(hydroxymethyl)pyrrolidin-2-one (10.0 g, 86.8 mmol, 1.00 eq) and TEA (17.6 g, 173 mmol, 24.1 mL, 2.00 eq) in DCM (200 mL) at RT under N₂ were added DMAP (1.06 g, 8.69 mmol, 0.100 eq) and TosCl (19.8 g, 104 mmol, 1.20 eq). The reaction mixture was then stirred for 15 h at RT under N₂. The reaction mixture was diluted with H₂O (30.0 mL) and extracted with DCM (80.0 mL×3). The combined organic layers were washed with H₂O (100 mL×3) followed by brine (100 mL), dried over Na₂SO₄, filtered, and concentrated under reduced pressure to give a residue. The crude product was purified by reversed phase HPLC (0.1% FA condition) to afford (S)-(5-oxopyrrolidin-2-yl)methyl 4-methylbenzenesulfonate (14.1 g, 52.3 mmol, 60% yield) as a white solid. ¹H NMR (400 MHz, CDCl₃) δ 7.82-7.79 (m, 2H), 7.45 (d, J=8.4 Hz, 2H), 4.08-3.93 (m, 2H), 3.90-3.86 (m, 1H), 2.46 (s, 3H), 2.32-2.20 (m, 3H), 1.84-1.78 (m, 1H). LCMS [M+H]⁺=270.0 m/z.

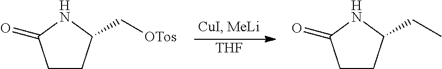

To a solution of CuI (10.6 g, 55.7 mmol, 3.00 eq) in THF (80.0 mL) was added MeLi (1.60 M, 69.6 mL, 6.00 eq) dropwise slowly at 0° C. under N₂, the reaction mixture was stirred at 0° C. for 30 min, then cooled to −20° C. A solution of (S)-(5-oxopyrrolidin-2-yl)methyl 4-methylbenzenesulfonate (5.00 g, 18.57 mmol, 1 eq) in THE (80.0 mL) was added portion-wise and the resulting solution was stirred at −20° C. for 30 min, then stirred at RT for 16 h. The reaction mixture was quenched with saturated aqueous NH₄Cl (120 mL) at 0° C., the reaction mixture was stirred for 30 min, then extracted with EtOAc (80.0 mL×3). The combined organic layers were washed with brine (100 mL) and dried over Na₂SO₄, filtered, and concentrated under reduced pressure to afford (R)-5-ethylpyrrolidin-2-one (2.10 g, crude) as yellow oil. ¹H NMR (400 MHz, CDCl₃) δ 7.01 (s, 1H), 3.60-3.54 (m, 1H), 2.34-2.17 (m, 3H), 1.70-1.65 (m, 1H), 1.57-1.44 (m, 2H), 0.92 (t, J=7.2 Hz, 3H). LCMS [M+H]⁺=114.1 m/z.

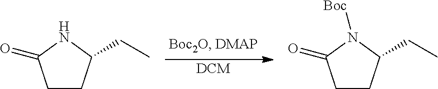

To a solution of (R)-5-ethylpyrrolidin-2-one (1.70 g, crude) in DCM (30.0 mL) was added TEA (3.04 g, 30.0 mmol, 4.18 mL, 2.00 eq) and DMAP (1.84 g, 15.0 mmol, 1.00 eq), then Boc₂O (4.92 g, 22.5 mmol, 5.18 mL, 1.50 eq) was added at 0° C. The reaction mixture was stirred at RT for 3 h. The reaction mixture was diluted with H₂O (6.00 mL) and extracted with DCM (50.0 mL×3). The combined organic layers were washed with H₂O (80.0 mL×2), dried over Na₂SO₄, filtered, and concentrated under reduced pressure to give a residue. The residue was purified by column chromatography (SiO₂, PE/EtOAc=30/1 to 3/1) to afford tert-butyl (R)-2-ethyl-5-oxopyrrolidine-1-carboxylate (2.05 g, 9.61 mmol, 64% yield over 2 steps) as colorless oil. ¹H NMR (400 MHz, CDCl₃) δ 4.08-4.02 (m, 1H), 2.62-2.38 (m, 2H), 2.12-2.04 (m, 1H), 1.85-1.74 (m, 2H), 1.53-1.46 (m, 10H), 0.95-0.90 (m, 3H).

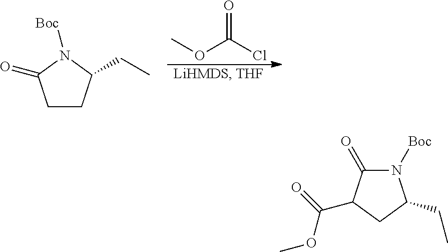

To a solution of tert-butyl (R)-2-ethyl-5-oxopyrrolidine-1-carboxylate (1.70 g, 7.97 mmol, 1.00 eq) in THF (20.0 mL) was added dropwise LiHMDS (1.00 M, 16.0 mL, 2.01 eq) at −70° C. in a 100 mL three-necked flask. After addition, the reaction mixture was stirred at −70° C. for 30 min, and methyl chloroformate (2.62 g, 27.7 mmol, 2.15 mL, 3.48 eq) was added dropwise at −70° C. under N₂. The resulting mixture was stirred at −70° C. for 2 h under N₂. The reaction mixture was quenched with saturated aqueous NH₄Cl (50.0 mL) at 0° C., then the mixture was stirred for 30 min, extracted with EtOAc (50.0 mL×2), and the combined organic layers were washed with saturated aqueous NaHCO₃ (50.0 mL) followed by brine (50.0 mL) and dried over Na₂SO₄, filtered, and concentrated under reduced pressure to give a residue. The residue was purified by column chromatography (SiO₂, PE:EtOAc=30:1 to 5:1), to afford 1-(tert-butyl) 3-methyl (5R)-5-ethyl-2-oxopyrrolidine-1,3-dicarboxylate (2.00 g, 7.37 mmol, 92% yield) as a yellow oil. ¹H NMR (400 MHz, CDCl₃) δ 4.15-3.97 (m, 1H), 3.79 (s, 3H), 3.66-3.52 (m, 1H), 2.57-1.86 (m, 3H), 1.53-1.46 (m, 10H), 0.96-0.90 (m, 3H). LCMS [2M+Na]⁺=565.4 m/z.

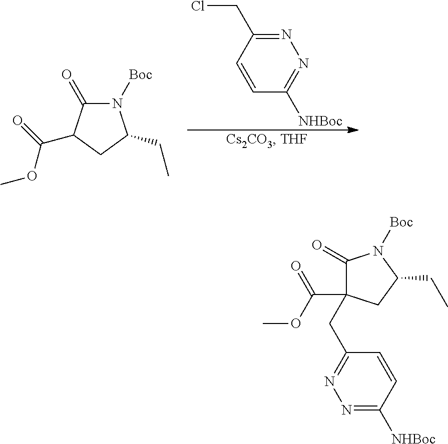

To a solution of 1-(tert-butyl) 3-methyl (5R)-5-ethyl-2-oxopyrrolidine-1,3-dicarboxylate (0.600 g, 2.21 mmol, 1.00 eq) in THF (20.0 mL) was added Cs₂CO₃ (2.88 g, 8.85 mmol, 4.00 eq) and tert-butyl (6-(chloromethyl)pyridazin-3-yl)carbamate (600 mg, 2.46 mmol, 1.11 eq). The reaction mixture was stirred at 55° C. for 5 h. The reaction mixture was diluted with H₂O (30.0 mL) and extracted with EtOAc (30.0 mL×3). The combined organic layers were washed with H₂O (40.0 mL×3) followed by brine (40.0 mL), then dried over Na₂SO₄, filtered, and concentrated under reduced pressure to give a residue. The residue was purified by column chromatography (SiO₂, PE:EtOAc=30:1 to 1:1) to afford 1-(tert-butyl) 3-methyl (5R)-3-((6-((tert-butoxycarbonyl)amino)pyridazin-3-yl)methyl)-5-ethyl-2-oxopyrrolidine-1,3-dicarboxylate (0.820 g, 1.71 mmol, 77% yield) as a white solid. LCMS [M+H]⁺=479.3 m/z.

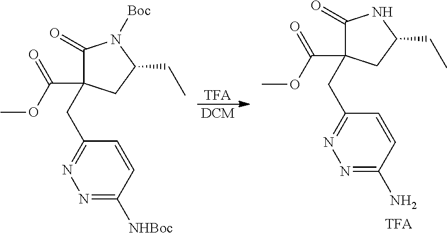

To a solution of 1-(tert-butyl) 3-methyl (5R)-3-((6-((tert-butoxycarbonyl)amino)pyridazin-3-yl)methyl)-5-ethyl-2-oxopyrrolidine-1,3-dicarboxylate (0.820 g, 1.71 mmol, 1.00 eq) in DCM (8.00 mL) was added TFA (6.16 g, 54.0 mmol, 4 mL, 31.5 eq) at 0° C. The reaction mixture was stirred at RT for 3.5 h. The reaction mixture was concentrated under reduced pressure to afford methyl (5R)-3-((6-aminopyridazin-3-yl)methyl)-5-ethyl-2-oxopyrrolidine-3-carboxylate (0.672 g, 1.71 mmol, quantitative, TFA salt) as a yellow oil. LCMS [M+H]⁺=279.1 m/z.

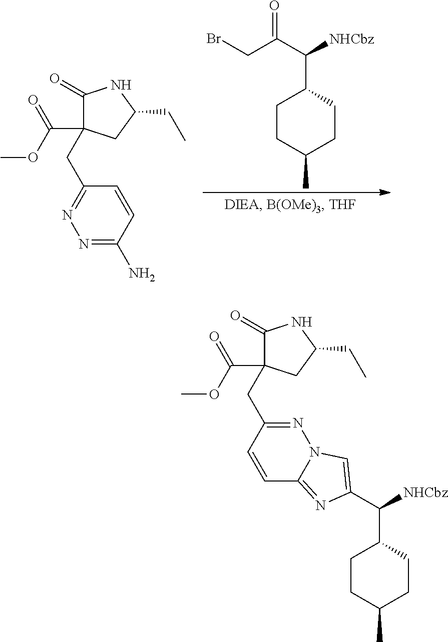 [SMILES: CC[C@@H]1CC(Cc2ccc(N)nn2)(C(=O)OC)C(=O)N1 ; C[C@H]1CC[C@H]([C@H](NC(=O)OCc2ccccc2)C(=O)CBr)CC1 ; CC[C@@H]1CC(Cc2ccc3nc([C@@H](NC(=O)OCc4ccccc4)[C@H]4CC[C@H](C)CC4)cn3n2)(C(=O)OC)C(=O)N1]

Methyl (5R)-3-((2-((S)-(((benzyloxy)carbonyl)amino)((1r,4S)-4-methylcyclohexyl)methyl)imidazo[1,2-b]pyridazin-6-yl)methyl)-5-ethyl-2-oxopyrrolidine-3-carboxylate was synthesized following General Procedure 1, using methyl (5R)-3-((6-aminopyridazin-3-yl)methyl)-5-ethyl-2-oxopyrrolidine-3-carboxylate (0.372 g, 948 μmol, 1.00 eq, TFA salt), Intermediate 4 (435 mg, 1.14 mmol, 1.20 eq), B(OMe)₃ (492 mg, 4.74 mmol, 535 μL, 5.00 eq), and DIEA (1.23 g, 9.48 mmol, 1.65 mL, 10.0 eq). After 4 h at 70° C., crude product was purified by column chromatography (SiO₂, PE:EtOAc=30:1 to 0:1). The product, methyl (5R)-3-((2-((S)-(((benzyloxy)carbonyl)amino)((1r,4S)-4-methylcyclohexyl)methyl)imidazo[1,2-b]pyridazin-6-yl)methyl)-5-ethyl-2-oxopyrrolidine-3-carboxylate (0.490 g, 872 μmol, 92% yield), was obtained as a yellow oil. LCMS [M+H]⁺=562.3 m/z.

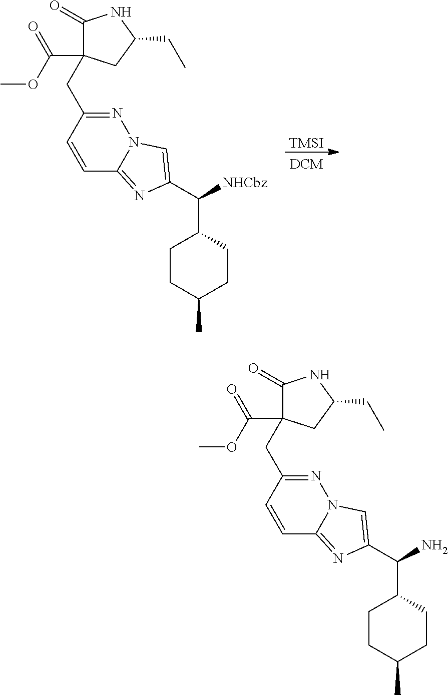 [SMILES: "TMSI" ; CC[C@@H]1CC(Cc2ccc3nc([C@@H](NC(=O)OCc4ccccc4)[C@H]4CC[C@H](C)CC4)cn3n2)(C(=O)OC)C(=O)N1 ; CC[C@@H]1CC(Cc2ccc3nc([C@@H](N)[C@H]4CC[C@H](C)CC4)cn3n2)(C(=O)OC)C(=O)N1]

Methyl (5R)-3-((2-((S)-amino((1r,4S)-4-methylcyclohexyl)methyl)imidazo[1,2-b]pyridazin-6-yl)methyl)-5-ethyl-2-oxopyrrolidine-3-carboxylate was synthesized following General Procedure 2, using methyl (5R)-3-((2-((S)-(((benzyloxy)carbonyl)amino)((1r,4S)-4-methylcyclohexyl)methyl)imidazo[1,2-b]pyridazin-6-yl)methyl)-5-ethyl-2-oxopyrrolidine-3-carboxylate (0.450 g, 801 μmol, 1.00 eq) and TMSI (320 mg, 1.60 mmol, 218 μL, 2.00 eq). The product, methyl (5R)-3-((2-((S)-amino((1r,4S)-4-methylcyclohexyl)methyl)imidazo[1,2-b]pyridazin-6-yl)methyl)-5-ethyl-2-oxopyrrolidine-3-carboxylate (0.280 g, 654 μmol, 82% yield), was obtained as a yellow gum. LCMS [M+H]⁺=428.2 m/z.

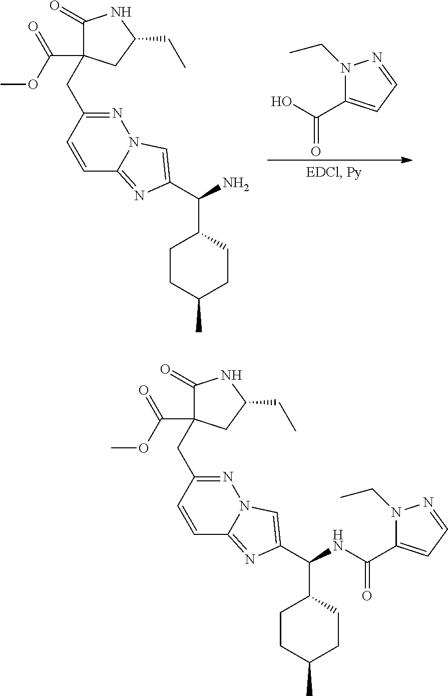

Methyl (5R)-5-ethyl-3-((2-((S)-(1-ethyl-1H-pyrazole-5-carboxamido)((1r,4S)-4-methylcyclohexyl)methyl)imidazo[1,2-b]pyridazin-6-yl)methyl)-2-oxopyrrolidine-3-carboxylate was synthesized following General Procedure 4, using methyl (5R)-3-((2-((S)-amino((1r,4S)-4-methylcyclohexyl)methyl)imidazo[1,2-b]pyridazin-6-yl)methyl)-5-ethyl-2-oxopyrrolidine-3-carboxylate (0.150 g, 350 μmol, 1.00 eq), 1-ethyl-1H-pyrazole-5-carboxylic acid (63.9 mg, 456 μmol, 1.30 eq), and EDCI (202 mg, 1.05 mmol, 3.00 eq), stirring for 2 h. The residue was purified by prep-TLC (SiO₂, PE:EtOAc=0:1). The product, methyl (5R)-5-ethyl-3-((2-((S)-(1-ethyl-1H-pyrazole-5-carboxamido)((1r,4S)-4-methylcyclohexyl)methyl)imidazo[1,2-b]pyridazin-6-yl)methyl)-2-oxopyrrolidine-3-carboxylate (0.135 g, 245.61 μmol, 70% yield), was obtained as a white gum. LCMS [M+H]⁺=550.2 m/z.

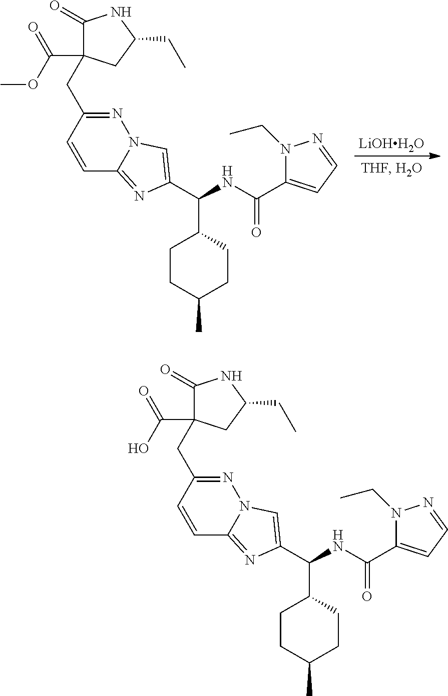

(5R)-5-ethyl-3-((2-((S)-(1-ethyl-1H-pyrazole-5-carboxamido)((1r,4S)-4-methylcyclohexyl)methyl)imidazo[1,2-b]pyridazin-6-yl)methyl)-2-oxopyrrolidine-3-carboxylic acid was synthesized following General Procedure 5, using methyl (5R)-5-ethyl-3-((2-((S)-(1-ethyl-1H-pyrazole-5-carboxamido)((1r,4S)-4-methylcyclohexyl)methyl)imidazo[1,2-b]pyridazin-6-yl)methyl)-2-oxopyrrolidine-3-carboxylate (135 mg, 245 μmol, 1.00 eq), LiOH·H₂O (30.9 mg, 736 μmol, 3.00 eq), stirring for 12 h. The product, (5R)-5-ethyl-3-((2-((S)-(1-ethyl-1H-pyrazole-5-carboxamido)((1r,4S)-4-methylcyclohexyl)methyl)imidazo[1,2-b]pyridazin-6-yl)methyl)-2-oxopyrrolidine-3-carboxylic acid (0.110 g, 205 μmol, 83.6% yield), was obtained as a yellow solid. LCMS [M+H]⁺=536.3 m/z.

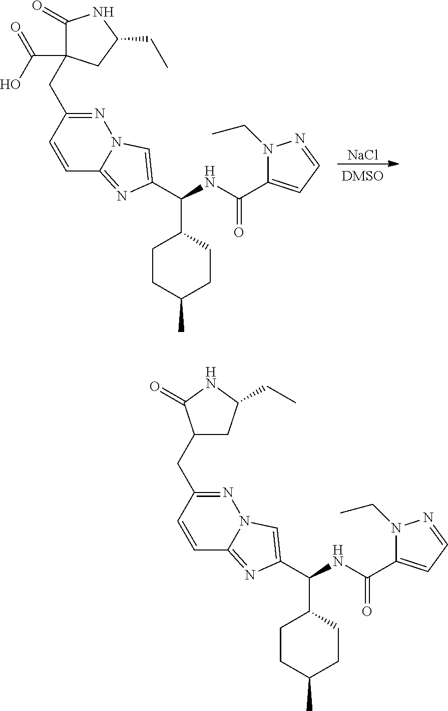

The mixture of stereoisomers, 1-ethyl-N-((1S)-(6-(((5R)-5-ethyl-2-oxopyrrolidin-3-yl)methyl)imidazo[1,2-b]pyridazin-2-yl)((1r,4S)-4-methylcyclohexyl)methyl)-1H-pyrazole-5-carboxamide, was synthesized following General Procedure 6, using (5R)-5-ethyl-3-((2-((S)-(1-ethyl-1H-pyrazole-5-carboxamido)((1r,4S)-4-methylcyclohexyl)methyl)imidazo[1,2-b]pyridazin-6-yl)methyl)-2-oxopyrrolidine-3-carboxylic acid (0.110 g, 205 μmol, 1.00 eq), DMSO (2.00 mL) and NaCl (36.0 mg, 616 μmol, 3.00 eq), stirring at 120° C. for 2 h. The residue was purified by prep-HPLC (Phenomenexluna C18 150×25 mm×10 μm; mobile phase: [water (FA)-ACN]; B %: 43%-73%, 10 min). The mixture of stereoisomers, 1-ethyl-N-((1S)-(6-(((5R)-5-ethyl-2-oxopyrrolidin-3-yl)methyl)imidazo[1,2-b]pyridazin-2-yl)((1r,4S)-4-methylcyclohexyl)methyl)-1H-pyrazole-5-carboxamide (85.0 mg, 172 μmol, 84% yield), was obtained as a yellow oil. LCMS [M+H]⁺=492.3 m/z. The mixture of stereoisomers, 1-ethyl-N-((1S)-(6-(((5R)-5-ethyl-2-oxopyrrolidin-3-yl)methyl)imidazo[1,2-b]pyridazin-2-yl)((1r,4S)-4-methylcyclohexyl)methyl)-1H-pyrazole-5-carboxamide, was separated by SFC (DAICEL CHIRALPAK AS (250 mm×30 mm, 10 μm); 40% mobile phase: [0.1% NH₃H₂O EtOH]). The first eluting stereoisomer of 1-ethyl-N-((1S)-(6-(((5R)-5-ethyl-2-oxopyrrolidin-3-yl)methyl)imidazo[1,2-b]pyridazin-2-yl)((1r,4S)-4-methylcyclohexyl)methyl)-1H-pyrazole-5-carboxamide (Compound 59, 51.47 mg, 104 μmol, 60% yield) was obtained as a white solid. LCMS [M+H]⁺=492.3 m/z.

### Example 61: Preparation of Compound 60

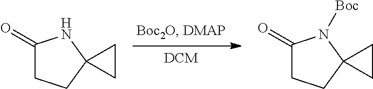 [SMILES: O=C1CCC2(CC2)N1 ; CC(C)(C)OC(=O)N1C(=O)CCC12CC2]

To a solution of 4-azaspiro[2.4]heptan-5-one (400 mg, 3.60 mmol, 1.00 eq) in DCM (5.00 mL) was added Boc₂O (1.57 g, 7.20 mmol, 1.65 mL, 2.00 eq) and DMAP (87.9 mg, 719 μmol, 0.200 eq). The reaction mixture was stirred at RT for 2 h. The reaction mixture was diluted with water (40.0 mL and extracted with DCM (40.0 mL×2). The combined organic layers were washed with brine 40.0 mL), dried over Na₂SO₄, filtered, and concentrated under reduced pressure to give a residue. The product, tert-butyl 5-oxo-4-azaspiro[2.4]heptane-4-carboxylate (800 mg, crude), was obtained as a yellow oil. LCMS [M−55]⁺=156.0 m/z.

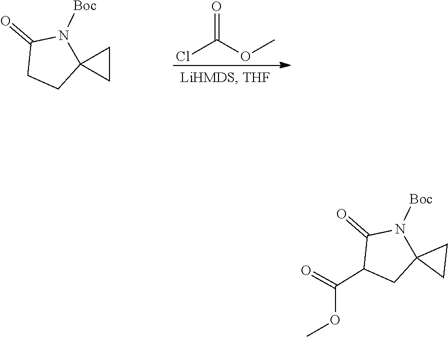

A solution of, tert-butyl 5-oxo-4-azaspiro[2.4]heptane-4-carboxylate (800 mg, crude) in THF (10.0 mL) was added dropwise LiHMDS (1.00 M, 7.57 mL, 2.00 eq) at −70° C., the reaction mixture was stirred at −70° C. for 1 h, then methyl chloroformate (860 mg, 9.10 mmol, 701 μL, 2.40 eq) was added dropwise at −70° C., the reaction mixture was stirred at −70° C. for 1 h. The reaction solution was quenched with 20.0 mL saturated aqueous NH₄Cl solution. The resulting mixture was diluted with H₂O (40.0 mL) and extracted with EtOAc (40.0 mL×3). The combined organic layers were dried over Na₂SO₄, filtered, and concentrated under reduced pressure to give a residue. The residue was purified by prep-TLC (SiO₂, PE:EtOAc=3:1, R_{f}=0.32). The product, 4-(tert-butyl) 6-methyl 5-oxo-4-azaspiro[2.4]heptane-4,6-dicarboxylate (600 mg, 2.23 mmol, 62% yield over 2 steps), was obtained as a yellow oil. LCMS [M−99]⁺=170.0 m/z.

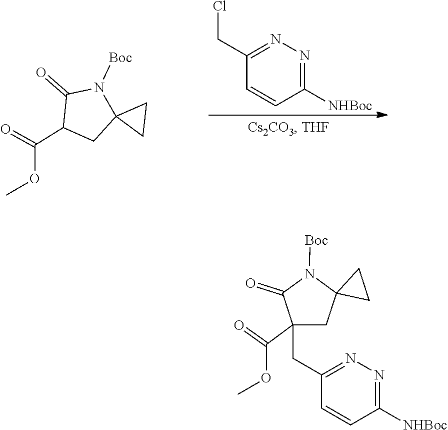 [SMILES: COC(=O)C1CC2(CC2)N(C(=O)OC(C)(C)C)C1=O ; CC(C)(C)OC(=O)Nc1ccc(CCl)nn1 ; COC(=O)C1(Cc2ccc(NC(=O)OC(C)(C)C)nn2)CC2(CC2)N(C(=O)OC(C)(C)C)C1=O]

To a solution of 4-(tert-butyl) 6-methyl 5-oxo-4-azaspiro[2.4]heptane-4,6-dicarboxylate (265 mg, 985 μmol, 1.20 eq) in THE (3.00 mL) was added Cs₂CO₃ (535 mg, 1.64 mmol, 2.00 eq). The reaction mixture was stirred at RT for 30 min. Then tert-butyl (6-(chloromethyl)pyridazin-3-yl)carbamate (200 mg, 821 μmol, 1.00 eq) was added and stirred at 55° C. for 1.5 h. The reaction mixture was diluted with water (40.0 mL) and extracted with EtOAc (40.0 mL×2). The combined organic layers were washed with brine (40.0 mL), dried over Na₂SO₄, filtered, and concentrated under reduced pressure to give a residue. The product, 4-(tert-butyl) 6-methyl 6-((6-((tert-butoxycarbonyl)amino)pyridazin-3-yl)methyl)-5-oxo-4-azaspiro[2.4]heptane-4,6-dicarboxylate (400 mg, crude) was obtained as a yellow oil. LCMS [M+H]⁺=477.4 m/z.

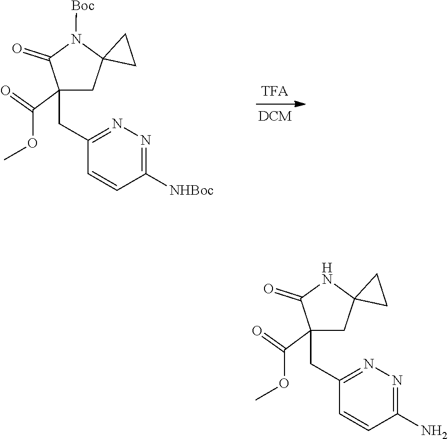 [SMILES: COC(=O)C1(Cc2ccc(NC(=O)OC(C)(C)C)nn2)CC2(CC2)N(C(=O)OC(C)(C)C)C1=O ; COC(=O)C1(Cc2ccc(N)nn2)CC2(CC2)NC1=O]

To a solution of 4-(tert-butyl) 6-methyl 6-((6-((tert-butoxycarbonyl)amino)pyridazin-3-yl)methyl)-5-oxo-4-azaspiro[2.4]heptane-4,6-dicarboxylate (400 mg, crude) in DCM (4.00 mL) was added TFA (1.54 g, 13.5 mmol, 1.00 mL, 16.1 eq). The reaction mixture was stirred at RT for 1 h. The product, methyl 6-((6-aminopyridazin-3-yl)methyl)-5-oxo-4-azaspiro[2.4]heptane-6-carboxylate (350 mg, crude, TFA salt), was obtained as a yellow oil. LCMS [M+H]⁺=277.1 m/z.

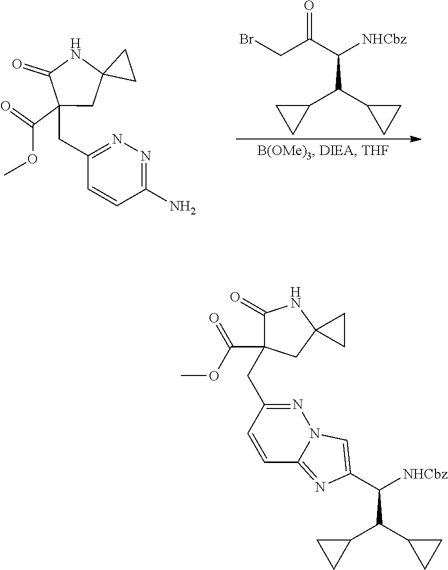 [SMILES: COC(=O)C1(Cc2ccc(N)nn2)CC2(CC2)NC1=O ; O=C(N[C@H](C(=O)CBr)C(C1CC1)C1CC1)OCc1ccccc1 ; COC(=O)C1(Cc2ccc3nc([C@@H](NC(=O)OCc4ccccc4)C(C4CC4)C4CC4)cn3n2)CC2(CC2)NC1=O]

Methyl 6-((2-((S)-1-(((benzyloxy)carbonyl)amino)-2,2-dicyclopropylethyl)imidazo[1,2-b]pyridazin-6-yl)methyl)-5-oxo-4-azaspiro[2.4]heptane-6-carboxylate was synthesized following General Procedure 1, using methyl 6-((6-aminopyridazin-3-yl)methyl)-5-oxo-4-azaspiro[2.4]heptane-6-carboxylate (330 mg, crude, TFA salt), Intermediate 1 (385 mg, 1.01 mmol, 1.20 eq), B(OMe)₃ (527 mg, 5.07 mmol, 573 μL, 6.00 eq), and DIEA (655 mg, 5.07 mmol, 883 μL, 6.00 eq). After 2 h at 70° C., crude product was purified by prep-TLC (SiO₂, PE:EtOAc=0:1, R_{f}=0.40). The product, methyl 6-((2-((S)-1-(((benzyloxy)carbonyl)amino)-2,2-dicyclopropylethyl)imidazo[1,2-b]pyridazin-6-yl)methyl)-5-oxo-4-azaspiro[2.4]heptane-6-carboxylate (280 mg, 502 μmol, 54% yield over 3 steps), was obtained as a yellow solid. LCMS [M+H]⁺=558.5 m/z.

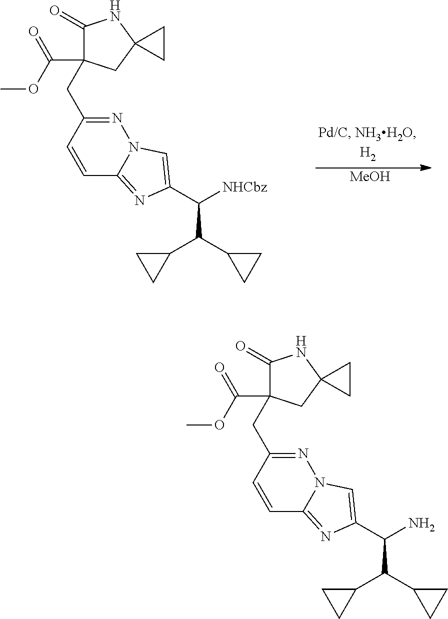 [SMILES: "MeOH" ; COC(=O)C1(Cc2ccc3nc([C@@H](NC(=O)OCc4ccccc4)C(C4CC4)C4CC4)cn3n2)CC2(CC2)NC1=O ; COC(=O)C1(Cc2ccc3nc([C@@H](N)C(C4CC4)C4CC4)cn3n2)CC2(CC2)NC1=O]

Methyl 6-((2-((S)-1-amino-2,2-dicyclopropylethyl)imidazo[1,2-b]pyridazin-6-yl)methyl)-5-oxo-4-azaspiro[2.4]heptane-6-carboxylate was synthesized following General Procedure 3, using methyl 6-((2-((S)-1-(((benzyloxy)carbonyl)amino)-2,2-dicyclopropylethyl)imidazo[1,2-b]pyridazin-6-yl)methyl)-5-oxo-4-azaspiro[2.4]heptane-6-carboxylate (280 mg, 502 μmol, 1.00 eq), Pd/C (50.0 mg) in MeOH and NH₃·H₂O (364 mg, 2.60 mmol, 0.400 mL, 25.0% purity, 5.17 eq), stirring under H₂ for 1 h. The product, methyl 6-((2-((S)-1-amino-2,2-dicyclopropylethyl)imidazo[1,2-b]pyridazin-6-yl)methyl)-5-oxo-4-azaspiro[2.4]heptane-6-carboxylate (200 mg, 472 μmol, 94% yield), was obtained as a yellow oil. LCMS [M+H]⁺=424.4 m/z.

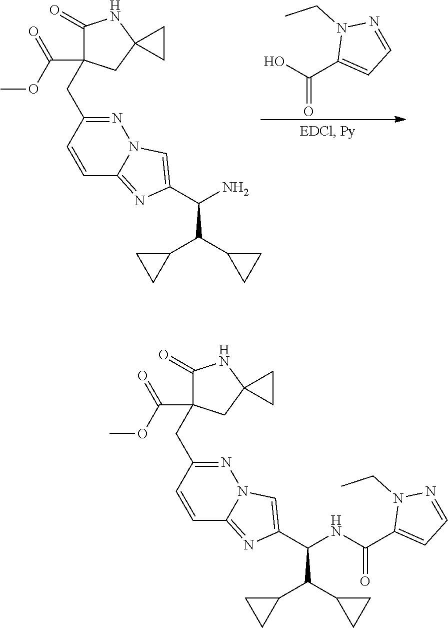

Methyl 6-((2-((S)-2,2-dicyclopropyl-1-(1-ethyl-1H-pyrazole-5-carboxamido)ethyl)imidazo[1,2-b]pyridazin-6-yl)methyl)-5-oxo-4-azaspiro[2.4]heptane-6-carboxylate was synthesized following General Procedure 4, using methyl 6-((2-((S)-1-amino-2,2-dicyclopropylethyl)imidazo[1,2-b]pyridazin-6-yl)methyl)-5-oxo-4-azaspiro[2.4]heptane-6-carboxylate (190 mg, 448 μmol, 1.00 eq), 1-ethyl-1H-pyrazole-5-carboxylic acid (94.3 mg, 673 μmol, 1.50 eq), and EDCI (344 mg, 1.79 mmol, 4.00 eq), stirring for 2 h. The residue was purified by prep-TLC (SiO₂, DCM:MeOH=10:1, R_{f}=0.37). The product, methyl 6-((2-((S)-2,2-dicyclopropyl-1-(1-ethyl-1H-pyrazole-5-carboxamido)ethyl)imidazo[1,2-b]pyridazin-6-yl)methyl)-5-oxo-4-azaspiro[2.4]heptane-6-carboxylate (160 mg, 293 μmol, 65% yield), was obtained as a white solid. LCMS [M+H]⁺=546.5 m/z.

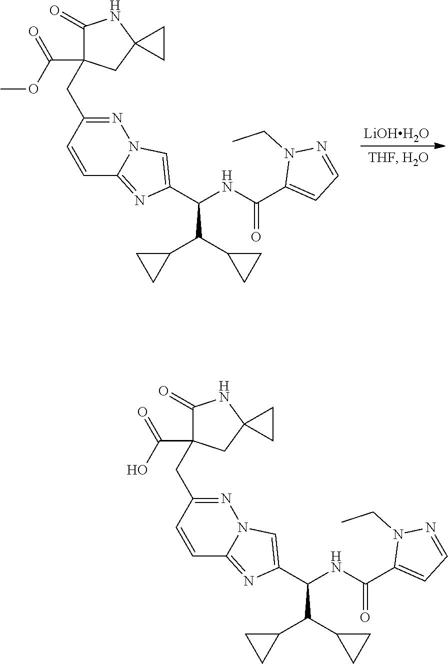

Intermediate 6-((2-((S)-2,2-dicyclopropyl-1-(1-ethyl-1H-pyrazole-5-carboxamido)ethyl)imidazo[1,2-b]pyridazin-6-yl)methyl)-5-oxo-4-azaspiro[2.4]heptane-6-carboxylic acid was synthesized following General Procedure 5, using methyl 6-((2-((S)-2,2-dicyclopropyl-1-(1-ethyl-1H-pyrazole-5-carboxamido)ethyl)imidazo[1,2-b]pyridazin-6-yl)methyl)-5-oxo-4-azaspiro[2.4]heptane-6-carboxylate (150 mg, 275 μmol, 1.00 eq), LiOH·H₂O (46.1 mg, 1.10 mmol, 4.00 eq), stirring for 30 min. The product, 6-((2-((S)-2,2-dicyclopropyl-1-(1-ethyl-1H-pyrazole-5-carboxamido)ethyl)imidazo[1,2-b]pyridazin-6-yl)methyl)-5-oxo-4-azaspiro[2.4]heptane-6-carboxylic acid (140 mg, 263 μmol, 96% yield), was obtained as a yellow oil. LCMS [M+H]⁺=532.2 m/z.

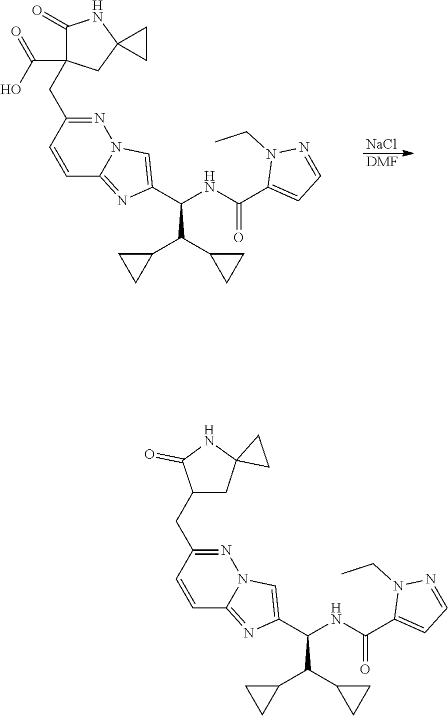

The mixture of stereoisomers, N-((1S)-2,2-dicyclopropyl-1-(6-((5-oxo-4-azaspiro[2.4]heptan-6-yl)methyl)imidazo[1,2-b]pyridazin-2-yl)ethyl)-1-ethyl-1H-pyrazole-5-carboxamide, was synthesized following General Procedure 6, using 6-((2-((S)-2,2-dicyclopropyl-1-(1-ethyl-1H-pyrazole-5-carboxamido)ethyl)imidazo[1,2-b]pyridazin-6-yl)methyl)-5-oxo-4-azaspiro[2.4]heptane-6-carboxylic acid (130 mg, 244 μmol, 1.00 eq), DMF (4.00 mL) and NaCl 42.8 mg, 733 μmol, 3.00 eq), stirring at 110° C. for 30 min. The residue was purified by prep-TLC (SiO₂, DCM:MeOH=10:1, R_{f}=0.35). The mixture of stereoisomers, N-((1S)-2,2-dicyclopropyl-1-(6-((5-oxo-4-azaspiro[2.4]heptan-6-yl)methyl)imidazo[1,2-b]pyridazin-2-yl)ethyl)-1-ethyl-1H-pyrazole-5-carboxamide (100 mg, 205 μmol, 84% yield), was obtained as a yellow oil. LCMS [M+H]⁺=488.2 m/z. Purification by prep-SFC (Venusil ASB Phenyl 150×30 mm×5 um; mobile phase: [Neu-MeOH]; B %: 50%-50%, A4.8; 30 min) afforded the first single eluting stereoisomer of N-((1S)-2,2-dicyclopropyl-1-(6-((5-oxo-4-azaspiro[2.4]heptan-6-yl)methyl)imidazo[1,2-b]pyridazin-2-yl)ethyl)-1-ethyl-1H-pyrazole-5-carboxamide (Compound 60, 19.45 mg, 39.6 μmol, 19% yield) as a white solid. LCMS [M+H]⁺=488.3 m/z.

### Example 62: Preparation of Compound 61

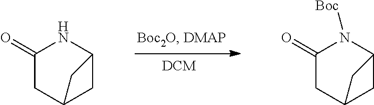 [SMILES: O=C1CC2CC(C2)N1 ; CC(C)(C)OC(=O)N1C(=O)CC2CC1C2]

To a solution of 2-azabicyclo[3.1.1]heptan-3-one (550 mg, 4.95 mmol, 1.00 eq) in DCM (3.00 mL) was added (Boc)₂O (1.62 g, 7.42 mmol, 1.71 mL, 1.50 eq) and DMAP (120 mg, 989 μmol, 0.200 eq). The reaction mixture was stirred at RT for 12 h. The reaction mixture was diluted with H₂O (30.0 mL) and extracted with EtOAc (30.0 mL×3). The combined organic layers were washed with saturated aqueous NH₄Cl (30.0 mL×2), dried over Na₂SO₄, filtered, and concentrated under reduced pressure to give a residue. The residue was purified by column chromatography (SiO₂, PE/EtOAc=100/1 to 10/1). The product, tert-butyl 3-oxo-2-azabicyclo[3.1.1]heptane-2-carboxylate (900 mg, 4.26 mmol, 86% yield), was obtained as a white solid. LCMS [2M+Na]⁺=445.2 m/z.

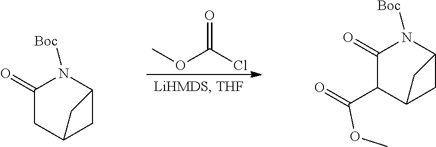

To a solution of, tert-butyl 3-oxo-2-azabicyclo[3.1.1]heptane-2-carboxylate (900 mg, 4.26 mmol, 1.00 eq) in THF (5.00 mL) was added LiHMDS (1 M, 8.52 mL, 2.00 eq) at −70° C. for 30 min. Methyl chloroformate (603 mg, 6.39 mmol, 494 μL, 1.50 eq) was then added at −70° C. The reaction mixture was stirred at −70° C. for 1 h. The reaction mixture was diluted with H₂O (30.0 mL) and extracted with EtOAc (30.0 mL×3). The combined organic layers were washed with saturated aqueous NaHCO₃ (30.0 mL×2), dried over Na₂SO₄, filtered, and concentrated under reduced pressure to give a residue. The residue was purified by column chromatography (SiO₂, PE:EtOAc=100:1 to 10:1. The product, 2-(tert-butyl) 4-methyl 3-oxo-2-azabicyclo[3.1.1]heptane-2,4-dicarboxylate (0.900 g, 3.34 mmol, 78% yield), was obtained as a white solid. LCMS [2M+Na]⁺=561.2 m/z.

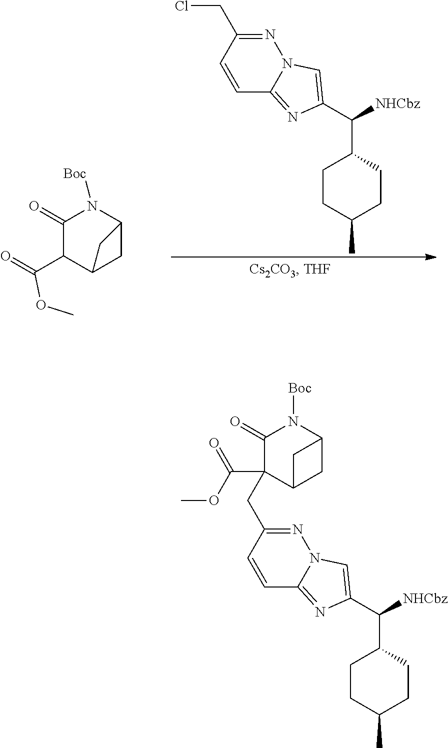 [SMILES: COC(=O)C1C(=O)N(C(=O)OC(C)(C)C)C2CC1C2 ; C[C@H]1CC[C@H]([C@H](NC(=O)OCc2ccccc2)c2cn3nc(CCl)ccc3n2)CC1 ; COC(=O)C1(Cc2ccc3nc([C@@H](NC(=O)OCc4ccccc4)[C@H]4CC[C@H](C)CC4)cn3n2)C(=O)N(C(=O)OC(C)(C)C)C2CC1C2]

To a solution of 2-(tert-butyl) 4-methyl 3-oxo-2-azabicyclo[3.1.1]heptane-2,4-dicarboxylate (180 mg, 668 μmol, 1.00 eq) in THF (2.00 mL) was added benzyl ((S)-(6-(chloromethyl)imidazo[1,2-b]pyridazin-2-yl)((1r,4S)-4-methylcyclohexyl)methyl)carbamate (342 mg, 802 μmol, 1.20 eq) and Cs₂CO₃ (1.31 g, 4.01 mmol, 6.00 eq). The reaction mixture was stirred at 70° C. for 12 h. The reaction mixture was diluted with H₂O (30.0 mL) and extracted with EtOAc (30.0 mL×3). The combined organic layers were washed with saturated aqueous NaHCO₃ (30.0 mL×2), dried over Na₂SO₄, filtered, and concentrated under reduced pressure to give a residue. The residue was purified by prep-TLC (SiO₂, PE:EtOAc=1:1). The product, 2-(tert-butyl) 4-methyl 4-((2-((S)-(((benzyloxy)carbonyl)amino)((1r,4S)-4-methylcyclohexyl)methyl)imidazo[1,2-b]pyridazin-6-yl)methyl)-3-oxo-2-azabicyclo[3.1.1]heptane-2,4-dicarboxylate (173 mg, 262 μmol, 39% yield) was obtained as a white solid. LCMS [M+H]⁺=660.3 m/z.

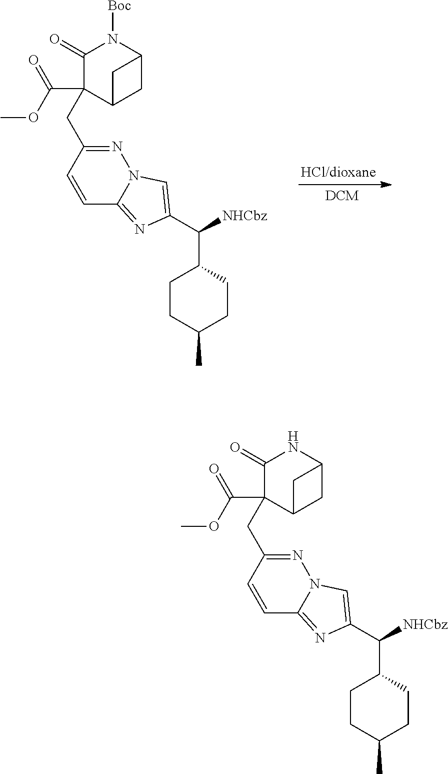 [SMILES: COC(=O)C1(Cc2ccc3nc([C@@H](NC(=O)OCc4ccccc4)[C@H]4CC[C@H](C)CC4)cn3n2)C(=O)N(C(=O)OC(C)(C)C)C2CC1C2 ; COC(=O)C1(Cc2ccc3nc([C@@H](NC(=O)OCc4ccccc4)[C@H]4CC[C@H](C)CC4)cn3n2)C(=O)NC2CC1C2]

To a solution of 2-(tert-butyl) 4-methyl 4-((2-((S)-(((benzyloxy)carbonyl)amino)((1r,4 S)-4-methylcyclohexyl)methyl)imidazo[1,2-b]pyridazin-6-yl)methyl)-3-oxo-2-azabicyclo[3.1.1]heptane-2,4-dicarboxylate (170 mg, 257 μmol, 1.00 eq) in DCM (2.00 mL) was added HCl/dioxane (4 M, 1.29 mL, 20.0 eq) at 0° C. The reaction mixture was stirred at RT for 1 h. The reaction mixture was concentrated under reduced pressure to give a residue. The product, methyl 4-((2-((S)-(((benzyloxy)carbonyl)amino)((1r,4S)-4-methylcyclohexyl)methyl)imidazo[1,2-b]pyridazin-6-yl)methyl)-3-oxo-2-azabicyclo[3.1.1]heptane-4-carboxylate (140 mg, 234 μmol, 91% yield, HCl salt) was obtained as a white solid. LCMS [M+H]⁺=560.3 m/z.

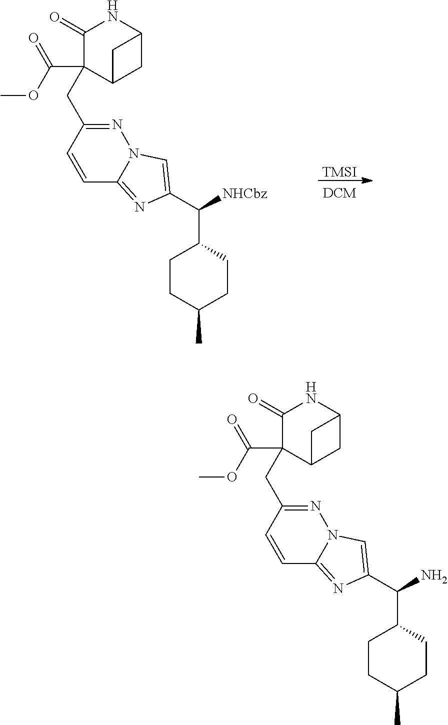 [SMILES: "TMSI" ; COC(=O)C1(Cc2ccc3nc([C@@H](NC(=O)OCc4ccccc4)[C@H]4CC[C@H](C)CC4)cn3n2)C(=O)NC2CC1C2 ; COC(=O)C1(Cc2ccc3nc([C@@H](N)[C@H]4CC[C@H](C)CC4)cn3n2)C(=O)NC2CC1C2]

Methyl 4-((2-((S)-amino((1r,4S)-4-methylcyclohexyl)methyl)imidazo[1,2-b]pyridazin-6-yl)methyl)-3-oxo-2-azabicyclo[3.1.1]heptane-4-carboxylate was synthesized following General Procedure 2, using methyl 4-((2-((S)-(((benzyloxy)carbonyl)amino)((1r,4S)-4-methylcyclohexyl)methyl)imidazo[1,2-b]pyridazin-6-yl)methyl)-3-oxo-2-azabicyclo[3.1.1]heptane-4-carboxylate (140 mg, 234 μmol, 1.00 eq, HCl salt) and TMSI (93.9 mg, 469 μmol, 63.9 μL, 2.00 eq). The product, methyl 4-((2-((S)-amino((1r,4S)-4-methylcyclohexyl)methyl)imidazo[1,2-b]pyridazin-6-yl)methyl)-3-oxo-2-azabicyclo[3.1.1]heptane-4-carboxylate (80.0 mg, 188 μmol, 80% yield), was obtained as a yellow solid. LCMS [M+H]⁺=426.2 m/z.

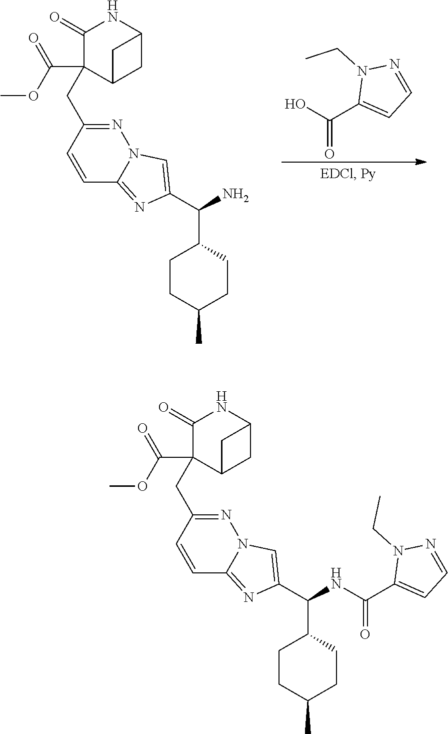

Methyl 4-((2-((S)-(1-ethyl-1H-pyrazole-5-carboxamido)((1r,4S)-4-methylcyclohexyl)methyl)imidazo[1,2-b]pyridazin-6-yl)methyl)-3-oxo-2-azabicyclo[3.1.1]heptane-4-carboxylate was synthesized following General Procedure 4, using methyl 4-((2-((S)-amino((1r,4S)-4-methylcyclohexyl)methyl)imidazo[1,2-b]pyridazin-6-yl)methyl)-3-oxo-2-azabicyclo[3.1.1]heptane-4-carboxylate (50.0 mg, 117 μmol, 1.00 eq), 1-ethyl-1H-pyrazole-5-carboxylic acid (19.7 mg, 141 μmol, 1.20 eq), and EDCI (67.5 mg, 352 μmol, 3.00 eq), stirring for 2 h. The residue was purified by prep-TLC (SiO₂, DCM:MeOH=8:1). The product, methyl 4-((2-((S)-(1-ethyl-1H-pyrazole-5-carboxamido)((1r,4S)-4-methylcyclohexyl)methyl)imidazo[1,2-b]pyridazin-6-yl)methyl)-3-oxo-2-azabicyclo[3.1.1]heptane-4-carboxylate (50.0 mg, 91.3 μmol, 78% yield), was obtained as a yellow solid, which was used in the next step without further purification.

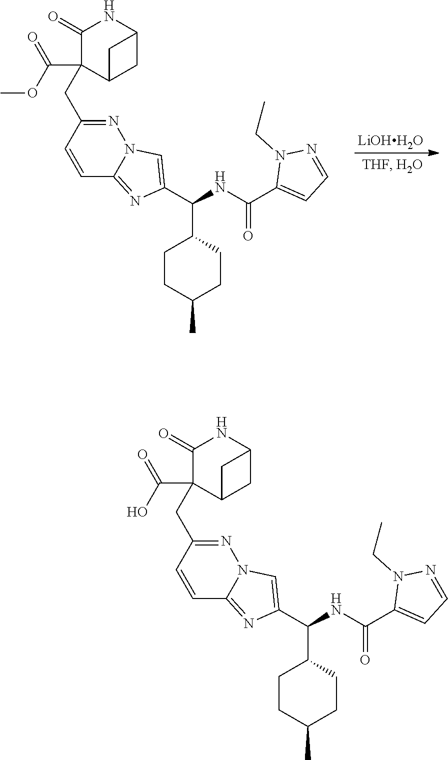

Intermediate 4-((2-((S)-(1-ethyl-1H-pyrazole-5-carboxamido)((1r,4S)-4-methylcyclohexyl)methyl)imidazo[1,2-b]pyridazin-6-yl)methyl)-3-oxo-2-azabicyclo[3.1.1]heptane-4-carboxylic acid was synthesized following General Procedure 5, using methyl 4-((2-((S)-(1-ethyl-1H-pyrazole-5-carboxamido)((1r,4S)-4-methylcyclohexyl)methyl)imidazo[1,2-b]pyridazin-6-yl)methyl)-3-oxo-2-azabicyclo[3.1.1]heptane-4-carboxylate (50.0 mg, 91.3 μmol, 1.00 eq), LiOH·H₂O (11.4 mg 273 μmol, 3.00 eq), stirring for 1 h. The product, 4-((2-((S)-(1-ethyl-1H-pyrazole-5-carboxamido)((1r,4S)-4-methylcyclohexyl)methyl)imidazo[1,2-b]pyridazin-6-yl)methyl)-3-oxo-2-azabicyclo[3.1.1]heptane-4-carboxylic acid (45.0 mg, 84.3 μmol, 92% yield), was obtained as a white solid. LCMS [M+H]⁺=534.3 m/z.

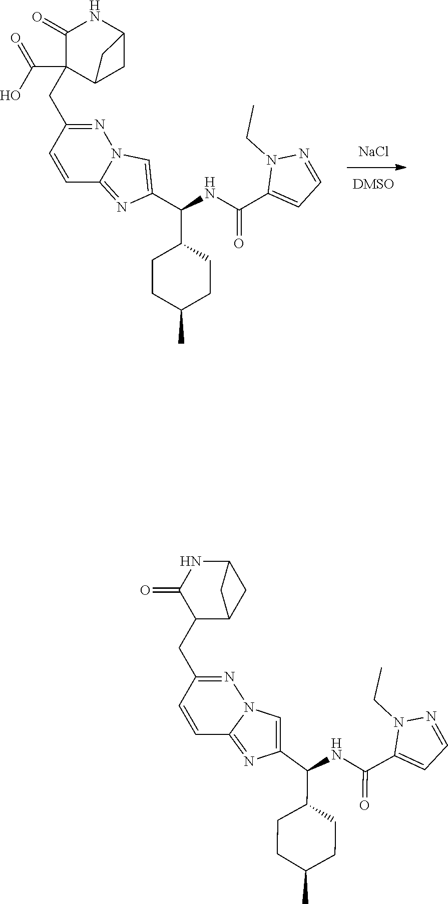

The mixture of stereoisomers, 1-ethyl-N-((1S)-((1r,4S)-4-methylcyclohexyl)(6-((3-oxo-2-azabicyclo[3.1.1]heptan-4-yl)methyl)imidazo[1,2-b]pyridazin-2-yl)methyl)-1H-pyrazole-5-carboxamide, was synthesized following General Procedure 6, using 4-((2-((S)-(1-ethyl-1H-pyrazole-5-carboxamido)((1r,4S)-4-methylcyclohexyl)methyl)imidazo[1,2-b]pyridazin-6-yl)methyl)-3-oxo-2-azabicyclo[3.1.1]heptane-4-carboxylic acid (45.0 mg, 84.3 μmol, 1.00 eq), DMSO (3.00 mL) and NaCl (24.6 mg, 421 μmol, 5.00 eq), stirring at 100° C. for 2 h. The residue was purified by prep-TLC (SiO₂, DCM:MeOH=8:1). The mixture of stereoisomers, 1-ethyl-N-((1S)-((1r,4S)-4-methylcyclohexyl)(6-((3-oxo-2-azabicyclo[3.1.1]heptan-4-yl)methyl)imidazo[1,2-b]pyridazin-2-yl)methyl)-1H-pyrazole-5-carboxamide (16.3 mg, 31.4 μmol, 37.2% yield), was obtained as a white solid. LCMS [M+H]⁺=490.3 m/z. The mixture of stereoisomers, 1-ethyl-N-((1S)-((1r,4S)-4-methylcyclohexyl)(6-((3-oxo-2-azabicyclo[3.1.1]heptan-4-yl)methyl)imidazo[1,2-b]pyridazin-2-yl)methyl)-1H-pyrazole-5-carboxamide, was separated by prep-SFC (DAICEL CHIRALPAK AD (250 mm×30 mm, 10 μm); mobile phase: [0.1% NH₃H₂O EtOH]; B %: 40%-40%, 3.4 min). The second eluting stereoisomer of 1-ethyl-N-((1S)-((1r,4S)-4-methylcyclohexyl)(6-((3-oxo-2-azabicyclo[3.1.1]heptan-4-yl)methyl)imidazo[1,2-b]pyridazin-2-yl)methyl)-1H-pyrazole-5-carboxamide (Compound 61, 14.73 mg, 28.6 μmol, 35% yield) was obtained as a white solid. LCMS [M+H]⁺=490.2 m/z.

### Example 63: Preparation of Compound 62

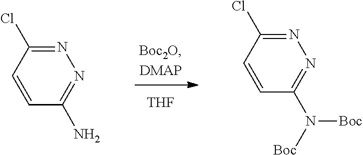

To a solution of 6-chloropyridazin-3-amine (15.0 g, 115 mmol, 1.00 eq) in THF (200 mL) was added Boc₂O (25.2 g, 115 mmol, 26.6 mL, 1.00 eq) and DMAP (2.12 g, 17.3 mmol, 0.15 eq) at 0° C. The reaction mixture was stirred at RT for 2 h. The reaction mixture was concentrated under reduced pressure to remove THF. The residue was purified by column chromatography (SiO₂, PE:EtOAc=1:0 to 1:1). The product, tert-butyl (tert-butoxycarbonyl)(6-chloropyridazin-3-yl)carbamate (22.0 g, 66.7 mmol, 58% yield), was obtained as a white solid. ¹H NMR (400 MHz, CDCl₃) δ7.50-7.55 (m, 2H), 1.44-1.51 (m, 18H). LCMS [2M+Na]⁺=681.4 m/z.

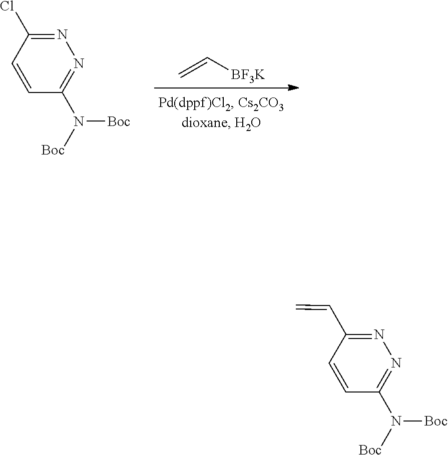

A mixture of tert-butyl (tert-butoxycarbonyl)(6-chloropyridazin-3-yl)carbamate (17.0 g 51.5 mmol, 1.00 eq), potassium vinyltrifluoroborate (34.5 g, 257 mmol, 5.00 eq), Pd(dppf)Cl₂ (1.90 g, 5.15 mmol, 0.10 eq), and Cs₂CO₃ (33.5 g, 103 mmol, 2.00 eq) in dioxane (200 mL) and H₂O (20.0 mL) was degassed and purged with N₂ 3 times, and then the reaction mixture was stirred at 80° C. for 2 h under N₂ atmosphere. The reaction mixture was partitioned between EtOAc (400 mL) and H₂O (200 mL). The organic phase was separated, dried over Na₂SO₄, filtered, and concentrated under reduced pressure to give a residue. The residue was purified by column chromatography (SiO₂, PE:EtOAc=5:1 to 3:1). The product, tert-butyl (tert-butoxycarbonyl)(6-vinylpyridazin-3-yl)carbamate (12.0 g, 37.3 mmol, 72% yield), was obtained as a light-yellow solid. ¹H NMR (400 MHz, CDCl₃) δ7.62-7.64 (m, 1H), 7.45-7.47 (m, 1H), 7.03-7.10 (m, 1H), 6.22-6.27 (m, 1H), 5.70-5.72 (m, 1H), 1.43-1.50 (m, 18H). LCMS [M+H]⁺=322.1 m/z.

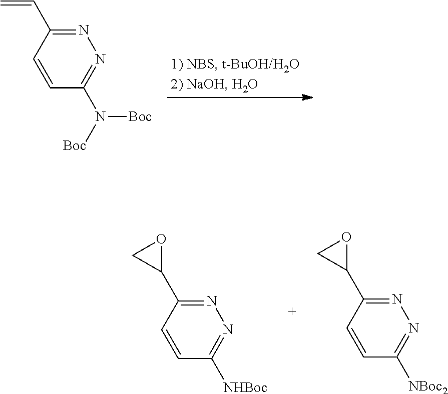

To a solution of tert-butyl (tert-butoxy carbonyl)(6-vinylpyridazin-3-yl)carbamate (12.0 g 37.3 mmol, 1.00 eq) int-BuOH (97.5 mL) and H₂O (195 mL) was added NBS (7.97 g, 44.8 mmol, 1.20 eq). The reaction mixture was stirred at 50° C. for 1 h, cooled to 0° C., and NaOH (4 M, 28.0 mL, 3.00 eq) was added dropwise. The reaction mixture was stirred at RT for 2 h. The reaction mixture was adjusted to pH 6 with 1 M HCl, then partitioned between EtOAc (200 mL) and H₂O (80.0 mL). The organic phase was separated, dried over Na₂SO₄, filtered, and concentrated under reduced pressure to give a residue. The residue was purified by column chromatography (SiO₂, PE:EtOAc=5:1 to 3:1) and further purified by prep-HPLC (neutral condition; Welch Ultimate XB—CN 250×70×10 μm; mobile phase: [Hexane-EtOH (0.1% NH₃H₂O)]; B %: 1%-40%, 15 min). The first eluting peak, tert-butyl (6-(oxiran-2-yl)pyridazin-3-yl)carbamate (1.10 g, 4.64 mmol, 12% yield), was obtained as a light-yellow solid. ¹H NMR (400 MHz, CDCl₃) δ 8.23 (d, J=9.2 Hz, 1H), 7.99 (s, 1H), 7.24-7.29 (m, 1H), 4.22-4.29 (m, 1H), 3.22-3.29 (m, 1H), 2.87-2.95 (m, 1H), 1.53 (s, 9H). The second eluting peak, tert-butyl (tert-butoxycarbonyl)(6-(oxiran-2-yl)pyridazin-3-yl)carbamate (2.40 g, 7.11 mmol, 19% yield), was obtained as a light-yellow solid. ¹H NMR (400 MHz, CDCl₃) δ 7.44-7.56 (m, 1H), 7.24-7.38 (m, 1H), 4.29-4.42 (m, 1H), 3.22-3.40 (s, 1H), 2.81-2.98 (m, 1H), 1.40-1.52 (m, 181H).

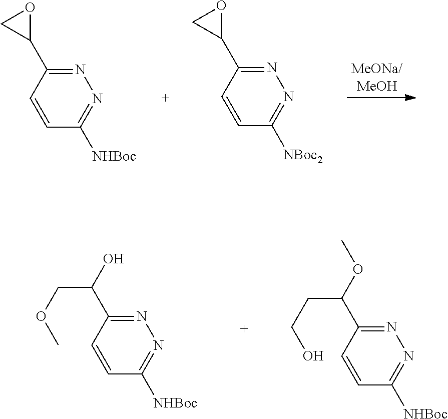

To a solution of tert-butyl (6-(oxiran-2-yl)pyridazin-3-yl)carbamate (1.00 g, 4.21 mmol, 1.00 eq) and tert-butyl (tert-butoxycarbonyl)(6-(oxiran-2-yl)pyridazin-3-yl)carbamate (2.40 g, 7.11 mmol, 1.69 eq) in MeOH (400 mL) was added CH₃ONa (454 mg, 8.42 mmol, 2.00 eq). The reaction mixture was stirred at 50° C. for 4 h. The reaction mixture was partitioned between EtOAc (100 mL) and H₂O (50.0 mL). The organic phase was separated, dried over Na₂SO₄, filtered, and concentrated under reduced pressure to give a residue. The residue was purified by column chromatography (SiO₂, PE/EtOAc=3/1 to 0/1). Secondary alcohol tert-butyl (6-(1-hydroxy-2-methoxyethyl)pyridazin-3-yl)carbamate (600 mg, 2.23 mmol, 53% yield) was obtained as a yellow oil. ¹H NMR (400 MHz, CDCl₃) δ 8.23-8.26 (m, 1H), 7.78 (s, 1H), 7.59-7.66 (m, 1H), 5.11-5.08 (m, 1H), 3.78-3.75 (m, 1H), 3.68-3.64 (m, 1H), 3.42-3.40 (m, 3H), 1.54 (s, 9H). Primary alcohol tert-butyl (6-(3-hydroxy-1-methoxypropyl)pyridazin-3-yl)carbamate (400 mg, 1.49 mmol, 35% yield) was obtained as a yellow oil. ¹H NMR (400 MHz, CDCl₃) δ 8.18-8.32 (m, 1H), 7.76 (s, 1H), 7.50-7.67 (m, 1H), 4.58-4.74 (m, 1H), 3.72-4.04 (m, 2H), 3.37-3.43 (m, 3H), 1.54 (s, 9H).

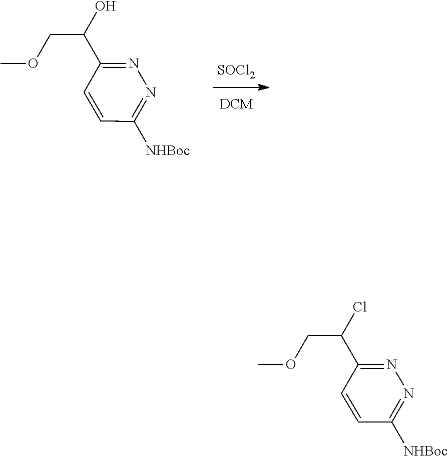

To a solution of secondary alcohol tert-butyl (6-(1-hydroxy-2-methoxyethyl)pyridazin-3-yl)carbamate (400 mg, 1.49 mmol, 1.00 eq) in DCM (5.00 mL) was added SOCl₂ (706 mg, 5.94 mmol, 431 μL, 4.00 eq). The reaction mixture was stirred at 50° C. for 4 h. The reaction was concentrated under reduced pressure to give a residue. The product, tert-butyl (6-(1-chloro-2-methoxyethyl)pyridazin-3-yl)carbamate (330 mg, 1.15 mmol, 77% yield), was obtained as a purple solid and used directly in the next step without further purification.

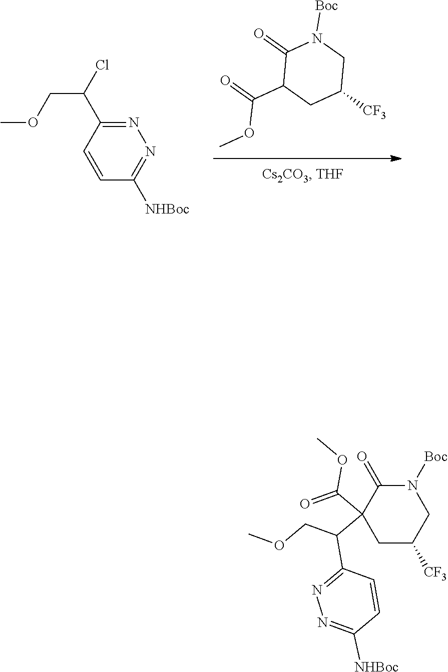 [SMILES: COCC(Cl)c1ccc(NC(=O)OC(C)(C)C)nn1 ; COC(=O)C1C[C@@H](C(F)(F)F)CN(C(=O)OC(C)(C)C)C1=O ; COCC(c1ccc(NC(=O)OC(C)(C)C)nn1)C1(C(=O)OC)C[C@@H](C(F)(F)F)CN(C(=O)OC(C)(C)C)C1=O]

To a solution of tert-butyl (6-(1-chloro-2-methoxyethyl)pyridazin-3-yl)carbamate (330 mg, 1.15 mmol, 1.00 eq) in THF (5.00 mL) was added Cs₂CO₃ (747 mg, 2.29 mmol, 2.00 eq) and 1-(tert-butyl)₃-methyl(5R)-2-oxo-5-(trifluoromethyl)piperidine-1,3-dicarboxylate (559 mg, 1.72 mmol, 1.50 eq). The reaction mixture was stirred at 70° C. for 2 h. The reaction mixture was partitioned between EtOAc (80.0 mL) and H₂O (60.0 mL). The organic phase was separated, dried over Na₂SO₄, filtered, and concentrated under reduced pressure to give a residue. The residue was purified by prep-TLC (SiO₂, PE:EtOAc=3:1). The product, 1-(tert-butyl) 3-methyl (5R)-3-(1-(6-((tert-butoxycarbonyl)amino)pyridazin-3-yl)-2-methoxyethyl)-2-oxo-5-(trifluoromethyl)piperidine-1,3-dicarboxylate (209 mg, 362 umol, 32% yield), was obtained as a yellow solid. LCMS [M+H]⁺=609.5 m/z.

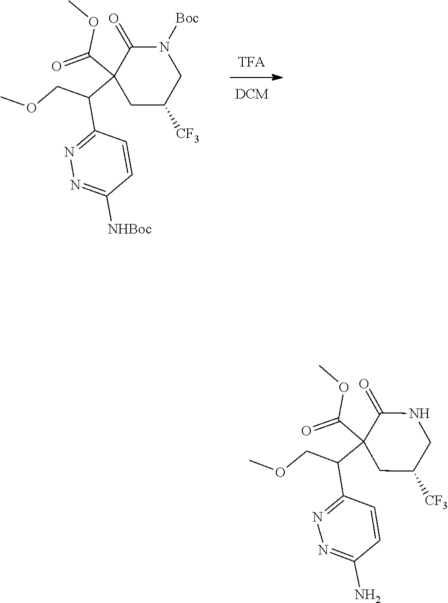 [SMILES: COCC(c1ccc(NC(=O)OC(C)(C)C)nn1)C1(C(=O)OC)C[C@@H](C(F)(F)F)CN(C(=O)OC(C)(C)C)C1=O ; COCC(c1ccc(N)nn1)C1(C(=O)OC)C[C@@H](C(F)(F)F)CNC1=O]

To a solution of 1-(tert-butyl) 3-methyl (5R)-3-(1-(6-((tert-butoxycarbonyl)amino)pyridazin-3-yl)-2-methoxyethyl)-2-oxo-5-(trifluoromethyl)piperidine-1,3-dicarboxylate (209 mg, 362 μmol, 1.00 eq) in DCM (1.00 mL) was added TFA (4.62 g, 40.5 mmol, 3.00 mL, 111 eq) at 0° C. The reaction mixture was stirred at RT for 1 h. The reaction was concentrated under reduced pressure to give a residue. The residue was purified by prep-HPLC (TFA condition; YMC Triart C18 150×25 mm×5 um; mobile phase: [water (TFA)-ACN]; B %: 20%-40%, 10 min). The product, methyl (5R)-3-(1-(6-aminopyridazin-3-yl)-2-methoxyethyl)-2-oxo-5-(trifluoromethyl)piperidine-3-carboxylate (170 mg, 346.69 μmol, 96% yield, TFA salt), was obtained as a yellow solid. LCMS [M+H]⁺=409.2 m/z.

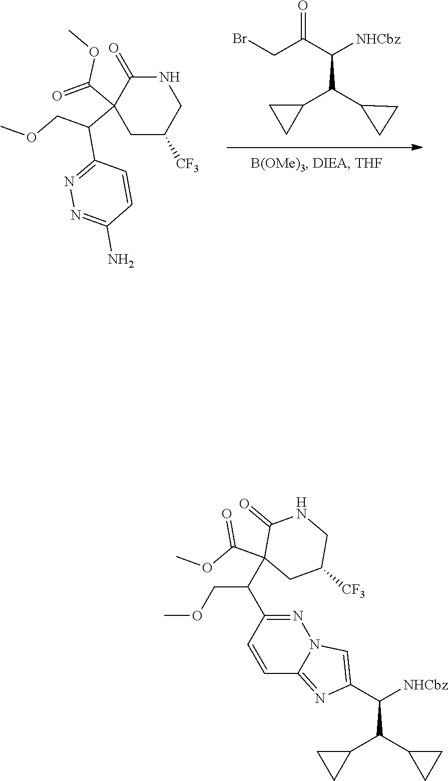

Methyl (5R)-3-(1-(2-((S)-1-(((benzyloxy)carbonyl)amino)-2,2-dicyclopropylethyl)imidazo[1,2-b]pyridazin-6-yl)-2-methoxyethyl)-2-oxo-5-(trifluoromethyl)piperidine-3-carboxylate was synthesized following General Procedure 1, using methyl(5R)-3-(1-(6-aminopyridazin-3-yl)-2-methoxyethyl)-2-oxo-5-(trifluoromethyl)piperidine-3-carboxylate (170 mg, 451 μmol, 1.00 eq), Intermediate 1 (257 mg, 677 μmol, 1.50 eq), B(OMe)₃ (920 mg, 8.85 mmol, 1.00 mL, 19.6 eq), and DIEA (2.23 g, 17.2 mmol, 3.00 mL, 38.1 eq). After 2 h at 70° C., crude product was purified by prep-TLC (SiO₂, EtOAc:MeOH=10:1). The product, methyl (5R)-3-(1-(2-((S)-1-(((benzyloxy)carbonyl)amino)-2,2-dicyclopropylethyl)imidazo[1,2-b]pyridazin-6-yl)-2-methoxyethyl)-2-oxo-5-(trifluoromethyl)piperidine-3-carboxylate (164 mg, 249 μmol, 55% yield), was obtained as a yellow solid. LCMS [M+H]⁺=658.5 m/z.

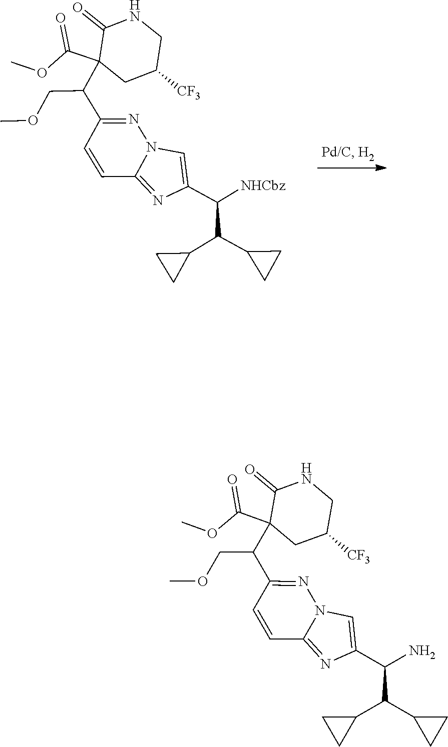

Methyl (5R)-3-(1-(2-((S)-1-amino-2,2-dicyclopropylethyl)imidazo[1,2-b]pyridazin-6-yl)-2-methoxyethyl)-2-oxo-5-(trifluoromethyl)piperidine-3-carboxylate was synthesized following General Procedure 3, using methyl (5R)-3-(1-(2-((S)-1-(((benzyloxy)carbonyl)amino)-2,2-dicyclopropylethyl)imidazo[1,2-b]pyridazin-6-yl)-2-methoxyethyl)-2-oxo-5-(trifluoromethyl)piperidine-3-carboxylate (164 mg, 249 μmol, 1.00 eq), Pd/C (20.0 mg) in DCM/MeOH, stirring under H₂ for 1 h. The product, methyl (5R)-3-(1-(2-((S)-1-amino-2,2-dicyclopropylethyl)imidazo[1,2-b]pyridazin-6-yl)-2-methoxyethyl)-2-oxo-5-(trifluoromethyl)piperidine-3-carboxylate (90.0 mg, 171 μmol, 69% yield), was obtained as a yellow solid. LCMS [M+H]⁺=524.2 m/z.

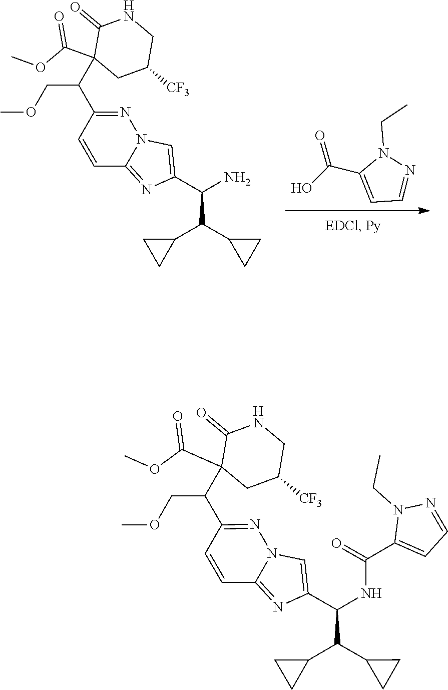

Methyl (5R)-3-(1-(2-((S)-2,2-dicyclopropyl-1-(1-ethyl-1H-pyrazole-5-carboxamido)ethyl)imidazo[1,2-b]pyridazin-6-yl)-2-methoxyethyl)-2-oxo-5-(trifluoromethyl)piperidine-3-carboxylate was synthesized following General Procedure 4, using methyl (5R)-3-(1-(2-((S)-1-amino-2,2-dicyclopropylethyl)imidazo[1,2-b]pyridazin-6-yl)-2-methoxyethyl)-2-oxo-5-(trifluoromethyl)piperidine-3-carboxylate (90.0 mg, 171 μmol, 1.00 eq), 1-ethyl-1H-pyrazole-5-carboxylic acid (36.1 mg, 257 μmol, 1.50 eq), EDCI (65.9 mg, 343 μmol, 2.00 eq), and HOBt (27.8 mg, 206 μmol, 1.20 eq), stirring for 12 h. The residue was purified by prep-TLC (SiO₂, EtOAc: MeOH=10:1). The product, methyl (5R)-3-(1-(2-((S)-2,2-dicyclopropyl-1-(1-ethyl-1H-pyrazole-5-carboxamido)ethyl)imidazo[1,2-b]pyridazin-6-yl)-2-methoxyethyl)-2-oxo-5-(trifluoromethyl)piperidine-3-carboxylate (70.0 mg, 108 μmol, 63% yield), was obtained as a white solid. LCMS [M+H]⁺=646.5 m/z.

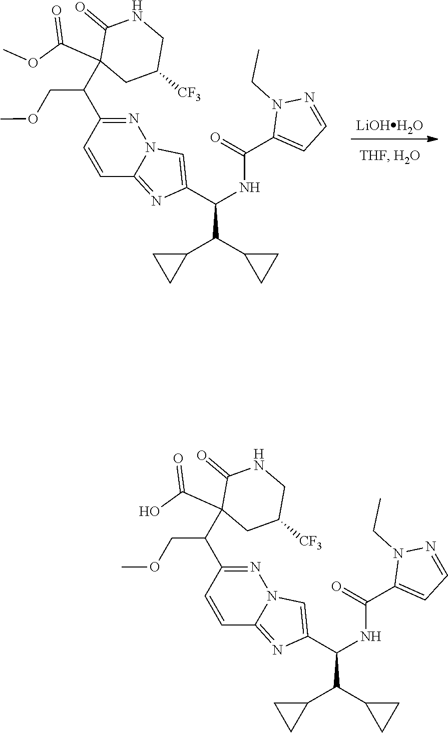

Intermediate (5R)-3-(1-(2-((S)-2,2-dicyclopropyl-l-(1-ethyl-1H-pyrazole-5-carboxamido)ethyl)imidazo[1,2-b]pyridazin-6-yl)-2-methoxyethyl)-2-oxo-5-(trifluoromethyl)piperidine-3-carboxylic acid was synthesized following General Procedure 5, using methyl (5R)-3-(1-(2-((S)-2,2-dicyclopropyl-1-(1-ethyl-1H-pyrazole-5-carboxamido)ethyl)imidazo[1,2-b]pyridazin-6-yl)-2-methoxyethyl)-2-oxo-5-(trifluoromethyl)piperidine-3-carboxylate (70.0 mg, 108 μmol, 1.00 eq), LiOH·H₂O (9.10 mg, 216.83 μmol, 2.00 eq), stirring for 2 h. The product, (5R)-3-(1-(2-((S)-2,2-dicyclopropyl-1-(1-ethyl-1H-pyrazole-5-carboxamido)ethyl)imidazo[1,2-b]pyridazin-6-yl)-2-methoxyethyl)-2-oxo-5-(trifluoromethyl)piperidine-3-carboxylic acid (60.0 mg, 94.9 μmol, 88% yield), was obtained as a white solid. LCMS [M+H]⁺=632.3 m/z.

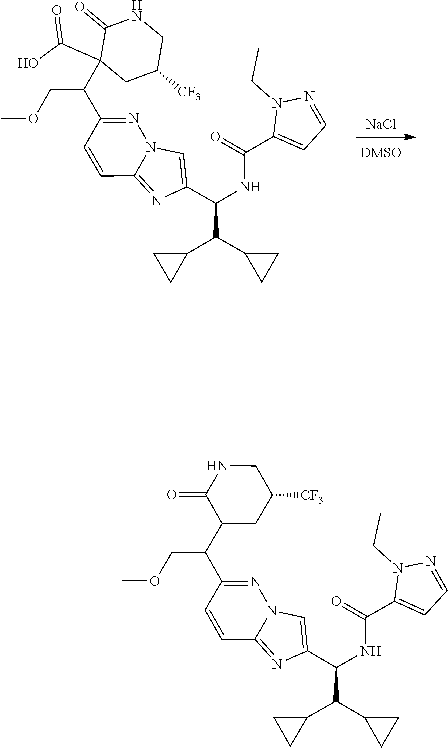

The mixture of stereoisomers, N-((1S)-2,2-dicyclopropyl-1-(6-(2-methoxy-1-((5R)-2-oxo-5-(trifluoromethyl)piperidin-3-yl)ethyl)imidazo[1,2-b]pyridazin-2-yl)ethyl)-1-ethyl-1H-pyrazole-5-carboxamide, was synthesized following General Procedure 6, using (5R)-3-(1-(2-((S)-2,2-dicyclopropyl-1-(1-ethyl-1H-pyrazole-5-carboxamido)ethyl)imidazo[1,2-b]pyridazin-6-yl)-2-methoxyethyl)-2-oxo-5-(trifluoromethyl)piperidine-3-carboxylic acid (60.0 mg, 94.9 μmol, 1.00 eq), DMSO (2.00 mL) and NaCl (27.7 mg, 474 μmol, 5.00 eq), stirring at 120° C. for 1 h. The residue was purified by prep-TLC (SiO₂, EtOAc: MeOH=10:1). The mixture of stereoisomers, N-((1S)-2,2-dicyclopropyl-1-(6-(2-methoxy-1-((5R)-2-oxo-5-(trifluoromethyl)piperidin-3-yl)ethyl)imidazo[1,2-b]pyridazin-2-yl)ethyl)-1-ethyl-1H-pyrazole-5-carboxamide (35.82 mg, crude), was obtained as a white solid. LCMS [M+H]⁺=588.4 m/z. The residue was purified by SFC (condition: DAICEL CHIRALPAK AS (250 mm×30 mm, 10 μm); mobile phase: [0.1% NH₃H₂O MeOH]; B %: 50%). The first eluting, single stereoisomer of N-((1S)-2,2-dicyclopropyl-1-(6-(2-methoxy-1-((5R)-2-oxo-5-(trifluoromethyl)piperidin-3-yl)ethyl)imidazo[1,2-b]pyridazin-2-yl)ethyl)-1-ethyl-1H-pyrazole-5-carboxamide (Compound 62, 8.45 mg, 13.3 μmol, 23% yield) was obtained as a yellow solid. LCMS [M+H]⁺=588.4 m/z.

### Example 64: Preparation of Compound 63

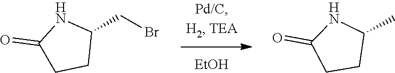

To a solution of (S)-5-(bromomethyl)pyrrolidin-2-one (4.50 g, 25.2 mmol, 1.00 eq) in EtOH (50.0 mL) was added Pd/C (500 mg, 25.2 mmol, 10% purity) and TEA (2.56 g, 25.2 mmol, 3.52 mL, 1.00 eq) under N₂. The suspension was degassed under vacuum and purged with H₂ several times. The reaction mixture was stirred under H₂ (15 psi) at RT for 16 h. The reaction mixture was filtered and to the filtrate was added MeOH (100 mL), then filtered again. The filtrate concentrated under reduced pressure to give a residue. The product, (R)-5-methylpyrrolidin-2-one (6.00 g, crude), was obtained as a white solid and used in the next step without further purification.

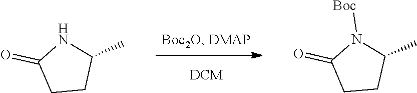

To a solution of (R)-5-methylpyrrolidin-2-one (6.00 g, 60.5 mmol, 1.00 eq) in DCM (60.0 mL) was added Boc₂O (19.8 g, 90.8 mmol, 20.9 mL, 1.50 eq) and DMAP (1.48 g, 12.1 mmol, 0.200 eq). The reaction mixture was stirred at RT for 12 h. The reaction mixture was concentrated under reduced pressure to remove DCM. The residue was purified by column chromatography (SiO₂, PE:EtOAc=1:0 to 0:1). The product, tert-butyl (R)-2-methyl-5-oxopyrrolidine-1-carboxylate (2.00 g, 10.0 mmol, 40% yield over 2 steps), was obtained as yellow oil. ¹H NMR (400 MHz, CDCl₃) δ 4.29-4.20 (m, 1H), 2.67-2.54 (m, 1H), 2.49-2.37 (m, 1H), 2.27-2.10 (m, 1H), 1.70-1.60 (m, 1H), 1.55-1.51 (m, 9H), 1.32 (d, J=6.4 Hz, 3H). LCMS [2M+Na]⁺=421.2 m/z.

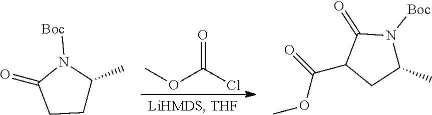

To a solution of tert-butyl (R)-2-methyl-5-oxopyrrolidine-1-carboxylate (2.00 g, 10.0 mmol, 1.00 eq) in THF (20.0 mL) was added LiHMDS (1 M, 20.0 mL, 2.00 eq) at −78° C. for 1 h. Then methyl chloroformate (2.37 g, 25.1 mmol, 1.94 mL, 2.50 eq) was added at −78° C. The reaction mixture was stirred at −78° C. for 1 h. The reaction mixture was diluted with saturated aqueous NH₄Cl (30.0 mL) and extracted with EtOAc 300 mL (100 mL×3), dried over Na₂SO₄, filtered and concentrated under reduced pressure to give a residue. The residue was purified by column chromatography (SiO₂, PE:EtOAc=1:0 to 0:1). The product, 1-(tert-butyl) 3-methyl (5R)-5-methyl-2-oxopyrrolidine-1,3-dicarboxylate (1.30 g, 5.05 mmol, 50% yield) was obtained as a white oil. ¹H NMR (400 MHz, CDCl₃) δ 4.38-4.09 (m, 1H), 3.82-3.78 (m, 3H), 3.72-3.48 (m, 1H), 2.66-2.38 (m, 1H), 2.13-1.87 (m, 1H), 1.57-1.51 (m, 9H), 1.46-1.30 (m, 3H). LCMS [2M+Na]⁺=537.3 m/z.

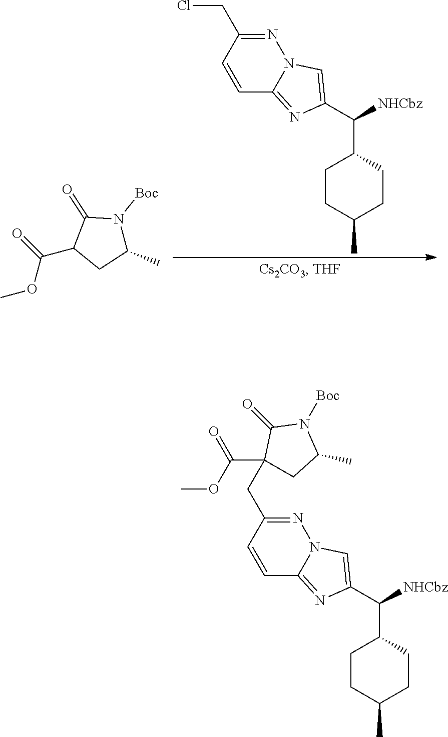

To a solution of 1-(tert-butyl) 3-methyl (5R)-5-methyl-2-oxopyrrolidine-1,3-dicarboxylate (300 mg, 1.17 mmol, 1.00 eq) and benzyl ((S)-(6-(chloromethyl)imidazo[1,2-b]pyridazin-2-yl)((1r,4S)-4-methylcyclohexyl)methyl)carbamate (547 mg, 1.28 mmol, 1.10 eq) in THE (10.0 mL) was added Cs₂CO₃ (760 mg, 2.33 mmol, 2.00 eq). The reaction mixture was stirred at 70° C. for 5 h. The residue was diluted with H₂O (100 mL) and extracted with EtOAc 300 mL (100 mL×3). The combined organic layers were dried over Na₂SO₄, filtered, and concentrated under reduced pressure to give a residue. The residue was purified by prep-TLC (SiO₂, DCM:MeOH=10:1, R_{f} (P1)=0.6). The product, 1-(tert-butyl) 3-methyl (5R)-3-((2-((S)-(((benzyloxy)carbonyl)amino)((1r,4S)-4-methylcyclohexyl)methyl)imidazo[1,2-b]pyridazin-6-yl)methyl)-5-methyl-2-oxopyrrolidine-1,3-dicarboxylate (350 mg, 540 μmol, 47% yield), was obtained as yellow oil. LCMS [M+H]⁺=648.4 m/z.

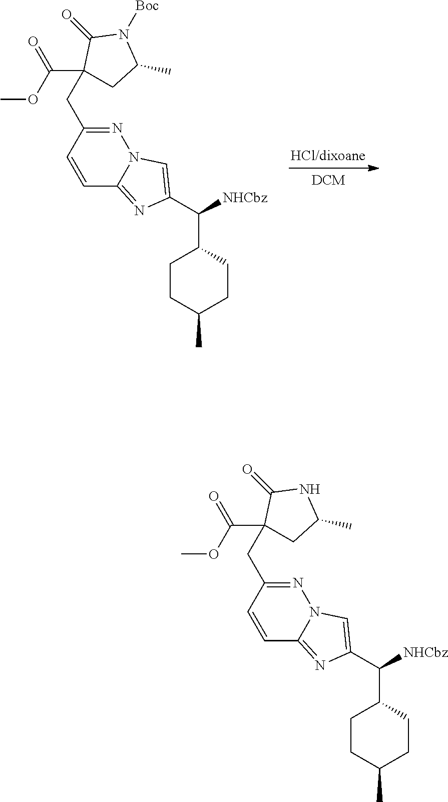 [SMILES: COC(=O)C1(Cc2ccc3nc([C@@H](NC(=O)OCc4ccccc4)[C@H]4CC[C@H](C)CC4)cn3n2)C[C@@H](C)N(C(=O)OC(C)(C)C)C1=O ; COC(=O)C1(Cc2ccc3nc([C@@H](NC(=O)OCc4ccccc4)[C@H]4CC[C@H](C)CC4)cn3n2)C[C@@H](C)NC1=O]

A solution of 1-(tert-butyl) 3-methyl (5R)-3-((2-((S)-(((benzyloxy)carbonyl)amino)((1r,4S)-4-methylcyclohexyl)methyl)imidazo[1,2-b]pyridazin-6-yl)methyl)-5-methyl-2-oxopyrrolidine-1,3-dicarboxylate (340 mg, 525 μmol, 1.00 eq) in DCM (10.0 mL) and HCl/dioxane (4 M, 3.00 mL, 22.9 eq) was stirred at RT for 2 h. The reaction mixture was concentrated under reduced pressure to remove HCl/dioxane and DCM. Methyl (5R)-3-((2-((S)-(((benzyloxy)carbonyl)amino)((1r,4S)-4-methylcyclohexyl)methyl)imidazo[1,2-b]pyridazin-6-yl)methyl)-5-methyl-2-oxopyrrolidine-3-carboxylate (280 mg, crude, HCl salt) was obtained as yellow oil. LCMS [M+H]⁺=548.4 m/z.

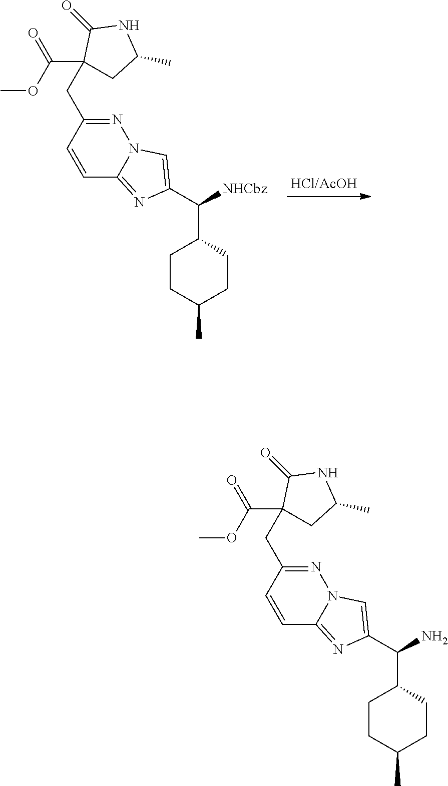 [SMILES: COC(=O)C1(Cc2ccc3nc([C@@H](NC(=O)OCc4ccccc4)[C@H]4CC[C@H](C)CC4)cn3n2)C[C@@H](C)NC1=O ; COC(=O)C1(Cc2ccc3nc([C@@H](N)[C@H]4CC[C@H](C)CC4)cn3n2)C[C@@H](C)NC1=O]

A solution of Methyl (5R)-3-((2-((S)-(((benzyloxy)carbonyl)amino)((1r,4S)-4-methylcyclohexyl)methyl)imidazo[1,2-b]pyridazin-6-yl)methyl)-5-methyl-2-oxopyrrolidine-3-carboxylate (270 mg, crude, HCl salt) in 12 M HCl (2.00 mL) and AcOH (210 mg, 3.50 mmol, 0.200 mL, 7.09 eq) was stirred at 60° C. for 30 min. The residue was adjusted with saturated aqueous NaHCO₃ to pH 7 and extracted with EtOAc (50 mL×3), dried over Na₂SO₄, filtered, and concentrated under reduced pressure to give a residue. Methyl (5R)-3-((2-((S)-amino((1r,4S)-4-methylcyclohexyl)methyl)imidazo[1,2-b]pyridazin-6-yl)methyl)-5-methyl-2-oxopyrrolidine-3-carboxylate (150 mg, crude, HCl salt) was obtained as a yellow solid. LCMS [M+H]⁺=414.3 m/z.

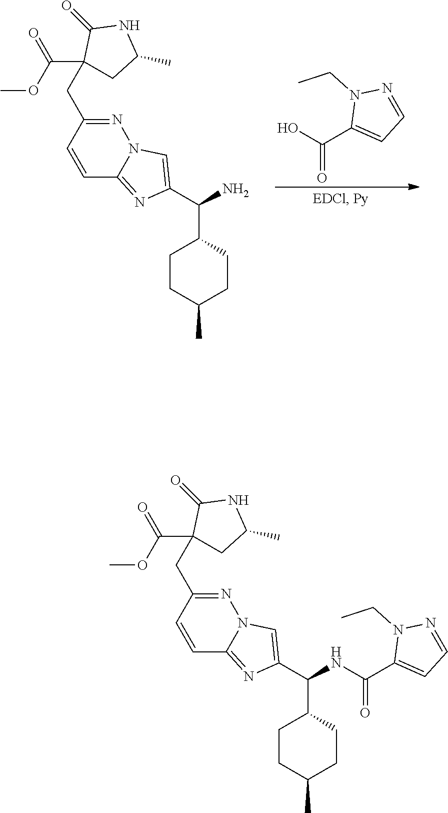

Methyl (5R)-3-((2-((S)-(1-ethyl-1H-pyrazole-5-carboxamido)((1r,4S)-4-methylcyclohexyl)methyl)imidazo[1,2-b]pyridazin-6-yl)methyl)-5-methyl-2-oxopyrrolidine-3-carboxylate was synthesized following General Procedure 4, using Methyl (5R)-3-((2-((S)-amino((1r,4S)-4-methylcyclohexyl)methyl)imidazo[1,2-b]pyridazin-6-yl)methyl)-5-methyl-2-oxopyrrolidine-3-carboxylate (140 mg, crude, HCl salt), 1-ethyl-1H-pyrazole-5-carboxylic acid (56.9 mg, 406 μmol, 1.20 eq), and EDCI (130 mg, 677 μmol, 2.00 eq), stirring for 2 h. The residue was purified by prep-TLC (SiO₂, DCM:MeOH=10:1, R_{f} (P1)=0.3). The product, methyl (5R)-3-((2-((S)-(1-ethyl-1H-pyrazole-5-carboxamido)((1r,4S)-4-methylcyclohexyl)methyl)imidazo[1,2-b]pyridazin-6-yl)methyl)-5-methyl-2-oxopyrrolidine-3-carboxylate (80.0 mg, 149 μmol, 32% yield over 3 steps), was obtained as a yellow solid. LCMS [M+H]⁺=536.3 m/z.

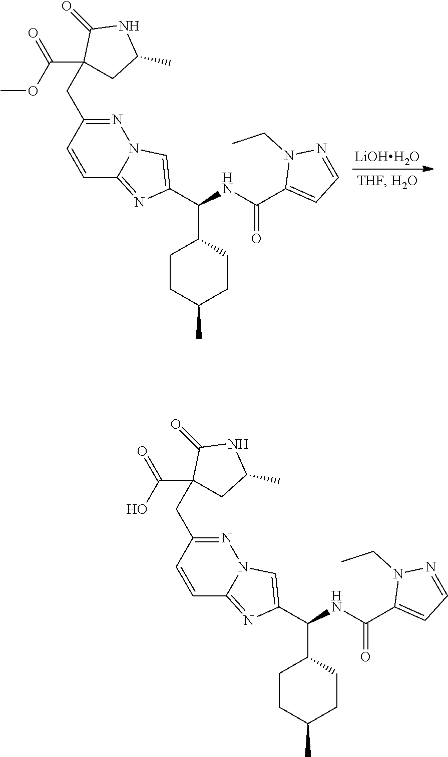

Intermediate (5R)-3-((2-((S)-(1-ethyl-1H-pyrazole-5-carboxamido)((1r,4S)-4-methylcyclohexyl)methyl)imidazo[1,2-b]pyridazin-6-yl)methyl)-5-methyl-2-oxopyrrolidine-3-carboxylic acid was synthesized following General Procedure 5, using methyl (5R)-3-((2-((S)-(1-ethyl-1H-pyrazole-5-carboxamido)((1r,4S)-4-methylcyclohexyl)methyl)imidazo[1,2-b]pyridazin-6-yl)methyl)-5-methyl-2-oxopyrrolidine-3-carboxylate (70.0 mg, 130 μmol, 1.00 eq), LiOH·H₂O (11.0 mg, 262 μmol, 2.00 eq), stirring for 1 h. The product, (5R)-3-((2-((S)-(1-ethyl-1H-pyrazole-5-carboxamido)((1r,4S)-4-methylcyclohexyl)methyl)imidazo[1,2-b]pyridazin-6-yl)methyl)-5-methyl-2-oxopyrrolidine-3-carboxylic acid (60.0 mg, 115.03 μmol, 88% yield), was obtained as a white solid. LCMS [M+H]⁺=522.2 m/z.

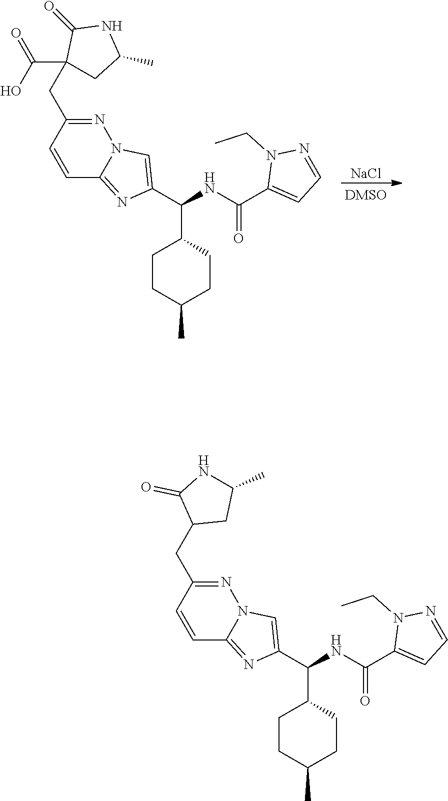

The mixture of stereoisomers, 1-ethyl-N-((1S)-(6-(((5R)-5-methyl-2-oxopyrrolidin-3-yl)methyl)imidazo[1,2-b]pyridazin-2-yl)((1r,4S)-4-methylcyclohexyl)methyl)-1H-pyrazole-5-carboxamide, was synthesized following General Procedure 6, using (5R)-3-((2-((S)-(1-ethyl-1H-pyrazole-5-carboxamido)((1r,4S)-4-methylcyclohexyl)methyl)imidazo[1,2-b]pyridazin-6-yl)methyl)-5-methyl-2-oxopyrrolidine-3-carboxylic acid (50.0 mg, 95.9 μmol, 1.00 eq), DMSO (1.00 mL) and NaCl (11.2 mg, 192 μmol, 2.00 eq), stirring at 120° C. for 1 h. The residue was purified by prep-TLC (SiO₂, DCM:MeOH=10:1). The mixture of stereoisomers, 1-ethyl-N-((1S)-(6-(((5R)-5-methyl-2-oxopyrrolidin-3-yl)methyl)imidazo[1,2-b]pyridazin-2-yl)((1r,4S)-4-methylcyclohexyl)methyl)-1H-pyrazole-5-carboxamide (40.0 mg, 83.75 μmol, 87% yield), was obtained as a white solid. LCMS [M+H]⁺=478.5 m/z. The residue was purified by prep-SFC (DAICEL CHIRALPAK AD (250 mm×30 mm, 10 μm); 35% mobile phase: [0.1% NH₃H₂O EtOH]). The second eluting, single stereoisomer of 1-ethyl-N-((1S)-(6-(((5R)-5-methyl-2-oxopyrrolidin-3-yl)methyl)imidazo[1,2-b]pyridazin-2-yl)((1r,4S)-4-methylcyclohexyl)methyl)-1H-pyrazole-5-carboxamide (Compound 63, 7.72 mg, 15.15 μmol, 18% yield), was obtained as a white solid. LCMS [M+H]⁺=478.2 m/z.

### Example 65: Preparation of Compound 64

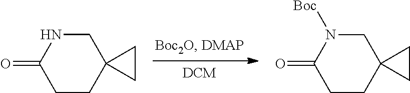 [SMILES: O=C1CCC2(CC2)CN1 ; CC(C)(C)OC(=O)N1CC2(CCC1=O)CC2]

To a solution of 5-azaspiro[2.5]octan-6-one (500 mg, 3.99 mmol, 1.00 eq) in DCM (5.00 mL) was added Boc₂O (1.74 g, 7.99 mmol, 1.84 mL, 2.00 eq) and DMAP (97.6 mg, 798 μmol, 0.200 eq). The reaction mixture was stirred at RT for 5 h. The reaction mixture was diluted with H₂O (40.0 mL) and extracted with DCM (40.0 mL×2). The combined organic layers were washed with brine (40.0 mL), dried over Na₂SO₄, filtered and concentrated under reduced pressure to give a residue. The residue was purified by column chromatography (SiO₂, PE:EtOAc=20:1 to 5:1). The product, tert-butyl 6-oxo-5-azaspiro[2.5]octane-5-carboxylate (700 mg, 3.11 mmol, 78% yield), was obtained as colorless oil. ¹H NMR (400 MHz, CDCl₃) δ 3.40 (s, 2H), 2.61 (t, J=6.8 Hz, 2H), 1.65 (t, J=7.2 Hz, 2H), 1.50 (s, 9H), 0.59-0.53 (m, 2H), 0.52-0.49 (m, 2H).

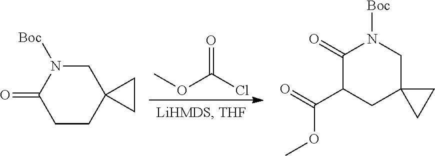

A solution of tert-butyl 6-oxo-5-azaspiro[2.5]octane-5-carboxylate (700 mg, 3.11 mmol, 1.00 eq) in THE (10.0 mL) was added dropwise LiHMDS (1 M, 6.21 mL, 2.00 eq) at −70° C., the reaction mixture was stirred at −70° C. for 1 h. Methyl chloroformate (734 mg, 7.77 mmol, 601 μL, 2.50 eq) was added dropwise at −70° C., the reaction mixture was stirred at −70° C. for 1 h. The reaction solution was quenched with (20.0 mL) saturated aqueous NH₄Cl solution. The residue was diluted with H₂O (40.0 mL) and extracted with EtOAc (40.0 mL×3), dried over Na₂SO₄, filtered, and concentrated under reduced pressure to give a residue. The product, 5-(tert-butyl) 7-methyl 6-oxo-5-azaspiro[2.5]octane-5,7-dicarboxylate (600 mg, 2.12 mmol, 68% yield), was obtained as yellow oil. LCMS [2M+Na]⁺=589.3 m/z.

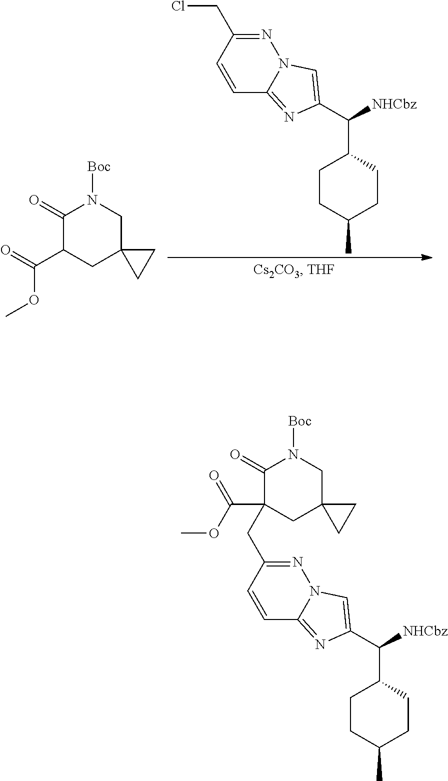 [SMILES: COC(=O)C1CC2(CC2)CN(C(=O)OC(C)(C)C)C1=O ; C[C@H]1CC[C@H]([C@H](NC(=O)OCc2ccccc2)c2cn3nc(CCl)ccc3n2)CC1 ; COC(=O)C1(Cc2ccc3nc([C@@H](NC(=O)OCc4ccccc4)[C@H]4CC[C@H](C)CC4)cn3n2)CC2(CC2)CN(C(=O)OC(C)(C)C)C1=O]

A mixture of 5-(tert-butyl) 7-methyl 6-oxo-5-azaspiro[2.5]octane-5,7-dicarboxylate (300 mg, 703 μmol, 1.00 eq), benzyl ((S)-(6-(chloromethyl)imidazo[1,2-b]pyridazin-2-yl)((1r,4S)-4-methylcyclohexyl)methyl)carbamate (219 mg, 773 μmol, 1.10 eq), Cs₂CO₃ (687 mg, 2.11 mmol, 3.00 eq) in THF (10.0 mL) was stirred at 70° C. for 2 h. The residue was diluted with H₂O 30.0 mL and extracted with EtOAc (30.0 mL×3). The combined organic layers were washed with brine (30.0 mL×2), dried over Na₂SO₄, filtered and concentrated under reduced pressure to give a residue. The residue was purified by prep-TLC (SiO₂, PE:EtOAc=1:1). The product, 5-(tert-butyl) 7-methyl 7-((2-((S)-(((benzyloxy)carbonyl)amino)((1r,4S)-4-methylcyclohexyl)methyl)imidazo[1,2-b]pyridazin-6-yl)methyl)-6-oxo-5-azaspiro[2.5]octane-5,7-dicarboxylate (300 mg, 445 μmol, 63% yield), was obtained as an off-white solid. LCMS [M+H]⁺=674.4 m/z.

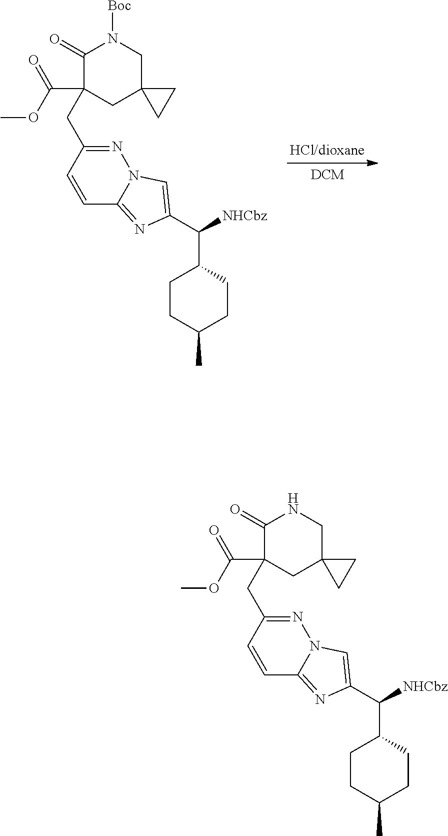 [SMILES: COC(=O)C1(Cc2ccc3nc([C@@H](NC(=O)OCc4ccccc4)[C@H]4CC[C@H](C)CC4)cn3n2)CC2(CC2)CN(C(=O)OC(C)(C)C)C1=O ; COC(=O)C1(Cc2ccc3nc([C@@H](NC(=O)OCc4ccccc4)[C@H]4CC[C@H](C)CC4)cn3n2)CC2(CC2)CNC1=O]

To a solution of 5-(tert-butyl) 7-methyl 7-((2-((S)-(((benzyloxy)carbonyl)amino)((1r,4S)-4-methylcyclohexyl)methyl)imidazo[1,2-b]pyridazin-6-yl)methyl)-6-oxo-5-azaspiro[2.5]octane-5,7-dicarboxylate (300 mg, 445 μmol, 1.00 eq) in DCM (5.00 mL) was added HCl/dioxane (4 M, 556 μL, 5.00 eq). The reaction mixture was stirred at RT for 2 h. The residue was concentrated under the vacuum to give a residue. The residue was used in the next step without any purification. Methyl 7-((2-((S)-(((benzyloxy)carbonyl)amino)((1r,4S)-4-methylcyclohexyl)methyl)imidazo[1,2-b]pyridazin-6-yl)methyl)-6-oxo-5-azaspiro[2.5]octane-7-carboxylate (220 mg, 360.57 μmol, 81% yield, HCl salt) was obtained as a yellow oil. LCMS [M+H]⁺=574.4 m/z.

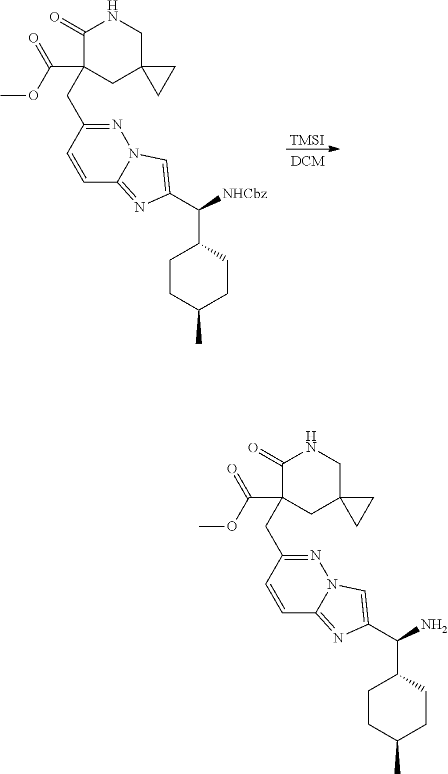 [SMILES: "TMSI" ; COC(=O)C1(Cc2ccc3nc([C@@H](NC(=O)OCc4ccccc4)[C@H]4CC[C@H](C)CC4)cn3n2)CC2(CC2)CNC1=O ; COC(=O)C1(Cc2ccc3nc([C@@H](N)[C@H]4CC[C@H](C)CC4)cn3n2)CC2(CC2)CNC1=O]

Methyl 7-((2-((S)-amino((1r,4S)-4-methylcyclohexyl)methyl)imidazo[1,2-b]pyridazin-6-yl)methyl)-6-oxo-5-azaspiro[2.5]octane-7-carboxylate was synthesized following General Procedure 2, using methyl 7-((2-((S)-(((benzyloxy)carbonyl)amino)((1r,4S)-4-methylcyclohexyl)methyl)imidazo[1,2-b]pyridazin-6-yl)methyl)-6-oxo-5-azaspiro[2.5]octane-7-carboxylate (200 mg, 348 μmol, 1.00 eq) and TMSI (140 mg, 700 μmol, 95.0 μL, 2.00 eq). After 1 h at 0° C., the product, methyl 7-((2-((S)-amino((1r,4S)-4-methylcyclohexyl)methyl)imidazo[1,2-b]pyridazin-6-yl)methyl)-6-oxo-5-azaspiro[2.5]octane-7-carboxylate (120 mg, 273 μmol, 78% yield), was obtained as a yellow oil. LCMS [M+H]⁺=440.3 m/Z.

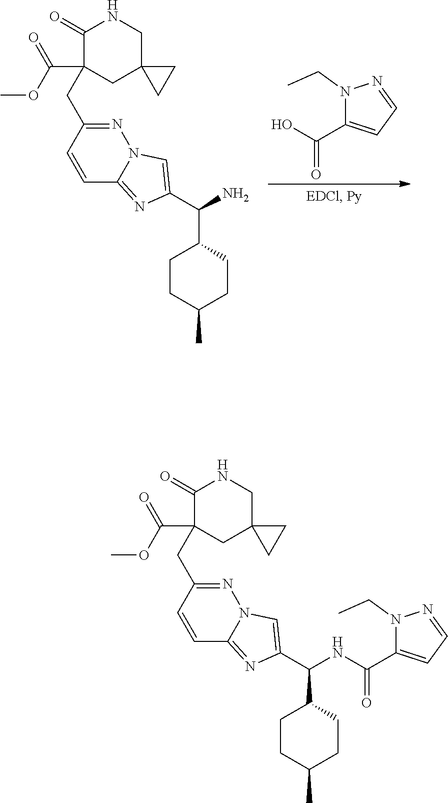

Methyl 7-((2-((S)-(1-ethyl-1H-pyrazole-5-carboxamido)((1r,4S)-4-methylcyclohexyl)methyl)imidazo[1,2-b]pyridazin-6-yl)methyl)-6-oxo-5-azaspiro[2.5]octane-7-carboxylate was synthesized following General Procedure 4, using methyl 7-((2-((S)-amino((1r,4S)-4-methylcyclohexyl)methyl)imidazo[1,2-b]pyridazin-6-yl)methyl)-6-oxo-5-azaspiro[2.5]octane-7-carboxylate (110 mg, 250 μmol, 1.00 eq), 1-ethyl-1H-pyrazole-5-carboxylic acid (38.5 mg, 275 μmol, 1.10 eq), and EDCI (144 mg, 751 μmol, 3.00 eq), stirring for 1 h. The product, methyl 7-((2-((S)-(1-ethyl-1H-pyrazole-5-carboxamido)((1r,4S)-4-methylcyclohexyl)methyl)imidazo[1,2-b]pyridazin-6-yl)methyl)-6-oxo-5-azaspiro[2.5]octane-7-carboxylate (120 mg, 213 μmol, 85% yield), was obtained as a yellow oil and used in the next step without purification. LCMS [M+H]⁺=562.4 m/z.

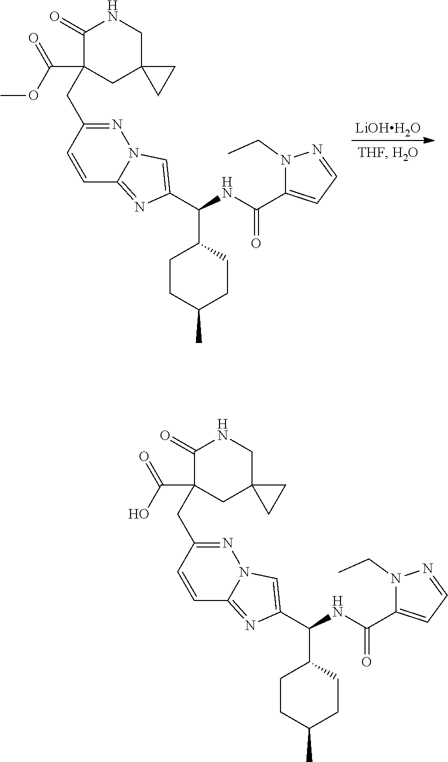

Intermediate 7-((2-((S)-(1-ethyl-1H-pyrazole-5-carboxamido)((1r,4S)-4-methylcyclohexyl)methyl)imidazo[1,2-b]pyridazin-6-yl)methyl)-6-oxo-5-azaspiro[2.5]octane-7-carboxylic acid was synthesized following General Procedure 5, using methyl 7-((2-((S)-(1-ethyl-1H-pyrazole-5-carboxamido)((1r,4S)-4-methylcyclohexyl)methyl)imidazo[1,2-b]pyridazin-6-yl)methyl)-6-oxo-5-azaspiro[2.5]octane-7-carboxylate (120 mg, 213. μmol, 1.00 eq), LiOH·H₂O (44.8 mg, 1.07 mmol, 5.00 eq), stirring for 2 h. The product, 7-((2-((S)-(1-ethyl-1H-pyrazole-5-carboxamido)((1r,4S)-4-methylcyclohexyl)methyl)imidazo[1,2-b]pyridazin-6-yl)methyl)-6-oxo-5-azaspiro[2.5]octane-7-carboxylic acid (100 mg, 123 μmol, 58% yield, 67% purity), was obtained as a yellow solid. LCMS [M+H]⁺=548.3 m/z.

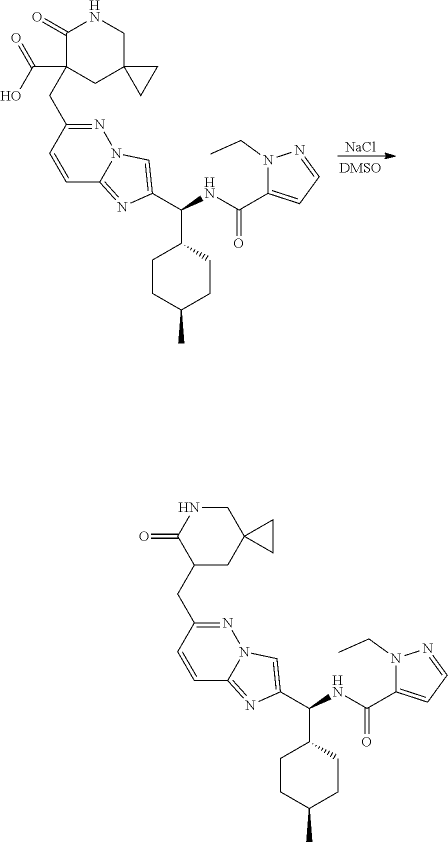

The mixture of stereoisomers, 1-ethyl-N-((1S)-((1r,4S)-4-methylcyclohexyl)(6-((6-oxo-5-azaspiro[2.5]octan-7-yl)methyl)imidazo[1,2-b]pyridazin-2-yl)methyl)-1H-pyrazole-5-carboxamide, was synthesized following General Procedure 6, using 7-((2-((S)-(1-ethyl-1H-pyrazole-5-carboxamido)((1r,4S)-4-methylcyclohexyl)methyl)imidazo[1,2-b]pyridazin-6-yl)methyl)-6-oxo-5-azaspiro[2.5]octane-7-carboxylic acid (90.0 mg, 164 μmol, 1.00 eq), DMSO (1.50 mL) and NaCl (48.0 mg, 822 μmol, 5.00 eq), stirring at 120° C. for 1 h. The residue was purified by prep-TLC (SiO₂, DCM:MeOH=10:1, R_{f}=0.35) and further purified by prep-SFC. The mixture of stereoisomers, 1-ethyl-N-((1S)-((1r,4S)-4-methylcyclohexyl)(6-((6-oxo-5-azaspiro[2.5]octan-7-yl)methyl)imidazo[1,2-b]pyridazin-2-yl)methyl)-1H-pyrazole-5-carboxamide (40.0 mg, 75.0 μmol, 46% yield), was obtained as a white solid. LCMS [M+H]⁺=504.4 m/z. The mixture of stereoisomers was purified by prep-SFC (DAICEL CHIRALCEL OD (250 mm×30 mm, 10 μm); 40% mobile phase: [0.1% NH₃·H₂O MeOH]). The second eluting single stereoisomer of 1-ethyl-N-((1S)-((1r,4S)-4-methylcyclohexyl)(6-((6-oxo-5-azaspiro[2.5]octan-7-yl)methyl)imidazo[1,2-b]pyridazin-2-yl)methyl)-1H-pyrazole-5-carboxamide (Compound 64, 10.36 mg, 19.7 μmol, 24% yield) was obtained as a white solid. LCMS [M+H]⁺=504.3 m/z.

### Example 66: Preparation of Compounds 65 and 66

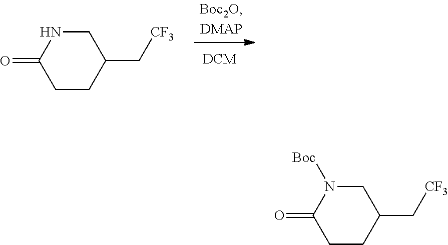 [SMILES: O=C1CCC(CC(F)(F)F)CN1 ; CC(C)(C)OC(=O)N1CC(CC(F)(F)F)CCC1=O]

To a solution of 5-(2,2,2-trifluoroethyl)piperidin-2-one (900 mg, 4.97 mmol, 1.00 eq) in DCM (10.0 mL) was added DMAP (60.6 mg, 496 μmol, 0.10 eq) and Boc₂O (1.63 g, 7.45 mmol, 1.71 mL, 1.50 eq). The reaction mixture was stirred at RT for 12 h. The reaction mixture was partitioned between DCM (60.0 mL) and H₂O (60.0 mL). The organic phase was separated, dried over Na₂SO₄, filtered, and concentrated under reduced pressure to give a residue. The residue was purified by column chromatography (SiO₂, PE:EtOAc=10:1 to 3:1). The product, tert-butyl 2-oxo-5-(2,2,2-trifluoroethyl)piperidine-1-carboxylate (1.12 g, 3.98 mmol, 80% yield), was obtained as a colorless oil. ¹H NMR (400 MHz, CDCl₃) δ 3.89-3.93 (m, 1H), 3.29-3.35 (m, 1H), 2.44-2.70 (m, 2H), 2.03-2.34 (m, 4H), 1.57-1.65 (m, 1H), 1.52 (s, 9H).

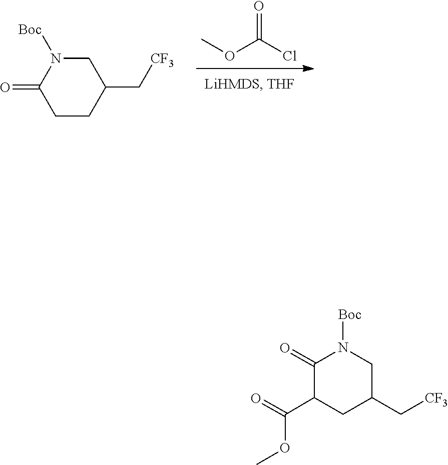

To a solution of tert-butyl 2-oxo-5-(2,2,2-trifluoroethyl)piperidine-1-carboxylate (1.12 g 3.91 mmol, 1.00 eq) in THF (11.0 mL) was added LiHMDS (1 M, 8.21 mL, 2.10 eq) at −70° C. for 1 h under N₂ atmosphere, then added methyl chloroformate (628 mg, 6.65 mmol, 513 μL, 1.70 eq) at −70° C. The reaction mixture was stirred at −70° C. for 1 h. The reaction mixture was quenched by addition of saturated aqueous NH₄Cl (40.0 mL) at 0° C., and then diluted with H₂O (80.0 mL) and extracted with EtOAc (80.0 mL). The organic phase was separated, dried over Na₂SO₄, filtered, and concentrated under reduced pressure to give a residue. The residue was purified by column chromatography (SiO₂, PE:EtOAc=10:1 to 3:1). The product, 1-(tert-butyl) 3-methyl 2-oxo-5-(2,2,2-trifluoroethyl)piperidine-1,3-dicarboxylate (1.15 g, 3.39 mmol, 87% yield), was obtained as a colorless oil. ¹H NMR (400 MHz, CDCl₃) δ 3.89-4.03 (m, 1H), 3.79 (s, 3H), 3.54-3.63 (m, 1H), 3.32-3.41 (m, 1H), 2.13-2.55 (m, 4H), 1.95-2.07 (m, 1H), 1.49-1.56 (m, 9H).

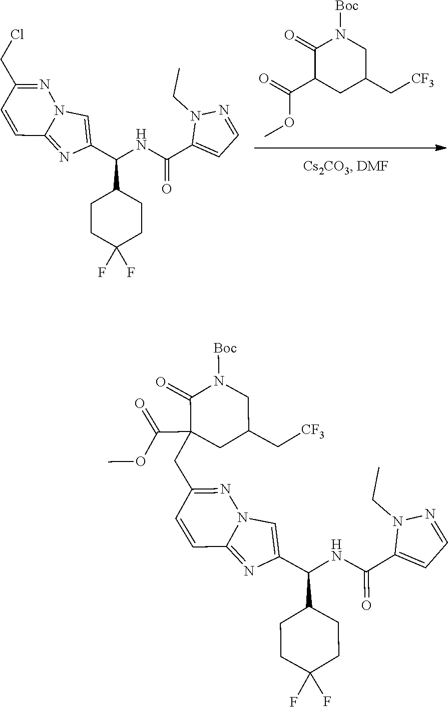

To a solution of 1-(tert-butyl) 3-methyl 2-oxo-5-(2,2,2-trifluoroethyl)piperidine-1,3-dicarboxylate (349 mg, 1.03 mmol, 1.50 eq) in DMF (8.00 mL) was added Cs₂CO₃ (894 mg, 2.75 mmol, 4.00 eq) and (S)—N-((6-(chloromethyl)imidazo[1,2-b]pyridazin-2-yl)(4,4-difluorocyclohexyl)methyl)-1-ethyl-1H-pyrazole-5-carboxamide (300 mg, 686.68 μmol, 1.00 eq). The reaction mixture was stirred at RT for 2 h. The reaction mixture was partitioned between EtOAc (60.0 mL) and H₂O (60.0 mL). The organic phase was separated, dried over Na₂SO₄, filtered, and concentrated under reduced pressure to give a residue. The residue was purified by prep-HPLC (neutral condition; Waters Xbridge C18 150×50 mm×10 μm; mobile phase: [water (NH₄HCO₃)-ACN]; B %: 43%-73%, 10 min). The product, 1-(tert-butyl)₃-methyl 3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(2,2,2-trifluoroethyl)piperidine-1,3-dicarboxylate (320 mg, 432 μmol, 63% yield) was obtained as a white solid. LCMS [M+H]⁺=740.3 m/z.

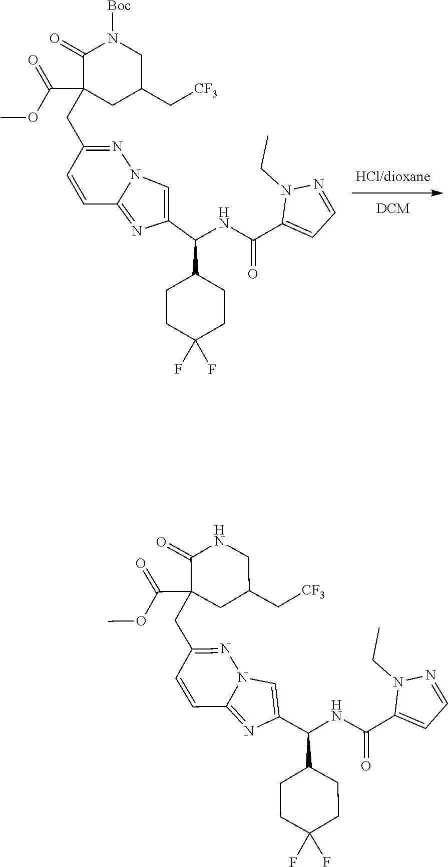

To a solution of 1-(tert-butyl) 3-methyl 3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(2,2,2-trifluoroethyl)piperidine-1,3-dicarboxylate (300 mg, 405 μmol, 1.00 eq) in DCM (1.50 mL) was added HCl/dioxane (4 M, 1.50 mL). The reaction mixture was stirred at RT for 1 h. The reaction was concentrated under reduced pressure to give a residue. The product, methyl 3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(2,2,2-trifluoroethyl)piperidine-3-carboxylate (260 mg, 384 μmol, 95% yield, HCl salt), was obtained as a yellow solid. LCMS [M+H]⁺=640.4 m/z.

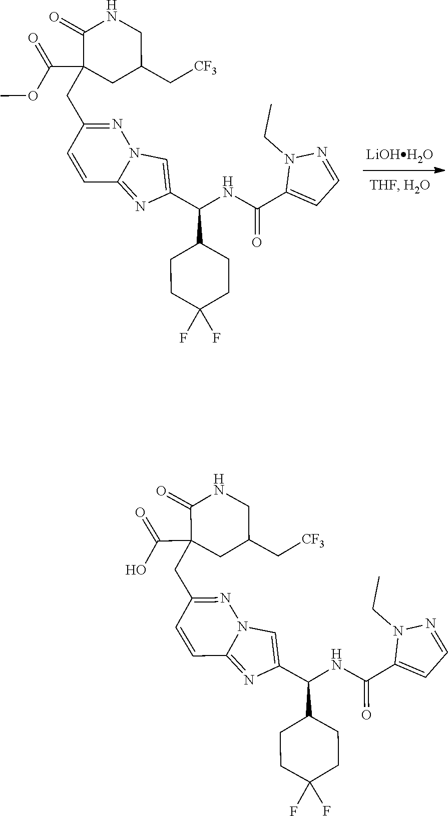

Intermediate 3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(2,2,2-trifluoroethyl)piperidine-3-carboxylic acid was synthesized following General Procedure 5, using methyl 3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(2,2,2-trifluoroethyl)piperidine-3-carboxylate (260 mg, 384 μmol, 1.00 eq, HCl salt), LiOH·H₂O (32.2 mg, 769 μmol, 2.00 eq), stirring for 1 h. The product, 3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(2,2,2-trifluoroethyl)piperidine-3-carboxylic acid (230 mg, 367 μmol, 96% yield), was obtained as a white solid. LCMS [M+H]⁺=626.4 m/z.

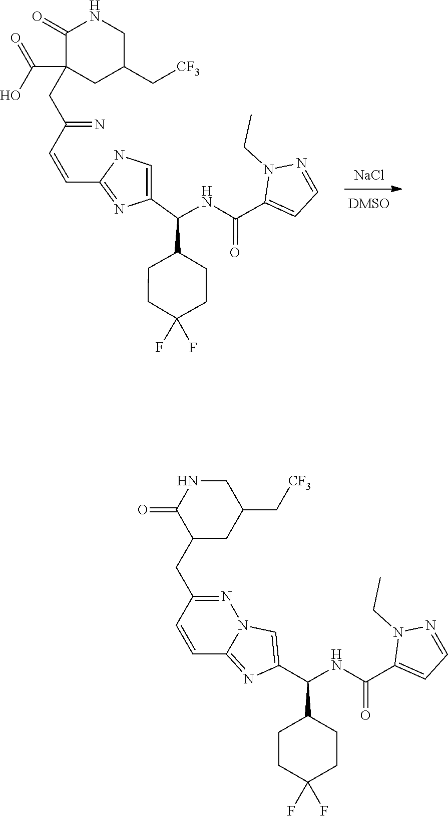

The mixture of stereoisomers, N-((1S)-(4,4-difluorocyclohexyl)(6-((2-oxo-5-(2,2,2-trifluoroethyl)piperidin-3-yl)methyl)imidazo[1,2-b]pyridazin-2-yl)methyl)-1-ethyl-1H-pyrazole-5-carboxamide was synthesized following General Procedure 6, using 3-((2-((S)-(4,4-difluorocyclohexyl)(1-ethyl-1H-pyrazole-5-carboxamido)methyl)imidazo[1,2-b]pyridazin-6-yl)methyl)-2-oxo-5-(2,2,2-trifluoroethyl)piperidine-3-carboxylic acid (230 mg, 367 μmol, 1.00 eq), DMSO (2.00 mL) and NaCl (42.9 mg, 735 μmol, 2.00 eq), stirring at 120° C. for 1 h. The residue was purified by prep-TLC (SiO₂, DCM:MeOH=10:1). The mixture of stereoisomers, N-((1S)-(4,4-difluorocyclohexyl)(6-((2-oxo-5-(2,2,2-trifluoroethyl)piperidin-3-yl)methyl)imidazo[1,2-b]pyridazin-2-yl)methyl)-1-ethyl-1H-pyrazole-5-carboxamide (200 mg, 343 μmol, 94% yield), was obtained as a white solid. LCMS [M+H]⁺=582.4 m/z. The mixture of stereoisomers, N-((1S)-(4,4-difluorocyclohexyl)(6-((2-oxo-5-(2,2,2-trifluoroethyl)piperidin-3-yl)methyl)imidazo[1,2-b]pyridazin-2-yl)methyl)-1-ethyl-1H-pyrazole-5-carboxamide, was purified by SFC (condition: DAICEL CHIRALPAK IC (250 mm×30 mm, 10 um); 35% mobile phase: [0.1% NH₃H₂O EtOH]), then further purified by SFC (condition: REGIS (S,S) WHELK-O1 (250 mm×25 mm, 10 um); 40% mobile phase: [0.1% NH₃H₂O IPA]). The first eluting, single stereoisomer of N-((1S)-(4,4-difluorocyclohexyl)(6-((2-oxo-5-(2,2,2-trifluoroethyl)piperidin-3-yl)methyl)imidazo[1,2-b]pyridazin-2-yl)methyl)-1-ethyl-1H-pyrazole-5-carboxamide (Compound 65, 41.86 mg, 70.9 μmol, 21% yield) was obtained as a white solid. LCMS [M+H]+=582.4m z. The third eluting, single stereoisomer of N-((1S)-(4,4-difluorocyclohexyl)(6-((2-oxo-5-(2,2,2-trifluoroethyl)piperidin-3-yl)methyl)imidazo[1,2-b]pyridazin-2-yl)methyl)-1-ethyl-1H-pyrazole-5-carboxamide (Compound 66, 36.26 mg, 61.9 μmol, 18% yield) was obtained as a white solid. LCMS [M+H]⁺=582.4 m/z.

### Example 67: Preparation of Compound 67

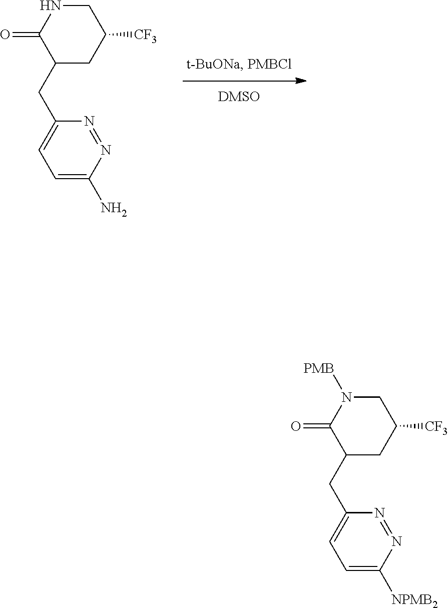

To a solution of (5R)-3-((6-aminopyridazin-3-yl)methyl)-5-(trifluoromethyl)piperidin-2-one (3.00 g, 10.9 mmol, 1.00 eq) and PMB-Cl (6.00 g, 38.3 mmol, 5.20 mL, 3.50 eq) in DMSO (5.00 mL) was added t-BuONa (3.68 g, 38.2 mmol, 3.50 eq). The reaction mixture was stirred at RT for 4 h. combined with a second crude reaction mixture for work up. The combined mixture was diluted with H₂O (40.0 mL) and extracted with EtOAc (40.0 mL×3). The combined organic layers were dried over Na₂SO₄, filtered, and concentrated under reduced pressure to give a residue. The crude product was purified by column chromatography (SiO₂, PE/EtOAc=0/1 to I/O). The product, (5R)-3-((6-(bis(4-methoxybenzyl)amino)pyridazin-3-yl)methyl)-1-(4-methoxybenzyl)-5-(trifluoromethyl)piperidin-2-one (1.32 g, 2.08 mmol) was obtained as yellow oil. LCMS [M+H]⁺=635.4 m/z.

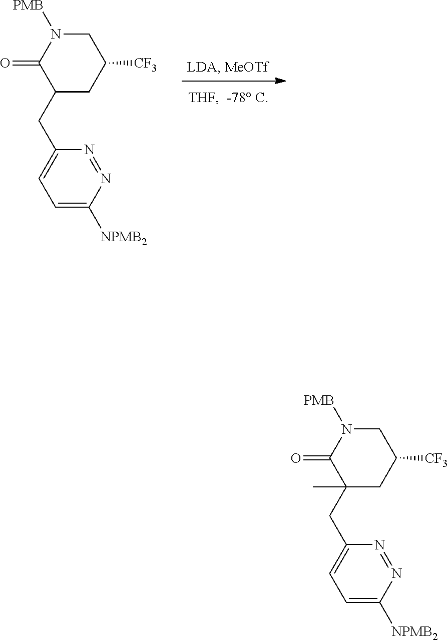

To a solution of (5R)-3-((6-(bis(4-methoxybenzyl)amino)pyridazin-3-yl)methyl)-1-(4-methoxybenzyl)-5-(trifluoromethyl)piperidin-2-one (1.30 g, 2.05 mmol, 1.00 eq) in THF (10.0 mL) was added LDA (2 M, 2.05 mL, 2.00 eq) at −78° C. for 30 min, then methyl trifluoromethanesulfonate (840 mg, 5.12 mmol, 561 μL, 2.50 eq) was added at −78° C. The reaction mixture was stirred at −78° C. for 1.5 h. The reaction mixture was then diluted with saturated aqueous NH₄Cl (40.0 mL) and extracted with EtOAc (40.0 mL×3), dried over Na₂SO₄, filtered, and concentrated under reduced pressure to give a residue. The residue was purified by column chromatography (SiO₂, PE/EtOAc=0/1 to 1/0). The product, (5R)-3-((6-(bis(4-methoxybenzyl)amino)pyridazin-3-yl)methyl)-1-(4-methoxybenzyl)-3-methyl-5-(trifluoromethyl)piperidin-2-one (700 mg, 1.08 mmol, 53% yield), was obtained as yellow oil. LCMS [M+H]⁺=649.4 m/z.

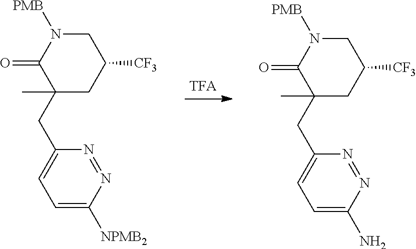

A solution of (5R)-3-((6-(bis(4-methoxybenzyl)amino)pyridazin-3-yl)methyl)-1-(4-methoxybenzyl)-3-methyl-5-(trifluoromethyl)piperidin-2-one (700 mg, 1.08 mmol, 1.00 eq) in TFA (3.00 mL) was stirred at 60° C. for 1 h. The reaction mixture was concentrated under reduced pressure to remove TFA. The crude residue was diluted with H₂O (40.0 mL) and washed with EtOAc (40.0 mL×3). The aqueous phase was adjusted with saturated aqueous NaHCO₃ to pH 8 and extracted with EtOAc (30.0 mL×3). The combined organic layers were dried over Na₂SO₄, filtered, and concentrated under reduced pressure to give a residue. The residue was purified by prep-HPLC (FA condition; Phenomenex Luna C18 150×25 mm×10 μm; mobile phase: [water (FA)-ACN]; B %: 0%, isocratic elution mode) to give desired compound, which was further separated by prep-HPLC (HCl condition; YMC Triart C18 150×25 mm×5 um; mobile phase: [water (HCl)-ACN]; gradient: 23%-53% B over 8 min). The product, (5R)-3-((6-aminopyridazin-3-yl)methyl)-1-(4-methoxybenzyl)-3-methyl-5-(trifluoromethyl)piperidin-2-one (160 mg, 391 μmol, 36% yield) was obtained as yellow oil. LCMS [M+H]⁺=409.2 m/z.

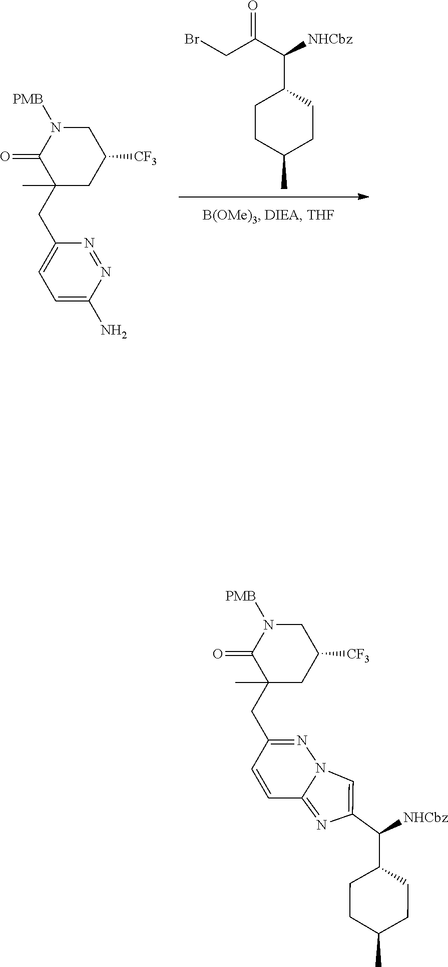

Benzyl ((1S)-(6-(((5R)-1-(4-methoxybenzyl)-3-methyl-2-oxo-5-(trifluoromethyl)piperidin-3-yl)methyl)imidazo[1,2-b]pyridazin-2-yl)((1r,4S)-4-methylcyclohexyl)methyl)carbamate was synthesized following General Procedure 1, using (5R)-3-((6-aminopyridazin-3-yl)methyl)-1-(4-methoxybenzyl)-3-methyl-5-(trifluoromethyl)piperidin-2-one (150 mg, 367 μmol, 1.00 eq), Intermediate 4 (168 mg, 440 μmol, 1.20 eq), B(OMe)₃ (190 mg, 1.84 mmol, 207 μL, 5.00 eq), and DIEA (237 mg, 1.84 mmol, 319 μL, 5.00 eq). After 2 h at 70° C., crude product was purified by prep-TLC (SiO₂, PE:EA=1:1, R_{f} (P1)=0.22). The product, benzyl ((1S)-(6-(((5R)-1-(4-methoxybenzyl)-3-methyl-2-oxo-5-(trifluoromethyl)piperidin-3-yl)methyl)imidazo[1,2-b]pyridazin-2-yl)((1r,4S)-4-methylcyclohexyl)methyl)carbamate (160 mg, 231 μmol, 63% yield), was obtained as a yellow oil. LCMS [M+H]⁺=692.5 m/z.

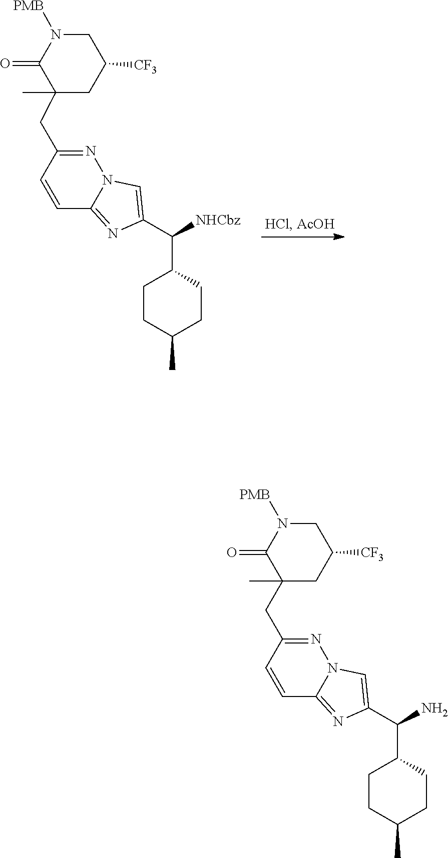

A solution of benzyl ((1S)-(6-(((5R)-1-(4-methoxybenzyl)-3-methyl-2-oxo-5-(trifluoromethyl)piperidin-3-yl)methyl)imidazo[1,2-b]pyridazin-2-yl)((1r,4S)-4-methylcyclohexyl)methyl)carbamate (140 mg, 202 μmol, 1.00 eq) in 12 M HCl (2.00 mL) and AcOH (0.200 mL) was stirred at 60° C. for 1 h. The reaction mixture was diluted with saturated aqueous NaHCO₃ (30.0 mL) and extracted with EtOAc (30.0 mL×3). The combined organic layers were dried over Na₂SO₄, filtered, and concentrated under reduced pressure to give a residue. The product, (5R)-3-((2-((S)-amino((1r,4S)-4-methylcyclohexyl)methyl)imidazo[1,2-b]pyridazin-6-yl)methyl)-1-(4-methoxybenzyl)-3-methyl-5-(trifluoromethyl)piperidin-2-one (100 mg, 179 μmol, 89% yield), was obtained as a yellow solid. LCMS [M+H]⁺=558.7 m/z.

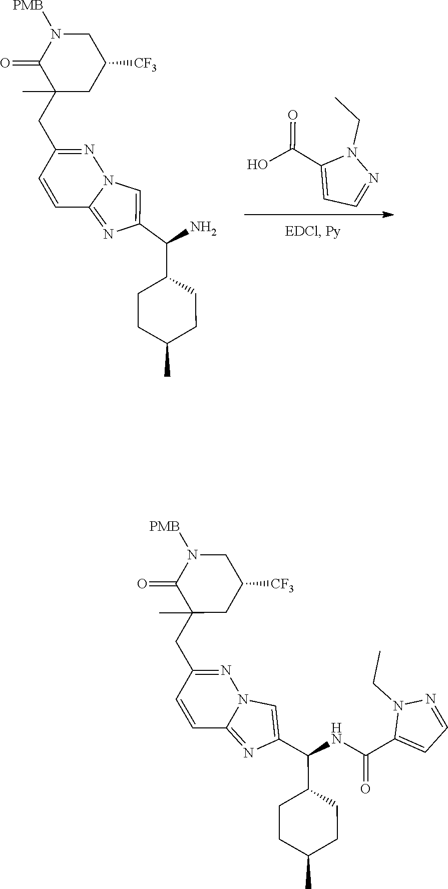

Intermediate 1-ethyl-N-((1S)-(6-(((5R)-1-(4-methoxybenzyl)-3-methyl-2-oxo-5-(trifluoromethyl)piperidin-3-yl)methyl)imidazo[1,2-b]pyridazin-2-yl)((1r,4S)-4-methylcyclohexyl)methyl)-1H-pyrazole-5-carboxamide was synthesized following General Procedure 4, using (5R)-3-((2-((S)-amino((1r,4S)-4-methylcyclohexyl)methyl)imidazo[1,2-b]pyridazin-6-yl)methyl)-1-(4-methoxybenzyl)-3-methyl-5-(trifluoromethyl)piperidin-2-one (90.0 mg, 161 μmol, 1.00 eq), 1-ethyl-1H-pyrazole-5-carboxylic acid (27.1 mg, 193 μmol, 1.20 eq), and EDCI (61.8 mg, 322 μmol, 2.00 eq), stirring for 2 h. The residue was purified by prep-TLC (SiO₂, DCM:MeOH=10:1, R_{f}(P1)=0.55). The product, 1-ethyl-N-((1S)-(6-(((5R)-1-(4-methoxybenzyl)-3-methyl-2-oxo-5-(trifluoromethyl)piperidin-3-yl)methyl)imidazo[1,2-b]pyridazin-2-yl)((1r,4S)-4-methylcyclohexyl)methyl)-1H-pyrazole-5-carboxamide (55.0 mg, 80.9 μmol, 50% yield), was obtained as a yellow solid, which was used in the next step without further purification.

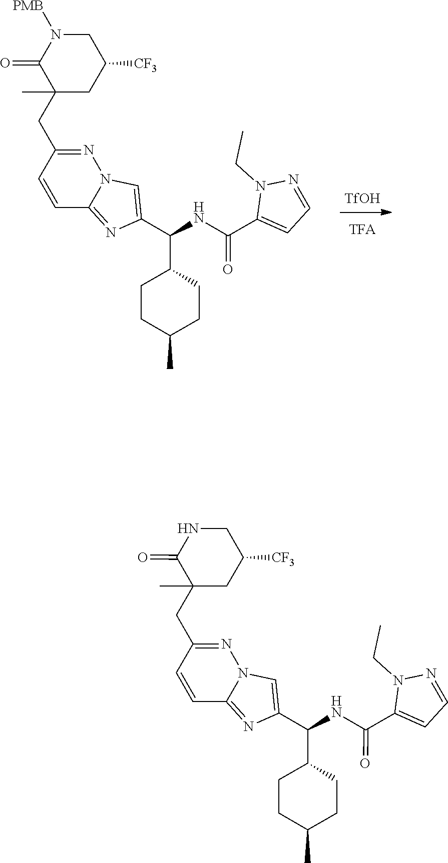

To a solution of 1-ethyl-N-((1S)-(6-(((5R)-1-(4-methoxybenzyl)-3-methyl-2-oxo-5-(trifluoromethyl)piperidin-3-yl)methyl)imidazo[1,2-b]pyridazin-2-yl)((1r,4S)-4-methylcyclohexyl)methyl)-1H-pyrazole-5-carboxamide (50.0 mg, 73.5 μmol, 1.00 eq) in TFA (0.500 mL) was added triflic acid (55.1 mg, 367 μmol, 32.5 μL, 5.00 eq). The reaction mixture was stirred at 50° C. for 1 h. The reaction mixture was concentrated under reduced pressure to give a residue. The residue was purified by prep-TLC (SiO2, DCM:MeOH=10:1, R_{f}=0.6). The mixture of stereoisomers, 1-ethyl-N-((1S)-(6-(((5R)-3-methyl-2-oxo-5-(trifluoromethyl)piperidin-3-yl)methyl)imidazo[1,2-b]pyridazin-2-yl)((1r,4S)-4-methylcyclohexyl)methyl)-1H-pyrazole-5-carboxamide (0.0300 g, 53.6 μmol, 73% yield), was obtained as a white solid. LCMS [M+H]⁺=560.4 m/z. Mixture of stereoisomers of 1-ethyl-N-((1S)-(6-(((5R)-3-methyl-2-oxo-5-(trifluoromethyl)piperidin-3-yl)methyl)imidazo[1,2-b]pyridazin-2-yl)((1r,4S)-4-methylcyclohexyl)methyl)-1H-pyrazole-5-carboxamide was purified by SFC (condition: REGIS(S, S) WHELK-O1 (250 mm×25 mm, 10 μm); mobile phase: [CO2-I-PrOH (0.1% NH₃H₂O)]; B %: 45%, isocratic elution mode). The first eluting, single stereoisomer of 1-ethyl-N-((1S)-(6-(((5R)-3-methyl-2-oxo-5-(trifluoromethyl)piperidin-3-yl)methyl)imidazo[1,2-b]pyridazin-2-yl)((1r,4S)-4-methylcyclohexyl)methyl)-1H-pyrazole-5-carboxamide (Compound 67, 11.6 mg, 17.8 μmol) was obtained as a white solid. LCMS [M+H]+=560.4 m/z.

### Example 68: Preparation of Compounds 68 and 69

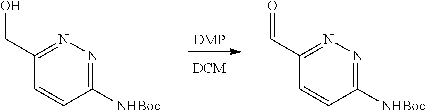 [SMILES: CC(C)(C)OC(=O)Nc1ccc(CO)nn1 ; CC(C)(C)OC(=O)Nc1ccc(C=O)nn1]

To a solution of tert-butyl (6-(hydroxymethyl)pyridazin-3-yl)carbamate (4.00 g, 17.7 mmol, 1.00 eq) in DCM (10.0 mL) was added DMP (15.0 g, 35.5 mmol, 11.0 mL, 2.00 eq) at 0° C. The reaction mixture was stirred at RT for 5 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The residue was purified by column chromatography (SiO₂, PE:EtOAc=1:0 to 0:1). The product, tert-butyl (6-formylpyridazin-3-yl)carbamate (3.96 g, crude), was obtained as a white solid and used in the next step without further purification.

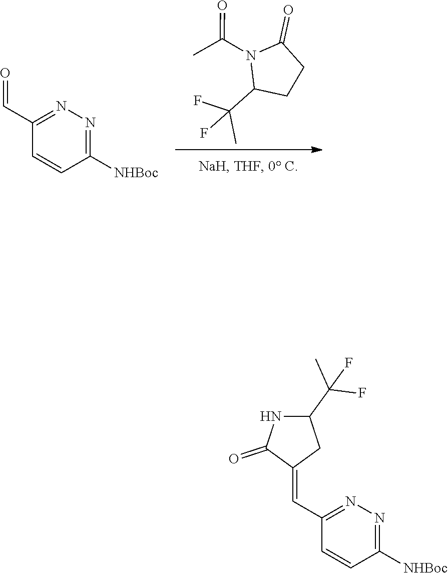 [SMILES: CC(C)(C)OC(=O)Nc1ccc(C=O)nn1 ; CC(=O)N1C(=O)CCC1C(C)(F)F ; CC(C)(C)OC(=O)Nc1ccc(/C=C2\CC(C(C)(F)F)NC2=O)nn1]

To a solution of 1-acetyl-5-(1,1-difluoroethyl)pyrrolidin-2-one (1.90 g, 9.94 mmol, 1.00 eq) in THF (20.0 mL) was added NaH (1.59 g, 39.7 mmol, 60.0% purity, 4.00 eq) at 0° C. After 30 min, to the reaction mixture was added tert-butyl (6-formylpyridazin-3-yl)carbamate (2.00 g crude) in THF (5.00 mL) stirred at RT for 2 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The residue was purified by column chromatography (SiO₂, PE:EtOAc=1/0 to 0/1). The product, tert-butyl (E)-(6-((5-(1,1-difluoroethyl)-2-oxopyrrolidin-3-ylidene)methyl)pyridazin-3-yl)carbamate (1.50 g, crude), was obtained as a white solid. LCMS [M+H]⁺=355.2 m/z.

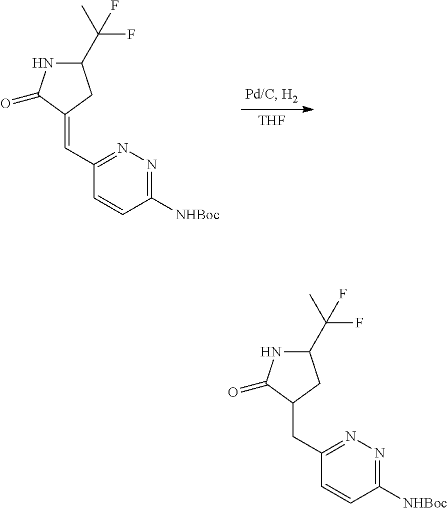 [SMILES: CC(C)(C)OC(=O)Nc1ccc(/C=C2\CC(C(C)(F)F)NC2=O)nn1 ; CC(C)(C)OC(=O)Nc1ccc(CC2CC(C(C)(F)F)NC2=O)nn1 ; "THF"]

To a solution of tert-butyl (E)-(6-((5-(1,1-difluoroethyl)-2-oxopyrrolidin-3-ylidene)methyl)pyridazin-3-yl)carbamate (1.00 g, crude) in THF (10.0 mL) was added Pd/C (300 mg, 10% purity). The reaction mixture was stirred at RT for 10 h under H₂. The reaction mixture was filtered and concentrated under reduced pressure to give a residue. The crude product was purified by prep-HPLC (HPLC: EW33907-383-P1A1) (Phenomenex luna C18 150×40 mm×15 um; mobile phase: [water (TFA)-ACN]; B %: 20%-50%, 10 min). The product, tert-butyl (6-((5-(1,1-difluoroethyl)-2-oxopyrrolidin-3-yl)methyl)pyridazin-3-yl)carbamate (0.300 g, crude), was obtained as a white solid. LCMS [M+H]⁺=357.2 m/z.

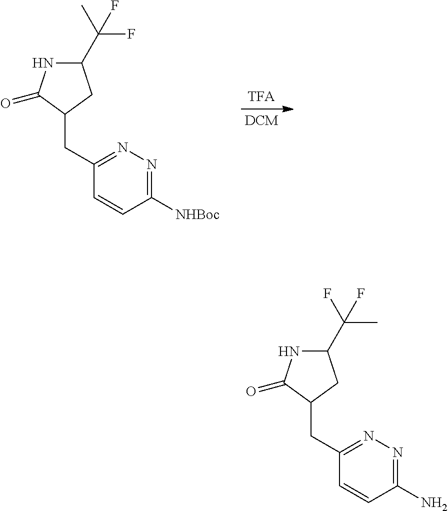

To a solution of tert-butyl (6-((5-(1,1-difluoroethyl)-2-oxopyrrolidin-3-yl)methyl)pyridazin-3-yl)carbamate (300 mg, crude) in DCM (1.00 mL) was added TFA (95.9 mg, 841 μmol, 62.5 μL, 1.00 eq). The reaction mixture was stirred at RT for 1 h. The product, 3-((6-aminopyridazin-3-yl)methyl)-5-(1,1-difluoroethyl)pyrrolidin-2-one (311 mg, crude, TFA salt) was obtained as a white solid. LCMS [M+H]⁺=257.2 m/z.

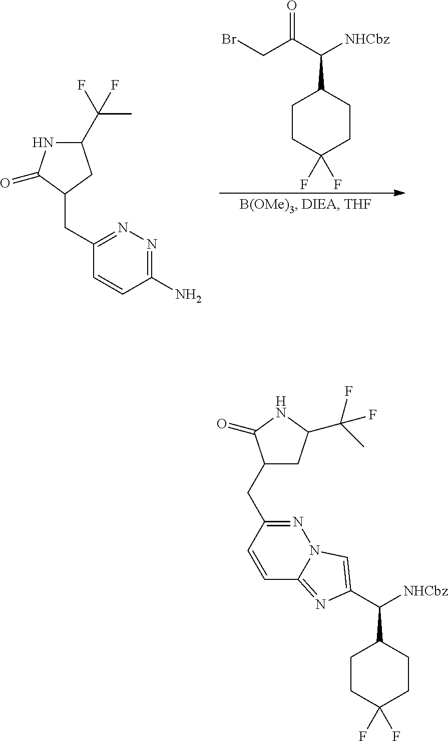 [SMILES: CC(F)(F)C1CC(Cc2ccc(N)nn2)C(=O)N1 ; O=C(N[C@H](C(=O)CBr)C1CCC(F)(F)CC1)OCc1ccccc1 ; "B(OMe)3, DIEA, THF" ; CC(F)(F)C1CC(Cc2ccc3nc([C@@H](NC(=O)OCc4ccccc4)C4CCC(F)(F)CC4)cn3n2)C(=O)N1]

Benzyl ((1S)-(4,4-difluorocyclohexyl)(6-((5-(1,1-difluoroethyl)-2-oxopyrrolidin-3-yl)methyl)imidazo[1,2-b]pyridazin-2-yl)methyl)carbamate was synthesized following General Procedure 1, using 3-((6-aminopyridazin-3-yl)methyl)-5-(1,1-difluoroethyl)pyrrolidin-2-one (274 mg, crude, TFA salt), Intermediate 2 (358 mg, 887 μmol, 1.20 eq), B(OMe)₃ (768 mg, 7.40 mmol, 835 μL, 10.0 eq), and DIEA (956 mg, 7.40 mmol, 1.29 mL, 10.0 eq). After 30 min at 70° C., crude product was purified by column chromatography (SiO₂, PE:EtOAc=1/0 to 0/1). The product, benzyl ((1S)-(4,4-difluorocyclohexyl)(6-((5-(1,1-difluoroethyl)-2-oxopyrrolidin-3-yl)methyl)imidazo[1,2-b]pyridazin-2-yl)methyl)carbamate (240 mg, 427.4 μmol, 8% yield over 5 steps), was obtained as a white solid. LCMS [M+H]⁺=562.2 m/z.

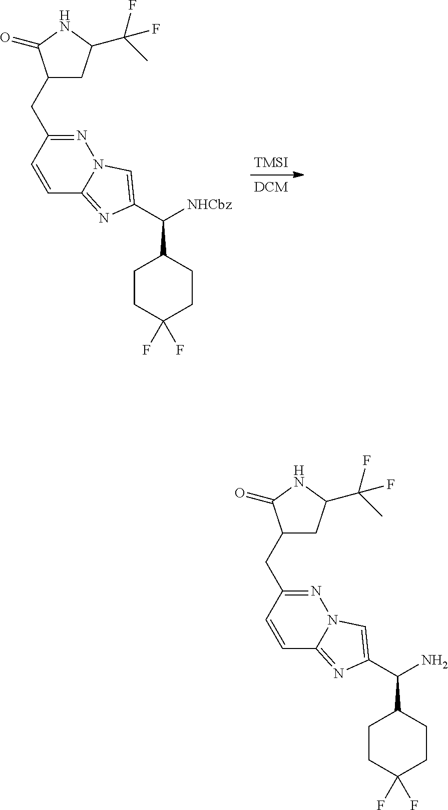 [SMILES: "TMSI" ; CC(F)(F)C1CC(Cc2ccc3nc([C@@H](NC(=O)OCc4ccccc4)C4CCC(F)(F)CC4)cn3n2)C(=O)N1 ; CC(F)(F)C1CC(Cc2ccc3nc([C@@H](N)C4CCC(F)(F)CC4)cn3n2)C(=O)N1]

Intermediate 3-((2-((S)-amino(4,4-difluorocyclohexyl)methyl)imidazo[1,2-b]pyridazin-6-yl)methyl)-5-(1,1-difluoroethyl)pyrrolidin-2-one was synthesized following General Procedure 2, using benzyl ((1S)-(4,4-difluorocyclohexyl)(6-((5-(1,1-difluoroethyl)-2-oxopyrrolidin-3-yl)methyl)imidazo[1,2-b]pyridazin-2-yl)methyl)carbamate (240 mg, 427 μmol, 1.00 eq) and TMSI (171 mg, 854 μmol, 116 μL, 2.00 eq). The product, 3-((2-((S)-amino(4,4-difluorocyclohexyl)methyl)imidazo[1,2-b]pyridazin-6-yl)methyl)-5-(1,1-difluoroethyl)pyrrolidin-2-one (200 mg, crude), was obtained as a white solid. LCMS [M+H]⁺=428.2 m/z.

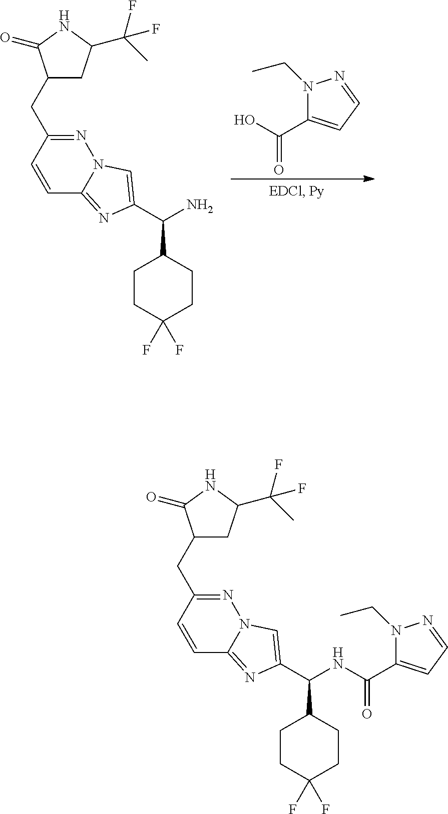

Intermediate N-((1S)-(4,4-difluorocyclohexyl)(6-((5-(1,1-difluoroethyl)-2-oxopyrrolidin-3-yl)methyl)imidazo[1,2-b]pyridazin-2-yl)methyl)-1-ethyl-1H-pyrazole-5-carboxamide was synthesized following General Procedure 4, using 3-((2-((S)-amino(4,4-difluorocyclohexyl)methyl)imidazo[1,2-b]pyridazin-6-yl)methyl)-5-(1,1-difluoroethyl)pyrrolidin-2-one (180 mg, crude), 1-ethyl-1H-pyrazole-5-carboxylic acid (70.8 mg 505 μmol, 1.20 eq), and EDCI (242 mg, 1.26 mmol, 3.00 eq), stirring for 1 h. The residue was purified by prep-TLC (SiO₂, DCM:MeOH=10/1). The product, N-((1S)-(4,4-difluorocyclohexyl)(6-((5-(1,1-difluoroethyl)-2-oxopyrrolidin-3-yl)methyl)imidazo[1,2-b]pyridazin-2-yl)methyl)-1-ethyl-1H-pyrazole-5-carboxamide (150 mg, crude), was obtained as a white solid. LCMS [M+H]⁺=550.3 m/z. The crude product was purified by prep-SFC (EW33907-384-P1A) (REGIS (R, R) WHELKO1 (250 mm×25 mm, 10 μm); mobile phase: [ACN/IPA (0.1% NH₃H₂O)]; B %: 30%-30%, 7.0 min). The first and second peaks coeluted to give a mixture of two stereoisomers of N-((1S)-(4,4-difluorocyclohexyl)(6-((5-(1,1-difluoroethyl)-2-oxopyrrolidin-3-yl)methyl)imidazo[1,2-b]pyridazin-2-yl)methyl)-1-ethyl-1H-pyrazole-5-carboxamide (Compound 68, 5.23 mg, 9.50 μmol, 2.5% yield over 3 steps) was obtained as a white solid. LCMS [M+H]⁺=550.2 m/z. The fourth eluting, single stereoisomer of N-((1S)-(4,4-difluorocyclohexyl)(6-((5-(1,1-difluoroethyl)-2-oxopyrrolidin-3-yl)methyl)imidazo[1,2-b]pyridazin-2-yl)methyl)-1-ethyl-1H-pyrazole-5-carboxamide (Compound 69, 34.7 mg, 60.2 mol, 16% yield over 3 steps) was obtained as a white solid. LCMS [M+H]⁺=550.2 m/z.

### Example 69: Preparation of Compounds 70 and 71

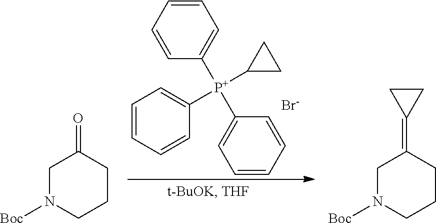 [SMILES: CC(C)(C)OC(=O)N1CCCC(=O)C1 ; c1ccc([P+](c2ccccc2)(c2ccccc2)C2CC2)cc1 ; [Br-] ; CC(C)(C)OC(=O)N1CCCC(=C2CC2)C1]

To a solution of cyclopropyltriphenylphosphonium bromide (11.5 g, 30.1 mmol, 1.20 eq) in THE (150 mL) was added t-BuOK (1.00 M, 50.1 mL, 2.00 eq) at 0° C. and stirred for 30 min. The reaction mixture was added tert-butyl 3-oxopiperidine-1-carboxylate (5.00 g, 25.0 mmol, 1.00 eq) in THF (50.0 mL). The reaction mixture was stirred at RT for 10 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The residue was purified by column chromatography (SiO₂, PE:EtOAc=1/0 to 0/1). The product, tert-butyl 3-cyclopropylidenepiperidine-1-carboxylate (3.80 g, 17.02 mmol, 68% yield), was obtained as a white solid. ¹H NMR (400 MHz, CDCl₃) 6.4.02 (s, 2H), 3.48 (t, J=4.0 Hz, 2H), 2.38 (t, J=4.0 Hz, 2H), 1.74-1.58 (m, 2H), 1.45 (s, 9H), 1.12-1.00 (m, 4H).

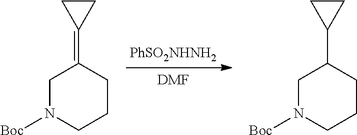

To a solution of tert-butyl 3-cyclopropylidenepiperidine-1-carboxylate (1.00 g, 4.48 mmol, 1.00 eq) in DMF (10.0 mL) was added benzenesulfonohydrazide (3.08 g, 17.9 mmol, 4.00 eq). The reaction mixture was stirred at 100° C. for 5 h under N₂. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The residue was purified by column chromatography (SiO₂, PE:EtOAc=1/0 to 0/1). The product, tert-butyl 3-cyclopropylpiperidine-1-carboxylate (0.600 g, crude), was obtained as a white solid, which was used directly in the next step without further purification.

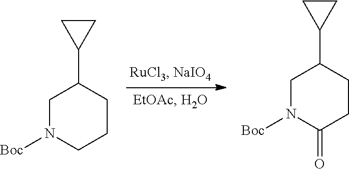

To a solution of tert-butyl 3-cyclopropylpiperidine-1-carboxylate (300 mg, crude) in EtOAc (6.00 mL) and H₂O (1.50 mL) was added NaIO₄ (1.42 g, 6.66 mmol, 368 μL, 5.00 eq) and RuCl₃ (138 mg, 665 μmol, 44.4 μL, 0.500 eq) at 0° C. The reaction mixture was stirred at RT for 1 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The residue was purified by column chromatography (SiO₂, PE:EtOAc=1/0 to 0/1). The product, tert-butyl 5-cyclopropyl-2-oxopiperidine-1-carboxylate (180 mg, 752.1 μmol, 34% yield over 2 steps), was obtained as a white solid. ¹H NMR (400 MHz, CDCl₃) δ 3.92-3.81 (m, 1H), 3.39-3.30 (m, 1H), 2.66-2.57 (m, 1H), 2.50-2.35 (m, 1H), 2.12-1.86 (m, 2H), 1.65-1.60 (m, 1H), 1.56-1.51 (m, 9H), 1.15-1.01 (m, 1H), 0.83-0.54 (m, 2H), 0.23-0.14 (m, 2H).

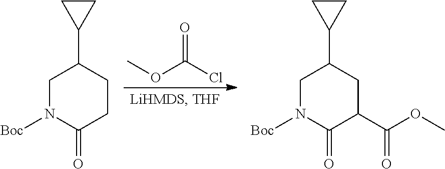

To a solution of tert-butyl 5-cyclopropyl-2-oxopiperidine-1-carboxylate (175 mg, 731 μmol, 1.00 eq) in THF (5.00 mL) was added LiHMDS (1 M, 1.46 mL, 2.00 eq) at −70° C. and stirred for 30 min. The reaction mixture was added methyl chloroformate (0.370 g, 3.97 mmol, 306 μL, 5.43 eq). The reaction was stirred at −70° C. for 2 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The product, 1-(tert-butyl) 3-methyl 5-cyclopropyl-2-oxopiperidine-1,3-dicarboxylate (217 mg, 730 μmol, quantitative), was obtained as a white solid. LCMS [M−55]⁺=242.1 m/z.

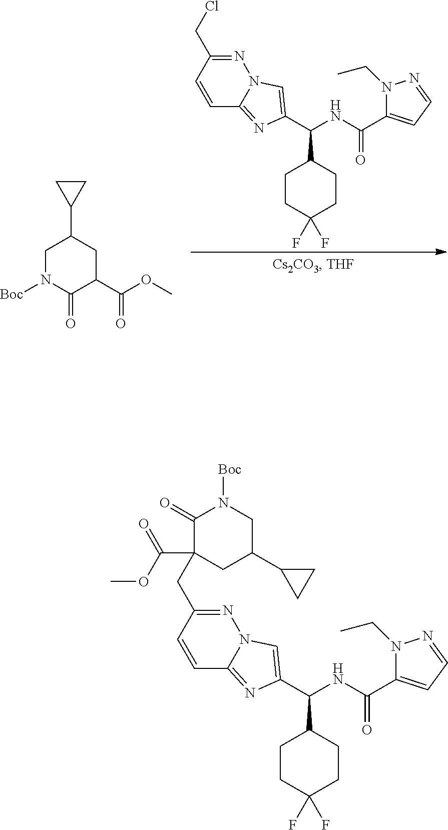 [SMILES: COC(=O)C1CC(C2CC2)CN(C(=O)OC(C)(C)C)C1=O ; CCn1nccc1C(=O)N[C@H](c1cn2nc(CCl)ccc2n1)C1CCC(F)(F)CC1 ; CCn1nccc1C(=O)N[C@H](c1cn2nc(CC3(C(=O)OC)CC(C4CC4)CN(C(=O)OC(C)(C)C)C3=O)ccc2n1)C1CCC(F)(F)CC1]

To a solution of (S)—N-((6-(chloromethyl)imidazo[1,2-b]pyridazin-2-yl)(4,4-difluorocyclohexyl)methyl)-1-ethyl-1H-pyrazole-5-carboxamide (0.300 g, 686 μmol, 1.00 eq) and 1-(tert-butyl) 3-methyl 5-cyclopropyl-2-oxopiperidine-1,3-dicarboxylate (204 mg, 686 μmol, 1.00 eq) in DMF (5.00 mL) was added Cs₂CO₃ (671 mg, 2.06 mmol, 3.00 eq). The reaction mixture was stirred at 50° C. for 1 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The crude product was purified by prep-HPLC (Phenomenex luna C18 150×25 mm×10 μm; mobile phase: [water (FA)-ACN]; gradient: 52%-82% B over 10 min). The product, 1-(tert-butyl) 3-methyl 5-cyclopropyl-3-((2-((S)-(4,4-difluorocyclohexyl)(1-ethyl-1H-pyrazole-5-carboxamido)methyl)imidazo[1,2-b]pyridazin-6-yl)methyl)-2-oxopiperidine-1,3-dicarboxylate (0.200 g), was obtained as a white solid. LCMS [M+H]⁺=698.3 m/z.

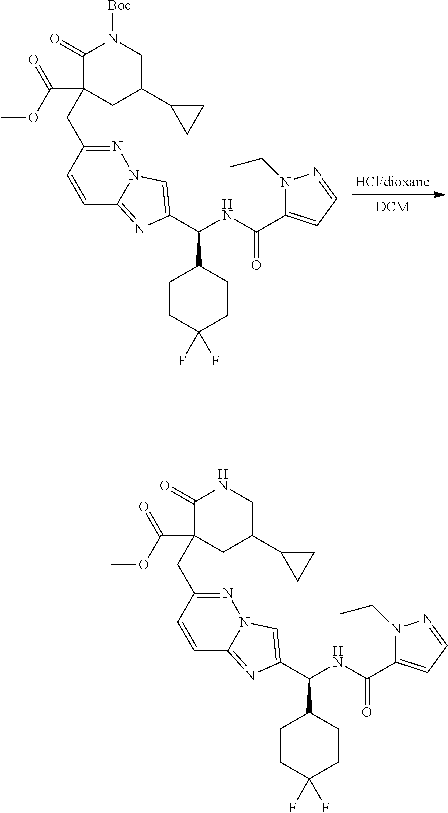 [SMILES: CCn1nccc1C(=O)N[C@H](c1cn2nc(CC3(C(=O)OC)CC(C4CC4)CN(C(=O)OC(C)(C)C)C3=O)ccc2n1)C1CCC(F)(F)CC1 ; CCn1nccc1C(=O)N[C@H](c1cn2nc(CC3(C(=O)OC)CC(C4CC4)CNC3=O)ccc2n1)C1CCC(F)(F)CC1]

To a solution of 1-(tert-butyl) 3-methyl 5-cyclopropyl-3-((2-((S)-(4,4-difluorocyclohexyl)(1-ethyl-1H-pyrazole-5-carboxamido)methyl)imidazo[1,2-b]pyridazin-6-yl)methyl)-2-oxopiperidine-1,3-dicarboxylate (200 mg, 286 μmol, 1.00 eq) in DCM (2.00 mL) was added HCl/dioxane (4 M, 358 μL, 5.00 eq). The reaction mixture was stirred at RT for 30 min The reaction mixture was filtered and concentrated under reduced pressure to give a residue. The product, methyl 5-cyclopropyl-3-((2-((S)-(4,4-difluorocyclohexyl)(1-ethyl-1H-pyrazole-5-carboxamido)methyl)imidazo[1,2-b]pyridazin-6-yl)methyl)-2-oxopiperidine-3-carboxylate (171 mg, 269.7 μmol, HCl salt, 39% yield over 2 steps), was obtained as a white solid. LCMS [M+H]⁺=598.3 m/z.

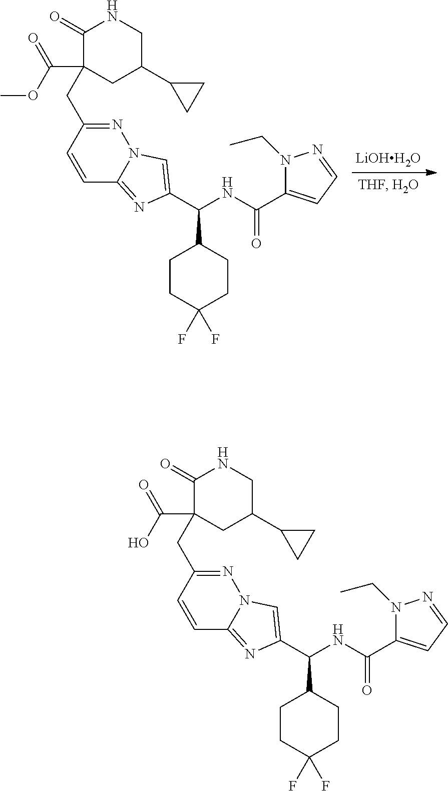

To a solution of methyl 5-cyclopropyl-3-((2-((S)-(4,4-difluorocyclohexyl)(1-ethyl-1H-pyrazole-5-carboxamido)methyl)imidazo[1,2-b]pyridazin-6-yl)methyl)-2-oxopiperidine-3-carboxylate (171 mg, 269.7 μmol, 1.00 eq) in THF (3.00 mL) was added LiOH·H₂O (60.0 mg, 1.43 mmol, 5.30 eq) in H₂O (1.00 mL). The reaction mixture was stirred at RT for 2 h. The reaction mixture was washed with EtOAc (20.0 mL×2). The pH of the aqueous phase was adjusted to 3 with 1 M HCl and extracted with EtOAc (20.0 mL×2). The combined organic layers were dried over Na₂SO₄, filtered, and concentrated under reduced pressure to give a residue. The product, 5-cyclopropyl-3-((2-((S)-(4,4-difluorocyclohexyl)(1-ethyl-1H-pyrazole-5-carboxamido)methyl)imidazo[1,2-b]pyridazin-6-yl)methyl)-2-oxopiperidine-3-carboxylic acid (166 mg, crude), was obtained as a white solid. LCMS [M+H]⁺=584.3 m/z.

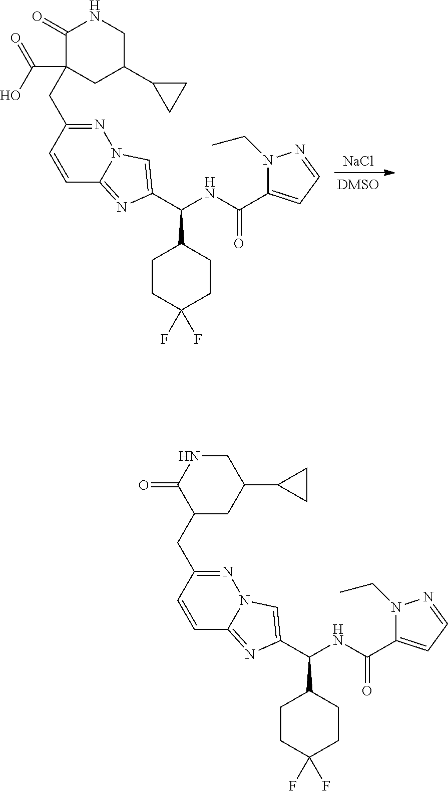

To a solution of 5-cyclopropyl-3-((2-((S)-(4,4-difluorocyclohexyl)(1-ethyl-1H-pyrazole-5-carboxamido)methyl)imidazo[1,2-b]pyridazin-6-yl)methyl)-2-oxopiperidine-3-carboxylic acid (166 mg, 284 μmol, 1.00 eq) in DMSO (2.00 mL) was added NaCl (49.8 mg, 853 μmol, 3.00 eq). The reaction mixture was stirred at 120° C. for 1 h. The reaction mixture was diluted with H₂O (40.0 mL) and extracted with DCM (40.0 mL×3). The combined organic layers were washed with brine (40.0 mL×3), dried over Na₂SO₄, filtered, and concentrated under reduced pressure to give a residue. The residue was purified by prep-TLC (SiO₂, DCM:MeOH=10:1). The mixture of stereoisomers, N-((1S)-(6-((5-cyclopropyl-2-oxopiperidin-3-yl)methyl)imidazo[1,2-b]pyridazin-2-yl)(4,4-difluorocyclohexyl)methyl)-1-ethyl-1H-pyrazole-5-carboxamide (120 mg 222.4 μmol, 82% yield over 2 steps), was obtained as a white solid. LCMS [M+H]⁺=540.3 m/z. The mixture of stereoisomers of N-((1S)-(6-((5-cyclopropyl-2-oxopiperidin-3-yl)methyl)imidazo[1,2-b]pyridazin-2-yl)(4,4-difluorocyclohexyl)methyl)-1-ethyl-1H-pyrazole-5-carboxamide was purified by prep-SFC (EW33907-453-P1A1) (DAICEL CHIRALPAK IC (250 mm×30 mm, 10 μm); mobile phase: [CO₂-ACN/MeOH (0.1% NH₃H₂O)]; B %: 55%, isocratic elution mode) and (DAICEL CHIRALPAK AS (250 mm×30 mm, 10 μm); mobile phase: [CO₂-MeOH (0.1% NH₃H₂O)]; B %: 40%, isocratic elution mode). The second eluting single stereoisomer of N-((1S)-(6-((5-cyclopropyl-2-oxopiperidin-3-yl)methyl)imidazo[1,2-b]pyridazin-2-yl)(4,4-difluorocyclohexyl)methyl)-1-ethyl-1H-pyrazole-5-carboxamide (Compound 70, 8.88 mg, 16.3 μmol, 8.8% yield) was obtained as a yellow solid. LCMS [M+H]⁺=540.3 m/z. The third eluting, single stereoisomer of N-((1S)-(6-((5-cyclopropyl-2-oxopiperidin-3-yl)methyl)imidazo[1,2-b]pyridazin-2-yl)(4,4-difluorocyclohexyl)methyl)-1-ethyl-1H-pyrazole-5-carboxamide (Compound 71, 8.61 mg, 15.9 μmol, 8.6% yield) was obtained as a yellow solid. LCMS [M+H]⁺=540.3 m/z.

### Example 70: Preparation of Compound 72

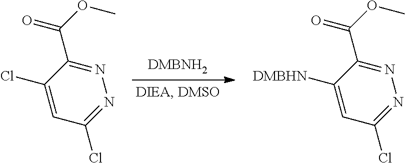

To a solution of methyl 4,6-dichloropyridazine-3-carboxylate (10.0 g, 48.3 mmol, 1.00 eq) in DMSO (90.0 mL) was added (3,4-dimethylphenyl)methanamine (9.69 g, 58.0 mmol, 8.73 mL, 1.20 eq) and DIEA (12.5 g, 96.6 mmol, 16.8 mL, 2.00 eq). The reaction mixture was stirred at 80° C. for 1 h. The residue was diluted with H₂O (150 mL) and extracted with DCM (150 mL×3). The combined organic layers were dried over Na₂SO₄, filtered, and concentrated under reduced pressure to give a residue. The residue was purified by column chromatography (1:1 PE:EtOAc) to afford methyl 6-chloro-4-((3,4-dimethylbenzyl)amino)pyridazine-3-carboxylate (13.6 g, 40.3 mmol, 83% yield) as a white solid. LCMS [M+H]⁺=338.0 m/z. ¹H NMR: (400 MHz, CDCl₃) δ 8.39 (br s, 1H), 7.11 (d, J=8.4 Hz, 1H), 6.81 (s, 1H), 6.50 (d, J=1.8 Hz, 1H), 6.46 (dd, J₁=8.4 Hz, J₂=2.0 Hz, 1H), 4.33 (d, J=5.8 Hz, 2H), 4.00 (s, 3H), 3.86 (s, 3H), 3.81 (s, 3H).

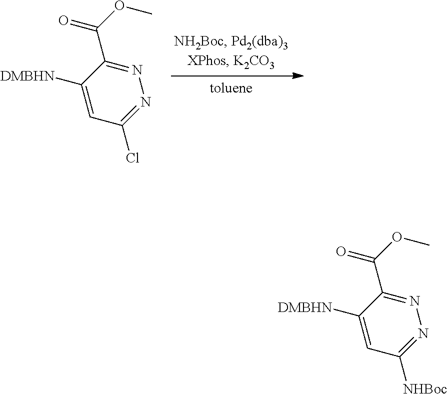

A mixture of methyl 6-chloro-4-((3,4-dimethylbenzyl)amino)pyridazine-3-carboxylate (13.6 g, 40.3 mmol, 1.00 eq), NH₂Boc (23.6 g, 201 mmol, 5.00 eq), K₂CO₃ (16.7 g, 121 mmol, 3.00 eq), XPhos (1.92 g, 4.03 mmol, 0.100 eq), and Pd₂(dba)₃ (3.69 g, 4.03 mmol, 0.100 eq) in toluene (200 mL) was degassed and purged with N₂ 3 times, and then the reaction mixture was stirred at 80° C. for 2 h under N₂ atmosphere. The residue was diluted with H₂O (200 mL) and extracted with DCM (200 mL×3). The combined organic layers were dried over Na₂SO₄, filtered, and concentrated under reduced pressure to give a residue. The residue was purified by column chromatography (1:1 PE:EtOAc) to afford methyl 6-((tert-butoxycarbonyl)amino)-4-((3,4-dimethylbenzyl)amino)pyridazine-3-carboxylate (6.00 g, 14.3 mmol, 36% yield) as a yellow solid. LCMS [M+H]⁺=419.0 m/z. ¹H NMR: (400 MHz, CDCl₃) δ 8.06-7.96 (m, 1H), 7.72 (br s, 1H), 6.94 (d, J=8.4 Hz, 1H), 6.25-6.15 (m, 2H), 4.11 (d, J=5.6 Hz, 2H), 3.69 (s, 3H), 3.59 (s, 3H), 3.54 (s, 3H), 1.27 (s, 9H).

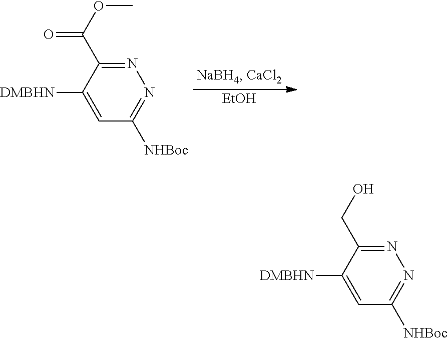

To a solution of methyl 6-((tert-butoxycarbonyl)amino)-4-((3,4-dimethylbenzyl)amino)pyridazine-3-carboxylate (6.00 g, 14.3 mmol, 1.00 eq) in EtOH (60.0 mL) was added NaBH₄ (2.98 g, 78.9 mmol, 5.50 eq) and CaCl₂ (796 mg, 7.17 mmol, 0.500 eq) at 0° C. The reaction mixture was stirred at RT for 2 h. The reaction mixture was treated with 150 mL of a saturated aqueous NH₄Cl solution at 0° C. and extracted with DCM (150 mL×3). The combined organic layers was dried over Na₂SO₄, filtered, and concentrated under reduced pressure to give a residue. The residue was purified by column chromatography (0-100% EtOAc in PE followed by 10:1 DCM:MeOH) to afford tert-butyl (5-((3,4-dimethylbenzyl)amino)-6-(hydroxymethyl)pyridazin-3-yl)carbamate (3.78 g, 9.68 mmol, 68% yield) as a yellow solid. LCMS [M+H]⁺=391.1 m/z.

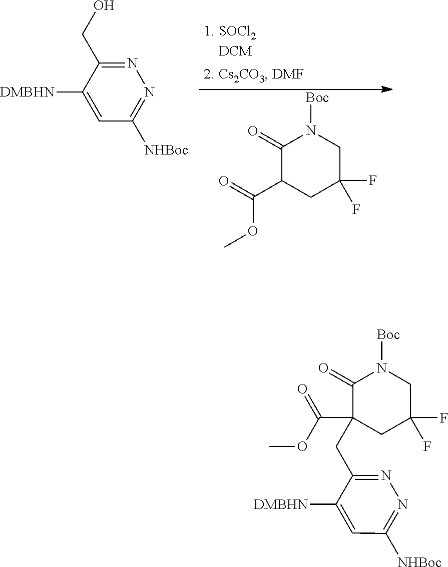

1-(tert-butyl) 3-methyl 3-((6-((tert-butoxycarbonyl)amino)-4-((2,4-dimethoxybenzyl)amino)pyridazin-3-yl)methyl)-5,5-difluoro-2-oxopiperidine-1,3-dicarboxylate was synthesized using General Procedure 7, employing tert-butyl (5-((3,4-dimethylbenzyl)amino)-6-(hydroxymethyl)pyridazin-3-yl)carbamate, and General Procedure 8, employing 1-(tert-butyl) 3-methyl 5,5-difluoro-2-oxopiperidine-1,3-dicarboxylate and tert-butyl (6-(chloromethyl)-5-((3,4-dimethylbenzyl)amino)pyridazin-3-yl)carbamate to afford 1-(tert-butyl) 3-methyl 3-((6-((tert-butoxycarbonyl)amino)-4-((2,4-dimethoxybenzyl)amino)pyridazin-3-yl)methyl)-5,5-difluoro-2-oxopiperidine-1,3-dicarboxylate (2.67 g, 4.01 mmol) as a white solid. LCMS [M+H]⁺=666.7 m/z.

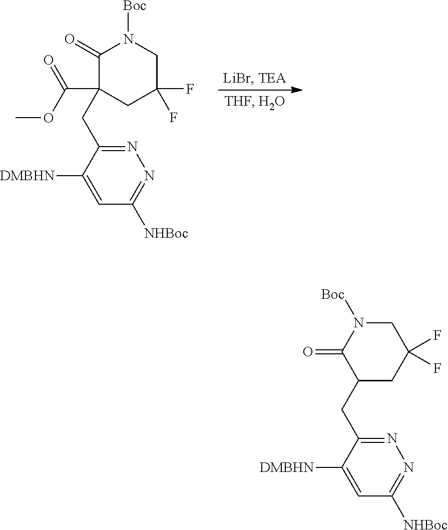

A solution of 1-(tert-butyl) 3-methyl 3-((6-((tert-butoxycarbonyl)amino)-4-((2,4-dimethoxybenzyl)amino)pyridazin-3-yl)methyl)-5,5-difluoro-2-oxopiperidine-1,3-dicarboxylate (1.00 g, 1.50 mmol, 1.00 eq) in THF (5.00 mL) and H₂O (5.00 mL) was added TEA (4.26 g, 42.1 mmol, 5.85 mL, 28.0 eq) and LiBr (4.70 g, 54.1 mmol, 1.36 mL, 36.0 eq). The reaction mixture was stirred at 60° C. for 48 h. The residue was diluted with H₂O (100 mL) and extracted with DCM (100 mL×3). The combined organic layers were dried over Na₂SO₄, filtered, and concentrated under reduced pressure to afford tert-butyl 3-((6-((tert-butoxycarbonyl)amino)-4-((2,4-dimethoxybenzyl)amino)pyridazin-3-yl)methyl)-5,5-difluoro-2-oxopiperidine-1-carboxylate (1.10 g, crude) as a white solid. LCMS [M+H]⁺=608.3 m/z.

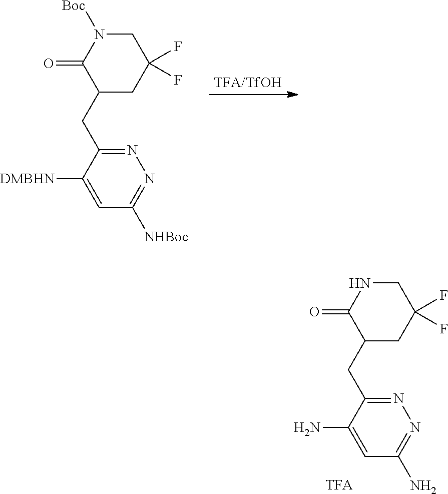

A solution of tert-butyl 3-((6-((tert-butoxycarbonyl)amino)-4-((2,4-dimethoxybenzyl)amino)pyridazin-3-yl)methyl)-5,5-difluoro-2-oxopiperidine-1-carboxylate (1.10 g, 1.81 mmol, 1.00 eq) in TFA (10.0 mL) was added TfOH (425 mg, 2.83 mmol, 0.25 mL, 1.56 eq). The reaction mixture was stirred at 90° C. for 30 min. The reaction mixture was concentrated under the vacuum to afford 3-((4,6-diaminopyridazin-3-yl)methyl)-5,5-difluoropiperidin-2-one (670 mg, 1.80 mmol, quantitative, TFA salt) as a red oil. LCMS [M+H]⁺=258.0 m/z.

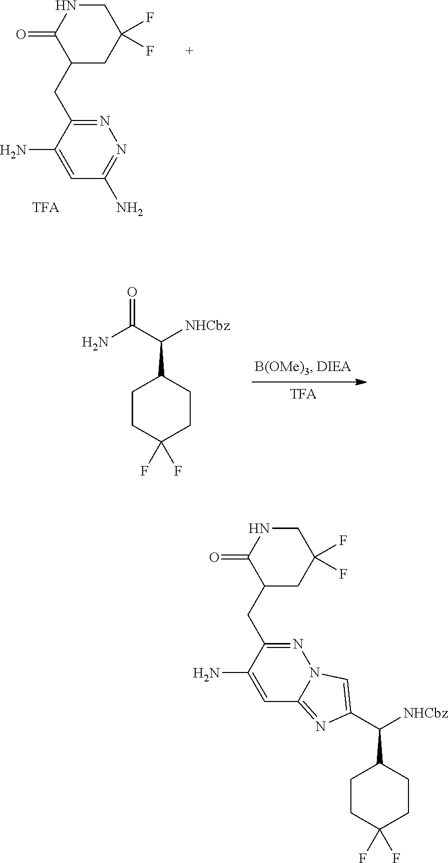

Benzyl ((1S)-(7-amino-6-((5,5-difluoro-2-oxopiperidin-3-yl)methyl)imidazo[1,2-b]pyridazin-2-yl)(4,4-difluorocyclohexyl)methyl)carbamate (590 mg, 1.05 mmol, yellow solid) was synthesized using General Procedure 1, employing benzyl (S)-(2-amino-1-(4,4-difluorocyclohexyl)-2-oxoethyl)carbamate and 3-((4,6-diaminopyridazin-3-yl)methyl)-5,5-difluoropiperidin-2-one (TFA salt).

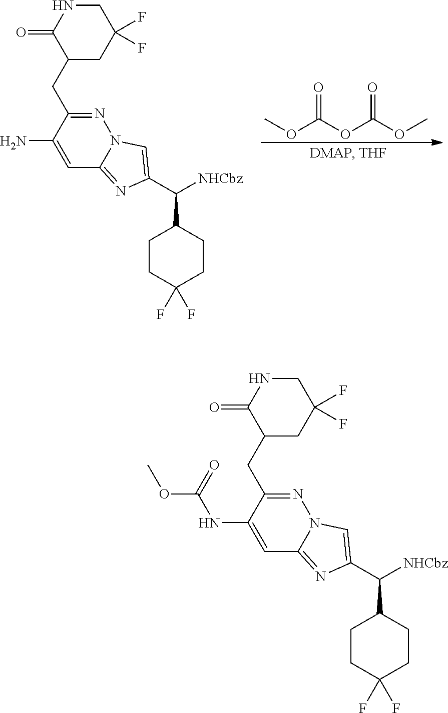

To a solution of benzyl ((1S)-(7-amino-6-((5,5-difluoro-2-oxopiperidin-3-yl)methyl)imidazo[1,2-b]pyridazin-2-yl)(4,4-difluorocyclohexyl)methyl)carbamate (100 mg 177 μmol, 1.00 eq) and dimethyl dicarbonate (71.5 mg, 533 μmol, 57.2 μL, 3.00 eq) in THF (5.00 mL) was added DMAP (4.34 mg, 35.5 μmol, 0.200 eq). The reaction mixture was stirred at 70° C. for 12 h. The reaction mixture was diluted with water (30 mL), extracted with EtOAc (30 mL×3), dried over anhydrous Na₂SO₄, filtrated, and concentrated under reduced pressure. The residue was purified by prep-TLC (SiO₂, EtOAc) to afford benzyl ((1S)-(6-((5,5-difluoro-2-oxopiperidin-3-yl)methyl)-7-((methoxycarbonyl)amino)imidazo[1,2-b]pyridazin-2-yl)(4,4-difluorocyclohexyl)methyl)carbamate (15.0 mg, 24.1 μmol, 14% yield) as a white solid. LCMS [M+H]⁺=621.1 m/z.

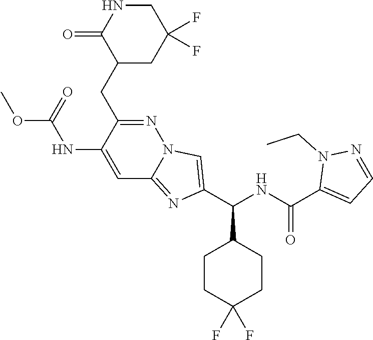 [SMILES: CCn1nccc1C(=O)N[C@H](c1cn2nc(CC3CC(F)(F)CNC3=O)c(NC(=O)OC)cc2n1)C1CCC(F)(F)CC1]

The title compound was synthesized according to General Procedure 3, employing benzyl ((1S)-(6-((5,5-difluoro-2-oxopiperidin-3-yl)methyl)-7-((methoxycarbonyl)amino)imidazo[1,2-b]pyridazin-2-yl)(4,4-difluorocyclohexyl)methyl)carbamate, and General Procedure 4, employing methyl (2-((S)-amino(4,4-difluorocyclohexyl)methyl)-6-((5,5-difluoro-2-oxopiperidin-3-yl)methyl)imidazo[1,2-b]pyridazin-7-yl)carbamate and 1-ethyl-1H-pyrazole-5-carboxylic acid to afford Compound 72, methyl (6-((5,5-difluoro-2-oxopiperidin-3-yl)methyl)-2-((S)-(4,4-difluorocyclohexyl)(1-ethyl-1H-pyrazole-5-carboxamido)methyl)imidazo[1,2-b]pyridazin-7-yl)carbamate, (6.95 mg, 10.10 μmol) as a white solid. LCMS [M+H]⁺=609.2 m/z.

### Example 71: Preparation of Compounds 73 and 74

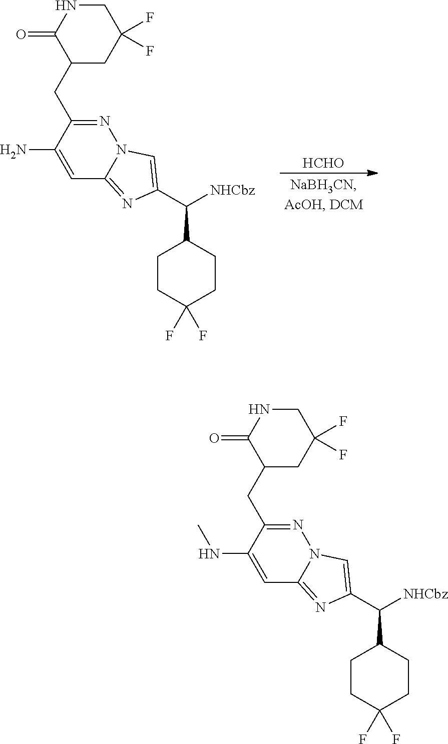 [SMILES: Nc1cc2nc([C@@H](NC(=O)OCc3ccccc3)C3CCC(F)(F)CC3)cn2nc1CC1CC(F)(F)CNC1=O ; "HCHO" ; CNc1cc2nc([C@@H](NC(=O)OCc3ccccc3)C3CCC(F)(F)CC3)cn2nc1CC1CC(F)(F)CNC1=O]

A mixture of benzyl ((1S)-(7-amino-6-((5,5-difluoro-2-oxopiperidin-3-yl)methyl)imidazo[1,2-b]pyridazin-2-yl)(4,4-difluorocyclohexyl)methyl)carbamate (200 mg 355 μmol, 1.00 eq), HCHO (288 mg, 3.56 mmol, 264 μL, 37% purity, 10.0 eq), NaBH₃CN (89.3 mg 1.42 mmol, 4.00 eq), and AcOH (21.3 mg, 355 μmol, 20.3 μL, 1.00 eq) in DCM (5 mL) was degassed and purged with N₂ 3 times, and then the reaction mixture was stirred at RT for 2 h under N₂ atmosphere. The reaction mixture was diluted with H₂O (30.0 mL) and extracted with EtOAc (30.0 mL×3), dried with anhydrous Na₂SO₄, filtrated, and concentrated under reduced pressure. The residue was purified by prep-TLC (SiO₂, EtOAc) to afford benzyl ((1S)-(6-((5,5-difluoro-2-oxopiperidin-3-yl)methyl)-7-(methylamino)imidazo[1,2-b]pyridazin-2-yl)(4,4-difluorocyclohexyl)methyl)carbamate (30.0 mg, 52.0 μmol, 15% yield) as a yellow solid. LCMS [M+H]⁺=577.2 m/z.

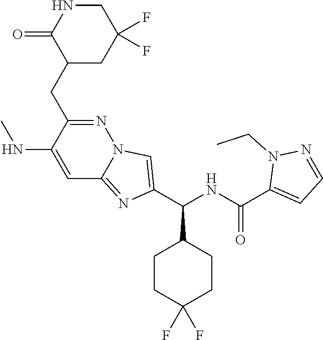 [SMILES: CCn1nccc1C(=O)N[C@H](c1cn2nc(CC3CC(F)(F)CNC3=O)c(NC)cc2n1)C1CCC(F)(F)CC1]

The title compound was prepared using General Procedure 2, employing benzyl ((1S)-(6-((5,5-difluoro-2-oxopiperidin-3-yl)methyl)-7-(methylamino)imidazo[1,2-b]pyridazin-2-yl)(4,4-difluorocyclohexyl)methyl)carbamate, and General Procedure 4, employing 3-((2-((S)-amino(4,4-difluorocyclohexyl)methyl)-7-(methylamino)imidazo[1,2-b]pyridazin-6-yl)methyl)-5,5-difluoropiperidin-2-one and 1-ethyl-1H-pyrazole-5-carboxylic acid to afford N-((1S)-(6-((5,5-difluoro-2-oxopiperidin-3-yl)methyl)-7-(methylamino)imidazo[1,2-b]pyridazin-2-yl)(4,4-difluorocyclohexyl)methyl)-1-ethyl-1H-pyrazole-5-carboxamide, which was further purified by chiral prep-SFC (DAICEL CHIRALCEL OD-H (250 mm×30 mm, 5 um); 25% mobile phase: [0.1% NH₃H₂O MeOH]) to afford Compound 74 as the first eluting, single stereoisomer (6.29 mg, 10.33 μmol, LCMS [M+H]⁺=565.2 m z) and gray solid. Compound 73 was isolated as the second eluting, single stereoisomer (10.32 mg, 15.12 μmol, LCMS [M+H]⁺=565.2 m z) and yellow solid.

### Example 72: Preparation of Compound 75

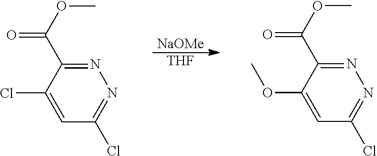

To a solution of methyl 4,6-dichloropyridazine-3-carboxylate (12.0 g, 57.9 mmol, 1.00 eq) in THE (100 mL) was added NaOMe (9.39 g, 52.1 mmol, 30.0% purity, 0.900 eq) at 0° C. The reaction mixture was stirred at RT for 15 h. The reaction mixture was diluted with 1.0 M HCl (10.0 mL) at 0° C. and adjusted with saturated aqueous NaHCO₃ to pH=8, then extracted with EtOAc (30.0 mL×2). The combined organic layers were washed with H₂O, dried over Na₂SO₄, filtered, and concentrated under reduced pressure to give a residue. The residue was purified by column chromatography (SiO₂, 30:1 to 1:1 PE/EtOAc) to afford methyl 6-chloro-4-methoxypyridazine-3-carboxylate (6.00 g, 29.6 mmol, 51% yield) as white solid. ¹H NMR: (400 MHz, CDCl₃) δ 7.08 (s, 1H), 4.01 (s, 3H), 4.00 (s, 3H).

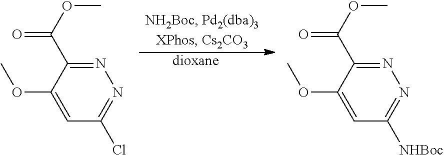 [SMILES: COC(=O)c1nnc(Cl)cc1OC ; "dioxane" ; "NH2Boc, Pd2(dba)3XPhos, Cs2CO3" ; COC(=O)c1nnc(NC(=O)OC(C)(C)C)cc1OC]

To a solution of methyl 6-chloro-4-methoxypyridazine-3-carboxylate (6.00 g, 29.6 mmol, 1.00 eq) and NH₂Boc (5.20 g, 44.4 mmol, 1.50 eq) in dioxane (100 mL) was added Cs₂CO₃ (28.9 g, 88.8 mmol, 3.00 eq), XPhos (2.82 g, 5.92 mmol, 0.200 eq), and Pd₂(dba)₃ (2.71 g, 2.96 mmol, 0.100 eq). The reaction mixture was stirred at 100° C. for 10 h under N₂. The reaction mixture was filtered with EtOAc (600 mL), and the filtrate was concentrated. The resulting residue was purified by column chromatography (SiO₂, 30:1 to 1:1 PE/EtOAc to afford methyl 6-((tert-butoxycarbonyl)amino)-4-methoxypyridazine-3-carboxylate (1.20 g, 4.24 mmol, 14% yield) as yellow solid. ¹H NMR: (400 MHz, CDCl₃) δ 8.70-8.30 (m, 1H), 7.90 (s, 1H), 4.01 (s, 3H), 3.99-3.98 (m, 3H), 1.55 (s, 9H).

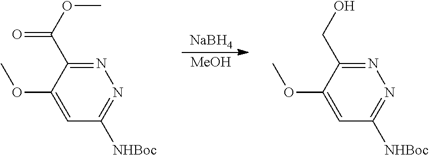 [SMILES: "MeOH" ; COC(=O)c1nnc(NC(=O)OC(C)(C)C)cc1OC ; "NaBH4" ; COc1cc(NC(=O)OC(C)(C)C)nnc1CO]

To a solution of methyl 6-((tert-butoxycarbonyl)amino)-4-methoxypyridazine-3-carboxylate (1.20 g, 4.24 mmol, 1.00 eq) in MeOH (25.0 mL) was added NaBH₄ (1.28 g, 33.8 mmol, 8.00 eq) slowly at 0° C. under N₂. The reaction mixture was stirred at RT for 3 h under N₂. The reaction mixture was treated with saturated aqueous NH4Cl (50.0 mL) at 0° C. and extracted with EtOAc (50.0 mL×2). The combined organic layers were washed with H₂O, dried over Na₂SO₄, filtered, and concentrated under reduced pressure to give a residue. The residue was purified by column chromatography (SiO₂, 10:1 to 1:1 PE/EtOAc to afford tert-butyl (6-(hydroxymethyl)-5-methoxypyridazin-3-yl)carbamate (0.700 g, 2.74 mmol, 65% yield) as a yellow solid. LCMS [M-tBu+H]⁺=200.1 m/z.

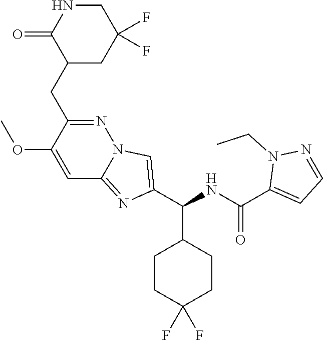 [SMILES: CCn1nccc1C(=O)N[C@H](c1cn2nc(CC3CC(F)(F)CNC3=O)c(OC)cc2n1)C1CCC(F)(F)CC1]

The title compound was synthesized using General Procedure 7, employing tert-butyl (6-(hydroxymethyl)-5-methoxypyridazin-3-yl)carbamate, General Procedure 8, employing 1-(tert-butyl) 3-methyl 5,5-difluoro-2-oxopiperidine-1,3-dicarboxylate and tert-butyl (6-(chloromethyl)-5-methoxypyridazin-3-yl)carbamate, General Procedure 9, employing 1-(tert-butyl) 3-methyl 3-((6-((tert-butoxycarbonyl)amino)-4-methoxypyridazin-3-yl)methyl)-5,5-difluoro-2-oxopiperidine-1,3-dicarboxylate, General Procedure 1, employing methyl 3-((6-amino-4-methoxypyridazin-3-yl)methyl)-5,5-difluoro-2-oxopiperidine-3-carboxylate and benzyl (S)-(3-bromo-1-(4,4-difluorocyclohexyl)-2-oxopropyl)carbamate, General Procedure 3, employing methyl 3-((2-((S)-(((benzyloxy)carbonyl)amino)(4,4-difluorocyclohexyl)methyl)-7-methoxyimidazo[1,2-b]pyridazin-6-yl)methyl)-5,5-difluoro-2-oxopiperidine-3-carboxylate, General Procedure 4, employing 1-ethyl-1H-pyrazole-5-carboxylic acid and methyl 3-((2-((S)-amino(4,4-difluorocyclohexyl)methyl)-7-methoxyimidazo[1,2-b]pyridazin-6-yl)methyl)-5,5-difluoro-2-oxopiperidine-3-carboxylate, General Procedure 5, employing methyl 3-((2-((S)-(4,4-difluorocyclohexyl)(1-ethyl-1H-pyrazole-5-carboxamido)methyl)-7-methoxyimidazo[1,2-b]pyridazin-6-yl)methyl)-5,5-difluoro-2-oxopiperidine-3-carboxylate, and General Procedure 6, employing 3-((2-((S)-(4,4-difluorocyclohexyl)(1-ethyl-1H-pyrazole-5-carboxamido)methyl)-7-methoxyimidazo[1,2-b]pyridazin-6-yl)methyl)-5,5-difluoro-2-oxopiperidine-3-carboxylic acid to afford Compound 75, N-((1S)-(6-((5,5-difluoro-2-oxopiperidin-3-yl)methyl)-7-methoxyimidazo[1,2-b]pyridazin-2-yl)(4,4-difluorocyclohexyl)methyl)-1-ethyl-1H-pyrazole-5-carboxamide, (11.22 mg, 19.6 μmol) as a white solid. LCMS [M+H]⁺=566.3 m/z.

### Example 73: Preparation of Compound 76 and Compound 77

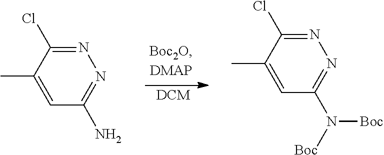

To a solution of 6-chloro-5-methylpyridazin-3-amine (10.0 g, 69.6 mmol, 1.00 eq) in DCM (100 mL) was added Boc₂O (19.7 g, 90.5 mmol, 20.8 mL, 1.30 eq) and DMAP (2.55 g, 20.9 mmol, 0.30 eq). The reaction mixture was stirred at RT for 2 h. The reaction mixture was partitioned between EtOAc (200 mL) and H₂O (200 mL). The organic phase was separated, dried over Na₂SO₄, filtered, and concentrated under reduced pressure to give a residue. The residue was purified by column chromatography (SiO₂, 5:1 to 3:1 PE/EtOAc) to afford tert-butyl (tert-butoxy carbonyl)(6-chloro-5-methylpyridazin-3-yl)carb amate (11.0 g, 31.9 mmol, 46% yield) as a white solid. ¹H NMR: (400 MHz, CDCl₃) δ 7.41 (s, 1H), 2.43 (s 3H), 1.53-1.47 (m, 18H).

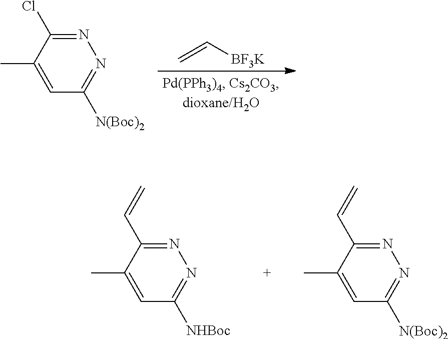

To a solution of tert-butyl (tert-butoxycarbonyl)(6-chloro-5-methylpyridazin-3-yl)carbamate (11.0 g, 31.9 mmol, 1.00 eq) in dioxane (50.0 mL) and H₂O (20.0 mL) was added potassium trifluoro(vinyl)borate (21.4 g, 159 mmol, 5.00 eq), Cs₂CO₃ (31.2 g, 95.9 mmol, 3.00 eq), and Pd(PPh₃)₄ (7.39 g, 6.40 mmol, 0.20 eq). The reaction mixture was stirred at 90° C. for 6 h. The reaction mixture was partitioned between EtOAc (200 mL) and H₂O (200 mL). The organic phase was separated, dried over Na₂SO₄, filtered, and concentrated under reduced pressure to give a residue. The residue was purified by column chromatography (SiO₂, 5:1 to 3:1 PE/EtOAc) to afford tert-butyl (5-methyl-6-vinylpyridazin-3-yl)carbamate (1.20 g, 5.10 mmol, 16% yield) as a white solid (¹H NMR: (400 MHz, CDCl₃) δ 8.02 (s, 1H), 7.73 (s 1H), 7.04-6.82 (m, 1H), 6.50-6.40 (m, 1H), 5.61-5.54 (m, 1H), 2.40-2.27 (m, 3H), 1.57-1.51 (s, 9H)) and tert-butyl (tert-butoxycarbonyl)(5-methyl-6-vinylpyridazin-3-yl)carbamate (1.00 g, 2.98 mmol, 9.3% yield) as a yellow oil (¹H NMR: (400 MHz, CDCl₃) δ 7.31-7.23 (m, 1H), 7.05-6.92 (m 1H), 6.67-6.56 (m, 1H), 5.74-5.66 (m, 1H), 2.47-2.32 (m, 3H), 1.52-1.46 (m, 18H)).

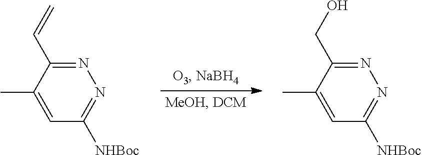 [SMILES: C=Cc1nnc(NC(=O)OC(C)(C)C)cc1C ; Cc1cc(NC(=O)OC(C)(C)C)nnc1CO]

OZONE (15 psi) was bubbled into a solution of tert-butyl (5-methyl-6-vinylpyridazin-3-yl)carbamate (700 mg, 2.98 mmol, 1.00 eq) in DCM (20.0 mL) and MeOH (10.0 mL) at −78° C. for 30 min. Then NaBH₄ (900 mg, 23.8 mmol, 8.00 eq) was added dropwise at 0° C. The resulting mixture was stirred at 0° C. for 1.5 h. The reaction mixture was diluted with H₂O (30.0 mL), and extracted with EtOAc (30 mL×3), dried over anhydrous Na₂SO₄, filtrated, and concentrated under reduced pressure. The residue was purified by prep-TLC (SiO₂, 10:1 DCM:MeOH) to afford tert-butyl (6-(hydroxymethyl)-5-methylpyridazin-3-yl)carb amate (260 mg, 1.09 mmol, 37% yield) as a white solid. LCMS [M+H]⁺=240.0 m/z.

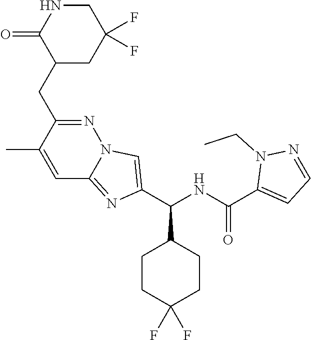 [SMILES: CCn1nccc1C(=O)N[C@H](c1cn2nc(CC3CC(F)(F)CNC3=O)c(C)cc2n1)C1CCC(F)(F)CC1]

The title compound was synthesized using General Procedure 7, employing tert-butyl (6-(hydroxymethyl)-5-methylpyridazin-3-yl)carbamate, General Procedure 8, employing tert-butyl (6-(chloromethyl)-5-methylpyridazin-3-yl)carbamate and 1-(tert-butyl) 3-methyl 5,5-difluoro-2-oxopiperidine-1,3-dicarboxylate, General Procedure 9, employing 1-(tert-butyl) 3-methyl 3-((6-((tert-butoxycarbonyl)amino)-4-methylpyridazin-3-yl)methyl)-5,5-difluoro-2-oxopiperidine-1,3-dicarboxylate, General Procedure 1, employing benzyl (S)-(3-bromo-1-(4,4-difluorocyclohexyl)-2-oxopropyl)carbamate and methyl 3-((6-amino-4-methylpyridazin-3-yl)methyl)-5,5-difluoro-2-oxopiperidine-3-carboxylate (TFA salt), General Procedure 3, employing methyl 3-((2-((S)-(((benzyloxy)carbonyl)amino)(4,4-difluorocyclohexyl)methyl)-7-methylimidazo[1,2-b]pyridazin-6-yl)methyl)-5,5-difluoro-2-oxopiperidine-3-carboxylate, General Procedure 4, employing methyl 3-((2-((S)-amino(4,4-difluorocyclohexyl)methyl)-7-methylimidazo[1,2-b]pyridazin-6-yl)methyl)-5,5-difluoro-2-oxopiperidine-3-carboxylate and 1-ethyl-1H-pyrazole-5-carboxylic acid, General Procedure 5, employing methyl 3-((2-((S)-(4,4-difluorocyclohexyl)(1-ethyl-1H-pyrazole-5-carboxamido)methyl)-7-methylimidazo[1,2-b]pyridazin-6-yl)methyl)-5,5-difluoro-2-oxopiperidine-3-carboxylate, and General Procedure 6, employing 3-((2-((S)-(4,4-difluorocyclohexyl)(1-ethyl-1H-pyrazole-5-carboxamido)methyl)-7-methylimidazo[1,2-b]pyridazin-6-yl)methyl)-5,5-difluoro-2-oxopiperidine-3-carboxylic acid to afford N-((1S)-(6-((5,5-difluoro-2-oxopiperidin-3-yl)methyl)-7-methylimidazo[1,2-b]pyridazin-2-yl)(4,4-difluorocyclohexyl)methyl)-1-ethyl-1H-pyrazole-5-carboxamide, which was purified by chiral SFC (DAICEL CHIRALCEL OD (250 mm×30 mm, 10 μm); 30% mobile phase: [0.1% NH₃H₂O MeOH]) to afford the title compound Compound 76 (24.95 mg, 43.2 μmol) as the first eluting single stereoisomer and white solid. LCMS [M+H]⁺=550.3 m/z. Compound 77 was isolated as the second eluting, single stereoisomer (20.62 mg, 36.2 μmol) as a white solid. LCMS [M+H]⁺=550.3 m/z.

### Example 74: Preparation of Compounds 78 and 79

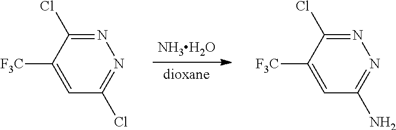 [SMILES: FC(F)(F)c1cc(Cl)nnc1Cl ; "NH3·H2O" ; Nc1cc(C(F)(F)F)c(Cl)nn1]

To a solution of 3,6-dichloro-4-(trifluoromethyl)pyridazine (16.5 g, 76.0 mmol, 1.00 eq) in dioxane (165 mL) was added NH₃·H₂O (205 g, 1.52 μmol, 225 mL, 26.0% purity, 20.0 eq) at RT. The reaction mixture was stirred at 60° C. for 16 h. The reaction mixture was diluted with H₂O (200 mL) and extracted with EtOAc (200 mL×3). The combined organic layers were washed with brine (200 mL), dried over Na₂SO₄, filtered, and concentrated under reduced pressure to give a residue. The residue was purified by flash silica gel chromatography (EtOAc/PE, 0-40%) to afford 6-chloro-5-(trifluoromethyl)pyridazin-3-amine (5.05 g, 25.6 mmol, 34% yield) as a yellow solid. ¹H NMR: (400 MHz, CDCl₃) δ 7.09 (s, 1H), 5.38 (br. s, 2H).

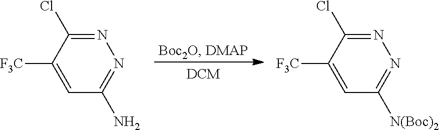

To a solution of 6-chloro-5-(trifluoromethyl)pyridazin-3-amine (5.16 g, 26.1 mmol, 1.00 eq) in DCM (51.6 mL) was added Boc₂O (14.2 g, 65.3 mmol, 15.0 mL, 2.50 eq) and DMAP (3.19 g, 26.1 mmol, 1.00 eq). The reaction mixture was stirred at RT for 12 h. The reaction mixture was diluted with H₂O (50 mL) and extracted with DCM (50 mL×2). The combined organic layers were washed with brine (50 mL), dried over Na₂SO₄, filtered, and concentrated under reduced pressure to afford tert-butyl (tert-butoxycarbonyl)(6-chloro-5-(trifluoromethyl)pyridazin-3-yl)carbamate (13.0 g, crude) as a brown solid.

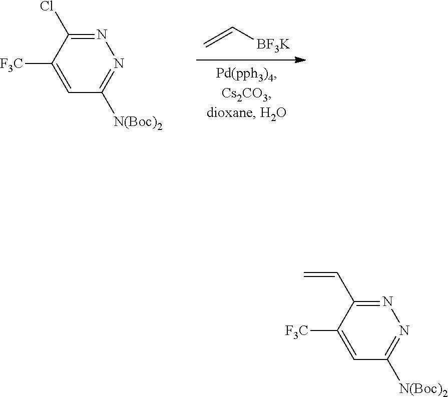

To a solution of tert-butyl (tert-butoxycarbonyl)(6-chloro-5-(trifluoromethyl)pyridazin-3-yl)carbamate (1.30 g, 3.27 mmol, 1.00 eq) in dioxane (36.0 mL) and H₂O (4.00 mL) was added potassium trifluoro(vinyl)borate (482 mg, 3.59 mmol, 1.10 eq) and Cs₂CO₃ (2.13 g, 6.54 mmol, 2.00 eq) at RT. The reaction mixture was degassed and purged with N₂ (3×) and treated with Pd(PPh₃)₄ (378 mg, 327 μmol, 0.100 eq) under N₂. The reaction mixture was stirred at 90° C. for 12 h. The residue was diluted with H₂O (40 mL) and extracted with EtOAc (50 mL×2), the combined organic layers were washed with brine (50 mL), dried over Na₂SO₄, filtered, and concentrated under reduced pressure to give a residue. The residue was purified by silica gel chromatography (EtOAc/PE, 0-10%) to afford tert-butyl (tert-butoxycarbonyl)(5-(trifluoromethyl)-6-vinylpyridazin-3-yl)carbamate (850 mg, 2.18 mmol, 67% yield) as a yellow oil. ¹H NMR. (400 MHz, CDCl₃) δ 7.75 (s, 1H), 7.15-7.02 (m, 1H), 6.85 (d, J=16.8 Hz, 1H), 5.83 (dd, J₁=12.4 Hz, J₂=1.6 Hz, 1H), 1.51 (s, 18H).

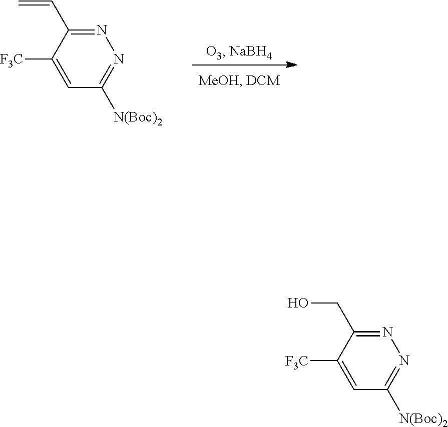

Ozone was bubbled into a solution of tert-butyl (tert-butoxycarbonyl)(5-(trifluoromethyl)-6-vinylpyridazin-3-yl)carbamate (1.30 g, 3.34 mmol, 1.00 eq) in DCM (30.0 mL) and MeOH (3.00 mL) at −70° C. for 30 min. After excess O₃ was purged by N₂, NaBH₄ (410 mg, 10.8 mmol, 3.25 eq) was added at 0° C. The reaction mixture was stirred at 0° C. for 1 h. The reaction mixture was treated with saturated aqueous NH₄Cl (20 mL) at 0° C. and then diluted with H₂O (20 mL) and extracted with DCM (30 mL×2). The combined organic layers were washed with brine, dried over Na₂SO₄, filtered, and concentrated under reduced pressure to give a residue. The residue was purified by silica gel chromatography (EtOAc/PE, 0-30%) to afford tert-butyl (tert-butoxycarbonyl)(6-(hydroxymethyl)-5-(trifluoromethyl)pyridazin-3-yl)carbamate (850 mg, 2.10 mmol, 63% yield) as a yellow oil.

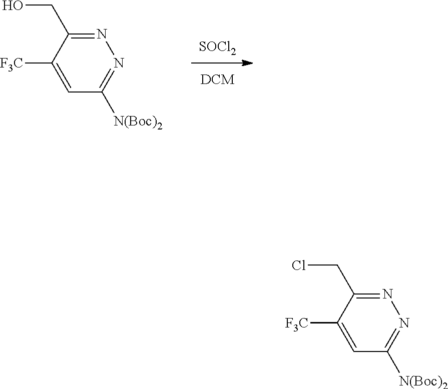

To a solution of tert-butyl (tert-butoxycarbonyl)(6-(hydroxymethyl)-5-(trifluoromethyl)pyridazin-3-yl)carbamate (700 mg, 1.78 mmol, 1.00 eq) in DCM (7.00 mL) was added SOCl₂ (635 mg, 5.34 mmol, 387 μL, 3.00 eq) at 0° C. The reaction mixture was stirred at RT for 1 h. The reaction mixture was concentrated under reduced pressure at 30° C. to afford tert-butyl (tert-butoxycarbonyl)(6-(chloromethyl)-5-(trifluoromethyl)pyridazin-3-yl)carbamate (890 mg, crude) as a red solid.

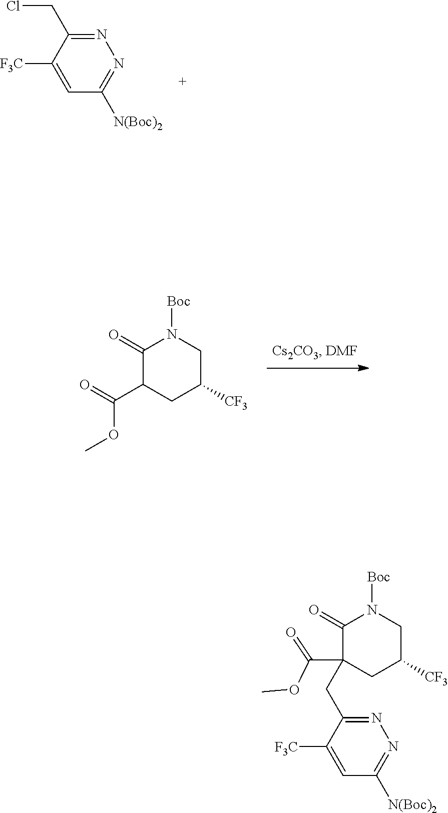

To a solution of 1-(tert-butyl) 3-methyl (5R)-2-oxo-5-(trifluoromethyl)piperidine-1,3-dicarboxylate (844 mg, 2.59 mmol, 1.20 eq) in DMF (4.00 mL) was added Cs₂CO₃ (1.41 g, 4.32 mmol, 2.00 eq) at RT for 30 min. Then tert-butyl (tert-butoxycarbonyl)(6-(chloromethyl)-5-(trifluoromethyl)pyridazin-3-yl)carbamate (890 mg, 2.16 mmol, 1.00 eq) in DMF (2.00 mL) was added at RT. The reaction mixture was stirred at RT for 12 h. The reaction mixture was diluted with H₂O (60 mL) and extracted with EtOAc (30 mL×3). The combined organic layers were washed with brine, dried over Na₂SO₄, filtered, and concentrated under reduced pressure to give a residue. The residue was purified by prep-HPLC (Phenomenex luna C18 250×80 mm×10 μm; eluting with 55-75% ACN with water (0.1% FA)) to afford 1-(tert-butyl) 3-methyl (5R)-3-((6-(bis(tert-butoxycarbonyl)amino)-4-(trifluoromethyl)pyridazin-3-yl)methyl)-2-oxo-5-(trifluoromethyl)piperidine-1,3-dicarboxylate (449 mg, 688 μmol, 32% yield) as a yellow oil. LCMS [M-Boc+H]⁺=601.1 m/z. ¹H NMR (400 MHz, CDCl₃) δ 8.52 (s, 1H), 4.07-3.87 (m, 2H), 3.33 (d, J=7.6 Hz, 3H), 3.74-3.53 (m, 2H), 3.17-2.96 (m, 1H), 2.37-2.20 (m, 2H), 1.54 (d, J=9.6 Hz, 18H).

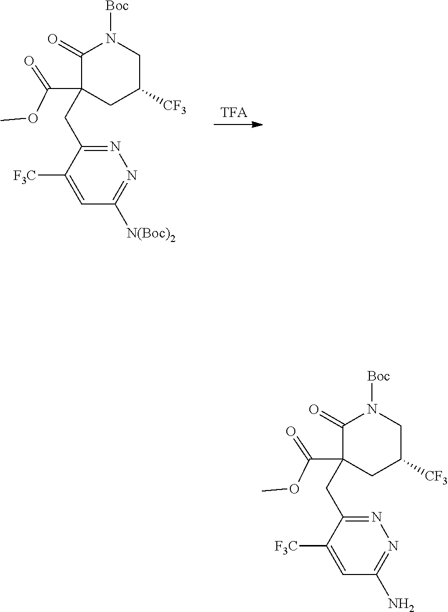

To a solution of 1-(tert-butyl) 3-methyl (5R)-3-((6-(bis(tert-butoxycarbonyl)amino)-4-(trifluoromethyl)pyridazin-3-yl)methyl)-2-oxo-5-(trifluoromethyl)piperidine-1,3-dicarboxylate (449 mg, 748 μmol, 1.00 eq) in DCM (2.00 mL) was added TFA (3.07 g, 26.9 mmol, 1.99 mL, 36.0 eq) at 0° C. The reaction mixture was stirred at RT for 2 h. The reaction mixture was concentrated under reduced pressure to afford 1-(tert-butyl) 3-methyl (5R)-3-((6-amino-4-(trifluoromethyl)pyridazin-3-yl)methyl)-2-oxo-5-(trifluoromethyl)piperidine-1,3-dicarboxylate (384 mg, crude, TFA salt) as a yellow oil. LCMS [M-Boc+H]⁺=401.1 m/z.

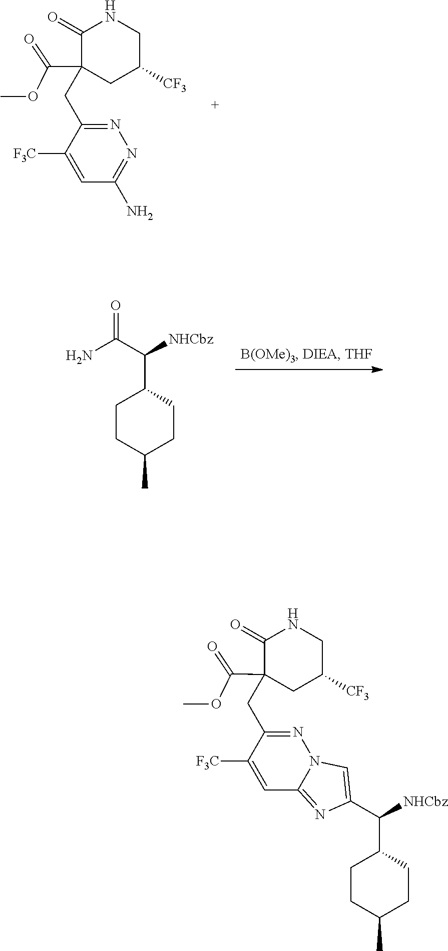 [SMILES: COC(=O)C1(Cc2nnc(N)cc2C(F)(F)F)C[C@@H](C(F)(F)F)CNC1=O ; C[C@H]1CC[C@H]([C@H](NC(=O)OCc2ccccc2)C(N)=O)CC1 ; COC(=O)C1(Cc2nn3cc([C@@H](NC(=O)OCc4ccccc4)[C@H]4CC[C@H](C)CC4)nc3cc2C(F)(F)F)C[C@@H](C(F)(F)F)CNC1=O]

To a solution of 1-(tert-butyl) 3-methyl (5R)-3-((6-amino-4-(trifluoromethyl)pyridazin-3-yl)methyl)-2-oxo-5-(trifluoromethyl)piperidine-1,3-dicarboxylate (90.0 mg, 225 μmol, 1.00 eq) and benzyl ((S)-2-amino-i-((1r,4S)-4-methylcyclohexyl)-2-oxoethyl)carbamate (129 mg, 337 mol, 1.50 eq) in THE (5.00 mL) was added DIEA (174 mg, 1.35 mmol, 235 μL, 6.00 eq) and B(OMe)₃ (140 mg, 1.35 mmol, 152 μL, 6.00 eq). The reaction mixture was stirred at 70° C. for 12 h. The reaction mixture was diluted with H₂O (60 mL) and extracted with EtOAc (30 mL×3). The combined organic layers were washed with brine, dried over Na₂SO₄, filtered, and concentrated under reduced pressure to give a residue. The residue was purified by prep-HPLC (PhenomenexLuna C18 150×25 mm×10 μm; eluting with ACN and water (0.1% FA) 57-87%) to afford methyl (5R)-3-((2-((S)-(((benzyloxy)carbonyl)amino)((1r,4S)-4-methylcyclohexyl)methyl)-7-(trifluoromethyl)imidazo[1,2-b]pyridazin-6-yl)methyl)-2-oxo-5-(trifluoromethyl)piperidine-3-carboxylate (127 mg, 183 μmol, 82% yield) as a yellow solid. LCMS [M+H]⁺=684.3 m/z. ¹H NMR (400 MHz, CDCl₃) δ 8.18 (s, 1H), 7.83-7.72 (m, 1H), 7.36-7.32 (m, 6H), 6.04-5.86 (m, 1H), 5.64-5.44 (m, 1H), 5.11-5.07 (m, 2H), 4.84-4.73 (m, 1H), 3.86-3.80 (m, 3H), 3.61-3.57 (m, 3H), 2.49-2.40 (m, 1H), 1.96-1.86 (m, 2H), 1.79-1.72 (m, 3H), 1.53-1.46 (m, 2H), 1.08-1.00 (m, 3H), 0.95-0.86 (m, 4H).

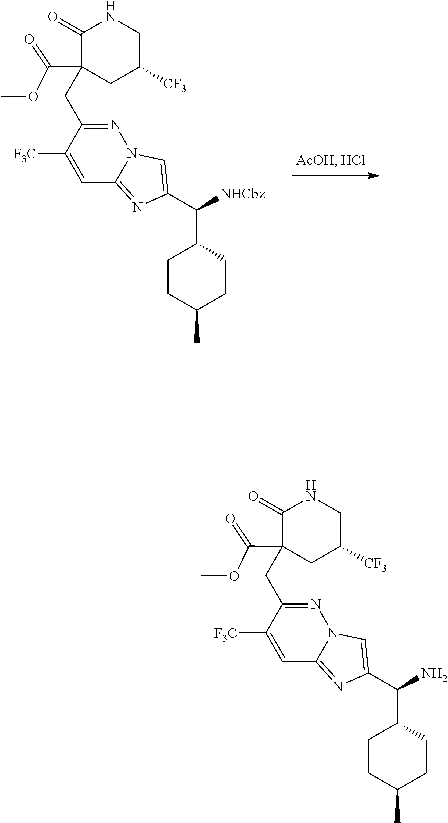 [SMILES: COC(=O)C1(Cc2nn3cc([C@@H](NC(=O)OCc4ccccc4)[C@H]4CC[C@H](C)CC4)nc3cc2C(F)(F)F)C[C@@H](C(F)(F)F)CNC1=O ; COC(=O)C1(Cc2nn3cc([C@@H](N)[C@H]4CC[C@H](C)CC4)nc3cc2C(F)(F)F)C[C@@H](C(F)(F)F)CNC1=O]

To a solution of methyl (5R)-3-((2-((S)-(((benzyloxy)carbonyl)amino)((1r,4S)-4-methylcyclohexyl)methyl)-7-(trifluoromethyl)imidazo[1,2-b]pyridazin-6-yl)methyl)-2-oxo-5-(trifluoromethyl)piperidine-3-carboxylate (161 mg, 236 μmol, 1.00 eq) in AcOH (0.300 mL) was added HCl (12 M, 3.00 mL, 153 eq). The reaction mixture was stirred at 60° C. for 20 min. The reaction mixture was quenched by saturated aqueous Na₂CO₃ (40 mL) and extracted with EtOAc (30 mL×3), dried over Na₂SO₄, filtered, and concentrated under reduced pressure to afford methyl (5R)-3-((2-((S)-amino((1r,4S)-4-methylcyclohexyl)methyl)-7-(trifluoromethyl)imidazo[1,2-b]pyridazin-6-yl)methyl)-2-oxo-5-(trifluoromethyl)piperidine-3-carboxylate (140 mg, crude) as a yellow solid. LCMS [M+H]⁺=550.2 m/z.

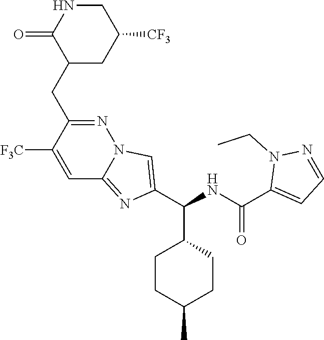 [SMILES: CCn1nccc1C(=O)N[C@H](c1cn2nc(CC3C[C@@H](C(F)(F)F)CNC3=O)c(C(F)(F)F)cc2n1)[C@H]1CC[C@H](C)CC1]

The title compounds were prepared using General Procedure 4, employing methyl (5R)-3-((2-((S)-amino((1r,4S)-4-methylcyclohexyl)methyl)-7-(trifluoromethyl)imidazo[1,2-b]pyridazin-6-yl)methyl)-2-oxo-5-(trifluoromethyl)piperidine-3-carboxylate and 1-ethyl-1H-pyrazole-5-carboxylic acid, General procedure 5, employing methyl (5R)-3-((2-((S)-(1-ethyl-1H-pyrazole-5-carboxamido)((1r,4S)-4-methylcyclohexyl)methyl)-7-(trifluoromethyl)imidazo[1,2-b]pyridazin-6-yl)methyl)-2-oxo-5-(trifluoromethyl)piperidine-3-carboxylate, and General Procedure 6, employing (5R)-3-((2-((S)-(1-ethyl-1H-pyrazole-5-carboxamido)((1r,4S)-4-methylcyclohexyl)methyl)-7-(trifluoromethyl)imidazo[1,2-b]pyridazin-6-yl)methyl)-2-oxo-5-(trifluoromethyl)piperidine-3-carboxylic acid to afford 1-ethyl-N-((1S)-((1r,4S)-4-methylcyclohexyl)(6-(((5R)-2-oxo-5-(trifluoromethyl)piperidin-3-yl)methyl)-7-(trifluoromethyl)imidazo[1,2-b]pyridazin-2-yl)methyl)-1H-pyrazole-5-carboxamide, which was purified by chiral prep-SFC (REGIS (S,S) WHELK-01 (250 mm×25 mm, 10 μm); mobile phase: 40% 0.1% NH₃H₂O IPA]) to afford Compound 78 as the first eluting, single stereoisomer (22.7 mg, 37.0 μmol) as a white solid (LCMS [M=H]⁺=614.2 m z) and Compound 79 as the second eluting, single stereoisomer (16.3 mg, 26.6 μmol) as a yellow solid (LCMS [M=H]⁺=614.2 m z).

### Example 75: Preparation of Compound 80

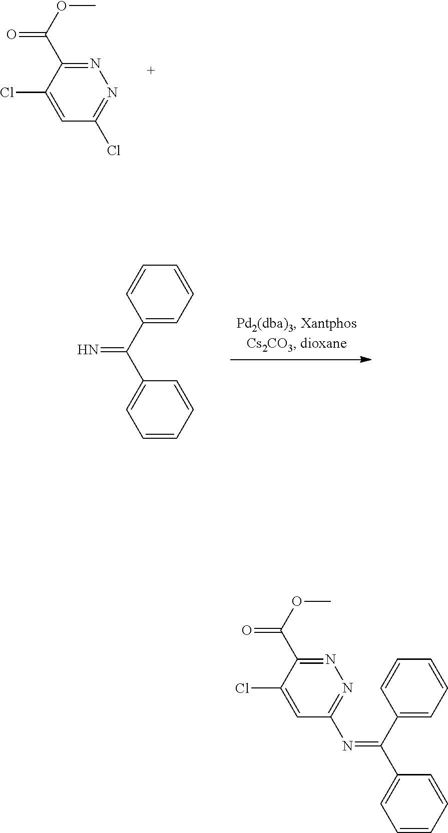 [SMILES: COC(=O)c1nnc(Cl)cc1Cl ; N=C(c1ccccc1)c1ccccc1 ; COC(=O)c1nnc(N=C(c2ccccc2)c2ccccc2)cc1Cl]

To a solution of methyl 4,6-dichloropyridazine-3-carboxylate (60.0 g, 290 mmol, 1.00 eq) in dioxane (1000 mL) was added diphenylmethanimine (52.5 g, 290 mmol, 48.6 mL, 1.00 eq), Cs₂CO₃ (189 g, 580 mmol, 2.00 eq), Pd₂(dba)₃ (13.3 g, 14.5 mmol, 0.0500 eq), and Xantphos (16.8 g, 29.0 mmol, 0.100 eq) at RT. The reaction mixture was stirred at 90° C. for 16 h. The reaction mixture was diluted with DCM (1000 mL), then filtered and concentrated under reduced pressure to give a residue. The residue was purified by column chromatography (SiO₂, 5:1 PE:EtOAc) to afford methyl 4-chloro-6-((diphenylmethylene)amino)pyridazine-3-carboxylate (53.4 g, 152 mmol, 52% yield) as a yellow oil. ¹H NMR (400 MHz, CDCl₃) δ 7.50-7.39 (m, 10H), 6.98 (s, 1H), 4.03 (s, 3H).

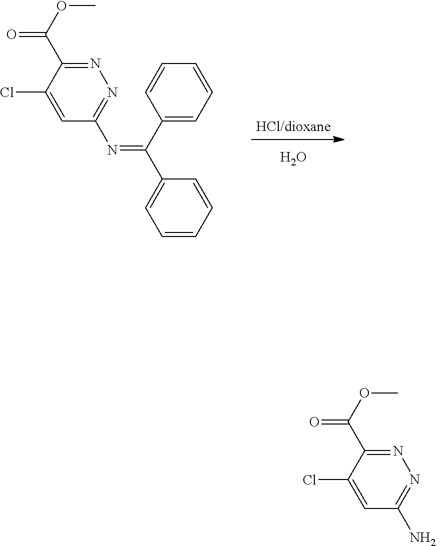

To a solution of methyl 4-chloro-6-((diphenylmethylene)amino)pyridazine-3-carboxylate (53.4 g, 152 mmol, 1.00 eq) in HCl/dioxane (4 M, 534 mL, 14.1 eq) was added H₂O (125 mL) at 0° C. The reaction mixture was stirred at RT for 12 h. The reaction mixture was treated with saturated aqueous NaHCO₃ until the pH was ˜8, then diluted with EtOAc (200 mL) and filtered. The filtrate was extracted with EtOAc (500 mL×3). The filter cake was stirred with 150 mL of 10:1 EtOAc: MeOH, and then filtered. The combined organic layers were dried over anhydrous Na₂SO₄, filtered, and concentrated under reduced pressure to give a residue. The residue was purified by column chromatography (SiO₂, 1:1 PE:EtOAc) to afford methyl 6-amino-4-chloropyridazine-3-carboxylate (13.3 g, 70.9 mmol, 47% yield) as a white solid. LCMS [M=H]⁺=188.0 m/z. ¹H NMR (400 MHz, DMSO-d₆) δ 7.32 (s, 2H), 6.90 (s, 1H), 3.86 (s, 3H).

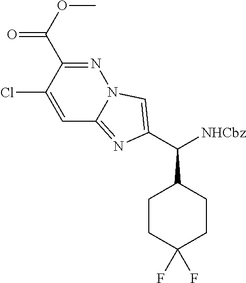 [SMILES: COC(=O)c1nn2cc([C@@H](NC(=O)OCc3ccccc3)C3CCC(F)(F)CC3)nc2cc1Cl]

Methyl (S)-2-((((benzyloxy)carbonyl)amino)(4,4-difluorocyclohexyl)methyl)-7-chloroimidazo[1,2-b]pyridazine-6-carboxylate was prepared according to General Procedure 1, employing methyl 6-amino-4-chloropyridazine-3-carboxylate and benzyl (S)-(3-bromo-1-(4,4-difluorocyclohexyl)-2-oxopropyl)carbamate to afford the desired compound (1.40 g, 2.84 mmol) as a yellow oil.

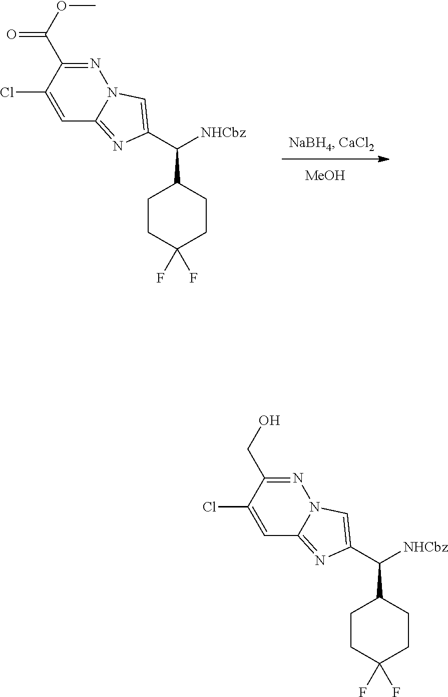

To a solution of methyl (S)-2-((((benzyloxy)carbonyl)amino)(4,4-difluorocyclohexyl)methyl)-7-chloroimidazo[1,2-b]pyridazine-6-carboxylate (1.40 g, 2.84 mmol, 1.00 eq) in MeOH (20.0 mL) was added CaCl₂ (946 mg, 8.52 mmol, 3.00 eq) and NaBH₄ (537 mg, 14.2 mmol, 5.00 eq) at 0° C. The reaction mixture was stirred at RT for 50 min. The reaction mixture was treated with saturated aqueous NH₄Cl (30.0 mL), then concentrated under reduced pressure to remove MeOH. The aqueous layer was extracted with EtOAc (30.0 mL×3). The combined organic layers were dried over anhydrous Na₂SO₄, filtered, and concentrated under reduced pressure to afford benzyl (S)-((7-chloro-6-(hydroxymethyl)imidazo[1,2-b]pyridazin-2-yl)(4,4-difluorocyclohexyl)methyl)carbamate (1.18 g, 2.54 mmol, 89.4% yield) as yellow foam, which was used directly in the next reaction. LCMS [M=H]⁺=465.2 m/z.

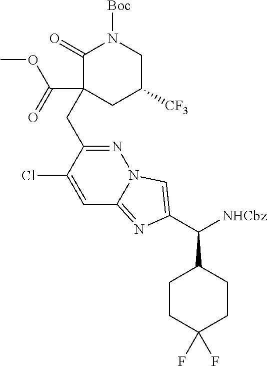 [SMILES: COC(=O)C1(Cc2nn3cc([C@@H](NC(=O)OCc4ccccc4)C4CCC(F)(F)CC4)nc3cc2Cl)C[C@@H](C(F)(F)F)CN(C(=O)OC(C)(C)C)C1=O]

The title compound was prepared using General Procedure 7, employing benzyl (S)-((7-chloro-6-(hydroxymethyl)imidazo[1,2-b]pyridazin-2-yl)(4,4-difluorocyclohexyl)methyl)carbamate, and General Procedure 8, employing benzyl (S)-((7-chloro-6-(chloromethyl)imidazo[1,2-b]pyridazin-2-yl)(4,4-difluorocyclohexyl)methyl)carbamate and 1-(tert-butyl) 3-methyl (5R)-2-oxo-5-(trifluoromethyl)piperidine-1,3-dicarboxylate.

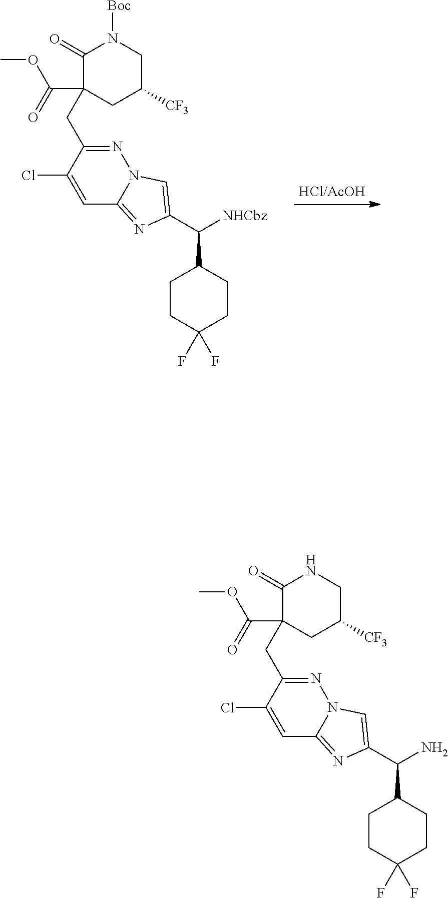

1-(tert-butyl) 3-methyl (5R)-3-((2-((S)-(((benzyloxy)carbonyl)amino)(4,4-difluorocyclohexyl)methyl)-7-chloroimidazo[1,2-b]pyridazin-6-yl)methyl)-2-oxo-5-(trifluoromethyl)piperidine-1,3-dicarboxylate (500 mg, 648 μmol, 1.00 eq) was dissolved in HCl (12 M, 5.00 mL, 92.7 eq) and AcOH (524 mg, 8.73 mmol, 0.500 mL, 13.5 eq) at RT. The reaction mixture was stirred at 60° C. for 1 h. The reaction mixture was diluted with saturated aqueous NaHCO₃ until the pH was ˜8, then extracted with EtOAc (50.0 mL×3). The combined organic layers were dried over anhydrous Na₂SO₄, filtered, and concentrated under reduced pressure to afford methyl (5R)-3-((2-((S)-amino(4,4-difluorocyclohexyl)methyl)-7-chloroimidazo[1,2-b]pyridazin-6-yl)methyl)-2-oxo-5-(trifluoromethyl)piperidine-3-carboxylate (200 mg, 372 μmol, 57% yield) as a yellow gum. LCMS [M=H]⁺=538.2 m/z.

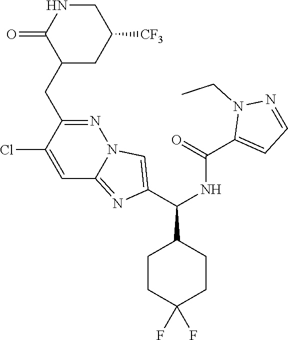 [SMILES: CCn1nccc1C(=O)N[C@H](c1cn2nc(CC3C[C@@H](C(F)(F)F)CNC3=O)c(Cl)cc2n1)C1CCC(F)(F)CC1]

The title compound was prepared according to General Procedure 4, employing 1-ethyl-1H-pyrazole-5-carboxylic acid and methyl (5R)-3-((2-((S)-amino(4,4-difluorocyclohexyl)methyl)-7-chloroimidazo[1,2-b]pyridazin-6-yl)methyl)-2-oxo-5-(trifluoromethyl)piperidine-3-carboxylate, General Procedure 5, employing methyl (5R)-3-((7-chloro-2-((S)-(4,4-difluorocyclohexyl)(1-ethyl-1H-pyrazole-5-carboxamido)methyl)imidazo[1,2-b]pyridazin-6-yl)methyl)-2-oxo-5-(trifluoromethyl)piperidine-3-carboxylate, and General Procedure 6, employing (5R)-3-((7-chloro-2-((S)-(4,4-difluorocyclohexyl)(1-ethyl-1H-pyrazole-5-carboxamido)methyl)imidazo[1,2-b]pyridazin-6-yl)methyl)-2-oxo-5-(trifluoromethyl)piperidine-3-carboxylic acid followed by chiral prep-SFC (REGIS (R,R) WHELK-O1 (250 mm×25 mm, 10 μm); 50% mobile phase: [0.1% NH₃H₂O EtOH]) to afford Compound 80, N-((1S)-(7-chloro-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide, (30.4 mg, 49.8 μmol) as the first eluting, single stereoisomer and a white foam. LCMS [M=H]⁺=602.1 m/z.

### Example 76: Preparation of Compound 81

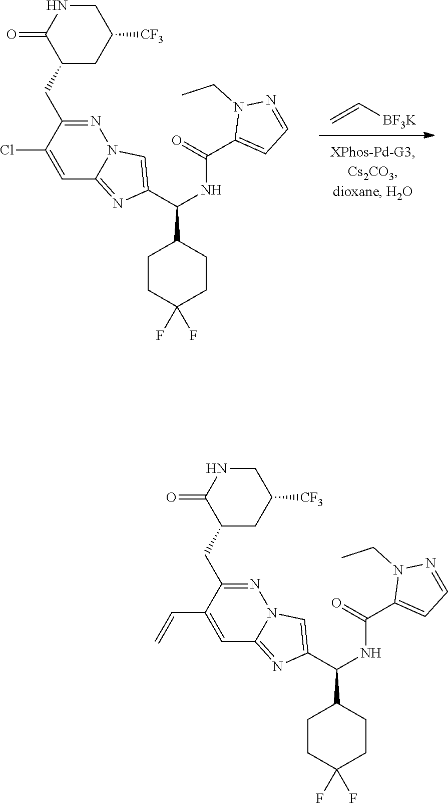

A mixture of N—((S)-(7-chloro-6-(((3R,5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (200 mg, 332 μmol, 1.00 eq), potassium vinyl trifluoroborate (222 mg, 1.66 mmol, 5.00 eq), Cs₂CO₃ (324 mg, 996 μmol, 3.00 eq), and XPhos Pd G3 (28.1 mg, 33.2 μmol, 0.100 eq) in dioxane (4.00 mL) and H₂O (1.00 mL) was degassed and purged with N₂ 3 times, and then the reaction mixture was stirred at 90° C. for 2 h under N₂ atmosphere. The reaction mixture was partitioned between DCM (60.0 mL) and H₂O (60.0 mL). The organic phase was separated, dried over Na₂SO₄, filtered, and concentrated under reduced pressure to give a residue, which was purified by prep-TLC (SiO₂, 10:1 DCM:MeOH) to afford N—((S)-(4,4-difluorocyclohexyl)(6-(((3R,5R)-2-oxo-5-(trifluoromethyl)piperidin-3-yl)methyl)-7-vinylimidazo[1,2-b]pyridazin-2-yl)methyl)-1-ethyl-1H-pyrazole-5-carboxamide (120 mg, 202 μmol, 61% yield) as a white solid. LCMS [M+H]⁺=594.6 m/z.

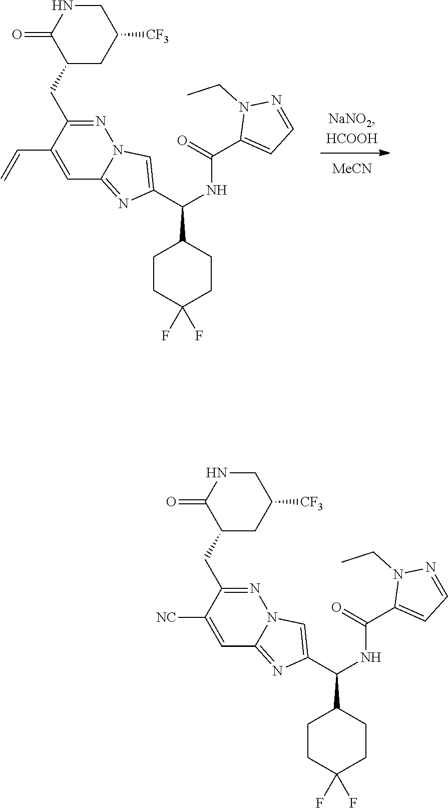 [SMILES: "MeCN" ; C=Cc1cc2nc([C@@H](NC(=O)c3ccnn3CC)C3CCC(F)(F)CC3)cn2nc1C[C@H]1C[C@@H](C(F)(F)F)CNC1=O ; CCn1nccc1C(=O)N[C@H](c1cn2nc(C[C@H]3C[C@@H](C(F)(F)F)CNC3=O)c(C#N)cc2n1)C1CCC(F)(F)CC1]

To a solution of N—((S)-(4,4-difluorocyclohexyl)(6-(((3R,5R)-2-oxo-5-(trifluoromethyl)piperidin-3-yl)methyl)-7-vinylimidazo[1,2-b]pyridazin-2-yl)methyl)-1-ethyl-1H-pyrazole-5-carboxamide (100 mg, 168 μmol, 1.00 eq) and NaNO₂ (58.1 mg, 842.3 μmol, 5.00 eq) in MeCN (1.00 mL) was added HCOOH (202 mg, 4.21 mmol, 25.0 eq). The reaction mixture was stirred at 85° C. for 12 h. The reaction mixture was concentrated under reduced pressure, and the residue was diluted with H₂O (60.0 mL), extracted with DCM (60.0 mL), dried over Na₂SO₄, filtered, and concentrated under reduced pressure to give a residue. The residue was purified by prep-TLC (SiO₂, 10:1 DCM:MeOH) followed by prep-HPLC (Waters Xbridge 150×25 mm×5 um; mobilephase: [water(ammonia hydroxide) and 32-62% ACN]) to afford Compound 81 (4.59 mg, 6.60 μmol, 3.9% yield) as a yellow solid. LCMS [M+H]⁺=593.4 m/z.

### Example 77: Preparation of Compounds 82 and 83

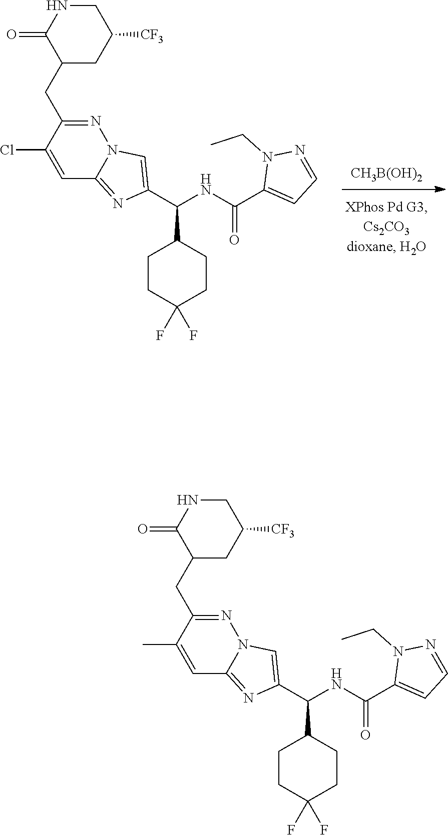

To reaction vial containing N-((1S)-(7-chloro-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (70.0 mg, 116 μmol, 1.00 eq) and methyl boronic acid (174 mg, 2.91 mmol, 25.0 eq) was added Cs₂CO₃ (113 mg, 348 μmol, 3.00 eq), XPhos Pd G3 (9.84 mg, 11.6 μmol, 0.100 eq), H₂O (1.00 mL), and dioxane (8.00 mL). The reaction mixture was stirred at 100° C. for 6 h. The reaction mixture was diluted with H₂O (10.0 mL) and extracted with DCM (30.0 mL×2). The combined organic layers were dried over anhydrous Na₂SO₄, filtered, and concentrated under reduced pressure to give a residue. The residue was purified by prep-TLC. (10:1 DCM:MeOH) to afford N-((1S)-(4,4-difluorocyclohexyl)(7-methyl-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (30.0 mg, 51.5 μmol, 44% yield) as a white solid. The isolated material was further purified by chiral prep-SFC (DAICEL CHIRALPAK IC (250 mm×30 mm, 10 μm); 40% mobile phase: [iPrOH (0.1% NH₃H₂O)]) to afford Compound 82 as the first eluting, single stereoisomer and white solid (2.99 mg, 4.87 μmol, 9.4% yield, LCMS [M+H]⁺=582.3 m z) and Compound 83 as the second eluting single stereoisomer and white solid (10.84 mg, 17.9 μmol, LCMS [M+H]⁺=582.3 m z).

### Example 78: Preparation of Compound 84

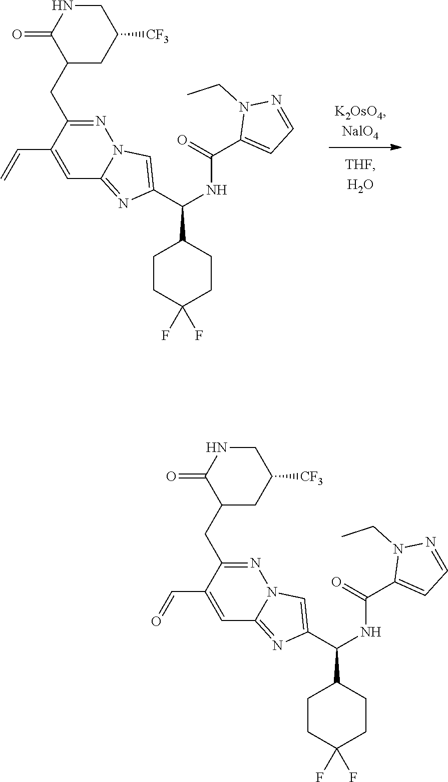 [SMILES: C=Cc1cc2nc([C@@H](NC(=O)c3ccnn3CC)C3CCC(F)(F)CC3)cn2nc1CC1C[C@@H](C(F)(F)F)CNC1=O ; CCn1nccc1C(=O)N[C@H](c1cn2nc(CC3C[C@@H](C(F)(F)F)CNC3=O)c(C=O)cc2n1)C1CCC(F)(F)CC1]

A solution of N-((1S)-(4,4-difluorocyclohexyl)(6-(((5R)-2-oxo-5-(trifluoromethyl)piperidin-3-yl)methyl)-7-vinylimidazo[1,2-b]pyridazin-2-yl)methyl)-1-ethyl-1H-pyrazole-5-carboxamide (160 mg, 269 μmol, 1.00 eq) in THE (2.00 mL) and H₂O (4.00 mL) was added K₂OsO₄-2H₂O (19.8 mg, 53.9 μmol, 0.200 eq) and NaIO₄ (230 mg, 1.08 mmol, 59.7 L, 4.00 eq). The reaction mixture was stirred at RT for 2 h. The reaction mixture was diluted with H₂O (30.0 mL), extracted with EtOAc (30.0 mL×3), dried over anhydrous Na₂SO₄, filtrated, and concentrated under reduced pressure to afford N-((1S)-(4,4-difluorocyclohexyl)(7-formyl-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (150 mg, 251 μmol, quantitative) as a yellow solid. LCMS [M+H]⁺=596.4 m/z.

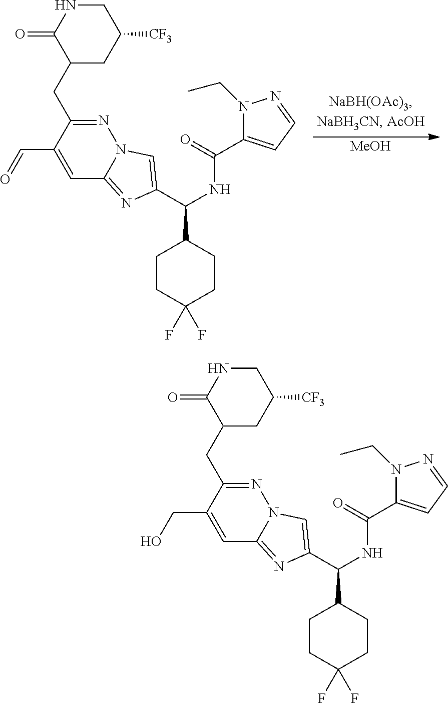

To a solution of N-((1S)-(4,4-difluorocyclohexyl)(7-formyl-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (150 mg, 251 μmol, 1.00 eq) in MeOH (2.00 mL) was added NaBH(OAc)₃ (160 mg, 755 μmol, 3.00 eq) and NaBH₃CN (15.8 mg, 251 μmol, 1.00 eq). The reaction mixture was stirred at RT for 1 h. The reaction mixture was diluted with H₂O (30.0 mL), extracted with EtOAc (30.0 mL×3), dried over anhydrous Na₂SO₄, filtrated, and concentrated under reduced pressure. The residue was purified by prep-TLC(SiO₂, 10:1 DCM:MeOH) to afford N-((1S)-(4,4-difluorocyclohexyl)(7-(hydroxymethyl)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (30.0 mg, 50.2 μmol, 20% yield) as a white solid. LCMS [M+H]⁺=598.4 m/z.

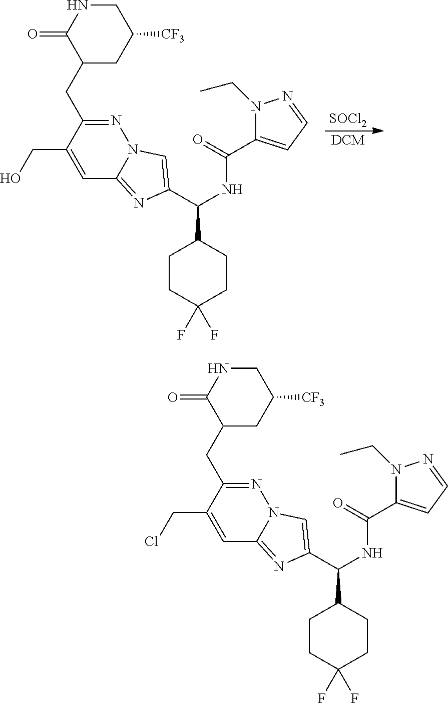

To a solution of N-((1S)-(4,4-difluorocyclohexyl)(7-(hydroxymethyl)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (30.0 mg, 50.2 μmol, 1.00 eq) in DCM (1.00 mL) was added SOCl₂ (5.97 mg, 50.2 μmol, 3.65 μL, 1.00 eq) at 0° C. The reaction mixture was stirred at RT for 30 min. The reaction was concentrated under reduced pressure to afford N-((1S)-(7-(chloromethyl)-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (30.0 mg, 48.7 μmol, 97% yield) as a white solid. LCMS [M+H]⁺=616.3 m/z.

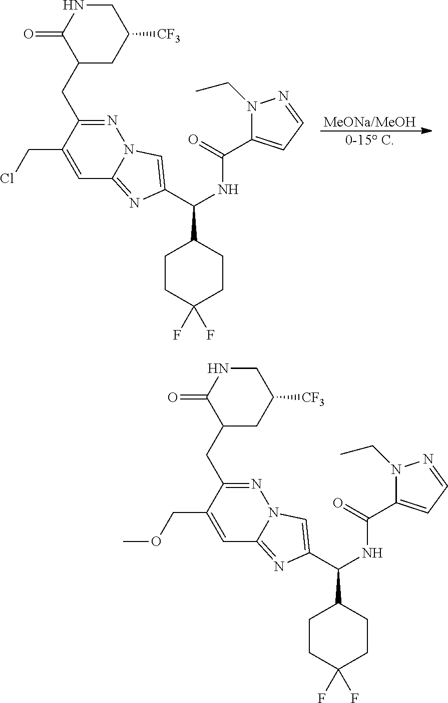

To a solution of N-((1S)-(7-(chloromethyl)-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (30.0 mg, 48.7 μmol, 1.00 eq) in THF (0.500 mL) was added NaOMe (10.0 M, 14.6 μL, 3.00 eq) at 0° C. The reaction mixture was stirred at RT for 30 min. The reaction mixture was diluted with H₂O (30.0 mL), extracted with EtOAc (30.0 mL×3), dried over anhydrous Na₂SO₄, filtrated, and concentrated under reduced pressure. The residue purified by prep-TLC (SiO₂, 10:1 DCM/MeOH) to afford Compound 84, N-((1S)-(4,4-difluorocyclohexyl)(7-(methoxymethyl)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, (8.96 mg 13.42 μmol, 28% yield) as the major stereoisomer and a yellow solid. LCMS [M+H]⁺=612.4 m z.

### Example 79: Preparation of Compounds 85 and 86

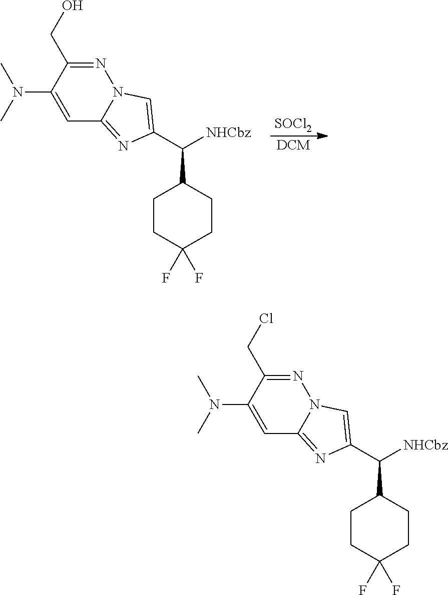

To a solution of benzyl (S)-((4,4-difluorocyclohexyl)(7-(dimethylamino)-6-(hydroxymethyl)imidazo[1,2-b]pyridazin-2-yl)methyl)carbamate (1.00 g, 2.11 mmol, 1.00 eq) in DCM (3.00 mL) was added SOCl₂ (753 mg, 6.34 mmol, 460 μL, 3.00 eq) at 0° C. The reaction mixture was stirred at RT for 1 h. The reaction mixture was concentrated under reduced pressure to afford benzyl (S)-((6-(chloromethyl)-7-(dimethylamino)imidazo[1,2-b]pyridazin-2-yl)(4,4-difluorocyclohexyl)methyl)carbamate (1.00 g, 2.03 mmol, 96% yield) as a yellow solid. LCMS [M+H]⁺=492.3 m/z.

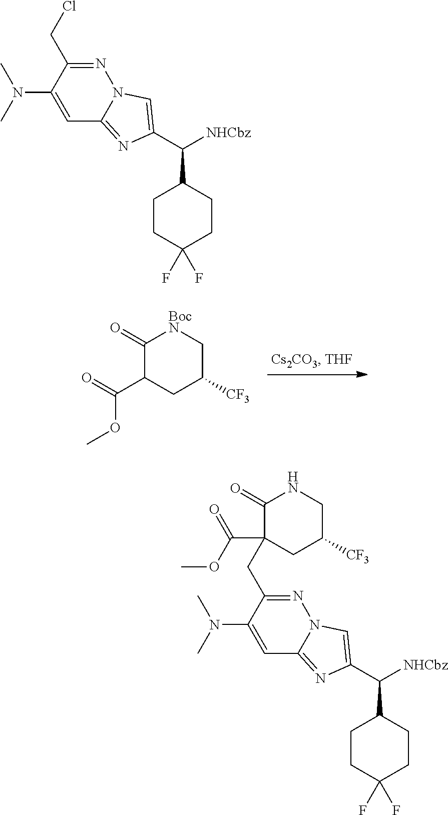 [SMILES: CN(C)c1cc2nc([C@@H](NC(=O)OCc3ccccc3)C3CCC(F)(F)CC3)cn2nc1CCl ; COC(=O)C1C[C@@H](C(F)(F)F)CN(C(=O)OC(C)(C)C)C1=O ; COC(=O)C1(Cc2nn3cc([C@@H](NC(=O)OCc4ccccc4)C4CCC(F)(F)CC4)nc3cc2N(C)C)C[C@@H](C(F)(F)F)CNC1=O]

A solution of 1-(tert-butyl) 3-methyl (5R)-2-oxo-5-(trifluoromethyl)piperidine-1,3-dicarboxylate (915 mg, 4.07 mmol, 2.00 eq) in THF (10.0 mL) was treated with Cs₂CO₃ (1.99 g 6.10 mmol, 3.00 eq), and the reaction mixture was stirred at RT for 30 min before adding benzyl (S)-((6-(chloromethyl)-7-(dimethylamino)imidazo[1,2-b]pyridazin-2-yl)(4,4-difluorocyclohexyl)methyl)carbamate (1.00 g, 2.03 mmol, 1.00 eq). The reaction mixture was stirred at 70° C. for 3.5 h. The reaction mixture was diluted with water and extracted with DCM, dried over Na₂SO₄, filtered, and concentrated under reduced pressure to give a residue. The residue was purified by column chromatography (SiO₂, DCM followed by 10:1 PE:EtOAc) to afford methyl (5R)-3-((2-((S)-(((benzyloxy)carbonyl)amino)(4,4-difluorocyclohexyl)methyl)-7-(dimethylamino)imidazo[1,2-b]pyridazin-6-yl)methyl)-2-oxo-5-(trifluoromethyl)piperidine-3-carboxylate (650 mg, 954 μmol, 47% yield) as a yellow solid. LCMS [M+H]⁺=681.4 m/z.

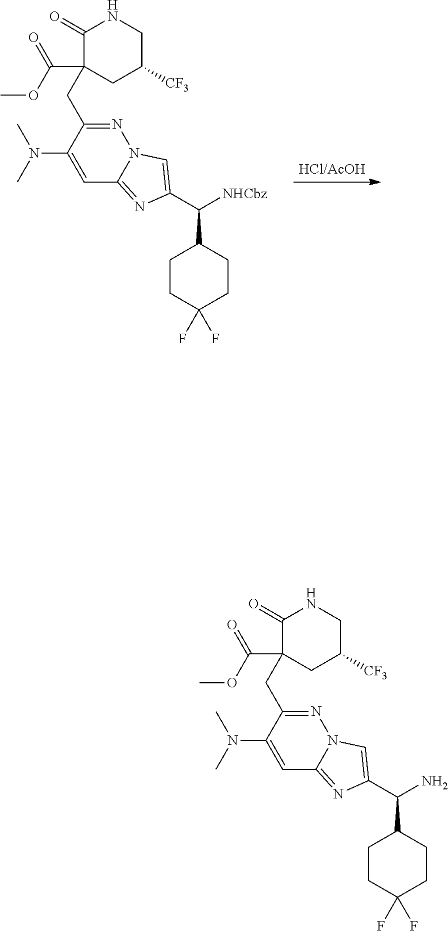

A solution of methyl (5R)-3-((2-((S)-(((benzyloxy)carbonyl)amino)(4,4-difluorocyclohexyl)methyl)-7-(dimethylamino)imidazo[1,2-b]pyridazin-6-yl)methyl)-2-oxo-5-(trifluoromethyl)piperidine-3-carboxylate (650 mg, 954 μmol, 1.00 eq) in HCl (5.00 mL) and AcOH (0.500 mL) was stirred at 50° C. for 30 min. The reaction mixture was adjusted with NaHCO₃ to pH ˜8, and then extracted with DCM (20.0 mL×3). The combined organic layers were dried over anhydrous Na₂SO₄, filtered, and concentrated under reduced pressure to afford methyl (5R)-3-((2-((S)-amino(4,4-difluorocyclohexyl)methyl)-7-(dimethylamino)imidazo[1,2-b]pyridazin-6-yl)methyl)-2-oxo-5-(trifluoromethyl)piperidine-3-carboxylate (40 mg, 731 μmol, 77% yield) as a yellow solid. LCMS [M+H]⁺=547.3 m/z.

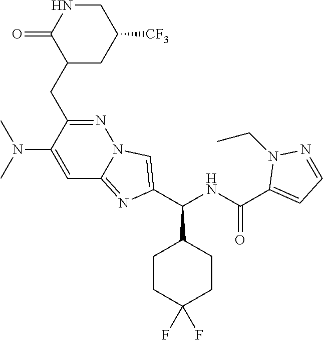

The title compound was prepared according to General Procedure 4, employing methyl (5R)-3-((2-((S)-amino(4,4-difluorocyclohexyl)methyl)-7-(dimethylamino)imidazo[1,2-b]pyridazin-6-yl)methyl)-2-oxo-5-(trifluoromethyl)piperidine-3-carboxylate and 1-ethyl-1H-pyrazole-5-carboxylic acid, General Procedure 5, employing methyl (5R)-3-((2-((S)-(4,4-difluorocyclohexyl)(1-ethyl-1H-pyrazole-5-carboxamido)methyl)-7-(dimethylamino)imidazo[1,2-b]pyridazin-6-yl)methyl)-2-oxo-5-(trifluoromethyl)piperidine-3-carboxylate, and General Procedure 6, employing (5R)-3-((2-((S)-(4,4-difluorocyclohexyl)(1-ethyl-1H-pyrazole-5-carboxamido)methyl)-7-(dimethylamino)imidazo[1,2-b]pyridazin-6-yl)methyl)-2-oxo-5-(trifluoromethyl)piperidine-3-carboxylic acid to afford N-((1S)-(4,4-difluorocyclohexyl)(7-(dimethylamino)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, which was further purified by chiral prep-SFC (DAICEL CHIRALPAK AS (250 mm×30 mm, 10 μm); 30% mobile phase: [ACN/EtOH (0.1% NH₃H₂O)]) to provide Compound 86 as the first eluting, single stereoisomer and orange solid (50.63 mg, 82.42 μmol, LCMS [M+H]⁺=611.3 m z) and Compound 85 as the second eluting, single stereoisomer and orange solid (35.0 mg, 56.75 μmol, LCMS [M+H]⁺=611.3 m/z).

### Example 80: Preparation of Compounds 86 and 87

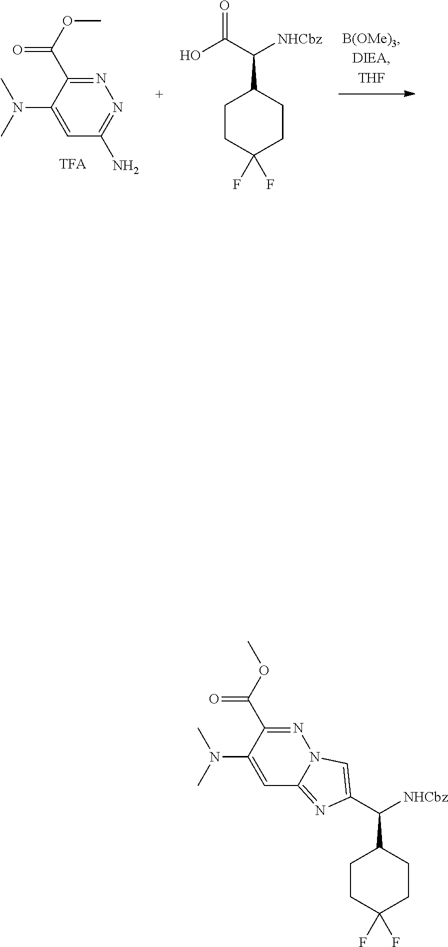

methyl (S)-2-((((benzyloxy)carbonyl)amino)(4,4-difluorocyclohexyl)methyl)-7-(dimethylamino)imidazo[1,2-b]pyridazine-6-carboxylate was synthesized according to General Procedure 1, employing methyl 6-amino-4-(dimethylamino)pyridazine-3-carboxylate and (S)-2-(((benzyloxy)carbonyl)amino)-2-(4,4-difluorocyclohexyl)acetic acid.

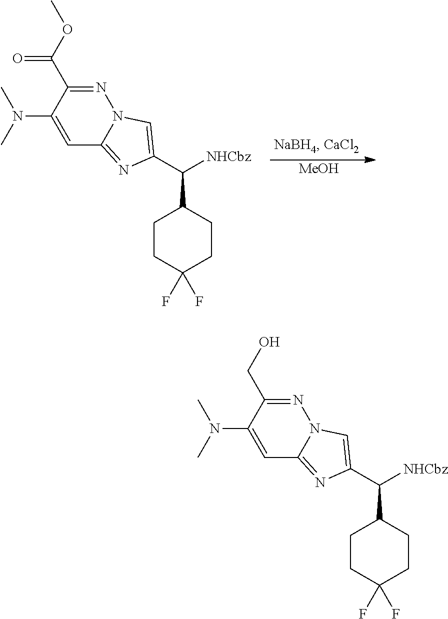 [SMILES: "MeOH" ; COC(=O)c1nn2cc([C@@H](NC(=O)OCc3ccccc3)C3CCC(F)(F)CC3)nc2cc1N(C)C ; CN(C)c1cc2nc([C@@H](NC(=O)OCc3ccccc3)C3CCC(F)(F)CC3)cn2nc1CO]

To a solution of benzyl (S)-((4,4-difluorocyclohexyl)(7-(dimethylamino)-6-(hydroxymethyl)imidazo[1,2-b]pyridazin-2-yl)methyl)carbamate (2.54 g, 5.05 mmol, 1.00 eq) in MeOH (20.0 mL) was added CaCl₂ (1.12 g, 10.11 mmol, 2.00 eq) and NaBH₄ (956 mg, 25.2 mmol, 5.00 eq) at 0° C. The reaction mixture was stirred at RT for 2 h. The reaction mixture was quenched by additions of saturated aqueous NH₄Cl 50 mL at 0° C., and then diluted with H₂O and extracted with EtOAc. The combined organic layers were washed with brine, dried over anhydrous Na₂SO₄, filtered, and concentrated under reduced pressure to afford methyl (S)-2-((((benzyloxy)carbonyl)amino)(4,4-difluorocyclohexyl)methyl)-7-(dimethylamino)imidazo[1,2-b]pyridazine-6-carboxylate (2.00 g, 4.22 mmol, 84% yield) as a yellow solid.

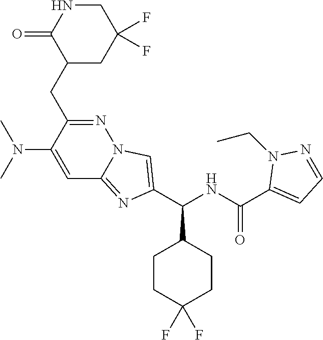

The title compound was prepared according to General Procedure 7, employing methyl (S)-2-((((benzyloxy)carbonyl)amino)(4,4-difluorocyclohexyl)methyl)-7-(dimethylamino)imidazo[1,2-b]pyridazine-6-carboxylate, General Procedure 14, employing benzyl (S)-((6-(chloromethyl)-7-(dimethylamino)imidazo[1,2-b]pyridazin-2-yl)(4,4-difluorocyclohexyl)methyl)carbamate and methyl 5,5-difluoro-2-oxopiperidine-3-carboxylate, General Procedure 15, employing methyl 3-((2-((S)-(((benzyloxy)carbonyl)amino)(4,4-difluorocyclohexyl)methyl)-7-(dimethylamino)imidazo[1,2-b]pyridazin-6-yl)methyl)-5,5-difluoro-2-oxopiperidine-3-carboxylate, General Procedure 4, employing methyl 3-((2-((S)-amino(4,4-difluorocyclohexyl)methyl)-7-(dimethylamino)imidazo[1,2-b]pyridazin-6-yl)methyl)-5,5-difluoro-2-oxopiperidine-3-carboxylate and 1-ethyl-1H-pyrazole-5-carboxylic acid, General Procedure 5, employing methyl 3-((2-((S)-(4,4-difluorocyclohexyl)(1-ethyl-1H-pyrazole-5-carboxamido)methyl)-7-(dimethylamino)imidazo[1,2-b]pyridazin-6-yl)methyl)-5,5-difluoro-2-oxopiperidine-3-carboxylate, General Procedure 6, employing 3-((2-((S)-(4,4-difluorocyclohexyl)(1-ethyl-1H-pyrazole-5-carboxamido)methyl)-7-(dimethylamino)imidazo[1,2-b]pyridazin-6-yl)methyl)-5,5-difluoro-2-oxopiperidine-3-carboxylic acid, to afford N-((1S)-(6-((5,5-difluoro-2-oxopiperidin-3-yl)methyl)-7-(dimethylamino)imidazo[1,2-b]pyridazin-2-yl)(4,4-difluorocyclohexyl)methyl)-1-ethyl-1H-pyrazole-5-carboxamide. This compound was further purified by chiral prep-SFC to afford Compound 86 as the first eluting, single stereoisomer (12.03 mg, 19.59 μmol, LCMS [M+H]⁺=579.4 m z) as a white solid and Compound 87 as the second eluting, single stereoisomer (14.44 mg, 23.72 μmol, LCMS [M+H]⁺=579.4 m z) as a white solid.

### Example 81: Preparation of Compounds 88 and 89

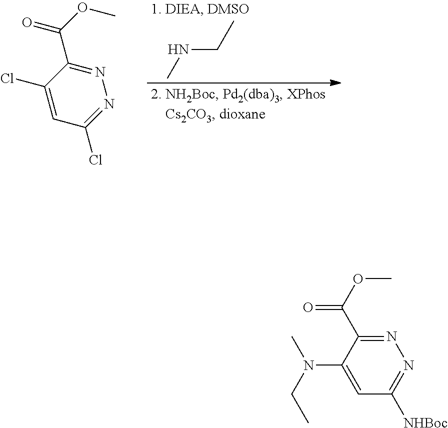

Methyl 6-((tert-butoxycarbonyl)amino)-4-(ethyl(methyl)amino)pyridazine-3-carboxylate was prepared according to General Procedure 10, employing N-methylethanamine, and General Procedure 11, employing methyl 6-chloro-4-(ethyl(methyl)amino)pyridazine-3-carboxylate.

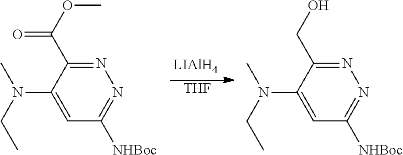 [SMILES: CCN(C)c1cc(NC(=O)OC(C)(C)C)nnc1C(=O)OC ; CCN(C)c1cc(NC(=O)OC(C)(C)C)nnc1CO ; "THF"]

To a solution of Methyl 6-((tert-butoxycarbonyl)amino)-4-(ethyl(methyl)amino)pyridazine-3-carboxylate (4.00 g, 12.9 mmol, 1.00 eq) in 70 mL of THF was added LiAlH₄ (2.5 M, 5.16 mL, 1.00 eq) at 0° C. The reaction mixture was stirred at RT for 30 min before diluting with 10 mL of water and extracting with EtOAc (3×10 mL). The combined organic layers were dried over Na2SO4, filtered, and concentrated under reduced pressure to afford a residue, which was purified by silica gel chromatography (0-10% MeOH in DCM) to afford tert-butyl (5-(ethyl(methyl)amino)-6-(hydroxymethyl)pyridazin-3-yl)carbamate (1.70 g, crude) as a white solid.

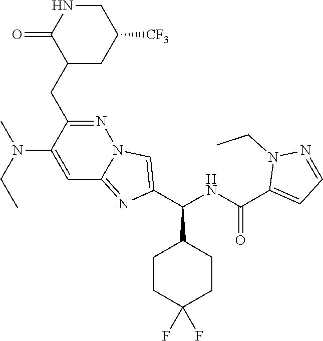 [SMILES: CCN(C)c1cc2nc([C@@H](NC(=O)c3ccnn3CC)C3CCC(F)(F)CC3)cn2nc1CC1C[C@@H](C(F)(F)F)CNC1=O]

The title compound was prepared according to General Procedure 7, employing tert-butyl (5-(ethyl(methyl)amino)-6-(hydroxymethyl)pyridazin-3-yl)carbamate, General Procedure 8, employing tert-butyl (6-(chloromethyl)-5-(ethyl(methyl)amino)pyridazin-3-yl)carb amate and 1-(tert-butyl) 3-methyl (5R)-2-oxo-5-(trifluoromethyl)piperidine-1,3-dicarboxylate, General Procedure 9, employing 1-(tert-butyl) 3-methyl (5R)-3-((6-((tert-butoxycarbonyl)amino)-4-(ethyl(methyl)amino)pyridazin-3-yl)methyl)-2-oxo-5-(trifluoromethyl)piperidine-1,3-dicarboxylate, General Procedure 1, employing methyl (5R)-3-((6-amino-4-(ethyl(methyl)amino)pyridazin-3-yl)methyl)-2-oxo-5-(trifluoromethyl)piperidine-3-carboxylate and benzyl (S)-(3-bromo-1-(4,4-difluorocyclohexyl)-2-oxopropyl)carbamate, General Procedure 5, employing methyl (5R)-3-((2-((S)-(((benzyloxy)carbonyl)amino)(4,4-difluorocyclohexyl)methyl)-7-(ethyl(methyl)amino)imidazo[1,2-b]pyridazin-6-yl)methyl)-2-oxo-5-(trifluoromethyl)piperidine-3-carboxylate, General Procedure 6, employing (5R)-3-((2-((S)-(((benzyloxy)carbonyl)amino)(4,4-difluorocyclohexyl)methyl)-7-(ethyl(methyl)amino)imidazo[1,2-b]pyridazin-6-yl)methyl)-2-oxo-5-(trifluoromethyl)piperidine-3-carboxylic acid, General Procedure 2, employing (5R)-3-((2-((S)-amino(4,4-difluorocyclohexyl)methyl)-7-(ethyl(methyl)amino)imidazo[1,2-b]pyridazin-6-yl)methyl)-5-(trifluoromethyl)piperidin-2-one, and General Procedure 4, employing N-((1S)-(4,4-difluorocyclohexyl)(7-(ethyl(methyl)amino)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide and 1-ethyl-1H-pyrazole-5-carboxylic acid, which was further purified by chiral prep-SFC to afford Compound 88 as the first eluting, single stereoisomer (35.0 mg, 55.6 μmol, LCMS [M+H]⁺=625.3 m z) as a white solid and Compound 89 as the second eluting, single stereoisomer (19.0 mg, 30.0 μmol, LCMS [M+H]⁺=625.3 m z) as a white solid.

### Example 82: Preparation of Compounds 90 and 91

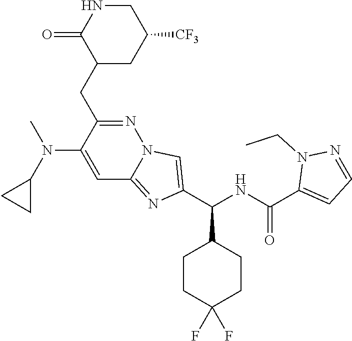 [SMILES: CCn1nccc1C(=O)N[C@H](c1cn2nc(CC3C[C@@H](C(F)(F)F)CNC3=O)c(N(C)C3CC3)cc2n1)C1CCC(F)(F)CC1]

The title compound was prepared according to General Procedure 10, employing N-methylcyclopropanamine, General Procedure 11, employing methyl 6-chloro-4-(cyclopropyl(methyl)amino)pyridazine-3-carboxylate, General Procedure 13, employing methyl 6-((tert-butoxycarbonyl)amino)-4-(cyclopropyl(methyl)amino)pyridazine-3-carboxylate, General Procedure 7, employing tert-butyl (5-(cyclopropyl(methyl)amino)-6-(hydroxymethyl)pyridazin-3-yl)carbamate, General Procedure 8, employing tert-butyl (6-(chloromethyl)-5-(cyclopropyl(methyl)amino)pyridazin-3-yl)carbamate and 1-(tert-butyl) 3-methyl (5R)-2-oxo-5-(trifluoromethyl)piperidine-1,3-dicarboxylate, General Procedure 12, employing 1-(tert-butyl) 3-methyl (5R)-3-((6-((tert-butoxycarbonyl)amino)-4-(cyclopropyl(methyl)amino)pyridazin-3-yl)methyl)-2-oxo-5-(trifluoromethyl)piperidine-1,3-dicarboxylate, General Procedure 1, employing methyl (5R)-3-((6-amino-4-(cyclopropyl(methyl)amino)pyridazin-3-yl)methyl)-2-oxo-5-(trifluoromethyl)piperidine-3-carboxylate (TFA salt) and benzyl (S)-(3-bromo-1-(4,4-difluorocyclohexyl)-2-oxopropyl)carbamate, General Procedure 5, employing methyl (5R)-3-((2-((S)-(((benzyloxy)carbonyl)amino)(4,4-difluorocyclohexyl)methyl)-7-(cyclopropyl(methyl)amino)imidazo[1,2-b]pyridazin-6-yl)methyl)-2-oxo-5-(trifluoromethyl)piperidine-3-carboxylate, General Procedure 6, employing (5R)-3-((2-((S)-(((benzyloxy)carbonyl)amino)(4,4-difluorocyclohexyl)methyl)-7-(cyclopropyl(methyl)amino)imidazo[1,2-b]pyridazin-6-yl)methyl)-2-oxo-5-(trifluoromethyl)piperidine-3-carboxylic acid, General Procedure 3, employing benzyl ((1S)-(7-(cyclopropyl(methyl)amino)-6-(((5R)-2-oxo-5-(trifluoromethyl)piperidin-3-yl)methyl)imidazo[1,2-b]pyridazin-2-yl)(4,4-difluorocyclohexyl)methyl)carbamate with ammonium formate, and General Procedure 4, employing (5R)-3-((2-((S)-amino(4,4-difluorocyclohexyl)methyl)-7-(cyclopropyl(methyl)amino)imidazo[1,2-b]pyridazin-6-yl)methyl)-5-(trifluoromethyl)piperidin-2-one and 1-ethyl-1H-pyrazole-5-carboxylic acid to afford N-((1S)-(7-(cyclopropyl(methyl)amino)-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide, which was further purified by chiral prep-SFC (REGIS (S, S) WHELK-O1 (250 mm×25 mm, 10 μm); 45% mobile phase: [MeOH (0.1% NH₃H₂O)]) to afford Compound 90 as the first eluting, single stereoisomer (30.55 mg, 47.7 μmol, LCMS [M+H]⁺=637.3 m z) as a light yellow solid and Compound 91 as the second eluting, single stereoisomer (25.86 mg, 39.8 μmol, LCMS [M+H]⁺=637.4 m z) as a light yellow solid.

### Example 83: Preparation of Compound 92

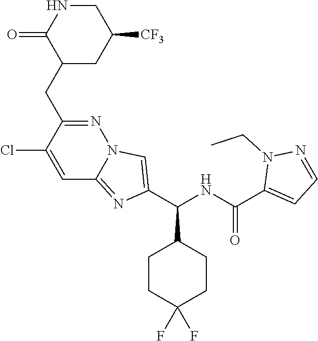 [SMILES: CCn1nccc1C(=O)N[C@H](c1cn2nc(CC3C[C@H](C(F)(F)F)CNC3=O)c(Cl)cc2n1)C1CCC(F)(F)CC1]

The title compound was prepared according to General Procedure 7, employing benzyl (S)-((7-chloro-6-(hydroxymethyl)imidazo[1,2-b]pyridazin-2-yl)(4,4-difluorocyclohexyl)methyl)carbamate, General Procedure 14, employing benzyl (S)-((7-chloro-6-(chloromethyl)imidazo[1,2-b]pyridazin-2-yl)(4,4-difluorocyclohexyl)methyl)carbamate and 1-(tert-butyl) 3-methyl (5S)-2-oxo-5-(trifluoromethyl)piperidine-1,3-dicarboxylate, General Procedure 12, employing 1-(tert-butyl) 3-methyl (5S)-3-((2-((S)-(((benzyloxy)carbonyl)amino)(4,4-difluorocyclohexyl)methyl)-7-chloroimidazo[1,2-b]pyridazin-6-yl)methyl)-2-oxo-5-(trifluoromethyl)piperidine-1,3-dicarboxylate, General Procedure 5, employing methyl (5S)-3-((2-((S)-(((benzyloxy)carbonyl)amino)(4,4-difluorocyclohexyl)methyl)-7-chloroimidazo[1,2-b]pyridazin-6-yl)methyl)-2-oxo-5-(trifluoromethyl)piperidine-3-carboxylate, General Procedure 6, employing (5S)-3-((2-((S)-(((benzyloxy)carbonyl)amino)(4,4-difluorocyclohexyl)methyl)-7-chloroimidazo[1,2-b]pyridazin-6-yl)methyl)-2-oxo-5-(trifluoromethyl)piperidine-3-carboxylic acid, General Procedure 15, employing benzyl ((1S)-(7-chloro-6-(((5S)-2-oxo-5-(trifluoromethyl)piperidin-3-yl)methyl)imidazo[1,2-b]pyridazin-2-yl)(4,4-difluorocyclohexyl)methyl)carbamate, and General Procedure 4, employing (5S)-3-((2-((S)-amino(4,4-difluorocyclohexyl)methyl)-7-chloroimidazo[1,2-b]pyridazin-6-yl)methyl)-5-(trifluoromethyl)piperidin-2-one and 1-ethyl-1H-pyrazole-5-carboxylic acid to afford N-((1S)-(7-chloro-6-(((5S)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide, (60.0 mg, 99.6 μmol) as a white solid. LCMS [M+H]⁺=602.2 m/z.

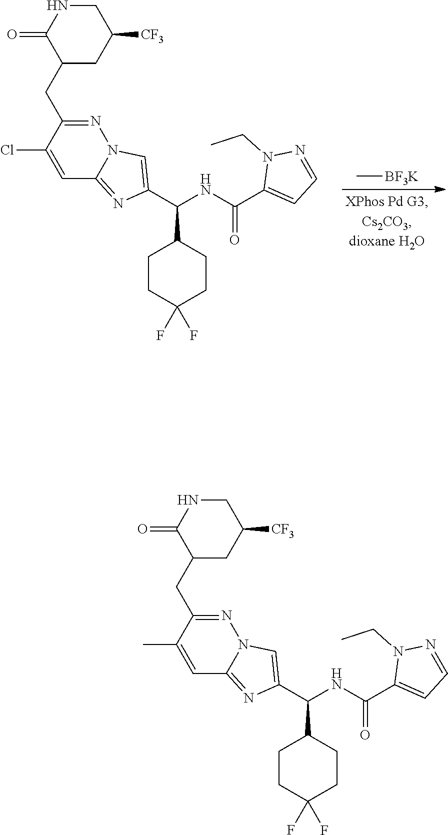

To a solution of N-((1S)-(7-chloro-6-(((5S)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (50.0 mg, 83.0 uM, 1.00 eq) and potassium methyltrifluoroborate (124 mg, 2.08 mmol, 25 eq) was added Cs₂CO₃ (81.1 mg, 249 μmol, 3.00 eq), and XPhos Pd G3 (7.03 mg, 8.31 mol, 0.100 eq) in H₂O (1.00 mL) and dioxane (8.00 mL). The reaction mixture was stirred at 100° C. for 4 h under N₂. The reaction mixture was partitioned between H₂O (10.0 mL) and DCM (30.0 mL×2). The organic phase was separated, and the combined organic layers were dried over anhydrous Na₂SO₄, filtered, and concentrated under reduced pressure to give a residue. The residue was purified by prep-TLC (10:1 DCM/MeOH) to afford N-((1S)-(4,4-difluorocyclohexyl)(7-methyl-6-(((5S)-2-oxo-5-(trifluoromethyl)piperidin-3-yl)methyl)imidazo[1,2-b]pyridazin-2-yl)methyl)-1-ethyl-1H-pyrazole-5-carboxamide (35.0 mg, 60.1 μmol, 72% yield) as a white solid, which was further purified by chiral prep-SFC (DAICEL CHIRALPAK AS (250 mm×30 mm, 10 μm); 40% mobile phase: [MeOH (0.1% NH₃H₂O)]) to afford Compound 92 as the second eluting, single stereoisomer (4.16 mg, 7.15 μmol) as a white solid. LCMS [M+H]⁺=582.4 m/z.

### Example 84: Preparation of Compounds 93 and 94

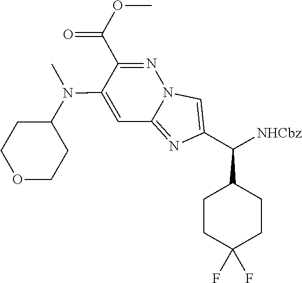 [SMILES: COC(=O)c1nn2cc([C@@H](NC(=O)OCc3ccccc3)C3CCC(F)(F)CC3)nc2cc1N(C)C1CCOCC1]

Methyl (S)-2-((((benzyloxy)carbonyl)amino)(4,4-difluorocyclohexyl)methyl)-7-(methyl(tetrahydro-2H-pyran-4-yl)amino)imidazo[1,2-b]pyridazine-6-carboxylate was synthesized according to General Procedure 10, employing N-methyltetrahydro-2H-pyran-4-amine, General Procedure 11, employing methyl 6-chloro-4-(methyl(tetrahydro-2H-pyran-4-yl)amino)pyridazine-3-carboxylate, General Procedure 12, employing methyl 6-((tert-butoxycarbonyl)amino)-4-(methyl(tetrahydro-2H-pyran-4-yl)amino)pyridazine-3-carboxylate, and General Procedure 1, employing methyl 6-amino-4-(methyl(tetrahydro-2H-pyran-4-yl)amino)pyridazine-3-carboxylate and benzyl (S)-(3-bromo-1-(4,4-difluorocyclohexyl)-2-oxopropyl)carbamate.

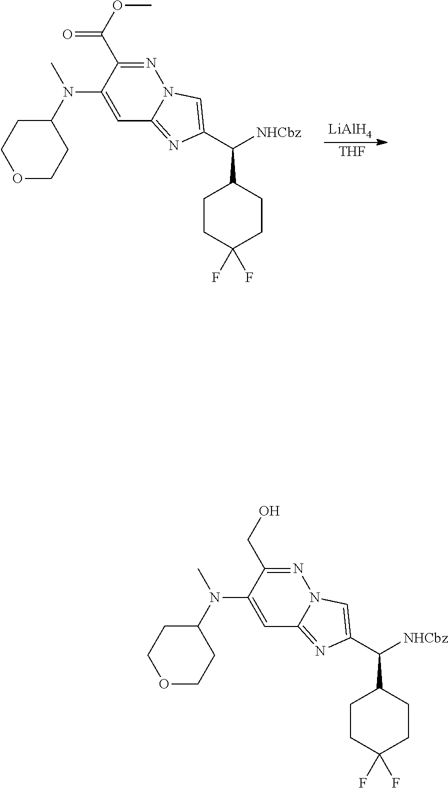 [SMILES: COC(=O)c1nn2cc([C@@H](NC(=O)OCc3ccccc3)C3CCC(F)(F)CC3)nc2cc1N(C)C1CCOCC1 ; "LiAlH4" ; "THF" ; CN(c1cc2nc([C@@H](NC(=O)OCc3ccccc3)C3CCC(F)(F)CC3)cn2nc1CO)C1CCOCC1]

To a solution of methyl (S)-2-((((benzyloxy)carbonyl)amino)(4,4-difluorocyclohexyl)methyl)-7-(methyl(tetrahydro-2H-pyran-4-yl)amino)imidazo[1,2-b]pyridazine-6-carboxylate (1.50 g, 2.62 mmol, 1.00 eq) in THF (10.0 mL) was added LiAlH₄ (2.50M, 1.57 mL, 1.50 eq). The reaction mixture was stirred at 0° C. for 30 min. After the reaction mixture was cooled to 0° C., the reaction mixture was treated with 0.150 mL of H₂O, followed by 0.150 mL of 15% aqueous NaOH and 0.450 mL of H₂O. After stirring at RT for 15 min, the reaction mixture was dried over anhydrous MgSO₄. The reaction mixture was filtered, concentrated, and purified by silica gel chromatography (PE followed by 10:1 DCM/MeOH to afford benzyl (S)-((4,4-difluorocyclohexyl)(6-(hydroxymethyl)-7-(methyl(tetrahydro-2H-pyran-4-yl)amino)imidazo[1,2-b]pyridazin-2-yl)methyl)carbamate (500 mg, 919 μmol, 35% yield) as a yellow solid. LCMS [M+H]⁺=544.4 m/z.

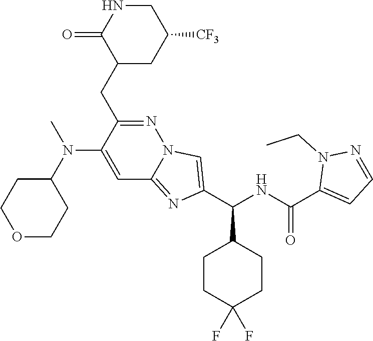 [SMILES: CCn1nccc1C(=O)N[C@H](c1cn2nc(CC3C[C@@H](C(F)(F)F)CNC3=O)c(N(C)C3CCOCC3)cc2n1)C1CCC(F)(F)CC1]

The title compound was prepared according to General Procedure 7, employing benzyl (S)-((4,4-difluorocyclohexyl)(6-(hydroxymethyl)-7-(methyl(tetrahydro-2H-pyran-4-yl)amino)imidazo[1,2-b]pyridazin-2-yl)methyl)carbamate, General Procedure 14, employing benzyl (S)-((6-(chloromethyl)-7-(methyl(tetrahydro-2H-pyran-4-yl)amino)imidazo[1,2-b]pyridazin-2-yl)(4,4-difluorocyclohexyl)methyl)carbamate and methyl (5R)-2-oxo-5-(trifluoromethyl)piperidine-3-carboxylate, General Procedure 5, employing (5R)-3-((2-((S)-(((benzyloxy)carbonyl)amino)(4,4-difluorocyclohexyl)methyl)-7-(methyl(tetrahydro-2H-pyran-4-yl)amino)imidazo[1,2-b]pyridazin-6-yl)methyl)-2-oxo-5-(trifluoromethyl)piperidine-3-carboxylic acid, General Procedure 6, employing methyl (5R)-3-((2-((S)-(((benzyloxy)carbonyl)amino)(4,4-difluorocyclohexyl)methyl)-7-(methyl(tetrahydro-2H-pyran-4-yl)amino)imidazo[1,2-b]pyridazin-6-yl)methyl)-2-oxo-5-(trifluoromethyl)piperidine-3-carboxylate, General Procedure 2, employing benzyl ((1S)-(4,4-difluorocyclohexyl)(7-(methyl(tetrahydro-2H-pyran-4-yl)amino)-6-(((5R)-2-oxo-5-(trifluoromethyl)piperidin-3-yl)methyl)imidazo[1,2-b]pyridazin-2-yl)methyl)carbamate, and General Procedure 4, employing (5R)-3-((2-((S)-amino(4,4-difluorocyclohexyl)methyl)-7-(methyl(tetrahydro-2H-pyran-4-yl)amino)imidazo[1,2-b]pyridazin-6-yl)methyl)-5-(trifluoromethyl)piperidin-2-one and 1-ethyl-1H-pyrazole-5-carboxylic acid to afford N-((1S)-(4,4-difluorocyclohexyl)(7-(methyl(tetrahydro-2H-pyran-4-yl)amino)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, which was further purified by chiral prep-SFC (REGIS(S, S) WHELK-O₁ (250 mm×25 mm, 10 μm); 55% mobile phase: [EtOH (0.1% NH₃H₂O)]) to afford Compound 93 as the first eluting, single stereoisomer (26.06 mg, 35.9 μmol, LCMS [M+H]⁺=681.4 m z) as a light yellow solid and Compound 94 as the second eluting, single stereoisomer (21.0 mg, 30.0 μmol, LCMS [M+H]⁺=681.4 m z) as a light yellow solid.

### Example 85: Preparation of Intermediate 4

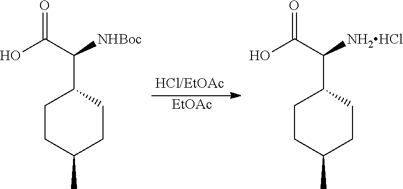

To a solution of (S)-2-((tert-butoxycarbonyl)amino)-2-((1R,4S)-4-methylcyclohexyl)acetic acid (250 g, 921 mmol, 1.00 eq) in EtOAc (500 mL) was added a solution of HCl in EtOAc (4 M, 2.5 L, 10.8 eq). The reaction mixture was stirred at a temperature maintained between 0 and 10° C. for 2 h. The reaction mixture was then concentrated under reduced pressure to afford (S)-2-amino-2-((1R,4S)-4-methylcyclohexyl)acetic acid (200 g, crude, HCl salt) as a white solid.

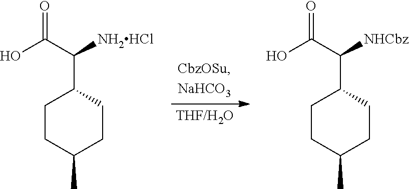

To a solution of (S)-2-amino-2-((1R,4S)-4-methylcyclohexyl)acetic acid (193 g, 934 mmol, 1.00 eq) in THE (800 mL) and water (800 mL) was added potassium carbonate (387 g, 2.80 μmol, 3.00 eq) followed by N-(Benzyloxycarbonyloxy)succinimide (233 g, 934 mmol, 1.00 eq). The reaction mixture was stirred at a temperature maintained between 10 and RT for 12 h. The reaction mixture was extracted twice with EtOAc. The combined organic layers were washed with brine, dried over anhydrous Na₂SO₄, filtered and concentrated under reduced pressure to give a residue, which was triturated with isopropanol at RT for 30 min to afford (S)-2-(((benzyloxy)carbonyl)amino)-2-((1R,4S)-4-methylcyclohexyl)acetic acid (200 g, 633 mmol, 68% yield) as a white solid. LCMS [M+Na]⁺=328.2 m/z.

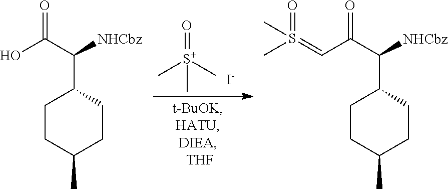

To a solution of trimethylsulfoxoniumiodide (648 g, 2.95 μmol, 3.00 eq) in THF (1500 mL) was added potassium tert-butoxide (330 g, 2.95 μmol, 3.00 eq). The reaction mixture was stirred at 80° C. for 2 h (Reaction 1).

In parallel, to a solution of (S)-2-(((benzyloxy)carbonyl)amino)-2-((1R,4S)-4-methylcyclohexyl)acetic acid (300 g, 982 mmol, 1.00 eq) in THF (1500 mL) was added DIEA (165 g, 1.28 μmol, 222 mL, 1.30 eq) followed by 1-[bis(dimethylamino)methylene]-1H-1,2,3-triazolo[4,5-b]pyridinium 3-oxide hexafluorophosphate (HATU, 448 g, 1.18 μmol, 1.20 eq). The reaction mixture was stirred at RT for 2 h (Reaction 2).

The content of the Reaction 2 was slowly added to the Reaction 1 at RT and the resulting mixture was further stirred at RT for 2 h. The reaction mixture was quenched by the addition of water and then diluted with the addition of EtOAc. An extraction was performed twice with EtOAc. The combined organic layers were washed with brine, dried over anhydrous Na₂SO₄, filtered and concentrated under reduced pressure to give a residue, which was purified by column chromatography (SiO₂, PE/EtOAc) to afford benzyl ((S)-3-(dimethyl(oxo)-l⁶-sulfaneylidene)-1-((1R,4S)-4-methylcyclohexyl)-2-oxopropyl)carbamate (400 g, crude) as an off-white solid. LCMS [M+H]⁺=380.1 m/z.

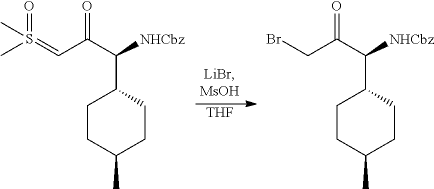 [SMILES: CS(C)(=O)=CC(=O)[C@@H](NC(=O)OCc1ccccc1)[C@H]1CC[C@H](C)CC1 ; C[C@H]1CC[C@H]([C@H](NC(=O)OCc2ccccc2)C(=O)CBr)CC1 ; "THF"]

To a solution of benzyl ((S)-3-(dimethyl(oxo)-l⁶-sulfaneylidene)-1-((1R,4S)-4-methylcyclohexyl)-2-oxopropyl)carbamate (150 g, 395 mmol, 1.00 eq) in THF (1500 mL) was added LiBr (34.3 g, 395 mmol, 1.00 eq) followed by methanesulfonic acid (38.0 g, 395 mmol, 28.1 mL, 1.00 eq). The reaction mixture was stirred at 45° C. for 12 h. The reaction mixture was quenched by the addition of water, and then extracted twice with EtOAc. The combined organic layers were washed with brine, dried over anhydrous Na₂SO₄, filtered and concentrated under reduced pressure to give a residue, which was purified by prep-HPLC (Phenomenexluna C18 250×100 mm, 10 μm; mobile phase: [water (FA)-ACN]; B %: 60%-80%) to afford Intermediate 4, benzyl ((S)-3-bromo-1-((1R,4S)-4-methylcyclohexyl)-2-oxopropyl)carbamate, (18.2 g, 47.8 mmol, 12% yield) as a white solid. ¹H NMR (400 MHz, CDCl₃) δ 7.31-7.37 (m, 5H), 5.31-5.33 (m, 1H), 5.09 (s, 2H), 4.58-4.60 (m, 1H), 3.99-4.10 (m, 2H), 1.65-1.80 (m, 4H), 1.42-1.48 (m, 1H), 1.25-1.40 (m, 2H), 0.91-1.10 (m, 3H), 0.86-0.88 (m, 3H). LCMS [M+H]⁺=382.1 m/z.

### Example 86: Preparation of Intermediates 5 and 6

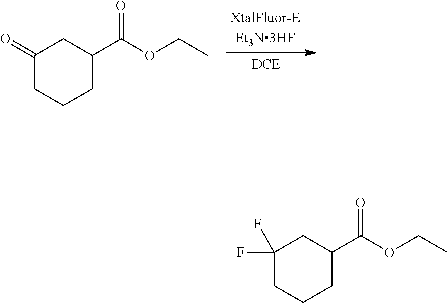

To a solution of N,N-diethylamino-S,S-difluorosulfinium tetrafluoroborate (5.05 kg, 22.0 mol, 2.50 eq) and triethylamine trihydrofluoride (2.13 kg, 13.2 μmol, 2.15 L, 1.50 eq) in 1,2-dichloroethane (25.0 L) was added dropwise ethyl 3-oxocyclohexane-1-carboxylate (1.50 kg, 8.81 mol, 1.00 eq) over a period of 30 min. The reaction mixture was stirred at 80° C. for 3 h. The reaction mixture was poured into a saturated aqueous solution of NaCO₃ (15.0 L) at RT and the organic phase was separated. The aqueous phase was extracted with DCM, the combined organic phases were washed with a 10% aqueous solution of citric acid and brine. Upon concentration under reduced pressure to give a residue, a distillation under vacuum (80° C., −0.095 Mpa) was performed to afford ethyl 3,3-difluorocyclohexane-1-carboxylate (1.26 kg, 6.56 μmol, 74% yield) as a yellow oil. ¹H NMR (400 MHz, CDCl₃) δ 4.15 (q, J=7.2 Hz, 2H), 2.57-2.64 (m, 1H), 2.30-2.37 (m, 1H), 1.99-2.12 (m, 2H), 1.78-1.94 (m, 2H), 1.53-1.73 (m, 2H), 1.35-1.45 (m, 1H), 1.26 (t, J=7.2 Hz, 3H).

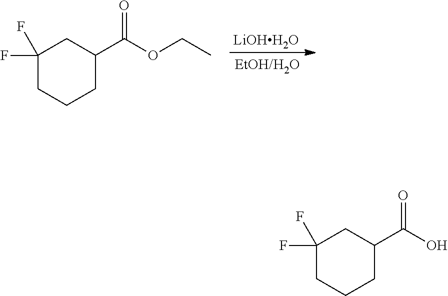

To a solution of ethyl 3,3-difluorocyclohexane-1-carboxylate (1.26 kg, 6.56 μmol, 1.00 eq) in ethanol (7.56 L) and water (5.04 L) was added portionwise LiOH·H₂O (330 g, 7.88 μmol, 1.20 eq) at 0° C. The reaction mixture was stirred at RT for 2 h. The reaction mixture was concentrated under reduced pressure. The resulting residue was diluted with a 3M aqueous solution of HCl to adjust pH=3 and extracted with DCM. The combined organic phases were dried over anhydrous Na₂SO₄, filtered, and concentrated under reduced pressure to give a residue, which was stirred in PE at RT for 5 h to afford 3,3-difluorocyclohexane-1-carboxylic acid (655 g, 3.99 μmol, 61% yield) as a white solid. ¹H NMR (400 MHz, CDCl₃) δ 2.66-2.72 (m, 1H), 2.33-2.41 (m, 1H), 2.06-2.14 (m, 2H), 1.81-1.97 (m, 2H), 1.57-1.76 (s, 2H), 1.40-1.49 (m, 1H).

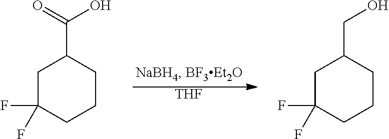 [SMILES: O=C(O)C1CCCC(F)(F)C1 ; OCC1CCCC(F)(F)C1 ; "THF"]

To a solution of 3,3-difluorocyclohexane-1-carboxylic acid (50.0 g, 305 mmol, 1.00 eq) in THE (600 mL) was added portionwise NaBH₄ (12.7 g, 335 mmol, 1.10 eq) at 0° C. followed by a dropwise addition of boron trifluoride diethyl etherate (43.2 g, 305 mmol, 37.5 mL, 1.00 eq). The reaction mixture was stirred at RT for 3 h. The reaction mixture was quenched by the addition of a saturated aqueous solution of ammonium chloride and extracted with EtOAc, dried over anhydrous Na₂SO₄, filtered and concentrated under reduced pressure to afford (3,3-difluorocyclohexyl) MeOH (45.0 g, 300 mmol, 98% yield) as a yellow oil. ¹H NMR (400 MHz, DMSO-d₆) δ 4.56 (t, J=6.4 Hz, 1H), 3.22-3.32 (m, 2H), 2.00-2.07 (m, 1H), 1.93-1.98 (m, 1H), 1.58-1.79 (m, 4H), 1.35-1.51 (m, 2H), 0.95-1.02 (m, 1H).

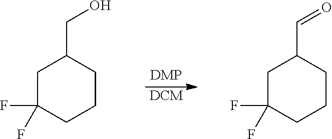

To a solution of (3,3-difluorocyclohexyl) MeOH (40.0 g, 266 mmol, 1.00 eq) in DCM (1.00 L) was added Dess-Martin periodinane (124 g, 293 mmol, 90.8 mL, 1.10 eq) at 0° C. The reaction mixture was stirred at RT for 1 h. The reaction mixture was concentrated under reduced pressure and the resulting residue was purified by re-crystallization from PE at RT to afford 3,3-difluorocyclohexane-1-carbaldehyde (39.0 g, 263 mmol, 99% yield) as a yellow oil. ¹H NMR (400 MHz, CDCl₃) δ 9.67 (d, J=1.6 Hz, 1H), 2.62-2.71 (m, 1H), 2.31-2.40 (m, 1H), 1.97-2.06 (m, 2H), 1.75-1.92 (m, 4H), 1.51-1.57 (m, 1H).

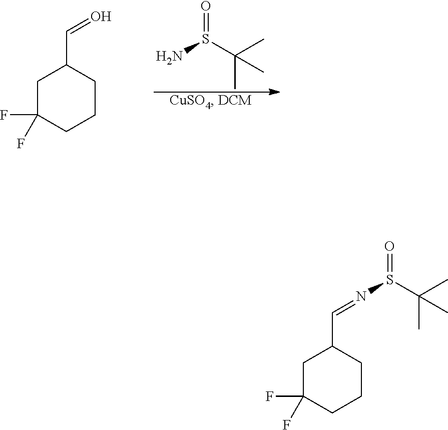

To a solution of 3,3-difluorocyclohexane-1-carbaldehyde (39.0 g, 263 mmol, 1.00 eq) and (S)-2-methylpropane-2-sulfinamide (38.2 g, 316 mmol, 1.20 eq) in DCM (600 mL) was added copper sulfate (42.0 g, 263 mmol, 40.4 mL, 1.00 eq). The reaction mixture was stirred at RT for 10 h. The reaction mixture was diluted with water and extracted with DCM. The combined organic layers were washed with brine, dried over anhydrous Na₂SO₄, filtered and concentrated under reduced pressure to afford (S)—N-((3,3-difluorocyclohexyl)methylene)-2-methylpropane-2-sulfinamide (49.0 g, 195 mmol, 74% yield) as a yellow oil. ¹H NMR (400 MHz, CDCl₃) δ 8.00-8.02 (m, 1H), 2.75-2.82 (m, 1H), 2.30-2.36 (m, 1H), 2.11-2.13 (m, 1H), 1.87-1.93 (m, 1H), 1.53-1.84 (m, 5H), 1.18 (s, 9H).

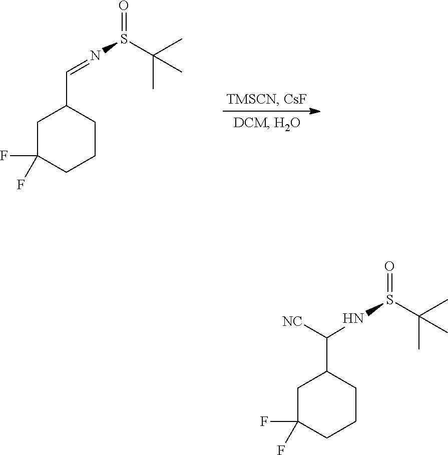 [SMILES: CC(C)(C)[S@](=O)N=CC1CCCC(F)(F)C1 ; CC(C)(C)[S@](=O)NC(C#N)C1CCCC(F)(F)C1]

To a solution of (S)—N-((3,3-difluorocyclohexyl)methylene)-2-methylpropane-2-sulfinamide (49.0 g, 195 mmol, 1.00 eq) in DCM (600 mL) was successively added water (7.00 g, 389 mmol, 7.00 mL, 1.99 eq), cesium fluoride (5.92 g, 39.0 mmol, 1.44 mL, 0.200 eq) and trimethylsilyl cyanide (39.0 g, 390 mmol, 49.0 mL, 2.00 eq). The reaction mixture was stirred at RT for 5 h. The reaction mixture was quenched by the addition of water at 0° C., and an extraction with DCM was performed. The combined organic layers were washed with brine, dried over anhydrous Na₂SO₄, filtered and concentrated under reduced pressure to give a residue which was purified by column chromatography (SiO₂, PE/EtOAc) to afford (S)—N-(cyano(3,3-difluorocyclohexyl)methyl)-2-methylpropane-2-sulfinamide (49.0 g, 176 mmol, 90% yield) as a yellow oil. ¹H NMR (400 MHz, CDCl₃) δ 4.11-4.16 (m, 1H), 2.27-2.30 (m, 1H), 2.11-2.20 (m, 2H), 1.89-2.02 (m, 2H), 1.38-1.75 (m, 4H), 1.26 (s, 9H).

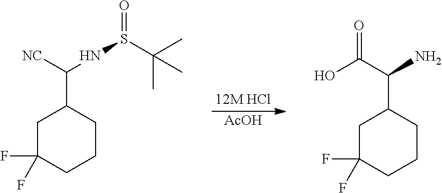 [SMILES: "AcOH" ; CC(C)(C)[S@](=O)NC(C#N)C1CCCC(F)(F)C1 ; N[C@H](C(=O)O)C1CCCC(F)(F)C1]

To a solution of (S)—N-(cyano(3,3-difluorocyclohexyl)methyl)-2-methylpropane-2-sulfinamide (49.0 g, 176 mmol, 1.00 eq) in acetic acid (250 mL) was added HCl (12 M, 250 mL, 17.0 eq). The reaction mixture was stirred at 110° C. for 12 h. The reaction mixture was concentrated under reduced pressure to afford (2S)-2-amino-2-(3,3-difluorocyclohexyl)acetic acid (31.0 g, 160 mmol, 91% yield) as a brown solid.

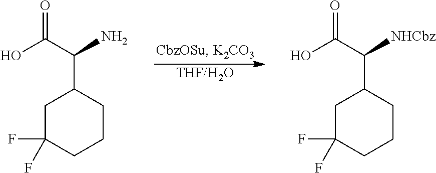 [SMILES: N[C@H](C(=O)O)C1CCCC(F)(F)C1 ; "THF/H2O" ; O=C(N[C@H](C(=O)O)C1CCCC(F)(F)C1)OCc1ccccc1]

To a solution of (2S)-2-amino-2-(3,3-difluorocyclohexyl)acetic acid (31.0 g, 160 mmol, 1.00 eq) in THE (250 mL) and water (250 mL) was added potassium carbonate (66.5 g, 481 mmol, 3.00 eq) followed by N-(Benzyloxycarbonyloxy)succinimide (44.0 g, 176 mmol, 1.10 eq). The reaction mixture was stirred at RT for 10 h. The reaction mixture was concentrated under reduced pressure and the resulting residue was diluted with a 1 M aqueous solution of HCl to adjust pH=3, then extracted with DCM. The combined organic layers were washed with brine, dried over anhydrous Na₂SO₄, filtered, and concentrated under reduced pressure to give a residue which was purified by prep-HPLC (0.1% FA condition) to afford (2S)-2-(((benzyloxy)carbonyl)amino)-2-(3,3-difluorocyclohexyl)acetic acid (40.0 g, 122 mmol, 76% yield) as a white solid. LCMS [M+H]⁺=328.2 m/z.

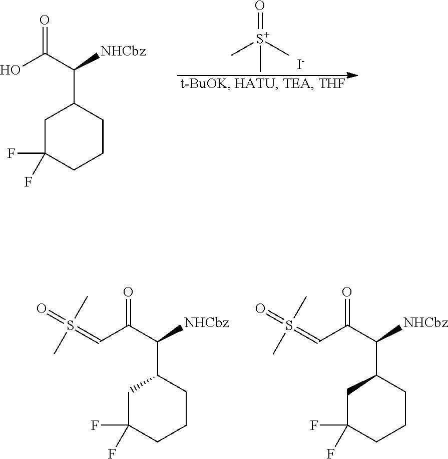

To a solution of trimethylsulfoxonium iodide (66.6 g, 302 mmol, 3.00 eq) in THF (400 mL) was added a solution of potassium tert-butoxide (33.9 g, 302 mmol, 3.00 eq) in THE (400 mL). The reaction mixture was stirred at 65° C. for 2 h and then cooled down to 0° C. (Reaction 1).

In parallel, to a solution of (2S)-2-(((benzyloxy)carbonyl)amino)-2-(3,3-difluorocyclohexyl)acetic acid (33.0 g, 101 mmol, 1.00 eq) in THF (300 mL) was added triethylamine (13.3 g, 131 mmol, 18.2 mL, 1.30 eq) followed by 1-[bis(dimethylamino)methylene]-1H-1,2,3-triazolo[4,5-b]pyridinium 3-oxide hexafluorophosphate (HATU, 49.8 g, 131 mmol, 1.30 eq) at 0° C. The reaction mixture was stirred at RT for 2 h. The suspension was then filtered, and the filtrate was cooled to 0° C. (Reaction 2). The contents of Reaction 2 were slowly added to Reaction 1 at 0° C., and the resulting mixture was further stirred at 0° C. for 2 h. The reaction mixture was quenched by the addition of water and then diluted with EtOAc. An extraction was performed with EtOAc. The combined organic layers were dried over anhydrous Na₂SO₄, filtered, and concentrated under reduced pressure to give a residue, which was purified by prep-HPLC (0.1% FA condition) to afford benzyl ((IS)-1-(3,3-difluorocyclohexyl)-3-(dimethyl(oxo)-l⁶-sulfaneylidene)-2-oxopropyl)carbamate (20.0 g, 49.2 mmol, 49% yield) as a white solid. LCMS [M+H]⁺=402.3 m/z.

Benzyl ((S)-1-((R)-3,3-difluorocyclohexyl)-3-(dimethyl(oxo)-l⁶-sulfaneylidene)-2-oxopropyl)carbamate was isolated as the first eluting, single stereoisomer by chiral SFC purification (Daicel ChiralpakIF (250 mm×30 mm, 10 mm); mobile phase: [CO₂—0.1% NH₃H₂O in EtOH]; B %: 3 5%); (9.00 g, 22.4 mmol, 47% yield) and was obtained as a white solid. LCMS [M+H]⁺=402.2 m/z.

Benzyl ((S)-1-((S)-3,3-difluorocyclohexyl)-3-(dimethyl(oxo)-l⁶-sulfaneylidene)-2-oxopropyl)carbamate was isolated as the second eluting, single stereoisomer by chiral SFC purification (Daicel ChiralpakIF (250 mm×30 mm, 10 mm); mobile phase: [CO₂—0.1% NH₃H₂O in EtOH]; B %: 3 5%); (9.00 g, 22.4 mmol, 47% yield) and was obtained as a white solid. LCMS [M+H]⁺=402.2 m/z.

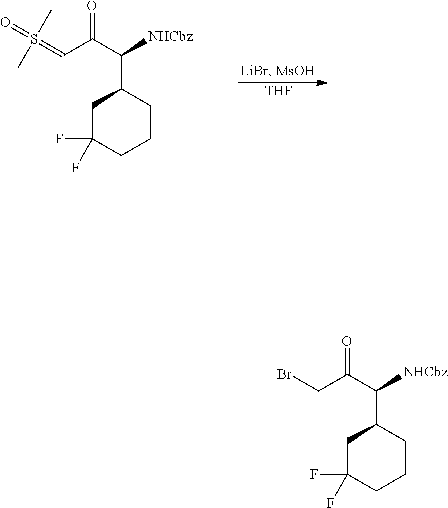 [SMILES: CS(C)(=O)=CC(=O)[C@@H](NC(=O)OCc1ccccc1)[C@H]1CCCC(F)(F)C1 ; O=C(N[C@H](C(=O)CBr)[C@H]1CCCC(F)(F)C1)OCc1ccccc1 ; "THF"]

To a solution of benzyl ((S)-1-((S)-3,3-difluorocyclohexyl)-3-(dimethyl(oxo)-l⁶-sulfaneylidene)-2-oxopropyl)carbamate (4.00 g, 9.96 mmol, 1.00 eq) in THE (15.0 mL) was added LiBr (1.30 g, 14.9 mmol, 375 μL, 1.50 eq) followed by methanesulfonic acid (1.44 g, 14.9 mmol, 1.07 mL, 1.50 eq). The reaction mixture was stirred at 45° C. for 1 h. The reaction mixture was filtered and concentrated under reduced pressure to give a residue, which was purified by column chromatography (SiO₂, PE/EtOAc) to afford Intermediate 5, benzyl ((S)-3-bromo-1-((S)-3,3-difluorocyclohexyl)-2-oxopropyl)carbamate, (3.30 g, 8.16 mmol, 82% yield) as a white solid. ¹H NMR (400 MHz, CDCl₃) δ 7.27-7.41 (m, 5H), 5.34-5.42 (m, 1H), 5.12 (s, 2H), 4.71-4.79 (m, 1H), 3.99-4.08 (m, 1H), 3.77 (s, 1H), 2.12-2.15 (m, 2H), 1.75-1.84 (m, 2H), 1.59-1.67 (m, 1H), 1.38-1.55 (m, 2H), 0.82-1.29 (m, 2H).

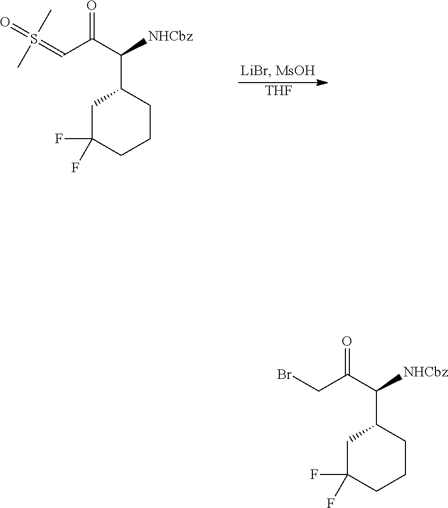 [SMILES: CS(C)(=O)=CC(=O)[C@@H](NC(=O)OCc1ccccc1)[C@@H]1CCCC(F)(F)C1 ; O=C(N[C@H](C(=O)CBr)[C@@H]1CCCC(F)(F)C1)OCc1ccccc1 ; "THF"]

To a solution of benzyl ((S)-1-((R)-3,3-difluorocyclohexyl)-3-(dimethyl(oxo)-l⁶-sulfaneylidene)-2-oxopropyl)carbamate (5.00 g, 12.45 mmol, 1.00 eq) in THF (30.0 mL) was added LiBr (1.60 g, 18.7 mmol, 471 μL, 1.50 eq) followed by methanesulfonic acid (1.81 g, 18.7 mmol, 1.34 mL, 1.50 eq). The reaction mixture was stirred at 45° C. for 1 h. The reaction mixture was filtered and concentrated under reduced pressure to give a residue, which was purified by column chromatography (SiO₂, PE/EtOAc) to afford Intermediate 6, benzyl ((S)-3-bromo-1-((R)-3,3-difluorocyclohexyl)-2-oxopropyl)carbamate, (3.40 g, 8.41 mmol, 67% yield) as a white solid. ¹H NMR (400 MHz, CDCl₃) δ 7.33-7.41 (m, 5H), 5.35-5.39 (m, 1H), 5.13 (s, 2H), 4.71-4.79 (m, 1H), 3.99-4.07 (m, 2H), 2.21-2.26 (m, 2H), 1.81-1.86 (m, 1H), 1.45-1.67 (m, 5H), 0.98-1.08 (m, 1H).

### Example 87: Preparation of Intermediates 7 and 8

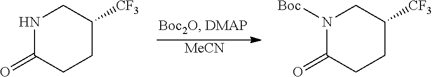 [SMILES: "MeCN" ; O=C1CC[C@@H](C(F)(F)F)CN1 ; CC(C)(C)OC(=O)N1C[C@H](C(F)(F)F)CCC1=O]

To a solution of (R)-5-(trifluoromethyl)piperidin-2-one (6.60 g, 39.5 mmol, 1.00 eq) in acetonitrile (50.0 mL) was added successively di-tert-butyl dicarbonate (10.3 g, 47.4 mmol, 10.9 mL, 1.20 eq) and 4-dimethylaminopyridine (965 mg, 7.90 mmol, 0.20 eq). The reaction mixture was stirred at RT for 3 h. The reaction mixture was concentrated under reduced pressure to give a residue, which was purified by column chromatography (SiO₂, PE:EtOAc) to afford tert-butyl (R)-2-oxo-5-(trifluoromethyl)piperidine-1-carboxylate (8.00 g, 29.9 mmol, 76% yield) as a white solid. LCMS [M+Na]⁺=290.1 m/z.

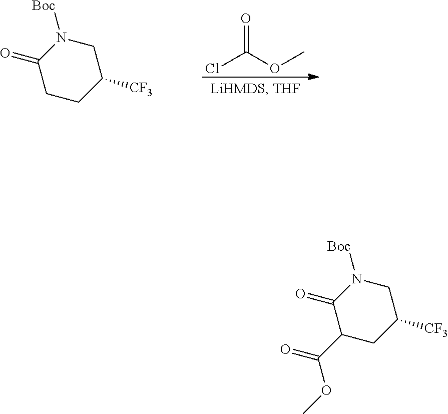

To a solution of tert-butyl (R)-2-oxo-5-(trifluoromethyl)piperidine-1-carboxylate (8.00 g 29.9 mmol, 1.00 eq) in THF (100 mL) was added a solution of lithium bis(trimethylsilyl)amide (1 M, 59.9 mL, 2.00 eq) at −78° C. The reaction mixture was stirred at −78° C. for 1 h and methyl chloroformate (5.45 g, 57.7 mmol, 4.47 mL, 1.93 eq) was then added. The reaction mixture was stirred at −78° C. for 2 h. The reaction mixture was quenched by the addition of a saturated aqueous solution of ammonium chloride at 0° C. and then extracted with EtOAc. The combined organic layers were dried over anhydrous Na₂SO₄, filtered and concentrated under reduced pressure to give a residue, which was purified by column chromatography (SiO₂, PE:EtOAc) to afford Intermediate 7, 1-(tert-butyl) 3-methyl (5R)-2-oxo-5-(trifluoromethyl)piperidine-1,3-dicarboxylate, (7.00 g, 21.5 mmol, 72% yield) as a white solid. LCMS [2M+Na]⁺=673.2 m/z.

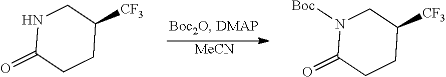 [SMILES: "MeCN" ; O=C1CC[C@H](C(F)(F)F)CN1 ; CC(C)(C)OC(=O)N1C[C@@H](C(F)(F)F)CCC1=O]

To a solution of (S)-5-(trifluoromethyl)piperidin-2-one (1.50 g, 8.98 mmol, 1.00 eq) in acetonitrile (10.0 mL) was added successively di-tert-butyl dicarbonate (2.94 g, 13.4 mmol, 3.09 mL, 1.50 eq) and 4-dimethylaminopyridine (219 mg, 1.80 mmol, 0.20 eq). The reaction mixture was stirred at RT for 12 h. The reaction mixture was concentrated under reduced pressure to give a residue, which was purified by column chromatography (SiO₂, PE:EtOAc) to afford tert-butyl (S)-2-oxo-5-(trifluoromethyl)piperidine-1-carboxylate (1.30 g, 4.86 mmol, 54% yield) as a white solid. ¹H NMR (400 MHz, CDCl₃) δ 3.99 (dd, J1=5.8, J2=13.6 Hz, 1H), 3.75 (dd, J=8.4, 13.6 Hz, 1H), 2.77-2.42 (m, 3H), 2.18-2.11 (m, 1H), 2.00-1.86 (m, 1H), 1.53 (s, 9H).

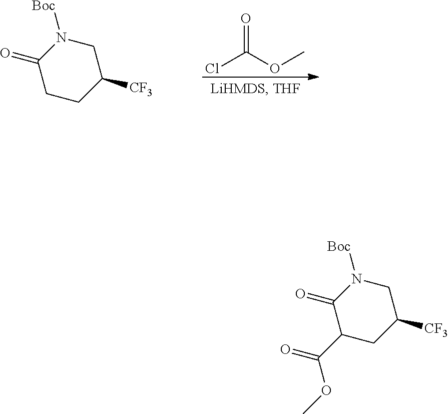

To a solution of tert-butyl (S)-2-oxo-5-(trifluoromethyl)piperidine-1-carboxylate (1.30 g 4.86 mmol, 1.00 eq) in THE (10 mL) was added a solution of lithium bis(trimethylsilyl)amide (1 M, 7.30 mL, 1.50 eq) at −70° C. The reaction mixture was stirred at −70° C. for 30 min and methyl chloroformate (689 mg, 7.30 mmol, 565 μL, 1.50 eq) was then added. The reaction mixture was stirred at −70° C. for 1 h. The reaction mixture was quenched by the addition of a saturated aqueous solution of ammonium chloride at 0° C. and then extracted with EtOAc. The combined organic layers were dried over anhydrous Na₂SO₄, filtered and concentrated under reduced pressure to give a residue, which was purified by column chromatography (SiO₂, PE:EtOAc) to afford Intermediate 8, 1-(tert-butyl) 3-methyl (5S)-2-oxo-5-(trifluoromethyl)piperidine-1,3-dicarboxylate (1.30 g, 4.00 mmol, 82% yield) as a white solid. ¹H NMR (400 MHz, CDCl₃) δ 4.03-3.93 (m, 1H), 3.88-3.97 (m, 3H), 3.77-3.49 (m, 1H), 2.95-2.58 (m, 1H), 2.57-2.08 (m, 2H), 1.53 (d, J=2.4 Hz, 9H). LCMS [2M+Na]⁺=673.2 m/z.

### Example 88: Preparation of Intermediate 9

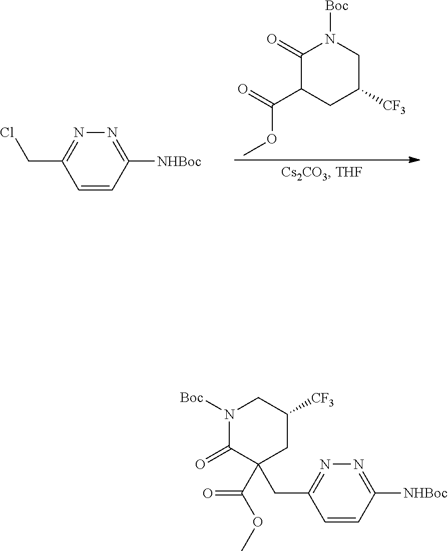 [SMILES: CC(C)(C)OC(=O)Nc1ccc(CCl)nn1 ; COC(=O)C1C[C@@H](C(F)(F)F)CN(C(=O)OC(C)(C)C)C1=O ; COC(=O)C1(Cc2ccc(NC(=O)OC(C)(C)C)nn2)C[C@@H](C(F)(F)F)CN(C(=O)OC(C)(C)C)C1=O]

To a solution of 1-(tert-butyl) 3-methyl (5R)-2-oxo-5-(trifluoromethyl)piperidine-1,3-dicarboxylate (23328, 4.50 g, 18.4 mmol, 1.20 eq) in THF (100 mL) was added Cs₂CO₃ (10.0 g 30.7 mmol, 2.00 eq). The reaction mixture was stirred at RT for 0.50 h, then tert-butyl (6-(chloromethyl)pyridazin-3-yl)carbamate (5.00 g, 15.4 mmol, 1.00 eq) was added. The reaction mixture was stirred at 70° C. for 1 h. The reaction mixture was diluted with water and extracted with EtOAc, dried over anhydrous Na₂SO₄, filtered and concentrated under reduced pressure to give a residue, which was purified by column chromatography (SiO2, PE/EtOAc) to afford 1-(tert-butyl) 3-methyl (5R)-3-((6-((tert-butoxycarbonyl)amino)pyridazin-3-yl)methyl)-2-oxo-5-(trifluoromethyl)piperidine-1,3-dicarboxylate (5.00 g, 9.39 mmol, 61% yield) as a yellow oil. LCMS [M+H]⁺=533.1 m/z.

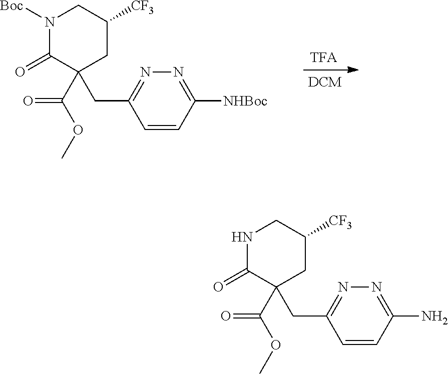 [SMILES: COC(=O)C1(Cc2ccc(NC(=O)OC(C)(C)C)nn2)C[C@@H](C(F)(F)F)CN(C(=O)OC(C)(C)C)C1=O ; COC(=O)C1(Cc2ccc(N)nn2)C[C@@H](C(F)(F)F)CNC1=O]

To a solution of 1-(tert-butyl) 3-methyl (5R)-3-((6-((tert-butoxycarbonyl)amino)pyridazin-3-yl)methyl)-2-oxo-5-(trifluoromethyl)piperidine-1,3-dicarboxylate (5.00 g, 9.39 mmol, 1.00 eq) in DCM (30.0 mL) was added TFA (46.2 g, 405 mmol, 30.0 mL, 43 eq) at 0° C. The reaction mixture was stirred at RT for 3 h. The reaction mixture was concentrated under reduced pressure to afford methyl (5R)-3-((6-aminopyridazin-3-yl)methyl)-2-oxo-5-(trifluoromethyl)piperidine-3-carboxylate (4.00 g, 8.96 mmol, 95% yield, TFA salt) as a yellow oil. LCMS [M+H]⁺=333.1 m/z.

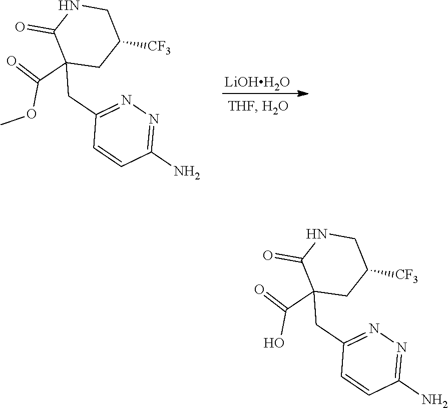 [SMILES: COC(=O)C1(Cc2ccc(N)nn2)C[C@@H](C(F)(F)F)CNC1=O ; "LiOH·H2O" ; Nc1ccc(CC2(C(=O)O)C[C@@H](C(F)(F)F)CNC2=O)nn1]

To a solution of methyl (5R)-3-((6-aminopyridazin-3-yl)methyl)-2-oxo-5-(trifluoromethyl)piperidine-3-carboxylate (4.00 g, 8.96 mmol, 1.00 eq, TFA salt) in THF (20.0 mL) and water (20.0 mL) was added LiOH·H₂O (1.13 g, 26.9 mmol, 3.00 eq). The reaction mixture was stirred at RT for 1 h. The reaction mixture was diluted with water, and a 1M aqueous solution of HCl was added to adjust the pH to 7. The reaction mixture was concentrated under reduced pressure to afford (5R)-3-((6-aminopyridazin-3-yl)methyl)-2-oxo-5-(trifluoromethyl)piperidine-3-carboxylic acid (2.80 g, 8.80 mmol, 98% yield) as a white solid.

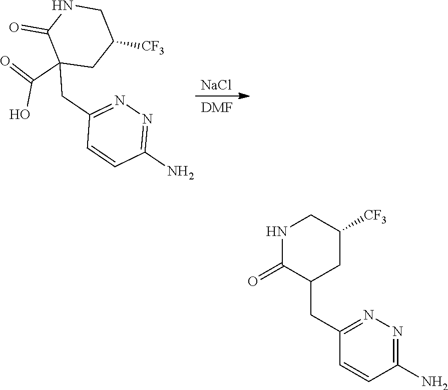

To a solution of (5R)-3-((6-aminopyridazin-3-yl)methyl)-2-oxo-5-(trifluoromethyl)piperidine-3-carboxylic acid (2.80 g, 8.80 mmol, 1.00 eq) in DMF (30.0 mL) was added NaCl (1.03 g, 17.6 mmol, 2.00 eq). The reaction mixture was stirred at 120° C. for 1 h. The reaction mixture was filtered and concentrated under reduced pressure to give a residue, which was purified by prep-HPLC (Kromasil Eternity XT (250 mm×80 mm, 10 mm); mobile phase: [water (NH₄OH)-ACN]; B %: 0%-30%) to afford (5R)-3-((6-aminopyridazin-3-yl)methyl)-5-(trifluoromethyl)piperidin-2-one. The title compound was isolated as the first eluting, single stereoisomer by chiral SFC purification (Daicel Chiralpak AD (250 mm×30 mm, 10 mm); mobile phase: [0.1% NH₃H₂O MeOH]; B %: 3 5%-3 5%); (0.70 g, 2.55 mmol, 29% yield) and was obtained as a white solid. LCMS [M+H]⁺=275.1 m/z.

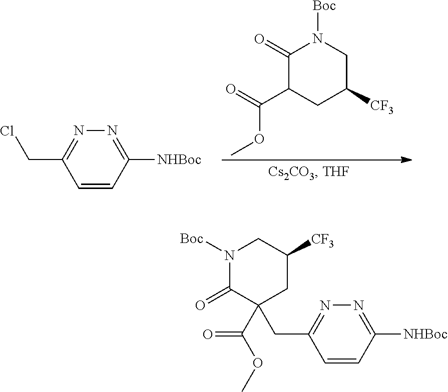 [SMILES: CC(C)(C)OC(=O)Nc1ccc(CCl)nn1 ; COC(=O)C1C[C@H](C(F)(F)F)CN(C(=O)OC(C)(C)C)C1=O ; COC(=O)C1(Cc2ccc(NC(=O)OC(C)(C)C)nn2)C[C@H](C(F)(F)F)CN(C(=O)OC(C)(C)C)C1=O]

To a solution of 1-(tert-butyl) 3-methyl (5S)-2-oxo-5-(trifluoromethyl)piperidine-1,3-dicarboxylate (23329, 1.30 g, 4.00 mmol, 1.00 eq) and tert-butyl (6-(chloromethyl)pyridazin-3-yl)carbamate (1.17 g, 4.80 mmol, 1.2 eq) in THF (10 mL) was added Cs₂CO₃ (3.91 g, 11.9 mmol, 3.00 eq). The reaction mixture was stirred at 70° C. for 2 h. The reaction mixture was diluted with water and extracted with EtOAc, dried over anhydrous Na₂SO₄, filtered and concentrated under reduced pressure to give a residue, which was purified by column chromatography (SiO₂, PE:EtOAc) to afford 1-(tert-butyl)₃-methyl (5S)-3-((6-((tert-butoxycarbonyl)amino)pyridazin-3-yl)methyl)-2-oxo-5-(trifluoromethyl)piperidine-1,3-dicarboxylate (1.30 g, 2.44 mmol, 61% yield) as a white solid. LCMS [M+H]⁺=533.2 m/z.

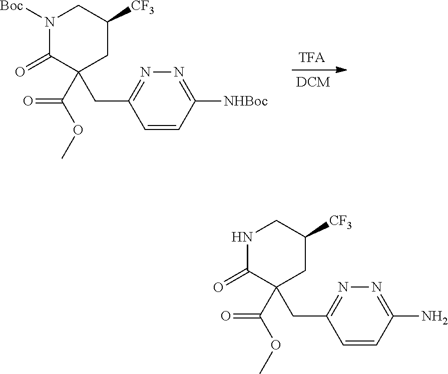 [SMILES: COC(=O)C1(Cc2ccc(NC(=O)OC(C)(C)C)nn2)C[C@H](C(F)(F)F)CN(C(=O)OC(C)(C)C)C1=O ; COC(=O)C1(Cc2ccc(N)nn2)C[C@H](C(F)(F)F)CNC1=O]

To a solution of 1-(tert-butyl) 3-methyl (5S)-3-((6-((tert-butoxycarbonyl)amino)pyridazin-3-yl)methyl)-2-oxo-5-(trifluoromethyl)piperidine-1,3-dicarboxylate (1.30 g, 2.44 mmol, 1.00 eq) in DCM (6.0 mL) was added TFA (5.57 g, 48.8 mmol, 3.61 mL, 20.0 eq) at 0° C. The reaction mixture was stirred at RT for 1 h. The reaction mixture was concentrated under reduced pressure to afford methyl (5S)-3-((6-aminopyridazin-3-yl)methyl)-2-oxo-5-(trifluoromethyl)piperidine-3-carboxylate (0.00 g, 2.24 mmol, 92% yield, TFA salt) as a white solid. LCMS [M+H]⁺=333.3 m/z.

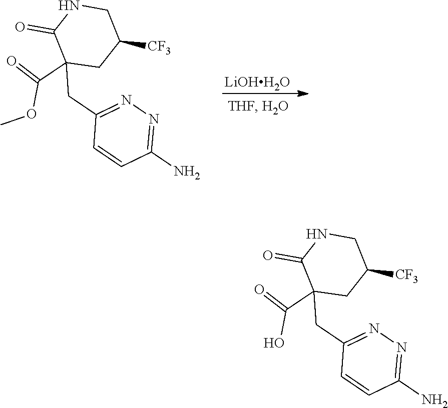 [SMILES: COC(=O)C1(Cc2ccc(N)nn2)C[C@H](C(F)(F)F)CNC1=O ; "LiOH·H2O" ; Nc1ccc(CC2(C(=O)O)C[C@H](C(F)(F)F)CNC2=O)nn1]

To a solution of methyl (5S)-3-((6-aminopyridazin-3-yl)methyl)-2-oxo-5-(trifluoromethyl)piperidine-3-carboxylate (1.00 g, 2.24 mmol, 1.0 eq, TFA salt) in THF (5.0 mL) and water (1.0 mL) was added LiOH·H₂O (376 mg, 8.96 mmol, 4.00 eq). The reaction mixture was stirred at RT for 1 h. The reaction mixture was diluted with water, and a 1M aqueous solution of HCl was added to adjust the pH to 7. The reaction mixture was concentrated under reduced pressure to afford (5S)-3-((6-aminopyridazin-3-yl)methyl)-2-oxo-5-(trifluoromethyl)piperidine-3-carboxylic acid (700 mg, 2.20 mmol, 98% yield) as a white solid.

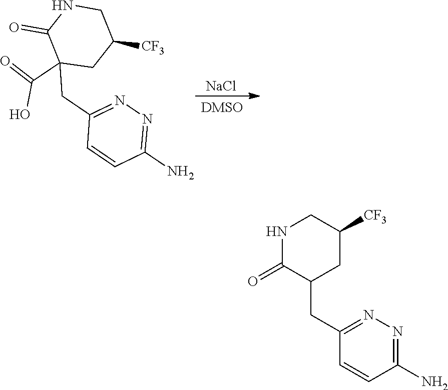

To a solution of (5S)-3-((6-aminopyridazin-3-yl)methyl)-2-oxo-5-(trifluoromethyl)piperidine-3-carboxylic acid (700 mg, 2.20 mmol, 1.00 eq) in DMSO (4.00 mL) was added NaCl (642 mg, 11.0 mmol, 5.00 eq). The reaction mixture was stirred at 100° C. for 1 h. The reaction mixture was filtered and concentrated under reduced pressure to give a residue, which was purified by prep-HPLC (neutral condition; Waters Xbridge C18 150 mm×50 mm, 10 μm; mobile phase: [water (NH₄HCO₃)-ACN]; B %: 6%-36%) to afford (5S)-3-((6-aminopyridazin-3-yl)methyl)-5-(trifluoromethyl)piperidin-2-one. The title compound, Intermediate 9, was isolated as the first eluting, single stereoisomer by chiral SFC purification (Daicel Chiralpak AD (250 mm×30 mm, 10 mm); mobile phase: [0.1% NH₃H₂O in IPA]; B %: 30%-30%); (140 mg, 510 μmol, 23% yield) and was obtained as a white solid. ¹H NMR (400 MHz, DMSO-d₆) δ 7.70 (s, 1H), 7.16 (d, J=8.8 Hz, 1H), 6.71 (d, J=9.0 Hz, 1H), 6.17 (s, 2H), 3.32-3.28 (m, 1H), 3.24-3.07 (m, 2H), 3.01-2.87 (m, 1H), 2.83-2.69 (m, 2H), 1.87-1.64 (m, 2H). LCMS [M+H]⁺=275.1 m/z.

### Example 89: Preparation of Intermediate 10

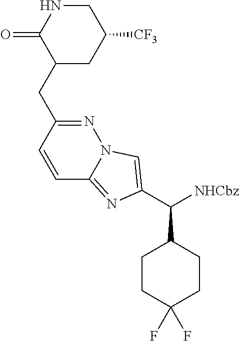 [SMILES: O=C(N[C@H](c1cn2nc(CC3C[C@@H](C(F)(F)F)CNC3=O)ccc2n1)C1CCC(F)(F)CC1)OCc1ccccc1]

Benzyl ((1S)-(4,4-difluorocyclohexyl)(6-(((5R)-2-oxo-5-(trifluoromethyl)piperidin-3-yl)methyl)imidazo[1,2-b]pyridazin-2-yl)methyl)carbamate was synthesized following the General Procedure 1, using (5R)-3-((6-aminopyridazin-3-yl)methyl)-5-(trifluoromethyl)piperidin-2-one (1.00 eq) with benzyl (S)-(3-bromo-1-(4,4-difluorocyclohexyl)-2-oxopropyl)carbamate (425 mg, 1.05 mmol, 1.20 eq), trimethyl borate (455 mg, 4.38 mmol, 494 L, 5.00 eq), DIEA (566 mg, 4.38 mmol, 762 μL, 5.00 eq) and was purified by prep-TLC (SiO₂, DCM:MeOH) to afford Intermediate 10, benzyl ((1S)-(4,4-difluorocyclohexyl)(6-(((5R)-2-oxo-5-(trifluoromethyl)piperidin-3-yl)methyl)imidazo[1,2-b]pyridazin-2-yl)methyl)carbamate, (350 mg, 604 μmol, 69% yield) as a yellow solid. LCMS [M+H]⁺=580.4 m/z.

### Example 90: Preparation of Intermediate 11

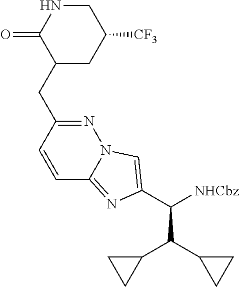 [SMILES: O=C(N[C@H](c1cn2nc(CC3C[C@@H](C(F)(F)F)CNC3=O)ccc2n1)C(C1CC1)C1CC1)OCc1ccccc1]

Benzyl ((1S)-2,2-dicyclopropyl-1-(6-(((5R)-2-oxo-5-(trifluoromethyl)piperidin-3-yl)methyl)imidazo[1,2-b]pyridazin-2-yl)ethyl)carbamate was synthesized following the General Procedure 1, using (5R)-3-((6-aminopyridazin-3-yl)methyl)-5-(trifluoromethyl)piperidin-2-one (1.00 eq) with benzyl (S)-(4-bromo-1,1-dicyclopropyl-3-oxobutan-2-yl)carbamate (665 mg, 1.75 mmol, 1.20 eq), trimethyl borate (757 mg, 7.29 mmol, 823 μL, 5.00 eq), DIEA (942 mg, 7.29 mmol, 1.27 mL, 5.00 eq) and was purified by prep-TLC (SiO₂, DCM:MeOH) to afford Intermediate 11, benzyl ((IS)-2,2-dicyclopropyl-1-(6-(((5R)-2-oxo-5-(trifluoromethyl)piperidin-3-yl)methyl)imidazo[1,2-b]pyridazin-2-yl)ethyl)carbamate, (750 mg, 1.33 mmol, 91% yield) as a yellow solid. ¹H NMR (400 MHz, MeOD) δ 7.96 (s, 1H), 7.84 (d, J=9.6 Hz, 1H), 7.27-7.42 (m, 5H), 7.18 (d, J=9.2 Hz, 1H), 5.12-5.19 (m, 2H), 3.43-3.53 (m, 2H), 3.37 (d, J=10.4 Hz, 1H), 2.87-3.12 (m, 3H), 2.14-2.22 (m, 1H), 1.64-1.82 (m, 1H), 0.67-0.83 (m, 3H), 0.11-0.50 (m, 7H), (−0.18)-(−0.20) (m, 1H). LCMS [M+H]⁺=556.3 m/z.

### Example 91: Preparation of Intermediate 12

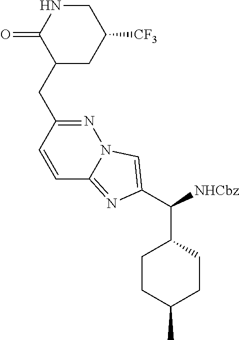 [SMILES: C[C@H]1CC[C@H]([C@H](NC(=O)OCc2ccccc2)c2cn3nc(CC4C[C@@H](C(F)(F)F)CNC4=O)ccc3n2)CC1]

Benzyl ((1S)-((1R.4S)-4-methylcyclohexyl)(6-(((5R)-2-oxo-5-(trifluoromethyl)piperidin-3-yl)methyl)imidazo[1,2-b]pyridazin-2-yl)methyl)carbamate was synthesized following the General Procedure 1, using (5R)-3-((6-aminopyridazin-3-yl)methyl)-5-(trifluoromethyl)piperidin-2-one (1.00 eq) with benzyl ((S)-3-bromo-1-((1R,4S)-4-methylcyclohexyl)-2-oxopropyl)carbamate (669 mg, 1.75 mmol, 1.20 eq), trimethyl borate (758 mg, 7.29 mmol, 824 μL, 5.00 eq), DIEA (943 mg, 7.29 mmol, 1.27 mL, 5.00 eq) and was purified by prep-TLC (SiO₂, DCM:MeOH) to afford Intermediate 12, benzyl ((1S)-((1R.4S)-4-methylcyclohexyl)(6-(((5R)-2-oxo-5-(trifluoromethyl)piperidin-3-yl)methyl)imidazo[1,2-b]pyridazin-2-yl)methyl)carbamate, (500 mg, 0.85 mmol, 58% yield) as a yellow solid. ¹H NMR (400 MHz, MeOD) δ 7.90 (s, 1H), 7.83 (d, J=9.2 Hz, 1H), 7.25-7.39 (m, 5H), 7.16 (d, J=9.2 Hz, 1H), 5.03-5.11 (m, 2H), 4.66-4.70 (m, 1H), 3.41-3.51 (m, 2H), 3.35-3.39 (m, 1H), 2.99-3.10 (m, 2H), 2.88-2.91 (m, 1H), 2.15-2.18 (m, 1H), 1.65-1.86 (m, 5H), 1.49-1.53 (m, 1H), 1.25-1.29 (m, 1H), 1.04-1.14 (m, 2H), 0.89-0.95 (m, 2H), 0.86 (d, J=6.8 Hz, 3H). LCMS [M+H]⁺=558.4 m z.

### Example 92: Preparation of Intermediates 13 and 14

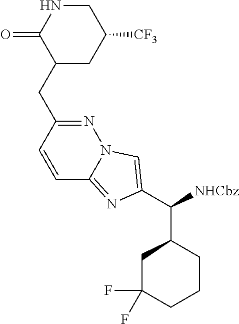 [SMILES: O=C(N[C@H](c1cn2nc(CC3C[C@@H](C(F)(F)F)CNC3=O)ccc2n1)[C@H]1CCCC(F)(F)C1)OCc1ccccc1]

Benzyl ((IS)—((S)-3,3-difluorocyclohexyl)(6-(((5R)-2-oxo-5-(trifluoromethyl)piperidin-3-yl)methyl)imidazo[1,2-b]pyridazin-2-yl)methyl)carbamate was synthesized following the General Procedure 1, using (5R)-3-((6-aminopyridazin-3-yl)methyl)-5-(trifluoromethyl)piperidin-2-one (1.00 eq) with benzyl ((S)-3-bromo-1-((S)-3,3-difluorocyclohexyl)-2-oxopropyl)carbamate (0.480 g, 1.19 mmol, 1.00 eq), trimethyl borate (617 mg, 5.94 mmol, 671 μL, 5.00 eq), DIEA (767 mg, 5.94 mmol, 1.03 mL, 5.00 eq) and was purified by prep-TLC (SiO₂, DCM:MeOH) to afford Intermediate 13, benzyl ((IS)—((S)-3,3-difluorocyclohexyl)(6-(((5R)-2-oxo-5-(trifluoromethyl)piperidin-3-yl)methyl)imidazo[1,2-b]pyridazin-2-yl)methyl)carbamate, (103 mg, 178 μmol, 15% yield) as a yellow solid. LCMS [M+H]⁺=580.4 m/z.

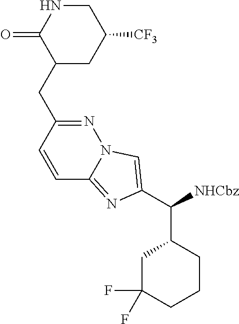 [SMILES: O=C(N[C@H](c1cn2nc(CC3C[C@@H](C(F)(F)F)CNC3=O)ccc2n1)[C@@H]1CCCC(F)(F)C1)OCc1ccccc1]

Benzyl ((IS)—((R)-3,3-difluorocyclohexyl)(6-(((5R)-2-oxo-5-(trifluoromethyl)piperidin-3-yl)methyl)imidazo[1,2-b]pyridazin-2-yl)methyl)carbamate was synthesized following the General Procedure 1, using (5R)-3-((6-aminopyridazin-3-yl)methyl)-5-(trifluoromethyl)piperidin-2-one (1.00 eq) with benzyl ((S)-3-bromo-1-((R)-3,3-difluorocyclohexyl)-2-oxopropyl)carbamate (350 mg, 866 μmol, 1.00 eq), trimethyl borate (450 mg, 4.33 mmol, 489 μL, 5.00 eq), DIEA (560 mg, 4.33 mmol, 754 μL, 5.00 eq) and was purified by prep-TLC (SiO₂, DCM:MeOH) to afford Intermediate 14, benzyl ((1S)—((R)-3,3-difluorocyclohexyl)(6-(((5R)-2-oxo-5-(trifluoromethyl)piperidin-3-yl)methyl)imidazo[1,2-b]pyridazin-2-yl)methyl)carbamate, (70.0 mg, 121 μmol, 14% yield) as a yellow solid. LCMS [M+H]⁺=580.3 m/z.

### Example 93: Preparation of Intermediate 15

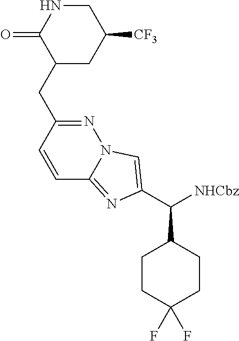 [SMILES: O=C(N[C@H](c1cn2nc(CC3C[C@H](C(F)(F)F)CNC3=O)ccc2n1)C1CCC(F)(F)CC1)OCc1ccccc1]

Benzyl ((1S)-(4,4-difluorocyclohexyl)(6-(((5S)-2-oxo-5-(trifluoromethyl)piperidin-3-yl)methyl)imidazo[1,2-b]pyridazin-2-yl)methyl)carbamate was synthesized following the General Procedure 1, using (5S)-3-((6-aminopyridazin-3-yl)methyl)-5-(trifluoromethyl)piperidin-2-one (1.00 eq) with benzyl (S)-(3-bromo-1-(4,4-difluorocyclohexyl)-2-oxopropyl)carbamate (106 mg, 262 μmol, 1.20 eq), trimethyl borate (113 mg, 1.09 mmol, 123 μL, 5.00 eq), DIEA (141 mg, 1.09 mmol, 190 μL, 5.00 eq) and was purified by prep-TLC (SiO₂, DCM:MeOH) to afford Intermediate 15, benzyl ((1S)-(4,4-difluorocyclohexyl)(6-(((5S)-2-oxo-5-(trifluoromethyl)piperidin-3-yl)methyl)imidazo[1,2-b]pyridazin-2-yl)methyl)carbamate, (100 mg, 172 μmol, 79% yield) as a white solid. LCMS [M+H]⁺=580.1 m/z.

### Example 94: Preparation of Intermediates 16 and 17

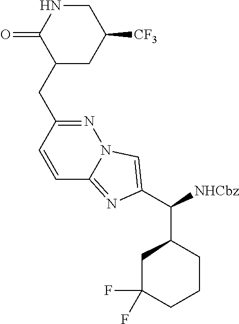 [SMILES: O=C(N[C@H](c1cn2nc(CC3C[C@H](C(F)(F)F)CNC3=O)ccc2n1)[C@H]1CCCC(F)(F)C1)OCc1ccccc1]

Benzyl ((IS)—((S)-3,3-difluorocyclohexyl)(6-(((5S)-2-oxo-5-(trifluoromethyl)piperidin-3-yl)methyl)imidazo[1,2-b]pyridazin-2-yl)methyl)carbamate was synthesized following the General Procedure 1, using (5S)-3-((6-aminopyridazin-3-yl)methyl)-5-(trifluoromethyl)piperidin-2-one (1.00 eq) with benzyl ((S)-3-bromo-1-((S)-3,3-difluorocyclohexyl)-2-oxopropyl)carbamate (250 mg, 618 μmol, 1.00 eq), trimethyl borate (321 mg, 3.09 mmol, 618 μmol, 349 μL, 5.00 eq), DIEA (399 mg, 3.09 mmol, 538 μL, 107 μL, 5.00 eq) and was purified by prep-TLC (SiO₂, DCM:MeOH) to afford Intermediate 16, benzyl ((IS)—((S)-3,3-difluorocyclohexyl)(6-(((5S)-2-oxo-5-(trifluoromethyl)piperidin-3-yl)methyl)imidazo[1,2-b]pyridazin-2-yl)methyl)carbamate, (70.0 mg, 120 μmol, 20% yield) as a yellow solid. LCMS [M+H]⁺=580.4 m/z.

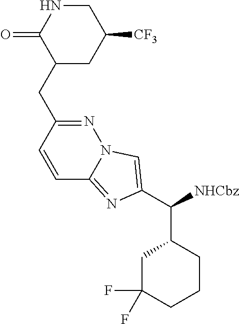 [SMILES: O=C(N[C@H](c1cn2nc(CC3C[C@H](C(F)(F)F)CNC3=O)ccc2n1)[C@@H]1CCCC(F)(F)C1)OCc1ccccc1]

Benzyl ((IS)—((R)-3,3-difluorocyclohexyl)(6-(((5S)-2-oxo-5-(trifluoromethyl)piperidin-3-yl)methyl)imidazo[1,2-b]pyridazin-2-yl)methyl)carbamate was synthesized following the General Procedure 1, using (5S)-3-((6-aminopyridazin-3-yl)methyl)-5-(trifluoromethyl)piperidin-2-one (1.00 eq) with benzyl ((S)-3-bromo-1-((R)-3,3-difluorocyclohexyl)-2-oxopropyl)carbamate (260 mg, 643 μmol, 1.00 eq), trimethyl borate (334 mg, 3.22 mmol, 363 μL, 5.00 eq), DIEA (665 mg, 5.15 mmol, 896 μL, 8.00 eq) and was purified by prep-HPLC (Phenomenexluna C18 150×25 mm, 10 μm; mobile phase: [water (FA)-ACN]; B %: 40%-70%) to afford Intermediate 17, benzyl ((1S)—((R)-3,3-difluorocyclohexyl)(6-(((5S)-2-oxo-5-(trifluoromethyl)piperidin-3-yl)methyl)imidazo[1,2-b]pyridazin-2-yl)methyl)carbamate, (115.0 mg, 199 μmol, 31% yield) as a yellow gum. LCMS [M+H]⁺=580.2 m/z.

### Example 95: Preparation of Intermediate 18

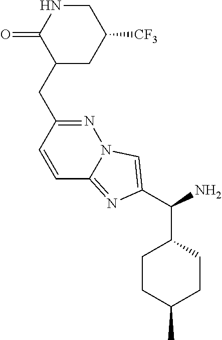 [SMILES: C[C@H]1CC[C@H]([C@H](N)c2cn3nc(CC4C[C@@H](C(F)(F)F)CNC4=O)ccc3n2)CC1]

(5R)-3-((2-((S)-amino((1R.4S)-4-methylcyclohexyl)methyl)imidazo[1,2-b]pyridazin-6-yl)methyl)-5-(trifluoromethyl)piperidin-2-one was synthesized following the General Procedure 2, using benzyl ((1S)-((1R.4S)-4-methylcyclohexyl)(6-(((5R)-2-oxo-5-(trifluoromethyl)piperidin-3-yl)methyl)imidazo[1,2-b]pyridazin-2-yl)methyl)carbamate (1.50 g, 2.69 mmol, 1.00 eq) with iodotrimethylsilane (1.61 g, 8.07 mmol, 1.10 mL, 3.00 eq) to afford Intermediate 18, (5R)-3-((2-((S)-amino((1R.4S)-4-methylcyclohexyl)methyl)imidazo[1,2-b]pyridazin-6-yl)methyl)-5-(trifluoromethyl)piperidin-2-one, (1.10 g, 2.60 mmol, 97% yield) as a yellow solid. LCMS [M+H]⁺=424.3 m/z.

### Example 96: Preparation of Intermediates 19 and 20

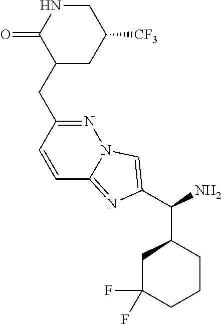 [SMILES: N[C@H](c1cn2nc(CC3C[C@@H](C(F)(F)F)CNC3=O)ccc2n1)[C@H]1CCCC(F)(F)C1]

(5R)-3-((2-((S)-amino((S)-3,3-difluorocyclohexyl)methyl)imidazo[1,2-b]pyridazin-6-yl)methyl)-5-(trifluoromethyl)piperidin-2-one was synthesized following the General Procedure 2, using benzyl ((IS)—((S)-3,3-difluorocyclohexyl)(6-(((5R)-2-oxo-5-(trifluoromethyl)piperidin-3-yl)methyl)imidazo[1,2-b]pyridazin-2-yl)methyl)carbamate (103 mg, 178 μmol, 1.00 eq) with iodotrimethylsilane (107 mg, 533 μmol, 72.6 μL, 3.00 eq) to afford Intermediate 19, (5R)-3-((2-((S)-amino((S)-3,3-difluorocyclohexyl)methyl)imidazo[1,2-b]pyridazin-6-yl)methyl)-5-(trifluoromethyl)piperidin-2-one, (60.0 mg, 135 μmol, 76% yield) as a yellow solid. LCMS [M+H]⁺=446.2 m/z.

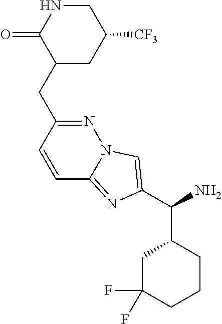 [SMILES: N[C@H](c1cn2nc(CC3C[C@@H](C(F)(F)F)CNC3=O)ccc2n1)[C@@H]1CCCC(F)(F)C1]

(5R)-3-((2-((S)-amino((R)-3,3-difluorocyclohexyl)methyl)imidazo[1,2-b]pyridazin-6-yl)methyl)-5-(trifluoromethyl)piperidin-2-one was synthesized following the General Procedure 2, using benzyl ((IS)—((R)-3,3-difluorocyclohexyl)(6-(((5R)-2-oxo-5-(trifluoromethyl)piperidin-3-yl)methyl)imidazo[1,2-b]pyridazin-2-yl)methyl)carbamate (70.0 mg, 121 μmol, 1.00 eq) with iodotrimethylsilane (72.5 mg, 362 μmol, 49.3 μL, 3.00 eq) to afford Intermediate 20, (5R)-3-((2-((S)-amino((R)-3,3-difluorocyclohexyl)methyl)imidazo[1,2-b]pyridazin-6-yl)methyl)-5-(trifluoromethyl)piperidin-2-one, (40.0 mg, 89.8 μmol, 74% yield) as a yellow solid. LCMS [M+H]⁺=446.4 m/z.

### Example 97: Preparation of Intermediate 21

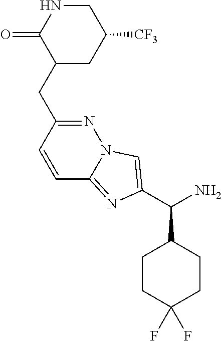 [SMILES: N[C@H](c1cn2nc(CC3C[C@@H](C(F)(F)F)CNC3=O)ccc2n1)C1CCC(F)(F)CC1]

(5R)-3-((2-((S)-amino(4,4-difluorocyclohexyl)methyl)imidazo[1,2-b]pyridazin-6-yl)methyl)-5-(trifluoromethyl)piperidin-2-one was synthesized following the General Procedure 3, using benzyl ((1S)-(4,4-difluorocyclohexyl)(6-(((5R)-2-oxo-5-(trifluoromethyl)piperidin-3-yl)methyl)imidazo[1,2-b]pyridazin-2-yl)methyl)carbamate (350 mg, 604 μmol, 1.00 eq) with Pd/C (50.0 mg, 10% purity) to afford Intermediate 21, (5R)-3-((2-((S)-amino(4,4-difluorocyclohexyl)methyl)imidazo[1,2-b]pyridazin-6-yl)methyl)-5-(trifluoromethyl)piperidin-2-one, (250 mg, 561 μmol, 93% yield) as a yellow solid. LCMS [M+H]⁺=446.2 m/z.

### Example 98: Preparation of Intermediate 22

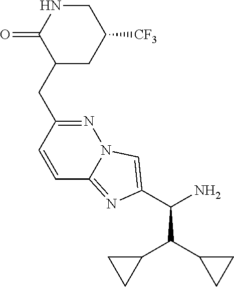 [SMILES: N[C@H](c1cn2nc(CC3C[C@@H](C(F)(F)F)CNC3=O)ccc2n1)C(C1CC1)C1CC1]

(5R)-3-((2-((S)-1-amino-2,2-dicyclopropylethyl)imidazo[1,2-b]pyridazin-6-yl)methyl)-5-(trifluoromethyl)piperidin-2-one was synthesized following the General Procedure 3, using benzyl ((1S)-2,2-dicyclopropyl-1-(6-(((5R)-2-oxo-5-(trifluoromethyl)piperidin-3-yl)methyl)imidazo[1,2-b]pyridazin-2-yl)ethyl)carbamate (180 mg, 323 μmol, 1.00 eq) with Pd/C (20 mg, 10% purity) to afford Intermediate 22, (5R)-3-((2-((S)-1-amino-2,2-dicyclopropylethyl)imidazo[1,2-b]pyridazin-6-yl)methyl)-5-(trifluoromethyl)piperidin-2-one, (90.0 mg, 213 μmol, 66% yield) as a yellow oil. LCMS [M+H]⁺=422.3 m/z.

### Example 98: Preparation of Intermediate 23

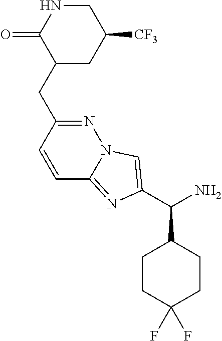 [SMILES: N[C@H](c1cn2nc(CC3C[C@H](C(F)(F)F)CNC3=O)ccc2n1)C1CCC(F)(F)CC1]

(5S)-3-((2-((S)-amino(4,4-difluorocyclohexyl)methyl)imidazo[1,2-b]pyridazin-6-yl)methyl)-5-(trifluoromethyl)piperidin-2-one was synthesized following the General Procedure 3, using benzyl ((1S)-(4,4-difluorocyclohexyl)(6-(((5S)-2-oxo-5-(trifluoromethyl)piperidin-3-yl)methyl)imidazo[1,2-b]pyridazin-2-yl)methyl)carbamate (100 mg, 172 μmol, 1.00 eq) with Pd/C (150.0 mg, 10% purity) to afford Intermediate 23, (5S)-3-((2-((S)-amino(4,4-difluorocyclohexyl)methyl)imidazo[1,2-b]pyridazin-6-yl)methyl)-5-(trifluoromethyl)piperidin-2-one (70.0 mg, 157 μmol, 91% yield) as a white solid. LCMS [M+H]⁺=446.3 m/z.

### Example 99: Preparation of Compound 95

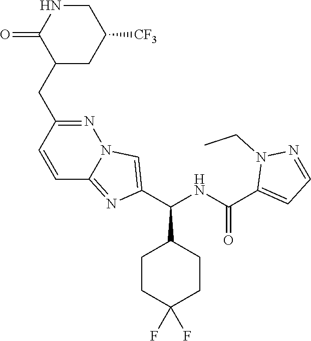 [SMILES: CCn1nccc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (80.0 mg, 179 μmol, 1.00 eq), 1-ethyl-1H-pyrazole-5-carboxylic acid (37.7 mg, 269 μmol, 1.50 eq) and was purified by prep-HPLC (neutral condition; Waters Xbridge 150 mm×25 mm, 5 um; mobile phase: [water (NH₄HCO₃)-ACN]; B %: 35%-65%) to afford Compound 95, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, (27.73 mg, 48.8 μmol, 27% yield) as a white solid. LCMS [M+H]⁺=568.4 m/z.

### Example 100: Preparation of Compound 96

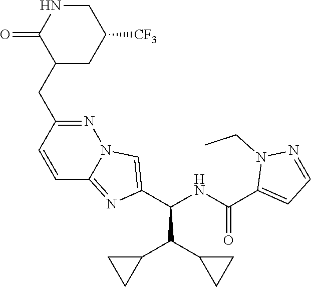 [SMILES: CCn1nccc1C(=O)N[C@H](c1cn2nc(CC3C[C@@H](C(F)(F)F)CNC3=O)ccc2n1)C(C1CC1)C1CC1]

N-((1S)-2,2-dicyclopropyl-1-(6-(((5R)-2-oxo-5-(trifluoromethyl)piperidin-3-yl)methyl)imidazo[1,2-b]pyridazin-2-yl)ethyl)-1-ethyl-1H-pyrazole-5-carboxamide was synthesized following the General Procedure 4 (GP4) with (5R)-3-((2-((S)-1-amino-2,2-dicyclopropylethyl)imidazo[1,2-b]pyridazin-6-yl)methyl)-5-(trifluoromethyl)piperidin-2-one (90.0 mg, 213 μmol, 1.00 eq), 1-ethyl-1H-pyrazole-5-carboxylic acid (44.8 mg, 320 μmol, 1.50 eq) and was purified by prep-TLC (SiO₂, DCM:MeOH) to afford Compound 96, N-((1S)-2,2-dicyclopropyl-1-(6-(((5R)-2-oxo-5-(trifluoromethyl)piperidin-3-yl)methyl)imidazo[1,2-b]pyridazin-2-yl)ethyl)-1-ethyl-1H-pyrazole-5-carboxamide, (48.45 mg, 88.2 μmol, 41% yield) as a light yellow solid. LCMS [M+H]⁺=544.3 m/z.

### Example 101: Preparation of Compound 97

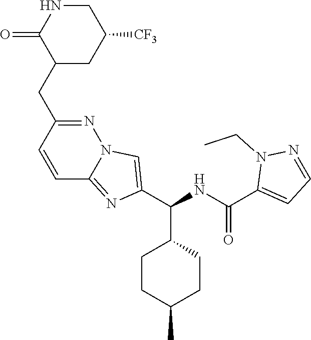 [SMILES: CCn1nccc1C(=O)N[C@H](c1cn2nc(CC3C[C@@H](C(F)(F)F)CNC3=O)ccc2n1)[C@H]1CC[C@H](C)CC1]

1-Ethyl-N-((1S)-((1R.4S)-4-methylcyclohexyl)(6-(((5R)-2-oxo-5-(trifluoromethyl)piperidin-3-yl)methyl)imidazo[1,2-b]pyridazin-2-yl)methyl)-1H-pyrazole-5-carboxamide was synthesized following the General Procedure 4 with (5R)-3-((2-((S)-amino((1R.4S)-4-methylcyclohexyl)methyl)imidazo[1,2-b]pyridazin-6-yl)methyl)-5-(trifluoromethyl)piperidin-2-one (1.10 g, 2.60 mmol, 1.00 eq), 1-ethyl-1H-pyrazole-5-carboxylic acid (546 mg, 3.90 mmol, 1.50 eq) and was purified by column chromatography (SiO₂, DCM/MeOH) to afford Compound 97, 1-ethyl-N-((1S)-((1R.4S)-4-methylcyclohexyl)(6-(((5R)-2-oxo-5-(trifluoromethyl)piperidin-3-yl)methyl)imidazo[1,2-b]pyridazin-2-yl)methyl)-1H-pyrazole-5-carboxamide, (1.31 g, 2.40 mmol, 92% yield) as a yellow solid. LCMS [M+H]⁺=546.4 m/z.

### Example 102: Preparation of Compounds 98 and 99

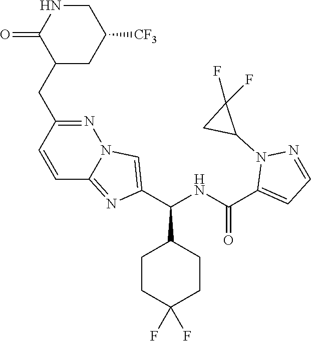 [SMILES: O=C(N[C@H](c1cn2nc(CC3C[C@@H](C(F)(F)F)CNC3=O)ccc2n1)C1CCC(F)(F)CC1)c1ccnn1C1CC1(F)F]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-(2,2-difluorocyclopropyl)-1H-pyrazole-5-carboxamide were synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (75.0 mg, 169 μmol, 1.00 eq), 1-(2,2-difluorocyclopropyl)-1H-pyrazole-5-carboxylic acid (48.0 mg, 253 μmol, 1.50 eq) and was purified by prep-TLC (SiO₂, DCM:MeOH) to afford N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-(2,2-difluorocyclopropyl)-1H-pyrazole-5-carboxamide (65.0 mg, 106 μmol, 63% yield) as a white solid. LCMS [M+H]⁺=616.3 m/z.

The title compound Compound 98 was isolated as the first eluting, single stereoisomer by chiral SFC purification (Daicel Chiralpak AD (250 mm×30 mm, 10 μm); mobile phase: [NH₃H₂O in MeOH]; B %: 35%-35%); (25.72 mg, 37.73 μmol, 3 6% yield) and was obtained as a white solid. LCMS [M+H]⁺=616.3 m/z.

The title compound Compound 99 was isolated as the second eluting, single stereoisomer by chiral SFC purification (Daicel Chiralpak AD (250 mm×30 mm, 10 μm); mobile phase: [NH₃H₂O in MeOH]; B %: 35%-35%); (23.00 mg, 36.02 μmol, 34% yield) and was obtained as a white solid. LCMS [M+H]⁺=616.3 m/z.

### Example 103: Preparation of Compound 100

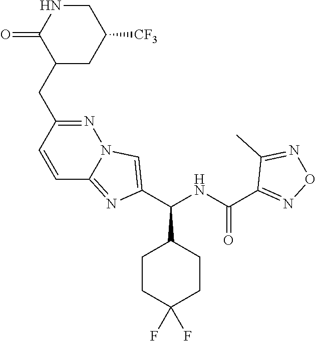 [SMILES: Cc1nonc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-methyl-1,2,5-oxadiazole-3-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (45.0 mg, 101 μmol, 1.00 eq), 4-methyl-1,2,5-oxadiazole-3-carboxylic acid (15.5 mg 121 μmol, 1.20 eq) and was purified by prep-TLC (SiO₂, DCM:MeOH) to afford Compound 100, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-methyl-1,2,5-oxadiazole-3-carboxamide, (17.30 mg, 29.37 μmol, 29% yield) as a white solid. LCMS [M+H]⁺=556.2 m/z.

### Example 104: Preparation of Compound 101

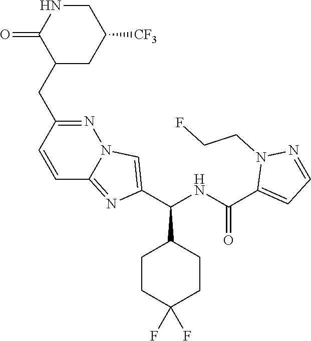 [SMILES: O=C(N[C@H](c1cn2nc(CC3C[C@@H](C(F)(F)F)CNC3=O)ccc2n1)C1CCC(F)(F)CC1)c1ccnn1CCF]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-(2-fluoroethyl)-1H-pyrazole-5-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (45.0 mg, 101 μmol, 1.00 eq), 1-(2-fluoroethyl)-1H-pyrazole-5-carboxylic acid (24.0 mg 152 μmol, 1.50 eq) and was purified by prep-TLC (SiO₂, DCM:MeOH) to afford Compound 101, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-(2-fluoroethyl)-1H-pyrazole-5-carboxamide, (10.51 mg, 17.25 μmol, 17% yield) as a white solid. LCMS [M+H]⁺=586.2 m/z.

### Example 105: Preparation of Compound 102

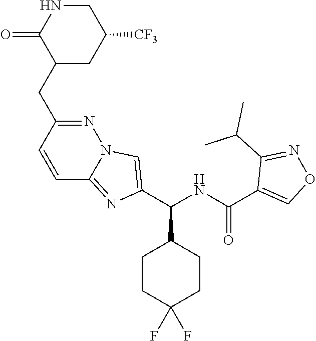 [SMILES: CC(C)c1nocc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-isopropylisoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (35.0 mg, 78.5 μmol, 1.00 eq), 3-isopropylisoxazole-4-carboxylic acid (24.3 mg, 157 μmol, 2.00 eq) and was purified by prep-HPLC (neutral condition; Waters Xbridge 150 mm×25 mm, 5 um; mobile phase: [water (NH₄HCO₃)-ACN]; B %: 40%-70%) to afford Compound 102, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-isopropylisoxazole-4-carboxamide (32.6 mg 52.1 μmol, 66% yield) as a white solid. LCMS [M+H]⁺=583.3 m/z.

### Example 106: Preparation of Compound 103

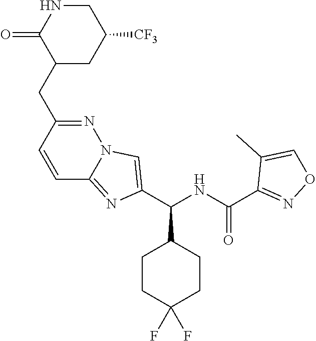 [SMILES: Cc1conc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-methylisoxazole-3-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (35.0 mg, 78.6 μmol, 1.00 eq), 4-methylisoxazole-3-carboxylic acid (11.9 mg, 94.3 μmol, 1.20 eq) and was purified by prep-HPLC (neutral condition; Waters Xbridge 150 mm×25 mm, 5 um; mobile phase: [water (NH₄HCO₃)-ACN]; B %: 36%-66%) to afford Compound 103, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-methylisoxazole-3-carboxamide, (17.6 mg, 31.2 μmol, 40% yield) as a white solid. LCMS [M+H]⁺=555.3 m/z.

### Example 107: Preparation of Compound 104

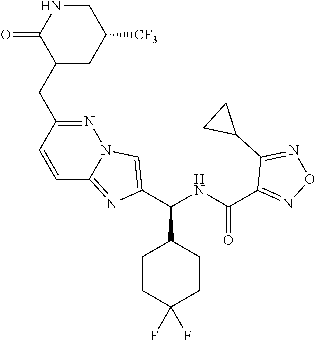 [SMILES: O=C(N[C@H](c1cn2nc(CC3C[C@@H](C(F)(F)F)CNC3=O)ccc2n1)C1CCC(F)(F)CC1)c1nonc1C1CC1]

4-Cyclopropyl-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1,2,5-oxadiazole-3-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (50.0 mg, 112 μmol, 1.00 eq), 4-cyclopropyl-1,2,5-oxadiazole-3-carboxylic acid (20.7 mg, 134 μmol, 1.20 eq) and was purified by prep-HPLC (neutral condition; Waters Xbridge 150 mm×25 mm, Sum; mobile phase: [water (NH₄HCO₃)-ACN]; B %: 43%-73%) to afford Compound 104, 4-cyclopropyl-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1,2,5-oxadiazole-3-carboxamide, (23.9 mg, 40.6 μmol, 36% yield) as a white solid. LCMS [M+H]⁺=582.3 m/z.

### Example 108: Preparation of Compound 105

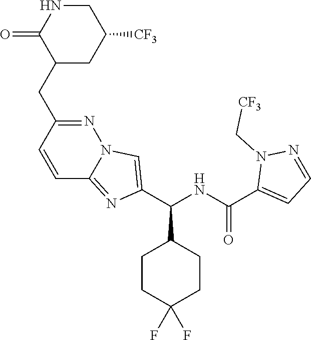 [SMILES: O=C(N[C@H](c1cn2nc(CC3C[C@@H](C(F)(F)F)CNC3=O)ccc2n1)C1CCC(F)(F)CC1)c1ccnn1CC(F)(F)F]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-(2,2,2-trifluoroethyl)-1H-pyrazole-5-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (50.0 mg, 112 μmol, 1.00 eq), 1-(2,2,2-trifluoroethyl)-1H-pyrazole-5-carboxylic acid (23.9 mg, 123 μmol, 1.10 eq) and was purified by prep-HPLC (Phenomenex luna C18 150×25 mm, 10 μm; mobile phase: [water (FA)-ACN]; B %: 42%-72%) to afford Compound 105, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-(2,2,2-trifluoroethyl)-1H-pyrazole-5-carboxamide, (43.9 mg, 69.4 mol, 62% yield) as a white solid. LCMS [M+H]⁺=622.3 m/z.

### Example 109: Preparation of Compound 106

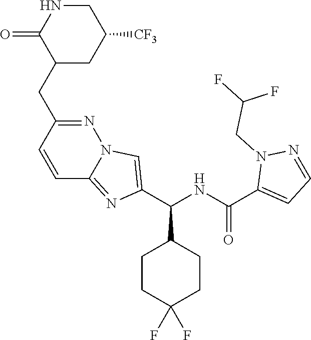 [SMILES: O=C(N[C@H](c1cn2nc(CC3C[C@@H](C(F)(F)F)CNC3=O)ccc2n1)C1CCC(F)(F)CC1)c1ccnn1CC(F)F]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-(2,2-difluoroethyl)-1H-pyrazole-5-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (50.0 mg, 112 μmol, 1.00 eq), 1-(2,2-difluoroethyl)-1H-pyrazole-5-carboxylic acid (23.7 mg, 134 μmol, 1.20 eq) and was purified by prep-HPLC (Phenomenex luna C18 150×25 mm, 10 μm; mobile phase: [water (FA)-ACN]; B %: 38%-68%) to afford Compound 106, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-(2,2-difluoroethyl)-1H-pyrazole-5-carboxamide, (54.4 mg, 89.1 mol, 79% yield) as a white solid. LCMS [M+H]⁺=604.2 m/z.

### Example 110: Preparation of Compound 107

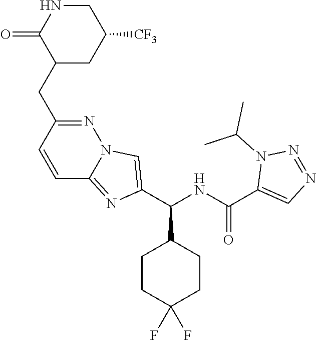 [SMILES: CC(C)n1nncc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-isopropyl-1H-1,2,3-triazole-5-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (30.0 mg, 67.3 μmol, 1.00 eq), 1-isopropyl-1H-1,2,3-triazole-5-carboxylic acid (11.4 mg 74.0 μmol, 1.10 eq) and was purified by prep-HPLC (Waters Xbridge 150 mm×25 mm, 10 μm; mobile phase: [water (NH₄HCO₃)-ACN]; B %: 32%-62%) to afford Compound 107, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-isopropyl-1H-1,2,3-triazole-5-carboxamide (36.3 mg, 61.8 μmol, 92% yield) as a white solid. LCMS [M+H]⁺=583.2 m/z.

### Example 111: Preparation of Compound 108

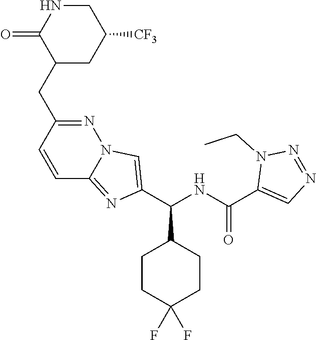 [SMILES: CCn1nncc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-ethyl-1H-1,2,3-triazole-5-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (30.0 mg, 67.3 μmol, 1.00 eq), 1-ethyl-1H-1,2,3-triazole-5-carboxylic acid (10.4 mg, 74.0 mol, 1.10 eq) and was purified by prep-TLC (SiO₂, DCM:MeOH) to afford Compound 108, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-ethyl-1H-1,2,3-triazole-5-carboxamide, (20.07 mg, 34.2 μmol, 51% yield) as a white solid. LCMS [M+H]⁺=569.2 m/z.

### Example 112: Preparation of Compound 109

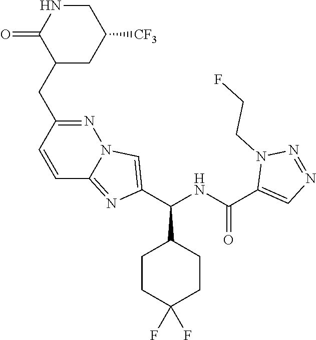 [SMILES: O=C(N[C@H](c1cn2nc(CC3C[C@@H](C(F)(F)F)CNC3=O)ccc2n1)C1CCC(F)(F)CC1)c1cnnn1CCF]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-(2-fluoroethyl)-1H-1,2,3-triazole-5-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (40.0 mg, 89.8 μmol, 1.00 eq), 1-(2-fluoroethyl)-1H-1,2,3-triazole-5-carboxylic acid (15.7 mg, 98.7 μmol, 1.10 eq) and was purified by prep-HPLC (Phenomenexluna C18 150×25 mm, 10 μm; mobile phase: [water (FA)-ACN]; B %: 28%-58%) to afford Compound 109, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-(2-fluoroethyl)-1H-1,2,3-triazole-5-carboxamide, (20.07 mg, 32.6 mol, 36% yield) as a white solid. LCMS [M+H]⁺=587.2 m/z.

### Example 113: Preparation of Compound 110

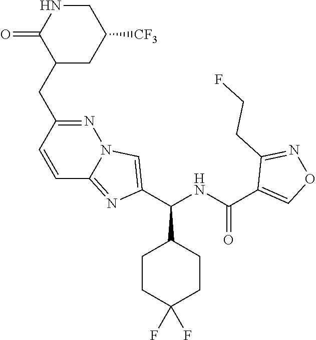 [SMILES: O=C(N[C@H](c1cn2nc(CC3C[C@@H](C(F)(F)F)CNC3=O)ccc2n1)C1CCC(F)(F)CC1)c1conc1CCF]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(2-fluoroethyl)isoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (30.0 mg, 67.3 μmol, 1.00 eq), 3-(2-fluoroethyl)isoxazole-4-carboxylic acid (12.7 mg, 74.0 mol, 1.10 eq) and was purified by prep-HPLC (Phenomenex luna C18 150×25 mm, 10 μm; mobile phase: [water (FA)-ACN]; B %: 32%-62%) to afford Compound 110, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(2-fluoroethyl)isoxazole-4-carboxamide, (16.00 mg, 25.3 μmol, 38% yield) as a white solid. LCMS [M+H]⁺=587.2 m/z.

### Example 114: Preparation of Compound 111

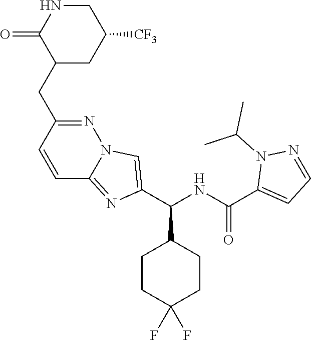 [SMILES: CC(C)n1nccc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-isopropyl-1H-pyrazole-5-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (40.0 mg, 89.8 μmol, 1.00 eq), 1-isopropyl-1H-pyrazole-5-carboxylic acid (16.6 mg, 107.8 mol, 1.20 eq) and was purified by prep-HPLC (Phenomenexluna C18 150×25 mm, 10 μm; mobile phase: [water (FA)-ACN]; B %: 37%-67%) to afford Compound 111, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-isopropyl-1H-pyrazole-5-carboxamide, (21.85 mg, 37.4 μmol, 42% yield) as a white solid. LCMS [M+H]⁺=582.3 m/z.

### Example 115: Preparation of Compound 112

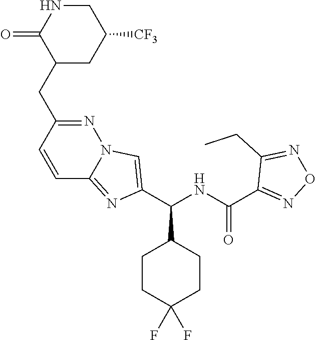 [SMILES: CCc1nonc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-ethyl-1,2,5-oxadiazole-3-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (70.0 mg, 157 μmol, 1.00 eq), 4-ethyl-1,2,5-oxadiazole-3-carboxylic acid (33.5 mg, 236 mol, 1.50 eq) and was purified by prep-TLC (SiO₂, DCM:MeOH) to afford Compound 112, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-ethyl-1,2,5-oxadiazole-3-carboxamide, (61.8 mg, 107 μmol, 68% yield) as a yellow solid. LCMS [M+H]⁺=570.3 m/z.

### Example 116: Preparation of Compound 113

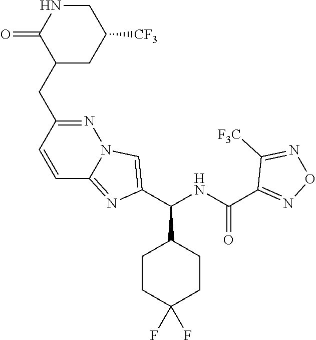 [SMILES: O=C(N[C@H](c1cn2nc(CC3C[C@@H](C(F)(F)F)CNC3=O)ccc2n1)C1CCC(F)(F)CC1)c1nonc1C(F)(F)F]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-(trifluoromethyl)-1,2,5-oxadiazole-3-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (200 mg, 449 μmol, 1.00 eq), 4-(trifluoromethyl)-1,2,5-oxadiazole-3-carboxylic acid (283 mg, 1.35 mmol, 3.00 eq) and was purified by prep-HPLC (UniSil 3-100 C18 Ultra 150×25 mm, 3 um; mobile phase: [water (FA)-ACN]; B %: 55%-75%) to afford Compound 113, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-(trifluoromethyl)-1,2,5-oxadiazole-3-carboxamide, (34 mg, 55.8 mol, 12% yield) as a white solid. LCMS [M+H]⁺=610.3 m/z.

### Example 117: Preparation of Compound 114

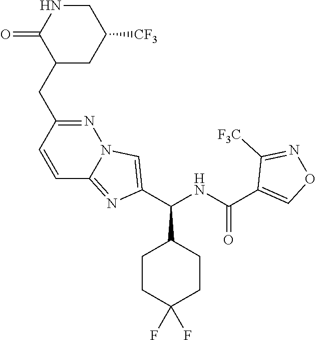 [SMILES: O=C(N[C@H](c1cn2nc(CC3C[C@@H](C(F)(F)F)CNC3=O)ccc2n1)C1CCC(F)(F)CC1)c1conc1C(F)(F)F]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(trifluoromethyl)isoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (40.0 mg, 89.8 μmol, 1.00 eq), 3-(trifluoromethyl)isoxazole-4-carboxylic acid (19.5 mg 107 μmol, 1.20 eq) and was purified by prep-HPLC (Waters Xbridge 150 mm×25 mm, 5 um; mobile phase: [water (NH₄HCO₃)-ACN]; B %: 3 8%-68%) to afford Compound 114, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(trifluoromethyl)isoxazole-4-carboxamide (10.0 mg, 16.34 μmol, 18% yield) as a white solid. LCMS [M+H]⁺=609.2 m/z.

### Example 118: Preparation of Compound 115

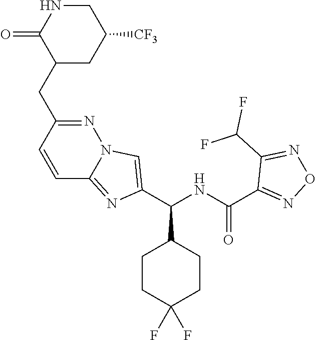 [SMILES: O=C(N[C@H](c1cn2nc(CC3C[C@@H](C(F)(F)F)CNC3=O)ccc2n1)C1CCC(F)(F)CC1)c1nonc1C(F)F]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-(difluoromethyl)-1,2,5-oxadiazole-3-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (50.0 mg, 112 μmol, 1.00 eq), 4-(difluoromethyl)-1,2,5-oxadiazole-3-carboxylic acid (27.6 mg, 168 μmol, 1.50 eq) and was purified by prep-TLC (SiO₂, DCM:MeOH) to afford Compound 115, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-(difluoromethyl)-1,2,5-oxadiazole-3-carboxamide (25.6 mg, 42.3 μmol, 38% yield) as a white solid. LCMS [M+H]⁺=592.3 m/z.

### Example 119: Preparation of Compound 116

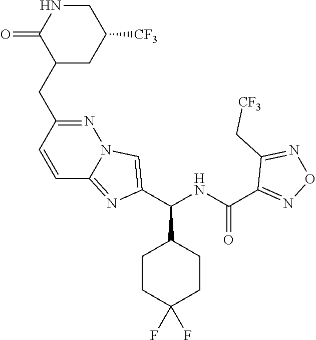 [SMILES: O=C(N[C@H](c1cn2nc(CC3C[C@@H](C(F)(F)F)CNC3=O)ccc2n1)C1CCC(F)(F)CC1)c1nonc1CC(F)(F)F]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-(2,2,2-trifluoroethyl)-1,2,5-oxadiazole-3-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (50.0 mg, 112 μmol, 1.00 eq), 4-(2,2,2-trifluoroethyl)-1,2,5-oxadiazole-3-carboxylic acid (26.4 mg, 134 μmol, 1.20 eq) and was purified by prep-HPLC (Phenomenex luna C18 150×25 mm, 10 μm; mobile phase: [water (FA)-ACN]; B %: 42%-72%) to afford Compound 116, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-(2,2,2-trifluoroethyl)-1,2,5-oxadiazole-3-carboxamide, (10.0 mg, 15.4 μmol, 14% yield) as a white solid. LCMS [M+H]⁺=624.2 m/z.

### Example 120: Preparation of Compound 117

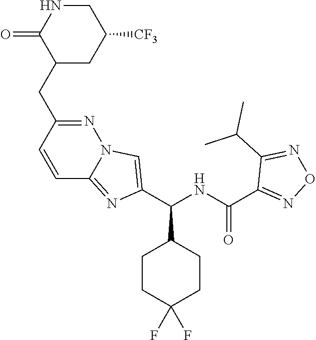 [SMILES: CC(C)c1nonc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-isopropyl-1,2,5-oxadiazole-3-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (30.0 mg, 67.3 μmol, 1.00 eq), 4-isopropyl-1,2,5-oxadiazole-3-carboxylic acid (11.5 mg, 74.0 μmol, 1.10 eq) and was purified by prep-HPLC (Phenomenexluna C18 150×25 mm, 10 μm; mobile phase: [water (FA)-ACN]; B %: 47%-77%) to afford Compound 117, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-4-isopropyl-1,2,5-oxadiazole-3-carboxamide, (29.44 mg, 49.5 μmol, 74% yield) as a white solid. LCMS [M+H]⁺=584.2 m/z.

### Example 121: Preparation of Compound 118

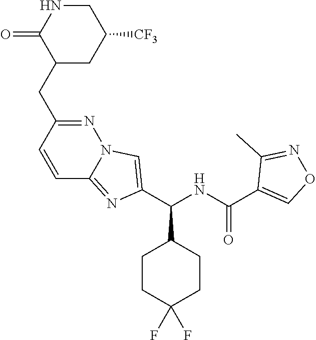 [SMILES: Cc1nocc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-methylisoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (30.0 mg, 67.3 μmol, 1.00 eq), 3-methylisoxazole-4-carboxylic acid (9.42 mg, 74.0 μmol, 1.10 eq) and was purified by prep-HPLC (Phenomenexluna C18 150×25 mm, 10 μm; mobile phase: [water (FA)-ACN]; B %: 30%-60%) to afford Compound 118, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-methylisoxazole-4-carboxamide, (26.71 mg, 47.9 μmol, 71% yield) as a light yellow solid. LCMS [M+H]⁺=555.2 m/z.

### Example 122: Preparation of Compound 119

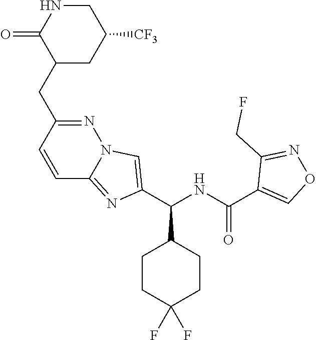 [SMILES: O=C(N[C@H](c1cn2nc(CC3C[C@@H](C(F)(F)F)CNC3=O)ccc2n1)C1CCC(F)(F)CC1)c1conc1CF]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(fluoromethyl)isoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (50.0 mg, 112 μmol, 1.00 eq), 3-(fluoromethyl)isoxazole-4-carboxylic acid (4.4 mg, 168 mol, 1.50 eq) and was purified by prep-HPLC (Waters Xbridge 150 mm×25 mm, 5 um; mobile phase: [water (NH₄HCO₃)-ACN]; B %: 35%-65%) to afford Compound 119, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(fluoromethyl)isoxazole-4-carboxamide, (36.93 mg, 60.89 μmol, 54% yield) as a white solid. LCMS [M+H]⁺=573.2 m/z.

### Example 123: Preparation of Compound 120

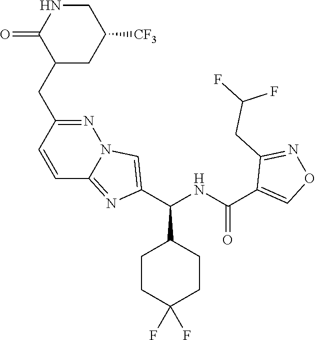 [SMILES: O=C(N[C@H](c1cn2nc(CC3C[C@@H](C(F)(F)F)CNC3=O)ccc2n1)C1CCC(F)(F)CC1)c1conc1CC(F)F]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(2,2-difluoroethyl)isoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (30.0 mg, 67.3 μmol, 1.00 eq), 3-(2,2-difluoroethyl)isoxazole-4-carboxylic acid (17.8 mg 101 μmol, 1.50 eq) and was purified by prep-TLC (SiO₂, DCM:MeOH) to afford Compound 120, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(2,2-difluoroethyl)isoxazole-4-carboxamide, (15.9 mg, 25.44 μmol, 38% yield) as a yellow solid. LCMS [M+H]⁺=605.4 m/z.

### Example 124: Preparation of Compound 121

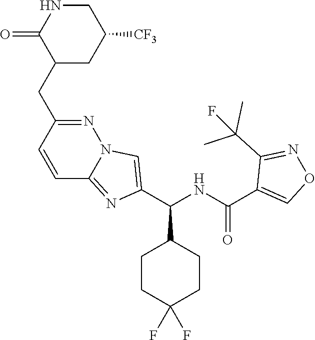 [SMILES: CC(C)(F)c1nocc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(2-fluoropropan-2-yl)isoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (30.0 mg, 67.3 μmol, 1.00 eq), 3-(2-fluoropropan-2-yl)isoxazole-4-carboxylic acid (11.6 mg, 67.3 μmol, 1.00 eq) and was purified by prep-TLC (SiO₂, DCM:MeOH) to afford Compound 121, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(2-fluoropropan-2-yl)isoxazole-4-carboxamide, (17.93 mg, 29.71 μmol, 44% yield) as a yellow solid. LCMS [M+H]⁺=601.3 m/z.

### Example 125: Preparation of Compound 122

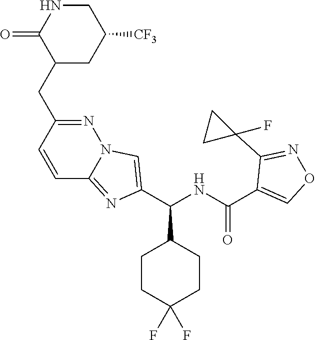 [SMILES: O=C(N[C@H](c1cn2nc(CC3C[C@@H](C(F)(F)F)CNC3=O)ccc2n1)C1CCC(F)(F)CC1)c1conc1C1(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(1-fluorocyclopropyl)isoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (156 mg, 350 μmol, 1.20 eq), 3-(1-fluorocyclopropyl)isoxazole-4-carboxylic acid (50.0 mg 292 μmol, 1.00 eq) and was purified by prep-HPLC (UniSil 3-100 C18 Ultra 150×25 mm, 3 um; mobile phase: [water (FA)-ACN]; B %: 47%-67%) to afford Compound 122, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(1-fluorocyclopropyl)isoxazole-4-carboxamide, (30.0 mg, 49.6 μmol, 17% yield) as a white solid. LCMS [M+H]⁺=599.2 m/z.

### Example 126: Preparation of Compound 123

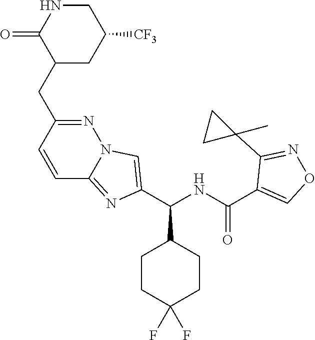 [SMILES: CC1(c2nocc2C(=O)N[C@H](c2cn3nc(CC4C[C@@H](C(F)(F)F)CNC4=O)ccc3n2)C2CCC(F)(F)CC2)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(1-methylcyclopropyl)isoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (35.0 mg, 78.5 μmol, 1.00 eq), 3-(1-methylcyclopropyl)isoxazole-4-carboxylic acid (14.4 mg, 86.4 μmol, 1.10 eq) and was purified by prep-HPLC (UniSil 3-100 C18 Ultra 150×25 mm, 3 um; mobile phase: [water (FA)-ACN]; B %: 49%-69%) to afford Compound 123, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(1-methylcyclopropyl)isoxazole-4-carboxamide (21.48 mg, 35.9 mol, 46% yield) as a brown solid. LCMS [M+H]⁺=595.2 m/z.

### Example 127: Preparation of Compound 124

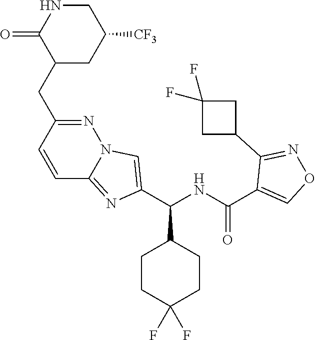 [SMILES: O=C(N[C@H](c1cn2nc(CC3C[C@@H](C(F)(F)F)CNC3=O)ccc2n1)C1CCC(F)(F)CC1)c1conc1C1CC(F)(F)C1]

3-(3,3-difluorocyclobutyl)-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)isoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (30.0 mg, 67.3 μmol, 1.00 eq), 3-(3,3-difluorocyclobutyl)isoxazole-4-carboxylic acid (12.3 mg, 60.6 μmol, 0.90 eq) and was purified by prep-HPLC (Phenomenexluna C18 150×25 mm, 10 μm; mobile phase: [water (FA)-ACN]; B %: 42%-72%) to afford Compound 124, 3-(3,3-difluorocyclobutyl)-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)isoxazole-4-carboxamide, (36.75 mg, 57.2 μmol, 85% yield) as a light green solid. LCMS [M+H]⁺=631.2 m z.

### Example 128: Preparation of Compound 125

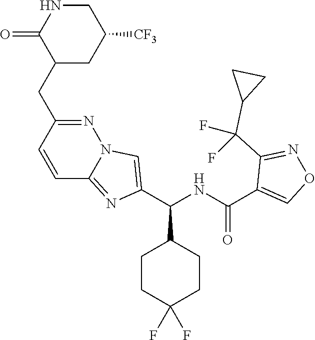 [SMILES: O=C(N[C@H](c1cn2nc(CC3C[C@@H](C(F)(F)F)CNC3=O)ccc2n1)C1CCC(F)(F)CC1)c1conc1C(F)(F)C1CC1]

3-(Cyclopropyldifluoromethyl)-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)isoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (30.0 mg, 67.3 μmol, 1.00 eq), 3-(cyclopropyldifluoromethyl)isoxazole-4-carboxylic acid (16.4 mg, 80.8 μmol, 1.20 eq) and was purified by prep-HPLC (Waters Xbridge 150 mm×25 mm, 5 um; mobile phase: [water (NH₄HCO₃)-ACN]; B %: 38%-68%) to afford Compound 125, 3-(cyclopropyldifluoromethyl)-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)isoxazole-4-carboxamide, (15.93 mg, 24.91 μmol, 37% yield) as a white solid. LCMS [M+H]⁺=631.3 m/z.

### Example 129: Preparation of Compounds 126 and 127

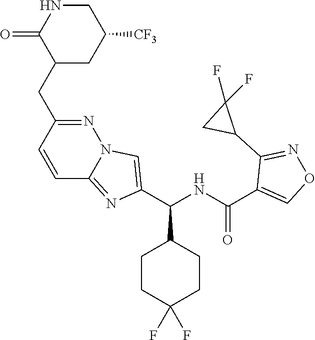 [SMILES: O=C(N[C@H](c1cn2nc(CC3C[C@@H](C(F)(F)F)CNC3=O)ccc2n1)C1CCC(F)(F)CC1)c1conc1C1CC1(F)F]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(2,2-difluorocyclopropyl)isoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (100 mg, 224 μmol, 1.00 eq), 3-(2,2-difluorocyclopropyl)isoxazole-4-carboxylic acid (63.6 mg, 336 μmol, 1.50 eq) and was purified by prep-TLC (SiO₂, DCM:MeOH) to afford N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(2,2-difluorocyclopropyl)isoxazole-4-carboxamide (85.0 mg, 137 mol, 61% yield) as a yellow solid. LCMS [M+H]⁺=617.4 m/z.

The title compound Compound 126 was isolated as the first eluting, single stereoisomer by chiral SFC purification (Daicel Chiralpak AS-H (250 mm×30 mm, 5 um); mobile phase: [CO₂—0.1% NH₃H₂O in MeOH]; B %: 25%); (27.80 mg, 45.09 μmol, 3 3% yield) and was obtained as a white solid. LCMS [M+H]⁺=617.3 m/z.

The title compound Compound 127 was isolated as the second eluting, single stereoisomer by chiral SFC purification (Daicel Chiralpak AS-H (250 mm×30 mm, 5 um); mobile phase: [CO₂—0.1% NH₃H₂O in MeOH]; B %: 25%); (44.04 mg, 71.43 μmol, 52% yield) and was obtained as a white solid. LCMS [M+H]⁺=617.2 m/z.

### Example 130: Preparation of Compound 128

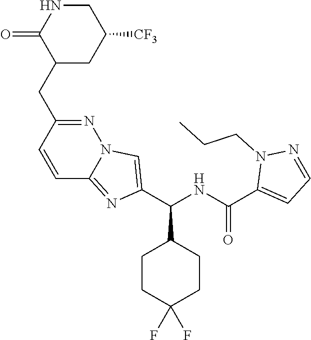 [SMILES: CCCn1nccc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-propyl-1H-pyrazole-5-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (20.0 mg, 44.9 μmol, 1.00 eq), 1-propyl-1H-pyrazole-5-carboxylic acid (7.61 mg, 49.3 μmol, 1.10 eq) and was purified by prep-HPLC (UniSil 3-100 C18 Ultra 150×25 mm, 3 um; mobile phase: [water (FA)-ACN]; B %: 48%-68%) to afford Compound 128, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-propyl-1H-pyrazole-5-carboxamide, (11.21 mg, 18.8 μmol, 42% yield) as a yellow solid. LCMS [M+H]⁺=582.2 m/z.

### Example 131: Preparation of Compound 129

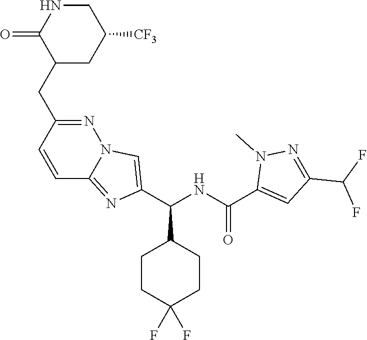 [SMILES: Cn1nc(C(F)F)cc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(difluoromethyl)-1-methyl-1H-pyrazole-5-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (30.0 mg, 67.4 μmol, 1.00 eq), 3-(difluoromethyl)-1-methyl-1H-pyrazole-5-carboxylic acid (17.8 mg, 101 μmol, 1.50 eq) and was purified by prep-TLC (SiO₂, DCM:MeOH) to afford Compound 129, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(difluoromethyl)-1-methyl-1H-pyrazole-5-carboxamide, (33.0 mg, 54.6 μmol, 81% yield) as a white solid. LCMS [M+H]⁺=604.3 m/z.

### Example 132: Preparation of Compounds 130 and 131

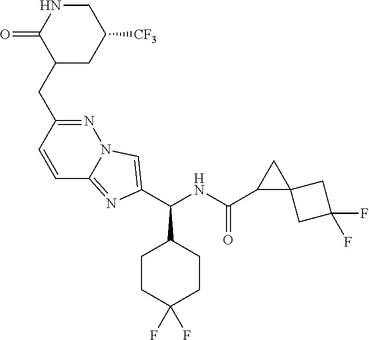 [SMILES: O=C1NC[C@H](C(F)(F)F)CC1Cc1ccc2nc([C@@H](NC(=O)C3CC34CC(F)(F)C4)C3CCC(F)(F)CC3)cn2n1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-5,5-difluorospiro[2.3]hexane-1-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (50.0 mg, 112 μmol, 1.00 eq), 5,5-difluorospiro[2.3]hexane-1-carboxylic acid (21.8 mg 134 μmol, 1.20 eq) and was purified by prep-HPLC (FA condition; Phenomenex luna C18 150×25 mm, 10 μm; mobile phase: [water (FA)-ACN]; B %: 35%-65%) to afford N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-5,5-difluorospiro[2.3]hexane-1-carboxamide (40.0 mg, 67.3 μmol, 60% yield) as a white solid. LCMS [M+H]⁺=590.3 m/z.

The title compound Compound 130 was isolated as the first eluting, single stereoisomer by chiral SFC purification (Daicel Chiralpak AD (250 mm×30 mm, 10 mm); mobile phase: [NH₃H₂O in EtOH]; B %: 35%-35%); (15.78 mg, 24.4 μmol, 36% yield) and was obtained as a white solid. LCMS [M+H]⁺=590.3 m/z.

The title compound Compound 131 was isolated as the second eluting, single stereoisomer by chiral SFC purification (Daicel Chiralpak AD (250 mm×30 mm, 10 mm); mobile phase: [NH₃H₂O in EtOH]; B %: 35%-35%); (23.02 mg, 37.1 μmol, 55% yield) and was obtained as a white solid. LCMS [M+H]⁺=590.3 m/z.

### Example 133: Preparation of Compound 132

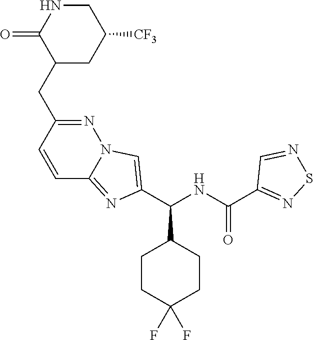 [SMILES: O=C(N[C@H](c1cn2nc(CC3C[C@@H](C(F)(F)F)CNC3=O)ccc2n1)C1CCC(F)(F)CC1)c1cnsn1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1,2,5-thiadiazole-3-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (15.0 mg, 33.7 μmol, 1.00 eq), 1,2,5-thiadiazole-3-carboxylic acid (10.1 mg, 77.5 μmol, 2.30 eq) and was purified by prep-TLC (SiO₂, DCM:MeOH) to afford Compound 132, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1,2,5-thiadiazole-3-carboxamide, (16.55 mg, 29.0 μmol, 86% yield) as a white solid. LCMS [M+H]⁺=558.3 m/z.

### Example 134: Preparation of Compound 133

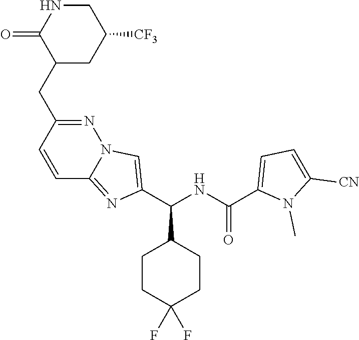 [SMILES: Cn1c(C#N)ccc1C(=O)N[C@H](c1cn2nc(CC3C[C@@H](C(F)(F)F)CNC3=O)ccc2n1)C1CCC(F)(F)CC1]

5-cyano-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-methyl-1H-pyrrole-2-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (15.0 mg, 33.7 μmol, 1.00 eq), 5-cyano-1-methyl-1H-pyrrole-2-carboxylic acid (7.58 mg 50.5 μmol, 1.50 eq) and was purified by prep-HPLC (Waters Xbridge 150 mm×25 mm, 5 um; mobile phase: [water (NH₄HCO₃)-ACN]; B %: 36%-66%) to afford Compound 133, 5-cyano-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-methyl-1H-pyrrole-2-carboxamide, (8.96 mg 14.1 μmol, 42% yield) as a white solid. LCMS [M+H]⁺=578.2 m/z.

### Example 135: Preparation of Compound 134

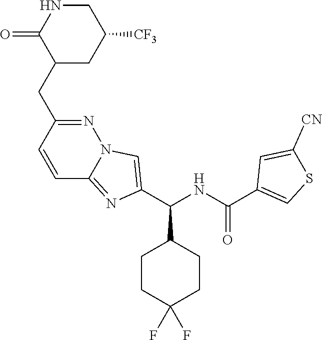 [SMILES: N#Cc1cc(C(=O)N[C@H](c2cn3nc(CC4C[C@@H](C(F)(F)F)CNC4=O)ccc3n2)C2CCC(F)(F)CC2)cs1]

5-cyano-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)thiophene-3-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (30.0 mg, 67.3 μmol, 1.00 eq), 5-cyanothiophene-3-carboxylic acid (11.3 mg, 74.0 μmol, 1.10 eq) and was purified by prep-HPLC (FA condition; Phenomenexluna C18 150×25 mm, 10 μm; mobile phase: [water (FA)-ACN]; B %: 35%-65%) to afford Compound 134, 5-cyano-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)thiophene-3-carboxamide, (7.36 mg, 12.2 μmol, 18% yield) as a white solid. LCMS [M+H]⁺=581.2 m/z.

### Example 136: Preparation of Compound 135

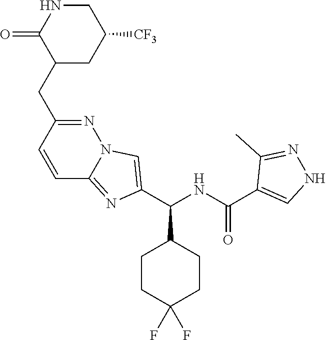 [SMILES: Cc1n[nH]cc1C(=O)N[C@H](c1cn2nc(CC3C[C@@H](C(F)(F)F)CNC3=O)ccc2n1)C1CCC(F)(F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-methyl-1H-pyrazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (20.0 mg, 44.9 μmol, 1.00 eq), 3-methyl-1H-pyrazole-4-carboxylic acid (5.66 mg, 44.9 μmol, 1.00 eq) and was purified by prep-TLC (SiO₂, DCM:MeOH) to afford Compound 135, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-methyl-1H-pyrazole-4-carboxamide (9.96 mg, 17.5 μmol, 39% yield) as a yellow solid. LCMS [M+H]⁺=554.3 m/z.

### Example 137: Preparation of Compound 136

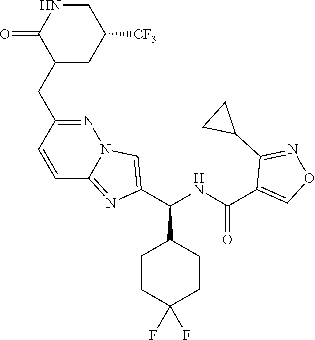 [SMILES: O=C(N[C@H](c1cn2nc(CC3C[C@@H](C(F)(F)F)CNC3=O)ccc2n1)C1CCC(F)(F)CC1)c1conc1C1CC1]

3-Cyclopropyl-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)isoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (50.0 mg, 112 μmol, 1.00 eq), 3-cyclopropylisoxazole-4-carboxylic acid (25.8 mg, 168 μmol, 1.50 eq) and was purified by prep-TLC (SiO₂, DCM:MeOH) to afford Compound 136, 3-cyclopropyl-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)isoxazole-4-carboxamide, (18.91 mg, 32.43 mol, 29% yield) as a white solid. LCMS [M+H]⁺=581.4 m/z.

### Example 138: Preparation of Compound 137

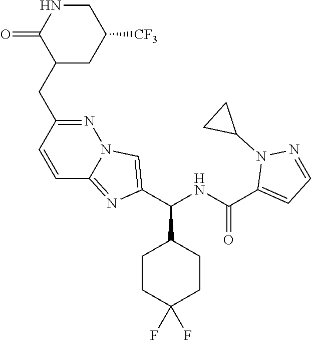 [SMILES: O=C(N[C@H](c1cn2nc(CC3C[C@@H](C(F)(F)F)CNC3=O)ccc2n1)C1CCC(F)(F)CC1)c1ccnn1C1CC1]

1-Cyclopropyl-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1H-pyrazole-5-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (40.0 mg, 89.8 μmol, 1.00 eq), 1-cyclopropyl-1H-pyrazole-5-carboxylic acid (20.4 mg 134 μmol, 1.50 eq) and was purified by prep-TLC (SiO₂, DCM:MeOH) to afford Compound 137, 1-cyclopropyl-N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1H-pyrazole-5-carboxamide, (37.08 mg, 63.53 mol, 71% yield) as a white solid. LCMS [M+H]⁺=580.4 m/z.

### Example 139: Preparation of Compound 138

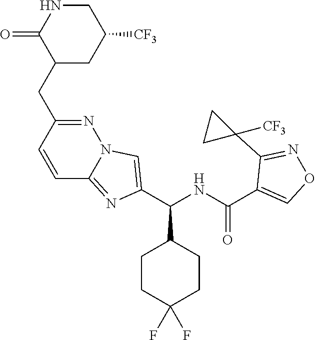 [SMILES: O=C(N[C@H](c1cn2nc(CC3C[C@@H](C(F)(F)F)CNC3=O)ccc2n1)C1CCC(F)(F)CC1)c1conc1C1(C(F)(F)F)CC1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(1-(trifluoromethyl)cyclopropyl)isoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (40.0 mg, 89.8 μmol, 1.00 eq), 3-(1-(trifluoromethyl)cyclopropyl)isoxazole-4-carboxylic acid (51.6 mg, 269 μmol, 3.00 eq) and was purified by prep-TLC (SiO₂, DCM:MeOH) to afford Compound 138, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(1-(trifluoromethyl)cyclopropyl)isoxazole-4-carboxamide, (0.045 g, 68.9 μmol, 77% yield) as a yellow solid. LCMS [M+H]⁺=649.3 m/z.

### Example 140: Preparation of Compound 139

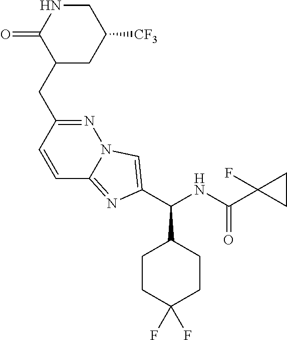 [SMILES: O=C1NC[C@H](C(F)(F)F)CC1Cc1ccc2nc([C@@H](NC(=O)C3(F)CC3)C3CCC(F)(F)CC3)cn2n1]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-fluorocyclopropane-1-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (30.0 mg, 67.3 μmol, 1.00 eq), 1-fluorocyclopropane-1-carboxylic acid (8.41 mg, 80.8 mol, 1.20 eq) and was purified by prep-TLC (SiO₂, DCM:MeOH) to afford Compound 139, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-fluorocyclopropane-1-carboxamide, (23.5 mg, 43.4 μmol, 65% yield) as a brown solid. LCMS [M+H]⁺=532.4 m/z.

### Example 141: Preparation of Compound 140

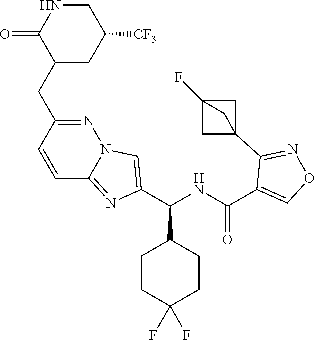 [SMILES: O=C(N[C@H](c1cn2nc(CC3C[C@@H](C(F)(F)F)CNC3=O)ccc2n1)C1CCC(F)(F)CC1)c1conc1C12CC(F)(C1)C2]

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(3-fluorobicyclo[1.1.1]pentan-1-yl)isoxazole-4-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (20.0 mg, 44.9 μmol, 1.00 eq), 3-(3-fluorobicyclo[1.1.1]pentan-1-yl)isoxazole-4-carboxylic acid (10.6 mg, 53.9 μmol, 1.20 eq) and was purified by prep-TLC (SiO₂, DCM:MeOH) to afford Compound 140, N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-3-(3-fluorobicyclo[1.1.1]pentan-1-yl)isoxazole-4-carboxamide, (21.76 mg, 33.5 μmol, 75% yield) as a white solid. LCMS [M+H]⁺=625.3 m/z.

### Example 142: Preparation of Compound 141

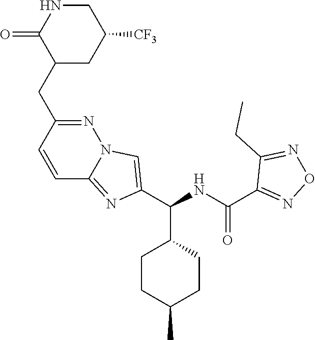 [SMILES: CCc1nonc1C(=O)N[C@H](c1cn2nc(CC3C[C@@H](C(F)(F)F)CNC3=O)ccc2n1)[C@H]1CC[C@H](C)CC1]

4-Ethyl-N-((1S)-((1R.4S)-4-methylcyclohexyl)(6-(((5R)-2-oxo-5-(trifluoromethyl)piperidin-3-yl)methyl)imidazo[1,2-b]pyridazin-2-yl)methyl)-1,2,5-oxadiazole-3-carboxamide was synthesized following the General Procedure 4 with (5R)-3-((2-((S)-amino((1R.4S)-4-methylcyclohexyl)methyl)imidazo[1,2-b]pyridazin-6-yl)methyl)-5-(trifluoromethyl)piperidin-2-one (120 mg, 283 μmol, 1.00 eq), 4-ethyl-1,2,5-oxadiazole-3-carboxylic acid (48.3 mg, 340 μmol, 1.20 eq) and was purified by prep-HPLC (UniSil3—100 C18 Ultra 150×25 mm, 3 um; mobile phase: [water (FA)-ACN]; B %: 61%-81%) to afford Compound 141, 4-ethyl-N-((1S)—((1R.4S)-4-methylcyclohexyl)(6-(((5R)-2-oxo-5-(trifluoromethyl)piperidin-3-yl)methyl)imidazo[1,2-b]pyridazin-2-yl)methyl)-1,2,5-oxadiazole-3-carboxamide, (30.0 mg, 54.3 μmol, 19% yield) as a white solid. LCMS [M+H]⁺=548.4 m/z.

### Example 143: Preparation of Compound 142

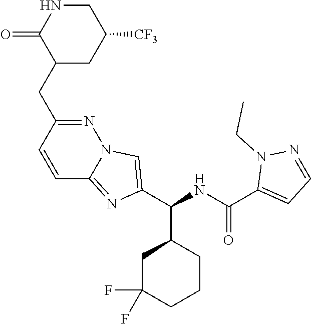 [SMILES: CCn1nccc1C(=O)N[C@H](c1cn2nc(CC3C[C@@H](C(F)(F)F)CNC3=O)ccc2n1)[C@H]1CCCC(F)(F)C1]

N-((1S)—((S)-3,3-difluorocyclohexyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5R)-3-((2-((S)-amino((S)-3,3-difluorocyclohexyl)methyl)imidazo[1,2-b]pyridazin-6-yl)methyl)-5-(trifluoromethyl)piperidin-2-one (60.0 mg, 135 μmol, 1.00 eq), 1-ethyl-1H-pyrazole-5-carboxylic acid (37.8 mg, 269 μmol, 2.00 eq) and was purified by prep-TLC (SiO₂, DCM:MeOH) to afford Compound 142, N-((1S)—((S)-3,3-difluorocyclohexyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, (43.8 mg 77.1 μmol, 57% yield) as a white solid. LCMS [M+H]⁺=568.4 m/z.

### Example 144: Preparation of Compound 143

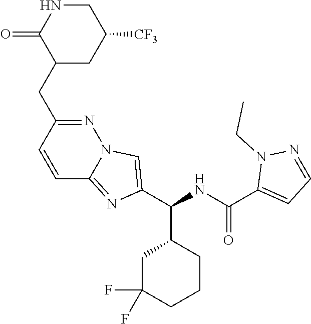 [SMILES: CCn1nccc1C(=O)N[C@H](c1cn2nc(CC3C[C@@H](C(F)(F)F)CNC3=O)ccc2n1)[C@@H]1CCCC(F)(F)C1]

N-((1S)—((R)-3,3-difluorocyclohexyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5R)-3-((2-((S)-amino((R)-3,3-difluorocyclohexyl)methyl)imidazo[1,2-b]pyridazin-6-yl)methyl)-5-(trifluoromethyl)piperidin-2-one (30.0 mg, 67.3 μmol, 1.00 eq), 1-ethyl-1H-pyrazole-5-carboxylic acid (18.9 mg, 135 μmol, 2.00 eq) and was purified by prep-TLC (SiO₂, DCM:MeOH) to afford Compound 143, N-((1S)—((R)-3,3-difluorocyclohexyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, (15.0 mg 26.0 μmol, 49% yield,) as a white solid. LCMS [M+H]⁺=568.3 m/z.

### Example 145: Preparation of Compound 144

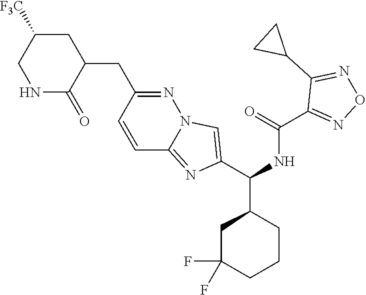 [SMILES: O=C(N[C@H](c1cn2nc(CC3C[C@@H](C(F)(F)F)CNC3=O)ccc2n1)[C@H]1CCCC(F)(F)C1)c1nonc1C1CC1]

4-Cyclopropyl-N—((S)-((S)-3,3-difluorocyclohexyl)(6-(((5R)-2-oxo-5-(trifluoromethyl)piperidin-3-yl)methyl)imidazo[1,2-b]pyridazin-2-yl)methyl)-1,2,5-oxadiazole-3-carboxamide was synthesized following the General Procedure 4 with (5R)-3-((2-((S)-amino((S)-3,3-difluorocyclohexyl)methyl)imidazo[1,2-b]pyridazin-6-yl)methyl)-5-(trifluoromethyl)piperidin-2-one (30.0 mg, 67.4 μmol, 1.00 eq), 4-cyclopropyl-1,2,5-oxadiazole-3-carboxylic acid (15.6 mg, 101 μmol, 1.50 eq) and was purified by prep-TLC (SiO₂, DCM:MeOH) to afford Compound 144, 4-cyclopropyl-N—((S)-((S)-3,3-difluorocyclohexyl)(6-(((5R)-2-oxo-5-(trifluoromethyl)piperidin-3-yl)methyl)imidazo[1,2-b]pyridazin-2-yl)methyl)-1,2,5-oxadiazole-3-carboxamide, (20 mg, 34.0 μmol, 51% yield) as a white solid. LCMS [M+H]+=582.3 m/z.

### Example 146: Preparation of Compound 145

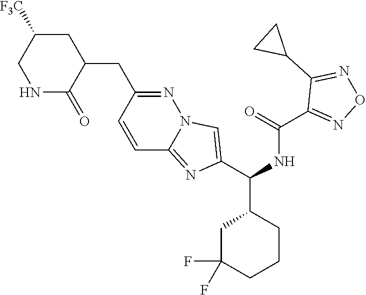 [SMILES: O=C(N[C@H](c1cn2nc(CC3C[C@@H](C(F)(F)F)CNC3=O)ccc2n1)[C@@H]1CCCC(F)(F)C1)c1nonc1C1CC1]

4-Cyclopropyl-N—((S)-((R)-3,3-difluorocyclohexyl)(6-(((5R)-2-oxo-5-(trifluoromethyl)piperidin-3-yl)methyl)imidazo[1,2-b]pyridazin-2-yl)methyl)-1,2,5-oxadiazole-3-carboxamide was synthesized following the General Procedure 4 with (5R)-3-((2-((S)-amino((R)-3,3-difluorocyclohexyl)methyl)imidazo[1,2-b]pyridazin-6-yl)methyl)-5-(trifluoromethyl)piperidin-2-one (20.0 mg, 44.9 μmol, 1.00 eq), 4-cyclopropyl-1,2,5-oxadiazole-3-carboxylic acid (10.4 mg, 67.3 μmol, 1.50 eq) and was purified by prep-TLC (SiO₂, DCM:MeOH) to afford Compound 145, 4-cyclopropyl-N—((S)-((R)-3,3-difluorocyclohexyl)(6-(((5R)-2-oxo-5-(trifluoromethyl)piperidin-3-yl)methyl)imidazo[1,2-b]pyridazin-2-yl)methyl)-1,2,5-oxadiazole-3-carboxamide, (9.37 mg, 15.6 μmol, 35% yield) as a white solid. LCMS [M+H]⁺=582.4 m/z.

### Example 147: Preparation of Compound 146

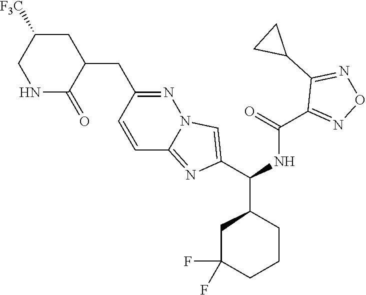 [SMILES: O=C(N[C@H](c1cn2nc(CC3C[C@@H](C(F)(F)F)CNC3=O)ccc2n1)[C@H]1CCCC(F)(F)C1)c1nonc1C1CC1]

3-Cyclopropyl-N-((1S)—((S)-3,3-difluorocyclohexyl)(6-(((5R)-2-oxo-5-(trifluoromethyl)piperidin-3-yl)methyl)imidazo[1,2-b]pyridazin-2-yl)methyl)isoxazole-4-carboxamide was synthesized following the General Procedure 4 with (5R)-3-((2-((S)-amino((S)-3,3-difluorocyclohexyl)methyl)imidazo[1,2-b]pyridazin-6-yl)methyl)-5-(trifluoromethyl)piperidin-2-one (25.0 mg, 56.1 μmol, 1.00 eq), 3-cyclopropylisoxazole-4-carboxylic acid (12.9 mg, 84.2 μmol, 1.50 eq) and was purified by prep-TLC (SiO₂, DCM:MeOH) to afford Compound 146, 3-cyclopropyl-N-((1S)—((S)-3,3-difluorocyclohexyl)(6-(((5R)-2-oxo-5-(trifluoromethyl)piperidin-3-yl)methyl)imidazo[1,2-b]pyridazin-2-yl)methyl)isoxazole-4-carboxamide, (20 mg, 34.4 μmol, 61% yield) as a white solid. LCMS [M+H]⁺=581.2 m/z.

### Example 148: Preparation of Compound 147

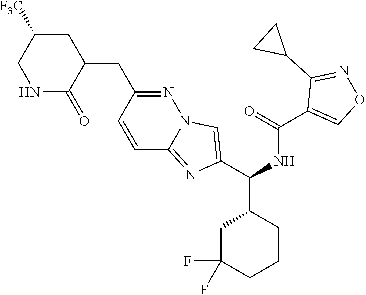 [SMILES: O=C(N[C@H](c1cn2nc(CC3C[C@@H](C(F)(F)F)CNC3=O)ccc2n1)[C@@H]1CCCC(F)(F)C1)c1conc1C1CC1]

3-Cyclopropyl-N-((1S)—((R)-3,3-difluorocyclohexyl)(6-(((5R)-2-oxo-5-(trifluoromethyl)piperidin-3-yl)methyl)imidazo[1,2-b]pyridazin-2-yl)methyl)isoxazole-4-carboxamide was synthesized following the General Procedure 4 with (5R)-3-((2-((S)-amino((R)-3,3-difluorocyclohexyl)methyl)imidazo[1,2-b]pyridazin-6-yl)methyl)-5-(trifluoromethyl)piperidin-2-one (20.0 mg, 44.9 μmol, 1.00 eq), 3-cyclopropylisoxazole-4-carboxylic acid (10.3 mg, 67.4 μmol, 1.50 eq) and was purified by prep-TLC (SiO₂, DCM:MeOH) to afford Compound 147, 3-cyclopropyl-N-((1S)—((R)-3,3-difluorocyclohexyl)(6-(((5R)-2-oxo-5-(trifluoromethyl)piperidin-3-yl)methyl)imidazo[1,2-b]pyridazin-2-yl)methyl)isoxazole-4-carboxamide, (14.2 mg, 23.7 μmol, 53% yield) as a white solid. LCMS [M+H]⁺=581.4 m/z.

### Example 149: Preparation of Compound 148

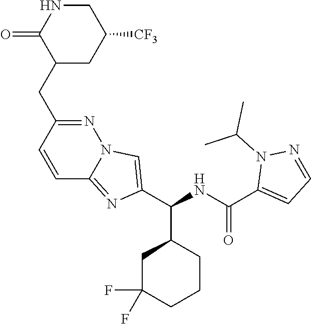 [SMILES: CC(C)n1nccc1C(=O)N[C@H](c1cn2nc(CC3C[C@@H](C(F)(F)F)CNC3=O)ccc2n1)[C@H]1CCCC(F)(F)C1]

N-((1S)—((S)-3,3-difluorocyclohexyl)(6-(((5R)-2-oxo-5-(trifluoromethyl)piperidin-3-yl)methyl)imidazo[1,2-b]pyridazin-2-yl)methyl)-1-isopropyl-1H-pyrazole-5-carboxamide was synthesized following the General Procedure 4 with (5R)-3-((2-((S)-amino((S)-3,3-difluorocyclohexyl)methyl)imidazo[1,2-b]pyridazin-6-yl)methyl)-5-(trifluoromethyl)piperidin-2-one (20.0 mg, 44.9 μmol, 1.00 eq), 1-isopropyl-1H-pyrazole-5-carboxylic acid (8.31 mg, 53.8 mol, 1.20 eq) and was purified by prep-HPLC (Phenomenexluna C18 150×25 mm, 10 μm; mobile phase: [water (FA)-ACN]; B %: 37%-67%) to afford Compound 148, N-((1S)—((S)-3,3-difluorocyclohexyl)(6-(((5R)-2-oxo-5-(trifluoromethyl)piperidin-3-yl)methyl)imidazo[1,2-b]pyridazin-2-yl)methyl)-1-isopropyl-1H-pyrazole-5-carboxamide, (5.20 mg, 8.58 μmol, 19% yield) as a white solid. LCMS [M+H]⁺=582.3 m/z.

### Example 150: Preparation of Compound 149

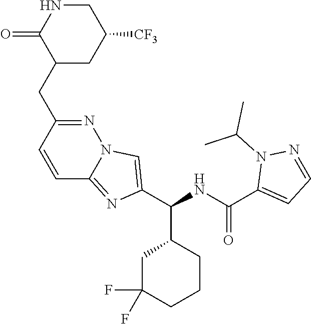 [SMILES: CC(C)n1nccc1C(=O)N[C@H](c1cn2nc(CC3C[C@@H](C(F)(F)F)CNC3=O)ccc2n1)[C@@H]1CCCC(F)(F)C1]

N-((1S)—((R)-3,3-difluorocyclohexyl)(6-(((5R)-2-oxo-5-(trifluoromethyl)piperidin-3-yl)methyl)imidazo[1,2-b]pyridazin-2-yl)methyl)-1-isopropyl-1H-pyrazole-5-carboxamide was synthesized following the General Procedure 4 with (5R)-3-((2-((S)-amino((R)-3,3-difluorocyclohexyl)methyl)imidazo[1,2-b]pyridazin-6-yl)methyl)-5-(trifluoromethyl)piperidin-2-one (20.0 mg, 44.9 μmol, 1.00 eq), 1-isopropyl-1H-pyrazole-5-carboxylic acid (10.4 mg, 67.4 mol, 1.50 eq) and was purified by prep-TLC (SiO₂, DCM:MeOH) to afford Compound 149, N-((1S)—((R)-3,3-difluorocyclohexyl)(6-(((5R)-2-oxo-5-(trifluoromethyl)piperidin-3-yl)methyl)imidazo[1,2-b]pyridazin-2-yl)methyl)-1-isopropyl-1H-pyrazole-5-carboxamide, (14.9 mg, 25.7 μmol, 57% yield) as a white solid. LCMS [M+H]⁺=582.3 m/z.

### Example 151: Preparation of Compound 150

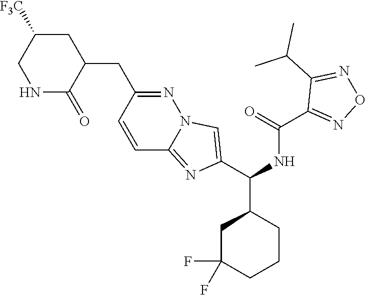 [SMILES: CC(C)c1nonc1C(=O)N[C@H](c1cn2nc(CC3C[C@@H](C(F)(F)F)CNC3=O)ccc2n1)[C@H]1CCCC(F)(F)C1]

N-((1S)—((S)-3,3-difluorocyclohexyl)(6-(((5R)-2-oxo-5-(trifluoromethyl)piperidin-3-yl)methyl)imidazo[1,2-b]pyridazin-2-yl)methyl)-4-isopropyl-1,2,5-oxadiazole-3-carboxamide was synthesized following the General Procedure 4 with (5R)-3-((2-((S)-amino((S)-3,3-difluorocyclohexyl)methyl)imidazo[1,2-b]pyridazin-6-yl)methyl)-5-(trifluoromethyl)piperidin-2-one (50.0 mg, 112 μmol, 1.00 eq), 4-isopropyl-1,2,5-oxadiazole-3-carboxylic acid (26.3 mg 168 μmol, 1.50 eq) and was purified by prep-TLC (SiO₂, DCM:MeOH) to afford Compound 150, N-((1S)—((S)-3,3-difluorocyclohexyl)(6-(((5R)-2-oxo-5-(trifluoromethyl)piperidin-3-yl)methyl)imidazo[1,2-b]pyridazin-2-yl)methyl)-4-isopropyl-1,2,5-oxadiazole-3-carboxamide, (23 mg, 38.9 μmol, 35% yield) as an orange solid. LCMS [M+H]⁺=584.3 m/z.

### Example 152: Preparation of Compound 151

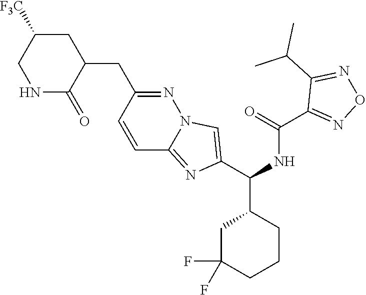 [SMILES: CC(C)c1nonc1C(=O)N[C@H](c1cn2nc(CC3C[C@@H](C(F)(F)F)CNC3=O)ccc2n1)[C@@H]1CCCC(F)(F)C1]

N-((1S)—((R)-3,3-difluorocyclohexyl)(6-(((5R)-2-oxo-5-(trifluoromethyl)piperidin-3-yl)methyl)imidazo[1,2-b]pyridazin-2-yl)methyl)-4-isopropyl-1,2,5-oxadiazole-3-carboxamide was synthesized following the General Procedure 4 with 5R)-3-((2-((S)-amino((R)-3,3-difluorocyclohexyl)methyl)imidazo[1,2-b]pyridazin-6-yl)methyl)-5-(trifluoromethyl)piperidin-2-one (25.0 mg, 56.1 μmol, 1.00 eq), 4-isopropyl-1,2,5-oxadiazole-3-carboxylic acid (13.1 mg, 84.1 μmol, 1.50 eq) and was purified by prep-TLC (SiO₂, DCM:MeOH) to afford Compound 151, N-((1S)—((R)-3,3-difluorocyclohexyl)(6-(((5R)-2-oxo-5-(trifluoromethyl)piperidin-3-yl)methyl)imidazo[1,2-b]pyridazin-2-yl)methyl)-4-isopropyl-1,2,5-oxadiazole-3-carboxamide, (14.6 mg, 24.4 μmol, 43% yield) as a white solid. LCMS [M+H]⁺=584.4 m/z.

### Example 153: Preparation of Compound 152

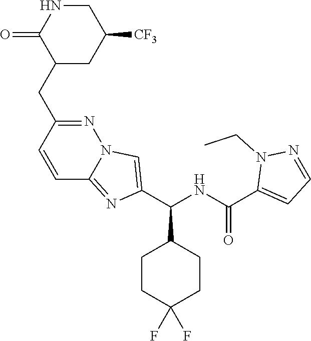 [SMILES: CCn1nccc1C(=O)N[C@H](c1cn2nc(CC3C[C@H](C(F)(F)F)CNC3=O)ccc2n1)C1CCC(F)(F)CC1]

N-((1S)-(4,4-difluorocyclohexyl)(6-(((5S)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5S)-3-((2-((S)-amino(4,4-difluorocyclohexyl)methyl)imidazo[1,2-b]pyridazin-6-yl)methyl)-5-(trifluoromethyl)piperidin-2-one (70.0 mg, 157 μmol, 1.00 eq), 1-ethyl-1H-pyrazole-5-carboxylic acid (26.4 mg, 188 μmol, 1.20 eq) and was purified by prep-TLC (SiO₂, DCM:MeOH) to afford Compound 152, N-((1S)-(4,4-difluorocyclohexyl)(6-(((5S)-2-oxo-5-(trifluoromethyl)piperidin-3-yl)methyl)imidazo[1,2-b]pyridazin-2-yl)methyl)-1-ethyl-1H-pyrazole-5-carboxamide, (44.8 mg, 76.4 μmol, 49% yield) as a white solid. LCMS [M+H]⁺=568.3 m/z.

### Example 154: Preparation of Compound 153

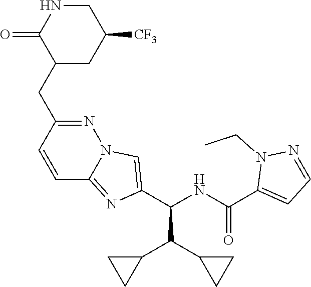 [SMILES: CCn1nccc1C(=O)N[C@H](c1cn2nc(CC3C[C@H](C(F)(F)F)CNC3=O)ccc2n1)C(C1CC1)C1CC1]

N-((1S)-2,2-dicyclopropyl-1-(6-(((5S)-2-oxo-5-(trifluoromethyl)piperidin-3-yl)methyl)imidazo[1,2-b]pyridazin-2-yl)ethyl)-1-ethyl-1H-pyrazole-5-carboxamide was synthesized following the General Procedure 4 with (5S)-3-((2-((S)-1-amino-2,2-dicyclopropylethyl)imidazo[1,2-b]pyridazin-6-yl)methyl)-5-(trifluoromethyl)piperidin-2-one (50.0 mg, 118 μmol, 1.00 eq), 1-ethyl-1H-pyrazole-5-carboxylic acid (19.9 mg, 142 μmol, 1.20 eq) and was purified by prep-HPLC (Phenomenex luna C18 150×25 mm, 10 μm; mobile phase: [water (FA)-ACN]; B %: 38%-68%) to afford Compound 153, N-((1S)-2,2-dicyclopropyl-1-(6-(((5S)-2-oxo-5-(trifluoromethyl)piperidin-3-yl)methyl)imidazo[1,2-b]pyridazin-2-yl)ethyl)-1-ethyl-1H-pyrazole-5-carboxamide, (35.37 mg, 62.6 μmol, 53% yield) as a yellow solid. LCMS [M+H]⁺=544.3 m/z.

### Example 155: Preparation of Compound 154

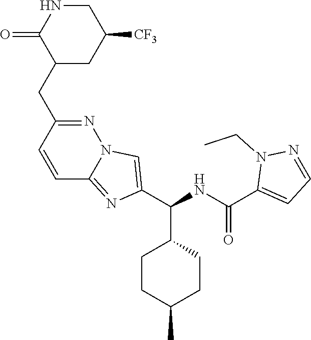 [SMILES: CCn1nccc1C(=O)N[C@H](c1cn2nc(CC3C[C@H](C(F)(F)F)CNC3=O)ccc2n1)[C@H]1CC[C@H](C)CC1]

1-Ethyl-N-((1S)-((1R.4S)-4-methylcyclohexyl)(6-(((5S)-2-oxo-5-(trifluoromethyl)piperidin-3-yl)methyl)imidazo[1,2-b]pyridazin-2-yl)methyl)-1H-pyrazole-5-carboxamide was synthesized following the General Procedure 4 with (5S)-3-((2-((S)-amino((1R.4S)-4-methylcyclohexyl)methyl)imidazo[1,2-b]pyridazin-6-yl)methyl)-5-(trifluoromethyl)piperidin-2-one (50.0 mg, 118 μmol, 1.00 eq), 1-ethyl-1H-pyrazole-5-carboxylic acid (24.8 mg, 177 μmol, 1.50 eq) and was purified by prep-TLC (SiO₂, DCM:MeOH) to afford Compound 154, 1-ethyl-N-((1S)—((1R.4S)-4-methylcyclohexyl)(6-(((5S)-2-oxo-5-(trifluoromethyl)piperidin-3-yl)methyl)imidazo[1,2-b]pyridazin-2-yl)methyl)-1H-pyrazole-5-carboxamide, (25.9 mg, 45.5 μmol, 39% yield) as a white solid. LCMS [M+H]⁺=546.3 m/z.

### Example 156: Preparation of Compound 155

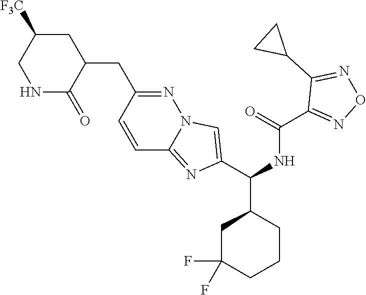 [SMILES: O=C(N[C@H](c1cn2nc(CC3C[C@H](C(F)(F)F)CNC3=O)ccc2n1)[C@H]1CCCC(F)(F)C1)c1nonc1C1CC1]

4-Cyclopropyl-N—((S)-((S)-3,3-difluorocyclohexyl)(6-(((5S)-2-oxo-5-(trifluoromethyl)piperidin-3-yl)methyl)imidazo[1,2-b]pyridazin-2-yl)methyl)-1,2,5-oxadiazole-3-carboxamide was synthesized following the General Procedure 4 with (5S)-3-((2-((S)-amino((S)-3,3-difluorocyclohexyl)methyl)imidazo[1,2-b]pyridazin-6-yl)methyl)-5-(trifluoromethyl)piperidin-2-one (30.0 mg, 67.3 μmol, 1.00 eq), 4-cyclopropyl-1,2,5-oxadiazole-3-carboxylic acid (12.4 mg, 80.8 μmol, 1.20 eq) and was purified by prep-TLC (SiO₂, DCM:MeOH) to afford Compound 155, 4-cyclopropyl-N—((S)-((S)-3,3-difluorocyclohexyl)(6-(((5S)-2-oxo-5-(trifluoromethyl)piperidin-3-yl)methyl)imidazo[1,2-b]pyridazin-2-yl)methyl)-1,2,5-oxadiazole-3-carboxamide, (15.8 mg, 26.8 μmol, 40% yield) as a white solid. LCMS [M+H]⁺=582.3 m/z.

### Example 157: Preparation of Compound 156

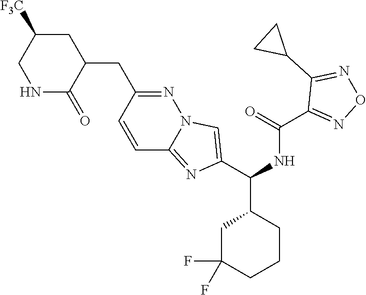 [SMILES: O=C(N[C@H](c1cn2nc(CC3C[C@H](C(F)(F)F)CNC3=O)ccc2n1)[C@@H]1CCCC(F)(F)C1)c1nonc1C1CC1]

4-Cyclopropyl-N—((S)-((R)-3,3-difluorocyclohexyl)(6-(((5S)-2-oxo-5-(trifluoromethyl)piperidin-3-yl)methyl)imidazo[1,2-b]pyridazin-2-yl)methyl)-1,2,5-oxadiazole-3-carboxamide was synthesized following the General Procedure 4 with (5S)-3-((2-((S)-amino((R)-3,3-difluorocyclohexyl)methyl)imidazo[1,2-b]pyridazin-6-yl)methyl)-5-(trifluoromethyl)piperidin-2-one (20.0 mg, 44.9 μmol, 1.00 eq), 4-cyclopropyl-1,2,5-oxadiazole-3-carboxylic acid (10.3 mg, 67.3 μmol, 1.50 eq) and was purified by prep-HPLC (Phenomenex luna C18 150×25 mm, 10 μm; mobile phase: [water (FA)-ACN]; B %: 25%-50%) to afford Compound 156, 4-cyclopropyl-N—((S)-((R)-3,3-difluorocyclohexyl)(6-(((5S)-2-oxo-5-(trifluoromethyl)piperidin-3-yl)methyl)imidazo[1,2-b]pyridazin-2-yl)methyl)-1,2,5-oxadiazole-3-carboxamide, (22.98 mg, 37.3 μmol, 83% yield) as a yellow solid. LCMS [M+H]⁺=582.3 m/z.

### Example 158: Preparation of Compound 157

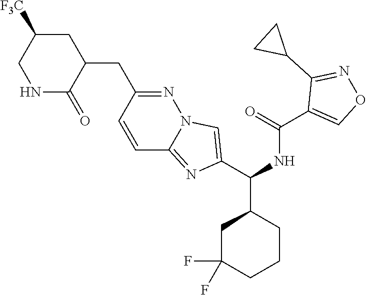 [SMILES: O=C(N[C@H](c1cn2nc(CC3C[C@H](C(F)(F)F)CNC3=O)ccc2n1)[C@H]1CCCC(F)(F)C1)c1conc1C1CC1]

3-Cyclopropyl-N-((1S)—((S)-3,3-difluorocyclohexyl)(6-(((5S)-2-oxo-5-(trifluoromethyl)piperidin-3-yl)methyl)imidazo[1,2-b]pyridazin-2-yl)methyl)isoxazole-4-carboxamide was synthesized following the General Procedure 4 with (5S)-3-((2-((S)-amino((S)-3,3-difluorocyclohexyl)methyl)imidazo[1,2-b]pyridazin-6-yl)methyl)-5-(trifluoromethyl)piperidin-2-one (30.0 mg, 67.3 μmol, 1.00 eq), 3-cyclopropylisoxazole-4-carboxylic acid (12.3 mg, 80.8 μmol, 1.20 eq) and was purified by prep-TLC (SiO₂, DCM:MeOH) to afford Compound 157, 3-cyclopropyl-N-((1S)—((S)-3,3-difluorocyclohexyl)(6-(((5S)-2-oxo-5-(trifluoromethyl)piperidin-3-yl)methyl)imidazo[1,2-b]pyridazin-2-yl)methyl)isoxazole-4-carboxamide, (12.1 mg, 20.6 μmol, 3 1% yield) as a white solid. LCMS [M+H]⁺=581.3 m/z.

### Example 159: Preparation of Compound 158

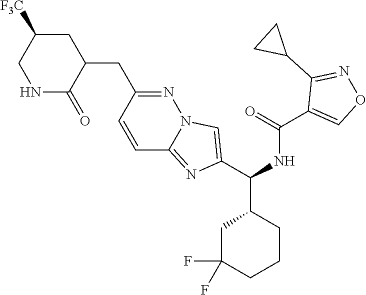 [SMILES: O=C(N[C@H](c1cn2nc(CC3C[C@H](C(F)(F)F)CNC3=O)ccc2n1)[C@@H]1CCCC(F)(F)C1)c1conc1C1CC1]

3-Cyclopropyl-N-((1S)—((R)-3,3-difluorocyclohexyl)(6-(((5S)-2-oxo-5-(trifluoromethyl)piperidin-3-yl)methyl)imidazo[1,2-b]pyridazin-2-yl)methyl)isoxazole-4-carboxamide was synthesized following the General Procedure 4 with (5S)-3-((2-((S)-amino((R)-3,3-difluorocyclohexyl)methyl)imidazo[1,2-b]pyridazin-6-yl)methyl)-5-(trifluoromethyl)piperidin-2-one (20.0 mg, 44.9 μmol, 1.00 eq), 3-cyclopropylisoxazole-4-carboxylic acid (10.3 mg, 67.3 μmol, 1.50 eq) and was purified by prep-HPLC (Phenomenex luna C18 150×40 mm, 15 um; mobile phase: [water (FA)-ACN]; B %: 41%-71%) to afford Compound 158, 3-cyclopropyl-N-((1S)—((R)-3,3-difluorocyclohexyl)(6-(((5S)-2-oxo-5-(trifluoromethyl)piperidin-3-yl)methyl)imidazo[1,2-b]pyridazin-2-yl)methyl)isoxazole-4-carboxamide, (14.35 mg, 24.6 μmol, 95% yield) as a white solid. LCMS [M+H]⁺=581.4 m/z.

### Example 160: Preparation of Compound 159

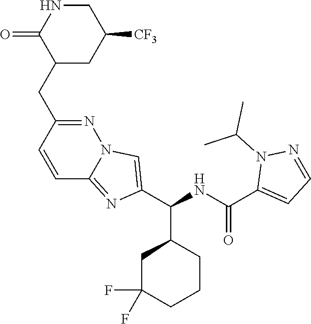 [SMILES: CC(C)n1nccc1C(=O)N[C@H](c1cn2nc(CC3C[C@H](C(F)(F)F)CNC3=O)ccc2n1)[C@H]1CCCC(F)(F)C1]

N-((1S)—((S)-3,3-difluorocyclohexyl)(6-(((5S)-2-oxo-5-(trifluoromethyl)piperidin-3-yl)methyl)imidazo[1,2-b]pyridazin-2-yl)methyl)-1-isopropyl-1H-pyrazole-5-carboxamide was synthesized following the General Procedure 4 with (5S)-3-((2-((S)-amino((S)-3,3-difluorocyclohexyl)methyl)imidazo[1,2-b]pyridazin-6-yl)methyl)-5-(trifluoromethyl)piperidin-2-one (30.0 mg, 67.3 μmol, 1.00 eq), 1-isopropyl-1H-pyrazole-5-carboxylic acid (812.4 mg, 80.8 mol, 1.20 eq) and was purified by prep-TLC (SiO₂, DCM:MeOH) to afford Compound 159, N-((1S)—((S)-3,3-difluorocyclohexyl)(6-(((5S)-2-oxo-5-(trifluoromethyl)piperidin-3-yl)methyl)imidazo[1,2-b]pyridazin-2-yl)methyl)-1-isopropyl-1H-pyrazole-5-carboxamide, (8.60 mg, 13.9 μmol, 21% yield) as a white solid. LCMS [M+H]⁺=582.3 m/z.

### Example 161: Preparation of Compound 160

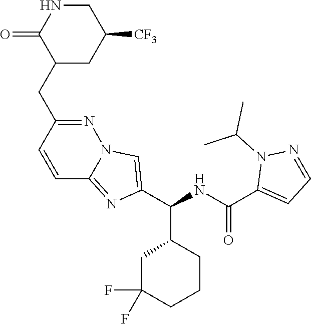 [SMILES: CC(C)n1nccc1C(=O)N[C@H](c1cn2nc(CC3C[C@H](C(F)(F)F)CNC3=O)ccc2n1)[C@@H]1CCCC(F)(F)C1]

N-((1S)—((R)-3,3-difluorocyclohexyl)(6-(((5S)-2-oxo-5-(trifluoromethyl)piperidin-3-yl)methyl)imidazo[1,2-b]pyridazin-2-yl)methyl)-1-isopropyl-1H-pyrazole-5-carboxamide was synthesized following the General Procedure 4 with (5S)-3-((2-((S)-amino((R)-3,3-difluorocyclohexyl)methyl)imidazo[1,2-b]pyridazin-6-yl)methyl)-5-(trifluoromethyl)piperidin-2-one (9.00 mg, 20.2 μmol, 1.00 eq), 1-isopropyl-1H-pyrazole-5-carboxylic acid (4.67 mg, 30.3 mol, 1.50 eq) and was purified by prep-HPLC (Phenomenex luna C18 250×50 mm, 15 um; mobile phase: [water (FA)-ACN]; B %: 41%-71%) to afford Compound 160, N-((1S)—((R)-3,3-difluorocyclohexyl)(6-(((5S)-2-oxo-5-(trifluoromethyl)piperidin-3-yl)methyl)imidazo[1,2-b]pyridazin-2-yl)methyl)-1-isopropyl-1H-pyrazole-5-carboxamide (5.20 mg, 8.91 μmol, 44% yield) as a white solid. LCMS [M+H]⁺=582.4 m/z.

### Example 162: Preparation of Compound 161

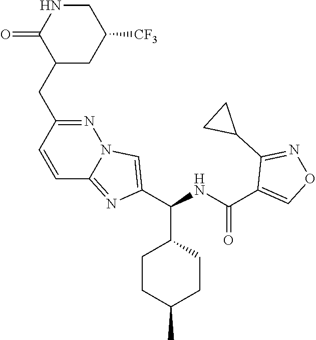 [SMILES: C[C@H]1CC[C@H]([C@H](NC(=O)c2conc2C2CC2)c2cn3nc(CC4C[C@@H](C(F)(F)F)CNC4=O)ccc3n2)CC1]

3-Cyclopropyl-N-((1S)—((1R.4S)-4-methylcyclohexyl)(6-(((5R)-2-oxo-5-(trifluoromethyl)piperidin-3-yl)methyl)imidazo[1,2-b]pyridazin-2-yl)methyl)isoxazole-4-carboxamide was synthesized following the General Procedure 4 with (5R)-3-((2-((S)-amino((1R.4S)-4-methylcyclohexyl)methyl)imidazo[1,2-b]pyridazin-6-yl)methyl)-5-(trifluoromethyl)piperidin-2-one (31 mg, 73 μmol, 1.00 eq), 3-cyclopropylisoxazole-4-carboxylic acid (25.2 mg, 165 μmol, 2.25 eq) and was purified by prep-HPLC (water (TFA)-acetonitrile (TFA)]; B %: 10%-60%) to afford Compound 161, 3-cyclopropyl-N-((1S)-((1R.4S)-4-methylcyclohexyl)(6-(((5R)-2-oxo-5-(trifluoromethyl)piperidin-3-yl)methyl)imidazo[1,2-b]pyridazin-2-yl)methyl)isoxazole-4-carboxamide, (36.7 mg, 64.3 μmol, 88% yield) as a white solid. LCMS [M+H]⁺=559.1 m/z.

### Example 163: Preparation of Compound 162

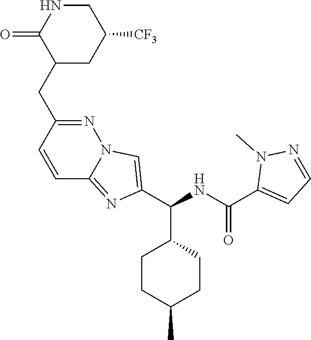 [SMILES: Cn1nccc1C(=O)N[C@H](c1cn2nc(CC3C[C@@H](C(F)(F)F)CNC3=O)ccc2n1)[C@H]1CC[C@H](C)CC1]

1-Methyl-N-((1S)—((1R.4S)-4-methylcyclohexyl)(6-(((5R)-2-oxo-5-(trifluoromethyl)piperidin-3-yl)methyl)imidazo[1,2-b]pyridazin-2-yl)methyl)-1H-pyrazole-5-carboxamide was synthesized following the General Procedure 4 with (5R)-3-((2-((S)-amino((1R.4S)-4-methylcyclohexyl)methyl)imidazo[1,2-b]pyridazin-6-yl)methyl)-5-(trifluoromethyl)piperidin-2-one (26.9 mg, 64 μmol, 1.00 eq), 1-methyl-1H-pyrazole-5-carboxylic acid (25.6 mg, 203 μmol, 3.20 eq) and was purified by prep-HPLC (water (TFA)-acetonitrile (TFA)]; B %: 10%-60%) to afford Compound 162, 1-methyl-N-((1S)-((1R.4S)-4-methylcyclohexyl)(6-(((5R)-2-oxo-5-(trifluoromethyl)piperidin-3-yl)methyl)imidazo[1,2-b]pyridazin-2-yl)methyl)-1H-pyrazole-5-carboxamide, (35 mg, 65.8 μmol, quantitative) as a white solid. LCMS [M+H]⁺=532.1 m/z.

### Example 164: Preparation of Compound 163

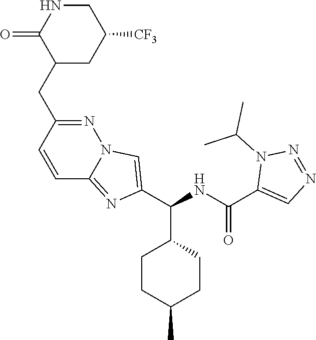 [SMILES: CC(C)n1nncc1C(=O)N[C@H](c1cn2nc(CC3C[C@@H](C(F)(F)F)CNC3=O)ccc2n1)[C@H]1CC[C@H](C)CC1]

N-((1S)—((1R.4S)-4-methylcyclohexyl)(6-(((5R)-2-oxo-5-(trifluoromethyl)piperidin-3-yl)methyl)imidazo[1,2-b]pyridazin-2-yl)methyl)-1-isopropyl-1H-1,2,3-triazole-5-carboxamide was synthesized following the General Procedure 4 with (5R)-3-((2-((S)-amino((1R.4S)-4-methylcyclohexyl)methyl)imidazo[1,2-b]pyridazin-6-yl)methyl)-5-(trifluoromethyl)piperidin-2-one (28.2 mg, 67 μmol, 1.00 eq), 1-isopropyl-1H-1,2,3-triazole-5-carboxylic acid (29.6 mg, 191 mol, 2.87 eq) and was purified by prep-HPLC (water (TFA)-acetonitrile (TFA)]; B %: 10%-60%) to afford Compound 163, N-((1S)-((1R.4S)-4-methylcyclohexyl)(6-(((5R)-2-oxo-5-(trifluoromethyl)piperidin-3-yl)methyl)imidazo[1,2-b]pyridazin-2-yl)methyl)-1-isopropyl-1H-1,2,3-triazole-5-carboxamide, (38 mg, 68.1 μmol, quantitative) as a white solid. LCMS [M+H]+=561.1 m/z.

### Example 165: Preparation of Compound 164

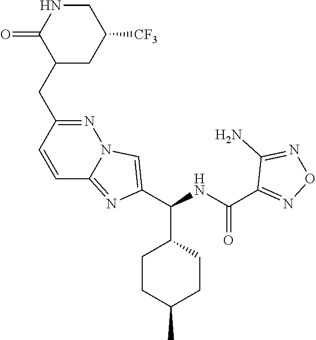 [SMILES: C[C@H]1CC[C@H]([C@H](NC(=O)c2nonc2N)c2cn3nc(CC4C[C@@H](C(F)(F)F)CNC4=O)ccc3n2)CC1]

4-Amino-N-((1S)—((1R.4S)-4-methylcyclohexyl)(6-(((5R)-2-oxo-5-(trifluoromethyl)piperidin-3-yl)methyl)imidazo[1,2-b]pyridazin-2-yl)methyl)-1,2,5-oxadiazole-3-carboxamide was synthesized following the General Procedure 4 with (5R)-3-((2-((S)-amino((1R.4S)-4-methylcyclohexyl)methyl)imidazo[1,2-b]pyridazin-6-yl)methyl)-5-(trifluoromethyl)piperidin-2-one (27.1 mg, 64 μmol, 1.00 eq), 4-amino-1,2,5-oxadiazole-3-carboxylic acid (29.6 mg, 229 μmol, 3.58 eq) and was purified by prep-HPLC (water (TFA)-acetonitrile (TFA)]; B %: 10%-60%) to afford Compound 164, 4-amino-N-((1S)—((1R.4S)-4-methylcyclohexyl)(6-(((5R)-2-oxo-5-(trifluoromethyl)piperidin-3-yl)methyl)imidazo[1,2-b]pyridazin-2-yl)methyl)-1,2,5-oxadiazole-3-carboxamide (33.2 mg, 62.1 μmol, 97% yield) as a white solid. LCMS [M+H]⁺=535.1 m/z.

### Example 166: Preparation of Compound 165

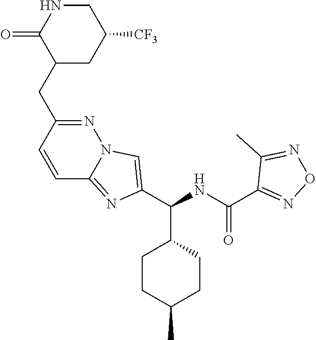 [SMILES: Cc1nonc1C(=O)N[C@H](c1cn2nc(CC3C[C@@H](C(F)(F)F)CNC3=O)ccc2n1)[C@H]1CC[C@H](C)CC1]

4-Methyl-N-((1S)-((1R.4S)-4-methylcyclohexyl)(6-(((5R)-2-oxo-5-(trifluoromethyl)piperidin-3-yl)methyl)imidazo[1,2-b]pyridazin-2-yl)methyl)-1,2,5-oxadiazole-3-carboxamide was synthesized following the General Procedure 4 with (5R)-3-((2-((S)-amino((1R.4S)-4-methylcyclohexyl)methyl)imidazo[1,2-b]pyridazin-6-yl)methyl)-5-(trifluoromethyl)piperidin-2-one (24.2 mg, 57 μmol, 1.00 eq), 4-methyl-1,2,5-oxadiazole-3-carboxylic acid (21.9 mg, 171 μmol, 3.00 eq) and was purified by prep-HPLC (water (TFA)-acetonitrile (TFA)]; B %: 10%-60%) to afford Compound 165, 4-methyl-N-((1S)—((1R.4S)-4-methylcyclohexyl)(6-(((5R)-2-oxo-5-(trifluoromethyl)piperidin-3-yl)methyl)imidazo[1,2-b]pyridazin-2-yl)methyl)-1,2,5-oxadiazole-3-carboxamide, (22.7 mg, 42.5 μmol, 75% yield) as a white solid. LCMS [M+H]⁺=534.1 m/z.

### Example 167: Preparation of Compound 166

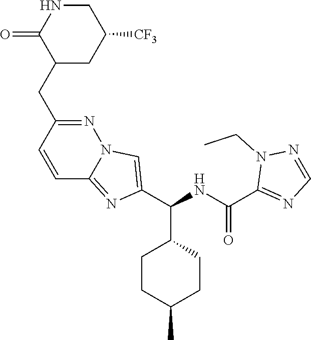 [SMILES: CCn1ncnc1C(=O)N[C@H](c1cn2nc(CC3C[C@@H](C(F)(F)F)CNC3=O)ccc2n1)[C@H]1CC[C@H](C)CC1]

N-((1S)-((1R.4S)-4-methylcyclohexyl)(6-(((5R)-2-oxo-5-(trifluoromethyl)piperidin-3-yl)methyl)imidazo[1,2-b]pyridazin-2-yl)methyl)-1-ethyl-1H-1,2,4-triazole-5-carboxamide was synthesized following the General Procedure 4 with (5R)-3-((2-((S)-amino((1R.4S)-4-methylcyclohexyl)methyl)imidazo[1,2-b]pyridazin-6-yl)methyl)-5-(trifluoromethyl)piperidin-2-one (15 mg, 35 μmol, 1.00 eq), 1-ethyl-1H-1,2,4-triazole-5-carboxylic acid (16.0 mg, 113 μmol, 3.20 eq) and was purified by prep-HPLC (water (TFA)-acetonitrile (TFA)]; B %: 10%-60%) to afford Compound 166, N-((1S)-((1R.4S)-4-methylcyclohexyl)(6-(((5R)-2-oxo-5-(trifluoromethyl)piperidin-3-yl)methyl)imidazo[1,2-b]pyridazin-2-yl)methyl)-1-ethyl-1H-1,2,4-triazole-5-carboxamide, (5 mg, 9.2 μmol, 26% yield) as a yellow solid. LCMS [M+H]⁺=547.6 m/z.

### Example 168: Preparation of Compound 167

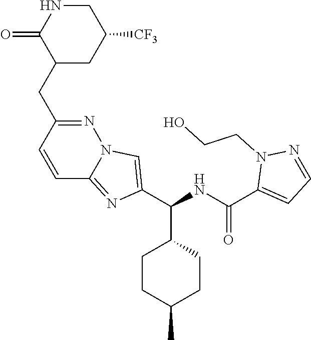 [SMILES: C[C@H]1CC[C@H]([C@H](NC(=O)c2ccnn2CCO)c2cn3nc(CC4C[C@@H](C(F)(F)F)CNC4=O)ccc3n2)CC1]

1-(2-Hydroxyethyl)-N—((S)-((1R.4S)-4-methylcyclohexyl)(6-(((3R.5R)-2-oxo-5-(trifluoromethyl)piperidin-3-yl)methyl)imidazo[1,2-b]pyridazin-2-yl)methyl)-1H-pyrazole-5-carboxamide was synthesized following the General Procedure 4 with (5R)-3-((2-((S)-amino((1R.4S)-4-methylcyclohexyl)methyl)imidazo[1,2-b]pyridazin-6-yl)methyl)-5-(trifluoromethyl)piperidin-2-one (5 mg, 12 μmol, 1.00 eq), 1-(2-hydroxyethyl)-1H-pyrazole-5-carboxylic acid (3.3 mg, 21 μmol, 1.80 eq) and was purified by prep-HPLC [(water (TFA)-acetonitrile (TFA)]; B %: 10%-60%) to afford Compound 167, 1-(2-hydroxyethyl)-N—((S)-((1R.4S)-4-methylcyclohexyl)(6-(((3R.5R)-2-oxo-5-(trifluoromethyl)piperidin-3-yl)methyl)imidazo[1,2-b]pyridazin-2-yl)methyl)-1H-pyrazole-5-carboxamide, (0.45 mg, 0.8 mol, 7% yield) as a white solid. LCMS [M+H]⁺=562.3 m/z.

### Example 169: Preparation of Compound 168

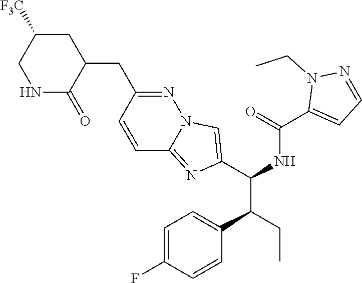 [SMILES: CC[C@@H](c1ccc(F)cc1)[C@H](NC(=O)c1ccnn1CC)c1cn2nc(CC3C[C@@H](C(F)(F)F)CNC3=O)ccc2n1]

1-Ethyl-N-((1S,2S)-2-(4-fluorophenyl)-1-(6-(((3R.5R)-2-oxo-5-(trifluoromethyl)piperidin-3-yl)methyl)imidazo[1,2-b]pyridazin-2-yl)butyl)-1H-pyrazole-5-carboxamide was synthesized following the General Procedure 4 with (3R.5R)-3-((2-((1S,2S)-1-amino-2-(4-fluorophenyl)butyl)imidazo[1,2-b]pyridazin-6-yl)methyl)-5-(trifluoromethyl)piperidin-2-one (20.0 mg, 43.2 μmol, 1.00 eq), 1-ethyl-1H-pyrazole-5-carboxylic acid (9.07 mg, 64.7 μmol, 1.50 eq) and was purified by prep-HPLC (Phenomenex luna C18 150×25 mm, 10 μm; mobile phase: [water (FA)-ACN]; B %: 40%-70%) to afford Compound 168, 1-ethyl-N-((1S,2S)-2-(4-fluorophenyl)-1-(6-(((3R.5R)-2-oxo-5-(trifluoromethyl)piperidin-3-yl)methyl)imidazo[1,2-b]pyridazin-2-yl)butyl)-1H-pyrazole-5-carboxamide, (21.1 mg, 34.7 μmol, 81% yield) as a white solid. LCMS [M+H]⁺=586.3 m/z.

### Example 170: Preparation of Compound 169

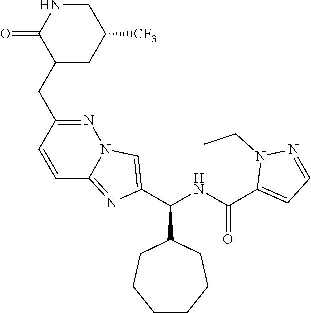 [SMILES: CCn1nccc1C(=O)N[C@H](c1cn2nc(CC3C[C@@H](C(F)(F)F)CNC3=O)ccc2n1)C1CCCCCC1]

N—((S)-cycloheptyl(6-(((3R.5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (3R.5R)-3-((2-((S)-amino(cycloheptyl)methyl)imidazo[1,2-b]pyridazin-6-yl)methyl)-5-(trifluoromethyl)piperidin-2-one (40.0 mg, 83.0 μmol, 1.00 eq), 1-ethyl-1H-pyrazole-5-carboxylic acid (13.9 mg, 99.6 μmol, 1.20 eq) and was purified by prep-TLC (SiO₂, DCM:MeOH) to afford Compound 169, N—((S)-cycloheptyl(6-(((3R.5R)-2-oxo-5-(trifluoromethyl)piperidin-3-yl)methyl)imidazo[1,2-b]pyridazin-2-yl)methyl)-1-ethyl-1H-pyrazole-5-carboxamide, (45.0 mg, 74.5 μmol, 90% yield) as a white solid. LCMS [M+H]⁺=546.3 m/z.

### Example 171: Preparation of Compound 170

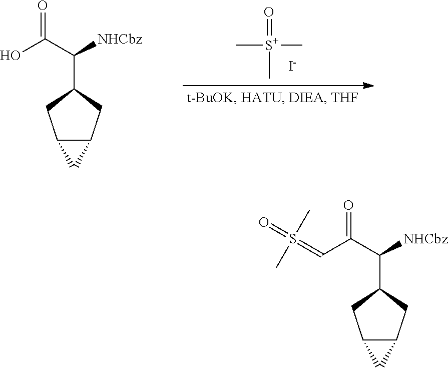

The preparation of mixture A: trimethylsulfoxonium iodide (912 mg, 4.15 mmol, 3.00 eq) was added to a solution of t-BuOK (465 mg, 4.15 mmol, 3.00 eq) in THF (5.0 mL) and the reaction mixture was stirred at 65° C. for 2 h under N₂. After that time, the solution was cooled to 0° C. to obtain the mixture A.

The preparation of mixture B: To a solution of (S)-2-(((benzyloxy)carbonyl)amino)-2-((1R,3S,5S)-bicyclo[3.1.0]hexan-3-yl)acetic acid (400 mg, 1.38 mmol, 1.00 eq) in THF (2.0 mL) was added DIEA (181 mg, 1.80 mmol, 250 μL, 1.30 eq) and HATU (683 mg, 1.80 mmol, 1.30 eq) at 25° C. The reaction mixture was stirred at 25° C. for 2 h. The suspension was then filtered, and the filtrate was cooled to 0° C. to obtain the mixture B.

The mixture B was added to the mixture A at 0° C. The resulting mixture was stirred at 25° C. for 2 h. The reaction mixture was quenched by the addition of water at 25° C., and extracted with DCM. The combined organic layers were dried over anhydrous Na₂SO₄, filtered and concentrated under reduced pressure to give a residue, which was purified by Prep-TLC (SiO₂, DCM:MeOH=10:1) to afford benzyl ((S)-1-((1R,3S,5S)-bicyclo[3.1.0]hexan-3-yl)-3-(dimethyl(oxo)-l⁶-sulfaneylidene)-2-oxopropyl)carbamate (190 mg, 522 μmol, 38% yield) as a yellow solid. LCMS [M+H]⁺=364.2 m/z.

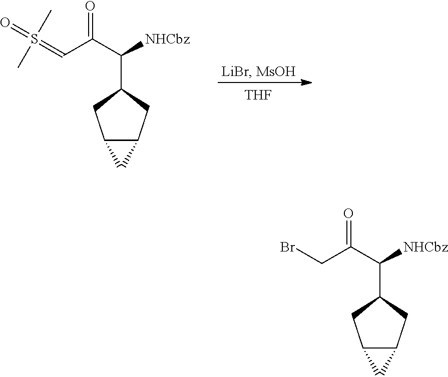 [SMILES: CS(C)(=O)=CC(=O)[C@@H](NC(=O)OCc1ccccc1)[C@@H]1C[C@H]2C[C@H]2C1 ; O=C(N[C@H](C(=O)CBr)[C@@H]1C[C@H]2C[C@H]2C1)OCc1ccccc1 ; "THF"]

To a solution of benzyl ((S)-1-((1R,3S,5S)-bicyclo[3.1.0]hexan-3-yl)-3-(dimethyl(oxo)-l⁶-sulfaneylidene)-2-oxopropyl)carbamate (190 mg, 522 μmol, 1.00 eq) in THE (3.00 mL) was added LiBr (68.1 mg, 784 μmol, 19.6 μL, 1.50 eq) and methane sulfonic acid (75.3 mg, 784 μmol, 56.0 L, 1.50 eq). The reaction mixture was stirred at 45° C. for 1 h. The reaction mixture was diluted with water and extracted with DCM. The combined organic layers were dried over anhydrous Na₂SO₄, filtered and concentrated under reduced pressure to afford benzyl ((S)-1-((1R,3S,5S)-bicyclo[3.1.0]hexan-3-yl)-3-bromo-2-oxopropyl)carbamate (190 mg, 518 μmol, 99% yield) as a yellow solid.

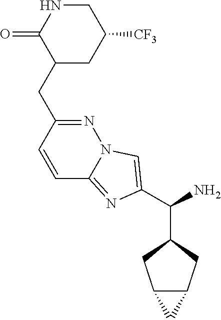 [SMILES: N[C@H](c1cn2nc(CC3C[C@@H](C(F)(F)F)CNC3=O)ccc2n1)[C@@H]1C[C@H]2C[C@H]2C1]

(5R)-3-((2-((S)-amino((1R,3S,5S)-bicyclo[3.1.0]hexan-3-yl)methyl)imidazo[1,2-b]pyridazin-6-yl)methyl)-5-(trifluoromethyl)piperidin-2-one was synthesized according to General Procedure 1, employing (5R)-3-((6-aminopyridazin-3-yl)methyl)-5-(trifluoromethyl)piperidin-2-one and benzyl ((S)-1-((1R,3S,5S)-bicyclo[3.1.0]hexan-3-yl)-3-bromo-2-oxopropyl)carbamate, and General Procedure 3, employing benzyl ((1S)-((1R,3S,5S)-bicyclo[3.1.0]hexan-3-yl)(6-(((5R)-2-oxo-5-(trifluoromethyl)piperidin-3-yl)methyl)imidazo[1,2-b]pyridazin-2-yl)methyl)carbamate.

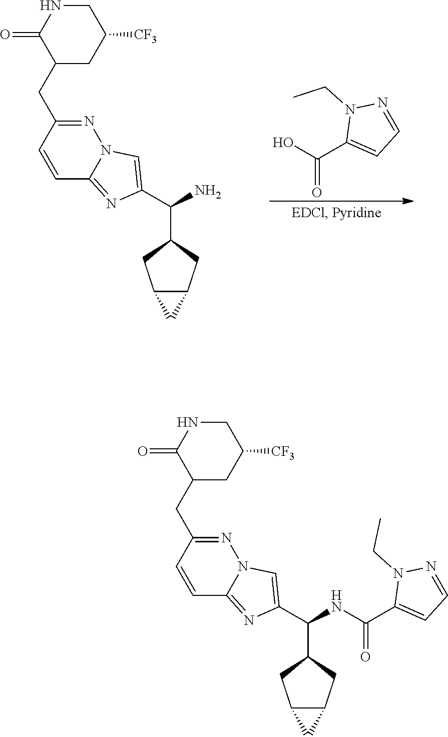

N-((1S)-((1R,3S,5S)-bicyclo[3.1.0]hexan-3-yl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5R)-3-((2-((S)-amino((1R,3S,5S)-bicyclo[3.1.0]hexan-3-yl)methyl)imidazo[1,2-b]pyridazin-6-yl)methyl)-5-(trifluoromethyl)piperidin-2-one (30.0 mg, 73.6 μmol, 1.00 eq), 1-ethyl-1H-pyrazole-5-carboxylic acid (12.3 mg, 88.3 μmol, 1.20 eq) and was purified by prep-TLC (SiO₂, DCM:MeOH) to afford Compound 170, N-((1S)—((1R,3S,5S)-bicyclo[3.1.0]hexan-3-yl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, (18.6 mg, 33.7 μmol, 46% yield) as a white solid. LCMS [M+H]⁺=530.3 m/z.

### Example 172: Preparation of Compound 171

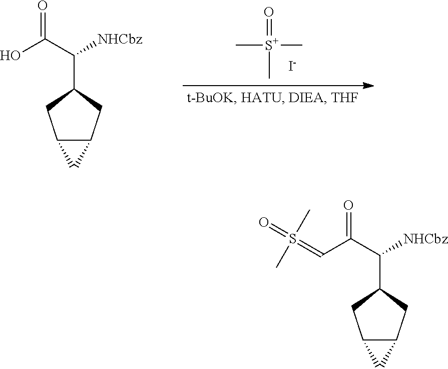

The preparation of mixture A: trimethylsulfoxonium iodide (2.28 g, 10.4 mmol, 3.00 eq) was added to a solution of t-BuOK (1.16 g, 10.4 mmol, 3.00 eq) in THF (15.0 mL) and the reaction mixture was stirred at 65° C. for 2 h under N₂. After that time, the solution was cooled to 0° C. to obtain the mixture A.

The preparation of mixture B: To a solution of (R)-2-(((benzyloxy)carbonyl)amino)-2-((IR, 3S,5S)-bicyclo[3.1.0]hexan-3-yl)acetic acid (1.00 g, 3.46 mmol, 1.00 eq) in THE (10.0 mL) was added DIEA (525 mg, 5.18 mmol, 722 μL, 1.50 eq) and HATU (1.97 g, 5.18 mmol, 1.50 eq) at 0° C. The reaction mixture was stirred at 25° C. for 2 h. The suspension was then filtered, and the filtrate was cooled to 0° C. to obtain the mixture B.

The mixture B was added to the mixture A at 0° C. The resulting mixture was stirred at 0° C. for 2 h. The reaction mixture was quenched by the addition of water at 25° C., and extracted with ethyl acetate. The combined organic layers were dried over anhydrous Na₂SO₄, filtered and concentrated under reduced pressure to give a residue, which was purified by column chromatography (SiO₂, DCM:MeOH=10:1) to afford benzyl ((R)-1-((1R,3S,5S)-bicyclo[3.1.0]hexan-3-yl)-3-(dimethyl(oxo)-l⁶-sulfaneylidene)-2-oxopropyl)carbamate (50 mg, 1.24 mmol, 36% yield) as a yellow oil. LCMS [M+H]⁺=364.3 m/z.

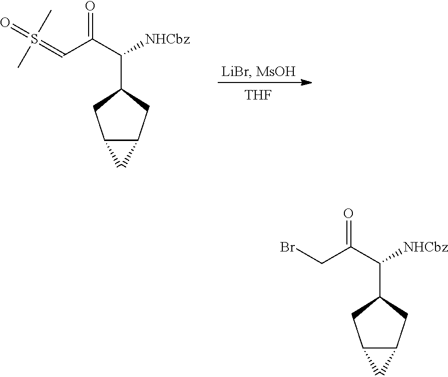 [SMILES: CS(C)(=O)=CC(=O)[C@H](NC(=O)OCc1ccccc1)[C@@H]1C[C@H]2C[C@H]2C1 ; O=C(N[C@@H](C(=O)CBr)[C@@H]1C[C@H]2C[C@H]2C1)OCc1ccccc1 ; "THF"]

To a solution of benzyl ((R)-1-((1R,3S,5S)-bicyclo[3.1.0]hexan-3-yl)-3-(dimethyl(oxo)-l⁶-sulfaneylidene)-2-oxopropyl)carbamate (450 mg, 1.24 mmol, 1.00 eq) in THE (8.00 mL) was added LiBr (323 mg, 3.71 mmol, 93.2 μL, 3.00 eq) and methane sulfonic acid (357 mg, 3.71 mmol, 265 μL, 3.00 eq). The reaction mixture was stirred at 45° C. for 2 h. The reaction mixture was diluted with water and extracted with ethyl acetate. The combined organic layers were dried over anhydrous Na₂SO₄, filtered and concentrated under reduced pressure to afford benzyl ((R)-1-((1R,3S,5S)-bicyclo[3.1.0]hexan-3-yl)-3-bromo-2-oxopropyl)carbamate (400 mg, 1.09 mmol, 88% yield) as a yellow oil.

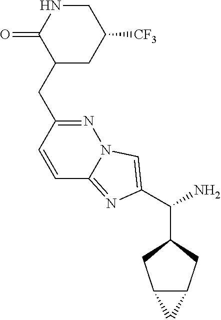 [SMILES: N[C@@H](c1cn2nc(CC3C[C@@H](C(F)(F)F)CNC3=O)ccc2n1)[C@@H]1C[C@H]2C[C@H]2C1]

(5R)-3-((2-((R)-amino((1R,3S,5S)-bicyclo[3.1.0]hexan-3-yl)methyl)imidazo[1,2-b]pyridazin-6-yl)methyl)-5-(trifluoromethyl)piperidin-2-one was synthesized according to General Procedure 1, employing (5R)-3-((6-aminopyridazin-3-yl)methyl)-5-(trifluoromethyl)piperidin-2-one and benzyl ((R)-1-((1R,3S,5S)-bicyclo[3.1.0]hexan-3-yl)-3-bromo-2-oxopropyl)carbamate, and General Procedure 3, employing benzyl ((1R)-((1R,3S,5S)-bicyclo[3.1.0]hexan-3-yl)(6-(((5R)-2-oxo-5-(trifluoromethyl)piperidin-3-yl)methyl)imidazo[1,2-b]pyridazin-2-yl)methyl)carbamate.

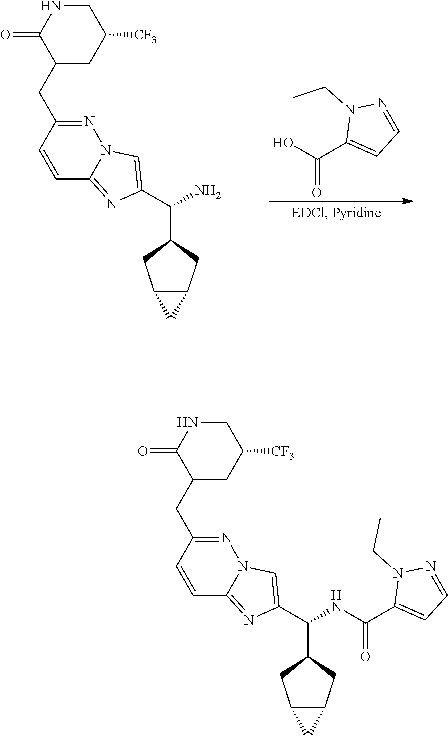

N—((1R)—((1R,3S,5S)-bicyclo[3.1.0]hexan-3-yl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5R)-3-((2-((R)-amino((1R,3S,5S)-bicyclo[3.1.0]hexan-3-yl)methyl)imidazo[1,2-b]pyridazin-6-yl)methyl)-5-(trifluoromethyl)piperidin-2-one (45.0 mg, 110 μmol, 1.00 eq), 1-ethyl-1H-pyrazole-5-carboxylic acid (17.0 mg, 121 μmol, 1.10 eq) and was purified by prep-TLC (SiO₂, DCM:MeOH) to afford Compound 171, N-((1R)-((1R,3S,5S)-bicyclo[3.1.0]hexan-3-yl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (35.32 mg, 64.6 μmol, 59% yield) as a white solid. LCMS [M+H]⁺=530.3 m/z.

### Example 173: Preparation of Compound 172

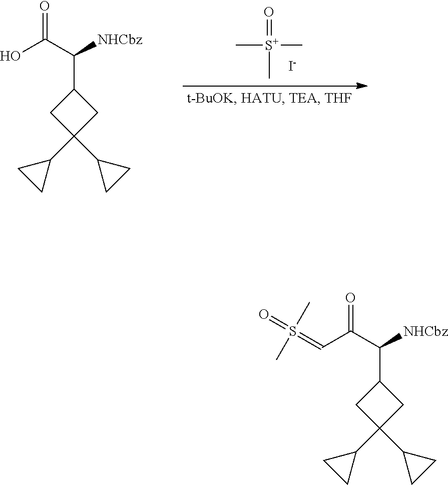

The preparation of mixture A: trimethylsulfoxonium iodide (961 mg, 4.37 mmol, 3.00 eq) was added to a solution of t-BuOK (490 mg, 4.37 mmol, 3.00 eq) in THF (10.0 mL) and the reaction mixture was stirred at 65° C. for 2 h under N₂. After that time, the solution was cooled to 0° C. to obtain the mixture A.

The preparation of mixture B: To a solution of (S)-2-(((benzyloxy)carbonyl)amino)-2-(3,3-dicyclopropylcyclobutyl)acetic acid (500 mg, 1.46 mmol, 1.00 eq) in THE (10.0 mL) was added TEA (192 mg, 1.89 mmol, 263 μL, 1.30 eq) and HATU (720 mg, 1.89 mmol, 1.30 eq) at 25° C. The reaction mixture was stirred at 25° C. for 2 h. The suspension was then filtered, and the filtrate was cooled to 0° C. to obtain the mixture B.

The mixture B was added to the mixture A at 0° C. The resulting mixture was stirred at 25° C. for 1 h. The reaction mixture was quenched by the addition of water at 25° C., and extracted with ethyl acetate. The combined organic layers were dried over anhydrous Na₂SO₄, filtered and concentrated under reduced pressure to give a residue, which was purified by prep-TLC (SiO₂, DCM:MeOH=10:1) to afford benzyl (S)-(1-(3,3-dicyclopropylcyclobutyl)-3-(dimethyl(oxo)-l⁶-sulfaneylidene)-2-oxopropyl)carbamate (530 mg, 1.27 mmol, 87% yield) as a yellow oil. LCMS [M+H]⁺=418.2 m/z.

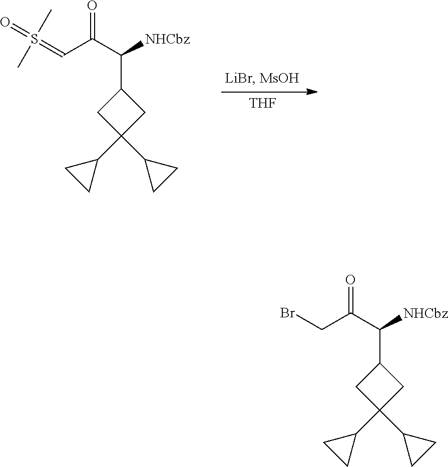 [SMILES: CS(C)(=O)=CC(=O)[C@@H](NC(=O)OCc1ccccc1)C1CC(C2CC2)(C2CC2)C1 ; O=C(N[C@H](C(=O)CBr)C1CC(C2CC2)(C2CC2)C1)OCc1ccccc1 ; "THF"]

To a solution of benzyl (S)-(1-(3,3-dicyclopropylcyclobutyl)-3-(dimethyl(oxo)-l⁶-sulfaneylidene)-2-oxopropyl)carbamate (530 mg, 1.27 mmol, 1.00 eq) in THE (10.0 mL) was added LiBr (165 mg, 1.90 mmol, 47.8 μL, 1.50 eq) and methane sulfonic acid (183 mg, 1.90 mmol, 136 μL, 1.50 eq). The reaction mixture was stirred at 45° C. for 4 h. The reaction mixture was diluted with water and extracted with ethyl acetate. The combined organic layers were dried over anhydrous Na₂SO₄, filtered and concentrated under reduced pressure to afford benzyl (S)-(3-bromo-1-(3,3-dicyclopropylcyclobutyl)-2-oxopropyl)carbamate (600 mg, crude) as a yellow oil.

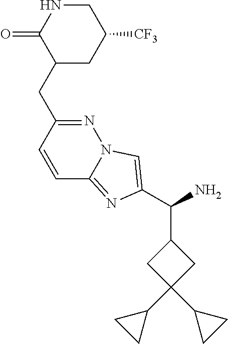 [SMILES: N[C@H](c1cn2nc(CC3C[C@@H](C(F)(F)F)CNC3=O)ccc2n1)C1CC(C2CC2)(C2CC2)C1]

(5R)-3-((2-((S)-amino(3,3-dicyclopropylcyclobutyl)methyl)imidazo[1,2-b]pyridazin-6-yl)methyl)-5-(trifluoromethyl)piperidin-2-one was synthesized according to General Procedure 1, employing (5R)-3-((6-aminopyridazin-3-yl)methyl)-5-(trifluoromethyl)piperidin-2-one and benzyl (S)-(3-bromo-1-(3,3-dicyclopropylcyclobutyl)-2-oxopropyl)carbamate, and General Procedure 3, employing benzyl ((1S)-(3,3-dicyclopropylcyclobutyl)(6-(((5R)-2-oxo-5-(trifluoromethyl)piperidin-3-yl)methyl)imidazo[1,2-b]pyridazin-2-yl)methyl)carbamate.

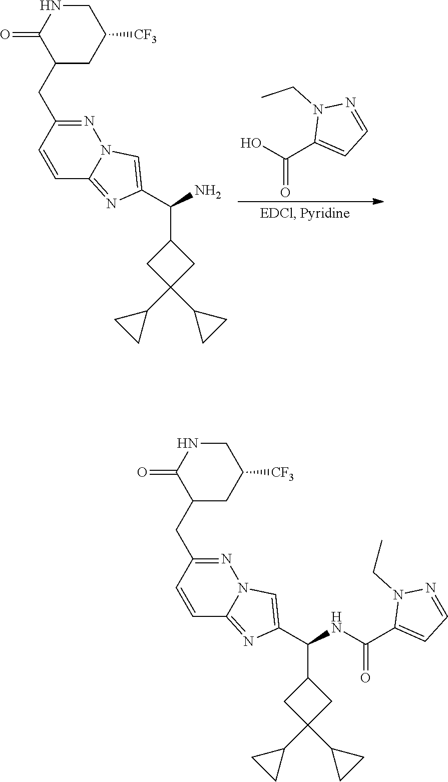

N-((1S)-(3,3-dicyclopropylcyclobutyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5R)-3-((2-((S)-amino(3,3-dicyclopropylcyclobutyl)methyl)imidazo[1,2-b]pyridazin-6-yl)methyl)-5-(trifluoromethyl)piperidin-2-one (52.0 mg, 113 μmol, 1.00 eq), 1-ethyl-1H-pyrazole-5-carboxylic acid (47.4 mg, 338 μmol, 3.00 eq) and was purified by prep-TLC (SiO₂, DCM:MeOH) and SFC (column: DAICEL CHIRALPAK IG 250 mm×30 mm, 5 um; mobile phase: [CO₂—EtOH (0.1% NH₃H₂O)]; B %: 40%) to afford Compound 172, N-((1S)-(3,3-dicyclopropylcyclobutyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, (32.01 mg, 54.7 μmol, 49% yield) as a yellow solid. LCMS [M+H]⁺=584.3 m/z.

### Example 174: Preparation of Compound 173

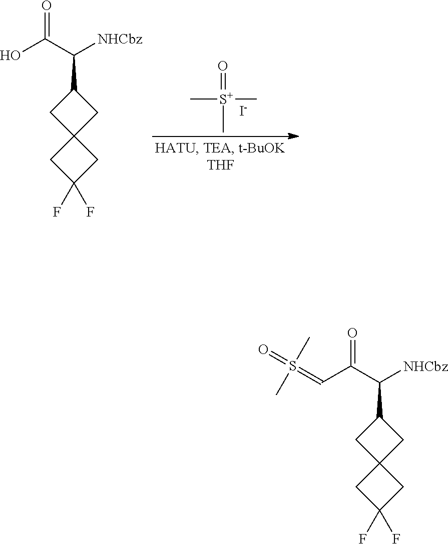

The preparation of mixture A: trimethylsulfoxonium iodide (700 mg, 3.18 mmol, 3.00 eq) was added to a solution of t-BuOK (357 mg, 3.18 mmol, 3.00 eq) in THF (3.0 mL) and the resulting mixture was stirred at 65° C. for 1 h under N₂. After that time, the reaction mixture was cooled to 0° C. to obtain the mixture A.

The preparation of mixture B: To a solution of (S)-2-(((benzyloxy)carbonyl)amino)-2-(6,6-difluorospiro[3.3]heptan-2-yl)acetic acid (0.360 g, 1.06 mmol, 1.00 eq) in THF (3.0 mL) was added HATU (605 mg, 1.59 mmol, 1.50 eq) and TEA (161 mg, 1.59 mmol, 222 μL, 1.50 eq) at 0° C. The reaction mixture was stirred at 0° C. for 1 h. The suspension was then filtered and the filtrate was cooled to 0° C. to obtain the mixture B.

The mixture B was added to the mixture A at 0° C. The resulting mixture was stirred at 0° C. for 0.5 h. The reaction mixture was filtered and concentrated under reduced pressure to give a residue, which was purified by prep-TLC (SiO₂, PE:EtOAc=1:1 to 0:1) to afford benzyl (S)-(1-(6,6-difluorospiro[3.3]heptan-2-yl)-3-(dimethyl(oxo)-l⁶-sulfaneylidene)-2-oxopropyl)carbamate (260 mg, 629 μmol, 59% yield) as a yellow oil.

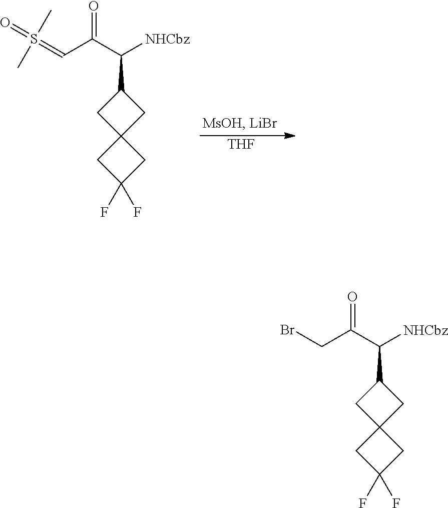 [SMILES: CS(C)(=O)=CC(=O)[C@@H](NC(=O)OCc1ccccc1)C1CC2(C1)CC(F)(F)C2 ; O=C(N[C@H](C(=O)CBr)C1CC2(C1)CC(F)(F)C2)OCc1ccccc1 ; "THF"]

To a solution of benzyl (S)-(1-(6,6-difluorospiro[3.3]heptan-2-yl)-3-(dimethyl(oxo)-l⁶-sulfaneylidene)-2-oxopropyl)carbamate (0.250 g, 605 μmol, 1.00 eq) in THF (3.00 mL) was added LiBr (78.8 mg, 907 μmol, 22.8 μL, 1.50 eq) and methane sulfonic acid (87.2 mg, 907 μmol, 64.8 L, 1.50 eq). The reaction mixture was stirred at 45° C. for 1 h. The reaction mixture was diluted with water and extracted with ethyl acetate. The combined organic layers were dried over anhydrous Na₂SO₄, filtered and concentrated under reduced pressure to afford benzyl (S)-(3-bromo-1-(6,6-difluorospiro[3.3]heptan-2-yl)-2-oxopropyl)carbamate (250 mg, 601 μmol, 99% yield) as a yellow oil.

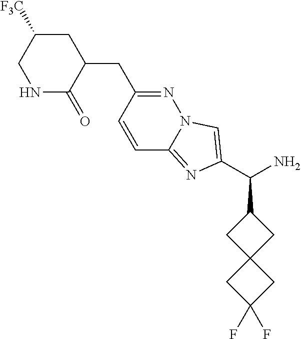 [SMILES: N[C@H](c1cn2nc(CC3C[C@@H](C(F)(F)F)CNC3=O)ccc2n1)C1CC2(C1)CC(F)(F)C2]

(5R)-3-((2-((S)-amino(6,6-difluorospiro[3.3]heptan-2-yl)methyl)imidazo[1,2-b]pyridazin-6-yl)methyl)-5-(trifluoromethyl)piperidin-2-one was synthesized according to General Procedure 1, employing (5R)-3-((6-aminopyridazin-3-yl)methyl)-5-(trifluoromethyl)piperidin-2-one and benzyl (S)-(3-bromo-1-(6,6-difluorospiro[3.3]heptan-2-yl)-2-oxopropyl)carbamate, and General Procedure 2, employing benzyl ((1S)-(6,6-difluorospiro[3.3]heptan-2-yl)(6-(((5R)-2-oxo-5-(trifluoromethyl)piperidin-3-yl)methyl)imidazo[1,2-b]pyridazin-2-yl)methyl)carbamate.

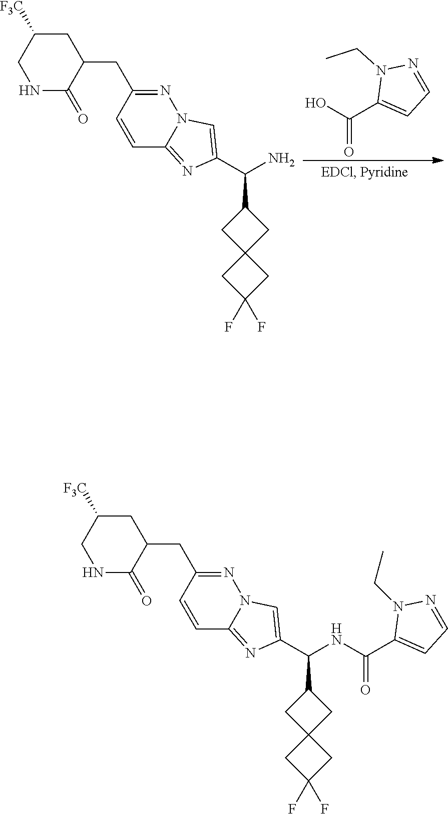

N-((1S)-(6,6-difluorospiro[3.3]heptan-2-yl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5R)-3-((2-((S)-amino(6,6-difluorospiro[3.3]heptan-2-yl)methyl)imidazo[1,2-b]pyridazin-6-yl)methyl)-5-(trifluoromethyl)piperidin-2-one (50.0 mg, 109 μmol, 1.00 eq), 1-ethyl-1H-pyrazole-5-carboxylic acid (22.9 mg, 163 μmol, 1.50 eq) and was purified by prep-TLC (SiO₂, DCM:MeOH) to afford N-((1S)-(6,6-difluorospiro[3.3]heptan-2-yl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (20.0 mg, 32.5 μmol, 30% yield) as a white solid. LCMS [M+H]⁺=580.3 m/z.

The title compound Compound 173 was isolated as the first eluting, single stereoisomer by chiral SFC purification (column: DAICEL CHIRALPAK AD (250 mm×30 mm, 10 μm); mobilephase: [CO₂—EtOH (0.1% NH₃H₂O)]; B %: 40%-−40%); (8.96 mg, 14.7 μmol, 43% yield) and was obtained as a yellow solid. LCMS [M+H]⁺=580.3 m/z.

### Example 175: Preparation of Compounds 174, 175, and 176

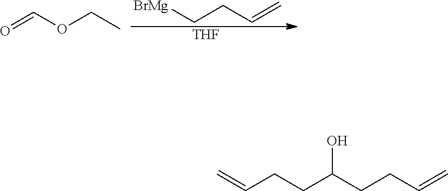

To a solution of ethyl formate (15.0 g×8, 202 mmol×8, 16.3 mL×8, 1.00 eq) in THE (200 mL) was added dropwise but-3-en-1-ylmagnesium bromide (1.00 M, 486 mL, 2.40 eq) at 0° C. The reaction mixture was stirred at 25° C. for 12 h. The reaction mixture was poured into ice a solution of ammonium chloride then extracted with ethyl acetate, the combined organic phase was dried over anhydrous Na₂SO₄ and concentrated under reduced pressure to give a residue, which was purified by column chromatography (SiO₂, PE:EtOAc=100:1 to 3:1) to afford nona-1,8-dien-5-ol (130 g, 927 mmol, 57% yield) as a yellow oil. ¹H NMR (400 MHz, CDCl₃) δ 5.89-5.82 (m, 2H), 5.06 (dd, J₁=17.2 Hz, J₂=1.6 Hz, 2H), 4.99 (d, J=10.0 Hz, 2H), 3.70-3.64 (m, 1H), 2.23-2.14 (m, 4H), 1.60-1.53 (m, 4H).

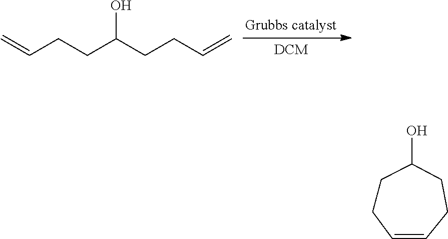

To a solution of nona-1,8-dien-5-ol (15.0 g×4, 107 mmol×4, 1.00 eq) in DCM (1.50 L) was added Grubbs catalyst (4.41 g, 5.35 mmol, 0.0500 eq). The reaction mixture was stirred at 45° C. for 5 h. The reaction mixture was concentrated under reduced pressure to give a residue, which was purified by column chromatography (SiO₂, PE:EtOAc=100:1 to 3:1) to afford cyclohept-4-en-1-ol (30.0 g, 267 mmol, 63% yield) as a yellow oil. ¹H NMR (400 MHz, CDCl₃) δ 5.83-5.79 (m, 2H), 3.89-3.86 (m, 1H), 2.25-2.23 (m, 2H), 1.99-1.94 (m, 4H), 1.48-1.45 (m, 2H).

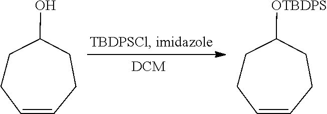

To a solution of cyclohept-4-en-1-ol (45.0 g, 401 mmol, 1.00 eq) in DCM (500 mL) was added imidazole (54.6 g, 802 mmol, 2.00 eq) and TBDPSCl (116 g, 421 mmol, 108 mL, 1.05 eq) at 0° C. The reaction mixture was stirred at 25° C. for 2 h. The reaction mixture was diluted with water and extracted with DCM. The combined organic phase was washed with brine, dried over anhydrous Na₂SO₄ and concentrated under reduced pressure to give a residue, which was purified by column chromatography (SiO₂, PE:EtOAc=100:1 to 5:1) to afford tert-butyl(cyclohept-4-en-1-yloxy)diphenylsilane (130 g, 371 mmol, 92% yield) as a light yellow oil. ¹H NMR (400 MHz, CDCl₃) δ 7.70-7.68 (m, 4H), 7.43-7.36 (m, 6H), 5.79-5.72 (m, 2H), 4.00-3.95 (m, 1H), 2.36-2.33 (m, 2H), 1.82-1.78 (m, 2H), 1.69-1.66 (m, 2H), 1.63-1.60 (m, 2H), 1.08 (s, 9H). (bicyclo[5.1.0]octan-4-yloxy)(tert-butyl)diphenylsilane

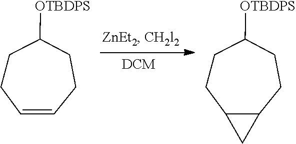

To a solution of tert-butyl(cyclohept-4-en-1-yloxy)diphenylsilane (40.0 g×2, 114 mmol×2, 1.00 eq) in DCM (400 mL) was added dropwise diodomethane (122 g, 456 mmol, 36.8 mL, 4.00 eq) and diethyl zinc (1.00 M, 456 mL, 4.00 eq) at 0° C. The reaction mixture was stirred at 25° C. for 12 h. The reaction mixture was poured into ice saturated solution of ammonium chloride, then filtered. The resulting filter was washed with brine and extracted with DCM, dried over anhydrous Na₂SO₄ and concentrated under reduced pressure to give a residue, which was purified by column chromatography (SiO₂, PE:EtOAc=1:0 to 10:1) to afford (bicyclo[5.1.0]octan-4-yloxy)(tert-butyl)diphenylsilane (75.0 g, 206 mmol, 90% yield) as a yellow oil. ¹H NMR (400 MHz, CDCl₃) δ 7.68-7.64 (m, 4H), 7.42-7.35 (m, 6H), 4.15-3.47 (m, 1H), 1.90-1.85 (m, 3H), 1.77-1.71 (m, 1H), 1.61-1.58 (m, 1H), 1.45-1.42 (m, 1H), 1.05 (s, 9H), 0.91-0.87 (m, 1H), 0.77-0.54 (m, 4H), 0.15-−0.20 (m, 1H).

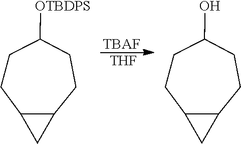

To a solution of (bicyclo[5.1.0]octan-4-yloxy)(tert-butyl)diphenylsilane (45.0 g, 123 mmol, 1.00 eq) in THE (300 mL) was added TBAF (1.00 M, 370 mL, 3.00 eq). The reaction mixture was stirred at 50° C. for 8 h. The reaction mixture was diluted with water and extracted with ethyl acetate. The combined organic phase was washed with brine, dried over anhydrous Na₂SO₄ and concentrated under reduced pressure to give a residue, which was purified by column chromatography (SiO₂, PE:EtOAc=100:1 to 3:1) to afford bicyclo[5.1.0]octan-4-ol (29.0 g) as a yellow oil. ¹H NMR (400 MHz, CDCl₃) δ 4.16-3.46 (m, 1H), 2.10-2.05 (m, 1H), 2.02-1.96 (m, 1H), 1.84-1.67 (m, 3H), 1.49-1.42 (m, 1H), 1.39-1.27 (m, 1H), 0.82-0.64 (m, 4H), 0.13-0.09 (m, 1H)

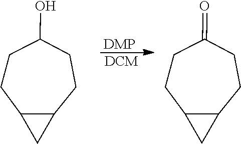

To a solution of bicyclo[5.1.0]octan-4-ol (15.0 g, 119 mmol, 1.00 eq) in DCM (150 mL) was added DMP (60.5 g, 143 mmol, 44.2 mL, 1.20 eq) at 0° C. The reaction mixture was stirred at 25° C. for 2 h. The reaction mixture was diluted with a aqueous solution of saturated sodium sulfite at 0° C. and extracted with DCM. The combined organic phase was washed with brine, dried over anhydrous Na₂SO₄ and concentrated under reduced pressure to give a residue, which was purified by column chromatography (SiO₂, PE:EtOAc=1:0 to 3:1) to afford bicyclo[5.1.0]octan-4-one (12.0 g, 96.6 mmol, 81% yield) as a yellow oil. ¹H NMR (400 MHz, CDCl₃) δ 2.53-2.50 (m, 4H), 2.27-2.22 (m, 2H), 1.10-1.04 (m, 4H), 0.77-0.75 (m, 1H), 0.08-0.05 (m, 1H).

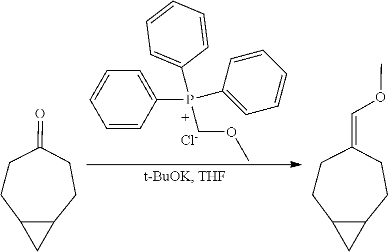

To a solution of (methoxymethyl)triphenylphosphonium chloride (33.1 g, 96.6 mmol, 1.20 eq, HCl salt) in THF (100 mL) was added dropwise t-BuOK (1.00 M, 96.6 mL, 1.20 eq) at 0° C. The reaction mixture was stirred at 0° C. for 1 h, then bicyclo[5.1.0]octan-4-one (10.0 g, 80.5 mmol, 1.00 eq) in THE (20.0 mL) was added. The reaction mixture was stirred at 25° C. for 3 h. The reaction mixture was diluted with water and extracted with ethyl acetate. The combined organic phase was washed with brine, dried over anhydrous Na₂SO₄ and concentrated under reduced pressure to give a residue, which was purified by column chromatography (SiO₂, PE:EtOAc=1:0 to 5:1) to afford 4-(methoxymethylene)bicyclo[5.1.0]octane (8.50 g, 55.8 mmol, 69% yield) as a yellow oil. ¹H NMR (400 MHz, CDCl₃) δ 5.72 (s, 1H), 3.52 (s, 3H), 2.82 (dd, J1=13.6 Hz, J2=6.8 Hz, 1H), 2.25-2.20 (m, 2H), 2.09-1.99 (m, 2H), 1.75-1.72 (m, 1H), 0.91-0.83 (m, 4H), 0.69-0.67 (m, 1H), 0.04-0.01 (m, 1H).

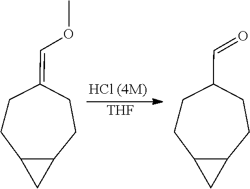

To a solution of 4-(methoxymethylene)bicyclo[5.1.0]octane (4.50 g, 29.6 mmol, 1.00 eq) in THE (40.0 mL) was added HCl (4.00 M, 11.1 mL, 1.50 eq) at 0° C. The reaction mixture was stirred at 25° C. for 0.5 h. An aqueous solution of saturated NaCO₃ was added to adjust pH to 8, then extracted with ethyl acetate. The combined organic phase was washed with brine, dried over anhydrous Na₂SO₄ and concentrated under reduced pressure to afford bicyclo[5.1.0]octane-4-carbaldehyde (4.00 g, crude) as a yellow oil. ¹H NMR (400 MHz, CDCl₃) δ 9.65-9.62 (m, 1H), 2.47-2.29 (s, 2H), 2.10-2.05 (m, 3H), 1.79-1.62 (m, 1H), 1.43-1.40 (m, 1H), 1.15-1.05 (m, 1H), 0.94-0.83 (m, 3H), 0.75-0.59 (m, 1H), 0.08-0.06 (m, 1H).

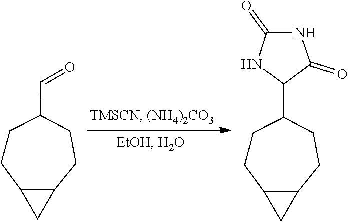 [SMILES: O=CC1CCC2CC2CC1 ; O=C1NC(=O)C(C2CCC3CC3CC2)N1]

To a solution of bicyclo[5.1.0]octane-4-carbaldehyde (4.00 g, 28.9 mmol, 1.00 eq) in EtOH (30.0 mL) and water (30.0 mL) was added ammonium carbonate (11.4 g, 145 mmol, 11.9 mL, 5.00 eq) and TMSCN (3.59 g, 36.2 mmol, 4.53 mL, 1.25 eq). The reaction mixture was stirred at 90° C. for 12 h. Water was added to the reaction mixture, then filtered and the filter cake was washed with water and DCM, then the filter cake was dried under reduced pressure to afford 5-(bicyclo[5.1.0]octan-4-yl)imidazolidine-2,4-dione (2.50 g, crude) as a yellow solid. ¹H NMR (400 MHz, DMSO-d₆) δ 10.56 (s, 1H), 7.90-7.86 (m, 1H), 3.92 (d, J=2.4 Hz, 1H), 2.11-2.07 (m, 1H), 1.66-1.63 (m, 3H), 1.40-1.31 (m, 4H), 0.90-0.76 (m, 3H), 0.61-0.28 (m, 1H), 0.22-−0.01 (m, 1H).

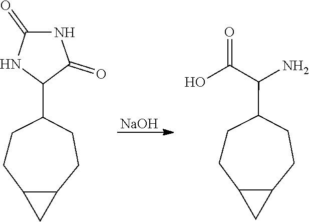

A solution of 5-(bicyclo[5.1.0]octan-4-yl)imidazolidine-2,4-dione (2.50 g, 12.0 mmol, 1.00 eq) in NaOH (2.00 M, 25.0 mL, 4.17 eq) was stirred at 120° C. for 12 h. HCl (1.00 M) was added to adjust pH to 9 and concentrated under reduced pressure to afford 2-amino-2-(bicyclo[5.1.0]octan-4-yl)acetic acid (2.20 g, crude) as a yellow liquid. LCMS [M+H]⁺=184.5 m/z

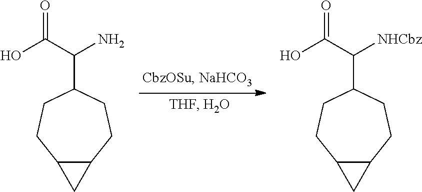 [SMILES: NC(C(=O)O)C1CCC2CC2CC1 ; O=C(NC(C(=O)O)C1CCC2CC2CC1)OCc1ccccc1]

To a solution of 2-amino-2-(bicyclo[5.1.0]octan-4-yl)acetic acid (2.20 g, 12.0 mmol, 1.00 eq) in THF (25.0 mL) and water (25.0 mL) was added NaCO₃ (2.02 g, 24.0 mmol, 934 μL, 2.00 eq) and CbzOSu (3.59 g, 14.4 mmol, 1.20 eq). The reaction mixture was stirred at 25° C. for 1 h. HCl (1.00 M) was added to adjust pH to 5, then extracted with ethyl acetate. The combined organic phase was dried over anhydrous Na₂SO₄ and concentrated under reduced pressure to give a residue, which was purified by Prep-HPLC (column: Phenomenexluna C18 250×80 mm, 10 μm; mobile phase: [water (FA)-ACN]; gradient: 40%-70% B) to afford 2-(((benzyloxy)carbonyl)amino)-2-(bicyclo[5.1.0]octan-4-yl)acetic acid (2.50 g, 7.88 mmol, 66% yield) as a white solid. ¹H NMR (400 MHz, DMSO-d₆) δ 12.56 (s, 1H), 7.51-7.45 (m, 1H), 7.38-7.31 (m, 5H), 5.03-5.02 (m, 2H), 4.01-3.96 (m, 1H), 2.11-2.07 (m, 1H), 1.72-1.65 (m, 3H), 1.41-1.24 (m, 4H), 0.86-0.75 (m, 3H), 0.60-0.32 (m, 1H), 0.23-−0.02 (m, 1H).

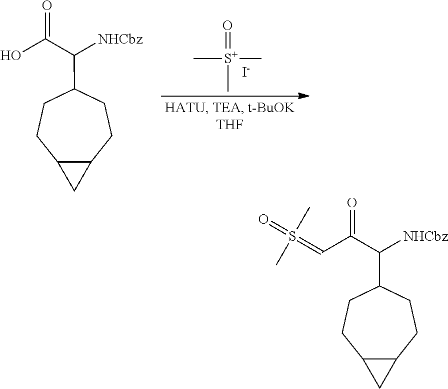

The preparation of mixture A: trimethylsulfoxonium iodide (2.08 g, 9.45 mmol, 3.00 eq) was added to a solution of t-BuOK (1.00 M, 9.45 mL, 3.00 eq) in THE (20.0 mL) and the resulting mixture was stirred at 65° C. for 2 h under N₂. After that time, the reaction mixture was cooled to 0° C. to obtain the mixture A.

The preparation of mixture B: To a solution of 2-(((benzyloxy)carbonyl)amino)-2-(bicyclo[5.1.0]octan-4-yl)acetic acid (1.00 g, 3.15 mmol, 1.00 eq) in THF (20.0 mL) was added HATU (1.56 g, 4.10 mmol, 1.30 eq) and TEA (414 mg, 4.10 mmol, 570 μL, 1.30 eq) at 25° C. The reaction mixture was stirred at 25° C. for 2 h. The suspension was then filtered and the filtrate was cooled to 0° C. to obtain the mixture B.

The mixture B was added to the mixture A at 0° C. The resulting mixture was stirred at 25° C. for 1.5 h. The reaction mixture was filtered and concentrated under reduced pressure to give a residue, which was purified by Prep-HPLC (column: Kromasil Eternity XT 250×80 mm, 10 μm; mobile phase: [water (NH₄HCO₃)-ACN]; gradient: 33%-63% B) to afford benzyl (1-(bicyclo[5.1.0]octan-4-yl)-3-(dimethyl(oxo)-l⁶-sulfaneylidene)-2-oxopropyl)carbamate (1.10 g, 2.81 mmol, 89% yield) as a yellow solid. ¹H NMR (400 MHz, CDCl3) δ 0.38-7.32 (m, 5H), 5.56-5.43 (m, 1H), 5.11-5.10 (m, 2H), 4.50 (s, 1H), 4.10-4.07 (m, 1H), 3.41-3.37 (m, 6H), 2.18-2.17 (m, 1H), 1.80-1.62 (m, 4H), 1.33-1.30 (m, 1H), 0.88-0.78 (m, 4H), 0.65-0.37 (m, 1H), 0.19-−0.04 (m, 1H).

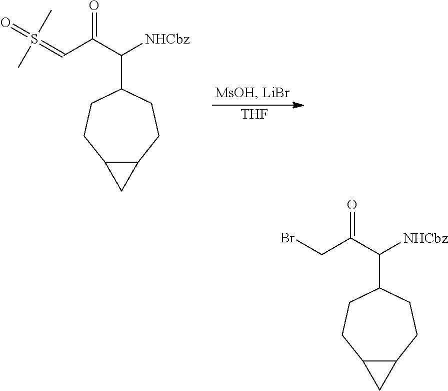 [SMILES: CS(C)(=O)=CC(=O)C(NC(=O)OCc1ccccc1)C1CCC2CC2CC1 ; O=C(NC(C(=O)CBr)C1CCC2CC2CC1)OCc1ccccc1 ; "THF"]

To a solution of benzyl(1-(bicyclo[5.1.0]octan-4-yl)-3-(dimethyl(oxo)-l⁶-sulfaneylidene)-2-oxopropyl)carbamate (1.10 g, 2.81 mmol, 1.00 eq) in THF (20.00 mL) was added LiBr (732 mg, 8.43 mmol, 212 μL, 3.00 eq) and methane sulfonic acid (270 mg, 2.81 mmol, 201 μL, 1.00 eq). The reaction mixture was stirred at 45° C. for 1 h. The reaction mixture was diluted with water and extracted with ethyl acetate. The combined organic layers were dried over anhydrous Na₂SO₄, filtered and concentrated under reduced pressure to afford benzyl (1-(bicyclo[5.1.0]octan-4-yl)-3-bromo-2-oxopropyl)carbamate (1.10 g, crude) as a yellow solid. LCMS [M+H]⁺=394.0 m/z.

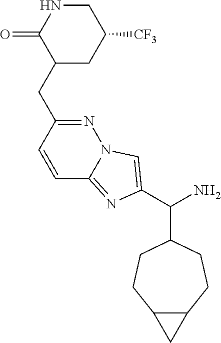 [SMILES: NC(c1cn2nc(CC3C[C@@H](C(F)(F)F)CNC3=O)ccc2n1)C1CCC2CC2CC1]

(5R)-3-((2-(amino(bicyclo[5.1.0]octan-4-yl)methyl)imidazo[1,2-b]pyridazin-6-yl)methyl)-5-(trifluoromethyl)piperidin-2-one was synthesized according to General Procedure 1, employing (5R)-3-((6-aminopyridazin-3-yl)methyl)-5-(trifluoromethyl)piperidin-2-one and benzyl (1-(bicyclo[5.1.0]octan-4-yl)-3-bromo-2-oxopropyl)carbamate, and General Procedure 3, employing benzyl (bicyclo[5.1.0]octan-4-yl(6-(((5R)-2-oxo-5-(trifluoromethyl)piperidin-3-yl)methyl)imidazo[1,2-b]pyridazin-2-yl)methyl)carbamate.

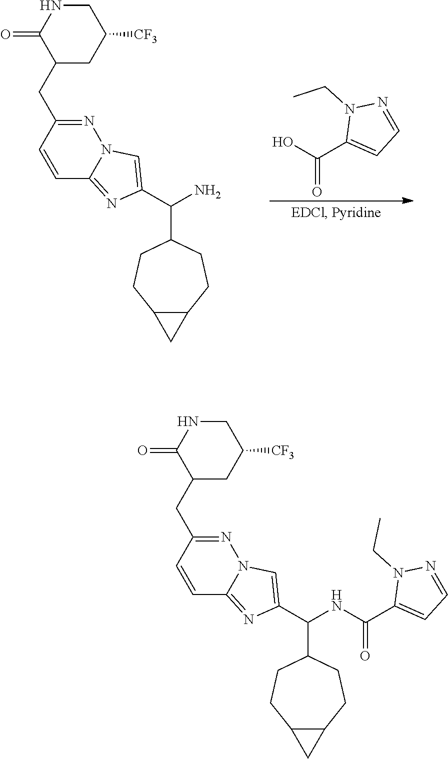

N-(bicyclo[5.1.0]octan-4-yl(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5R)-3-((2-(amino(bicyclo[5.1.0]octan-4-yl)methyl)imidazo[1,2-b]pyridazin-6-yl)methyl)-5-(trifluoromethyl)piperidin-2-one (360 mg, 826 μmol, 1.00 eq), 1-ethyl-1H-pyrazole-5-carboxylic acid (139 mg, 992 μmol, 1.20 eq) and was purified by prep-HPLC (column: PhenomenexLuna C18 150×25 mm, 10 μm; mobile phase: [water (FA)-ACN]; gradient: 40%-70% B) to afford N-(bicyclo[5.1.0]octan-4-yl(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (300 mg, 538 μmol, 65% yield) as a yellow solid. LCMS [M+H]⁺=558.4 m/z.

The title compound Compound 175 was isolated as the first eluting, single stereoisomer by chiral SFC purification (column: DAICEL CHIRALPAK AD-H (250 mm×30 mm, 5 um); mobile phase: [ethanol-isopropanol mixture]; B %: 30%); (31.63 mg, 56.5 μmol, 11% yield) and was obtained as a white solid. LCMS [M+H]⁺=558.4 m/z.

The title compound Compound 176 was isolated as the second eluting, single stereoisomer by chiral SFC purification (column: DAICEL CHIRALPAK AD-H (250 mm×30 mm, 5 um); mobile phase: [ethanol-isopropanol mixture]; B %: 300%); (96.94 mg, 173 μmol, 32% yield) and was obtained as a white solid. LCMS [M+H]⁺=558.4 m/z.

The title compound Compound 174 was isolated as the third eluting, single stereoisomer by chiral SFC purification (column: DAICEL CHIRALPAK AD-H (250 mm×30 mm, 5 um); mobile phase: [ethanol-isopropanol mixture]; B %: 30%); (39.03 mg, 65.8 μmol, 12% yield) and was obtained as a light yellow solid. LCMS [M+H]⁺=558.4 m/z.

### Example 176: Preparation of Compound 177

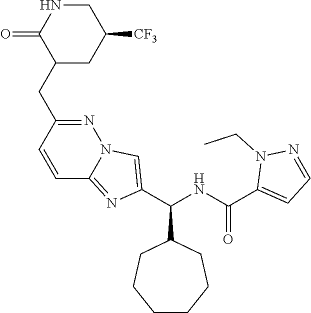 [SMILES: CCn1nccc1C(=O)N[C@H](c1cn2nc(CC3C[C@H](C(F)(F)F)CNC3=O)ccc2n1)C1CCCCCC1]

N—((S)-cycloheptyl(6-(((5S)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5S)-3-((2-((S)-amino(cycloheptyl)methyl)imidazo[1,2-b]pyridazin-6-yl)methyl)-5-(trifluoromethyl)piperidin-2-one (40.0 mg, 83.0 umol, 1.00 eq), 1-ethyl-1H-pyrazole-5-carboxylic acid (13.9 mg, 99.6 umol, 1.20 eq) and was purified by prep-TLC (SiO₂, DCM:MeOH) to afford Compound 177, N—((S)-cycloheptyl(6-(((5S)-2-oxo-5-(trifluoromethyl)piperidin-3-yl)methyl)imidazo[1,2-b]pyridazin-2-yl)methyl)-1-ethyl-1H-pyrazole-5-carboxamide, (45.0 mg, 74.5 μmol, 90% yield) as a yellow solid. LCMS [M+H]⁺=546.3 m/z.

### Example 177: Preparation of Intermediate 24

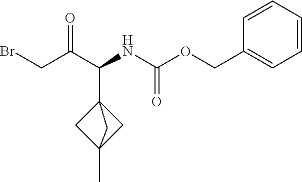 [SMILES: CC12CC([C@H](NC(=O)OCc3ccccc3)C(=O)CBr)(C1)C2]

Benzyl (S)-(3-bromo-1-(3-methylbicyclo[1.1.1]pentan-1-yl)-2-oxopropyl)carbamate (Intermediate 24)

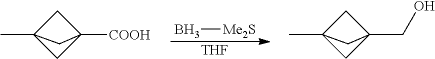

To a solution of 3-methylbicyclo[1.1.1]pentane-1-carboxylic acid (20.0 g, 158 mmol, 1.00 eq) in THF (200 mL) was added BH₃-Me₂S (12 M, 39.6 mL, 3.00 eq). The mixture was stirred at 25° C. for 2 h. The reaction mixture was quenched by addition of MeOH, and then concentrated under reduced pressure to give a residue to afford (3-methylbicyclo[1.1.1]pentan-1-yl) MeOH (11.0 g, 98.1 mmol, 62% yield) as a light yellow oil. ¹H NMR (400 MHz, CDCl₃) δ 3.56 (s, 2H), 1.54 (s, 6H), 1.28 (s, 3H).

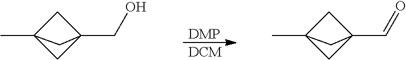

To a solution of (3-methylbicyclo[1.1.1]pentan-1-yl) MeOH (11.0 g, 98.1 mmol, 1.00 eq) in DCM (150 mL) was added DMP (54.1 g, 127 mmol, 39.5 mL, 1.30 eq). The reaction mixture was stirred at 25° C. for 2 h. The reaction mixture was quenched by the addition of a saturated aqueous solution of NaCO₃, and then diluted with DCM and extracted. The combined organic layers were washed with brine, dried over anhydrous Na₂SO₄, filtered and concentrated under reduced pressure to afford 3-methylbicyclo[1.1.1]pentane-1-carbaldehyde (13.0 g, crude) as a light yellow oil.

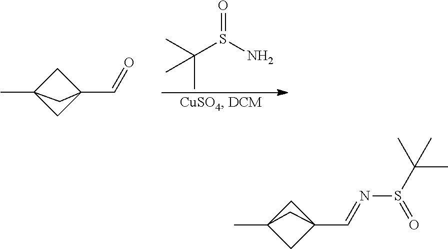

To a solution of 3-methylbicyclo[1.1.1]pentane-1-carbaldehyde (13.0 g, 118 mmol, 1.00 eq) in DCM (150 mL) was added CuSO4 (56.5 g, 354 mmol, 54.3 mL, 3.00 eq) and 2-methylpropane-2-sulfinamide (28.6 g, 236 mmol, 2.00 eq). The reaction mixture was stirred at 25° C. for 2 h. The reaction mixture was quenched by the addition of water and extracted with DCM The combined organic layers were washed with brine, dried over anhydrous Na₂SO₄, filtered and concentrated under reduced pressure to give a residue, which was purified by column chromatography (SiO₂, PE/EtOAc=10/1 to 3/1) to afford 2-methyl-N-((3-methylbicyclo[1.1.1]pentan-1-yl)methylene)propane-2-sulfinamide (13.0 g, 60.9 mmol, 52% yield) as alight-yellow oil. ¹H NMR (400 MHz, CDCl₃) δ 7.94 (s, 1H), 1.90 (s, 6H), 1.20 (s, 3H), 1.20 (s, 9H).

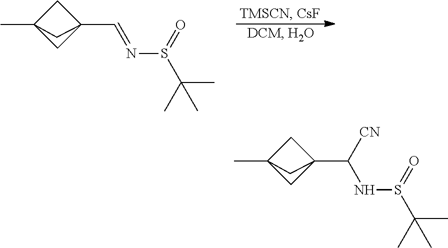

To a solution of 2-methyl-N-((3-methylbicyclo[1.1.1]pentan-1-yl)methylene)propane-2-sulfinamide (13.0 g, 60.9 mmol, 1.00 eq) in DCM (300 mL) was added TMSCN (9.07 g, 91.4 mmol, 11.4 mL, 1.50 eq), H₂O (2.50 g, 138 mmol, 2.50 mL, 2.28 eq) and CsF (4.63 g, 30.5 mmol, 1.12 mL, 0.50 eq). The reaction mixture was stirred at 25° C. for 2 h. The reaction mixture was diluted with water and extracted with DCM. The combined organic layers were washed with brine, dried over anhydrous Na₂SO₄, filtered and concentrated under reduced pressure to give a residue, which was purified by column chromatography (SiO₂, PE/EtOAc=5/1 to 1/1) to afford N-(cyano(3-methylbicyclo[1.1.1]pentan-1-yl)methyl)-2-methylpropane-2-sulfinamide (8.0 g, 33.3 mmol, 55% yield) as an off-white solid. ¹H NMR (400 MHz, CDCl₃) δ 4.20 (dd, J=8 Hz, 1H), 3.59 (dd, J=8 Hz, 1H), 1.71 (s, 6H), 1.23-1.21 (m, 12H).

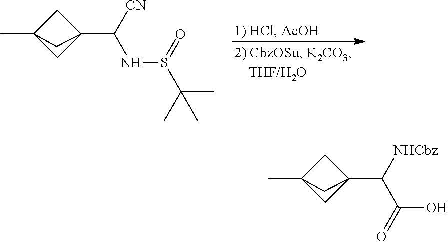

To a solution of N-(cyano(3-methylbicyclo[1.1.1]pentan-1-yl)methyl)-2-methylpropane-2-sulfinamide (8.0 g, 33.3 mmol, 1.00 eq) in AcOH (30 mL) was added HCl (12 M, 80.0 mL, 28.8 eq). The reaction mixture was stirred at 110° C. for 12 h. The reaction mixture was concentrated under reduced pressure to afford 2-amino-2-(3-methylbicyclo[1.1.1]pentan-1-yl)acetic acid (6.0 g, HCl salt) as an off-white solid. ¹H NMR (400 MHz, CDCl₃) δ 4.05 (s, 1H), 1.72 (s, 6H), 1.16 (s, 3H).

To a solution of 2-amino-2-(3-methylbicyclo[1.1.1]pentan-1-yl)acetic acid (6.0 g, crude, HCl) in THF (60 mL) and H₂O (60 mL) was added CbzOSu (15.0 g, 62.6 mmol) and K₂CO₃ (21.6 g, 156 mmol). The reaction mixture was stirred at 25° C. for 12 h. The reaction mixture was adjust to pH=4 with 1M HCl and diluted with EtOAc and extracted with EtOAc. The combined organic layers were washed with brine, dried over anhydrous Na₂SO₄, filtered and concentrated under reduced pressure to give a residue, which was purified by prep-HPLC (column: Phenomenex luna C18 (250×70 mm, 10 μm); mobile phase: [water (FA)-ACN]; B %: 35%-65%) to afford 2-(((benzyloxy)carbonyl)amino)-2-(3-methylbicyclo[1.1.1]pentan-1-yl)acetic acid (5.0 g, 17.3 mmol) as an off-white solid.

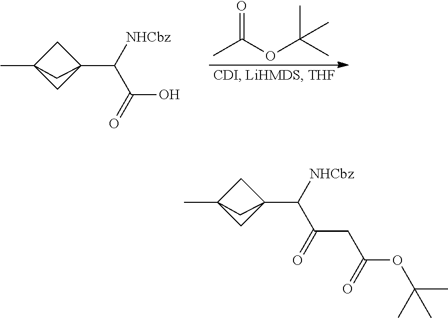

The reaction mixture 1: To a solution of 2-(((benzyloxy)carbonyl)amino)-2-(3-methylbicyclo[1.1.1]pentan-1-yl)acetic acid (5.00 g, 17.3 mmol, 1.00 eq) in THE (100 mL) was added CDI (3.36 g, 20.74 mmol, 1.20 eq). The mixture was stirred at 0° C. for 2 h.

The reaction mixture 2: To a solution of tert-butyl acetate (9.76 g, 84.0 mmol, 11.3 mL, 4.86 eq) in THF (100 mL) was added LDA (2 M, 42.0 mL, 4.86 eq). The mixture was stirred at −70° C. for 2 h.

The reaction mixture 1 was added to the reaction mixture 2, the mixture was stirred at −70° C. for another 2 h. The reaction mixture was quenched by the addition of water and then diluted with EtOAc and extracted with EtOAc. The combined organic layers were washed with brine, dried over anhydrous Na₂SO₄, filtered and concentrated under reduced pressure to give a residue, which was purified by column chromatography (SiO₂, PE/EtOAc=5/1 to 1/1) to afford tert-butyl 4-(((benzyloxy)carbonyl)amino)-4-(3-methylbicyclo[1.1.1]pentan-1-yl)-3-oxobutanoate (5.0 g, 12.9 mmol, 75% yield) as a light yellow oil.

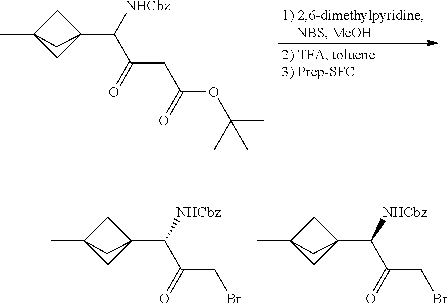

To a solution of tert-butyl 4-(((benzyloxy)carbonyl)amino)-4-(3-methylbicyclo[1.1.1]pentan-1-yl)-3-oxobutanoate (5.00 g, 12.9 mmol) in MeOH (70 mL) was added NBS (1.95 g, 11.0 mmol, 0.85 eq) and 2,6-dimethylpyridine (276 mg, 2.58 mmol, 300 μL, 0.20 eq). The reaction mixture was stirred at 0° C. for 2 h. The reaction mixture was concentrated under reduced pressure to afford tert-butyl 4-(((benzyloxy)carbonyl)amino)-2-bromo-4-(3-methylbicyclo[1.1.1]pentan-1-yl)-3-oxobutanoate (5.00 g, crude) as a light yellow oil.

To a solution of tert-butyl 4-(((benzyloxy)carbonyl)amino)-2-bromo-4-(3-methylbicyclo[1.1.1]pentan-1-yl)-3-oxobutanoate (5.0 g, 10.7 mmol, 1.00 eq) in toluene (50 mL) was added TFA (6.11 g, 53.6 mmol, 3.97 mL, 5.00 eq). The reaction mixture was stirred at 50° C. for 3 h. The reaction mixture was concentrated under reduced pressure to give a residue, which was purified by prep-HPLC (column: UniSil 10-120 C18 50×250 mm; mobile phase: [water(FA)-ACN]; B %: 50%-80%) to afford benzyl (3-bromo-1-(3-methylbicyclo[1.1.1]pentan-1-yl)-2-oxopropyl)carbamate (2.0 g, racemate) as a light yellow oil.

Benzyl (S)-(3-bromo-1-(3-methylbicyclo[1.1.1]pentan-1-yl)-2-oxopropyl)carbamate was isolated as the first eluting, single stereoisomer by chiral SFC purification (column: DAICEL CHIRALCEL OJ-H (250 mm×30 mm, Sum); mobile phase: [0.1% NH₃H₂O ETOH]; B %: 15%-15%); (773.4 mg, 2.12 mmol) and was obtained as a light yellow solid. ¹H NMR (400 MHz, CDCl₃) δ 7.42-7.34 (m, 5H), 5.32-5.30 (m, 1H), 5.13-5.09 (m, 2H), 4.72-4.69 (m, 1H), 4.06-3.97 (m, 2H), 1.63 (s, 6H), 1.19 (s, 3H). LCMS [M+H]⁺=366.1 m/z.

Benzyl (R)-(3-bromo-1-(3-methylbicyclo[1.1.1]pentan-1-yl)-2-oxopropyl)carbamate was isolated as the second eluting, single stereoisomer by chiral SFC purification (column: DAICEL CHIRALCEL OJ-H (250 mm×30 mm, Sum); mobile phase: [0.1% NH₃H₂O ETOH]; B %: 15%-15%); (694.6 mg, 1.90 mmol) and was obtained as a light yellow oil. ¹H NMR (400 MHz, CDCl₃) δ 7.39-7.30 (m, 5H), 5.32-5.29 (m, 1H), 5.13-5.09 (m, 2H), 4.72-4.69 (m, 1H), 4.06-3.97 (m, 2H), 1.67 (s, 6H), 1.17 (s, 3H).

### Example 178: Preparation of Compound 178

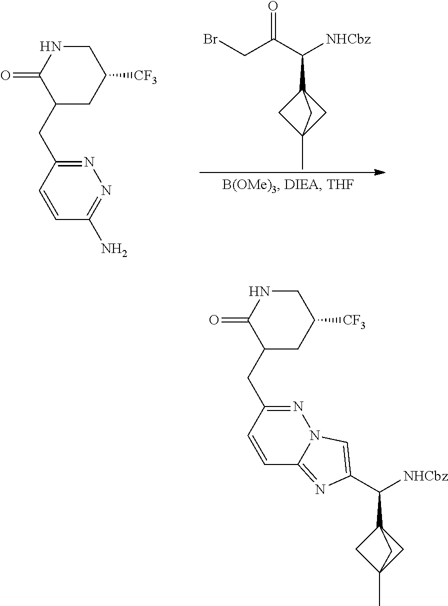 [SMILES: Nc1ccc(CC2C[C@@H](C(F)(F)F)CNC2=O)nn1 ; CC12CC([C@H](NC(=O)OCc3ccccc3)C(=O)CBr)(C1)C2 ; CC12CC([C@H](NC(=O)OCc3ccccc3)c3cn4nc(CC5C[C@@H](C(F)(F)F)CNC5=O)ccc4n3)(C1)C2]

Benzyl ((S)-(3-methylbicyclo[1.1.1]pentan-1-yl)(6-(((5R)-2-oxo-5-(trifluoromethyl)piperidin-3-yl)methyl)imidazo[1,2-b]pyridazin-2-yl)methyl)carbamate was synthesized following the General Procedure 1, using (5R)-3-((6-aminopyridazin-3-yl)methyl)-5-(trifluoromethyl)piperidin-2-one with benzyl (S)-(3-bromo-1-(3-methylbicyclo[1.1.1]pentan-1-yl)-2-oxopropyl)carbamate and was purified by prep-TLC (SiO₂, DCM:MeOH) to afford benzyl ((1S)-(3-methylbicyclo[1.1.1]pentan-1-yl)(6-(((5R)-2-oxo-5-(trifluoromethyl)piperidin-3-yl)methyl)imidazo[1,2-b]pyridazin-2-yl)methyl)carbamate (150 mg, 277 umol, 95% yield) as a yellow solid. ¹H NMR (400 MHz, CDCl₃) δ 7.85 (br d, J=8.8 Hz, 1H), 7.74 (s, 1H), 7.37-7.31 (m, 5H), 7.00 (br d, J=8.8 Hz, 1H), 5.80 (br d, J=1.2 Hz, 1H), 5.74-5.64 (m, 1H), 5.16-4.99 (m, 3H), 3.62-3.36 (m, 3H), 3.07 (dd, J1=15.2 Hz, J2=8.4 Hz, 1H), 2.98-2.85 (m, 1H), 2.77-2.65 (m, 1H), 2.32-2.21 (m, 1H), 1.84-1.77 (m, 1H), 1.57 (br s, 6H), 1.14 (s, 3H).

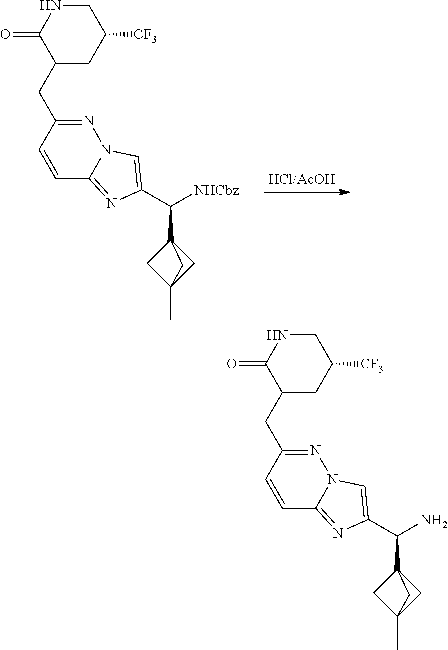 [SMILES: CC12CC([C@H](NC(=O)OCc3ccccc3)c3cn4nc(CC5C[C@@H](C(F)(F)F)CNC5=O)ccc4n3)(C1)C2 ; CC12CC([C@H](N)c3cn4nc(CC5C[C@@H](C(F)(F)F)CNC5=O)ccc4n3)(C1)C2]

To a solution of benzyl ((1S)-(3-methylbicyclo[1.1.1]pentan-1-yl)(6-(((5R)-2-oxo-5-(trifluoromethyl)piperidin-3-yl)methyl)imidazo[1,2-b]pyridazin-2-yl)methyl)carbamate (60.0 mg, 111 umol, 1.00 eq) in AcOH (0.100 mL) was added HCl (12.0 M, 1.00 mL, 108 eq). The reaction mixture was stirred at 60° C. for 0.5 h. The reaction mixture was cooled to 25° C., and then adjust pH=9 with saturated aqueous NaCO₃, then diluted with water and extracted with a mixture of DCM and MeOH. The combined organic layers were dried over anhydrous Na₂SO₄, filtered and concentrated under reduced pressure to afford (5R)-3-((2-((S)-amino(3-methylbicyclo[1.1.1]pentan-1-yl)methyl)imidazo[1,2-b]pyridazin-6-yl)methyl)-5-(trifluoromethyl)piperidin-2-one (45.0 mg, 110 μmol, quantitative) as a yellow oil. LCMS [M+H]⁺=408.1 m/z.

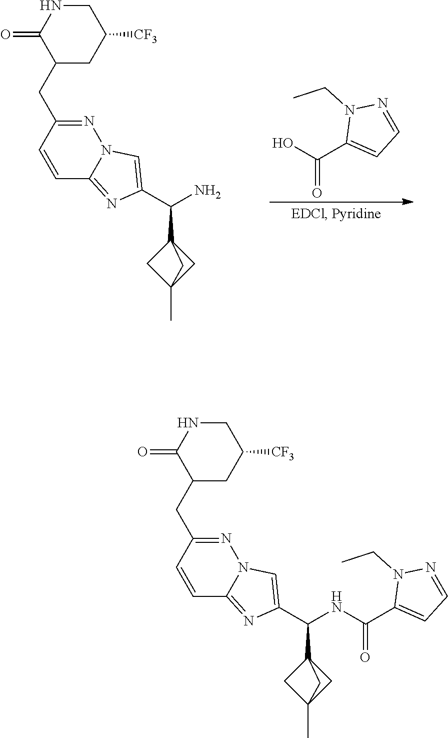

1-Ethyl-N-((1S)-(3-methylbicyclo[1.1.1]pentan-1-yl)(6-(((5R)-2-oxo-5-(trifluoromethyl)piperidin-3-yl)methyl)imidazo[1,2-b]pyridazin-2-yl)methyl)-1H-pyrazole-5-carboxamide was synthesized following the General Procedure 4 with (5R)-3-((2-((S)-amino(3-methylbicyclo[1.1.1]pentan-1-yl)methyl)imidazo[1,2-b]pyridazin-6-yl)methyl)-5-(trifluoromethyl)piperidin-2-one (40.0 mg, 98.2 umol, 1.00 eq), 1-ethyl-1H-pyrazole-5-carboxylic acid (41.3 mg, 295 umol, 3.00 eq) and was purified by prep-TLC (SiO₂, DCM:MeOH) to afford Compound 178, 1-ethyl-N-((1S)-(3-methylbicyclo[1.1.1]pentan-1-yl)(6-(((5R)-2-oxo-5-(trifluoromethyl)piperidin-3-yl)methyl)imidazo[1,2-b]pyridazin-2-yl)methyl)-1H-pyrazole-5-carboxamide, (35.2 mg, 65.8 umol, 67% yield) as a yellow solid. LCMS [M+H]⁺=530.2 m/z.

### Example 179: Preparation of Compound 179

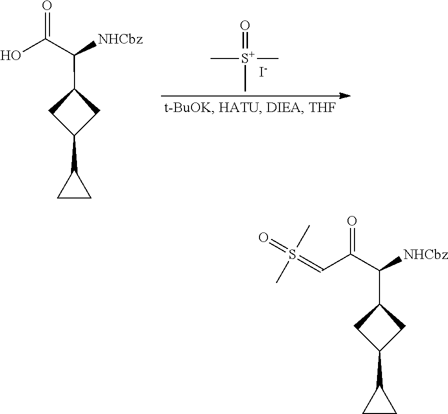

To a solution of trimethylsulfoxonium iodide (1.09 g, 4.94 mmol, 3.00 eq) in THF (10.0 mL) was added potassium tert-butoxide (554 mg, 4.94 mmol, 3.00 eq). The reaction mixture was stirred at 65° C. for 1 h (Reaction 1).

In parallel, to a solution of (S)-2-(((benzyloxy)carbonyl)amino)-2-((1S,3R)-3-cyclopropylcyclobutyl)acetic acid (500 mg, 1.65 mmol, 1.00 eq) in THE (10.0 mL) was added DIEA (250 mg, 2.47 mmol, 344 μL, 1.50 eq) followed by 1-[bis(dimethylamino)methylene]-1H-1,2,3-triazolo[4,5-b]pyridinium 3-oxide hexafluorophosphate (HATU, 940 mg, 2.47 mmol, 1.50 eq). The reaction mixture was stirred at 25° C. for 1 h (Reaction 2).

The content of the Reaction 2 was slowly added to the Reaction 1 at 0° C. and the resulting mixture was further stirred at 25° C. for 2 h. The reaction mixture was quenched by the addition of water and then diluted with the addition of ethyl acetate. An extraction was performed twice with DCM. The combined organic layers were washed with brine, dried over anhydrous Na₂SO₄, filtered and concentrated under reduced pressure to give a residue, which was purified by reversed-phase HPLC (column: Waters Xbridge C18 150×50 mm, 10 μm; mobile phase: [water (NH₄HCO₃)-ACN]; gradient: 29%-59% B) to afford benzyl ((S)-1-((1S,3R)-3-cyclopropylcyclobutyl)-3-(dimethyl(oxo)-l⁶-sulfaneylidene)-2-oxopropyl)carbamate 95.0 mg, 251 μmol, 15% yield) as a yellow solid. LCMS [M+H]+=378.2 m/z.

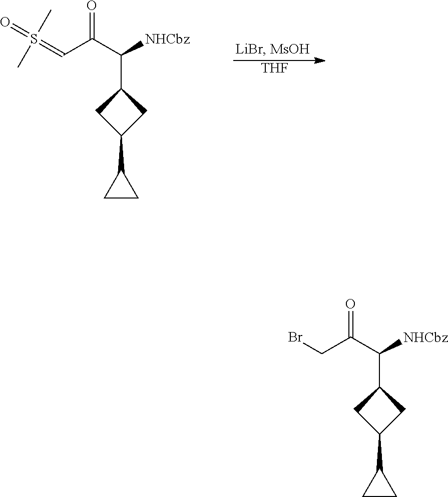 [SMILES: CS(C)(=O)=CC(=O)[C@@H](NC(=O)OCc1ccccc1)[C@H]1C[C@@H](C2CC2)C1 ; "THF" ; O=C(N[C@H](C(=O)CBr)[C@H]1C[C@@H](C2CC2)C1)OCc1ccccc1]

To a solution of benzyl ((S)-1-((1S,3R)-3-cyclopropylcyclobutyl)-3-(dimethyl(oxo)-l⁶-sulfaneylidene)-2-oxopropyl)carbamate (60.0 mg, 158 μmol, 1.00 eq) in THF (1.00 mL) was added LiBr (41.4 mg, 476 μmol, 11.9 μL, 3.00 eq) followed by methanesulfonic acid (15.2 mg 158 μmol, 11.3 μL, 1.00 eq). The reaction mixture was stirred at 45° C. for 1 h. The reaction mixture was quenched by the addition of water, and then extracted twice with DCM. The combined organic layers were washed with brine, dried over anhydrous Na₂SO₄, filtered and concentrated under reduced pressure to afford benzyl ((S)-3-bromo-1-((1S,3R)-3-cyclopropylcyclobutyl)-2-oxopropyl)carbamate (60.0 mg, 157 μmol, 99% yield) as a yellow solid.

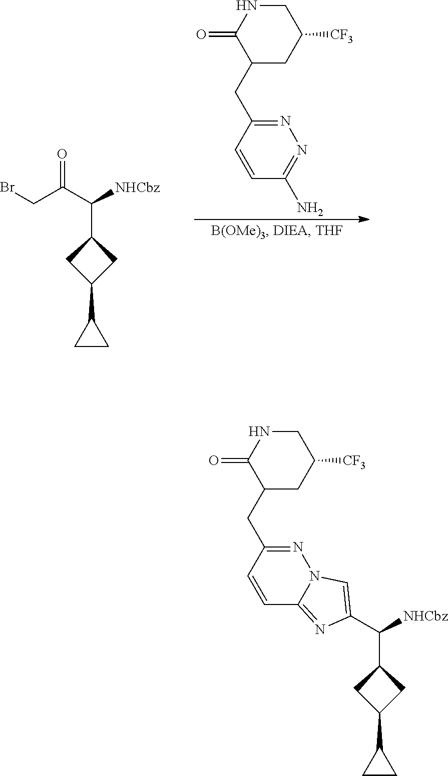 [SMILES: O=C(N[C@H](C(=O)CBr)[C@H]1C[C@@H](C2CC2)C1)OCc1ccccc1 ; Nc1ccc(CC2C[C@@H](C(F)(F)F)CNC2=O)nn1 ; O=C(N[C@H](c1cn2nc(CC3C[C@@H](C(F)(F)F)CNC3=O)ccc2n1)[C@H]1C[C@@H](C2CC2)C1)OCc1ccccc1]

Benzyl ((1S)-((1S,3R)-3-cyclopropylcyclobutyl)(6-(((5R)-2-oxo-5-(trifluoromethyl)piperidin-3-yl)methyl)imidazo[1,2-b]pyridazin-2-yl)methyl)carbamate was synthesized following the General Procedure 1, using (5R)-3-((6-aminopyridazin-3-yl)methyl)-5-(trifluoromethyl)piperidin-2-one with benzyl ((S)-3-bromo-1-((1S,3R)-3-cyclopropylcyclobutyl)-2-oxopropyl)carbamate and was purified by prep-TLC (SiO₂, DCM:MeOH) to afford benzyl ((1S)-((1S,3R)-3-cyclopropylcyclobutyl)(6-(((5R)-2-oxo-5-(trifluoromethyl)piperidin-3-yl)methyl)imidazo[1,2-b]pyridazin-2-yl)methyl)carbamate (65.0 mg, 116 μmol, 74% yield) as a white solid. LCMS [M+H]+=556.3 m/z.

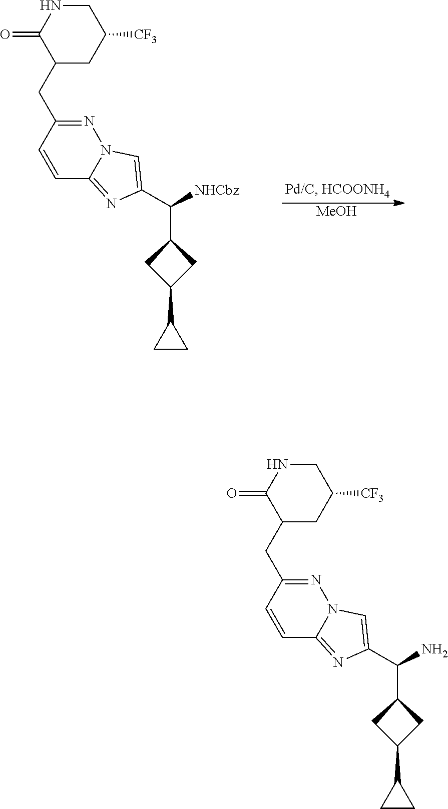 [SMILES: "MeOH" ; O=C(N[C@H](c1cn2nc(CC3C[C@@H](C(F)(F)F)CNC3=O)ccc2n1)[C@H]1C[C@@H](C2CC2)C1)OCc1ccccc1 ; N[C@H](c1cn2nc(CC3C[C@@H](C(F)(F)F)CNC3=O)ccc2n1)[C@H]1C[C@@H](C2CC2)C1]

To a solution of benzyl ((1S)-((1S,3R)-3-cyclopropylcyclobutyl)(6-(((5R)-2-oxo-5-(trifluoromethyl)piperidin-3-yl)methyl)imidazo[1,2-b]pyridazin-2-yl)methyl)carbamate (60.0 mg, 107 μmol, 1.00 eq) and HCOONH₄ (34.0 mg, 539 μmol, 5.00 eq) in MeOH (1.00 mL) was added Pd/C (15.0 mg, 10.0% purity, 1.00 eq) under N₂ atmosphere. The suspension was degassed and purged with H₂ 3 times. The mixture was stirred under H₂ at 25° C. for 1 h. The reaction mixture was filtered, and the filtrate was concentrated under reduced pressure to afford (5R)-3-((2-((S)-amino((1S,3R)-3-cyclopropylcyclobutyl)methyl)imidazo[1,2-b]pyridazin-6-yl)methyl)-5-(trifluoromethyl)piperidin-2-one (38.0 mg, 90.2 μmol, 84% yield) as a yellow solid. LCMS [M+H]+=422.2 m/z.

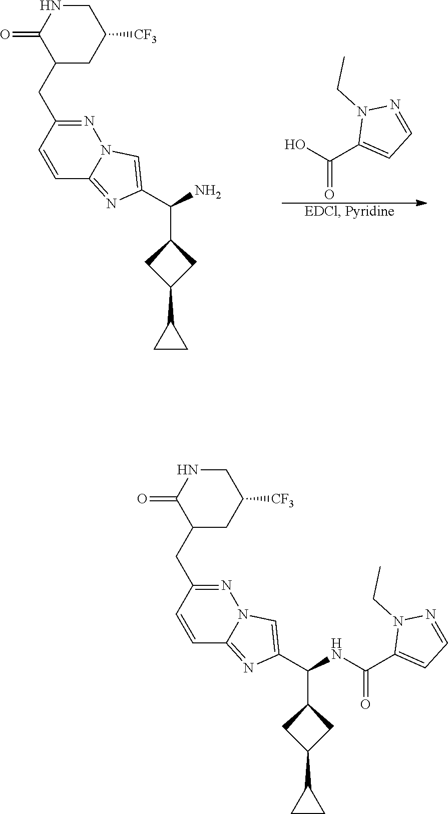

N-((1S)—((1S,3R)-3-cyclopropylcyclobutyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5R)-3-((2-((S)-amino(3-methylbicyclo[1.1.1]pentan-1-yl)methyl)imidazo[1,2-b]pyridazin-6-yl)methyl)-5-(trifluoromethyl)piperidin-2-one (35.0 mg, 83.0 μmol, 1.00 eq), 1-ethyl-1H-pyrazole-5-carboxylic acid (17.4 mg, 124 μmol, 1.50 eq) and was purified by prep-HPLC (column: DAICEL CHIRALPAK AD (250 mm×30 mm, 10 μm); mobile phase: [CO₂-EtOH (0.1% NH₃H₂O)]; B %:45%, isocratic elution mode), SFC (Column: Chiralpak AD-3 50×4.6 mm ID, 3 um Mobile phase: Phase A for CO₂, and Phase B for IPA (0.05% DEA)) to afford Compound 179, N-((1S)-((1S,3R)-3-cyclopropylcyclobutyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, (18.14 mg, 32.3 μmol, 39% yield) as a light yellow solid. LCMS [M+H]+=544.3 m/z.

### Example 180: Preparation of Compound 180

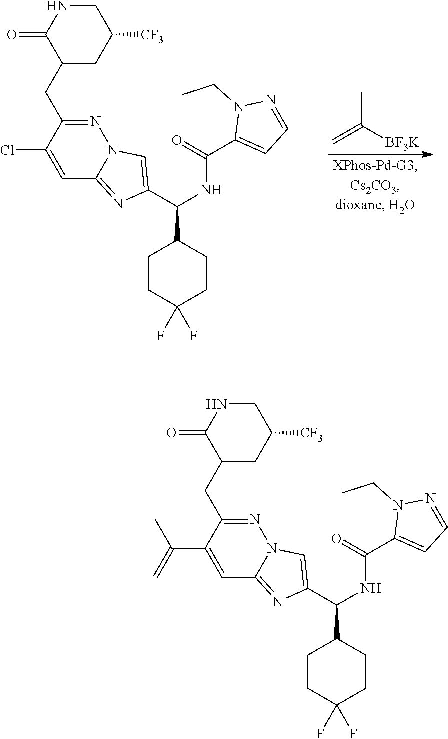

A mixture of N—((S)-(7-chloro-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (70.0 mg, 116 μmol, 1.00 eq), trifluoro(prop-1-en-2-yl)-l⁴-borane, potassium salt (430 mg, 2.91 mmol, 25.0 eq), Cs₂CO₃ (113 mg, 348 μmol, 3.00 eq), and XPhos Pd G3 (9.84 mg 11.6 μmol, 0.100 eq) in dioxane (4.00 mL) and H₂O (0.50 mL) was degassed and purged with N₂ 3 times, and then the mixture was stirred at 100° C. for 1 h under N₂ atmosphere. The reaction mixture was partitioned between EtOAc and water. The organic phase was separated, dried over anhydrous Na₂SO₄, filtered, and concentrated under reduced pressure to give a residue, which was purified by prep-TLC (SiO₂, 10:1 DCM:MeOH) to afford N-((1S)-(4,4-difluorocyclohexyl)(6-(((5R)-2-oxo-5-(trifluoromethyl)piperidin-3-yl)methyl)-7-(prop-1-en-2-yl)imidazo[1,2-b]pyridazin-2-yl)methyl)-1-ethyl-1H-pyrazole-5-carboxamide (35.0 mg, 57.6 μmol, 50% yield) as a white solid. LCMS [M+H]+=608.4 m/z.

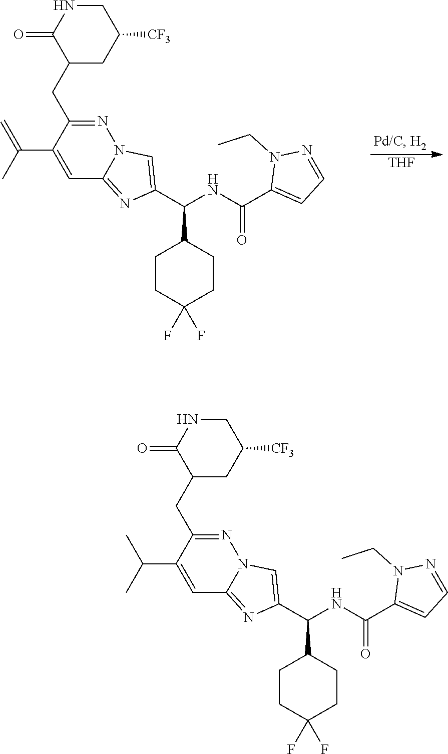 [SMILES: C=C(C)c1cc2nc([C@@H](NC(=O)c3ccnn3CC)C3CCC(F)(F)CC3)cn2nc1CC1C[C@@H](C(F)(F)F)CNC1=O ; CCn1nccc1C(=O)N[C@H](c1cn2nc(CC3C[C@@H](C(F)(F)F)CNC3=O)c(C(C)C)cc2n1)C1CCC(F)(F)CC1 ; "THF"]

To a solution of N-((1S)-(4,4-difluorocyclohexyl)(6-(((5R)-2-oxo-5-(trifluoromethyl)piperidin-3-yl)methyl)-7-(prop-1-en-2-yl)imidazo[1,2-b]pyridazin-2-yl)methyl)-1-ethyl-1H-pyrazole-5-carboxamide (35.0 mg, 57.6 μmol, 1.00 eq) in THF (3.00 mL) was added Pd/C (100 mg, 10% purity). The reaction mixture was stirred at 25° C. for 1.5 h under H₂ (15 psi). The reaction mixture was filtered, and the precipitate washed with MeOH, then concentrated under reduced pressure to afford Compound 180, N-((1S)-(4,4-difluorocyclohexyl)(7-isopropyl-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, (23.6 mg 37.7 μmol, 66% yield) as a white solid. LCMS [M+H]+=610.3 m/z.

### Example 181: Preparation of Compounds 181 and 182

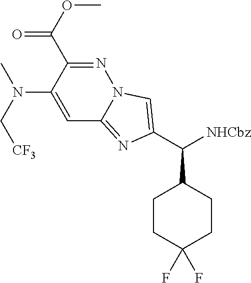 [SMILES: COC(=O)c1nn2cc([C@@H](NC(=O)OCc3ccccc3)C3CCC(F)(F)CC3)nc2cc1N(C)CC(F)(F)F]

Methyl (S)-2-((((benzyloxy)carbonyl)amino)(4,4-difluorocyclohexyl)methyl)-7-(methyl(2,2,2-trifluoroethyl)amino)imidazo[1,2-b]pyridazine-6-carboxylate was synthesized according to General Procedure 10, employing 2,2,2-trifluoro-N-methylethan-1-amine, General Procedure 11, employing methyl 6-chloro-4-(methyl(2,2,2-trifluoroethyl)amino)pyridazine-3-carboxylate, General Procedure 12, employing methyl 6-((tert-butoxycarbonyl)amino)-4-(methyl(2,2,2-trifluoroethyl)amino)pyridazine-3-carboxylate, and General Procedure 1, employing methyl 6-amino-4-(methyl(2,2,2-trifluoroethyl)amino)pyridazine-3-carboxylate and benzyl (S)-(3-bromo-1-(4,4-difluorocyclohexyl)-2-oxopropyl)carbamate.

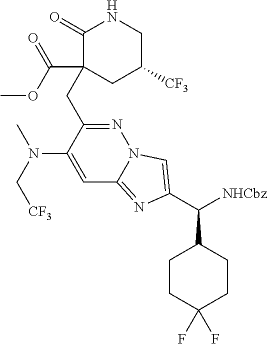 [SMILES: COC(=O)C1(Cc2nn3cc([C@@H](NC(=O)OCc4ccccc4)C4CCC(F)(F)CC4)nc3cc2N(C)CC(F)(F)F)C[C@@H](C(F)(F)F)CNC1=O]

Methyl (5R)-3-((2-((S)-(((benzyloxy)carbonyl)amino)(4,4-difluorocyclohexyl)methyl)-7-(methyl(2,2,2-trifluoroethyl)amino)imidazo[1,2-b]pyridazin-6-yl)methyl)-2-oxo-5-(trifluoromethyl)piperidine-3-carboxylate was synthesized according to General Procedure 13, employing methyl (S)-2-((((benzyloxy)carbonyl)amino)(4,4-difluorocyclohexyl)methyl)-7-(methyl(2,2,2-trifluoroethyl)amino)imidazo[1,2-b]pyridazine-6-carboxylate, General Procedure 7, employing benzyl (S)-((4,4-difluorocyclohexyl)(6-(hydroxymethyl)-7-(methyl(2,2,2-trifluoroethyl)amino)imidazo[1,2-b]pyridazin-2-yl)methyl)carbamate, and General Procedure 14, employing benzyl (S)-((6-(chloromethyl)-7-(methyl(2,2,2-trifluoroethyl)amino)imidazo[1,2-b]pyridazin-2-yl)(4,4-difluorocyclohexyl)methyl)carbamate and methyl (5R)-2-oxo-5-(trifluoromethyl)piperidine-3-carboxylate.

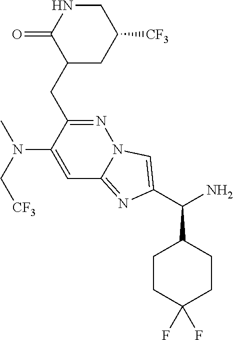 [SMILES: CN(CC(F)(F)F)c1cc2nc([C@@H](N)C3CCC(F)(F)CC3)cn2nc1CC1C[C@@H](C(F)(F)F)CNC1=O]

(5R)-3-((2-((S)-amino(4,4-difluorocyclohexyl)methyl)-7-(methyl(2,2,2-trifluoroethyl)amino)imidazo[1,2-b]pyridazin-6-yl)methyl)-5-(trifluoromethyl)piperidin-2-one was synthesized according to General Procedure 5, employing methyl (5R)-3-((2-((S)-(((benzyloxy)carbonyl)amino)(4,4-difluorocyclohexyl)methyl)-7-(methyl(2,2,2-trifluoroethyl)amino)imidazo[1,2-b]pyridazin-6-yl)methyl)-2-oxo-5-(trifluoromethyl)piperidine-3-carboxylate, General Procedure 6, employing (5R)-3-((2-((S)-(((benzyloxy)carbonyl)amino)(4,4-difluorocyclohexyl)methyl)-7-(methyl(2,2,2-trifluoroethyl)amino)imidazo[1,2-b]pyridazin-6-yl)methyl)-2-oxo-5-(trifluoromethyl)piperidine-3-carboxylic acid, and General Procedure 2, employing benzyl ((1S)-(4,4-difluorocyclohexyl)(7-(methyl(2,2,2-trifluoroethyl)amino)-6-(((5R)-2-oxo-5-(trifluoromethyl)piperidin-3-yl)methyl)imidazo[1,2-b]pyridazin-2-yl)methyl)carbamate.

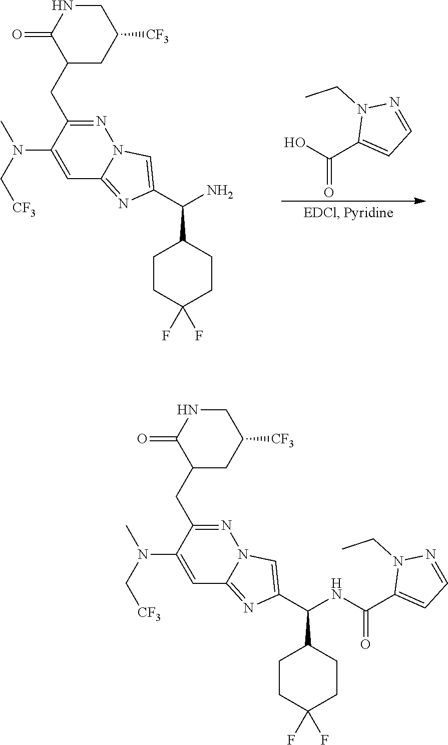

N-((1S)-(4,4-difluorocyclohexyl)(7-(methyl(2,2,2-trifluoroethyl)amino)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5R)-3-((2-((S)-amino(4,4-difluorocyclohexyl)methyl)-7-(methyl(2,2,2-trifluoroethyl)amino)imidazo[1,2-b]pyridazin-6-yl)methyl)-5-(trifluoromethyl)piperidin-2-one (90.0 mg, 161 μmol, 1.00 eq), 1-ethyl-1H-pyrazole-5-carboxylic acid (34.0 mg, 242 μmol, 1.50 eq) and was purified by prep-TLC (SiO₂, DCM:MeOH) to afford N-((1S)-(4,4-difluorocyclohexyl)(7-(methyl(2,2,2-trifluoroethyl)amino)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (60.0 mg, 88.4 μmol, 55% yield) as a yellow solid. LCMS [M+H]+=679.4 m/z.

The title compound Compound 181 was isolated as the first eluting, single stereoisomer by chiral SFC purification (condition: column: REGIS (S, S) WHELK-O1 (250 mm×25 mm, 10 μm); mobile phase: [CO₂-MeOH (0.1% NH₃H₂O)]; B %: 40%, isocratic elution mode); (23.28 mg, 31.7 μmol, 36% yield) and was obtained as a white solid. LCMS [M+H]+=679.3 m/z.

The title compound Compound 182 was isolated as the second eluting, single stereoisomer by chiral SFC purification (condition: column: REGIS (S, S) WHELK-O1 (250 mm×25 mm, 10 μm); mobile phase: [CO₂-MeOH (0.10% NH₃H₂O)]; B %: 40%, isocratic elution mode); (18.03 mg, 25.6 μmol, 29% yield) and was obtained as a white solid. LCMS [M+H]+=679.3 m/z.

### Example 182: Preparation of Compound 183

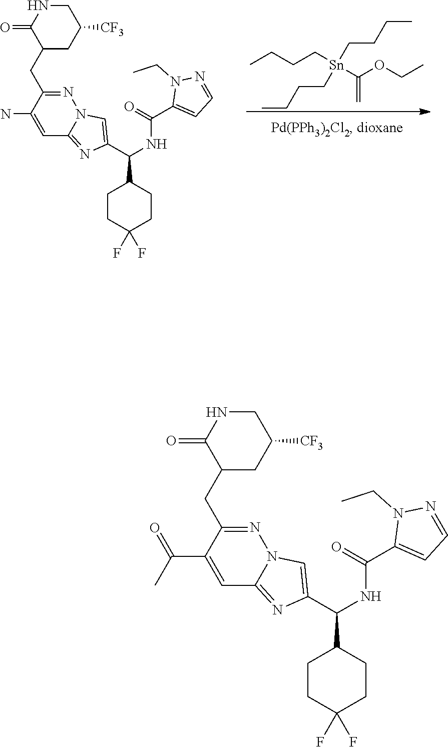 [SMILES: CCn1nccc1C(=O)N[C@H](c1cn2nc(CC3C[C@@H](C(F)(F)F)CNC3=O)c(N)cc2n1)C1CCC(F)(F)CC1 ; C=[C](OCC)[Sn]([CH2]CCC)([CH2]CCC)[CH2]CCC ; CCn1nccc1C(=O)N[C@H](c1cn2nc(CC3C[C@@H](C(F)(F)F)CNC3=O)c(C(C)=O)cc2n1)C1CCC(F)(F)CC1]

N-((1S)-(7-chloro-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (200 mg 332 μmol, 1.00 eq), tributyl(1-ethoxyvinyl)stannane (680 mg, 1.88 mmol, 636 μL, 5.67 eq) and Pd(PPh₃)₂Cl₂ (18.7 mg, 26.6 μmol, 0.0800 eq) were taken up into a microwave tube in dioxane (3.00 mL). The sealed tube was heated at 110° C. for 18 h under microwave irradiation. The reaction mixture was quenched by the addition of saturated aqueous solution of potassium fluoride at 20° C., and then diluted with 2 M HCl (20.0 mL) and stirred at 20° C. for 2 h. The reaction mixture was adjusted to pH=7 with a saturated aqueous solution of NaCO₃, filtered and extracted with ethyl acetate. The combined organic layers were washed with brine, dried over anhydrous Na₂SO₄, filtered and concentrated under reduced pressure to give a residue, which was purified by Prep-TLC (SiO₂, DCM:MeOH=10:1) to afford N-((1S)-(7-acetyl-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (100 mg, 164 μmol, 49% yield) as a yellow oil. ¹H NMR (400 MHz, CDCl₃) δ 8.26 (s, 1H), 7.89 (s, 1H), 7.46 (d, J=2.0 Hz, 1H), 7.05 (d, J=8.8 Hz, 1H), 6.60 (d, J=2.0 Hz, 1H), 6.10 (br d, J=2.0 Hz, 1H), 5.28 (t, J=8.0 Hz, 1H), 4.58 (q, J=7.2 Hz, 2H), 3.86-3.72 (m, 1H), 3.63-3.50 (m, 1H), 3.50-3.41 (m, 1H), 3.39-3.29 (m, 1H), 3.14-2.99 (m, 1H), 2.71 (s, 1H), 2.67 (s, 3H), 2.25 (d, J=13.2 Hz, 1H), 2.20-2.09 (m, 2H), 2.02-1.80 (m, 3H), 1.57-1.49 (m, 1H), 1.43 (t, J=7.2 Hz, 3H), 1.40-1.34 (m, 2H), 1.34-1.28 (br d, J=8.4 Hz, 2H).

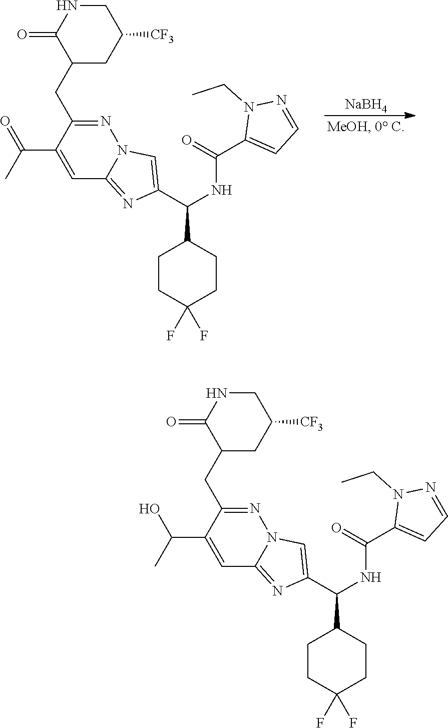 [SMILES: CCn1nccc1C(=O)N[C@H](c1cn2nc(CC3C[C@@H](C(F)(F)F)CNC3=O)c(C(C)=O)cc2n1)C1CCC(F)(F)CC1 ; "NaBH4" ; CCn1nccc1C(=O)N[C@H](c1cn2nc(CC3C[C@@H](C(F)(F)F)CNC3=O)c(C(C)O)cc2n1)C1CCC(F)(F)CC1]

To a solution of N-((1S)-(7-acetyl-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (100 mg, 164 μmol, 1.00 eq) in MeOH (2.00 mL) was added NaBH₄ (50.0 mg, 1.32 mmol, 8.06 eq) at 0° C. The reaction mixture was stirred at 25° C. for 1 h. The reaction mixture was quenched by the addition of a saturated aqueous solution of ammonium chloride at 25° C., and then extracted with ethyl acetate. The combined organic layers were dried over anhydrous Na₂SO₄, filtered and concentrated under reduced pressure to give a residue, which was purified by Prep-TLC (SiO₂, DCM:MeOH=10:1) to afford N-((1S)-(4,4-difluorocyclohexyl)(7-(1-hydroxyethyl)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (60.0 mg, 98.1 μmol, 60% yield) as a yellow oil. ¹H NMR (400 MHz, MeOD) δ 8.07-7.95 (m, 2H), 7.48 (d, J=2.0 Hz, 1H), 6.85 (d, J=2.0 Hz, 1H), 5.24-5.16 (m, 1H), 5.08 (m, 6.1 Hz, 1H), 4.51 (q, J=7.2 Hz, 2H), 3.61-3.41 (m, 3H), 3.28-3.09 (m, 2H), 3.01-2.85 (m, 1H), 2.38-2.14 (m, 2H), 2.13-2.05 (m, 1H), 2.00-1.56 (m, 6H), 1.51 (d, J=6.8 Hz, 3H), 1.49-1.37 (m, 2H), 1.37-1.33 (m, 3H).

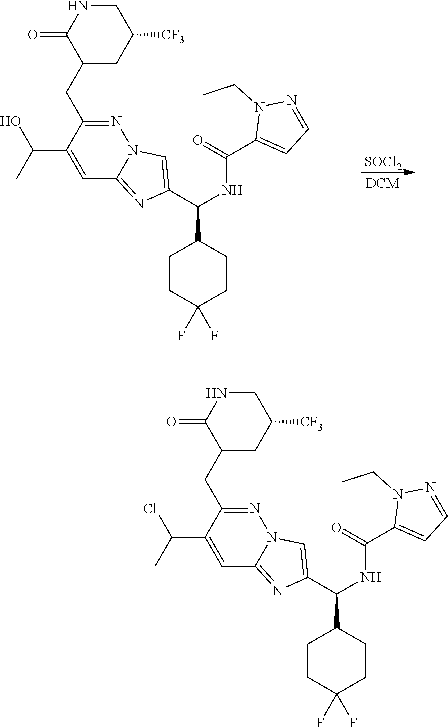

To a solution of N-((1S)-(4,4-difluorocyclohexyl)(7-(1-hydroxyethyl)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (50.0 mg, 81.7 μmol, 1.00 eq) in DCM (3.00 mL) was added SOCl₂ (48.6 mg, 409 μmol, 29.7 μL, 5.00 eq) at 0° C. The reaction mixture was stirred at 25° C. for 12 h. The reaction mixture was concentrated under reduced pressure to afford N-((1S)-(7-(1-chloroethyl)-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (60.0 mg, crude, HCl salt) as a yellow oil. LCMS [M+H]⁺=630.4 m/z.

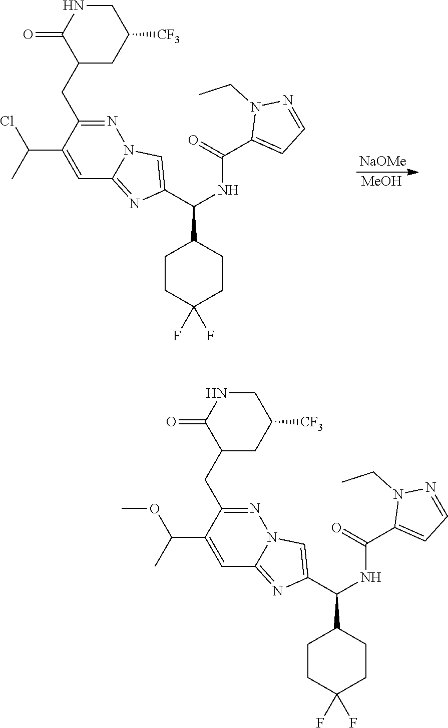 [SMILES: "MeOH" ; CCn1nccc1C(=O)N[C@H](c1cn2nc(CC3C[C@@H](C(F)(F)F)CNC3=O)c(C(C)Cl)cc2n1)C1CCC(F)(F)CC1 ; CCn1nccc1C(=O)N[C@H](c1cn2nc(CC3C[C@@H](C(F)(F)F)CNC3=O)c(C(C)OC)cc2n1)C1CCC(F)(F)CC1 ; "NaOMe"]

To a solution of N-((1S)-(7-(1-chloroethyl)-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (60.0 mg, 90.0 μmol, 1.00 eq, HCl salt) in MeOH (2.00 mL) was added sodium methoxide (5.4 M, 166 μL, 10.0 eq) at 0° C. The reaction mixture was stirred at 30° C. for 16 h. The reaction mixture was adjusted to pH=7 with a saturated aqueous solution of ammonium chloride at 0° C., and then diluted with water and extracted with ethyl acetate. The combined organic layers were dried over anhydrous Na₂SO₄, filtered and concentrated under reduced pressure to give a residue, which was purified by Prep-HPLC (FA condition, column: Phenomenex luna C 18 150×25 mm, 10 μm; mobile phase: [water (FA)-ACN]; gradient: 38%-58% B) and Prep-SFC (column: REGIS (S, S) WHELK-O1 (250 mm×25 mm, 10 μm); mobile phase: [CO₂-EtOH (0.1% NH₃H₂O)]; B %: 45%) to afford Compound 183, N-((1S)-(4,4-difluorocyclohexyl)(7-(1-methoxyethyl)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, (20.4 mg 3 1.9 μmol, 40% yield) as a white solid. LCMS [M+H]⁺=626.4 m/z.

### Example 183: Preparation of Compound 184

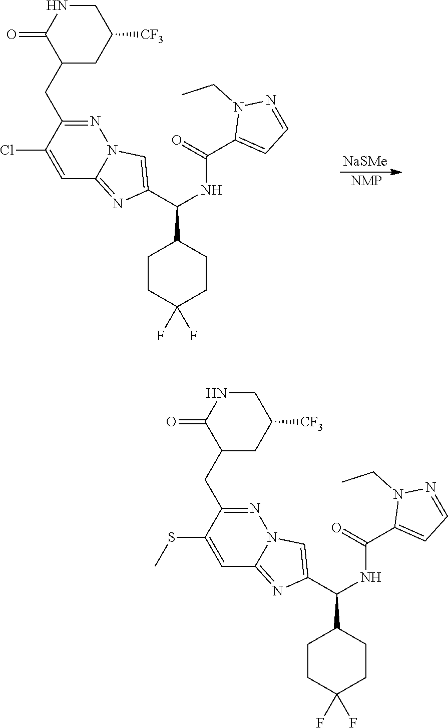

To a solution of N—((S)-(7-chloro-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (0.200 g, 332 μmol, 1.00 eq) in NMP (2.00 mL) was added sodium thiomethoxide (50.0 mg, 713 μmol, 45.4 μL, 2.15 eq). The reaction mixture was stirred at 25° C. for 1 h. The reaction mixture was diluted with water and extracted with DCM. The combined organic layers were washed with brine, dried over anhydrous Na₂SO₄, filtered and concentrated under reduced pressure to give a residue, which was purified by Prep-TLC (SiO₂, DCM:MeOH=10:1) to afford N-((1S)-(4,4-difluorocyclohexyl)(7-(methylthio)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (0.100 g, crude) as a white solid. LCMS [M+H]+=614.3 m/z.

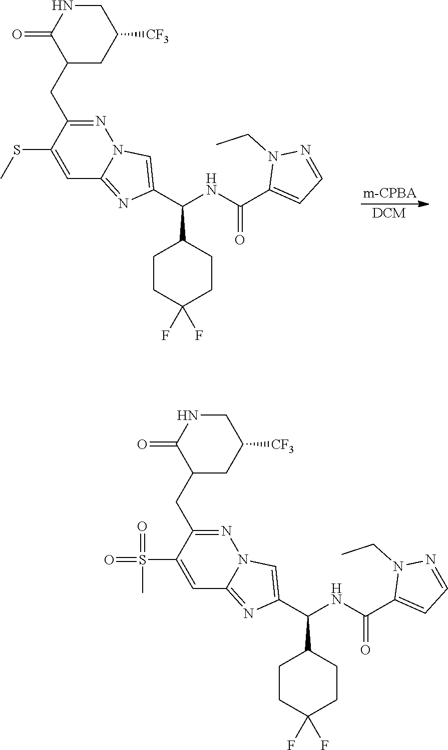

To a solution of N-((1S)-(4,4-difluorocyclohexyl)(7-(methylthio)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (50.0 mg, 81.4 μmol, 1.00 eq) in DCM (2.00 mL) was added m-CPBA (43.9 mg, 203 μmol, 80% purity, 2.50 eq) at 0° C. The reaction mixture was stirred at 25° C. for 1 h. The reaction mixture was diluted with water and extracted with DCM. The combined organic layers were washed with brine, dried over anhydrous Na₂SO₄, filtered and concentrated under reduced pressure to give a residue, which was purified by Prep-HPLC (column: Waters Xbridge 150×25 mm, 10 μm; mobile phase: [water(NH₄HCO₃)-ACN]; gradient:33%-63% B) and Prep SFC (column: REGIS(S, S) WHELK-O1 (250 mm×25 mm, 10 μm); mobile phase: [CO₂-MeOH (0.1% NH₃H₂O)]; B %: 50%) to afford Compound 184, N-((1S)-(4,4-difluorocyclohexyl)(7-(methylsulfonyl)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, (6.26 mg, 9.22 μmol, 11% yield) as a yellow solid. LCMS [M+H]+=646.3 m/z.

### Example 184: Preparation of Compound 185

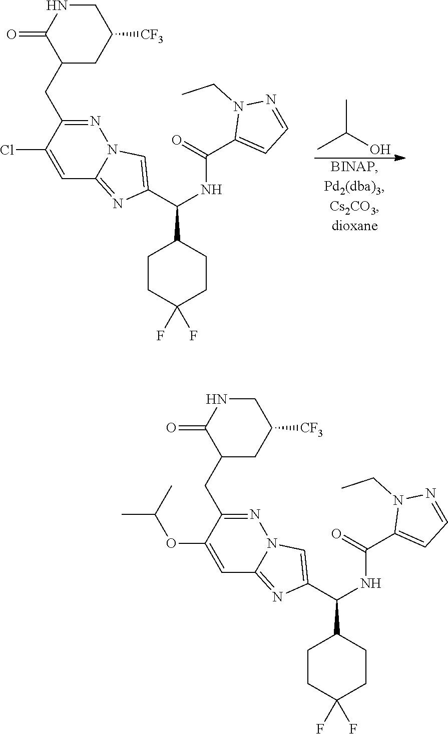

A mixture of N—((S)-(7-chloro-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (80.0 mg, 133 μmol, 1.00 eq), propan-2-ol (160 mg, 2.66 mmol, 203 μL, 20.0 eq), BINAP (24.8 mg, 39.9 μmol, 0.30 eq), Cs₂CO₃ (130 mg, 399 μmol, 3.00 eq) and Pd₂(dba)₃ (12.2 mg, 13.3 μmol, 0.10 eq) in dioxane (1.00 mL) was degassed and purged with N₂ 3 times, and then the mixture was stirred at 110° C. for 24 h under N₂ atmosphere. The reaction mixture was partitioned between EtOAc and water. The organic phase was separated, dried over anhydrous Na₂SO₄, filtered and concentrated under reduced pressure to give a residue, which was purified by Prep-HPLC (column: Phenomenex Luna C18 150×25 mm, 10 μm; mobile phase: [water (FA)-ACN]; gradient: 31%-61% B) to afford Compound 185, N-((1S)-(4,4-difluorocyclohexyl)(7-isopropoxy-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, (7.67 mg, 12.18 μmol, 9% yield) as a brown solid. LCMS [M+H]+=626.4 m/z.

### Example 185: Preparation of Compound 186

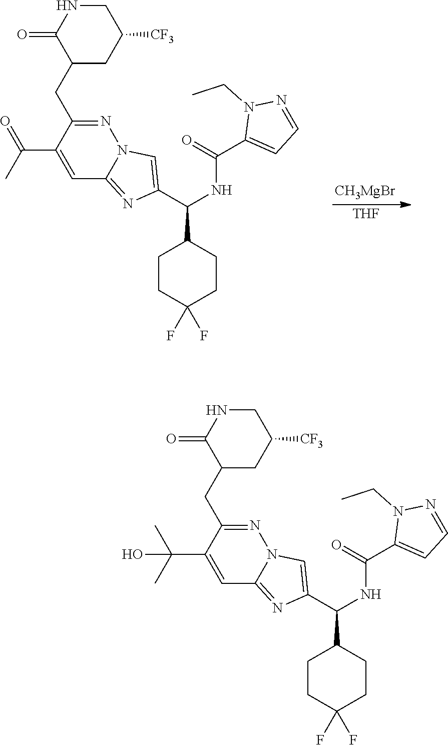

To a solution of N-((1S)-(7-acetyl-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (50.0 mg, 82.0 μmol, 1.00 eq) in THF (3.00 mL) was added methyl magnesium bromide (3 M, 273 μL, 10.0 eq) at 0° C. The reaction mixture was stirred at 25° C. for 1 h. The reaction mixture was quenched by the addition of a saturated aqueous solution of ammonium chloride at 0° C., and then diluted with water and extracted with ethyl acetate. The combined organic layers were dried over anhydrous Na₂SO₄, filtered and concentrated under reduced pressure to give a residue, which was purified by Prep-HPLC (FA condition, column: Phenomenex luna C 18 150×25 mm, 10 μm; mobile phase: [water (FA)-ACN]; gradient: 32%-52% B) to afford Compound 186, N-((1S)-(4,4-difluorocyclohexyl)(7-(2-hydroxypropan-2-yl)-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, (12.4 mg, 19.2 μmol, 27% yield) as a yellow solid. LCMS [M+H]+=626.2 m/z.

### Example 186: Preparation of Compound 187

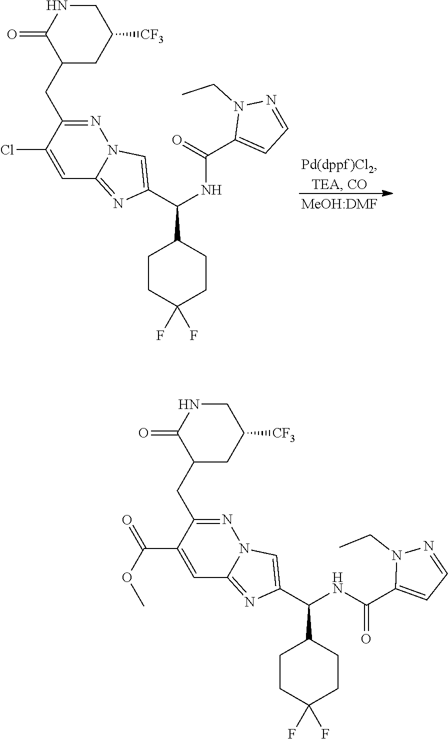 [SMILES: CCn1nccc1C(=O)N[C@H](c1cn2nc(CC3C[C@@H](C(F)(F)F)CNC3=O)c(Cl)cc2n1)C1CCC(F)(F)CC1 ; CCn1nccc1C(=O)N[C@H](c1cn2nc(CC3C[C@@H](C(F)(F)F)CNC3=O)c(C(=O)OC)cc2n1)C1CCC(F)(F)CC1]

To a solution of N—((S)-(7-chloro-6-(((5R)-2-oxo-5-(trifluoromethyl)piperidin-3-yl)methyl)imidazo[1,2-b]pyridazin-2-yl)(4,4-difluorocyclohexyl)methyl)-1-ethyl-1H-pyrazole-5-carboxamide (70.0 mg, 116 μmol, 1.00 eq) in MeOH (2.00 mL) and DMF (0.500 mL) was added Pd(dppf)Cl₂ (42.5 mg, 58.1 μmol, 0.500 eq) and TEA (35.3 mg, 349 μmol, 48.6 μL, 3.00 eq) under N₂ atmosphere. The suspension was degassed and purged with carbon monoxide 3 times. The reaction mixture was stirred under carbon monoxide (1 MPa) in autoclave at 110° C. for 12 h. The reaction mixture was filtered and the filtrate was diluted with H₂O and extracted with ethyl acetate. The combined organic layers were washed with brine and the organic layers were dried over anhydrous Na₂SO₄, filtered and concentrated under reduced pressure to afford methyl 2-((S)-(4,4-difluorocyclohexyl)(1-ethyl-1H-pyrazole-5-carboxamido)methyl)-6-(((5R)-2-oxo-5-(trifluoromethyl)piperidin-3-yl)methyl)imidazo[1,2-b]pyridazine-7-carboxylate (60.0 mg, 95.9 mol, 82% yield) as a yellow oil. LCMS [M+H]⁺=626.4 m/z.

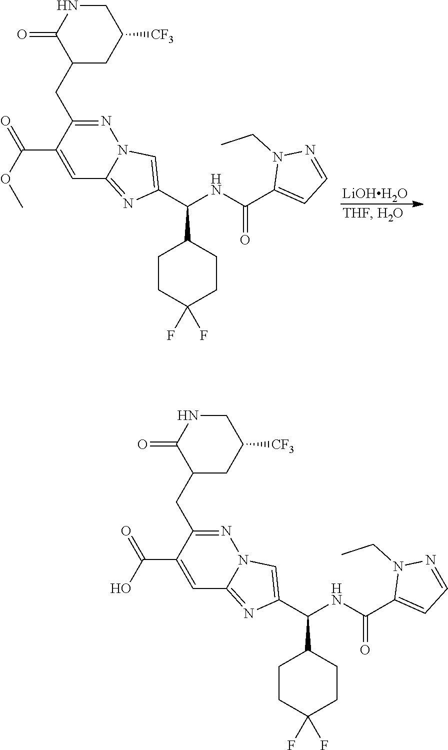

To a solution of N-((1S)-(4,4-difluorocyclohexyl)(7-isopropyl-6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (60.0 mg, 95.9 μmol, 1.00 eq) in THF (1.00 mL) and H₂O (0.200 mL) was added LiOH·H₂O (16.1 mg, 384 μmol, 4.00 eq). The reaction mixture was stirred at 25° C. for 2 h. The reaction mixture was adjusted to pH=6 with 1 M HCl, then concentrated under reduced pressure to give a residue, which was purified by Prep-HPLC (column: Phenomenex luna C18 150×25 mm, 10 μm; mobile phase: [water (FA)-ACN]; gradient: 32%-62% B) to afford Compound 187, 2-((S)-(4,4-difluorocyclohexyl)(1-ethyl-1H-pyrazole-5-carboxamido)methyl)-6-(((5R)-2-oxo-5-(trifluoromethyl)piperidin-3-yl)methyl)imidazo[1,2-b]pyridazine-7-carboxylic acid, (31.42 mg, 47.9 μmol, 50% yield) as a gray solid. LCMS [M+H]+=612.3 m/z.

### Example 187: Preparation of Compound 188

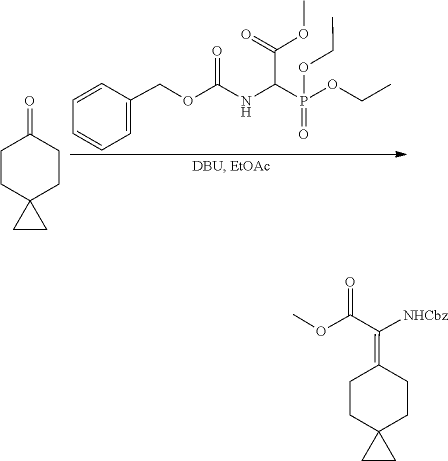

To a solution of methyl 2-(((benzyloxy)carbonyl)amino)-2-(diethoxyphosphoryl)acetate (4.20 g, 33.8 mmol, 1.00 eq) in EtOAc (40.0 mL) was added DBU (6.69 g, 43.9 mmol, 6.63 mL, 1.30 eq) and stirred at 25° C. for 0.5 h. Then a solution of spiro[2.5]octan-6-one (12.7 g, 35.5 mmol, 1.05 eq) in EtOAc (20.0 mL) was added and stirred at 25° C. for 15 h. The reaction mixture was concentrated under reduced pressure to give a residue, which was purified by column chromatography (SiO₂, PE/EtOAc=30/1 to 3/1) to afford methyl 2-(((benzyloxy)carbonyl)amino)-2-(spiro[2.5]octan-6-ylidene)acetate (5.00 g, 15.1 mmol, 45% yield) as a white solid. ¹H NMR (400 MHz, CDCl₃) δ 7.45-7.30 (m, 5H), 6.02-5.77 (m, 1H), 5.15 (s, 2H), 3.75-3.53 (m, 3H), 2.80-2.77 (m, 2H), 2.40-2.37 (m, 2H), 1.47-1.44 (m, 4H), 0.37-0.29 (m, 4H).

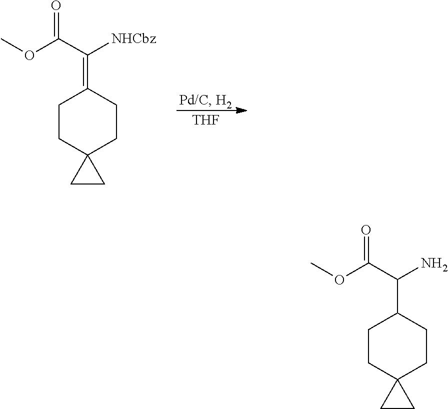 [SMILES: COC(=O)C(NC(=O)OCc1ccccc1)=C1CCC2(CC1)CC2 ; COC(=O)C(N)C1CCC2(CC1)CC2]

To a solution of methyl 2-(((benzyloxy)carbonyl)amino)-2-(spiro[2.5]octan-6-ylidene)acetate (1.70 g, 5.16 mmol, 1.00 eq) in THE (20.0 mL) was added Pd/C (0.300 g, 10% purity) under N₂. The suspension was degassed under vacuum and purged with H₂ 3 times. The reaction mixture was stirred under H₂ (15 psi) at 25° C. for 6 h. The reaction mixture was filtered with MeOH and the filter was concentrated under reduced pressure to give a residue, which was purified by column chromatography (SiO₂, PE/EtOAc=30/1 to 0/1) to afford methyl 2-amino-2-(spiro[2.5]octan-6-yl)acetate (0.720 g, 3.65 mmol, 710% yield) as a yellow oil. ¹H NMR (400 MHz, CDCl₃) δ 3.72 (s, 3H), 3.33 (d, J=5.2 Hz, 1H), 1.75-1.57 (m, 5H), 1.41-1.28 (m, 2H), 0.93-0.86 (m, 2H), 0.28-0.14 (m, 4H).

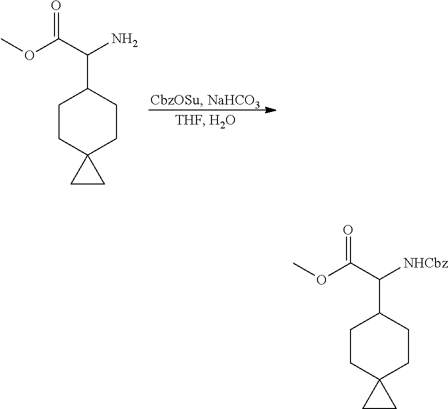 [SMILES: COC(=O)C(N)C1CCC2(CC1)CC2 ; COC(=O)C(NC(=O)OCc1ccccc1)C1CCC2(CC1)CC2]

To a solution of methyl 2-amino-2-(spiro[2.5]octan-6-yl)acetate (0.720 g, 3.65 mmol, 1.00 eq) and CbzOSu (1.36 g, 5.47 mmol, 1.50 eq) in THF (20.0 mL) was added a solution of NaCO₃ (613 mg, 7.30 mmol, 284 μL, 2.00 eq) in water (5.00 mL) at 0° C. The reaction mixture was stirred at 25° C. for 12 h. The reaction mixture was diluted with water and extracted with ethyl acetate. The combined organic layers were washed with water followed by brine, dried over anhydrous Na₂SO₄, filtered and concentrated under reduced pressure to give a residue, which was purified by column chromatography (SiO₂, PE/EtOAc=30/1 to 5/1) to afford methyl 2-(((benzyloxy)carbonyl)amino)-2-(spiro[2.5]octan-6-yl)acetate (1.00 g, 3.02 mmol, 83% yield) as a colorless oil. ¹H NMR (400 MHz, CDCl₃) δ 7.38-7.32 (m, 5H), 5.30-5.28 (m, 1H), 5.13 (s, 2H), 4.38-4.34 (m, 1H), 3.76 (s, 3H), 1.86-1.64 (m, 4H), 1.56-1.54 (m, 1H), 1.35-1.20 (m, 2H), 0.91-0.87 (m, 2H), 0.30-0.26 (m, 2H), 0.19-0.15 (m, 2H).

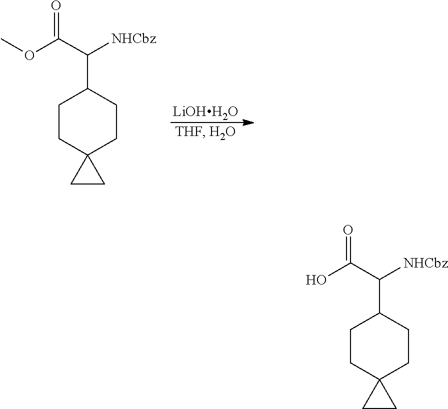 [SMILES: COC(=O)C(NC(=O)OCc1ccccc1)C1CCC2(CC1)CC2 ; O=C(NC(C(=O)O)C1CCC2(CC1)CC2)OCc1ccccc1]

To a solution of methyl 2-(((benzyloxy)carbonyl)amino)-2-(spiro[2.5]octan-6-yl)acetate (0.500 g, 1.51 mmol, 1.00 eq) in THF (6.00 mL) was added a solution of LiOH·H₂O (126 mg, 3.02 mmol, 2.00 eq) in H₂O (2.00 mL) at 0° C. The reaction mixture was stirred at 25° C. for 4 h. The reaction mixture was diluted with water, then adjusted with 1M HCl to pH=3 at 0° C., then extracted with ethyl acetate. The combined organic layers were dried over anhydrous Na₂SO₄, filtered and concentrated under reduced pressure to afford 2-(((benzyloxy)carbonyl)amino)-2-(spiro[2.5]octan-6-yl)acetic acid (0.410 g, 1.29 mmol, 86% yield) as a white solid. ¹H NMR (400 MHz, CDCl₃) δ 9.54 (s, 1H), 7.38-7.33 (m, 5H), 5.31 (d, J=8.8 Hz, 1H), 5.13 (s, 2H), 4.42-4.38 (m, 1H), 1.93-1.71 (m, 5H), 1.39-1.26 (m, 2H), 0.91-0.88 (m, 2H), 0.30-0.17 (m, 4H).

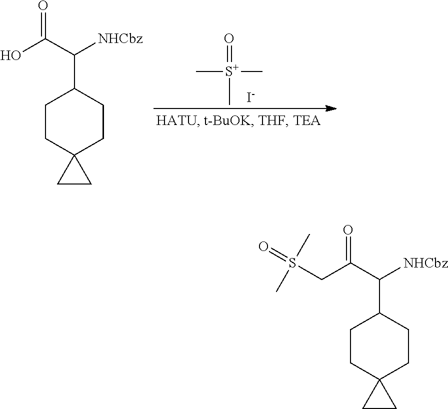

The preparation of mixture A: trimethylsulfoxonium iodide (738 mg, 3.3 6 mmol, 3.00 eq) was added to a solution of t-BuOK (376 mg, 3.36 mmol, 3.00 eq) in THF (8.00 mL) and the resulting mixture was stirred at 65° C. for 2 h under N₂. The reaction mixture was cooled to 0° C. to obtain the mixture A.

The preparation of mixture B: To a solution of 2-(((benzyloxy)carbonyl)amino)-2-(spiro[2.5]octan-6-yl)acetic acid (355 mg, 1.12 mmol, 1.00 eq) in THF (8.00 mL) was added HATU (553 mg, 1.45 mmol, 1.30 eq) and TEA (147 mg, 1.45 mmol, 202 μL, 1.30 eq) at 25° C. The reaction mixture was stirred at 25° C. for 2 h. The suspension was then filtered and the filtrate was cooled to 0° C. to obtain the mixture B.

The mixture B was added to the mixture A at 0° C. The resulting mixture was stirred at 25° C. for 1.5 h. The reaction mixture was diluted with water and extracted with ethyl acetate. The combined organic layers were dried over anhydrous Na₂SO₄, filtered and concentrated under reduced pressure to give a residue, which was purified by column chromatography (SiO₂, PE/EtOAc=73/27 to 77/23) to afford benzyl (3-(dimethyl(oxo)-l⁶-sulfaneylidene)-2-oxo-1-(spiro[2.5]octan-6-yl)propyl)carbamate (0.200 g, 510 μmol, 46% yield) as a yellow solid. ¹H NMR (400 MHz, CDCl₃) δ 7.39-7.28 (m, 5H), 5.65 (d, J=9.2 Hz, 1H), 5.12-5.11 (m, 2H), 4.75-4.35 (m, 1H), 2.06-4.03 (m, 1H), 3.43-3.32 (m, 6H), 1.76-1.64 (m, 5H), 1.30-1.18 (m, 2H), 0.90-0.88 (m, 2H), 0.27-0.14 (m, 4H).

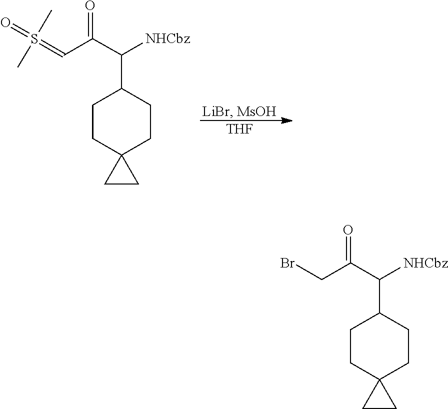 [SMILES: CS(C)(=O)=CC(=O)C(NC(=O)OCc1ccccc1)C1CCC2(CC1)CC2 ; O=C(NC(C(=O)CBr)C1CCC2(CC1)CC2)OCc1ccccc1]

The mixture B was added to the mixture A at 0° C. The resulting mixture was stirred at 25° C. for 1.5 h. The reaction mixture was diluted with water and extracted with ethyl acetate. The combined organic layers were dried over anhydrous Na₂SO₄, filtered and concentrated under reduced pressure to give a residue, which was purified by column chromatography (SiO₂, PE/EtOAc=73/27 to 77/23) to afford benzyl (3-bromo-2-oxo-1-(spiro[2.5]octan-6-yl)propyl)carbamate (0.200 g, 510 μmol, 46% yield) as a yellow solid.

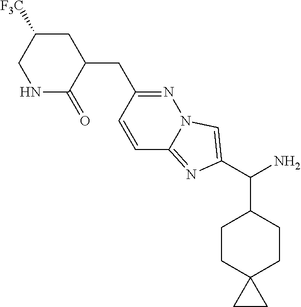 [SMILES: NC(c1cn2nc(CC3C[C@@H](C(F)(F)F)CNC3=O)ccc2n1)C1CCC2(CC1)CC2]

(5R)-3-((2-(amino(spiro[2.5]octan-6-yl)methyl)imidazo[1,2-b]pyridazin-6-yl)methyl)-5-(trifluoromethyl)piperidin-2-one was synthesized according to General Procedure 1, employing (5R)-3-((6-aminopyridazin-3-yl)methyl)-5-(trifluoromethyl)piperidin-2-one and benzyl (3-bromo-2-oxo-1-(spiro[2.5]octan-6-yl)propyl)carbamate, and General Procedure 3, employing benzyl ((6-(((5R)-2-oxo-5-(trifluoromethyl)piperidin-3-yl)methyl)imidazo[1,2-b]pyridazin-2-yl)(spiro[2.5]octan-6-yl)methyl)carbamate.

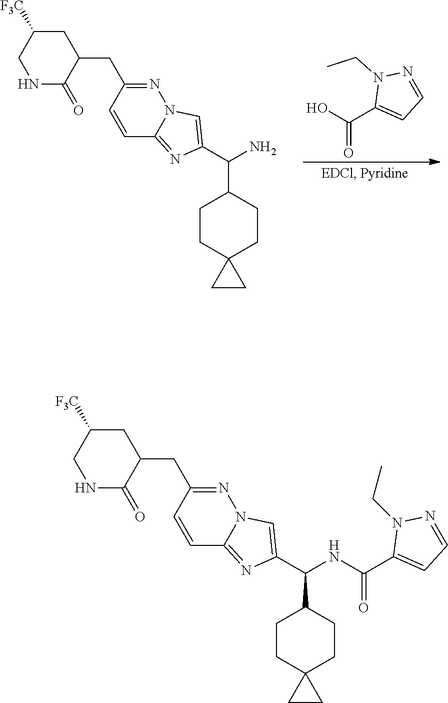

1-Ethyl-N-((1S)-(6-(((5R)-2-oxo-5-(trifluoromethyl)piperidin-3-yl)methyl)imidazo[1,2-b]pyridazin-2-yl)(spiro[2.5]octan-6-yl)methyl)-1H-pyrazole-5-carboxamide was synthesized following the General Procedure 4 with (5R)-3-((2-(amino(spiro[2.5]octan-6-yl)methyl)imidazo[1,2-b]pyridazin-6-yl)methyl)-5-(trifluoromethyl)piperidin-2-one (65.0 mg, 149 μmol, 1.00 eq), 1-ethyl-1H-pyrazole-5-carboxylic acid (27.2 mg, 194 μmol, 1.30 eq) and was purified by prep-TLC (SiO₂, DCM:MeOH) to afford 1-ethyl-N-((6-(((5R)-2-oxo-5-(trifluoromethyl)piperidin-3-yl)methyl)imidazo[1,2-b]pyridazin-2-yl)(spiro[2.5]octan-6-yl)methyl)-1H-pyrazole-5-carboxamide (45.0 mg, 80.7 μmol, 54% yield) as a white solid. LCMS [M+H]+=558.3 m/z.

The title compound Compound 188 was isolated as the first eluting, single stereoisomer by chiral SFC purification (column: DAICEL CHIRALPAK AD (250 mm×30 mm, 10 μm); mobile phase: [0.1% NH₃H₂O ETOH]; B %: 40%-40%, 3.3 min); (18.3 mg, 32.0 μmol, 40% yield) and was obtained as a white solid. LCMS [M+H]+=558.3 m/z.

### Example 188: Preparation of Compounds 189 and 190

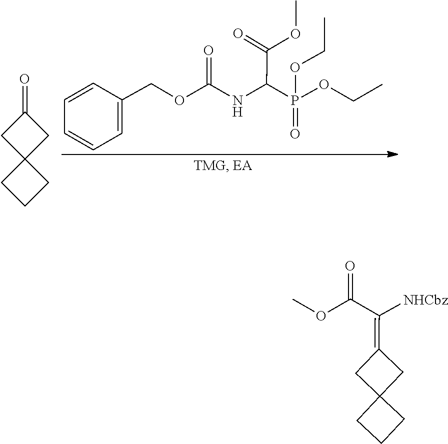

To a solution of methyl 2-(((benzyloxy)carbonyl)amino)-2-(diethoxyphosphoryl)acetate (5.00 g, 45.4 mmol, 1.00 eq) in EtOAc (50.0 mL) was added TMG (6.80 g, 59.0 mmol, 7.42 mL, 1.30 eq) and stirred at 25° C. for 0.5 h. Then a solution of spiro[3.3]heptan-2-one (16.3 g, 45.4 mmol, 1.00 eq) in EtOAc (25.0 mL) was added and stirred at 25° C. for 2 h. The reaction mixture was concentrated under reduced pressure to give a residue, which was purified by column chromatography (SiO₂, PE:EtOAc=5:1) to afford methyl 2-(((benzyloxy)carbonyl)amino)-2-(spiro[3.3]heptan-2-ylidene)acetate (9.50 g, 30.1 mmol, 66% yield) as a colorless oil. ¹H NMR (400 MHz, CDCl₃) δ 7.38-7.27 (m, 5H), 6.13 (s, 1H), 5.13 (s, 2H), 3.73 (s, 3H), 3.11 (s, 2H), 2.84 (s, 2H), 2.06-2.03 (m, 4H), 1.87-1.61 (m, 2H).

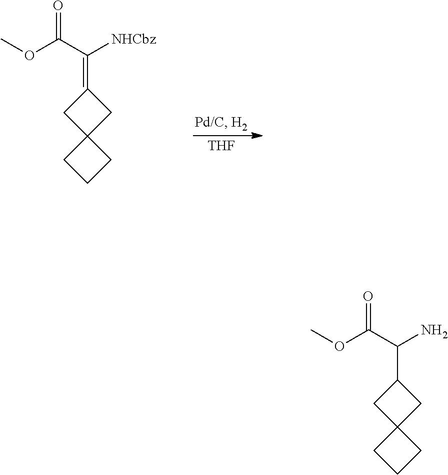 [SMILES: COC(=O)C(NC(=O)OCc1ccccc1)=C1CC2(CCC2)C1 ; COC(=O)C(N)C1CC2(CCC2)C1 ; "THF"]

To a solution of methyl 2-(((benzyloxy)carbonyl)amino)-2-(spiro[3.3]heptan-2-ylidene)acetate (2.00 g, 6.34 mmol, 1.00 eq) in THF (10.0 mL) was added Pd/C (0.200 g, 10.0% purity). The reaction mixture was degassed and purged with H₂ 3 times. The reaction mixture was stirred at 25° C. for 2 h under H₂ (15 psi) atmosphere. The reaction mixture was filtered and concentrated under reduced pressure to afford methyl 2-amino-2-(spiro[3.3]heptan-2-yl)acetate (1.16 g, 6.33 mmol, quantitative) as a colorless oil. ¹H NMR (400 MHz, DMSO) δ 3.59-3.58 (m, 3H), 3.15-3.13 (m, 1H), 2.50-2.49 (m, 1H), 2.16-1.73 (m, 10H).

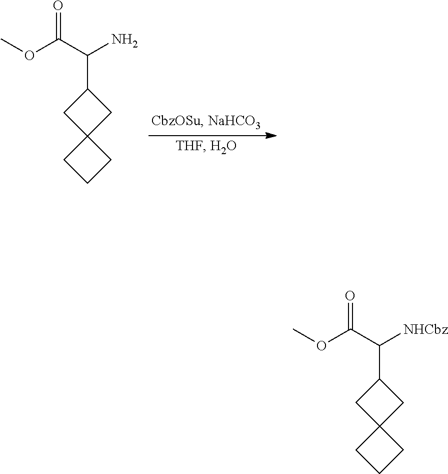

To a solution of methyl 2-amino-2-(spiro[3.3]heptan-2-yl)acetate (2.50 g, 13.6 mmol, 1.00 eq) in THF (40.0 mL) and H₂O (8.00 mL) was added sodium carbonate (2.89 g, 27.3 mmol, 2.00 eq) and CbzOSu (6.80 g, 27.3 mmol, 2.00 eq). The mixture was stirred at 25° C. for 2 h. The reaction mixture was quenched by the addition of water and extracted with ethyl acetate. The combined organic layers were dried over anhydrous Na₂SO₄, filtered and concentrated under reduced pressure to give a residue, which was purified by column chromatography (SiO₂, PE:EtOAc=15:1 to 10:1) to afford methyl 2-(((benzyloxy)carbonyl)amino)-2-(spiro[3.3]heptan-2-yl)acetate (3.00 g, 9.45 mmol, 69% yield) as a colorless oil. ¹H NMR (400 MHz, CDCl₃) δ 7.38-7.27 (m, 6H), 5.29-5.08 (m, 3H), 4.27 (t, J=8.4 Hz, 1H), 3.76-3.72 (m, 3H), 2.48-2.44 (m, 1H), 2.05-1.24 (m, 10H).

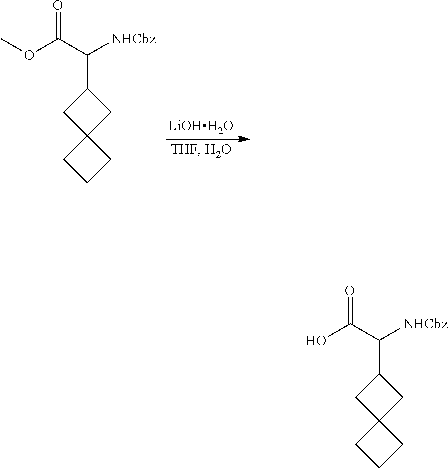

To a solution of methyl 2-(((benzyloxy)carbonyl)amino)-2-(spiro[3.3]heptan-2-yl)acetate (2.80 g, 8.82 mmol, 1.00 eq) in THE (20.0 mL) and H₂O (4.00 mL) was added LiOH·H₂O (740 mg, 17.6 mmol, 2.00 eq). The reaction mixture was stirred at 25° C. for 1 h. The reaction mixture was adjusted pH=4 with HCl (1.00 M) at 0° C., and then diluted with water and extracted with ethyl acetate. The combined organic layers were dried over anhydrous Na₂SO₄, filtered and concentrated under reduced pressure to afford 2-(((benzyloxy)carbonyl)amino)-2-(spiro[3.3]heptan-2-yl)acetic acid (2.10 g, 6.92 mmol, 79% yield) as a colorless oil.

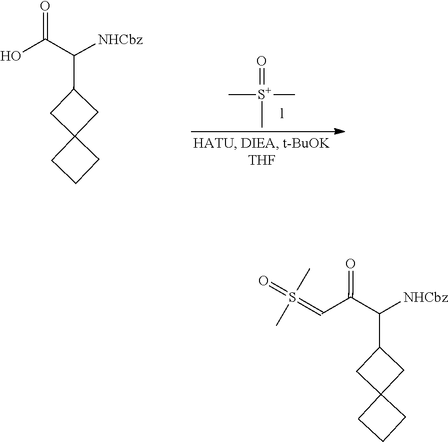

The preparation of mixture A: trimethylsulfoxonium iodide (1.84 g, 19.8 mmol, 3.00 eq) was added to a solution of t-BuOK (2.22 g, 19.8 mmol, 3.00 eq) in THF (20.0 mL) and the resulting mixture was stirred at 65° C. for 2 h under N₂. After that time, the reaction mixture was cooled to 0° C. to obtain the mixture A.

The preparation of mixture B: To a solution of 2-(((benzyloxy)carbonyl)amino)-2-(spiro[3.3]heptan-2-yl)acetic acid (2.00 g, 6.59 mmol, 1.00 eq) in THF (20.0 mL) was added HATU (3.26 g, 8.57 mmol, 1.30 eq) and TEA (867 mg, 8.57 mmol, 1.19 mL, 1.30 eq) at 0° C. The reaction mixture was stirred at 0° C. for 2 h. The suspension was then filtered and the filtrate was cooled to 0° C. to obtain the mixture B.

The mixture B was added to the mixture A at 0° C. The resulting mixture was stirred at 0° C. for 4 h. The reaction mixture was filtered and concentrated under reduced pressure to give a residue, which was purified by column chromatography (SiO₂, PE:EtOAc=1:1 to 0:1) to afford benzyl (3-(dimethyl(oxo)-l⁶-sulfaneylidene)-2-oxo-1-(spiro[3.3]heptan-2-yl)propyl)carbamate (1.50 g, 3.97 mmol, 60% yield) as a yellow oil. ¹H NMR (400 MHz, CDCl₃) δ 7.36-7.27 (m, 5H), 5.54 (d, J=8.4 Hz, 1H), 5.18-5.06 (m, 2H), 4.46-4.43 (m, 1H), 4.04 (t, J=8.4 Hz, 1H), 3.38-3.27 (m, 6H), 2.38-2.36 (m, 1H), 2.05-1.96 (m, 4H), 1.85-1.75 (m, 6H).

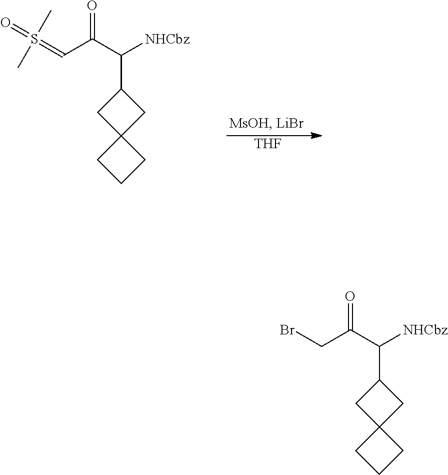 [SMILES: CS(C)(=O)=CC(=O)C(NC(=O)OCc1ccccc1)C1CC2(CCC2)C1 ; O=C(NC(C(=O)CBr)C1CC2(CCC2)C1)OCc1ccccc1 ; "THF"]

To a solution of benzyl (3-(dimethyl(oxo)-l⁶-sulfaneylidene)-2-oxo-1-(spiro[3.3]heptan-2-yl)propyl)carbamate (0.400 g, 1.06 mmol, 1.00 eq) in THF (5.00 mL) was added LiBr (138 mg 1.59 mmol, 39.9 uL, 1.50 eq) and methane sulfonic acid (153 mg, 1.59 mmol, 113 μL, 1.50 eq). The reaction mixture was stirred at 45° C. for 1 h. The reaction mixture was diluted with water and extracted with ethyl acetate. The combined organic layers were dried over anhydrous Na₂SO₄, filtered and concentrated under reduced pressure to afford benzyl (3-bromo-2-oxo-1-(spiro[3.3]heptan-2-yl)propyl)carbamate (0.400 g, 1.05 mmol, 99% yield) as a yellow oil.

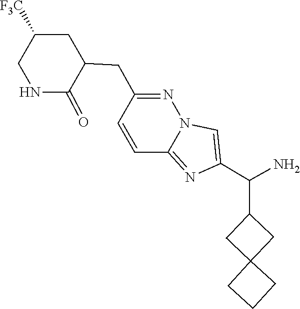 [SMILES: NC(c1cn2nc(CC3C[C@@H](C(F)(F)F)CNC3=O)ccc2n1)C1CC2(CCC2)C1]

(5R)-3-((2-(amino(spiro[3.3]heptan-2-yl)methyl)imidazo[1,2-b]pyridazin-6-yl)methyl)-5-(trifluoromethyl)piperidin-2-one was synthesized according to General Procedure 1, employing (5R)-3-((6-aminopyridazin-3-yl)methyl)-5-(trifluoromethyl)piperidin-2-one and benzyl (3-bromo-2-oxo-1-(spiro[3.3]heptan-2-yl)propyl)carbamate, and General Procedure 2, employing benzyl ((6-(((5R)-2-oxo-5-(trifluoromethyl)piperidin-3-yl)methyl)imidazo[1,2-b]pyridazin-2-yl)(spiro[3.3]heptan-2-yl)methyl)carbamate.

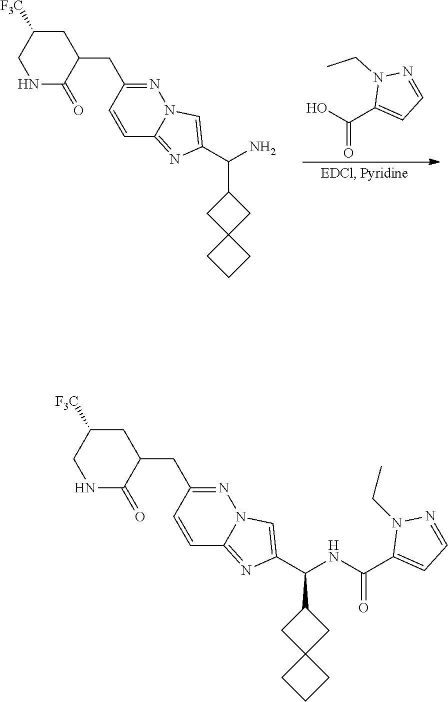

1-Ethyl-N-((1S)-(6-(((5R)-2-oxo-5-(trifluoromethyl)piperidin-3-yl)methyl)imidazo[1,2-b]pyridazin-2-yl)(spiro[3.3]heptan-2-yl)methyl)-1H-pyrazole-5-carboxamide was synthesized following the General Procedure 4 with (5R)-3-((2-(amino(spiro[3.3]heptan-2-yl)methyl)imidazo[1,2-b]pyridazin-6-yl)methyl)-5-(trifluoromethyl)piperidin-2-one (52.0 mg, 123 umol, 1.00 eq), 1-ethyl-1H-pyrazole-5-carboxylic acid (26.0 mg, 185 umol, 1.50 eq) and was purified by prep-TLC (SiO₂, DCM:MeOH) to afford 1-ethyl-N-((1S)-(6-(((5R)-2-oxo-5-(trifluoromethyl)piperidin-3-yl)methyl)imidazo[1,2-b]pyridazin-2-yl)(spiro[3.3]heptan-2-yl)methyl)-1H-pyrazole-5-carboxamide (40.0 mg, 72.7 umol, 59% yield) as a white solid. LCMS [M+H]+=544.3 m/z.

The title compound Compound 189 was isolated as the first eluting, single stereoisomer by chiral SFC purification (column: DAICEL CHIRALPAK IC (250 mm×30 mm, 10 μm); mobile phase: [0.1% NH₃H₂O ETOH]; B %: 60%-60%); (18.3 mg, 32.6 umol, 44% yield) and was obtained as a yellow solid. LCMS [M+H]⁺=544.3 m/z.

The title compound Compound 190 was isolated as the second eluting, single stereoisomer by chiral SFC purification (column: DAICEL CHIRALPAK AD (250 mm×30 mm, 10 μm); mobile phase: [0.1% NH₃H₂O ETOH]; B %: 40%-40%, 3.3 min); (20.0 mg, 36.0 umol, 49% yield) and was obtained as a yellow solid. LCMS [M+H]⁺=544.3 m/z.

### Example 189: Preparation of Compound 191

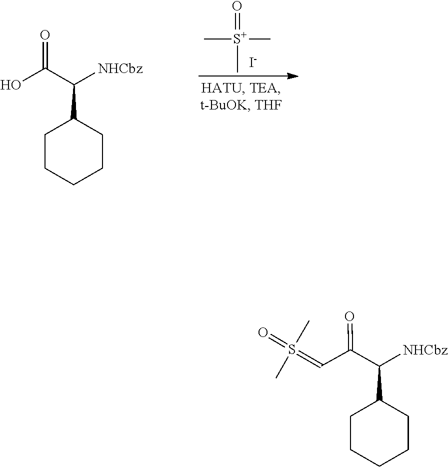

The preparation of mixture A: trimethylsulfoxonium iodide (2.27 g, 10.3 mmol, 3.00 eq) was added to a solution of t-BuOK (1.16 g, 10.3 mmol, 3.00 eq) in THF (10.0 mL) and the reaction mixture was stirred at 65° C. for 1 h under N₂. After that time, the solution was cooled to 0° C. to obtain the mixture A.

The preparation of mixture B: To a solution of (S)-2-(((benzyloxy)carbonyl)amino)-2-cyclohexylacetic acid (1.00 g, 3.43 mmol, 1.00 eq) in THE (10.0 mL) was added TEA (452 mg, 4.46 mmol, 621 μL, 1.30 eq) and HATU (1.70 g, 4.46 mmol, 1.30 eq) at 25° C. The reaction mixture was stirred at 25° C. for 1 h. The suspension was then filtered and the filtrate was cooled to 0° C. to obtain the mixture B.

The mixture B was added to the mixture A at 0° C. The resulting mixture was stirred at 25° C. for 2 h. The reaction mixture was quenched by the addition of water at 25° C., and extracted with ethyl acetate. The combined organic layers were dried over anhydrous Na₂SO₄, filtered and concentrated under reduced pressure to give a residue, which was purified by column chromatography (SiO₂, PE:EtOAc=0:1) to afford benzyl (S)-(1-cyclohexyl-3-(dimethyl(oxo)-l⁶-sulfaneylidene)-2-oxopropyl)carbamate (520 mg, 1.42 mmol, 42% yield) as a yellow gum. LCMS [M+H]+=366.2 m/z.

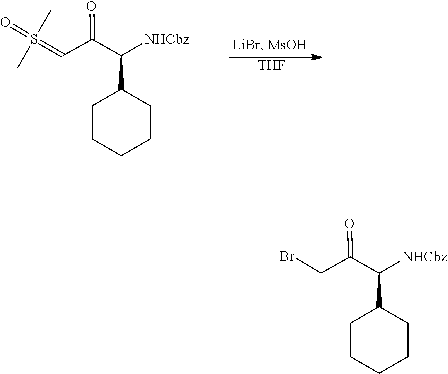 [SMILES: CS(C)(=O)=CC(=O)[C@@H](NC(=O)OCc1ccccc1)C1CCCCC1 ; O=C(N[C@H](C(=O)CBr)C1CCCCC1)OCc1ccccc1 ; "THF"]

To a solution of benzyl (S)-(1-cyclohexyl-3-(dimethyl(oxo)-l⁶-sulfaneylidene)-2-oxopropyl)carbamate (320 mg, 876 μmol, 1.00 eq) in THF (5.00 mL) was added LiBr (114 mg, 1.31 mmol, 33.0 μL, 1.50 eq) and methane sulfonic acid (126 mg, 1.31 mmol, 93.8 μL, 1.50 eq). The reaction mixture was stirred at 45° C. for 3 h. The reaction mixture was diluted with water and extracted with ethyl acetate. The combined organic layers were dried over anhydrous Na₂SO₄, filtered and concentrated under reduced pressure to afford benzyl (S)-(3-bromo-1-cyclohexyl-2-oxopropyl)carbamate (560 mg, crude) as a yellow gum.

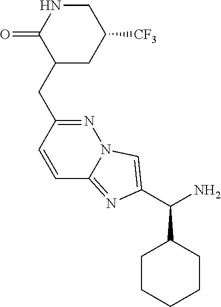 [SMILES: N[C@H](c1cn2nc(CC3C[C@@H](C(F)(F)F)CNC3=O)ccc2n1)C1CCCCC1]

(5R)-3-((2-((S)-amino(cyclohexyl)methyl)imidazo[1,2-b]pyridazin-6-yl)methyl)-5-(trifluoromethyl)piperidin-2-one was synthesized according to General Procedure 1, employing (5R)-3-((6-aminopyridazin-3-yl)methyl)-5-(trifluoromethyl)piperidin-2-one and benzyl (S)-(3-bromo-1-cyclohexyl-2-oxopropyl)carbamate, and General Procedure 2, employing benzyl ((1S)-cyclohexyl(6-(((5R)-2-oxo-5-(trifluoromethyl)piperidin-3-yl)methyl)imidazo[1,2-b]pyridazin-2-yl)methyl)carbamate.

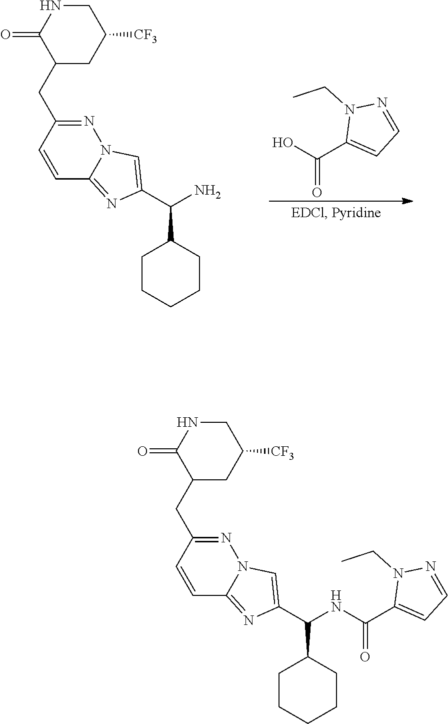

N-((1S)-cyclohexyl(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5R)-3-((2-((S)-amino(cyclohexyl)methyl)imidazo[1,2-b]pyridazin-6-yl)methyl)-5-(trifluoromethyl)piperidin-2-one (20.0 mg, 48.9 μmol, 1.00 eq), 1-ethyl-1H-pyrazole-5-carboxylic acid (8.21 mg, 58.6 μmol, 1.20 eq) and was purified by prep-HPLC (FA condition, column: Phenomenex luna C 18 150×25 mm, 10 μm; mobile phase: [water (FA)-ACN]; gradient: 34%-64% B) to afford Compound 191, N-((1S)-cyclohexyl(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide, (15.7 mg, 29.3 μmol, 60% yield) as a yellow solid. LCMS [M+H]⁺=532.2 m/z.

### Example 190: Preparation of Compound 192

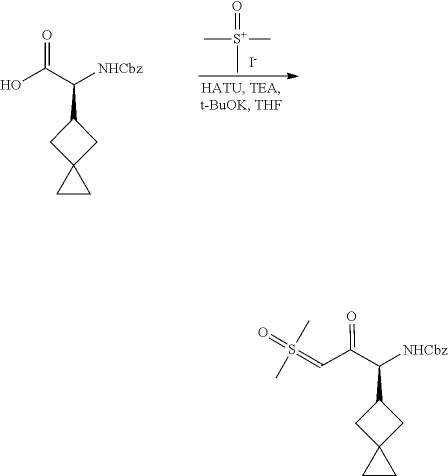

The preparation of mixture A: trimethylsulfoxonium iodide (798 mg, 3.63 mmol, 3.00 eq) was added to a solution of t-BuOK (407 mg, 3.63 mmol, 3.00 eq) in THF (10.0 mL) and the reaction mixture was stirred at 65° C. for 2 h under N₂. After that time, the solution was cooled to 0° C. to obtain the mixture A.

The preparation of mixture B: To a solution of (S)-2-(((benzyloxy)carbonyl)amino)-2-(spiro[2.3]hexan-5-yl)acetic acid (350 mg, 1.21 mmol, 1.00 eq) in THE (10.0 mL) was added TEA (159 mg, 1.57 mmol, 218 μL, 1.30 eq) and HATU (597 mg, 1.57 mmol, 1.30 eq) at 25° C. The reaction mixture was stirred at 25° C. for 2 h. The suspension was then filtered and the filtrate was cooled to 0° C. to obtain the mixture B.

The mixture B was added to the mixture A at 0° C. The resulting mixture was stirred at 25° C. for 1.5 h. The reaction mixture was quenched by the addition of water at 25° C., and extracted with DCM. The combined organic layers were dried over anhydrous Na₂SO₄, filtered and concentrated under reduced pressure to give a residue, which was purified by Prep-TLC (SiO₂, DCM:MeOH=10:1) to afford benzyl (S)-(3-(dimethyl(oxo)-l⁶-sulfaneylidene)-2-oxo-1-(spiro[2.3]hexan-5-yl)propyl)carbamate (180 mg, 495 μmol, 41% yield) as a yellow solid. LCMS [M+H]+=364.2 m/z.

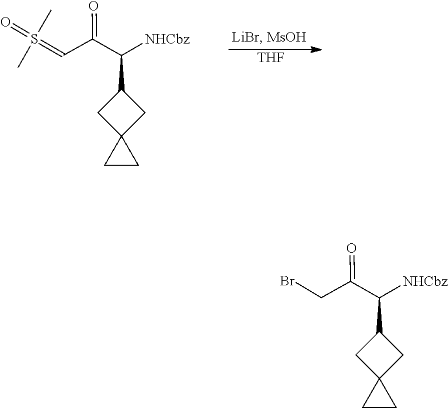 [SMILES: CS(C)(=O)=CC(=O)[C@@H](NC(=O)OCc1ccccc1)C1CC2(CC2)C1 ; O=C(N[C@H](C(=O)CBr)C1CC2(CC2)C1)OCc1ccccc1 ; "THF"]

To a solution of benzyl (S)-(3-(dimethyl(oxo)-l⁶-sulfaneylidene)-2-oxo-1-(spiro[2.3]hexan-5-yl)propyl)carbamate (180 mg, 495 μmol, 1.00 eq) in THF (2.00 mL) was added LiBr (129 mg, 1.49 mmol, 37.2 μL, 3.00 eq) and methane sulfonic acid (71.3 mg, 742 μmol, 53.0 μL, 1.50 eq). The reaction mixture was stirred at 45° C. for 1 h. The reaction mixture was diluted with water and extracted with DCM. The combined organic layers were dried over anhydrous Na₂SO₄, filtered and concentrated under reduced pressure to give a residue, which was purified by Prep-TLC (SiO₂, DCM:MeOH=10:1) to afford benzyl (S)-(3-bromo-2-oxo-1-(spiro[2.3]hexan-5-yl)propyl)carbamate (150 mg, 409 μmol, 83% yield) as a yellow solid. LCMS [M+H]+=366.1 m/z.

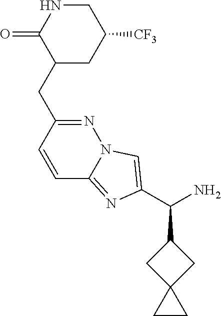 [SMILES: N[C@H](c1cn2nc(CC3C[C@@H](C(F)(F)F)CNC3=O)ccc2n1)C1CC2(CC2)C1]

(5R)-3-((2-((S)-amino(spiro[2.3]hexan-5-yl)methyl)imidazo[1,2-b]pyridazin-6-yl)methyl)-5-(trifluoromethyl)piperidin-2-one was synthesized according to General Procedure 1, employing (5R)-3-((6-aminopyridazin-3-yl)methyl)-5-(trifluoromethyl)piperidin-2-one and benzyl (S)-(3-bromo-2-oxo-1-(spiro[2.3]hexan-5-yl)propyl)carbamate, and General Procedure 3, employing benzyl ((1S)-(6-(((5R)-2-oxo-5-(trifluoromethyl)piperidin-3-yl)methyl)imidazo[1,2-b]pyridazin-2-yl)(spiro[2.3]hexan-5-yl)methyl)carbamate.

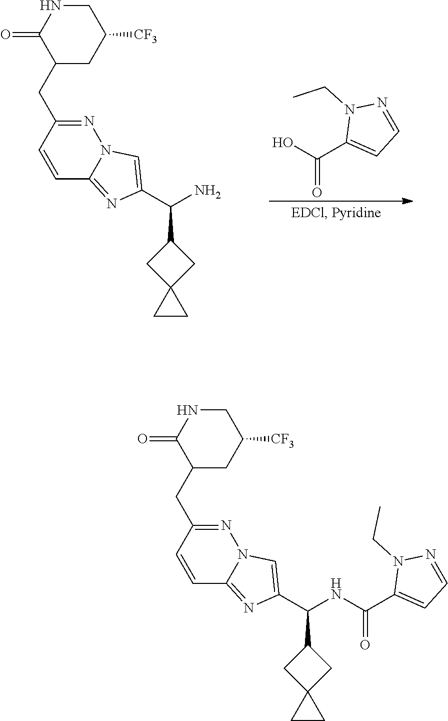

1-Ethyl-N-((1S)-(6-(((5R)-2-oxo-5-(trifluoromethyl)piperidin-3-yl)methyl)imidazo[1,2-b]pyridazin-2-yl)(spiro[2.3]hexan-5-yl)methyl)-1H-pyrazole-5-carboxamide was synthesized following the General Procedure 4 with (5R)-3-((2-((S)-amino(spiro[2.3]hexan-5-yl)methyl)imidazo[1,2-b]pyridazin-6-yl)methyl)-5-(trifluoromethyl)piperidin-2-one (40.0 mg, 98.1 μmol, 1.00 eq), 1-ethyl-1H-pyrazole-5-carboxylic acid (13.7 mg, 98.1 μmol, 1.00 eq) and was purified by prep-TLC (SiO₂, DCM:MeOH) to afford Compound 192, 1-ethyl-N—((1S)-(6-(((5R)-2-oxo-5-(trifluoromethyl)piperidin-3-yl)methyl)imidazo[1,2-b]pyridazin-2-yl)(spiro[2.3]hexan-5-yl)methyl)-1H-pyrazole-5-carboxamide, (14.83 mg, 26.2 μmol, 27% yield) as a yellow solid. LCMS [M+H]⁺=530.3 m/z.

### Example 191: X-Ray Crystallographic Analysis

### X-Ray Crystallographic Analysis of (3S,5R)-3-((6-aminopyridazin-3-yl)methyl)-5-(trifluoromethyl)piperidin-2-one

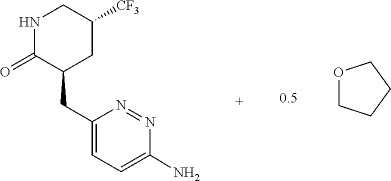 [SMILES: Nc1ccc(C[C@@H]2C[C@@H](C(F)(F)F)CNC2=O)nn1 ; C1CCOC1]

The absolute configuration of (3S,5R)-3-((6-aminopyridazin-3-yl)methyl)-5-(trifluoromethyl)piperidin-2-one was elucidated by single crystal X-ray diffraction. Crystals were grown by slowly evaporating a solution of a 2 mg sample in 200 μL of 1:1 MeCN/THF. [parameters: CuKα, orthorhombic space group=P2₁2₁2₁, a=5.65060(10) Å, b=13.6005(2) Å, c=38.8812(7) Å, α=90°, β=90°, γ=90°].

### X-Ray Crystallographic Analysis of (R)-3-((6-aminopyridazin-3-yl)methyl)piperidin-2-one

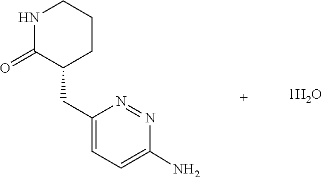 [SMILES: Nc1ccc(C[C@H]2CCCNC2=O)nn1]

The absolute configuration of (R)-3-((6-aminopyridazin-3-yl)methyl)piperidin-2-one was elucidated by single crystal X-ray diffraction. Crystals were grown by slowly evaporating a solution of a 2 mg sample in 200 μL of 1:1 DCM/MeOH. [parameters: CuKα, monoclinic space group=P2₁, a=9.9285(2) Å, b=12.3303(2) Å, c=10.2524(2) Å, α=90°, β=114.683(2)°, γ=90°].

### Example 192: Preparation of Compound 193

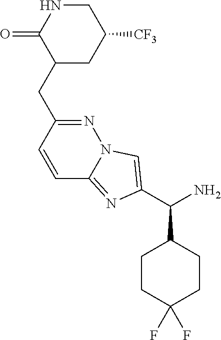 [SMILES: N[C@H](c1cn2nc(CC3C[C@@H](C(F)(F)F)CNC3=O)ccc2n1)C1CCC(F)(F)CC1]

(5R)-3-((2-((S)-amino(4,4-difluorocyclohexyl)methyl)imidazo[1,2-b]pyridazin-6-yl)methyl)-5-(trifluoromethyl)piperidin-2-one was synthesized according to General Procedure 1, employing (5R)-3-((6-aminopyridazin-3-yl)methyl)-5-(trifluoromethyl)piperidin-2-one and benzyl (S)-(3-bromo-1-(4,4-difluorocyclohexyl)-2-oxopropyl)carbamate, and General Procedure 3, employing benzyl ((1S)-(4,4-difluorocyclohexyl)(6-(((5R)-2-oxo-5-(trifluoromethyl)piperidin-3-yl)methyl)imidazo[1,2-b]pyridazin-2-yl)methyl)carbamate.

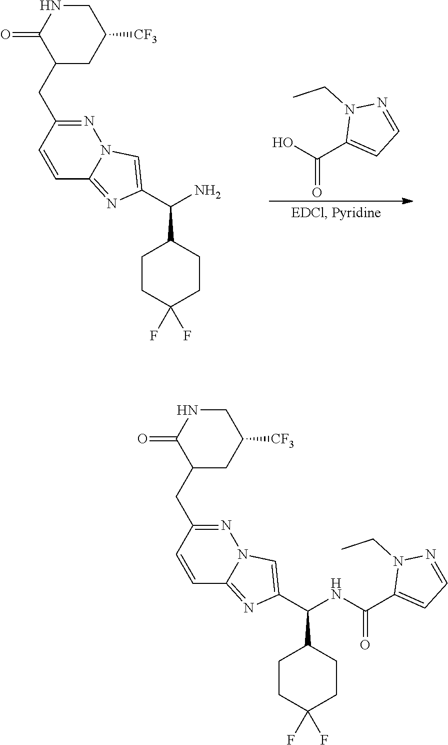

N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5R)-3-((2-((S)-amino(4,4-difluorocyclohexyl)methyl)imidazo[1,2-b]pyridazin-6-yl)methyl)-5-(trifluoromethyl)piperidin-2-one (50.0 mg, 112 umol, 1.00 eq), 1-ethyl-1H-pyrazole-5-carboxylic acid (23.6 mg, 168 umol, 1.50 eq) and was purified by prep-TLC (SiO₂, DCM:MeOH) to afford Compound 193 N-((1S)-(4,4-difluorocyclohexyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (25.72 mg, 43.28 umol, 39% yield) as a white solid. LCMS [M+H]⁺=568.2 m/z.

### Example 193: Preparation of Compound 194

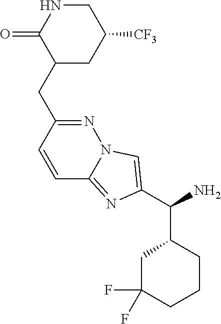 [SMILES: N[C@H](c1cn2nc(CC3C[C@@H](C(F)(F)F)CNC3=O)ccc2n1)[C@@H]1CCCC(F)(F)C1]

(5R)-3-((2-((S)-amino((R)-3,3-difluorocyclohexyl)methyl)imidazo[1,2-b]pyridazin-6-yl)methyl)-5-(trifluoromethyl)piperidin-2-one was synthesized according to General Procedure 1, employing (5R)-3-((6-aminopyridazin-3-yl)methyl)-5-(trifluoromethyl)piperidin-2-one and benzyl ((S)-3-bromo-1-((R)-3,3-difluorocyclohexyl)-2-oxopropyl)carbamate, and General Procedure 2, employing benzyl ((1S)—((R)-3,3-difluorocyclohexyl)(6-(((5R)-2-oxo-5-(trifluoromethyl)piperidin-3-yl)methyl)imidazo[1,2-b]pyridazin-2-yl)methyl)carbamate.

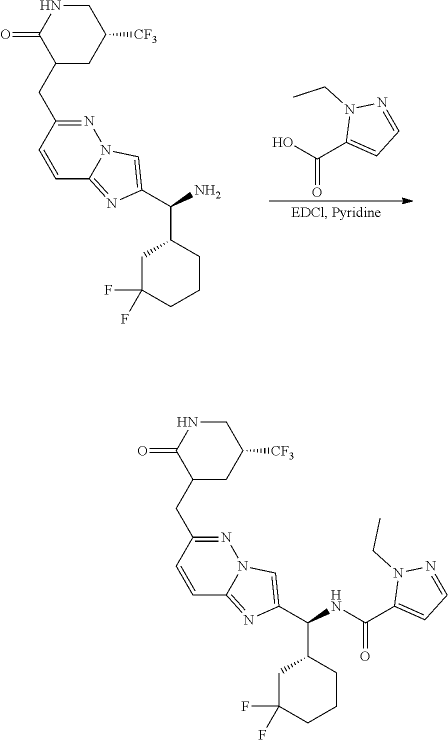

N-((1S)—((R)-3,3-difluorocyclohexyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide was synthesized following the General Procedure 4 with (5R)-3-((2-((S)-amino((R)-3,3-difluorocyclohexyl)methyl)imidazo[1,2-b]pyridazin-6-yl)methyl)-5-(trifluoromethyl)piperidin-2-one (70.0 mg, 157 μmol, 1.00 eq), 1-ethyl-1H-pyrazole-5-carboxylic acid (33.0 mg, 236 μmol, 1.50 eq) and was purified by prep-TLC (SiO₂, DCM:MeOH) to afford Compound 194 N-((1S)—((R)-3,3-difluorocyclohexyl)(6-(((5R)-2-oxo-5-(trifluoromethyl)piperidin-3-yl)methyl)imidazo[1,2-b]pyridazin-2-yl)methyl)-1-ethyl-1H-pyrazole-5-carboxamide (47.5 mg, 76.2 μmol, 49% yield) as a white solid. LCMS [M+H]+=568.2 m/z.

### Example 194: Preparation of Compound 195

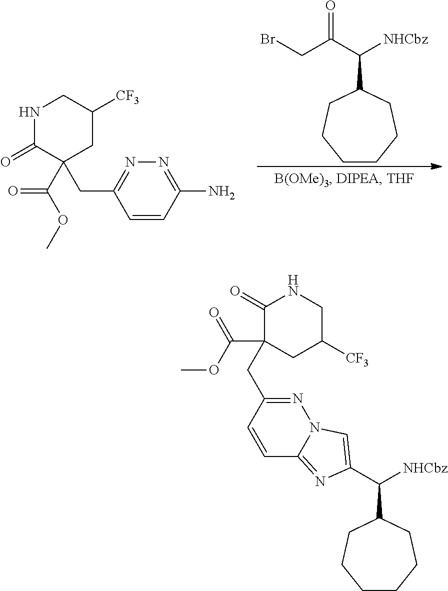 [SMILES: COC(=O)C1(Cc2ccc(N)nn2)CC(C(F)(F)F)CNC1=O ; O=C(N[C@H](C(=O)CBr)C1CCCCCC1)OCc1ccccc1 ; COC(=O)C1(Cc2ccc3nc([C@@H](NC(=O)OCc4ccccc4)C4CCCCCC4)cn3n2)CC(C(F)(F)F)CNC1=O]

To a solution of methyl 3-((6-aminopyridazin-3-yl)methyl)-2-oxo-5-(trifluoromethyl)piperidine-3-carboxylate (40.0 mg, 89.6 umol, 1.00 eq, TFA salt) and benzyl (S)-(3-bromo-1-cycloheptyl-2-oxopropyl)carbamate (55.2 mg, 144 umol, 1.61 eq) in THF (2.00 mL) was added B(OMe)₃ (46.5 mg, 448 umol, 50.6 uL, 5.00 eq) and DIEA (115 mg, 896 umol, 156 μL, 10.0 eq). The mixture was stirred at 70° C. for 2 h. The reaction mixture was diluted with water and extracted with ethyl acetate, dried over anhydrous Na₂SO₄, filtered and concentrated under reduced pressure to give a residue, which was purified by prep-TLC (SiO₂, EtOAc:PE, 2:1) to afford methyl 3-((2-((S)-(((benzyloxy)carbonyl)amino)(cycloheptyl)methyl)imidazo[1,2-b]pyridazin-6-yl)methyl)-2-oxo-5-(trifluoromethyl)piperidine-3-carboxylate (60.0 mg) as a yellow solid.

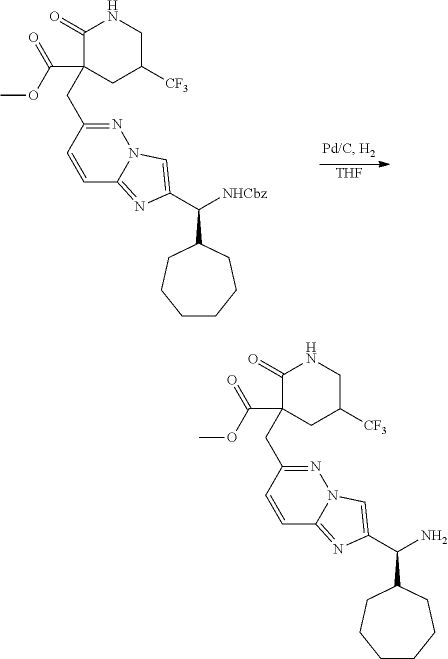 [SMILES: COC(=O)C1(Cc2ccc3nc([C@@H](NC(=O)OCc4ccccc4)C4CCCCCC4)cn3n2)CC(C(F)(F)F)CNC1=O ; COC(=O)C1(Cc2ccc3nc([C@@H](N)C4CCCCCC4)cn3n2)CC(C(F)(F)F)CNC1=O ; "THF"]

To a solution of methyl 3-((2-((S)-(((benzyloxy)carbonyl)amino)(cycloheptyl)methyl)imidazo[1,2-b]pyridazin-6-yl)methyl)-2-oxo-5-(trifluoromethyl)piperidine-3-carboxylate (60.0 mg, 97.4 umol, 1.00 eq) in THF (3.00 mL) was added Pd/C (40.0 mg, 10.0% purity) under N₂. The suspension was degassed under vacuum and purged with H₂ three times. The reaction mixture was stirred under H₂ (15.0 psi) at 18° C. for 5 h. The reaction mixture was filtered with MeOH and the filtrate was concentrated under reduced pressure to afford methyl 3-((2-((S)-amino(cycloheptyl)methyl)imidazo[1,2-b]pyridazin-6-yl)methyl)-2-oxo-5-(trifluoromethyl)piperidine-3-carboxylate (50.0 mg) as a yellow solid. LCMS [M+H]+=482.3 m/z.

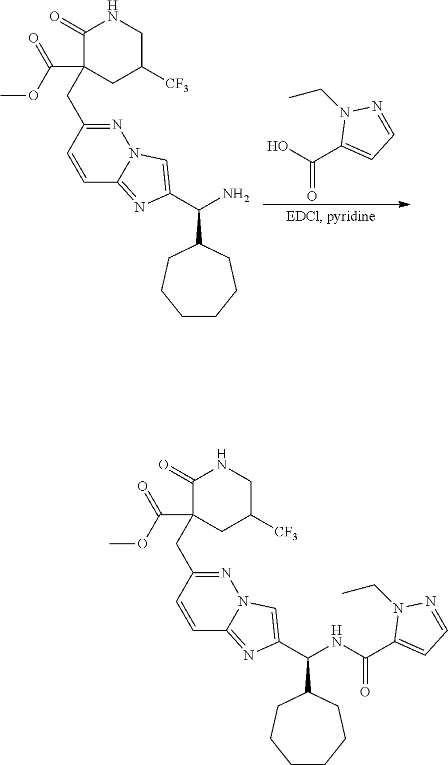

To a solution of methyl 3-((2-((S)-amino(cycloheptyl)methyl)imidazo[1,2-b]pyridazin-6-yl)methyl)-2-oxo-5-(trifluoromethyl)piperidine-3-carboxylate (40.0 mg, 83.0 umol, 1.00 eq) and 1-ethyl-1H-pyrazole-5-carboxylic acid (13.9 mg, 99.6 umol, 1.20 eq) in pyridine (1.00 mL) was added EDCI (63.7 mg, 332 umol, 4.00 eq). The reaction mixture was stirred at 18° C. for 2 h. The reaction mixture was diluted with water and extracted with ethyl acetate, dried over anhydrous Na₂SO₄, filtered and concentrated under reduced pressure to give a residue, which was purified by prep-TLC (SiO₂, ethyl acetate:MeOH, 18:1) to afford methyl 3-((2-((S)-cycloheptyl(1-ethyl-1H-pyrazole-5-carboxamido)methyl)imidazo[1,2-b]pyridazin-6-yl)methyl)-2-oxo-5-(trifluoromethyl)piperidine-3-carboxylate (45.0 mg, 74.5 umol, 90% yield) as a yellow solid. LCMS [M+H]+=604.4 m/z.

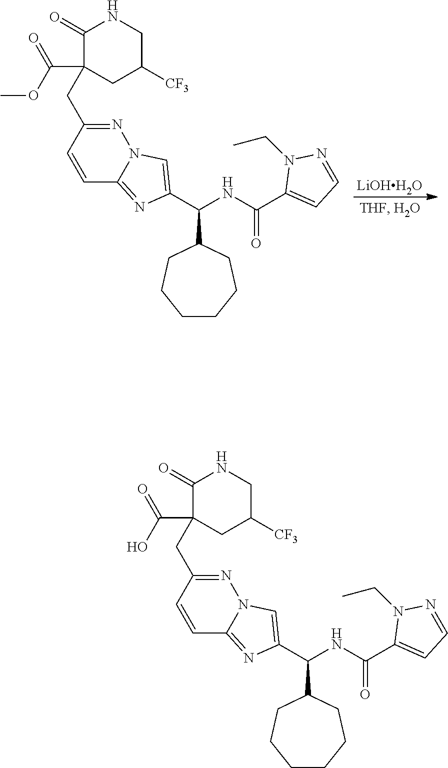

To a solution of methyl 3-((2-((S)-cycloheptyl(1-ethyl-1H-pyrazole-5-carboxamido)methyl)imidazo[1,2-b]pyridazin-6-yl)methyl)-2-oxo-5-(trifluoromethyl)piperidine-3-carboxylate (45.0 mg, 74.5 umol, 1.00 eq) in THF (2.00 mL) and water (0.50 mL) was added LiOH·H₂O (9.38 mg, 223 umol, 3.00 eq). The reaction mixture was stirred at 18° C. for 6 h. The reaction mixture was diluted with water, and a 1M aqueous solution of hydrochloric acid was added to adjust the pH to 4. The mixture was concentrated under reduced pressure to afford 3-((2-((S)-cycloheptyl(1-ethyl-1H-pyrazole-5-carboxamido)methyl)imidazo[1,2-b]pyridazin-6-yl)methyl)-2-oxo-5-(trifluoromethyl)piperidine-3-carboxylic acid (40.0 mg, 67.8 umol, 91% yield) as a white solid. LCMS [M+H]+=590.4 m/z.

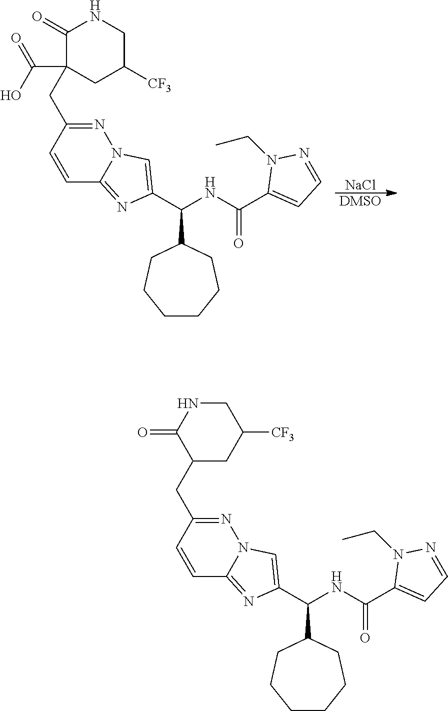

To a solution of 3-((2-((S)-cycloheptyl(1-ethyl-1H-pyrazole-5-carboxamido)methyl)imidazo[1,2-b]pyridazin-6-yl)methyl)-2-oxo-5-(trifluoromethyl)piperidine-3-carboxylic acid (40.0 mg, 67.8 umol, 1.00 eq) in DMSO (1.00 mL) was added NaCl (11.8 mg 203 umol, 3.00 eq). The reaction mixture was stirred at 120° C. for 1 h. The reaction mixture was concentrated under reduced pressure to give a residue, which was purified by prep-HPLC (column: Phenomenex luna C18 150×25 mm, 10 μm; mobile phase: [water (FA)-ACN]; B %: 41%-71%) to afford N-((1S)-cycloheptyl(6-((2-oxo-5-(trifluoromethyl)piperidin-3-yl)methyl)imidazo[1,2-b]pyridazin-2-yl)methyl)-1-ethyl-1H-pyrazole-5-carboxamide (20.14 mg 3 5.4 umol, 52% yield) as a white solid. LCMS [M+H]⁺=546.3 m/z.

Compound 195 was isolated as the fourth eluting, single stereoisomer by chiral SFC purification (column: Daicel Chiralpak IC (250 mm×30 mm, 10 μm); mobile phase: [0.1% NH₃H₂O in MeOH]; B %: 40%-40%); (3.94 mg, 7.22 umol, 22% yield) and was obtained as a brown solid. LCMS [M+H]⁺=546.3 m/z.

### Example 195: Preparation of Compounds 201 and 202

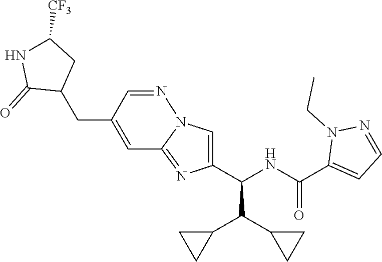

N-((1 S)-2,2-dicyclopropyl-1-(7-(((5S)-2-oxo-5-(trifluoromethyl)pyrrolidin-3-yl)methyl)imidazo[1,2-b]pyridazin-2-yl)ethyl)-1-ethyl-1H-pyrazole-5-carboxamide. (Compounds 201 and 202).

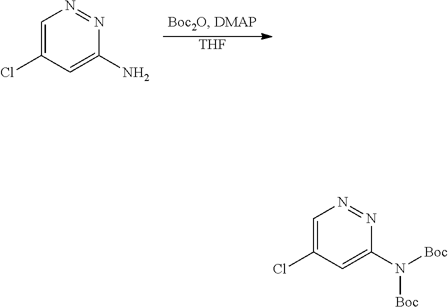 [SMILES: Nc1cc(Cl)cnn1 ; CC(C)(C)OC(=O)N(C(=O)OC(C)(C)C)c1cc(Cl)cnn1 ; "THF"]

To a solution of 5-chloropyridazin-3-amine (6.00 g, 46.3 mmol, 1.00 eq) in THF (60.0 mL) was added DMAP (1.13 g, 9.26 mmol, 0.200 eq) and di-tert-butyl dicarbonate (40.4 g, 185 mmol, 42.5 mL, 4.00 eq). The mixture was stirred at 15° C. for 12 h. The reaction mixture was then concentrated under reduced pressure. The residue was purified by column chromatography (SiO₂, PE:EtOAc=1:0 to 0:1) to afford tert-butyl (tert-butoxycarbonyl)(5-chloropyridazin-3-yl)carbamate (8.00 g, 24.2 mmol, 52% yield) as a white solid. LCMS [M+H]⁺=330.2 m/z.

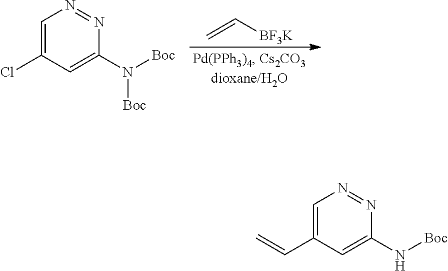

A mixture of tert-butyl (tert-butoxycarbonyl)(5-chloropyridazin-3-yl)carbamate (8.00 g 24.2 mmol, 1.00 eq), potassium trifluoro(vinyl)borate (32.4 g, 242 mmol, 10.0 eq), Pd(PPh₃)₄ (2.80 g, 2.43 mmol, 0.100 eq), and Cs₂CO₃ (15.8 g, 48.5 mmol, 2.00 eq) in dioxane (60.0 mL) and H₂O (30.0 mL) was degassed and purged with nitrogen gas three times before stirring at 80° C. for 12 h. The reaction mixture was diluted with saturated aqueous NaHCO₃, extracted with EtOAc, dried with anhydrous Na₂SO₄, filtered, and concentrated under reduced pressure. The residue was purified by column chromatography (SiO₂, PE:EtOAc=1:0 to 0:1) to afford tert-butyl (5-vinylpyridazin-3-yl)carbamate (3.00 g, 13.5 mmol, 56% yield) as a yellow oil. LCMS [M+H]⁺=226.0 m/z.

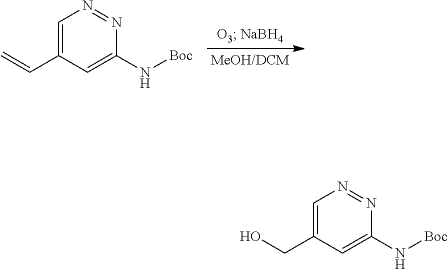 [SMILES: C=Cc1cnnc(NC(=O)OC(C)(C)C)c1 ; CC(C)(C)OC(=O)Nc1cc(CO)cnn1]

Gaseous O₃ was bubbled into a solution of tert-butyl (5-vinylpyridazin-3-yl)carbamate (3.00 g, 13.5 mmol, 1.00 eq) in DCM (30.0 mL) and MeOH (10.0 mL) at −78° C. for 30 min. Then NaBH₄ (4.10 g, 108 mmol, 8.00 eq) was added portionwise at 0° C. The resulting mixture was stirred at 0° C. for 1.5 h. The residue was diluted with NH₄Cl, extracted with EtOAc, dried over Na₂SO₄, filtered, and concentrated under reduced pressure. The residue was purified by column chromatography (SiO₂, PE:EtOAc=1:0 to 0:1) followed by prep-TLC (SiO₂, DCM:MeOH=10:1) to afford tert-butyl (5-(hydroxymethyl)pyridazin-3-yl)carbamate (600 mg, 2.66 mmol, 20% yield) as a white solid. LCMS [M+H]⁺ 226.0 m/z.

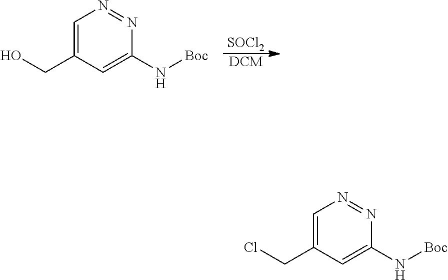

A solution of tert-butyl (5-(hydroxymethyl)pyridazin-3-yl)carbamate (400 mg, 1.78 mmol, 1.00 eq) and SOCl₂ (422 mg, 3.55 mmol, 257 μL, 2.00 eq) in DCM (4.00 mL) was stirred at 0° C. for 1 h. The reaction mixture was concentrated under reduced pressure. The crude product tert-butyl (5-(chloromethyl)pyridazin-3-yl)carbamate (400 mg, 1.64 mmol, 92% yield) was obtained as a yellow solid, which was used directly in the next step. LCMS [M+H]⁺ 244.0 m/z.

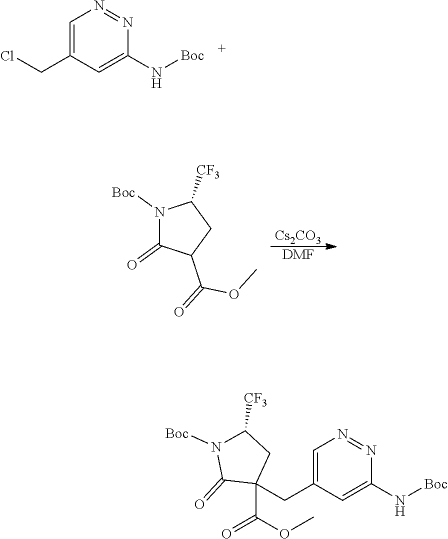

To a solution of tert-butyl (5-(chloromethyl)pyridazin-3-yl)carbamate (400 mg, 1.64 mmol, 1.00 eq) and 1-(tert-butyl) 3-methyl (5S)-2-oxo-5-(trifluoromethyl)pyrrolidine-1,3-dicarboxylate (561 mg, 1.81 mmol, 1.10 eq) in DMF (10.0 mL) was added Cs₂CO₃ (1.07 g, 3.28 mmol, 2.00 eq). The mixture was stirred at 55° C. for 2 h. The reaction mixture was diluted with saturated aqueous NaHCO₃ and extracted with EtOAc, dried over Na₂SO₄, filtered, and concentrated under reduced pressure. The residue was purified by prep-HPLC (FA condition; column: Phenomenex Luna C18 (150*40 mm, 15 μm); mobile phase: [water (FA)-ACN]; B %: 43%-73%, 10 min) to afford 1-(tert-butyl) 3-methyl (5S)-3-((6-((tert-butoxycarbonyl)amino)pyridazin-4-yl)methyl)-2-oxo-5-(trifluoromethyl)pyrrolidine-1,3-dicarboxylate (400 mg, 771 μmol, 47% yield) as a yellow solid. LCMS [M+H]⁺=519.3 m/z.

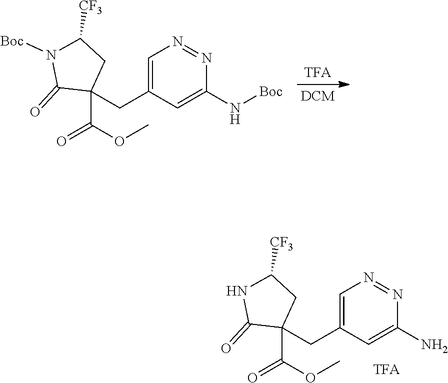 [SMILES: COC(=O)C1(Cc2cnnc(NC(=O)OC(C)(C)C)c2)C[C@@H](C(F)(F)F)N(C(=O)OC(C)(C)C)C1=O ; COC(=O)C1(Cc2cnnc(N)c2)C[C@@H](C(F)(F)F)NC1=O]

To a solution of 1-(tert-butyl) 3-methyl (5S)-3-((6-((tert-butoxycarbonyl)amino)pyridazin-4-yl)methyl)-2-oxo-5-(trifluoromethyl)pyrrolidine-1,3-dicarboxylate (400 mg, 771 μmol, 1.00 eq) in DCM (10.0 mL) was added TFA (175 mg, 1.54 mmol, 114 μL, 2.00 eq). The mixture was stirred at 15° C. for 2 h. The reaction mixture was concentrated under reduced pressure to afford methyl (5 S)-3-((6-aminopyridazin-4-yl)methyl)-2-oxo-5-(trifluoromethyl)pyrrolidine-3-carboxylate (300 mg, 694 μmol, TFA salt, 90% yield) as a yellow solid, which was used without further purification in the next step. LCMS [M+H]⁺=319.2

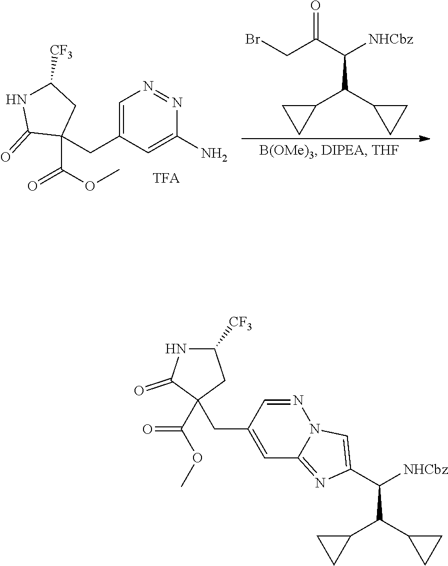 [SMILES: COC(=O)C1(Cc2cnnc(N)c2)C[C@@H](C(F)(F)F)NC1=O ; O=C(N[C@H](C(=O)CBr)C(C1CC1)C1CC1)OCc1ccccc1 ; COC(=O)C1(Cc2cnn3cc([C@@H](NC(=O)OCc4ccccc4)C(C4CC4)C4CC4)nc3c2)C[C@@H](C(F)(F)F)NC1=O]

To a solution of methyl (5S)-3-((6-aminopyridazin-4-yl)methyl)-2-oxo-5-(trifluoromethyl)pyrrolidine-3-carboxylate (80.0 mg, 251 μmol, 1.00 eq, TFA salt) and benzyl (S)-(4-bromo-1,1-dicyclopropyl-3-oxobutan-2-yl)carbamate (Int. 1, 114 mg, 301 μmol, 1.20 eq) in THE (5.00 mL) was added B(OMe)₃ (130 mg, 1.26 mmol, 141 μL, 5.00 eq) and DIEA (162 mg, 1.26 mmol, 218 μL, 5.00 eq). The mixture was stirred at 80° C. for 12 h. The reaction mixture was diluted with saturated aqueous NaHCO₃ and extracted with EtOAc, then dried over Na₂SO₄, filtered, and concentrated under reduced pressure. The residue was purified by prep-TLC (SiO₂, petroleum ether:EtOAc=1:9) to afford methyl (5S)-3-((2-((S)-1-(((benzyloxy)carbonyl)amino)-2,2-dicyclopropylethyl)imidazo[1,2-b]pyridazin-7-yl)methyl)-2-oxo-5-(trifluoromethyl)pyrrolidine-3-carboxylate (100 mg, 167 μmol, 67% yield) as a red solid. LCMS [M+H]⁺=600.3 m/z.

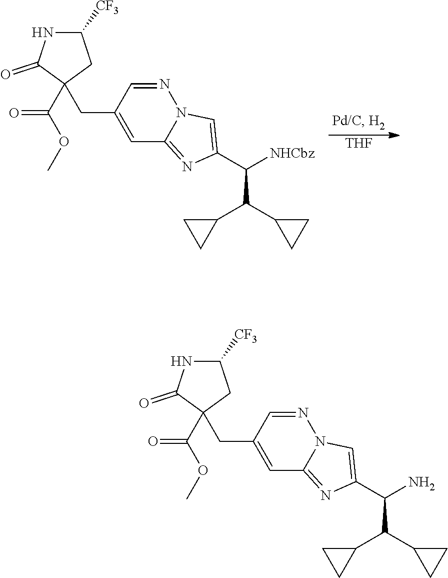 [SMILES: COC(=O)C1(Cc2cnn3cc([C@@H](NC(=O)OCc4ccccc4)C(C4CC4)C4CC4)nc3c2)C[C@@H](C(F)(F)F)NC1=O ; COC(=O)C1(Cc2cnn3cc([C@@H](N)C(C4CC4)C4CC4)nc3c2)C[C@@H](C(F)(F)F)NC1=O ; "THF"]

To a solution of methyl (5S)-3-((2-((S)-1-(((benzyloxy)carbonyl)amino)-2,2-dicyclopropylethyl)imidazo[1,2-b]pyridazin-7-yl)methyl)-2-oxo-5-(trifluoromethyl)pyrrolidine-3-carboxylate (150 mg, 250 μmol, 1.00 eq) in THF (5.00 mL) was added Pd/C (20.0 mg, 10.0% purity) under N₂. The suspension was degassed and purged with H₂ several times. The mixture was stirred under H₂ (15 psi) at 15° C. for 12 h. The reaction mixture was filtered, the filtrate was diluted with MeOH, and the mixture was filtered again. The reaction solution was concentrated under reduced pressure to afford methyl (5S)-3-((2-((S)-1-amino-2,2-dicyclopropylethyl)imidazo[1,2-b]pyridazin-7-yl)methyl)-2-oxo-5-(trifluoromethyl)pyrrolidine-3-carboxylate (100 mg, 214 μmol, 86% yield) as a yellow solid, which was used without further purification in the next reaction. LCMS [M+H]⁺=466.3 m/z.

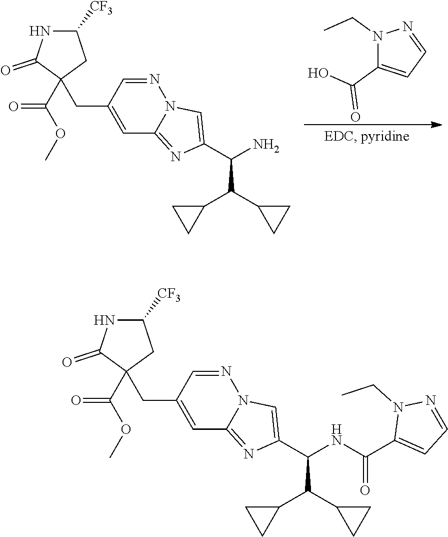

To a solution of methyl (5S)-3-((2-((S)-1-amino-2,2-dicyclopropylethyl)imidazo[1,2-b]pyridazin-7-yl)methyl)-2-oxo-5-(trifluoromethyl)pyrrolidine-3-carboxylate (100 mg, 214 μmol, 1.00 eq) and 1-ethyl-1H-pyrazole-5-carboxylic acid (36.1 mg, 257 μmol, 1.20 eq) in pyridine (2.00 mL) was added EDCI (82.4 mg, 429 μmol, 2.00 eq). The mixture was stirred at 15° C. for 2 h. The reaction mixture was diluted with saturated aqueous NaHCO₃ and extracted with EtOAc then dried over Na₂SO₄, filtered, and concentrated under reduced pressure. The residue was purified by prep-TLC (SiO₂, DCM:MeOH=10:1) to afford methyl (5S)-3-((2-((S)-2,2-dicyclopropyl-1-(1-ethyl-1H-pyrazole-5-carboxamido)ethyl)imidazo[1,2-b]pyridazin-7-yl)methyl)-2-oxo-5-(trifluoromethyl)pyrrolidine-3-carboxylate (80.0 mg, 136 μmol, 64% yield) as a yellow solid. LCMS [M+H]⁺=588.3 m/z.

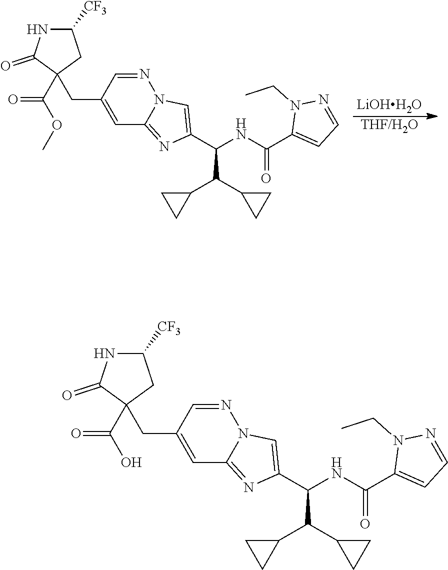

To a solution of methyl (5S)-3-((2-((S)-2,2-dicyclopropyl-1-(1-ethyl-1H-pyrazole-5-carboxamido)ethyl)imidazo[1,2-b]pyridazin-7-yl)methyl)-2-oxo-5-(trifluoromethyl)pyrrolidine-3-carboxylate (80.0 mg, 136 μmol, 1.00 eq) in THF (3.00 mL) and H₂O (3.00 mL) was added LiOH·H2O (11.4 mg, 272 μmol, 2.00 eq) at 0° C. The mixture was stirred at 15° C. for 2 h. The reaction mixture was diluted with 1.00 M HCl to pH=4. The reaction mixture was extracted with EtOAc, dried over Na₂SO₄, filtered, and concentrated under reduced pressure to afford (5 S)-3-((2—((S)-2,2-dicyclopropyl-1-(1-ethyl-1H-pyrazole-5-carboxamido)ethyl)imidazo[1,2-b]pyridazin-7-yl)methyl)-2-oxo-5-(trifluoromethyl)pyrrolidine-3-carboxylic acid (75.0 mg, 130 μmol, 96% yield) as a white solid. LCMS [M+H]⁺=574.3 m/z.

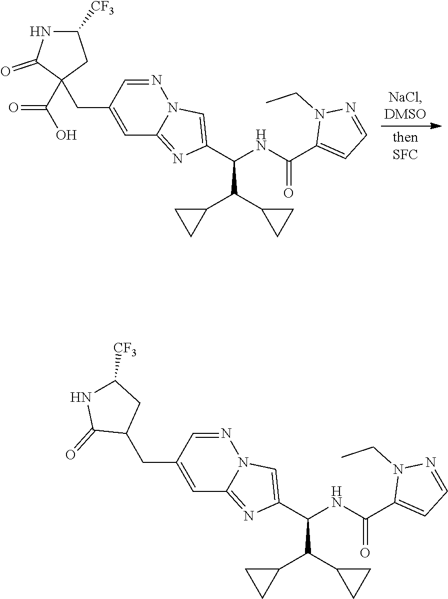 [SMILES: CCn1nccc1C(=O)N[C@H](c1cn2ncc(CC3(C(=O)O)C[C@@H](C(F)(F)F)NC3=O)cc2n1)C(C1CC1)C1CC1 ; CCn1nccc1C(=O)N[C@H](c1cn2ncc(CC3C[C@@H](C(F)(F)F)NC3=O)cc2n1)C(C1CC1)C1CC1]

To a solution of (5S)-3-((2-((S)-2,2-dicyclopropyl-1-(1-ethyl-1H-pyrazole-5-carboxamido)ethyl)imidazo[1,2-b]pyridazin-7-yl)methyl)-2-oxo-5-(trifluoromethyl)pyrrolidine-3-carboxylic acid (75.0 mg, 130 μmol, 1.00 eq) in DMSO (2.00 mL) was added NaCl (15.2 mg 261 μmol, 2.00 eq). The mixture was stirred at 120° C. for 2 h. The residue was diluted with H₂O, extracted with EtOAc, dried over Na₂SO₄, filtered, and concentrated under reduced pressure. The residue was purified by prep-TLC (SiO₂, DCM:MeOH=10:1). The mixture of diastereomers obtained was then purified by prep-SFC (Rt (P1)=0.926, Rt (P2)=1.066, column: DAICEL CHIRALCEL OJ (250 mm*30 mm, 10 μm); mobile phase: [0.1% NH₃H₂O MeOH]; B %: 15%-15%, 5.2 min).

Compound 201: The title compound, N—((S)-2,2-dicyclopropyl-1-(7-(((3R,5S)-2-oxo-5-(trifluoromethyl)pyrrolidin-3-yl)methyl)imidazo[1,2-b]pyridazin-2-yl)ethyl)-1-ethyl-1H-pyrazole-5-carboxamide, was obtained as the first eluting isomer (5.30 mg, 9.27 μmol, 7.1% yield) as a yellow solid. LCMS [M+H]⁺=530.4 m/z.

Compound 202: The title compound, N—((S)-2,2-dicyclopropyl-1-(7-(((3R,5S)-2-oxo-5-(trifluoromethyl)pyrrolidin-3-yl)methyl)imidazo[1,2-b]pyridazin-2-yl)ethyl)-1-ethyl-1H-pyrazole-5-carboxamide, was obtained as the second eluting isomer (5.30 mg, 9.27 μmol, 7.1% yield) as a yellow solid. LCMS [M+H]⁺=530.4 m/z.

### Example 196: Preparation of Compound 203

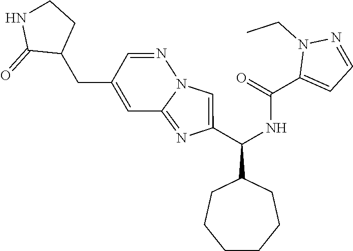

### N-((1 S)-cycloheptyl(7-((2-oxopyrrolidin-3-yl)methyl)imidazo[1,2-b]pyridazin-2-yl)methyl)-1-ethyl-1H-pyrazole-5-carboxamide. (Compound 203)

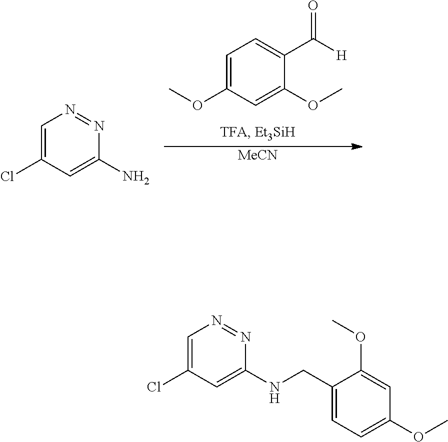 [SMILES: "MeCN" ; COc1ccc(C=O)c(OC)c1 ; Nc1cc(Cl)cnn1 ; COc1ccc(CNc2cc(Cl)cnn2)c(OC)c1]

To a solution of 5-chloropyridazin-3-amine (5 g, 38.6 mmol) and 2,4-dimethoxybenzaldehyde (19.6 g, 116 mmol, 3.0 eq) in ACN (420 mL) under nitrogen were added TFA (14.2 mL, 185 mmol, 4.8 eq) and triethylsilane (10.1 mL, 130 mmol, 3.37 eq) at RT. The mixture was heated at 100° C. and stirred for 2 h. The reaction mixture pH was adjusted to 9 by the addition of aqueous solution of 1 N NaOH. The reaction solution was extracted with EtOAc, dried over Na₂SO₄, filtered, and concentrated under vacuum. The residue was purified by column chromatography (SiO₂, PE:EtOAc=1:0 to 0:1) to afford 5-chloro-N-(2,4-dimethoxybenzyl)pyridazin-3-amine as an off-white solid (4.97 g, 17.8 mmol 46% yield). LCMS [M+H]⁺=280.1 m/z.

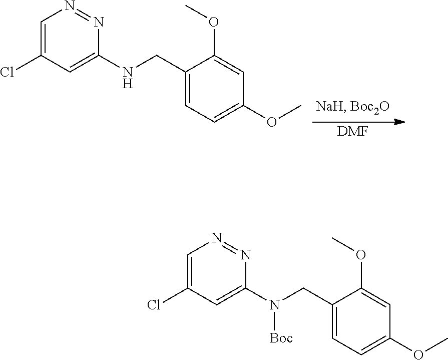

To a solution of 5-chloro-N-(2,4-dimethoxybenzyl)pyridazin-3-amine (4.97 g, 17.8 mmol, 1.0 eq) in DMF (104 mL) under nitrogen at 0° C. was added NaH (512 mg, 21.3 mmol, 1.2 eq). The mixture was stirred at 0° C. for 5 min. Di-tert-butyl dicarbonate (5.04 g, 23.1 mmol, 1.3 eq) was then added, and the reaction mixture was stirred at RT for 72 h. The reaction mixture was quenched with H₂O and extracted with EtOAc, dried over Na₂SO₄, filtered, and concentrated under vacuum. The residue was purified by column chromatography (SiO₂, PE:EtOAc=1:0 to 0:1) to afford tert-butyl (5-chloropyridazin-3-yl)(2,4-dimethoxybenzyl)carbamate as a yellow oil (5.93 g, 25.6 mmol, 88% yield). LCMS [M+H]⁺=380.1 m/z.

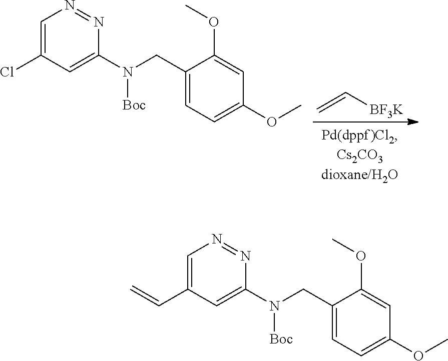

tert-Butyl (5-chloropyridazin-3-yl)(2,4-dimethoxybenzyl)carbamate (2.76 g, 7.27 mmol), Pd(dppf)Cl₂ (319 mg, 436 μmol, 0.06 eq), potassium vinyltrifluoroborate (1.56 g, 11.6 mmol, 1.6 eq), and Cs₂CO₃ (7.10 g, 21.8 mmol, 3 eq) were combined in a flask and placed under vacuum for 15 min and backfilled with nitrogen gas. A mix of dioxane (60 mL) and water (8 mL), previously sparged with nitrogen for 30 min, was then added. The resulting reaction mixture was heated at 90° C. for 90 h. The reaction mixture was concentrated under vacuum, and the residue was purified by column chromatography (SiO₂, PE:EtOAc=1:0 to 0:1) to afford tert-butyl (2,4-dimethoxybenzyl)(5-vinylpyridazin-3-yl)carbamate as an orange oil (2.21 g, 5.95 mmol, 82% yield). LCMS [M+H]⁺=372.1 m/z.

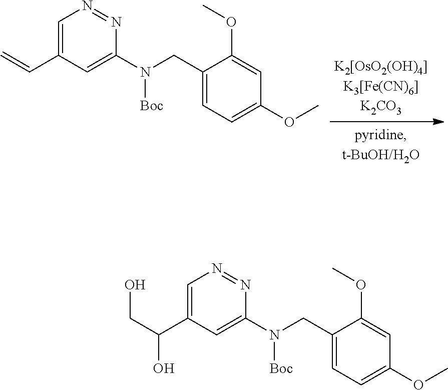 [SMILES: C=Cc1cnnc(N(Cc2ccc(OC)cc2OC)C(=O)OC(C)(C)C)c1 ; COc1ccc(CN(C(=O)OC(C)(C)C)c2cc(C(O)CO)cnn2)c(OC)c1]

tert-Butyl (2,4-dimethoxybenzyl)(5-vinylpyridazin-3-yl)carbamate (2.35 g, 6.32 mmol), K₂[OsO₂(OH)₄](4.84 mg, 13.1 umol), K₃[Fe(CN)₆](6.30 g, 19.1 mmol, 3.0 eq), and pyridine (5 μL, 63.2 μmol, 0.01 eq) were suspended in a mix of t-BuOH (13.1 mL) and water (13.1 mL) under nitrogen. The reaction mixture was stirred vigorously at RT for 24 h. After 24 h, additional K₂[OsO₂(OH)₄](4.84 mg, 13.1 umol) was added, and the reaction mixture was stirred for another 24 h at RT. Further charging of the reaction vessel with K₂[OsO₂(OH)₄](4.84 mg, 13.1 umol) was repeated one more time followed again by stirring for 24 h at RT. The reaction mixture was treated with saturated aqueous sodium bisulfite until effervescence ceased. The aqueous layer was then extracted with EtOAc five times. The combined organic layers were dried over Na₂SO₄, filtered, and concentrated under vacuum. The residue was purified by column chromatography (SiO₂, DCM:MeOH=1:0 to 85:15) to afford tert-butyl (5-(1,2-dihydroxyethyl)pyridazin-3-yl)(2,4-dimethoxybenzyl)carbamate as a yellow solid (1.57 g, 3.87 mmol, 61%). LCMS [M+H]⁺=406.2 m/z.

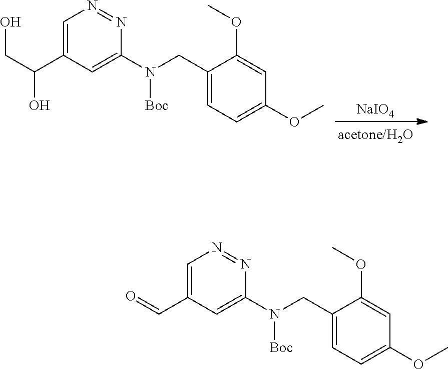

To a solution of tert-butyl (5-(1,2-dihydroxyethyl)pyridazin-3-yl)(2,4-dimethoxybenzyl) carbamate (2.00 g, 4.93 mmol, 1 eq) in a solution of acetone (26 mL) and water (13 mL) was added NaIO₄ (1.06 g, 4.93 mmol, 1 eq). The reaction mixture was stirred at RT for 2 h. The reaction mixture was treated with brine and extracted with EtOAc. The combined organic layers were dried over Na₂SO₄, filtered, and concentrated under vacuum to afford tert-butyl (2,4-dimethoxybenzyl)(5-formylpyridazin-3-yl)carbamate as a yellow solid (1.64 g, 4.4 mmol, 89%). The crude material was used without further purification.

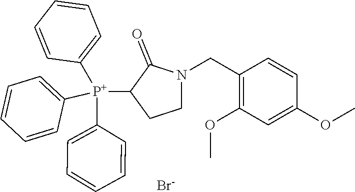 [SMILES: COc1ccc(CN2CCC([P+](c3ccccc3)(c3ccccc3)c3ccccc3)C2=O)c(OC)c1 ; [Br-]]

### (1-(2,4-dimethoxybenzyl)-2-oxopyrrolidin-3-yl)triphenylphosphonium bromide

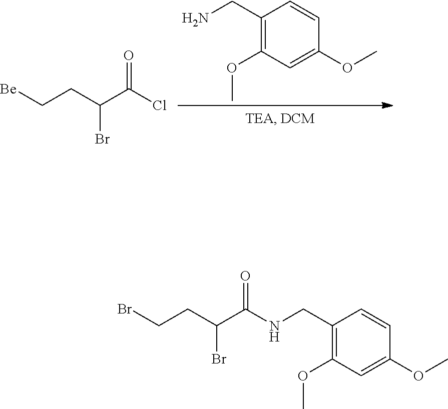

To a solution of 2,4-dibromobutanoyl chloride (12.6 g, 75.6 mmol, 11.4 mL, 1.00 eq) and TEA (9.49 g, 93.8 mmol, 13.0 mL, 1.24 eq) in DCM (150 mL) was added (2,4-dimethoxyphenyl)methanamine (20.0 g, 75.6 mmol, 10.0 mL, 1.00 eq) in DCM (100 mL) at 0° C. The mixture was stirred at 0° C. for 2 h. The reaction mixture was diluted with H₂O and extracted with DCM. The combined organic layers were washed with brine three times, dried over Na₂SO₄, filtered, and concentrated under reduced pressure to give a residue. The residue was purified by column chromatography (SiO₂, PE:EtOAc=1:0 to 0:1). 2,4-dibromo-N-(2,4-dimethoxybenzyl)butanamide (25.0 g, 63.2 mmol, 84% yield) was obtained as a white solid. ¹H-NMR: δ 7.18 (t, J=8.0 Hz, 1H), 6.85-6.75 (m, 1H), 6.50-6.30 (m, 2H), 4.50-4.48 (m, 1H), 4.48-4.46 (m, 2H), 3.85 (s, 3H), 3.81 (s, 3H), 3.70-3.50 (m, 2H), 2.80-2.65 (m, 1H), 2.55-2.30 (m, 1H).

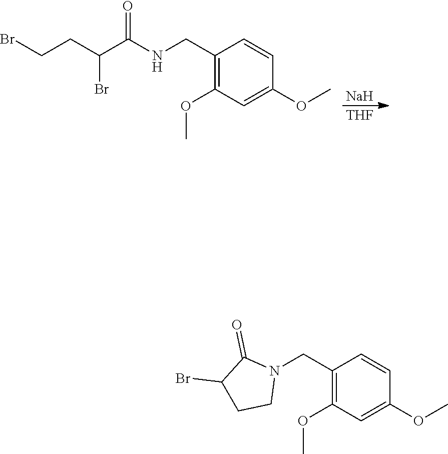

To a solution of 2,4-dibromo-N-(2,4-dimethoxybenzyl)butanamide (25.0 g, 63.2 mmol, 1.00 eq) in THF (750 mL) was added NaH (5.06 g, 126 mmol, 60.0% purity, 2.00 eq) at 0° C. The mixture was stirred at RT for 1 h. The reaction mixture was diluted with H₂O and extracted with DCM. The combined organic layers were washed with brine three times, dried over Na₂SO₄, filtered, and concentrated under reduced pressure to give desired product, 3-bromo-1-(2,4-dimethoxybenzyl)pyrrolidin-2-one (18.0 g, 57.2 mmol, 91% yield).

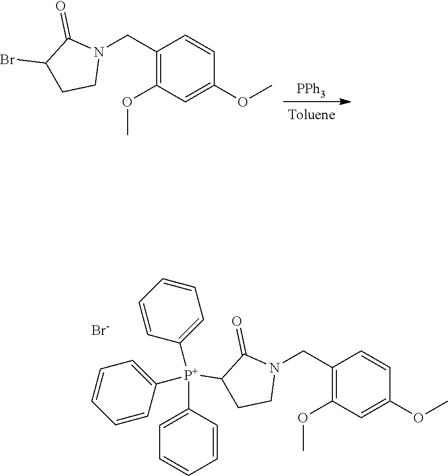 [SMILES: COc1ccc(CN2CCC(Br)C2=O)c(OC)c1 ; COc1ccc(CN2CCC([P+](c3ccccc3)(c3ccccc3)c3ccccc3)C2=O)c(OC)c1 ; [Br-] ; "PPh3"]

To a solution of 3-bromo-1-(2,4-dimethoxybenzyl)pyrrolidin-2-one (18.0 g, 57.2 mmol, 1.00 eq) in toluene (180 mL) was added PPh₃ (15.7 g, 60.1 mmol, 1.05 eq). The mixture was stirred at 100° C. for 6 h. The reaction mixture was concentrated under reduced pressure and the crude product was triturated with EtOAc at RT for 5 min. Desired product, (1-(2,4-dimethoxybenzyl)-2-oxopyrrolidin-3-yl)triphenylphosphonium bromide (18.0 g, 58% purity, 3 1% yield), was obtained as a white solid by filtration. The crude product was used without further purification. ¹H-NMR: δ 7.91-7.80 (m, 8H), 7.75-7.70 (m, 7H), 6.83 (d, J=8.4 Hz, 1H), 6.60-6.50 (m, 1H), 6.50-6.35 (m, 2H), 5.40-5.25 (m, 1H), 4.40-4.20 (m, 2H), 3.80-3.75 (m, 3H), 3.71-3.70 (m, 3H), 3.55-3.40 (m, 1H), 3.25-3.15 (m, 1H), 2.75-2.63 (m, 1H), 2.40-2.33 (m, 1H). LCMS [M+H]⁺=496.2 m/z.

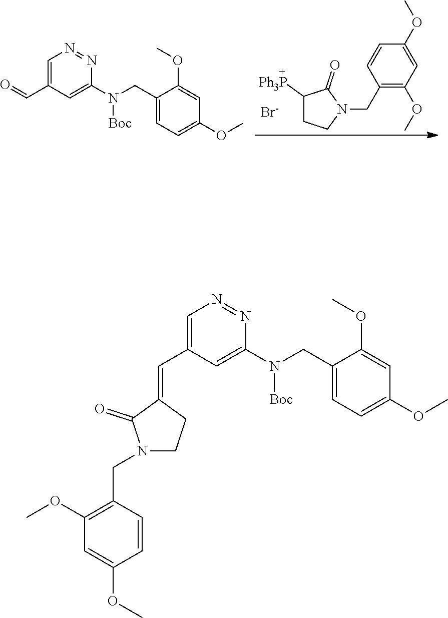

To a solution of (1-(2,4-dimethoxybenzyl)-2-oxopyrrolidin-3-yl)triphenylphosphonium bromide (1.39 g, 2.31 mmol, 1.00 eq) in THF (17 mL) under nitrogen at 0° C. was added potassium t-butoxide (2.32 mL, 2.32 mmol, 1 M in THF, 1.00 eq). The mixture was stirred at 0° C. for 30 min. A solution of tert-butyl (2,4-dimethoxybenzyl)(5-formylpyridazin-3-yl)carbamate (867 mg 2.32 mmol, 1.00 eq) in THE (10 mL) was then added, and the reaction mixture was stirred at RT for 2 h. The reaction mixture was cooled to 0° C., diluted with H₂O, and extracted with EtOAc. The combined organic layers were dried over Na₂SO₄, filtered, and concentrated under vacuum. The residue was purified by column chromatography (SiO₂, PE:EtOAc=1:0 to 0:1) to afford tert-butyl (E)-(2,4-dimethoxybenzyl)(5-((1-(2,4-dimethoxybenzyl)-2-oxopyrrolidin-3-ylidene)methyl)pyridazin-3-yl)carbamate as a yellow oil (1.87 g, 2.09 mmol, 66% purity (triphenylphosphine oxide impurity), 90% yield). LCMS [M+H]⁺=591.4 m/z.

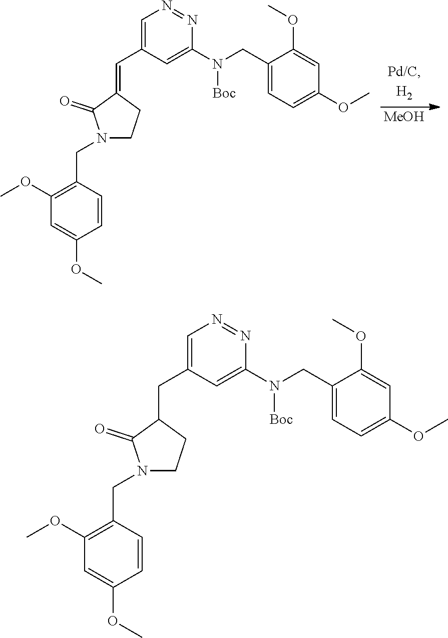

To a solution of tert-butyl (E)-(2,4-dimethoxybenzyl)(5-((1-(2,4-dimethoxybenzyl)-2-oxopyrrolidin-3-ylidene)methyl)pyridazin-3-yl)carbamate (1.87 g, 2.09 mmol, 66% purity, 1.00 eq) in MeOH (98 mL) under nitrogen was added Pd/C (1.11 g, 10% purity). Vacuum was applied followed by replacing with hydrogen three times. The reaction mixture was stirred for 19 h at RT. The hydrogen atmosphere was removed under vacuum and replaced by air. The reaction mixture was filtered through Celite, rinsing with MeOH. The resulting filtrate was concentrated under reduced pressure. The residue was purified by column chromatography (SiO₂, PE:EtOAc=1:0 to 0:1) to afford tert-butyl (2,4-dimethoxybenzyl)(5-((1-(2,4-dimethoxybenzyl)-2-oxopyrrolidin-3-yl)methyl)pyridazin-3-yl)carbamate as a white solid (980 mg, 1.65 mmol, 79% yield). LCMS [M+H]⁺=593.3 m/z.

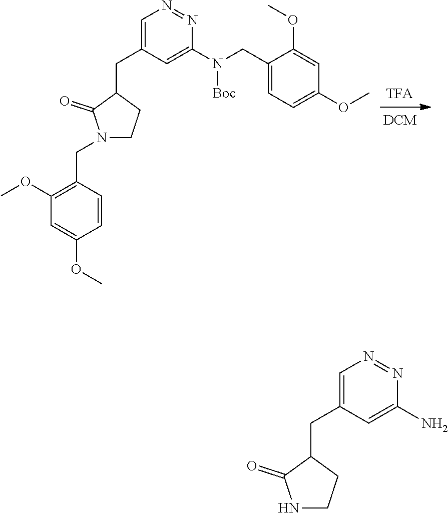

To a solution of tert-butyl (2,4-dimethoxybenzyl)(5-((1-(2,4-dimethoxybenzyl)-2-oxopyrrolidin-3-yl)methyl)pyridazin-3-yl)carbamate (50.4 mg, 85 μmol, 1.0 eq) in DCM (2 mL) under nitrogen at RT was added TFA (390 μL, 5.09 mmol, 60 eq). The reaction mixture was heated at 70° C. for 16 h. The reaction mixture was cooled, diluted with an aqueous solution of NaHCO₃. The aqueous layer was washed with EtOAc. The aqueous layer was lyophilized to obtain crude 3-((6-aminopyridazin-4-yl)methyl)pyrrolidin-2-one as a white solid, which was directly used in the next reaction.

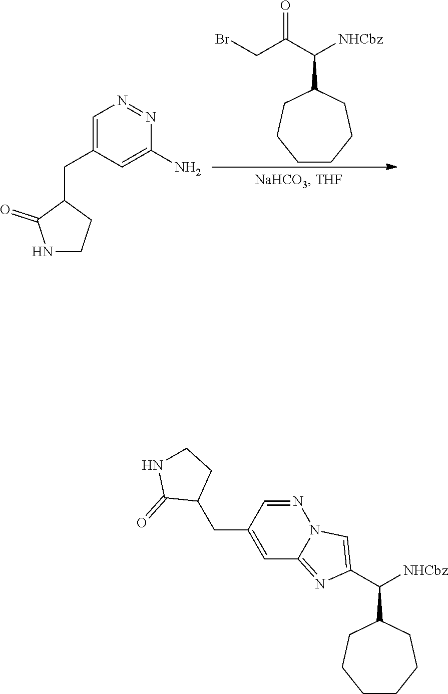 [SMILES: Nc1cc(CC2CCNC2=O)cnn1 ; O=C(N[C@H](C(=O)CBr)C1CCCCCC1)OCc1ccccc1 ; O=C(N[C@H](c1cn2ncc(CC3CCNC3=O)cc2n1)C1CCCCCC1)OCc1ccccc1]

To a solution of 3-((6-aminopyridazin-4-yl)methyl)pyrrolidin-2-one (16.3 mg, 85.0 μmol, 1.00 eq) in THE (1.0 mL) under nitrogen at 0° C. were added benzyl (S)-(3-bromo-1-cycloheptyl-2-oxopropyl)carbamate (Int. 2, 34.4 mg, 90.1 μmol, 1.06 eq) and NaHCO₃ (19.1 mg, 227 μmol, 2.67 eq). The mixture was heated at 70° C. for 16 h. The reaction mixture was concentrated under vacuum and purified by column chromatography (SiO₂, DCM:MeOH=1:0 to 85:15 to afford benzyl ((1 S)-cycloheptyl(7-((2-oxopyrrolidin-3-yl)methyl)imidazo[1,2-b]pyridazin-2-yl)methyl)carbamate as a yellow oil (11 mg, 23.1 μmol, 27% yield over 2 steps). LCMS [M+H]⁺=476.4 m/z.

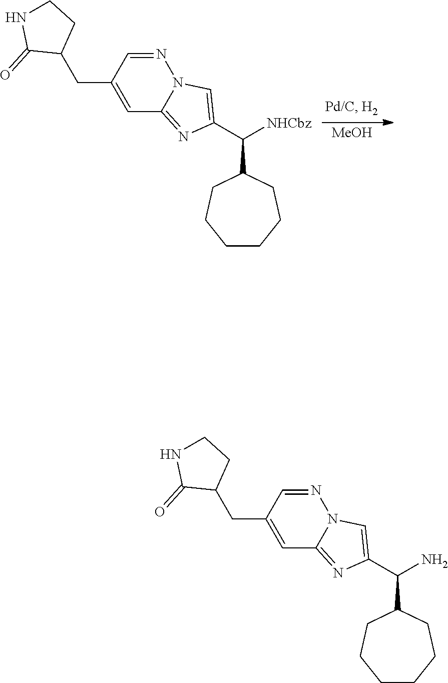 [SMILES: "MeOH" ; O=C(N[C@H](c1cn2ncc(CC3CCNC3=O)cc2n1)C1CCCCCC1)OCc1ccccc1 ; N[C@H](c1cn2ncc(CC3CCNC3=O)cc2n1)C1CCCCCC1]

To a solution of benzyl ((1S)-cycloheptyl(7-((2-oxopyrrolidin-3-yl)methyl)imidazo[1,2-b]pyridazin-2-yl)methyl)carbamate (11 mg, 23.1 μmol) in MeOH (8 mL) under nitrogen was added Pd/C (14.8 mg, 10% purity). The nitrogen atmosphere was removed by vacuum and backfilled with hydrogen three times. The reaction mixture was stirred for 16 h at RT. The hydrogen atmosphere was removed under vacuum and replaced by air. The reaction mixture was then filtered through Celite, rinsing with MeOH. The resulting filtrate was concentrated under reduced pressure to afford crude 3-((2-((S)-amino(cycloheptyl)methyl)imidazo[1,2-b]pyridazin-7-yl)methyl)pyrrolidin-2-one, which was used without further purification in the next reaction. LCMS [M+H]⁺=342.2 m/z.

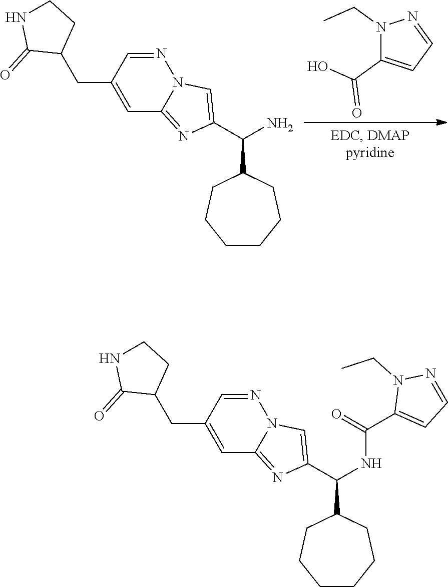

To a solution of 3-((2-((S)-amino(cycloheptyl)methyl)imidazo[1,2-b]pyridazin-7-yl)methyl)pyrrolidin-2-one (7.8 mg, 23.1 μmol) in pyridine (200 μL) were added 1-ethyl-1H-pyrazole-5-carboxylic acid (4.2 mg, 30 umol, 1.3 eq) and EDCI (13.3 mg, 69.3 μmol, 3.0 eq). The reaction mixture was stirred for 1 h at RT. The reaction mixture was twice diluted with heptanes and concentrated under reduced pressure. The crude was then dissolved in DCM and treated with an aqueous solution of NaHCO₃. The aqueous layer was extracted with EtOAc, and the combined organic layers were dried over Na₂SO₄, filtered, and concentrated under vacuum. The residue was purified by column chromatography (SiO₂, DCM:MeOH=1:0 to 85:15 to afford Compound 203, N-((1 S)-cycloheptyl(7-((2-oxopyrrolidin-3-yl)methyl)imidazo[1,2-b]pyridazin-2-yl)methyl)-1-ethyl-1H-pyrazole-5-carboxamide, as a yellow solid (5 mg, 10.8 μmol, 47% yield over 2 steps). LCMS [M+H]⁺=464.3 m/z.

### Example 197: Preparation of Compound 204

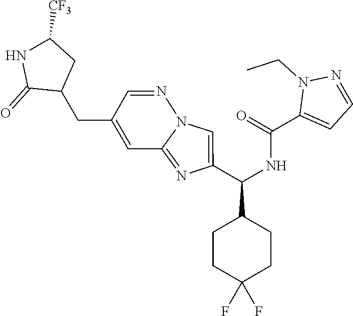

The title compound was prepared according to General Procedure 12, employing tert-butyl (5-(hydroxymethyl)pyridazin-3-yl)carbamate, General Procedure 1, employing tert-butyl (5-(hydroxymethyl)pyridazin-3-yl)carbamate and benzyl (S)-(3-bromo-1-(4,4-difluorocyclohexyl)-2-oxopropyl)carbamate, General Procedure 14, employing benzyl (S)-((4,4-difluorocyclohexyl)(7-(hydroxymethyl)imidazo[1,2-b]pyridazin-2-yl)methyl)carbamate, General Procedure 15, employing benzyl (S)-((7-(chloromethyl)imidazo[1,2-b]pyridazin-2-yl)(4,4-difluorocyclohexyl)methyl) carbamate and 1-(tert-butyl) 3-methyl (5S)-2-oxo-5-(trifluoromethyl)pyrrolidine-1,3-dicarboxylate, General Procedure 9, employing 1-(tert-butyl) 3-methyl (5 S)-3-((2-((S)-(((benzyloxy) carbonyl)amino)(4,4-difluorocyclohexyl)methyl)imidazo[1,2-b]pyridazin-7-yl)methyl)-2-oxo-5-(trifluoromethyl)pyrrolidine-1,3-dicarboxylate, General Procedure 5, employing methyl (5 S)-3-((2-((S)-(((benzyloxy)carbonyl)amino)(4,4-difluorocyclohexyl)methyl)imidazo[1,2-b]pyridazin-7-yl)methyl)-2-oxo-5-(trifluoromethyl)pyrrolidine-3-carboxylate, General Procedure 6, employing (5 S)-3-((2-((S)-(((benzyloxy)carbonyl)amino)(4,4-difluorocyclohexyl)methyl)imidazo[1,2-b]pyridazin-7-yl)methyl)-2-oxo-5-(trifluoromethyl)pyrrolidine-3-carboxylic acid, General Procedure 2, employing benzyl ((1S)-(4,4-difluorocyclohexyl)(7-(((5 S)-2-oxo-5-(trifluoromethyl)pyrrolidin-3-yl)methyl)imidazo[1,2-b]pyridazin-2-yl)methyl)carbamate, General Procedure 4, employing (5 S)-3-((2-((S)-amino(4,4-difluorocyclohexyl)methyl)imidazo[1,2-b]pyridazin-7-yl)methyl)-5-(trifluoromethyl)pyrrolidin-2-one and 1-ethyl-1H-pyrazole-5-carboxylic acid to afford N-((1 S)-(4,4-difluorocyclohexyl)(7-(((5S)-2-oxo-5-(trifluoromethyl)pyrrolidin-3-yl)methyl)imidazo[1,2-b]pyridazin-2-yl)methyl)-1-ethyl-1H-pyrazole-5-carboxamide, which was further purified by chiral prep-SFC (Daicel Chiralcel OD, 250×30 mm, 10 um, eluting with 30% iPrOH (0.1% NH₃—H₂O)) to afford Compound 204 as the first eluting, single stereoisomer (53 mg, 96 umol, 41% yield) and a white solid. LCMS [M+H]⁺=554.2 m/z.

### Example 198: Preparation of Compound 205

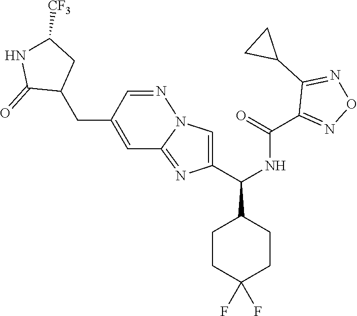

The title compound was prepared according to General Procedure 4, employing (5 S)-3-((2-((S)-amino(4,4-difluorocyclohexyl)methyl)imidazo[1,2-b]pyridazin-7-yl)methyl)-5-(trifluoromethyl)pyrrolidin-2-one and 4-cyclopropyl-1,2,5-oxadiazole-3-carboxylic acid to afford 4-cyclopropyl-N-((1S)-(4,4-difluorocyclohexyl)(7-(((5S)-2-oxo-5-(trifluoromethyl)pyrrolidin-3-yl)methyl)imidazo[1,2-b]pyridazin-2-yl)methyl)-1,2,5-oxadiazole-3-carboxamide, which was further purified by chiral prep-SFC (Daicel Chiralcel OX, 250×30 mm, 10 um, eluting with 30% iPrOH (0.1% NH₃—H₂O)) to afford Compound 205 as the first eluting single stereoisomer (41 mg 72 umol, 62% yield) and a yellow solid. LCMS [M+H]⁺=568.4 m/z.

### Example 199: Preparation of Compound 206

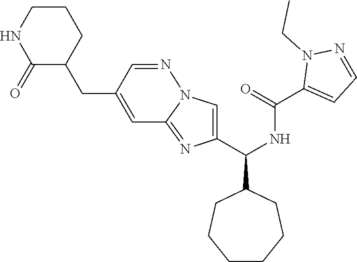

The title compound was prepared according to General Procedure 1, employing (6-aminopyridazin-4-yl)methanol trifluoroacetic acid and benzyl (S)-(3-bromo-1-cycloheptyl-2-oxopropyl)carbamate, General Procedure 14, employing benzyl (S)-(cycloheptyl(7-(hydroxymethyl)imidazo[1,2-b]pyridazin-2-yl)methyl)carbamate, General Procedure 15, employing benzyl (S)-((7-(chloromethyl)imidazo[1,2-b]pyridazin-2-yl)(cycloheptyl)methyl)carbamate and 1-(tert-butyl) 3-methyl 2-oxopiperidine-1,3-dicarboxylate, General Procedure 9, employing 1-(tert-butyl) 3-methyl 3-((2-((S)-(((benzyloxy)carbonyl)amino)(cycloheptyl)methyl)imidazo[1,2-b]pyridazin-7-yl)methyl)-2-oxopiperidine-1,3-dicarboxylate, General Procedure 3, employing methyl 3-((2-((S)-(((benzyloxy)carbonyl)amino)(cycloheptyl)methyl)imidazo[1,2-b]pyridazin-7-yl)methyl)-2-oxopiperidine-3-carboxylatem General Procedure 4, employing methyl 3-((2-((S)-amino(cycloheptyl)methyl)imidazo[1,2-b]pyridazin-7-yl)methyl)-2-oxopiperidine-3-carboxylate and 1-ethyl-1H-pyrazole-5-carboxylic acid, General Procedure 5, employing methyl 3-((2-((S)-cycloheptyl(1-ethyl-1H-pyrazole-5-carboxamido)methyl)imidazo[1,2-b]pyridazin-7-yl)methyl)-2-oxopiperidine-3-carboxylate, and General Procedure 6, employing 3-((2-((S)-cycloheptyl(1-ethyl-1H-pyrazole-5-carboxamido)methyl)imidazo[1,2-b]pyridazin-7-yl)methyl)-2-oxopiperidine-3-carboxylic acid to afford Compound 206 N-((1S)-cycloheptyl(7-((2-oxopiperidin-3-yl)methyl)imidazo[1,2-b]pyridazin-2-yl)methyl)-1-ethyl-1H-pyrazole-5-carboxamide (13 mg, 26 umol, 55% yield) as a mixture of stereoisomers and white solid. LCMS [M+H]⁺=478.4 m/z.

### Example 200: Preparation of Compound 207

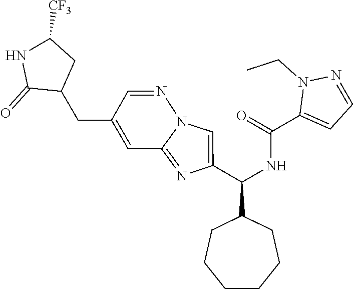

The title compound was prepared according to General Procedure 15, employing benzyl (S)-((7-(chloromethyl)imidazo[1,2-b]pyridazin-2-yl)(cycloheptyl)methyl)carbamate and 1-(tert-butyl) 3-methyl (5 S)-2-oxo-5-(trifluoromethyl)pyrrolidine-1,3-dicarboxylate, General Procedure 9, employing 1-(tert-butyl) 3-methyl (5S)-3-((2-((S)-(((benzyloxy)carbonyl) amino) (cycloheptyl)methyl)imidazo[1,2-b]pyridazin-7-yl)methyl)-2-oxo-5-(trifluoromethyl)pyrrolidine-1,3-dicarboxylate, General Procedure 3, employing methyl (5 S)-3-((2-((S)-(((benzyloxy)carbonyl)amino)(cycloheptyl)methyl)imidazo[1,2-b]pyridazin-7-yl)methyl)-2-oxo-5-(trifluoromethyl)pyrrolidine-3-carboxylate, General Procedure 4, employing methyl (5S)-3-((2-((S)-amino(cycloheptyl)methyl)imidazo[1,2-b]pyridazin-7-yl)methyl)-2-oxo-5-(trifluoromethyl) pyrrolidine-3-carboxylate and 1-ethyl-1H-pyrazole-5-carboxylic acid, General Procedure 5, employing methyl (5S)-3-((2-((S)-cycloheptyl(1-ethyl-1H-pyrazole-5-carboxamido) methyl)imidazo[1,2-b]pyridazin-7-yl)methyl)-2-oxo-5-(trifluoromethyl)pyrrolidine-3-carboxylate, and General Procedure 6, employing (5 S)-3-((2-((S)-cycloheptyl(1-ethyl-1H-pyrazole-5-carboxamido)methyl)imidazo[1,2-b]pyridazin-7-yl)methyl)-2-oxo-5-(trifluoromethyl)pyrrolidine-3-carboxylic acid, to Compound 207 afford N-((l S)-cycloheptyl(7-(((5S)-2-oxo-5-(trifluoromethyl)pyrrolidin-3-yl)methyl)imidazo[1,2-b]pyridazin-2-yl)methyl)-1-ethyl-1H-pyrazole-5-carboxamide (6.2 mg, 12 umol, 16% yield) as a mixture of stereoisomers and white solid. LCMS [M+H]⁺=532.3 m/z.

### Example 201: Preparation of Compound 208

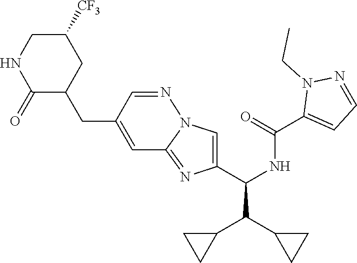

The title compound was prepared according to General Procedure 1, employing methyl (5R)-3-((6-aminopyridazin-4-yl)methyl)-2-oxo-5-(trifluoromethyl)piperidine-3-carboxylate trifluoroacetic acid and benzyl (S)-(4-bromo-1,1-dicyclopropyl-3-oxobutan-2-yl)carbamate, General Procedure 3, employing methyl (5R)-3-((2-((S)-1-(((benzyloxy)carbonyl)amino)-2,2-dicyclopropylethyl)imidazo[1,2-b]pyridazin-7-yl)methyl)-2-oxo-5-(trifluoromethyl)piperidine-3-carboxylate, General Procedure 4, employing methyl (5R)-3-((2-((S)-1-amino-2,2-dicyclopropylethyl)imidazo[1,2-b]pyridazin-7-yl)methyl)-2-oxo-5-(trifluoromethyl)piperidine-3-carboxylate and 1-ethyl-1H-pyrazole-5-carboxylic acid, General Procedure 5, employing methyl (5R)-3-((2-((S)-2,2-dicyclopropyl-1-(1-ethyl-1H-pyrazole-5-carboxamido)ethyl)imidazo[1,2-b]pyridazin-7-yl)methyl)-2-oxo-5-(trifluoromethyl)piperidine-3-carboxylate, and General Procedure 6, employing methyl (5R)-3-((2-((S)-2,2-dicyclopropyl-1-(1-ethyl-1H-pyrazole-5-carboxamido)ethyl)imidazo[1,2-b]pyridazin-7-yl)methyl)-2-oxo-5-(trifluoromethyl)piperidine-3-carboxylate, to afford N-((1S)-2,2-dicyclopropyl-1-(7-(((5R)-2-oxo-5-(trifluoromethyl)piperidin-3-yl)methyl)imidazo[1,2-b]pyridazin-2-yl)ethyl)-1-ethyl-1H-pyrazole-5-carboxamide, which was further purified by chiral prep-SFC (Daicel Chiralcel AS, 250×30 mm, 10 um, eluting with 35% EtOH (0.1% NH₃—H₂O)) to afford Compound 208 as the first eluting, single stereoisomer (12 mg, 20 umol, 27% yield) and a yellow solid. LCMS [M+H]+=544.5 m/z.

### Example 202: Preparation of Compound 209

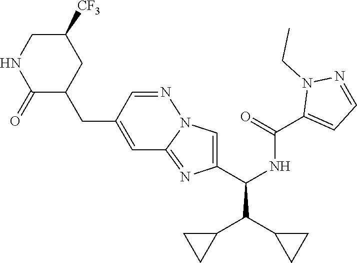

The title compound was prepared according to General Procedure 8, employing tert-butyl (5-(chloromethyl)pyridazin-3-yl)carbamate and 1-(tert-butyl) 3-methyl (5S)-2-oxo-5-(trifluoromethyl)piperidine-1,3-dicarboxylate, General Procedure 12, employing 1-(tert-butyl) 3-methyl (5S)-3-((6-((tert-butoxycarbonyl)amino)pyridazin-4-yl)methyl)-2-oxo-5-(trifluoromethyl)piperidine-1,3-dicarboxylate, General Procedure 1, employing methyl (5S)-3-((6-aminopyridazin-4-yl)methyl)-2-oxo-5-(trifluoromethyl)piperidine-3-carboxylate and benzyl (S)-(4-bromo-1,1-dicyclopropyl-3-oxobutan-2-yl)carbamate, General Procedure 2, employing methyl (5S)-3-((2-((S)-1-(((benzyloxy)carbonyl)amino)-2,2-dicyclopropylethyl)imidazo[1,2-b]pyridazin-7-yl)methyl)-2-oxo-5-(trifluoromethyl)piperidine-3-carboxylate, General Procedure 4, employing methyl (5 S)-3-((2-((S)-1-amino-2,2-dicyclopropylethyl)imidazo[1,2-b]pyridazin-7-yl)methyl)-2-oxo-5-(trifluoromethyl)piperidine-3-carboxylate and 1-ethyl-1H-pyrazole-5-carboxylic acid, and General Procedure 5, employing 1-ethyl-1H-pyrazole-5-carboxylic acid, and General Procedure 6, employing (5S)-3-((2-((S)-2,2-dicyclopropyl-1-(1-ethyl-1H-pyrazole-5-carboxamido)ethyl)imidazo[1,2-b]pyridazin-7-yl)methyl)-2-oxo-5-(trifluoromethyl)piperidine-3-carboxylic acid, to afford N-((1S)-2,2-dicyclopropyl-1-(7-(((5S)-2-oxo-5-(trifluoromethyl)piperidin-3-yl)methyl)imidazo[1,2-b]pyridazin-2-yl)ethyl)-1-ethyl-1H-pyrazole-5-carboxamide, which was further purified by chiral prep-SFC (Daicel Chiralcel AS, 250×30 mm, 10 um, eluting with 35% iPrOH (0.1% NH₃—H₂O)) to afford Compound 209 as the second eluting, single stereoisomer (5.3 mg, 8.7 umol, 13% yield) and a yellow solid. LCMS [M+H]⁺=544.3 m/z.

### Example 203: Preparation of Compound 210 and Compound 211

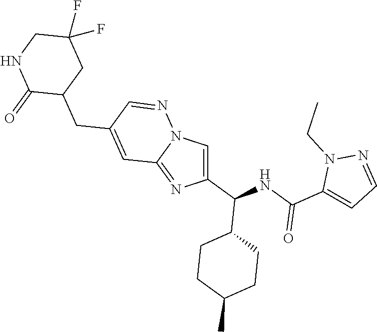 [SMILES: CCn1nccc1C(=O)N[C@H](c1cn2ncc(CC3CC(F)(F)CNC3=O)cc2n1)[C@H]1CC[C@H](C)CC1]

The title compound was prepared according to General Procedure 1, employing (6-aminopyridazin-4-yl)methanol trifluoroacetic acid and benzyl ((S)-3-bromo-1-((1r,4S)-4-methylcyclohexyl)-2-oxopropyl)carbamate, General Procedure 3, employing benzyl ((S)-(7-(hydroxymethyl)imidazo[1,2-b]pyridazin-2-yl)((1r,4S)-4-methylcyclohexyl)methyl)carbamate, General Procedure 4, employing (2-((S)-amino((1r,4S)-4-methylcyclohexyl)methyl)imidazo[1,2-b]pyridazin-7-yl)methanol and 1-ethyl-1H-pyrazole-5-carboxylic acid, General Procedure 14, employing 1-ethyl-N—((S)-(7-(hydroxymethyl)imidazo[1,2-b]pyridazin-2-yl)((1r,4S)-4-methylcyclohexyl)methyl)-1H-pyrazole-5-carboxamide, General Procedure 15, employing N—((S)-(7-(chloromethyl)imidazo[1,2-b]pyridazin-2-yl)((1r,4S)-4-methylcyclohexyl)methyl)-1-ethyl-1H-pyrazole-5-carboxamide and 1-(tert-butyl) 3-methyl 5,5-difluoro-2-oxopiperidine-1,3-dicarboxylate, General Procedure 9, employing 1-(tert-butyl) 3-methyl 3-((2-((S)-(1-ethyl-1H-pyrazole-5-carboxamido)((1r,4S)-4-methylcyclohexyl)methyl)imidazo[1,2-b]pyridazin-7-yl)methyl)-5,5-difluoro-2-oxopiperidine-1,3-dicarboxylate, General Procedure 5, employing methyl 3-((2-((S)-(1-ethyl-1H-pyrazole-5-carboxamido)((1r,4S)-4-methylcyclohexyl)methyl)imidazo[1,2-b]pyridazin-7-yl)methyl)-5,5-difluoro-2-oxopiperidine-3-carboxylate, and General Procedure 6, employing 3-((2-((S)-(1-ethyl-1H-pyrazole-5-carboxamido)((1r,4S)-4-methylcyclohexyl)methyl)imidazo[1,2-b]pyridazin-7-yl)methyl)-5,5-difluoro-2-oxopiperidine-3-carboxylic acid, to afford N-((1S)-(7-((5,5-difluoro-2-oxopiperidin-3-yl)methyl)imidazo[1,2-b]pyridazin-2-yl)((1r,4S)-4-methylcyclohexyl)methyl)-1-ethyl-1H-pyrazole-5-carboxamide, which was further purified by chiral prep-SFC (Daicel Chiralpack AD, 250×30 mm, 10 um, eluting with 50% EtOH (0.1% NH₃—H₂O)) to afford Compound 210 as the first eluting, single stereoisomer (23 mg, 45 umol, 38% yield) and a white solid. LCMS [M+H]+=514.3 m/z. Compound 211 was isolated as the second eluting, single stereoisomer (23 mg, 43 umol, 37% yield) and a white solid. LCMS [M+H]⁺=514.3 m/z.

### Example 204: Preparation of Compound 212

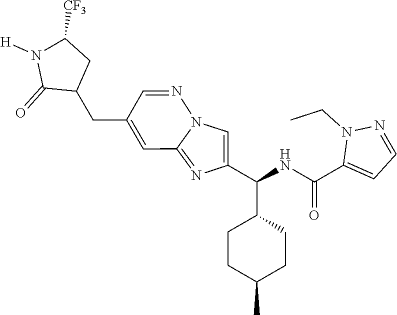

The title compound was prepared according to General Procedure 15, employing N—((S)-(7-(chloromethyl)imidazo[1,2-b]pyridazin-2-yl)((1r,4S)-4-methylcyclohexyl)methyl)-1-ethyl-1H-pyrazole-5-carboxamide and 1-(tert-butyl) 3-methyl (5S)-2-oxo-5-(trifluoromethyl)pyrrolidine-1,3-dicarboxylate, General Procedure 9, employing methyl (5S)-3-((2-((S)-(1-ethyl-1H-pyrazole-5-carboxamido)((1r,4S)-4-methylcyclohexyl)methyl)imidazo[1,2-b]pyridazin-7-yl)methyl)-2-oxo-5-(trifluoromethyl)pyrrolidine-3-carboxylate, General Procedure 5, employing 1-(tert-butyl) 3-methyl (5S)-3-((2-((S)-(1-ethyl-1H-pyrazole-5-carboxamido)((1r,4S)-4-methylcyclohexyl)methyl)imidazo[1,2-b]pyridazin-7-yl)methyl)-2-oxo-5-(trifluoromethyl)pyrrolidine-1,3-dicarboxylate, and General Procedure 6, employing (5S)-3-((2-((S)-(1-ethyl-1H-pyrazole-5-carboxamido)((1r,4S)-4-methylcyclohexyl)methyl)imidazo[1,2-b]pyridazin-7-yl)methyl)-2-oxo-5-(trifluoromethyl) pyrrolidine-3-carboxylic acid, to afford 1-ethyl-N-((1 S)-((1r,4S)-4-methylcyclohexyl)(7-(((5S)-2-oxo-5-(trifluoromethyl)pyrrolidin-3-yl)methyl)imidazo[1,2-b]pyridazin-2-yl)methyl)-1H-pyrazole-5-carboxamide, which was further purified by chiral prep-SFC (Daicel Chiralcel OJ-H, 250×30 mm, 5 um, eluting with 20% EtOH (0.1% NH₃—H₂O)) to afford Compound 212 as the second eluting, single stereoisomer (45 mg 84 umol, 64% yield) and a white solid. LCMS [M+H]⁺=532.3 m/z.

### Example 205: Preparation of Compound 213 and Compound 214

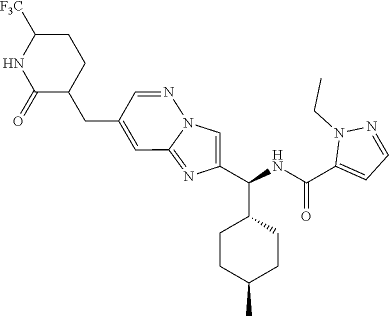

The title compound was prepared according to General Procedure 15, employing N—((S)-(7-(chloromethyl)imidazo[1,2-b]pyridazin-2-yl)((1r,4S)-4-methylcyclohexyl)methyl)-1-ethyl-1H-pyrazole-5-carboxamide and 1-(tert-butyl) 3-methyl 2-oxo-6-(trifluoromethyl)piperidine-1,3-dicarboxylate, General Procedure 9, employing 1-(tert-butyl) 3-methyl 3-((2-((S)-(1-ethyl-1H-pyrazole-5-carboxamido)((1r,4S)-4-methylcyclohexyl)methyl)imidazo[1,2-b]pyridazin-7-yl)methyl)-2-oxo-6-(trifluoromethyl)piperidine-1,3-dicarboxylate, General Procedure 5, employing methyl 3-((2-((S)-(1-ethyl-1H-pyrazole-5-carboxamido)((1r,4S)-4-methylcyclohexyl)methyl)imidazo[1,2-b]pyridazin-7-yl)methyl)-2-oxo-6-(trifluoromethyl) piperidine-3-carboxylate, General Procedure 6, employing 3-((2-((S)-(1-ethyl-1H-pyrazole-5-carboxamido)((1r,4S)-4-methylcyclohexyl)methyl)imidazo[1,2-b]pyridazin-7-yl)methyl)-2-oxo-6-(trifluoromethyl)piperidine-3-carboxylic acid, to afford 1-ethyl-N-((1S)-((1r,4S)-4-methylcyclohexyl)(7-((2-oxo-6-(trifluoromethyl)piperidin-3-yl)methyl)imidazo[1,2-b]pyridazin-2-yl)methyl)-1H-pyrazole-5-carboxamide, which was further purified by chiral prep-SFC (Daicel Chiralpak AS, 250×30 mm, 5 um, eluting with 20% EtOH (0.1% NH₃—H₂O)) to afford Compound 213 as the first eluting, single stereoisomer (37 mg, 65 umol, 15% yield) and a white solid. LCMS [M+H]⁺=546.2 m/z. Compound 214 was isolated as the third eluting, single stereoisomer (61 mg, 110 umol, 25% yield) and a white solid. LCMS [M+H]⁺=546.2 m/z.

### Example 206: Preparation of Compound 215

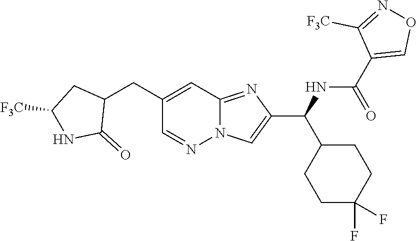 [SMILES: O=C(N[C@H](c1cn2ncc(CC3C[C@@H](C(F)(F)F)NC3=O)cc2n1)C1CCC(F)(F)CC1)c1conc1C(F)(F)F]

The title compound was prepared according to General Procedure 4, employing (5S)-3-((6-((S)-amino(4,4-difluorocyclohexyl)methyl)pyrrolo[1,2-b]pyridazin-3-yl)methyl)-5-(trifluoromethyl)pyrrolidin-2-one and 3-(trifluoromethyl)isoxazole-4-carboxylic acid to afford N-((1S)-(4,4-difluorocyclohexyl)(7-(((5 S)-2-oxo-5-(trifluoromethyl)pyrrolidin-3-yl)methyl)imidazo[1,2-b]pyridazin-2-yl)methyl)-3-(trifluoromethyl)isoxazole-4-carboxamide, which was further purified by chiral prep-SFC (Daicel Chiralcel OD, 250×30 mm, 10 um, eluting with 35% iPrOH to afford Compound 215 as the second eluting, single stereoisomer (8 mg, 10 umol, 6% yield) and a white solid. LCMS [M+H]⁺=595.3 m/z.

### Example 207: Preparation of Compound 216

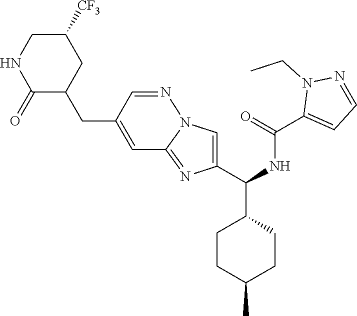

The title compound was prepared according to General Procedure 15, employing N—((S)-(7-(chloromethyl)imidazo[1,2-b]pyridazin-2-yl)((1r,4S)-4-methylcyclohexyl)methyl)-1-ethyl-1H-pyrazole-5-carboxamide and 1-(tert-butyl) 3-methyl (5R)-3-((2-((S)-(1-ethyl-1H-pyrazole-5-carboxamido)((1r,4S)-4-methylcyclohexyl)methyl)imidazo[1,2-b]pyridazin-7-yl)methyl)-2-oxo-5-(trifluoromethyl)piperidine-1,3-dicarboxylate, General Procedure 9, employing 1-(tert-butyl)3-methyl (5R)-3-((2-((S)-(1-ethyl-1H-pyrazole-5-carboxamido)((1r,4S)-4-methylcyclohexyl) methyl)imidazo[1,2-b]pyridazin-7-yl)methyl)-2-oxo-5-(trifluoromethyl)piperidine-1,3-dicarboxylate, General Procedure 5, employing methyl (5R)-3-((2-((S)-(1-ethyl-1H-pyrazole-5-carboxamido)((1r,4S)-4-methylcyclohexyl)methyl)imidazo[1,2-b]pyridazin-7-yl)methyl)-2-oxo-5-(trifluoromethyl)piperidine-3-carboxylate, and General Procedure 6, (5R)-3-((2-((S)-(1-ethyl-1H-pyrazole-5-carboxamido)((1r,4S)-4-methylcyclohexyl)methyl)imidazo[1,2-b]pyridazin-7-yl)methyl)-2-oxo-5-(trifluoromethyl)piperidine-3-carboxylic acid, to afford 1-ethyl-N-((1S)-((1r,4S)-4-methylcyclohexyl)(7-(((5R)-2-oxo-5-(trifluoromethyl)piperidin-3-yl)methyl)imidazo[1,2-b]pyridazin-2-yl)methyl)-1H-pyrazole-5-carboxamide, which was further purified by chiral prep-SFC (Daicel Chiralpak AS, 250×30 mm, 10 um, eluting with 30% MeOH to afford Compound 216 as the first eluting, single stereoisomer (60 mg, 110 umol, 60% yield) and a white solid. LCMS [M+H]⁺=546.2 m/z.

### Example 208: Preparation of Compound 217 and Compound 218

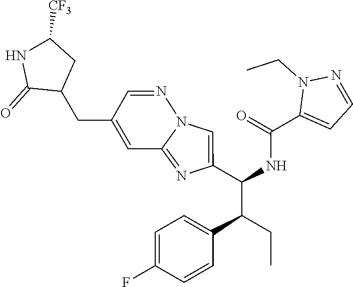

The title compound was prepared according to General Procedure 1, employing (6-aminopyridazin-4-yl)methanol and benzyl ((3S,4S)-1-bromo-4-(4-fluorophenyl)-2-oxohexan-3-yl)carbamate, General Procedure 14, employing benzyl ((1S,2S)-2-(4-fluorophenyl)-1-(7-(hydroxymethyl)imidazo[1,2-b]pyridazin-2-yl)butyl)carbamate, General Procedure 15, employing 1-(tert-butyl) 3-methyl (5S)-2-oxo-5-(trifluoromethyl)pyrrolidine-1,3-dicarboxylate and benzyl ((1S,2S)-1-(7-(chloromethyl)imidazo[1,2-b]pyridazin-2-yl)-2-(4-fluorophenyl)butyl)carbamate, General Procedure 9, employing 1-(tert-butyl) 3-methyl (5 S)-3-((2-((1 S,2S)-1-(((benzyloxy)carbonyl)amino)-2-(4-fluorophenyl)butyl)imidazo[1,2-b]pyridazin-7-yl)methyl)-2-oxo-5-(trifluoromethyl)pyrrolidine-1,3-dicarboxylate, General Procedure 5, employing methyl (5S)-3-((2-((1S,2S)-1-(((benzyloxy)carbonyl)amino)-2-(4-fluorophenyl)butyl)imidazo[1,2-b]pyridazin-7-yl)methyl)-2-oxo-5-(trifluoromethyl)pyrrolidine-3-carboxylate, General Procedure 6, employing (5S)-3-((2-((1S,2S)-1-(((benzyloxy)carbonyl)amino)-2-(4-fluorophenyl)butyl)imidazo[1,2-b]pyridazin-7-yl)methyl)-2-oxo-5-(trifluoromethyl)pyrrolidine-3-carboxylic acid, General Procedure 2, employing benzyl ((1S,2S)-2-(4-fluorophenyl)-1-(7-(((5S)-2-oxo-5-(trifluoromethyl)pyrrolidin-3-yl)methyl)imidazo[1,2-b]pyridazin-2-yl)butyl)carbamate, and General Procedure 4, employing (5S)-3-((2-((1 S,2S)-1-amino-2-(4-fluorophenyl)butyl)imidazo[1,2-b]pyridazin-7-yl)methyl)-5-(trifluoromethyl)pyrrolidin-2-one and 1-ethyl-1H-pyrazole-5-carboxylic acid, to afford 1-ethyl-N-((1 S,2S)-2-(4-fluorophenyl)-1-(7-(((5S)-2-oxo-5-(trifluoromethyl)pyrrolidin-3-yl)methyl)imidazo[1,2-b]pyridazin-2-yl)butyl)-1H-pyrazole-5-carboxamide, which was further purified by chiral prep-SFC (Daicel Chiralpak OJ-H, 250×30 mm, 5 um, eluting with 25% EtOH (0.1% NH₃—H₂O) to afford the title Compound 217 the first eluting, single stereoisomer (4 mg 6 umol, 5% yield) and a white solid. LCMS [M+H]⁺=572.3 m/z. Compound 218 was isolated as the second eluting, single stereoisomer (20 mg, 35 umol, 29% yield) as a white solid. LCMS [M+H]⁺=572.4 m/z.

### Example 209: Preparation of Compound 219

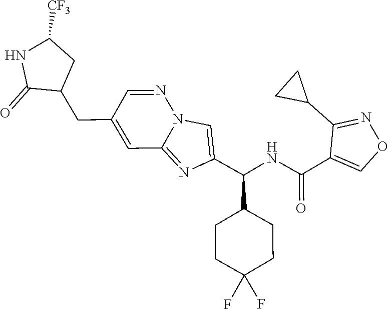 [SMILES: O=C(N[C@H](c1cn2ncc(CC3C[C@@H](C(F)(F)F)NC3=O)cc2n1)C1CCC(F)(F)CC1)c1conc1C1CC1]

The title compound was prepared according to General Procedure 4, employing (5 S)-3-((2-((S)-amino(4,4-difluorocyclohexyl)methyl)imidazo[1,2-b]pyridazin-7-yl)methyl)-5-(trifluoromethyl)pyrrolidin-2-one and 3-cyclopropylisoxazole-4-carboxylic acid to afford 3-cyclopropyl-N-((1S)-(4,4-difluorocyclohexyl)(7-(((5S)-2-oxo-5-(trifluoromethyl)pyrrolidin-3-yl)methyl)imidazo[1,2-b]pyridazin-2-yl)methyl)isoxazole-4-carboxamide, which was further purified by chiral prep-SFC (Daicel Chiralcel OD, 250×30 mm, 10 um, eluting with 45% MeOH (0.1% NH₃—H₂O) to afford Compound 219 as the first eluting, single stereoisomer (116 mg 202 umol, 77% yield) and a white solid. LCMS [M+H]⁺=567.4 m/z.

### Example 210: IL-17A/A HEK-Blue Cell Assay

The HEK-Blue IL-17A reporter cell line (Fisher #NC1408637) was used for cell-based IL-17A/A inhibition assays. Cells were grown and prepared for assays according to the manufacturer's instructions. This cell line consists of HEK 293 cells that were designed to expressed IL-17RA, IL-17RC, and the ActI adapter molecule, the combination of which, when stimulated by IL-17A/A activates a NFκB promoter and drives expression of a recombinant Secreted Alkaline Phosphatase (SEAP) gene protein. Media from the cells is then added to a development reagent (Quanti-Blue Substrate, Fisher #NC9711613), and read at A₆₃₀.

Compounds were titrated in DMSO, with a top final compound concentration of 10 uM, 1 uM, or 0.3 uM, and added to the cells immediately before adding IL-17A/A (Genscript #Z03228). The cells, compound, and IL-17A/A were then incubated for 20 hours before media was removed for SEAP analysis. The resulting inhibition curve was then analyzed using Graphpad Prism 7.0, and IC₅₀ values were determined using a 4-parameter nonlinear fit. DMSO was added to a universal final concentration of 0.1% to optimize background.

Table 1 includes IC₅₀ values for IL-17A/A inhibition of selected compounds; with compounds having a IC₅₀ of A<100 nM; B 100-1000 nM; and C>1000 nM.

TABLE 1
IL-17 A/A Inhibition Data for selected compounds

| Compound | IL 17 |
|---|---|
| No. | IC₅₀ (nM) |
| 1 | C |
| 2 | C |
| 3 | A |
| 4 | B |
| 5 | A |
| 6 | C |
| 7 | B |
| 8 | B |
| 9 | A |
| 10 | B |
| 11 | C |
| 12 | A |
| 13 | B |
| 14 | B |
| 15 | C |
| 16 | B |
| 17 | B |
| 18 | C |
| 19 | B |
| 20 | B |
| 21 | C |
| 22 | B |
| 23 | C |
| 24 | C |
| 25 | B |
| 26 | B |
| 27 | B |
| 28 | B |
| 29 | C |
| 30 | B |
| 31 | B |
| 32 | B |
| 33 | B |
| 34 | C |
| 35 | B |
| 36 | C |
| 37 | C |
| 38 | C |
| 39 | B |
| 40 | C |
| 41 | C |
| 42 | C |
| 43 | C |
| 44 | B |
| 45 | C |
| 46 | C |
| 47 | C |
| 48 | B |
| 49 | B |
| 50 | C |
| 51 | B |
| 52 | C |
| 53 | B |
| 54 | B |
| 55 | B |
| 56 | C |
| 57 | C |
| 58 | C |
| 59 | B |
| 60 | B |
| 61 | B |
| 62 | B |
| 63 | B |
| 64 | B |
| 65 | B |
| 66 | B |
| 67 | B |
| 68 | C |
| 69 | B |
| 70 | B |
| 71 | B |
| 72 | B |
| 73 | B |
| 74 | C |
| 75 | C |
| 76 | B |
| 77 | C |
| 78 | B |
| 79 | C |
| 80 | B |
| 81 | B |
| 82 | C |
| 83 | B |
| 84 | B |
| 85 | B |
| 86 | B |
| 87 | A |
| 88 | A |
| 89 | A |
| 90 | B |
| 91 | A |
| 92 | A |
| 93 | B |
| 94 | A |
| 95 | B |
| 96 | B |
| 97 | B |
| 98 | A |
| 99 | B |
| 100 | B |
| 101 | B |
| 102 | A |
| 103 | B |
| 104 | A |
| 105 | B |
| 106 | B |
| 107 | B |
| 108 | B |
| 109 | B |
| 110 | B |
| 111 | A |
| 112 | B |
| 113 | B |
| 114 | B |
| 115 | B |
| 116 | B |
| 117 | A |
| 118 | B |
| 119 | B |
| 120 | B |
| 121 | B |
| 122 | B |
| 123 | B |
| 124 | B |
| 125 | B |
| 126 | A |
| 127 | A |
| 128 | B |
| 129 | C |
| 130 | C |
| 131 | B |
| 132 | C |
| 133 | B |
| 134 | B |
| 135 | C |
| 136 | B |
| 137 | B |
| 138 | B |
| 139 | C |
| 140 | B |
| 141 | A |
| 142 | A |
| 143 | A |
| 144 | A |
| 145 | A |
| 146 | A |
| 147 | B |
| 148 | A |
| 149 | A |
| 150 | A |
| 151 | A |
| 152 | B |
| 153 | B |
| 154 | A |
| 155 | A |
| 156 | B |
| 157 | A |
| 158 | B |
| 159 | A |
| 160 | A |
| 161 | A |
| 162 | B |
| 163 | B |
| 164 | C |
| 165 | B |
| 166 | C |
| 167 | B |
| 168 | A |
| 169 | B |
| 170 | C |
| 171 | B |
| 172 | C |
| 173 | B |
| 174 | C |
| 175 | B |
| 176 | A |
| 177 | A |
| 178 | C |
| 179 | C |
| 180 | A |
| 181 | A |
| 182 | B |
| 183 | A |
| 184 | B |
| 185 | A |
| 186 | A |
| 187 | B |
| 188 | B |
| 189 | B |
| 190 | C |
| 191 | B |
| 192 | C |
| 193 | C |
| 194 | C |
| 195 | C |
| 201 | C |
| 202 | B |
| 203 | C |
| 204 | A |
| 205 | A |
| 206 | C |
| 207 | A |
| 208 | B |
| 209 | C |
| 210 | C |
| 211 | B |
| 212 | A |
| 213 | C |
| 214 | B |
| 215 | A |
| 216 | B |
| 217 | C |
| 218 | A |
| 219 | A |

We claim:

1. A compound represented by structure of Formula (I): 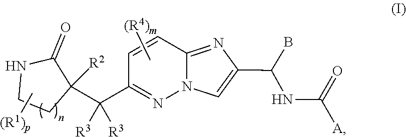
or a pharmaceutically acceptable salt thereof wherein:
A is selected from 5- to 6-membered heteroaryl and C₃₋₆ carbocycle, any of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —(R¹¹)C(O)R¹¹, —C(O)OR¹¹, —OC(O)R¹¹, —NO₂, ═O, ═N(R¹¹), and —CN; and
C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, OC(O)R^{11A}, —NO₂, ═O, ═N(R^{11A}), and —CN;
B is selected from —C(H)(R⁵)₂ and C₃₋₁₀ carbocycle, wherein each C₃₋₁₀ carbocycle is optionally substituted with one or more substituents independently selected from:
halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁵ is independently selected at each occurrence from (i), (ii), and (iii):
(i) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
(ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³,
—N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
(iii) C₃₋₁₀ carbocycle optionally substituted with one or more substituents independently selected from:
halogen, —OR¹³, —SR¹³, —N(R¹³)₂, —C(O)R¹³, —C(O)N(R¹³)₂, —N(R¹³)C(O)R¹³,
—N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
C₁₋₁₀ alkyl optionally substituted with one or more substituents independently selected from halogen, —OR¹³, —SR¹³, —N(R¹³)₂, —C(O)R¹³, —C(O)N(R¹³)₂,
—N(R¹³)C(O)R¹³, —C(O)OR¹³, —OC(O)R¹³, —S(O)R¹³, —S(O)₂R¹³, —NO₂, ═O, ═S, ═N(R¹³), and —CN;
each R¹ is independently selected at each occurrence from:
halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN;
C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN; and
C₃₋₁₀ carbocycle optionally substituted with one or more substituents independently selected from halogen, —OR^{14A}, —SR^{14A}, —N(R^{14A})₂, —C(O)R^{14A}, —C(O)N(R^{14A})₂, —N(R^{14A})C(O)R^{14A}, —C(O)OR^{14A}, —OC(O)R^{14A}, —S(O)R^{14A}, —S(O)₂R^{14A}, —NO₂, ═O, ═S, ═N(R^{14A}), —CN; and C₁₋₁₀ alkyl optionally substituted with one or more substituents independently selected from halogen, —OR^{14A}, —SR^{14A}, —N(R^{14A})₂, —C(O)R^{14A}, —C(O)N(R^{14A})₂, —N(R^{14A})C(O)R^{14A}, —C(O)OR^{14A}, —OC(O)R^{14A}, —S(O)R^{14A}, —S(O)₂R^{14A}, —NO₂, ═O, ═S, ═N(R^{14A}), and —CN; or
two R¹ substituents may come together to form a C₃₋₆ carbocycle, wherein the C₃₋₆ carbocycle is optionally substituted with one or more substituents independently selected from:
halogen, —OR^{18A}, —N(R^{18A})₂, —C(O)R^{18A}, —C(O)N(R^{18A})₂, —NO₂, ═O, ═S, ═N(R^{18A}), —CN; and C₁₋₁₀ alkyl optionally substituted with one or more substituents independently selected from halogen, —OR^{18A}, —N(R^{18A})₂, —C(O)R^{18A}, —C(O)N(R^{18A})₂, —NO₂, ═O, ═S, ═N(R^{18A}), and —CN;
R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN; and C₁₋₆ alkyl optionally substituted with one or more substituents independently selected from halogen, —OR¹⁵, —SR^{IS}, —N(R¹⁵)₂, —C(O)R¹⁵, —C(O)N(R¹⁵)₂, —N(R¹⁵)C(O)R¹⁵, —C(O)OR¹⁵,
—OC(O)R¹⁵, —S(O)R¹⁵, —S(O)₂R¹⁵, —NO₂, ═O, ═S, ═N(R¹⁵), and —CN;
each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂,
—N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN;
each R⁴ is independently selected at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R^{17A})C(O)R^{17A}, —N(R¹⁷)S(O)₂R¹⁷, —C(O)OR¹⁷, —OC(O)R¹⁷, —S(O)R¹⁷, —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, and —CN;
R¹¹, R^{11A}, R¹², R¹³, R¹⁴, R^{14A}, R¹⁵, R¹⁶, R¹⁷, R^{17A}, and R^{18A}, are each independently selected at each occurrence from:
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2;
m is selected from 0, 1 and 2; and
p is selected from 0, 1, 2, 3, 4, 5, and 6.

2. The compound or salt of claim 1, wherein A is selected from 5- to 6-membered heteroaryl and C₃₋₆ carbocycle, any of which is optionally substituted with one or more substituents independently selected from C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

3. The compound or salt of claim 2, wherein A is 5- to 6-membered heteroaryl optionally substituted with one or more substituents independently selected from C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

4. The compound or salt of claim 3, wherein A is 5-membered heteroaryl optionally substituted with C₃₋₁₀ carbocycle optionally substituted with one or more substituents independently selected from halogen, —OR^{11A}, —N(R^{11A})₂, —C(O)R^{11A}, —NO₂, —CN; and C₁₋₁₀ alkyl optionally substituted with one or more substituents selected from: halogen, —OR¹¹A, —N(R^{11A})₂, —C(O)R^{11A}, —NO₂, and —CN.

5. The compound or salt of claim 4, wherein A is selected from pyrazolyl, imidazolyl, oxazolyl, isoxazolyl, oxadiazolyl, optionally substituted with C₃₋₆ cycloalkyl optionally substituted with one or more substituents independently selected from halogen, —OR¹¹A, —N(R^{11A})₂, —C(O)R^{11A}, —NO₂, —CN, C₁₋₆ alkyl, and C₁₋₆ haloalkyl.

6. The compound or salt of claim 5, wherein A is selected from:

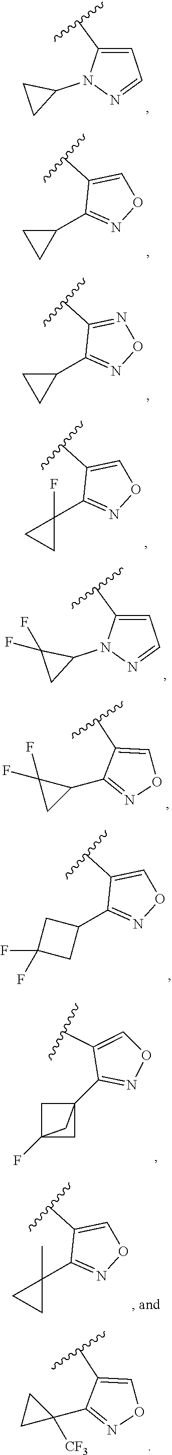

7. The compound or salt of claim 1, wherein R¹ is C₃₋₁₀ carbocycle optionally substituted with one or more substituents independently selected from halogen, —OR^{14A}, —SR^{14A}, —N(R^{14A})₂, —C(O)R^{14A}, —C(O)N(R^{14A})₂, —N(R^{14A})C(O)R^{14A}, —C(O)OR^{14A}, —OC(O)R^{14A}, —S(O)R^{14A}, —S(O)₂R^{14A}, —NO₂, ═O, ═S, ═N(R^{14A}), —CN; and C₁₋₁₀ alkyl optionally substituted with one or more substituents independently selected from halogen, —OR^{14A}, SR^{14A}, —N(R^{14A})₂, —C(O)R^{14A}, —C(O)N(R^{14A})₂, —N(R^{14A})C(O)R^{14A}, —C(O)OR^{14A}, —OC(O)R^{14A}, —S(O)R^{14A}, —S(O)₂R^{14A}, —NO₂, ═O, ═S, ═N(R^{14A}), and —CN.

8. The compound or salt of claim 7, wherein R¹ is C₃₋₆ carbocycle optionally substituted with one or more substituents independently selected from halogen, —OR^{14A}, —N(R^{14A})₂, —C(O)R^{14A}, —NO₂, —CN; and C₁₋₆ alkyl optionally substituted with one or more substituents independently selected from halogen, —OR^{14A}, —N(R^{14A})₂, —C(O)R^{14A}, —NO₂, and —CN.

9. The compound or salt of claim 8, wherein R¹ is C₃₋₆ cycloalkyl optionally substituted with one or more substituents independently selected from halogen, —OR^{14A}, —N(R^{14A})₂, —C(O)R^{14A}, —NO₂, —CN, C₁₋₆ alkyl, and C₁₋₆ haloalkyl.

10. The compound or salt of claim 9, wherein R¹ is

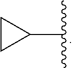 [SMILES: *C1CC1]

11. The compound or salt of claim 1, wherein two R¹ substituents may come together to form a C₃₋₆ carbocycle, wherein the C₃₋₆ carbocycle is optionally substituted with one or more substituents independently selected from: halogen, —OR^{18A}, —N(R^{18A})₂, —C(O)R^{18A}, —C(O)N(R^{18A})₂, —NO₂, ═O, ═S, ═N(R^{18A}), —CN; and C₁₋₁₀ alkyl optionally substituted with one or more substituents independently selected from halogen, —OR^{18A}, —N(R^{18A})₂, —C(O)R^{18A}, —C(O)N(R^{18A})₂, —NO₂, ═O, ═S, ═N(R^{18A}), and —CN.

12. The compound or salt of claim 11, wherein two R¹ substituents may come together to form a C₃₋₆ cycloalkyl optionally substituted with one or more substituents independently selected from: halogen, —OR^{18A}, —N(R^{18A})₂, —C(O)R^{18A}, —C(O)N(R^{18A})₂, —NO₂, —CN, C₁₋₆ alkyl, and C₁₋₆ haloalkyl.

13. The compound or salt of claim 11, wherein two R¹ substituents on adjacent carbon atoms may come together to form a C₃₋₆ cycloalkyl optionally substituted with one or more substituents independently selected from: halogen, —OR^{18A}, —N(R^{18A})₂, —C(O)R^{18A}, —C(O)N(R^{18A})₂, —NO₂, —CN, C₁₋₆ alkyl, and C₁₋₆ haloalkyl.

14. The compound or salt of claim 11, wherein two R¹ substituents on different carbon atoms may come together to form a C₃₋₆ cycloalkyl optionally substituted with one or more substituents independently selected from: halogen, —OR^{18A}, —N(R^{18A})₂, —C(O)R^{18A}, —C(O)N(R^{18A})₂, —NO₂, —CN, C₁₋₆ alkyl, and C₁₋₆ haloalkyl.

15. The compound or salt of claim 1, wherein Formula (I) is represented by the structure of Formula (II): 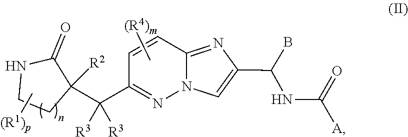
or a pharmaceutically acceptable salt thereof wherein:
A is selected from 5- to 6-membered heteroaryl and C₃₋₆ carbocycle, any of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹, —OC(O)R¹¹, —NO₂, ═O, ═N(R¹¹), and —CN;
B is selected from —C(H)(R⁵)₂ and C₃₋₁₀ carbocycle, wherein each C₃₋₁₀ carbocycle is optionally substituted with one or more substituents independently selected from:
halogen, —OR¹², —SR¹², —N(R¹²)₂, —C(O)R¹², —C(O)N(R¹²)₂, —N(R¹²)C(O)R¹²,
—N(R¹²)S(O)₂R¹², —C(O)OR¹², —OC(O)R¹², —S(O)R¹², —S(O)₂R¹², —NO₂, ═O, ═S, ═N(R¹²), —CN;
C₁₋₁₀ alkyl and C₃₋₁₀ carbocycle, any of which is optionally substituted with one or more substituents independently selected from halogen, —OR¹², —SR¹², —N(R¹²)₂,
—C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹²,
—N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁵ is independently selected at each occurrence from (i), (ii), and (iii):
(i) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
(ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³,
—N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
(iii) C₃₋₁₀ carbocycle optionally substituted with one or more substituents independently selected from:
halogen, —OR¹³, —SR¹³, —N(R¹³)₂, —C(O)R¹³, —C(O)N(R¹³)₂, —N(R¹³)C(O)R¹³,
—N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
C₁₋₁₀ alkyl optionally substituted with one or more substituents independently selected from halogen, —OR¹³, —SR¹³, —N(R¹³)₂, —C(O)R¹³, —C(O)N(R¹³)₂,
—N(R¹³)C(O)R¹³, —C(O)OR¹³, —OC(O)R¹³, —S(O)R¹³, —S(O)₂R¹³, —NO₂, ═O, ═S, ═N(R¹³), and —CN;
each R¹ is independently selected at each occurrence from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴,
—S(O)₂R¹⁴, —NO₂, ═O, ═S, ═N(R¹⁴), —CN; and C₁₋₁₀ alkyl optionally substituted with one or more substituents independently selected from halogen, —OR¹⁴, —SR¹⁴, —N(R¹⁴)₂, —C(O)R¹⁴,
—C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN;
R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵,
—OC(O)R¹⁵, —S(O)R¹⁵, —S(O)₂R¹⁵, —NO₂, ═O, ═S, ═N(R¹⁵), and —CN;
each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂,
—N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN; or
each R⁴ is independently selected at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷,
—S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂,
—N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN;
R¹¹, R¹², R¹³, R¹⁴, R¹⁵, R¹⁶, and R¹⁷ are each independently selected at each occurrence from:
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2;
m is selected from 0, 1 and 2; and
p is selected from 0, 1, 2, 3, 4, 5, and 6.

16. The compound or salt of claim 15, wherein n is 1.

17. The compound or salt of claim 15, wherein Formula (II) is represented by the structure of Formula (II-a):

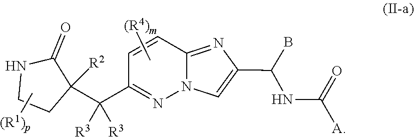

18. The compound or salt of claim 15, wherein n is 2.

19. The compound or salt of claim 15, wherein Formula (II) is represented by the structure of Formula (II-b):

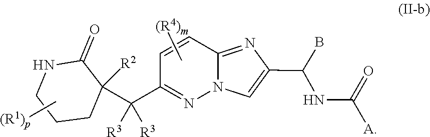

20. The compound or salt of claim 15, wherein A is 5-membered heteroaryl optionally substituted with one or more substituents independently selected from:
halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN; and
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

21. The compound or salt of claim 20, wherein A is selected from pyrazolyl, imidazolyl, oxazolyl, isoxazolyl, oxadiazolyl, triazolyl, and tetrazolyl, any one of which is optionally substituted with one or more C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

22. The compound or salt of claim 21, wherein A is selected from pyrazolyl and oxadiazolyl, each of which is optionally substituted with C₁₋₆ alkyl optionally substituted with one or more substituents independently selected from halogen, —OR¹¹, —N(R¹¹)₂, —C(O)R¹¹, —NO₂, and —CN.

23. The compound or salt of claim 22, wherein A is selected from pyrazolyl and oxadiazolyl, each of which is optionally substituted with methyl, ethyl, isopropyl, or propyl.

24. The compound or salt of claim 23, wherein A is selected from:

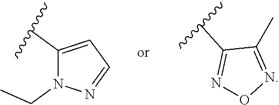

25. The compound or salt of claim 20, wherein A is selected from thiophenyl, thiadiazolyl, pyrrolyl, pyrazolyl, imidazolyl, oxazolyl, isoxazolyl, oxadiazolyl, triazolyl, and tetrazolyl, any one of which is optionally substituted with one or more substituents independently selected from:
halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN; and
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN, C₃₋₁₀ carbocycle, and 3- to 10-membered heterocycle; wherein the C₃₋₁₀ carbocycle and 3- to 10-membered heterocycle are each optionally substituted with one or more substituents selected from: —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

26. The compound or salt of claim 25, wherein A is selected from thiophenyl, thiadiazolyl, pyrrolyl, pyrazolyl, imidazolyl, oxazolyl, isoxazolyl, oxadiazolyl, and triazolyl, any one of which is optionally substituted with one or more substituents independently selected from halogen, —OR¹¹, —N(R¹¹)₂, —NO₂, —CN; and C₁₋₆ alkyl optionally substituted with one or more substituents independently selected from halogen, —OR¹¹, —N(R¹¹)₂, —NO₂, —CN, C₃₋₆ carbocycle and 3- to 6-membered heterocycle.

27. The compound or salt of claim 26, wherein A is selected from thiophenyl, thiadiazolyl, pyrrolyl, pyrazolyl, imidazolyl, oxazolyl, isoxazolyl, oxadiazolyl, and triazolyl, any one of which is optionally substituted with one or more substituents independently selected from —OR¹¹, —N(R¹¹)₂, —CN; and C₁₋₃ alkyl optionally substituted with one or more substituents independently selected from halogen, —OR¹¹, —N(R¹¹)₂, and C₃₋₆ cycloalkyl.

28. The compound or salt of claim 27, wherein A is selected from:

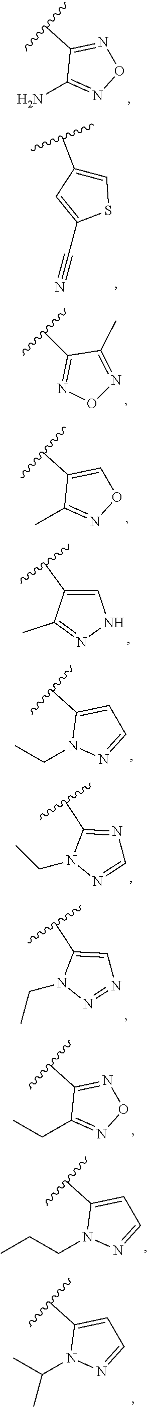

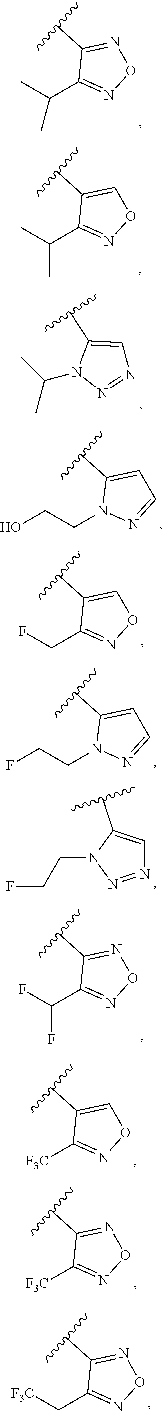

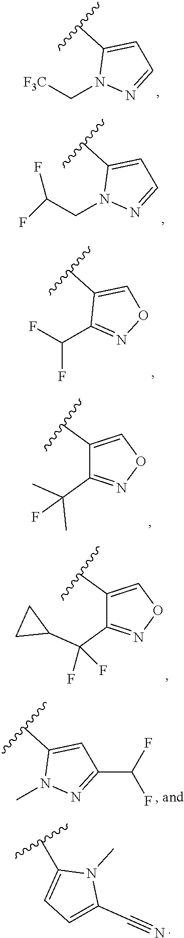

29. The compound or salt of claim 15, wherein A is C₃₋₆ carbocycle optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN; and C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

30. The compound or salt of claim 29, wherein the optionally substituted C₃₋₆ carbocycle of A is selected from spirocyclic C₄₋₆ carbocycle and bridged C₄₋₆ carbocycle, each of which is optionally substituted with one or more substituents independently selected from halogen, —OR¹¹, —N(R¹¹)₂—NO₂, —CN, C₁₋₆ alkyl, and C₁₋₆ haloalkyl.

31. The compound or salt of claim 30, wherein Ring A is

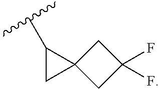 [SMILES: *C1CC12CC(F)(F)C2]

32. The compound or salt of claim 15, wherein B is —C(H)(R⁵)₂.

33. The compound or salt of claim 26, wherein each R⁵ of —C(H)(R⁵)₂ is independently selected from:
halogen, —OR¹³, —N(R¹³)₂, —C(O)R¹³, —C(O)OR¹³, —NO₂, —CN;
C₁₋₆ alkyl optionally substituted with one or more substituents independently selected from halogen, —OR¹³, —N(R¹³)₂, —C(O)R¹³, —C(O)OR¹³, —NO₂, ═O, ═S, ═N(R¹³), and —CN; and
C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl.

34. The compound or salt of claim 33, wherein each R⁵ of —C(H)(R⁵)₂ is independently selected from C₃₋₆ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl.

35. The compound or salt of claim 34, wherein —CH(R⁵)₂ is

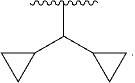 [SMILES: *C(C1CC1)C1CC1]

36. The compound or salt of claim 33, wherein each R⁵ of —C(H)(R⁵)₂ is independently selected from:
C₁₋₆ alkyl optionally substituted with one or more substituents independently selected from halogen, —OR¹³, —N(R¹³)₂, —C(O)R¹³, —C(O)OR¹³, —NO₂, ═O, ═S, ═N(R¹³), and —CN; and
C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl.

37. The compound or salt of claim 36, wherein each R⁵ of —C(H)(R⁵)₂ is independently selected from C₁₋₆ alkyl and C₃₋₆ carbocycle, each of which is optionally substituted with one or more substituents independently selected from halogen, —OR¹³, —N(R¹³)₂, —C(O)R¹³, —C(O)OR¹³, —NO₂, ═O, ═S, ═N(R¹³), and —CN.

38. The compound or salt of claim 37, wherein —CH(R⁵)₂ is selected from

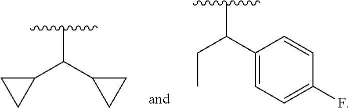 [SMILES: *C(C1CC1)C1CC1 ; *C(CC)c1ccc(F)cc1]

39. The compound or salt of claim 15, wherein B is C₆₋₁₀ carbocycle optionally substituted with one or more substituents independently selected from: halogen, —OR¹², N(R¹²)₂, —C(O)R¹², —NO₂, ═O, ═S, ═N(R¹²), —CN; and C₁₋₆ alkyl optionally substituted with one or more substituents independently selected from halogen, —OR¹², N(R¹²)₂, —C(O)R¹², —NO₂, ═O, ═S, ═N(R¹²), and —CN.

40. The compound or salt of claim 39, wherein B is selected from cyclohexyl and cycloheptyl, either of which is optionally substituted with one or more substituents independently selected from: halogen, —OR¹², N(R¹²)₂, —NO₂, —CN; and C₁₋₆ alkyl optionally substituted with one or more substituents independently selected from halogen, —OR¹², N(R¹²)₂, —C(O)R¹², —NO₂, ═O, ═S, ═N(R¹²), and —CN.

41. The compound or salt of claim 40, wherein B is selected from:

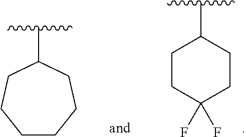 [SMILES: *C1CCCCCC1 ; *C1CCC(F)(F)CC1]

42. The compound or salt of claim 15, wherein B is selected from monocyclic C₃₋₇ carbocycle and bicyclic C₅₋₁₀ carbocycle, each of which is optionally substituted with one or more substituents independently selected from:
halogen, —OR¹², N(R¹²)₂, —C(O)R¹², —NO₂, ═O, ═S, ═N(R¹²), —CN; and
C₁₋₆ alkyl and C₃₋₁₀ carbocycle, each of which is optionally substituted with one or more substituents independently selected from halogen, —OR¹², N(R¹²)₂, —C(O)R¹², —NO₂, ═O, ═S, ═N(R¹²), and —CN.

43. The compound or salt of claim 42, wherein B is selected from monocyclic C₃₋₇ carbocycle optionally substituted with one or more substituents independently selected from halogen, —OR¹², N(R¹²)₂, —C(O)R¹², —NO₂, ═O, ═S, ═N(R¹²), —CN, C₁₋₆ alkyl, C₁₋₆ haloalkyl and C₃₋₆ cycloalkyl.

44. The compound or salt of claim 43, wherein B is selected from cyclopropyl, cyclobutyl, cyclopentyl, cyclohexyl, and cycloheptyl, any of which is optionally substituted with one or more substituents independently selected from halogen, C₁₋₆ alkyl, C₁₋₆ haloalkyl and C₃₋₆ cycloalkyl.

45. The compound or salt of claim 44, wherein B is selected from

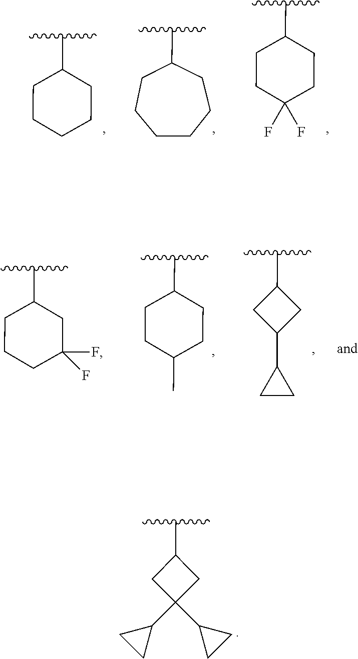

46. The compound or salt of claim 42, wherein B is selected from bicyclic C₅₋₁₀ carbocycle optionally substituted with one or more substituents independently selected from halogen, —OR¹², N(R¹²)₂, —C(O)R¹², —NO₂, ═O, ═S, ═N(R¹²), —CN, C₁₋₆ alkyl, and C₁₋₆ haloalkyl.

47. The compound or salt of claim 46, wherein the bicyclic C₅₋₁₀ carbocycle of B is selected from fused C₅₋₁₀ carbocycle, bridged C₅₋₁₀ carbocycle, and spirocyclic C₅₋₁₀ carbocycle, any of which is optionally substituted with one or more substituents independently selected from halogen, C₁₋₆ alkyl, and C₁₋₆ haloalkyl.

48. The compound or salt of claim 47, wherein the bicyclic C₅₋₁₀ carbocycle of B is selected from fused C₅₋₁₀ carbocycle optionally substituted with one or more substituents independently selected from halogen, C₁₋₆ alkyl, and C₁₋₆ haloalkyl.

49. The compound or salt of claim 48, wherein B is selected from

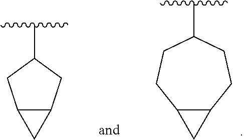 [SMILES: *C1CC2CC2C1 ; *C1CCC2CC2CC1]

50. The compound or salt of claim 47, wherein the bicyclic C₅₋₁₀ carbocycle of B is selected from bridged C₅₋₁₀ carbocycle optionally substituted with one or more substituents independently selected from halogen, C₁₋₆ alkyl, and C₁₋₆ haloalkyl.

51. The compound or salt of claim 50, wherein B is

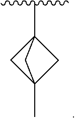 [SMILES: *C12CC(C)(C1)C2]

52. The compound or salt of claim 47, wherein the bicyclic C₅₋₁₀ carbocycle of B is selected from spirocyclic C₅₋₁₀ carbocycle optionally substituted with one or more substituents independently selected from halogen, C₁₋₆ alkyl, C₁₋₆ haloalkyl.

53. The compound or salt of claim 52, wherein B is selected from

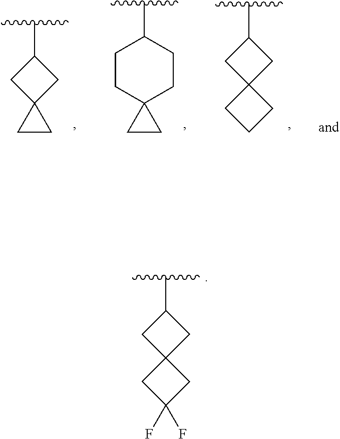 [SMILES: *C1CC2(CC2)C1 ; *C1CCC2(CC1)CC2 ; *C1CC2(CCC2)C1 ; *C1CC2(C1)CC(F)(F)C2]

54. The compound or salt of claim 15, wherein m is 0.

55. The compound or salt of claim 15, wherein m is 1; and R⁴ is selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, and —CN.

56. The compound or salt of claim 55, wherein R⁴ is selected from halogen, —OR¹⁷, —N(R¹⁷)₂, —C(O)R¹⁷, —S(O)₂R¹⁷, and —CN; and R¹⁷ is selected at each occurrence from:
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₆ carbocycle and 3- to 6-membered heterocycle, wherein each C₃₋₆ carbocycle and 3- to 6-membered heterocycle; and
C₃₋₆ carbocycle and 3- to 6-membered heterocycle optionally substituted with one or more substituents independently selected from halogen, —OH, —O—C₁₋₆ alkyl, —O—C₁₋₆ haloalkyl, —NH₂, —NO₂, ═O, and —CN.

57. The compound or salt of claim 56, wherein R⁴ is selected from —Cl, —CN,

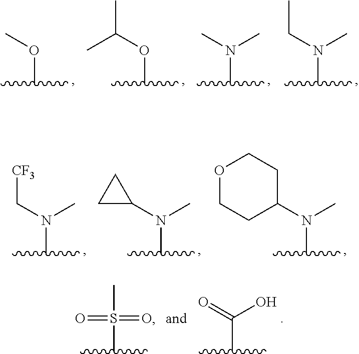

58. The compound or salt of claim 55, wherein R⁴ is C₁₋₆ alkyl optionally substituted with one or more substituents independently selected from halogen, —OR¹⁷, and —CN; and R¹⁷ is selected at each occurrence from halogen, C₁₋₆ alkyl, and C₁₋₆ haloalkyl.

59. The compound or salt of claim 58, wherein R⁴ is selected from methyl,

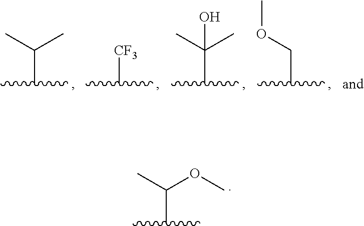

60. The compound or salt of claim 15, wherein each R³ is independently selected at each occurrence from hydrogen, halogen, —OR¹⁶, —N(R¹⁶)₂, —NO₂, —CN, C₁₋₃ alkyl, and C₁₋₃ haloalkyl.

61. The compound or salt of claim 55, wherein each R³ is hydrogen.

62. The compound or salt of claim 60, wherein each R³ is independently selected at each occurrence from hydrogen, methyl, ethyl, and

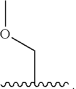 [SMILES: *COC ; "."]

63. The compound or salt of claim 15, wherein R² is hydrogen.

64. The compound or salt of claim 15, wherein R² is selected from hydrogen, C₁₋₃ alkyl, and C₁₋₃ haloalkyl.

65. The compound or salt of claim 15, wherein p is selected from 0, 1, 2, 3, and 4.

66. The compound or salt of claim 15, wherein each R¹ is independently selected at each occurrence from halogen, —OR¹⁴, —N(R¹⁴)₂, —NO₂, —CN, C₁₋₆ alkyl, and C₁₋₆ haloalkyl.

67. The compound or salt of claim 66, wherein each R¹ is independently selected at each occurrence from halogen and —CF₃.

68. The compound or salt of claim 66, wherein each R¹ is independently selected at each occurrence from halogen, C₁₋₆ alkyl, and C₁₋₆ haloalkyl.

69. The compound or salt of claim 68, wherein each R¹ is independently selected at each occurrence from methyl, ethyl,

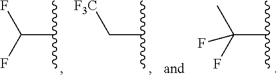

70. The compound or salt of claim 15, wherein Formula (I) is selected from: 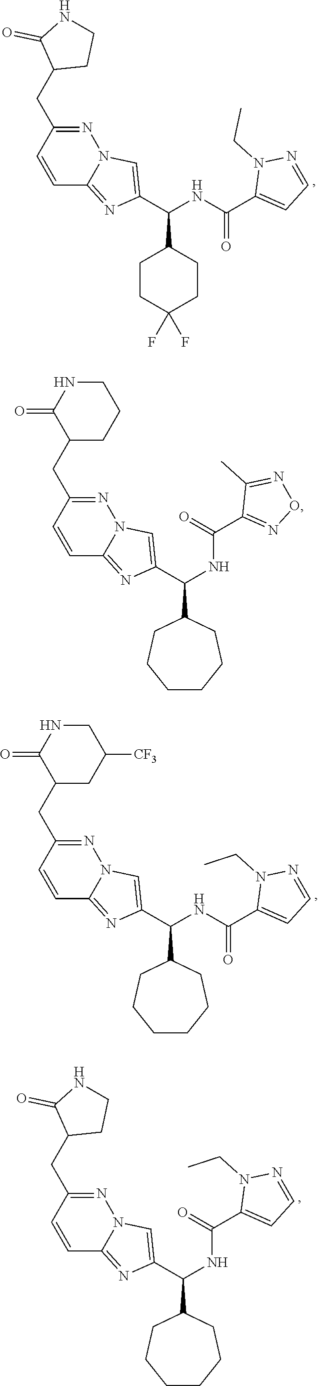 [SMILES: CCn1nccc1C(=O)N[C@H](c1cn2nc(CC3CCNC3=O)ccc2n1)C1CCC(F)(F)CC1 ; Cc1nonc1C(=O)N[C@H](c1cn2nc(CC3CCCNC3=O)ccc2n1)C1CCCCCC1 ; CCn1nccc1C(=O)N[C@H](c1cn2nc(CC3CC(C(F)(F)F)CNC3=O)ccc2n1)C1CCCCCC1 ; CCn1nccc1C(=O)N[C@H](c1cn2nc(CC3CCNC3=O)ccc2n1)C1CCCCCC1] 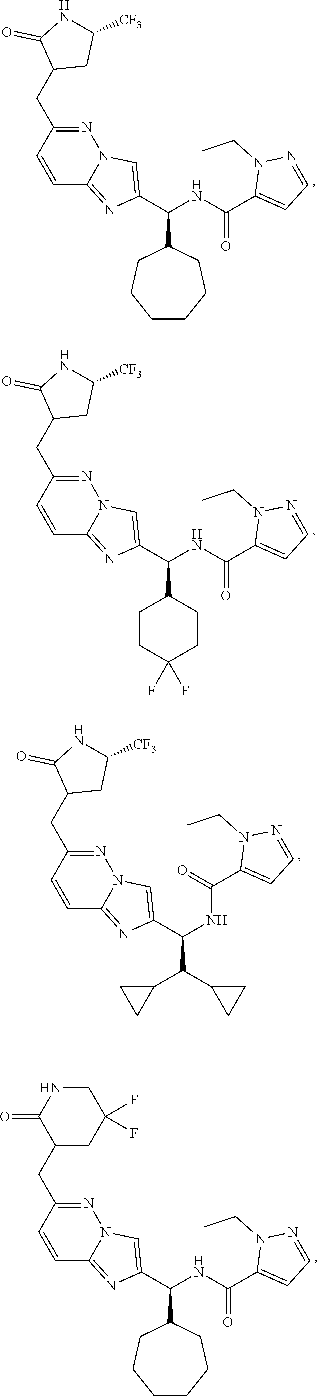 [SMILES: CCn1nccc1C(=O)N[C@H](c1cn2nc(CC3C[C@@H](C(F)(F)F)NC3=O)ccc2n1)C1CCCCCC1 ; CCn1nccc1C(=O)N[C@H](c1cn2nc(CC3C[C@@H](C(F)(F)F)NC3=O)ccc2n1)C1CCC(F)(F)CC1 ; CCn1nccc1C(=O)N[C@H](c1cn2nc(CC3C[C@@H](C(F)(F)F)NC3=O)ccc2n1)C(C1CC1)C1CC1 ; CCn1nccc1C(=O)N[C@H](c1cn2nc(CC3CC(F)(F)CNC3=O)ccc2n1)C1CCCCCC1] 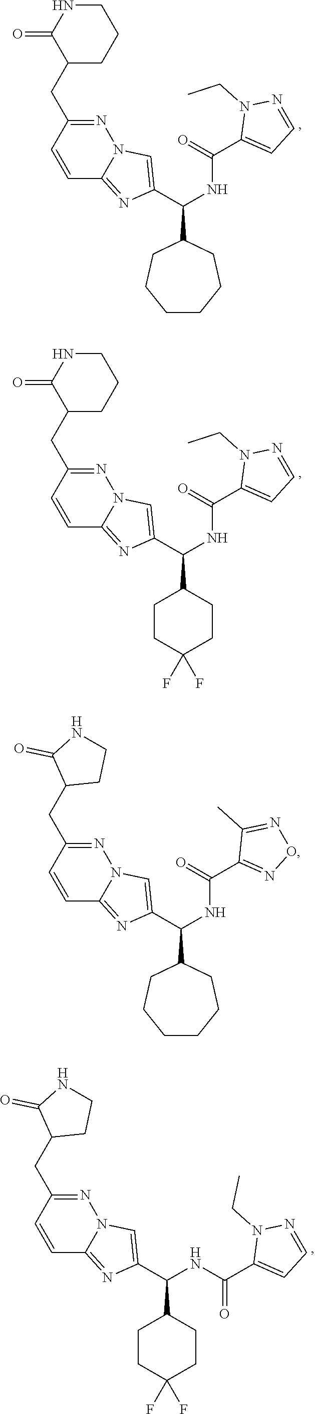 [SMILES: CCn1nccc1C(=O)N[C@H](c1cn2nc(CC3CCCNC3=O)ccc2n1)C1CCCCCC1 ; CCn1nccc1C(=O)N[C@H](c1cn2nc(CC3CCCNC3=O)ccc2n1)C1CCC(F)(F)CC1 ; Cc1nonc1C(=O)N[C@H](c1cn2nc(CC3CCNC3=O)ccc2n1)C1CCCCCC1 ; CCn1nccc1C(=O)N[C@H](c1cn2nc(CC3CCNC3=O)ccc2n1)C1CCC(F)(F)CC1] 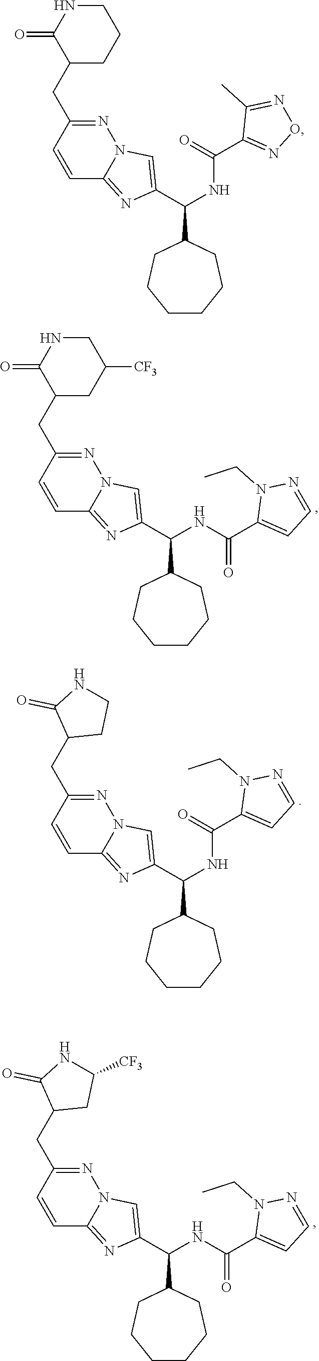 [SMILES: Cc1nonc1C(=O)N[C@H](c1cn2nc(CC3CCCNC3=O)ccc2n1)C1CCCCCC1 ; CCn1nccc1C(=O)N[C@H](c1cn2nc(CC3CC(C(F)(F)F)CNC3=O)ccc2n1)C1CCCCCC1 ; CCn1nccc1C(=O)N[C@H](c1cn2nc(CC3CCNC3=O)ccc2n1)C1CCCCCC1 ; CCn1nccc1C(=O)N[C@H](c1cn2nc(CC3C[C@@H](C(F)(F)F)NC3=O)ccc2n1)C1CCCCCC1] 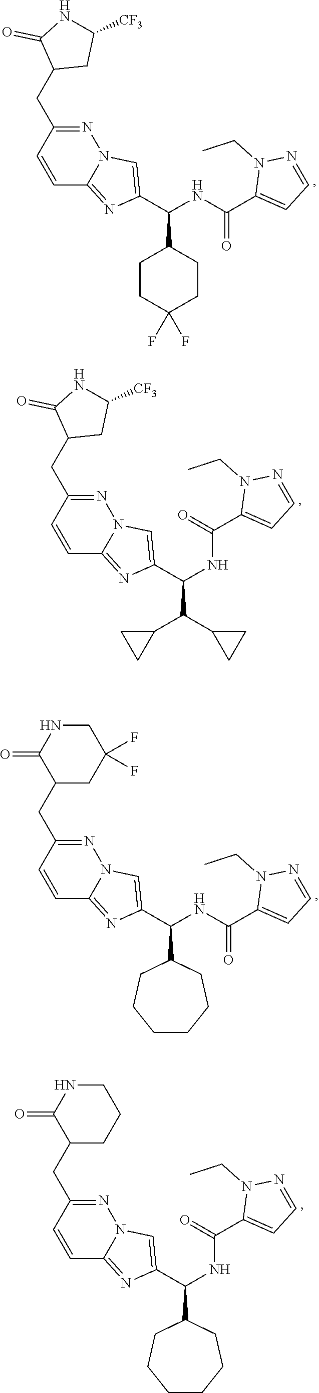 [SMILES: CCn1nccc1C(=O)N[C@H](c1cn2nc(CC3C[C@@H](C(F)(F)F)NC3=O)ccc2n1)C1CCC(F)(F)CC1 ; CCn1nccc1C(=O)N[C@H](c1cn2nc(CC3C[C@@H](C(F)(F)F)NC3=O)ccc2n1)C(C1CC1)C1CC1 ; CCn1nccc1C(=O)N[C@H](c1cn2nc(CC3CC(F)(F)CNC3=O)ccc2n1)C1CCCCCC1 ; CCn1nccc1C(=O)N[C@H](c1cn2nc(CC3CCCNC3=O)ccc2n1)C1CCCCCC1] 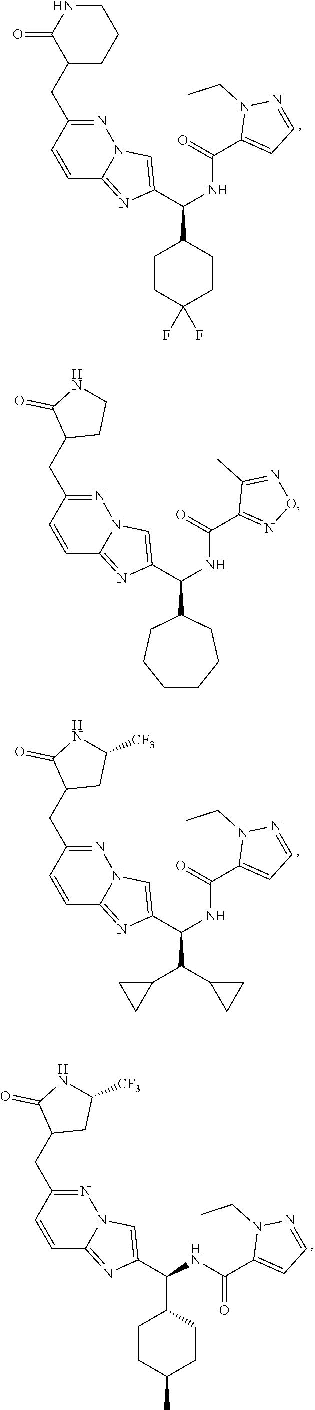 [SMILES: CCn1nccc1C(=O)N[C@H](c1cn2nc(CC3CCCNC3=O)ccc2n1)C1CCC(F)(F)CC1 ; Cc1nonc1C(=O)N[C@H](c1cn2nc(CC3CCNC3=O)ccc2n1)C1CCCCCC1 ; CCn1nccc1C(=O)N[C@H](c1cn2nc(CC3C[C@@H](C(F)(F)F)NC3=O)ccc2n1)C(C1CC1)C1CC1 ; CCn1nccc1C(=O)N[C@H](c1cn2nc(CC3C[C@@H](C(F)(F)F)NC3=O)ccc2n1)[C@H]1CC[C@H](C)CC1] 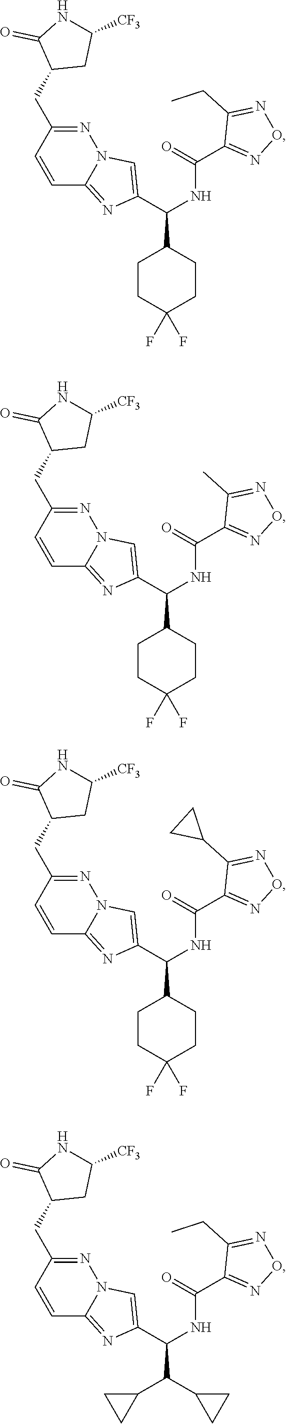 [SMILES: CCc1nonc1C(=O)N[C@H](c1cn2nc(C[C@H]3C[C@@H](C(F)(F)F)NC3=O)ccc2n1)C1CCC(F)(F)CC1 ; Cc1nonc1C(=O)N[C@H](c1cn2nc(C[C@H]3C[C@@H](C(F)(F)F)NC3=O)ccc2n1)C1CCC(F)(F)CC1 ; O=C(N[C@H](c1cn2nc(C[C@H]3C[C@@H](C(F)(F)F)NC3=O)ccc2n1)C1CCC(F)(F)CC1)c1nonc1C1CC1 ; CCc1nonc1C(=O)N[C@H](c1cn2nc(C[C@H]3C[C@@H](C(F)(F)F)NC3=O)ccc2n1)C(C1CC1)C1CC1] 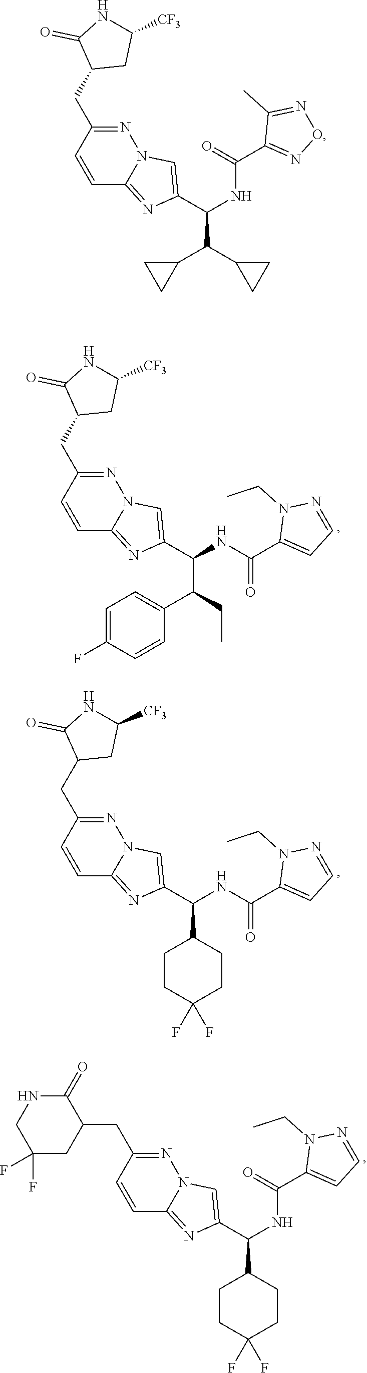 [SMILES: Cc1nonc1C(=O)N[C@H](c1cn2nc(C[C@H]3C[C@@H](C(F)(F)F)NC3=O)ccc2n1)C(C1CC1)C1CC1 ; CC[C@@H](c1ccc(F)cc1)[C@H](NC(=O)c1ccnn1CC)c1cn2nc(C[C@H]3C[C@@H](C(F)(F)F)NC3=O)ccc2n1 ; CCn1nccc1C(=O)N[C@H](c1cn2nc(CC3C[C@H](C(F)(F)F)NC3=O)ccc2n1)C1CCC(F)(F)CC1 ; CCn1nccc1C(=O)N[C@H](c1cn2nc(CC3CC(F)(F)CNC3=O)ccc2n1)C1CCC(F)(F)CC1] 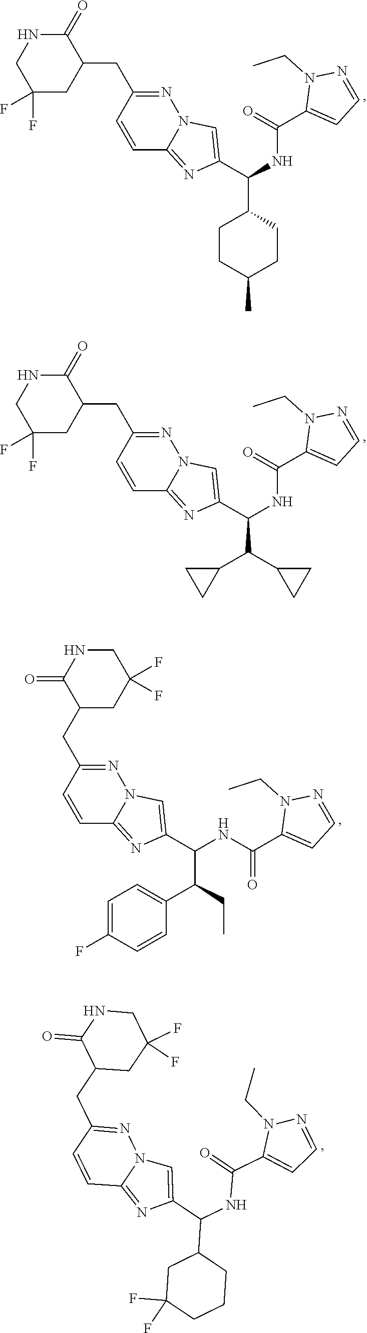 [SMILES: CCn1nccc1C(=O)N[C@H](c1cn2nc(CC3CC(F)(F)CNC3=O)ccc2n1)[C@H]1CC[C@H](C)CC1 ; CCn1nccc1C(=O)N[C@H](c1cn2nc(CC3CC(F)(F)CNC3=O)ccc2n1)C(C1CC1)C1CC1 ; CC[C@@H](c1ccc(F)cc1)C(NC(=O)c1ccnn1CC)c1cn2nc(CC3CC(F)(F)CNC3=O)ccc2n1 ; CCn1nccc1C(=O)NC(c1cn2nc(CC3CC(F)(F)CNC3=O)ccc2n1)C1CCCC(F)(F)C1] 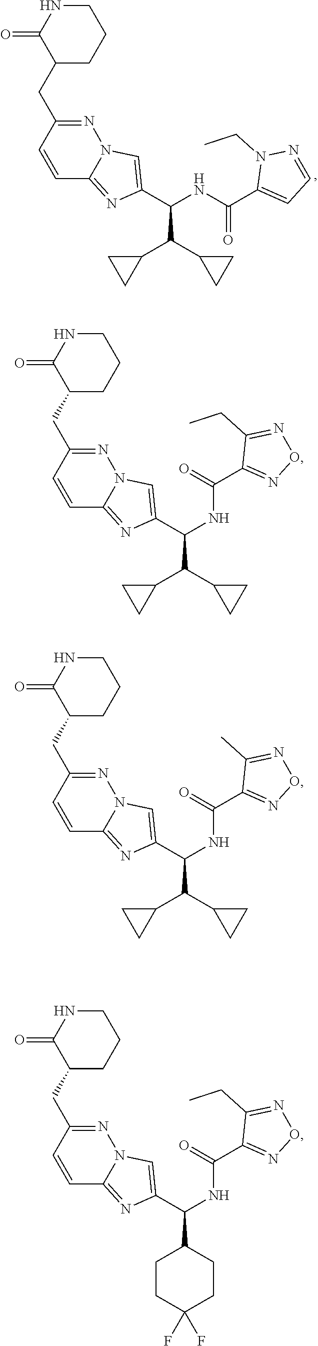 [SMILES: CCn1nccc1C(=O)N[C@H](c1cn2nc(CC3CCCNC3=O)ccc2n1)C(C1CC1)C1CC1 ; CCc1nonc1C(=O)N[C@H](c1cn2nc(C[C@H]3CCCNC3=O)ccc2n1)C(C1CC1)C1CC1 ; Cc1nonc1C(=O)N[C@H](c1cn2nc(C[C@H]3CCCNC3=O)ccc2n1)C(C1CC1)C1CC1 ; CCc1nonc1C(=O)N[C@H](c1cn2nc(C[C@H]3CCCNC3=O)ccc2n1)C1CCC(F)(F)CC1] 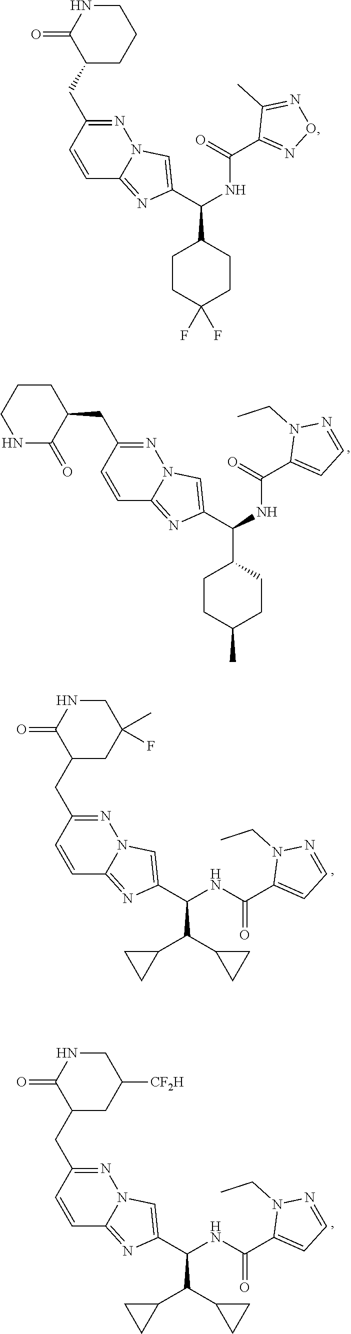 [SMILES: Cc1nonc1C(=O)N[C@H](c1cn2nc(C[C@H]3CCCNC3=O)ccc2n1)C1CCC(F)(F)CC1 ; CCn1nccc1C(=O)N[C@H](c1cn2nc(C[C@@H]3CCCNC3=O)ccc2n1)[C@H]1CC[C@H](C)CC1 ; CCn1nccc1C(=O)N[C@H](c1cn2nc(CC3CC(C)(F)CNC3=O)ccc2n1)C(C1CC1)C1CC1 ; CCn1nccc1C(=O)N[C@H](c1cn2nc(CC3CC(C(F)F)CNC3=O)ccc2n1)C(C1CC1)C1CC1] 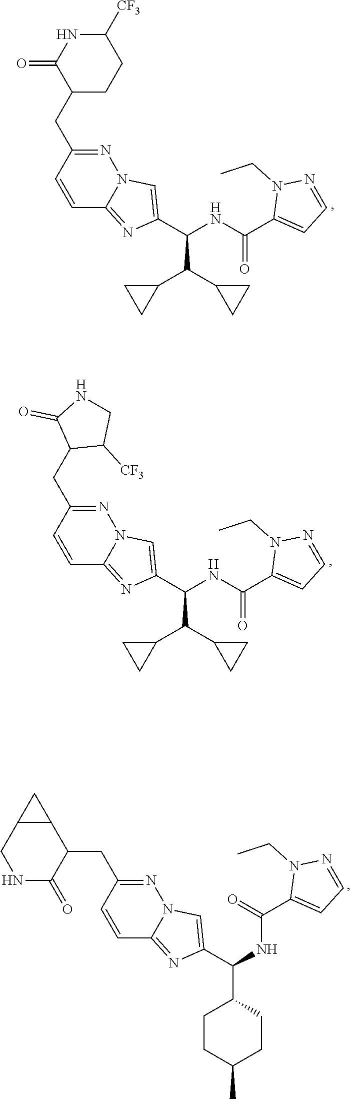 [SMILES: CCn1nccc1C(=O)N[C@H](c1cn2nc(CC3CCC(C(F)(F)F)NC3=O)ccc2n1)C(C1CC1)C1CC1 ; CCn1nccc1C(=O)N[C@H](c1cn2nc(CC3C(=O)NCC3C(F)(F)F)ccc2n1)C(C1CC1)C1CC1 ; CCn1nccc1C(=O)N[C@H](c1cn2nc(CC3C(=O)NCC4CC43)ccc2n1)[C@H]1CC[C@H](C)CC1] 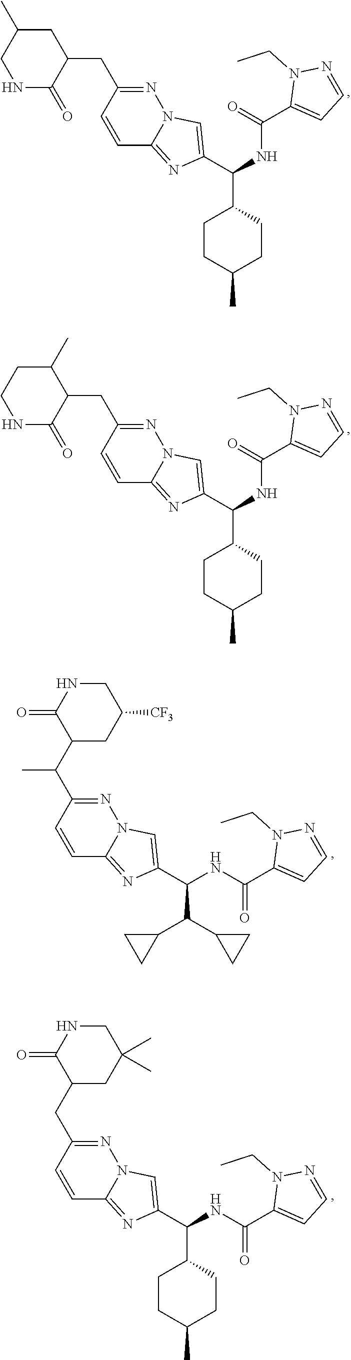 [SMILES: CCn1nccc1C(=O)N[C@H](c1cn2nc(CC3CC(C)CNC3=O)ccc2n1)[C@H]1CC[C@H](C)CC1 ; CCn1nccc1C(=O)N[C@H](c1cn2nc(CC3C(=O)NCCC3C)ccc2n1)[C@H]1CC[C@H](C)CC1 ; CCn1nccc1C(=O)N[C@H](c1cn2nc(C(C)C3C[C@@H](C(F)(F)F)CNC3=O)ccc2n1)C(C1CC1)C1CC1 ; CCn1nccc1C(=O)N[C@H](c1cn2nc(CC3CC(C)(C)CNC3=O)ccc2n1)[C@H]1CC[C@H](C)CC1] 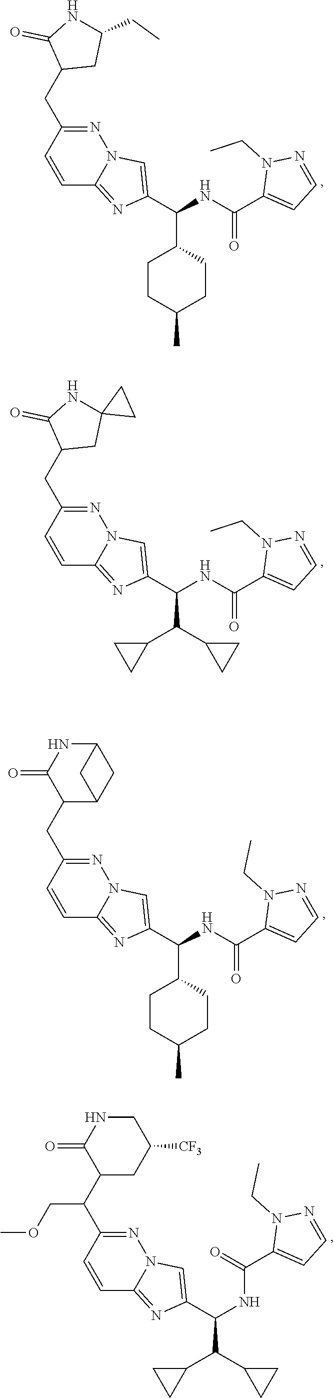 [SMILES: CC[C@@H]1CC(Cc2ccc3nc([C@@H](NC(=O)c4ccnn4CC)[C@H]4CC[C@H](C)CC4)cn3n2)C(=O)N1 ; CCn1nccc1C(=O)N[C@H](c1cn2nc(CC3CC4(CC4)NC3=O)ccc2n1)C(C1CC1)C1CC1 ; CCn1nccc1C(=O)N[C@H](c1cn2nc(CC3C(=O)NC4CC3C4)ccc2n1)[C@H]1CC[C@H](C)CC1 ; CCn1nccc1C(=O)N[C@H](c1cn2nc(C(COC)C3C[C@@H](C(F)(F)F)CNC3=O)ccc2n1)C(C1CC1)C1CC1] 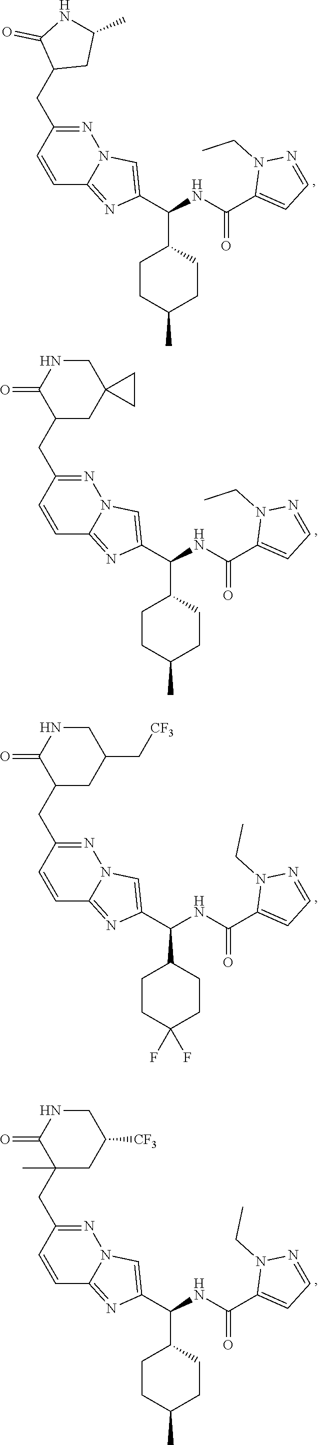 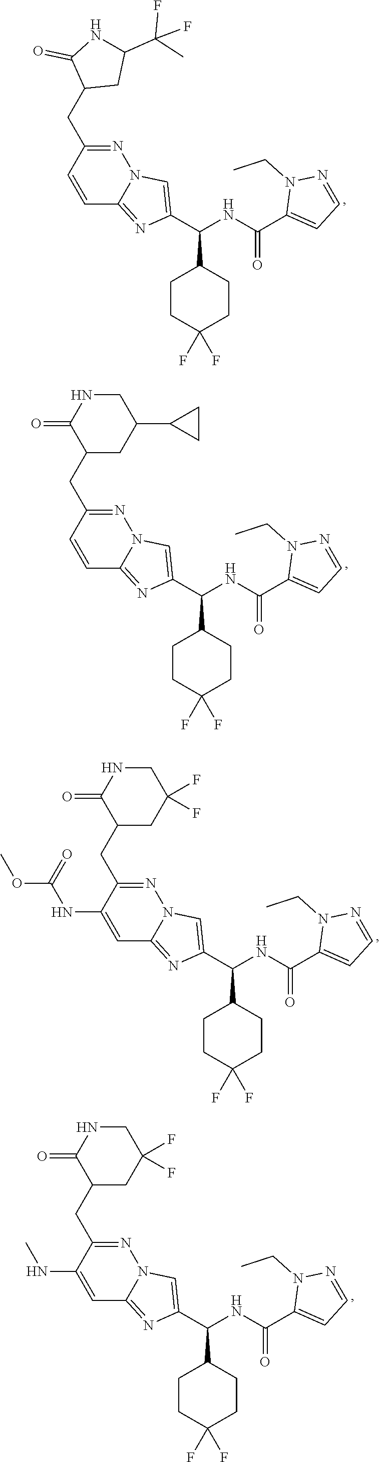 [SMILES: CCn1nccc1C(=O)N[C@H](c1cn2nc(CC3CC(C(C)(F)F)NC3=O)ccc2n1)C1CCC(F)(F)CC1 ; CCn1nccc1C(=O)N[C@H](c1cn2nc(CC3CC(C4CC4)CNC3=O)ccc2n1)C1CCC(F)(F)CC1 ; CCn1nccc1C(=O)N[C@H](c1cn2nc(CC3CC(F)(F)CNC3=O)c(NC(=O)OC)cc2n1)C1CCC(F)(F)CC1 ; CCn1nccc1C(=O)N[C@H](c1cn2nc(CC3CC(F)(F)CNC3=O)c(NC)cc2n1)C1CCC(F)(F)CC1] 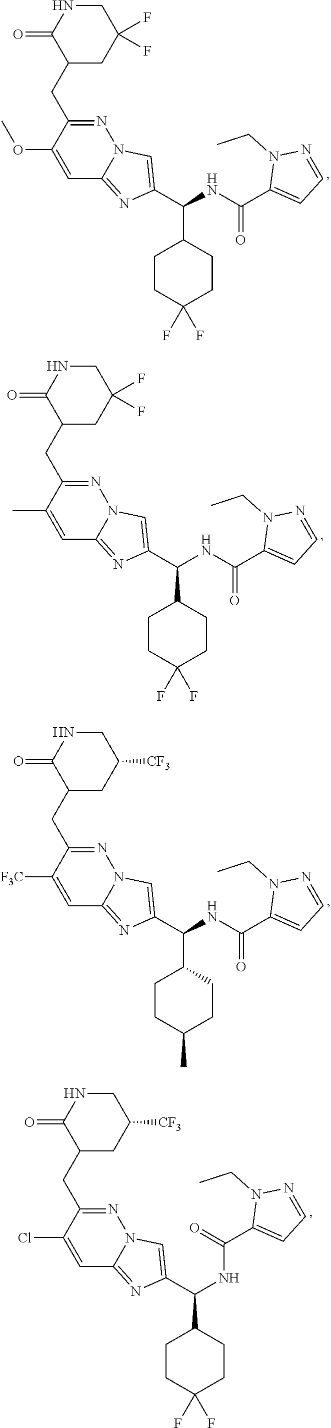 [SMILES: CCn1nccc1C(=O)N[C@H](c1cn2nc(CC3CC(F)(F)CNC3=O)c(OC)cc2n1)C1CCC(F)(F)CC1 ; CCn1nccc1C(=O)N[C@H](c1cn2nc(CC3CC(F)(F)CNC3=O)c(C)cc2n1)C1CCC(F)(F)CC1 ; CCn1nccc1C(=O)N[C@H](c1cn2nc(CC3C[C@@H](C(F)(F)F)CNC3=O)c(C(F)(F)F)cc2n1)[C@H]1CC[C@H](C)CC1 ; CCn1nccc1C(=O)N[C@H](c1cn2nc(CC3C[C@@H](C(F)(F)F)CNC3=O)c(Cl)cc2n1)C1CCC(F)(F)CC1] 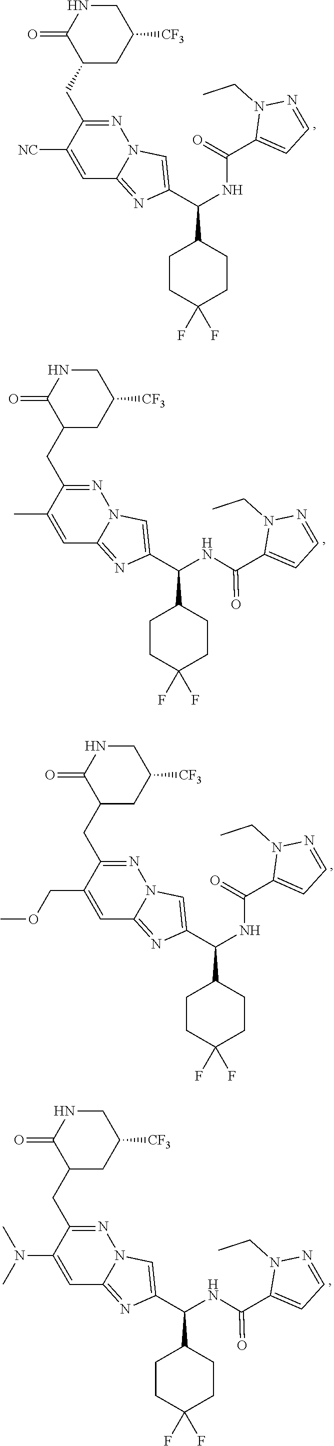 [SMILES: CCn1nccc1C(=O)N[C@H](c1cn2nc(C[C@H]3C[C@@H](C(F)(F)F)CNC3=O)c(C#N)cc2n1)C1CCC(F)(F)CC1 ; CCn1nccc1C(=O)N[C@H](c1cn2nc(CC3C[C@@H](C(F)(F)F)CNC3=O)c(C)cc2n1)C1CCC(F)(F)CC1 ; CCn1nccc1C(=O)N[C@H](c1cn2nc(CC3C[C@@H](C(F)(F)F)CNC3=O)c(COC)cc2n1)C1CCC(F)(F)CC1 ; CCn1nccc1C(=O)N[C@H](c1cn2nc(CC3C[C@@H](C(F)(F)F)CNC3=O)c(N(C)C)cc2n1)C1CCC(F)(F)CC1] 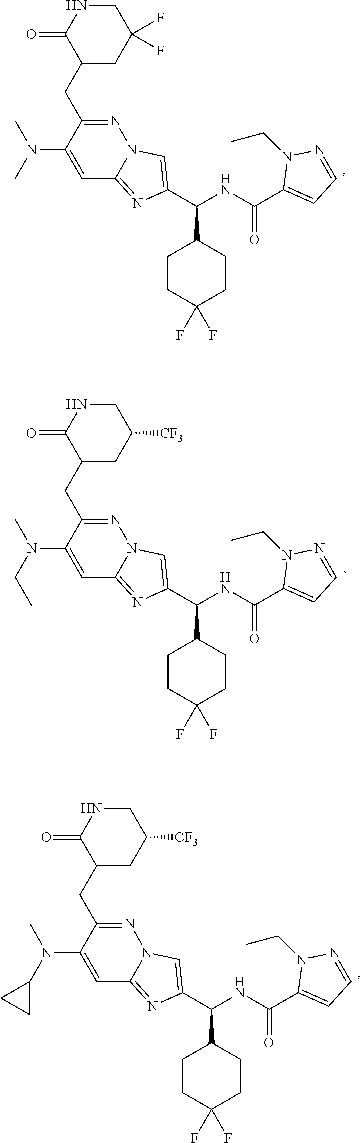 [SMILES: CCn1nccc1C(=O)N[C@H](c1cn2nc(CC3CC(F)(F)CNC3=O)c(N(C)C)cc2n1)C1CCC(F)(F)CC1 ; CCN(C)c1cc2nc([C@@H](NC(=O)c3ccnn3CC)C3CCC(F)(F)CC3)cn2nc1CC1C[C@@H](C(F)(F)F)CNC1=O ; CCn1nccc1C(=O)N[C@H](c1cn2nc(CC3C[C@@H](C(F)(F)F)CNC3=O)c(N(C)C3CC3)cc2n1)C1CCC(F)(F)CC1] 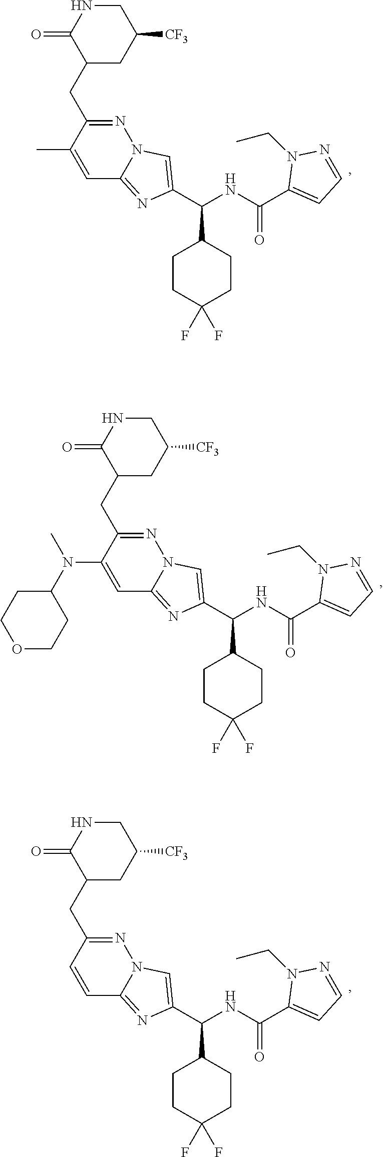 [SMILES: CCn1nccc1C(=O)N[C@H](c1cn2nc(CC3C[C@H](C(F)(F)F)CNC3=O)c(C)cc2n1)C1CCC(F)(F)CC1 ; CCn1nccc1C(=O)N[C@H](c1cn2nc(CC3C[C@@H](C(F)(F)F)CNC3=O)c(N(C)C3CCOCC3)cc2n1)C1CCC(F)(F)CC1 ; CCn1nccc1C(=O)N[C@H](c1cn2nc(CC3C[C@@H](C(F)(F)F)CNC3=O)ccc2n1)C1CCC(F)(F)CC1] 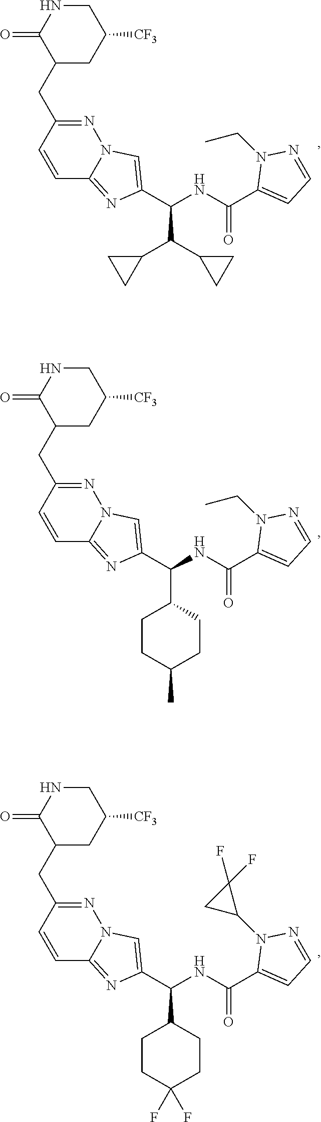 [SMILES: CCn1nccc1C(=O)N[C@H](c1cn2nc(CC3C[C@@H](C(F)(F)F)CNC3=O)ccc2n1)C(C1CC1)C1CC1 ; CCn1nccc1C(=O)N[C@H](c1cn2nc(CC3C[C@@H](C(F)(F)F)CNC3=O)ccc2n1)[C@H]1CC[C@H](C)CC1 ; O=C(N[C@H](c1cn2nc(CC3C[C@@H](C(F)(F)F)CNC3=O)ccc2n1)C1CCC(F)(F)CC1)c1ccnn1C1CC1(F)F] 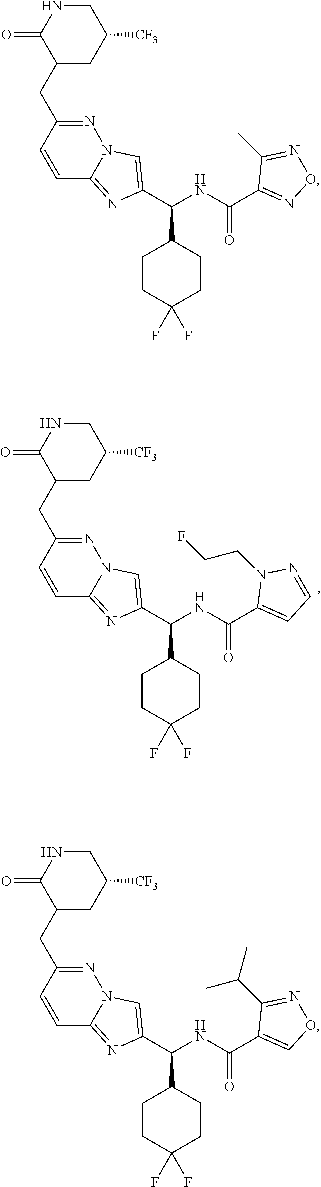 [SMILES: Cc1nonc1C(=O)N[C@H](c1cn2nc(CC3C[C@@H](C(F)(F)F)CNC3=O)ccc2n1)C1CCC(F)(F)CC1 ; O=C(N[C@H](c1cn2nc(CC3C[C@@H](C(F)(F)F)CNC3=O)ccc2n1)C1CCC(F)(F)CC1)c1ccnn1CCF ; CC(C)c1nocc1C(=O)N[C@H](c1cn2nc(CC3C[C@@H](C(F)(F)F)CNC3=O)ccc2n1)C1CCC(F)(F)CC1] 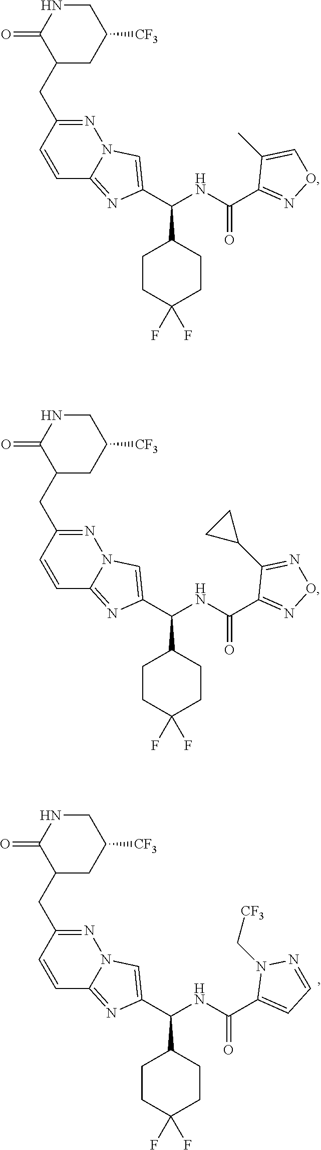 [SMILES: Cc1conc1C(=O)N[C@H](c1cn2nc(CC3C[C@@H](C(F)(F)F)CNC3=O)ccc2n1)C1CCC(F)(F)CC1 ; O=C(N[C@H](c1cn2nc(CC3C[C@@H](C(F)(F)F)CNC3=O)ccc2n1)C1CCC(F)(F)CC1)c1nonc1C1CC1 ; O=C(N[C@H](c1cn2nc(CC3C[C@@H](C(F)(F)F)CNC3=O)ccc2n1)C1CCC(F)(F)CC1)c1ccnn1CC(F)(F)F] 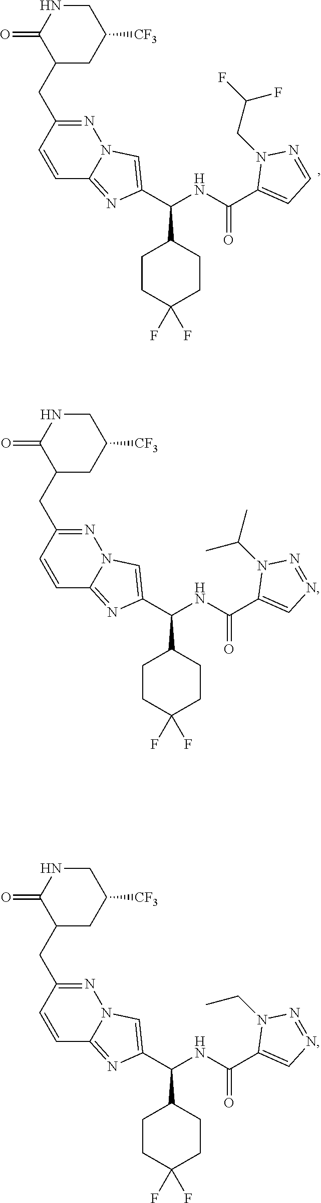 [SMILES: O=C(N[C@H](c1cn2nc(CC3C[C@@H](C(F)(F)F)CNC3=O)ccc2n1)C1CCC(F)(F)CC1)c1ccnn1CC(F)F ; CC(C)n1nncc1C(=O)N[C@H](c1cn2nc(CC3C[C@@H](C(F)(F)F)CNC3=O)ccc2n1)C1CCC(F)(F)CC1 ; CCn1nncc1C(=O)N[C@H](c1cn2nc(CC3C[C@@H](C(F)(F)F)CNC3=O)ccc2n1)C1CCC(F)(F)CC1] 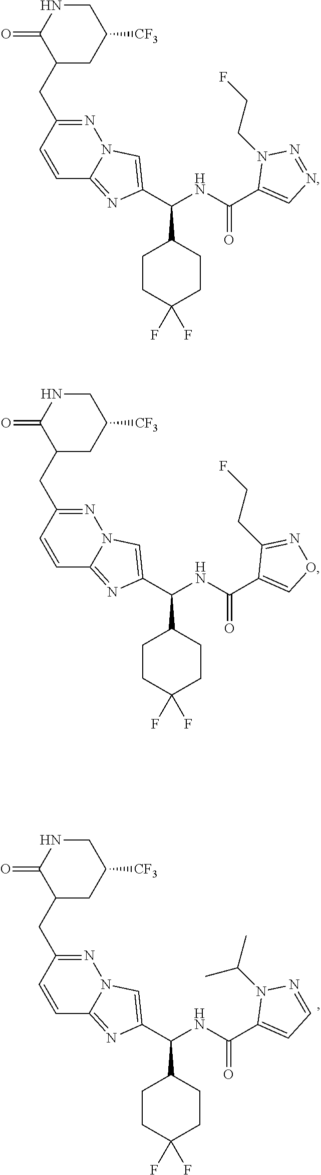 [SMILES: O=C(N[C@H](c1cn2nc(CC3C[C@@H](C(F)(F)F)CNC3=O)ccc2n1)C1CCC(F)(F)CC1)c1cnnn1CCF ; O=C(N[C@H](c1cn2nc(CC3C[C@@H](C(F)(F)F)CNC3=O)ccc2n1)C1CCC(F)(F)CC1)c1conc1CCF ; CC(C)n1nccc1C(=O)N[C@H](c1cn2nc(CC3C[C@@H](C(F)(F)F)CNC3=O)ccc2n1)C1CCC(F)(F)CC1] 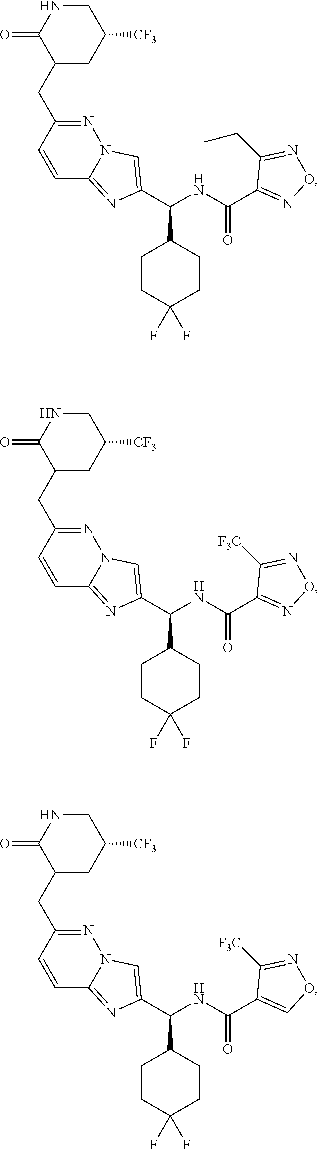 [SMILES: CCc1nonc1C(=O)N[C@H](c1cn2nc(CC3C[C@@H](C(F)(F)F)CNC3=O)ccc2n1)C1CCC(F)(F)CC1 ; O=C(N[C@H](c1cn2nc(CC3C[C@@H](C(F)(F)F)CNC3=O)ccc2n1)C1CCC(F)(F)CC1)c1nonc1C(F)(F)F ; O=C(N[C@H](c1cn2nc(CC3C[C@@H](C(F)(F)F)CNC3=O)ccc2n1)C1CCC(F)(F)CC1)c1conc1C(F)(F)F] 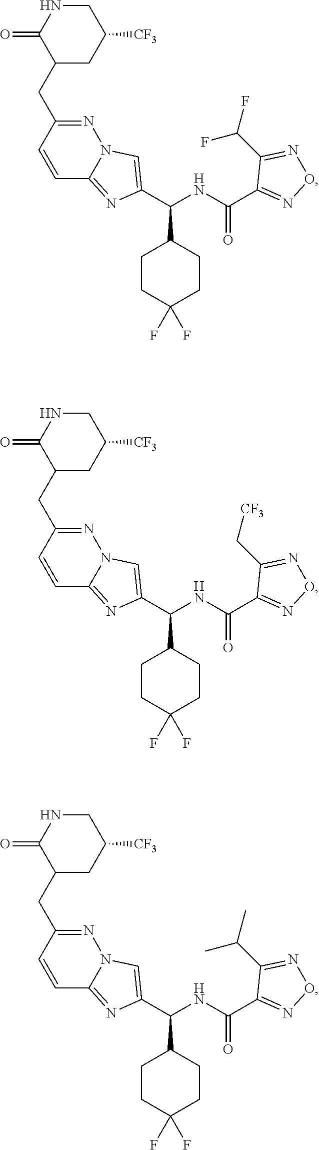 [SMILES: O=C(N[C@H](c1cn2nc(CC3C[C@@H](C(F)(F)F)CNC3=O)ccc2n1)C1CCC(F)(F)CC1)c1nonc1C(F)F ; O=C(N[C@H](c1cn2nc(CC3C[C@@H](C(F)(F)F)CNC3=O)ccc2n1)C1CCC(F)(F)CC1)c1nonc1CC(F)(F)F ; CC(C)c1nonc1C(=O)N[C@H](c1cn2nc(CC3C[C@@H](C(F)(F)F)CNC3=O)ccc2n1)C1CCC(F)(F)CC1] 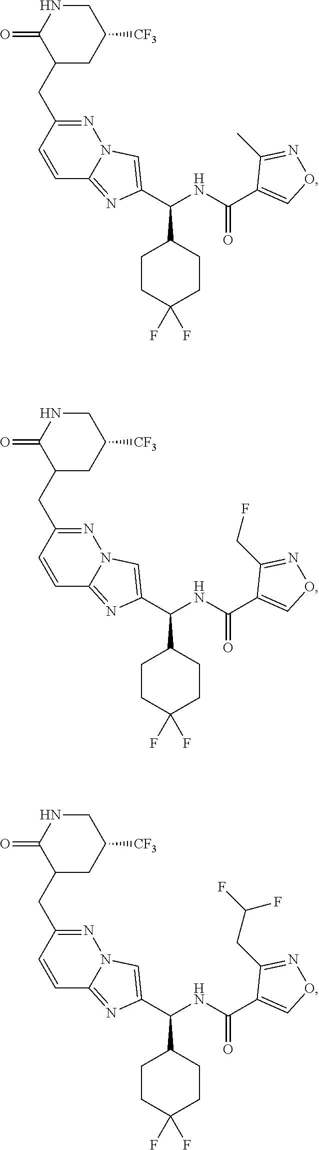 [SMILES: Cc1nocc1C(=O)N[C@H](c1cn2nc(CC3C[C@@H](C(F)(F)F)CNC3=O)ccc2n1)C1CCC(F)(F)CC1 ; O=C(N[C@H](c1cn2nc(CC3C[C@@H](C(F)(F)F)CNC3=O)ccc2n1)C1CCC(F)(F)CC1)c1conc1CF ; O=C(N[C@H](c1cn2nc(CC3C[C@@H](C(F)(F)F)CNC3=O)ccc2n1)C1CCC(F)(F)CC1)c1conc1CC(F)F] 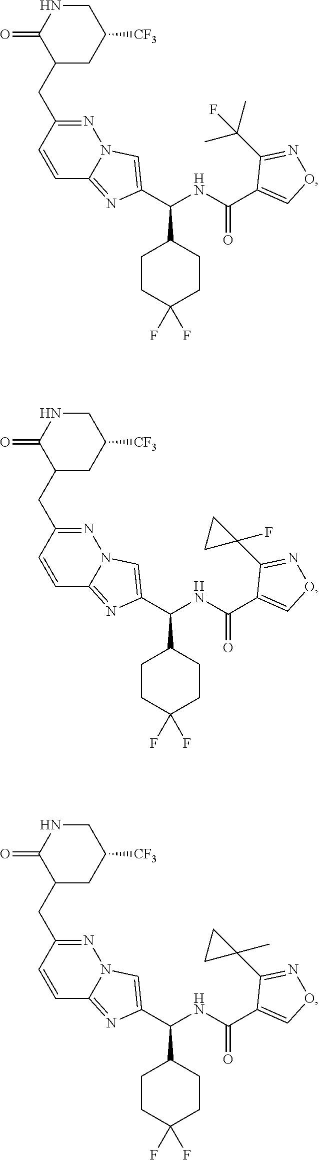 [SMILES: CC(C)(F)c1nocc1C(=O)N[C@H](c1cn2nc(CC3C[C@@H](C(F)(F)F)CNC3=O)ccc2n1)C1CCC(F)(F)CC1 ; O=C(N[C@H](c1cn2nc(CC3C[C@@H](C(F)(F)F)CNC3=O)ccc2n1)C1CCC(F)(F)CC1)c1conc1C1(F)CC1 ; CC1(c2nocc2C(=O)N[C@H](c2cn3nc(CC4C[C@@H](C(F)(F)F)CNC4=O)ccc3n2)C2CCC(F)(F)CC2)CC1] 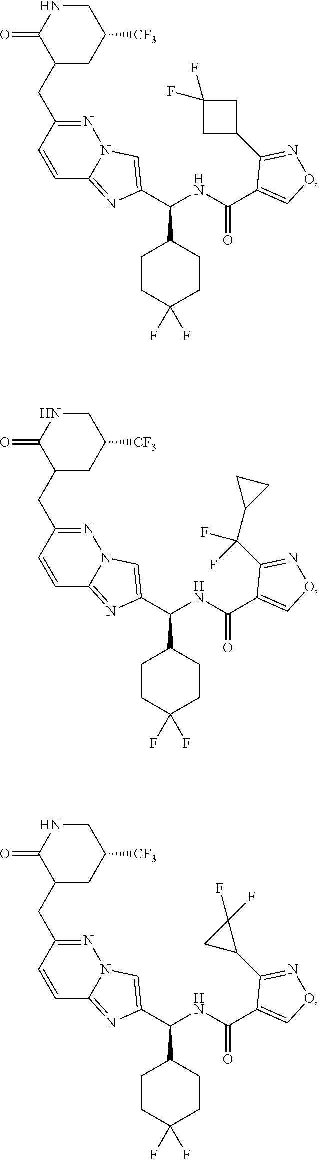 [SMILES: O=C(N[C@H](c1cn2nc(CC3C[C@@H](C(F)(F)F)CNC3=O)ccc2n1)C1CCC(F)(F)CC1)c1conc1C1CC(F)(F)C1 ; O=C(N[C@H](c1cn2nc(CC3C[C@@H](C(F)(F)F)CNC3=O)ccc2n1)C1CCC(F)(F)CC1)c1conc1C(F)(F)C1CC1 ; O=C(N[C@H](c1cn2nc(CC3C[C@@H](C(F)(F)F)CNC3=O)ccc2n1)C1CCC(F)(F)CC1)c1conc1C1CC1(F)F] 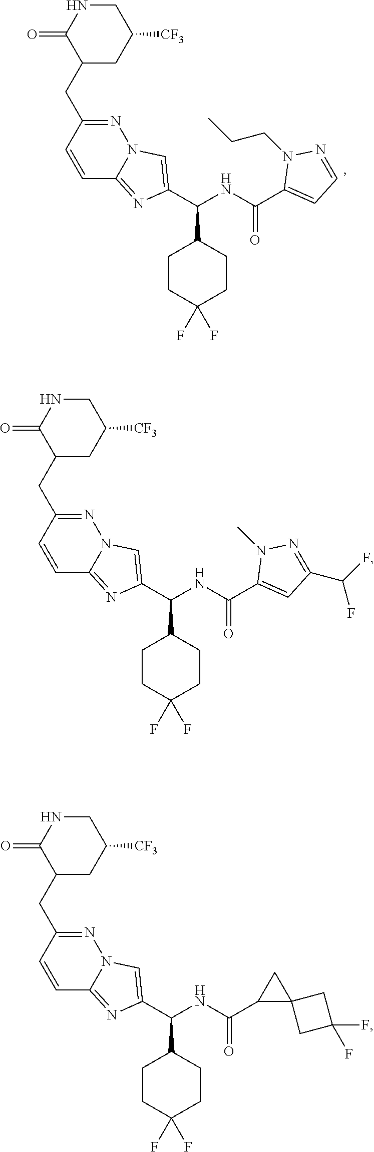 [SMILES: CCCn1nccc1C(=O)N[C@H](c1cn2nc(CC3C[C@@H](C(F)(F)F)CNC3=O)ccc2n1)C1CCC(F)(F)CC1 ; Cn1nc(C(F)F)cc1C(=O)N[C@H](c1cn2nc(CC3C[C@@H](C(F)(F)F)CNC3=O)ccc2n1)C1CCC(F)(F)CC1 ; O=C1NC[C@H](C(F)(F)F)CC1Cc1ccc2nc([C@@H](NC(=O)C3CC34CC(F)(F)C4)C3CCC(F)(F)CC3)cn2n1] 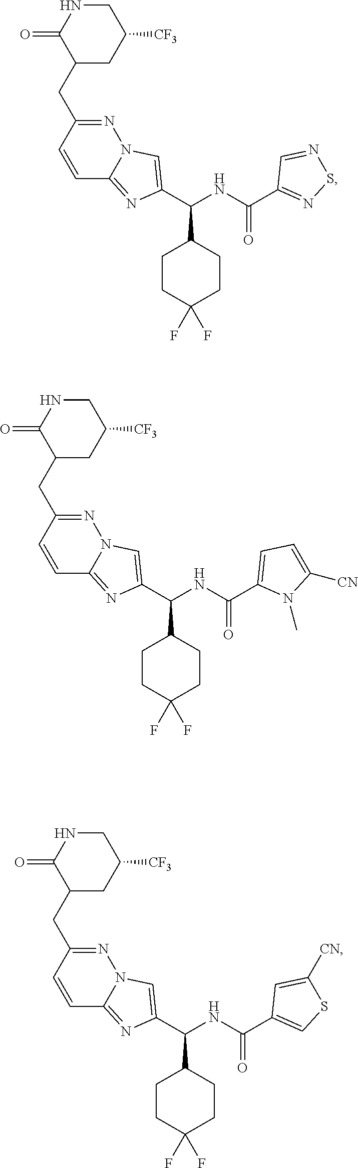 [SMILES: O=C(N[C@H](c1cn2nc(CC3C[C@@H](C(F)(F)F)CNC3=O)ccc2n1)C1CCC(F)(F)CC1)c1cnsn1 ; Cn1c(C#N)ccc1C(=O)N[C@H](c1cn2nc(CC3C[C@@H](C(F)(F)F)CNC3=O)ccc2n1)C1CCC(F)(F)CC1 ; N#Cc1cc(C(=O)N[C@H](c2cn3nc(CC4C[C@@H](C(F)(F)F)CNC4=O)ccc3n2)C2CCC(F)(F)CC2)cs1] 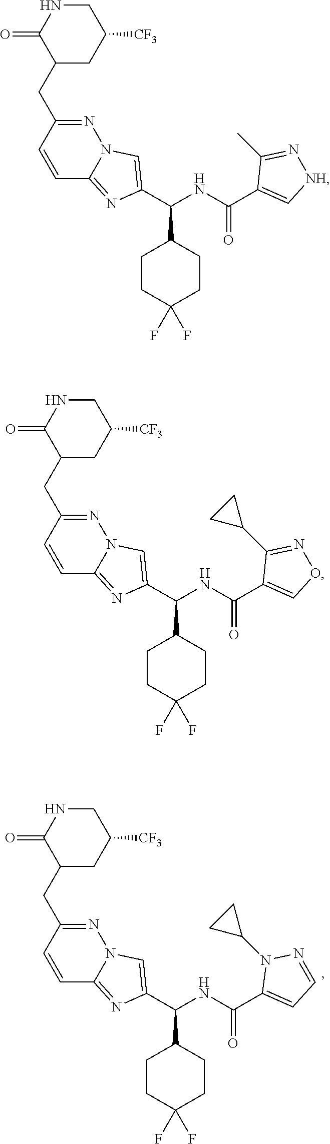 [SMILES: Cc1n[nH]cc1C(=O)N[C@H](c1cn2nc(CC3C[C@@H](C(F)(F)F)CNC3=O)ccc2n1)C1CCC(F)(F)CC1 ; O=C(N[C@H](c1cn2nc(CC3C[C@@H](C(F)(F)F)CNC3=O)ccc2n1)C1CCC(F)(F)CC1)c1conc1C1CC1 ; O=C(N[C@H](c1cn2nc(CC3C[C@@H](C(F)(F)F)CNC3=O)ccc2n1)C1CCC(F)(F)CC1)c1ccnn1C1CC1] 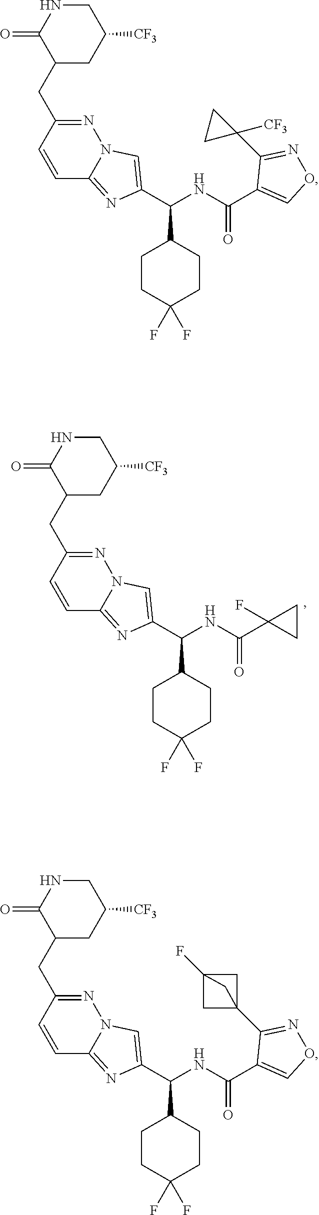 [SMILES: O=C(N[C@H](c1cn2nc(CC3C[C@@H](C(F)(F)F)CNC3=O)ccc2n1)C1CCC(F)(F)CC1)c1conc1C1(C(F)(F)F)CC1 ; O=C1NC[C@H](C(F)(F)F)CC1Cc1ccc2nc([C@@H](NC(=O)C3(F)CC3)C3CCC(F)(F)CC3)cn2n1 ; O=C(N[C@H](c1cn2nc(CC3C[C@@H](C(F)(F)F)CNC3=O)ccc2n1)C1CCC(F)(F)CC1)c1conc1C12CC(F)(C1)C2] 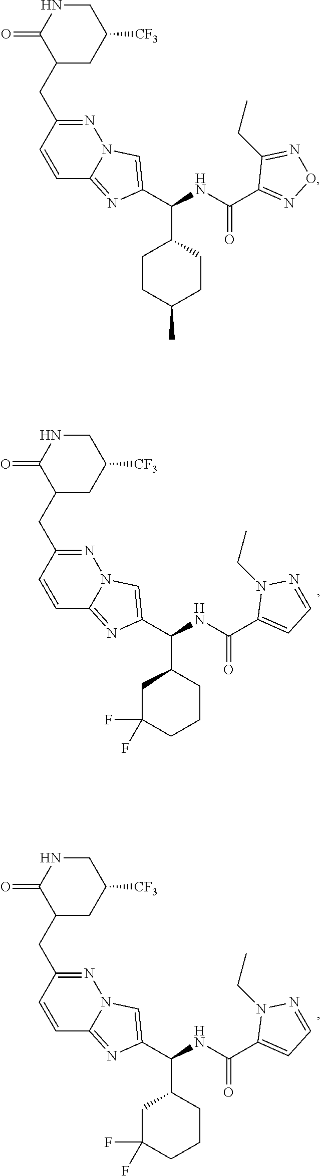 [SMILES: CCc1nonc1C(=O)N[C@H](c1cn2nc(CC3C[C@@H](C(F)(F)F)CNC3=O)ccc2n1)[C@H]1CC[C@H](C)CC1 ; CCn1nccc1C(=O)N[C@H](c1cn2nc(CC3C[C@@H](C(F)(F)F)CNC3=O)ccc2n1)[C@H]1CCCC(F)(F)C1 ; CCn1nccc1C(=O)N[C@H](c1cn2nc(CC3C[C@@H](C(F)(F)F)CNC3=O)ccc2n1)[C@@H]1CCCC(F)(F)C1] 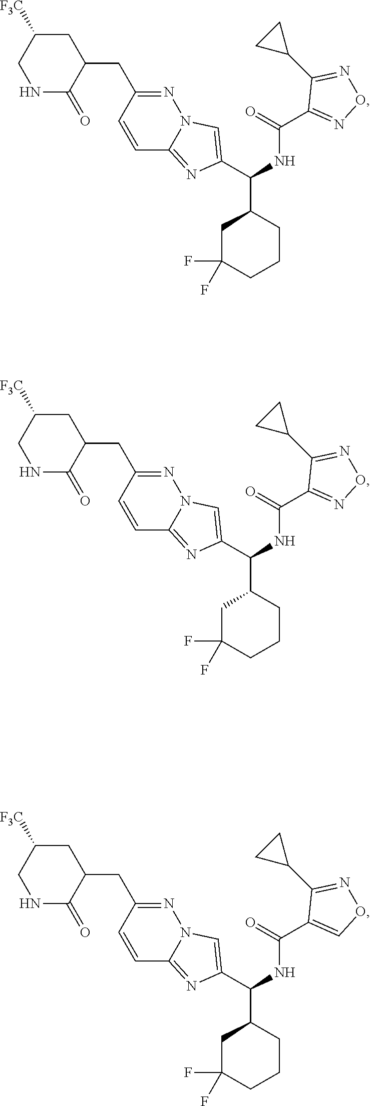 [SMILES: O=C(N[C@H](c1cn2nc(CC3C[C@@H](C(F)(F)F)CNC3=O)ccc2n1)[C@H]1CCCC(F)(F)C1)c1nonc1C1CC1 ; O=C(N[C@H](c1cn2nc(CC3C[C@@H](C(F)(F)F)CNC3=O)ccc2n1)[C@@H]1CCCC(F)(F)C1)c1nonc1C1CC1 ; O=C(N[C@H](c1cn2nc(CC3C[C@@H](C(F)(F)F)CNC3=O)ccc2n1)[C@H]1CCCC(F)(F)C1)c1conc1C1CC1] 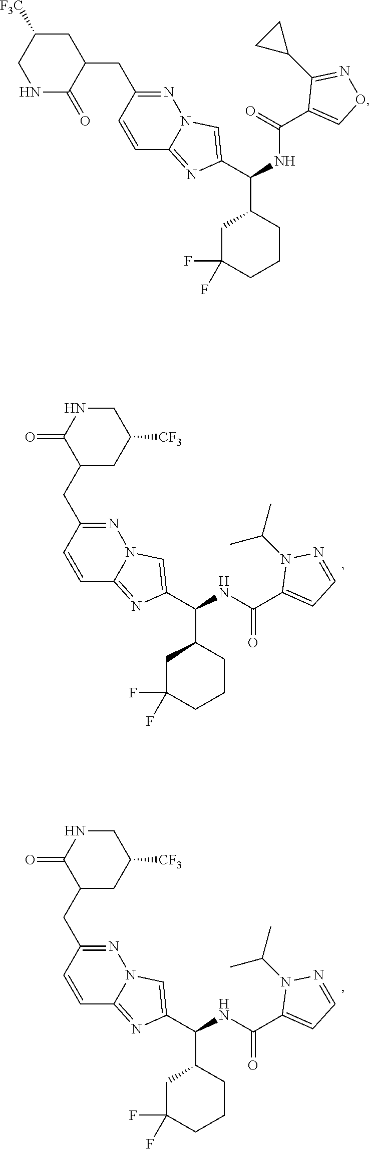 [SMILES: O=C(N[C@H](c1cn2nc(CC3C[C@@H](C(F)(F)F)CNC3=O)ccc2n1)[C@@H]1CCCC(F)(F)C1)c1conc1C1CC1 ; CC(C)n1nccc1C(=O)N[C@H](c1cn2nc(CC3C[C@@H](C(F)(F)F)CNC3=O)ccc2n1)[C@H]1CCCC(F)(F)C1 ; CC(C)n1nccc1C(=O)N[C@H](c1cn2nc(CC3C[C@@H](C(F)(F)F)CNC3=O)ccc2n1)[C@@H]1CCCC(F)(F)C1] 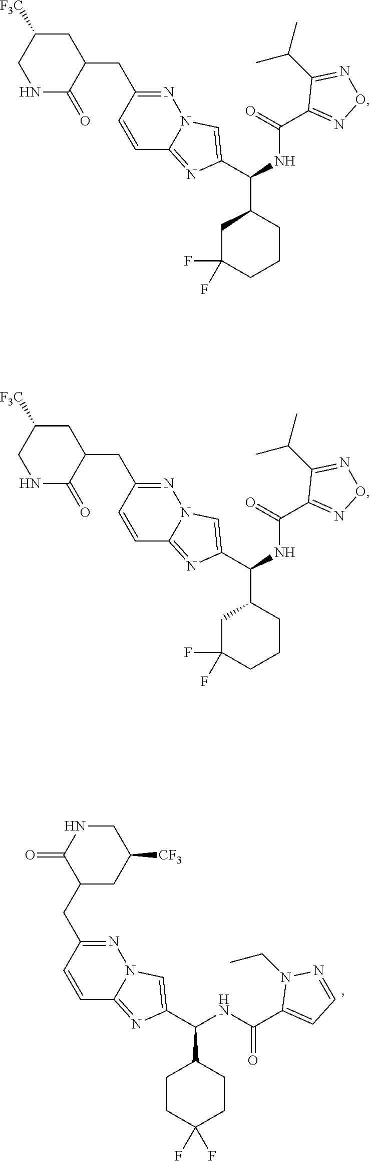 [SMILES: CC(C)c1nonc1C(=O)N[C@H](c1cn2nc(CC3C[C@@H](C(F)(F)F)CNC3=O)ccc2n1)[C@H]1CCCC(F)(F)C1 ; CC(C)c1nonc1C(=O)N[C@H](c1cn2nc(CC3C[C@@H](C(F)(F)F)CNC3=O)ccc2n1)[C@@H]1CCCC(F)(F)C1 ; CCn1nccc1C(=O)N[C@H](c1cn2nc(CC3C[C@H](C(F)(F)F)CNC3=O)ccc2n1)C1CCC(F)(F)CC1] 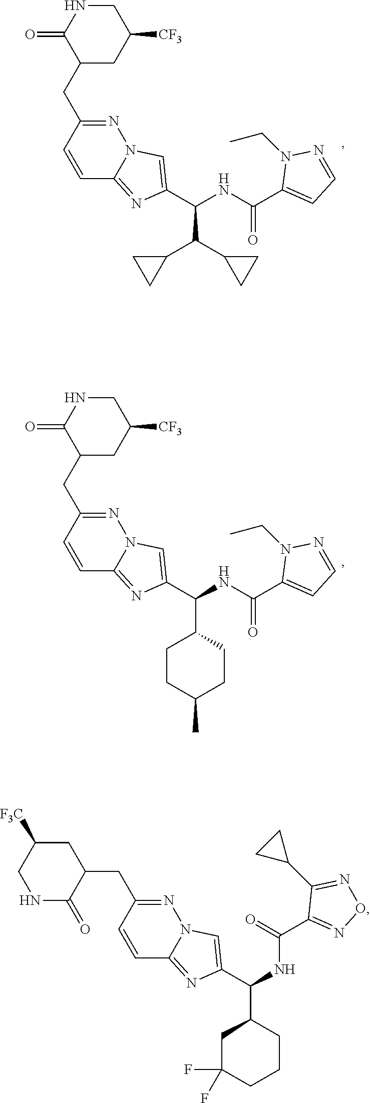 [SMILES: CCn1nccc1C(=O)N[C@H](c1cn2nc(CC3C[C@H](C(F)(F)F)CNC3=O)ccc2n1)C(C1CC1)C1CC1 ; CCn1nccc1C(=O)N[C@H](c1cn2nc(CC3C[C@H](C(F)(F)F)CNC3=O)ccc2n1)[C@H]1CC[C@H](C)CC1 ; O=C(N[C@H](c1cn2nc(CC3C[C@H](C(F)(F)F)CNC3=O)ccc2n1)[C@H]1CCCC(F)(F)C1)c1nonc1C1CC1] 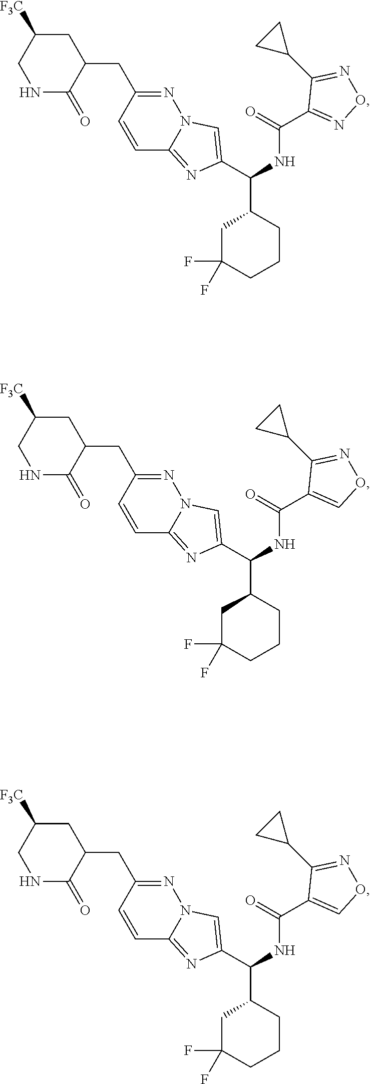 [SMILES: O=C(N[C@H](c1cn2nc(CC3C[C@H](C(F)(F)F)CNC3=O)ccc2n1)[C@@H]1CCCC(F)(F)C1)c1nonc1C1CC1 ; O=C(N[C@H](c1cn2nc(CC3C[C@H](C(F)(F)F)CNC3=O)ccc2n1)[C@H]1CCCC(F)(F)C1)c1conc1C1CC1 ; O=C(N[C@H](c1cn2nc(CC3C[C@H](C(F)(F)F)CNC3=O)ccc2n1)[C@@H]1CCCC(F)(F)C1)c1conc1C1CC1] 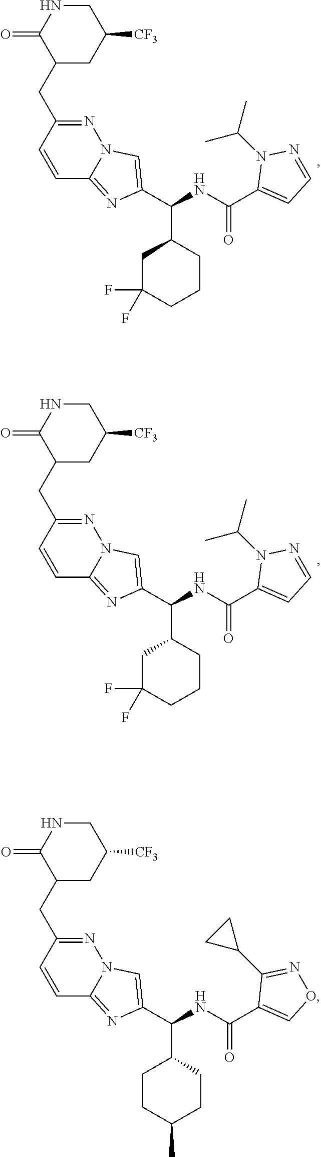 [SMILES: CC(C)n1nccc1C(=O)N[C@H](c1cn2nc(CC3C[C@H](C(F)(F)F)CNC3=O)ccc2n1)[C@H]1CCCC(F)(F)C1 ; CC(C)n1nccc1C(=O)N[C@H](c1cn2nc(CC3C[C@H](C(F)(F)F)CNC3=O)ccc2n1)[C@@H]1CCCC(F)(F)C1 ; C[C@H]1CC[C@H]([C@H](NC(=O)c2conc2C2CC2)c2cn3nc(CC4C[C@@H](C(F)(F)F)CNC4=O)ccc3n2)CC1] 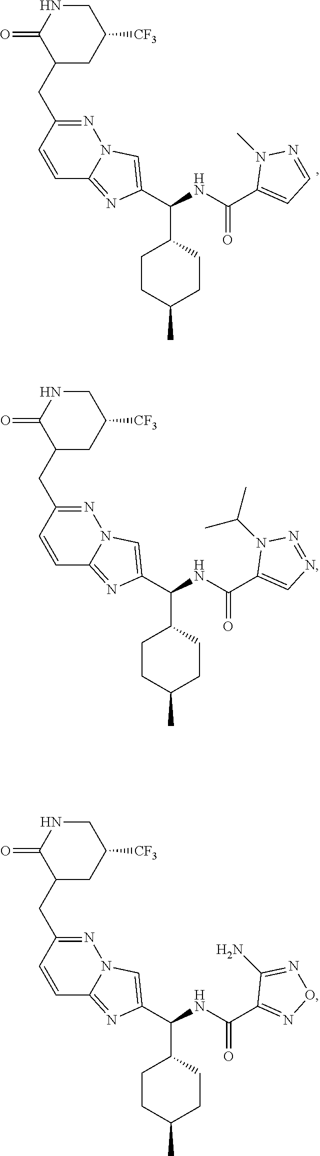 [SMILES: Cn1nccc1C(=O)N[C@H](c1cn2nc(CC3C[C@@H](C(F)(F)F)CNC3=O)ccc2n1)[C@H]1CC[C@H](C)CC1 ; CC(C)n1nncc1C(=O)N[C@H](c1cn2nc(CC3C[C@@H](C(F)(F)F)CNC3=O)ccc2n1)[C@H]1CC[C@H](C)CC1 ; C[C@H]1CC[C@H]([C@H](NC(=O)c2nonc2N)c2cn3nc(CC4C[C@@H](C(F)(F)F)CNC4=O)ccc3n2)CC1] 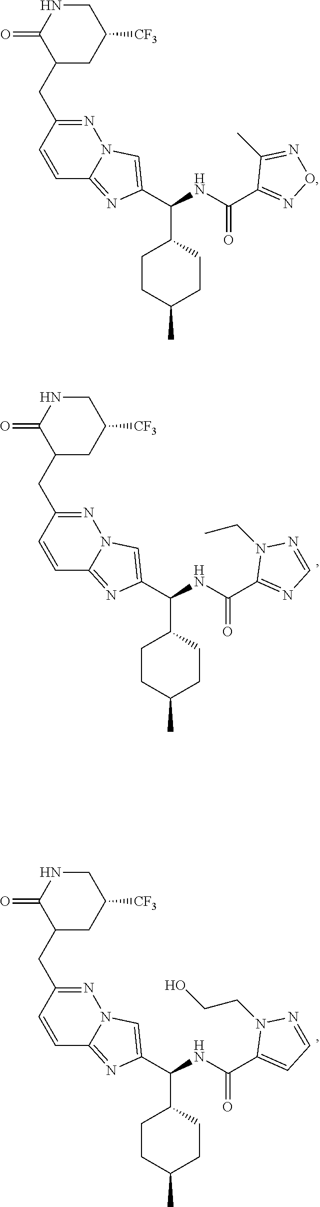 [SMILES: Cc1nonc1C(=O)N[C@H](c1cn2nc(CC3C[C@@H](C(F)(F)F)CNC3=O)ccc2n1)[C@H]1CC[C@H](C)CC1 ; CCn1ncnc1C(=O)N[C@H](c1cn2nc(CC3C[C@@H](C(F)(F)F)CNC3=O)ccc2n1)[C@H]1CC[C@H](C)CC1 ; C[C@H]1CC[C@H]([C@H](NC(=O)c2ccnn2CCO)c2cn3nc(CC4C[C@@H](C(F)(F)F)CNC4=O)ccc3n2)CC1] 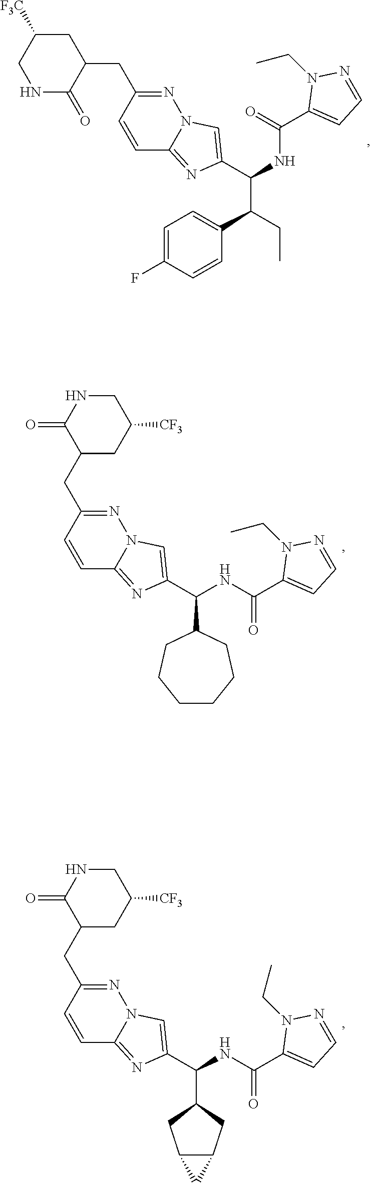 [SMILES: CC[C@@H](c1ccc(F)cc1)[C@H](NC(=O)c1ccnn1CC)c1cn2nc(CC3C[C@@H](C(F)(F)F)CNC3=O)ccc2n1 ; CCn1nccc1C(=O)N[C@H](c1cn2nc(CC3C[C@@H](C(F)(F)F)CNC3=O)ccc2n1)C1CCCCCC1 ; CCn1nccc1C(=O)N[C@H](c1cn2nc(CC3C[C@@H](C(F)(F)F)CNC3=O)ccc2n1)[C@@H]1C[C@H]2C[C@H]2C1] 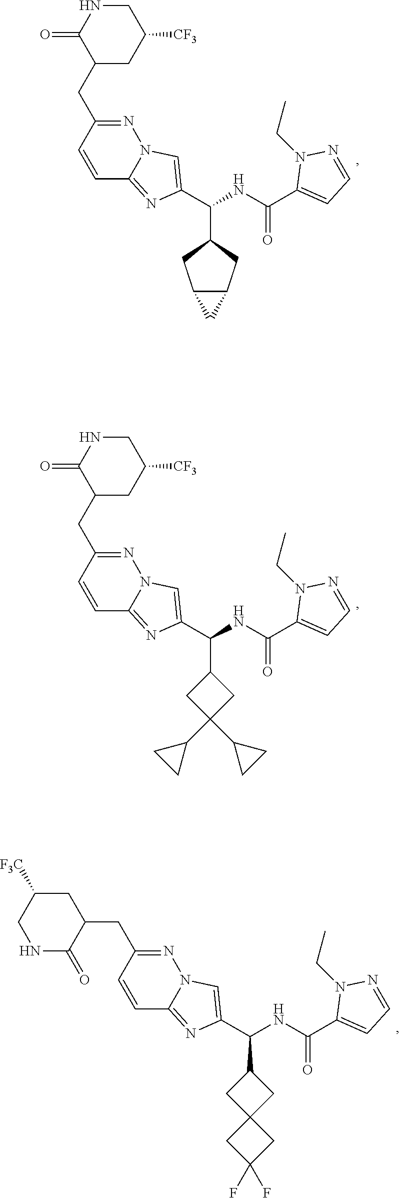 [SMILES: CCn1nccc1C(=O)N[C@@H](c1cn2nc(CC3C[C@@H](C(F)(F)F)CNC3=O)ccc2n1)[C@@H]1C[C@H]2C[C@H]2C1 ; CCn1nccc1C(=O)N[C@H](c1cn2nc(CC3C[C@@H](C(F)(F)F)CNC3=O)ccc2n1)C1CC(C2CC2)(C2CC2)C1 ; CCn1nccc1C(=O)N[C@H](c1cn2nc(CC3C[C@@H](C(F)(F)F)CNC3=O)ccc2n1)C1CC2(C1)CC(F)(F)C2] 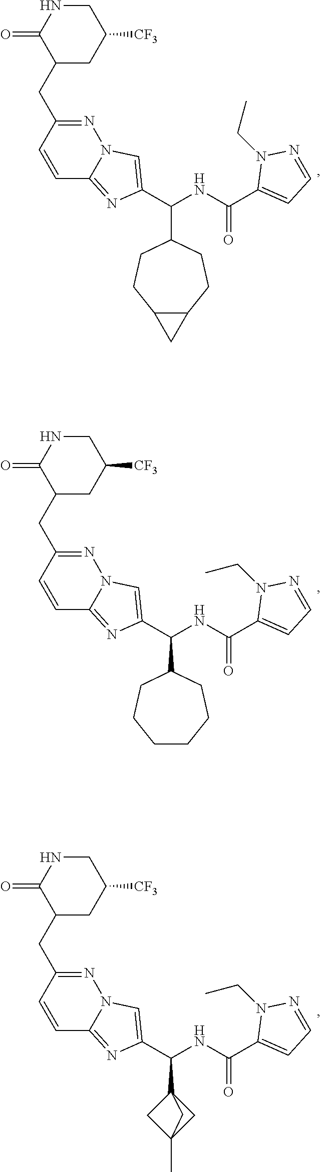 [SMILES: CCn1nccc1C(=O)NC(c1cn2nc(CC3C[C@@H](C(F)(F)F)CNC3=O)ccc2n1)C1CCC2CC2CC1 ; CCn1nccc1C(=O)N[C@H](c1cn2nc(CC3C[C@H](C(F)(F)F)CNC3=O)ccc2n1)C1CCCCCC1 ; CCn1nccc1C(=O)N[C@H](c1cn2nc(CC3C[C@@H](C(F)(F)F)CNC3=O)ccc2n1)C12CC(C)(C1)C2] 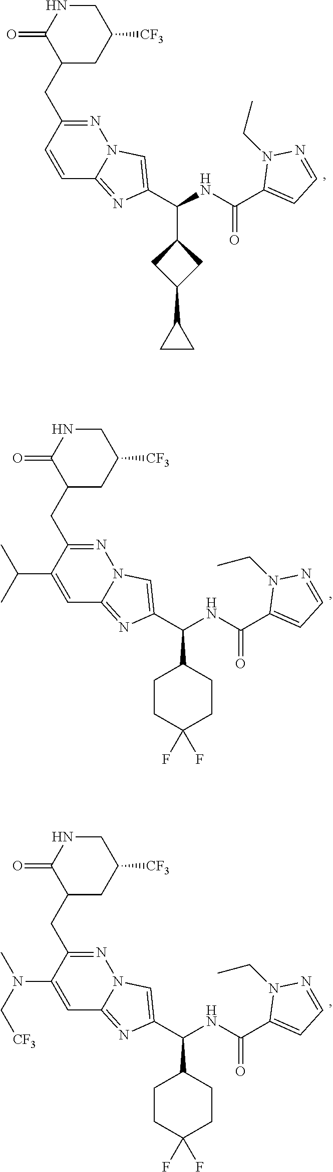 [SMILES: CCn1nccc1C(=O)N[C@H](c1cn2nc(CC3C[C@@H](C(F)(F)F)CNC3=O)ccc2n1)[C@H]1C[C@@H](C2CC2)C1 ; CCn1nccc1C(=O)N[C@H](c1cn2nc(CC3C[C@@H](C(F)(F)F)CNC3=O)c(C(C)C)cc2n1)C1CCC(F)(F)CC1 ; CCn1nccc1C(=O)N[C@H](c1cn2nc(CC3C[C@@H](C(F)(F)F)CNC3=O)c(N(C)CC(F)(F)F)cc2n1)C1CCC(F)(F)CC1] 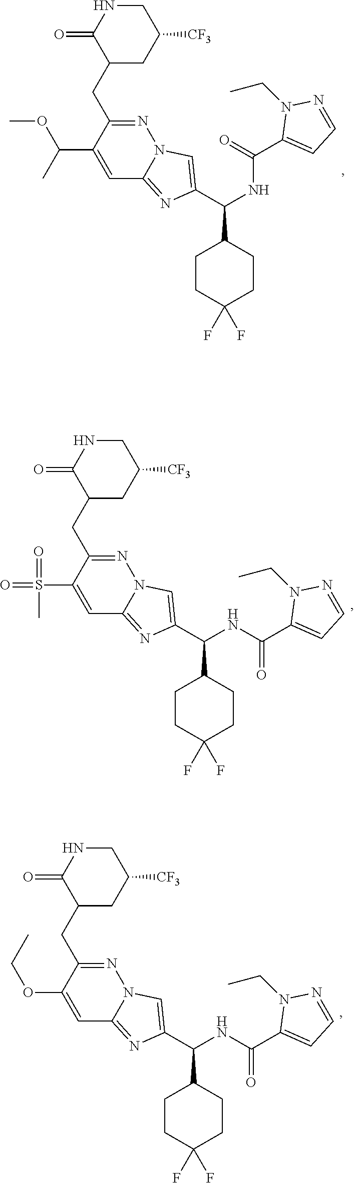 [SMILES: CCn1nccc1C(=O)N[C@H](c1cn2nc(CC3C[C@@H](C(F)(F)F)CNC3=O)c(C(C)OC)cc2n1)C1CCC(F)(F)CC1 ; CCn1nccc1C(=O)N[C@H](c1cn2nc(CC3C[C@@H](C(F)(F)F)CNC3=O)c(S(C)(=O)=O)cc2n1)C1CCC(F)(F)CC1 ; CCOc1cc2nc([C@@H](NC(=O)c3ccnn3CC)C3CCC(F)(F)CC3)cn2nc1CC1C[C@@H](C(F)(F)F)CNC1=O] 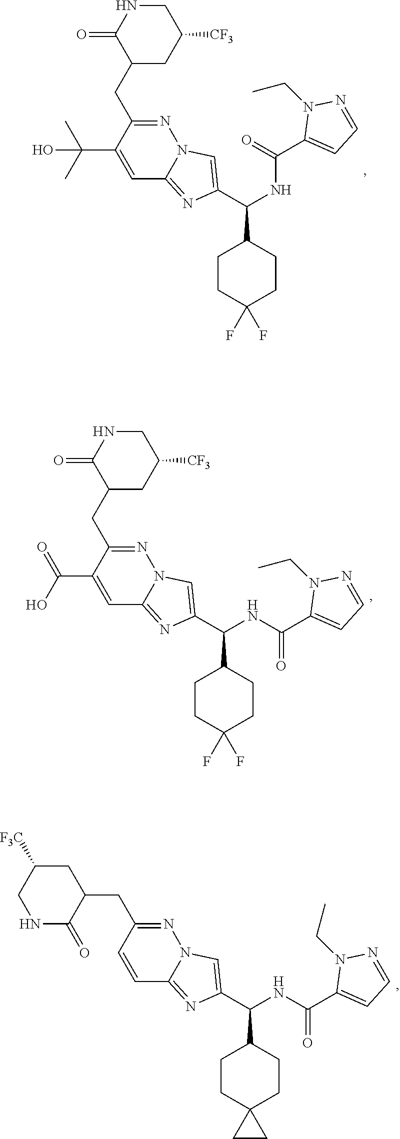 [SMILES: CCn1nccc1C(=O)N[C@H](c1cn2nc(CC3C[C@@H](C(F)(F)F)CNC3=O)c(C(C)(C)O)cc2n1)C1CCC(F)(F)CC1 ; CCn1nccc1C(=O)N[C@H](c1cn2nc(CC3C[C@@H](C(F)(F)F)CNC3=O)c(C(=O)O)cc2n1)C1CCC(F)(F)CC1 ; CCn1nccc1C(=O)N[C@H](c1cn2nc(CC3C[C@@H](C(F)(F)F)CNC3=O)ccc2n1)C1CCC2(CC1)CC2] 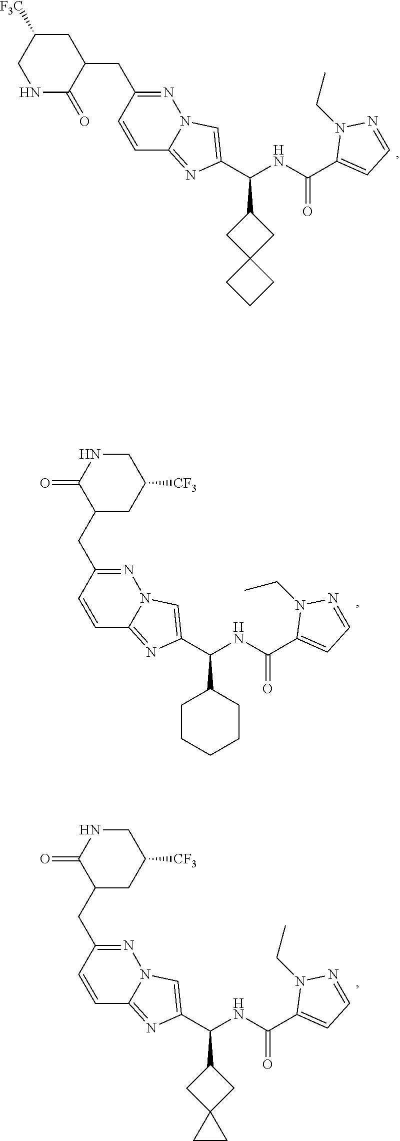 [SMILES: CCn1nccc1C(=O)N[C@H](c1cn2nc(CC3C[C@@H](C(F)(F)F)CNC3=O)ccc2n1)C1CC2(CCC2)C1 ; CCn1nccc1C(=O)N[C@H](c1cn2nc(CC3C[C@@H](C(F)(F)F)CNC3=O)ccc2n1)C1CCCCC1 ; CCn1nccc1C(=O)N[C@H](c1cn2nc(CC3C[C@@H](C(F)(F)F)CNC3=O)ccc2n1)C1CC2(CC2)C1] 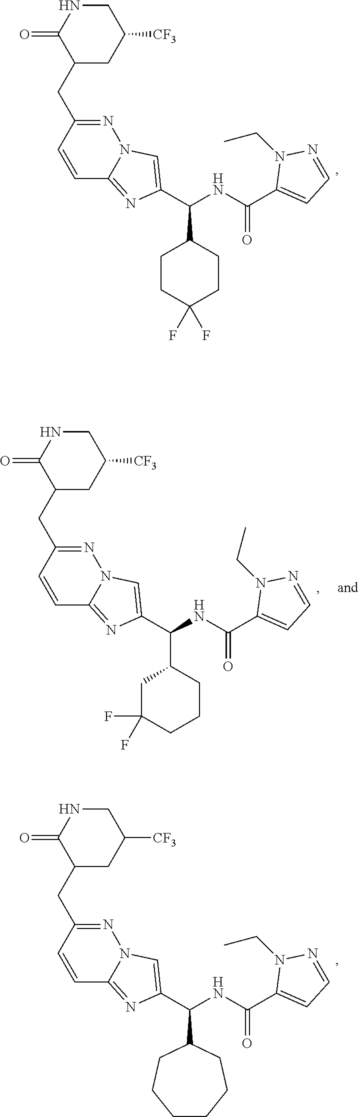 [SMILES: CCn1nccc1C(=O)N[C@H](c1cn2nc(CC3C[C@@H](C(F)(F)F)CNC3=O)ccc2n1)C1CCC(F)(F)CC1 ; CCn1nccc1C(=O)N[C@H](c1cn2nc(CC3C[C@@H](C(F)(F)F)CNC3=O)ccc2n1)[C@@H]1CCCC(F)(F)C1 ; CCn1nccc1C(=O)N[C@H](c1cn2nc(CC3CC(C(F)(F)F)CNC3=O)ccc2n1)C1CCCCCC1]
or pharmaceutically acceptable salts thereof.

71. A compound represented by structure of Formula (III): 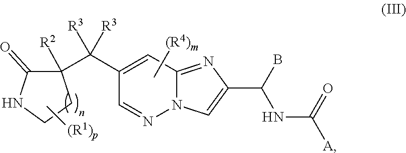
or a pharmaceutically acceptable salt thereof wherein:
A is selected from 5- to 6-membered heteroaryl and C₃₋₆ carbocycle, any of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹,
—N(R¹¹)S(O)₂R¹¹, —C(O)OR¹¹, —OC(O)R¹¹, —S(O)R¹¹, —S(O)₂R¹¹, —NO₂, —CN;
C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
C₃₋₁₀ carbocycle optionally substituted with one or more substituents independently selected from halogen, —OR¹¹A, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN;
B is selected from —C(H)(R⁵)₂ and C₃₋₁₀ carbocycle, wherein each C₃₋₁₀ carbocycle is optionally substituted with one or more substituents independently selected from:
halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
C₁₋₁₀ alkyl and C₃₋₁₀ carbocycle, any of which is optionally substituted with one or more substituents independently selected from halogen, —OR¹², —SR¹², —N(R¹²)₂,
—C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹²,
N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁵ is independently selected at each occurrence from (i), (ii), and (iii):
(i) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
(ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³,
—N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
(iii) C₃₋₁₀ carbocycle optionally substituted with one or more substituents independently selected from:
halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —S(O)R¹³, —S(O)₂R¹³, —NO₂, ═O, ═S, ═N(R¹³), and —CN;
each R¹ is independently selected at each occurrence from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN; and C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN;
R² is selected from hydrogen, halogen, —OR¹⁵, —SR¹¹, —N(R¹¹)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN; or
each R⁴ is independently selected at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, and —CN;
R¹¹, R^{11A}, R¹², R¹³, R¹⁴, R¹⁵, R¹⁶, and R¹⁷ are each independently selected at each occurrence from:
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2;
m is selected from 0, 1 and 2; and
p is selected from 0, 1, 2, 3, 4, 5, and 6.

72. The compound or salt of claim 71, wherein A is selected from 5- to 6-membered heteroaryl and C₃₋₆ carbocycle, any of which is optionally substituted with one or more substituents independently selected from C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂—C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

73. The compound or salt of claim 72, wherein A is 5- to 6-membered heteroaryl optionally substituted with C₃₋₁₀ carbocycle optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), —CN; and C₁₋₁₀ alkyl optionally substituted with one or more substituents selected from: halogen, —OR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —NO₂, ═O, ═N(R^{11A}), and —CN.

74. The compound or salt of claim 73, wherein A is 5-membered heteroaryl optionally substituted with C₃₋₁₀ carbocycle optionally substituted with one or more substituents independently selected from halogen, —OR^{11A}, —N(R^{11A})₂, —C(O)R^{11A}, —NO₂, —CN; and C₁₋₁₀ alkyl optionally substituted with one or more substituents selected from: halogen, —OR^{11A}, —N(R^{11A})₂, —C(O)R^{11A}, —NO₂, and —CN.

75. The compound or salt of claim 74, wherein A is selected from pyrazolyl, imidazolyl, oxazolyl, isoxazolyl, oxadiazolyl, each of which optionally substituted with C₃₋₆ cycloalkyl optionally substituted with one or more substituents independently selected from halogen, —OR^{11A}, —N(R^{11A})₂, —C(O)R^{11A}, —NO₂, —CN, C₁₋₆ alkyl, and C₁₋₆ haloalkyl.

76. The compound or salt of claim 75, wherein A is selected from oxazolyl, isoxazolyl, oxadiazolyl, each of which is optionally substituted with C₃₋₆ cycloalkyl optionally substituted with one or more substituents independently selected from halogen, —OR^{11A}, —N(R^{11A})₂, —C(O)R^{11A}, —NO₂, —CN, C₁₋₆ alkyl, and C₁₋₆ haloalkyl.

77. The compound or salt of claim 76, wherein A is selected from:

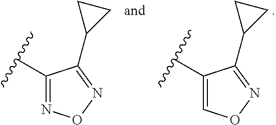 [SMILES: *c1nonc1C1CC1 ; "and" ; *c1conc1C1CC1 ; "."]

78. The compound or salt of claim 71, wherein Formula III is represented by structure of Formula (IV): 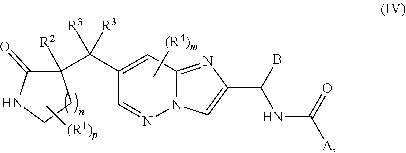
or a pharmaceutically acceptable salt thereof wherein:
A is selected from 5- to 6-membered heteroaryl and C₃₋₆ carbocycle, any of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹,
—N(R¹¹)S(O)₂R¹¹, —C(O)OR¹¹, —OC(O)R¹¹, —S(O)R¹¹, —S(O)₂R¹¹, —NO₂, —CN; and
C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂,
—N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN;
B is selected from —C(H)(R⁵)₂ and C₃₋₁₀ carbocycle, wherein each C₃₋₁₀ carbocycle is optionally substituted with one or more substituents independently selected from:
halogen, —OR¹², —SR¹², —N(R¹²)₂, —C(O)R¹², —C(O)N(R¹²)₂, —N(R¹²)C(O)R¹²,
—N(R¹²)S(O)₂R¹², —C(O)OR¹², —OC(O)R¹², —S(O)R¹², —S(O)₂R¹², —NO₂, ═O, ═S, ═N(R¹²), —CN;
C₁₋₁₀ alkyl and C₃₋₁₀ carbocycle, any of which is optionally substituted with one or more substituents independently selected from halogen, —OR¹², —SR¹², —N(R¹²)₂,
—C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹²,
—N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁵ is independently selected at each occurrence from (i), (ii), and (iii):
(i) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
(ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³,
—N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
(iii) C₃₋₁₀ carbocycle optionally substituted with one or more substituents independently selected from:
halogen, —OR¹³, —SR¹³, —N(R¹³)₂, —C(O)R¹³, —C(O)N(R¹³)₂, —N(R¹³)C(O)R¹³,
—N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
C₁₋₁₀ alkyl optionally substituted with one or more substituents independently selected from halogen, —OR¹³, —SR¹³, —N(R¹³)₂, —C(O)R¹³, —C(O)N(R¹³)₂,
—N(R¹³)C(O)R¹³, —C(O)OR¹³, —OC(O)R¹³, —S(O)R¹³, —S(O)₂R¹³, —NO₂, ═O, ═S, ═N(R¹³), and —CN;
each R¹ is independently selected at each occurrence from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴,
—S(O)₂R¹⁴, —NO₂, ═O, ═S, ═N(R¹⁴), —CN; and C₁₋₁₀ alkyl optionally substituted with one or more substituents independently selected from halogen, —OR¹⁴, —SR¹⁴, —N(R¹⁴)₂, —C(O)R¹⁴,
—C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN;
R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵,
—OC(O)R¹⁵, —S(O)R¹⁵, —S(O)₂R¹⁵, —NO₂, ═O, ═S, ═N(R¹⁵), and —CN;
each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂,
—N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN; or
each R⁴ is independently selected at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷,
—S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂,
—N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN;
R¹¹, R¹², R¹³, R¹⁴, R¹⁵, R¹⁶, and R¹⁷ are each independently selected at each occurrence from:
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2;
m is selected from 0, 1 and 2; and
p is selected from 0, 1, 2, 3, 4, 5, and 6.

79. The compound or salt of claim 78, wherein n is 1.

80. The compound or salt of claim 78, wherein Formula (IV) is represented by the structure of Formula (IV-a):

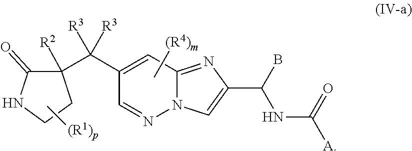

81. The compound or salt of claim 78, wherein n is 2.

82. The compound or salt of claim 78, wherein Formula (IV) is represented by the structure of Formula (IV-b):

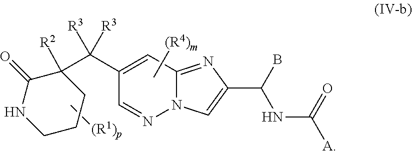

83. The compound or salt of claim 78, wherein A is 5-membered heteroaryl optionally substituted with one or more substituents independently selected from:
halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN; and
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

84. The compound or salt of claim 83, wherein A is selected from pyrazolyl, imidazolyl, oxazolyl, isoxazolyl, oxadiazolyl, triazolyl, and tetrazolyl, any one of which is optionally substituted with one or more C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

85. The compound or salt of claim 84, wherein A is pyrazolyl optionally substituted with C₁₋₆ alkyl optionally substituted with one or more substituents independently selected from halogen, —OR¹¹, —N(R¹¹)₂, —C(O)R¹¹, —NO₂, and —CN.

86. The compound or salt of claim 85, wherein A is pyrazolyl optionally substituted with methyl, ethyl, isopropyl, and propyl.

87. The compound or salt of claim 86, wherein A is

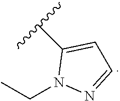 [SMILES: *c1ccnn1CC ; "."]

88. The compound or salt of claim 83 wherein A is selected from pyrazolyl, oxazolyl, isoxazolyl, and oxadiazolyl, any of which is optionally substituted with one or more C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

89. The compound or salt of claim 88, wherein A is selected from pyrazolyl, oxazolyl, and isoxazolyl, any of which is optionally substituted with one or more C₁₋₆ alkyl and C₁₋₆ haloalkyl.

90. The compound or salt of claim 89, wherein A is selected from

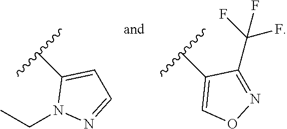

91. The compound or salt of claim 78, wherein B is —C(H)(R⁵)₂.

92. The compound or salt of claim 91, wherein each R⁵ of —C(H)(R⁵)₂ is independently selected from:
halogen, —OR¹³, —N(R¹³)₂, —C(O)R¹³, —C(O)OR¹³, —NO₂, —CN;
C₁₋₆ alkyl optionally substituted with one or more substituents independently selected from halogen, —OR¹³, —N(R¹³)₂, —C(O)R¹³, —C(O)OR¹³, —NO₂, ═O, ═S, ═N(R¹³), and —CN; and
C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl.

93. The compound or salt of claim 92, wherein each R⁵ of —C(H)(R⁵)₂ is independently selected from C₃₋₆ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl.

94. The compound or salt of claim 93, wherein —CH(R⁵)₂ is

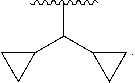 [SMILES: *C(C1CC1)C1CC1]

95. The compound or salt of claim 92, wherein each R⁵ of —C(H)(R⁵)₂ is independently selected from:
C₁₋₆ alkyl optionally substituted with one or more substituents independently selected from halogen, —OR¹³, —N(R¹³)₂, —C(O)R¹³, —C(O)OR¹³, —NO₂, ═O, ═S, ═N(R¹³), and —CN; and
C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl.

96. The compound or salt of claim 95, wherein each R⁵ of —C(H)(R⁵)₂ is independently selected from C₁₋₆ alkyl, C₁₋₆ haloalkyl, C₃₋₇ cycloalkyl, and phenyl, wherein the C₃₋₇ cycloalkyl and phenyl are each optionally substituted with one or more substituents independently selected from halogen, C₁₋₆ alkyl, and C₁₋₆ haloalkyl.

97. The compound or salt of claim 96, wherein —CH(R⁵)₂ is selected from

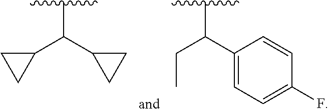 [SMILES: *C(C1CC1)C1CC1 ; "and" ; *C(CC)c1ccc(F)cc1]

98. The compound or salt of claim 78, wherein B is C₆₋₁₀ carbocycle optionally substituted with one or more substituents independently selected from: halogen, —OR¹², N(R¹²)₂, —C(O)R¹², —NO₂, ═O, ═S, ═N(R¹²), —CN, and C₁₋₆ alkyl optionally substituted with one or more substituents independently selected from halogen, —OR¹², N(R¹²)₂, —C(O)R¹², —NO₂, ═O, ═S, ═N(R¹²), and —CN.

99. The compound or salt of claim 98, wherein B is selected from cyclohexyl and cycloheptyl, either of which is optionally substituted with one or more substituents independently selected from: halogen, —OR¹², N(R¹²)₂, —NO₂, —CN, and C₁₋₆ alkyl optionally substituted with one or more substituents independently selected from halogen, —OR¹², N(R¹²)₂, —C(O)R¹², —NO₂, ═O, ═S, ═N(R¹²), and —CN.

100. The compound or salt of claim 99, wherein B is

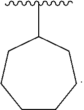 [SMILES: *C1CCCCCC1]

101. The compound or salt of claim 99, wherein B is selected from

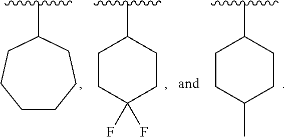

102. The compound or salt of claim 78, wherein m is 0.

103. The compound or salt of claim 78, wherein each R³ is independently selected at each occurrence from hydrogen, halogen, —OR¹⁶, —N(R¹⁶)₂, —NO₂, —CN, C₁₋₃ alkyl, and C₁₋₃ haloalkyl.

104. The compound or salt of claim 103, wherein each R³ is hydrogen.

105. The compound or salt of claim 78, wherein R² is hydrogen.

106. The compound or salt of claim 78, wherein p is selected from 0, 1, 2, 3, and 4.

107. The compound or salt of claim 78, wherein each R¹ is independently selected at each occurrence from halogen, —OR¹⁴, —N(R¹⁴)₂, —NO₂, —CN, C₁₋₆ alkyl, and C₁₋₆ haloalkyl.

108. The compound or salt of claim 107 wherein each R¹ is independently selected at each occurrence from halogen and —CF₃.

109. The compound or salt of claim 78, wherein Formula (III) is selected from: 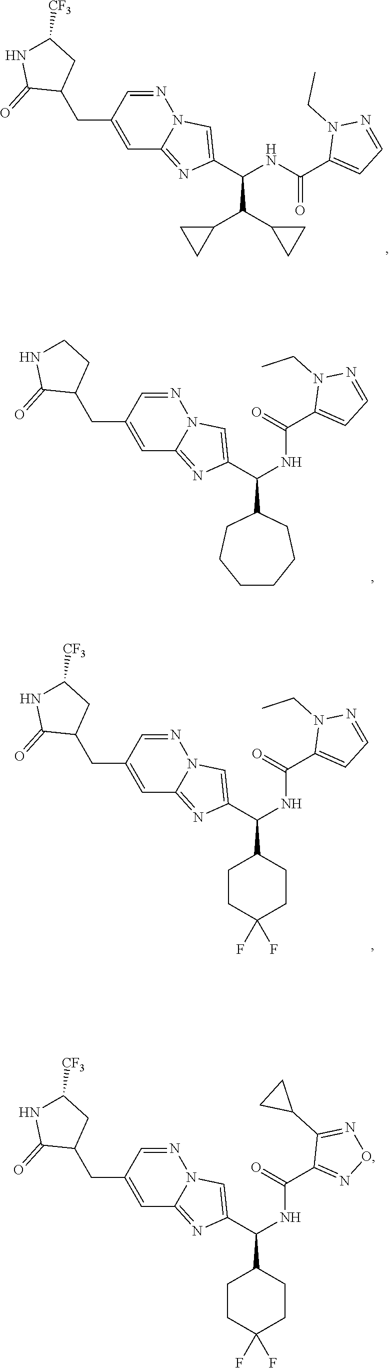 [SMILES: CCn1nccc1C(=O)N[C@H](c1cn2ncc(CC3C[C@@H](C(F)(F)F)NC3=O)cc2n1)C(C1CC1)C1CC1 ; CCn1nccc1C(=O)N[C@H](c1cn2ncc(CC3CCNC3=O)cc2n1)C1CCCCCC1 ; CCn1nccc1C(=O)N[C@H](c1cn2ncc(CC3C[C@@H](C(F)(F)F)NC3=O)cc2n1)C1CCC(F)(F)CC1 ; O=C(N[C@H](c1cn2ncc(CC3C[C@@H](C(F)(F)F)NC3=O)cc2n1)C1CCC(F)(F)CC1)c1nonc1C1CC1] 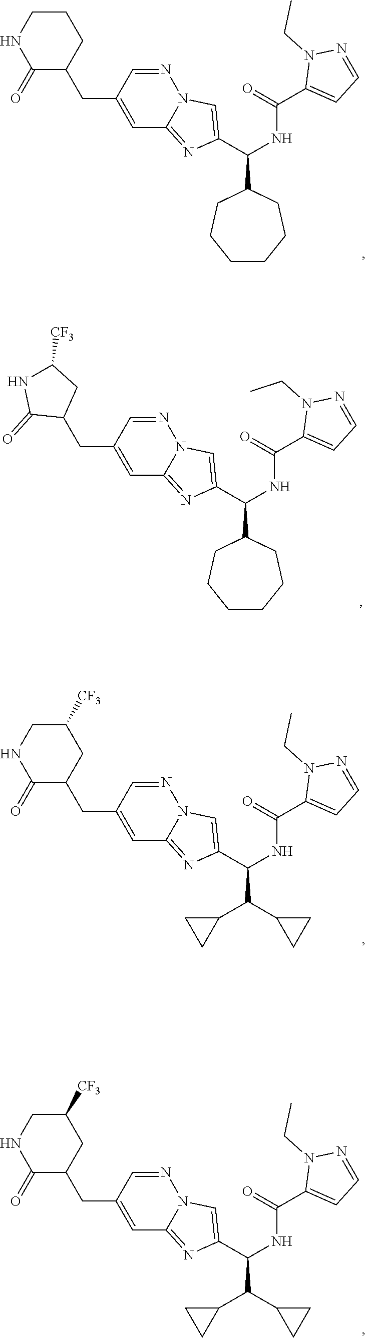 [SMILES: CCn1nccc1C(=O)N[C@H](c1cn2ncc(CC3CCCNC3=O)cc2n1)C1CCCCCC1 ; CCn1nccc1C(=O)N[C@H](c1cn2ncc(CC3C[C@@H](C(F)(F)F)NC3=O)cc2n1)C1CCCCCC1 ; CCn1nccc1C(=O)N[C@H](c1cn2ncc(CC3C[C@@H](C(F)(F)F)CNC3=O)cc2n1)C(C1CC1)C1CC1 ; CCn1nccc1C(=O)N[C@H](c1cn2ncc(CC3C[C@H](C(F)(F)F)CNC3=O)cc2n1)C(C1CC1)C1CC1] 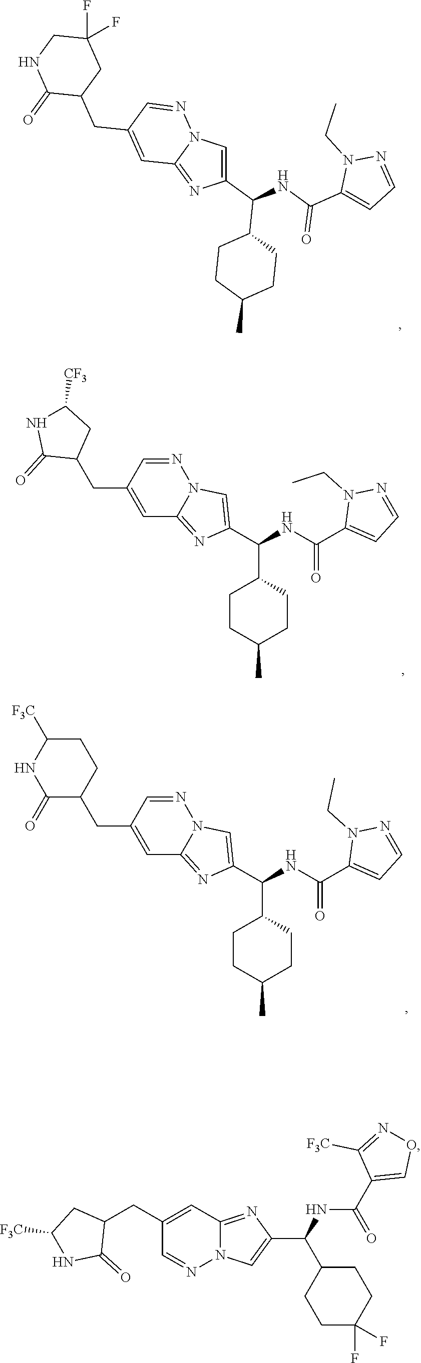 [SMILES: CCn1nccc1C(=O)N[C@H](c1cn2ncc(CC3CC(F)(F)CNC3=O)cc2n1)[C@H]1CC[C@H](C)CC1 ; CCn1nccc1C(=O)N[C@H](c1cn2ncc(CC3C[C@@H](C(F)(F)F)NC3=O)cc2n1)[C@H]1CC[C@H](C)CC1 ; CCn1nccc1C(=O)N[C@H](c1cn2ncc(CC3CCC(C(F)(F)F)NC3=O)cc2n1)[C@H]1CC[C@H](C)CC1 ; O=C(N[C@H](c1cn2ncc(CC3C[C@@H](C(F)(F)F)NC3=O)cc2n1)C1CCC(F)(F)CC1)c1conc1C(F)(F)F] 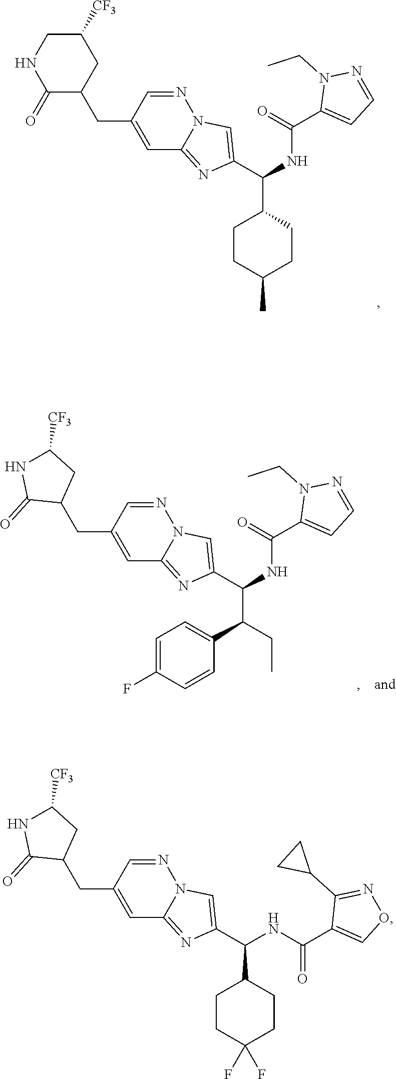 [SMILES: CCn1nccc1C(=O)N[C@H](c1cn2ncc(CC3C[C@@H](C(F)(F)F)CNC3=O)cc2n1)[C@H]1CC[C@H](C)CC1 ; CC[C@@H](c1ccc(F)cc1)[C@H](NC(=O)c1ccnn1CC)c1cn2ncc(CC3C[C@@H](C(F)(F)F)NC3=O)cc2n1 ; O=C(N[C@H](c1cn2ncc(CC3C[C@@H](C(F)(F)F)NC3=O)cc2n1)C1CCC(F)(F)CC1)c1conc1C1CC1]
or pharmaceutically acceptable salts thereof.

110. A pharmaceutical composition comprising a compound or salt of claim 1 and a pharmaceutically acceptable excipient.

111. A method of modulating IL-17A in a subject in need thereof, comprising administering to the subject a compound or salt of claim 1.

112. A method of treating an inflammatory disease or condition comprising administering to a subject in need thereof a compound or salt of claim 1.

113. The method of claim 112 wherein the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, palmoplantar psoriasis, spondyloarthritis, and Non-infectious Uveitis.
